(19) United States
(12) Patent Application Publication
(10) Pub. No.: US 2003/0194702 A1
(43) Pub. Date: Oct. 16, 2003

(54) Novel compositions and methods for cancer

(76) Inventors: Eric K. Engelhard, Davis, CA (US); David W. Morris, Davis, CA (US)

Correspondence Address: Gladys H. Monroy, Morrison & Foerster LLP, 755 Page Mill Road, Palo Alto, CA, 94304-1018, US

(21) Appl. No.: 10/004,113

(22) Filed: Oct. 23, 2001

Related U.S. Application Data
(63) Continuation-in-part of application No. 09/747,377, filed on Dec. 22, 2000.
(63) Continuation-in-part of application No. 09/798,586, filed on Mar. 2, 2001.

(51) Int. Cl. C12Q 1/68; G01N 33/574; C12P 21/02; C12N 5/06; C07H 21/04; C12N 9/64

(52) U.S. Cl. 435/6; 435/7.23; 435/69.3; 435/226; 435/320.1; 435/325; 536/23.2

(57) ABSTRACT

The present invention relates to novel sequences for use in diagnosis and treatment of carcinomas, especially lymphoma carcinomas. In addition, the present invention describes the use of novel compositions for use in screening methods.

The present application is a continuing application of U.S. Ser. Nos. 09/747,377, filed Dec. 22, 2000 and 09/798,586. filed Mar. 2, 2001, both of which are expressly incorporated herein by reference.

### FIELD OF THE INVENTION

The present invention relates to novel sequences for use in diagnosis and treatment of cancer, especially carcinomas, as well as the use of the novel compositions in screening methods.

### BACKGROUND OF THE INVENTION

Oncogenes are genes that can cause cancer. Carcinogenesis can occur by a wide variety of mechanisms, including infection of cells by viruses containing oncogenes, activation of protooncogenes in the host genome, and mutations of protooncogenes and tumor suppressor genes.

There are a number of viruses known to be involved in human cancer as well as in animal cancer. Of particular interest here are viruses that do not contain oncogenes themselves; these are slow-transforming retroviruses. They induce tumors by integrating into the host genome and affecting neighboring protooncogenes in a variety of ways, including promoter insertion, enhancer insertion, and/or truncation of a protooncogene or tumor suppressor gene. The analysis of sequences at or near the insertion sites led to the identification of a number of new protooncogenes.

With respect to lymphoma and leukemia, murine leukemia retrovirus (MuLV), such as SL3-3 or Akv, is a potent inducer of tumors when inoculated into susceptible newborn mice, or when carried in the germline. A number of sequences have been identified as relevant in the induction of lymphoma and leukemia by analyzing the insertion sites; see Sorensen et al., J. of Virology 74:2161 (2000); Hansen et al., Genome Res. 10(2):237-43 (2000); Sorensen et al., J. Virology 70:4063 (1996); Sorensen et al., J. Virology 67:7118 (1993); Joosten et al., Virology 268:308 (2000); and Li et al., Nature Genetics 23:348 (1999); all of which are expressly incorporated by reference herein.

Accordingly, it is an object of the invention to provide sequences involved in cancer and in particular in oncogenesis.

### SUMMARY OF THE INVENTION

In accordance with the objects outlined above, the present invention provides methods for screening for compositions which modulate carcinomas, especially lymphoma and leukemia. Also provided herein are methods of inhibiting proliferation of a cell, preferably a lymphoma cell. Methods of treatment of carcinomas, including diagnosis, are also provided herein.

In one aspect, a method of screening drug candidates comprises providing a cell that expresses a carcinoma associated (CA) gene or fragments thereof. Preferred embodiments of CA genes are genes which are differentially expressed in cancer cells, preferably lymphatic, breast, prostate or epithelial cells, compared to other cells. Preferred embodiments of CA genes used in the methods herein include, but are not limited to the nucleic acids selected from Tables 1-10. The method further includes adding a drug candidate to the cell and determining the effect of the drug candidate on the expression of the CA gene.

In one embodiment, the method of screening drug candidates includes comparing the level of expression in the absence of the drug candidate to the level of expression in the presence of the drug candidate.

Also provided herein is a method of screening for a bioactive agent capable of binding to a CA protein (CAP), the method comprising combining the CAP and a candidate bioactive agent, and determining the binding of the candidate agent to the CAP.

Further provided herein is a method for screening for a bioactive agent capable of modulating the activity of a CAP. In one embodiment, the method comprises combining the CAP and a candidate bioactive agent, and determining the effect of the candidate agent on the bioactivity of the CAP.

Also provided is a method of evaluating the effect of a candidate carcinoma drug comprising administering the drug to a patient and removing a cell sample from the patient. The expression profile of the cell is then determined. This method may further comprise comparing the expression profile of the patient to an expression profile of a heathy individual.

In a further aspect, a method for inhibiting the activity of an CA protein is provided. In one embodiment, the method comprises administering to a patient an inhibitor of a CA protein preferably selected from the group consisting of the sequences outlined in Tables 1-10 or their complements.

A method of neutralizing the effect of a CA protein, preferably a protein encoded by a nucleic acid selected from the group of sequences outlined in Tables 1-10, is also provided. Preferably, the method comprises contacting an agent specific for said protein with said protein in an amount sufficient to effect neutralization.

Moreover, provided herein is a biochip comprising a nucleic acid segment which encodes a CA protein, preferably selected from the sequences outlined in Tables 1-10.

Also provided herein is a method for diagnosing or determining the propensity to carcinomas, especially lymphoma or leukemia by sequencing at least one carcinoma or lymphoma gene of an individual. In yet another aspect of the invention, a method is provided for determining carcinoma including lymphoma and leukemia gene copy number in an individual.

Novel sequences are also provided herein. Other aspects of the invention will become apparent to the skilled artisan by the following description of the invention.

### DETAILED DESCRIPTION OF THE INVENTION

The present invention is directed to a number of sequences associated with carcinomas, especially lymphoma, breast cancer or prostate cancer. The relatively tight linkage between clonally-integrated proviruses and protooncogenes forms “provirus tagging”, in which slow-transforming retroviruses that act by an insertion mutation mechanism are used to isolate protooncogenes. In some models, uninfected animals have low cancer rates, and infected animals have high cancer rates. It is known that many of the retroviruses involved do not carry transduced host protooncogenes or pathogenic trans-acting viral genes, and thus the cancer incidence must therefor be a direct consequence of proviral integration effects into host protooncogenes. Since proviral integration is random, rare integrants will “activate” host protooncogenes that provide a selective growth advantage, and these rare events result in new proviruses at clonal stoichiometries in tumors.

The use of oncogenic retroviruses, whose sequences insert into the genome of the host organism resulting in carcinoma, allows the identification of host sequences involved in carcinoma. These sequences may then be used in a number of different ways, including diagnosis, prognosis, screening for modulators (including both agonists and antagonists), antibody generation (for immunotherapy and imaging), etc. However, as will be appreciated by those in the art, oncogenes that are identified in one type of cancer such as lymphoma or leukemia have a strong likelihood of being involved in other types of cancers as well. Thus, while the sequences outlined herein are initially identified as correlated with lymphoma, they can also be found in other types of cancers as well, outlined below.

Accordingly, the present invention provides nucleic acid and protein sequences that are associated with carcinoma, herein termed “carcinoma associated” or “CA” sequences. In a preferred embodiment, the present invention provides nucleic acid and protein sequences that are associated with carcinomas which originate in lymphatic tissue, herein termed “lymphoma associated”, “leukemia associated” or “LA” sequences.

Suitable cancers which can be diagnosed or screened for using the methods of the present invention include cancers classified by site or by histological type. Cancers classified by site include cancer of the oral cavity and pharynx (lip, tongue, salivary gland, floor of mouth, gum and other mouth, nasopharynx, tonsil, oropharynx, hypopharynx, other oral/pharynx); cancers of the digestive system (esophagus; stomach; small intestine; colon and rectum; anus, anal canal, and anorectum; liver; intrahepatic bile duct; gallbladder; other biliary; pancreas; retroperitoneum; peritoneum, omentum, and mesentery; other digestive); cancers of the respiratory system (nasal cavity, middle ear, and sinuses; larynx; lung and bronchus; pleura; trachea, mediastinum, and other respiratory); cancers of the mesothelioma; bones and joints; and soft tissue, including heart; skin cancers, including melanomas and other non-epithelial skin cancers; Kaposi's sarcoma and breast cancer; cancer of the female genital system (cervix uteri; corpus uteri; uterus, nos; ovary; vagina; vulva; and other female genital); cancers of the male genital system (prostate gland; testis; penis; and other male genital); cancers of the urinary system (urinary bladder; kidney and renal pelvis; ureter; and other urinary); cancers of the eye and orbit; cancers of the brain and nervous system (brain; and other nervous system); cancers of the endocrine system (thyroid gland and other endocrine, including thymus); cancers of the lymphomas (hodgkin's disease and non-hodgkin's lymphoma), multiple myeloma, and leukemias (lymphocytic leukemia; myeloid leukemia; monocytic leukemia; and other leukemias).

Other cancers, classified by histological type, that may be associated with the sequences of the invention include, but are not limited to, Neoplasm, malignant; Carcinoma, NOS; Carcinoma, undifferentiated, NOS; Giant and spindle cell carcinoma; Small cell carcinoma, NOS; Papillary carcinoma, NOS; Squamous cell carcinoma, NOS; Lymphoepithelial carcinoma; Basal cell carcinoma, NOS; Pilomatrix carcinoma; Transitional cell carcinoma, NOS; Papillary transitional cell carcinoma; Adenocarcinoma, NOS; Gastrinoma, malignant; Cholangiocarcinoma; Hepatocellular carcinoma, NOS; Combined hepatocellular carcinoma and cholangiocarcinoma; Trabecular adenocarcinoma; Adenoid cystic carcinoma; Adenocarcinoma in adenomatous polyp; Adenocarcinoma, familial polyposis coli; Solid carcinoma, NOS; Carcinoid tumor, malignant; Branchiolo-alveolar adenocarcinoma; Papillary adenocarcinoma, NOS; Chromophobe carcinoma; Acidophil carcinoma; Oxyphilic adenocarcinoma; Basophil carcinoma; Clear cell adenocarcinoma, NOS; Granular cell carcinoma; Follicular adenocarcinoma, NOS; Papillary and follicular adenocarcinoma; Nonencapsulating sclerosing carcinoma; Adrenal cortical carcinoma; Endometroid carcinoma; Skin appendage carcinoma; Apocrine adenocarcinoma; Sebaceous adenocarcinoma; Ceruminous adenocarcinoma; Mucoepidermoid carcinoma; Cystadenocarcinoma, NOS; Papillary cystadenocarcinoma, NOS; Papillary serous cystadenocarcinoma; Mucinous cystadenocarcinoma, NOS; Mucinous adenocarcinoma; Signet ring cell carcinoma; Infiltrating duct carcinoma; Medullary carcinoma, NOS; Lobular carcinoma; Inflammatory carcinoma; Paget”s disease, mammary; Acinar cell carcinoma; Adenosquamous carcinoma; Adenocarcinoma w/ squamous metaplasia; Thymoma, malignant; Ovarian stromal tumor, malignant; Thecoma, malignant; Granulosa cell tumor, malignant; Androblastoma, malignant; Sertoli cell carcinoma; Leydig cell tumor, malignant; Lipid cell tumor, malignant; Paraganglioma, malignant; Extra-mammary paraganglioma, malignant; Pheochromocytoma; Glomangiosarcoma; Malignant melanoma, NOS; Amelanotic melanoma; Superficial spreading melanoma; Malig melanoma in giant pigmented nevus; Epithelioid cell melanoma; Blue nevus, malignant; Sarcoma, NOS; Fibrosarcoma, NOS; Fibrous histiocytoma, malignant; Myxosarcoma; Liposarcoma, NOS; Leiomyosarcoma, NOS; Rhabdomyosarcoma, NOS; Embryonal rhabdomyosarcoma; Alveolar rhabdomyosarcoma; Stromal sarcoma, NOS; Mixed tumor, malignant, NOS; Mullerian mixed tumor; Nephroblastoma; Hepatoblastoma; Carcinosarcoma, NOS; Mesenchymoma, malignant; Brenner tumor, malignant; Phyllodes tumor, malignant; Synovial sarcoma, NOS; Mesothelioma, malignant; Dysgerminoma; Embryonal carcinoma, NOS; Teratoma, malignant, NOS; Struma ovarii, malignant; Choriocarcinoma; Mesonephroma, malignant; Hemangiosarcoma; Hemangioendothelioma, malignant; Kaposi's sarcoma; Hemangiopericytoma, malignant; Lymphangiosarcoma; Osteosarcoma, NOS; Juxtacortical osteosarcoma; Chondrosarcoma, NOS; Chondroblastoma, malignant; Mesenchymal chondrosarcoma; Giant cell tumor of bone; Ewing's sarcoma; Odontogenic tumor, malignant; Ameloblastic odontosarcoma; Ameloblastoma, malignant; Ameloblastic fibrosarcoma; Pinealoma, malignant; Chordoma; Glioma, malignant; Ependymoma, NOS; Astrocytoma, NOS; Protoplasmic astrocytoma; Fibrillary astrocytoma; Astroblastoma; Glioblastoma, NOS; Oligodendroglioma, NOS; Oligodendroblastoma; Primitive neuroectodermal; Cerebellar sarcoma, NOS; Ganglioneuroblastoma; Neuroblastoma, NOS; Retinoblastoma, NOS; Olfactory neurogenic tumor; Meningioma, malignant; Neurofibrosarcoma; Neurilemmoma, malignant; Granular cell tumor, malignant; Malignant lymphoma, NOS; Hodgkin's disease, NOS; Hodgkin's; paragranuloma, NOS; Malignant lymphoma, small lymphocytic; Malignant lymphoma, large cell, diffuse; Malignant lymphoma, follicular, NOS; Mycosis fungoides; Other specified non-Hodgkin's lymphomas; Malignant histiocytosis; Multiple myeloma; Mast cell sarcoma; Immunoproliferative small intestinal disease; Leukemia, NOS; Lymphoid leukemia, NOS; Plasma cell leukemia; Erythroleukemia; Lymphosarcoma cell leukemia; Myeloid leukemia, NOS; Basophilic leukemia; Eosinophilic leukemia; Monocytic leukemia, NOS; Mast cell leukemia; Megakaryoblastic leukemia; Myeloid sarcoma; and Hairy cell leukemia.

Association in this context means that the nucleotide or protein sequences are either differentially expressed, activated, inactivated or altered in carcinomas as compared to normal tissue. As outlined below, CA sequences include those that are up-regulated (i.e. expressed at a higher level), as well as those that are down-regulated (i.e. expressed at a lower level), in carcinomas. CA sequences also include sequences which have been altered (i.e., truncated sequences or sequences with substitutions, deletions or insertions, including point mutations) and show either the same expression profile or an altered profile. In a preferred embodiment, the CA sequences are from humans; however, as will be appreciated by those in the art, CA sequences from other organisms may be useful in animal models of disease and drug evaluation; thus, other CA sequences are provided, from vertebrates, including mammals, including rodents (rats, mice, hamsters, guinea pigs, etc.), primates, farm animals (including sheep, goats, pigs, cows, horses, etc). In some cases, prokaryotic CA sequences may be useful. CA sequences from other organisms may be obtained using the techniques outlined below.

CA sequences can include both nucleic acid and amino acid sequences. In a preferred embodiment, the CA sequences are recombinant nucleic acids. By the term “recombinant nucleic acid” herein is meant nucleic acid, originally formed in vitro, in general, by the manipulation of nucleic acid by polymerases and endonucleases, in a form not normally found in nature. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Similarly, a “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of an CA protein from one organism in a different organism or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed below.

In a preferred embodiment, the CA sequences are nucleic acids. As will be appreciated by those in the art and is more fully outlined below, CA sequences are useful in a variety of applications, including diagnostic applications, which will detect naturally occurring nucleic acids, as well as screening applications; for example, biochips comprising nucleic acid probes to the CA sequences can be generated. In the broadest sense, then, by “nucleic acid” or “oligonucleotide” or grammatical equivalents herein means at least two nucleotides covalently linked together. A nucleic acid of the present invention will generally contain phosphodiester bonds, although in some cases, as outlined as below (for example in antisense applications or when a candidate agent is a nucleic acid), nucleic acid analogs may be used that have alternate backbones, comprising, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meieretal., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. These modifications of the ribose-phosphate backbone may be done for a variety of reasons, for example to increase the stability and half-life of such molecules in physiological environments for use in anti-sense applications or as probes on a biochip.

As will be appreciated by those in the art, all of these nucleic acid analogs may find use in the present invention. In addition, mixtures of naturally occurring nucleic acids and analogs can be made; alternatively, mixtures of different nucleic acid analogs, and mixtures of naturally occurring nucleic acids and analogs may be made.

The nucleic acids may be single stranded or double stranded, as specified, or contain portions of both double stranded or single stranded sequence. As will be appreciated by those in the art, the depiction of a single strand “Watson” also defines the sequence of the other strand “Crick”; thus the sequences described herein also includes the complement of the sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xanthine hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

An CA sequence can be initially identified by substantial nucleic acid and/or amino acid sequence homology to the CA sequences outlined herein. Such homology can be based upon the overall nucleic acid or amino acid sequence, and is generally determined as outlined below, using either homology programs or hybridization conditions.

The CA sequences of the invention were initially identified as described herein; basically, infection of mice with murine leukemia viruses (MLV) resulted in lymphoma, although many of these sequences will also be involved in other cancers as is generally outlined herein.

The CA sequences outlined herein comprise the insertion sites for the virus. In general, the retrovirus can cause carcinomas in three basic ways: first of all, by inserting upstream of a normally silent host gene and activating it (e.g. promoter insertion); secondly, by truncating a host gene that leads to oncogenesis; or by enhancing the transcription of a neighboring gene. For example, retrovirus enhancers, including SL3-3, are known to act on genes up to approximately 200 kilobases of the insertion site.

In a preferred embodiment, CA sequences are those that are up-regulated in carcinomas; that is, the expression of these genes is higher in carcinoma tissue as compared to normal tissue of the same differentiation stage. “Up-regulation” as used herein means at least about 50%, more preferably at least about 100%, more preferably at least about 150%, more preferably, at least about 200%, with from 300 to at least 1000% being especially preferred.

In a preferred embodiment, CA sequences are those that are down-regulated in carcinomas; that is, the expression of these genes is lower in carcinoma tissue as compared to normal I tissue of the same differentiation stage. “Down-regulation” as used herein means at least about 50%, more preferably at least about 100%, more preferably at least about 150%, more preferably, at least about 200%, with from 300 to at least 1000% being especially preferred.

In a preferred embodiment, CA sequences are those that are altered but show either the same expression profile or an altered profile as compared to normal lymphoid tissue of the same differentiation stage. “Altered CA sequences” as used herein refers to sequences which are truncated, contain insertions or contain point mutations.

CA proteins of the present invention may be classified as secreted proteins, transmembrane proteins or intracellular proteins.

In a preferred embodiment the CA protein is an intracellular protein. Intracellular proteins may be found in the cytoplasm and/or in the nucleus. Intracellular proteins are involved in all aspects of cellular function and replication (including, for example, signaling pathways); aberrant expression of such proteins results in unregulated or disregulated cellular processes. For example, many intracellular proteins have enzymatic activity such as protein kinase activity, protein phosphatase activity, protease activity, nucleotide cyclase activity, polymerase activity and the like. Intracellular proteins also serve as docking proteins that are involved in organizing complexes of proteins, or targeting proteins to various subcellular localizations, and are involved in maintaining the structural integrity of organelles.

An increasingly appreciated concept in characterizing intracellular proteins is the presence in the proteins of one or more motifs for which defined functions have been attributed. In addition to the highly conserved sequences found in the enzymatic domain of proteins, highly conserved sequences have been identified in proteins that are involved in protein-protein interaction. For example, Src-homology-2 (SH2) domains bind tyrosine-phosphorylated targets in a sequence dependent manner. PTB domains, which are distinct from SH2 domains, also bind tyrosine phosphorylated targets. SH3 domains bind to proline-rich targets. In addition, PH domains, tetratricopeptide repeats and WD domains to name only a few, have been shown to mediate protein-protein interactions. Some of these may also be involved in binding to phospholipids or other second messengers. As will be appreciated by one of ordinary skill in the art, these motifs can be identified on the basis of primary sequence; thus, an analysis of the sequence of proteins may provide insight into both the enzymatic potential of the molecule and/or molecules with which the protein may associate.

In a preferred embodiment, the CA sequences are transmembrane proteins. Transmembrane proteins are molecules that span the phospholipid bilayer of a cell. They may have an intracellular domain, an extracellular domain, or both. The intracellular domains of such proteins may have a number of functions including those already described for intracellular proteins. For example, the intracellular domain may have enzymatic activity and/or may serve as a binding site for additional proteins. Frequently the intracellular domain of transmembrane proteins serves both roles. For example certain receptor tyrosine kinases have both protein kinase activity and SH2 domains. In addition, autophosphorylation of tyrosines on the receptor molecule itself, creates binding sites for additional SH2 domain containing proteins.

Transmembrane proteins may contain from one to many transmembrane domains. For example, receptor tyrosine kinases, certain cytokine receptors, receptor guanylyl cyclases and receptor serine/threonine protein kinases contain a single transmembrane domain. However, various other proteins including channels and adenylyl cyclases contain numerous transmembrane domains. Many important cell surface receptors are classified as “seven transmembrane domain” proteins, as they contain 7 membrane spanning regions. Important transmembrane protein receptors include, but are not limited to insulin receptor, insulin-like growth factor receptor, human growth hormone receptor, glucose transporters, transferrin receptor, epidermal growth factor receptor, low density lipoprotein receptor, epidermal growth factor receptor, leptin receptor, interleukin receptors, e.g. IL-1 receptor, IL-2 receptor, etc.

Characteristics of transmembrane domains include approximately 20 consecutive hydrophobic amino acids that may be followed by charged amino acids. Therefore, upon analysis of the amino acid sequence of a particular protein, the localization and number of transmembrane domains within the protein may be predicted.

The extracellular domains of transmembrane proteins are diverse; however, conserved motifs are found repeatedly among various extracellular domains. Conserved structure and/or functions have been ascribed to different extracellular motifs. For example, cytokine receptors are characterized by a cluster of cysteines and a WSXWS (W=tryptophan, S=serine, X=any amino acid) motif. Immunoglobulin-like domains are highly conserved. Mucin-like domains may be involved in cell adhesion and leucine-rich repeats participate in protein-protein interactions.

Many extracellular domains are involved in binding to other molecules. In one aspect, extracellular domains are receptors. Factors that bind the receptor domain include circulating ligands, which may be peptides, proteins, or small molecules such as adenosine and the like. For example, growth factors such as EGF, FGF and PDGF are circulating growth factors that bind to their cognate receptors to initiate a variety of cellular responses. Other factors include cytokines, mitogenic factors, neurotrophic factors and the like. Extracellular domains also bind to cell-associated molecules. In this respect, they mediate cell-cell interactions. Cell-associated ligands can be tethered to the cell for example via a glycosylphosphatidylinositol (GPI) anchor, or may themselves be transmembrane proteins. Extracellular domains also associate with the extracellular matrix and contribute to the maintenance of the cell structure.

CA proteins that are transmembrane are particularly preferred in the present invention as they are good targets for immunotherapeutics, as are described herein. In addition, as outlined below, transmembrane proteins can be also useful in imaging modalities.

It will also be appreciated by those in the art that a transmembrane protein can be made soluble by removing transmembrane sequences, for example through recombinant methods. Furthermore, transmembrane proteins that have been made soluble can be made to be secreted through recombinant means by adding an appropriate signal sequence.

In a preferred embodiment, the CA proteins are secreted proteins; the secretion of which can be either constitutive or regulated. These proteins have a signal peptide or signal sequence that targets the molecule to the secretory pathway. Secreted proteins are involved in numerous physiological events; by virtue of their circulating nature, they serve to transmit signals to various other cell types. The secreted protein may function in an autocrine manner (acting on the cell that secreted the factor), a paracrine manner (acting on cells in close proximity to the cell that secreted the factor) or an endocrine manner (acting on cells at a distance). Thus secreted molecules find use in modulating or altering numerous aspects of physiology. CA proteins that are secreted proteins are particularly preferred in the present invention as they serve as good targets for diagnostic markers, for example for blood tests.

An CA sequence is initially identified by substantial nucleic acid and/or amino acid sequence homology to the CA sequences outlined herein. Such homology can be based upon the overall nucleic acid or amino acid sequence, and is generally determined as outlined below, using either homology programs or hybridization conditions.

As used herein, a nucleic acid is a “CA nucleic acid” if the overall homology of the nucleic acid sequence to one of the nucleic acids of Tables 1-10 is preferably greater than about 75%, more preferably greater than about 80%, even more preferably greater than about 85% and most preferably greater than 90%. In some embodiments the homology will be as high as about 93 to 95 or 98%. In a preferred embodiment, the sequences which are used to determine sequence identity or similarity are selected from those of the nucleic acids of Tables 1-10. In another embodiment, the sequences are naturally occurring allelic variants of the sequences of the nucleic acids of Tables 1-10. In another embodiment, the sequences are sequence variants as further described herein.

Homology in this context means sequence similarity or identity, with identity being preferred. A preferred comparison for homology purposes is to compare the sequence containing sequencing errors to the correct sequence. This homology will be determined using standard techniques known in the art, including, but not limited to, the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387-395 (1984), preferably using the default settings, or by inspection.

One example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989). Useful PILEUP parameters including a default gap weight of 3.00, a default gap length weight of 0.10, and weighted end gaps.

Another example of a useful algorithm is the BLAST algorithm, described in Altschul et al., J. Mol. Biol. 215, 403-410, (1990) and Karlin et al., PNAS USA 90:5873-5787 (1993). A particularly useful BLAST program is the WU-BLAST-2 program which was obtained from Altschul et al., Methods in Enzymology, 266: 460-480 (1996); http://blast.wustl]. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

Thus, “percent (%) nucleic acid sequence identity” is defined as the percentage of nucleotide residues in a candidate sequence that are identical with the nucleotide residues of the nucleic acids of Tables 1-10. A preferred method utilizes the BLASTN module of WU-BLAST-2 set to the default parameters, with overlap span and overlap fraction set to 1 and 0.125, respectively.

The alignment may include the introduction of gaps in the sequences to be aligned. In addition, for sequences which contain either more or fewer nucleotides than those of the nucleic acids of Tables 1-10, it is understood that the percentage of homology will be determined based on the number of homologous nucleosides in relation to the total number of nucleosides. Thus, for example, homology of sequences shorter than those of the sequences identified herein and as discussed below, will be determined using the number of nucleosides in the shorter sequence.

In one embodiment, the nucleic acid homology is determined through hybridization studies. Thus, for example, nucleic acids which hybridize under high stringency to the nucleic acids identified in the figures, or their complements, are considered CA sequences. High stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide.

In another embodiment, less stringent hybridization conditions are used; for example, moderate or low stringency conditions may be used, as are known in the art; see Maniatis and Ausubel, supra, and Tijssen, supra.

In addition, the CA nucleic acid sequences of the invention are fragments of larger genes, i.e. they are nucleic acid segments. Alternatively, the CA nucleic acid sequences can serve as indicators of oncogene position, for example, the CA sequence may be an enhancer that activates a protooncogene. “Genes” in this context includes coding regions, non-coding regions, and mixtures of coding and non-coding regions. Accordingly, as will be appreciated by those in the art, using the sequences provided herein, additional sequences of the CA genes can be obtained, using techniques well known in the art for cloning either longer sequences or the full length sequences; see Maniatis et al., and Ausubel, et al., supra, hereby expressly incorporated by reference. In general, this is done using PCR, for example, kinetic PCR.

Once the CA nucleic acid is identified, it can be cloned and, if necessary, its constituent parts recombined to form the entire CA nucleic acid. Once isolated from its natural source, e.g., contained within a plasmid or other vector or excised therefrom as a linear nucleic acid segment, the recombinant CA nucleic acid can be further used as a probe to identify and isolate other CA nucleic acids, for example additional coding regions. It can also be used as a “precursor” nucleic acid to make modified or variant CA nucleic acids and proteins.

The CA nucleic acids of the present invention are used in several ways. In a first embodiment, nucleic acid probes to the CA nucleic acids are made and attached to biochips to be used in screening and diagnostic methods, as outlined below, or for administration, for example for gene therapy and/or antisense applications. Alternatively, the CA nucleic acids that include coding regions of CA proteins can be put into expression vectors for the expression of CA proteins, again either for screening purposes or for administration to a patient.

In a preferred embodiment, nucleic acid probes to CA nucleic acids (both the nucleic acid sequences outlined in the figures and/or the complements thereof) are made. The nucleic acid probes attached to the biochip are designed to be substantially complementary to the CA nucleic acids, i.e. the target sequence (either the target sequence of the sample or to other probe sequences, for example in sandwich assays), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions, particularly high stringency conditions, as outlined herein.

A nucleic acid probe is generally single stranded but can be partially single and partially double stranded. The strandedness of the probe is dictated by the structure, composition, and properties of the target sequence. In general, the nucleic acid probes range from about 8 to about 100 bases long, with from about 10 to about 80 bases being preferred, and from about 30 to about 50 bases being particularly preferred. That is, generally whole genes are not used. In some embodiments, much longer nucleic acids can be used, up to hundreds of bases.

In a preferred embodiment, more than one probe per sequence is used, with either overlapping probes or probes to different sections of the target being used. That is, two, three, four or more probes, with three being preferred, are used to build in a redundancy for a particular target. The probes can be overlapping (i.e. have some sequence in common), or separate.

As will be appreciated by those in the art, nucleic acids can be attached or immobilized to a solid support in a wide variety of ways. By “immobilized” and grammatical equivalents herein is meant the association or binding between the nucleic acid probe and the solid support is sufficient to be stable under the conditions of binding, washing, analysis, and removal as outlined below. The binding can be covalent or non-covalent. By “non-covalent binding” and grammatical equivalents herein is meant one or more of either electrostatic, hydrophilic, and hydrophobic interactions. Included in non-covalent binding is the covalent attachment of a molecule, such as, streptavidin to the support and the non-covalent binding of the biotinylated probe to the streptavidin. By “covalent binding” and grammatical equivalents herein is meant that the two moieties, the solid support and the probe, are attached by at least one bond, including sigma bonds, pi bonds and coordination bonds. Covalent bonds can be formed directly between the probe and the solid support or can be formed by a cross linker or by inclusion of a specific reactive group on either the solid support or the probe or both molecules. Immobilization may also involve a combination of covalent and non-covalent interactions.

In general, the probes are attached to the biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant any material that can be modified to contain discrete individual sites appropriate for the attachment or association of the nucleic acid probes and is amenable to at least one detection method. As will be appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, resins, silica or silica-based materials including silicon and modified silicon, carbon, metals, inorganic glasses, etc. In general, the substrates allow optical detection and do not appreciably fluoresce.

In a preferred embodiment, the surface of the biochip and the probe may be derivatized with chemical functional groups for subsequent attachment of the two. Thus, for example, the biochip is derivatized with a chemical functional group including, but not limited to, amino groups, carboxy groups, oxo groups and thiol groups, with amino groups being particularly preferred. Using these functional groups, the probes can be attached using functional groups on the probes. For example, nucleic acids containing amino groups can be attached to surfaces comprising amino groups, for example using linkers as are known in the art; for example, homo-or hetero-bifunctional linkers as are well known (see 1994 Pierce Chemical Company catalog, technical section on cross-linkers, pages 155-200, incorporated herein by reference). In addition, in some cases, additional linkers, such as alkyl groups (including substituted and heteroalkyl groups) may be used.

In this embodiment, the oligonucleotides are synthesized as is known in the art, and then attached to the surface of the solid support. As will be appreciated by those skilled in the art, either the 5′ or 3′ terminus may be attached to the solid support, or attachment may be via an internal nucleoside.

In an additional embodiment, the immobilization to the solid support may be very strong, yet non-covalent. For example, biotinylated oligonucleotides can be made, which bind to surfaces covalently coated with streptavidin, resulting in attachment.

Alternatively, the oligonucleotides may be synthesized on the surface, as is known in the art. For example, photoactivation techniques utilizing photopolymerization compounds and techniques are used. In a preferred embodiment, the nucleic acids can be synthesized in situ, using well known photolithographic techniques, such as those described in WO 95/25116; WO 95/35505; U.S. Pat. Nos. 5,700,637 and 5,445,934; and references cited within, all of which are expressly incorporated by reference; these methods of attachment form the basis of the Affymetrix GeneChip technology.

In addition to the solid-phase technology represented by biochip arrays, gene expression can also be quantified using liquid-phase arrays. One such system is kinetic polymerase chain reaction (PCR). Kinetic PCR allows for the simultaneous amplification and quantification of specific nucleic acid sequences. The specificity is derived from synthetic oligonucleotide primers designed to preferentially adhere to single-stranded nucleic acid sequences bracketing the target site. This pair of oligonucleotide primers form specific, non-covalently bound complexes on each strand of the target sequence. These complexes facilitate in vitro transcription of double-stranded DNA in opposite orientations. Temperature cycling of the reaction mixture creates a continuous cycle of primer binding, transcription, and re-melting of the nucleic acid to individual strands. The result is an exponential increase of the target dsDNA product. This product can be quantified in real time either through the use of an intercalating dye or a sequence specific probe. SYBR® Greene I, is an example of an intercalating dye, that preferentially binds to dsDNA resulting in a concomitant increase in the fluorescent signal. Sequence specific probes, such as used with TaqMan® technology, consist of a fluorochrome and a quenching molecule covalently bound to opposite ends of an oligonucleotide. The probe is designed to selectively bind the target DNA sequence between the two primers. When the DNA strands are synthesized during the PCR reaction, the fluorochrome is cleaved from the probe by the exonuclease activity of the polymerase resulting in signal dequenching. The probe signaling method can be more specific than the intercalating dye method, but in each case, signal strength is proportional to the dsDNA product produced. Each type of quantification method can be used in multi-well liquid phase arrays with each well representing primers and/or probes specific to nucleic acid sequences of interest. When used with messenger RNA preparations of tissues or cell lines, and an array of probe/primer reactions can simultaneously quantify the expression of multiple gene products of interest. See Germer, S., et al., Genome Res. 10:258-266 (2000); Held, C. A., et al., Genome Res. 6, 986-994 (1996).

In a preferred embodiment, CA nucleic acids encoding CA proteins are used to make a variety of expression vectors to express CA proteins which can then be used in screening assays, as described below. The expression vectors may be either self-replicating extrachromosomal vectors or vectors which integrate into a host genome. Generally, these expression vectors include transcriptional and translational regulatory nucleic acid operably linked to the nucleic acid encoding the CA protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, synthetic oligonucleotide adaptors or linkers are used in accordance with conventional practice. The transcriptional and translational regulatory nucleic acid will generally be appropriate to the host cell used to express the CA protein; for example, transcriptional and translational regulatory nucleic acid sequences from Bacillus are preferably used to express the CA protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a procaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used.

The CA proteins of the present invention are produced by culturing a host cell transformed with an expression vector containing nucleic acid encoding an CA protein, under the appropriate conditions to induce or cause expression of the CA protein. The conditions appropriate for CA protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fungi, and insect, plant and animal cells, including mammalian cells. Of particular interest are Drosophila melanogaster cells, Saccharomyces cerevisiae and other yeasts, E coli, Bacillus subtilis, Sf9 cells, C129 cells, 293 cells, Neurospora, BHK, CHO, COS, HeLa cells, THP1 cell line (a macrophage cell line) and human cells and cell lines.

In a preferred embodiment, the CA proteins are expressed in mammalian cells. Mammalian expression systems are also known in the art, and include retroviral systems. A preferred expression vector system is a retroviral vector system such as is generally described in PCT/US97/01019 and PCT/US97101048, both of which are hereby expressly incorporated by reference. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter. Typically, transcription termination and polyadenylation sequences recognized by mammalian cells are regulatory regions located 3′ to the translation stop codon and thus, together with the promoter elements, flank the coding sequence. Examples of transcription terminator and polyadenlytion signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, CA proteins are expressed in bacterial systems. Bacterial expression systems are well known in the art. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription. In addition to a functioning promoter sequence, an efficient ribosome binding site is desirable. The expression vector may also include a signal peptide sequence that provides for secretion of the CA protein in bacteria. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others. The bacterial expression vectors are transformed into bacterial host cells using techniques well known in the art, such as calcium chloride treatment, electroporation, and others.

In one embodiment, CA proteins are produced in insect cells. Expression vectors for the transformation of insect cells, and in particular, baculovirus-based expression vectors, are well known in the art.

In a preferred embodiment, CA protein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica.

The CA protein may also be made as a fusion protein, using techniques well known in the art. Thus, for example, for the creation of monoclonal antibodies. If the desired epitope is small, the CA protein may be fused to a carrier protein to form an immunogen. Alternatively, the CA protein may be made as a fusion protein to increase expression, or for other reasons. For example, when the CA protein is an CA peptide, the nucleic acid encoding the peptide may be linked to other nucleic acid for expression purposes.

In one embodiment, the CA nucleic acids, proteins and antibodies of the invention are labeled. By “labeled” herein is meant that a compound has at least one element, isotope or chemical compound attached to enable the detection of the compound. In general, labels fall into three classes: a) isotopic labels, which may be radioactive or heavy isotopes; b) immune labels, which may be antibodies or antigens; and c) colored or fluorescent dyes. The labels may be incorporated into the CA nucleic acids, proteins and antibodies at any position. For example, the label should be capable of producing, either directly or indirectly, a detectable signal. The detectable moiety may be a radioisotope, such as ³H, ¹⁴C, ³²P, ³⁵S, or ¹²⁵I a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the label may be employed, including those methods described by Hunter et al., Nature, 144:945 9(1962); David et al., Biochemistry, 13:1014 (1974); Pain et al., J. Immunol. Meth., 40:219 (1981); and Nygren, J. Histochem. and Cytochem., 30:407 (1982).

Accordingly, the present invention also provides CA protein sequences. An CA protein of the present invention may be identified in several ways. “Protein” in this sense includes proteins, polypeptides, and peptides. As will be appreciated by those in the art, the nucleic acid sequences of the invention can be used to generate protein sequences. There are a variety of ways to do this, including cloning the entire gene and verifying its frame and amino acid sequence, or by comparing it to known sequences to search for homology to provide a frame, assuming the CA protein has homology to some protein in the database being used. Generally, the nucleic acid sequences are input into a program that will search all three frames for homology. This is done in a preferred embodiment using the following NCBI Advanced BLAST parameters. The program is blastx or blastn. The database is nr. The input data is as “Sequence in FASTA format”. The organism list is “none”. The “expect” is 10; the filter is default. The “descriptions” is 500, the “alignments” is 500, and the “alignment view” is pairwise. The “query Genetic Codes” is standard (1). The matrix is BLOSUM62; gap existence cost is 11, per residue gap cost is 1; and the lambda ratio is 0.85 default. This results in the generation of a putative protein sequence.

Also included within one embodiment of CA proteins are amino acid variants of the naturally occurring sequences, as determined herein. Preferably, the variants are preferably greater than about 75% homologous to the wild-type sequence, more preferably greater than about 80%, even more preferably greater than about 85% and most preferably greater than 90%. In some embodiments the homology will be as high as about 93 to 95 or 98%. As for nucleic acids, homology in this context means sequence similarity or identity, with identity being preferred. This homology will be determined using standard techniques known in the art as are outlined above for the nucleic acid homologies.

CA proteins of the present invention may be shorter or longer than the wild type amino acid sequences. Thus, in a preferred embodiment, included within the definition of CA proteins are portions or fragments of the wild type sequences herein. In addition, as outlined above, the CA nucleic acids of the invention may be used to obtain additional coding regions, and thus additional protein sequence, using techniques known in the art.

In a preferred embodiment, the CA proteins are derivative or variant CA proteins as compared to the wild-type sequence. That is, as outlined more fully below, the derivative CA peptide will contain at least one amino acid substitution, deletion or insertion, with amino acid substitutions being particularly preferred. The amino acid substitution, insertion or deletion may occur at any residue within the CA peptide.

Also included in an embodiment of CA proteins of the present invention are amino acid sequence variants. These variants fall into one or more of three classes: substitutional, insertional or deletional variants. These variants ordinarily are prepared by site specific mutagenesis of nucleotides in the DNA encoding the CA protein, using cassette or PCR mutagenesis or other techniques well known in the art, to produce DNA encoding the variant, and thereafter expressing the DNA in recombinant cell culture as outlined above. However, variant CA protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the CA protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed CA variants screened for the optimal combination of desired activity. Techniques for making substitution mutations at predetermined sites in DNA having a known sequence are well known, for example, M13 primer mutagenesis and LAR mutagenesis. Screening of the mutants is done using assays of CA protein activities.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the CA protein are desired, substitutions are generally made in accordance with the following chart:

CHART I

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Chart I. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

The variants typically exhibit the same qualitative biological activity and will elicit the same immune response as the naturally-occurring analogue, although variants also are selected to modify the characteristics of the CA proteins as needed. Alternatively, the variant may be designed such that the biological activity of the CA protein is altered. For example, glycosylation sites may be altered or removed, dominant negative mutations created, etc.

Covalent modifications of CA polypeptides are included within the scope of this invention, for example for use in screening. One type of covalent modification includes reacting targeted amino acid residues of an CA polypeptide with an organic derivatizing agent that is capable of reacting with selected side chains or the N-or C-terminal residues of an CA polypeptide. Derivatization with bifunctional agents is useful, for instance, for crosslinking CA polypeptides to a water-insoluble support matrix or surface for use in the method for purifying anti-CA antibodies or screening assays, as is more fully described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl, threonyl or tyrosyl residues, methylation of the a-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Another type of covalent modification of the CA polypeptide included within the scope of this invention comprises altering the native glycosylation pattern of the polypeptide. “Altering the native glycosylation pattern” is intended for purposes herein to mean deleting one or more carbohydrate moieties found in native sequence CA polypeptide, and/or adding one or more glycosylation sites that are not present in the native sequence CA polypeptide.

Addition of glycosylation sites to CA polypeptides may be accomplished by altering the amino acid sequence thereof. The alteration may be made, for example, by the addition of, or substitution by, one or more serine or threonine residues to the native sequence CA polypeptide (for O-linked glycosylation sites). The CA amino acid sequence may optionally be altered through changes at the DNA level, particularly by mutating the DNA encoding the CA polypeptide at preselected bases such that codons are generated that will translate into the desired amino acids.

Another means of increasing the number of carbohydrate moieties on the CA polypeptide is by chemical or enzymatic coupling of glycosides to the polypeptide. Such methods are described in the art, e.g., in WO 87/05330 published Sep. 11, 1987, and in Aplin and Wriston, LA Crit. Rev. Biochem., pp. 259-306 (1981).

Removal of carbohydrate moieties present on the CA polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131(1981). Enzymatic cleavage of carbohydrate moieties on polypeptides can be achieved by the use of a variety of endo-and exo-glycosidases as described by Thotakura et al., Meth. Enzymol., 138:350 (1987).

Another type of covalent modification of CA comprises linking the CA polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

CA polypeptides of the present invention may also be modified in a way to form chimeric molecules comprising an CA polypeptide fused to another, heterologous polypeptide or amino acid sequence. In one embodiment, such a chimeric molecule comprises a fusion of an CA polypeptide with a tag polypeptide which provides an epitope to which an anti-tag antibody can selectively bind. The epitope tag is generally placed at the amino-or carboxyl-terminus of the CA polypeptide, although internal fusions may also be tolerated in some instances. The presence of such epitope-tagged forms of an CA polypeptide can be detected using an antibody against the tag polypeptide. Also, provision of the epitope tag enables the CA polypeptide to be readily purified by affinity purification using an anti-tag antibody or another type of affinity matrix that binds to the epitope tag. In an alternative embodiment, the chimeric molecule may comprise a fusion of an CA polypeptide with an immunoglobulin or a particular region of an immunoglobulin. For a bivalent form of the chimeric molecule, such a fusion could be to the Fc region of an IgG molecule.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

Also included with the definition of CA protein in one embodiment are other CA proteins of the CA family, and CA proteins from other organisms, which are cloned and expressed as outlined below. Thus, probe or degenerate polymerase chain reaction (PCR) primer sequences may be used to find other related CA proteins from humans or other organisms. As will be appreciated by those in the art, particularly useful probe and/or PCR primer sequences include the unique areas of the CA nucleic acid sequence. As is generally known in the art, preferred PCR primers are from about 15 to about 35 nucleotides in length, with from about 20 to about 30 being preferred, and may contain inosine as needed. The conditions for the PCR reaction are well known in the art.

In addition, as is outlined herein, CA proteins can be made that are longer than those encoded by the nucleic acids of the figures, for example, by the elucidation of additional sequences, the addition of epitope or purification tags, the addition of other fusion sequences, etc.

CA proteins may also be identified as being encoded by CA nucleic acids. Thus, CA proteins are encoded by nucleic acids that will hybridize to the sequences of the sequence listings, or their complements, as outlined herein.

In a preferred embodiment, the invention provides CA antibodies. In a preferred embodiment, when the CA protein is to be used to generate antibodies, for example for immunotherapy, the CA protein should share at least one epitope or determinant with the full length protein. By “epitope” or “determinant” herein is meant a portion of a protein which will generate and/or bind an antibody or T-cell receptor in the context of MHC. Thus, in most instances, antibodies made to a smaller CA protein will be able to bind to the full length protein. In a preferred embodiment, the epitope is unique; that is, antibodies generated to a unique epitope show little or no cross-reactivity.

In one embodiment, the term “antibody” includes antibody fragments, as are known in the art, including Fab, Fab₂, single chain antibodies (Fv for example), chimeric antibodies, etc., either produced by the modification of whole antibodies or those synthesized de novo using recombinant DNA technologies.

Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid of the figures or fragment thereof or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler and Milstein, Nature, 256:495 (1975).

In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a polypeptide encoded by a nucleic acid of Tables 1-10, or fragment thereof or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In one embodiment, the antibodies are bispecific antibodies. Bispecific antibodies are monoclonal, preferably human or humanized, antibodies that have binding specificities for at least two different antigens. In the present case, one of the binding specificities is for a protein encoded by a nucleic acid of Tables 1, or a fragment thereof, the other one is for any other antigen, and preferably for a cell-surface protein or receptor or receptor subunit, preferably one that is tumor specific.

In a preferred embodiment, the antibodies to CA are capable of reducing or eliminating the biological function of CA, as is described below. That is, the addition of anti-CA antibodies (either polyclonal or preferably monoclonal) to CA (or cells containing CA) may reduce or eliminate the CA activity. Generally, at least a 25% decrease in activity is preferred, with at least about 50% being particularly preferred and about a 95-100% decrease being especially preferred.

In a preferred embodiment the antibodies to the CA proteins are humanized antibodies. Humanized forms of non-human (e.g., murine) antibodies are chimeric molecules of immunoglobulins, immunoglobulin chains or fragments thereof (such as Fv, Fab, Fab′, F(ab′)₂ or other antigen binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework residues (FR) regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies [Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

By immunotherapy is meant treatment of a carcinoma with an antibody raised against an CA protein. As used herein, immunotherapy can be passive or active. Passive immunotherapy as defined herein is the passive transfer of antibody to a recipient (patient). Active immunization is the induction of antibody and/or T-cell responses in a recipient (patient). Induction of an immune response is the result of providing the recipient with an antigen to which antibodies are raised. As appreciated by one of ordinary skill in the art, the antigen may be provided by injecting a polypeptide against which antibodies are desired to be raised into a recipient, or contacting the recipient with a nucleic acid capable of expressing the antigen and under conditions for expression of the antigen.

In a preferred embodiment, oncogenes which encode secreted growth factors may be inhibited by raising antibodies against CA proteins that are secreted proteins as described above. Without being bound by theory, antibodies used for treatment, bind and prevent the secreted protein from binding to its receptor, thereby inactivating the secreted CA protein.

In another preferred embodiment, the CA protein to which antibodies are raised is a transmembrane protein. Without being bound by theory, antibodies used for treatment, bind the extracellular domain of the CA protein and prevent it from binding to other proteins, such as circulating ligands or cell-associated molecules. The antibody may cause down-regulation of the transmembrane CA protein.

As will be appreciated by one of ordinary skill in the art, the antibody may be a competitive, non-competitive or uncompetitive inhibitor of protein binding to the extracellular domain of the CA protein. The antibody is also an antagonist of the CA protein. Further, the antibody prevents activation of the transmembrane CA protein. In one aspect, when the antibody prevents the binding of other molecules to the CA protein, the antibody prevents growth of the cell. The antibody may also sensitize the cell to cytotoxic agents, including, but not limited to TNF-α, TNF-β, IL-1, INF-γ and IL-2, or chemotherapeutic agents including 5FU, vinblastine, actinomycin D, cisplatin, methotrexate, and the like. In some instances the antibody belongs to a sub-type that activates serum complement when complexed with the transmembrane protein thereby mediating cytotoxicity. Thus, carcinomas may be treated by administering to a patient antibodies directed against the transmembrane CA protein.

In another preferred embodiment, the antibody is conjugated to a therapeutic moiety. In one aspect the therapeutic moiety is a small molecule that modulates the activity of the CA protein. In another aspect the therapeutic moiety modulates the activity of molecules associated with or in close proximity to the CA protein. The therapeutic moiety may inhibit enzymatic activity such as protease or protein kinase activity associated with carcinoma.

In a preferred embodiment, the therapeutic moiety may also be a cytotoxic agent. In this method, targeting the cytotoxic agent to tumor tissue or cells, results in a reduction in the number of afflicted cells, thereby reducing symptoms associated with carcinomas, including lymphoma. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin and the like. Cytotoxic agents also include radiochemicals made by conjugating radioisotopes to antibodies raised against CA proteins, or binding of a radionuclide to a chelating agent that has been covalently attached to the antibody. Targeting the therapeutic moiety to transmembrane CA proteins not only serves to increase the local concentration of therapeutic moiety in the carcinoma of interest, i.e., lymphoma, but also serves to reduce deleterious side effects that may be associated with the therapeutic moiety.

In another preferred embodiment, the CA protein against which the antibodies are raised is an intracellular protein. In this case, the antibody may be conjugated to a protein which facilitates entry into the cell. In one case, the antibody enters the cell by endocytosis. In another embodiment, a nucleic acid encoding the antibody is administered to the individual or cell. Moreover, wherein the CA protein can be targeted within a cell, i.e., the nucleus, an antibody thereto contains a signal for that target localization, i.e., a nuclear localization signal.

The CA antibodies of the invention specifically bind to CA proteins. By “specifically bind” herein is meant that the antibodies bind to the protein with a binding constant in the range of at least 10⁻⁴-10⁻⁶ M⁻¹, with a preferred range being 10⁻⁷-10⁻⁹ M⁻¹.

In a preferred embodiment, the CA protein is purified or isolated after expression. CA proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the CA protein may be purified using a standard anti-CA antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the CA protein. In some instances no purification will be necessary.

Once expressed and purified if necessary, the CA proteins and nucleic acids are useful in a number of applications.

In one aspect, the expression levels of genes are determined for different cellular states in the carcinoma phenotype; that is, the expression levels of genes in normal tissue and in carcinoma tissue (and in some cases, for varying severities of lymphoma that relate to prognosis, as outlined below) are evaluated to provide expression profiles. An expression profile of a particular cell state or point of development is essentially a “fingerprint” of the state; while two states may have any particular gene similarly expressed, the evaluation of a number of genes simultaneously allows the generation of a gene expression profile that is unique to the state of the cell. By comparing expression profiles of cells in different states, information regarding which genes are important (including both up- and down-regulation of genes) in each of these states is obtained. Then, diagnosis may be done or confirmed: does tissue from a particular patient have the gene expression profile of normal or carcinoma tissue.

“Differential expression,” or grammatical equivalents as used herein, refers to both qualitative as well as quantitative differences in the genes temporal and/or cellular expression patterns within and among the cells. Thus, a differentially expressed gene can qualitatively have its expression altered, including an activation or inactivation, in, for example, normal versus carcinoma tissue. That is, genes may be turned on or turned off in a particular state, relative to another state. As is apparent to the skilled artisan, any comparison of two or more states can be made. Such a qualitatively regulated gene will exhibit an expression pattern within a state or cell type which is detectable by standard techniques in one such state or cell type, but is not detectable in both. Alternatively, the determination is quantitative in that expression is increased or decreased; that is, the expression of the gene is either upregulated, resulting in an increased amount of transcript, or downregulated, resulting in a decreased amount of transcript. The degree to which expression differs need only be large enough to quantify via standard characterization techniques as outlined below, such as by use of Affymetrix GeneChip® expression arrays, Lockhart, Nature Biotechnology, 14:1675-1680 (1996), hereby expressly incorporated by reference. Other techniques include, but are not limited to, quantitative reverse transcriptase PCR, Northern analysis and RNase protection. As outlined above, preferably the change in expression (i.e. upregulation or downregulation) is at least about 50%, more preferably at least about 100%, more preferably at least about 150%, more preferably, at least about 200%, with from 300 to at least 1000% being especially preferred.

As will be appreciated by those in the art, this may be done by evaluation at either the gene transcript, or the protein level; that is, the amount of gene expression may be monitored using nucleic acid probes to the DNA or RNA equivalent of the gene transcript, and the quantification of gene expression levels, or, alternatively, the final gene product itself (protein) can be monitored, for example through the use of antibodies to the CA protein and standard immunoassays (ELISAs, etc.) or other techniques, including mass spectroscopy assays, 2D gel electrophoresis assays, etc. Thus, the proteins corresponding to CA genes, i.e. those identified as being important in a particular carcinoma phenotype, i.e., lymphoma, can be evaluated in a diagnostic test specific for that carcinoma.

In a preferred embodiment, gene expression monitoring is done and a number of genes, i.e. an expression profile, is monitored simultaneously, although multiple protein expression monitoring can be done as well. Similarly, these assays may be done on an individual basis as well.

In this embodiment, the CA nucleic acid probes may be attached to biochips as outlined herein for the detection and quantification of CA sequences in a particular cell. The assays are done as is known in the art. As will be appreciated by those in the art, any number of different CA sequences may be used as probes, with single sequence assays being used in some cases, and a plurality of the sequences described herein being used in other embodiments. In addition, while solid-phase assays are described, any number of solution based assays may be done as well.

In a preferred embodiment, both solid and solution based assays may be used to detect CA sequences that are up-regulated or down-regulated in carcinomas as compared to normal tissue. In instances where the CA sequence has been altered but shows the same expression profile or an altered expression profile, the protein will be detected as outlined herein.

In a preferred embodiment nucleic acids encoding the CA protein are detected. Although DNA or RNA encoding the CA protein may be detected, of particular interest are methods wherein the mRNA encoding a CA protein is detected. The presence of mRNA in a sample is an indication that the CA gene has been transcribed to form the mRNA, and suggests that the protein is expressed. Probes to detect the mRNA can be any nucleotide/deoxynucleotide probe that is complementary to and base pairs with the mRNA and includes but is not limited to oligonucleotides, cDNA or RNA. Probes also should contain a detectable label, as defined herein. In one method the mRNA is detected after immobilizing the nucleic acid to be examined on a solid support such as nylon membranes and hybridizing the probe with the sample. Following washing to remove the non-specifically bound probe, the label is detected. In another method detection of the mRNA is performed in situ. In this method permeabilized cells or tissue samples are contacted with a detectably labeled nucleic acid probe for sufficient time to allow the probe to hybridize with the target mRNA. Following washing to remove the non-specifically bound probe, the label is detected. For example a digoxygenin labeled riboprobe (RNA probe) that is complementary to the mRNA encoding a CA protein is detected by binding the digoxygenin with an anti-digoxygenin secondary antibody and developed with nitro blue tetrazolium and 5-bromo-4-chloro-3-indoyl phosphate.

In a preferred embodiment, any of the three classes of proteins as described herein (secreted, transmembrane or intracellular proteins) are used in diagnostic assays. The CA proteins, antibodies, nucleic acids, modified proteins and cells containing CA sequences are used in diagnostic assays. This can be done on an individual gene or corresponding polypeptide level, or as sets of assays.

As described and defined herein, CA proteins find use as markers of carcinomas, including lymphomas such as, but not limited to, Hodgkin's and non-Hodgkin lymphoma. Detection of these proteins in putative carcinoma tissue or patients allows for a determination or diagnosis of the type of carcinoma. Numerous methods known to those of ordinary skill in the art find use in detecting carcinomas. In one embodiment, antibodies are used to detect CA proteins. A preferred method separates proteins from a sample or patient by electrophoresis on a gel (typically a denaturing and reducing protein gel, but may be any other type of gel including isoelectric focusing gels and the like). Following separation of proteins, the CA protein is detected by immunoblotting with antibodies raised against the CA protein. Methods of immunoblotting are well known to those of ordinary skill in the art.

In another preferred method, antibodies to the CA protein find use in in situ imaging techniques. In this method cells are contacted with from one to many antibodies to the CA protein(s). Following washing to remove non-specific antibody binding, the presence of the antibody or antibodies is detected. In one embodiment the antibody is detected by incubating with a secondary antibody that contains a detectable label. In another method the primary antibody to the CA protein(s) contains a detectable label. In another preferred embodiment each one of multiple primary antibodies contains a distinct and detectable label. This method finds particular use in simultaneous screening for a plurality of CA proteins. As will be appreciated by one of ordinary skill in the art, numerous other histological imaging techniques are useful in the invention.

In a preferred embodiment the label is detected in a fluorometer which has the ability to detect and distinguish emissions of different wavelengths. In addition, a fluorescence activated cell sorter (FACS) can be used in the method.

In another preferred embodiment, antibodies find use in diagnosing carcinomas from blood samples. As previously described, certain CA proteins are secreted/circulating molecules. Blood samples, therefore, are useful as samples to be probed or tested for the presence of secreted CA proteins. Antibodies can be used to detect the CA proteins by any of the previously described immunoassay techniques including ELISA, immunoblotting (Western blotting), immunoprecipitation, BIACORE technology and the like, as will be appreciated by one of ordinary skill in the art.

In a preferred embodiment, in situ hybridization of labeled CA nucleic acid probes to tissue arrays is done. For example, arrays of tissue samples, including CA tissue and/or normal tissue, are made. In situ hybridization as is known in the art can then be done.

It is understood that when comparing the expression fingerprints between an individual and a standard, the skilled artisan can make a diagnosis as well as a prognosis. It is further understood that the genes which indicate the diagnosis may differ from those which indicate the prognosis.

In a preferred embodiment, the CA proteins, antibodies, nucleic acids, modified proteins and cells containing CA sequences are used in prognosis assays. As above, gene expression profiles can be generated that correlate to carcinoma, especially lymphoma, severity, in terms of long term prognosis. Again, this may be done on either a protein or gene level, with the use of genes being preferred. As above, the CA probes are attached to biochips for the detection and quantification of CA sequences in a tissue or patient. The assays proceed as outlined for diagnosis.

In a preferred embodiment, any of the CA sequences as described herein are used in drug screening assays. The CA proteins, antibodies, nucleic acids, modified proteins and cells containing CA sequences are used in drug screening assays or by evaluating the effect of drug candidates on a “gene expression profile” or expression profile of polypeptides. In one embodiment, the expression profiles are used, preferably in conjunction with high throughput screening techniques to allow monitoring for expression profile genes after treatment with a candidate agent, Zlokarnik, et al., Science 279, 84-8 (1998), Heid, et al., Genome Res., 6:986-994 (1996).

In a preferred embodiment, the CA proteins, antibodies, nucleic acids, modified proteins and cells containing the native or modified CA proteins are used in screening assays. That is, the present invention provides novel methods for screening for compositions which modulate the carcinoma phenotype. As above, this can be done by screening for modulators of gene expression or for modulators of protein activity. Similarly, this may be done on an individual gene or protein level or by evaluating the effect of drug candidates on a “gene expression profile”. In a preferred embodiment, the expression profiles are used, preferably in conjunction with high throughput screening techniques to allow monitoring for expression profile genes after treatment with a candidate agent, see Zlokarnik, supra.

Having identified the CA genes herein, a variety of assays to evaluate the effects of agents on gene expression may be executed. In a preferred embodiment, assays may be run on an individual gene or protein level. That is, having identified a particular gene as aberrantly regulated in carcinoma, candidate bioactive agents may be screened to modulate the genes response. “Modulation” thus includes both an increase and a decrease in gene expression or activity. The preferred amount of modulation will depend on the original change of the gene expression in normal versus tumor tissue, with changes of at least 10%, preferably 50%, more preferably 100-300%, and in some embodiments 300-1000% or greater. Thus, if a gene exhibits a 4 fold increase in tumor compared to normal tissue, a decrease of about four fold is desired; a 10 fold decrease in tumor compared to normal tissue gives a 10 fold increase in expression for a candidate agent is desired, etc. Alternatively, where the CA sequence has been altered but shows the same expression profile or an altered expression profile, the protein will be detected as outlined herein.

As will be appreciated by those in the art, this may be done by evaluation at either the gene or the protein level; that is, the amount of gene expression may be monitored using nucleic acid probes and the quantification of gene expression levels, or, alternatively, the level of the gene product itself can be monitored, for example through the use of antibodies to the CA protein and standard immunoassays. Alternatively, binding and bioactivity assays with the protein may be done as outlined below.

In a preferred embodiment, gene expression monitoring is done and a number of genes, i.e. an expression profile, is monitored simultaneously, although multiple protein expression monitoring can be done as well.

In this embodiment, the CA nucleic acid probes are attached to biochips as outlined herein for the detection and quantification of CA sequences in a particular cell. The assays are further described below.

Generally, in a preferred embodiment, a candidate bioactive agent is added to the cells prior to analysis. Moreover, screens are provided to identify a candidate bioactive agent which modulates a particular type of carcinoma, modulates CA proteins, binds to a CA protein, or interferes between the binding of a CA protein and an antibody.

The term “candidate bioactive agent” or “drug candidate” or grammatical equivalents as used herein describes any molecule, e.g., protein, oligopeptide, small organic or inorganic molecule, polysaccharide, polynucleotide, etc., to be tested for bioactive agents that are capable of directly or indirectly altering either the carcinoma phenotype, binding to and/or modulating the bioactivity of an CA protein, or the expression of a CA sequence, including both nucleic acid sequences and protein sequences. In a particularly preferred embodiment, the candidate agent suppresses a CA phenotype, for example to a normal tissue fingerprint. Similarly, the candidate agent preferably suppresses a severe CA phenotype. Generally a plurality of assay mixtures are run in parallel with different agent concentrations to obtain a differential response to the various concentrations. Typically, one of these concentrations serves as a negative control, i.e., at zero concentration or below the level of detection.

In one aspect, a candidate agent will neutralize the effect of an CA protein. By “neutralize” is meant that activity of a protein is either inhibited or counter acted against so as to have substantially no effect on a cell.

Candidate agents encompass numerous chemical classes, though typically they are organic or inorganic molecules, preferably small organic compounds having a molecular weight of more than 100 and less than about 2,500 daltons. Preferred small molecules are less than 2000, or less than 1500 or less than 1000 or less than 500 D. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In a preferred embodiment, the candidate bioactive agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations.

In a preferred embodiment, the candidate bioactive agents are naturally occurring proteins or fragments of naturally occurring proteins. Thus, for example, cellular extracts containing proteins, or random or directed digests of proteinaceous cellular extracts, may be used. In this way libraries of procaryotic and eucaryotic proteins may be made for screening in the methods of the invention. Particularly preferred in this embodiment are libraries of bacterial, fungal, viral, and mammalian proteins, with the latter being preferred, and human proteins being especially preferred.

In a preferred embodiment, the candidate bioactive agents are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

In a preferred embodiment, the candidate bioactive agents are nucleic acids, as defined above.

As described above generally for proteins, nucleic acid candidate bioactive agents may be naturally occurring nucleic acids, random nucleic acids, or “biased” random nucleic acids. For example, digests of procaryotic or eucaryotic genomes may be used as is outlined above for proteins.

In a preferred embodiment, the candidate bioactive agents are organic chemical moieties, a wide variety of which are available in the literature.

In assays for altering the expression profile of one or more CA genes, after the candidate agent has been added and the cells allowed to incubate for some period of time, the sample containing the target sequences to be analyzed is added to the biochip. If required, the target sequence is prepared using known techniques. For example, the sample may be treated to lyse the cells, using known lysis buffers, electroporation, etc., with purification and/or amplification such as PCR occurring as needed, as will be appreciated by those in the art. For example, an in vitro transcription with labels covalently attached to the nucleosides is done. Generally, the nucleic acids are labeled with a label as defined herein, with biotin-FITC or PE, cy3 and cy5 being particularly preferred.

In a preferred embodiment, the target sequence is labeled with, for example, a fluorescent, chemiluminescent, chemical, or radioactive signal, to provide a means of detecting the target sequence's specific binding to a probe. The label also can be an enzyme, such as, alkaline phosphatase or horseradish peroxidase, which when provided with an appropriate substrate produces a product that can be detected. Alternatively, the label can be a labeled compound or small molecule, such as an enzyme inhibitor, that binds but is not catalyzed or altered by the enzyme. The label also can be a moiety or compound, such as, an epitope tag or biotin which specifically binds to streptavidin. For the example of biotin, the streptavidin is labeled as described above, thereby, providing a detectable signal for the bound target sequence. As known in the art, unbound labeled streptavidin is removed prior to analysis.

As will be appreciated by those in the art, these assays can be direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, and then added to the biochip comprising a plurality of nucleic acid probes, under conditions that allow the formation of a hybridization complex.

A variety of hybridization conditions may be used in the present invention, including high, moderate and low stringency conditions as outlined above. The assays are generally run under stringency conditions which allows formation of the label probe hybridization complex only in the presence of target. Stringency can be controlled by altering a step parameter that is a thermodynamic variable, including, but not limited to, temperature, formamide concentration, salt concentration, chaotropic salt concentration pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The reactions outlined herein may be accomplished in a variety of ways, as will be appreciated by those in the art. Components of the reaction may be added simultaneously, or sequentially, in any order, with preferred embodiments outlined below. In addition, the reaction may include a variety of X other reagents may be included in the assays. These include reagents like salts, buffers, neutral proteins, e.g. albumin, detergents, etc which may be used to facilitate optimal hybridization and detection, and/or reduce non-specific or background interactions. Also reagents that otherwise improve the efficiency of the assay, such as protease inhibitors, nuclease inhibitors, anti-microbial agents, etc., may be used, depending on the sample preparation methods and purity of the target. In addition, either solid phase or solution based (i.e., kinetic PCR) assays may be used.

Once the assay is run, the data is analyzed to determine the expression levels, and changes in expression levels as between states, of individual genes, forming a gene expression profile.

In a preferred embodiment, as for the diagnosis and prognosis applications, having identified the differentially expressed gene(s) or mutated gene(s) important in any one state, screens can be run to alter the expression of the genes individually. That is, screening for modulation of regulation of expression of a single gene can be done. Thus, for example, particularly in the case of target genes whose presence or absence is unique between two states, screening is done for modulators of the target gene expression.

In addition, screens can be done for novel genes that are induced in response to a candidate agent. After identifying a candidate agent based upon its ability to suppress a CA expression pattern leading to a normal expression pattern, or modulate a single CA gene expression profile so as to mimic the expression of the gene from normal tissue, a screen as described above can be performed to identify genes that are specifically modulated in response to the agent. Comparing expression profiles between normal tissue and agent treated CA tissue reveals genes that are not expressed in normal tissue or CA tissue, but are expressed in agent treated tissue. These agent specific sequences can be identified and used by any of the methods described herein for CA genes or proteins. In particular these sequences and the proteins they encode find use in marking or identifying agent treated cells. In addition, antibodies can be raised against the agent induced proteins and used to target novel therapeutics to the treated CA tissue sample.

Thus, in one embodiment, a candidate agent is administered to a population of CA cells, that thus has an associated CA expression profile. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e. a peptide) may be put into a viral construct such as a retroviral construct and added to the cell, such that expression of the peptide agent is accomplished; see PCT US97/01019, hereby expressly incorporated by reference.

Once the candidate agent has been administered to the cells, the cells can be washed if desired and are allowed to incubate under preferably physiological conditions for some period of time. The cells are then harvested and a new gene expression profile is generated, as outlined herein.

Thus, for example, CA tissue may be screened for agents that reduce or suppress the CA phenotype. A change in at least one gene of the expression profile indicates that the agent has an effect on CA activity. By defining such a signature for the CA phenotype, screens for new drugs that alter the phenotype can be devised. With this approach, the drug target need not be known and need not be represented in the original expression screening platform, nor does the level of transcript for the target protein need to change.

In a preferred embodiment, as outlined above, screens may be done on individual genes and gene products (proteins). That is, having identified a particular differentially expressed gene as important in a particular state, screening of modulators of either the expression of the gene or the gene product itself can be done. The gene products of differentially expressed genes are sometimes referred to herein as “CA proteins” or an “CAP”. The CAP may be a fragment, or alternatively, be the full length protein to the fragment encoded by the nucleic acids of Tables 1-10. Preferably, the CAP is a fragment. In another embodiment, the sequences are sequence variants as further described herein.

Preferably, the CAP is a fragment of approximately 14 to 24 amino acids long. More preferably the fragment is a soluble fragment. Preferably, the fragment includes a non-transmembrane region. In a preferred embodiment, the fragment has an N-terminal Cys to aid in solubility. In one embodiment, the c-terminus of the fragment is kept as a free acid and the n-terminus is a free amine to aid in coupling, i.e., to cysteine.

In one embodiment the CA proteins are conjugated to an immunogenic agent as discussed herein. In one embodiment the CA protein is conjugated to BSA.

In a preferred embodiment, screening is done to alter the biological function of the expression product of the CA gene. Again, having identified the importance of a gene in a particular state, screening for agents that bind and/or modulate the biological activity of the gene product can be run as is more fully outlined below.

In a preferred embodiment, screens are designed to first find candidate agents that can bind to CA proteins, and then these agents may be used in assays that evaluate the ability of the candidate agent to modulate the CAP activity and the carcinoma phenotype. Thus, as will be appreciated by those in the art, there are a number of different assays which may be run; binding assays and activity assays.

In a preferred embodiment, binding assays are done. In general, purified or isolated gene product is used; that is, the gene products of one or more CA nucleic acids are made. In general, this is done as is known in the art. For example, antibodies are generated to the protein gene products, and standard immunoassays are run to determine the amount of protein present. Alternatively, cells comprising the CA proteins can be used in the assays.

Thus, in a preferred embodiment, the methods comprise combining a CA protein and a candidate bioactive agent, and determining the binding of the candidate agent to the CA protein. Preferred embodiments utilize the human or mouse CA protein, although other mammalian proteins may also be used, for example for the development of animal models of human disease. In some embodiments, as outlined herein, variant or derivative CA proteins may be used.

Generally, in a preferred embodiment of the methods herein, the CA protein or the candidate agent is non-diffusably bound to an insoluble support having isolated sample receiving areas (e.g. a microtiter plate, an array, etc.). The insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon™, etc. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

In a preferred embodiment, the CA protein is bound to the support, and a candidate bioactive agent is added to the assay. Alternatively, the candidate agent is bound to the support and the CA protein is added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the candidate bioactive agent to the CA protein may be done in a number of ways. In a preferred embodiment, the candidate bioactive agent is labeled, and binding determined directly. For example, this may be done by attaching all or a portion of the CA protein to a solid support, adding a labeled candidate agent (for example a fluorescent label), washing off excess reagent, and determining whether the label is present on the solid support. Various blocking and washing steps may be utilized as is known in the art.

By “labeled” herein is meant that the compound is either directly or indirectly labeled with a label which provides a detectable signal, e.g. radioisotope, fluorescers, enzyme, antibodies, particles such as magnetic particles, chemiluminescers, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

In some embodiments, only one of the components is labeled. For example, the proteins (or proteinaceous candidate agents) may be labeled at tyrosine positions using ¹²⁵I , or with fluorophores. Alternatively, more than one component may be labeled with different labels; using ¹²⁵I for the proteins, for example, and a fluorophor for the candidate agents.

In a preferred embodiment, the binding of the candidate bioactive agent is determined through the use of competitive binding assays. In this embodiment, the competitor is a binding moiety known to bind to the target molecule (i.e. CA protein), such as an antibody, peptide, binding partner, ligand, etc. Under certain circumstances, there may be competitive binding as between the bioactive agent and the binding moiety, with the binding moiety displacing the bioactive agent.

In one embodiment, the candidate bioactive agent is labeled. Either the candidate bioactive agent, or the competitor, or both, is added first to the protein for a time sufficient to allow binding, if present. Incubations may be performed at any temperature which facilitates optimal activity, typically between 4 and 40° C. Incubation periods are selected for optimum activity, but may also be optimized to facilitate rapid high through put screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the candidate bioactive agent. Displacement of the competitor is an indication that the candidate bioactive agent is binding to the CA protein and thus is capable of binding to, and potentially modulating, the activity of the CA protein. In this embodiment, either component can be labeled. Thus, for example, if the competitor is labeled, the presence of label in the wash solution indicates displacement by the agent. Alternatively, if the candidate bioactive agent is labeled, the presence of the label on the support indicates displacement.

In an alternative embodiment, the candidate bioactive agent is added first, with incubation and washing, followed by the competitor. The absence of binding by the competitor may indicate that the bioactive agent is bound to the CA protein with a higher affinity. Thus, if the candidate bioactive agent is labeled, the presence of the label on the support, coupled with a lack of competitor binding, may indicate that the candidate agent is capable of binding to the CA protein.

In a preferred embodiment, the methods comprise differential screening to identity bioactive agents that are capable of modulating the activity of the CA proteins. In this embodiment, the methods comprise combining a CA protein and a competitor in a first sample. A second sample comprises a candidate bioactive agent, a CA protein and a competitor. The binding of the competitor is determined for both samples, and a change, or difference in binding between the two samples indicates the presence of an agent capable of binding to the CA protein and potentially modulating its activity. That is, if the binding of the competitor is different in the second sample relative to the first sample, the agent is capable of binding to the CA protein.

Alternatively, a preferred embodiment utilizes differential screening to identify drug candidates that bind to the native CA protein, but cannot bind to modified CA proteins. The structure of the CA protein may be modeled, and used in rational drug design to synthesize agents that interact with that site. Drug candidates that affect CA bioactivity are also identified by screening drugs for the ability to either enhance or reduce the activity of the protein.

Positive controls and negative controls may be used in the assays. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g. albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

Screening for agents that modulate the activity of CA proteins may also be done. In a preferred embodiment, methods for screening for a bioactive agent capable of modulating the activity of CA proteins comprise the steps of adding a candidate bioactive agent to a sample of CA proteins, as above, and determining an alteration in the biological activity of CA proteins. “Modulating the activity of an CA protein” includes an increase in activity, a decrease in activity, or a change in the type or kind of activity present. Thus, in this embodiment, the candidate agent should both bind to CA proteins (although this may not be necessary), and alter its biological or biochemical activity as defined herein. The methods include both in vitro screening methods, as are generally outlined above, and in vivo screening of cells for alterations in the presence, distribution, activity or amount of CA proteins.

Thus, in this embodiment, the methods comprise combining a CA sample and a candidate bioactive agent, and evaluating the effect on CA activity. By “CA activity” or grammatical equivalents herein is meant one of the CA protein's biological activities, including, but not limited to, its role in tumorigenesis, including cell division, preferably in lymphatic tissue, cell proliferation, tumor growth and transformation of cells. In one embodiment, CA activity includes activation of or by a protein encoded by a nucleic acid of Tables 1-10. An inhibitor of CA activity is the inhibition of any one or more CA activities.

In a preferred embodiment, the activity of the CA protein is increased; in another preferred embodiment, the activity of the CA protein is decreased. Thus, bioactive agents that are antagonists are preferred in some embodiments, and bioactive agents that are agonists may be preferred in other embodiments.

In a preferred embodiment, the invention provides methods for screening for bioactive agents capable of modulating the activity of a CA protein. The methods comprise adding a candidate bioactive agent, as defined above, to a cell comprising CA proteins. Preferred cell types include almost any cell. The cells contain a recombinant nucleic acid that encodes a CA protein. In a preferred embodiment, a library of candidate agents are tested on a plurality of cells.

In one aspect, the assays are evaluated in the presence or absence or previous or subsequent exposure of physiological signals, for example hormones, antibodies, peptides, antigens, cytokines, growth factors, action potentials, pharmacological agents including chemotherapeutics, radiation, carcinogenics, or other cells (i.e. cell-cell contacts). In another example, the determinations are determined at different stages of the cell cycle process.

In this way, bioactive agents are identified. Compounds with pharmacological activity are able to enhance or interfere with the activity of the CA protein.

In one embodiment, a method of inhibiting carcinoma cancer cell division, is provided. The method comprises administration of a carcinoma cancer inhibitor.

In a preferred embodiment, a method of inhibiting lymphoma carcinoma cell division is provided comprising administration of a lymphoma carcinoma inhibitor.

In another embodiment, a method of inhibiting tumor growth is provided. The method comprises administration of a carcinoma cancer inhibitor. In a particularly preferred embodiment, a method of inhibiting tumor growth in lymphatic tissue is provided comprising administration of a lymphoma inhibitor.

In a further embodiment, methods of treating cells or individuals with cancer are provided. The method comprises administration of a carcinoma cancer inhibitor. Preferably, the carcinoma is a lymphoma carcinoma.

In one embodiment, a carcinoma cancer inhibitor is an antibody as discussed above. In another embodiment, the carcinoma cancer inhibitor is an antisense molecule. Antisense molecules as used herein include antisense or sense oligonucleotides comprising a singe-stranded nucleic acid sequence (either RNA or DNA) capable of binding to target mRNA (sense) or DNA (antisense) sequences for carcinoma cancer molecules. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, for example, Stein and Cohen, Cancer Res. 48:2659, (1988) and van der Krol et al., BioTechniques 6:958, (1988).

Antisense molecules may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a sense or an antisense oligonucleotide may be introduced into a cell containing the target nucleic acid sequence by formation of an oligonucleotide-lipid complex, as described in WO 90/10448. It is understood that the use of antisense molecules or knock out and knock in models may also be used in screening assays as discussed above, in addition to methods of treatment.

The compounds having the desired pharmacological activity may be administered in a physiologically acceptable carrier to a host, as previously described. The agents may be administered in a variety of ways, orally, parenterally e.g., subcutaneously, intraperitoneally, intravascularly, etc. Depending upon the manner of introduction, the compounds may be formulated in a variety of ways. The concentration of therapeutically active compound in the formulation may vary from about 0.1-100% wgt/vol. The agents may be administered alone or in combination with other treatments, i.e., radiation.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

Without being bound by theory, it appears that the various CA sequences are important in carcinomas. Accordingly, disorders based on mutant or variant CA genes may be determined. In one embodiment, the invention provides methods for identifying cells containing variant CA genes comprising determining all or part of the sequence of at least one endogenous CA genes in a cell. As will be appreciated by those in the art, this may be done using any number of sequencing techniques. In a preferred embodiment, the invention provides methods of identifying the CA genotype of an individual comprising determining all or part of the sequence of at least one CA gene of the individual. This is generally done in at least one tissue of the individual, and may include the evaluation of a number of tissues or different samples of the same tissue. The method may include comparing the sequence of the sequenced CA gene to a known CA gene, i.e., a wild-type gene. As will be appreciated by those in the art, alterations in the sequence of some oncogenes can be an indication of either the presence of the disease, or propensity to develop the disease, or prognosis evaluations.

The sequence of all or part of the CA gene can then be compared to the sequence of a known CA gene to determine if any differences exist. This can be done using any number of known homology programs, such as Bestfit, etc. In a preferred embodiment, the presence of a difference in the sequence between the CA gene of the patient and the known CA gene is indicative of a disease state or a propensity for a disease state, as outlined herein.

In a preferred embodiment, the CA genes are used as probes to determine the number of copies of the CA gene in the genome. For example, some cancers exhibit chromosomal deletions or insertions, resulting in an alteration in the copy number of a gene.

In another preferred embodiment CA genes are used as probes to determine the chromosomal location of the CA genes. Information such as chromosomal location finds use in providing a diagnosis or prognosis in particular when chromosomal abnormalities such as translocations, and the like are identified in CA gene loci.

Thus, in one embodiment, methods of modulating CA in cells or organisms are provided. In one embodiment, the methods comprise administering to a cell an anti-CA antibody that reduces or eliminates the biological activity of an endogenous CA protein. Alternatively, the methods comprise administering to a cell or organism a recombinant nucleic acid encoding a CA protein. As will be appreciated by those in the art, this may be accomplished in any number of ways. In a preferred a embodiment, for example when the CA sequence is down-regulated in carcinoma, the activity of the CA gene is increased by increasing the amount of CA in the cell, for example by overexpressing the endogenous CA or by administering a gene encoding the CA sequence, using known gene-therapy techniques, for example. In a preferred embodiment, the gene therapy techniques include the incorporation of the exogenous gene using enhanced homologous recombination (EHR), for example as described in PCT/US93/03868, hereby incorporated by reference in its entirety. Alternatively, for example when the CA sequence is up-regulated in carcinoma, the activity of the endogenous CA gene is decreased, for example by the administration of a CA antisense nucleic acid.

In one embodiment, the CA proteins of the present invention may be used to generate polyclonal and monoclonal antibodies to CA proteins, which are useful as described herein. Similarly, the CA proteins can be coupled, using standard technology, to affinity chromatography columns. These columns may then be used to purify CA antibodies. In a preferred embodiment, the antibodies are generated to epitopes unique to a CA protein; that is, the antibodies show little or no cross-reactivity to other proteins. These antibodies find use in a number of applications. For example, the CA antibodies may be coupled to standard affinity chromatography columns and used to purify CA proteins. The antibodies may also be used as blocking polypeptides, as outlined above, since they will specifically bind to the CA protein.

In one embodiment, a therapeutically effective dose of a CA or modulator thereof is administered to a patient. By “therapeutically effective dose” herein is meant a dose that produces the effects for which it is administered. The exact dose will depend on the purpose of the treatment, and will be ascertainable by one skilled in the art using known techniques. As is known in the art, adjustments for CA degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

A “patient” for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human.

The administration of the CA proteins and modulators of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the CA proteins and modulators may be directly applied as a solution or spray.

The pharmaceutical compositions of the present invention comprise a CA protein in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol. Additives are well known in the art, and are used in a variety of formulations.

In a preferred embodiment, CA proteins and modulators are administered as therapeutic agents, and can be formulated as outlined above. Similarly, CA genes (including both the full-length sequence, partial sequences, or regulatory sequences of the CA coding regions) can be administered in gene therapy applications, as is known in the art. These CA genes can include antisense applications, either as gene therapy (i.e. for incorporation into the genome) or as antisense compositions, as will be appreciated by those in the art.

In a preferred embodiment, CA genes are administered as DNA vaccines, either single genes or combinations of CA genes. Naked DNA vaccines are generally known in the art. Brower, Nature Biotechnology, 16:1304-1305 (1998).

In one embodiment, CA genes of the present invention are used as DNA vaccines. Methods for the use of genes as DNA vaccines are well known to one of ordinary skill in the art, and include placing a CA gene or portion of a CA gene under the control of a promoter for expression in a patient with carcinoma. The CA gene used for DNA vaccines can encode full-length CA proteins, but more preferably encodes portions of the CA proteins including peptides derived from the CA protein. In a preferred embodiment a patient is immunized with a DNA vaccine comprising a plurality of nucleotide sequences derived from a CA gene. Similarly, it is possible to immunize a patient with a plurality of CA genes or portions thereof as defined herein. Without being bound by theory, expression of the polypeptide encoded by the DNA vaccine, cytotoxic T-cells, helper T-cells and antibodies are induced which recognize and destroy or eliminate cells expressing CA proteins.

In a preferred embodiment, the DNA vaccines include a gene encoding an adjuvant molecule with the DNA vaccine. Such adjuvant molecules include cytokines that increase the immunogenic response to the CA polypeptide encoded by the DNA vaccine. Additional or alternative adjuvants are known to those of ordinary skill in the art and find use in the invention.

In another preferred embodiment CA genes find use in generating animal models of carcinomas, particularly lymphoma carcinomas. As is appreciated by one of ordinary skill in the art, when the CA gene identified is repressed or diminished in CA tissue, gene therapy technology wherein antisense RNA directed to the CA gene will also diminish or repress expression of the gene. An animal generated as such serves as an animal model of CA that finds use in screening bioactive drug candidates. Similarly, gene knockout technology, for example as a result of homologous recombination with an appropriate gene targeting vector, will result in the absence of the CA protein. When desired, tissue-specific expression or knockout of the CA protein may be necessary.

It is also possible that the CA protein is overexpressed in carcinoma. As such, transgenic animals can be generated that overexpress the CA protein. Depending on the desired expression level, promoters of various strengths can be employed to express the transgene. Also, the number of copies of the integrated transgene can be determined and compared for a determination of the expression level of the transgene. Animals generated by such methods find use as animal models of CA and are additionally useful in screening for bioactive molecules to treat carcinoma.

The CA nucleic acid sequences of the invention are depicted in Tables 1-10. The sequences in each Table include genomic sequence, mRNA and coding sequences for both mouse and human. The different sequences are assigned the following SEQ ID Nos:

Table I (mouse gene: Fscn1; human gene SNL)

Mouse genomic sequence (SEQ ID NO: 1)

Mouse mRNA sequence (SEQ ID NO: 2)

Mouse coding sequence (SEQ ID NO: 3)

Human genomic sequence (SEQ ID NO: 4)

Human mRNA sequence (SEQ ID NO: 5)

Human coding sequence (SEQ ID NO: 6)

Table II (mouse gene Map3k6; human gene MAP3K6)

Mouse genomic sequence (SEQ ID NO: 7)

Mouse mRNA sequence (SEQ ID NO: 8)

Mouse coding sequence (SEQ ID NO: 9)

Human genomic sequence (SEQ ID NO: 10)

Human mRNA sequence (SEQ ID NO: 11)

Human coding sequence (SEQ ID NO: 12)

Table III (mouse gene Fosb; human gene FOSB)

Mouse genomic sequence (SEQ ID NO: 13)

Mouse mRNA sequence (SEQ ID NO: 14)

Mouse coding sequence (SEQ ID NO: 15)

Human genomic sequence (SEQ ID NO: 16)

Human mRNA sequence (SEQ ID NO: 17)

Human coding sequence (SEQ ID NO: 18)

Table IV (mouse gene cmkbr7; human gene: CCR7)

Mouse genomic sequence (SEQ ID NO: 19)

Mouse mRNA sequence (SEQ ID NO: 20)

Mouse coding sequence (SEQ ID NO: 21)

Human genomic sequence (SEQ ID NO: 22)

Human mRNA sequence (SEQ ID NO: 23)

Human coding sequence (SEQ ID NO: 24)

Table V (mouse gene: Ccnd1; human gene: CCND1)

Mouse genomic sequence (SEQ ID NO: 25)

Mouse mRNA sequence (SEQ ID NO: 26)

Mouse coding sequence (SEQ ID NO: 27)

Human genomic sequence (SEQ ID NO: 28)

Human mRNA sequence (SEQ ID NO:29)

Human coding sequence (SEQ ID NO: 30)

Table VI (mouse gene: Ccnd3; human gene: CCND3)

Mouse genomic sequence (SEQ ID NO: 31)

Mouse mRNA sequence (SEQ ID NO: 32)

Mouse coding sequence (SEQ ID NO: 33)

Human genomic sequence (SEQ ID NO: 34)

Human mRNA sequence (SEQ ID NO: 35)

Human coding sequence (SEQ ID NO: 36)

Table VII (mouse gene: Wnt3; human gene: WNT3)

Mouse genomic sequence (SEQ ID NO: 37)

Mouse mRNA sequence (SEQ ID NO: 38)

Mouse coding sequence (SEQ ID NO: 39)

Human genomic sequence (SEQ ID NO: 40)

Human mRNA sequence (SEQ ID NO: 41)

Human coding sequence (SEQ ID NO: 42)

Table VII (mouse gene: Batf; human gene: BATF)

Mouse genomic sequence (SEQ ID NO: 43)

Mouse mRNA sequence (SEQ ID NO: 44)

Mouse coding sequence (SEQ ID NO: 45)

Human genomic sequence (SEQ ID NO: 46)

Human mRNA sequence (SEQ ID NO: 47)

Human coding sequence (SEQ ID NO: 48)

Table IX (mouse gene: Irf4; human gene: IRF4)

Mouse genomic sequence (SEQ ID NO: 49)

Mouse mRNA sequence (SEQ ID NO: 50)

Mouse coding sequence (SEQ ID NO: 51)

Human genomic sequence (SEQ ID NO: 52)

Human mRNA sequence (SEQ ID NO: 53)

Human coding sequence (SEQ ID NO: 54)

Table X (mouse gene: Notch1; human gene: NOTCH1)

Mouse genomic sequence (SEQ ID NO: 55)

Mouse mRNA sequence (SEQ ID NO: 56)

Mouse coding sequence (SEQ ID NO: 57)

Human genomic sequence (SEQ ID NO: 58)

Human mRNA sequence (SEQ ID NO: 59)

Human coding sequence (SEQ ID NO: 60)

TABLE I

MOUSE NOMENCLATURE
ICSGNM Fscn1
Celera mCG23208
HUMAN NOMENCLATURE
HGNC SNL
Celera hCG15970
MOUSE SEQUENCE - GENOMIC
CCTCTCAAACTCTTGGTAATTCTCCTGCTTCGGCACGCTGGGGACTGGGACTACACCTGTGTGCCACCATGGCTGGCTATTTCTCTCTCTTGAACAC
TGGAAGAGTGCTCAGAGTTTACTGGATCTGGGAGAAGCTCGAGGCTGGGTTATGGAAGCCGGCTCTGCTTGCTCCAGCTCAGGGAGGAGTTGCCTGA
GGGCCAGGCACTTCGAAGGACAGATGGGACCCAAGGCCAGACACTGGGCCCCAGCTCACCAGAAGTGGAGCCCTGACTGTCTCTGAGAGGGTAAGCT
GGGGTGGCTGCCAGGCGGGCAAGGCCAAAGCCTGGCAGCAGCCGGTGGCCCCTCTTCTGGCAGAGATATCTTGGCATGAGCCTGGCTCTGCCCATGA
CACTAAAGTGCCCTCTTAATTAGCCAGGCTCTTGCCAAGCACTGGCCACGATTGCCTTGTTTCACAGAATTCACCTTGGACTGGCACAGATGGGGAG
GGTGGATAGCCCGGTGTTTTGTCTTTCTTCTAGGGGAGCTGGGCTCAAGGGCAGGACTCCTGGGCCAGCCCAGGTTTTTCCCTCAGAAACCACAGCA
TGAATACTGGCATGATGGAGCACACCTGTAATCAGCAGAAATTGAGACAGGAAGATTGTTGAGAATTTGAGGCCAACCTAGGCTAAATAGGGAGACC
CTATCTCTACACACTCCCCCTCCCCCCGCCCCTGGAAATGCTGGAGGGTCTGGGGAAGATGGCTCAGTGGTTAAGGGCACTTGCTGCTCTTGCAGGG
GACCCAAGTTTGATTCCCAGCATCTATAACTTGGTGGCTCACAATTCCAGTTCCAGGCACTCTGACACCTTCCTGTGGTCTGCAAGGCACCTGCATT
CATTCTCACGTGCATGCGCACACACATGCGCGCGCGCACACACACACACACACACACACACACATCTCATAAATACAAATAAAATAAATCTTGAAAA
CAACAGTGACAACAACACATGCTGAACATGGTGGTGCTCCTGACATGGTGGTGCTCCTGACATGGTGGTGCTCCTGACATGGTGATGCTCCTGACAT
GGTGGTGCTCCTGACATGATGGTAAGCCTGACATGGTGGTGCTCCTGACATGGTGGTGCTCCTGACATGGTGATGCTCCTGACATGGTGGTGCTCCT
GACATGGTGGTAAGCCTGACATGGTGGTAAGCCTGACATGGTGGTGCTCCTGACATGGTGGTGCTCCCTGCACTGACAGTTCTGAGGAGGTGGAGAT
AGGAGGATCCAGACTTGGAAGCTTGCCTGAGATAGACCCTGTCTCAGAATAGGCTGGAGGCTGCCTCTGCCCTTTTGCTTCTATCTCTTTTTCCATA
TGTGTGTGTGCGTGTGTGCATATGTGTATGCATATTTGTAAGTATATGTATATAAATAAACACACACACACACAAACTTACTCTGTATACTAGGCTG
GCCTTGAACTCAGAGACCTGCCTGTTTCTGCCTTCCCAGTGCTGGGATTAAAAGCGTGTGCCATGGGCTGGTGAGATGGCTCAGTGGGTAAGAGTAC
CTGACTGCTCTTCCAAAGGTCCAGTGTTCAAGTCCCAGCAACCACATGGTGGCTCACAACCATCCGTAACAAGATCTGACGCCCTCTTCTGGTGTGT
CTGAAGACAGCTACAGTGTACTTAAATAAATAAATCTTAAAAAAAAAAAAAAAAAAAAAAAAAGGCGTGCACCACCACTGCCCGGCTAGTTAGCTTT
ATCTTCCGTGGATGTTTTGTCTGCAGTATGTCTGAGTGAGGGTGTCAGATCCTCTGTAACTGGAGTTATAGACAGTTGTGAGCAGCCATGTAAGTGC
TGGGAATTGAATCCGGGTTCTCTGGAAGAGCAGTTCATGCTCTTAAGCACTGCGCCATCTCTCCAGCTGTCCTTATTTATTTTTAAGGGTCTCTTGT
AGCCATACAGGCCAGAAACCCACCATTAGCCACGGACCTTGAGCTCCAAGTGCTGGGTGACAGGCCCGCATCTCTCCTGGTTTGTGATCTGCTAGGG
ATGGGGTCTAGGACCTCACACAGGCTGCATAAGCATTCCCCTCACCCACCCTCATCCCCACCCAGTCCTGGACAGGCTCTGGCTGTGTAGGCTGGTC
TCCTCCTTTCTATCCCCCTGCCTCAGCCTCCTGAGTGCTGAGGNNNNNNNNNNNNNNNNNNNNNNNNNNNNNNNNNNNNNNNNNNNNNNNNNNNNNN
NNNNNNNNNNNNNNNNNNNNNNNNNNNNNNNCTGTGGTGGCTCCCGAGTGCTGTCATCCCAGCACTGGGGAGGTTGAAGCAGGAAGGAGAATCACAG
GTTCAAGGCCAGCTTGGTTTACTTGAGACTCTGTCTCAAAAAGACCAAACCAGGGACTGGGAGGATGGCCAGTAAAATGCTTTTCCAGCTATCGAGG
GAACCTGAGTTTCATCCTGGAACTCATACAAAAAGCTGGCTGTGGTCAGGTGGCGCATGCCTTTAATCCCATCACTCAGGAGACAGAGGCAGGCAGA
TTCTTGAGTTCGAGGCCAGCCTGGTCTACAGACTGAGTTCCAGGATAGCCAGGTCTACACAGAGAAACCCTATCTTGGAAAACAAAACAAACAAAAA
AAACTAGGTGTGGCTAGCCTGTAACCCCAGTGCTGGGTAAACAGGGGCAGGTGAATACATCTCCGGGATTCGCCAGCCTGTCCTATCTATGAGCTCT
AGGTCTGGGGAGACAGCCTGTCAAATCAATGCAAGCAGGTGACAACCGAGGAAAGACACCCAAGGTTGATGCACGGGCAAGTGTGCACACACACACA
CCACAGCAAACAAAAACTAACAAAACCCAAACCATATTTTGCTGTGGTGGTGGTTGTTAAATTTTATTTATTCTGTGTGTATGGGTATTTCACCTGC
ATGAATATCTACCTGTGTAGCATATACATGCCGGGTACTTTCGGAGGTCAGAAGAGGACATCAAATCCCGTAGAACTGGAGTTACAGAAAATGAACT
TGTGGCTTCTGGGAATCTAACTTGGATCCTCTAAAGGACAGGCAGGACTCTGAATTCTTGAGCCACAGATACAGCTTTCCAAACCCATGTTTTGTAA
GAGCCACCAGGCCTGAAAACTCTGGGCCATGTCCCGTTTGTTGTGTCTAGGCCTCAGTTTCCTGGAAGGCAGAGAATGCGTAGGTCCGTTGTTAGTG
GAGACAGCAGTGGTGGTAACAGCCTGGTCTACCCTCTTGGTCAGAGGGAGGCAGGCGGAGAGTAACTCAGGGCCTGGGGGCAACACCCAGCTGGGCC
TGGGGCTAGTGGGGGCTCAGCCAGGCAGGACGTGGGTCCTCCTACTTCCTAAGCTCTGATGGGCAAGTGGTGCCAGTGGTACCAGGCTGCCCTGAGG
GGCCCTTAGAGCAGGTGACCACAGAGCCACAAAGAGGCTATTCATGGCCTCCGGTTCACGAGGCTGCCCCTTTATTGAGGGCTTGAACCACAGAAAG
CTGTGTGGTCTGCAGGGAAGCTAGTTCTGAGCTGTCTGGCCAGCTACAGTAGTGTGTGTGTGTGTGTGTGTGTGTATACACATGGTACTTTTTTCTA
TCTATCTATCTATCTATCTATCTATCTATCTATCTATCTATCTATCTATCTTTGTGTCTATGTATCTAATAATAAATTGATCTATTTAACTATGTAG
ATATCAATCCATTGATCTATGTATCTATCAACTGATGTGTATGTATGTATGGATGTACCTACATGCGTACTTGAGGCAGGACTTAAAGTAGCCCAGG
CTAGCCTTACCACATTTCATAGCAGAGGATGGTCTTGAACTTCAGGTCCTCCTGTCTCTACCTCGTGAGTGCTGGGATTACAGGTTTGTGCCACCAC
GCCTGGCTTAGGAGGTTCTGTGGTTAGAATCCAGGATTTCACTATCAACTGGGCCACATCGTCAGCCATATTCAATATGCTTTCTTTTACATTCTTA
CTTATTTACTACAAATGGGTCCCAGGAACCAAGGTCGGCAAGCCTGATGGTGAGTGCTCTTCCCTGCTGGCTGACTTATTTGCTCTTTTTTTTTTTT
TTTTTTTTTTCTAGACAGGGTTTCTCTGTGTAGCCCTGGCTGTCCTGGAACTCACTTTGTAGACCAGGCTGGCCTCGAACTCAGAAATCTGCCTGCC
TCTGTCCCCAACCCCACCCCCAGTACTGGGATTAAAGGCATGCGCCACACGCCCAGCTCTTATTTGCTCCTTTGAATTAAGGTCTCATGTACACAAG
GCTGACCGTTCATGCACAATACAGCTAAGGCTTGCTTTGAATTTCTCAGTTTCTTTCTTCTACCCAACAGTTTCTGAGATTTCAGGTCCGTCCTATG
TCATGCCCAGGTTTTTTGTTTGTTTGTTTGTTTGTTTTTTGGTTTTGTTTTGCTGTTTTTCAACACAGAGTTTCTCTGTGGAACCCTGGCTATCCTG
GAACTCTGTATACCAGGCTGTCCTTGAACTCAGAGACCCTCTGCCTCCCTAGTGCTGGGTTAAAGGTCCCTGCCATCACCGTGAAACTTAAGCCCAG
CTTCTCATTTGGTGGCTGGAAATTTGCCTGCCTCTGCCTCTCTCCAGCTGCCTAGCACTGATGTCTGGTTGAGAGCAGGGGAAACTGAGGTGCCGCT
AGGACAAGATCTCATGCAACCGTGAACAGCCCTACTGAGCCATCCTCCTGGGACAGCCTCTCAGGCCAGGCCTTCTGTGTCTTCTGCTATAGTAGCA
GTACAAAGTTGACACAACCTTGCTCTTCCCAGCAGCTGGGAGTTCCTGAGAAGTGTAAATGAGGTTTCCAGGAAAGCCAAGAGGTGAGAGACTGAAA
AGTTGGAGGGATAAGGGGGTGCCCATCAAGCTGCAGTGGAGACTTGTTATCTGTTGCGCATCCCTGACATCTCAGCAGGAGGATCAGGAGTTCAAGG
GCATCCTCAGCAACATAGTAAGTCTATGGAGCTAGCCTGGGCTACACAGATGCCATTACAAAAAAAAAAAAAAAAGTAAAAAAAAAATCTTGGGATA
TGGGGGATGGGTGGGTGAACTGTGTAGCCCAGGGAGTCACTACTGGTTCCTTTGGAAGTCCCTGTTCTGCATGGAGATCATGGAGATTCTGGGGACA
CTCCAGGAGTCCTCCTTCAGGGAACTAAGTTGGGGAGCAGGGCAGCCTATCCTGTGGCCTTATTTCCTGTCTGGTGCAAGCGCCAGGCAAGGCATCC
AGCCAGGAGCGACTCAGGCCTGACCTCCGCTAACTGTTCTGTTTTGTGCTGCACCCTCATCTCAGCAGGGATCAGGTGGCACTTGGGTCCCCAGGGG
ATCCAGGGACACAGTGTCCATGTCCCCATCTTTCCCCACAGGGACCTGGGGCAGGGCCGAAGAGGAGACCTCGTGATGTGTACAGGTGTAGCTCATC
CCTCCGAGGACTCAGGGGACACTTGGGGAAGGTGTGACACAGAGCAGGCTGGAGCTCTGTGCGGCTCTTTATTAAGTGTGCCTACAACAGGGGCAGA
GACACAGCAGCGACAATGATAACAGCCCACTCCCAACCGAAGCTCAATGGTTACCAGACTCAGCCAACTGCAGAATTCCAGTCCATAACGGCACCTC
CTTTCCTTTCTCAGGAGCTTACCAGAGAGGTCTCACCCCAAGCCTTCACCCCATCTAGAGCAAATGCTTATATCCCCCATGTTTGCTCAGAGCAGAG
CATGGAGGGACCTTCCTGTCACCAGAGGCAGAGGCATTCTGTATACGTGCATACACGCCTCTGTGTGTGTGTGTGTGTGTGTGTGTGTGTATTTGGT
ACAGATCTAAGCATAGAAAATTGAAAAGCATTGAGGTTGGTGATTGAAAGGCTCACTGATGGGCAAGTCGGGTTCGAGTGCGCTAACTGTATGACTC
TATGGATCCGAATATCATGCTGTTTTGGAAAGGAATGAGGGAGCTACTGTTTCTAAGGAAGAGAAGAATGGGCACCAAGACATAGGGAGGGAAGGTG
GGACAGTAAGCACTTACAGCCGAAGCCCAAGACCTGGAACCCAAGGGGAAAAACAGGTGTGGCCAGGCTTGGTGGCACGCGCCTTTAGTCTCTGAGT
TCAAGTCCAGCCAGGATTACATAGCAAAACTTTCCCTCAAGAAAAAAGGTAGGGGCTGGAGAGATGGCTCAGCAGTTAAGAGCTCTTCCGAAGGTCC
TGAGTTCAAATCCCAGCAACCACATGGTGGCTCACAACCATCTGTAATGAGATCTGACGCCCTCTTCTGGTGTGTCTGGAGACAGCTACAGTGTACT
TACATATAATAAATAAATAAATCCTAAAAAAAAAAAAAAAAGAAAAAGAAAAAGAAAAAAGGTAGAGGAGGAGGAAGAGGAGGAGGAGACGGCAAGA
CAAAACTAACTACATAGCTGGGGGAGGTGGTGCTTGCCTATAATCCCAGCACTTGGAAGGCTGAAAGAGGAAATCAGGAGTTCCTATGCAGCTTTGG
CTTATGTGAGCCTTTCTCATAAAACCAAGAGCAAAAAAGCAAAACATGAGGTGGTAACTCAGCTGGAGAGTGCTTGTCTAGATTCCCTCAGTGAGGG
GCTGGGGTGTGACTCAGTGCCTAGAATCCCCCAGGGAGGGGCTGGGGGCGTGGCTCAGTGGTAGAGCCCCTGCCTAGAATCCCCCATTGACGGGCTG
GGGTGTGGCTCAGTAACAGACTGCTCACCTAAGATGTTCAAGGCCATGACTTTCATTGAAAACATCACAAAACAAACAAAACCATCAGTGAGGTTGA
ATGTGTATCCATGTTTGCTTGTCTCCATTTAAAGAACACCTGGAAGATCTCTAGCTGCTGGTGGAGGCGGGAGGGTGAAGGCTGCTTCATCCTTTGT
ATTTGAACTGTGTGGCTGTGGGTGTATTATCTGTTCCCAAGGACTGCACATGAAATGAGAACAATGAAGGCTTCGGTTCTGCTGAGATGAACCTCAA
TTCTCCACAGATGATCTCACCCAGAGCCAGATGGAAGTCCCCCAACATTTAAGCCTCTGTTCTGAGTGCTGGACCCAAACTCATCTTAAGGGAAAAC
CCAAAGAGCCTTTCTGTGGTTTCCTTCCACAACTGTCTTTCAATGTGTGTATGTGTGCACGTGTGTTTGCTTGTGTGTGTGTGTGTGTACTATGTGC
ATGTGTGTGAGCATGTGTGTATATGTATGTGCATGAGTGTGTGTGTGTGTGTGTATACATGTGAGCATGTATGTATAGGCCAGAGGTTAGTGATGGG
TAACTTTCTCCAGAGCTCTCCACCTTAGTTTTATTGTCTGTGTGTGAGCGGCTGTGGGCACTGTACACCACAGCACATGTGTGGAGGTCAGAGAACA
ACTTGTGGCTGTCACTTCTCTCCCTCTGTGTGGGTCTAAGACCTGGATCCAGCGCTTTCGCTTGCTTGAGACATCTCGTTGACTAGCTCCTCCGTTG
TTAACACAGGGTCTCACTATGTAGGTCCAGTTATCTTGGAATTTGCTCTGTAGACCAGGCCGGCATCAAACTCAGAGATAGACCTGCCTTTGCCTCC
AGAGTACTGGGATTAAAGGTGTGCACCACTACCACCCAGTTTAAAATTCTAAAAAGATTTGTGGGTGCAGGGATTTGAGGAGGCCAGGAGAGACTGT
TGGATCTCTGGGAACTGGAGTTAGAGGTGGTTGTGAGCCCCCACGGGTGCTGAGAACTGAACTAGGGTGCTTGAGAGGAGCTCACTCACTGCTGAGC
CCCCACTTTAGATTTGAGTCCCTCGTACTGAAGCTGAGTTCAGTGATTTGGCTGAGCTGACCGCTCAGGGTGGGCTTTTCGATGTGCACCACAGGCC
TGGCTTTATAGGTGAGGTCATGCTTGCACAACTGGCCTTTTGGTGACTCAGCCAACTCCCCAGACCTTTTTTCTTTTGCATTTACGTGTGTGTGTGT
GTGTGTGTGTGTGTGTGTGTGTGTGTGTGTACATGAAGTGAGAGGGGGCAGTCTGGGAAGGGTCAGTGGACAACTTAAGGGAGTTGGTTTTGCCTTC
CATCATGATGGTCTAGAGACTAAATTCAGCCCCTCAGCCTGGGAGCGTGCCTTTACCCTCCGAGCTCCCAGCTTTGGGTTTTTGAGGCAAGGTTTCA
TATAGCTTAGGCTGGCCTGGCATTCAAGGTCATCTCAGAGGCCTAGGATGACCTTGTGTTGTCCGTTCTCCGGCCTTTACCTCTGGTGTGCTGGCGT
TTGCCTTACCACACCCAGGCATCCCCCATAGCTTAAGCCTAAAGCTCTCGGATGGCCAGCACGCCCATGCCCTGACGTGAAGTTCAGTGACTAATCA
ATACAGAGCGTTCACGGAGCCCTGACACATAGCAGCATTGACTTAACCTTAACAGCCTGGTGTGTAATGAGGCCAACTTGTCCGATTCTGCCCACGA
TGCGATGCTCTGGGTTAAACAGGTTTGAACAGAGTACCGGATGGCAGGGAGGGCACCCTCAGTACAGGGTTTTGGCTGTTACTGCTTCCAGTCTCCT
GACCTGCTCCCCCAAGGCTGGTCTTAAACTCAAAGCAATCCTCCTGTCCCTGCTTCTCAAGTTCTGGAATTACAAGTGTGACTCACTGTACCCAGGA
ATATGTGTCTTTATTACAGAGGAAGGAGAGCTCAGAGAGTGAGAGTAACCTGTGCAAAGTCCCACAGCAGGAGAGGGTGGCTGGCTATCGGCCGTGA
ACTTGGTGACCTTTCCTGTGACCTCTAAGGGAGGAACTAGATGTCCCAGCAAGGATTCTAGGGACTTGTCAGGACTGACCTTTGATGATGGGAAAGC
CATGCCATCTCCCCTTCCCCTTTGATGGTCGCTGGAGGGGTTTGCCTGGGACAGAGATGTGTTTGGACACAGATCACCGTGAAGACTGGTCAGTTTC
AGTACCCTGCCCAGCCTGTGGCTGGGAAGGGCCAGGGGCCCAGATCTTGTGGGCAGCCTGGGGTAGGGCCAGCATTCCAGGGGCCTGCCTGGCATGT
GGGGAATGTCCCAGGAGAAGTCTCAGTCCTCCAGAAACTCAAAGGGAAGTGTTCTTACGTGGGGTGGTGTGTGGTGGGAGGTGGGAGGTGGTGTTTG
TGGGAATGTCCCTCTGGTCTGGAACCCTGAGGGGCTGCAGTTAGGAGGTACATTGGGGCTGTGCCATTCTAGGGAGCTGACCTGCTTTTCTGATGTC
TTCATAAACAGAGACCACTATTCCTGCCTTACCTACCCTGCTTCCTGGTAGACCAGCACCCCGGGGCTTGAGCATCACCACAGTAGGGACTTTTGTG
GGCCACTGACTTGGGCCACAGTTACCACAATTTATTTAGAGTCAGCAAGGTGGCTGTACTCTATGCTGACCCGGGAATAGAAATCTCGGTTTCTGCT
CAGGAGGCCCAGGCTACAAGGCCTTGAAAAACCCTGAGGCGGGCGGGGCAGGTAGGGCTCTGCATGCGCCTCTCTAGAAATTAACCTCCTTTCTACC
CCAGACCCTTCTGGCGTCTCTGGCTTCTTCAAGGACCGGCTCTAAGGCTACTTCCTGGTACACCCTGGACCCTCCCTCCATCCTGAAGGGATCATTA
TCTTAAACCTGGTCCTTCCCATCGACTGGGTTTGGGGGTAGGGGTGTTTCAGGATGGCCTCAGAGAGCAGGAGAAAGTTGGAGATCAGCCAACTACT
GCAGAGCACGACTGTGGAGGAACAGTTAAATGGACTAGCAAACACGAACGGGCCAGAGGCAGAAGGACTGCTATGAGTTCGAGTCCAGCCTGAGCCA
CACAGATACTTCAAGCCTAGTCTGAGTTACAGAGTGAGACCCGGTCTTAAACCCCAGAAAAACACTCCGGGGAAGCGCAGGGGACCGGGCGCGCGCT
GCAGAGCCCCCTCCCCGGCAGGCCCGGGTAGGGGCGTGGCCACGGTGACGTCATCCTCCTATAAAACCCTGGGCGCCGCCGGGCTGGCTTTGTGGAG
AACTGCAGCGGGCTAAGCCGTGTTGAACAAAGGAGGTCGGGCACAGCTATCCAAGCTCCCGGGGCCACCGGGCCGCCCTCCGCCACCATGACCGCCA
ACGGCACGGCAGAGGCTGTGCAGATTCIGTTCGGGCTCATCAGCTGCGGCAACAAGTACCTGACAGCCGAGGCGTTCGGGTTCAAGGTGAACGCATC
CGCTAGTAGCTTGAAAAAGAAGCAGATCTGGACGCTGGAGCAACCTCCCGATGAGGCGGGCAGCGCGGCCGTGTGTCTGCGCAGCCACCTGGGTCGC
TACCTGGCCGCCGACAAGGACGGCAACGTGACCTGCGAGCGCGAGGTGCCCGACGGCGACTGCCGCTTTCTCGTCGTGGCGCACGACGACGGCCGCT
GCTCGCTGCAGTCCGAGGCTCACCGGCGCTACTTTGGCGGCACCGAGGACCGCCTGTCCTGCTTCGCGCAGAGCGTGTCGCCGGCCGAGAAGTGGAG
CGTGCACATCGCCATGCACCCGCAGGTTAACATCTACAGCGTTACCCGCAAGCGCTACGCGCATCTGAGCGCGCGGCCGGCCGACGAGATCGCGGTA
GACCGCGACGTGCCTTGGGGCGTCGACTCGCTCATCACCTTGGCCTTCCAGGACCAACGCTACAGTGTGCAGACGTCCGACCACCGCTTCCTGCGCC
ACGACGGGCGCCTTGTGGCACGGCCGGAGCCCGCCACGGGCTTCACGCTGGAGTTCCGCTCCGGCAAGGTGGCCTTTCGCGACTGCGAAGGTCGCTA
CCTGGCTCCGTCCGGGCCCAGCGGCACCCTCAAGGCTGGCAAGGCCACCAAGGTGGGCAAAGATGAGCTCTTCGCCCTGGAACAGAGCTGCGCTCAG
GTGGTGCTGCAGGCGGCCAACGAGAGGAACGTGTCCACGCGCCAGGGTGAGTTGGGGACATGTCCCCTCCCTTGGGTCCTGACCCAGTGCACAAAAG
CATCTCTGCACTCCTTCTATTTCCCTATTTCCTCCTGGGCTCCCTTTAGGCTCACAGGCCTCTGGGCTCCGCCATGGGGAGGTGGGCTTCTATTTTG
AAAGACCTGATCTCACCAGATAGCCTTGGCTATCCTGGAACTCGCTATGTAGACTGGGCTGGCCCCGAACTCGCAGAAATCTTCCTTCCTGTCTCTT
GTGCTGGGAATAAAGGCATGTACCACCCCTCTGTTCAGGACGTAGGCTTCTTCTGCTGGGAGAAGGTGACGTGGGGAAGTGGGTAAGGCCTAACACA
TTCTAAAGCAATACTGTCCACCACCACCCCCCCACCCCCCCACCCCCCCGGCTGGGGGCGTGGGACTCCATAGACCTAGCTTAGATCTTCTCCGTTT
CCTCTTCCTTCCCAGTCTGAGCCATCCCCATTCATGAGCCGATTCTTTAGGATAGGCTGTCCCAGGTCTTGGAGAGGAAGGGTTCCCCCTACACCCC
CCCTTGTTCATTTCCTCGTGCGTCCTCTCCCGTTTCGCGGGCCTTTGGCTGGAGGGCCAGGGGACAGTCACCCCTGCCCTCCCCGCCTTAAATGGCG
AGATAGAGGAGGTTGGAGGGGCTTTTGATCCCGGCGGGGTGTGCTGGGCGGGGTCGGCCCTGCGACTCTTGGTGGTGGTGGGGTTGTCCGTAGGGGC
TTCCCCTCCCCCACTCCATCTCCCCTCGCTAAGGCCTGGCGCTCCGGGCCGCGGCCTTTGTGAGCAGGGGGGCGGGTCGCCACGGCTGGGCCCTCCC
TCGTCCCCTTTGTCCTGCTAAGGCTCTGCAGATGGGAAAACCAGATGCGGCGGAGCTGGGGGAGGGGATCGGCTTAGGCGGGTCCCTCTGCGGAAGA
GCCACCCCCAACCCTCCAGGCGTTGTCCCTGGTCCAGGGTTCATTGAGAAGGCGCGGAGTGAAGCCGCCTGGCTCTTCCTTCGCACCCCGGCTGGCA
GGCGGCGTGGCGCGGGTGGCTGCGCGCCAGGCGCCCTCCCTGCCCTCCTATGCACGCCTTGCGGTCCGGCACTCGGCGGAGTGTCCCTCCAAAAGCC
TCTACTCTCGAGTGAACCGGTCCCCAAGGCCTCTCTGGCTGGGAACAACCGTTTGCAGCTCTATTTGTCCCCATCCAGGGCCTCCAGCAGGCTGGCA
GGCCATTTCCGACCGGCATGGGCATGGTACAAGTAGGCGCCTCCTTTCTGGCGTACCCCCGACTCTTCAAAGGGTCTTTGCTGCCTGCTGGTAGGGG
CTTGTGAGATCTCGCTTCCTTGGTTGGGAAACTGAACCCCAGGCTTTGGAGATAGCTGGATGGGAGCCAGTCTGTGGGGAGGAAGACCCCCCTCCCC
CAATGCAGAGGGCAAAGCCTTGGGTGGGGCCTGCTTGAGTTTGGCTGAATAACGCTCAGAAAAGTGCGACCATTTGCTTACTGTTACACAGTAAGTC
AGCACCATGCTGAACTGGGTGTACTTGATGCCAGAATGTTCTCTCATTATACACACACACACACACACACACACACACACACACACACACCAATGAA
CCATGGCCTCTCTTGTTTCACAGTGCTGGGCGAGGGGGTCAGAGTTCCCACCTGTTTAGCAAACATTGAGATACGAGCTGCGGTAAACGTCTCTTGG
GAATGTGACTGGTATGTAAAGCAGAAGATTGGGGGTACAGGATCGGGGTAGTAGATTATAAAGATCTGGGGTCCCTTCTGAGGACCCAGGAGCTGCC
CCAGGAGAGTGAATAGAAATGAGCATGCACCAAGTTTATGGACTGGTTTTGCAGGCAGGGGCCTCAGCAAAGGCCCGGAGGCAGGGAGTTGCAAATA
GAGAAGCCCTGCATAGCTAGGAGGAAGGCTGGGATTGGAGGGAATCCTGCAGATGACGGGTACTCCAGAGCCTAGAGAGACCAGCTCTCTGCTTACA
GAGCTGTGGCCGAGGCTTGTGCCCTGGGGAAGGTTAGGCAAGGCACAGGTGACTTGGGCCCTTGGGGCCTGAAAGGAGCTAGAGGGTGTGTTTTGCG
AGCCTGGACAGGTACCCTTTTCTGAGGGTGCCATGATTGTGCATGATTCACACTGATGCGGCAGGAGGGGGCGGGAATTGAAGGGGTGAGAATCTGA
CGGGAACTTGGGGTCACACACAGTCAGACCTCTTCTGCCCTCCTATGTCTGGGTCCAAGTCTCATTCCCGAGTCAGCCAACTCCTCCCCCGGCTTTT
TGAGGGTGAGCTGTTGGGTGGGTGTGCCGGGGAGGCCATTTTGGGCAGGCTTGCGTGGCAGGGAGGCGGGGCTGGGCTTCGGGGAAACACGTGTTGA
ACTGCTAGCCAGGAGTAGGGAGAGGGAAGGCTCAATCTGGTCTGGGGTTGTGAATCTTCCATGCAGATTTGCTGCTTCCGGCTTCCTGGCCTCAGTT
TCCCCATCTGAAAGCACAGAGGTACATTGAGCCAGGATCTTCCTGGGAGGTGGAGATCCTATGAGAGTAGGATGGGGGGTAGTGGGTAGGGTCTTTT
CCTCCTTGCTTCTCATGACCCTCCCAAGCCCCTGCTGGCTCAAGGAGACCCCGTGCTATGTCTCCCCACACTGCCCACCCTGGCTTACTTGTTGGGT
TCTAATGCTTTCAAAGAATTTTGTCCCCATCATTTCTAGGGCGGCTGGCTAAGAATGAGTGACAGGTGAGGCTGGGCTTTGTAGGGTATCCTTTTGA
CGGCTTCCCTCTTGGGTCGAGGAATGGCTTAGTTTTGAGCACTGTACGACTTTGGGCGGTGAAGTCCTGGAGGGTTCTAAACTAACCCGCCTTCTGG
GTTGTGAGGATTGCTTATTTCGAACAACAACGCTAGGGAGTCTGGCAAACAGCTAAGCCCAGGAAGGGGGTTGCGGGTTAGAGGACCCTCTGAGTCA
CCGCAGAGACGCCTAGGAAGCTGGAAGACAGCCACGCCCCCTCCGTGGGCTCCTAGGAGCCAGTGACTCAGGTTTTCTGTGCTGCTGCTGCTGAAAA
AAAAAAAAATCCCACAGAAAACCTGTTACCATAGCAACGCAGCTCCAATATGATTCCAAGGCCAAGTGGGGCCTTTCCCCCTGGCTGAGCCTTAGAG
GGCCCATTCTGGAAGGGGGCTAACTTCCCTTTCCGGCTGAGGTGGGAGGGCCCACCATCTCCTTGGCAACAGGCCCTCACGTCCTTTCACGGTCCCA
ACAGAATTGAGAGGCGATTCTTTTTGGCAAGGGAGGGAAACTGAGGCAGAGAGCGGAGAAGACATTCCCCGAAGGGTGCCTGGAGTCCACCTTTGGT
GCCTTGGTGGCCTCTTTATTAAAAAGTGGTTGCACAGGCCCCTTTGTTCCAGTACCTGGAGATGAGACCCACATGGTGAGGCCCTTTTTGTTCTCTG
TCTGGACGTCTCATTTTTCAGAGGTACTGGGAGGTTGAAGGTTGGTGTATGTCCACAGCACTGTGCTGTCTTGAACTCAGTGCGCATGACATCTTGG
GCCAGGGCCTCTCCTTTGTGTGAGGGAAGCTAGCATGCTGGGAAACTCCGACTCCTTTAGTCCACCCACTTTAGTTGTAGTTTTTGTCTTGTAGCCC
ATCTGGCTTCCAACTCTCCATCCTCCTGCCTCAGTCTCCAGGGTGCGGCGATGACAGGCATAGGTCACCACAGGCTTCTGGCCTCCACTTGATGGTT
GCCGGAGTGGCTGGGAGTGGCTTTTCTTTTCTTTTCTTTTCTTTTCTTTACTTTCAGGCAGGGTTTATCTGTGTAGTCCGGCTGTTCTGGAACTATG
TNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNAGAGAGAGAGATTGGGACTGACTGGTCCCAGTTCTGGCTT
TCCCTGACTGTTCCATTCCCCGCAGTGGGCACGGTTAGATCAGGTGGGGTCACTTAGGCTCGCTAGCATTGGGTGTGGCTCTTTTCTGCCCAGTATG
CTGTGCTAACTCAGCTGTCCCTGCACGTGGCAGGTCAATTACAGGTAGGCTGAGGGGACAGTGGGTGACACCCTGTATATGTGTAGCCAATTGTTCT
CTTAATATTTAGCCATCCCTAGTGTGGGCCAGGGGCAGGAGAGCCATTTCTGCCTGAGAAGGTTGAACAGACTACAGCCCAGAGCTGTGGCCTTGGG
GTGGCTGTCTAGCCTGGGATGGCCCTCCTACTCTCCTCTGTGCTTCAGTGGTAGATCCTGTGGAGGCACTGGGCCAGGGTCCTTGGGGACCCATCAT
GGAAGCCATGTCTGCTGATCCATATGTCTAGAAGGGGAGGCATCTGTCCATCCCCACTCCTGAAGTCAAGGGACCCCTTGGTCTTGCAGGGTGACCT
TGGGCCAAGGCCAGCCACCTCTGAGCTCCATCTTCTGCTTTGTGAGGTAGAGGTAAGAGCCTTTGGCATTGACAACACAGATAGAGACTCGGTTGGG
TCAGGGTGGCATTGGCTGGCATACTCTTGGGTGTAGTGTCAACTAAGGATGAGTTCCTAGGGGCAAGGCTGAAGGTGGCATCTACCATCCCATACCA
CGTTCTGCTGCCTAAGGACGCCTGAAGACAACTGTGAGACTGGCAGTGTACACTGCTTCCAAGCTGAGGACATTGTTAGAATCCCAGATCCTTGAAA
CAATGAGTTTATCTTGTTAGGACCCAAGATCCCACTCCTAGGTGGAGTCAAAGCCTTGGGAGCGGCACCTTATTGGGGCTCAGTGAGGTCATGTGAC
ATGGAAACCATGTAGTGTTACTATGTAGCAAGAAGTTAGAAAGCCATGCAACAAACCTGTAATTCCAACTCCTGAGGCAGAGGCAGGTGGATCTCTG
ATTTCAAAGGTAGCTCCAGATACACTGTGAGACCTTCTCAAAACAAGACAAGCAGACTGGAGAGATAGCTCAGAGGTTAAGAGCACTGGCTACTCTT
CTAGAGGTCCTGAGTTCAATTCCCAGCAACCAGTTGGTGGTCACAACCATCTGTAATGGGATTCGATACCCTCTTCTGGGGTGTCTGAGGAGAATGA
CAGTGTAGTCACATACGTAAAATAAATAAATCTTTAAAAAAAGAAAAGAAAAATGAAAAGGGAGGGGGGTGATGGAGACATGGCTCAGACAGTTTTT
TGTTTGTTTTGGCTTTTTGGTGTCTTTTCTTCTTTCTTTCTTTCTTTCTTTCTTTCTCTCTCTCTCTCTCTCTCTCTCTCTCTCTCTCTCTCTCTCT
CTCTCTTTCTCTATCTTTTCTCTTCTTGTCTTGTCTTGTATCTGTGTAGCAGCCCTGGCTACCCTGGAACTCACCTATAGGTCAGGCTAGCCTTGAA
CTCAGAGATCAGCCTGCCTTTGCCTTCCAATGCTGGGTTAAAGGTGTGCTCCAGCACTGTCCACTCTGGACCACTTGTTCTTGTATTCACCTAGTTC
TCACAGGGTGGCTCATGACAGTTCCAGGCGACCTGACCTCTTAGGGCACCAGGCACATGTGCGCATATTCATACACACAGGCAAGACGTTCACACAC
GATACATACAATACAAGACGTATCTTTCTAAAAATTAAAGAAAATAAATGAGAAGCGAGAGGGAAAGGAGGGAGAGCCACTGTTTGGACTGGACCAC
CACTGGCAGGGAAATGCCAGAAGGTTCAGACAGCATTCAGGGTTTGTGGCTGGTGGGGGCAGGGATAAAGGAGGAGTTTTCTAAAGGAACATGTGGT
CTGTGCGTGTGTGTGTGTGTGTGTGTGTGTGTGTGTGTGTGTGTGTGTTACTAGGGGTAGACTCCAGGGCCTTCCACCATGCTGGGCAAACAATTTA
CACTGAGCTATATTTTCGACCCCCTCTTTATTTTATTTTTATTTTATTTTATATATAGCTCTGACTGGCCTAGAACTCATTATGTAAACTAAGGTGA
CCTCAAACTCACCCATACTCAAATGAGATTCACCTGCCTCTGCCTCCTGAGTGCTAGAGACAGTCATGAAGCACCACGCCCACCTTCTCTTTAAAAA
AAAAAAAAAGCCTATTGCTTTTGTTTTTATTTTTGTCTGTGGAGGGTGGTGCCCACACCCAGTGGTGCCAGTTTGGAATCAGTTCTCCTGCCCTATT
CTACCTAGGGATCGAACGCAGGTGGGTAGGTTTGGCAGCAGCTGCTTTTACCCCACGAGCCATCTCGCTGGCCTGTAGCCCTTTGTGTGTCTGTCTG
TCTGTCTGTCTGTCTGCATGGAGTGGAGTCTCATGTAGCTCAGGCTGGCCTGCGAGTTATGATCTTCCTGACCCTGGTATCCAAGTGCGGAGACTGC
AGGCTCACTCTGCCACGCCCTTTTACACAGTTCTGGGATGGACTCTGCATGTCAAGTGAGTTATAGCCACGGCCCTATTTTATTTTATTATTTTTGA
GATCGGGCCCCACTCATCATGGAGGCTAAGGTCGGCCTCCTGAGTGGTGGGATGACAGGCGTGGGTCGATGTGCCTGGCTCCTACTCTATTCTTTTA
CTTTCACACATACCCACTACAGGAAGCGAGTAGCGAGCCCCCTTTTACCAGCACCCCGCCATGTGACATTCCGCAGAACCAGCGCCATCTGTGTGTT
TATTCAATGGTTTTGGTTGTATTTAAAGCAAATTCCCCACACTGGTGCTTCTTACCCGGAAACGCCTCCATCTGTAGCTACAAAACCAGGCCATTCC
TTACATAACGGCAGCGTTGCCTAACTGGAACCGATCTTTAAGACCCAATCCAGGCCCACGGGAGAGTTCTGCTAGGCCTTAGAACATCTTTGTGGGT
GGTTCGTTTGAAGTTTGATGGCTTCCTTCCTCCCCTGCTCCCCTTTCTCCTTGCCATTTTGGGCACTAGAGTTTAAAGAAACCCGCCTGCCACTGAA
CTAAATCCCCAGATTTTTTTTTTTTTAAGTTTTTTTTCGAGACAGGGTTTCTCTGTGTAGCCCTGGCTGTCCTGGAACTCACTCTGTAGACCAGGCT
GGCCTCGAACTCAGAAATCCACCTGCCTCTGCCTCCCGAGTGCTGGGATTAATGTCTTATGTATCCCAGACTGGTCTTGAACTTGTGGTCATGCCTG
GAGTATCCTATTTTGATTGTTTATGTGTATACCCCTAATATGGATGTCAGAGATTCATATCATATGTGCCGTCTTTGGTCATCCTCCATACTTTTGG
GGGATGTATGGGGGCAGGGATGGAGTCTAGCCTGGGCTGTGGTAACACACGCTTTTCATTCCAGCATTAAAGCAGAGATAGGTCGATCTCTGAGTTC
TAGGACAGCCTGGGCTACACAGGGAAATTCTGTCTCAAAAAAAAAAAAAAACAAAAAGAAAGAAAGAAAGGAAAAGAAATAAGAGTCTCCCTATATA
GCCCTGGCTGTCCTAGAACTTATTATGTAGACCACGCTGGTCTTGGACTCACAACAGAGATCCATCTGCCTCTGCCTCCTGAATGTCCACTCTCTGC
TTGGAGACAAGAATCGCTCAGTGGCCCGGATTCAGGGATTCAGCTAAGCTGGCTGGTCCCTGAGCTCCAGGCCTCCATCTGTCTTGGCTTCCCTGTG
ACAGGATTATCGGCTCATGCCACCGCGCCTGGCTCTTACACCATTCCTAGCGATCTGAATTTAATACTCTATGGTCAAGCCATCCTTCAATGCTCCC
TCCCCTCCTGTTGAGACAGAGTCTTACTCACTCGGTAGCCTGGGGTCCCAGAACTCACAGTTAGTCTCCCGTTTGTTTCCTGAGTGGTGAGATCGTT
CCTGGCTAGCCTTGGCTGCTGATTTGGTAGCCTGGTCCCGCCCACTGCTTAGGATTATCACTGAGGAGTTTAAGCCACACTTCAGGAAGCCCTGTGG
ATCCTGGAATCTGAGTGAGACAAGGGTCACACAGTGGCCTGACCGGCTCTGGTCTCTCTCTGCAGGAATGGACCTGTCAGCCAATCAGGATGAAGAG
ACCGATCAGGAGACCTTCCAGCTGGAGATCGACCGCGACACAAGAAAGTGTGCCTTTCGCACCCACACGGGCAAGTACTGGACACTGACGGCGACCG
GAGGTGTGCAATCCACTGCGTCCACCAAGTGAGTGACACCCTACACCCCTTCATCACCTGGCCTGGCTCTTCCCCCAGGACTGGGCAGCCTGCTCGA
TGCCCCCACGTTGCCAGCCCCTCTTCTCTTCCCCAGGAACGCCAGCTGCTACTTTGACATCGAGTGGTGTGACCGCCGGATCACTCTGAGAGCCTCC
AACGGCAAGTTTGTGACCGCCAAGAAAAATGGCCAGCTGGCCGCCTCGGTGGAGACAGCAGGTAGTCACTTGGGGGCACGTACCCTAAGCCTGTTTC
CCTAGTACCCCGTGGTCAACCATCAGTCCCACCTGGACCTCTCTGTGTGTTCAGGGAATCCCTGTAAGCTGGTGTACCCTCAGGCCAGCAGCCTGTG
ACCCTTAGCTTCTTGGTATCCCTCTCTGCTGAGCCATTCCCTGACTGGCCCATCTTTGTTGCTGTGAAGCTCACTGGCTCCCTTTCCCTGTGGCAGG
GGACTCGGAACTCTTCCTCATGAAGCTGATTAACCGCCCCATCATCGTGTTCCGGGGGGAACACGGGTTCATTGGCTGCCGCAAGGTCACGGGCACT
CTGGATGCCAACCGTTCCAGTTACGATGTCTTCCAGTTGGAATTCAATGACGGCGCCTACAACATCAAAGGTGGGTTCACTGGGTGAGGATGCACCT
GGCCATTCAAAGCCGACATTAGGGAACGGGTGTCCTATGACCGCCTGGGGTAGCCCCTCCCCCCTTGTCTTAGAACTTTCCTGGCCCAACCTGGGCA
GACAGCGAAGGTGGGAAGCAGCTAGGGAGAGTGGTCGTGGCTCCAATCTGGGAATCGGAGACAGGAGAATTATCTTTTTTTTTTTTTTGGTGTTGAG
GACAGAACCCAGGGCTTTGAGCTTATTAGGCAAGCATTCTACTGCTGAGTTAAATCCCCAACCCCAAGAATCATCTCAAAAAGAAAGAAAGAAAAGG
GAAAAAGAAGGTGGAGCCTGGTGGTGGTGGTGGCGGCGGCGGCGGCACACGCCTTTAGTCCCAGCACTCAAGAGGCAGAGGCAGGTGGGTCTCTGAG
TTCTTGCCAGCCTGCTCTACAGAATACATTCTAAGACAGCCAGAGCTATGCAGAGAAACCCTGTCTCAAAAACAAACAAAAAAAGGGGGTGGGGAGA
GAAGATAGAAGATAAGGATATTCCTTCTTAGCTAGCTGTGGGACCAAAACCAGTGAGAGGACACAGCAATCCAGAGGTGTCAGTCAGAATCAGAGCC
CTGTGCTGTGTGTGGCCTTAGCAAGGCAAACGGGACTCCTTTCTTACAGTGCTGAGGACTGGACTCAGAGCTAAGATCCAGCCCCTCCCTGGGGGAT
TCTAGGCAGGGGCTCTACCACTGAGCCACGCCCCCAGCCCCTCACTGGGGGATTCTAGGCAGGTGTTCTACCACTGAGCCACGCCCCCAGCCCCTCA
CTGGGGGATTCTAGGCAGGGGCTCTACCACTGAGCCACACCCCCAGCCCCTCACTGGGGGATTCTAGGCAGGGGCTCTGCTGCTGATATATGTCCTT
TTTGTTTGTTTACTTGTTTGTTTGTTTGTCTTATTGTGGCCCTAGCTGTCCTAGAACTTGCTTTGTAGACCAGAGATATACCTGTCTCTGCCTCCCA
AATGTTGGATTTAAAGGCATGTGCCACCATGCCCCACCCTAGCCTAGCCCTCTTATGTTTTGTAGTTTGAGATGGCATGTCAGTAGATTTCCCAGGA
TGGCCTTGAATCTAGGTAATCCTCCTGCCTCAGTTTCTTGAGTATTTGAATTATGGGACTGTACCTTACATCCAGCTGAGCCCACTCAGTTCTGGAA
GCTGACAGCGGGAGGGAGTGTTAATCCTTTGGGGACTGACAAGGTGGGGTTAACATGTAGTCTCCCTCCTCAGACTCCACGGGCAAGTACTGGACGG
TGGGTAGTGATTCCTCGGTCACCAGCAGCAGCGACACCCCTGTGGATTTCTTCCTTGAGTTCTGTGACTACAATAAGGTGGCTCTCAAGGTGGGCGG
CCGCTACCTGAAGGGGGACCACGCTGGGGTCCTGAAGGCCTGCGCGGAGACTATCGACCCCGCCTCACTCTGGGAGTACTAGGGCCACCTGCCCTCT
GCAGGCCGCTCTCGTCAGTCCCTCCTGTTATCCTTACTCATCGGGTGGCCCTGCAGCAGGTGGCAAACCCCTTGCCTTTCAAACTGGAAACCCAAGA
GAAAACGGTGCCCTTGCTGTCACCCTCTGTGGACCCCTTTTCCCTAACTCACTGCTCCCCATGGGTCGGTGGCTGCAGACTGTCCCCAGGAGGGACT
CTGGTTCCCTCTGTCCCCTTCTTTCCATGGGGAACTCTGGCACCTTTCTTCTGACCTCAGTCAACTGTGAGCCTTATTTCCCCCCAGGAAGTGGCCT
AGGAGAAGCTACAGGGCCTAGGGACTTACCCTGAGCTTGTAACTGGAAGACCCCGTCCCTATCCCCGCTCCCGCCCCCACCCCACCCCACCCCTGCT
CTGGCCCCAGCCTCTGGAGGCCAGCCTTTTGGCGGGACTGAAGCCGGGCATGGCCAACCTTGCCCACAAGTGTTTTTCTGGATCTTGGCTGGAAGGC
AGTCTGTCCCATCCTGCAGTGTTTGGGCCTGGCTCTTTGACTCAAAGCTAGCTAGGTGGCACGCCGTGTCGCTCCTGCACATTCTGGAAGGGGCGGG
CCTCTCACCCACCTCATTCCTTTTCCCCCTGGCCTGACTGGAAGCAGAAAAATGACCAAATCAGTATTTTTTTTTTTTTTCTTTAAGGAAATGTTAC
TGTTGAAAGGCCCTAGGCAAGCCTGCCCTGTTGGTTGTAGTCGTGAGTGGTCTTGGGGGGAGATGCTTGGCTCCTGTCCCTGCCTCCCCAGCGGGTT
CCCTCCCTCCCTCCTGCCTGACCACCCCAGCTCTGGCTCTGTGATTGGTGCTCCACGTCTTCCCAGACACCTCGGGGCTCCTGGGCGGGAGAAAGCC
GGATGTGCCCCTCCCTGGGAGCCCTGGAGTAAACCTCAGGGGGCCCTTTCCCAATCACCCCCTTCCACCGACCCCTCAACACCATGCATCTCACTCT
GGGTGTCTCGCTCCTTTATTTTTTTGTAACTGTCATTTCTATAACTCTGAAGACCCATGATAGTAAGCTTTGAACTGGAAAATAAAGTAAAATCAAG
TCTGCGGCCCGTGTGTGTCTCAGGGGAAGTGGATGCTGGAGTGGGCAGAGGGCCGGCTGGGAGGGAGGCAGCGCTGATTGATTGGCAGCCCGCAGAC
TCTTGTTTCGGTCTCGGACCCTAGCGGCTTCAGCATATGCCTTAGTAACTCTGGACTGAGAAATGGCTGACACGAGGTTCCCGAGCCTTACAAGCTG
GGCAGGCTAGAGGGTTCAGCAGGTAAAGTTGTGTGCTGTCAAGCTTGATCAGACTGAGTCCCCAGCACCGTGTGAAAAGTTGGGCACAACGGTGTAC
ACCCACCCCAGGGCCTCTGGCAAGCACAGAGACAAGAGGATCCTTGGAGCCTGCTGGCGGCCCAACCTTGCCCAATCTGTGATTCCCATTGAGTGAG
CCATAAGGTGGAGATGGGTTTTGGAAGATACCCCACATAGGCCTGCGGCCCCCCACACCCGGAAATAAAAACAAGCCAGGCTTGTCATCCCAGTTAT
TCGGGGAGCTGAGGCAGGAGGATCACAGATTTAAGACTGCCCAGGTTGAAGAGTAAGTTCAAGGGCAGCCTGGACATCTTGCTAGATTGTTTTTTTG
TTGGTTGGTTGGTTTTTGTTTTGTTCTTTTTTTCGAGACAGGGTTTCTCTGTGTAGCCCTGGCTGTCCTGGAACTCACTCTGTTGACCAGGCTGGCC
TCGAACTCAGAAATCCGCCTGCCTTTGCCTCCCGAGTGCTAGGATTAAAGGTGTGCGCCACCATGCCAGGCTTAGATGTTTTAAATGAAGGGCTGTG
GAGTGTGGTTCACGGGTAGCCTCGCTCAGACAGGCTGGGGCAGGAGAAATGGATGGCTCAGCATTGGCTGCTGCCTTAGGGTTTCCATTGCTGTGAA
CAGACACCATGACCTGGCAGCTCTTCTGAAGGACATTGGACTGGGGCTGGCTTACAGGTTCATCAAGGCAGGAAGCAAAGCACAGGCATGGTGCAGG
AGGGGCTCAAGTCTACGACCCGGGTCTCAAAGCCCACCCCCACAGTGACAGACTTTCTCCAACAAGGCCACACCCACTCCATCAAGGCCACACCTCC
TAGTAGTGCCACTCCTTGAGCCAAGCTTATTCAAACCACCAATCACAACTGCTTTTCTAGAGGACCCAGGTTCAATTCCCAGCATCCACATGACAGC
TTACAATTGTAACTACAATTCCAGGGGATCCAACACCCTTACACAGGCACACTTACAGGAAAAGCACCAATCAGTGCACATAAAAATACATAAATCG
TTAGAGAGAAGAGACCATTAGCTACCTTCACCCTTAACAGGCAAGCTGAGGTCCTGGGAGTGTGTTAGAGGTCCGCAGCTTGGAGTCTATAGGGTAG
TGGCATCGTCTATACGTTCCTTCCTTGACAGGTAGGTACTTAGCTGAGACCTCTGGGGTATCCCAGAGACCCATTTTACAGAGGGAGGCACTGAGGC
GGAGGGAAAAGACAACCTTGTTTGCTGGCTCAGAAGAGTGCCAGTGGGGTACCCACCCACAAGGCTCAGCATCCCGAGGCACTGTGGAGGCAGTTAC
AAAGCCGCATTGATTGGCTGCTGATGACGAAATGAAGAGCCCCGCGGTGTGATTGGCGGTCCCAGAGGGAAGGCATGTGGGGAGATTGACAGGGGCA
GATGGCCACAAAGGGTCCAGTGACTTCCCGCAGGGTGGAAGCTCTGGGCCCAAGCCAGTAAAGTGTCAATGACATGGAGAGACAGATGAATCCTTCC
CAGTTGAATTCTCAGGCTCTGGTGGGGCCGCGCTGGGGAGAAAAGACACCCTGTCCGTTACAACCACCAAGCGCAGCCAGGCTACGTGATGACTGGG
GGAAGTCCCAGACGTGACTTCCTGCAGCACTGGAGGGGAGGGTCCGACGGAGGAGGCTGAGAGTCTGTTGGAGGCAGCCGAGGATCGGAGGGAAGAG
GCACAGGTCTTACATGGGAGGCATAAAGTCTGGTGGGAGTCCAAGGGAGGACGGATGTAGGGTGATGGGAAGAAAGGACCTAATCCAGAAAAAGGAA
CAGAAGAGCCACCTACCTGGTTAGGGGACGCTGGCCCTCCGCAGGGAAAGCTGTCAAAGAAAGGCCTGGCTAATGAGACCATGTCGTGGGGGGCCCA
GGCAACTGTGGAGGCATCAGACACCTTGGGGCAAGGAGGGCACCCATGAGCTAGTACACACCACTAAGTATCTCAGAGTCCTGCACACACACTCATT
GACACAGGTGGGTGAGCAGCACCCACGGGCAGCTGAAGCTGAGTATCCTGCCCGCCCCTTTGTTTCTTCCAACTGAGCAGGGACAAGAAGGATCCCT
ATGAATATGGCTTTTTTTGTTTGTTTGTTTGGTTGGTTGGGTTTTTGACTTGGTTTGGTTTTTCCAGACAGGGAGTAAAGACATACACCACCATAAC
CCAGCTTTGTAAATCTGGTTTTTGTGTGTGAACCTATGAATTCCACTCCTGTTGCACTGATGGGAAATTGAGGCAAAGTCTCAAAGAAGCTGACAGC
ACAGGCTAGTGTGTTCATTTTCTCTCTACAAAAAGAGGAAAAATTGGGGCTGGTGAGATGGCTCAGCGGTTAAGAGCATTGACTGCTCTTCCAAAGC
AGGTCCCGAGTTCAAATCCCAGCAACCACATGGTGGCTCACAACCATCTATAATGAGATCTGAGACCCTCTTCTGGGGTATCTGAAGACAGCTACAG
TGTACTTAGATATAATAAATAAATAAATCTTTAAAAAAAGGAAAAATTATTTATTATTTTATTATTAAATTAATTGTTGTATTTATTTATTTTATTA
ATCTATATTTAATTTATTTTTTTATTTTGTGTGCATGAGTGTTTTGCCTGCATGTGTGTGCACTACCTGTGTGTCCACTGCCTATGGAGGTCAGAAG
AAGGCGTTGGGTCCGGGAACTGGGGTTGTATGGTTGTTAGCCGCCGCGTTCATGCTGGTTCTCTGCGAGGCGGCACTTAGTCCTGAGCCATCTTCTG
TCAGCTCCTCTACTTTCTTTTAGACAGGGTCTCCTGTAGCTCAGGCTGGGCTTCAGCTTGCCTGGTGGTTAAGGGTGACTAAGTTAAATCATCTTAC
ATTTCTGCCTCACTTCCACCTCCTGAGTGCTGGGCTTGCAGGCATGTGCCACTGGGATGGGTTTTGTTTCCTGCTGGCTTCTCACCCCGTAGCTCCG
TGTGTTGGGTCAGGGCCAGCCCTGTGCCCACTGAGCCATTAACCCTACTGAGCCCACTGTAGGGAAAAACCATAGAGGAGCTTCTATGACCCTCATG
GGTCCCTCCAGGTCTGTCAGAGTTCAGGAGGCATGGATCTCTCTGGCAGGGAGCTGTTGACTTTAGCCTTGGAATTTCCCAACCAAGTCTACACTGA
GCACCCTGTCGACGACCTGCTTCTGACTCCTCCTGTGAGGCTCTTTTGGAGTGATCTTAGTCCCCACAGGGCCCCCATCTTCTCCAGAGCTGAGTGG
CCTCTCCTACTGGACAGAGAAGAGCTGTCCTGACTTGGTTAAAATGTTGAATGCAAGGAGCGGAGGAAACGGCCCAGCTGGTGAAGTGTTTGGCTCG
ATCCCCAGAGCTGTGGAGTTCCCAACAGGATGGGGCTCACTGGCTGCCCTGATCAGCAAGCTCAGAAACCAAGATGGATAGCTCCTAAGGACTGGAA
TTGGAGGATGACCCCTGGTGACAGACACACTTGCATATGCATCTGTCTGCACACATACACATATGCATGTAAAACCCTGATCCACGTGGTAGTGCCT
GACTGTAATCCCAGTACTTGGGAGACAGGAACAGAGAATCACCTTCAGTTTGAGGCCAACCTCGTCTACATATCCAGCACCTTAGGAGACTCTGTTT
CCAACTCTTCAGGGTGGCTCACATCTCTAATCTCAACCCTCAAGAAGCCAAGACTGGAAGATGCCTAGAGAGGAAGGTTTGCTTGGGTTCAGTGCCA
GCCTCCAGAGGCAGAGGCAAGCAGATTTCTGAGTTCGAGGCCAGCCTCTACAGAGTGAGTTCCAGGACAGCCAGGCAGCCAGGGCTATACAGAGCAA
CCCTGTCTCAAACAACAACAATAACAACAACAACACAACAAGAAAACTTGAGTGTGCGTGTGCGTGTGCGTGTGCGTGTGTGTGTGTGTGTGTGTGT
GTGTGTGTTTACTATTTACAGTAAACGAGTCTTTGGGGAGGCAGCCAAAGTAGGCGAGGAACTCCTTGTCCCCTGCTGGGCTCTCTGGGTTGAGATC
TTGTCTTGATGGGGAGATAGGAAGGGATTCTCTGTCCCTCAGGGTGCTATCAGCAGTGGGAACAGATGGGGTCGGGAGAGAGACTGGGGGAGGGGAG
AATCAGGGTGGAGGAGGGCTGTGATTTAGGTTTACCATGAAACCGGAGTCATCACCGGTGGGAGAGGCTGGTCAGAGTTCCTCTTTTTTCCTCTTCT
TTCCCTAGGACCCAGGCCTGATCCGGTGGGGAGGGGAATGATTGGAAAATGCTGCCTTCTAGAGTCCCTAGCGAGAGCCACACCCCAGGCCTCAGCC
ATCAATGACAACCTTTCTGTTCTCACTGAAAGCAATTAAAGATCTGTAAGATGAGTCAGTGTTACCCATTGTAAGCTGCTGACTGCCTTGGTCCCAT
CCCTGGGACCCACATGGTGGAAGGAAAGAATGGACTGATGCCCTTGGACCCCCACGTGTGTGCTGTGGCACACACACACACGCATGCACACAAAAAT
AAGTAATGTAGTTCAAAATTTAGAATAAATCAGGCGTGGTGGTGCACACCTTTAATCCCAGTACTCGGGAGGCAGAAGCAAGTAGATCTCTGTGAGT
TTGAGACCAGCCTGGTCTACAGAGTGAGTTTCAAGCCAGCCATAGATAGCTACATAGCCAGATCTTGTGTTAGCCTCCTCCTTCTACCAACAGAAAT
TAAGATCACAAATAAACAGGAAAGAGGGTGGAAGAGACAACTCAGTGGCTATGAACATTTGCTGTTCTTGCAGAGAAACCAGGATTGAGTCCCGGCA
CCCACATAGTGGGTAAACATTATCTCTAGCTCAAATTCCAGAGGACCTGGCTTCTGAGAGCACGGTACACATGGGGTGTTCTCTCTCTCTCTCTCTC
TCTCACACACACACACACACACACACACACACACACACACACACGCAGGAAAAACAGAAAAGCACATAAAATAAAAACAAATATATAAAAATAGAAT
CCAAGGGGATTCAACGATGTCATGAGTCCACAGAAATCCTATCCTCCCCTTTTTCGGGGTGTTACACGGGCGGATCAAACCCAGGGNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNTGACTGCTCTTCCAAGGTCTTGAGTTCAATCCCAGCAACCACATGGTGGCTCAC
AACCATCCGTAATGAGATCTGACGCCCTCTTCTGGTGTGTCCGAAGACAGCTACAGTGTACTTACATATAATAAATAAATAAATCTTAAAAAAAAAA
GAAAGAAAGAAAGAAAGAAAGAGAGAGAGAAAGAAAGAAAGAGGTGAGGGCTGGGTGTGGTGGCGCACACCTTTAATCCCAGCACTTGGGAGGCAGA
GGCAGGTGGATCTCTGTGAGTCTGAGGCCAGCTTGGTCTACAGAGTGAGTTCCAGGACAGCCAGGGCTACACAGAGAAACCCTGTCTTGAAAGAAAG
AAAGAAAGAAAGAAAGAAAGAAAGAAAGAAAGAAAGAAAGAAAGAAAGAAAGAAAGAAAGAAAGGAAGGAAGGAAGGAAGGAAGAGGTGAATAGGAT
AAAATGAGGTCCAATAGGTTGGGGGCATGGCTCAGTGTTAGAGGGCTTGCCTAGCAAGTGAGAGGCCTGGGTTCAACCTCTGGCACTGAAGAGGGAT
GGTAGGTAGGTAGGTAGGTAGGTAGGTAGGTATCTAGATAGATAGATGATAAATAGGTAGATGGATGGTAGATATATAGATAATAGATGTGGGATAG
ATAGATGATAGAGATATACATAGATGATAGATTATAGATAGATAGATAGATAGATAGATAGATAGATAGATAGATAGATAGACAGACAAATGGTAGG
TATATAGATGATAGAAAGATGAGGGATAAATAGATGGTAGATAAGTAGATGATAGTAGGTAGATGAGAGGGAGAGAGAGAGAGAGCGAGAGAGAGCG
AGAAAGGATATAATAAGGTCATTAGGCTAATTTGATCCAAAGACAGATTGGGGTTCTTATAAGACAAGTGATCAAGCAGGCCAGATATGGGGGGAAC
TGATCTGTAATTCTAGCACCTGGGGTGGGAGATGGAGGCAGGAGGAAGGGGAGGTCGAGGGTATCCGCAGATACTTAGTGAGTTTGAGGCTATCCCC
AGCCACGTGAGACTCTGGCAAATCCTTCCGCACTAACAACAAAAATTCTGAAAGGCAAACCTCCCTTAACTCATAAAGCATGAATTGGGGCGTATGC
GGGCCTTCAGAGCCATGTGGTGTGTTTGGGAACAGACGACAGGAAACACTGTTGAGAGGACTCAGACCCAGAGAGGAAGGGACAGCAGAGTCACCAT
GTGGACAGGCGTTGGGGCTTGCTGGGCCGCTCACAGGAAGCAGCGCACACCAGAGCCATCCGCCTAGGAGCTGGGCCACAGTTTCCTGCAGAGTGGG
CCCTGACACACCCGGCCAAAGCAAGCACTGGCCTGTGGACAGAAAGCAGACACAGGACGGAGCCCGTTCCGGGGTCAGCTCACTGTACTTGAAGATG
TGAGCCCAAAGAAGTGGGGGGTGCCACGGCGTCTTTTGCAGATGTTACTATGCCAGTTGGTGTTAGACACTTAGATATATTTTTAAGAAGAGCAGTC
GGTACTCTTAACTACTGAACCATGTCTCCAGCCCCACAATGACATTCTTCAAAGAGCAGAGCTTAGAGGCACAGGCACTTGTCACCAAGCAGCAACA
TGAGTCAGATCCCTGAAAATCCACAGGCAGAAGGAGAGAGCTGACTCCTGTCCGTTGACCTTCATGTGAATACAGTGGTGTGTGGTGGTGTGTGTGT
GTGTGTGTGTGTGTGTGTGTGTGTGTGTGCATTTATGCCATGTGTGTCAAGAGTGTGAGGAGGACCCAAGAGAGTTCCTAGACTTAAAGCTGGTTNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNN
MOUSE SEQUENCE - mRNA
GAGCTAAGCCGTGTTGAACAAAGGAGGTCGGGCACAGCTATCCAAGCTCCCGGGGCCACCGGGCCGCCCTCCGCCACCATGACCGCCAACGGCACGG
CAGAGGCTGTGCAGATTCAGTTCGGGCTCATCAGCTGCGGCAACAAGTACCTGACAGCCGAGGCGTTCGGGTTCAAGGTGAACGCATCCGCTAGTAG
CTTGAAAAAGAAGCAGATCTGGACGCTGGAGCAACCTCCCGATGAGGCGGGCAGCGCGGCCGTGTGTCTGCGCACGCACCTGGGTCGCTACCTGGCC
GCCGACAAGGACGGCAACGTGACCTGCGAGCGCGAGGTGCCCGACGGCGACTGCCGCTTTCTCGTCGTGGCGCACGACGACGGCCGCTGGTCGCTGC
AGTCCGAGGCTCACCGGCGCTACTTTGGCGGCACCGAGGACCGCCTGTCCTGCTTCGCGCAGAGCGTGTCGCCGGCCGAGAAGTGGAGCGTGCACAT
CGCCATGCACCCGCAGGTTAACATCTACAGCGTTACCCGCAAGCGCTACGCGCATCTGAGCGCGCGGCCGGCCGACGAGATCGCGGTAGACCGCGAC
GTGCCTTGGGGCGTCGACTCGCTCATCACCTTGGCCTTCCAGGACCAACGCTACAGTGTGCAGACGTCCGACCACCGCTTCCTGCGCCACGACGGGC
GCCTTGTGGCACGGCCGGAGCCCGCCACGGGCTTCACGCTGGAGTTCCGCTCCGGCAAGGTGGCCTTTCGCGACTGCGAAGGTCGCTACCTGGCTCC
GTCCGGGCCCAGCGGCACCCTCAAGGCTGGCAAGGCCACCAAGGTGGGCAAAGATGAGCTCTTCGCCCTGGAACAGAGCTGCGCTGAGGTGGTGCTG
CAGGCGGCCAACGAGGGGAACGTGTCCACGCGCCAGGGAATGGACCTGTCAGCCAATCAGGATGAAGAGACCGATCAGGAGACCTTCCAGCTGGAGA
TCGACCGCGACACAAGAAAGTGTGCCTTTCGCACCCACACGGGCAAGTACTGGACACTGACGGCGACCGGAGGTGTGCAATCCACTGCGTCCACCAA
GAACGCCAGCTGCTACTTTGACATCGAGTGGTGTGACCGCCGGATCACTCTGAGAGCCTCCAACGGCAAGTTTGTGACCGCCAAGAAAAATGGCCAC
GTGGCCGCCTCGGVGGAGACAGCAGGGGACTCGGAACTCTTCCTCATGAAGCTGATTAACCGCCCCATCATTGCGTTCCGGGGGGAACACGGGTTCA
TTGCGTGCCGCAAGGTCACGGGCACTCTGGATGCCAACCGTTCCAGTTACGATGTCTTCCAGTTGGAATTCAATGACGGCGCCTACAACATCAAAGA
CTCCACGGGCAAGTACTGGACGGTGGGTAGTGATTCCTCGGTCACCAGCAGCAGCGACACCCCTGTGGATTTCTTCCTTGAGTTCTGTGACTACAAT
AAGGTGGCTCTCAAGGTGGGCGGCCGCTACCTGAAGGGGGACCACGCTGGGGTCCTGAAGGCCTGCGCGGAGACTATCGACCCCGCCTCACTCTGGG
AGTACTAGGGCCACCTGCCTCTGCAGCCGCTCTCGTCAGTCCTCCTGTTATCCTTACTCATCGGGTGGCCTGCAGGAGGTGGCAAACCCCTTGCCTT
TCAAACTGGAAACCCAAGAGAAAACGGTGCCCTTGCTGTCACCCTCTGTGGACCCCTTTTCCCTAACTCACTGCTCCCCATGGGTCGGTGGCTGCAG
ACTGTCCCCAGGAGGACTCTGGTTCCCTCTGTCCCCTTCTTTCCATGGGGAACTCTGGCACCTTTCTTCTGACCTCAGTCAACTCTGAGCCTTATTT
CCCCCCAGGAAGTGGCCTAGGAGAAGCTACAGGGCCTAGGGACTTACCCTGAGCTTGTAACTGGAAGACCCCGTCCCTATCCCCGCTCCCGCCCCCA
CCCCACCCCACCCCTGCTCTGGCCCCAGCCTCTGGAGGCCAGCCTTTTGGCGGGACTGAAGCCGGGCATGGCCAACCTTGCCCACAAGTGTTTTTCT
GGATCTTGGCTGGAAGGCAGTCTGTCCCATCCTGCAGTGTTTGGGCCTGGCTCTTTGACTCAAAGCTAGCTAGGTGGCACTCCGTGTCGCTCCTGCA
CATTCTGGAAGGGGCGGGCCTCTCACCCACCTCATTCCTTTTCCCCCTGGCCTGACTGGAAGCAGAAAAATGACCAAATCAGTATTTTTTTTTTTTC
TTTAAGGAAATGTTACTGTTGAAAGGCCCTAGGCAAGCCTGCCCTGTTGGTTGTAGTCGTGAGTGGTCTTGGGGGGAGATGCTTGGCTCCTGTCCCT
GCCTCCCCAGCGGTTCCCTCCCTCCCTCCTGCCTGACCACCCCAGCTCTGGCTCTGTGATTGGTGCTCCACGTCTCCAGACACCTCGGGGCTCCTGG
GCGGAGAAAGCCGATGTGCCCCTCCCTGGGAGCCCTGAGTAAACCTCAGGGGGCCCTTTCCCAATCACCCCTCCACCGACCCCTCAACACCATGCAT
CTCACTCTGGGTGTACTCGCTCACATTTATTTTTTTGTAACTGTCATTTCTATAACTCTGAAGACCCATGATAGTAAGCTTTGAACTGGAAAATAAA
GTAAAATCAAGTCTG
MOUSE SEQUENCE - CODING
ATGACCGCCAACGGCACGGCAGAGGCTGTGCAGATTCAGTTCGGGCTCATCAGCTGCGGCAACAAGTACCTGACAGCCGAGGCGTTCGGGTTCAAGG
TGAACGCATCCGCTAGTAGCTTGAAAAAGAAGCAGATCTGGACGCTGGAGCAACCTCCCGATGAGGCGGGCAGCGCGGCCGTGTGTCTGCGCACGCA
CCTGGGTCGCTACCTGGCCGCCGACAAGGACGGCAACGTGACCTGCGAGCGCGAGGTGCCCGACGGCGACTGCCGCTTTCTCGTCGTGGCGCACGAC
GACGGCCGCTGGTCGCTGCAGTCCGAGGCTCACCGGCGCTACTTTGGCGGCACCGAGGACCGCCTGTCCTGCTTCGCGCAGAGCGTGTCGCCGGCCG
AGAAGTGGAGCGTGCACATCGCCATGCACCCGCAGGTTAACATCTACAGCGTTACCCGCAAGCGCTACGCGCATCTGAGCGCGCGGCCGGCCGACGA
GATCGCGGTAGACCGCGACGTGCCTTGGGGCGTCGACTCGCTCATCACCTTGGCCTTCCAGGACCAACGCTACAGTGTGCAGACGTCCGACCACCGC
TTCCTGCGCCACGACGGGCGCCTTGTGGCACGGCCGGAGCCCGCCACGGGCTTCACGCTGGAGTTCCGCTCCGGCAAGGTGGCCTTTCGCGACTGCG
AAGGTCGCTACCTGGCTCCGTCCGGGCCCAGCGGCACCCTCAAGGCTGGCAAGGCCACCAAGGTGGGCAAAGATGAGCTCTTCGCCCTGGAACAGAG
CTGCGCTGAGGTGGTGCTGCAGGCGGCCAACGAGGGGAACGTGTCCACGCGCCAGGGAATGGACCTGTCAGCCAATCAGGATGAAGAGACCGATCAG
GAGACCTTCCAGCTGGAGATCGACCGCGACACAAGAAAGTGTGCCTTTCGCACCCACACGGGCAAGTACTGGACACTGACGGCGACCGGAGGTGTGC
AATCCACTGCGTCCACCAAGAACGCCAGCTGCTACTTTGACATCGAGTGGTGTGACCGCCGGATCACTCTGAGAGCCTCCAACGGCAAGTTTGTGAC
CGCCAAGAAAAATGGCCACGTGGCCGCCTCGGTGGAGACAGCAGGGGACTCGGAACTCTTCCTCATGAAGCTGATTAACCGCCCCATCATTGCGTTC
CGGGGGGAACACGGGTTCATTGCGTGCCGCAAGGTCACGGGCACTCTGGATGCCAACCGTTCCAGTTACGATGTCTTCCAGTTGGAATTCAATGACG
GCGCCTACAACATCAAAGACTCCACGGGCAAGTACTGGACGGTGGGTAGTGATTCCTCGGTCACCAGCAGCAGCGACACCCCTGTGGATTTCTTCCT
TGAGTTCTGTGACTACAATAAGGTGGCTCTCAAGGTGGGCGGCCGCTACCTGAAGGGGGACCACGCTGGGGTCCTGAAGGCCTGCGCGGAGACTATC
GACCCCGCCTCACTCTGGGAGTACTAG
HUMAN SEQUENCE - GENOMIC
TTGCATTTGAAGCTACCATGACTGCAAGAGGGAGTTCCCGAAGGGGTCTGGGAATGGCCCAGGGAAATAGGGGTGAGGTGAGACTCCACTGCCCCGA
CAGTGCCCATGTATGAATTAGAAAAAGTGGGCCAGGCGTGGTGGCTCACACCTGTAATCCCAGCATTTTGGGAGGCTGAGTGGGCAGATCATGAGGT
CAGGAGTTCGAAACCAGCCTGGCCAACATGGTGAAACCCCATCTCTACTAAAAATACAAAAATTAGCTGGGCTTGGTGGCACACATCTGTAGTCCCA
GCTACTCAGGAGGCTGAGGCAGAAGAATCGCTTGAACCTGGGAGGTGGAGGTTGCAGTGAGCCGAGGTCATGCCACTGCACTCCAGCCTGGGTGACA
GAGTGAGACTTTGTCTCAAAAAAAAAAAAAAAAAAAAAAAGAAAAGAAAGAAAGAAAAAGAAAAAAAGTGGGCCAGGTGTGGTGGCTCATACCTGTA
ATCCCAGCACTTTGGGAGGCTGAGGTGGGAGGATTGCTTACAGCCAGGAGTTTGAGACCAGGTTGGACAACATGGTGAGACCTTGTCTTTACAAAAA
AATACAAAAACCTATCTGGGCATGGTGTTGCATTCCTTTAGTCCCAGCTACTTAGGAGGTTGAGGTGGAAGGATCACTTGAGTTAAGGGAGGAGACC
ACCCCTCATATTGTCTTATGCCCAATTTCTGCCTCCAAAGAAAGAAAAAGTAAAAACTAAAAGGCAGAAATGAAAACCACAGGCAGACAGCCCAGCG
CCACACCCTGGGCCTCGTAGTTAAAGATCGACCCCTGATCTAATCGGTGATGTTATCTATAGACTACAGACATTGTATAGAAATGCACTGTGAAAAT
CCCTATCTGGTTTTGTTCTGATCTAATTACCGGTGCATGCAGCCCCCAGTCACGTACCCCCTGCTTGCTCAATCACGACCCTCTCACGTGCACCCCC
TTAGAGTTGTGAGCCCTTAAAAGGGACAGGAATTGCTCACTCGGGGAGCTTGGCTCTTGAGACAGGAGTCTTGCTGATGCCCCTGGCCAAATAAACC
CCTTCCTTCTTTAACTCGGTGTCTGAGTTTTGTCTGCGGCTCATCCTGCTACAGAGTCTAGGAGGCAGAGGTTGCAGTAAGCCAAATTCACGCCACT
GCACTCCAGCCTGGGTGACAGAGCAAGACCCCACCAAAAAAAGAAAAGAGGCCAGGCGCAGTGGCTCACGCCCAGCTAATTTTTGTACTTTTAGTAG
AGACGGGGTTTCACCATGTTGGCCAGGCTGATCTCAAACTCCTGTCCTCAGGTGATCCGCCCACCTTGGCCTCCCAAAGTGCTGCGATAACAGGAGT
GGAGGCTCAACTTTTTAAAGAAGAAAAGGACGAATCAGGACAGGAGACAATTACAAGCTCTGTTCATTCGGAATTCTCATTGGCTTACAGAAATAAC
TTTGGCTAGTGATTGGTTATATGTTGTAGACAGACCCACAGGGTGGATGGCGTCTGTGGCCACTTGGGCATTAGCTGGTCCAGAGCCCAGAGTCCAT
GTAGCAAGTAGCTTCAGGACGTAATTATTTAGCTCAATAGAAAGTGAGATGTGACTGCTGTTACTTTTTTTTTTTTTTGAGACGGAGTTTCACTCTT
GTTGCCCAAGCTGGAGTGCAATGGCGCGATCTCGGCTCACTGCAACCTCTGTCTCCCAGGTTCAAGTGATTCTCCTGCCTCAGCCTCCCGAGTAGCT
GGGATTACAGGCGTGTGCCACCAAGCCCGGCTAATTTTTGTATTTTTAGTAGAGACAGGGTTTCACTATGTTAGCCAGGATGGTCTCGAACTCCTGA
CCTTGTCATCCACCGGCCTCGGCCTCTCAAGGTGCTAGGATTACAGGCATGAGCCACCACGCCCGGCCTTTTAACTGCTGTTACTTTTTTTTTTTTA
ATCATTTTTATTCTGCTAGTTCCATAAAAGCAATGTAAACACCAGCATTCTATACATCTTGCTGGTGAACTCACATAATGCTTAGTTCCCTGATCCT
TTGACCTCCTTGTCTTCTCCAGTTATTTTCTGTTTGGACCACTGGCCACAGGAATGGAGTGGGGTTGGGGCTTAGGAGAGAGCAATGTGGCTTTTTG
GTATGACTTGTTTCATTGACTGCTGTTACATTTTAAATGCCTTTCTGGGCCTGATAATTTAAGGGGGCTGGCATTTCTCACATCAAAGGGCAAAGGG
TTTTTTTGTTTGTTTCTCACTATTGCTCAAAAGCCACATGTGTCAAGGACAGCTCTTGTTAACGGGCAGAGAAGCTTATAGAACCTTTCCAGGGCTG
GCGCGGTTGTTCATGTCTGGGGGACAAGAGCGAAACTCCATCTCAAAAAGAAAAAAAAAAAAAAGAACATTTCCAGGCCAGGCACAGTGGCTCACAC
CTGTAAGTCCCAGCACTTTGGGAGATTGAGGTGGGAGGATCACTTGAGGCCAGGAGTTTGAGACCAGCATAGGCCACATGGCAAGAACCCAGTTTCT
ACAAAAACATTTTTTTTTTAAATTAGCTGGGCTCGGTGGTGCACACTTGTGGTCCCAGCTACTCGGGAGGCTGAGGTGGGAGAATCGCTTGAGCCTG
GGAGGCGGAGGTTGCAGTGAGCTGTGATCGCACCACTGCATTTCAGCCTGGGCAACAGAGGGAGACTCTGTCTCAAAAAAAATCAAATAAAGAAAAA
AGAACATTTCCATTATTGCAGAATGTTCTATTGGACGGCGCTGGGCCAGATGCCTGCCCCAAGCCCTGGGACACCCAAACCTGGTGAAAGTGCCAAG
CTCCCATCTGGGGGTGCTCACGGAAGGGGCCGGGAGCTGGGATTACAAGCGTGGAGGCAGGCGCCACCCCAGAGGAGGTGGGTGATGTCTGAGCCAG
AGTCTTTGGGATGAGCAGGGCTTTGGCAGGCAGCTTTGTTGGGCAGGCAACAGGCACAGCAGGTGCAATGGCATCGAGGTTGGAGAGGTGGACTGGC
GGGGAGAAGAGAAGGGAGGGTGGCAGGAGAGATGGGCAGAGGCCCCCCAGGAGCCCAGAGCCTTCAGGGCTTTGGGCCCTCTTGGGGCACTGGGGAG
CCACGGGAGAAGTGTGGGGTCGAGAGGGGCGCCCTGGATTTGACCACCTTCAGGAACCTACCTTGGCTGCCCGGGTGGGTGGGATGGAGGTGATGAA
GGTGGAGGTCGTAGCATTGTCACTGAGAGCGATGCTTATTCTGATTTTTGCCCCCGTGAGCCTCAGAAATCGGTCAGAACAGTGCTCGGGCTGGGCG
TGGTGGCTCACGCCTGGAATCCCAGCACTTTGGGAGGCCGAGGCGGGCGGATCACCTGAGGTCAGGAGTTTGAGACCAGCCTGGCCAACATGGCAAA
ACCCCGTCTCTACTAAAAACACAAAAATTAGCTGGGTGTAGTGGTGCATGCCTGTAATCCAGCTACTCCAGAGGCTGAGGCCTCCCTGGGAGGCGGA
GTGCAGTGAGCCAAGATTGTACCACCGCCCCTCCAGCCTGGGTAACAGAGTGAGACTCTGTCTCAAAACAAACAAAAAAGAACAAACCAAAACCAGT
GCTCCAATTCACCTTGCTGAATTGGAGAAGAACAGGTGCCTGGGTCCTCCCGGCTGACGGTGACGTCACTATGAGCCACCACGCCTGGCCCGGTGGG
TGTTTACAGCAGAGGTGGTTCAGGGAGGAGTGAGTGGCAGATGGTGCAGGCTCAGGGAGGAGTGAGTGGCAGGTGGGGCCTGAGGCCAGAGGCAGGA
GGACCGTTCAAGACTGAGGTGTCTTGGGCACTGGTGCCGGGGCCGGGGAGAGCCCTGGCATGCCGGGCCCCGGCCCTCAGTGAAAGCAGGATGAGGT
GGCGGCCAAGCAGGGAGGCCAAGGCCTGGCAGCAGCCGGCAGCCCCTCTCCTGGCAGGGACATCTTGGCAGGAGCCGGGCTCTGCCCAGGGCGCTAA
TGTCCTCTTAATTAGCCAGGCACTTGCCAAGCACTTGCCGCTGCGGCCTCACTTTGCAGAACGCACTTGGGTTTGGAGCAGGACCAGCATCGGCCAC
CGGGTGAGTAGGGGGGATGGCCAGGTGAGTACCATGGCCAGAAGGTGCGGCCTCTGAGTCCCCACTGAAGCCAATCCAGGCCCCGACCTCTGGTCAT
CATCAACAGAAGAGTGACAGAAACAGAAAAGATATCAAGGAACACGTTTTCAGGGCCAGATTCCACGTCCCCCACCTATTTTCCTTTGCTCCTGCCT
TGCCTGGCTCTGCCCCCTTGAACTCTTATTCACCCAGTGTAGGCATCACCTCCTCCTGAAGGCCCTCCTGGACTGCACAGTTAACTCTGGAGCCTCC
CCTGCACAGGGCAGCTCATGTATGGATCCACAGGTGAGTTCCTGTCTTTGAAGGGTTACAGTTTAGGTCCTGGAGCAGTGGTGGGGAGGTCACACCT
CCACCCACATCCCTCTTTGCAAATCCCATGGGAACTATGAGCCTGGGTCCCCCAAACTGCATGTTCCCCACTTAAAGCTGGGCCAGATCCTGTTTGC
TGTATCCAGGCCTCAGTTTCCCTGCTTGTAACCAGAAGTCCATGCAGCCAGAAATTGTCAGGGAGCTGGGCACGGTGGCTCATGCCTGTAATCCCAG
CACTTTCAGAGGCTGAGGCATGAGGTTCACTTGTGATCAGGAGTTTGGGACCCCAGCCTGGGCAATACAATGAGATCCCTGCCTCTACCAAAAAATT
AAAAACAATTACCCAGGCCTGGAGGCATACACTTGTAGTCCTAGCTATTCAAGGGAGGCTGAGGTGGGAGGATCACTTGAGTCCAGGAGTTTGTGGC
TGGGGTGAGCTCTGATTGTACCACTGCACTCCAGCCTGGGTAAGAGAATGAGACCTTGTTTTATTTTATTTTATTTTTATTTATTTTGAGATGGAGT
CTCACTCTGTCACCCAGGCTGGAGTGCAGTGGTGCAATGTCGGTTCACTGCAACCTCTGCTTCCCGGCTTCAAGAGATTCTCCCGCCTCAGCCTCCC
AAGTAGCTGGGATTACAGGCGTGTGCCACCACGCCTGGCTAATTTTTTGTGTTTTTAGTAGAGACAGGGTTTCACCATGTTTGCCAGGCTGGACTTG
AACTCCTGACCTTAGGTGATCCATGCCCCCTTGGCCTCCTAAATTGCTGGGATTATAGGCATGAGCCACCGGGCCTGGCCGAGACCCTGTTTTAAAA
AAATTAATCAGGTCCAGTGCGGTGGCTCACGCCTGTAATCCCAGCACTTTGGGAGGCCGAGGAGGGCAGATCACCTGAGATCGGGAGTTCGAGACCA
GCCTGACCAACATGCAGAAACCCCGTCTCTACTAAAAATACAAAATTAGCAGGGCGTGGTGGCGCACACCTGTAATCCCAGCTACTTGGGAGGCTGA
GGCAGGAGAATCACTTGGACCTGGGAAGCAGAGCTTGTGGTGAGCCGAGATTGCACCATTCCACTCCAGCCTGGGCAAAAAGAGTGAAACTCCATCT
CAAATAATAATAATAATAATAATAATAATAATAATAATAATAATTGGTAGAGATGTTGGTAGAGGCAGGACAGTGGGAGTGACAGCCTGGTCCTGTC
CTCCCGGGCTCTGGCTGACCATGCTGTAGTGTGGCTCAGGGCCCTGGAGGGCACAGCTGTCCTGTGGCCCTGTGGTCAGCCGGGAAGCCAGGCCTGG
CAGGACTCCTGCCCTAGAATCAGCTCCTCCCACTCCCCAGGGTCCCATGCAGGGCGCTGGCCGTACCAGGCCTCCTCGCGGGTGCCACAGAGCCAGC
GGCTGCACAGCAACAGAGACGCCATTCACAGGCTCTCGGCCACGAGGCCGCTCATTTACAGAGGGCTGAGCCGCTGACATCTGTGCGTTTGTAACAT
GGACAGCATTTCTGGTCCCATACAGACGCCTGGGCCAGCCACATTTATCTCCCCTTGAGAGAAGGGAAACGAGGCTCGGGAGGGAATGAGGGATTGG
CTTGGGGTCACACAACTTTAATGATGACCCTGGGCAGGAAGCTCCCAGGATGGCAACAGAGACTCAGGGCAGGCTGGGCATGGTGGCTCACACTTGT
CATCCCAGCCCTTTGGGAGGCAGAGGTGCAAGGATCACTCGAGCCCAGGAGCTCAAGGCCAGCCTGGGGAATATAGTGAGAACCCATCTCTAAAAAA
AAAAAAAATAGGCCGGTCGCGGTGACTCACGCCTGTAATCCCAGCACTTTGGGAGGCTGAGGCGGGTGGATCACGAGGTCAGGAGATTGAGACCATC
CTGGCTAACACGGTGAAACCCCGTCTCTACTAAAAATACAAAAAATAGGCCGGGCGTGGTGGCTCATGCCTGTAATCCCAGCACTTTGGGAGGCTGA
GGCCGGCGGATCACGAGGTCAGGAGTTCGAGATCAGCCTGACAAACATGACGAAACCCTGTCTCTACTAAAAATACAAAAATTAGCCGGGCGTGGTG
ACACGCGCCTGTAGTCCCAGCTACTCAGGAGGCTGAGGCAGGAGAATCGCTTGAACCATCGAGGTGGAGGTTGCGGTGAGCCGAGATCGCACTACTG
CACTCCAGTCTGGGAGACAGAGTGAGACTCCGTCTCAAAAAAAAAAAAAAAAATTAGCCGGGCGTGGTGGCGGGCGCCTGTAGTCCCAGCTACTCGG
GAGGCTGAGGTAGGAGAATGGCGTGAACCCGGGAGGCGGAGCTTGCAGTGAGCCGAGATCGCGCCACTGCACTAAAGCCTGGGCAACAGAGCGAGAC
TCTATCTCAAAAAAAAAAAAAAAAATTTAGCCGGGCATGGTGGTGCATGTCTGTACCCCCAGCAATTTGGGAAGCTGAGGTGGGAGGATTGCTTAAG
CCCAGGAGTTTGAGGCGGGAGTGAGCTGTGATGGCACCACTGCACTCCAGCCTGGGTGACAGAGTGAGACCCCGTCTCTCCCTATCCCCCGCAAAAA
AAAAAAAACAACTCAGGGCTTTCCCACCCCTGTCCTTGCACAGATGAAGAACCGAAGCTTCTAGAAGGGGTATATTTTGCCCCCAGGCCCCAAATCC
TGGTCTTTGGACTACAGTCAAGACCTAGCACAGGGCTCAGGCCTAAAAAAAAGCTGTTGAGGAGGGTGTGAGGCTGCAACGTTGCGGTGAGAAGGGG
GTCCCCAGGGAGGGCGCAGCAGGAAGCCCCAGGGAAGTGCCTGGGAGAGGGAGGTTGTGCAGCCAGAAGGAGCAGCCCGCAGCCTTTGGTTGTTGAC
TCCTGCTTTGTAAGTGGCAGTCTGGTTGGGTAGGACAGGGTCCGAGTCCTCACTCAGGGAACTGAGTCCAGGGTGAGCTGAGCAGCCCTTCCTGGTG
GCCTCACTTTCCCCTGCAGAGCCTGCTGCATGGTGTCCAGTGGCCAGCCTGGGAGGAGCTCAGGACTGGCCTCACCTCGTGCCACGCCCTCGTACCA
AGGTGGCTCAGATGGCACCTGGGTTCCCGAAGGGCCCAGGAACACAGCGTCCATGTCCCCATCCTTCCCCGGAGGGACTTGGGGTGGGGCCTGCAGG
AAGGATCATGTGACTTGTTCAAGGGCAGCTTGTCCTGCCCCGGACACAGAGGTGCCCATCGTAAGCGAGATGCAGGCAAGGTGAGGACAGGGCATGG
TGCCGAGCAGGAATGATTTTCGAAAATGCTACTCTGTGGTCGGGCACGGTGGCTCACTCCTGTAATCCCAGTACTTTGGGAGGCCCAGGCGGGCAGA
TCATGAGGTCAGGAGTTCAAGACCAGCCTGGCCAACATGGTGAAACCCCTTCTCTACTGAAAATACAAAAAACTACCCGGGCGTGGTGGTGGATGCC
TGTAATCCCAGCTACTCAGGAGGCTGAGGCAGGAGAATCGCTTGAACCCTGGAGGTGAAGGTTGCAATGAGCCGAGATTGCACCACTGCACTCCGAC
CTGGGCGACAGAGAAAGACTCCGTCTGAAAAACAAACAAACAAAACCCCGAGATTCTCTTTTCCCCCGTCCGGAGCTCTATGGCCATCTGGAGCTCG
TTCTGTCCACAAGGACACATTTCCTGGCAGCAGCTGTGGACCAGGGCTCACTCACTCACATCTGTACCCAATCTAGAGCAGGACAAACACCTATCAC
CTGCACTGGCAATGGACAGAGGACAGTGGCAGCCCCATCACCAGAGGCATTCAAGCCAAGGCATTTTCTATCGTCTATTTATCTATCTATCTATCTA
TCTATCTATCTATCTATCTATCTATCTAGATGAGATCTTGCTATGTTGCCCAGGTTGAACTCCTGGCCTCAAGCGATCCTCCTGCTTCAGCCTCCCA
AAGTGTTGGGATTTCAGGTGTGAACCACTGTACCTGGCTGGAGTGCAGTGGCACTGTCATAGCTCACTGCAGCCTCCACCTCCTGGGCTCAAGGAAT
CCTCTTTCCTCAGCCTCCTGAGTAGCTGGGACCACAGGCATGCACCATCACACCCAGCTAAACCTTGATTTTTTCCATAGTATATGGCTCAGGGCAG
AGCAAAGAAGCACAAAAACATGTTGGCTTGCGGTTGAGGGCTGATGGGAGGGGGTTCTGGCCAAAGCCCAAAATTAACAGCCACAACGTCAGTGTCT
GTGGGAGGGGTTGCCAGGGGCGTGCGGGTTCTGGGGCTCAAGGCCCTGCCGGTAAACCCATTTGAAGCAGGATGGCAAGAAGGTGACACCATCTTCC
CCCGCGCACTGAAAGCCCCTGGCTGGGATTGCCTGGGGCAGACACAGGCTCGGACCAGCCCCAGCAATCCCAGTTTATCAGCGAGCCGGCTGAGGGC
CCGGAGTTATCTCAGTGCCCGGCCTGAGACCTTGTGGGCAGCCTGTGGTCATGCTGGCATTCCAGGGGCCTTTTGGCATGTGGGGAATGTCCAGGAA
AAGCCTCAGCCTTCGGTGAGGCGCAGAAAAGGGAAGTGTCCCTAGAGGGGGTGGGTGAGGGCGTGGGAGGTGGTGTCTGCAGGGAATGTCCCCTTTG
GGGGAGGAGGATGGAGGGTTGGGATTCTGAGGATGGGGGGGGGGGCTGTAGCCAGCACCATGTCCCTCCTGTGTGACCAGCTCAGAGTCCCATGAAA
TTGGGGCTTGGGAGGGGAAGGGACACTGGCCTGGGAACCAGAGACCTGGGCTGGTCTGGCTCACAGTTGCTGGACCTCTGTGATCCGTGTCAAAAAA
CGAGAACACCAATTCCTGTCCTGCCCACTCACCACCAGGTGGGACCTGAACCCTGGCATCGCCAGCATTGGGAATGTCGGCCACTGACTCAACCACT
CTCCCGGAGACCTATTTGGGCCACCCGAGGCGGGTGCCTGGGCCACACGGAGGGGTCCTGGTGGTCTTCAGGGCAGCGGCTGTGGGGCTGAAGCCTC
AAGGAACCACATCTCTGCATAGGAGGGCCAGGCTGCAGGGCCTCGGAGACAACCTAGTTGGGCGTGTTGGGGTCTGTGGGTCCCAGGTCCTGGCCTC
ACCGGGTCCCCACCGCGCTGTCAGCTCCCAGCCTCTTTCCCTCGTCTGCCTCTGGGCTTCTGTAAGGCTATGTGCTCCAAGGCCACCTCCTCCAGGC
AGCCCTCAGACCCCCACCTTCGCCCAGTACCGATCTGCACCGTGGTCTCTGAAGTCTCCATCGTGACCTCAAACCTCGCTCGTCCTTGCTCCTAGCG
AGGCTTGGGGTCGGGGTGTCCGAGGTGGGGGACATCCGGGGGGGTTAGGTGGCTGGCGCGGGGAGCCGGGGTTGTGAGGGGTGATGTCCTCAGGCGG
CGGCGCTGCGGGGTGCGGCGAGGACACCGGTGAGGTGAGAGCACCGGCGGGGCAGCAGCGGGGGCCGCAGCGCCGGGTCCCTCGGCCCGGGGCCCCT
CCCGCGCGGAGCCAGGGGCGGGACAGGGGGGCGTGGCCTGGTGGCGCTGACGTCACCTCGCCTATAAAATGTCCGGGGCGCCGCTAGCTGGGCTTTG
TGGAGCGCTGCGGAGGGTGCGTGCGGGCCGCGGCAGCCGAACAAAGGAGCAGGGGCGCCGCCGCAGGGACCCGCCACCCACCTCCCGGGGCCGCGCA
GCGGCCTCTCGTCTACTGCCACCATGACCGCCAACGGCACAGCCGAGGCGGTGCAGATCCAGTTCGGCCTCATCAACTGCGGCAACAAGTACCTGAC
GGCCGAGGCGTTCGGGTTCAAGGTGAACGCGTCCGCCAGCAGCCTGAAGAAGAAGCAGATCTGGACGCTGGAGCAGCCCCCTGACGAGGCGGGCAGC
GCGGCCGTGTGCCTGCGCAGCCACCTGGGCCGCTACCTGGCGGCGGACAAGGACGGCAACGTGACCTGCGAGCGCGAGGTGCCCGGTCCCGACTGCC
GTTTCCTCATCGTGGCGCACGACGACGGTCGCTGGTCGCTGCAGTCCGAGGCGCACCGGCGCTACTTCGGCGGCACCGAGGACCGCCTGTCCTGCTT
CGCGCAGACGGTGTCCCCCGCCGAGAAGTGGAGCGTGCACATCGCCATGCACCCTCAGGTCAACATCTACAGCGTCACCCGTAAGCGCTACGCGCAC
CTGAGCGCGCGGCCGGCCGACGAGATCGCCGTGGACCGCGACGTGCCCTGGGGCGTCGACTCGCTCATCACCCTCGCCTTCCAGGACCAGCGCTACA
GCGTGCAGACCGCCGACCACCGCTTCCTGCGCCACGACGGGCGCCTGGTGGCGCGCCCCGAGCCGGCCACTGGCTACACGCTGGAGTTCCGCTCCGG
CAAGGTGGCCTTCCGCGACTGCGAGGGCCGTTACCTGGCGCCGTCGGGGCCCAGCGGCACGCTCAAGGCGGGCAAGGCCACCAAGGTGGGCAAGGAC
GAGCTCTTTCCTCTGGAGCAGAGCTGCGCCCAGGTCGTGCTGCAGGCGGCCAACGAGAGGAACGTGTCCACGCGCCAGGGTGAGTGGGGACGCTGCC
CCCGCCTCTCCTGGTCCGTGCACAAAGCGCACCCCACCCGCGCCCCTCCAGCCTCCCGCCCTTTCTCGCTCGCGGCGCCGCTGCGGTCCGGAGCACT
GCCCATTGCGCCCCCGCTAGGCACGCGGGCTACCCCGCCTGGAGGGGGCGAGGAGTGGGGCTTTGCCCATCCTCGGGTGCCGCTGCCCACCTCCCAC
CCCGGGCTGGGATCATGGGCTCCCCTAGGCCCCGCGGAGTCGCAACCGTACGTGCACCCTCCTAACCCCCCCCCCCGCCCAATCTTGGCTCTCCCCA
CGCGCGCTGATCCCTCGGATCAGCTGTCCCAGCTCTTGCGGAGTGGGAGCCCCTCACCCATTTCCTCGTGCCCCTCCCCCCGCCGGCTGTCCTGGAG
CTCGGGGGTCCGAGGCAAGGGTCGCCACCCGCAAGGGCGCGCCTCCACCCCCACCGGCAGCCTTTCGCGGGCGAGATGGGGGAGGTTAGCCAGGCCT
TTGATCCCGGCGGGGCGCGCCTCCACCTCCCCGTCTGCCCGGCCTTCCTCCACCTCCTCCCGCTGCCGGGCGGGGTCGGCCTCCGCTGGTGGGGGGG
GGCGCGGGGTGTCAGCCCTTCCCCCCAGCCCCTCCTCCCGCGTCTGCCCCGCGCTCGAGGCCGCGGCCTTTGTGAGCAGGGGGGCGGGTCGCCTCGA
CTGGGTCCTCCCTCGCCCCCTTTGTCCTGCTCCATCTCTGCAGATGGGAAAACCAGATGCGGCGGGGCGGGGGAGGGGATCGGCTTTTGCGGTTCAC
CCCTGCAGAGGAGCCCCCCGCGCCGCCCCCGGGCCCAGGGCTCGGGTCCCGAGGTTCCCCAGGAGGCGGTCTCCCTCCTCGCGCCGCGGCCCGGGAA
CGGCGTGGCGCGGATGGCGGCCCTCCAGGCACCCCGCCCTTCGCCCGCCGCGCGCGTCTCCAGGCCGGTGCGCTGAGCTCCGCTCCGCGGGCGACCG
AGGGCGGCTTCAGCGCGAGCCGGGAGACCCCAGGCCAGCTCCCTCGGGAAGCCCCTACCCTCTGGTGAACCCATCCCCTAGGGCGCTCGCCGGGAAC
AATTGACTGCAACTCGATCCGTCCCCTCCGGGGCCTCCTGCAGGCACGGCTATTTGCGACCGGCCTGTGCGCCACTCTTTCCCTTCTTTCTCAGATT
CTCCCCGAGGGGCTTTCTCTTCCTTTTGGCGTCCTCTTCCACTCCTGGCGGAGAAGCTGTTCCTTGCTTGCAGACAGAGGCCCCGCTGGAGGGAGGG
GCGTGGGGGGATCTTTTTCCCTGGATAGGAAGTCGGACCCCAGGCCTTGGAGATGGCTGGACGGGAGCCCAGTTTGTGTCCTCATCCCTCCTCTTGG
AAGGAGCCCTCCTGACGGCCCCCCTCTTGGCTGTGGGCTCTGCAGGAGGGGGAAAGACCCCCCATGGCAGTGGGGGTGAGGGTGGAGGCCTGGGTGG
GGTAATGGCCATTTTGTGCCTGAAGTTTGCTACCTTAAGTCCCAGAGAGGTGAAGCTGCTTGTCATGGGTCACACAGCAAGTGACCACAAGGAAGCA
GCATGCAGAAGTGGGTGCATTTGGCTCCAGAAACCCCGTTTCCTGTTTCCCACAGCGCATGGCGGGCAGAGCTCCTCACCTGATCAGCAGGCATTGA
GCCCTACTAAGGGGACCTGCTGAGCCATGAGTGAACCAGTGGGGTTCACGTTCTCTTGGGGACATTACGAAGTGTCAAGGAAAAGGCAGCGGGTACA
GGTTATTAGATGGCTGAGTCCTTTCTGAGCAGGACATTTGAGCTGGCCTGAAGGATGAATAGAAGGTGGCAGGTATGGGGGGGGTGCTGTGTGGACC
AGCGTTGTAGGCTGTGGGTGTCTCCTGTGCAAAGGTCTTATGGCAGGAAGGTACAGAGAAAGCCCAGTGTGGCTGGGAGGAAGCTTGTGAGGTCTGG
GCCACGGGACCTTGGGTGAGAGCTTAGTGCAGGGCTATGGTCCTTGCCTTGGGGTTGCAGCCACAGCCTCCCGTGGGGAAGAAGTCGCAGAAGTAGG
TGGCCTTGGCCCTGGGGGCCAGGAAGTTGCAGAGAGCCTGAGGGTGTTTTTGGCGAGCCTGGGCAGATAACTCCCCTCTCCTGAGAAGCTTGCTGGG
GGCGTCTGGTGTGTGATTCAGGCTGATAGGGTGGGAGGAACCAGAAATTGCAGCCAGGAGAGGTGGTGGTGATGGCTCAGCTTGGAGTTGAAAGGGA
TGTGGGACGGCGCGGGGGTGGGGGTACTTGGGGGCCGAGGTTGGGGCTCCTCTGGCCTCAGATCTTGGGGTCCTTATCTCGTTTCTATGTCAGCCAA
CTCCCGGGGGGCTTTTGGGAACTGGAGTCTGCAGGGGTGGGCGTGGGGGGCGTGTCTGCAGAGGCCATTTTGGACGGGCCTGCGTGGCAAGGGGGAG
GCGTCTGCTTCCGACACACATGCTGGGGAACGCCAGCCAGGAAGGGGAGGATCCGGACTTAGCTGGCAGGGGGGATGTGAATCATCTCTGCAGGCCT
GCACGGGCGGGCCCGGTGGGGGCAGGGTGCTCCCCTTTGAAAAACGCTGCGCCCTGCTCTTCAGCCTCAGTTTCCCCATCTGTAAAGTGTGCATTCC
AGGCCTCCTTGGGCTGGCCCAGAGCTGCCCTGGGCAGGGGCTTTCAGCCCTTCACACCAGAGGCTGGGTCAGCTGAGTGAGTGCTCCTTTGTCCTCC
CGCCATGCCCCAGGCTCCTCCCTGCTCCCCAGGATCCACACCTACCCTGGCCCCGACCCTGGGCCATGCCTCACCACACTTCCTGTCTTCTCTCACA
TCTCTCACATCTGGGAGTCCTCTCCCGCCAGCCTGTGCTTGCATCTGGGGATCCATGCCAGGGATAGGACCTGTCCTCCCTGCTGGCTTGGGACATG
CCCTCTCCCAGCCACTCTGAGGAGGGCTGACTGAGGAAGGGCTCGTCAAGCTGGGCTTTGCAGGATGTGTAAGAGTTCTCCCCACGAGGCGCAGGGC
ATTCTGAGCCCAGGGAATGGCTTGTATAAGGATGCAGAGGCATTTGAAATGGCCAAGTAGTTGCAGGAATCGACTGGAAACCGGGGTGGTAAGGTGA
AGCCATAGAACATTCCAGGCCCCCTCCCCTAAATGAGATGGAAGGAGTGCCTGTTTTGAACAAGCCAGGGGCATCTGGGGACCGTTAAGGCCTGGGG
GTGGTGATGGGGACTGGAGGGTGTGAGGCAACCAGGGGGTGCCCCTCTGAGCCACCACAGACAGAATGGTTCAGAAGGCCAGGCACACTGGCTCTCG
CCTGTAATCCAAGCACTTTGGGAGGCCAAGGAGGGAAGATGGCTTCGGCCAAGGAGGGAAGATGGCTTCAGCCCAGGAGTTCAAGGCCAGCCTGGAA
AACATGGCAAAACCTCTGTCTACAAAAAATACAAAAAATTAGCCAGGCATGGTGATGCACGCCTGTCGTCCCAGCTACTCAGGAGGCTTAGATGGGA
GGATCACTTGAGCCGGGGAGTTTGAGGCTGCAGTGAGCCGAGATCGTGCCACTGCACTCCAGCCTGGGCAACAGAACGAGACCCTGTCTTAAAAAAA
TTTTTTTTGGCCTGGTGCGGTGGCTCACGCCTGTAATGCCAGCACTTTGGGAGTCCAAGGTGGGTGGATCACCTGGAGGTCGGGAGTTTGAGACCAG
TCTGACCAACATGGAGAAACCCCATCTCTACTAAAAACACAAAAATTAGCCGGGCGTGGTGGCGCATGCCTGTAATCCCAGCTACTCTGGAGGCTGA
GGCAGGAGAATCCCTTGAACCCGGGAGGCGGAAGTTGCAGTGAGCCGAGATCGCACCATTGCACTCCAGCCTGGGCAACAAGAGTGAAACTCCATCT
CAAAAAACAAAACAAAACAAAAAAAACAACCAAAACAAACAAACAAAAAACTGTAATTAAAATAATTAAAAGAAAAAAGTTAAAAAAAAAAAAAAGA
GAGGATGGCTCAAAGGCTGGAAACAGGGAAGGCCACTGTGAGGGGAGGACAGGCAGGGCTAGACCTCTCTGGAAGGAGGAGGGAGAGGTACCCGTGG
GCCCGGCACAGGAGACTCTTAATCTCCTGGCTCCCGGGGGCTTCTGTGGGCCTGTGACTCAGGATTTCTGTGCCTCTGTTGATGAAAAGAAAAATCC
TGAAGAAAACACTGTTCCCATAGTAACACAGCTCTAATTAGAGACTCCAAGGCCAAGCGAGGGCCTTGGAGCCAGGAGGGGCCCTGCTCTACTGGGG
GGACGACACCCCTTTCCCCTATCCCTCCCTGCTGGGGTGTGGCCTGATGTCTACGTGGCACCAGGCCCCTAAATCCTTTCAACATCCTGGCGGAGCG
GAGAGGCTGGGCCCTTTTGACAGGTGGGGAAACTGAGGAAGGTTGCAGGGAAGGCACTCATCCAAGGGTGCCTGGCCCCCCGCGGTGGGTGCCTCAC
TGGCTGGCCTAAACCTTTGCATCAAACCAGTTCCAGGATTGAACGCAACAGGCTGATGGGGACTGAGTCGTGGTACAGATGGCGGCAGTTGTGTGGC
CCTGGGCCCAGGAATGGCTGGGAAGCTCCCTTTCTTCTCTGGCTTGGGGGATGAGGTTAGTGTAAATTTCATGGAGTTGCCAGGAGGATTAAGGCCG
ACGTGCATTCACACGTGGCCCCGTGGCTGCCGCCAGTTAAGCCCTGCGCAGCCATTGGAAGTCTCATTGAGAAGGAGCCTCCCGGCTTCCTGCATTT
GTTCCAGGCGGGCTGGTAAGCGCCCTGCTTATTTGCAAGGCTGTGTGAGGACGAGCCCTGTAAACCCTAGGACGAGGCTGTGCTAATGTGGTTTCTG
TTCCACTGTCTCCCCTTCTCCTTTTGCCCCTCTTTCTGGGGAACCTGGAGCCCCTGATGCCCTCTGTTTTTTCCTGCTGCTGGGGTGGACCTAGAAC
CTCTCCCTCCATCCTTTCTGCTGCTGGGGTGGGCCTGCAGGAGGCTGTCCTCTTACCTGGTCCCTAACTTCCCTCCGATCAGCGGGGCTTCAGCGGA
GCCCCAGCGCATTGGACAGGTGGTGCTCTGTGGCCGGCACAGGCCAGGGCCCAGGGTGCGGCCCCTGCCTGTAGGTAGACATGCAGGCCCTTCTGAG
CAGAATGAGGGGGCGGTGAGGGCAGCTGTGCGGGTGTGGCTGGCCTGACGGCTCCCTGCAGCCCTGCGGCTTCTGTGCAGTGGGGGTGGGGCGGGCC
AGACCTTTGCAGGGCCTCTTGGCTGGTGGAGGGCTGAGTGAGCAGAGGCCCCAGCCCTCGTGTTCCCTGGGGTGTGGCCTTAGGATGGTCCCGGCAC
CCTGGGGACCCAGCCCCTGCTCACCTTATCCTCCTCCCGTGCTTGGCTGGGGTGTGGTGGCTGAGCCAGCCCACCCAGTGCGGTGGATGCTCGTCGG
AGGGCAGAGGGTGGGCTACCGGCTTAAGGGGCCCCAGGGAACCTGGGGGGTGGAGCCCAAGGGGTTCCAAGAAGGGGCGGGGCCAGGAACCCATAGA
CAAGAGGGTGGGGCAGGGAAAGGGCGTGGCAAGAGGGCCACCCACCTCCGGTCCAGAGAGCCAGCCAGACCCTTACTTTCTCTTGCTGTGTGACCTT
AGGCAAGGACATGCCACCACCGGCCTTAGTCTCCCTCTTTGTGAAGTAGGATGAAGATCCCCACCTGTGGAGCAGATGTGGGGCTGCATGGTTGGGT
CAGGATGGAACAGGATGGGGAGGCCGGGCGTGGTGGCTTACCCCTGTAATCCCAGCACTTTGGGAGGCAAAGGTGGGAGGACTGCTTGAGTCCTGGA
GTTTGGGCAACATAGCGAGACCACCCCCATCTCTACAAAATAATGTTAAAGTTAGCCAGGTGTAGTGGTGAGTGCCTGTGGTCCCACCTACTGGGGA
GACTGAGGCAAGAGGATCCTTTGAGCCCAGGAGGTGGAGGCTGTAGAGAGCCATGCTTGGGCCACTTGCACTCCAGCCTGGGCAACAGAGCAAGACC
CTATCTCTAAAAAAAAAATGGAATTGAGGATGTGTCCTCTGGGTGTGGGCTCTACCACCCACCAGCCTCCCTCTCCTGTGGGTGCTGCCTGCCACCC
ACCGCTGAGGTCCCTGCAGCATCAAGGTGCCCAAACGAAGACACTCTCCAGCTCTGAGCCAGGCACCCATACAGAGCCCGGCAGACGCCTCTGCTGC
TGCAGTTCTTAGAATCCAGAGCGCGTGGGGAATGTGAATTTGTGCTGCTGGAGCCAAACATCCCACCTCCAGGTTTTAGCTGGAGGAATCCATGTGG
ATATGCAAAGCAATGTAGTTATAATGAATAGCAATTGTGGGCTGGGCGCAGTGGCTCATACCTGTAATCCCAGCACTTTGGGAGGCTGAGGTGGGTG
GATCACCTGAGGTCAGGAGTTTGAGACCGGCCTGGCCAACATAATGAAACTCCGTCTCTACTAAAAATACAAAAAAATTAGCCGGGCATAGTGGCGG
GTGCCTGTAATCCCAGCTACTCAGGAGGCTGAGGCAGGAGAATCACTTGAACCCAGGAGGCAGAGGTTTCAGTGAGGCGAGATCGTGCCATTGCACT
CCAGCCCGGGAGACACAGCGAGACTCTCTTTCTCAAAAAAAGAATAGCAGCTTTGCTATAGCTTAGGGGAGCAAAACCCGAAAGCCACCTTAAGTTT
CCCGAAGATAGAGATCCCTGGGGTCCTATTCTGAGACAGAGTCTTGCTCTGTCGCCCAGGCTGGAGTGCAGTGGCGGATCTCAGCTCACTGCAGCCT
CCGCCTCCTGGGTTCAAGCAATTCTCCTGCGTCAGCCTCCCGAGCAGCTGGGATTACAGGCGCCCGGCACCACCCCCAGCTAATTTTTTTTTTTTTG
AGACAGAGTCTCGCATTGTCGCCCAGGCTGGAGTGCAGTGGCGCGATCTTGGCTCACTGCAAGCTCCACCTCCCGGGTTCACGCCATTCTCCTGCCT
CAGCCTCCCGAGTAGCAGGGACTACAGGCGCCCGCCACCGTGCCCGGCTAATTTTTTTTTTTTGTATTATTAATAGAGACGGGTTTCACCGTGTTAG
GATGATCTCGATCTCCTGACCTCGTGATCCGCCTGCCTCGGCCTCCCAAAGTGCTGGGATTACAGGCATGAGCCACCTTGCCTGGTCCTTTTTTTTG
TATTTTTAGTAGAGATGAGGTTTCACCATGTTGGCCAGGCTGGTCTCAAACTCCTGACCTCATGATCCGCCCGCCTCGGCCTCCCAAAGTGCTGGGA
TTACAGGTGTGGGCCACTGTGCCCGGCCAGTCCCATTCTACTTTTTAGGGACATGGAATCATGTAAAGATGCCATACAGAAAACAGTATGCAAATCC
AGAGACAGAGGACATCTGTGCATGTTTCTTTCTTTCTTCCTTTTTTTTTTTTTTTTTTTTTTTGAGATGGATTCTTGCTCTGTCGCCCAGGCTGGAG
TGCAGTGGTGCAATCTCAGCTCACTGCAACCTCTGCCTCCCAGGTTCAAGTGATTCTCCTGCCTCAGACTTCCAAGTAGCTGGGATTACAGGCATGC
CCCACCATGCCCAGCTAATTTTTGTATTTTTAGTAGAGGCGGGGTTTCACCATGTTGGCAAGGCTGGTCTTGAACTCATGACCTCAGGTGATCCACC
CGCCTCAGCTTCCCAAAGTGCTGGGATTACAGGCGTGAGCCGCCGTGGCAACCCGCATGCTTCTTATGTATAAGAAAAAAACAGCTAGAAAGTTGTG
GGTTCACTGGCTGGGCACATGGTGGCTTGTGTCTGTAACCTCAGCACTTTGGGAGGCTGAGTTAGGAGGATCATGTGAGGCCGGGAGTTCAAGACCA
TCCTAGGCAACATAGCCAGACCCTGTCTGTACCAAATAGAAAAAAAAATTAACTTGATGTGGTGGTGTGTGCCTGTAGTCTCAGCTATTTGGGAGGC
TGAGGTGGGAGGATCACTTGAGCCCAGGAAGTCGAGGCTACAGTGAGCTATGATTGTGCCACTGCACTCCAACCTGGGCAACAGAGCAAGACCCTGA
ATCAAAAAGAAAGAGATGTATTTGCCAACACAGGGGTGTTCTGCAGTGGGGTGGGAAGGGGACGTGTAGGACTTCATAGTTTCCTTTTTTTTGTTTT
AGAGACAGGGTTTAAAAAAAAATTTTTTTTTTTTTTTTGAGGCGGAATTTCATTCTTGTTGCCCAGGCTGGAGTGATCTCGGCTCACTGCCATCTCC
GCCTCCCAGGTTGAAGTGATTCTCCTGCCTCAGCCTCAGGCAATTTTGTATTTTTAGTAGAGACGGGGTCACTCCATGTTGGTCAGGCTGGTCTCAA
ACTCCCGACCTCGGGTGATCTGCCTGCCTTGGCCTCCCAAACTGCTGGGATTACAGGTGTGAGCCACTGTGCCTGGCCCCTTTTTTTTTTTTTTAAG
TAGGGTCTTGCTAAGTTGTCCAGGCTGGTCTTGAACTCCTGGTCTCAAATGATCCTCCTGACTTGGCCTCCCAAAAGGCTGGGATTGCAGGAGATAT
GAGCCACCGCACCCGGCCCTTTTATAAGTTTTCTAAAGGAAAGGAACATGTACCATCGTGTTTTATTTTAATTTTTACATAGAAGCAGGAACAATAG
CATGAACCCCCAGACACGGCCCCCCAAGATGCGCTGTTCCGCTGTGCTGCAGCCCCATTTGTCCTTTTCTATACCATTTTTGGTTGTATTTGAAGCA
AATCCCTGACATCAGGGCATTTCATCCCAAAATACTTCCATCTGTGTTTCTAAAAAAACGAGGCCATTTTCTTATGTAACCACAACGTGATCAACAG
TAATCAATGCTTAACATCTAATTCTGGGCCACGTTAAGACCCTCCTGATTGGCTCAAGAATGTCTTTTGTGCAGTTGGTTTCTTGATTCTTTTTGTT
TTTTTTGAGACAGGGTCTCACTGTGTCACCCAGGCTGGAGTGCAGTGGTGCAATCACGGCTCACTGCAGCCTCAGCCTCCTGCCTCAGCGATCCTCC
TGCCTCAGCCTCCCAAGTAGCTGGGACCACAGGTTTGCACCACCACTCCCAGCTAATTAAAAAAAGTAATTTGTAGAGACGGGGGTGGGGTGGGGGT
CTTGCTATGTTGCCCAGGCTGGTCTCAAACTCCTAGACTCAAACAATCCTCCTGCCTTGGCCTCCCAAAGTGTTGCGATCACAGGCATGAGCCCCTG
TCCCTGGCCCCTATTATTTCTCTAACTGGGGAAAGTCATGTGGGAACAGATGTAGCTTGCCTTGGCCTCTGACCGGCCCTGCCTGCGTTCCTGGGTG
CTCTCTGCTGCTTCTCATGTGTGCCACTGTGGGGACTCGGCCGCCCACCCCACCCCGTGGTGTTACCTTGCGTGTGTAGTTCTGTGAGCTCAGGGCT
ATGGTCTGCCAGAACTAGGGGGCGTGGGGCCCCAGTACCAGCCCAAGGCCTCCTCTCTGCAGGTATGGACCTGTCTGCCAATCAGGACGAGGAGACC
GACCAGGAGACCTTCCAGCTGGAGATCGACCGCGACACCAAAAAGTGTGCCTTCCGTACCCACACGGGCAAGTACTGGACGCTGACGGCCACCGGGG
GCGTGCAGTCCACCGCCTCCAGCAAGTGAGTGCCTCGCTCCCACCTGTCACCGCCCCCACCACCTTGCCTGGGCTACCCCGCCTGACCCTGTCCCGC
CATCCCCCAGGAATGCCAGCTGCTACTTTGACATCGAGTGGCGTGACCGGCGCATCACACTGAGGGCGTCCAATGGCAAGTTTGTGACCTCCAAGAA
GAATGGGCAGCTGGCCGCCTCGGTGGAGACAGCAGGTAACACTAAAGCCCCAGTTCCCTGGAGCCGTCCTGGAGTCCTGGAGGGTCTGGCCATGCCG
TGGTCACTTGGTAGCCCCAGCCAAGGCCTGCTCTGTGCTGGGCATCCCCCCGGACTGGCCCCGCACTGTCCTACCCTGGGGCTGACTGCTGTGTGAC
CCCAGCTCCTGGCCCTCCCTCTCTGGTCACCCCAGCCTCCACCCCACTCCCTGCCAGGAGGCTCACTGACTCCCCTCTTTCTGGGACAGGGGACTCA
GAGCTCTTCCTCATGAAGCTCATCAACCGCCCCATCATCGTGTTCCGCGGGGAGCATGGCTTCATCGGCTGCCGCAAGGTCACGGGCACCCTGGACG
CCAACCGCTCCAGCTATGACGTCTTCCAGCTGGAGTTCAACGATGGCGCCTACAACATCAAAGGCAGGTTCTCCTGTGGGCAGCTGCTGGGCAGGGA
ACCCCTCGGTCGGGGCTGGGGTCAGTGCTGCGGGGAGCGCCCTCTGCATCCACACTGGACCCTGGCTTGGCTCAGGGCCATTCCAGGCCCTAAAGGG
ACAGGTGTCTGATGGCCACCAGGGGCTCTGGGATGCAAGCAGCCCCTTTCCCTCTTGTCTGTGTGGTTGGGGGGACTTACCTTGCCCACCTGACAGA
GAGGTGTGTGGAGGGGAGAGCAGGGAGGGGAAGGAGAGCAGGGAAGGGGAGGGAGGAGAGCAGGGGAGGGGAGAGCCGGGAAGGAGAGGAGAGCAGG
GGTGGGGAGGTTCTGGAAAGGGTGTGCAGGGGAGGACGCGCCTCGGTTATGGGACTGGAGCCCCTTCCCAGGAGGACCCCCAACAATCCAGAGGTGC
CTGTTAGGATTCAGAACATGGTTTTTTTGTTTGTTTTTTTGAGACTCACTCCCTCACCCTGGCTGGACTGCAGTGGCGTTATCTCGGCTCACTGCAA
CCTCTGCCTCCTGGGTTCAAGTGATTCTCCTGCCTCAGCCTCCCAAGTAGCTGGGATTACAGGTGTGCACCACCACGCCTGGCTAATTTTTATATTT
TTAAAATTTATTATTTATTTATTTTGAGACCCAGTCTGGAGTGCAGTGGCGTTATCTCGGCTCTCTGCAACCTCTGCCTCCTGGGTTCAAGCGATTC
TCCTGCCTCAGCCTCCCGAGTAGCTGGGACTATGTGTGGGAGCCACCATGCCTGGCTAATTTTTTTGTATTTTTCATAGAGACGGGTTTCACCATGT
TGTCCAGGCTGGTCTTGAATTCGTGGCCTCAAGTGATCCGCCCACCTCAGCCTCCCACAGTGCTGGGTTTATAGGTGTGAGCCACCACACCCGGCTA
ATTGTTTTGTATTTTTAGTAGAGACGGAGCTTCACTATGTTGGCAAGGCTGGCTCGAACTCCTGACCTCAAGTGATCCGCCCACCTCAGCCTCCCAA
AGTGCTGGGATTACAGGCGTGAGCCACTGCGGCCGAGCAGAACACGTTCTAGGACCCTTGTTCATGTGTCCATCATGGACAGGAGGACGTGCGGGCC
ATAGGGACCCTGGCTCATTCCGGAGCCGGGACTGGAGGGTGGGGCGTCACCCTTGGGAACACCCGTGCCCACCCTCCGCTGCCCAGGGTAGGGGTGG
GGAGCCAGGCTTTGGGCCCCACTTGATAAAGTCCCCTCCCCAGACTCCACAGGCAAATACTGGACGGTGGGCAGTGACTCCGCGGTCACCAGCAGCG
GCGACACTCCTGTGGACTTCTTCTTCGAGTTCTGCGACTATAACAAGGTGGCCATCAAGGTGGGCGGGCGCTACCTGAAGGGCGACCACGCAGGCGT
CCTGAAGGCCTCGGCGGAAACCGTGGACCCCGCCTCGCTCTGGGAGTACTAGGGCCGGCCCGTCCTTCCCCGCCCCTGCCCACATGGCGGCTCCTGC
CAACCCTCCCTGCTAACCCCTTCTCCGCCAGGTGGGCTCCAGGGCGGGAGGCAAGCCCCCTTGCCTTTCAAACTGGAAACCCCAGAGAAAACGGTGC
CCCCACCTGTCGCCCCTATGGACTCCCCACTCTCCCCTCCGCCCGGGTTCCCTACTCCCCTCGGGTCAGCGGCTGCGGCCTGGCCCTGGGAGGGATT
TCAGATGCCCCTGCCCTCTTGTCTGCCACGGGGCGAGTCTGGCACCTCTTTCTTCTGACCTCAGACGGCTCTGAGCCTTATTTCTCTGGAAGCGGCT
AAGGGACGGTTGGGGGCTGGGAGCCCTGGGCGTGTAGTGTAACTGGAATCTTTTGCCTCTCCCAGCCACCTCCTCCCAGCCCCCCAGGAGAGCTGGG
CACATGTCCCAAGCCTGTCAGTGGCCCTCCCTGGTGCACTGTCCCCGAAACCCCTGCTTGGGAAGGGAAGCTGTCGGGTGGGCTAGGACTGACCCTT
GTGGTGTTTTTTTGGGTGGTGGCTGGAAACAGCCCCTCTCCCACGTGGCAGAGGCTCAGCCTGGCTCCCTTCCCTGGAGCGGCAGGGCGTGACGGCC
ACAGGGTCTGCCCGCTGCACGTTCTGCCAAGGTGGTGGTGGCGGGCGGGTAGGGGTGTGGGGGCCGTCTTCCTCCTGTCTCTTTCCTTTCACCCTAG
CCTGACTGGAAGCAGAAAATGACCAAATCAGTATTTTTTTTAATGAAATATTATTGCTGGAGGCGTCCCAGGCAAGCCTGGCTGTAGTAGCGAGTGA
TCTGGCGGGGGGCGTCTCAGCACCCTCCCCAGGGGGTGCATCTCAGCCCCCTCTTTCCGTCCTTCCCGTCCAGCCCCAGCCCTGGGCCTGGGCTGCC
GACACCTGGGCCAGAGCCCCTGCTGTGATTGGTGCTCCCTGGGCCTCCCGGGTGGATGAAGCCAGGCGTCGCCCCCTCCGGGAGCCCTGGGGTGAGC
CGCCGGGGCCCCCCTGCTGCCAGCCTCCCCCGTCCCCAACATGCATCTCACTCTGGGTGTCTTGGTCTTTTATTTTTTGTAAGTGTCATTTGTATAA
CTCTAAACGCCCATGATAGTAGCTTCAAACTGGAAATAGCGAAATAAAATAACTCAGTCTGCAGCCCCAGGCCGGCCTGTGTGTGTCTTGGGGCTGA
GGTGGGTGGGGGGGCTGAGGTGGGTGGGAGGGCTGGCGGGACAGGTAGGCGCCCTGGCTCCCCAGCTCAGTGCTGGGAGTGTGCAGTGGGAGGGAGG
CCGTGGCTCCAGTGGGTGCTCCGGAGCTCGTGGGCCCAGCACACCTCCTTAAGCGGGGGATGGAGCGCTGGGAGGGGGTGGACTGTGGCCCATGCGA
CCCCCAGAGCCATTAGGAGGAGTTCTGTGGTGAGAAGTGGCTGTGGCTCCTCGTAGGCTACGTCCACCATGCGGGGGACCTCGGGGGTGTCTGGCGG
TGGCACGCTGGATGTTGAGAAGGCGCAGCCCAGGGAACACTCAAACCAGGAGACCCCACATTATCCTCTAGTTGACATGTGCCCTTCGACTAGGGGA
CTTGGTGGTAGGGGCAGCTCCGCCCCCATACTGCGAGGATCCGGGCCTTCCACTTCCCCAGACACAGCAGGTTGGGGTGCCCCTGGGGCTGGGAGAT
GCTGCCCTGGGCCCCTCAGGGGGCGGCCGTGGTAGGATGGACCGGGCCGAGCGTGCAGACCCTGGGCAGGTCCCTACATCGGAACCAGCAGCCCTGG
GGATATCTGGCATATCAGAGGCTGAGGACTGTTTCCAGACTTTACCAAGGCCACAGTCAGGGCGACCTGGGTTCTGAGCCTCCCTTTGCATCCTAGG
CTGCAGGGACAGGGGCTGGGCAGAGGCTCGCGGACCTCGTGCAGCTTCATTCACTCCCAGGTGCCCCTAAGGATAGCAGCTGCGAGAAGACTGAGGG
GAGGAGCAGTAAGGCGCAGCCTGGAGCGGAGGGACCTGGCGTCCAGTCATTGTGCGCTGGGAAGGCGGTGATGGCGACGGAATTTGGGACACGCGGG
GCGTCCCGGGGGCAGCCAGGGCACTGCAGGCCGGAGCCCCCTGTTCCCCCGCATCCTCCCCGCCGTCGGCAGCAGAGCAAGCGCTGATTGGCTGCTG
ATGACGCAACTGGGAGGGCTGCGCTGTGATAGGTGGTCCCTCGGGGGCGTGGGGCGCAAGTCTTGAGATTGGCAGGGGCAGGTGGCTGCTGCAGAGG
GAAGTCGGGGTCACCTCAGCGAGGTTCCGGCGGCCACCCCTCTCCCCTCCCCCATGAAAGCCAGGCTGGAAGCCAGAAAAATCCCAGTGACTGGAAG
GGACAGATTAATCCTCCGGAACCAAACTCTTTGAGACCTGGGGAGGAGGGTGGGAGGCACTGGGAAGGGGGATTGGGGGGAGCCTGGCTCATTTCCA
CCCATGGAGCTGACCTAGAATGACCCTAAGGTCCCCGGAGCCACCAGCTGATTCCGAATTCCATTCATTCTGCAAAATTTGGCGCCTACCACGTGGC
CGAGATTCCCGAGGCTGCAGCCAATGAGCAGCATAGTCCGATGGGGGAGGCTGGAAGGGCCGTTCACGAGATCTCTCGGGAGGAATCGGTAGGAGCT
GCCCAACTGAAAGGGACGGGGGAATAACCCGGGGCGAGGGACCTGCCCAGGCCCTGCCCTCTGGGGGGAAGCCAGGCCAGGGGCCGGGGGCATCTCT
GGGGTCCCAGGTGAGATGCGGGTCGAGGGAGCCAGGCCACGGTTCTCCAGACTCGCGTGGAGGCCACGAGCGTCTTCTGGAGGAGCGGGCACTGCGC
GGACCGGCAGACTCTGGGGCGCTCGTGCTCCCCATCTCCTGACCTTTTCCTACCTTCAGTTCCCTCGTGTCAGGACAGGTTCTGGGGCTCGCCGGGG
CAGGGAGCCAGGGCAGAGGGTGCAGTTTCCGCTCCTGCCTGAGTCACCTTCGCTTTCTCAGCGATTCTTCATTCACAAGGGCGTGGCCCATCACACG
CACTCACACACTTGTGAACTTGCACGCACACACACATATGGACCTGTGCAAAGAGCCCCATGCACAGGCAGGTACACGTGCATGTAGGCGTGCACAC
AGACACGCACAGATGCAGGCAGACCAACTCCCAGGAATCTGTCTCCAGCCTACAGGGATTCAGCACTCAGGGTACCAGCCTCTGTCCCGTGCACCCC
CATTCCCGGCAGTTCTCCCAGGTGGGTCGGGGGCCACTAGGGACCCCATACCCCGAGGGTGGCTCCTCCATTAAGCCTGCCATGGGGTTGGGAGTGA
GAGTCCCACCTCCCACTTGAGAGTCCCCAGTGAGTCAAGGCAAGTGCAGCCAGGGCTCCAAGATCCTTGTTGGGATTCACTGTGATCCCCAAAGATC
ACGACTTAAGGGAAAACATTCAATTAAACTTGTTAGAGCAAAGAAAGGAGAGACTGGGCTGGGAGCAGTGGCTAACGCCTATATAGTTCAGCTACTC
AGGAGGCCCAGGTGTGAGGATTGCTTGAGTCCAGGAGGCTGAGGCTGCAGTGAGCTATGATGGCACCACCCACTGCACTCCAGCCTGGGTGACAAAG
CAAGACCCTGTCTCAAAAAAAAAAAAAAAAAAAAAAAAAAAGGAAAAAGAGGCCGGGCACAGTGGCTCACACCTGTAATCTACTAAAAATAAGAAAA
TTAGCCAGACATGGTGGTGGGTGCCTGTAATCTCAGCTACTTGGGAGGCTGGGGCAGGAGAATCGCTTGAACCCAGGAGGCGGAGGTTGTGTGAGCC
GAGATTACACCACTGCACTCCAGCCTGGGCGACAGAGTGAGACTCTGTCTCCAAATAATAATAATAATAATAATAAAATAAAAAGAAAAAAGAAAGA
AAGAAAGGAGAGACTGAGCTATATTGCCCAGTCCAGGCATGTGGGCAGTCTGGGGGACAGTGGGGCATACTCGGTGTCTCAGGGTCCCAGCCCTGGG
TGCCCCCACTGCTGTCTGCTGTGACCTCCTCAGGAGGCCTCATTGGGAGGGACCCTGAGTCCTGCGGGGGGCTGTGGAGCTGTCACCAGCCCCAGGA
GGACTCAGGCAAGTCCCTCTGGTGGTCGAGGCCTCTGAGCTTCCCAGTGTGTCTCCACGGGACACCCCCCCTCCAGACCTTCTGCCCCAGCTCAGCC
TCTTCCAATTCATTATCAGGGGGTGGGGGAGGGGACAGGAAGCCCCAGACCCAGCTGGGCCCCATTTCCTCCAGGGCCACAGTGGCCTCTCCCAGAG
TCACGAGCATGGGGGCTGATCTCACTCTTTTTTTTTTTTGAGACGGACTCTCACTCTGTCGCTTTTTTTTTTTTTTTTTTTTTTGAGACGGAGTCTC
ACTCTGTCGCCCAGGCTGGAGCGCAGTGGCGTGATCTCGGCTCACTGCAATCTCCACCTCCCACGTTCAAGCGATTCTCCTGCCTCAGCCTCCTGAG
TAGCTGGGACTACAGGTACGTGCCACCACACCTAACTAATTTTTGTATTTTTTAGTAGAGATGGGGCTTCACCATATTAGCCTCCCAAAGTGCTGGG
ATTACAGGCATGAGCCACCATGCCCGGCCTTGACTTCACTCTTTTAAAAAAGTCAAATACTTGGTGCTTGAAATCCCAGCACTTTGGGAGGCTGAGG
CAGGAGAACTGCTTGAGCTCAGGAGTTTGAGACCAGCCTGGGCAACATGGTGAGACTCCTGTCTCTGTAAAAACTACGAAAATTAGTCAGGCATGGT
GGTGCGTGTCTGTCGTCCCAGCTGCTCAGGAGGCTGAGGTGGGAGGATGGTTTGATCCTGGAAGGTTGAGACTGCAGTGAGCTGTGATTCCACCACT
GCAGTCCAGCCTGGGCCGCAGAGCAAGACTCTCTCTCAAAAATAAATAAAATAAAATAAAAAGTCTAAAACTTTTCTTTCAAACGACGGCTTTTGGG
GCTCATAAGGTGCGTGAAGATGGAAAGGGGGCCCGGCAGTGCCCATTTCGGCTCCCTTGGGACCCCGGCTTCCCTGAAGACCCCTGAGCAATGGTTG
GCTGGGTGCCTGGTCTCCCTGAACGGGCTGTGGGCAACAAGGTTGTCTGGATGGGGGAGGGGCTCTGAGACCCTTGGGGGCAGCAGAGGGAGAAGGG
AACAGATGGGGTCTCCCAGGCAGAGAGGCCTGGAGGGGTGGGGCGGGGCAGGGCATTTGGCGTGAAACCCAAGCCCCTTAACGGGGCCCTAGCTTAA
ACCGGGAGGAGGGGCCTCTGTGATGTGCAAATAATTAACACAGAAGCCTTCACTGTGCTCCCCTCCTCATCCCAGGACCCAGGTCTGACCCTCAGGA
GGGGAGGAGGTGAGCAAGGGGGGAACCCACCTTCCAGGGAACCCCCAGATAGTGCCAGGCACCAGGCAAGTGCTCTAAAAGGCTTTACTAATATTAT
TTGAAGAAACTAAGCTAATAAATAAATACGGTATAAAATCAAAGGGTACCCACAGATATAAACAAAAATCCACAGCTAACCCGGCCCCAGCCCTCTT
TCCTTTGGAGGTAACTGTGTTAATGGTTTTTTACACCCCTCTGGAATTTTTTTTTTTTTTTTTTTTTTTTTTTTTTTTTTGGGAAGGAGTCTGGCTC
TGTCGCCCATGCAGCGCAGTAGCGTGATCTCGGCTCACTGCAACTTCCACATCCCGGGTTAAAACGATTCTCCTGCCTCAACCGCCTGAGTAGCTGG
GACTACAGGGGCGCACCACCATGCCCGGCTAATTTTTTTTTTTTTTTTTTTTTTTTTTGAGCTGGAGTCTTGCTCTGTCGCCCAGGCGGGAGTGCAG
TGGCGCAATCTCGGCTCACTTGCAAGCTCTGCCTCCCGGGTTCACGCCATTCTCCTGCCTCAGCCTCCTGAGTGGGTGGGACTACAGGCTCCAGCCA
CCACGCCCGGCTAATTTTTTGTATTTATTTTTTAATAGAGATGGGGTTTCACTGTGTTCGCCAGGATGGTCTCGATCTCCCTTTTTTTTTTTTTTTT
TTTAGACGGAGTCTCGCTCTGTCGCCCAGGCTGGAGTGCAGTGGCATGATCGTGACTCACTGCAAACTCCACCTCCCGGGTTCACGCCATTCTCCCG
CCTCAGCCTCCCGAGTAGCTGGGACTACAGGCGCCCACCACCACACCCGGCTATTTTTTTGTATTTTTAGTAGAGACAGGGGTTTCACCATGTTAGC
CAGGATGGTCTCGATCTCCTGACCTTGTGATCCTCCTGCCTCGGCCTCCCGAAGTGCTGGGATTACAGGCGTGAGCCACCGCGCCCGGCCTCTTGTT
TTTTTTTTTTTTTTTTTTTATAGACTTCTAAGGAAAGCAACACTAAGAAAATATATTTTTAAGAAATTATTCAGCCAGGCGTGGTGGCTCACGCCTG
TAATCCCAACACTTTGGGAGGCTGAGGCGGGTGGATCACTTGAGGCCAGGAGTTTGAGACCAGCCTGGCTCACATGGTGAAACCCTGACTCTACTAA
AAATACAAAAAATTAGCCGGGCACAGTGGCATGCTCCTGCAATGCCAGCTACTTGGGAGGCTGAGGCAGGAGAATCCCTTGAACTGGGGAGGCAGAG
GTTTCAGTGAACCAAGATCGAGCCACTGCACTCCAGCGTGGGTGACAGACTGAAACTGTGTCTCAAAAAAAAATTTTTGTTTTTGGAGATGGGGTCT
TGCTCTGTCACCCAGGCTGGAGTACAATGGGGTGATCTAGGCTCACTGCAACCTCAAACTCCTGGGCTCAGGTGATCTTCCTGCCTCAGCCCCTGGA
GTAACTGGCACTACAGGTGCATGTCAGAACACCTGGCTAGTTTTTAACTTTTTTGTGTGTAGACAGGGTCTTGATATGTTGCCCAGGCTGGTCTCCA
TCACCTTCTGGGCTCAGTGATCCTCCTGCTTCAGCCTCCCAAAATGCTGGGATTACAGGTGTGAGCCACCATGCCCGACCCTGAGGGATACCTTAAT
GTTTATTTCTCACATTTCTTCTAGAACAAACATTGCCAGCGCATCCCAGACTCTGAACTGTGGCTGGGGACTGCAGCATGGAGGTCTTTGCAGGCAC
TCTGGGAAACATGCCACAAGCAGGGTATTAACCCAAGACAGTCAGTCCCTCCAATGAGAAAAAGGAGCCGAGGGACTGTGCATGCCTCAGAGCCAGA
TGCAGCAGAACTGGTGGGTTGAGGGGGCACTGAGCGCCTGGTGCCTCCTGCTGCCCCCCACCAGCTCCGACAGTCCTCGTCCTTGTCCTCCCCATCA
GAAGGGAAGAGTGAATTGTCTGTGGGATGTGCCAGTGTCCCCGGTTCTGAGGTTGGGCAGGCTGGGGAGCCCCAGACATGGCTTGCTGCCCACTGTG
GAGCCTCCTGGACTTAGTTCCAGGCTCTGCCCGATCCTCTGAGGGTGTGGCTTCAAAGCCCTGCTTGGGCCTAAGGCCATCCCAGGGCAGTGACTGA
GGGTAGACTGAGATGTCCCTTGGGCACCAGTCCTAGGGTACCTGGGCTGGGGGTCTTCAGCTCAGGCCCCCTAGTGCCCGTCCTTTGGGCTGTGGGC
AGGTTTCCACAGCACTGGCCCTTTCTGACCTTACACCCCTGTGCATCCTCACTGGGGCTCTGTGAAGGTGAAAGTCACACTAAGGACTGTGCCTATG
GTGGCCAGGGCACCCCACCGGCAGCACACCTCAGAGGCCTCATCCTAGGCGCTGCTCACTTTTTTTTCTTTTTTTTCCTTTTTTTTTTTTTTTTTTT
TTCCGAGACAGAGTCTTGCTCTGTCACCTAGGCTGGAGTGCAGTGGCATGATCTCGGCTCACTGCGACCTCCTCCTCCTCCTGGGTTTAAGCAATTC
TCTTGCCTCAGCCTCCTGAGTAGCTGGCATTACAGGTGCCCGCCACCACGCCCGGCTATTTTTGTAGTTTTAGTAGAGACGGGGTTTCACCATGTTG
GCCAGGCTGGTCTCAAACTCCTGACCTTGTGCTCTGCCCGCCTTGGCCTCCCAAACTGCTGGGATTACAGGCGTGAGCCACTGTGCGTGGCCCTGCT
CATTTTCTTACCACAGCTTTCCAGAAATAAAGTTAGGATGGAAAGAGAGAGAGAGAGATTGAGAGAGAGAGAGAGCACCACATTCAGATAGAGACAG
GAGAGTTCTCAGCAAGAAGAGAACTCAGCTCAGAGTTGAGAATGTCTCAACTCAGCTGTCTCCCTGCTGAGACGTCCTGGGCTGAAGTGTCCCCTCA
CCCCTGCGGGGCTGTGACTGCACACTGCACACATGGGTCACAGCAGGGCTTGGCGTTTGGAGCAGGCCGGGTTGGTGGCTTGTCCTCATCTCTTGCC
TGGAGCTGTGCTGGCCTGTGGTTTGCTTTGGCTGCTACCATGTGGAGGGAGAGGTGCTTGTGTCCATGCCTGGCCTCTTGGAAGGCCACCACTGTGA
TGGAGAGAGGAAGGCCACCACTGTGATGGAGAGAAGCCGGGGCCAGACCGATGAGGGCTGAGAGCCCATGGGCAGGGGTTGTGGTCTCGGCCAAACC
CCAGCTGGCAGCTGATGGCAGCCACCCCGGGGTCCTCAGATATGGCACAGCTGCCTGGCTGAGCCTCGGCAAAGGGAAGCCTTGTGGAAAATAGCAA
CTCAGGGATGTTGTGAGTAGTGGGCTGAGCTGTGGTTTATTTTTTAGAGACAGGGTCTTGCTCTGTCACCCAGGCTAGAGTCCAGTGGTGTAATCAT
AGCTCACTGCAGCCTTGACCCCCCAGGCTCAAGTGATCCTTGTGTCTTGGCCTCCTGAGTAGCTAGGACTATAGGTGCATACCACCATGCCTCGCTT
GTTTTTTGTTTTGATAGAGATGGGGGTCTTGCTAGGTTGCCCAGGCTGGTCTGTAACTCCTGGGCTCAAGTGATCCTCCTGCTTTGGCCTCCCAAAG
TGCTGGGATTACAGGCGTGAGCCACTGTGCTGGCCTTGGGGTGTGGTTTTATGTGGCCATAGATGACTGAGATGGGTGAGAAGCGGGACCTAGGGGA
ATATACAGGGCAGGGGGATCCGACAGGGGGACAGGAGGGCAGACAGCTCCTGGAAGGCCTGGGAAAGAGATATCAGGGCTGGACCCTGGAGAAGTGA
GGCTTTGAGGGAGGGGAGGAGATGGGGCCTGGCGGGGTACGGGCGCTGCTCCTAGGTCTGAGGGCATCGGCGGTCTGAGAGCTTGCGGGAGGGAGGG
CTGCCTCCCGGTGCTCGGTGCCCTGGCTCTCCTCCTTGCTGCTGGCTGGCCTCCTCCTCCTCATGCATCTGTGTGTCCTGGAACACAGGGCACTGTC
CCTCTGCCTGTCCAGAAAAGCACGTGCTCCTTGGGGCCTGGCCTGCTCAGCCCTATGTCCCACCCGCTCCCTGCTGCTATTCCCAGGCCTTCGGGCC
TCACTGCCTGGGGTGGCCGTGCTCAGCCCCACCCTGTGGTGCCATGGCCTCCCCACCCCTGCCCCGAACTGCCCCTGGGACCAGTTCCTTCAGCTCC
CAGGACAGGGCCTGGCCATCCCTGTGGATTCTCCCTGTTCGTGGACCCCCATTGGTGCCCAGTCTCCTTGGACTGTCTTCTGAGCCTCTCTCAGATG
TACCCCCAACTGTACCCTTTCACCCAGAGGCCATTTGGCAGCGTCCTCCAGGACTCCTGCCGTGGGGGAAGTCTCCACCAAGGCCAACGCGTGACTG
CATCCCGAGGAGCTGATTTAAGATCTGGTTCTCAGGAACACCTGGAACTGGCCAGTCAGCATCCCCGGCCCTGCGGTCTGCACTTGGACAAGGGTCA
GGCAATGAGGATGAGCAGCAGGCGGGGCTTCTCCTGGAAGCCTTCCCAGCCTCCTGGGCCACTCCAGTGGGTCCCCTCCACAGACTCAGCCTGTTCC
CTGCCACCTGGTGCCGGTTTCTCEGTGGCACCTACTAGGGGCCGAGTGCCCCGGGACTGCCCAGCTGAGCAGCGCCGTCCAGGTCTGCACCCAGCTA
CACTCAGGGCACGTGCACAGGTGAGCAGCCGGGCACGCAGGCACCTGCCAGGTGTGGGTGTGGACTAGGTGCTCCCGG
HUMAN SEQUENCE - mRNA
GCGGAGGGTGCGTGCGGGCCGCGGCAGCCGAACAAAGGAGCAGGGGCGCCGCCGCAGGGACCCGCCACCCACCTCCCGGGGCCGCGCAGCGGCCTCT
CGTCTACTGCCACCATGACCGCCAACGGCACAGCCGAGGCGGTGCAGATCCAGTTCGGCCTCATCAACTGCGGCAACAAGTACCTGACGGCCGAGGC
GTTCGGGTTCAAGGTGAACGCGTCCGCCAGCAGCCTGAAGAAGAAGCAGATCTGGACGCTGGAGCAGCCCCCTGACGAGGCGGGCAGCGCGGCCGTG
TGCCTGCGCAGCCACCTGGGCCGCTACCEGGCGGCGGACAAGGACGGCAACGTGACCTGCGAGCGCGAGGTGCCCGGTCCCGACTGCCGTTTCCTCA
TCGTGGCGCACGACGACGGTCGCTGGTCGCTGCAGTCCGAGGCGCACCGGCGCTACTTCGGCGGCACCGAGGACCGCCTGTCCTGCTTCGCGCAGAC
GGTGTCCCCCGCCGAGAAGTGGAGCGTGCACATCGCCATGCACCCTCAGGTCAACATCTACAGTGTCACCCGTAAGCGCTACGCGCACCTGAGCGCG
CGGCCGGCCGACGAGATCGCCGTGGACCGCGACGTGCCCTGGGGCGTCGACTCGCTCATCACCCTCGCCTTCCAGGACCAGCGCTACAGCGTGCAGA
CCGCCGACCACCGCTTCCTGCGCCACGACGGGCGCCTGGTGGCGCGCCCCGAGCCGGCCACTGGCTACACGCTGGAGTTCCGCTCCGGCAAGGTGGC
CTTCCGCGACTGCGAGGGCCGTTACCTGGCGCCGTCGGGGCCCAGCGGCACGCTCAAGGCGGGCAAGGCCACCAAGGTGGGCAAGGACGAGCTCTTT
GCTCTGGAGCAGAGCTGCGCCCAGGTCGTGCTGCAGGCGGCCAACGAGAGGAACGTGTCCACGCGCCAGGGTATGGACCTGTCTGCCAATCAGGACG
AGGAGACCGACCAGGAGACCTTCCAGCTGGAGATCGACCGCGACACCAAAAAGTGTGCCTTCCGTACCCACACGGGCAAGTACTGGACGCTGACGGC
CACCGGGGGCGTGCAGTCCACCGCCTCCAGCAAGAATGCCAGCTGCTACTTTGACATCGAGTGGCGTGACCGGCGCATCACACTGAGGGCGTCCAAT
GGCAAGTTTGTGACCTCCAAGAAGAATGGGCAGCTGGCCGCCTCGGTGGAGACAGCAGGGGACTCAGAGCTCTTCCTCATGAAGCTCATCAACCGCC
CCATCATCGTGTTCCGCGGGGAGCATGGCTTCATCGGCTGCCGCAAGGTCACGGGCACCCTGGACGCCAACCGCTCCAGCTATGACGTCTTCCAGCT
GGAGTTCAACGATGGCGCCTACAACATCAAAGACTCCACAGGCAAATACTGGACGGTGGGCAGTGACTCCGCGGTCACCAGCAGCGGCGACACTCCT
GTGGACTTCTTCTTCGAGTTCTGCGACTATAACAAGGTGGCCATCAAGGTGGGCGGGCGCTACCTGAAGGGCGACCACGCAGGCGTCCTGAAGGCCT
CGGCGGAAACCGTGGACCCCGCCTCGCTCTGGGAGTACTAGGGCCGGCCCGTCCTTCCCCGCCCCTGCCCACATGGCGGCTCCTGCCAACCCTCCCT
GCTAACCCCTTCTCCGCCAGGTGGGCTCCAGGGCGGGAGGCAACCCCCCTTGCCTTTCAAACTGGAAACCCCAGAGAAAACGGTGCCCCCACCTGTC
GCCCCTATGGACTCCCCACTCTCCCCTCCGCCCGGGTTCCCTACTCCCCTCGGGTCAGCGGCTGCGGCCTGGCCCTGGGAGGGATTTCAGATGCCCC
TGCCCTCTTGTCTGCCACGGGGCGAGTCTGGCACCTCTTTCTTCTGACCTCAGACGGCTCTGAGCCTTATTTCTCTGGAAGCGGCTAAGGGACGGTT
GGGGGCTGGGAGCCCTGGGCGTGTAGTGTAACTGGAATCTTTTGCCTCTCCCAGCCACCTCCTCCCAGCCCCCCAGGAGAGCTGGGCACATGTCCCA
AGCCTGTCAGTGGCCCTCCCTGGTGCACTGTCCCCGAAACCCCTGCTTGGGAAGGGAAGCTGTCGGGAGGGCTAGGACTGACCCTTGTGGTGTTTTT
TTGGGTGGTGGCTGGAAACAGCCCCTCTCCCACGTGGGAGAGGCTCAGCCTGGCTCCCTTCCCTGGAGCGGCAGGGCGTGACGGCCACAGGGTCTGC
CCGCTGCACGTTCTGCCAAGGTGGTGGTGGCGGGCGGGTAGGGGTGTGGGGGCCGTCTTCCTCCTGTCTCTTTCCTTTCACCCTAGCCTGACTGGAA
GCAGAAAATGACCAAATCAGTATTTTTTTTAATGAAATATTATTGCTGGAGGCGTCCCAGGCAAGCCTGGCTGTAGTAGCGAGTGATCTGGCGGGGG
GCGTCTCAGCACCCTCCCCAGGGGGTGCATCTCAGCCCCCTCTTTCCGTCCTTCCCGTCCAGCCCCAGCCCTGGGCCTGGGCTGCCGACACCTGGGC
CAGAGCCCCTGCTGTGATTGGTGCTCCCTGGGCCTCCCGGGTGGATGAAGCCAGGCGTCGCCCCCTCCGGGAGCCCTGGGGTGAGCCGCCGGGGCCC
CCCTGCTGCCAGCCTCCCCCGTCCCCAACATGCATCTCACTCTGGGTGTCTTGGTCTTTTATTTTTTGTAAGTGTCATTTGTATAACTCTAAACGCC
CATGATAGTAGCTTCAAACTGGAAATAGCGAAATAAAATAACTCAGTCTGC
HUMAN SEQUENCE - CODING
ATGACCGCCAACGGCACAGCCGAGGCGGTGCAGATCCAGTTCGGCCTCATCAACTGCGGCAACAAGTACCTGACGGCCGAGGCGTTCGGGTTCAAGG
TGAACGCGTCCGCCAGCAGCCTGAAGAAGAAGCAGATCTGGACGCTGGAGCAGCCCCCTGACGAGGCGGGCAGCGCGGCCGTGTGCCTGCGCAGCCA
CCTGGGCCGCTACCTGGCGGCGGACAAGGACGGCAACGTGACCTGCGAGCGCGAGGTGCCCGGTCCCGACTGCCGTTTCCTCATCGTGGCGCACGAC
GACGGTCGCTGGTCGCTGCAGTCCGAGGCGCACCGGCGCTACTTCGGCGGCACCGAGGACCGCCTGTCCTGCTTCGCGCAGACGGTGTCCCCCGCCG
AGAAGTGGAGCGTGCACATCGCCATGCACCCTCAGGTCAACATCTACAGTGTCACCCGTAAGCGCTACGCGCACCTGAGCGCGCGGCCGGCCGACGA
GATCGCCGTGGACCGCGACGTGCCCTGGGGCGTCGACTCGCTCATCACCCTCGCCTTCCAGGACCAGCGCTACAGCGTGCAGACCGCCGACCACCGC
TTCCTGCGCCACGACGGGCGCCTGGTGGCGCGCCCCGAGCCGGCCACTGGCTACACGCTGGAGTTCCGCTCCGGCAAGGTGGCCTTCCGCGACTGCG
AGGGCCGTTACCTGGCGCCGTCGGGGCCCAGCGGCACGCTCAAGGCGGGCAAGGCCACCAAGGTGGGCAAGGACGAGCTCTTTGCTCTGGAGCAGAG
CTGCGCCCAGGTCGTGCTGCAGGCGGCCAACGAGAGGAACGTGTCCACGCGCCAGGGTATGGACCTGTCTGCCAATCAGGACGAGGAGACCGACCAG
GAGACCTTCCAGCTGGAGATCGACCGCGACACCAAAAAGTGTGCCTTCCGTACCCACACGGGCAAGTACTGGACGCTGACGGCCACCGGGGGCGTGC
AGTCCACCGCCTCCAGCAAGAATGCCAGCTGCTACTTTGACATCGAGTGGCGTGACCGGCGCATCACACTGAGGGCGTCCAATGGCAAGTTTGTGAC
CTCCAAGAAGAATGGGCAGCTGGCCGCCTCGGTGGAGACAGCAGGGGACTCAGAGCTCTTCCTCATGAAGCTCATCAACCGCCCCATCATCGTGTTC
CGCGGGGAGCATGGCTTCATCGGCTGCCGCAAGGTCACGGGCACCCTGGACGCCAACCGCTCCAGCTATGACGTCTTCCAGCTGGAGTTCAACGATG
GCGCCTACAACATCAAAGACTCCACAGGCAAATACTGGACGGTGGGCAGTGACTCCGCGGTCACCAGCAGCGGCGACACTCCTGTGGACTTCTTCTT
CGAGTTCTGCGACTATAACAAGGTGGCCATCAAGGTGGGCGGGCGCTACCTGAAGGGCGACCACGCAGGCGTCCTGAAGGCCTCGGCGGAAACCGTG
GACCCCGCCTCGCTCTGGGAGTACTAG

TABLE II

MOUSE NOMENCLATURE
ICSGNM Map3k6
Celera mCG20807
HUMAN NOMENCLATURE
HGNC MAP3KE
Celera hCG19300
MOUSE SEQUENUE - GENOMIC
GGGCTGGACCTTCACAGAGAGGCAGAGCTACACAAGGCAATACCAATTCTCTGGGGCCTGAGGGAGGAAATGCCCCAAACCCTGCTGGCTCCAGTTG
AGGTGGTGTGCCTCTGGGAGACAATGGGGAGAAGTCTTGCTTCAAGTTATTTCCCCCACAGCCTGATCCATCTATGCTGGCACCCCCCTCTAGTAGA
CCCTACAAGATGGGTCTACTATGAGCCCTACTGTTCTAGCTGCTCCTATTTAAAGGGCTGCCTGCCTGAGGGTACAGGGTCACCCCAGCTGCCCATG
TAGGGTCCTACAAATGCTCACCACATCAACCACTCATTCTCTCCTCATCTCAAGAAGCAGGGTGAGAGAGTTTGGTAATCTGGCCTTTTCATGTAAA
TTTATGTAAATAGGATATAAATATCACAACATATGTAAATAGGATATAAATACCACAGAACCTCAGCATCTCAGGAGGTCCTGATATCTAGAATCTA
GGAGGTAGAAGGAATGGCAGAAAAGTCTGGAGGAGATATGTTCGCCCTGTGCCCGTTGGCCCTGTGTTTCCATGTAGAACCCAGGGAGCTGGAGCTG
GAGCTGGAAGCCAGCAAAGTGGTCCCCCTGCCCAGTTATTCAGGTGTCCCAAGAAGTCCTAGAGAGAAGAGATCTGTAGGTTCTCAAGGTAAGGGAT
GTCAGGGACCTGGCAGAGAGGGCTCTCGAGGTAGCCTGTAGAGGCTGGATGTGGCTGCTTGGGTAAGAATGGCTCTCATAGGCTCATATATTTAAAT
GCTTAGTTATTAAGGAGTGGTACTATTTAAGAAGGATTAAGAGGTGTGGCCTTGTTAGAGGAGGTGTGTCACTGGGGGTGGGCTCTGAAGTTTCAAA
AACCCAACTCTGTCTTCCCTCTGCATGCCTTTGTGTGTATCTTTCTGTCTTAGGATCAGGATGTAGCCGGAAACTACTTCTCCAGCAGCATCCCTGC
CTCTGTGACCTCGTGCTCCCCACTATGGTGATTGTGGACTAAACCTCTGAAACTGTAAACAATCCCCCAGTTAAATGATTCTCTACAGGAGTTGCTG
CCTTCGTGCCCAGCACTCAGGAGGCAGAGGCAGGTGGATCTCTGTGGGGTCAAGGCCAGCCTGGTCTACAGAGTGAGTTCTGGGACAGCCAGGGATA
TACAGAAAAGCCTGTCTCAACAAACAAACAAACAAATAGGAAAGGTAGTCATGGTCATTGTGTCCCCTCCCAGCAATAGAACACTGACTAAGACACT
GGGGTGCAGGTGCTGAGTGTGGACAATGACGACGTGGGAACCACCTCTGCACCAGGCTCCAAGTGGGAGAGTGCCAGGATCCTGGGGTGTACCAGGT
AGATTGCATTGTTCATGTTTTACTGCAGAGGACTGTGAGGCTCAGAAAGGAACAATTGCTTGTCCCCAATACAGAGCCAGAAAAGGATGCTAATAGG
TGAGAACACCAAATTCTGTTTCCTCCTCTTCCTCTTCTTCCTTCTCCTCCTCCTCTTCCTCTTCTTCCTTCTTCTCCTCCTCTTCCTTTTGTCTTGA
GCACTGGGCAGGACACAGGACATCTGCCTCTGGAATTCCAGGGCTACCTTGGCTCCCTAGGCAAGATGGATCCCAAGGGAGAGTCTACCCTCTGACT
TTACTTTTTGCACAGACTAGGGCTGGGCCCTCTCAGCTACTTAGGGCTGTAAGTGCCCTCGGAGGAGCCAGCATTGGAAGGAGTAGTAAGAATTTGG
AGGAGCGGGGGCTGGAGAGGTGGCCCAGTGGTTAAGAGCACTGGCTGCTTTTCCAGAGGTCCTGAGTTCAATTCCCAGCAACCACATGGTGGCTCAC
AGCCATCTGTATTGAGATCTGACGCCCTCTTCTGGTGTGTCTGAAGACAGCTAGAGTGTGCTCATATACATAAAATAAATAAATCTTAAAAATAATA
ATTTGGAGAAGGGCTGGCTCTGCCCCATGCTGGCTCATGGGGCCTCCCTTAGGGTACTCAGACCTCAGAGTCAGTTGACACCTGGTCCTTTATCTCT
GGGCAGGACATCAGCCACCTGCTCTGCCACTAAGAGGCTAAGGAGGCGTCCACGGGAGTGAGGAACTTGGAATCAGGCCAGACGGGGGTGGTGGAAG
TTTCCAGGCATGTAAGCCTCTCCTCGTAGCTCTATAGTGCGCACCTCTTCCCCAGGTCCCAGGATCTGAGAGGACTGCTCGGTCCCTTACTTGTTCC
AGAATCTGGGTGGAGGCTTTTGGGAAACAACTTGGGGGCCTTTCTCAATCCACCACCCCCCCCCCTTTCCCAGGTCTCAAGAACTTTTCCAGTCTTT
CCCATTTTTTGGACACTCCAGGGGGTAAGAAAAAAAAGAGGCAACAGGCAAAGCCCGGTGGTGACTTCTCTCTAACTCCCTAGGGGGAGCCAGAGTC
CACTGAAGCCAGGCATACCTGACAGGACAGCTCCTACAGGGCCAGAAGTCTGAGAAGGCTTCATGGAGGAGGTGTTATTTGGAGGAAAGTAAAGGCT
CTGTGAACATTCTCTAGGTCTGTGAGTGCAGAGTACTTTGCGGGTAGAAATAAAGGCGAGCAGGAGGAGGTGGGAGAGAGGAAAGGCTAAGGAAAGT
AAGCTGGGGGTCGTGGGAGAGCCAAACATGAACCCAGAATCTTTGCAAAGGCCACTTCATAGAGGACATTGCATTACTGACATGGAGGGCAAAGGGG
AGCCATGGAAGAATTTTAGGCAGAAAAGGGTCTGGGTCAGACTTAGTTTTAGAAAGATAAGTCAAGAGGCTGGAGAGATGGCTCAGCGGTTAAGAGC
ACTGGCTACTCTTCCAGAGGTCCTGAGTTCAATTCCCAGCAACCCCACATGGGGCGACTCACAACCATCTGTAATGTGATCCAGTGCCCTCTTCTGG
TGTGTCTGAAGACAGCTACAGTGTACTCATATACATAAAAAAATAAGAGAAAGATAAGTGAGGCGTGGAAGTGCTTGCCTGTAACCCTAACACTTGG
GAGGCCGGGGGCAGAGGTATTGAAATTAGGGGCCTGGAGTGGGCCCAGGGAGGCCCTGTCTCAATAACAAGCGACATGGGGTTGGAGAGTGACTTTA
TGGTTAAAAGCAAGCATTGACTGCGGAGCGCCCCCACTGGATAGCTCATAACTGCCCCTACCTCCAGCTGCAGCAGATCTGACGTGCTTGTCTAGAG
TCCTCGAGCATCTGCGCACTGATGCACACACATTCTCTCTGTTAGACACATGACACCTATACTCACACGTGCATACACACACACACACACTCTACTC
TCTCCCTCTCTCACCTATTTTTAATCTAAAAACATAACAAAACAATAACAGGCTGCACCCTAGAAGAGACACTGGATGGGGCAAGGCAGAAGGTGGA
GACAGAGGTTAGGAATGGACAAGGCAGAAGACCAGAAGACCGTGGGAAGTGACATCCACAGTATTGGACATATGTCATCTGAAAGATACTGGAAGAG
CAGCAAGCACCACCCCCACCCCACCCCACCCCCCTAAGATTTCCCTGCATAGCGATGGAACTTGCTCTGTAGACCAGGCTGGCCTCCAACTCCCAGA
GATCCCATCTCCCGAGTGCTGGGATTGGGGAGCTTGCCACACCACTGGGCTGTCCTAAGAGCAGAGCTCCAGAGCTGGAGGACGACTTGGCTTGGGA
GAGGGAGAAAAGGCCACGGGAAAAGGCTAGGAATCCAGCTGAGGCCTCTGGGGGCAACATCTATGCTCTAAATATCTTGGTGGTTCTTCTTCTTCTT
CTTCTTCTTCTTCTTCTTCTTCTTCTTCTTCTTCTTCTTCTTCTTCTTCTTCTTCTTCTTCTTCTTCTTTTCCTTCTTTTCCTTCTTTTCCTTCTTT
TCCTTCTTTTCCTTCTTCTCCTTCTTTTCCTTCTTCTCCTTCTTCTCCTTCTTCTCCTTCTTCTCCTTCTCCTCCTTCTCCTCCTTCTCCTCCTTCT
CCTTCTTCTCCTTCTTCTTCTCCTTCTTCTCCTTCTTCTCCCTCTTCTCCTTCTTCTCCTTCTCCTTCTTCTCTCCTCCTCCTCCTCCTCCTTCTGT
AAGTATATATATATATATATATATATATATATACCATAGCTGTACAGATGTTTGTGAGCCTTCGTGTGGTTGATGGGAGTTGGACCTCTGCTTGCTC
CAGTCAACCCTACTCACTCAGGTCATCAGCTCATCTCTGCTCACCCAGTCCCTGCTTGCTCCAGTCCAAAGATTTATTTATTATAATACATAAGTAC
ACTGTAGCTGTCTTCAGACGCCAGAAGAGGGCATCAGATCTCACTACGGGTGGTTGTAAGCCACCATGTGGTTGCTGGGATTTGAACTTGAACTCAG
GACCTTCAGAAGAGCAGTCAGTGCTCTTACCCACTGAGCCATTTCGCCAGTCCCTAATACCTGGGTTCTTAACCTGCAAATTAAATGGGTAACTGGC
GACAGTATATATATATATATATTCATTCCAATCTTCCCTGGAGCTTACGAGATTTTTGTCATGAGTTGAGTGTCAACAAAAGTTAATGAAGAAACTG
GATTTATCATTCATTATTCATAGGCTAAACTCACTCAGAGGAGTCACATTTGAGCCGTAAGCCCCCTCCCCACAATGCAAAGAGAAGGGAAGGGTCC
AGCCAGCCCTCACACCCATCCAGCCCCTCGCTGCACTTCCCTGGCTTCCCCTGCCCCAGGCCAGTTTGGAT1GGCAGCAGGTGCAGGGGTGGATTTC
TTCCACTCCTGACTGCTACAGAGCAGTGGGGAGACAGGAGTCTGCGTTCTGGCATGGGAATGAAAATTTACAGGAGCTTCCTCGGTAGGGTGAATTC
CAAGTGAGAGGGCTGGGCCAGCCCAAACCCCTACCATCTCAGTCTCCTCTTCCTCCCAGGTATCTGGGAAGGTCCTGTAGAGTCTGAAGACTTCAGC
AGTAGCTGCATCTTTGTTTATTATGCTACCTTCTGCCTCCTCAGGGAGGAGGAGCGCTGGAAGAGTACATGCTGGGTAAATTCTCAGAGGGCGCTGC
AGGTGAGTGGCCTACAGGGGGTCCTGGTGTAACTTTAATTTTTTATTTTTTTAAATCTTATTTTTAATGATTTCTTTTAATGTTTTAACCCGTTTTT
TTGTAACCCACCAGAGGTAGTGGAAAAGAAAGAAAACGGGAGAAATGGACCTATTTAGAAAGGTTTTTTGGAACAACAGATGTCTGTGTTATCTGGA
AATCAGCAGTTCAGTTCACAGGTTAGCAGGCATCGATGGCTTGATCCACTAGCAAACACCTCACAGACACACCAGCAGGCCAGTTCAGTACAGTCGG
GTTAGCAACGGTGGTGACACGACCTAGTAGAGACAGCCAGGCCTCAGCCTCAGCTTCAGTCAGCAAGAACGACACTGGGAGAAGTTCTCAACTGTGC
CTCTCTCAAGGAAAGCCACGATCAGCAAAGACTCAAGACCCACAAGCGGTGTGTAGCTAGCTCTATACGCAAATCTAGCTCTGTCTCCATCACTCCG
TGGCGTCCTATTTATACCCTCCAAACATCACGTGTCCTCCATATGCCTTGTCTTGCCTCAGCACAGGCATCCAATCAGCCCCAGTCCTTAGAAGCAA
CAAACAAACTGCAGCACACCACAAGAGTTTTGGTGTGTTTCTCTTTATGGAGTTCCGACAAAGGCAGTTCAACTACACAGTATAAGGCAGACCAATA
CATGCACGTTGTTAGCAAAAAAATCCTTCATCACGTGACTTTTCACATTCTTGCTTTAGCAGAACATCCTCTCTCCTGTGTCTGCTTCAGCGAAACG
TTCCTTCCTGAGTCTGCCTTAGTTAGCCTTTCACACCTGTGTCCACTTTAACAAACTGTTCCTTCACGTGTTTGCCGAGCAAAACACCATCCGACCG
ACCTTTCAAAGAACCCTTAAGTTTCCACTTCACCCGGGGATTGGGGGGGGGGCAGTGGCAGAGGAACAGGAAGGCTGGCTATTCTCTATATTGGCCC
AACATTCATGAATGAGCTCAGCTAACTTCTTCTCTTCTGTGGGCCTCAGTTTCCTCATCTGAGAAATGGCAGTCTTGAGACAGGGTGGGGCCCGAGG
AAAAGGACTCGGTTCTGGGGAGGAACAGCTTCAGAGTGCAGAGTTACTGTGTATTTACTGTGGGTCTCTGGGCACAGCATCAGTTTTGCCTTACACA
AAATTGATGGGGGTGGTGCACGCAGTGTAGGAGTGGGGGGCACACAGTGCAGGGGCTGGGGGCACACAGTGCAGGGGTGGGGGAATGCAGTGCAGGG
GTGGGGGAATGCAGTGCAGTGCTGGGGGGCATGCAGTGCAGTGCTGGGGGGCATGCAGTGCAGGGGNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNGGCTGGGGGGCACGCAGTGCAGGGGTGGGGGGAATGCAGT
GCTGGGGGCATGCAGTGCAGGGGTGGGGCTGTTATCTGCTGCTTTGGTGTGTGTGCACCAGTGTGGTCTGCTCCAGGAGAGGAACCGGCAGACTTCT
TTCAGAGAAGCAGTGTTAGGAGATAACTGAAAGGGGAAGTGGTTATGGCGGGGTGCCTCTAAGGAGCCCCCTTTCCTGCCCAGGCCCAAGAGGTTCC
TCTCAGCTCTCCCTGGCAATGTTGCTCAGGCTGCTTGCTGGGACTGAGAGTAGACAAAGGGACTGGAGAGAAGCTGGGAACTCTGGAGGGGATAAGA
CTTGGGTCAAGGGGGGAGGGTATAGGGGTCTTTCGGGATAGCATTTGAAATGTAAATGAAGAAAATATCTAATAAAAAAAAAAGACTTGCTGGGCGT
GGTGGCGCACACCTTTAATCCCAGCACTCGGGAGGCAGAGGCAGGCGGATTTCTGAGTTCGAGGCCAGCCTGGTCTTCAAAGTGAGTTCCAGGACAG
CCAGGGATATACAGAGAAACCCCTGTCTTGAAAAAACAAAACAAAACAAAACAAAACAAAAGACTTGGGTCAAACTGATGGCTTGGAACAAAAAAGT
CCCACTTCCACTGTGACTAGCTGAATGACCATAGACAAACAAACCAACAAAAATTGGCTCCTTGGTGCTGTTGGCTCTCATGGGGCCTGATAACATT
CACCTCATTACTGATTGGAGGCTGCCTTGGGGTAATATCTGTAAGGGCTTCAGGCATCCTGTAGGGTGCCAAGGTGTCTTCCACTCCTTGCTGCAGG
GGACTCTCATCTTCCACAGTGGGAAGACTTTTTTTTTTAAGATTAATTTATTATTACATGTAACTACACTGTAGCTGTCTTCAGACACATCAGAAGA
GGGTGTCAGATCTCATTATGGGTGGTTGTGAGTCACCATGTGGTTGCTGGGATTTGAACTCAGGACCTTCAGGAGAGCAGTCAGTGCCCTTAACTGC
TGATCCACCTCTCCAGCCCAGTGGAAAGACTTCTACCACTGATGGTGCAGAGCCTTATTCAAGATGGGACAACTGTGCAGTAACTGTCCCTGGTGCT
GGGTTGTACAGATCCTGCAACTGAACCAATCTCAAGGGGCATTATGCTGCATCTGAGGCCACTGCCCACATGTATGGCATTGACTGGACCTCAGAAA
AAGGCGTGGGTCACCCCTACTGTGGGATTACACATAATGCTTTGCTAGAGCACTCAATTGTCAGTATCGTTGTCACTTCTTACAGCTTCCATCTCTT
GGCCACCTCACTGCCAACTGCCTCTGCTTCCAGGGCCACTTTGTAACACTTTACAAATCTTTTCTTACTCAGGAGAAAACCTATAGAAAACGGGGCA
GGGACACAAGCTTCAGTTCCATTCAAGGAAGAACTTCTAATCAGGAAGATGAAGGGTTGAACTTCTCAGGGCTTCAGACCCTAGAAGGATACAAGAA
ATGCTAGGGGGCAGTGAGGTGAGAGATTCAACCACCCACTCGCAATCCTCCTGGGCCCCCGAGCCCAAAGTGGATGCATCTGAGGACAGCTTTCTCT
CACCCTTTGTTTGCTGTTTGGTTGGTTGGTTGATTTTGTGAGACAGGGTTTCACTCAGCAGGCAGCCCTGGCTGTCCTAGAAACTCTGTAGACCAGG
TTGGCCTTGAACTCAGAGATTCACATGCCTCTGCCTCCTGAGTGCTGGGCTTAAAGACATGTGCCATCACCACTTGGTTCCCATTTGTTTCTTGTAA
AGTGTGTTTTCTTTTTTTCTTTTTTTTTCATTTTTTATTAGGTATTTAGCTCATTTACATTTCCAATGCTATACCAAAAGTCCCTGATAGTGATGAT
CAGAACATTGTCTCCCTGATTGAAGAGGACACTGGGATTGGGCTCTGCTATAAACCAGACCCCCCCATCCCGAGACCCCCAAATCTCCTGATATTAC
ATCCTGAGCCTCTATGAGTGTAAAGGAGCTCGTGTCTGATCTTCCACAGACGTGACTTTTTCGCCTTTAAGGGACCTCTTTAATAGGTCCCCCACCC
TCTGCCAAAGTGTGCTCTCTCCCACCATTANNNNNNNNNNNNNNNNNNNNTCCCCCTTTTTGGCCCTGGCGTTCCCCTGTACTGGGGCATATAAGGT
TTGCGTGTCCAATGGGCCTCTCTTTCCAGTGATGGCCGACTAGGCCATCTTTTGATACATATGCAGCTAGAGTCAAGAGCTCCGGGGTACTGGTTAG
TTCATAAGGTTGTTCCACCTATAGGGTTGCAGATCCCTTTAGCTCCTTGGATTCTTTCTCTACCTCCTCCATTGGGAGCTAAAGTGTGTTTTCTACC
CAAACCTCTGTAATGGGCCACATTCTCCCTTTCTGTTGCTCGTCTATCCCATTGGTTCAAACCTAGGAAGATATATTTTTTTCCTTCCATGAGACAA
GAAGGCTGTGAAACAGAGGCTTCCTACCGTTTGTTGAAAGGTCAATTTGTCCAAAGCCCCAACATATTATTTTATAAAGTTTTAGTTTTATTTCATG
TGCATCTTTTGCCTGTAATTATATATGTGCACCAAGTATGTGCTGGGCGCCTATGCAGGTCAGGAGAGTGCCAGGAATCCCCTGGGCCTACAGTTAC
AGATGATGGTGAGCCACCATGTGGGTGCTGGGAACCAAACCAGGGCCCTCTGAAAGAGCAGCAAGTGGTCTTTACAGCTGTGCTGTCTCTCATGCCC
AAGCCCTACCATAATAAACAATTATGAAACATGGCCCTAACCTCAGAGTCTCAAAGTCTCATTTCTAAGTTCAGGGCCCTCTAAGGTTTATAGCAAC
TTAGATTCATACTAGGATGTTAAAGCTTCTTGGTGAATTTGAAGCCGGGATAACCTTGTAACCCAAAAAGCGGAGACTTCTGGAGTTCCTTCAAAGG
GTAGATAGGAAGTTTACCGTAGAAGTGCTTTTCAGCTCCATGGTCAAGTTTCGGGTGGACCAATAAGGTCCACAAGGTTTCACTACAAGAGAGGCTC
TGAGGAAAGCGCTTGTCAACATTCTGACCCCTACCAGCAGGAACATTACATGTGCCGCCTACACAATGTTTTGCTCCCCCGTCCTTTCCCCGCCCAG
TGTCCATTCATTTACCAGGTCCCAGCAGGGCGGGGCTGTCCTGTTTTCTATCCCTGGGCTGGGCACACGTCTTACGAGCGCCTGCAGTAAGCAGTCA
ACAGTGTTTGGCCCGAATAGGGGCACAGGCTGTTCCGGAAGTGGACAGTGTCAGAGGTTCTAGCCGGAGAGGAGGGAGGGAGAGCCGCTGGGGAGCC
TCATTGCCCCAGGGCCGAGGGTGGAGCCTTTCCCAGTCGGGAAAGGGCTGGGCCCAGCCTACATGTGAGCGCAGGACAGAGTGACTGCCAGCGCTCC
CTTGAGAAGTCTGGGTGTAGCACGCTGGCGGTTGACTTCCCAGGACTCTTCTGTGACCTGTGAGCCGAGGATTACTGGGTACCCCAGATTCCCAGAA
CCCAGTCCTGGATTGCAGATCTGTAAGGACCGCGAAAGATTGAGCAAAGAAGTCTTTCTGGACTTAAGAGTTCCGGAACAGGACGTCCAGCCCATCT
CCGGATCCCGACTGGATCCACACCTTCAGCCTCGTGCCCTGTCCCGGGTTCCCCGCGCCCGGGGCCCCGCGCTTGAGCGCCCCCGCATGGCGGGGCC
ATGTCCTAGGGCCGGGGTTCTAGAACGGGCGGGCAGCTGCTGGCAGGACCCGCTGGCGGAGGCGCTGAGCCGGGGCCGCTCGTCTCCTGCGGTCACG
GGTCGGGGCTGCGCGCGGAGCCGGCCTCTCAGTGTGGTCTACGTGCTGACCCGGGAGCCGGGGCCCGGGGTGGAGCCCGGGTCGGGAACCGAGGCAG
AGCCGCTGCCGTTGCGATGCCTGCGCGAAGCCTGCGCTCAGCTCCAGGGCACACGGCCACCCCCGCAGCTGCGCAGCCTACCCTTTGCGACGCTGGC
GCTAGGCGACACCGCTGCGCTGGACTCCTTCTACAACGCGGCTGAGCGACTTGGGGCGGGGCTTGAGTCTAGGCGGAGCAAGGGGTGGAGCTTTAGA
ACCCTGACGGGGCGGGTGTGGGTACCAGGTCTAGGGAATTTACGGTAAACTAAAGCCGAACTTGAGACAAGGCAGTAACGACAGGGCATTTAGTGGA
TTATGGATGTGATGGGCTTGAGGCTGGATCTGGGGATTCAGGCCTGGCTCCGGAGTTTAAGTATCTTTCGGTGAGCGTGGTGCCAAGCAGGTGGGGC
GGAGCAGGCGAACTCTAAGGGCGGGGCTGGGGAGGGACGGGGCTGAGGGAGGGGCAGAGCTGCGCTTCCCTGCGTAAGCGGGAGAACTCTTTTTCAG
GATGATCTCTCGTTGGACTTTTGAGGGTGAATCAGGCCTGAGACGGAAGTTCGGTCTATGGCAAGGGGCCATCTCTGCAGGATTGGGGTTGATAAGT
CCTAGCATACTACATCCCACATGGGACTAAAGGGGCGGGAAAGGAAGTACTGAGGCAACTCTTCGGGACTGGATTATAGGAGAAAGGGATTGTATTT
GGAGACTAGGAGTCGAAAGAAAATGGGTGGGGCAGGGGTGGGTCTAGAGGAGACACAGGGAGTCCAAGGGTGAGGTTGGAAGATAGCTATTCCTCTC
TTTCCCACCTACCGGATGCTCCACCTCATCCCTATTTGTTTATCTCCGAGTCAGAGAGCCTTGTATCCTGGACATCTGCTAAATTGTAGCTTAAAAA
TGAAGGCTGACCCGGTCAGACCACAGATAAAGTGGAATCATCTGCGTTGACTCATACAGTCCCTCCCTTGCGGACATTGTCACTTAAAGATGGCTGG
ATACAGTAAGCTACACAAACACACATAAAAGCACGACGTTGTCTTCACACACGATGATGCAGATCACCCTCAGGACCCTGGCTTCAAGATTGGAGAG
ATGGTTCTGAGGTTAAGAGCACTTGCTGCTCGCCCAGAGGACCCGAGGTTCCCAGCATGCACACGGGGTGTGCATGCTCACAACCACCTGTAACTTT
ATCTCCAGAGGATCCTGATGCCGCTTTCTATCATACACTCACACCAACACACACATACATATAAATAAACAGAGAGAGAGGAGAGAGAGAGAGACAG
AGAAAGAGACAGAGAATGGGACTTCTTTTTGCAAAGTTCCTGTCTCCTACATAACCTGGAAGTGCTCAGTGGGATGAAGGTCCCTGGGCATCTCCTG
TAACACCACCAATAAGAGTACTGTGAAAACCCCGGGAAGCTCTCGAGAGCAGCACCGACTTTGGCATGAGGTGCAGGCAAGGCACTTGACGTGACAT
GCATCTCCAGGCTCCTTCGCCACTGAGCTGGGCTTCCACACTCAGGCCTACACTTCTGTGTCCACAGATGTAGTGGTGCTGGAGGTGAGCAGCTCCC
TGGCACAGCCTTCCTTGTTCTACCACCTCGGTGTGCGTGAGAGCTTCAGCATGACCAACAACGTGCTCCTCTGTTCCCAGGCAGAGCTCCCTGATCT
GCAGGCCCTTCGTGTAAGTGGCCAGGGGGCTGGGGGCTGGGAAGACAGGTGCAGGGTTGGGGTGAGGCCACCCCAGAATGCCAGTATCTGATTGTCT
GCCTCCTGCAGGAGGATGTTTTCCAGAAGAACTCGGTGAGCAGAATCTACCCCTCATGCCAACGTTATAACCTCTGACCCCTACTAACCTTCCTAGC
TTCCTCCTTTGTCTCTGACATCAGTGTGACCCTCAGCACCATGTAGCATCCTTTTGTGACGTGCGGCTCTTTTCTTCTATTCCTATCTTCACCAGTA
CTAACCGCCAGTCCGTCTCTAACCCTGTGCTGACCTATGTGTGACCTCCACTAATTGCTGCCTTCTTCCCACCAGGATTGTGTTGGCAGCTACACAC
TGATTCCTTATGTGGTGACAGCCACTGGCCGGGTCTTATGTGGCGATGCAGGCCTCCTGAGGGGCATAGCCGATGGGCTAGTACAGGCTGGGGCGGG
CACTGAAGCCCTCCTCACTCCCCTGGTGGGCCGGCTTGTCCGTCTGTTGGAGGCTACACCCACAGACTCTTGGTAATGGGGCTCAGGGGGAGGCGGT
ACTAGGTAGAGACTGATGAAGGTAAAGTGTGCTGGGGCTGAGGGGCAGGCTCATCTCTCTCTCTCTCTCTCTCTCTCTCTCTCTCTCTCTCTCTCTC
TCTCTCTCTCTCTGGTCTCTGGGCATCAGCGGCTATTTCCGGGAGACCATTCGTCAGGATATCCGGCAGGCTCGGGAGCGATTCAGTGGGCAGCAGC
TGAGGCAGGAGCTGGCTCGCCTGCAGCGGAGGCTGGACAGCGTGGAGCTGCTGAGCCCTGACATTGTCATGAATCTGCTGCTGTCGTACCGCGATGT
CCAGGTGTGTGGCGTTCGGAGAGGCCATGACATAGGATATAACTCCAGCACTCAAGTGCTTGTGTCAGGAGGGGCAGGAGTCTGAGGTCATCCTGTT
TACAAGCAAGTCTAAGGCCAACCTGGGCTTCAAGGAATCTTGTGTGTAAACCTAAGAAAGAGACCATTGTATGACAGACAGATACAAGTGCAGCTCC
TGGCTGAGCCTCACTAGTGGCTTTAAGTAAATTGTCTGAGCCTCAATTTTCTTATTTGTTTCATCATCATTATTATTTTTTAAACATGGTCTCACTG
TGCATTCCTGGCTGGCCTGGAACTCAATATGTAGACCAGGCTGGCCTACATATCCACCTGTTTCTGCCTTCTAAGCCTTGTGCCACCACACCTGGCT
TTATTGTTTTTTTTTTTTAAAGATGCATTTTATTTTTTTAATTGTGTGTATATGTGTGTGTCTGAGTTAGGGTTTGTACCTTGTGTGTGCAGTTACC
TGTGGAAGACAGGAGAGAGTCTTGGATCCTTTGGGACTGGAGTTAAAAGAGGTTGTGAACTTCCTGTTGTGAGTTCTGGACCCCAAACTGCAGTCTT
CTGGAAGAGCAGCCTGCACTCTTAACTGCTGCTAGTTATCTCTTCAGCCCATATTTCCTTCTTTTTTGAGGGATGATGTGGTCATTGCCTCACAGGG
CATTGGTGAGGTTAGAAACACCCAATGTGGGGTGCATAGTGGGTTCTCCAGGAAGGTGTGGTCTGTAAATCTGCTTGGCCCGAGGGAGGTGTGCAAA
GTCTTCAGGTTGTGGGTAGTAGAAATTTCCTTCAAACTGACAGTAGTTACCCCTGTCTTAAATCCCAGCACTCAGTAAGCAGAGGCAGGCAGATATC
TGAGAGTTCAAGGGTAGCCTGGTCTACAGGGTGAATTCCAGGACACCCAGGGCTATGTAGAAAAACCTCATCTCAAAAAAACAAGCAGAAAAAATGA
TTTCTGCTGGCAGTGATGGTGCGGGTCTTTAGTCCCAGCACTCGGGAGGCAGTAGCAGGTGGATCTGTGAGCTTGAGGCCAGCCTGGTCTACAGAGA
GAGTTCCAGGGCAGCCAGGGCTACCGATCTTTCCTCGCTGTCAAGGGCTCATCGAGATGCTGCACGCTGTCTCCCACAGGACTACTCGGCCATCATT
GAGCTGGTGGAGACGCTGCAGGCTTTGCCCACCTGTGATGTGGCTGAACAGCACAATGTCTGCTTTCACTACACATTTGCCCTCAACAGGTGGGTAA
TGGGGCACGGCTTGAGTCAGAGTCTTGGAAGTCTCTGTGACCTGCCAAGCAAGAGCACTGCAATCTGGAACCACTGCTCCTCGGGTATCTTGCTAAC
CTGGGTCCTAATGTCACAAGGTTCCTGTTGGTCTGGGCTAAGGATGCATGGTGGGGACAGAGGACGTGGGATCCCCTTGATAGGGTCATAGCTGTCC
AAGGTCGCTACTCCTTAGAGTTGTTTGCAACTAGAAATCCCTTCCATCTGGAGTCACTAACTCTAGAGATCAATGCTTCCCGGGGTTAGTGTCATTT
GAGGCTCATTAATAGTAAGCCCCCAGAAGGGTCCCTTCCGTTTATGGTCTATTGGGTCCCAGGACTGGGTGGGTCAGAGCCATCAGACAGGTCTTCA
GTGTATGTTCCTCCGCCTTAGGAGGAACAGGCCTGGGGACCGGGAGAAAGCCCTGGCTGTCCTGCTGCCATTGGTGAAGTATGAAGGTCCCGTGGCT
CCGGATCTGTACTGCATGTGTGGTCGTATCTACAAGGACATGTTCTTCACTTCTGGCTTCCAAAATGCCGGGCACCTGGAGCAGGCCTATCACTGGT
AATTCACATAAGAGTAAAGAGAGGGCTTAGCGATGGGACTGAAGGGTGGGGTTGCGGGCATCCATTCAGCCTGGCATCCCCTGCCCGACCCGACTCC
AGGTACCGCAAGGCGTTTGATGTGGAGCCCAGCCTGCACTCAGGCATCAATGCAGCTGTTCTCCTCATTGCAGCCGGGCAGCATTTCGAAGACTCTG
AGGAGCTCCGGCTGATTGGTGAGGCCTGCTGTTAGCCCACAGCACCCTGCACAGGCTCCAAAGGGCAAGGAAAGGGCCCCAGGCGGGAAAAAGCGCT
GACTCAAAGTGGGCTTCGAGACTGATCGGTGCCTGAGGTCTGAGGTCTCAGGAGCCTTTGAGTGCCTCTCTTCCCAAGCCCACACTTCCGGTTGTCA
CACTTTATCCCACATCTATTTGTGCCCTAGCTCTCCCCTGTGCTAGGCTCTTAGCGCTGAGATCTTGCCACACCTACCAGATCTACATTCATATCCA
CACTCCTGTGCGTGAGGGCCACAGTGGGACCCACACTTGAGCCTTACTCACTCCGGGACCTACACTGGATTGAGCCCACGCCACCCTCCCCCAGGCA
TGAAGCTGGCCTGCCTGCTAGCCCGCAAAGGCTGTGTGGAGAAGATGCAGTACTACTGGGATGTGGGATTCTACCTGGGAGCCCAGATTCTTGCCAA
CGACCCCATCCAGGTGGTGCTGGCTGCAGAGCAACTGTACAAGCTTAATGCCCCCATTTGGTAGGTGGCCCCTTCTTCAGCCCTGCACCCATGCCCG
TTGGCACCCCTCCCCAAGCCATGACCCCAATACTTTGATGTCACTACGATAGATAGACAGTGCCCGTGTTCCCCTCTGTTCGTGGTATGTGATGCCC
GTATCCTGACTACATGCAGCTGAGTGGCACCAGCCGGCTCCTAGGCTGAGCTGAGGCCAAGGCTGTCTGTGCATTTAGGTACTTGGTCTCCGTGATG
GAGACCTTCCTACTGTACCAGCATTTCAGACCCACACCAGAGCCCTCAGGAGGACCCCCGCTTCGAGCTCACTTCTGGCTCCACTTTCTGCTACAAT
CCTGCCAGCCTTTCAAGATGGCTCCTCCTCAGGAGGACCAGTGCCTGGTAAGGGCAGTGGGGCCTAGGGACCCAGGGGCCCAGGGACAAGCACATTA
TGACCGATCCCTTTCCCCGCTACTGCTTACAGGTACTGGTGCTGGAGATAAACAAGGTCCTGCTGCCTGCCAGGCTTGAGATTCAGGGAACAGACCC
CATGAGCGCAGTGACCCTAAGCTTGCTGGAGCCAGAGACTCAGGTGAGCTCTGAGACCCATTACTCACCGATCCACCTTTCTTTCTCCTATCCTCTC
TAGTCCCAGCGCCTCCACAACCTCCACCCCTTTCTTCTCACAGGAGGATCCTTCCAGCTGGACCTTCCCAGTCACCTCCATCTGTGGGATCAGGTGA
GAGCTGAACCTTCCGGCAAGCTCCTGTGCCACCAAGCCTGGCATTCAGTAGCCTCTCTTCTTCCCTAGCACCTCCAAGCTGGACCAGCGCTGCTGCT
TCCCTGTACGTACTTCCTCCGGCCCAGGACGTCCAGCTGTGCTTTCCCAGTGTGGAGCGCTGCCAGCGGTGCGCTCAACCTTTCTGAGTTGGGGATG
GGACGCCCAGGGGACGGAACGGACCAGGTGCTCCTGGGGGATGGTTCCTGGCTGTTCCCCTTCCAGCTGTTTCCCCCTCTTCAGGTTCTGTGGCCTA
ATTCACGTCTTGGTGATGAATCCGGATTCCTCCGCGCCCACTGAGGAGGCAGAAGGCGCAAGGGAAGTGCTGGAGGTGAGGCTGCAGAAATGGGGGG
GTTGGAGGAGAGCGAGGGGTGAAGGGTCCCTGCTAAGCGGGTGCTCTGGGTTCCACAGTTTGATTATGAATACTCGGAGACCGGTGAGCGGCTGGTG
CTGGGCAGAGGCACATATGGGGTGGTGTATGCTGGCCGAGACAGGCACACGAGGGTACGAATCGCCATCAAGGAGATCCCAGAGAGAGATAGCAGGT
GGGGTGCATTGGTCGATGAAGGTCTGGGGTCTCGGGAGGGTATATGCAAACAGGGTGTGTGTGTGTGTGTGTGTGTGTGTCTGTCTGTCTGTTGAGG
AAACGTGAGAACTGACTGATTGTGGGCATGGAAAGAGCTCTTTAGTGGCTGGACTAGGGGTGGTGGCCCTTACAGTAGATAAGAGAGGTCTGTGGGC
GGGGTGTTGGATCTGGGGGCATGGTTTCATGGTACAGGACCATGGGAGGTTATCTCTGCTCACATCTGCCCATTGCTCTCTCCTCGACACTCCCAGA
TTCTCTCAGCCTCTGCATGAAGAGATCGCTCTTCACAAACGACTTCGTCATAAGAATATTGTGCGTTATCTGGGCTCAGCCAGCCAGGGCGGCTACC
TCAAGATCTTCATGGAGGAAGTGCCTGGAGGTACCAGCCCTGCGTGGGAGGGGAGTGAAACCTGAGGTGTGGGGCGTGGGAATGGAGCCAGACCAGG
GTGGCAACCCCTGAGGGGAAATGAACTCTAGCGTGCGTGGGTATCACCTAGAATTAGCTGACAGGGCTCACTGGGTGATGGAAGTCTGGGTCATGGG
AATGGGTGTGTTTGGGCTTTGATCAGAGGGATGTAGGTAGAGTAAGCGGACATCAATTATAAACTTGGTTTGTCTCTGCCCAGGCAGCCTGTCCTCC
TTGCTTAGGTCAGTGTGGGGACCCCTAAAGGACAATGAGAGCACTATTAGCTTCTATACACGTCAGATCCTACAAGGACTCAGCTACCTCCATGAGA
ACCGCATCGTTCACAGAGACATCAAGGTCAGCCCTGTGGCTGCTCCGGGTGTGTGGAGACACAGTACCAAACTGAAGCTGACAGTAATGGTGGGAGA
GAGCACACTTTGGCAGAGGGTGGGGGACCTATTAAAGAGGTCCCTTAAAGGCGAAAAAGTCACGTCTGTGGAAGATCAGACACGAGCTCCTTTACAC
TCATAGAGGCTCAGGATGTAATATCAGGAGATTTGGGGGTCTCGGGATGGGGGGGTCTGGTTTATAGCAGAGCCCAATCCCAGTGTCCTCTTCAATC
AGGGAGACAATGTTTTGATCAACACCTTCAGTGGGCTGCTCAAGATTTCTGACTTTGGCACCTCTAAGCGGCTGGCAGGCATCACACCGTGCACGGA
GACTTTCACAGGTAATGGGGGGTGGGGGTGGGGTGAGGGTGTCAAGCTCTTGGAGACTTCTGGGGAATGGAGTTTTTTTTCTTGGCACAGAGTTACA
ATTTTAAAGATTTATTTATTTATTTTATGTATATGAGTACACTGTAGCTGTATAGATGGTTGTGAGCCTTCATGTGGTTGTTGGGAATTGAATTTAG
GACCTCTGCTCTCTCTAGTCGGCCCAGCTTGCTCAGTCCCTGACTGCTCTGGCCCAAAGATTTATTTATTATTATAAATAAGTACATTGTAGCTGTC
TTCAGACACACCAGAAGAGGGTGTCAGATCTCATTAAGGGTGGTTGTGAGCCACCATGTGGTTGCTGGGAATTGGACTCGGGACCTTCGGAAGAGCA
GTCAGTGCTCTTACCCGATGAGCCATCTCACCAGCCCTTGAATTACAGTTTTAATGGTGAATGGAAGGAATCTGGCTGGAGACAGGTGACTGGATCC
AGGAACCGCTGGTAAAATCTATACTCCAAGGTTGGGTAACTAATGAGAAAGACCTCAGCATCGAAAACGGATGCAAGGCCGCGTGTGTTGGGGGTGT
GAGACGGGTTGGTAGAATTTGAAGGGAGGACGATGTAGCGTCACGTACAAGCCAGAACCCTCCTGATGGTTGAACGCTATGCAGCCTAGAATGAAGG
ACGGAAGCTTGGCAGGTGACGGGGCCTAGGTGTGCACATGGCCAGTGGATGATGGGGACTTGATGCCTACCTGGCATTGTTGTTCCTATTTGTGGTC
TAGGGACTCTGCAGTATATGGCCCCGGAAATCATCGACCAGGGCCCGCGAGGATATGGGAAGGCAGCTGACATCTGGTCTCTGGGCTGCACTGTAAT
CGAGATGGCAACAGGTCGACCGCCCTTCCATGAACTAGGGAGCCCTCAGGCTGCGATGTTTCAGGCGAGTCTCTCTGGGGTGTGGCCTACGGAGGGT
GGGCGGAGCCAGATGGGGCCAGTGGGGTTATCTATCCCATCTGCCCCACCTCCGCAGGTGGGCATGTACAAGGTACATCCACCCGTGCCCGGTTCCC
TGTCAGCTGAGGCCCAAGCCTTCCTCCTCCGAACTTTTGAGCCAGATCCCCGCCTCCGAGCCAGTGCCCAAGAGCTGCTGGGAGACCCCTTCCTTCA
GCCAGGGAAGAGGAGCCGCAGCCCGGGCTCTCCTCGGCATACTCCCCGGCCCTCAGGTGCTATGGGGGTGGAGGGGGACAACCTAGTGAAGGGGAGA
GGGGCTGATGAGGCGAAAAGACTAACAGCTTGTTCCTCCCTCAGGCGCCCCTTCCGGTCCTTCCAGTCCTTCAGCTGACTCAGCCACACAGTCTCAG
ACATTCCCAAGGCCCCAGGCACCCTCTCAGCACCCACCCAGCCCCCCAAAGCGCTGCCTTAGTTATGGGGACACCAGTCAGCTCCGGTGAGCGCCTA
GGGTCCTGGAGTGGGTTCTCTTCATCTTTTTTTGCCGTTGTATTAATTTCCTCCAGAAAAAAAAAGCCTCCCTGGGTCGCAAACACCCTGACCACTT
CCAAGGACCGTATCTACTACCTCCTGCCCTCTCCCAGATGCCGGTGGCATTCACTCTGCCAACCCCCAGATGGGCTTAATTTCTATTATCCCCGTGA
AGTAGGCATAAAAGAGCCAAGATTCAAACCCTGCAACATAGGTGGTTGGTTGCATGGTGTTGCCCACCCTCCATCTAATGCCCCTCATTGTGCCCAG
TGTGCCCGAGGAGCCCGCTGCCGAGGAACCCGCGTCCCCAGAAGAGAGTTCAGGCCTGAGTCTGCTGCACCAGGAGAGCAAGCGCCGGGCCATGCTG
GCTGCGGTGCTGGAACAGGAGGTGCCCACACTAGCAGAGAATCTCCTGGAACAGGAACAGGTGGGCGGCCCGCCCCAGCGCCCCCTGGTGGTCGAGC
TGGGATAAGAATTTATAGTGCACCGGCGAGCATCTCTGTTGACTGCGGATGCGAACATCCACGGACGTCCCCTCTTTTTAGGACTCTCGACTCAGCA
AGATTCATGTGGAACTTCTGCTTCGGTGCCTTGGGGCACAAATCCACACTCCTAACCGCCGGCAGCTGGCCCAGGAGCTGCGGACCCTGCAAGCTCA
GCTGCGGGCCCAGAGCCTGGGCCCTGCGCTTTTGAAGGGGCCGCTCTTCGCCTTTCCAGACGCGGTGAGGAAGCTATCTCGAAGTTAGGGCTATGCA
ATAACAGGTCGCGAGCAAGATCTTGATGTTCCAGGATTCTATGATTTCCAGGAAGACGCATGTCCAGTCCTGGCTTCCCTGTCCCACCCTTGACCCC
AAGTCCGCCCACCTCTTTTAGACTGCAGCCGACTTTCTAGGGCTTTGCAACTCAAGCCCCACCTCTGTTTGAGATTCAACTGTCTCAACCGCGCCTT
CCTTCCCTCCCCAGGTGAAGCAGATCCTCCGCAGACGCCAGATCCGCCCACACTGGATGTTCGTGTTGGACTCGCTGCTCAGCCGTGCAGTCCGGGC
GGCGCTGGCGGTGCTGGACGCGGGTAGGTGGGGTTGGATCCCGCCGGCCGGCCGGGGCGGGGTGCGTGGCCCTGGGGGCGTGGCGAACATAAAATTG
GTACTTCGCGGCCCTCCCTTCTCCCTGACCGTCTGGGGTCCTAAACGGGCGCGGCGCCCACCTGGAGCTCAGTCTACTCAAATTGGTAGAGTCGGAG
AAGAAAGCGGTCTTACCGAGGTCAGAAGAGTCGAGTAAAGAGGAGTCCCAGCAGAAACCTCAGGAGAGCCAGGCGCTGCAGAGCCAGCTCCCACCAG
AGCAGGGACCCCCATCGTTGATGGTGGAGTTGGGCCTTTTGCGAGCCGAGACTGACAGGTGAAGCCTTTTGGAATCACTCGGATAACCTGCAGAGGC
TTCAATCTGCAGGCCCTTCCATTCTACCACCCCTACCTTCCCACAGGCTTCGGGACCTTCTGGCTGAGAAGGAACGTGAGTGCCAGGCGCTGGTGCA
ACAGGCCCTGCATCGGGTGCATGCAGAGACCAGGAAGTATGCCCCAGCTTCCGAGACCCCAGGTGAGCCGACTTGACTGGGAAATGGTGAAGGTAAG
GCCACAGAGGACACCTTTGCCCTCCCTCATCACCTGCCTTTTTGACAGCCACTCTCCCAAAGGACCAGAACCTGGTGCGGTGGCTACAGGAACTGAG
TGTAGATCCAGCCACTATCCAAACGGTGAGTAAGTGTGAGCTGGCTCCCACCACCCACCCACCAAAGGCATCTCCTCTGTCTTCCAGCTATTCAAAC
TTTGTCATTGCTGCAAAGATGCTCAATACTGAAAGAGCCCTTAGTATGCACCTCTCCTCTCATTATAGGGATGGGGAAACTCAGGCCACAAGGGGGC
CGCCTGCCTGAGAGTTCTGTTAGAAGCAAGAACCCGAGTTTCTAGTTGAATTTCTGTGCAACTTGATCTTTCTCTGTTTCTGTGGTTCTCGGTTCAT
AACCAGCTCCTGAGTCATAGCTTCACCCTTCAAACCCTGCTCACCTGTGCCACTCAAGATGATCTAGTCTACACCAGAATCAGGTATGTCTTGGGCC
TTCCAGGGGTGAGTCATGTAGCACTTGCTAGCAATTTCAGGACATCCCCTTCCCTATGGGCCTGGCCTGCCTTTGATTCCTCCCTGAAAACCATAGT
GGTTCCTAAAGAGAGAGCATGGGCTGCCTTCTGGCTGAATTACATTGATCCCTGATGTCCCCAGGGGAGGGATGGTATGCCGGATTTGGAGAGCCAT
CTTGGCACAGCGAGCAGGAGCCACATCGGTCACCCCAGTACCCCGGGACGCTGAGTGATGACATCAAAGGACACAGATACGGATGGATGAACAGAGA
AGACACAAACAGCTTTGGACACACCTGTCCTAGAACCCAGGAGCAACTACAGGGACCAGGCCCAACCCCAATGGGTGGAACTAACTAGGGAGGCACC
CAAAGGACACTACATACAATCATAGACTATTAAACAGAACACTTGCTACCATGCTTGTCTTGGCAACTTCAGAAGGGTGTTGGGGGGAGTGAGGAGT
GTCCAAGGAGAAGCCCCTTGAGGTCTCCATGTCTGAGTGACCCTTTTTCATTTCCTGGGCTTTCAAGCTAGTGCCTTACTACCCAGACTTCATACCC
AGTTAAGCAGTCAGTAGCTCTGTAAACAGACTTTATTGATTCCGGCCATGGGTGTGCTTTAGGTGGGGGGTCCACAGAGGGGTTTGACAAGGCTATG
CCCTGGGGACCAGGTTGGTTCTGAGGGGCAGAAGGCCATCCACCCACTCACAGGGCCGCTCCAGGAGTGTCTGCCACAGCTGCTTCTCCTCCGGTGT
GGAATCCATCCAGGGCACCTGAAGCCCATAACTGCTGCCTGGATGTGGGTGAGCCAAAGTGGAAGCTACTGTTACCTTGGAGACCTCGGGTCCCAAG
AGCAACTTTCTGGTCCCAGATTATGCATTGTGCCCAAGCATCCCGAACCTCCCAGCTTGGCTCCCCTCAAGGGCCTGCTCTGCTCACCTGTACCAAG
GCTGAGGCGTGTGCCCCCCAGCTGGCGACTGGCCAGGGCCCCATGGTCCCACAGGGAGAGCTCGGCACAGGCTTGGCGTAGGTCAGCAGGCCCGAAG
CCGTCATAAACCATGGTGTGGTTGAACACAGGGCTGAGACTTCTTCGAACCACTCTTGTGCGTTGGCGGCTGGCCCGACTGTCATCAGGCAGCACTG
AGCTGCGACAGGGTAATAAAGGAGGGCTAGTCTTAGGCAATCACCCTCTGACACTCCCTCCCTGAGCCAGCCAGGCACCATGTCCTGTCCTTTCATA
TGGCCGTGTTCCCTGACCGTTCCCTGGCCCTGGCCTGAGATCAGGTCTCTAGCCCTCTTACACTAATCTTTTCTTAAGGCTTCGTACGTGAAGCTAA
CTGTTCACAGGATTCCAGAGAGAAGTCTCTGAAATGCAATTCTGCTATTTTCTGGCCCAGTAACCTTCAGTATTCCCTGCCCTCCCCTTTAGTTTCC
AAAGCCTCAACCACTCTGTAGAGTAGAATAATATTACTCTGATATAGAGTAATAGAAAGGAAGCCAGGACGGGAGCCCCAACTCAGAGCAGTCTACT
CTGTTTCACTCCTGGAGGGTGCGGTAACAACACCTGAAGCACCCAGAAACACTTTTAGAGGTTCTGTAAAAACTTACTTTGTAAACCACAGAGCTGG
GGTGTAGCTTAGTGGTAGAGCCATATATGTGTGTATGTATATATATGCATGCATGCTTAGGGTGACTGAAGCATTACGGTCAGTTCGCAGCATGTTA
AAAGACAACTACAATCACCAACCATTGACTCACAAGTTCAGAGCCAAGCCCCATAGCTTGTCCCTCCAAGAGTTCCAAGGAGGACCCTTGTGGCCTT
TGCATCATAGTGCGTGCATAGTAGGTGCACAGCCTCTGCAGACTGCAGCCTATGCTGGCTAATGTCAACTTGACACAAACTTGAGCAGTGGTTCTCA
ACCTCCCTAAGGCTGTGGTCCTTTAAGACCCCCAACCATGAAATTATTTCATTGCTTCTTTGTAACTGTAATTTTGCTACTGTTATGACTTGTACTG
TAAATATCTGATGTTTGCCACCCACAGGTTGAGAACCACTGAGCTAGAGTCCTCTGAGAGGAGGGAATAAGCTAAGCAAATGCCTCCATAAGATGGG
TCATGCCGTGGCCAAGCCTGTAGAGCATTTCCTTAAGTACTGATTGAGCAAGGCTAAGCAAGCCTTGGAGAGCAACCCATGAGGAGCCAGGCAGTCA
GCAGCCCTCCTCTGTGGCCTCTGCATCAGCTCCTGCCTTCAGGTCCCTGCCCTGTTTGAGTTGCTGCCCTGGCTTCCTTCAGTGATGGACTACAGCG
TGGAAGTGTAGAACCCTTTCCTCCCCAACCTGCTTTCTGGTCGTGGTGTTTCATCAAAATAATAGAAACCCTAACCACATAGAGCCCCTTAGTTCCC
CAACTCAGATTTTGTTGTTACACAAGGTCTCACTATGTAGCTTGGGCTGACCTGGAACTAGCAATGTAGACCAGGCTGTCCTGGAACTCAGAGACAC
ACCTGCCTTTGCCTCCCCAGTGCTGGGATTAAAAATGCACACCACCACCAGGCAGTGGTGACGTATGCCTTTAATCCTAGCACTCGGGAGGCAGAGG
CAGGTGGATTTCTGAGTTCCAGGCCAGCCTGGTCCACAGAGTGAGTTCCAGGACAGCCAAGGCTACACAGAGAAACCCTGTCTCGAAAACAAACAAA
AAAATAAAACCCAAAAACATGCCACCGGACCTAGCCTAAGCTTTTATTTTGAGACAGGGATCTTGTGTATCCCATGTAGCCTTAATGACCTTGAACT
AATCCTCCTGCCTCCAATCCTAGTTTATGTAGGTTGTTAGAGATCAACCCAGAGCCTCTTGCATGCTAACCCAGCAACTCCACCAACGGATACCCAG
CCCCAGCCCCCAACTCCAACCCCCTGCCTGGCTTATCACCTCTGCCCTGCTGATCTTGGCTGGGGGGACAGGTGGATCAGAGGTCAAGAGCAGAAGG
GACTAACACCTTAGGCGGCCTCTCACCATTGAATGTAAGTGTCCAGGGATCCTGGTCGCAGTGGTACCAGGCTCTGAGCTTCCTTTACCCAGAAGTG
TAACTCCCCACTCTGAGGCTGTCCTCCTCCTGTGGGGACACTCCAGTTCAGACTACCACCTCACAGAGTGGGTGGGTCTCCCACCTCCATGGGCCAG
TCCAGCTTAAGCTCTCTCTTGGCAGTCACTTACCCTCTGAGCCGGCAGGGACATACTTAAGGGACAGAGACAGCAGTCCACGGCTGGGAAGCTCATC
TGGAGATGGAGGAACCTGAAAAGGCGGGGAGTGGAGAGAAACAGGATTATGTGTGAGAAGGGTGTTGGGGACAGGGCCTCTACTGAGGAGCCCAATG
GCAACGTTCCCCAGAAGAGGCGCCCTGGCTGGCAAGCCTGGCCTAATGGTATCAGTATGAGGCTAAGTGAGGCTCTGCAAACCGGTCCGTTGGGTGT
TTGGTCCAGTGGGCCTGTGGGAATATCAGTGACTGCACACCAGCCCTTACCCGAGGCTGCAGGGGGAGCCAGGTAGCCTCAGAGTCCCAGTTCCACG
TGTCTAGGGGCACTTCTACTTCTCCCAGAAAGATGTTGCGACCCAGACTTTCCCGGTGCCACACGGACAGGCTCAGCACGCGGCCTGGGAGATCAGC
CTGCTGGACAGAGTGCTGGGGACAGGAGGTGAGTTTCAGAGGCATGCCCTGGCATCGCTGTGGCTCACCTGTTAGCTGGAGATGGTCTCCCAGGTTC
CTTTCTAGGATGGGCTCCTGAGGCAGGGTTAACTGGGGTCCTAGCACAAGTTGTGGGGCTTCAGATTGCAAGACCACTAGCTGTCTTGTCATTACTC
TTCTGGGGCCGTCAGGAAAATTAAGGAACCCGGCAGCCCCTGCCACTCACCCCACTGAAAATGGTACCCTGTTTATTTGCAGGGATGACAGAACCCT
TGATGCCTCACCCGCAGAGTCTCGTTGAAGATCGGGTTCAGATTCCGTTTCTTCACTGACGTCTTACGCTTGCTCTGCTTATCCGGGAGGAGGTAGC
TTTTCACGTAGCTGGGCAAGGGATGTGAAGGCTGGAGGTAAGGAAGAGCCCTTCCAGAGCTTTGTCCAGCTCTGGGGTCTTCTCTAGTCACCATCCA
TAACACAGCGTGCCCATGTGGATGTTAATTAACAGGCATCCAATTTAAGACGCTTCCCAATCATATTGTTGTGCCCATTTTGAAGAAGAGAAAACTG
AGGCCGAGGCAGTTAAGTGGTAGGCTGGGTGATGGAGCCCCGCCCCTCAGGCCCCCTGCAGCCCACCTGTGACGTAGGCACTCACGGATCGGAGCGG
CGTCGCCGGGCAGCGGCCAGGCCCTGGCACTGAATCACCTGTACTCGCAGCTCAGAGGTGCCAGGCTCGTAGTGCAGCGAGAAGAGCACGGAGCCTC
GCACCTGCACCGTGCCTGCCTCCTCTCCGGATAGGCTCATCAGGCTGCCGCTCAGCTGTGGGCGGGGCGGGTGGATGATCAGGGTCCCCATAGGGAA
GCTTCCCACTGTGGGGTTTGCCTGGGCTTTCCCAGCTCCCAGCCCCTCCCTAGCTCTTCACCGTAGAGGAATTGAGGCTGGACACAGAGGAGCTGCT
GCTGAGCATGCGGTCAAGAGAGGGGCCGAGCCCCGGGGCCTCTTCTCCATTCTCCAGGATCTTGGAGGTCGCCTTGGACTGGAGAGGGGAGAAGCGA
CAGGGGCGCCCTCTTACTTCCCTGGACCTTGCAGGGGAGTTGGGGGTAAGGGGTCATTCACCGACGGTTCCCACCCCCAGGGCAGGTACACTCTCAG
TCTGGAATCCGCTCCCGGTCTTGAGAACAGCCAGTGGCTACTCCCGCCTACCTGGGCTGGAGTTGGCTGGGACTCCTGCTCCGCCTTCTGCTCAGGA
TCCAGCTCTGGGTCCGCCTCTGCCACCTGCACACCCCCTTCGCCAGGGGCTGGAATGGGGTACAAGTTGCTGAAAGAGCCCCTCTCCTATGTGTCCC
TAAGCAAACCTGGAGGGTTCACCTGTCAGCTTCAGGCCCCAGGGGTGACCTAGAGTTGTCCCCGAAGGCCAAGTTGGGGTCCCAGGAAGAAGAGGAA
GGTGGACATGATGGTTGTGGGGGCAGTCATTAAAAATGGCCTCTGATCCCCTCAACCTCTTAGGTTCCCCAGCTTACCCTCCAGCTCACATTCCTGG
TCCTGGGGCTCCTCCTCCTGACCTTCGGAAGCCTTTGAGGGTACATATGGTGAGGACAAATCAGGCCCCTGAAGAAAGCAGAGGGTAGAGGTCACGG
TGGGGGTTAATGAGAAAGGGATCAAGGGTTATGCTGGGGTCAGAGGTCAAAGACTCCAGCCTGCTCACCTTGCTTGGTCTGAGTTAGAGTAGGGCTG
CCCTCAGGGCAGGGCTACCTACCAGCTGCAGGACCAGTCAAGCCTGTACTTCACACCCTGGCATCCCAGGCCTGTCTCTCCTGCCGGGCCTGCCTGA
CTCCTAGGCTCAGCTGGTGGGGGCCCCAACATCTGAGTCCCTGGCCTCACCCGCCTCAGTCCAACCCCACCTTTGCTGGCGACACCTCGTGTGTGGT
TGACAATGACCTTGGACTCCTGTTTCTACCTCCTGAGTGCCAGGATCTGAGGTGTTCACCACAATGCCGGGTTTATGTGGTGGCTGGGGACCAAACC
CAGAGCGTCATGCGTGTTAAGCCAGCACTCTATCAACTGAGCTACATCCTTTGTGTTTGTTTTTCAAGGCGGCGTAGGGCGGGGTTCCCTGTGTAGC
CCTGGCCTTCTGAAACTCTAGAGTTGTTAAAAATTGGAGGCTGGCCTCAAACTCACAGAGATCCACCTGTCTCTGCCTCCCAAGTGCTGGGATTAAA
GGGGCAACCTGCCCCACAACCACAGCCCTGCG1GTATCCTTATTCTTTGGTTTTGTTGTTTATTTAGACTTATTTGTTTTATGTAAGTACAGACACA
CCAAAAGAGAGCATCTGACCCCACTACAGATGGTTGTGAGCCACCATGTGGTTGCTGGGAATTGNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNTTCTTAAGGAATAACTTGGGATT
CTCCAGGTTGAGCAGCATAGTAATCACTGTTCATTAAGTGCTTATTGTGGGCCATGCCTGATAGTTTATTTTGCACATTACATGTCATGTGTGCGTG
GTATGCATGCCTGTATGCTCCCATGTATGTGGTCATACGCATGTGGAGGTCAGAGTATGAACTTGCTCATCTTCAGCTCTCTCCATCCTATATACTG
AGGCAAGGTCTCTCACATAAGCTCTAAGCTCAGTGCTTTGGTGAGTCTAGCTAGCCAGCTTACTCCAGCAATGCCCTAGCACCATTGCCGGAGTGCT
GGGACAGTGAGTGGGCCACCTTACCCCTGGCATTTATGTGGGTGCTGGGGATATGAACTCCATTTCTCAAGCCTATGCAGCAAGTGTTTTACCCACG
GAACCATCTCCCTGACCCCTTAGCTGATGTTTTAGTGGAGAAAGGGGAGGTTCAAGGTCAAAGTAACTGCCCAAGGTTACACAGCTGTTCCCAGACA
CTGCTTGTCACTCTTGAGGTCCTCTGCCCTTCTGCTCCTCTGGGGAGACGAAATATCTTCGTCTTGGAAGTCAAAAGTACTCAAGGATTAGGGGACA
TGCTCATGGCTATATCAGGTTTATATATATCTGGGAGCTGCCAGGAACCGATCAGGGTGACCTATGGGCACACTTAAAGGCTTTGACACTAGGTTCT
AAGGGCTGAGGGAGGGTCCAGAAGGGTAGTACTAAGGACAGGCAGTATCTGTTCACCAAAGTATCCTGTCCCAAGGTGGCGGCCAGAGTAGAAAAAC
CCAAAGACATGGAGGCTAGGGAGGGACCTGTCACAGGTTTGAGAGAAGTGAAGAGCTCTGAGCTAGCCAAGGTCAATGGAGACCAAAAGGTAGCACC
AAGAACCACCCAGACCCAACCACCAGCATCCAAGGACCCCGAATCCCTTCCTTCTATCTGATGGCTGTCCTGTCTCACCTGACCCGCCCTTCCTCCA
GCTGGCGCAGGTGGGCATCTCGTTTGAGGACATCAGAGATGGCCTCCTGTTCCTCCTCTGTCAGGAAGCTGAGGTCCAGCAACCCCTCAACTTCAGA
TCCTGGTCCATGTGCCATGGGGAGGGAGGGCAGGGCCCAAAGCCTCTCCTGAGATGGGTGGCCTCTCTGGGGCATCAGCTGGGGTTGTGCCCCAGCT
GGGCATATGTAGCTTCCTGTGGATAAACCAAGCGCTGATCAGTATCTCTCAAATGCACAGCACACATCCTGAAGCCAGCTGCCGGCTGTCTCCCAAG
CATATACAGGAGTAATAAGGACCAATCAAAGGAAGGCCAGGTCAAGGTGAGGTGGGGCGTAAGTGATGCACTGCAAGAGATGCACATATGCGCATGC
TCCTAAGGATGAATGACAGAGTATTGCAAGACAGTACACGCTGATACACATACCCACTGTTCTACACACATTCCAGATACCTAGACAAAGACAGTTA
CATGCACACACATCCCCATGAAGGAACTGTGCGCACACATACATCATGAAGGGACTGTGCACACATACCCAGAGTCCTATGCCCAAGAGCTGTGCAG
CACAGTAATTCTTAAACCTGCACACAGTATGCCCACTCGAGGACAAGTGTGCACTCACAAGAATAAGTCCCTGCATCTCCACAGATCTCTCTGGATT
TCCTCACACACTGTCTAACCTGCTGCATACTACTAGAAACTAAGGATAGCTCACTCCCCTCAAAGCTAGTATTTTGCACACAACATAACCTGAGTCT
TAATGAAATGCAAAAATATACCTGCATAAACATGTGGATGCATGCACAGCCCTCCCCACCGCTCATGTTTATCCCCTACCTAGCCTGCCTCTGCCCT
TGCCTCCCTCCACCCTGCCCTGGCCCATTTGGCCTTCCTGTCTGAGGCAACCTCCTCCCCAACCCTCACCCCAACTTCCTCCCCCCACTCCCATAGC
ACCCAGTTCCACTGAGGCCACCTGCCTGAGATTCCTCCCCACCAGCCCTTTCTCTAGGCGGAGCTTCTGAGCCCCTTGGGTCTGACCCCCGGGCTCC
CCAACCTCCAGACTTGGCTGGAAGCCTCAGCTGGCAAAACAACTCATGCCCAGGTACCACAGCCACTCCGGAAGGAAAACATTGCCAGCCGAGAGGG
GCATGTGGACATGGCAGGGGACCTGCACTCAGTAGTGGCCCTGGTCTGGGCCCGCTGGGCAGTCTCTCTCTTTCCCAGCCCCTCCTATGCCAGGCCC
CCAGCTGGGGTTGAAAAGGGAGGAGGGCAGCTCAGGGGCAGGTCCTGGCTTGTGCCTGTCTCCTGCCTTGGTCTCCTCCCAGCTGAGTCAGTATCTA
AGCACAGCAAGCAAGCGAGCCTCCTCTCCTACCCCACTTTCCCTCCGGGGGTTTCTGCATCTCCATCGCCATAGCCCTGGGCCCGATGCCTGCTCAC
TGACAGTGACTCTTTCTCTGTACTTGAGGTGCTGGTATCAAGCTAGCTTGGGACTTTTCTATGGCGGGGTGTATGAGCATCGCCGTGTCCTGTTGTC
CTGCTGCATTAGTAGACATGTCTGCAAGCCAGGGTAAAACATAGCATCTGTCCCTGAGTGCTGTGTTGCCTTATGATACTACAGAGTCTTACAGGAG
TGTCGCTGCTGTGGGCAGTGACTTGCTGGGAGCCAGTGCCGTGTGGGTATGTGCATTTCTTCCTGTGTGGTCAGCCAGCCCAGGCTGGCTGGCTTGT
GGTGTGTATGCTAGTACCCCGGGGGGCCACCAGATATGGGAGTGAAGGTGGAAGACATTTGGCAAAATTGCCTGGCTTTGTCTGACAGGTTGGCTTG
GTCTGGGCTGTGGGCCTATACAAGGCCAGGATCTCTCTACCTCAAGTTGTATCTCTTTCAGAGGTAGATTAAGGATCAGGTGTCCCTTGCCCTGTAC
TAACACTGTGGGACAGGGACCCTTTAGTGATAGAAAGTCTGTATGTGTATCTGGGCAGGCCAGAGTCATAATCTTGCTTACACCTCACCCCCACTCC
GCCCAAACTCCTGAGGTACCAGAATGTGGTCAGGACACCAGGTGGCAGTGCCTGTCCCTTAGACCACTGGGTGTGTTACCTGTAAGGGTCCAGTAGG
GACGACAACTTCAAAGGCGACTCCAGCTCTCCAGGAAGACAGTTCAGAGACTAGAAAGGCTGTGGGGACTAAAGCTGACTCGCCCAGCAGCTGCTGC
CCCAGTCCCGAGGAGGTGGAGGAGGAGGAGACACCTCAGCAGGGCCTGGTGCAAAACCG
MOUSE SEQUENCE - mRNA
TCTGGGTGTAGCACGCTGGCGGTTGACTCCCAGGACTCTTCTGTGACCTGTGAGCCGAGGATTACTGGGTACCCCAGATTCCCAGAACCCAGTCCTG
GATTGCAGATCTGTAAGGACCGCGAAAGATTGAGCAAAGAAGTCTTTCTGGACTTAAGAGTTCCGGAACAGGACGTCCAGCCCATCTCCGGATCCCG
ACTGGATCCACACCTTCAGCCTCGTGCCCTGTCCCGGGTTCCCCGCGCCCGGGGCCCCGCGCTTGAGCGCCCCGCATGGCGGGGCCATGTCCTAGGG
CCGGGGTTCTAGAACGGGCGGGCAGCTGCTGGCAGGACCCGCTGGCGGAGGCGCTGAGCCGGGGCCGCTCGTCTCCTGCGGTCACGGGTCGGGGCTG
CGCGCGGAGCCGGCCTCTCAGTGTGGTCTACGTGCTGACCGGGAGCCGGGGCCCGGGGTGGAGCCCGGGTCGGGAACCGAGGCAGAGCCGCTGCCGT
TGCGATGCCTGCGCGAAGCCTGCGCTCAGCTCCAGGCACACGGCCACCCCCGCACGTGCGCAGCCTACCCTTTGCGACGTGGCGCTAGGCGACACCG
CGCTGGACTCCTTCTACAACGCGGATGTAGTGGTGCTGGAGGTGAGCAGCTCCCTGGCACAGCCTTCCTTGTTCTACCACCTCGGCGTGCGTGAGAG
CTTCAGCATGACCAACAACGTGCTCCTCTGTTCCCAGGCAGAGCTCCCTGATCTGCAGGCCCTTCGTGAGGATGTTTTCCAGAAGAACTCGGATTGT
GTTGGCAGCTACACACTGATTCCTTATGTGGTGACAGCCACTGGCCGGGTCTTATGTGGCGATGCAGGCCTCCTGAGGGGCATAGCCGATGGGCTAG
TACAGGCTGGGGCGGGCACTGAAGCCCTCCTCACTCCCCTGGTGGGCCGGCTTGTCCGTCTGTTGGAGGCTACACCCACAGACTCTTGCGGCTATTT
CCGGGAGACCATTCGTCAGGATATCCGGCAGGCTCGGGAGCGATTCAGTGGGCAGCAGCTGAGGCAGGAGCTGGCTCGCCTGCAGCGGAGGCTGGAC
AGCGTGGAGCTGCTGAGCCCTGACATTGTCATGAATCTGCTGCTGTCGTACCGCGATGTCCAGGACTACTCGGCCATCATTGAGCTGGTGGAGACGC
TGCAGCTTTTGCCCACCTGTGATGTGGCTGAACAGCACAATGTCTGCTTTCACTACACATTTGCCCTCAACAGGAGGAACAGGCCTGGGGACCGGGA
GAAAGCCCTGGCTGTCCTGCTGCCATTGGTGAAGTATGAAGGTCCCGTGGCTCCGGATCTGTACTGCATGTGTGGTCGTATCTACAAGGACATGTTC
TTCACTTCTGGCTTCCAAAATGCCGGGCACCTGGAGCAGGCCTATCACTGGTACCGCAAGGCGTTTGATGTGGAGCCCAGCCTGCACTCAGGCATCA
ATGCAGCTGTTCTCCTCATTGCAGCCGGGCAGCATTTCGAAGACTCTGAGGAGCTCCGGCTGATTGGCATGAAGCTGGCCTGCCTGCTAGCCCGCAA
AGGCTGTGTAGAGAAGATGCAGTACTACTGGGATGTGGGATTCTACCTGGGAGCCCAGATTCTTGCCAACGACCCCATCCAGGTGGTGCTGGCTGCA
GAGCAACTGTACAAGCTTAATGCCCCCATTTGGTACTTGGTCTCCGTGATGGAGACCTTCCTACTGTACCAGCATTTCAGACCCACACCAGAGCCCT
CAGGAGGACCCCCGCTTCGAGCTCACTTCTGGCTCCACTTTCTGCTACAATCCTGCCAGCCTTTCAAGATGGCTCCTCCTCAGGAGGACCAGTGCCT
GGTACTGGTGCTGGAGATAAACAAGGTCCTGCTGCCTGCCAGGCTTGAGATTCAGGGAACAGACCCCATGAGCGCAGTGACCCTAAGCTTGCTGGAG
CCAGAGACTCAGGAGGATCCTTCCAGCTGGACCTTCCCAGTCACCTCCATCTGTGGGATCAGCACCTCCAAGCTGGACCAGCGCTGCTGCTTCCTGT
ACGCACTTCCTCCGGCCCAGGACGTCCAGCTGTGCTTTCCCAGTGTGGAGCGCTGCCAGCGGTTCTGTGGCCTAATTCAGGTCTTGGTGATGAATCC
AGATTCCTCCGCGCCCACTGAGGAGGCAGAAGGCGCAAGGGAGGTGCTGGAGTTTGATTATGAATACTCGGAGACCGGTGAGCGGCTGGTGCTGGGC
AGAGGCACATATGGGGTGGTGTATGCCGGCCGAGACAGGCACACGAGGGTACGAATCGCCATCAAGGAGATCCCAGAGAGAGATAGCAGATTCTCTC
AGCCTCTGCATGAAGAGATCGCTCTTCACAAACGACTTCGTCATAAGAATATTGTGCGTTATCTGGGCTCAGCCAGCCAGGGCGGCTACCTCAAGAT
CTTCATGGAGGAAGTGCCTGGAGGCAGCCTGTCCTCCTTGCTTAGGTCAGTGTGGGGACCCCTAAAGGACAATGAGAGCACTATTAGCTTCTATACA
CGTCAGATCCTACAAGGACTCAGCTACCTCCATGAGAACCGCATCGTTCACAGAGACATCAAGGGAGACAATGTTTTGATCAACACCTTCAGTGGGC
TGCTCAAGATTTCTGACTTTGGCACCTCTAAGCGGCTGGCAGGCATCACACCGTGCACGGAGACTTTCACAGGGACTCTGCAGTATATGGCCCCGGA
AATCATCGACCAGGGCCCGCGAGGATATGGGAAGGCAGCTGACATCTGGTCTCTGGGCTGCACTGTAATCGAGATGGCAACAGGTCGACCGCCCTTC
CATGAACTAGGGAGCCCTCAGGCTGCGATGTTTCAGGTGGGCATGTACAAGGTACATCCACCCGTGCCCGGTTCCCTGTCAGCTGAGGCCCAAGCCT
TCCTCCTCCGAACTTTTGAGCCAGATCCCCGCCTCCGAGCCAGTGCCCAAGAGCTGCTGGGAGACCGCTTCCTTCAGCCAGGGAAGAGGAGCCGCAG
CCCGGGCTCTCCTCGGCATACTCCCCGGCCCTCAGGCGCCCCTTCCGGTCCTTCCAGTCCTTCAGCTGACTCAGCCACACAGTCTCAGACATTCCCA
AGGCCCCAGGCACCCTCTCAGCACCCACCCAGCCCCCCAAAGCGCTGCCTTAGTTATGGGGACACCAGTCAGCTCCGTGTGCCCGAGGAGCCCGCTG
CCGAGGAACCCGCGTCCCCAGAAGAGAGTTCAGGCCTGACTCTGCTGCACCAGGAGAGCAAGCGCCGGCGCATGCTGGCTGCGGTGCTGGAACAGGA
GGTGCCCACACTAGCAGAGAATCTCCTGGAACAGGAACAGGACTCTCGACTCAGCAAGATTCATGTGGAACTTCTGCTTCGTGGCCTTGGGGCACAA
ATCCACACTCCTAACCGCCGGCAGCTGGCCCAGGAGCTGCGGACCCTGCAAGCTCAGCTGCGGGCCCAGAGCCTGGGCCCTGCGCTTTTGAAGGGGC
CGCTCTTCGCCTTTCCAGACGCGGTGAAGCAGATCCTCCGCAGACGCCAGATCCGCCCACACTGGATGTTCGTGTTGGACTCGCTGCTCAGCCGTGC
AGTCCGGGCGGCGCTGGCGGTGCTGGACGCGGAGTCGGAGAAGAAAGCGGTCTTACCGAGGTCAGAAGAGTCGAGTAAAGAGGAGTCCCAGCAGAAA
CCTCAGGAGAGCCAGGCGCTGCAGAGCCAGCTCCCACCAGAGCAGGGACCCCCATCGTTGATGGTGGAGTTGGGCCTTTTGCGAGCCGAGACTGACA
GGCTTCGGGACCTTCTGGCTGAGAAGGAACGTGAGTGCCAGGCGCTGGTGCAACAGGCCCTGCATCGGGTGCATGCAGAGACCAGGAAGTATGCCCC
AGCTTCCGAGACCCCAGCCACTCTCCCAAAGGACCAGAACCTGGTGCGGTGGCTACAGGAACTGAGTGTAGATCCAGCCACTATCCAAACGCTCCTG
AGTCATAGCTTCACCCTTCAAACCCTGCTCACCTGTGCCACTCAAGATGATCTAGTCTACACCAGAATCAGGGGAGGGATGGTATGCCGGATTTGGA
GAGCCATCTTGGCACAGCGAGCAGGAGCCACATCGGTCACCCCAGTACCCCGGGACGCTGAGTGATGACATCAAAGGACACAGATACGGATGGATGA
ACAGAGAAGACACAAACAGCTTTGGACACACCTGTCCTAGAACCCAGGAGCAACTACAGGGACCAGGCCCAACCCCAATGGGTGGAACTAACTAGGC
AGGCACCCAAAGGACACTACATACAATCATAGACTATTAAACAGAACACTTGCTACC
MOUSE SEQUENCE - CODING
GCGGGGCCATGTCCTAGGGCCGGGGTTCTAGAACGGGCGGGCAGCTGCTGGCAGGACCCGCTGGCGGAGGCGCTGAGCCGGGGCCGCTCGTCTCCTG
CGGTCACGGGTCGGGGCTGCGCGCGGAGCCGGCCTCTCAGTGTGGTCTACGTGCTGACCGGGAGCCGGGGCCCGGGGTGGAGCCCGGGTCGGGAACC
GAGGCAGAGCCGCTGCCGTTGCGATGCCTGCGCGAAGCCTGCGCTCAGCTCCAGGCACACGGCCACCCCCGCACGTGCGCAGCCTACCCTTTGCGAC
GTGGCGCTAGGCGACACCGCGCTGGACTCCTTCTACAACGCGGATGTAGTGGTGCTGGAGGTGAGCAGCTCCCTGGCACAGCCTTCCTTGTTCTACC
ACCTCGGCGTGCGTGAGAGCTTCAGCATGACCAACAACGTGCTCCTCTGTTCCCAGGCAGAGCTCCCTGATCTGCAGGCCCTTCGTGAGGATGTTTT
CCAGAAGAACTCGGATTGTGTTGGCAGCTACACACTGATTCCTTATGTGGTGACAGCCACTGGCCGGGTCTTATGTGGCGATGCAGGCCTCCTGAGG
GGCATAGCCGATGGGCTAGTACAGGCTGGGGCGGGCACTGAAGCCCTCCTCACTCCCCTGGTGGGCCGGCTTGTCCGTCTGTTGGAGGCTACACCCA
CAGACTCTTGCGGCTATTTCCGGGAGACCATTCGTCAGGATATCCGGCAGGCTCGGGAGCGATTCAGTGGGCAGCAGCTGAGGCAGGAGCTGGCTCG
CCTGCAGCGGAGGCTGGACAGCGTGGAGCTGCTGAGCCCTGACATTGTCATGAATCTGCTGCTGTCGTACCGCGATGTCCAGGACTACTCGGCCATC
ATTGAGCTGGTGGAGACGCTGCAGCTTTTGCCCACCTGTGATGTGGCTGAACAGCACAATGTCTGCTTTCACTACACATTTGCCCTCAACAGGAGGA
ACAGGCCTGGGGACCGGGAGAAAGCCCTGGCTGTCCTGCTGCCATTGGTGAAGTATGAAGGTCCCGTGGCTCCGGATCTGTACTGCATGTGTGGTCG
TATCTACAAGGACATGTTCTTCACTTCTGGCTTCCAAAATGCCGGGCACCTGGAGCAGGCCTATCACTGGTACCGCAAGGCGTTTGATGTGGAGCCC
AGCCTGCACTCAGGCATCAATGCAGCTGITCTCCTCATTGCAGCCGGGCAGCATTTCGAACACTCTGAGGAGCTCCGGCTGATTGGCATGAAGCTGG
CCTGCCTGCTAGCCCGCAAAGGCTGTGTAGAGAAGATGCAGTACTACTGGGATGTGGGATTCTACCTGGGAGCCCAGATTCTTGCCAACGACCCCAT
CCAGGTGGTGCTGGCTGCAGAGCAACTGTACAAGCTTAATGCCCCCATTTGGTACTTGGTCTCCGTGATGGAGACCTTCCTACTGTACCAGCATTTC
AGACCCACACCAGAGCCCTCAGGAGGACCCCCGCTTCGAGCTCACTTCTGGCTCCACTTTCTGCTACAATCCTGCCAGCCTTTCAAGATGGCTCCTC
CTCAGGAGGACCAGTGCCTGGTACTGGTGCTGGAGATAAACAAGGTCCTGCTGCCTGCCAGGCTTGAGATTCAGGGAACAGACCCCATGAGCGCAGT
GACCCTAAGCTTGCTGGAGCCAGAGACTCAGGAGGATCCTTCCAGCTGGACCTTCCCAGTCACCTCCATCTGTGGGATCAGCACCTCCAAGCTGGAC
CAGCGCTGCTGCTTCCTGTACGCACTTCCTCCGGCCCAGGACGTCCAGCTGTGCTTTCCCAGTGTGGAGCGCTGCCAGCGGTTCTGTGGCCTAATTC
AGGTCTTGGTGATGAATCCAGATTCCTCCGCGCCCACTGAGGAGGCAGAAGGCGCAAGGGAGGTGCTGGAGTTTGATTATGAATACTCGGAGACCGG
TGAGCGGCTGGTGCTGGGCAGAGGCACATATGGGGTGGTGTATGCCGGCCGAGACAGGCACACGAGGGTACGAATCGCCATCAAGGAGATCCCAGAG
AGAGATAGCAGATTCTCTCAGCCTCTGCATGAAGAGATCGCTCTTCACAAACGACTTCGTCATAAGAATATTGIGCGTTATCTGGGCTCAGCCAGCC
AGGGCGGCTACCTCAAGATCTTCATGGAGGAAGTGCCTGGAGGCAGCCTGTCCTCCTTGCTTAGGTCAGTGTGGGGACCCCTAAAGGACAATGAGAG
CACTATTAGCTTCTATACACGTCAGATCCTACAAGGACTCAGCTACCTCCATGAGAACCGCATCGTTCACAGAGACATCAAGGGAGACAATGTTTTG
ATCAACACCTTCAGTGGGCTGCTCAAGATTTCTGACTTTGGCACCTCTAAGCGGCTGGCAGGCATCACACCGTGCACGGAGACTTTCACAGGGACTC
TGCAGTATATGGCCCCGGAAATCATCGACCAGGGCCCGCGAGGATATGGGAAGGCAGCTGACATCTGGTCTCTGGGCTGCACTGTAATCGAGATGGC
AACAGGTCGACCGCCCTTCCATGAACTAGGGAGCCCTCAGGCTGCGATGTTTCAGGTGGGCATGTACAAGGTACATCCACCCGTGCCCGGTTCCCTG
TCAGCTGAGGCCCAAGCCTTCCTCCTCCGAACTTTTGAGCCAGATCCCCGCCTCCGAGCCAGTGCCCAAGAGCTGCTGGGAGACCCCTTCCTTCAGC
CAGGGAAGAGGAGCCGCAGCCCGGGCTCTCCTCGGCATACTCCCCGGCCCTCAGGCGCCCCTTCCGGTCCTTCCAGTCCTTCAGCTGACTCAGCCAC
ACAGTCTCAGACATTCCCAAGGCCCCAGGCACCCTCTCAGCACCCACCCAGCCCCCCAAAGCGCTGCCTTAGTTATGGGGACACCAGTCAGCTCCGT
GTGCCCGAGGAGCCCGCTGCCGAGGAACCCGCGTCCCCAGAAGAGAGTTCAGGCCTGAGTCTGCTGCACCAGGAGAGCAAGCGCCGGCGCATGCTGG
CTGCGGTGCTGGAACAGGAGGTGCCCACACTAGCAGAGAATCTCCTGGAACAGGAACAGGACTCTCGACTCAGCAAGATTCATGTGGAACTTCTGCT
TCGTGGCCTTGGGGCACAAATCCACACTCCTAACCGCCGGCAGCTGGCCCAGGAGCTGCGGACCCTGCAAGCTCAGCTGCGGGCCCAGAGCCTGGGC
CCTGCGCTTTTGAAGGGGCCGCTCTTCGCCTTTCCAGACGCGGTGAAGCAGATCCTCCGCAGACGCCAGATCCGCCCACACTGGATGTTCGTGTTGG
ACTCGCTGCTCAGCCGTGCAGTCCGGGCGGCGCTGGCGGTGCTGGACGCGGAGTCGGAGAAGAAAGCGGTCTTACCGAGGTCAGAAGAGTCGAGTAA
AGAGGAGTCCCAGCAGAAACCTCAGGAGAGCCAGGCGCTGCAGAGCCAGCTCCCACCAGAGCAGGGACCCCCATCGTTGATGGTGGAGTTGGGCCTT
TTGCGAGCCGAGACTGACAGGCTTCGGGACCTTCTGGCTGAGAAGGAACGTGAGTGCCAGGCGCTGGTGCAACAGGCCCTGCATCGGGTGCATGCAG
AGACCAGGAAGTATGCCCCAGCTTCCGAGACCCCAGCCACTCTCCCAAAGGACCAGAACCTGGTGCGGTGGCTACAGGAACTGAGTGTAGATCCAGC
CACTATCCAAACGCTCCTGAGTCATAGCTTCACCCTTCAAACCCTGCTCACCTGTGCCACTCAAGATGATCTAGTCTACACCAGAATCAGGGGAGGG
ATGGTATGCCGGATTTGGAGAGCCATCTTGGCACAGCGAGCAGGAGCCACATCGGTCACCCCAGTACCCCGGGACGCTGAGTGA
HUMAN SEQUENCE - GENOMIC
ACCACCAGGCAAGATGGGCCCCAAGGGTGAGCCAGGTGAGTAAGTGGGGCTGGAGGCCTCCCCGCTTTCTTTGCCTCTGTCCCTGGACTCTAGGGTG
GGATCTCTGCTTGGCTGCTGTGCCTATGATGGCTCTCTCACAGGGGAATGGATGGCTGGGAGGAGTGGGAAGGATTAGGAGAAGGGCCTGGCTACCC
TATGATGGCCCTCCTATTGGGGATGGAGGGCTGGGAGGAGTGGGGAGGATTAGGAGAAGGGCCTGGGTACCCTATGAAGGCCCTCCTACAGGGTTTG
GAAGGCTGGGAGGAGTGGAGAGGATTAGGAGAAGGGCCTGGGTACCCTATGATGGCCCTCCTACAGGGTATGGAAGGCTGGGAGGAGTGGGAAGGAT
TCGGAAAAGGGCCTGGGTACCGGCTCCACTGGTGGCTCAGCGCTGGGGTGCTGAGACATCAGAGCCAACACTCATCCCTTCCCTGGGGCAGGAGATC
CAGTGAACCTGCTCCGGTGCCAGGAAGGTGAGGCAGCCCTTGCAGGAGCCAGGAACTTGGGATCACATGGTGTTGGGTGTTGGCAGACCCCCAAGTG
TGGCCAAGATCCTCCCCACAGCCTCCTCAGAACCCTTCTTCAGGGACCTGAAGGAGCCAAGAGGGTCCCTAGGGTCCCACACTTGTTCCAGTGCCTG
GGGGTGGGGGTCAGGGAATAACCTGGGCACCACCCCAGTCCATCTCCCCTTTCCCAGGCCCCAGAAACTGCCGGGAGCTGTTGAGCCAGGGCGCCAC
CTTGAGCGGCTGGTACCATCTGTGCCTACCTGAGGGCAGGGCCCTCCCAGTCTTTTGTGACATGGACACCGAGGGGGGCGGCTGGCTGGTGAGTGTC
TGAGAAATGGGCTAACAGGCCAGGCCCACTGGTCACTTCTCCCCCTTGGCCCCTAGGGGAGCCAGAACCCACCAGAGCAAGGCATTTCCTAGAGGAA
TTCACTGACAAATGTTTAGTGAGCCCCCAATGGGACCTGAAGAAGTATGAGGGCTTCACAGAGGAGGTGCCATTTGAAAAGACCTTAAGTTGAGTGC
AGTAGCTCACGCCTGTAATCCCAGCTACTTGGGAGGCTGAGGCAAGAGAATTGCCTGAACCCAGGAGGTGGAGGTTGCAGTGAGCCGAGTTCTTGCC
ATTGCACTCCAGCCTGGGTGAAAAGAGTGAAACTCTGTCTCAAAAAAAAAAAAAAAAAAAAAAAAAATAGGGTAGGGAGGGGCATTCCAGCTAGAAG
TAAAGATAAACTCAGGGAGGCAGCAGAGTAAAAGGTTTGCAGGGAACGCGTGAGTGAGCTGAGAGCAGGCGTGGGTGTCGGCTAAAGTCTGCCGGCA
CTGACGGAACATGGATTGAGGACCTCTGTAAAGGCCAGTTCATGGAAGGCACTAAATGATCAACTTAAGAAGTGTGCAGCGCGCCCAGACGCGGTGG
TTGGCTGGACGTGGTGGCTCACGCCTATGATCCCAGCACTTTGGGAGGCCAAGGCGGGCAGATCACCTGAGGTCAAGAGTTCGAGATCAGCCTGGCC
AACATGGTGAAACCCCATCTTTACTAAAAATATAAAAATTAGCCAGGCGTGATGGTGGGCGCCTGTAATCCAAGTTATTCAGGAGGCTGAGCTGGGA
GAATTGCTTGAACCCCGGAGGTGGTGGTTGCAGTGAGCCAAGATTGAGTCACTGCACTCCAGCCTGGGCAACGGAGTGAGACTCTGTCTTAAAAAAA
AAAAAAAAAAAGAAGAGTGTGCAGGGCAATGGGTAGCCCCAGGAGAGTTTTAAGCAGGGGAGGGGCCAAGTAGAACTTCTGTTTTAAAAAGATGATT
CGGGCTACACCATGAAAGATGCACTGCATTGGGGGACCAGTGGAGGCAGGGACATGGTTTAGGAAGACAGCAAGGAGCCAGGCAGTAAAGACAGTGG
AGCAGGCCTGACACAGTAGCTCACACCTGTAATCCCAGCACCTTTTGGGAGGCCGAGGTGGGTGGATTGCCTGAGCTCAGGAGTTCAAGACCAGCCT
GGGCAACATGGCAAAACCCCATCTCTACTAAAAATACAAAAAATTAGCTGGGCGTGGTGGCAGGTGCCTGTAATCCCAGCTATGCAGAAAGCTAAGG
CACGAGAATTGCTTGNNNNNNNNNNNNNNNNNNNNAGCCGAGATCACACCACTGTACTCCAGCCTGGTAACAGGGGGTGAGACTCTATCTCAAAAAA
ATAAATAAATAAAATAAGACAGTGGAGCAATGGAAATAGAAGTACTTAGGTTGACAACTCTATAGGACCTCGGGCCTACTTGGCTTGGAGGGGGAGG
TGGAAGACATCAGGGTAAGGCCAGGGGTCCAGCTTGGGCTGTGGGAGTGGCCTCTCTAATGTAAAATATCTTGGTTTTTTTGTTTGTTTGTTTGTTT
TGAGACGGAGTCTTGCTCTGCTGCCCAGGCTGGAGTGCAGTGGTGTGATCTCGGCTCACTGCAACCTCCACCTCCCAGGTTCAAGCAGTTCTTGTGC
CTCAGCCTCCTGAGTAGCTGGAACTACAGGTGTGAGCCACCACACCCGGCTAATATTTGTATTTTTAGTAGAGACGGGATTTCACCATGTTGGCCAG
GCTGGTTTCAAACTCCTGACCTCAAGTGATCCACCCGCCTCGGCCTCCCAAAGTGCTGGAATTACAGGCATGAGCCACCAAGCGTGGCCAAATATCT
TGGTTCTTATAGTAAATGAAATTGGGTTATTTTGACTGACACCCAGCAGCGCTGGCCATCTCCTGTGGGTGGGGACAGTGTTTTGTCAATTCAGTCT
TCCCAGGGCTTGCAAGGCTGTGGCATGAAGTTGAGTGTCAGTAAAAGTTAACAAGGGAATGTAGAGTTCATAGAAATATTCAAAGCTAACTTGAAGG
ATCCCCATTTTATCAACAGGCCCCAATCCAAAGAGAGAGTGGGTATCCACCCACCCCTCACAGTTCCTGCACCCTCAGCTGCCAGTGCCCAGCCTGG
AGTAATAGCTGCTGTGCCCCCCCTCCCCCAGGTGTTTCAGAGGCGCCAGGATGGTTCTGTGGATTTCTTCCGCTCTTCCTCCTCCTACAGAGCAGGT
TTTGGGAACCAAGAGTCTGAATTCTGGCTGGGAAATGAGAATTTGCACCAGCTTACTCTCCAGGGTGAGTTCCCAGCAGGGTCCTGGAGTCTGGGGG
TTACTGTCTGCCATCCTGAATCCCTGCCCCACCCCCAAGAACTGAGGATTCCTCCATCCCTGCCTCCCCTTCTTTCCAGGTAACTGGGAGCTGCGGG
TAGAGCTGGAAGACTTTAATGGTAACCGTACTTTCGCCCACTATGCGACCTTCCGCCTCCTCGGTGAGGTAGACCACTACCAGCTGGCACTGGGCAA
GTTCTCAGAGGGCACTGCAGGTGAGTGAGCCTAAGGGGAGCAGAGGAAGGAGGCTGGATCCTTTCCCAGTGCTGCCACTGCCCCCATGAATGACCCA
GGGCAATCCTGTCTCCTCTGTGGGCCTTAGTTTCCGCATCTGAGAAATGGCAATCATGAAACAGGATGGTGCAACAGTGAAGAATTTGGGATCTGGA
GGCAAACAGACCTGAGTTTGAATCCATCTCTGCCATTTAGTACTTATGTGACCTTGGGCAAGTCCTACCCCTCCACTGAGCCTCAGTTTCCTCCTCT
ATAAAATGGGCAGGGAGGCCGGGCACGGTGGCTCATGCCTGTAACCCTAGCACTCTGGGAGGCCGAGGCGGGCAGATAACTTGAGGTCAGGAGTTCA
AACCCAACCTGGCCAACATGGTGAAACCCCGTCTCTACTAAAAATACAAAAAAATTAGCCGGGCGTGGTGGAGGGTGCCTGTAATCCCAGCTACTCG
GGAGGCTGAGGCAGGAGAATCACTTAAATCTGGGAGGCAGAGGTTGCAGCGAGCCAAGAGAGTGTCACTGCACTCTAGCCTGGGCGACAGAGCGAGA
CTCCATCTCAGAAAAAAAAAAAAGGCGGGGGGAGCGCAGGGAGGCAAGGAGGAAGTTTTGGGTGCCGCCTGAGGCTCTGTTGAGTGTTAGTGTTCTC
TGCTTTGGAAGAGGAACCACGGAAGTCTTCTTTCAGAGAAACAGTGACTATTGGGGAGAGATTTAAAAGAGGGAAGTGGTTATGAGGGAGTGCCTCA
GGGAACCACCTTTCCTGGCCAGGCACCTAAGAGGTTCCTCCCAGCTTTTCCTAGCAGAGTTGCTCAGACTGGATGGGAGCTGAGAGAAGACAAAGGA
GCTAGAGGGGGGCCGGGGATTCTGGAGGGGGTAAGACAGGGGGAAAAGCTGGGAGCTTTGGAGCCAGACAGTCCCGAGTTCAAGTCCTGCCGCTGCT
TCAACCAGCCGAGTAACCCTGCGCAAACAACCTGGCCCCTTGGTGTTGCTCGTGGGGCCTGATAAAATTCAACTCTTAGTTGCTCTGAGACTCCCAT
GGCATTATATCTCCAAAGGTGCCAGGCATGGTGCTGGGCACCAAGGTGCTTTCCATCCCTTCCCTGCTAGGGGATTCCCTGAGCCTCCACAGTGGGA
GGCCCTTTACCACCTATGACGCTGACCACGATTCAAGCAACAGCAACTGTGCAGTGATTGTCCACGGTGCCTGGTGGTATGCATCCTGTTACCGATC
AAATCTCAATGGTCGCTATGCAGTGTCTGAGGCTGCCGCCCACAAATATGGCATTGACTGGGCCTCAGGCCGTGGTGTGGGCCACCCCTACCGCAGG
GTTCGGATGATGCTTCGATAGGGCACTCTGGCAGCCAGTGCCCTTATCTCTCCTGTACAGCTTCCGGATCGTCAGCCACCTTGCCTTTGCCAACCAC
CTCTGCTTGCCTGTCCACATTTAAAAATAAAATCATTTTAGCCCTTTCAAGACTCTTCCAGTTACTGGGAAAAAACCCTAGAGGGCAGAGCAGGGAC
ACAAGTTTCAGCTCCATTCAGGGAAGAATTTCCTTCCAGTCAGGAAGATGGAAAGGACAGCCTCTAAAGTAGTGAGGCTTCTGTCTCTGGAGGTGTG
CAAGACAAGGCCAGAGGGGCATATAGTGGAAGGACTGGAGGAGATTCAAGCATCCATTGACAATTTCCTGAGGCTCTCTGGGTCCTGCAAGTGGATG
GCTTTGTGGCAGGAATTGCACAATTGCACAGCTTTTTTGTTTGTTTGTTTGTTTGTTTGTTTTTGAGACGCAGTCTCGCTCTGTCGCCCAGGCTGGA
GTGCAGTGGCACCATCCTGGCTCACTGCAAGCTCTGCCTCCCAGGTTCACGCCATTCTCCTGCCTCAGCCTCCCGAGTAGCTGGAACTACAGGCACC
CGTTACCACGCCCGGCGAATTTTTTGTATTTTTAGTAGAGACGGGGTTTCATCATGTTAGCCAGGATGGTCTCAATCTCCTGACCTCATGATCTGCC
CTCCTCGGCCTCCCAAAGTGCTGGGATTACAGGTGTGAGCCACTGCGCCCGGCCGCTTTTTTTTTTTTTTTTTTTTTGAAATGGAGTCTTGCTCTGT
CGCCCTGCCCAGGCTGGAGTGCAATGNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNCCCCGCAGCTGCGCAGCCTGCCCTTCGGGACGCTGGAGCTAGGCGACACCGCGGCTCTGGATGCCTTCTACAACGCGGGTGAGAGCGCTGGG
GGCGGGGCCGGATCCGGGCGGGACTCAGGGTCCTGGGAGGGGACCAAGCCGGGCTCGAAGGCTCAAGGGAGGGGCCGAGAGGGGTACCGGGTCTAGG
GGACTTAAAGTGAGCTAAAGCTGAAGGGCTTGGGACAGGGCCCGTGAAAAAAGTGGGATTTAGGGGATCTGGGGCGCGGCCGGGCTCGGGACGAGAC
AGGGATCCGGGCGCGGCTCTAGGGCTCGAGGTTGGGGCTTAGGCACCCTTCAGTCGGAGCGGTGCCCAGCCAGCGGGGCAAGGATGGGGAGGGGCTT
TGGAACTCTGGGGCGGGGCGGGGAGCGGCGCGGGGCTGTGGTGTCTCAGGGGTGGGGTTGGAGAACTCAGCCTCACCCTCAGGAGAATGTCACGCGG
ATCTCTTGAGGACAATTACTGGGAGAGGAGCTTGGACTTAGGGAAAGGGGCCGGGAGGCTGGAGTTCAGGAGTCCTAGCATACTATTTCCAAGGATC
GGGCTAGGAGAGGCAAAAAGAGAAGCGAAGGGGCCCGACGGGACTGGATACTGGGTGAGGGAGCTTAGGTTAAGTGGCCTCAGCGGCCCAGAAAGAA
TGGGCGGGGAAGGGGCGGCGAAGTGGGCGGGAAAGGATGGCTCAGGGGCGGGGCCAGAAGGAGGCTATTCCCCCGCGAGTCACTCCTCCCTCCAGCC
AGCCCGGATGCTCGGCCCCCATACTTACTTGCTTATGCTAGAGTCCAGTAGCTGTGCTTCCTGGATGTCCATGAAATCGTAGCCTAAAATGAAGGCT
GACCCGACCACTCGGACACAAAGATACAATTATCGACAGACACAAATCGCATACATGTTGACTCATGCAGGCCCTAGCAGACGCTGTCACACACACA
GATACAGTCAGACATAGTAAGCTATACATGTCACAGATATACACATACATACATTTAGGCACAGCGTTGTCTATGCACACTGATGGTAGCCCCTTTC
AAGAGCCTGTGCTGAAGACCAAAGGGAGGGGACTTCTATGTTTGCGCAACTCCCACCTCCTAAATGGCCCGTAACAGACTCAACTGGTCCACAGCCT
CCCTTCCCCCATGCCAGGTGCTCACAGGCTCTGAACCGTGGCCTGAGGGTCCCAACCCAGTGAGCCCAACTCCTCTCCTGCAACACTACAGAGATCA
GTTCTGTGAAAGTGCCAGGGGAACCCTAAAAAGGGCCGACTGAAGCCCAAAGCAGAACCAGACCCTACTGGAGTCCCACAGCAAGGCAGGTCAAGCC
AGGATGGACCCCGGCTCCTTCCCCATCAAGCTGGGCTTCAGGGATCAGCGCCACCCCCTCTGTGCCTGCAGATGTGGTGGTGCTGGAGGTGAGCAGC
TCGCTGGTACAGCCCTCCCTGTTCTACCACCTTGGTGTGCGTGAGAGCTTCAGCATGACCAACAATGTGCTCCTCTGCTCCCAGGCCGACCTCCCTG
ACCTGCAGGCCCTGCGGGTAGGTGGCCTGGGGCTGGTGGAGGCAGGCACAGGGCTGGGGTAAGGCCTACACAGAACACCAGTATCTGACTGTCTGCC
TCCTGCAGGAGGATGTTTTCCAGAAGAACTCGGTGAGCAGAATCCACCTCTCACTCAAAAGTGCCCTTTGACTTCCGCTATGACCTCTGACCTCCAC
TAATCCCCCTGGCCTCCTCAGTCACCTAACCCCCCACTGACATCCCAAGTATTCCTCTGTTTCCTGTTGCCTCCTCTCATGACGGGTGCCCCTTTCC
TGCGATTCCAATTCCCACTGATGCTGACCTTTGATGCTCTCACTCCCCTTGTCTAGCCCTGTGCTGACCTCTGACCTCACTGATTCCTGCCTCCCTC
CCACCAGGATTGCGTTGGCAGCTACACACTGATCCCCTATGTGGTGACGGCCACTGGTCGGGTGCTGTGTGGTGATGCAGGCCTTCTGCGGGGCCTG
GCTGATGGGCTGGTACAGGCTGGAGTGGGGACCGAGGCCCTGCTCACTCCCCTGGTGGGCCGGCTTGCCCGCCTGCTGGAGGCCACACCCACAGACT
CTTGGTAATGAAGGCCCCCAGGGAGGGTGCTCCAGGAGGGGGCTGGTGAGGGTACGGTGGGCTGGGGCTGAGGTACAGGCCCCACCATTCTCTCTCC
CTGCCTCCCCTACCCTGGGCATCAGTGGCTATTTCCGGGAGACCATTCGGCGGGACATCCGGCAGGCGCGGGAGCGGTTCAGTGGGCCACAGCTGCG
GCAGGAGCTGGCTCGCCTGCAGCGGAGACTGGACAGCGTGGAGCTGCTGAGCCCCGACATCATCATGAACTTGCTGCTCTCCTACCGCGATGTGCAG
GTGCGTGGTGTTCAGAGAGGCAGTAGGGCAGCTGGCATTTGAGGTCAGGCAAACCCAAGTTCAGTTCCTGGCTGGGCCACTTGCTCACTGGTGGCTT
TGGGCAAATTATCAAATGTCTCTGAGTGTTGGCCTCCTTATTTGTTAGAGAGGATGAGCATGTCGCTGCCTCCTGGGGCAGTTGTGGGGTTGGGAAC
GCCCAGCATGGGGCATACAGTAGGTTCTCCCTGTGAGGTGTGTCCGCTGGGCCTGAGGGAGGCGTGCAGAAGATGGTGGCTGGGTGGGATGTCAGGA
GTGGGACAGGACCCTGGCTCAAGGTTCCTTGGGGCCTGGAGGCCCATGATGCCAGTGCTTGCCGTTCCCTGATGCAGGACTACTCGGCCATCATTGA
GCTGGTGGAGACGCTGCAGGCCTTGCCCACCTGTGATGTGGCCGAGCAGCATAATGTCTGCTTCCACTACACTTTTGCCCTCAACCGGTGAGTGGCA
GAGCAAGGGTTAGGTCATAGCCACATAAAGGTTTCCGAAACCTAGGTCTCTGCGTGCAAAGCCATTGCTGTCTGGGATCACTGCTGTTCGGGGAATT
TGCTGACCGGGGTCACTGATGTCAATAGGTCCCTGTTGTTCTGAGTTACTGCTGCCTGGGTCGGGGTGGGGGAGGTGGTGCTGCCTGTGGTATGGTC
ACCGCTGTCCAAGATCACTGCTCCTAAGAGTCATCGCATCTAGGAATCACTTCCATCTGAGGTCACTAGAGAGGTCAGTGCTGGCCAGGGTCATTAT
CATTTTGAGGTCATTATTGTCAGGCCCCTGGAAGGGTCCCTGCTGGTTCAGTGTCTAGAGGTCCCTGGACAGAGTAGGCCAGGGCCATCAGGGAGGG
CCTGAGCCCATCTTTTCCCTAGGAGGAACAGGCCTGGGGACCGGGCGAAGGCCCTGTCTGTGCTGCTGCCGCTGGTACAGCTTGAGGGCTCTGTGGC
GCCCGATCTGTACTGCATGTGTGGCCGTATCTACAAGGACATGTTCTTCAGCTCGGGTTTCCAGGATGCTGGGCACCGGGAGCAGGCCTATCACTGG
TAAAGAGCACAGTGAGTGGCCGGTGGGCTAAGCTGTGAGATGAGGGGTGGTGCTGTGGGCATCAGTCCAGCCCTGTGCCCCCTACCCCCACCCCAGG
TATCGCAAGGCTTTTGACGTAGAGCCCAGCCTTCACTCAGGCATCAATGCAGCTGTGCTCCTCATTGCTGCCGGGCAGCACTTTGAGGATTCCAAAG
AGCTCCGGCTAATAGGTGAGCACAGATCCTGGCTCTTGTCTCGTCACCCACAGGGGTTCCGAGGGGCCTGGGAAGAGGGCAGGGAGAGAGCACACTG
GGCAAGAGCTAGGATGAGCTCATCCATTTTGGACTGTCCTGCCCATACCCACACCTGGGTCCTCATACCTGTTCTGGGCCCTCAGGAGCCCTCAAAC
CCACAAATGTTCCCAAACTCACATCTGCGTGTCCATTACATCCTGAATTCCACATATACCTGTGCCTGAGCTCCTGTTCCCACCCGAGATCTTGCCA
TACTCAATGCCATACTCATATCTATGTTCATGTCCACACTCGGGCCCACAGAAATGGTGGGCCTCACTCACCCCTGCACCCACACTCACTGGAGCCC
ATGCCTCTCTCCCCCAGGCATGAAGCTGGGCTGCCTGCTGGCCCGCAAAGGCTGCGTGGAGAAGATGCAGTATTACTGGGATGTGGGTTTCTACCTG
GGAGCCCAGATCCTCGCCAATGACCCCACCCAGGTGGTGCTGGCTGCAGAGCAGCTGTATAAGCTCAATGCCCCCATATGGTAGGTGGCCCCCTCTG
CATTCTTGGCCTGACCTGGGCTGTCAGCTCCTGCACCTATGTCCATTGGCATGCTAGACTGAACCCTGAGCCAATGACCTGGCTGCTTTTAGACCAG
TGCCCGTGGCACCCCATGCCAACGTTCTAGCTATATCCAGGCCAGTTTCCTCTCCCTGGCAGTGCTGGTTGGTGCCTGGGCTGAACTGAGGATGTGG
CTTTGCATGCAGGTACCTGGTGTCCGTGATGGAGACCTTCCTGCTCTACCAGCACTTCAGGCCCACGCCAGAGCCCCCTGGAGGGCCACCACGCCGT
GCCCACTTCTGGCTCCACTTCTTGCTACAGTCCTGCCAACCATTCAAGACAGCCTGTGCCCAGGGCGACCAGTGCTTGGTGAGGCCTGTAGGGGTGG
TGGACCCGGGAAAGGGGTAAGCCCCTTCTGACTGACCCCACTCCCCAACTGCCTGCAGGTGCTGGTCCTGGAGATGAACAAGGTGCTGCTGCCTGCA
AAGCTCGAGGTTCGGGGTACTGACCCAGTAAGCACAGTGACCCTGAGCCTGCTGGAGCCTGAGACCCAGGTGAAGTCGCTGCTGAGGCCCCTCACTC
ACCTTTGCTAGGACCTTTTCTGTGGAGCCTTTCTCCCATCCTCAGACCCCTCTCCTGTCACCAGCACTCCCCATCACACCCCATTTCTTCTTGCAGG
ACATTCCCTCCAGCTGGACCTTCCCAGTCGCCTCCATATGCGGAGTCAGGTGATAGGCGGGACCTGGGGTGACTGGAAAGACGGGAGGTGAGGGTCG
CTGCCACCCAGCCTGACGCTTACTGGGTTCTTCATCCCTTCCCAGCGCCTCAAAGCGCGACGAGCGCTGCTGCTTCCTCTATGCACTCCCCCCGGCT
CAGGACGTCCAGCTGTGCTTCCCCAGCGTAGGGCACTGCCAGTGGTGCGCAGCCTTTCATGGTCGGGGTACTCTGAGGGCCAGGATGCCCGGGGGGG
AGTGGGGGAAAGAAAGGGACGACGTCCGTTGGATGCTCCGGCTTAGGTGGGGACCAAGATGCCCCTCCCGCGGGTCTGACCTCCCCCACCTTCAGGT
TCTGCGGCCTGATCCAGGCCTGGGTGACGAACCCGGATTCCACGGCGCCCGCGGAGGAGGCGGAGGGCGCGGGGGAGATGTTGGAGGTGAGGACGCG
GGACTGGGAGCTCGCGGAGGGCGAGGGGCCGCGGGCCCTGCTGAACTCCCATCTCACCCCGCGCCCGGCAGTTTGATTATGAGTACACGGAGACGGG
CGAGCGGCTGGTGCTGGGCAAGGGCACGTATGGGGTGGTGTACGCGGGCCGCGATCGCCACACGAGGGTCCGCATCGCCATCAAGGAGATCCCGGAG
CGGGACAGCAGGTGCGGTGCGTGGCAGCGCGGGTGGGGCTGAGGGCTCGCGAGGGCCGAGTGAGCCGGGCCACGAGCGGAGCGGCTCCTGTGGGCGG
GGCCTAGTGGGCGGGCTCGTAGGACCTGAGAGTTCGAGCCCTGTGGACTGGGCCCGTGGGCGGAACAGCCAGGACCGTGCGGGCGGGATTGAGGTCT
GGAGGGTCCTGTGGGCAGGGTAGTAAGGGTGGGGAAGGTCGGAGAGGGCTTGTGGGCGGGGCGGGTCCTTGGGGCGGGGCCAGGCTGGCGTGGGATG
GGCGGGGCTTCTCTGCCCACCTCGGTCCCTTTCCTCTCTCCCTTACCTCCCCTCAGGTTCTCTCAGCCCCTGCATGAAGAGATCGCTCTTCACAGAC
GCCTGCGCCACAAGAACATAGTGCGCTATCTGGGCTCAGCTAGCCAGGGCGGCTACCTTAAGATCTTCATGGAGGAAGTGCCTGGAGGTACCTGCCC
TGTGTGGGATGGGAATGAAACCGAGGTGTGAAGCATGGGACCGAGGCAGGCAGGGGTAGAGAGCCCTAGGGGGAAATGCACCCTAGTGCGTGTGGGT
GGTGTAGCCTAGAATTAGCTGAGGAGTGGCGCTGGGTGATGGAGTTGGGCTACAACATGGGTGTGTCTGGGCCTTGAGCAGAGAGATGTGGGCTGGA
GCCAGTGGGTGGGAAGAATGAACTTGGACTGTCCCCACCCAGGCAGCCTGTCCTCCTTGCTGCGGTCGGTGTGGGGACCCCTGAAGGACAACGAGAG
CACCATCAGTTTCTACACCCGCCAGATCCTGCAGGGACTTGGCTACTTGCACGACAACCACATCGTGCACAGGGACATAAAAGTAAGCCCTGGGGCT
GGCCCGCCCTCATCTGCTGAGGGTGGCGGGGAAGGAAACATGGTCCCAGACGCAGGCTGGCAGTTTAGATTAAGTGAGCAGACCTGTTGCAAAGGGT
AGGGAATTCATCAGGGAGGCTCCTTAGAGAAGGAGGGGAAAGGTTCAGCTCTGAAGCCAAACCAATTGTCAGCTCCCAGAGGGGCATATAAGTGGGA
TGGCAAGGGGAGAGTGTTGACAGAGGCAGGACCAGGCTGGGATCTCCAGGAGATGGGATCCCAGAAAGAGGGTCTAGTTCTGCTGGCAGGGGCCCAG
ACCAGTGTCCACTAATGGCTGTCAATCAGGGGGACAATGTGCTGATCAACACCTTCAGTGGGCTGCTCAAGATTTCTGACTTCGGCACCTCCAAGCG
GCTGGCAGGCATCACACCTTGCACTGAGACCTTCACAGGTAACAAGGTGTGGGGGTGGTGAGTGGGGATGCTGACCTGGGGCAGAAGATCTCTTGGA
ACTTTCCAGGGAAGGGGGTTTCTCCTAGGCATGGAGTTAGAGTCCTAATGGGGAATGAAACCCAGGGTGAGTGATTGGGCCAGGGCTGAGGTGGTGG
CATCTGGAACTGGAGCTTGGAGAAGTGATGAAATTGACTTTAGCACTTGAGGAGTGTTGATGCAGGCCAAGGTGGGAGTTGGAGGAAAAAGCAGGTG
ATGGAGTTCAGGGGGACGAGGCAATCCAGGATGTGTGCTGGAGCACTACTGTGGGTGATGGAGTCTGTGAATGTGGTGGAGCCCAGGATGAATGACG
GAAGCCTTGGCAGGCGATGGAGCCCAGGGTGGTGATGGAACTCAGGATAGATGTTTGATGACCAGGATAGGTGTTGAGGTATATTTTGGATGATAGG
TTCAGTATGGGTGCAGTCGGCCTGAGGATGACGGCATTCCCCTCCCTGCTCCAACCCCATGATGAAGCCTAGATGGGCATTGGTGCTCCTGTTTCTG
TCCTAGGAACTCTGCAGTATATGGCCCCAGAAATCATTGACCAGGGCCCACGCGGGTATGGGAAAGCAGCTGACATCTGGTCACTGGGCTGCACTGT
CATTGAGATGGCCACAGGTCGCCCCCCCTTCCACGAGCTCGGGAGCCCACAGGCTGCCATGTTTCAGGTGAGACCTCTTTGGGCCTGGCCCATGGAA
GTGGCATAGGGCCAGCCTGGGCCTTGGACACCTCTGAATGTCACCCTTGTTCACCCTCCCCCCACAGGTGGGTATGTACAAGGTCCATCCGCCAATG
CCCAGCTCTCTGTCGGCCGAGGCCCAAGCCTTTCTCCTCCGAACTTTTGAGCCAGACCCCCGCCTCCGAGCCAGCGCCCAGACACTGCTGGGGGACC
CCTTCCTGCAGCCTGGGAAAAGGAGCCGCAGCCCCAGCTCCCCACGACATGCTCCACGGCCCTCAGGTGCTTGCGGGTGGCGGGATGGGCACAGTGG
AGGGCAGAGGAGTTGTGGAGCTGTGAGGGTGGAGGAGAGCCCAGCTGATGGCTCTGTCCCTCCCTCAGATGCCCCTTCTGCCAGTCCCACTCCTTCA
GCCAACTCAACCACCCAGTCTCAGACATTCCCGTGCCCTCAGGCACCCTCTCAGCACCCACCCAGCCCCCCGAAGCGCTGCCTCAGTTATGGGGGCA
CCAGCCAGCTCCGGTGAGACCAAGGCTCCTGGGATGGGTTCCTTTTGCCTGGTTTTGCCTTTTTCTCCTCTCCTACAAAAGCCCTCCCTGGGGACCT
GGGCACCTTGACCACTTGTGAATGTGGCATTTGGTGCCTCCCCATGCCCTGTTCTGGCAACTCTAACTTGGACTTGTTGTGTCCTCTCAGTGCGGGG
TTTATCTATGTTCATGCCTGTGAGGTAGGCATAAAAGTAGGAGAGCCAGGATTCAAACCCGGCAACAGAGGTGACTGGCCGGTTGCTGCCCGTCCTC
AATCAGCAGCTCGCCCTTGTGCCCAGGGTGCCCGAGGAGCCTGCGGCCGAGGAGCCTGCGTCTCCGGAGGAGAGTTCGGGGCTGAGCCTGCTGCACC
AGGAGAGCAAGCGTCGGGCCATGCTGGCCGCAGTATTGGAGCAGGAGCTGCCAGCGCTGGCGGAGAATCTGCACCAGGAGCAGAAGCAAGAGCAGGT
GGGCGGCCCACTCTAGCGCCCCCTGGTGGTCGCAAAGGGTCAGGACACCTGTTGATGTCCCCGCCCCCTACCTCCGCGGCAGCGTCTCTTTTGGCTG
GAGCACTCGCTGACAACCCCTCTCTTCCTTCTCAGGGGGCCCGTCTGGGCAGAAACCATGTGGAAGAGCTGCTGCGCTGCCTCGGGGCACACATCCA
CACTCCCAACCGCCGGCAGCTCGCCCAGGAGCTGCGGGCGCTGCAAGGACGGCTGAGGGCCCAGGGCCTTGGGCCTGCGCTTCTGCACAGACCGCTG
TTTGCCTTCCCGGATGCGGTGAGGGCGCCTTCTGGTAGTTAGAGCTGCCCAACAACAGGTAGTGAGTTCCCCGTGCCTGGTGAGGTGCCAGGACTTG
ACGTTCCCAGATTCCCTGTCTCCTCTGTGAAGATCAGGCCTGCAGCAAAAAACCGTCTGCCCAGTCCTAGCTTCCCAGGCCTTCCCTGGACCTCAAG
CCCTGCCCACCNNNNNNNNNNNNNNNNNNNNNNNNNNNNNCCCTGCCTCTAGTTGAGGCTCAGCTGTCTCAGCGGCGCCTCCCTCCCCCTGCAGGTG
AAGCAGATCCTCCGCAAGCGCCAGATCCGTCCACACTGGGATGTTCGTTCTGGACTCACTGCTCAGCCGTGCTGTGCGGGCAGCCCTGGGTGTGCTA
GGACCGGGTAGGAGGGGAATGCCACGGGCCTCCTGCCAGCGAGGGTGTGGTGCTTGCTGGGCGCGGTGAACATCGAATGGGGCCGGTGGGGGCCAGG
CGGGACAGGGGCAGGCAGGCGCCGGTGGGGGCGTGACTAAATGGACATCTCCCATACCGGCACCGCCTGGGGCCTGCCCAGAGCCGGGCACCCTACC
CCCCGCACTTTGGGGTCTGGGGTTCCCAAACGGGCGTTGTAGCCTTGCCTGAGTTCACCGCACTGGGCTGCAGAGGTGGAGAAGGACGCGGTCTCAC
CGAGGTCAGAGGAGCTGAGTAATGAAGGGGACTCCCAGCAGAGCCCAGGCCAGCAGAGCCCGCTTCCGGTGGAGCCCGAGCAGGGCCCCGCTCCTCT
GATGGTGCAGCTGAGCCTCTTGAGGGCAGAGACTGATCGGTAAAGCCCCTTGGAATCGCTCATAGCCTCTGCCGGGCTTCAACCCACTCACCACTCA
CCGCTCCATTCTACTCTTCTGCGCCCCACAGGCTGCGCGAAATCCTGGCGGGGAAGGAACGGGAGTACCAGGCCCTGGTGCAGCGGGCTCTACAGCG
GCTGAATGAGGAAGCCCGGACCTATGTCCTGGCCCCAGAGCCTCCAAGTGAGTGGGCCTTGGCTGGAGAACATTATTGGTGGGTTCAAGGCAGCTTC
TAGCACAAGGCACCCTTGGGCTCATCACCTGACTTGGGCTCATCACCTGAGCGGGCCTTGGCGGGAGAACATTATTGGTGGGTTCGAGGCAGCTTCT
AGCACAAGGCACCCTTGGGCTCATCACCTGACTTGGGCTCATCACCTGAGTGGGCCTTGGCTGGAGAACACTATTGGTGGGTTCGAGGCAGCTTCTA
GCAGGAGGCACTCTTGGGCTCATCACCTGACTGCTTCTTGACAGCTGCTCTTTCAACGGACCAGGGCCTGGTGCAGTGGCTACAGGAACTGAATGTG
GATTCAGGCACCATCCAAATGGTGAGTCGGGGAGTGCTTGGCTATCCCTGACCTCACCAGCCAAAGCCTCCATCTCTGTCCATCTTCCTGCTCTTCT
ACTGTGCCCTCTGATGCAGAGAATCTCACTACCAGAAGAACACTTAGTATTCTCGTCCCATTGTATGGATGACAAAACTGAGGCCCAGAAGGGTGTG
ATCTGCCTCATTATTTTGGCAACAGAGCAGAGCTAGTACCTGGGTCTCGACACATCTTTCTGTAATTTCTTATTTCCCTCTTTCTGCGGGTCTTTGA
CCACATCCAGCTGTTGAACCATAGCTTCACCCTCCACACTCTGCTCACCTATGCCACTCGAGATGACCTCATCTACACCCGCATCAGGTACATCCTG
GGCCCCCCAAGTGTGAACCATGCATATGGCCTCATCCTGGCCAACTGCAGGCCTGCCTAGGTTCCTTCCTGGAAACCAAAGGGGCACCTGGGTCCCC
TAGGGACAGAGGGGGCACTGGGCAGACAGTGTGTGCAAGATAGAGTCCAGGGCCACCTTCTGGCTGAGCCACACTGAGTCCATTACCCCCAGGGGAG
GGATGGTATGCCGCATCTGGAGGGCCATCTTGGCACAGCGAGCAGGATCCACACCAGTCACCTCTGGACCCTGAGAGCTGAATGAGGGCATCATAGG
CCAGACAGGCCCCAAGGATGGATGAATGGAGAGGACAAAGGCAGCTTCTGACACACCAGCCCCAGGACCTGGGGCGACTGGAGGAAGCCAGGCGAGT
GGGGCCCAGGACTGGTTCCAGTGAGAGAAACCAACCACAGGCACCCAAGCACTACCAGACAAAGCGTATTAAACAGAACACTTTTGAACCTTTGTCC
TGGCAATTTTGGAGGTGTATGGTGGGAGGGGTATCCTGGGAAGAGCCCCCTAGGTCTTTTTTAAAAAATTTTTTTTGGCCGGGCATGGTGGTGGCTC
ATGCCTGTAATCCCAGCACTTTGGGAGGCCAAGGTGGGCAGATCACGAGGTCAGGAGATCAAGACTATCCTGGCTAACATGCTGAAACCCTGTCTCT
ACTAAAAATACAAAAAAATTAGCTGGGTGTGCTGGCGGGCACCTGTAGTCCCAGCTACTCGGGAGGCTGAGGCAGGAGAATGGTGTGAACCTGCGAG
GCGGAGCTTGCAGTGAGCCGAGATTGCGCCACTGCACTCCAGCCTGGGCAACAGAGCGAGACTTTGTCTCAAAAAAAATAAATAAATAAATGAAAAA
AAAATGTTTTTAGGCCGGGTGCGGTGGCTCATGCCTGTAATCCCCGCACTTTGGGAGGCCGAGGTGGGTGGATCATGAGGTCAGGAGTTCGAGAAGA
GCCTGACCAACATGGTGAAACCTCGTCTCTACTAAAAATGCAAAAATTAGCTAGGCGTGGTGGTGCGTGCCTATAATCCCAGCTACTCAGGTGGCTG
AGGCAGGAGAATCGCTTGAACCCAGGAGGCAGAGGTTGCAGTGAGCCGAGATCGCGCCATTGCACTCCAGAGCGAGACTCCGTCTCAAAAAAAAAAA
AAAAAAAAACCTTTTTTAGAGACAAGGTCTTACTCTGTCATCCAGGCTGGAGTGCAGTGGCATAGTCATATTCACTGCAGCCTCAAGCTTCTGGATT
CAAGCGATCCTTCCTCCTCAGCCTCCTGAGTAGCTGGGACCACAGCTGTGCACCACCCACACCCACCTAATTTTTAAACTTTTTTTTTTGTAGAAAT
AGCATCTCGCTTTGTGGCATAGCTCACTGCAGCCTTGAACTCCTGGGCACAAGCAATCTGCCTGCCTCAGCCTCCTGAGTAGCTGGGACTACAGGCA
TGTGACACCACCACCTGCAGCTAATTTTTAAAATTTTGTCGAGATGGGGTCTCATTATGTTGCTCAGACTGGTCTTGAACTCTTGGGCTAAAGCAAT
CCTTCTGCCTCAGCCTCCCAAAGCATTGGGATTACAGGCGTGAACCACTGTGCCTGGTTTCTACTGAAGTCTTTGTGGCAGAGTCTGAAGTTCCTTA
ATCTGGCCCCTCTTACTTCCCTGGCTCTTTCCTACAATCACCTTTCTTCCTCCCTGCCCCAGCAGACACCACCCTTTTATTGCTCTTAGAATAGACT
TTATTGACTTTAGCCAAGGGCAGGCCCTGAGATGGGGGTCCAGAGAGAGAGGCTTGGTGGGGCTACGTCCTGGGGGCCAGGTTGGTTCTGAGGGGTA
GAAGGCCATCCACCCACTCGCACGGCTGCTCCAGGAGGGCTTGCCACAGCTGCTTCTCCTCAGGTGTGGAATCCATCCAGGGCACCTGCAGCCCATA
GCTGCTGCCTGGATGTGGGTGGGCAGGGGTTGAGGGCATGATCACACTGGACACCTTGGGGCCCCCAAACACCTGCCTGGTCCCAGATTATGCACTG
TCCCCCGTTACCACCCTCAGTTCTCAGTAGATATCAAAGCTCAGGAAAGGACAGAGAGGTATCCAGGGCCACACACTGAGCCCCCATCTCCCAGGGC
CTGTCCCAGGGCCTGTCTCAGGGTGCTGTCCCACTTACCGGTGCCCAGGCTGAGGCGTGTGCCCCCCAGCTGGCGGTTGGCCAGGGCCCCATGGTCC
CAGAGGGAGAGCTCGGCACAAGCCTGGCGCAGGTCAGCAGGCCCAAAGCCATCGTACACCATGGTGTGATTGAACACAGGGCTGAGGCTGCGTCGCA
CAACCCTTGTACGCTGGCGGCTGGCCCGGCTGTCATCAGGCAGCACGAAGCTGTGCGGGAGGTGGCCTGGCATCAGGTGACCCCCCTCACACTCCCT
AGAGTCATCCAGTCACCACGTCCCACTCATTCTCCCTCACATATGTCCATGTCCCCTGGCTCCTGCCTGCCTGGCCTTAGACCCTCACTGCCCTGGG
ACCTTTGCACCAGCCTCCTCTCAGGTCTCCTGGCTTTAAAAGTTACAGCTCCTGGCCGGGCGAGGTTGCTCACGCCTGTAATCCCAGCACTTTGGGA
GGCCGAGGTGGGTGGATCACCTGAGGTCAGAAGTTCGAGACCAGCCTGGCCAACATGGTGAAACCCCATCTCTACTAAAAATACAAAAATTAGCTGG
GCATGGTGGCATGCACCTGTAATCCCAGCTACTTGGGAGGCTGAGGCAGGAGAATCGTTGGAGCCCAGGAGACTGCGGCTGCAGTGAGCTGAAATCA
CACCACTGCACTCCAGCCTAGGCAACAGAGTGAGACTGCATCTTAAAAAAATTAATAATAGCCGGGCGCGGTGGCTCATGCCTGTAATCCCAGCACT
TTGGGAGGCCGAGGCGGGCGGATCACGAGGTCAGGAGATTGAGACCATCCTGGTTAACATGGTGAAACCCCGTCTCTACTAAAAATACAAAAAAATT
AGCTGGGCGTGGTAGCGGGCGCCTGTAGTCCCAGCTACTCAGGAGGCTGAGGCAGGAGAATGGTGTGAACCCAGGAGGCGGAGCTTGCAGTGCACCA
AGATGGGGCCACTGCACTCCAGCCTGGGCAACAGAGCGAGANNNNNNNNNNNNNNNNNNNNAATAATAATGTTACAACTCCTATTCACAGGGACTGC
AGAGCAATGTCTCTGAAATGCAGTTCTGCTATTCTCGCTGGCTCAGTGACTTTTATTATCTCCCTGTTGCTCCACCACCACCCCTCCTCAAGCAGTT
TCCAAGGTTTTTTTTTATTTTTTGAGATCGAGTCTCGCTCTGTTTTGCCCAGGTTGGAGTGCAGTGGCGTGATCTTGGTTCACTGCAACCTTCTGGG
TTCGAGTGATTCTCCCGCCTCAGTCTCTCAAGTAGCTGGGATTACAGGCACCCACCACCACGCCCGGATAATTTCTTGTACTTTTGATAGAGATGGG
GTTTCACCATGTTGGTCAGGCTGGTCTCAAACTCCTGACCTCAGGTGATCCACCCGCCTCAGCCTCTTAAAGTGCTGGGATTACAGGCATAAGCCAC
CGTGCCTGGCCTGTTTCCAAGGTTTTCAAAGCCTGGGAACCCCCTCTGCAAAATACAGTGTTGCTCAGAAGTACAGTACCTCTGTACTGCAGCCTCT
GGCTGCATCAGAGTGGACACCCAGAAGTGACCCGCCCACCTCCATTTCACTCTCTAACAGTGGGGGCAGCACCTGGAGGCACCCCTAGAAGTCCCCT
AGAAGCTTACACTGAACTATAAGTTCCCAGCCAAGCCCCACAGCCTGGCCTCCCAGGTTCTCTATGGAAGGACCCTTGCAGCCCTCAGCCTCAGCTT
TTCAACAAGTCCTATAGGCGCCAGTCACGCTGAAATGCAGCTCCTCAAAGGTGTCCTCAACTTCCCTGACTCCAGGCCTTTGCTCAAACCGTCCCCT
TCACCCCTGCTCAGTGGTGAAAGCCCAGGCTGCGTGATCAAAGGTTGATTAGTAAGGGTTTGTGGAGGTTGGGCACTCCACTGCAGGCAAAAGAATG
GGAAGCCCGGAGGGCCAGCACTCCTCACCATTGTACGTAAGTGTCCAGGGATCCTGCCCGCAGCGGCAGGAGGTCCCGAGCCTCCTTCACCCAGAAG
TGCAGCTCCCCGCTCGGGGGCAGTCCTGCGCCTGCGGAGAGGCTCGGCTCAGGCCCGCACACGGATGGAGTCCACCGCCCCCAAACCCGGGGACTGG
AGTCTCCCGCTTTTCAGGGCGTGTCCAGCTTGGCCTCTCCGGCTGTCACTCACCCTCGGAGCCGGCGGGGACGTACTTGAGGGACAGGGCGAGTAAC
CCGCGGCTCGGAAGGTCGTCGGGAGAGGGTGGGACCTGCGAGAGGCGGGCTTATCGTGGAGCAGGGGCTAATGGAAAGGGGCCTCAGGAGGGTGGGG
TCTGCAGACCCGGATGGGGTGCGGCTAGAAGAGACGGCGGGGCCTGCAGGGGCAGAACCTAGGGGTCCTAGCGCCCTCGGGGAAGGGGCCGTGGCCG
GCCGGGCTTCGCCTGGGGCCATGAGGTTAAGAGGTTGAGGGGAGGTTCTGTCGAAGGCGGGGTGTGAGGGGGCTGCGGGAGAGCCTGGTGGGGTAAA
TGCAGGAGACCCGGGCCGCAGGTCTCCCCACGCGGCCTGGCTGCCTCACCCGGGGCTGCAGGGGGAGCCAGGTGGGCTCAGAGCCCCAGTCCCACGT
GTCCAGGGGCACTTCAACTTCGCCCAGAAAGATGTTGCGACCCAGGCTTTCGCGGTGCCACACAGACAGGCTCAGCACGCGGCCCTGAAGCTCGGCC
TGCGGGACGGAGTACTGAGGACAGGAGGTGAGCTTTAGTGGAGCACTGCTGGTGGCCCGCGTAGATGCTGCGCGAGTTGGCTGAGGCAGGTCCTACC
GCGTTCCTACCATGATGGAATTCTGGGGAGCCCCTAAGGGCATTAATGGGGACCTGCACTATCACCACGCCCACCACGAGGCTGACAACACTGGATA
ACGAGTTAATGGGCCCCCTCCGCCGATCCTCCGGGCAATGACGTTACTGGCTGCCCTTGCCACCTAGCCCCGCAGGAAGGGGGCGTCCAGTTCATTA
ACGGGGAACCGCATCGTGGTCACAGCCTCACCCGGAGAGTCTCGTTGAAAACCGGATTCAGATTCCGTTTCTTCACCGCCGTCTTGCGCTTGCTCTG
CTTATCCGGGAGGAGGTAGCTTTTGACGTAGCTGGGGAGGTGAGAACCCGAGGTGAGGAGGGGCCCTCCCAGAGCCTTCTCCCCTGCCTGTGGGCTC
CCCCAGCCACCATCCCGAACACGTGCCAGGCCTCGTGCTAAGCGCTTTACAGGGATTGTCTCGCTTAATCGCACGCCTCCCAATAACGCTGTTGTTC
CCATTTTGAAGATGAGGACGAGGAGGCCGAGACCCTGTGAATGGTGGGCTGGGCTGTGACAGCCGCGCCCCGCTTGGCCGGCGGGGCACTCACGGGT
CCGAGCGGCGGCGCCGGGCGGCGGCCAGGCCCTGGCACTGGATCACGTGCACGCGCAGCTCGGCGGCGCCCGGCTCGTAGTGCAGCGCGAAGTGCAC
GGAGCCGCGGACCTGCACCGCCTCCGCGTCGCCTGACAGGCTCATCTGGCTGCCGCTCAGCTGCGGACGGGAGTGGGTGGGAGTCAGGGCCGCTCGC
AGGGCTCACGTCGGGGCCGCGGGCAGCGCGGAGGCTTCAGACCCGGGTCATAGGTGGCAGGCTGGGGGGCTGCAGCGGTGGGTCCCGCGGCGAGGGA
GGGGCAGGCAGGGGCTACGCATCGGAACGGGTCCACCCCCCGGCCGCCCGGGTCCCCTCCCTCCCGCCTCACCGTGGAGGAGTTAAGGCTGGACACC
GAGGAGCTGCTGCTGAGCATGCGGTCGAGAGAGGGGTCGGGCCCCGGGGCCTCCTCCCCATTCTCCAGGATCTGGGACGCGGCCTTGGTCTGCAGAG
GGGGCAAGTGACGGGGCGCCCCTTCTTTCCCCACCCCTGCCCCGAGCGCCCTGGGTCGCGGGCGGCTCGCAGCCCTGGGCAGGAGCGCTCCCCGCCC
GGAGTCCGCTCCGGATGTTGAGAGCAGCCACGGGCCACTCCCGCCTACCTGGGCTTGCTGGGGCCGCGGCTCCTGCTCTCCCCCCGACGCGGGCTCC
AGCTCCGGGTCAGCCTCCTCGGCACAGACCTGTTGGTCTCCTTGGCCAGGATCTGGGGTGGGGCACGAGATGCTAAGCGAGCCCTCTCCTGCCCTCC
CCTAAGCAAATTTGTAGGAACTGGTTTACAACCCCAGCGAGCTGCAGATTCAGGCCCCGGTGCTGCCCTGGGATCGTCCCCACCTCCGACGCGGGGT
GGGAGTGGGGGTGACGAGGGTGAACACAGGTGGTTGGGGCCCCTTAGAGCTGCCTCTGACCCGCGAAACCTCTCCGTGCTCCCCAACAGGCGCTCCC
ACTCACCTTCCTGGGCCTGGCACGCCTCCTCAGGATCTGAAGCCTTTAGGGGGACAGAAGGCGATGGGAAATCAGGTCCCTGAAGGAAGAAAGGGGT
AGAGGTCACAGTACGGGGGCTGGTGAGGAAGAGGTCAAAGGTCATATTGGGGTCAGAGGTCATAGGGCTCCAGCCAGCTACCCCAGTTTCAGGTTTA
GGGCAGCCCTCCGGGCAGGGCTGGCAGCCAGCTGCAGGAGCGGTGAGACCAGTGCTTCACAGCCCACCCCGCAGGGCTTGTGCCGCCTGACTCCCTA
CTCCCATTGGTGGGGTCATTCGTACCTGAGTCCTCCACCTCCGCCAGGCCAGGCCAGTCCCATTCCTTTCTCACTCCTTTTTTTATTTTTATTTTTT
ATATTGACCCATTGGTAGACAACCTTTTTTGCTTTTTTTTAATNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNTTCAAGCGATTCTCCTGCCTCAGCCTCCCAGTAGCTGAGATTCCAGGTTATGCA
CCATCATGCCCAGCTAATTTTGTATTTTTAGTAGAGATGGGGTTTCTCCATGTTAGTCAGGCTGGTCTTGAGCTCCTGACCTCAGGTGATCTCGGCT
TCCCAAAGTGCTGGGATTATAGGCGTGAGCTGCCACGACCGGCCTTTTTTTTTTATTTATTTTTTATAGACATGGGGGGCATTTTGCTATGTTGCCC
AGGCTGGTCTTGAACTCCTCGGCTCAAGCGATCCTCCCACATCAGCCTCCCAAAATGCTGGGATTACAGGCATGAGCCACCGTGCCTGGCCCCACTT
CACCCCTTAATAGCCAGATTCAGAAGTTCCCTTAGAGGCTGGGTCCATATACCACTCACTGTTCACCACCGTTCACCACTGTTTACCACCATTCACC
ACCGTTCCCCACCGTTCACCACTGTTCCACCCAGGAGGTGATGAGGTGCAGACACAGGAAGGAACATTTCCAGGGTCTCCCCATTCCCAGGCTGGGG
CTCTTTCCCCCCTGCCCCCTGCACATTCCACCTCCCCTCCAGGGACACTGTACTCACATGTTGGCTCATTCACTTACCTCAGTCTCCCTGAGCCTCT
CCTGAGGGGCCTCATCAATGGTGAGCCTAGGAGTAGGGAGAGGACGCTCTCAGAGGGAGGGACCCTGGTGACTGGTGGGCACCATCTCCCCCTTCCT
GAGACTCCTCCTCACCTGGGCTCTGGCCCCTCTTCCTTCTCTTTCACAGCAGCCTCAGCCTCCCTGTCGTGGCCTGGAGCCTGGTCTCCTGGGCGCA
GGTGAGAAGTTCTGTGAGCCCCCATGCTCAGGAAGCCTGGCTGTGACTCGGGAGAGGCCCTGGGAGATACCCACCCACCCCATGCCCACGGAGGGGC
ATTGCCCACTGAAAGACACACAGTACTGGGGGCAGCCAGGACTGGAGCCCTAGTGGTCCTCTCTCCCTGTCCACATACAGCCAGCGCCCTGGACGGC
ACATGGCTTGCCCTGCCCCGAGGATCTCCTCTCACCCCTGGTGCTCTTCTTCCTGCGCATAGACGCTCGGACAAGGTCAGAGCCGAAGTGGGCATTG
TGGTGCCGCTGGGAGCGTGCTTCCTGGAACCAGTCCCCTGTCAGGATCTTCAGCTGCCCAGGGTCTGCCACTGAGGCCCGGAGCTTGCTGGAGGAAG
GGGGAGGGTGTGGCTGTCATGACTCACCCAAGCAACATTCCCGCCACCTACATTGTTAACAGGCTTGGGACCAGGGGCCAGGGACCCAACAGCAAAT
CAGACTTGATCTGTGCTGTGGAGGAGCTCGGGCTGCTGGGGAGACCAAGTCCAGAAGAGACAAGTACACTGTAGTGAGGTGGATGATGTGATAGAAG
TAAGAAGAGCCAGCTCTGCCTGTGAGAATCCAGGAGGGCTTCCCAGAGGAGGTGACATTTGAATTGCCTTTATTTATTTATTTACTTAATTATTTTG
AGATGGAGTCTCCCTGCTATGCCCAGGCTGGAGTGCTGTGGCGCAATCTCAGCTCACTGCAACCTTCACCTTCCAGGCTCAAGTGATCCTCCCACCT
CAGCCTCCTGTGTAGCTGGGACTACAGGCGTGTGCCACCATGCCCAGCTAATTTTTTATATATTTTTTTGTAGAGACGGGGCTTTTCCATGTTGCCC
AGGCTGGTCTCTAACTCCTGGACTCAAGCAATCCACCTGCCTCAGCCTCCCACAATGTTGTAAGTCACTGTGCCCAGCCTTGAATTTCTTCTTAAAA
GACAAATTGGAACTCTCCAGGTAGAGAAGGATAATAATTATTATACTTTTAATTTTATTATTTTTTTTTGAGACAGGGTCTTGCTCTGTTGCCCGGG
CTGGAGTGCAGTGGCATGATCATGGCTCATTGCAGCCTGACCTCCTGGGTTCAAGCAATCCTCTCACCTCAGCTGCCTAAATAGCTGGGACTACAGG
CGCACACCACCACGCCAGGCTAATTTTTTATATTTTGTAGGAATGGGGTCTCACTGTGTTGTCCAGGCTGGTCTTGAATTCCTGGGCTCAAGCAATC
CTCCCGTCTCAGCCTCTCAAAGTGCTAGGATTACAGGCGTTGCCACTATCTCTGGCCAATTCCTCTTAAATCAGTGAAGTAGGACTCCTTAGCCCCA
TTTTACAGACGACGAGCATGGTGCTCAAGGAGGTCAGGTGAGTTGCCAAGGCTACGTAGCTAGGAAGTGTCTGTCCCCAAACATTGCTTGTAACGCC
CATGCTCTTCTACAACTGTGCTCTTCCAGGAAGAAGTACCAGCATAGGCATAGAAGTGAAGAGTGGGTTCAGGGACTGGGGAGCACCTGAGTGGCTG
GAGGGGTCACTTTGTGTCTGTGTGTCGGGTCGGAGTTATGGTGAAAGAGGAGATGGTGGGGGCTGGCAAGGACCGACCAGGGAGGCCCAGTGGACCA
TGCCTAGGAGCTTGCCCTTGATACTGGGGAGCCAGGGGAAGTTTTCAGCGGGGATATTAAGGACGGATCTGGCCAGGCACGGTGGCTCGCGTCTGTA
ATCCCAGCACTTTGGGAGGCTGAGGCGGGTGGATCATCTGAGATCAGAGGTTCAAGACCAGCTTGGCCAACATGGCGAAAACCCATCTCTACTAAAA
ATACAAAAATTAGCTGGGCATGGTGGCATGTGCCTATAATCCCAGCTATTTGGGAGGCTGTGGCAGGAGAATCGCTTGAACCCGGAGGGGTGGAGGT
TGCAGTGAGCCAAGATTGTGCCACTTCACTCCAGCCTGGGCGAAAAAATGAAACTCTGTCTCAAGAAAAAAAAAAAAAAGCAGACAGATCTGGGGAG
CACCAGCTGCAGGAGGTGGGCCCAAGAGGCAAGCCTGGAGGCAGGGAGGCTGGCGAGAAAGCTGGTGCAGTCACCCCTGGAGAAGTGATGAGCTGTG
AGCTGGCCAAGGCCAGAGGAGACTGAAGGGCAAAACCAAGAACCACGCAGACATCTGACAGGCAGTGGTGTCGGTAGGGAGCAGGATGGGGGCCACT
CAAGCCTCTTGGTGCTCCCCGGCCAGGGTCTGCCCTGCCTTACCTGACCCGCCCCTCCTCCAGCTGGCGCAGGCGGGCATCTCGTTGGAGGACGCCA
GCAATGGCCTCCTGCTCCTCCTCTGTCAGGAAGCTGAGGTCCAACAGTCCTTCAGTCTCAGGCTTTGGCCCATGCGCCATGGGGAGGGAGGGCAGAG
CCCAAAGCCCCTCTTGCGATGGGTGGCCCCTCTGGGGCATCAGCTGGGGGGCTGTGCCCCAGCTGGGCACACGGAGCTTCCTGGGGGCAGACCGAGG
GTCAG
HUMAN SEQUENCE - mRNA
GAATTCGAGGATCCGGGTACCATGGGGCTGGAGTGGGGACCGAGGCCCTGCTCACTCCCCTGGTGGGCCGGCTTGCCCGCCTGCTGGAGGCCACACC
CACAGACTCTTGTGGCTATTTCCGGGAGACCATTCGGCGGGACATCCGGCAGGCGCGGGAGCGGTTCAGTGGGCCACAGCTGCGGCAGGAGCTGGCT
CGCCTGCAGCGGAGACTGGACAGCGTGGAGCTGCTGAGCCCCGACATCATCATGAACTTGCTGCTCTCCTACCGCGATGTGCAGGACTACTCGGCCA
TCATTGAGCTGGTGGAGACGCTGCAGGCCTTGCCCACCTGTGATGTGGCCGAGCAGCATAATGTCTGCTTCCACTACACTTTTGCCCTCAACCGGAG
GAACAGGCCTGGGGACCGGGCGAAGGCCCTGTCTGTGCTGCTGCCGCTGGTACAGCTTGAGGGCTCTGTGGCGCCCGATCTGTACTGCATGTGTGGC
CGTATCTACAAGGACATGTTCTTCAGCTCGGGTTTCCAGGATGCTGGGCACCGGGAGCAGGCCTATCACTGGTATCGCAAGGCTTTTGACGTAGAGC
CCAGCCTTCACTCAGGCATCAATGCAGCTGTGCTCCTCATTGCTGCCGGGCAGCACTTTGAGGATTCCAAAGAGCTCCGGCTAATAGGCATGAAGCT
GGGCTGCCTGCTGGCCCGCAAAGGCTGCGTGGAGAAGATGCAGTATTACTGGGATGTGGGTTTCTACCTGGGAGCCCAGATCCTCGCCAATGACCCC
ACCCAGGTGGTGCTGGCTGCAGAGCAGCTGTATAAGCTCAATGCCCCCATATGGTACCTGGTGTCCGTGATGGAGACCTTCCTGCTCTACCAGCACT
TCAGGCCCACGCCAGAGCCCCCTGGAGGGCCACCACGCCGTGCCCACTTCTGGCTCCACTTCTTGCTACAGTCCTGCCAACCATTCAAGACAGCCTG
TGCCCAGGGCGACCAGTGCTTGGTGCTGGTCCTGGAGATGAACAAGGTGCTGCTGCCTGCAAAGCTCGAGGTTCGGGGTACTGACCCAGTAAGCACA
GTGACCCTGAGCCTGCTGGAGCCTGAGACCCAGGACATTCCCTCCAGCTGGACCTTCCCAGTCGCCTCCATATGCGGAGTCAGCGCCTCAAAGCGCG
ACGAGCGCTGCTGCTTCCTCTATGCACTCCCCCCGGCTCAGGACGTCCAGCTGTGCTTCCCCAGCGTAGGGCACTGCCAGTGGTTCTGCGGCCTGAT
CCAGGCCTGGGTGACGAACCCGGATTCCACGGCGCCCGCGGAGGAGGCGGAGGGCGCGGGGGAGATGTTGGAGTTTGATTATGAGTACACGGAGACG
GGCGAGCGGCTGGTGCTGGGCAAGGGCACGTATGGGGTGGTGTACGCGGGCCGCGATCGCCACACGAGGGTGCGCATCGCCATCAAGGAGATCCCGG
AGCGGGACAGCAGGTTCTCTCAGCCCCTGCATGAAGAGATCGCTCTTCACAGACGCCTGCGCCACAAGAACATAGTGCGCTATCTGGGCTCAGCTAG
CCACGGCGGCTACCTTAAGATCTTCATGGAGGAAGTGCCTGGAGGCAGCCTGTCCTCCTTGCTGCGGTCGGTGTGGGGACCCCTGAAGGACAACGAG
AGCACCATCAGTTTCTACACCCGCCAGATCCTGCAGGGACTTGGCTACTTGCACGACAACCACATCGTGCACAGGGACATAAAAGGGGACAATGTGC
TGATCAACACCTTCAGTGGGCTGCTCAAGATTTCTGACTTCGGCACCTCCAAGCGGCTGGCAGGCATCACACCTTGCACTGAGACCTTCACAGGAAC
TCTGCAGTATATGGCCCCAGAAATCATTGACCAGGGCCCACGCGGGTATGGGAAAGCAGCTGACATCTGGTCACTGGGCTGCACTGTCATTGAGATG
GCCACAGGTCGCCCCCCCTTCCACGAGCTCGGGAGCCCACAGGCTGCCATGTTTCAGGTGGGTATGTACAAGGTCCATCCGCCAATGCCCAGCTCTC
TGTCGGCCGAGGCCCAAGCCTTTCTCCTCCGAACTTTTGAGCCAGACCCCCGCCTCCGAGCCAGCGCCCAGACACTGCTGGGGGACCCCTTCCTGCA
GCCTGGGAAAAGGAGCCGCAGCCCCAGCTCCCCACGACATGCTCCACGGCCCTCAGATGCCCCTTCTGCCAGTCCCACTCCTTCAGCCAACTCAACC
ACCCAGTCTCAGACATTCCCGTGCCCTCAGGCACCCTCTCAGCACCCACCCAGCCCCCCGAAGCGCTGCCTCAGTTATGGGGGCACCAGCCAGCTCC
GGGTGCCCGAGGAGCCTGCGGCCGAGGAGCCTGCGTCTCCGGAGGAGAGTTCGGGGCTGAGCCTGCTGCACCAGGAGAGCAAGCGTCGGGCCATGCT
GGCCGCAGTATTGGAGCAGGAGCTGCCAGCGCTGGCGGAGAATCTGCACCAGGAGCAGAAGCAAGAGCAGGGGGCCCGTCTGGGCAGAAACCATGTG
GAAGAGCTGCTGCGCTGCCTCGGGGCACACATCCACACTCCCAACCGCCGGCAGCTCGCCCAGGAGCTGCGGGCGCTGCAAGGACGGCTGAGGGCCC
AGGGCCTTGGGCCTGCGCTTCTGCACAGACCGCTGTTTGCCTTCCCGGATGCGGTGAAGCAGATCCTCCGCAAGCGCCAGATCCGTCCACACTGGAT
GTTCGTTCTGGACTCACTGCTCAGCCGTGCTGTGCGGGCAGCCCTGGGTGTGCTAGGACCGGAGGTGGAGAAGGAGGCGGTCTCACCGAGGTCAGAG
GAGCTGAGTAATGAAGGGGACTCCCAGCAGAGCCCAGGCCAGCAGAGCCCGCTTCCGGTGGAGCCCGAGCAGGGCCCCGCTCCTCTGATGGTGCAGC
TGAGCCTCTTGAGGGCAGAGACTGATCGGCTGCGCGAAATCCTGGCGGGGAAGGAACGGGAGTACCAGGCCCTGGTGCAGCGGGCTCTACAGCGGCT
GAATGAGGAAGCCCGGACCTATGTCCTGGCCCCAGAGCCTCCAACTGCTCTTTCAACGGACCAGGGCCTGGTGCAGTGGCTACAGGAACTGAATGTG
GATTCAGGCACCATCCAAATGCTGTTGAACCATAGCTTCACCCTCCACACTCTGCTCACCTATGCCACTCGAGATGACCTCATCTACACCCGCATCA
GGGGAGGGATGGTATGCCGCATCTGGAGGGCCATCTTGGCACAGCGAGCAGGATCCACACCAGTCACCTCTGGACCCTGAGAGCTGAATGAGGGCAT
CATAGGCCAGACAGGCCCAAGGATGGATGAATGGAGAGGACAAAGGCAGCTTCTGACACACCAGCCCCAGGACCTGGGGCGACTGGAGGAAGCCAGG
CGAGTGGGGCCCAGGACTGGTTCCAGTGAGAGAAACCAACCACAGGCACCCAAGCACTACCAGACAAAGCGTATTAAACAGAACACTTTTGAAAAAA
AAAAAAAAAAAAAAAAAAAAAA
HUMAN SEQUENCE - CODING
ATGAACTTGCTGCTCTCCTACCGCGATGTGCAGGACTACTCGGCCATCATTGAGCTGGTGGAGACGCTGCAGGCCTTGCCCACCTGTGATGTGGCCG
AGCAGCATAATGTCTGCTTCCACTACACTTTTGCCCTCAACCGGAGGAACAGGCCTGGGGACCGGGCGAAGGCCCTGTCTGTGCTGCTGCCGCTGGT
ACAGCTTGAGGGCTCTGTGGCGCCCGATCTGTACTGCATGTGTGGCCGTATCTACAAGGACATGTTCTTCAGCTCGGGTTTCCAGGATGCTGGGCAC
CGGGAGCAGGCCTATCACTGGTATCGCAAGGCTTTTGACGTAGAGCCCAGCCTTCACTCAGGCATCAATGCAGCTGTGCTCCTCATTGCTGCCGGGC
AGCACTTTGAGGATTCCAAAGAGCTCCGGCTAATAGGCATGAAGCTGGGCTGCCTGCTGGCCCGCAAAGGCTGCGTGGAGAAGATGCAGTATTACTG
GGATGTGGGTTTCTACCTGGGAGCCCAGATCCTCGCCAATGACCCCACCCAGGTGGTGCTGGCTGCAGAGCAGCTGTATAAGCTCAATGCCCCCATA
TGGTACCTGGTGTCCGTGATGGAGACCTTCCTGCTCTACCAGCACTTCAGGCCCACGCCAGAGCCCCCTGGAGGGCCACCACGCCGTGCCCACTTCT
GGCTCCACTTCTTGCTACAGTCCTGCCAACCATTCAAGACAGCCTGTGCCCAGGGCGACCAGTGCTTGGTGCTGGTCCTGGAGATGAACAAGGTGCT
GCTGCCTGCAAAGCTCGAGGTTCGGGGTACTGACCCAGTAAGCACAGTGACCCTGAGCCTGCTGGAGCCTGAGACCCAGGACATTCCCTCCAGCTGG
ACCTTCCCAGTCGCCTCCATATGCGGAGTCAGCGCCTCAAAGCGCGACGAGCGCTGCTGCTTCCTCTATGCACTCCCCCCGGCTCAGGACGTCCAGC
TGTGCTTCCCCAGCGTAGGGCACTGCCAGTGGTTCTGCGGCCTGATCCAGGCCTGGGTGACGAACCCGGATTCCACGGCGCCCGCGGAGGAGGCGGA
GGGCGCGGGGGAGATGTTGGAGTTTGATTATGAGTACACGGAGACGGGCGAGCGGCTGGTGCTGGGCAAGGGCACGTATGGGGTGGTGTACGCGGGC
CGCGATCGCCACACGAGGGTGCGCATCGCCATCAAGGAGATCCCGGAGCGGGACAGCAGGTTCTCTCAGCCCCTGCATGAAGAGATCGCTCTTCACA
GACGCCTGCGCCACAAGAACATAGTGCGCTATCTGGGCTCAGCTAGCCAGGGCGGCTACCTTAAGATCTTCATGGAGGAAGTGCCTGGAGGCAGCCT
GTCCTCCTTGCTGCGGTCGGTGTGGGGACCCCTGAAGGACAACGAGAGCACCATCAGTTTCTACACCCGCCAGATCCTGCAGGGACTTGGCTACTTG
CACGACAACCACATCGTGCACAGGGACATAAAAGGGGACAATGTGCTGATCAACACCTTCAGTGGGCTGCTCAAGATTTCTGACTTCGGCACCTCCA
AGCGGCTGGCAGGCATCACACCTTGCACTGAGACCTTCACAGGAACTCTGCAGTATATGGCCCCAGAAATCATTGACCAGGGCCCACGCGGGTATGG
GAAAGCAGCTCACATCTGGTCACTGGGCTGCACTGTCATTGAGATGGCCACAGGTCGCCCCCCCTTCCACGAGCTCGGGAGCCCACAGGCTGCCATG
TTTCAGGTGGGTATGTACAAGGTCCATCCGCCAATGCCCAGCTCTCTGTCGGCCGAGGCCCAAGCCTTTCTCCTCCGAACTTTTGAGCCAGACCCCC
GCCTCCGAGCCAGCGCCCAGACACTGCTGGGGGACCCCTTCCTGCAGCCTGGGAAAAGGACCCGCAGCCCCAGCTCCCCACGACATGCTCCACGGCC
CTCAGATGCCCCTTCTGCCAGTCCCACTCCTTCAGCCAACTCAACCACCCAGTCTCAGACATTCCCGTGCCCTCAGGCACCCTCTCAGCACCCACCC
AGCCCCCCGAAGCGCTGCCTCAGTTATGGGGGCACCAGCCAGCTCCGGGTGCCCGAGGAGCCTGCGGCCGAGGAGCCTGCGTCTCCGGAGGAGAGTT
CGGGGCTGAGCCTGCTGCACCAGGAGAGCAAGCGTCGGGCCATGCTGGCCGCAGTATTGGAGCAGGAGCTGCCAGCGCTGGCGGAGAATCTGCACCA
GGAGCAGAAGCAAGAGCAGGGGGCCCGTCTGGGCAGAAACCATGTGGAAGAGCTGCTGCGCTGCCTCGGGGCACACATCCACACTCCCAACCGCCGG
CAGCTCGCCCAGGAGCTGCGGGCGCTGCAAGGACGGCTGAGGGCCCAGGGCCTTGGGCCTGCGCTTCTGCACAGACCGCTGTTTGCCTTCCCGGATG
CGGTGAAGCAGATCCTCCGCAAGCGCCAGATCCGTCCACACTGGATGTTCGTTCTGGACTCACTGCTCAGCCGTGCTGTGCGGGCAGCCCTGGGTGT
GCTAGGACCGGAGGTGGAGAAGGAGGCGGTCTCACCGAGGTCAGAGGAGCTGAGTAATGAAGGGGACTCCCAGCAGAGCCCAGGCCAGCAGAGCCCG
CTTCCGGTGGAGCCCGAGCAGGGCCCCGCTCCTCTGATGGTGCAGCTGAGCCTCTTGAGGGCAGAGACTGATCGGCTGCGCGAAATCCTGGCGGGGA
AGGAACGGGAGTACCAGGCCCTGGTGCAGCGGGCTCTACAGCGGCTGAATGAGGAAGCCCGGACCTATGTCCTGCCCCCAGAGCCTCCAACTGCTCT
TTCAACGGACCAGGGCCTGGTGCAGTGGCTACAGGAACTGAATGTGGATTCAGGCACCATCCAAATGCTGTTGAACCATAGCTTCACCCTCCACACT
CTGCTCACCTATGCCACTCGAGATGACCTCATCTACACCCGCATCAGGGGAGGGATGGTATGCCGCATCTGGAGGGCCATCTTGGCACAGCGAGCAG
GATCCACACCAGTCACCTCTGGACCCTGA

TABLE III

MOUSE NOMENCLATURE
ICSGNM Fosb
Celera mCG4848
HUMAN NOMENCLATURE
HGNC FOSB
Celera hCG20725
MOUSE SEQUENCE - GENOMIC
CTCACTCCGCCAGTCTGAAGCATCCGGGCTCTGTCTGTGAAACAGTCCGCGAACTGGGAGGGTCTACCCACTGGCCCCAGCACCCCTCCTCCCAAGG
CCCCCCATGCTGCTGCTTCATGTGACCAGGGCTGGGCCGAATCTCTGAGCAGACAGAGAGAAGCATGGTGTGACCGCATGAGTGGAGTGAGTTCTGG
GGACAGTGCCCAGTTGTGCGATAGACAGCAGATTCCGGAGGACCACTGTGCATGTGTGTGTGTTGTTGTATTTGGTTTGTGTTTGTGAGTATGTCTG
TGTGTGTGTGTGTGTGTGAGAGAGAGAGAGAGAGAGAGAGAGAGAGACAGACAGACAGACAGAGACAGAGACAGAGACAAAGAGAGTTGTGTGAGGA
CCACTGTGCATGTGTGTGTGTTGTGTGAATGTTTGTGAGTGTGTGTGTTGTTGTATTTGGTTTGTGTTTGTGAGTGTATCTGTGTGTGTGAGAGAGA
GACAGAGAGACAGAGAGAGAGAGAGAGAGAGAGAGACAGACAGACAGACAGACAGACAGAGAGACAGACAGAGAGAGACAGAGAGAGACAGAGAGAG
AGAGAGAGTTGTGTGAGGGCCTCCCAGGTGATCTGGGAAGCTTGTGAATGTGAAAGTGCCTGTGGGTGGCTTCACCTCTGGCAGAGGCATTCTGCCT
CCTGTATATTAACTATGTGTGCTCAGACAATCCACTCAAAGATGCTCTGGGGCTGCAGCTGATAGAGCAGCTTACTTAGTGTGCACAAGGCCGTGGG
TGCAAACACCCCAGAACTACAATCCCAGAACTCTCTCTGCGCACTGAACTCGACTTCCTCATTTGTTTCTTGTTTCTTTTTTTTGACGGAGTCTAAC
ATTGTCCGGATTGCTTCAAACTTTATAGCGGAGGAAGATCGATGGCAATTCTTCTGCCTCCACCTTCTGAGTGCTAGGATTGCAGGCATGTACCCTC
ATGCCCAGTTGAGACAGTATCAGTGATTGAACCCAGGGCCTCACGCCTCCTGGAGAAGCCCTGTACCATGGAGCTACAAGTCTAGTTCCTTTTCTTG
TCCTTAAAAAAAATAAAAAGCTGGGTCTTCTTATAGCCCAGGCTGGCTTTGTACTCCCCATCCTCCTGCCTCAGCCTCCCAAGTGCTGGGATGATAG
GCTTATGGCACGGCCATGATAGCACCTACAATTTCCAGAGTTGCCTGTCCTTTGCAGTGTACTATGCCTTGTGAGGGACAGGCTCTGAGGGTCTGCT
TGTGATCACACACCTGAGGACCTGTTTGCTGGGAGTTCTGTCCCCATTTCTTTGCCAGGCTGGAAGACACTGACACACCCACCATGGTGACAGTACC
CATGGACTTAAATGGTGTGACTCCCTCTGCTCTAATGGGAGGGTCTCAGTGCCCCGTATCTGCTGTTTCTCAAATGGGGTAGGTAATACAGAGATAG
GAGGGCTGACACAAAACATATCAAACACCAAGATAGCAAAGGCTAGAATGAGACCCTGTCTCAAGCTTTCCCATAGAGAAGGGGAGTGTATAATTGC
ACCCACTCACAAGACTGTTGGCCATAAACAAGTTTGTATGGAAAATGTTTACAAAGTGCTTGGCACATCCAGGGGTGGTGGCGCACACCTGGATTCT
CATACTTGGAGAGAAGAGCCAGGAAGATTAGGGACGGTTCCAGTCAGGCCTACATATTGAGACCTTATCTCAAAAACAAAACAACACAACACACACA
CACACACACACACACACACACACCTCAAAAACCTCTGGAAGTTCACATTTTGGGAAAGGCATCTGAATGGTAAAGTGTAGGGATTAAGAGTAGAGAC
TAGAGTCCCTCCATGGGACCGAGATGTAGAAAAGAAAGAAAAGAGAGCTTGCTAAAGCAGGCCAGCCTAAGGGCAGACTCCTGAAAGGACTTGCTCC
AAATGATTGTTGAGGGTGGAGGACTCTAAGAAACTGGAATTGTGGGAAAAACTCTCGGGGAATGACAAAAGCCGGTTTGCAGGTTGGTGAGATTAGA
AAGTAATGGCCACAATATAGGGCACAATATACAGGCTGGTCTTGAACTTGTAATCCTCCTGCCTCAACCTCCCAACTATTATAGGTATATGTCACCA
GCTGGATGCTTTTGCCTGTCTTGTTTCTTTCTGTGCCCCAGGGCTTCAGTGAATAGGGATGGCTGCCGATTAAATAACTGAATTGCTAGCAGGGTGG
GTGAGTCAGGGCTTTAATCCCAGCCTTCAGGAGGCAGAGGCACACAGATCTTTGTGAGTTCGAGGCTAACCTGGTTAACAGAGAGAGTTCCAGGACA
GTCAGAGCTACACAGAGAAACCTTGGCTCAAAAAACAACAAAATATCTGAATGGCTGATTGAATGACTGGGCAGATGGACAGATAGACAGATGGACA
TGTACGATCCACRGACATGTACGATCTTTGGGATGGTGGGGAAGGCTTGGTAGCTAACACAGGCTGGCTCAAGACCTGCTTCTGGGAACCCTTTCTT
TCCTTTGTATCCTCAGCTCTGGGTCATGAAGGGTGTGGAGTGGGCCGGGAAGGGAGTGTCAGGCTCAGTGGCTGCTTGCCTTGGATTCTGGCCCAGA
GTGTCTGGAGGAAGAAGAGGGTATCTCGGCATCTGAGTCACCTTAGAGAAACCCTAGCCACATACCTGACTTCTGACATCACACAACCAGGGCAACC
CTGGTGGCCGAGACAAGGAGACTGAATCATGGACTGAAATCAGTCACCTTGGGCATGGGCACGTGGCCGCTAGTGACCCCCTGAGACACCCCCGAGT
GGCTGTCTGGCTGTGTGGGCATGCAAACCACATCAAGGTCAGTAAAGGCTCTCCAGCCTGAGTCAGGGACAGGGTGGCCACTGCCAGAGACCACCGA
CCACAGCATCTGCAGGACAGGAAGAGAAAGTTGAGGGAGAAAAGGCAGAGGGTAGGCCCCAGTCAGGGAGAGGGAAGTGCCTGTGGGAGAAAGGGGA
AGCTGTGGCTTGCGGCCAGTAGCCTGGTGACCTGTGGCTGGGATGTGGAATGGGGTCTTAGTGTTTGCGCACAGAATGGCGAAGTTGCAAGCGAGGC
TCAGAGGACAATGGAGTCAGCAGACAATGGGCTCTTACACTTGCACACGCAGGCACAGGCGCACAAAAGCCAGCGTGGGAGCCCTACCCTCCAGCCA
CCCCAGAGCTACAGCTTGCCACACACACACAAGACTGTCCCCACAAGACTCGCTTCCTTTACCCTATTAAGTGACATCCGTAATTCTCATTGTGAAG
ATTGCAACTCTCGGTATTTTGATGATCTCATTTGATATGAATAACTTAGACATTTGCAGTGCCAGGCACTGTTGTAGACTATTTAGCTGTGCTAGGG
ATTGAACCCAGGGCTTCTTGCATTCTGGGCAAACATCCTGCCACTGAGCTTGCTCCCAGACCCACGTGCCCATGGTTTGTCCCCAGACCCCTGTTTT
ATATGTAATTCTGAGACAAGGTCTCACTGTAATGCCCAGAGACCCTTGAGCTCACTCTATTACCCAGCCTTGAACTCACTGATCCTTCTGCAGGGAT
TGTAGGCATGACTAGCTAGACACAGCTAAGTATCTGTTAGAGCGTGTATGTGTACCTTGGGGCAGACATCTGTCCAACTGTATATGTCTATACATTG
TTAGGTTTCAAACAAATGGCTGAGGTGCCTATTTAACATATCAAGAGGACCTGGCCCCCAATTTCTCCCTGCAATAACTCAGCTCCTATATGCCCTC
CTGGCTGGCATACCCCACCCGCTATGCTGAACTTCTCCAGCCCAGGGGCTGGGCTGTTCATCCCTATGTAATCCAACTATTTTAGCTCTCCCCTCTC
TTTGTACCTTAAGGCTGCACCTGGCTCCCATGGCTCCCCTCCCTCTCTCCCCACACGGCTCAGGGTTAAGTCGACTCTGGACTATCCTAGAGGTCCC
TGCCTCTGGCTATGCTCTCCCACATATCTATAATAAACTCTCCTCTGCATACCTAGGAACAGTCATGTTCCCTTCCTTTCTCTTTCTTAATTTCCTT
TTCTTTTCATTCATACATACGTTTCTTTGGATTCTGTATTCTATTCTCCCTCCTTAAAAATATACACAGCTAGCCAGGCATGGTGATGCACACCTTT
AATCCCAGCACTCGAGAGGCAGACACAGGTGGATCTCTGTGAGTTCGAGACCAGCCTAGTCTACAGATTGAGTTCCAGGCCTGTCTGGCCTATACAG
TGAGATCCTCAAAAAGAAAAATGTACACACACTACCCCCTTCAAGTCAGAAGACCCTGAGACACAAGCCCATCCATCGCCGGGCAGCATGGGAAAAG
CAGGTTGTTACAAATGCCTGGAGTCAGGTCTGACACTCACACACAAGGTCCCTGCCACCAGTTGATCCAGCAGCTACATTCGAAGGATCAGAGAGGT
CCAGATAACTGTGCTGCCCTGTCAGACCTTATGAATGAAGACCTGAATGGAGGCTGATGTCTGTAATTCCAGCACTCGGGAGGCACTCAGGAGGCAA
GGAGGATTGGAGGGAGTTAGCTGGAGGCCAGCCTATGCTATAGAGTAAGACTGCTTCAAACAAGAGAAATGAGAACTGAAAAGATTGCTCAGTGGGT
AGAGGCGCTTGCCTGCAAGTCTAAGGAACTGAGTTCAATTCCTGACATTTGCATGATGGAAGAAAAGAAACTCCTTCAAGGTCACACACACACACAC
ACACACACTCAAAGAAGTAAAAATACTTTTTAATTTAAAAGAACAGAGAAACTTGAACACATGGTCGGTCTGCGATGTGGCTCTGCAGTAATGCCTG
CCTTGGATGTATGTAGTCCTGGTTTTGACCCCACCATTATACAGACCAGGTGTGGTGATACAAACCTGTAATTCCAGCACTGGGGGCTGGAGCCAAG
ACGATGGGAGAAGTTCTAGGTGGCTCATCCTTGGCTAAAAAAACATAGCCTGAGCTACCTGACACTGGTTGCTAGTGTGAGCTATCTGAGACTCTGC
CTCACAAATAATTAAGTAAAAATATGGTAGATTCATGACGACAGCACCTACAGATTGCCTGTCCTGTGCTGGGACACAGGGCAGCTTCACACTAACC
CTGATGGGAACACCTGGGAGACGACATGCTTGTTGCAAGGCGCTGGCTCATCTGAGACCAAAGCGACTTCAGGAGCCAAGTAGACCAGAATCCGTGT
GCTTCAGACAGTGGGAAGGGCATACAGAAACCTCAGGAACTGAGGCACAGCAGGGAGATGGGAGGCTGGGACAAGGGACCACTGACAGACACCATAC
ATCATACCATCTTGTCCCTCTGAATCTCCATGGGCACCCCCTCTGGGAGGACTCCAGGACTGCAAGCAAAGGTTAACTGAGTCAGCCAATTCAACCC
TGGCCTAGGGGGTGGAGGCAGGGGGTCTATCAGCGCGAGGAGACAGGACAAGGACACTCATGTATTGCCATAGGGATGGCTCTCTTGTCAAACCATC
AGAACACCCACAAACATCCCGGCTCACAGGGGTCTGCAGGGGTAGGGCTGGGATGTGGCCCGAGACTTGTGGGAACTGAGGAATGTAGTGGCAAAAG
TTTGGCCAAGGGAAGACCAGAGGACTCTGTATGCATGAAGGCCAGGGTGATGACATTTCAGGAAAGAAAAGCACAGAATCAAGCATGGTAGGTAGTT
GGAAAGTGGAAGCGGGNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNACCACATT
TACTAAGCCCCTCCCTGCCAAAGTAAGCAATAAATTATGGCTAAGAGTCTCCCCACTCCTAAGATGGAAAGATGCCAAGCTGCAAAGGAGATCCTGA
CAACAGACCAGCTACTTGGAAGACTCAGAACCTAGCCAAGCTGCCTGCAAGAGGTTTAGACCAACTAAGACATCTGGAGAGAGCACCCTCCAATCTG
TGGAGCAGTCTTCAGGCTCTGCAGGGTGCTCCAGGCTCCCAGCTTTGTAAGCTGTCACTCATGCTGGGGCGGGCTTGGGTGACGCAGCTGTCTTTGA
GTGACTTCTCCCGTAAGTCACCCCTCAGCCATGCACACTCCTGTAAGTAACTCTAATAGAAAATTCATCGGTTCAGCAAATTGGACTTTGGTCTCTT
CTGAGCTGGCTCCCTGTCTAGGGTGAATGGACATGTGTGTGTGGCATCTCCCCTGGAAAAGTTTTCTTTCCCTAGGCTTGGCCTGGGGTCTCCCAGG
CTTTTTGCAGTTGCTGGTGGCATCCTTCTTAGTGGGTACTTAAGAATCAGGACCCTGGCCAGCGATGCAGCAGCCGTTGGCAGAGGGCTCCCCTAGC
ATGCAGGAGGCCCCTGGATTACATCCCCAGTAATAAACCAGGCCTGGCGACAATATAGAGGTGAGAGCAAGATGTTCATTGTCATCCTTGGGTACAT
AGTAAATTCTAGTCTTTGTTTTAAAAACAAACAAACAAACAAACAAGCAGGACATAACATCAGATATGCCTGGCATGGTGGAAGGTGCCTTTAACTC
CAACACTCAAGAGGTAGAGGTCATCCTGGCCTACAGAATGAATTCCAGGCCAAAGGTGGTACATAATGAGACCCCATCTCAAAAACAGACAGAGAAA
GGAGGAGGGCAAAGGGTAGAAAGAGGAGGGGATGTTCACAATAGGGGGAGGGGATGTTCACAATACGTGTATATATATTCTTTCCTTCTGGATGTGG
ATAGAACCCAGGACTTCACACATGCTAGACATTCATTCCACTTTAAGCTACATTCCCGCCCTCACATCCCCTTTTGTTTAAATCATTAATATTGATA
TTATTATTATTATTATTATTATTATTATTACTACATATGAGTACTGGTTGTTTTCTGTCTTTGCATGCATGTGGAGATCAGAGAACAATTTTTAGGA
GTCATTTTTCTTCTTCCACCGTGAGTCCCAGGGATGGGACTCAGGTTGTCAGGTATGTGTAGCAAGCTCCTTTACCTGTGAACCATCTTGCTGGGGC
TGATACCTGCTGATTGCTAAACACATGCCACCGATTATATTAATGCACTAGTATTTTATTTATTTATTTTTCGAGACAGGGTTTCTCTGTGTAGCCC
TGGCTGTCCTGGAACTCACTCTGTAGAACAAGCTGGTTTCGAACTCACAGAGATCTCCCTGCTTCTGCTGGGATTAAAGATGTGTGCTGCTGCCGCA
GCTGCCACCACCACCAGACACCACCAATTATTATTGTTCAATTATTATTGTATACACACTTTCTTTTCTGTCTCCTGAGCTAAGTGGGAAGGAGAGG
CTTCCAACACCATCAAGCCTGAAGAACTCACCGGGTGTGGGTTCCTACCTGCTCCAATTCTGCAGGTCCCCCAGATTCAGGGACCCGGAAAAGCCAC
CTATCCTTTGCTTAGCTATGAGTACTGAAGTTTTAGATCAGGGAGGGAGCACATTTTCCAAGCTCACCGAGTCACACAGAATCCGTGACAAAGCTAG
TGGGGAGGGGACTGCATCCTTACCCATCCCTACACCTGGGGATATCACAGACTCTCCAATTCCCCCTGAGCCTCAGTTTCCCCACCTAGACTGATTA
TACCCCTCTCGCCAGAATCTGCACTGGGGACCTTGCTCTTTTTCACCAGCAGGGGCGCTGCACCGGTTTGGGGAGGGTGGGGTCCCAGGGGTATAAG
CAGACCTGGGATCTGGAGTTGCACCTTCTCCAACCCGGGTCAGCAGGGGCCTTCTGAGGGAGTTTAGGCGCCTGTCAATCTCAGCCTCCGGGACAGC
GTGGAACTGCGCAGGCGCGGGCGGGTTCCGCACAGCGCAACAGCGGGCGCGCGCGGGAGCGAGGGATTCCCTCTGACGTAATTGCTAGGATACCAAA
CAAACACTGGGCCGCGCTGGCCGAGCTCCTTATATGGCTAATTGCGTCACAGGAACTCCGGGGAGGGCGGGGCGGGATCCCCTCCCGCGAAGCCCCT
CAGAACGCAGCCTTGGGGACCCCCGAGACCCCCAGGGTCACACTATGGGCAGGTGGCAGGTGCACGAGGCCCTGGAGGGCGCCACAAGGAGCTCAGT
CGGCGGGAAGGGAGAGTTTGGGAGGTTTGTGCATGGAGTTGCGGGTGACGCAAGCGCGGGGGGCGGGTCCCGGAGGCATAAATTCAGCCCGGCAGCT
CCCGGTTTCATTCATAAGACTGGAGCGGCTACGCCGGGGACACGCGGAGCCGGGGCTTGCTGGACTTGACTACTGGTGACTCTTTCTTTTTCTTCTT
CTTGGAGGCCGTGAAAAATTTATATATATATATTTATATATATTTTTAAACTGGAGAGAAAGTTTTGTGGGTTTCCTTTTTGCAAGACTCTTTCTAG
ATCGTTCTTCCGATTCGTCCGGAGGATAGACTTTCTTTCTGTGGGCTTCTGGATCGCGCCCCTCTGCTCTCCCTCATTTCTTGGGACGCTTCGACAG
GATTGGAACTGCATCTGAAGCGCACGCTGCACCGCGGCACTGCCCGGCGGGTTTCTGGGCGGGGAGCGATCCCCGCGTCGCCCCCCGTGAAACCGAC
AGAGCCTGGACTTTCAGGAGGTACAGCGGCGGTCTGAAGGGGATCTGGGATCTTGCAGAGGGAACTTGCATCGAAACTTGGGCAGTTCTCCGAACCG
GAGACTAAGCTTCCCCGAGCAGCGCACTTTGGAGACGTGTCCGGTCTACTCCGGACTCGCATCTCATTCCACTCGGCCATAGCCTTGGCTTCCCGGC
GACCTCAGCGTGGTCACAGGGGCCCCCCTGTGCCCAGGGAAATGTTTCAAGCTTTTCCCGGAGACTACGACTCCGGCTCCCGGTGTAGCTCATCACC
CTCCGCCGAGTCTCAGTACCTGTCTTCGGTGGACTCCTTCGGCAGTCCACCCACCGCCGCCGCCTCCCAGGTAAGTTCTAGATCGTAAAGATTCTAC
TTTAGTGGTTGGGGGGGGGTGTTTCTTTCAGGCTAAAGTGTAGATTGAGCATCCTCTCAAATAGAGACGCGCCAAAACCCAGGATCTGGGATTGCAA
TAGTTCGGTTTGCACTTTGGTTTTTTGTTGTTTGCAAACTGCAAAGAATGGAGTGATGTTTGCAAAAGGTTATTTGCGCGGAGCGCGGGAAAGGAAT
GCAGCTGGGCAAACGTTGGCGATGCCCGGTGCAAAGTATATACCCGGTGGTTAGCAGAAGCTGAGAACTTTTAGCCGAAAGCCGGCTCCCTAAGCCG
AAGCTAGGCAAGTAGGGGAAGAAAAAGAAACAAAAAATTCCAGAGAAGCTTCCAGGAGCCTCCTCCTCTTCCCTCTTCCTTCAAAACGCGGACTGCA
AGTCCGCAGTCACCCTCCACCCAGCAAGAGTTAGGGCCTCGAACCCCGGTCACGCTGCCTCCGCCTCCTGCGCGGAGACGTAACGGGGGACCCGTGC
GTAAAGGCTGACGCGCTGGAATCCTCCGTCTGACGCGGGGCACGCACAGCGCGCAGCGCCCCCTCCGCCCGCCCCGCCCCTGACGTCCCGGGCACGT
TCTATTTTGGAACGCCGAGGCCACGTTGCTAAGGGAGGGGGCAGCGTGGCTTTGTGATTGGCTGTCGCGGCGCGAGCTTTAGCCAATCAGCGTTCCC
TTCCTATTTGTAGAGCGTAGCTCCCTTCCTTGCTTTTTGTGGTTCTTCCCGTGCTGGGGGTCTCCAAGAGGGAGAGCTAGGGGATTCTTGTCGCGAT
CGGGGGACTCGTTGTCACCCCATGGGTCTGCGAGGACCTTGTGTGGACCTGGTCCTGTTGTCATAAGCTAGAGGCTTTTGGCTGAGTGTTAGCGCCT
CTAAGGGGGAACTGAAGGCCTCATCCCTTCTCCAGGCACACATATACGTGCTCCCTGAGCTCTAGACACTCAGTCCTTCCGAGGTGTTCAAACACTA
GATGAGCTAGCCTACGGAGAGGCAGCCAGGTGGTCTCTAAAAGGTCCGCCTTCCCTTAGTTCCCAGGGCTCTGATTGGCCAGGGATTCAGCCCTTCC
CTCGCCACGCCCCCTAGAGTAGTTAAGCCTCTAGGATTCCACTTGCGGGAAGGGGGGGGGGGGCGTGATGGACGCTTCTTGGGTTCGGGGACGCAGA
TCCTATGTCACCCCATCCCCTGCAAGACAGTCTGAGAGATTCTCGCTGTCACTTTTCTCTGCCTATCAGTTCACTGAAACCTGTCAGTCTCACTGGG
GAAGAGACAGACACTCGGAAGGGATGCTCTCAACTCTTAGGCCGGTCCCCCAACACCGTTGGAACTGGGATCTCCGCCCCTGCGGGAGCCCTCATGC
AGTGGGGGGTGTGTTTGTGTGTGAGTGGAGGAGAGGAAGGCTTGGGCTAAGGCCTCTCCCTCTCCCTACCTACTGTGGTGGGGGTGGGGTGTTTTGG
CTGTATGTGTGTGTGANNNNNNNNNNNNNNNNNNNNCTGACTTGAAGGGGGTAGTGTGTGTACATTTTTCTTTTTGAGACAGGATCTCACTGTATAG
GCCAGACAGGCCTGGAACTCAATCTGTAGACTAGGCTGGTCTCGAACTCACAGAGATCCACCTGTGTCTGTGGCTCCAGTCCCGGGATTAAAGGTTT
GCATCACCAGGTTCTGTCCAGCCTCCGTCTCCCACCCACCCCCCCACACCTAAGAGTCACCAACCCGGGGTGTGATTCACCACCCGCTGGAACCGTG
CAACCTTTCCCCGAGGAAGAAGGAGGAGGTAGAAGGCAGTTGAACAGAATCTCTCATTAACCACTGCGTCACGGTGTAGTGGAAGGGTGGGTGTTGT
GGCTTTTTGCCTGTGACACACACATCCACACCCGCTCACCCTGTGCTCACTCACAGGGGTCGGTGTGTGTTATGTGTGTTGGGCGTGTGTGTGTCGG
TGGCTTTGTTTGTGTGTCTACGCCTGTGTGTGTATGTCTCACCCCGTAGGAGTGCGCCGGTCTCGGGGAAATGCCCGGCTCCTTCGTGCCAACGGTC
ACCGCAATCACAACCAGCCAGGATCTTCAGTGGCTCGTGCAACCCACCCTCATCTCTTCCATGGCCCAGTCCCAGGGGCAGCCACTGGCCTCCCAGC
CTCCAGCTGTTGACCCTTATGACATGCCAGGAACCAGCTACTCAACCCCAGGCCTGAGTGCCTACAGCACTGGCGGGGCAAGCGGAAGTGGTGGGCC
TTCAACCAGCACAACCACCAGTGGACCTGTGTCTGCCCGTCCAGCCAGAGCCAGGCCTAGAAGACCCCGAGAAGAGACAGTAAGTATGAGGCCTCAG
GAGTTGGGATGGAGGAGCCTAGCTAGGGATGTGGGCTCAGTTTGTACAGTGCTTGCTGCCATGCATGAAGATCCCTAGCACAGCATAAGCCAGGAGT
GGTTATGCAGACCTGTAACCCCAGCTCTCAGAAGGTGGAGGCAGGAGGAGCAGGAGTTCGAGGCCAGCCTGTGCTACTTATGGAGTCCAGCCTGCAC
TGCAAGAGATCATTATTTTCAAAAGTTGGCCTTGGGGGGAGGTGGGTGAGGGAAGTAAGAGAAAGTGACAGTAATTTTATCACTTAATAGTTGGAGG
TTCCTCTGAGGCCTCAAGTCTGAAGGAACTTTACCATTCTGGCCAGTGAGGAGTAGGGGTTATTATTTGGGGTTCAGGAGGAAGGAGTTTTCTTAGG
GCTGATAGAGGTACCCCCAGATCTCATGGTCCTTATCTCTGACTCAGCTTACCCCAGAAGAAGAAGAAAAGCGAAGGGTTCGCAGAGAGCGGAACAA
GCTGGCTGCAGCTAAGTGCAGGAACCGTCGGAGGGAGCTGACAGATCGACTTCAGGCGGTAAGGAGGAGTCTGGGGGTGTCTTGAGGCCGTGCTGGG
AGCACTCTGCCTTGTTCTTCCCCCGTTTCTCACTGTGCCTGTGTCCTAAACGAGGAAACCCCCTCTTAGGGAACAGGGGTCAGTATAGGCTGATGGA
GTGGCTCCATATGCATGCTCAGACCCATGCCCACTTACTTTCGACTGTTCCCCACTTTCCCTGAATATGTCCCCACATGTCACCCTCCTGGCTTTCT
CTCAGCCTAAGGAGACAAGCTGGAGGAGGTAATTCTCTCACCTTCTTTCCTTCACTAAATAATAATCCATTTTGCCTTCCTGCCTCCATTTTTTTTT
CCTGAGCTGGGGATCTACCTGTGTAGTTCAGCCCTCCTCCCCCAACTTGATAGCCTCAAGTTTCAGCCCTTGGCTGAGATGCCATCATCCTGACTGG
CTCTGGCTGGAAACTATTTTGTGCTAAGTCAATTCCTTACCTGCTACTCCAGCTATCTACAGTTCTGCCGAACTTGAGCTGGTGGCGCCCACCAAGC
CCACTTCTTTCTCTCTTCTACCTCAGTGCAACCCCCACACACACACACACACCTCATGCCTGCCCCTTGAAACCAGGGTGTGTCTCTGATTTCCCGT
CGGGAGGCTGAAGGAGATGGGTAACAGAACCTCATTAAAAACAACACATAAGCATTACCTACTGACTCAACAAACTGTAGTGTTTTTCTTTTTTCCT
CTCAAAAAATTATTTCGTTTGTTTATTTATTATTTGCTTATGTTTGAGTGAGTGCTGGTGCACCACAGCACACATACGAGGTCAGAGGGAAATTTTC
ATAGTTTGTTCTCTCCTTCCGTGTTGTGGGTGCTTGCTGGCAATCTCCTTCACTCAGTGAGCTACAATGCCCCCTTCTGCCCTTTAAGGCAGAGTAC
TCCTTAGTACAGGGGGACCCTTTCCTCGGCCTCTCAAAGTTGAGATTACAAATGTTCACCATCACACCAGGCTTGGAGTTCTTGCCTATCAGTGACG
TCCACTCCTGCCTAGCTTCTTCCCAACCATCTGTTAGTCTGATGGGGAAACCGAGGCACGAGTAGCATGGTCTACCAGGATTTCCTCTTAGGGGACG
GTCCCCTCAGTTGGGAGGGAGCTGTCCAGCCCCCTGGATCAGCAGCAAGAATGTATGAGTGTGGGGTTGGGCGGGTGAAGCTACTCTGTGTGGTCGC
TGACCAGCAATTCTCCTTTCTCTGTCTCCTATGACCTGGCCCTGCTGGGATCCATTAGGAAACTGATCAGCTTGAAGAGGAAAAGGCAGAGCTGGAG
TCGGAGATCGCCGAGCTGCAAAAAGAGAAGGAACGCCTGGAGTTTGTCCTGGTGGCCCACAAACCGGGCTGCAAGATCCCCTACGAAGAGGGGCCGG
GGCCAGGCCCGCTGGCCGAGGTGAGAGATTTGCCAGGGTCAACATCCGCTAAGGAAGACGGCTTCGGCTGGCTGCTGCCGCCCCCTCCACCACCGCC
CCTGCCCTTCCAGAGCAGCCGAGACGCACCCCCCAACCTGACGGCTTCTCTCTTTACACACAGTGAAGTTCAAGTCCTCGGCGACCCCTTCCCCGTT
GTTAGCCCTTCGTACACTTCCTCGTTTGTCCTCACCTGCCCCGAGGTCTCCGCGTTCGCCGGCGCCCAACGCACCAGCGGCAGCGAGCAGCCGTCCG
ACCCGCTGAACTCGCCCTCCCTTCTTGCTCTGTAAACTCTTTAGACAAACAAAACAAACAAACCCGCAAGGAACAAGGAGGAGGAAGATGAGGAGGA
GAGGGGAGGAAGCAGTCCGGGGGTGTGTGTGTGGACCCTTTGACTCTTCTGTCTGACCACCTGCCGCCTCTGCCATCGGACATGACGGAAGGACCTC
CTTTGTGTTTTGTGCTCTGTCTCTGGTTTTCTGTGCCCCGGCGAGACCGGAGAGCTGGTGACTTTGGGGACAGGGGGTGGGGCGGGGATGGACACCC
CTCCTGCATATCTTTGTCCTGTTACTTCAACCCAACTTCTGGGGATAGATGGCTGGCTGGGTGGGTAGGGTGGGGTGCAACGCCCACCTTTGGCGTC
TTGCGTGAGGCTGGAGGGGAAAGGGTGCTGAGTGTGGGGTGCAGGGTGGGTTGAGGTCGAGCTGGCATGCACCTCCAGAGAGACCCAACGAGGAAAT
GACAGCACCGTCCTGTCCTTCTTTTCCCCCACCCACCCATCCACCCTCAAGGGTGCAGGGTGACCAAGATAGCTCTGTTTTGCTCCCTCGGGCCTTA
GCTGATTAACTTAACATTTCCAAGAGGTTACAACCTCCTCCGGGACGAATTGAGCCCCCGACTGAGGGAAGTCGATGCCCCCTTTGGGAGTCTGCTA
ACCCCACTTCCCGCTGATTCCAAAATGTGAACCCCTATCTGATTGCTCAGTCTTGCCCTCCTGGGAAAACTGGCTCAGGTTGGATTTTTTTCCTCAT
CTGCTACAGAGCCCCCTCCCAACTCAGGCCCGCTCCCACCCCTGTGCAGTATTATGCTACGTCCCTCTCACCCTCACCCCCACCCCAGGCGCCCTTG
GCCGTCCTCGTTGGGCCTTACTGGTTTTGGGCAGCAGGGGGCGCTGCGACGCCCATCTTGCTGGAGCGCTTTATACTGTGAATGAGTGGTCGGATTG
CTGGGTGCGCCGGATGGGATTGACCCCCAGCCCTCCAAAACTTTCCCTGGGCCTCCCCTTCTTCCACTTGCTTCCTCCCTCCCCTTGACAGGGAGTT
AGACTCGAAAGGATGACCACGACGCATCCCGGTGGCCTTCTTGCTCAGGCCCCAGACTTTTTCTCTTTAAGTCCTTCGCCTTCCCCAGCCTAGGACG
CCAACTTCTCCCCACCCTGGGAGCCCCGCATCCTCTCACAGAGGTCGAGGCAATTTTCAGAGAAGTTTTCAGGGCTGAGGCTTTGGCTCCCCTATCC
TCGATATTTGAATCCCCAAATATTTTTGGACTAGCATACTTAAGAGGGGGCTGAGTTCCCACTATCCCACTCCATCCAATTCCTTCAGTCCCAAAGA
CGAGTTCTGTCCCTTCCCTCCAGCTTTCACCTCGTGAGAATCCCACGAGTCAGATTTCTATTTTTTAATATTGGGGAGATGGGCCCTACCGCCCGTC
CCCAAAAAGCACTTATATCTATTATGTATAAATAAATATATTATATATGAGTGTGCGTGTGTGTGCGTGTGCGTGCGTGCGTGCGTGCGAGCTTCCT
TGTTTTCAAGTGTGCTGTGGAGTTCAAAATCGCTTCTGGGGATTTGAGTCAGACTTTCTGGCTGTCCCTTTTTGTCACTTTTTTGTTGTTGTCTCGG
CTCCTCTGGCTGTTGGAGACAGTCCCGGCCTCTCCCTTTATCCTTTCTCAAGTCTGTCTCGCTCAGACCACTTCCAACATGTCTCCACTCTCAATGA
CTCTGATCTCCGGTCTGTCTGTTAATTCTGGATTTGTCGGGGACATGCAATTTTACTTCTGTAAGTAAGTGTGACTGGGTGGTAGATTTTTTACAAT
CTATATCGTTGAGAATTCTGGGTGGAAATGTCTGATCAGGAGAAGGGCCTGCCACTGCCGACCACAATTCATTGACTCCATAGCCCTCACCCAGGCT
GTATTTGTGATTTTTTTCATTTTGTTTTTTTGTATTTrGCACCTGACCCCGGGGGTGCTGGGGCAGTCTAGCACTGGGCAGCTCCCCTCCCCCCCTT
GGTTCTGCACTGTCGCCAATAAAAAGCTTTTAAAAAACTGTATTCTTCAGGTCAAAGTGTCTGTTTTCCCTGGACATCTACTACATGGCTTCCTTTC
AGAAAAACGGAGTTTGGATTGCTAGGGAGGTCTTGCTGGCACTTAGTGGGACGCCTAATGAATCAGAACCTACAACGGGACTAAAAGGAAGTGGAGA
CTTGCTAGGTTTTCCCATGTTCCCAGGCTGGGCCACCTACTTGAAAAAATAAGGGGCGGAAAAGTGTAAGGTACAAATTTGGTGAAGGGTCTGGAAG
ACTTCATGATCGGAAAAGAATTTATTCACCTTGGGTGTGCAATGACTTCAGCAACAGCTAAGGGCAAGGTGTAAAAGCTGGGCACACTTGTAAATCC
TAGCATTTGAGAGGTGGAGGCAAGGGGATCACTGGTGGAGTTCAGTGTCATGTGGATCGTAGATACCAAGCGCAAAGATCTGCTATGGGGAGAGGGC
TTGGTACACCAGGGGAGCCAGAAGTTCGTGGTGAGGGTAGTGGAGGGCAGGTGGAGAGTGAGAGTTAGCCTCAGGGAGATTCTACAGGCAATGATGC
AGAGTTCAGACGCTCCTTTGAAAGCACTAGAGAGCCGCAGCAGGTTTTGAGCAGAGAAGGTTAGCGTTAGGTGGTCTCTTCTAGCCCATCCCAGGCT
GAGGAGGACGCTGAGGGTTTCAAGAAGGATCGAGAATGGAAAGCAGAGGAGAAAAAGGATCCAAGAGGCATGGAGGAGGCAGAACACATTTCTCTTC
TTTAATAGCAAGCCTGGAAAGGATAACTTGCTGCAGGAGGAGATGCTCACCAGTCGGGTGGTCTAGGGGGTTCTTGGAAAAGAGAAGGCATTTGCTC
AAGCCTCGGTTCCCCCATTCTCGCTCTTCTGTCAGCTTGTCTTCCATTAAGTGTGTGTCTCAAGGCCACCCTGCTCAGGACTCCCTGTGAGACGACC
TTCTATGCTCGAGTTCATTAAAAACACAATTGCCTGGTGCCCTGCTCTCTCCACTGGCTCAGTTACCTCAAAAGACCAGGGCTAAAGGTGTGATCAC
AACTCTATCCCCATTACTGCTCCAACGCAGAGACAGGACTGAGCCGGAGTGAACAAATGAACAAAAATGACTAATAATGCATGCGTGATTAAATACA
TAAAAGAGCAGATGACTGGATGAGCAAATCGTTTAAGGAGAGACAGCAAGATCCTAGAATTTTGGAGACTAATTTAAATCCATCTTTGAGATGTATT
TGGTCGGAAATTCCTGGGAGGAAAAAAAGTGTAAGTATGAAGAGAGAATAAATGAGAATAGGGGTGGCTTCAGAGAGGTTAACTGCGCGCTGGTCGC
TTTTGTACAAGAATGTGAATTGCAGGGAGCAAAATGGGATAGATACTCCCGCCCGAAAGGTGGAATTGAACCACTCTGTCGCTAAACAGCTACAGGT
TTGAAGCCTGCACCCCAGACCACTGAGGATCATCCGGGCGAAAGGAGCTATTTTCGGTTAGTTATATAAAGGCCAGATACTACTACTTTTTTCACTT
ATGGTCATTATTTGTGGTATACAGTAGATAATTAATTTCAATGGTTTCGAACATTTTTTTTCACTTTTTCTTGTGAACATGTGTTTCCTCAGTAAAG
TGTTCCGTGAATGACTCTACTAACTAAAAAGTAAGTAGCTTCATTTGCATAGCGCCTTGCATTCTGGGAAGCAGCGCCTAAAGTGCCTGTCTCCCTA
ACTAAAAGCAGAATTTTTTGCAAAGTGAAAAGTCAGTTTTATTTTTGTTTGTTTGTTTGTTTGTTTGTTTTTAATGGAAAAACTTCTCACGCGGCCC
ATTCGTAGCAGAATTCGAGATTTTCTGCAAGCGAGACCGTAGGGTCTGACGGCACGCGGCCGCAGAGCGACACCTGCCGTTGCTTTATAGAACTGCA
AGTATGTAGGGAATCTACTGAGTCCCTAAGGTGATGGAGTTGACAACCAACTCCCCTTGCGTTTAGACGCTAAAAACCATCCCTTTTTATATCTATG
TGATTAGCCCAGGGAAACTAAGGCTCAGACATGGATAATACCACAGCCGAGTTCCTGTAGCCCAACTCCCTAGGGGAAATGAAACCTACAGTTGTGG
TTTTAATATGCTTGGCCCAGGGGCAGTGGCCCTATTGGCAGGAGTGGCCTTATTAGAGGAGGTGTACCTTGTTAGAGGAAGTGTGTCACTTGGAGGC
GAGGTTTTGAGGTACGTATGCTCAAGTCTGGCCAGTGTGATCCTGGCTGTCTGCAGAACGCGGTCTCCTGGCTGCCTTCGGATCAAGGTGTAGAACT
CTCAGCTCCTTCTCCAGCACCATGTCTGCCTGCTTAATGCTTTGCTTCTTTCCATGACGATAATGAACTGTGCCTCTGAAACTGTAAGTCAACCCCA
ATTACACAATTACATGTTTTCTTTTATAAGAGTTGCATATATATATATATATGTCACACTCTTTAGCTCTGGCTGGCCTGAAATTCACTATGTAGCC
CAGGATTGCCTGAACTTTGAAGCAATCTTCCTGCCTCAGCCTCCCAATGGTATTACAGGCATGAGTCACAACAAGCCATTTAAATCTTATGATGACT
TATAAGAAGACAGAAAATCAGAGTTCCTTTACCTAGTTCACAGATCCCTACAATCTAACCTCGTTCGCTCCATAAACAGCCCTACCCCACCCTCCTG
GAACTGCTTTGAGGAATGCTGCAGGCTCTCACAGGCACACTCCTCCTTGGTTAATCTCTTCAGCCTGGTTGCCTTCCCTCCCCATGTCCATGTGGCC
CAAAGCCTCTCATCCTGTTCTCAAATACCACTAGCTAGTAAGGCCTCCCCGACCTGACCCCGGTTTAAATATTAGAAAAGGGTCACTTTCTCCCCTG
CCACAGACAACCAAACCACCATATGCTTGTCACTTACTACCTGACTATGAAGGTGAATAGATGTCTTCACAACCTTTCTCTGAGCCTCAGTTTCCCC
ACCTGCATAATGCATCTGAGACACAGAATTCCCTAGAGCTGTGGTTCTCCTCATTCCTAGTGCTGGGACCCTTTAATACATTTCCTCATGTTGTGGT
GACTCCACCACCACCATAAAATTATTTTCATTGATACTTCATAACTGTAATTTTTTCTATTCTTATGAATAGTAATGTAAGCATTTGTGTTTCCCAG
TGATCTTAGATGACCCTGTGGAAGAGTCATTCCACCCCCAAAGGGGTCCCCACCACAACTTAAGAATTCCTGCCATAGAGGAATCACAAGGAACCAA
TGAATTACACTTTGGGTCGACTTTTGGGCTGGCCTCTGGGAGGCGNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNCTTGGATTACAAACATGTGCCACCAACTCTGGCTGCCCATTATGCATTTCTGTGG
CAGGCGTTGATGGCTATTAAACCCAAGCCTTGTATATACTAGGCAAGTACCCATCCACTGAACTACAGCCCTAGCATGGACCTCACAGGGCAATGGC
CTGACATCTGGGAAACACTATCACAGAGTATAATAATAAAACCGTAGCATGCTTTTCAGATTGAGCATTTCTGCTGGGTCACACTTGGAAAACCTTC
TTATTGTTATCATGAGTATGTGTGCATACATGTGGAGGTGAGAGGATGACGTCATAGAGACAGCTCTGTCTCCCATTCTTTATTGCCAATATTATTT
TATGTTTTGTCTGCGTGTATGTTTGTATACCAAAGTGCATGCAGTGCCTGTAGAGACCAAAAGGGGGCATCCCTGAGACTGGAGTTAGAAGTGGCTG
TGAACCACTCACTCTGTGGGTGCTGGGAATCAAACCCCGGTTCTCTGGCAGAGCAACTGGTGCTCTTAACCACCGAATCAACTTCTCCAGCCCTGCA
TTGCTTTAATCTTTTGGTCGATTCCCCCAATCCAACTCATACCGCCAAGTCTGAGTAGCAAGTGCCTTTATCTGGGAGACTCACTGATCTTATTTTT
TTTTCCTTTCTTTTTCTTTCTTTTTTTCTTTTTTTTTTTGAGTCAGGTATGATTATTTAGATGGCCTCGAACTCACAGAGATCCACCTCCCTCTGTT
ATGCCAGGCTTTTAAAAAACAACATTTATTTTTGATGCATATATAGGGCTGTGCAAACACAGCCTGAAGGAGTCGTTTCTGATTGGCTCACAACGTG
GTTCCAGGGATGGAACGCGGGTGACCAGCTTGAAGGTAGGTGCTTTCACCAGCCCCAAGAATTTATTTTTTAACTTTATTTTTTTAATTAAATGATT
AAATGACACATGTTCATTGTGGGTAGTGCAGGTATGCGATGGAGCACATGTAGAGGTGACATTACTGCAGGCGGTCTCTCTACCATGGGGATTCAAC
TCAGACCACTGCTTCCTTTACCAGATCATCATTCTTGCATCCCCCAATCATTTCTTTTTGTTGATGGTTTTGTTTGTTTGTTTTAGTTTTGGGGGGT
TTTTAAAGTTTTTTTCGAGACAGNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNATTAAAGGCGTGTACTACCACTGCCTGGGCAT
AACCGGCCTTTCTGCCTTATTTGTTTCGAAACGGAGTCTCATGTAGCCTGAGGTGGCCTCAAACATGCAATGTAGCTGCAGCTGGGATTACATATGT
TCACCACCACAACCTGTTTTATGCACTGCTAAGAGATTAAACCTGGGCCTTCGTGCTAGGTAAGGACTCTATTAACAGAGCTACAACCTCTGCCTGG
TTTTTAGTTTTTCAGACAGGGCTTTGCGACATAGCCCTAACTGGCCTTGAACATGCAATGATCCTCCTGCCTCGGCCTCCTGGGTCCTCAGATTGTT
GATGCTACACCCACATATCTCAGACCTGAGGGTTGGCAGCTTGGGCCATGCTCTGATTTGGGAGTGACATATTAACAGTAGTTTAATACGAATTTGA
ATCCACCTTACTTTTTTGGTGGCACTGACATTGAACTCATGGCCTTCCACAGGCTTTTACGTGAGAAATGTTTTACCTCCCTTCCTTGAGCCATGAG
CGAGCCCCTCACTGGTGGAGTCTATGCAGCTGGTCTGAAACTCATACCATCTTCCTGCCCCACTCTCCGAGTGCTGAGTGTCTACAGAGCTCCTGAC
TCTGCCTGGGACGTGATGTCTGTGGGACCTTGGCTATAGAAGGGAGGCAGTCACACCACCCATTTTTGGCTTCCATGTCGGCAATGGAGAGCTTGCC
ATACTCACCTGATGTTGTCTGTACAGAAGGGGGACGGTAAACAAAGCAACCACTCCTACAGATGCAGAGAGAGAGATGGGGGATGGGGGGTGTCAGG
TTCTCCTGGCAAAGGGTCAGCTCCGTTGAGGGCTTTTCCACTCCCGACCAAATTCCCAAAATAACGACTCTGAGACTCAATATTTCATGAACAAATG
CTTGGGCTAGCTTGGGCTTGTTCCCTGACAAGCCCCTATCTCATTTAACCCGTTTATTCTATTCTATGAATGCCACATGGCTGGTTACCTCGTCTCA
GTTTCATGAGACCCTCTCATCAGCACTAGGGCAAACCCTGATTCTATTACTCTCTTCCTGCAGGAGCTCGGGCCCCCTATTTCCTCCCTAAGCTAAT
AGCCCAACAGCTTTTGATTGACAGGTGATGCTTCCACACAGTGCACACGAAGATTCTCTTTACCCTCCATGTGNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNATTTTTGTAGAGAGG
GCTGAGGTCAAGTTTAAGATTAGGAAAGAGAATCAAGAATTATCAAGGATAAAGGGAGAGGTCAGGATTTACAGGGGAGCAGGGTTCATGGTCAGAA
TGAAAATGGATGAGAGAGGGCAATGCGTGAGCTGAGGTCAAGGGTCACAATGGACGGTCCGTCGGCGGGGGCTGGGCAGGGCCAAGCATTCTCACTG
GAAAGGGTTGGTGCCGTCGCCCCGGTGCACGGCCTGCAGCACTTTGCGGTAAACCCTGAGAGAGATGGTGGCGCAGAGACCCAACAGGGCCAGGTGC
GCAGCCACGGACACGATGCTAAAGTGCAGGAGGCAGAGGAGAGAGGCCATGAGGCCCGTGAAGACCGCTCCCGACGTCCTGGTGTCCTTCCAGTACA
ACAGGTCTGCCACTGTGGAAAAGAAGACAATGTTGGGACCCGACCTGACCAAGCTCACTCCCTTTCTGTTTGTTTTTTAGAGATAAGGTCTCACTAT
GAAGCTAAGGCTGACTTCAAACTATGAAACCCTGGCTGGTCTTGAACTCCTGATTATTCTAATTCATCCTTTCTTTTTCTTAGACTGAGTTAAGTTT
GACCGGCTTCAAACTCAAGACAAATACTCCTGCCTCTACCCTTTAGTGCTTCAAGGAGGTGCTAAGGATCAAATTTAGGGCTTCATGCATGCTAGGT
AAAACCCAACTGAGCTACTTCCCTGAC
MOUSE SEQUENCE - mRNA
ATAAATTCTTATTTTGACACTCACCAAAATAGTCACCTGGAAAACCCGCTTTTTGTGACAAAGTACAGAAGGCTTGGTCACATTTAAATCACTGAGA
ACTAGAGAGAAATACTATCGCAAACTGTAATAGACATTACATCCATAAAAGTTTCCCCAGTCCTTATTGTAATATTGCACAGTGCAATTGCTACATG
GCAAACTAGTGTAGCATAGAAGTCAAAGCAAAAACAAACCAAAGAAAGGAGCCACAAGAGTAAAACTGTTCAACAGTTAATAGTTCAAACTAAGCCA
TTGAATCTATCATTGGGATCGTTAAAATGAATCTTCCTACACCTTGCAGTGTATGATTTAACTTTTACAGAACACAAGCCAAGTTTAAAATCAGCAG
TAGAGATATTAAAATGAAAAGGTTTGCTAATAGAGTAACATTAAATACCCTGAAGGAAAAAAAACCTAAATATCAAAATAACTGATTAAAATTCACT
TGCAAATTAGCACACGAATATGCAACTTGGAAATCATGCAGTGTTTTATTTAAGAAAACATAAAACAAAACTATTAAAATAGTTTTAGAGGGGGTAA
AATCCAGGTCCTCTGCCAGGATGCTAAAATTAGACTTCAGGGGAATTTTGAAGTCTTCAATTTTGAAACCTATTAAAAAGCCCATGATTACAGTTAA
TTAAGAGCAGTGCACGCAACAGTGACACGCCTTTAGAGAGCATTACTGTGTATGAACATGTTGGCTGCTACCAGCCACAGTCAATTTAACAAGGCTG
CTCAGTCATGAACTTAATACAGAGAGAGCACGCCTAGGCAGCAAGCACAGCTTGCTGGGCCACTTTCCTCCCTGTCGTGACACAATCAATCCGTGTA
CTTGGTGTATCTGAAGCGCACGCTGCACCGCGGCACTGCCCGGCGGGTTTCTGGGCGGGGAGCGATCCCCGCGTCGCCCCCCGTGAAACCGACAGAG
CCTGGACTTTCAGGAGGTACAGCGGCGGTCTGAAGGGGATCTGGGATCTTGCAGAGGGAACTTGCATCGAAACTTGGGCAGTTCTCCGAACCGGAGA
CTAAGCTTCCCCGAGCAGCGCACTTTGGAGACGTGTCCGGTCTACTCCGGACTCGCATCTCATTCCACTCGGCCATAGCCTTGGCTTCCCGGCGACC
TCAGCGTGGTCACAGGGGCCCCCCTGTGCCCAGGGAAATGTTTCAAGCTTTTCCCGGAGACTACGACTCCGGCTCCCGGTGTAGCTCATCACCCTCC
GCCGAGTCTCAGTACCTGTCTTCGGTGGACTCCTTCGGCAGTCCACCCACCGCCGCCGCCTCCCAGGAGTGCGCCGGTCTCGGGGAAATGCCCGGCT
CCTTCGTGCCAACGGTCACCGCAATCACAACCAGCCAGGATCTTCAGTGGCTCGTGCAACCCACCCTCATCTCTTCCATGGCCCAGTCCCAGGGGCA
GCCACTGGCCTCCCAGCCTCCAGCTGTTGACCCTTATGACATGCCAGGAACCAGCTACTCAACCCCAGGCCTGAGTGCCTACAGCACTGGCGGGGCA
AGCGGAAGTGGTGGGCCTTCAACCAGCACAACCACCAGTGGACCTGTGTCTGCCCGTCCAGCCAGAGCCAGGCCTAGAAGACCCCGAGAAGAGACAC
TTACCCCAGAAGAAGAAGAAAAGCGAAGGGTTCGCAGAGAGCGGAACAAGCTGGCTGCAGCTAAGTGCAGGAACCGTCGGAGGGAGCTGACAGATCG
ACTTCAGGCGGAAACTGATCAGCTTGAAGAGGAAAAGGCAGAGCTGGAGTCGGAGATCGCCGAGCTGCAAAAAGAGAAGGAACGCCTGGAGTTTGTC
CTGGTGGCCCACAAACCGGGCTGCAAGATCCCCTACGAAGAGGGGCCGGGGCCAGGCCCGCTGGCCGAGGTGAGAGATTTGCCAGGGTCAACATCCG
CTAAGGAAGACGGCTTCGGCTGGCTGCTGCCGCCCCCTCCACCACCCCCCCTGCCCTTCCAGAGCAGCCGAGACGCACCCCCCAACCTGACGGCTTC
TCTCTTTACACACAGTGAAGTTCAAGTCCTCGGCGACCCCTTCCCCGTTGTTAGCCCTTCGTACACTTCCTCGTTTGTCCTCACCTGCCCGGAGGTC
TCCGCGTTCGCCGGCGCCCAACGCACCAGCGGCAGCGAGCAGCCGTCCGACCCGCTGAACTCGCCCTCCCTTCTTGCTCTGTAAACTCTTTAGACAA
ACAAAACAAACAAACCCGCAAGGAACAAGGAGGAGGAAGATGAGGAGGAGAGGGGAGGAAGCAGTCCGGGGGTGTGTGTGTGGACCCTTTGACTCTT
CTGTCTGACCACCTGCCGCCTCTGCCATCGGACATGACGGAAGGACCTCCTTTGTGTTTTGTGCTCCGTCTCTGGTTTTCTGTGCCCCGGCGAGACC
GGAGAGCTGGTGACTTTGGGGACAGGGGGTGGGGCGGGGATGGACACCCCTCCTGCATATCTTTGTCCTGTTACTTCAACCCAACTTCTGGGGATAG
ATGGCTGGCTGGGTGGGTAGGGTGGGGTGCAACGCCCACCTTTGGCGTCTTGCGTGAGGCTGGAGGGGAAAGGGTGCTGAGTGTGGGGTGCAGGGTG
GGTTGAGGTCGAGCTGGCATGCACCTCCAGAGAGACCCAACGAGGAAATGACAGCACCGTCCTGTCCTTCTTTTCCCCCACCCACCCATCCACCCTC
AAGGGTGCAGGGTGACCAAGATAGCTCTGTTTTGCTCCCTCGGGCCTTAGCTGATTAACTTAACATTTCCAAGAGGTTACAACCTCCTCCTGGACGA
ATTGAGCCCCCGACTGAGGGAAGTCGATGCCCCCTTTGGGAGTCTGCTAACCCCACTTCCCGCTGATTCCAAAATGTGAACCCCTATCTGACTGCTC
AGTCTTTCCCTCCTGGGAAAACTGGCTCAGGTTGGATTTTTTTCCTCGTCTGCTACAGAGCCCCCTCCCAACTCAGGCCCGCTCCCACCCCTGTGCA
GTATTATGCTATGTCCCTCTCACCCTCACCCCCACCCCAGGCGCCCTTGGCCGTCCTCGTTGGGCCTTACTGGTTTTGGGCAGCAGGGGGCGCTGCG
ACGCCCATCTTGCTGGAGCGCTTTATACTGTGAATGAGTGGTCGGATTGCTGGGTGCGCCGGATGGGATTGACCCCCAGCCCTCCAAAACTTTCCCT
GGGCCTCCCCTTCTTCCACTTGCTTCCTCCCTCCCCTTGACAGGGAGTTAGACTCGAAAGGATGACCACGACGCATCCCGGTGGCCTTCTTGCTCAG
GCCCCAGACTTTTTCTCTTTAAGTCCTTCGCCTTCCCCAGCCTAGGACGCCAACTTCTCCCCACCCTGGGAGCCCCGCATCCTCTCACAGAGGTCGA
GGCAATTTTCAGAGAAGTTTTCAGGGCTGAGGCTTTGGCTCCCCTATCCTCGATATTTGAATCCCCAAATATTTTTGGACTAGCATACTTAAGAGGG
GGCTGAGTTCCCACTATCCCACTCCATCCAATTCCTTCAGTCCCAAAGACGAGTTCTGTCCCTTCCCTCCAGCTTTCACCTCGTGAGAATCCCACGA
GTCAGATTTCTATTTTTTAATATTGGGGAGATGGGCCCTACCGCCCGTCCCCCGTGCTGCATGGAACATTCCATACCCTGTCCTGGGCCCTAGGTTC
CAAACCTAATCCCAAACCCCACCCCCAGCTATTTATCCCTTTCCTGGTTCCCAAAAAGCACTTATATCTATTATGTATAAATAAATATATTATATAT
GAGTGTGCGTGTGTGTGCGTGTGCGTGCGTGCGTGCGTGCGTGCGAGCTTCCTTGTTTTCAAGTGTGCTGTGGAGTTCAAAATCGCTTCTGGGGATT
TGAGTCAGACTTTCTGGCTGTCCCTTTTTGTCACCTTTTTGTTGTTGTCTCGGCTCCTCTGGCTGTTGGAGACAGTCCCGGCCTCTCCCTTTATCCT
TTCTCAAGTCTGTCTCGCTCAGACCACTTCCAACATGTCTCCACTCTCAATGACTCTGATCTCCGGTNTGTCTGTTAATTCTGGATTTGTCGGGGAC
ATGCAATTTTACTTCTGTAAGTAAGTGTGACTGGGTGGTAGATTTTTTACAATCTATATCGTTGAGAATTC
MOUSE SEQUENCE - CODING
ATGTTTCAAGCTTTTCCCGGAGACTACGACTCCGGCTCCCGGTGTAGCTCATCACCCTCCGCCGAGTCTCAGTACCTGTCTTCGGTGGACTCCTTCG
GCAGTCCACCCACCGCCGCCGCCTCCCAGGAGTGCGCCGGTCTCGGGGAAATGCCCGGCTCCTTCGTGCCAACGGTCACCGCAATCACAACCAGCCA
GGATCTTCAGTGGCTCGTGCAACCCACCCTCATCTCTTCCATGGCCCAGTCCCAGGGGCAGCCACTGGCCTCCCAGCCTCCAGCTGTTGACCCTTAT
GACATGCCAGGAACCAGCTACTCAACCCCAGGCCTGAGTGCCTACAGCACTGGCGGGGCAAGCGGAAGTGGTGGGCCTTCAACCAGCACAACCACCA
GTGGACCTGTGTCTGCCCGTCCAGCCAGAGCCAGGCCTAGAAGACCCCGAGAAGAGACACTTACCCCAGAAGAAGAAGAAAAGCGAAGGGTTCGCAG
AGAGCGGAACAAGCTGGCTGCAGCTAAGTGCAGGAACCGTCGGAGGGAGCTGACAGATCGACTTCAGGCGGAAACTGATCAGCTTGAAGAGGAAAAG
GCAGAGCTGGAGTCGGAGATCGCCGAGCTGCAAAAAGAGAAGGAACGCCTGGAGTTTGTCCTGGTGGCCCACAAACCGGGCTGCAAGATCCCCTACG
AAGAGGGGCCGGGGCCAGGCCCGCTGGCCGAGGTGAGAGATTTGCCAGGGTCAACATCCGCTAAGGAAGACGGCTTCGGCTGGCTGCTGCCGCCCCC
TCCACCACCCCCCCTGCCCTTCCAGAGCAGCCGAGACGCACCCCCCAACCTGACGGCTTCTCTCTTTACACACAGTGAAGTTCAAGTCCTCGGCGAC
CCCTTCCCCGTTGTTAGCCCTTCGTACACTTCCTCGTTTGTCCTCACCTGCCCGGAGGTCTCCGCGTTCGCCGGCGCCCAACGCACCAGCGGCAGCG
AGCAGCCGTCCGACCCGCTGAACTCGCCCTCCCTTCTTGCTCTGTAA
HUMAN SEQUENCE - GENOMIC
CACTAAGCACTCTCACCACCCAATGCCTGGAGTGGTTGTAGTCAGTGAGTGACACATTGCACAGTGTGTGCCCCCTGGATTGGGGGTGGGTGAGAGA
CAGCCCCCACAGTGAGTGGCACCCCTGGCCAGGGCCTGGGACAAGTCTGTATCCAAGGGTGGCTCTCTGCTTAGGTCTGTGTTTGTACCTGGGTGTG
TCTGTGTCTGCCCTACTCTGTGCATACTCATATATGTGAACCCGTGTGTGTGTGTGTAGTTGTGAGTGTGTGTGGAAGAGGCTGCATGGCAGTGGAA
GCTAGGTGTGTGATTCGTGTATCTGTGTGCCTAGAGTGCCTTGTTCTATAGATTTGGATGCCACTCCAAGCAAGTCAGTGGGTCTTTTTGTTTTTTG
TTTTTTGAGATGGAATCTCACTCTGTTGCCCAGGCTGGAGTGCAGTGGCACGATCTTGGCTCACTGCAACCTCTACCTCCTGGGTTCAAGTGATTCT
CCTGCCTCGGCCCCCCGAGTAGCTGGGATTACAGGTGCCCACCAATACACTCAGCTAATTTGTTGTATTTTTAGTAAAGATGGGGTTTCACCATGTT
GGCCAGGCTGGTCTCAAACTCCTGACCTCATGATTCTCCTGCCTCGGCCTCCCAAAGTGCTGGGATTACAGGCATGAGCCACCGCGCCCGGCCAAGT
CAGTGGGTCTTTAGGAGCTGTTTCGTACGGTGTGACTGTGAGTGAACCTTCGCACACGTGTCTGTACGGATATCTAAGAAATICTTCAAGTAGGCCG
GGCACAGTGGTTCAGGCCTGTAGCCTTAGCACTTTGGGAGGCCCAGGTGAGTGGATCGCTTGAGCTCAGGAGTTCGAGAACAGCCTGGGCAACATAG
TGAGACTTCGTATCTATAAAAAATATGAAAATTAGCCAGGCATGACAGTGGGCACCTGTAGTCCCAGCTACTACTTGGGGGGCTGAGGCAGGAGGAT
CCCTTGAGCCTGGGAGGTGGAGGCTGCAGTGAGCTGAGATCGAGCCACTGCACTCCAGCCTGGGCGACAGAGGGAGACCCTGTCTCAAAAAAAAAGA
AAAGAAAAGAAAAAAAGAAGACATTCTTCAAGTCCACAACTCTGAGGGTATCACTGTGAGAGCAGGGGTCCTAGCTCTACCCATTTGTGTGTGTGTG
GAGATGTGTAAGTCTATGTAGACAAAAGTGTGTGTCATTTACTGTGTTTGGGGTGTGAACACCTATGTGATGTGTTTGCACAACTGCACGTGTTTTG
TTCTGTGTGTGTGATCGTGTGTTCAAATAAGTCATCTTGTCGCCGGGCGTGGTCGCTCATGCCTATAATCCCAGCACTTTGGGAGGCCTAGGCAGGA
GGACCATTGAGCCCAGGAGGTCCAGACTGCAGTGAGCCGAGATTGCGCCACTGCACTCCAGCCTGGGCGACAGCAAGACCTTGTCTCAAAACAAAAG
CAAAAACAAAAATAAACAAATAGTCATCAGGTGCCTGCACAGACAAAGGTGAAAGGTGTCTGCCTGTTGAGATCTGTGGATAGGGTGTATATATGGA
CATCTCAGCCTGTCTACGTGTGTATCTGTCTCTGTCCTCACGGCAAAAGAGAGGTTGGCCGGGTGTGTGGTGGCTCACGCCTGTAATCCCAGCACTT
TGGGAGGCTGAGGCGGGCGGATCACCTGAGGTGAGGAGTTCAAAACCAGCCTGGCCAACATGGCGAAACTCCATCTCTTCTAAAAATACAAAAAAAT
TAACCGGGCGTGGTGGCACACGCTTGTAATCCCAGCTACTCAGGGAGGCTGAGGCATTAGAATTGCTTGAACCCAGGAGGTGGAGGTTGCAGTGAGC
CAAGATTGCGCCACTGGACTCCAGCCTGGGCAACAGAACAAGACTCCGTCTCAAAAAAAAAAAAAAAAAAAAAAAGAGGCTCAGATATGTTTCTGTC
TGAGAGTCTGTCAGAGTTTAGGAATTAGTAAATGAATGAATGGTGAAGATCCATTTACTCAACAAACATTTATTTATTTATTTATTTATTGAGACAG
AGTCTCGCTCTGTCACCCAGGATGGAGTGCAGTGGCGCAATCTCGGCTCACTGAAGCCTCTGCCTCCTGGGTTTAAGAGAGTCTTGTGCCTCAGCCA
CCAAGTAGCTGGGATTACAGGTGTGTGCCACTGCGCCCAGCTAATTTTTGTATATTTAGTAGAGATGGGGTTTCACCATGTTGCCCAGGCTGGTCTC
GAACTCCTGGCCTGAAGTGATCTGCTCACCTCAGTCTCCCAATATGCTGGGATTACAGGCATGAGCCACCAGTCCTGCCTCATTTATTCTTATATTA
TATTATATTGTTTTTATTTTAAATTTTTTTGTAGTGACAGGGTCTTACTATGTTGACTGGGCTGGCCTCAAACTGGCCTCAAGTGATTCTCCTGCCT
CAGCCTCCCAAAGTGCTGGGATTACAGGCATGAGCCACTGTGCCTGGCTTGAACAAATATTTAATAACCACCTATTGGGTACCACGTGCTATGCTGG
GGACAAGGCAGTGACCGGGATGGTTTTGGCCGAGCTTTCACCCAGCTCACAACCCAATGAGGGAGACAAACCTATCCCCAGACAATGATGAGCCCAG
ATTGGCAGAGTTGGAGGGAGGGACCCCAGGAGAGGGGGCCTTGACTCAGCCTGGAGATCAGGGAGGGCTTCCTGGAGGAGAGGTTATGGGAGTTAAG
ACTTGGAGGAAAAGACTATGAGCCACAGGAAGCGAAGGGAAGAGTGACCCAGGCAGAGGGAAAAGCACCTGCAAAGGCCTGAAGTCCAGGGAGAGAG
GCCAGGCATCTGGAACACAACGGGGAGAGGAGAGACGAGACTAGGGCACCCTCGGAGCAGGTCGTTCAGGGCCTCAGGAGCCATGGGGAGGAGTATA
ATGCAGGATGCAGGGGTACTGGGAGGGTCAAGCTAACAGTGTCCAATCTGGGTTACTCTGCTTCCCTGAAACTCCAGGGACATCTCCTCCAGGAAGC
CCTAGGGATTGCTCAGTGTGGAGCGGCTCAGCCACCCAGTCACACTCACTATTCAATGCTGAGCACTGAGGAGGCAGCCGTTACTAAATCAGCCTGG
ATTTGCCCTCCCAGAGGTCACAGTCACAACAGTCAAACACACACCTGCACGTGCACACTCATGAACGTGCCCCTCCCCTCCTCTGGAGCCTCAGAAA
CCCACAGACCTTGTTCCTCCCAGGGGGTGTCCCTGGGGCCAAAGCTTCTGCACTACCAGGGGGCTTGTTGGAAATGCAAAAGATGGCTGGGCACAGT
GGCTCACACCTGTAATCCCAGCAGTTTGGGAAGCTGAGGCAGGCAGATCATTTGAGGTCAGGAGTTCATTTGAGGTTGGGAGTTCAAGACCAGCTTG
GCCAACATAGCGAAACTCTGTCTCTACTAAAAATACAAAAAAATTAGCCGGGCGTGGTAGCACACGCCTGTAATCCCAGCTACTTGGGAGGCTGAGG
CAGGAGAATCTTTTGAACCCGAGAGGTGAAGGCTGCAGTGAGCCAAGATGGCACCACTGCACTCCAGCCTGGGTGACAGAGTGAGTCTTAAAAAAAT
AATAATAAAATAGGCTGGGCGCGGTGGCTCACGCCTGTAATCCCACCACTTTGGGAGGCTGAGGCAGGCGGATCACCTGAGGTCAGGAGTTCGAGAC
CAGCCTGACCAAAATAGTGAAACCCCGTCTCTACTAAAAATACAAAAAAATTTAGCTGCGCGTAGTGGCTAACGCCTGTAATCCGAGCTACAGGCTG
AGGCAGGAAAATTGCTTGAACCCAGGGGTGCAGAGGTTGCAGTGAGCCAAGATGGCACCATTGCACTCCAGCCTGGGCAACAACAGTGAAACTCCGT
CTCAAAAAACAAAATACAAAAATTAGCCAGGCATGGTGGCACACACCTGTAATCCCAGCTACTCGGGAGGCTGAGGCAGGAGAATCACTTGAACCCG
GGAGGCAGAGGTTGCAGTGAGCTGAGATCGTGCCACTGCACTCAAGCCTGTGTAACAAGAGTGAAATTCCGTCTCAAAATAAAATAAAATAATAAAA
ATAAAAAGATGACCACAGCAGATCTGCTGGCTCTCCAGGCAATTCGTGATCACCACGAATTGAGAAGCGCTGCTCTCAGGCTTAACCCCCCTGTACC
TCAGTTTCCTTTTCTGTACAAGGAGATCAAAACAGAATCCATGTAGAGGACTGACGTGGAGTGAGGCTTAACTGAGATAATGAGTGTGAAAGTGCTA
AGCACAGCTTCCTAAGTGCTTGTTTTCAGCAGCTCCTCTTCCTCTTTTCTATTTTTTTATTTTTATTTTTTTTGGGGGGGGAACAGGGTCTCGCTCT
GTTGCCCAGGCTGGAGTATAGTGGCAGAGTCATAGCTCACTGCAACCTCAATCTCTCAGGGCTCAAGTGATGCCCGTGCCTCAGCCTCCTGAGTGGC
TGGGACTACAGGTGTGTGCCACCATGCCCAGCTAATTTTTAAATTTCTTGTAGAGGTTGGGGGTGGGTCTCACTATGTTGCCCAGGCTGGTCTTGTA
CTCCTGGCCTCTGGCGATCCTCTTACCTAGGCCTCCCAAAGTGCTGGGATTACAGGCGTGAGCCACCACACCCAGCCCTCCTCCTCCTCTGTATTAT
TAAATTTCAGAGCTGGAAAGAAATTTCGAAGACCGCCATCACTCAACCTTCTTGCTCTACAGATGGGGGAACTGAGGAACAGAGGAGCAGAGGTTTG
GCCAGAGGAAGGTGGGAAGGACGTGGTGTGGCTGGCCTTCACAGACTCTGCCGGGGGCTGTTTACAGGATACTCTGAGGAAAGCCAGGATGGGCACT
GATACCCACCTCCACCCTGCGGGAGGCAGAAGGCTAAGGCTCCCCCAGCCGTCTCCCCCAAGCAGAGCTGAGCCCTTGGGAGCCAGGGCAGAGATGC
AACCCTCTCCGCCATGCTCAGGTCCTCCTCCTCCTGGAAGCTCTCCTTCCAAGAGGAGCCTCTTATTTTATTTTTTATTATTATTATTTTTGAGACA
AGGTCTCACTTTGTCACCCAGGCTGGAAGTGCCGTGGCATGATCATGGCTCACTGAAGCCTTGCCCTCCCCAAGCTCAAGTGATCCTCTCACCTCAG
CCTCCCAAATAGCTGAGACTACAGGCACACGCCACCACCCCTGGCTAGTTTTTGTATTTTTAGTAGAGATGGGGTCTTGCCATTGTCCAGGCTGGTC
TTGAACTCCCGGGCTCAAGTGATCTGCCCACCTTGGCCTCCCAAAGTGCTGGGATTACAGGCATGAGACACTGAGCCTGGCAAGGAATGAGCCTTAT
GGAGAAGAACTTGATTTACCCACTCCATGCTCTGCAGGCAGGTGTGTCATGTCTGTGAGTGCAGGTGTGTGTGTGTGTGTGTATGTGTGTGTGTGTG
TGTCTCCAGGGCCAATTTCTCCCTTAGGCCCAGGGGCACAGTGCCTAGGGCCCATGATAAATTTAACCGACCCACGGATATGGGTCAGGAGGGTATA
CATGTATAAAGTTCAGGAAGACTTTGTTTTAGTAACAAAACGAGCATGTACAATCTGTCAGGAGCCTTATGATATCTTCCCACCAGTCTCATGAGTG
GGCACTACATCTTGTTCTCCTTTGTCGGGTAGGGAAACTGAGGCCCCGGATGTCCTAGCTCAAGTTCACCAAGCTACTTGGTGATAGAAGCGGAACA
TAAGCCTAGGCCACCTAGCTTCTGGGTCTGCGATCGTGTGAGGATAAATGCCCAGTGTGTTCGTGAACATGAGCATCCCTGTGTGAATAAATTGACA
TACATAAACCCATTTAATAAATTTCAAAGCCAGGCGCCGTGGCTTACTCCTGCAATCTCAGCGCTCTGGGAGGCTGAGGTGGGAGGATCGCTTGAAG
CCAGGAGTTTGAGATCAGCCTGCAACAAAGTGAGACCCTGACTCTAAAAACATTTTTTTGAATAAAAAAATTAGGCTGGACACAGTGGCTCACGCCT
GTAATCCCAGCACTTTGGGAGGCCAAGGTGGGTGGATCACTTGAGGTCAGGAGTTTGAGACCAGCCTGGTCAACAGGGTGAAACCCTTTCTCTACTA
AAAATAGAAAAATTAGCCAGGCGTGGTGGCACATGCCTGTAATCCCACCTACTGGGGAGGCTGAGGCCGGAAAATTGCTTGAATCTGGGAGGTAGAG
GTTGCAGTGAGCCAAGATCATGCCATTGCACTCCAGCCTGGGCGAAGAAACGAGACTCTGTCTCAAAAAAAAAAAAAAAAAATTAGTCAAGCATGGT
GGTACGCACCTGTAGTTGTTAGTTACTTGGGAGGCTGAAGCAGGAGGATTACTTGAGGCCAGGAGTTCGAGGTTACAGTGAACTATGATTGCATCAC
TGCACTCTAGCCTGGATGACAGAGCAAGATCCTATCTCAAAATAATACTAATAGTTCAAGGAACAGACTTTGAAGCCTGACACCCTGCATGGTGTTA
TTCCAGCTTTGCTACTTACTTGCTGTGTGACTCTGGGTGAATAACTTAACCTCTCTGGGCTTCTGTTTCCCTTCCTGTAAAATGATCATTTGTACCT
CACAGGAGTGTTGTGAGAATTAAATAAGTTAATATAAGCTCTTGGGAAGAATTAGCTCTTGTTATGGTTGTGTGAAGAGCTTTACACGAGTCCCTCT
GCAGGATGCATGGTTGTTGGTCTGTGTGTGTGTATATATATATATGTGTGTGTGTATGTGTGTATCTATGTGTATATGTGTATATGTGTGTGTTGCT
CTCTTGATCTCTGAGTGTTGTGTGTGTGTCAATGTGTGAGATCATATATGCACCTTTAGTAAAGATCTAGGGGTCTCTGTGTGTTTTGGGGGTCTGT
ACCTGTCCTTGGCTGTACACCTGAACCCTACGTCTTCTTGTGTGTGTAGGGATGTGTGTCATGTGTGTTGATAACCATATGACAGTGTGTGTTTCCA
CATGCCAGTGTGTGTCTGTGAAGCTGTTTCTATGTGGCTGTGGGTGATTGCCCACGCGTGACCTGGTGAGCGGCTGTGTGGAAGTCTGTGTTGCCTG
TGTCGGGTGTGGAGTCTCTGGCCAGCCAGGGTAGGTCTTTGCATGTTTCCTTTATTCCATGGAGGAGGAGATGAGGGGCTTGGATGAGACAGAAGAA
TGAGGGGTCACTCCCTGTCTGATGCTGGGGCCGAGTCACTGCCTACTAGTGGCTGCTGTGTCATCTCCCAGTCTCTGTCCCTCCCTCCTGGCTGGTG
GCAGACAGAGGGGGCGTGGAGGAACAGGGCAGGGGGTGGGCTTGAGGCTGGACTTTTGTCTGGAAACTTGAACCTCCACCTATGCCCCCCACGTCCC
TCTAGTCTTCCACATCTCCCTACACCCTCCCTACTATTGGGGCAGGGAATCAGGACTCCCTGAAAATTAAGAACCATCGCCTTGGCTCTGCCACAGG
CTTGCTGTGTAGCCCGGAGACAGGCCCTGCCCCTCTCTCAACCTGTGAAATTCAATGTTTTAAAAGTGCTTCTTCCTTTGTGCCCAGGGCTGGGCCA
GGCTGTGCACTGGGAGGGAAGTGCTGGGTTCTCTGAGGTTTGTGAAATAGCTGGTAGCACCCTTCCCAGTGGGTGCTTAAGAGTTGGGGGCTGTAGC
ATCTGAAATACCCAAGTCAGTGCAGGCATAGTGGCTCATGCCTGTAATCCCAGCACTTTGGGAGACCGAGGCAGGCGGATCACCTGAGGTTGGGAGT
TCGAAACCAGCCTGGCCAACATGGTGAAACCCCCATCTCTATTAAAAATACAAAAGTTAGGCCGGGCACAGTGGCTTACGCCTGTAATCCCAGCATT
TAAGAAGGCCAAGGCGGGAGGATCACCTGAGTTTGGGAGTTTGAGAGCAGCCTGACCAATATGGAGAAACTCTGTCTCTACTAAAAATACAAAAAAA
CTAGCCAGGCATGGTGGCACATGCCTGTAATCCCAGCTACTCGGGAGGCTGAGGCAGGAGAAGCACTTCAACCCGGGAGGCGGAGGTTGCGGTGAGC
CGAGATCGTGCCATTGCACTCCAGCCTGGGCAACAAGAGCAAAACTCTGTCTCAAAAAACAAAACAGAACAAAACAAAAATTAGCCAGGTGTGGTGG
CGCACACCTGTAATCCCAGCTACTCGGGAGGCTGAGGCAGGAGAATCACTTGAACCCAGGAGATGTAGGCTGCAGTGAGCCGAGATTGTGGCACTGC
ACTCCAGCCTGGGCAACAGAGTGAGACTCTGTCAAAAAAAAAAAAAAAAAACGTCAGATTCATGAACCCCATTTGCTAAGAAGATTTGCTCAGGTAG
TCACCTGTACCCCTGTGAGCCAAGATTGCAACAGTACATGCTAAGAGCAGTAAGAAGGAGTTTGTAAAGCTTAGAAAGACCTGACAGGCCAGGCGCT
GTGGCTCACGCCTGTAATCTCGACACTTCGGGAGGCCGAGGCAGGCGGATCACCAGGTCAGGAGTTCAAGACCATCCTGACCAATATGGTGAAACCC
CGTCTGTACTAAACATATAAAAATTAGCTTGACGTGGTGGCCCGCGCCTGTAGTCCAGCAACTCGAGAGGCTAAGGTGAGGCGGAGGTTGCAGTGAG
CCGAGATCGCCACTGCACTCCAGCCTGGCGGCAGAGCAAGACTCCGTCTCAAAAAAAAAAAAAAAAAAAAAAAAAAAAGACCTGACAAGTGAAAGCT
ACTAATATTGCCATTATCATTTAAAAAAACCCCAGACACAGGTTTTTCAGGGAGTTTCATCCAACCAGGCAGGTCTCAGAAATCAGAAAAGAAATGG
AAAGGGTAGGAAATCTGGAGTTTGACAATTCTGAGTTTGAATTTCTTGTGATGGGATCTTGGGCAAGTCATTTAACCTCCCTGAGTATCATTTTTTT
CTTTTATAAAATGAAGATTTTTCTCTCTTAACCTTCCAGAGCTGTTTTAAGGATTACAAATCTTCTACTGAAAGGCGTAGCACAGGAGCTGTGCTTG
GCAAGTGCCAAATACAAGGCATTAATTATTATTATTAGAATTAATAATAATATCCCCTCCCTCTTACACAT1CTTTGTCTCCGGGTGGATTAAAAGG
TGGAAGGAGAGGCTACCAACACCATCAGAAGAGAGGCTTCTCTCTAAGTTTCATTTCCCATCTCCTCCAAATCCGCATCCCTCCCAAACGCCGGACC
TGTAAGGCCAGCAGGGTCCAAGACACACATCCTTTGCCCAGCGGGGAAGATTAAAGCTAAAGCTCAGAGAGGGAAAACATTTCCTAAGCTCGCACAG
CGAATCAGGACAGAAACCAGGACGAGCCTCGGAATCCCTCCATTACCTCCACTTTCACCTGAGCATCACAGCCCGCTCGGGACTCAGTTTCCCCACC
TACGTGACCACACCACACTAATCAGGGTCTCCTTTTGGAGATCTGCTCTTCTTCTCGAATGGGGGCGCTGCACCATCGGTAGAACAGGGTAGGTGGG
GGCGCCAGAGGTGAAGGGGACCTGCAGGCTGGGGTCTTCCCCGCCCGGGTCAGCGGGGTCCCTGCGGGGCTAGTCTAAGCGCCTATTATTACCAGCC
CCCGGGGCGGCGTTGCACTGCGCAGGCGCGGGCGGGGCGCGGGCGCGCGCGCGAGCGAGCGAGGGATTCCCTCTGACGTCATTGCTAGGATACCAAA
CAAACACTCCGCCGCGCCGGCCGAGCTCCTTATATGGCTAATTGCGTCACAGGAACTCCGGGAAGGCGGGGCCGGGATCCCCTCCCGCCGAGTGGCC
CGGAACGCAACCCCCGAGACCCCCAGGGCCCCGAGGGTCATGCAAGTGACCAGATCGAGTCTAGAACAGACCTCTTGCTGGACAGTGCGGGACTCGA
TTTGGCGGGGCCGCAGATTTGGGGAAGTTTGTCCAGCAAGGGGCGGGTGACGTAAGCAGGGGGGCGGGTCCCGGGCATATAAATACAGGCTGGCGGG
TCTGTGCTTCATTCATAAGACTCAGAGCTACGGCCACGGCAGGGACACGCGGAACCAAGACTTGGAAACTTGATTGTTGTGGTTCTTCTTGGGGGTT
ATGAAATTTCATTAATCTTTTTTTTTCCGGGGAGAAAGTTTTTGGAAAGATTCTTCCAGATATTTCTTCATTTTCTTTTGGAGGACCGACTTACTTT
TTTTGGTCTTCTTTATTACTCCCCTCCCCCCGTGGGACCCGCCGGACGCGTGGAGGAGACCGTAGCTGAAGCTGATTCTGTACAGCGGGACAGCGCT
TTCTGCCCCTGGGGGAGCAACCCCTCCCTCGCCCCTGGGTCCTACGGAGCCTGCACTTTCAAGAGGTACAGCGGCATCCTGTGGGGGCCTGGGCACC
GCAGGAAGACTGCACAGAAACTTTGCCATTGTTGGAACGGGACGTTGCTCCTTCCCCGAGCTTCCCCGGACAGCGTACTTTGAGGACTCGCTCAGCT
CACCGGGGACTCCCACGGCTCACCCCGGACTTGCACCTTACTTCCCCAACCCGGCCATAGCCTTGGCTTCCCGGCGACCTCAGCGTGGTCACAGGGG
CCCCCCTGTGCCCAGGGAAATGTTTCAGGCTTTCCCCGGAGACTACGACTCCGGCTCCCGGTGCAGCTCCTCACCCTCTGCCGAGTCTCAATATCTG
TCTTCGGTGGACTCCTTCGGCAGTCCACCCACCGCCGCCGCCTCCCAGGTAAGTTTTTGATAGTAGGGGTGCTGCTTTGTAGGTTTTATTTTTTAAG
TCAAGGGTGAAAAGAATAAACCCCAACCCCCACAAAAAGGCGCATCAGAACCCTAGATCTGAGATGGAAAAGGCTCACAGCGCACTTTGCAAACTGC
AAAGAGTCGGGAGATGTTTGCAATTGGTTGCGTGCGTGGAGCGCAAGGAGGGAACGCGGCAGGGAGGGTAGGCTTTGGGGCGAGGTGGGGGTGGGGT
GGGTAATGCGCTGCTCAATGCAACGTGTATGCGGTAGCGGGGCTGAGAACTTTGAGCCGGCCCCGGGACTGCCCCCTGCTCGGGTCCCAGACCTGAA
GCTAGCGCAGTTAGGCAGGTGGGGGAAATCCCGGGGAAGCTTCCAGCAGTCTCTTTTCCTTTCCTCCTCCTTCGGAGCGCCCACTTCGGTGCCGGGT
CGCCCTCCACCCATCGGGAAGAGGGGCCTCGAACCCTCAGCCGCGCTGCCTCCGCCTCCTGCGCGGAGACGTAACGGGGGACCCGTGCGTAACGGCT
GACGCGCTGGAATCCTCCGTCTGACGCGGGGCACGCACGGCGCGCGGCGCCCCCTTCGTCCGCCCCGCCCCTGACGTCCCGGGAGCGTTCTATTTTG
GAACGCCGGGGCCACGTTGCTAAGGGAGGGGGCAGCCCGGCGTTTCGATTGGCCGCCGGGGCGCACGCCTTGGCCAATCAGCTTTCCCTTCCTATTT
GTAGGGTGCATTTTCCTTCCCCCCTCTCTGTCCCCGGAACCCGTGGTTCCTTGGCGGCTGGGTCTCTTTTCGGCGCCTCTAGAGGCAGAGGGAGGGG
ATCCCTGTCGTGACAAGAGCGCCTGTCTGCGACCCAATGGATCTGCGAGGCCCTTGCGGGGATCTAGTCCCTGGGCTCTCAGGAGAAGGGGGTGTCT
GCTTGTGTGCTGGCGTTTCTTGGAGAGATACGGTGGCTGTCACCCTCTTCTCCAGGCACACACAGACACATTCCCACTCCCTCTGCTCCTCATGCCC
GGTTCCTCCGGTGTGTCCCAAGACAGGACTAGAACCGCGAACCGAAGGGCAACCAGCCAGGTGGTCTCCAGGAGCTCCGCCCCCTCTGGGTTCCCAG
GACTCTGATTGGTCGGCGAGCCAGCCCTTCCCTCACCACGCCCCCCGAGAGAGTAGTTAAGCCTTCAGAGCAGTTCCAGGAGTCCATTTACGGGAGG
GGGGAGATGAGCGCTGCTGAGGCTTGGGGGCTCAGGTCCCGCACCATTCCCCCTCCGCGACAATCTGAGAGAGCTCCAGTGGTTACTTTTATCTACC
TGTCCGTTCACCCTAAACTGTCACTCGTCAGTCTCACTCTGAGAAGAGACAGTAACTTGAAACGTTGTTCTAAACTCCTAGGCCCGTCCCCCAAACA
CCCTTTTGACTGGGACCCCCGCCCCTGCATGGCACCTCGCGCAGAGGGGGGTGTGTATGTGTGTGAGTGTAGAGGAAGGCTTGGCCTAAGGCCTCTC
CTTCTCCCTCCCCTTGCCTCTGGGGTGGGGGTGGGGTGTTGTGGCTGTGTGTGTGGCTGTGGCTCCGTCCCGGGGGTTCTGTCACCCGGCTGTGTCC
AGCCTCCTCTCCACCCCCCATACCTAAGAGTCACCAACCCGGGGTGTGATTCACCACCCGCTGGAACCGTGCAACCTTTCCCCGAGGAAGAAGGAGG
AGGTAGAAGCCAGTTGAGCAGAAATCCTCTCATTAACCACTGCGTCACGGTGTAGTGGAAGGGTGGGTGTTGTGGCTTTTTCCCTGTGACACACACA
TCCACACTCGCTCACCCTGTGCTCACTCACGGGGTCGGTGTGTGTTATGTGTGTTGGGTGTGTGTGTGTCGGTGTCTTTGTTTGTGTGTCTACGCCT
GTGTGTGTATGTGTCACCCCGTAGGAGTGCGCCGGTCTCGGGGAAATGCCCGGTTCCTTCGTGCCCACGGTCACCGCGATCACAACCAGCCAGGACC
TCCAGTGGCTTGTGCAACCCACCCTCATCTCTTCCATGGCCCAGTCCCAGGGGCAGCCACTGGCCTCCCAGCCCCCGGTCGTCGACCCCTACGACAT
GCCGGGAACCAGCTACTCCACACCAGGCATGAGTGGCTACAGCAGTGGCGGAGCGAGTGGCAGTGGTGGGCCTTCCACCAGCGGAACTACCAGTGGG
CCTGGGCCTGCCCGCCCAGCCCGAGCCCGGCCTAGGAGACCCCGAGAGGAGACGGTGAGTAAGGGACATCAGAACTTGGCCTGGGTAGGGGGAAGCA
AGAGAGGCAGGAAGTTTCTTATGAATGGAGGGGGGCTCCACTAAGGCCTCAGTGTTACAGAAACCCCAAGATCCTTGCTACACGAGCGAGGACCGGA
GGCTTGTTTTTTGGCTCTTGGGGGTTCTGAAAGAAGTAGAGGTTCGGGATGGGTGGAGGAGTGCTGTATCCCCAAATCTCATGGCCTCTATCTCCCT
GACTCAGCTCACCCCAGAGGAAGAGGAGAAGCGAAGGGTGCGCCGGGAACGAAATAAACTAGCAGCAGCTAAATGCAGGAACCGGCGGAGGGAGCTG
ACCGACCGACTCCAGGCGGTGAGGACAGGCCCTGGGGTGGGAGAGGGGATGTTGAGGGGAGCTCTCTCCCCATTCTCTGCCCCCTCTCCACCTGTAC
CCTTATCCTGGGTTGAGAACTAGACGTTCCACACATGGAACTAGGTACTGCTGTGGCCAGACTGGGTAGCCCAGGGCACAAACACAGACCCCCATGG
ACTTAAGTCAACTCCTGGTCCCCCCCCCATTTCCTGACCCCACGGACTACTCTCCTAGCCTTCATTTCATCCCAAGGGGCCACATGGGGCCCTTGAG
GAGAGGGGCGCCCCCATCATTTCTCCCATCTGGTCTTCAGCAAGTAACAACCCATTTTGCCTCAGTTTCTCCATCTCTGCAAACCCTCATCAAATCT
CCCGGGCTCTTTCTACCCTTAACACTCTGGAAAGCCTGTGAAATGAAATTATTCCACCTCCTGCCCTAGCCACCCACAGCTCTCCTGGTGCTGGTGG
CATCCCCCAAAACCCACTCCCTTCCTACGTCCTCCCTTGGTCTGAGAGTTCCCTGCTGTATGCCTGCAGGGTGAGCTGTTACTCCTTGAGGGAACAA
GGGAATTGTCAACTTTCCTTCTCTACTTTTTCTCTTCCCCGGGAGGTAGAGAGGGAGGGGTAATAGAAGGGAACACATTAAAAACACATAACAGTGG
CTCATGCCTGTGATCCCAGCACTTTGAGAGGCCAAGGCAGGAGGATTGCTTGAGCCCAGGAGTTTGAGACCAGCCTGGGAAACATAGGGAGGACTTG
TCTCTACCAAGAAAAAAAAAAATTAGTTGGGCATGGTGGTGCACACCACTGTGGTCCCAGCTACTATGGAGGCTTTAGTGGGAGGATCGCTTGAGCC
GAGGAGGTCCAGGCTGCAGTGAGCCATGATTGCACTATTGCACTCCAGCCTGGGGGACAGAGCGAGACTCTGGCTCAAAAGCAAAACAAAACCAACC
ACATAACGATTGTTCATTCATTCAACAAACCTCCTGCACACCAGAAACTTCCCCATAAACCTGGCCCCTTCTGTACCATCCTTTGGACAGATGGGGG
AAACTGAGGTTCCTGGAAGGCAAGACCCTTGCCCCAACTCCCATGGCCACCAGGACTCCATCTCAGGAGGTGTTTTTTCTCAGCCCGGGGCTGCCTT
CCAGCAGCAAGTCCAAGGGAGCCATGACCCGAGTTTCCCGGTCACTGACATGCTTTTTTCTCCTTCCTCTCTCTCTTCTGTGACCTGGCCTCCCTGG
CCTCAGGAGACAGATCAGTTGGAGGAAGAAAAAGCAGAGCTGGAGTCGGAGATCGCCGAGCTCCAAAAGGAGAAGGAACGTCTGGAGTTTGTGCTGG
TGGCCCACAAACCGGGCTGCAAGATCCCCTACGAAGAGGGGCCCGGGCCGGGCCCGCTGGCGGAGGTGAGAGATTTGCCGGGCTCAGCACCGGCTAA
GGAAGATGGCTTCAGCTGGCTGCTGCCGCCCCCGCCACCACCGCCCCTGCCCTTCCAGACCAGCCAAGACGCACCCCCCAACCTGACGGCTTCTCTC
TTTACACACAGTGAAGTTCAAGTCCTCGGCGACCCCTTCCCCGTTGTTAACCCTTCGTACACTTCTTCGTTTGTCCTCACCTGCCCGGAGGTCTCCG
CGTTCGCCGGCGCCCAACGCACCAGCGGCAGTGACCAGCCTTCCGATCCCCTGAACTCGCCCTCCCTCCTCGCTCTGTGAACTCTTTAGACACACAA
AACAAACAAACACATGGGGGAGAGAGACTTGGAAGAGGAGGAGGAGGAGGAGAAGGAGGAGAGAGAGGGGAAGAGACAAAGTGGGTGTGTGGCCTCC
CTGGCTCCTCCGTCTGACCCTCTGCGGCCACTGCGCCACTGCCATCGGACAGGAGGATTCCTTGTGTTTTGTCCTGCCTCTTGTTTCTGTGCCCCGG
CGAGGCCGGAGAGCTGGTGACTTTGGGGACAGGGGGTGGGAAGGGGATGGACACCCCCAGCTGACTGTTGGCTCTCTGACGTCAACCCAAGCTCTGG
GGATGGGTGGGGAGGGGGGCGGGTGACGCCCACCTTCGGGCAGTCCTGTGTGAGGATTAAGGGACGGGGGTGGGAGGTAGGCTGTGGGGTGGGCTGG
AGTCCTCTCCAGAGAGGCTCAACAAGGAAAAATGCCACTCCCTACCCAATGTCTCCCACACCCACCCTTTTTTTGGGGTGCCTAGGTTGGTTTCCCC
TGCACTCCCGACCTTAGCTTATTGATCCCACATTTCCATGGTGTGAGATCCTCTTTACTCTGGGCAGAAGTGAGCCCCCCCCTTAAAGGGAATTCGA
TGCCCCCCTAGAATAATCTCATCCCCCCACCCGACTTCTTTTGAAATGTGAACGTCCTTCCTTGACTGTCTAGCCACTCCCTCCCAGAAAAACTGGC
TCTGATTGGAATTTCTGGCCTCCTAAGGCTCCCCACCCCGAAATCAGCCCCCAGCCTTGTTTCTGATGACAGTGTTATCCCAAGACCCTGCCCCCTG
CCAGCCGACCCTCCTGGCCTTCCTCGTTGGGCCGCTCTGATTTCAGGCAGCAGGGGCTGCTGTGATGCCGTCCTGCTGGAGTGATTTATACTGTGAA
ATGAGTTGGCCAGATTGTGGGGTGCAGCTGGGTGGGGCAGCACACCTCTGGGGGGATAATGTCCCCACTCCCGAAAGCCTTTCCTCGGTCTCCCTTC
CGTCCATCCCCCTTCTTCCTCCCCTCAACAGTGAGTTAGACTCAAGGGGGTGACAGAACCGAGAAGGGGGTGACAGTCCTCCATCCACGTGGCCTCT
CTCTCTCTCCTCAGGACCCTCAGCCCTGGCCTTTTTCTTTAAGGTCCCCCGACCAATCCCCAGCCTAGGACGCCAACTTCTCCCACCCCTTGGCCCC
TCACATCCTCTCCAGGAAGGGAGTGAGGGGCTGTGACATTTTTCCGGAGAAGATTTCAGAGCTGAGGCTTTGGTACCCCCAAACCCCCAATATTTTT
GGACTGGCAGACTCAAGGGGCTGGAATCTCATGATTCCATGCCCGAGTCCGCCCATCCCTGACCATGGTTTTGGCTCTCCCACCCCGCCGTTCCCTG
CGCTTCATCTCATGAGGATTTCTTTATGAGGCAAATTTATATTTTTTAATATCGGGGGGTGGACCACGCCGCCCTCCATCCGTGCTGCATGAAAAAC
ATTCCACGTGCCCCTTGTCGCGCGTCTCCCATCCTGATCCCAGACCCATTCCTTAGCTATTTATCCCTTTCCTGGTTTCCGAAAGGCAATTATATCT
ATTATGTATAAGTAAATATATTATATATGGATGTGTGTGTGTGCGTGCGCGTGAGTGTGTGAGCGCTTCTGCAGCCTCGGCCTAGGTCACGTTGGCC
CTCAAAGCGAGCCGTTGAATTGGAAACTGCTTCTAGAAACTCTGGCTCAGCCTGTCTCGGGCTGACCCTTTTCTGATCGTCTCGGCCCCTCTGATTG
TTCCCGATGGTCTCTCTCCCTCTGTCTTTTCTCCTCCGCCTGTGTCCATCTGACCGTTTTCACTTGTCTCCTTTCTGACTGTCCCTGCCAATGCTCC
AGCTGTCGTCTGACTCTGGGTTCGTTGGGGACATGAGATTTTATTTTTTGTGAGTGAGACTGAGGGATCGTAGATTTTTACAATCTGTATCTTTGAC
AATTCTGGGTGCGAGTGTGAGAGTGTGAGCAGGGCTTGCTCCTGCCAACCACAATTCAATGAATCCCCGACCCCCCTACCCCATGCTGTACTTGTGG
TTCTCTTTTTGTATTTTGCATCTGACCCCGGGGGGCTGGGACAGATTGGCAATGGGCCGTCCCCTCTCCCCTTGGTTCTGCACTGTTGCCAATAAAA
AGCTCTTAAAAACGCATTCGCCAGGCTACAGTGTTTATTTCCTCCTAACACCCATTGCCTGGCTTCCTTTTAGTAAGAGAGGATGAGGTTAGATTGT
CGAAGGACTACACACACACAGAGATCATACACCCATGCACACACACACACAGACACACACCTATGCAGGAACTCCTTCTAGTCAATGGGTCTACAGC
AGGATAAAAGGAAGGCAGACAGTCAGGAGGACTTCCTGTACTCGCAGGCAGTGCCTCCTACTAGAAAAAGTAAGTGGCTACAGCTGGGCGCAGTGGC
TTGCGCCTGTAATCCCAGCATTTTGGGAGGCCAAGATGGGCGGATCACTTGAGGTCAGGAGTTTGAGACCAGCCTTGCCAAGATGGTGAAACCCTGT
CTCTACAAAAAATACAAAAATTAGCCTGGTGTGGTGGTGCACACCTGTAATCCCAGGCTACTTGAGAGGTTGAGGCAGGAGAATCACTTTAACTTAG
GAGGCAGAGGTTGCAGTGACACAAGATTGCACCACTGCACTCCAGCCTGGGTGACAAAGTGAAACTGTCAAAAAAAAAAAAAAAAGTAAGGGGTCAG
AAGGTTAGCCTGCAGGTGTGGGATCACAGAGGTCTCCTAGTGATGGAGCTTGTATGCTCTATGGGTTAAAAACAGACGCTAAGGAGATAAACTATAC
ACAGAAGAAGCTTAATGGGCTGTGCACAGTGGCTTGCACTTGCAATCCCAGCTCTTTCGGAGGCCGAGGTAGGAGGCTAGGAGTTCGAGAACAGCCT
GGGGCAACATAGTGAGACACCCCCCACCCCAACCCATCTCATTATGTTGAGAAAAAAAAAAAAAAGAGGATCTTGAGAAACTTGCACAGCAAACTAC
TAAAGACCACTCAGGCTAGGAATGAGGCGTCATCTTGGATTTTTTACAAACTACAACTACAAAAAACAATTATTTTACACTGAAGATGTGTTTCCTT
TTGTTTTCCATATGACCACTATTGGGGCCAACCCAAGACTGACTGAAGCAAATATATGACCATTTTTTTTTTTTACACATAGAGTGGAGCCATCCCC
ATTGCAAAATCCTAGCCGTCTCTTATTTGCATAACAGCAAGTCCTCTGGCAATTGAGGTGAGGGTCCAGGGGTCGCTCAGACGCTTTGGTAAATCAA
TAAGCAAGGGAATTTGTCATGGTGGAATCCATTTATTAATTCACTCAAAAAAATGTGCTAAGCACCTACCCAACTGTGCTGGGAGTTGGGGACACTA
TAGGGACTGAGCCTCAGGTCTTGCCCTCCTGGGGCTCACAGTCCATTGGGGGACACACACCGTTGCCAGTCAGGGACAACCTAAAGTGGATTGGGTT
TGGCAAGCCCATGGGGCTAGGGAAGCACAGCAGGGACACCTAGCTTGGACTGGGAGTCGGGAAAAACTTCCTGGCGGAGGGGACAGCTGAGCTGAGT
CCTAGAGGAGGATAGAGAGTATAGAAAGAAATAGAAAAAGGCCAAGGCCAAGAAAATAGCACAGCCATAGATCTGTTGGTGGGGAGAAGGCTTGATA
CATTGAGAACGCTTAGAGGGTCGGGTGCGATGGCTCATGCCTGTAATCCCAGCACTTTGGGAGGCCGAGGTGGGTGGATCACCTGAGGTCAGGCATT
CAAGACCAGTCTGGCCAACGTGGCGAAACCCCGTCTCTACTAAAAAATATATATATATATATACAAAAAATTAGCTAGGCATGGTGGTGGGCACCTG
TGATCCCAGCTACTCGGGAGGCTGAGGCAGGAGAATCGCTTGAACCTGGAAAGTGGACATTGCAGTGAGCTGAGATTGTGCCACTGCACTCCAGCCT
GGGCAACACAGCGAGACTCTGTCTCAAAAAAAAAAAAAAAAGAAAGAAAAAAAAGAGAAAACTCAGAGATTCGTGGAGACTGGAACCACGGGTGTGG
AGAGAGGGGTTAGTAGAGACCAGATTCTGCAGGTACTATAATGACATTCCCAGGCTAAGGAGTTTAGATCTTTTCTTCAGGGCACTGGGGAGCTATT
GCAGGTTTTGAACACGAGAGGGGCAGGGGCAGGTTTGTGATTTAGGAAAGACCCCTCTGGCCCCAGACTGAGCGTGACTGGAAGGGAGAGGAGTGAG
ACTGAGCAGGAGACCAGGGAGGAAGTGGGGTGGGGACCCTGCAGGGTGGGGTGAGGAGGAGTCCAGAACAGGGCCAGGGACATGGGACAGAGAGGAG
GGGAAGGATTCAGGAGACACTCGGGAGGCAGGACAGTTGGGCTTGGTGAGCAGCCGGCTTGGGAAGGAAAATACAGGGCAGTAATAAATAGCATGGG
TCAAGTGGTCTGAGTGAGGCTGGGAGTGGTGGCACGCACCTATGGTCCCAGCTACTCAGGAGGCTGAGGTGGGAGGATTGCTTGAGCCTGGAAGTTT
GAGGCTGCAGTGAGCTATGATCGTACCGCTGCACTCCACTTTCGGTGACAGAGTAAGACCCTGTCTCAAAAAAAAAAAAAAAAAGGAAAAAAAAAAA
AAGACACTGCCTATGAGTTAACCCTGCTCTACAAGGAGCAGTTTTTAAAGTAAAATTAAAAAAAAAAAAAAAAAAGAAAGAAAGAAAGAAAGAAAGA
GAGAGAAAAAAGAAAAAAGGCTGGGTTGCCTTGGACAACAGACTTCATCTCCCTGAGCCTCCATTTCCTCATCTGTAGAATGGGGGCTGTTAAGAGG
AGTTGCAAGGCTTGTGCATGCCAGCAGTAAGTGCAGAGTGACGGTGCAATTATCATTACCCCCATCATCTTTATTGGGGTCAGCCTGAACCCTCGAT
ATCCCATAATATTCACCCCCATCCTTCAAGGGTCTGCCCTAATGTTCCCATGACACCCGACCAGCTCAGCTCTCCTTATGAGAGGGCCTCACTTTTC
TATTCCCTTTGCAGCCGTTATCCCCTTTGTAGTTGTTAATTAAGTGTGTAATTACATGATGGCTTAATGTTTGTCTCCCCCACTGGGCTGACTGCAC
CAGGAGACCAAGGCCAGGGCTGTCACCCACCGCTGCGTCCTCAGCAAATGCTTGTTTGATGAGTGAATGACAGATGAACAAATGGGCAAGTATTTGA
CTGATTAATCACGGCATGCGTGATTAAATAAATGAGTGAGCAAACGACTGAATGAGTAAGCAATTGAAGGGGAGAGATCTAGGATAATTTCCAAACT
GCCAATATCCCAGAACTGGGTAGATGACTTTTTCTCCGTCTTTTGGAGTGGGTTTTTATACTCCGGAGGGGGAAATAAGTAACGACAATCAGAGATC
GGAGTGACAGAGAATGAGTTTGCGATGCGGAAGGCCGCAGCGACCTCTCCAGGTGCAGGCTGGAGGAGCGCCTCCCGGGCAACAAGCAAAATAAGTC
CGGTTCGATAAGTAAGATTCAAGGCGCTTAGTTACTACCGCCCGAAAGGTGGAATTGAACCACTCTGTCGCTAGACAGCTACAGGTTTGAAGCCTGC
ACCCCAGACCACTGAGGATCATCCGGGCAGGACAACCTTCTTCCCGCGCAGCTATATAAGGATCGCAAAAGCTTACCTTTTAGAGTTATGGTCGCGT
CCTTTGGAAAGGTGTGAGATGGTTGACTTCAAGGGCTATAAGTTCTCCCCACTCATTTATCGTGAGAGATGTCCTTGTAAAACGTTGCCTTCACTTC
ATATAGTATGAATCATCCTCGCTCCCCAAACCGTCTCATTTACATAGCGCCACGCATTCTGGGAAGCAGCGTTTGAGCACCTGGCAAGCTGTTAAAG
GGCCCAGGACCCCCTTTTTCCAAAATGAAAAGTCAGCTTTTTAAGAGGAGAGATTCATGATTCGGCATTCAGAAGCGCCTTCTCGGAAAATTCCGAA
TTCTCTTTCTGCGGAGGAGTGGTGAGTGGGTTCCTGTTAGAGTCTGATTCACCCTGGCCCCAAAGGCGCTGAGCAGGCGGCTATAGGGCTGGTGCGA
CACCTGCTGTTCTTTTATGGAATTGCGTAGGCCGATGGAGCTCCGCGTTCATTCAAACGTTTATTGAGCGTCTACTGAGTCCCAGGAACTGTCTCGG
TGCTGGTGACGACAGACATCCTGTCCCGATTGAGTTTAAACACTAGACATAACCAATTTCTGCACTTGCGTTTTATGGTTGGAGAAACTGAGACAGA
CTCAGAAACGGATAGTACCATATTCCGGTTAAAACAGCCCATCCACTTAGGGAAATCGAACTTGTGTTACAATTTTTATTACAAAAAAGGTGATTCT
TTTTTCTTAAATTTTTACATATTTAGGAGGTACAGGTGCCGATTTCTACATGCATATGTTGATTGCATCGTGGTGAAATCTGGGCTTTTAGGTGTAC
TCATCACGAAAAATTATTCAATATTCTAAGTCAGTGATTCTTTGACAATACCATTCTCCTGCATGAAACTCTCCCATGACTTCCATTTCAGAGTAAA
AGTCAAAGTACTCACCCTGCTTTACAAATCCCTGTAATCCAGCCCCTTTGGTCCCTCTGACCCTGTCTCACATACTCCTCTTCCCTTGGATATTATT
GGATTTAGGCACACAGATGTCTTGCTGCACACATGGATCATGCTACCACCACAGGGACTTTGTGCTTGCCGATCCCCTAGCCTGGATCTCCTCTTCA
CATGTTCCCATGGCTCATCGCCTCCCTTCACTCTGTCATCTGATCAAATGTCAATACCAAAAACTCACTGAGTGACCAGCACTCAGTGGGGCCTTCC
CTGATCCCAGTTTAAAATAGCGAGTGGCGGCCGGGCGCGGTAGCTCACGCCTGTAACCCCAGCACTTTGGGATGCCAAAGCGGGTGGATCACGAGAT
CAGGAGATGGAGACCATCCTGGCTAACACGGTGAAACCTTGTCTCTACTAAAAATACAAAACATTAGCTGGGCGTGGTGGCGGGCGCCTGTAGTCCC
AGCTACTCGGGAGCCTGAGGCAGGAGAATGGAGTGAACCCGGGAGGCGGAGCTTGCAGTGAGCTGAGATTGCTCCACTGCACTCCAGCCTGGGGGAC
AGAGCGAGGCTCCGTCTCAATAAATAATAAATAAATAAAATAGCTAGTGGCTGGGATCGGTGGCTCAGGGCCTGTAATCCCAGAACTTTGGGAGGCC
GAGGCGGGTGGATCACCTGAGGTCAGGAGTTCGAGGCCAGCCTGGCCAACATGGTGAAACCCCGTCTCCACGAATGATACAAAAATTAGTCAGGCAT
AGTGGCTCACGCCTGTAATCCCAGCTACTCGGGAGTTTGAGGCAGGAGAACCGCTTGAACCGGGGAGGCAGAGGTTGCAGTGAGCTGAGATGGTGCC
ATTGTACTCCAGCCTTGGTGAAAAGAGCAAAACTCCATGTCCAAATAAAAGTAATAATAATAATAAATAAAATAAAAATTTTTAAAAAGCGAGCTCT
GTCACTCTATCCCCTTACCCTGCTAGACATTTCCCCACCACTCTGATCACCACCTGCCATTTTCTGTGTCTATTTGCTTACCGTCTGTCTGCTTCTG
TAGAATATCAGCACCATGAAACTATGCACTTTATTTTTGATCATTGCTGTATCTCTAGTGCCTAAAAAGTGCTTGAGAACATAGCAGATGTTCAGTA
AATGTTTGTGGAATGAATAAAAGAATATCATCACTGTCTTTTTTTCATTCTTCTTCCAGACAGGATCTTACTTTGTCACCCAGGCTGGAATGCAGTG
GCGCAAAAACGGCTCACTGCAGCCTCGACCTCCCAAGCTCAAGTGATCCTCTTGCCTCAGCCTCCCTGGGATGACAGGCATATACCATCACGCCCAG
CTAATTTTAATTTTTTTTGTAGAAACAGGGGCCTCACTTTGTTGCCCAGACTGGCCTCGAACTCCTGGCCTCAAGTGATGCTTCTGCCTTGGGCTCC
TAAAGTGCTGGAATTTCACGCGTGAGCCACAGCACCTGGCCTCACTATCCTTCTTTCAGCCTCAGTTTTCTCATTTGTATAACCAGACTAGTACAAC
TGATCTCACTGGAGAAATCATGATATAAAATTCTGACACTGGCTGAGGCAACTGGGAGGAGCTCAGTAAAGGCTGTTTCTGCTGGGCACGGTGGCTC
ACACATGTAATCCCAGCACTTTGGGAGGCCGAGGTGGGTGGATCACAGGAGATTAGAAGTTCCAGACCATCTGGCAAGCATGGTAAAACCCCATCTC
TACTAACAATTCAAAAAGTAGCCAGGCATGGTGGCTCACACCTGTGATCCCAGCTACTCGGGAGGCTAAGGCAGGAGAATCCCTTGAACCCAGGAGG
CTGAGGTTGCAGTGAGCCAAGATTGTGCCACTGCACTCCATCCTGGGCGACAGAGCAAGACTCTGTCAAAAAAAAAAAAAAAGTTTTTTTTTTTTGG
CTGGAATTACAGGCGCCTGCCCCCACACCTGGCTAATTTTTGTTTTTTGTTTTTTTAGGAGAGACGGGGTTTCACCATGTTCACCAGACTGGTCTTG
AACTCCTGACCTCAGGTAATCCAACTGCCTCAGCTTCCCAAAGTGCTGAGATTACAGGCGGGAGCCACTACACCTGGCCAATAAAGGCCGTTTCAGT
CTTCAATCTGTTTTGAGCTTGGAGGCTTTAGTCATTCCGAGACCCAAAATCTCAATCAGACCCTCTTCCACCACTTTTTGTGATAGATCAATAAACA
TTTTGTCTTATGGGAAGTTTAACTAAGAGTATCTTTAGAAAGTTTTGGACAGGCGCTGTAATCCCAGCACTTTGGGAGGCCGAGATGAGCGGATAGC
TTGAGCCCAGGAGTTAGAGACAAGCCTGGGCAACATAGTGAGACTCTGTCTCAAAAAAAAAAAAAAGAAAGAAAGGAAAAGAAAAAAAGAAAAAAAA
AGTATTCCCTCTGCTTGGCAAATCCAGATTCAAGATATATCCCCTAAACCCTCTTTGTTTTAATTAGATAGTTGCTGCTAGGCACCTCTGTATACAA
ATTCTGAGGATGTAAGGACTCCTTGGAACACCGTTATCTGTCTCCTAATATGTGACGTGTGTATGACGAACAAGTGAGTTTTTTTTTTTTTTGTTTC
TTTTTTTTGGTTTTTTGTTTTGAGACGCAGTCTCGCTCTGTCGCCCAGGCTGGAGTGTAGTGGCGCGATCTTGGCTCACTGCAAGCTCTGCCTCCCA
GGTTCACGCCATTCTCCTGCCTCAGCCTCCCTAGTAGCTGGAACTACAGGCGCCCGCCACCACTCCCGGCTAATTTTTTTGTATTTTTAGTAGACAC
GGGGTTTCACCGTGTTAGCCAGGATGGTCTCGATCTCCTGACCTCATGATCTGCCCGCCTCGGCCTCCCAAAGTGCTGGGATTACAGGCGTAAGCCA
CCGAGCCCGGCTTTTTTTTGAGATGGAGTTTCCCTCTTGTTGCTCAGGCTGGAGTGCAGTGGTGTGATCTCGGCTCACTGCAACTTCCGCCTCCCGG
GTTCAAGCGATTTTCCTGCCTCAGCCTCCCAAGTAGCTGGGATTACAGGCATGCACCACCACGCCCGGCCAATTTTGTATTTTTAGTAGAGACATGG
TTTCTCCATGTTGGTCAGGCTGGTCTCCAACTCCCGACCTCAGGTGATCCACCTGCCTTGGCCTCCTAAAGTGCTGAGATTACAGGCGTGAGCCACT
GCGCCTGGCTGCACAAACATTTTAAACGTCAATATTTTTGTGTTTATTTTTACTATTGTCTTCTATTTCTGGCAAGCAATACTGGTTTCTGGTGGCA
AGGTAACCTTTCTTCTTAAAATACATTTGTTATTATATTATTAAATATTAAACCAATTTTCAAGAAAAATATTAGATAAATAGCAGAACAGGTTGTA
GAAAGATGAAGGTCATGTGGGGAATGACTGAGGTTTGGGAAACAATATTATAACTCCTTACTGTGCATTTATTATATGCCAGGCCCAATGCCAAGGG
CTTTACATGTAGGTATAGTTATGGTTGACAGTTTTCTTCTTTTTCTTTTCTTTTTTTTTGAAACAGGGTCTTGCTCTGTCGCCCTGTCTGGAGTGCA
GTGACGCAATCTCAGCTCACTACAACCTCCACCTCCTTAGTTCAAGTGATTCTCCTGCCTCAGCCTCCCGTGTAGCTGGTCTTACAGGTGCCCACCA
CCACATCCGGTATTTTTAGTAGGGACGAGGTTTTACCATGTTGGCGAGGCTTGTCTTGAACTCCTGACCTCAGGTGATCCACCTTCCTTGGCCTCCC
AAAGGCTGGGATTACAGGTGTGAGCCACTGCACCTGGCCTCTTTTTTTCTTACTTAATTTTTTTGTAGAGATGAGGTCTATGTTGCCCAAACTTGTC
TCGAACTCCTGGCCTTAAGTGATCCTCCCTCCTTGGCCTTCCAAAGTGCTGGGATTGCAGACGTGAGCCATCGTGCCCGGCCTAATAAGTTTTCCAT
TTGAGGAAACTGAGGCTTTGAGAGATGAAGTCATATGCTCAAGGTCATACAGCGAAGGGGTAGTAGAGCCAGGATTCAAACCCAAGTCTGTGGATCT
TTAGACCTCAGTGTTCTCCCAGTCTCCCCACGGGTCCCTTTGCTTCATCCTTTCCGAGTTTAGTTGTCTTTTTTGTTGTTGTTCTTGTTGTTGCTGT
TTTTTGATAGAGTCTCGCCCTGTTGCCCAGGCTGGAGTGCAGCGGCATGATCCTGGCTCACTGCAACCTCTGCCTCCTGGGTTCAAGTGATTCTCCT
GCCTCAGGCTCCCGAGTAGCTGGGATTACAGGTATGCGCCGCCACACCCAGCTAATTTTTGTATTTTTAGTAGAGATGGAGTTTCATCATGTTGGTC
AGGCTGGTCTGGAACTCCTGACCTCAGTGATCTGCCCACCTTGGCCTCCCAAAGTGCTGGGATTACAGGCGTGAGCCACTACGCCCAGTATGTAGTT
TTTTTGTGTTTTGTTTTTGTTTTTGTTTAGAGACAGGGTCTCCCTCTGTAACTCAGGCTGAAGTGCAGTGGTGTGATCATAGCTCTCTGTAACCTCG
AACTCCTGGCCTCAAGCAATTTTCCTGTCTTAGCCTCCCAAGTACCTGGGACTACAGGCATGCACCACCACGCCCGGATAAATTTTTTTTTTTTTTA
GAGACGGGGGTCTTGTTATGTTGCTCAGGGTGGTCTCGAACTTTTGGCCTCAAGTGATCTTCCCACATTGGCCTCCCGAAGTGTTGGGATCACAGAT
GTGAGTCATCATGCCAGGCCTCAGATTAGTGTTGAAACTGGAAGTCCAGGGAGGCCCTGTATTTGTTCCCGAGGCAATCAGGGTGGGGAGACGTGGG
TGGATGAGAGAAGTTTGTGAGGCAGGATAGACACAGCATTGTGACTGATTGTTTGTGGGAGGTGAGTGTGTAGAAGTCCAGAGCCATACCTGGGTGT
CTGGCCTGGTGACACCGTGGGCAGTGAAAACATCTCTGAGTTGGAAATCAGGTAGGATGAAGAAGACGTTTGGTTAGATTTAGGGTGGAAGGGGCAT
CCAGAGGCCGTTGGACACGTGGGTTTGGGCTCAGTTGAGGTGGCTGCACAGTAGGCAGGGTGTGGGATCCATTAGTGATGTCTGCCTTGAGCACAGG
CATGGGAAGTGGCTGCCAAGGCTTGGCTAACCCTGGAATGGGTGGTCTGTCAGCATCTTCCCCTCAATCATTTCTTTCTTTTTTTTTTTTTTGAGAC
AGAATCTCGCTCTGTCGCCGAGGCTGGAGTGCAGTGGCGCAATCTTGGCTCACTACAACCTCCATCTCCCAGATTCAAGCGACTCTCATGCCTCAGC
CTCCCGAGTAGCTGTGATTACAGGCATGCACCACCACACCCGGCTAAATTTTGTATTTTTAGTAGAGATGAGGTTTCACCATGTTGGCCAGGCTGGT
CTTGAACTTCTGGTCTCAAGTGATCCGCCCTCCTCGGCCTCCCAAAGTGCTGGGATTACAGGCGTGAGCCACTGCGCCCGGCCTCCCCTCAATCATT
TCATAACCTACACAGATGGCAGAG
HUMAN SEQUENCE - mRNA
CATTCATAAGACTCAGAGCTACGGCCACGGCAGGGACACGCGGAACCAAGACTTGGAAACTTGATTGTTGTGGTTCTTCTTGGGGGTTATGAAATTT
CATTAATCTTTTTTTTTTCCGGGGAGAAAGTTTTTGGAAAGATTCTTCCAGATATTTCTTCATTTTCTTTTGGAGGACCGACTTACTTTTTTTGGTC
TTCTTTATTACTCCCCTCCCCCCGTGGGACCCGCCGGACGCGTGGAGGAGACCGTAGCTGAAGCTGATTCTGTACAGCGGGACAGCGCTTTCTGCCC
CTGGGGGAGCAACCCCTCCCTCGCCCCTGGGTCCTACGGAGCCTGCACTTTCAAGAGGTACAGCGGCATCCTGTGGGGGCCTGGGCACCGCAGGAAG
ACTGCACAGAAACTTTGCCATTGTTGGAACGGGACGTTGCTCCTTCCCCGAGCTTCCCCGGACAGCGTACTTTGAGGACTCGCTCAGCTCACCGGGG
ACTCCCACGGCTCACCCCGGACTTGCACCTTACTTCCCCAACCCGGCCATAGCCTTGGCTTCCCGGCGACCTCAGCGTGGTCACAGGGGCCCCCCTG
TGCCCAGGGAAATGTTTCAGGCTTTCCCCGGAGACTACGACTCCGGCTCCCGGTGCAGCTCCTCACCCTCTGCCGAGTCTCAATATCTGTCTTCGGT
GGACTCCTTCGGCAGTCCACCCACCGCCGCGGCCTCCCAGGAGTGCGCCGGTCTCGGGGAAATGCCCGGTTCCTTCGTGCCCACGGTCACCGCGATC
ACAACCAGCCAGGACCTCCAGTGGCTTGTGCAACCCACCCTCATCTCTTCCATGGCCCAGTCCCAGGGGCAGCCACTGGCCTCCCAGCCCCCGGTCG
TCGACCCCTACGACATGCCGGGAACCAGCTACTCCACACCAGGCATGAGTGGCTACAGCAGTGGCGGAGCGAGTGGCAGTGGTGGGCCTTCCACCAG
CGGAACTACCAGTGGGCCTGGGCCTGCCCGCCCAGCCCGAGCCCGGCCTAGGAGACCCCGAGAGGAGACGCTCACCCCAGAGGAAGAGGAGAAGCGA
AGGGTGCGCCGGGAACGAAATAAACTAGCAGCAGCTAAATGCAGGAACCGGCGGAGGGAGCTGACCGACCGACTCCAGGCGGAGACAGATCAGTTGG
AGGAAGAAAAAGCAGAGCTGGAGTCGGAGATCGCCGAGCTCCAAAAGGAGAAGGAACGTCTGGAGTTTGTGCTGGTGGCCCACAAACCGGGCTGCAA
GATCCCCTACGAAGAGGGGCCCGGGCCGGGCCCGCTGGCGGAGGTGAGAGATTTGCCGGGCTCAGCACCGGCTAAGGAAGATGGCTTCAGCTGGCTG
CTGCCGCCCCCGCCACCACCGCCCCTGCCCTTCCAGACCAGCCAAGACGCACCCCCCAACCTGACGGCTTCTCTCTTTACACACAGTGAAGTTCAAG
TCCTCGGCGACCCCTTCCCCGTTGTTAACCCTTCGTACACTTCTTCGTTTGTCCTCACCTGCCCGGAGGTCTCCGCGTTCGCCGGCGCCCAACGCAC
CAGCGGCAGTGACCAGCCTTCCGATCCCCTGAACTCGCCCTCCCTCCTCGCTCGGTGAACTCTTTAGACACACAAAACAAACAAACACATGGGGGAG
AGAGACTTGGAAGAGGAGGAGGAGGAGGAGAAGGAGGAGAGAGAGGGGAAGAGACAAAGTGGGTGTGTGGCCTCCCTGGCTCCTCCGTCTGACCCTC
TGCGGCCACTGCGCCACTGCCATCGGACAGGAGGATTCCTTGTGTTTTGTCCTGCCTCTTGTTTCTGTGCCCCGGCGAGGCCGGAGAGCTGGTGACT
TTGGGGACAGGGGGTGGGAAGGGGATGGACACCCCCAGCTGACTGTTGGCTCTCTGACGTCAACCCAAGCTCTGGGGATGGGTGGGGAGGGGGGCGG
GTGACGCCCACCTTCGGGCAGTCCTGTGTGAGGATGAAGGGACGGGGGTGGGAGGTAGGCTGTGGGGTGGGCTGGAGTCCTCTCCAGAGAGGCTCAA
CAAGGAAAAATGCCACTCCCTACCCAATGTCTCCCACACCCACCCTTTTTTTGGGGTGCCCAGGTTGGTTTCCCCTGCACTCCCGACCTTAGCTTAT
TGATCCCACATTTCCATGGTGTGAGATCCTCTTTACTCTGGGCAGAAGTGAGCCCCCCCTTAAAGGGAATTCGATGCCCCCCTAGAATAATCTCATC
CCCCCACCCGACTTCTTTTGAAATGTGAACGTCCTTCCTTGACTGTCTAGCCACTCCCTCCCAGAAAAACTGGCTCTGATTGGAATTTCTGGCCTCC
TAAGGCTCCCCACCCCGAAATCAGCCCCCAGCCTTGTTTCTGATGACAGTGTTATCCCAAGACCCTGCCCCCTGCCAGCCGACCCTCCTGGCCTTCC
TCGTTGGGCCGCTCTGATTTCAGGCAGCAGGGGCTGCTGTGATGCCGTCCTGCTGGAGTGATTTATACTGTGAAATGAGTTGGCCAGATTGTGGGGT
GCAGCTGGGTGGGGCAGCACACCTCTGGGGGGATAATGTCCCCACTCCCGAAAGCCTTTCCTCGGTCTCCCTTCCGTCCATCCCCCTTCTTCCTCCC
CTCAACAGTGAGTTAGACTCAAGGGGGTGACAGAACCGAGAAGGGGGTGACAGTCCTCCATCCACGTGGCCTCTCTCTCTCTCCTCAGGACCCTCAG
CCCTGGCCTTTTTCTTTAAGGTCCCCCGACCAATCCCCAGCCTAGGACGCCAACTTCTCCCACCCCTTGGCCCCTCACATCCTCTCCAGGAAGGCAG
TGAGGGGCTGTGACATTTTTCCGGAGAAGATTTCAGAGCTGAGGCTTTGGTACCCCCAAACCCCCAATATTTTTGGACTGGCAGACTCAAGGGGCTG
GAATCTCATGATTCCATGCCCGAGTCCGCCCATCCCTGACCATGGTTTTGGCTCTCCCACCCCGCCGTTCCCTGCGCTTCATCTCATGAGGATTTCT
TTATGAGGCAAATTTATATTTTTTAATATCGGGGGGTGGACCACGCCGCCCTCCATCCGTGCTGCATGAAAAACATTCCACGTGCCCCTTGTCGCGC
GTCTCCCATCCTGATCCCAGACCCATTCCTTAGCTATTTATCCCTTTCCTGGTTTCCGAAAGGCAATTATATCTATTATGTATAAGTAAATATATTA
TATATGGATGTGTGTGTGTGCGTGCGCGTGAGTGTGTGAGCGCTTCTGCAGCCTCGGCCTAGGTCACGTTGGCCCTCAAAGCGAGCCGTTGAATTGG
AAACTGCTTCTAGAAACTCTGGCTCAGCCTGTCTCGGGCTGACCCTTTTCTGATCGTCTCGGCCCCTCTGATTGTTCCCGATGGTCTCTCTCCCTCT
GTCTTTTCTCCTCCGCCTGTGTCCATCTGACCGTTTTCACTTGTCTCCTTTCTGACTGTCCCTGCCAATGCTCCAGCTGTCGTCTGACTCTGGGTTC
GTTGGGGACATGAGATTTTATTTTTTGTGAGTGAGACTGAGGGATCGTAGATTTTTACAATCTGTATCTTTGACAATTCTGGGTGCGAGTGTGAGAG
TGTGAGCAGGGCTTGCTCCTGCCAACCACAATTCAATGAATCCCCGACCCCCCTACCCCATGCTGTACTTGTGGTTCTCTTTTTGTATTTTGCATCT
GACCCCGGGGGGCTGGGACAGATTGGCAATGGGCCGTCCCCTCTCCCCTTGGTTCTGCACTGTTGCCAATAAAAAGCTCTTAAAAACGC
HUMAN SEQUENCE - CODING
ATGTTTCAGGCTTTCCCCGGAGACTACGACTCCGGCTCCCGGTGCAGCTCCTCACCCTCTGCCGAGTCTCAATATCTGTCTTCGGTGGACTCCTTCG
GCAGTCCACCCACCGCCGCGGCCTCCCAGGAGTGCGCCGGTCTCGGGGAAATGCCCGGTTCCTTCGTGCCCACGGTCACCGCGATCACAACCAGCCA
GGACCTCCAGTGGCTTGTGCAACCCACCCTCATCTCTTCCATGGCCCAGTCCCAGGGGCAGCCACTGGCCTCCCAGCCCCCGGTCGTCGACCCCTAC
GACATGCCGGGAACCAGCTACTCCACACCAGGCATGAGTGGCTACAGCAGTGGCGGAGCGAGTGGCAGTGGTGGGCCTTCCACCAGCGGAACTACCA
GTGGGCCTGGGCCTGCCCGCCCAGCCCGAGCCCGGCCTAGGAGACCCCGAGAGGAGACGCTCACCCCAGAGGAAGAGGAGAAGCGAAGGGTGCGCCG
GGAACGAAATAAACTAGCAGCAGCTAAATGCAGGAACCGGCGGAGGGAGCTGACCGACCGACTCCAGGCGGAGACAGATCAGTTGGAGGAAGAAAAA
GCAGAGCTGGAGTCGGAGATCGCCGAGCTCCAAAAGGAGAAGGAACGTCTGGAGTTTGTGCTGGTGGCCCACAAACCGGGCTGCAAGATCCCCTACG
AAGAGGGGCCCGGGCCGGGCCCGCTGGCGGAGGTGAGAGATTTGCCGGGCTCAGCACCGGCTAAGGAAGATGGCTTCAGCTGGCTGCTGCCGCCCCC
GCCACCACCGCCCCTGCCCTTCCAGACCAGCCAAGACGCACCCCCCAACCTGACGGCTTCTCTCTTTACACACAGTGAAGTTCAAGTCCTCGGCGAC
CCCTTCCCCGTTGTTAACCCTTCGTACACTTCTTCGTTTGTCCTCACCTGCCCGGAGGTCTCCGCGTTCGCCGGCGCCCAACGCACCAGCGGCAGTG
ACCAGCCTTCCGATCCCCTGAACTCGCCCTCCCTCCTCGCTCGGTGA

TABLE IV

MOUSE NOMENCLATURE
ICSGNM Cmkbr7
Celera mCG1536/2
HUMAN NOMENCLATURE
HGNC CCR7
Celera hCG1E43840
MOUSE SEQUENCE—GENOMIC
NNNNNNNNNNNNNNNNNNNNNNNNNNNNNNNNNNNNNNNNNNGGGAGTTCCATTCTCAGCCCGCTGCTACTGCCTGTGAGCCTCCTTTGGGAAAGTC
CTTAATTTCTCTAGTTCTGTTATTTCCTCATCTCTAGAATGGGCATCACGACACTGATTTCACAGAGTTGTTCCGAGATGAAATGATGTGATGGAGT
AAGTACTAGTTCCCAGCCTCTCAGATCCTGGAAATGCGTGTCCTGCCTCTCCCTCTGGAAGCTCCCTGAAGGCCAGGCTTTGCGTCCTTCATCCAAG
CTTGGATGAGTATTTTAAAAGAGTCTCCGTGAGTTTTGCTCATTAGCCAAAACGGATGCTGGCCTTTGGAAGCGGGGCGGATGACTCTGCGTGGAAA
TATTCTTCTCTGAGTTTCCCTCCTCTGCCTACTTCCTCGTGAGTTCTATGTGGCATCTATTTGAGGTCAAATCTATTGAAATATACTTCAGCTTAAC
TCTAGGTGCCTTTTAGTTGAGCACCAAGAATATACTAAGTGCTATGTAAGAAATAGATAAAGGACCCAGTATGGAGGAGGAGAAATACCAAGCCAAG
CCTAGTACTCAAACCGCAGACAGTGATAGGGACAGCCGGATCGCTTTCTGTAGGCTTGAGGGTTTCTGAAATTCTTCAGGGAATTGGGAGCTCTTTG
AGCTGGACTTTTTTAAGCGTATGAGTGTTTGGTCTCTCTCTATGCATCTGTGTCATGAGCATACCTGGTACCTATGCAGAACAGGAGAGGGTATCAG
ACACCCTGGAACTACAGATAGTTGTCAGTTGCCATGTGGGTGCTGGGAAACCCACCCTAGGTCCTCTGCAGGAACAGCAAGTGCTGAAGGGACTACT
GAGCTGTCTCTCCAGCCCCTTACGTTAAATCTTGAAGAACGGGTTAAAAGATGACAGAGCGGGGGAGAGGTACCGGAGAGAAAAATGTGTGAAAGTA
GTCAGGGCCTTGACCTGGAAGAGTTAGAGAACAAGGCAGATGCAGTTGGCAGGTCCGGAGCAACAGCTCCTGAGTGTTCAATGGGCCAAGTTGAGAG
GGGCTTTGGATGCCATGAAAGGTTGCCGAGGAGAAGGAAGTAGAGAGTGAGTCCTTCAGCTACCACAGGGGAAGGGTGGGAAGGGGGTGAGGAGATT
TCCTAAATGTGCATCATTGGCCTTCTGAGGTCTTCAGAAGACGACCAGTGGGGTCCCCAAAGGGTGAGACCAGACAGAACAGACAGCCACTATTCCC
TACTCTGACCCCCTACCTCAAGAGGAAAGGACGAAGAGAGAGCTGAGAAGAGACCCTGAGCCTCTTCACCACAGCAGACAGGACCAAGTTTCAGCAG
GCACCAGGACACTCAGGTGTCTACGTCAGTCGCTAGCTATGCACATGGGTACCCTGGGCATGGAAACTACAGACTGGCTTTCTAACACATCATTTTG
CCGTGAGACCAATCCCAGCATTCCCTCTATTGTCCTCAACAGAATCAAACGTCCAAGCTAAGGGGCTTGCCACCTCTCCAAAATAAAGGTCACCTTA
GAGGTCAAGAAAGCATTGACACAAACCTTGGGTAAGCCCTAAGACTGTAACTTGGGAGTTGTCATGACAACAGGGCAGTAGAGCCATCTCTACTCTC
TCCTAAATGGCACGAGGTTTAGATGAGTAACTCTGAAACAACAGACCCCACCCCTGTTTTCCTATTTACCATCTTCAGACCCCTCCTCACCTGTGCC
CCAGCCCTCTCCTCTAGGACCTTAGCAACTTGAACAACATAGACACACACGCCTCCTCTCAGTGTCCAACAATGAGCCCTCCCAGCAAAACAGGAAG
CCTCTTCCTGCCAAGGCATTGTGCTTGCCCTGTCACGTGTCAGGAAACCACTACCGTGCCATGGCAACAGGTGTTGCTGAGACCTTAGCCTTCCCGC
CAACCTGTGCCCTGCCCTTCTCTCTCCTCCTTACTCCTAGAGAGTGTCCTCGTTGTGCAGTCTCCTTGACTCACCTGGACACTGCTGTGAGTGAGCG
TCCTAATTACCTTAACTTGGATGAGAAGACTAATTCACCCTATAAGTCAGTGGCCCAGGGCCAGGGCTTGGCCCTTAGACTGCATAAAAAGGAGAAA
GCTGCCGACTGCAGGTGTTCATCTCCCCTTGTTTCCTCCCTATCAGTGCAATGTCACCAGCCACTCCAAGCCAATGTCACCACCAGCCACCCCAAG
CTCCTGCTGCCATGATTCCCCAACCAGGATGGACTCTACGCTGGAACTGTGAGCTGTATTAACGCTTCTCCCCTAAATTTTTCTTGTCAAGGTGTTT
TCATCACAACAGCAGAAAAACCAAAACCAACTGACACTTCCTCCTTGCAACTGGCAGCTCACATCGCTTGCTCCTTTCCCATTTGGACACCAAAGAT
CTCACTGATCATCATCCAAACACAGTTCTTCATGCTAGCCAGGCAACATAAGAATGTACCTGTGTGGAAATTTAGCAAGCTTCTTAGCTCTTTTGGA
TCGCAAAAGTCTAGGTAAGGTCTGAGATGACTCTCTCTCAAATTCCAATATCCCTAAATGTATGCCAGACCTTCCTGTAAGGTTGCTGAGTCCTGAA
GCTAAACTGACTGTCTTAGTTAAGGTTAGTAGTGCTACCACACCAAAAACAAAACAAACAAACAAAAAAAAAATGAACAAGCCAGGCGTGGTGGTGC
ATGCCTTTAATTCCAGCACTCGGGAGGCAGAGGCAAGCAGATTTCTGAGTTCAAGACCAGCCTGGTCTACAAAGTGAGTTCCAGGACAGCCAGGGCT
ACACAGAGAAACCCTGTCTCAAAAAAACAAACAAACAAACAAATAACAGCAACAACAACAAAAACATCAACAAAAGCTATTTGGTTGGGGAAGGTTT
TATTGGCTCACACTTCCATATCATGGTCCATTATCAAAGGAAGTCAGGGCAGGCACTCAAGGCAAGGCAGGAACCTAGAGGCAGGAGCTGATGCTGA
GGCCATGGAAGAGTGTTGCTTACrGGCTTGCTGAGTCTGCTTTCTTTTATCATCCATTGTTACATGGAGATTAATCAAAGTTGCATGAAAAGAAGAG
AGAAGGACCTCCTCAGACGCAGATGGAAGCATGTGGTCCTTTGTTAGGACTCTGAAGGGCAGTGGTCTCTCAGAAAAAGGCTGTCAGCCAAGGCACA
GGGGGTTTGAGGGTTTTATAGGACAGAAAAGGAAGCGTTTGTGGTTGGAAAATTCCATCTGCAGATCTGTTTCCTCAAGTCTCAGCACTGTAAATAC
CAACGGAGGTTCTTATCCATTAGTGAATTTCTTGTCAGGAGAATCCCGGCTCCTAACTGCCTGCAGATGTTAGCAGTGTATCCCTGGTGCCATCCGG
AGTTTCTCAGCTCTCCTGGGGCTTTTAGGTCATTAACCCTTCACCCAAGACCAGCAGCAGCAGCACCGCCCAGTGAACTAGCCTCAGAGACTCACCC
TACAGCCCAATCTGATGGACGCATTTTTCTCAACTGTGGTTCCCTCTTCTCAAATGACTCTAGAGTGTGTTAAATTGACATAAAACTAGCCAGCACA
CTGATGATTCTGGAGTGAAATCAAAAGGAGCTTGAAGATGAGGGTGGGGACAGAGGCAAGGGTATGTCTTAGACTTTACTCATTGAATTGCAAGCCG
AGAAGGATTAGAAGACATCAGCAGTATCAGTGGACAAGTGGCATTTGAATGCCATATGGAAGAAGGACAATAAGAAGTTCCTGCAGACCTGGTTCCA
TCCAGAAGCCTCTGCTCTGACTCCTCTCCCGGGACTCTTTAGGTCAAGGACTTTCTCCAGGGGTCTCAAGACTTTCTGTGTTCCTTTATACTAAGTT
TTCTTTCATCCTGTGAACAATCAAGTCATCAGTGAGGTGGTTGGGTGACATGGCCCATAAAGATGACTTCCAGGGACCGGGTTCCTGCTCACACTCT
GAAACCTTTGCTCCTGGGCAAAACTTCCCCTAAAGAACAATGACACACATTCTCAGAGAACAGAGAACAATAAGCCGTGTGTAACAACATCTTCCAG
AGAGAGAGAAGCGTGGATTTAGTCCTTTTGGGCCACGCTCAGCATCAGCAGGAGCTAGCTTGACATAAGGAGAAAAGCCCCTTAGCTTGGAGCTAAA
GGTCACATCACTTTTAAGTCTCAGTTTCTACAGCCACAGAACAGAAAGTTACCAAACACAGCAGCCTGAGGGTTTCATGTGAGAAGGTCATGGAGTC
CCAATCACTCACTCCTTAAACTTGACTCTGTCTGCAGCCCCCAGCTGGCCTTGAACTTGTGGCAGTGGAGAATGTGCTTGAAATACAGAAGCCAAAT
AGAAACCTGGAAATGTTACTTGTCACTGGTTGGAAGCCCTGGACCATGGTGGATGGACACTCGATTCTGAGTACCTACCCCCAGGACTCCCCAGAGT
AGATGTTTATAGTTGCTGGATGATGGTGGATGGACAGACAGACACGGACTGGCGCCTAAGAAAAGTCTAGCATGTCATCACGTACTCCAGCAGCCCG
AGAGCTTCATGTCAAAGACACAAAGCAAAGCCATCTCATGTGCCAACCACAACCTAGCCAATGGCCAGTTATCAAAGCCAGAAATCTGGCCTAGAGG
CCATAGCACTCTAAAATGAGGCCAAGTTAAACTATGTGGCGCTCTCTGCCCTCCAAACTGCCAACTTCAGCAGCCCTTTTGAAGCTGAGTCAAACTC
CTGACCATAGCGTTTCTTTTCACCACTGCCTTCCTCCCTCACCTGGCCCACTCCCTCAGCTCTCTAGGTGCCACTCTTCCCATAGGAGTCTTGGTTG
GACTCCTACTGTGCCTAAGCTATGCCAGGCTGTTCCCTAGCTAAGTCCTGCAGTGTTGTGATGCAATATTGTCCTCCATTATTTCCTAAATACCATG
AAATATTTAATATATAAAGCCATAAAAAGCCGGGCATGGTGGCTCACACCTTTAATCCCAGCACTTGGGAGGCAGAGGCAGGCAAATTTCTGAGTTC
AAGGCCAGCCTGGTCTACAGAGTGAGTTCCAGGACAGCCAGGGCTACACAGAGAAACCCTGTCTCGAAAAAACAACAACAACAATAACAACAACAAA
AAGCCGTAAAAAATAATGCAATGAAGACTTTAGTTCATCATAAATAAAACATCACCTTCCGAGCCCTTCCCAAAATTAAGGTGAGCCTGTTTGTGGG
TGCCATTTCTCTTCATATTGTTTGAGAGTGATTGTGTCTTGTCATTTGTCTCACTTTGTGTTCCTTGGTTTTTTGGCCACAGAGTCTCACTCTGTAA
AGGTGGCAGATCCAAGTGTGGCAATCTTCCTACCTCAGCTGCTGCTGCTGCTGCTGCTGCTGCTGCTGCTGCTGCTTCTTCTTCTTCTTCTTCTTCT
TCTTCTTCTTCTTCTTCTTCTTCTTCTTCTTCTTCTTCTTCTTCTTCTTCTTCTTCTTTTTCTTCTTCTTCTTCTTCATACGGTATTCTGCCTCCAT
GTATACCTGCAGGCCAGAAAAGGACACCAGATCTCATTAGAGATGGTTATGAGCCACCATGTGGTTGCTGGGAATTGAACTCAGGACCTCTTCAAGA
GCAGCCAGTGCTTTGAATGTCTGTGCTGTCTCTTCAGCCTTCCTCCTCCTCCTCCTCTTCCTCCTCTTAATAATTTACTTTTATTTTATGTACATTG
GTGTTCTGACTGTATATATGAGGGTGCCAGCTCCCTTGGATCAGGAGTCATAGACAGTTGTGAGCTGCCATGTGGGTGCTGGGAACTGAACCCAGGT
CCTTTGGAAAAACAGCCAGTGCGCTTAACTGCAGAGCCATCTGTCCAGCCCCTGCCTCAGCTTCTTAGTGCTAAGATTACAGGACTTCATCAGAACC
GTTATGACCCTACGATGGCTGGATTTGAGAATAAAGCTGTTGGGGGTTGGTTTTAATGCTTTGAATTAATCCAGCCCCCAAAATCGGGAAACTGCAT
GTCCAAACACTAAAGATCCTTGTCCCCAATTGGTTTTTGATCAATCAAAAAAGAGCCAATGGCCAATGGTTGGGCAGGTAGACTGAAACAAGACCTT
TAGATTTGCTCAGGCTGGGAACTGGGAGAGAGGAAGGAGAGAGCATTGTCGTGGTTTGGAGGGAAACAGATCAGAATTAGAGACCCAAAAGGAAAAG
CATCCGAAATGTAGGAGAGAAAGAATTCGGGCCCTGGAGGAACAGCTCAGAAGTGTCTTGGGCAGCAAAGACTGGGGTAGGGGCACCCAGAAGGAGC
CAGGGCAACAAAGATAAAATATAGAATTAGAGGGCATTAAGCCAGGAGTACCAAAGGGAAATGTGTTCTAGCCACAGGGAAGATTAGAACTGCCCAG
CCTTTGAGCTAGTCAAAGCATATTTAAAATTAACTGGTGTGTGTGTGTGTGTGTGTGTGTGTGTGTGTGTGTGTGTGCTTCATTCTTGAATCCAGAT
AGCTCTTGGGCAGGTGCATGGGTGTGACGAATCGGGAGCACAAAATGATTTAGCTAACTACTGCTACGTAGAGAATCTAAGGAAGTTATTTAAATTA
AATAGCACTATAGAGTAGGGTCCTGGTTCCATGGAATTGGTATGTGAGTGACATTACCAGAAATTGACCCTACTGGCCTTTGCCTTGGGACTTCCAG
CCTCTAGAACCATGAGGAAACATATTTCTGTTGTGTAAGACATCCAGTCCATGGTATTTTAAGAGTCCAAACAGACTACTCACTCCATGTAAGTTAA
GCAAGGCCAAGGATCATGTGTGCTTGTCTTTGGATCAATACTGTCCATCACTATACCTAACACATAAGTGCATGCCAAATATCTGTTTGTTGAATGA
ATGAATGAATGAAEGAATGAATGAATGAATGAATTAGATAATTACTAAAACAAAACATATAGGGTTCTGGGGTATGCAAGATGGTTTAATGGGTAAA
GGTGCTTGCTGACAAGCCTGACAACCTGAGTTTGATCCCAGGAGTAGACCTGGTGGGAAGAGGAGACTTACTCCTGCAAGATGTCCTCTGACCTCAA
CAAGCATGCTGTGCCACATGCCTGCACACAGCTCCTTTCTCCCTCTCCCTTCCCCCACCCCCATTCTCTCCTTCCTTCCCTCATGCCCAACATGAAA
GCTCTCGGCTACTGCTCCCATACCTTGTTTGCCTGCCTGCCTCCTGCCATGCCATCATCCACCATGATGGATGTGAACTCATCCTCTGAAACTGTAA
GCAAGCCTACAATTAAATGCTTTCTTATATAAGTTACCTTGGTCTTGCCATCTGTTCACAGGAATAAAAAACTAAATACAACAAATTATACATGCCC
CCCCCAAAAACCCTTGTAAATGAATATTTATAGCAACAATACTCATGATAACTAAATAGTAGAAACAATCCAAATAGCCAACTAATAGTAGATAAAA
CAAATGTGCTATATCCACACAATGGAATATTATTCAGCCACAGAAAGGAGTGGAGAGTTGAAACATACTCCATATAGATGAACCTAATTAAAAGAAG
ATAGATATGAAGGTAACATTGTGTCTATTTATGTGCATTAATCAACAGAAGCAAATCTACAGAAAGAAAGTAGGTCTGCGGCTCAGAGCAGTTATGT
GCACAGGTGGAAAGGACATGGGAATCATCGGAAATAATGGCTGAGGAGATCTGACTAAAAATGATTCAAAGTTTACCGTGGCGAATAAACGCCGTGA
CTATATAAACACCACAGAAATGTCTACTTTAAAAGAGGAAATGTGGGGACGGGAGAGATGGCGCAACAGTTAAGGACACTTGATGCTCTTGCTGAGG
ATCCAGGCTCAGTTCCCAGCACCCACACATCAGCTCACAAGGCCTGTAACTCCAGTTCCAGAGGATCTGACGCCGTCTTCTGACCTCCACGGGCTGT
AGGCACACAGGTGGTACACAGAAAGGCACTCAGGCAAATGCTCATACACATAAAATAAAAATCAATGAATCGTTTTCAAAGGTGAATTTAGATATGA
ACTATAGCTAACTAGAGCTGTTGTCTTTTAAAAGAAAGGAAGGAAACACAGATGAATTCCTTTCTTTCAACCTTCCATTACCTGTCAGTATAACCTC
ATGGCCAATTCTCTCCAGAAGCTAGAGGGAAAGTCGTCCGGGAAATGTAGTCCCTGTATGCAGAGCAGAGCAAGGGTATCAAGTGAGTGCCTAAAGG
GTTATGTTGTCATGGCAACTGTTTTAACTATTTATTTATCTTATGTATATGAGTACACTGTCACTCTCTTCAGACACACCAGAAGAGGGCATCGGGT
CCCATTACAGATGGTTGTGAGCCAACGCGTGGTTGCTGGGGATTGAACTCAGGACCTCTGGAGGAGCAGTCAGTGCTCTTACCCACTGAGCCATCTC
TCCAGCCCCCTGTTTTTAACTAGTTAATACAGAAGAAGGGCTAAGTCATTCTGAGCTCTGTGTCGGGTCCTGTCGCTCACTGAATTGTCTACAATCA
GTGGCTAGAGATAAAAAAATTTACAGGTGTCTCTCCTGGAAATGGAACATTTAGATATTTGCTCTAGAACTCAATGGCCACCCACAGTCCCCACATG
ATCTCTATGCTTGTTTTAAACCCACTCCTTGGACTGGGGAGGTGGCTTAGTAGCTAACAGAGAATTGCCCCACAAACACGAGGAACTGAGTCCAAAT
CCCAGAACCCTCATAAGCAGCTTGGCAGTAGCATGAGCATTCGTCATCCTAGCAGTCCCCCAAGGAGATGGGAAGCAGAGACAAGAGACCACGCACC
CAGAAGGCCAGCTCACCTTAGTATAGAATGGGCCACTCAATAAAGACGTGATACCTGCAACAGAATGGAAGTAGGCTGACACCTGACATTGTCTCTG
ATCTTCACACGTGCAATGGCATAAAATCCCCACATTCACGCATACGATCGCACACATACACGTGTGTACACACACCATACACACCACACAGGCACAA
CAATTAAAAATGCCAATAAATATATCAATCAGTTTCCCTGTGGGGGAAAATGGGTGTGGTCACAAAGAATAAAAAAAGTAAAATAAAAATAGGCCAG
GCATGGTGGCATATGTCTTTAATACCAGCACTCGGGAGGCAGATGCAGGTAGATCTCCATGGGTTCGAGGCTAGCTAGGTCCACAGAACAAGTTCTA
GGACAATCATGGCACTGACAAAAATAAAATAAAATAAAATAAAATAAAATAAAATAAATAAATAAATAAATAAATAAATAAATAAAATCTCTTCTTG
GGGAACCAGTTTGGATAATCACTTAGACTTGATGTCCTCCTCGGTTAGTAGAGAAAGCCGAGGGGCTCCAGGCACCCTCATATCTAGCATCTAGGTC
CTGTGATGTTTAGCTTAAGTACTAAAAATAAACACTCAAACTTTCACTTCGTGGTTGTGTCACTGAAGCCTTGCTGGCCGGACCGTTCAACAGACCT
CACCCTGGCTGCGCAGGTGTATGTCGGGGCTGCCTGGCGGCTGCTGGGACAATAGCCACTGATCAAGTTCATACTGAAAGGGAGACTTTTATTCAAA
ACCAAGAGAAGGATGCAAGTACACCTCTGACTCCCGAGCCTCAGCCTATCTCACCCCACAGCAGCCACCCAGGCGTGCTTGGCACTCACAGATGTAA
GCAGCTCCTCCCTGAGTCTCTACAAGGCACTACTACCAGGGGCTTTCTGAAGGCAAACGACTCAGTGATTCAGGATCCTATGACAGCCGAATGTGGT
TATCACATACCTTGTGGCCCAGGAAGACTCTGAGGGGTGGGACAGGAGTGGGAGGGGCTCTGGAACAACTTAAAAGGGGCTCTTACCACTTCCTCCC
TGCCAGAGAGGGCTAGCTGGAGAGAGACAAGAACCAAAAGCACAGCCTTCCTGTGTGATTTCTACAGCCCCCAGAGCACCATGGACCCAGGTAAGTA
AGCTTTCTCTTAAGAGAAGTCAAAACTTCTGACACAAAGCCAGACTCTAGGGCAATQAACATGCATGGGGGATAGGGGTCATQGQGGGGGGGGGGAG
TTTCCCAGAGCAATCAGGGCAGTTGGTGTGAACTGGTGGCTGCTTTCAGAGAATGCCATCCCGTTGGATGAGGCATTGGAGAGAGGATGTACCTGCT
CTTTCAGACCCAGGGGAAGGAACAGCTGTGGATGCCAAGACAGACACAGGCATAAAGTTGGGTCCTCTCAAGCAGCAGGCAATGAACAGCCTGGTAG
ACTGACCTTGATGCCTGGATAGAACACGTTCTCAGCTGGTAGGGATGGGTGATGTTCTCTGGCACCTCTGGGGACTAAGAAAATTCTCTTTAGAGAG
TTCAGGACACCCTGCTCCCACTCTGGGTCCCTCCTTTCTGTCCTGTTAAACCAGGATTTGAATCTCCTTGCTCACGAGAAGAGTGAAACTGATGGAA
ACTGTGCTGTGCATAGTGCAATACAAATACTAGCTGCCACCTCTGCCTCTTGTTGTCTCTGCCTGTCATAGTCTGTCTGTCTCTGTCTGTCTGTCCT
CCCTCCCTTCCTCCCTCCTACACATCCCAGGTTTTGTGCCTCTTGCAGGTTAAAGGGTGAATGACTAACATAAAATTGAGTGGCCCAGGGGGCACTC
CACGTAGAGAAACTGTAGCCAGCACAGCACAGCCCCTAAAACAAAGCCCACACACCACAGCCCTCTCTATAACAAGCTATTCTACCAGGCAAGCCTC
AGTGTCTACTATAGCAAAGTGCCAAGGGGAGTCCAGCTCCAGACACCCCAGTTACACATCTTAAATCTCGGCTGCAGAGTCAGACTCGGCTGGGGCT
GGAGGGGCCAAGTTCCTAGAGTTCCGCTCACCAGCCTCAAAGCCACAAGGGGTTGACTTTAAAAAGTGAAGGTTCCCTGGTGCTGTCCTCACACCCC
CTCCAAGCCACACCACAGCCAGCAGCATCTTTATCTGTCTCTCCTGCCCAGCGTGCATTGCACCCTAGATCTAGATCCAGCTGTGGAAGGTATATAG
TGGGGTCCACCTCTGCTGGCTATGAACTCATGGGAAGCAGGATTTTGTTCTTACACCTCTGCCTCTCAGATGTTTTGGGTGTAAGTAAAAAGTGTTG
ATGGGAGGGAGGGAGAGAGGGATGGAGGAATGGATGGATGGATGGAAGGATGGATGGATGGATGGATGGATGGATGTGTGGATGCATGGATGGATAG
AATCAGCCTCTCTAAGTATATTTTAGAAAGCAAATATCTATTCCAGCTTCTCCTCCCACCTGCCTGCTTTCTAAGCCTACACCTCAGGTATCTACCT
CCACCAGCCATAGTCTCAGAGAACCCACACCATCCACCAAAGATCTTCTCCACCTTCCTAAAATGTGGCTCCCAGAATAGGGAAAGGTTCTAAATTT
ACAGCATTTGCCAATTTCCATGGTGTAAATTTTACCATCCCCAATTGACCTTAGTCGGTCCCATTAAAGGTCTAAATGTTTTGACACAAAAGGAGAC
AGACACCTGCCTTCCTTGGCACTTGGGACACTATAGAGTCTCTCCTGAACTAGGTTTGGGGATGTAAATGCATGGCTTTTGATACAGGGGGTGGGGC
AAACTCTGGTCCTGGAAGAAGAGATAGTCTTGCTTTTTTACTCAACTTAAGAAGCACCCTAATAGCTAACTCTGAAATGCTGGTGTGAGGGGAAACT
GCTTAAGAAGTTTTGCTCAAAACAGGCTCCGGCGCGTTGGAAGGGCGGGGTGGGCCACAAACTATCTGTTTTTCACTCTTTGTCTACCTAGTAGGGA
CAGGAGGTGGGTGAGAAGGCCTAGGTTTCTGTTATACAAAATGAGAAACTACAAACCAGGGCTGAGCTGGCCTCTTTCATGATTTGAATGAGCAGCC
CTGATATACCCTTCCTAACTTCAAGACATGGGACCACAGATCTCAGCACAACATCTCAAAATCAGAGAAGGGGAGGGGGTGACAAATGAACCCATAA
GAGACTTGTGTCTCACAGCTCAGCTGTAGATGGCAGTAGAAGAGAATTCAGGGTCATTCTCTTGTGGCCCCTCCAGAACCTGGCTGTGGAGAACACT
GGAGCCCAGAGTAGCTAGGAAAAAACGCTCCTCTGTGTGGTATTGTCTTCCAGTCATGTGTCACTCATTGAGAACTTCTTGAATACAGAGTCTGGTG
GGGAACGTAACATGTATGTCAACACGCAACACCAACAGTGAGAGACACAGCACTCCTGAGGGGGCATAAGGACAAGATTCAAAAGCAAAAAGCAAGG
CTGTGAACACGTGTCCAGAAAGCATGGCAATTGTATTTTGAAGGGTGTGTAGAAGTTGTCAAACCTAATAAAGAAGGGCCAGGTTGTAGCCAGATCT
TCAGACTCTACAGCCTTAAGCCAACAAAGTAAATGAGGCATCCGAGTGTCAGGAACGTGACTGGCACACAAGCCTGAGGAACCTGAGCTTGTAGCCT
CAGAGTTTAAAAGCTGGCTGTAGCAAGGTACACACGTAATACCTAAGCCTGCTTAGGGAGGCAGGAATAATAAGACTTGCTGGCCAACCAGTCTTCA
GAATTGGCAAACTCCAGGTTCCATGAGAGACACTGATGTCAGCCTCTGACCTCTGTATGTGTGCACACCCATACATATATGAGCACACACCTTGAGC
TCATAGTCAGTCAGCTCTGGTGAAGGCAATAGAGCAACAAGGAAGGGGGAATGGTGTCAGCTGATGGGAGTCAGGGAAATAGATGTGCCAAGAAACA
AGCTAGACTGGGGTGGCCCTTACAGCGCTGTGTGACAGCACCAGGGAATCCATACACCACCCTAAAAAGAATGGGGGCCAGCAAAGGTCCCCCACCC
TTTTCCAAATACATCGGCAGATAGATGAAATGTG~GTTTTGGAAGGAGTAGGGGACAACAGATCCTTGTATCAGGACAGGGGAGGGACCAACATCAA
AAGATGTGGAAGATCGAGATGTGGAAGCAACAGACACGAGTGGCCAGAAGCAGTAGAGAGAGCAAGAGTGGCCTTGATCTGTGGAGGACCCTCCACA
CCCAAGGCTCATCAGTAATGTCACATGACTCAGCCATTAGAACCCAGGCTAGATGTTCCCCTTTCCCATCTCTGATCTATTGTTTAATAGCTATGTG
AATCCAGAGGAACGACATAACATCTCTGGGCCTGTTTCCIAGAATACAAAAGGAACTTACAAGATGTTATGAGGGGGGAGAGATGTAGGGAGAGGAG
CTAGGAGTGAGGTGGCCATGGCCAGGGGCTGGGTGTCCTTAGGGAAGCCTTGTGCTGACAGTTCTTCCGTTGGGGCTGATTGTCACGTGGGGTCTCA
CAGGGTATCCATTGAGGACGGGGATGGCAGCGCCTGTCTCACGTGGACAGCGCACTTCACTGAAAGCTCAGTGGGTGCCAATTGCATCACTGAGTGA
CAGCGTTGGCCCTAATAACAAGATCCCACCAGCTCTCACATCCCTGCCTCCCATCTTGCGGGCTGGGTCCCTGCTTTCAGCCTCTGCCCTTTCTCAT
GTCCCCTACAAGCGGCTGAGAAGGTCCAGAGAGAAAATCATCACCTQAGCTGTCTCTCGGGCCAGATGATAAGCAGCCATGGTTTCCTGGGCAGCCT
GGAGGGACTGGGGGAATGTACAGATGGGAGCTTTAGGACCTGAGCAGGAGGCGACTGGCTTCCCAGGCTGGGAGCTGGGGQCGGGGGAGGGAATTGA
GTGTCAGGCTGAGGCAGGGCAGGGCAGGGGAGTTGGGATGGTGTCGGGCTAGAGTGCTAGGGAGTTTTCCTTTCACCTTAATCATTCTGCAAACCCA
AGAACTCAGGAAACACCGAACACCTCAATCAACAGCTCCCCCACACTGGCATTCCCAACTGCCCGTAGCTCAAGAGCAGTTGCTTTCTGACTATTGA
GTCATCTGACTCACCAACACTGACCAGGTCCCTCTGTCACCACACGTTCAAATTCCCCCAAATTCTCCTGTTAACTGCCTTGAACATGGCCTGTCCT
GGATCTACAGGACCCCGACTCACCCACCCAACCCCCACCCCCAACCTCATATCCAGTATCCCTGGAATGAGACATCAATTTTGTTCACACACCCCAG
AGCCCCTCCGGGTTTGTGGCTACAAAAGAACCGGGACAGAACCCAGAGTAAAACAGAAACAGTTCTTGTTCCTTAGGCGTTTCTCCACGTCCCTGAC
AGTCAGATTGGGCTTTCTCTACAGTAACCGAAAGCCGTTGATGGGTTGAAAGAAACCTCGAACGCTGAACCCCGAAATGAGTCAGGGAAGGGCGGGC
TGTGCATCAGACCCTGAAAATCCTATAGTTGAGAATGGGCATGTGACCTCAAGCTCGTGGGGTGGGGGTGGGGTGCCAGGGGTAATTCAGTTGGGGG
GGGGAATGTGTGAGAGAGATGAGCTGTGAAGCCACGTGGTCATGTTCCCCTTCCTCTCTCCAAACGTTCTAGTACAAGCTGATGATAAGGGGACAGT
GGCTGTCTGTCCCCAGAAAAAGATGGAGCTGGTGAGGCATGGGCCCCACGCTAAGCCTAAGCGTCTCTCTATCTCTACAGCTAGGATTTCGACTTAT
GGTTAGGGATTTAGGAGGGAGGCAAGATAGGAATAGAGGGCAGAGTTAGCTTCAGTAAGACGTGCTTACATAGCATGGAGGAGAATGTAGAGGGGAA
AAACGTTTGTCCGGGTCACCCTGATGCCCTTCCTGCCTGTACTGAGTGACCAAGCGTAGCCTCCCCAGCCTGGAGTACATGTCAGGTACCCTGGGTG
TCATGTAGCCTAGTCTCCAGCAGGTGGTGCTATGGCCAAGGACAAACAACATCTGCGTTTGAACTGTTTCCCTGGCCTCAAGAGGTCCCCCAGACTC
AGATTGGGGAGCAAAGTCCTACTCTGGACTGCAAGACCCATAGGGTCTGACTCTTCCTGTAGATCTGAAGAACTTGGATTCAAGGGCTGGAGAGGGG
TGCCAATAGCAAAAGAAGGCGGACAAGGGTCCCCAGGGGTGAGACGGGAGTATGTGGGGTAGAGGTCTGGGGTGGGGGGCATAAGATGTCTGCAAAA
ATTACTTTTCCAGTTGTCCTCTTGCTTAGCTGTCAGGAGAAAATACAGCCCAGGCTGGCTCAAGGCTCAGCTCTGAGCAGAATGAGGAGTGGTCGCT
GTCATATNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNCTGGGCAGCAGGCCAGGTGTTCAAACACACAATCTGGTTCTGATGTTCTCTTTCAGCA
AGCCCGAGGTGCCTGGAGCCTGGGATAAAAGTTCCCAATGGTGCCCCTGCCCCACCCCCGAATGTCTCCTTAATCACCGAGCAATTTTTACCGGGGC
GGTAACATCATCTCCTTTGTCTCCTCTAGGGAAACCCAGGAAAAACGTGCTGGTGGTGGCTCTCCTTGTCATTTTCCAGGTGAGGCTTCCTGCCAGG
GCAAGAGCTCTCTTCCCTCCTCCACACCCCCTCCCCAGCCAGGAGTGCTGGAACACAAACAGACCCCGCTGCAGGAACGAAAGGTCTAGACACAACC
CACCTGCCGTTCACGGCCCCTTGCCCCCCCCCCGCCTCCCCCCCACGAAGGGGGCAGGCATTCTCTGGACTTCCATTGTCCCCCTTTAAAATAAAGC
CCAGGGAGACGTAACCTACCCCCACCCTCGTTCTTCCCAGCTCCCACCTCCTCACTCAGGAGACAGAAACAGTGTCTGAGATCTCTTAGACTTGTGC
AACACAGGGCTTAGAGTGGGCCTGGTTTTGCACATAGGTGCACACATCATCTGTGAACTGAGAAAAGGACCCTCGGTCGGGCGCTCTGGTTACTCCC
CAGTCACCCAGAGTTGCTTCCTCTTGCCTTTCAGTCCAAGGGTCTCCTTGTAATCTCATTCTGTTTTCCTTCATAAAATAGAAAGACCTTGGCTTGG
CTTGCCTGTGGGGAGCAGGAGGGAGAATGAGTGGAGCCCGGGACCCGGGTGGAGAGGACGGTGAAGGGAACACATAGTGGGTCTGAATTCTGCAGAG
CCAAGAGCCCACTCCGTTCTCTATCCCAGCAATGGAGGCAGAACGTGTGTTCCCCCAACCACCAGAGTCAGGAGAAACTCTGAGAGTCAAGTTTCCT
CAATTGGGGTAGGACTCTCTCAAGGTGGGGAATGGCCACGGCCAGCCTGGAGAAGCAATGCTTCAGGATTTGACTTTCTCCCAGGTCCGGAGGCCAG
AGGGGGAGCTTTCCAGGAGCTCAGGCCAGGCACAGGCCCCAGGCTGGGCACTGTTCCCCAAACCCCTCTCAGAGGCCCCCTGGAGCTTGGGTTGCAG
CCCCTTAGCCTGGCCCTTTCCCATCACCACTCCCGCCCCTCCTGTACTGCTGGCTGGAAGGCCAGAGAAGGGTAAAGACATCTGTGGCTGGTTTTTT
GTCTTTCACTTGGGAGGGAAGGGGGGGGGGGGTTCAGACAAGGCCGGATGTGGTGGGGCTGTGGGCTGCAGAGGCGGCAAGCTTGGGGGAAGGGGTG
TTGAACAGGAGGGAGAAGAGAAGACTGGGAGGGAGGTGGTGTGGGAGCACACTTCATAGAAGCTGGGAGTTCTGACCTCCAAAGGGTCTAGAAGCCC
TTTAAGCCTCTGGGGCCGCAGACCTAAGGCCACAGACCCATTCGTGAAGAGCCAAGCGCAGCTAACCTCTCCCAGGGCCCCCAGGCTGCAGTCAAAC
CTGGACCAGGGAAACAAGGTTGCTATGGACTCTTCTTCTCAAGTTAGCCATGCCCCAAGAAAGGGCTCCTGAACACCACTTTGTCCTTATCCTGTCG
GTAGGCTCACAATGGTGGTGCGATCTGTGGATCACCACTGAATGACTGAAAATCTCCTACCCAGAGGAGGAACCCTAAACTTTCCAAAGTGGGCCTT
CAAACCACCCCACCCCCCTCCCATCTGTGGTGGTCCATCCATCACACACAGGCAGACGGCAGGCACCCCCTCCATTGATAAAAATCACAGCAGCTGC
TGAACCGTCCCCCACACGCACCCTGCAGCCCTGCTAAAGGGCTAAAAAAAGCCCAGAACAGCTGAGCCCTGCTGATTGGGATCTGCCGGAAGAGAGG
CAAAGAGGTGTGGGAAGAGGGTGGGAACACAGGGAGAGTGGGGGGGAAGAGAGCCGCTTCCTTCCAGGGGCACAAGCCGCCATAGGAAGGCAGTCAC
ATTTACCCAAGCACTACCCCACAGGCCCTACGGTACATTTGTCTACGCGCTGAGTGTAGTGGGTACCAGGGAGCTGTGAAAATCCCCATGTCTCTTA
TCAGCAGTCCCTCGACTGAAGTTTATTCCTACAAAGGAGCTTTGATCTTCTCTTCCTGCTTCGGACCTTCCTCAACCTTGAACCTAACATAGGGGGT
TGTTTTTTGTTTTTCCCTCGAAAGCTGAGGCATTTTGTAGCCCCCCACCCCCAAAAAGAAAACTCTCCTGGGACCCAAAAGAGGCTCCAGAAGAAAG
ACACACTTTCCCCCCACATCACAGGGTCCCAAACCCCACAACGGTCCAGCACAGGAGTTGCGATTGCCTCATATCTCCCAGACACAGAGAAGATGTG
CGATTTCCAGCTCAGGGCTCAGCAGACCCCAAAGCCCCTAGACTATCCCCAATCTCAGCCAGTGAGGATGAGACTGGATGTCCCAGGAGTTGACAAA
CATCAGGCCTTTGCTACTGATGAGTAGAAATGTAAACTTTCTGGTCCTGCCTGAGTCTGCAGGGCCAGACGTTTACATCTTAACTAGATCCTCCCTG
AGTAATTCATACATGTGCTAACATCGTTACTTAGATTTATTTTTTGTTTTGGTTTGGTTTTTGGCTTTTTAAGATTTTTTTTATTCATTGAACGTAA
ATGCATACACTGTAGCTATCTTTTAGACACATGAGAAGAAGGTATCAGATCCCATTACAGATGGTTGTGAGCCACCGTGGTGGTTGCTGGAATTGAA
CCCAGGACTTTTGGAAGAGCAGTCCGTATTCTTAACCCTATTTAGGATTATTTTATTTTATGGGTATAAACATTTTGCCTGCGTGCATAGAAGTACA
CCACATGCACAGAGGAGGTCAGAGTTGGGCATCAGAGCCCATGGAACTAGAGTTACAGAGAGCCACGACGCACCGTGTGGGTGCTGGGAACCGAACC
GGCCTTCTCTACCAGAGCTACACGCACTCTGCTCCTTAACCCCTGGCCAGTTCTTCAGCCCCACATGCTGAGGCTTAAGAGGTGTGGATTTCTAGTC
AAAGACTCACTTGGGGTTTGAGGGGGAAACTATAGTTTCCTGGGCTCCTCCGTCTAAGATGCTGACCCAAAGGGTCTAAGGTCTGAGAGTCTGCAAG
AGAACAGAAGCCCCGGGCAATGTCCTGACTGTGAGATCCGGACTGTGAGCTCATCAGGTGCTTCCTTCCCCCTTAGGTGTGCTTCTGCCAAGATGAG
GTCACCGATGACTACATCGGCGAGAATACCACGGTGGACTACACCCTGTACGAGTCGGTGTGCTTCAAGAAGGATGTGCGGAACTTTAAGGCCTGGT
TCCTGCCTCTCATGTATTCTGTCATCTGCTTCGTGGGCCTGCTCGGCAACGGGCTGGTGATACTGACGTACATCTATTTCAAGAGGCTCAAGACCAT
GACGGATACCTACCTGCTCAACCTGGCCGTGGCAGACATCCTTTTCCTCCTGATTCTTCCCTTCTGGGCCTACAGCGAAGCCAAGTCCTGGATCTTT
GGCGTCTACCTGTGTAAGGGCATCTTTGGCATCTATAAGTTAAGCTTCTTCAGCGGGATGCTGCTGCTCCTATGCATCAGCATTGACCGCTACGTAG
ACCATCCAAGTGGCCCAGATGGTTTTTGGGTTCCTAGTGCCTATGCTGGCTATGAGTTTCTGCTACCTCATTATTTTCCGTACCTTGCTCCAGGCAC
GCAACTTTGAGCGGAACAAGGCCATCAAGGTGATCATTGCCGTGGTGGTAGTCTTCATAGTCTTCCAGCTGCCCTACAATGGGGGTGGTCCTGGCTC
AGACGGTGGCCAACTTCAACATCACCAATAGCAGCTGCGAAACCAGCAAGCAGCTCAACATTGCCTATGACGTCACCTACAGCCTGGCCTCCGTCCG
CTGCTGCGTCAACCCTTTCTTGTATGCCTTCATCGGCGTCAAGTTCCGCAGCGACCTCTTCAAGCTCTTCAAGGACTTGGGGCTGTCTCAGCCAGGA
ACGGCTCCGGCACTGGTCTTCCTGCCGGCATGTACGGAACGCGTCGGTGAGCATGGAGGCGGAGACCACCACAACCTTCTCCCCGTAGGGGGCTCCC
CTGCCCGGACTACAAGGACCTCTCCCAGGAGCCTTAATGTGGTGCACACATGCACAGACTCTCCATCCACCGAATTGCTGCTGAGGGAAGAGCAATT
CTGGCCAGTCAGGTTGACATGAaGACCTAAGAAACTGCTTAACCCCATCCCACTTATAACTACCTCAACCAAAGCTGTAAAAGATATGGCTGAGAAG
TTAACACTCAAGCCAAGACAGCTATCCCCAAAACGACAGCCAAAAGTGAAAGTGAGAGGCTGCCACACTTTCCGGAGTGAGGGATGTGGGGCCAGTG
AACACCCTGGTTGAGTAGTCTTCGGAGGCCTCTGAATGAACCTGCTTCTAGCTTAGAGAGATGTCCCGGAGATTCAAGGACAGAGCTTATCTCCACA
CTTAGCAAGCAAGCAAGAGATGACAGTCTCTCTAAATGCTCCCACAGAGCACCCCTGCCCCTCCCTTCTGCCTCTCCACCGCCTTTCCTGAGGTCCA
GGCCACACCATCAGCCTGAAGGCAGTCCCAGCTGGGGCTCTGGATGGCAATGACAAGTAGTTGGGTCTCTATGATGGGAATAAAAAGGTAGGGGAAA
GGTGACAGAAAGGAGAGAAGGTGACCCTGCTGGCTGACAGAGGCCAGCAAGCTACTTCTTTGTTCTCTGrCAGCCACTGATACCTTTCCTCATGTTC
TGCTTTTGATTCATATATCTTTTATGAACAAACAAATAAAAAAAAATTTTCCCTCGAGGAAACAACTTGGAAAGAAGGGAGGAAATTCCTTGGTTAA
ATGGCGAGTGAGTGAGTTGTCCCCCCATTCCACCTGAGAGTCCTGCGCCCCACACCCCTCCGCCCCAGCCTTCCTTTCTCAGCACTCAGAACCATGA
GGTCACAGAGCCTCTCCGGAGATGCAAACCCAGGAGGCTGCAAGAGGTGGAAAAAGAGCGAAGACAGGATGTCTGTCTTGCACATACACCTGCAGAG
CCCGTGGGATGGGACCTAGGCCCTTACAGGCAGCAGTATACATAGGAGAGCACTCTGAGTATGGAAAGACGAGGTTGTCCCAGGAACTCCTGTGTCT
GGGAACTCAGAGGAACCCCGGGTTGGGGGGAAATGGACACCTGCATCCTGTTGTGTTTGCTGTAACAGaATAGCCCGGAAACAGCCCTAACTCTTGT
CAGGCCCCTTCAGACTTGCGTACATTCCTCTAAGAACTCCAGTTTGGTGGGAAGCTCAGACAGAGGTCATGACATTCTAGAGTAAGGGAACTAGGGA
CTAGTCAGATGCCACAGGCTGGATGGGACTCTGCAGGGTGTTTGAGAGCTAGGACCACTCGGTCCCCTCCTTTCTACCCACTAGTAACAGAAGTTAC
TGTTACCAGCCTGACCCCTAAGTGCCTCTGTAGTGCGAGTCTCAGTCCATCCCCGTTTCCTGTAACAAAACACTGCGGTCTGCGGAACTTATACACA
ACAGGAATCTATCTCTCACCATTCTAGAAGTTAGACAACTTCCACTCTAGTATCTCCCAAGTCCATTTATAAGGCCTTAAATCCATTCATCAGGGTG
GAGCCTTCACGGCCAGCCAGTTATCTCTCAACCACCCCACCCTCCAATACCATCATCCCCTAGGGTTCAAATTCAAACACAGGCCTATTGGAGGGAC
ACACACATTCAAAAGACAGCGGTGTGGAAAGCAGAGGGAGAGGGCCAGACTCTGTCGCCCCTCACTCCATCCCCACAGGCCAAGTGTCCTTGTCATT
GTGTCCCAGTGTCTAACCAAAGCCCTAATTTCCCCAAGCCCGAGAGCTTGGTCCAAACTATGATACCTGGGCAAATGTCCAATGGCTTAGCACCCGA
ATAGCTCTGAGCCTGGCGGTAGGATGAGGGTGGGGGGTGGGAGAAAAAAGGTGAGACATTCACACAAAAACATAGTTCCAATCCCCCCCCCCCCAAG
AAAAAAGAAAAATGGATTCCTGTTCCATAACAGTGCTCATTCTCTAGCTTGTAACCACAAAACACCCATTACCCTCCTCCACAGGATGCATGGGGCT
AGAAAGACTTTAACACAATTTGTCAGGCGGCCTCCCGGTCTGGAATCAGCATCGTGGGAGGAACGAGGCTGGCTCAAGTGCAGGCAGTGGCTGGTGG
CGGGTGGCGGGTGGCGGGCAGCTTTCGGGATGCTGGTACTGAATATTAAAAAGGTTCAGTTGCGACTGTGGGACCACAAGCACGCTGCAAGAAGGCG
ATGCCACTGGCCACGGTCATCCCCAAGGGGAACCTGCCAGAGTGCACGTGGCTTTGTAGATCCTAATGAAGTTACAGATGGGAAAACAGCCCTCGAT
TCCTACCAGGGAGGAAATAATGGAGGGATCACGGAAGGGGAAGCTGGGCCAACCACACAAGCATGCCACCCTGGCCTCAAATACATTGCGCTAGTAT
AACTGGACATCTCCAAATATTTACAGAAACAACAGAACCCCCAGCAATGCTAATTCCCCAAACTGCTGTGTCTCAGAAGTTCTATCCTCAGCTTCAC
GCTGGCCTGCTGCCTAAGACCTGGCCTGATGCACTCTTGTGCCTACCAAGGGTGATCCCCAGGATACAAGAAAGCACCCTGCCTGAAGCCCACCCAG
GTCTTCTTTCTCATTCTGCCCTTCTCGTCTGCTCCATACAACAGCCCCATGCCTCTAAATCTGTGTCCCTTGCCACGCCATCATTCTGCAATTGCCC
CAAGCTCCTTCCAGTCCACTAACAGTCCCTTGTCCACGCCCCCCACGCCCAGTCCCANNNNNNNNNNNNNNNNNNNNCACACACACATCAGCCCTAA
CTCAGATCCCCCCTGGCTGTGACCCTAGGCCAAATGTAAGACATTCCCAGGTCATGAAACTGCCCTTTCTGCTAGAAGTGCCGAACCTTTAATAGGT
TCCTGGAGACCTCAGGAAGCAGGGGCTGACCCCTGTCGTTCTCTAGACAGAGCAAGGAGCTGGCTGGTCCAAAGAGACACAGCAATGAGGGGAGGTT
GCTTAAGCTAGCTGTGGAGCCTGAGGGTCTGGCAGAGTTGCCAAAAGGGGGGATGGAGCCTGGCCTACTCTGAATCCCAACCCTGCCGCTCCCCAGC
TGTGTGACCTTCAGCTGTTGCTCAACTGCTCTGAATCTCAGATTTCTCAATTGCAAAACAAGGGAGATTTGGGTAAACACTTAGCACAGAGCCAGAA
TGTTGAGCGAGGCAGCAGCAGGGGAGCTCCCACTCCACAGCCAGATGGCCTGGTTCATGTCTCCGCTCCCTTACGGTGCGGTGCCTCGGTTTCCAAA
CCTGCATAATTGGAGTTATTAGTCGTGCCTGCCTCCCAGGATTGAGGTAAGGAAGCAATTTTTTCTACAAGCAAAATATTTAGAAATACCCGGGCAA
GTGATATCATCTTTTGGGAGATAGTTGAGAAATTATAGATTTAAAACATTGCAGGGATGGAGAGGTGGGTCAATGGTTAGAGCACTGGCTGCTCTTC
CAGAGGACCCAGGTTCAGTTCCCAGAATCCAQATGGCAGCTATTAACGGTTTGCAAATCTAGTGTCAGATAATGAGGTACAGTGCCATGTATGTGTG
GGGTACATGCCTACATGCAGGCAAGACACTCATACATATAAAATGAAAATAAATCTTTTGTTTTGTTCTGTTTTCATTTTTTGGAAACAGTGTTTCT
CTGTAGAGTCCTAGCAGTCCTGGAATTGACTCTCTAGAAGAGAATGGCCTCTAACTCAGAGATCCATCTGCCTCTGTCTCCCAAACCCTGGGGTTAA
AGGTGTAGGACACCACCCACCGGCATAAATAAATATTAAAAAATAAAATAAAAATGTTTCTTTAAAGAGACAGAGAGAGAAAAGGCACAAGAGATGG
CTCAGTGGGTAGTGTTTTGTTGTACATGAGGCCCTGAGTCCAGATTCATAACCCACACTCACAGGCCAGGGTTGGTGTTGCGGACGATGGGAAACAG
AGATGCTAGAGCCTCTGGCCAACCAAGTCTAACAGAAAGTAGAAGCTTCAGGCTTCACAAAGAGACCCCCATCTCAATAGATAAGCAATAAGTAAAT
GATGGAGAACAGAGGCAGACACCCCAGTGTCTCTCTGCCCTCCACCTGCACACACACGAAGGAGAGAAACTCTTGCTAAGTTCAAGGTCACCACCTT
CAGTTCCTCAGCCCTATGTCTGTTCACACGCAATCCACAACAAACTGGAGGACACCTTAGATGAGAGTCTGGTCTCCCCACAGGGTGGCCAGGAGAG
ACATTTGAGGCACAGGAGAAGGAGATCACCTGAGGTCTCTAGGGGCCTCTCTGATACTCAGAATCCTAACGCCCTTTAGGTCCACCCTCAATTGCGG
TCTGCCAGCATCCATCCTTAATACGAACQTCTCTTGCCTGCAGACAGCTCTTGCCTTGATAATGAGTCTTCTTTGAGAACGAGGTACTCACTTGCGG
GTTTTTTGAGTCAAGGACAGAGTAGGTAAGGAAGAAACAAGCCTATGAGATGGTGGGAAAAAAGGACAGAGTCGTAGAGGAGATTCTGAATTCGGAG
GAAGGAGCAGGGATGGGGGAGCTGTATATTGGAGACGAGACATGGAGAGTATATCCAGTGGGCAGATGAATCCGTGTGTGTGAGTGAGTGAGTGTGT
GTGTGTGTGTGTGTGTGTGTGTGTGTGTGTGTGTGTGTGTGCCTCCCTCAAGTCTCTAGTCTCACAGGAAGAAATGATTCCATGGGTGTTTGTTCTT
TTCAAGGGAAAGGTTAGCCGGGGTGTGGTGGCGCACGCCTTTAATCCCAGCACTTGGGAGGCAGAGGCAGGCGGATTTCTGAGTTCGAGGCCAGCCT
GGTCTACAGAGTGAGTTCCAGGACAGCCAGGGCTACACAGAGAAACCATGTTTCAGGGAGGGGGAAGGAGGAAGAAGGTCAAGTTTCCCTACACTGA
GTCCCTGAGGACTCTCACTCTCACCCCAACAGGAACCCAGCTTTTCCACACACCAGGCATCCCAGCCAGGGACACAGATGAAGGCACTGTCAACAGA
AGCACCAGCTAGAACTTCTGTGATCCTGAGGTCACATGGCCCAGAGGTTCCTCCCTATTCTGATTTGTATTTGTGACCCGATCATTTCCTTGCAGAT
GTTAGGTTTACCCCTTCCCTTGTGCACTTTGAAAATGGCTACTGCCAGCGGGGGGGCGTGGTGCCGCATGCCTTTAATCCCAGCATCAGAAGGCAGA
GGCCAGCTGATCTCTGTGATTTTGAGGCCAGCCTGGCGTACATAGTAAATTCCAAGATGCCCAGGAGAAAAAGGAAGGCAGAAGCAAATGGCTATCG
CTCTATTTGTCTACTGCCCACTGATCCCACAAAGTTCCCTGACCAGCGTGTGTGTGGCACTGTGTCACACACCACAGTGACCCACGAGTTCTCATCT
CCCAGTGTGACCTACGGGTAGTCTGCCCTGAAGTGGATACCCAAGTCCTCTATCACGAGGGCAGGAGAGGGACTTTCTCAATCCATGTCACAGCTTG
CCTAGGACAGTGTACGTTATGTTTGCTCTAAACAAATATCATTGTGAAGCTGAACAGATGTTGCAGATAAAATGCCCTAAACCCTTTCCCTACGTTT
CAAAGAAGCTGGGAACTCAGGCGTGCTAGTCTTGGATGGTTTCTGCATCTGTGGTCCAGTAACTGGCACGCGCAATCTCTCTTGCAAAGGCAGAGGT
GAATTCTTCTGGCCCCGGGGGTGTTTTCTGCAGCCTGGCTAGCTCCAGACCTGTAATTAAGTCTAGTGGTATGCTGGCAAGGATGTGGAGAAAGAGG
AACACTCCTCAATTGCTGATGGGATTGCAAGCTTGTACAAGCACTCTGGAAGTCAGTCTGGCGGTTCCTCAGAAAATTGGACATCGTATTACCGGAA
CACTGGTAAGTGGATATTAGCCCAGAAGCTTAGAATACCCGAGATACAATTTGCAAAACACAAGAAAATCAAGAAGAAGGAAGACCAACGTGTGGAT
ACTTCATTCCTCCTTAGAATAGGGAACAAAATACCCATGGAAGGAGTTACAGAGACAAAGTTTGGAGCTAAGACGAAAAGGTGTACCATCCAGAGAG
TACCCCACCCGGGGATCCATCCCATAATCAGCCACCAAACCCAGACACTATTGCATATTGCCAGAAAGGACCCTGATATAGCTGTCTCTTGTGAGGC
TAGGTCAGGGCCTGGCAAACACAGAAGTGGATGCTCACAGTCATCTATTGAATGGAACACAGGGCCCCCAATGGAGGAGCTAGAGAAAGTATCCAAG
GAGCTGAAGGGGTCTGCAACCCTAThGGTGGAACAACAATATGAACTAACCAGTACCCCCCAGAGCTCGTGTCTCTAGCTGCATATGTAGCAGAAGA
TGGCCTAGTCGGCCATCATTGGGAAGAGAGGCCCCTTGGTCTTTGCAAACCTTGGTACAGGGGAATGCCAGGGCCAGGAAGCGGGAGTGGGTGGGTT
GGGGAGCAGGGCGGGAGGAGGGTATAGGGAACTTTCGGGATAGCATTTGAAATGTAAATAAAGAAAATATCTAATAAAAAAGTCTAGTGGTGGAGAA
TCACCCAAGTGGGAACCATCTTTGCTCCTGCACTACAGCTCTCACAGGACTGGTAGACATGCCATCTTGTTCGAGCTACCACCTCTGTCTACCACCT
ACCCTGCCCGTCATGCTCTGGCTCTGCTGGGTTGGCCGTTGACAATCTCTGGACTAGAAAGCCTGGTCAGGGAATGAACGCGATGAGAACTCGTGCC
TGCTCTAACCACAGTCTCTCTCTCCATCACCCCAGCAGAGCCAACCCTAAGCAGACTACGAACTAGAAGAGCTTCCGAGCTTTAGGTTAAACACAGC
AGAGCAGGGGAAACACAGGAATCACGAGGGATCGGGAGGGAGGGGAGGTTTTGAGGAAGGGAGGAAGGAAGGATGTGCTGACATTTGCTTAGTGTCC
ATTCTAGGGATCCACCTGTCACTQAATCCACAAGTGAGGAAGCCAAGGGTGAGAGCAGGCTGGTGTAACTGACTCATGGTGACACAGCCAGCAGCTG
TGCTAAGATTATATTCCCTACTTCATTTATACTGTGTATTGAAACCTTACCTTATTGAAGTCTGTCCTGTCTGTCTGTC1GTCTGTCTGTCTCTCTC
TCTCTCTGTTTTTTTTTTTTTTTTTTTTTTTCGAGACAGGGTTTCTCTGTGTAGCCCTGGCTGACCTGGCACTCACTTTGTAGACCAGGCTGGCCTC
GAACTCAAAAATCTGTCTGCCTCTGCCTCCCTCCCAAGTGCTGGGATTAAAGGCGTGCACCACCACACCTAGCCTTATTGAAGTGTTTTAGCAATCC
TATTGAAGGCCCATTTTACAGATGGGGAAGTAAAGCCCAGAGAGGGGAAAGTGCCAAAGCCATAAGGCAATAAAACTACTAAAGGATGTCAGCCAGT
TCTAGCTGGCCTCCAAAGGCCAATTTGCATCTGCCCTCTTGCTGCACCATTTAACCACATAGTTGCCTACTCTTATCAAATCCAATCAAATGCAGCT
AGGAGCTAGGCACTGTGCTGGGTGTACATAGCATCTCTCAAAGCACGCTGGATGCAGAGTTCTGTCCTGGGAGGAGGCACGGTCGAATGAGTACCGC
CTTCAGGAAGCAGGGAAACAGCCCGGCTGTGATAGGCTGTCCACTCCTTCTTCCCACTGGGCCCTGCTTCTCTCTCAGGCCTTCTTGGGCCTCCTGG
CTCTGCCATGGAGCCACTGGGCAACATGAACAAGTCACCTGTCTGCTGCAGCTCCTCTAGAGCAAAAGAAAGTTCCTAGATGGAGTGAGTTTTACAC
GTGCCTGGGTGAGGGGCTGAGGAAGCTACTCCACCCCCAGCTGTGGTTTCTGGTGGGGCCACTGGTGGCAGCAGGTGAGGGGAAGTATCTGTCACTC
TTCCAACAAAGGCAATCAAACAAAAGCCTCCTGGGGCATTGTGGGAACTGCAGTAAAGGCGCCAAAAATAAAACACAACTTTCCTTTGATAGGCACT
GGTAGCTGCCTTGGGGGTTGTGACAAAAGTGTTCAGATTTCACACCTCAGTTCATACCCACACACACACACACACACACACACACACACACACACAC
ACCAGTCTCACAAACAAGTCATATAATAGGCAGTCAATGATCCCAGAAGAAACATCTCCGTGGGCTGTGACACATGTCACAAQAACCACATATGTGT
CAAATGGCTTGTCCTTTCCATTTTGAAATGTTCCATATTTTAAAAGAACTTTTAAATATCCTATTTTATTTCATGTATATTGGTTTTTGTCTGCATC
TATGTCTGTGTACCCTACCCATTCACAAGGCCAGATTAGATTCCCTGGAACTGGGATTATAGACAGTTGTAAACTGCCAGGTGTGTGCCTGGAATCA
AACTTGGGTCCTCTGAAAGAGCAATCAGTGCTTTTAAAATATATATACATATGTGTGTGTGTGTGTGTGTGTGTGTGTGTGTGTGTGTGTGTGTGTG
TGTGTGTAAAATTTATTTTATGTATATGAGTACACTGTCGCTATCTTCAGGCATACCAGAAGAGGGCTTTGGATCCTATAACAGAGGGTTGTGAGCC
ACAGTATAATTGCTAGGAATTGAACTCAAGGCCTCTGGAAGAACAGCCAGTGCTCTTGACCACTGAGCCATCTCTCCACCCCTAAAGTTTTCAGTAT
GTTTTAAGCAAGGAGCTGAACGGGGTATTTTGTTCTTTGTCCTTCAAGCTACAGAACCAAACTTGCTAGCATTCATTCATAAATATTCATATGGGCA
GTATGCCTAAAAATTATAAAATACTGATAGGCAAAAGTGCATACATTGCCCACACAGCGAGAGGAACAGACACATAGACCCCCCCCCCCCACACACA
CACACACACTGGTACTACAGGGGCTCCCCTGTGTACACACACTGCAGTACAACTGCTTCCCCCATAAGGGACATTGACACCCACTCGAAAGGCCCAG
AGAGAGCTCAACCACAACCCAGTGACATGAATGGATTGACTTAGCCAGATCAAGGCACCTCGATGGCACTGAAAGGAAGGGCTTAATCTCCTTTACT
GTATCTTCAACTGTCACAGCTGGCCTCTCTTTCGGGCTATGGGCTCATCCACCAACCCTTGCAGCGGAGACTAAGGAAAAGGAGACCTTGGGGAGTA
TAGGAATCCTTGGGAGGAGAGGTTGGGAGGAGGAGAAGAACATTAGCCAAAAACAGCAGGCTGGGCGACAACGGAGGCGCCTAGTGAACTGGCCTGA
GTGGTGGTTCTGAAAGAGCCTGGGGGAGGCGGGGGCAGGTCTTGAACCCAGACAAGTCCGAGCTCCTGCTCTCACATGAACAGCCAATCCCTTGCCC
CACTCCTCTTTCTAGCTAGGTGGCCTTGGGCACATGGCTGAACTTGCTTGGCCTCAGTCTCCATCTCCACGGAATGAGAAAGTATTGTCTGCATGGC
AGGGTTGTGTGGTGCATGTTGGAAGTTGCGCCTCATCTTCTGGGCTCTCCCAACATTCAACAGTTTGCCTTCTTTCCCGGCCTTGCAATCCGACTCT
TGCTTCAGAAACTCCAGACCCCCTCATTAACGGCAGACCTTCAGAATGTGTTTATTTCTTGTCTCCCCACCCCCACCCCACTTCCCAGCCAGATGCA
AGCATGGGAGGTGGCCGTAGTTCAAGGCCCACCCTCCCTTCGGGGACTTTCAACGTTCCTCAGTTCCTTTTGATCCCATAGCTCCAGCCACAAGCCA
AACAGCCCCCAAGGCCTCCAAAGTTTTCTGCCTGTGCTCTCTACCCCTAACCCCAGTGCTGCCTGTAAGAAACCGTTCCAGGCTCTGCCCAGAAGCC
CCTCCATCCTGTTAGGTGATACCACATGCTCAAAGACTGACCCAGAAAAACAATTCTTTTCAGCTCATTGTGATGGTGCACATCTGTAACCCCACCA
CGCTGAGGAGGCTAAGGGAGAAAGAGAGAATTCTCGGCCAGCCTGGGCTACAGATCAAGACCCTGTCTCAGAAAAATTCCAACAAAATTGCTGGGGA
AATATTGCTCACCATCGGAATGCTTACATAGCATGCATGAGTGCAGNGNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNGNNNNNNGNNNNNNNNNNNNNNNNNNNNNNNNNN
MOUSE SEQUENCE—mRNA
GGACATGGACTGCTATTCTGCGTCGCCTCAAACAGGAGCTGATGTCCATGAAGGAGGGGGGGATGGCTTGCAGGATCAGATGAACTGCATGATGGGC
GCAGACTGGGCTAGCTGGAGAGAGACAAGAACCAAAAGCACAGCCTTCCTGTGTGATTTCTACAGCCCCCAGAGCACCATGGACCCAGGGAAACCCA
GGAAAAACGTGCTGGTGGTGGCTCTCCTTGTCATTTTCCAGGTGTGCTTCTGCCAAGATGAGGTCACCGATGACTACATCGGCGAGAATACCACGGT
GGACTACACCCTGTACGAGTCGGTCTGCTTCAAGAAGGATGTGCGGAACTTTAAGGCCTGGTTCCTGCCTCTCATGTATTCTGTCATCTGCTTCGTG
GGCCTGCTCGGCAACGGGCTGGTGATACTGACGTACATCTATTTCAAGAGGCTCAAGACCATGACGGATACCTACCTGCTCAACCTGGCCGTGGCAG
ACATCCTTTTCCTCCTAATTCTTCCCTTCTGGGCCTACAGCGAAGCCAAGTCCTGGATCTTTGGCGTCTACCTGTGTGAAGGCATCTTTGGCATCTA
TAAGTTAAGCTTCTTCAGCGGGATGCTGCTGCTCCTATGCATCAGCATTGACCGCTACGTAGCCATQGTCCAGGCCGTGTCGCGTCATCGCCACCGC
GCCCGCGTGCTTCTCATCAGCAAGCTGTCCTGTGTGGGCATCTGGATGCTGGCCCTCTTCCTCTCCATCCCCGGAGCTGCTCTATGCGGCCTCCAGA
AGAACAGCGGCGAGGACACGCTGACATGCTCACTGGTCAGTGCCCAAGTGGAGGCCTTGATCACCATCCAAGTGGCCCAGATGGTTTTTGGGTTCCT
AGTGCCTATGCTGGCTATGAGTTTCTGCTACCTCATTATCATCCGTACCTTGCTCTGGCACGCAACTTTTTGAGCGGTCAAGGCCATCAAGGTGATC
ATTGCCGTGGTGGTAGTCTTCATAGTCTTCCAGCTGCCCTACAATGGGTGGTCCTGGCTACAGACGGTGGCCAACTTCAACATCACCAATAGCAGCT
GCGAAACCAGCAAGCAAGCAGCTCAACATTGCCTATGACGTCACCTACAGCCTGCCTCCGTCCGCTGCTGCGTCAACCTTCTTGTATGCCTTCACGG
CGTCAAGTTCCGCAGCGACCTCTTCAAGCTCTTCAAGGACTTGGGCTGCCTCAGCCAGGAACGGCTCCGGCACTGGTCTTCCTGCCGGCATGTACGG
AACGCGTCGGTGAGCATGGAGGCGAGACCACCACAACCTTCTCCCCGTAGGGGCTCCCCCTGCCGCCGGACTACAAGGACCTCTCCAGGAGCCTTAA
TGTGGTGCACACATGCACAGACTCTCCATCCACGAATTGCTGCTGAGGGAAGAGCAATTCTGGCCAGTCAGGTTGACATGGAGGACCTAAGAAACTG
CTTAACCCCATCCCACTTATAACTACCTCAACCAAAGCTGTAAAAGATATGGCTGAGAAGTTAACACTCAAGCCAAGACAGCTATCCCCAAAACGAC
AGCCAAAAGTGAAAGTGAGAGGCTCCACACTTTCCGGAGTGAGGGATGTGGGGCCAGTGAACACCCTGGTTGAGTAGTCTTCGGAAGGCCTCTGTTG
AACCTGCTTCTAGCTTAGAGAGATGTCCCGGAGATTCAAGACAGAGCTTATCTCCACACTTAGCAAGCAAGCAAGAGATGACAGTCTCTCTCAATGC
TCCCACAGAGCACCCCTGCCCCTCCCTTCTGCCTCTCCACCGCCTTTCCTGAGGTCCAGGCCACACCATGACGCTGAGGCAGTCCCAGCTGGGGCTC
TGGATGGCAATGACAAGTAGTTGGGTCTCTATGATGGGAATAAAAGGTTAGGGGAAAGGTGACAGGAAGGAGAGAAGGTGACCCTGCTGGCTGACAG
ACGCCAGCAAGCTACTTCTTTGTTCTCTGTCAGCCAGCCACTGATACCTTTCCTCATGTTCTGCTTTTGATTCATATATCTTTTATGAAGAAACAAA
TAAAAAAAAAATTTTCCCTCGAGGAAACAACTTGG
MOUSE SEQUENCE—CODING
ATGGACCCAGGGAAACCCAGGAAAAACGTGCTGGTGGTGGCTCTCCTTGTCATTTTCCAGGTGTGCTTCTGCCTGATQAQTTCACCGATGACTACA
TCGGCGAGAATACCACGGTGGACTACACCCTGTACGAGTCGTGTGCTTAAGAAGGATGTGCGGAACTTTTAAGGCCTGGTTCCTGCCTCTCATGTA
TTCTGTCATCTGCTTCGTGGGCCTGCTCGGCAACGGGCTGGTGATACTGACGTACATCTATTTCAAGAGGCTCAAGACCATGACGGATACCTACCTG
CTCAACCTGGCCGTGGCAGACATCCTTTTCCTCCTAATTCTTCCCTTCTGGGCCTACAGCGAAGCCAAGTCCTGGATCTTTGGCGTCTACCTGTGTA
AGGGCATCTTTGGCATCTATAAGTTAAGCTTCTTCAGCGGGATGCTGCTGCTCCTATGCATCAGCATTGACCGCTACGTAGCCATCGTCCAGGCCGT
GTCGCGTCATCGCCACCGCGCCCGCGTGCTTCTCATCAGCAAGCTGTCCTGTGTGGGCATAGGATGCTGGCCCTCTTCCTCTCCATCCCCGGAGCTG
CTCTACAGCGGCCTCCAGAAGAACAGCGGCGAGGACACGCTGAGATGCTCACTGGTCAGTGCCCAAGTGGAGGCCTTGATCACCATCCAAGTGGCCC
AGATGGTTTTTGGGTTCCTAGTGCCTATGCTGGCTATGAGTTTCTGCTACCTCATTATCATCCGTACCTTGCTCCAGGCACGCAACTTTGAGCGGAA
CAAGGCCATCAAGGTGATCATTGCCGTGGTGGTAGTCTTCATAGTCTTCCAGCTGCCCTACAATGGGGTGGTCCTGGCTCAGACGGTGGCCAACTTC
AACATCACCAATAGCAGCTGCGAAACCAGCAAGCACCTCAACATTGCCTATGACGTCACCTACAGCCTGGCCTCCGTCCGCTGCTGCGTCAACCCTT
TCTTGTATGCCTTCATCGGCGTCAAGTTCCGCAGCGACCTCTTCAAGCTCTTCAAGGACTTGGGCTGCCTCAGCCAGGAACGGCTCCGGCACTGGTC
TTCCTGCCGGCATGTACGGAACGCGTCGGTGAGCATGGAGGCGGAGACCACCACAACCTTCTCCCCGTAG
HUMAN SEQUENCE—GENOMIC
AATATATGAAACAAATATTGACAGAATTGTCAAAGAAATAAGAGAAACACATAAAGCAAGAAATAAGAGAAACACATAATTCAACAATAATAGAAAC
TTAAATATCCTCCTTTCAATAATAGATACAACAACTAAGCAGTTGATCAACAAGAAAACAGAAGATTTGAACAATGCTATGAACCAACTAGACCTAA
CGTCTATCTATAAAACACCACCCAACAACAGCAGAATACATATTCTTCTCAGATATACATAGAACATTCTCCAGGATAGGCCATCTGTTAGGACATA
AAACAAGTCTCAAAAAATGTAAAAGAATTGAGATCAGACAAAGTCTGTTCTCTGACCACAACCAGTAACAGAAGGAAATTTGAAGAATCCATAAGTA
TGTGGAAATGAATCAAGGAACTCAAGGGAAATTAGAAAATACTTTGAAATGAATGAAAATGAAAACACAGCATACCAAAACTTATGAGATGCAGCTA
AAATAGTGCTTACAGAGAAATTAATAGCTATTAATGCCTGTATTTTTTAAAAGAAGAAAGATACCAAATTAAAAAAAAACTTTTCACTTTAAGAAAA
AGAATAGTGAACCAAGCCCAAATCAAGCAGAAGGAAGGAAATAATAAAGATTAGAATGGAAAAAAATGAAATATGGAATTGGAAAAACTAGAGAAAA
ATTAACAAACCCAAAAGTTGTTATATCAAAAAGATTGATTTAGTTTGATAAACATTTAACTAGACTTACCCTAATATCAAACCACATACAGATATCA
CAAGAAAAGTACAGACCAATATCTCTCATAAGACACATATAAGATAGATGCACAATTTCTTAACAAAATATTAACATGCCAAATCCAGAAACATTAC
AAAAAAAATTGTATACCATGACCAAGTGATTTATTCCAGGAATTCAAGGTTGGTTTAATATAAAAAAATCAATTCATATAATACACCATATTTAATA
GAATAAAGGGCAAAAACACTCAATTGTCTCAGTGGACACAGGAAAGGCCTCCAACTCCCTCCATGTCCCTCCAAAGGACATTATCTCATTCCTTTTT
ATGGCTGCATAATATTCCATGATGTATATGTAGCACATTTTCTTTAACCAATCTATCATTGATGGGCATTTAGGTTGATACCATGTCTTTACTATTG
TGAATGGTGCTGCAATAAACATACACGTGCATGTGTCTTTATAATAGAACAATTTATATTCCTTTGGGTATATACCCAGGAATGGGATTGCTGGGTC
AAATGGTATTTCTGTCTCTAGGTCTTTGAGGAATTCTATACTATCTTCCACAATGGTTGAACTAATTTACACTCCCACCAACAGTGTAAAAGCATTC
CTTTTTCTCCACATCACCAGCATCTGTTATTTTTTGAATTGTTGATAATAGCCATTCTGACTGGTATAAGAGGGTCTCTCACTATGGTTTTGATTTG
CATTTCTCTAATGATCAGTGTGTTGAGCTTTTCAAAATATGATTCTGGGTCACATGTATGTCTTCTTTTGTGAAGTGTCTTTTCATGTCCTTGGCCC
ACTTTTTAATTTTTTTTTCTTGTAAATTTGTTTAAGTTTTTATAGATGCTGGATGTTAGACTTTCGTCAGATGCATGGTTTGCAAAACTTTTTTTCC
CACTGCGTAGGTTGTCTGTTCACTCTGTTGATAGTTTCTTTTGCTGTACAGAAGCTCTTTAGTTTAAGTAGATCCCTTAATTTACTTATTTGTCAAT
TTTTGTTTTTGTTACAACTGCTTTTGGCATCTTTGTCATGAAATCTTGGCCCGGGCCTTGGTCCTGAATGGTATTGCCTAGGTTTTCTTCTAGGGTT
TTTATAGTTTTTAGTTTTACATTTAAGTCTTTAATCCATCTTGAGTTGATTTTTGTATATGCTGTAAGGAAGGGGTCCAGTTCCAATTTTCTACATA
TGGCTAGCCAGTTATCCCAGCACCATGTATTAAATAAGGAATCCTTTCGCTATTGCTTGTTTTTGTCACGTTTGTTGAAGATCACGTAGTTGTAGGA
GTGCAGTCTTATTTCTGGGTTCTCTATTCTGTTCCATAAGTCTGTCTGTTTTTGTATCAGTACCATGCTGTTTTGGTTACTGTAGCCATGCAGTATA
GGTTGAAGTCAGGTAGCATGAGGCCTCCAGCTTTGTTCTTTTTGCTTAGGGTTGGCTTGGCTATTTGGGCTCTTTTTTGGTTCCATGTGAATTTTTA
AATAGTTTTTTCTAGTTCTGTGAAGAATGTCAATGGTAGTTTATGGGAATAGCATTGAATCTATAAATTGCTTTTGGGCAGTATTGTCATTTCCACA
ATACTGATTCTTCCTATCCATGAACACGGAATGTTTTTCCATTTGTTTCCATTATTTCTAATTTCTTTGAGCAGTGGTTTGTAGTTCTCCTTGTAGA
TGTCCTTCACTTCCCTTGTTAGCTGTATTCCTAGGTATTTTATTCTTTTTGTGCCAATTGTGAATTGGAGTTCATGCGTGCTTTGGCTCTCTGCTTG
CCTGTTTTTGGTGTATAGGAATGCTAGCAATTTTTGCACACTGATTTTGTATCCTGAGAATTTGCTGAACTTGCTTATCAGCTGAAGAATCTTTTCG
GCTGAGACAGTGGGGTTTTCTAGATATAGGATCATGTCATCTGCAAACAGGAATAGTTTCACTTCCTCTCTTCCTATTTGAGTACACTTTATTCCTT
TCTCTTCCCTGAATATGACTGCCCTGGCCAGAACTTCCAATACTGTGTTGAATAGGAGTGATGAGAGAGAGCAACCTTGTCTTGTCCTGATTTTCAA
GGGGCTTACTTCCAGCTTTTGCCCATTCAGTATAATATTGTCTGTGAGTCTGTCATATATGGCTCTTATTATTTTGAGGTATATTCTTCATTCACCT
ACTTTATTAAGAGTTTTTAACATGAAGGGGTATTGAATTTTATCAAAGGCCTTTTCTGCATCTATGGAGATAATCATGTGGTTTTTGTCTTTAGTTC
TGTTTATGTGATGAGTCACATTTATTATTTGCCATATGTTGAACCAACCTTGCATCCCAGGGGATGATGAAGCCAAATTTGATCATGGTGGATAAAC
TTTTTGATGTGCTGCTGGATTCGGTTTGCCAGTATTTTGTTGAGGATTTTTGCTCAATGTTCATCAAGGATAATGGCCTGAAGTTTTCTTTTTTTGT
TATATTTCTGCCAGGTTTTGGTATCAGGATGATGCTGGCCTCACAGAATGAGTTAGGAAGGAGTCTCTCCTTTTCAATTTTTTTGAGTAGTTTCCGT
AGGAATGGTACTCTTTTCTGTACATCTGGTGGAATTCAGCTGTGAATCCATCTGGTCCTGGACTTTTTTTGGTTGGTAGGCTGTTTATTACTGCCTC
AGTTTCAGAACTCGTTTTCAGTCTGTTCTGGGATTCAATTTCTTCTTGGTTTGGGTCTTGGGAGGGGTGTATGTGTCCAGGTGTATCCATTTCTTCT
AGATTTTCTATGCACATAGAGGTGTTTATGATATTCTCTGGTGGTTGTTTGTATTTCTGTGAGGTCAGTGGTATTATCTCCCTTATTTTTTTAGATT
GTATTTATTTGAGTTGTCTCTTCTTCTTCTTTATTAGTCAACTAACAGTCTATCTATTTTATTAACTTTTTTTGAAAAACCAGCCTGGATTCATTGA
TATTTTGAATGGTTTTTTTTTTATGTCTCTATCTCCTTTGATTCAGCTCTGATTTTGGTCATTTCTTGTCTTCTGCTAGCTTTGGGACTTCTTTGCC
CTTGGTTTCTAGTTCTTTTAGTTGTGATGTTAGGTTGTTAACTTGAGATCTTTCTAGCTTTTGATGTGGGCATTTAGTGCTATATTTATTTACTCCT
TAATACTGCCTTAGCTGTGTCCCAGAGATTCTGGTATGTTGTATCTTTGTTCTCATTAGTTTCAAAGAACTGCTTGATTTCTCCCTTAATTTCATGA
TTTACCCAAAAGTCCTTCAGGAGCAGGTTGTTCAATTTCCATGTAGTTGTGTTGTTTTGAGTGAATTTCTTAGTCTTGAGTTCTAATTTGATTGTGT
TGTTGCCCAAGAGACTGTTTGTTATGATTTCAGTTCCTTTGTATTTGCTGAGGAGTATTTTACTTCCGATTATGTGATCAATTTTAGAGTAAGTGCC
CTTTTTGAGCCTATGTCTGTCTTTGCATGTGAGATGGATCTCTTGCAGATAGCATACCAATGGGTCTTGGTTTTTTATCCAGCTTGCCACTCTGTAT
TGAATATCTTTGTTAATTTTCTCTCTCAATGATCTGTCTAATGGGGTGTTAAAACCTCCCACTATTATTGTGTGGGAGTCTAAGTCTATTTGAAGGT
CTTTTTGAGCCTATGTCTGTCTTTGCATGTGAGATGGATCTCTTGCAGATAGCATACCAATGGGTCTTGGTTTTTTATCCAGCTTGCCACTCTGTGT
CTTTTAATTGGGGCATTTAGCCCATTTACACTTAAGATTAGTATTGTTATGTGTGATTCTTCCTGTCATTTGATGCTAGCTGGTGGTTATTTTGCAG
ACTTGTTTATGCGGTGCTCATAGTGTCACTGGTCTGTGTACTTCAGTGTATTCTTGTAGTGGCTGATAACAGTTTTCTTTTTCCATATTTTTAGTGC
TTCCTTCAGGAGTTCTTGTAAGGCAGGTCTGGTGGTAATGATTCCCTCAACATTTGCTTGTCCTGAAAGGATCTTATTTCTCCTTTCGCTTATGAAG
CTTAGTTTGGCTGGATATGAAATTCTGTATTGGAATTTCTTTCCTTTAAGAATGTTGAATATTGGCCCCAATCTTCCTCTGGCTTGTAGGGTTTCCA
CTGAGAGGTCTGCTGTTAGTCTGATGGCTTCCCTTTGTAGGTGACCTAGCCTTTCTCTCTGCTGCCCTTAACATTTTTTTTCTTTCATTTCTACCTT
GGAGATCTGATGATCATGTGTCCTGGGATGATCTTCTCATGGAGTATCTCACTGGGGTTCTCTGCATTTCCTGAATTTTGAATGTTTTGCTTGTCTA
GCTAGGTTGGGGAAGTTCTCCTGGAAAATATCCTGAAATATGTTTTCCTTTGTTTCCAACTCCCCATCTCCCCCCTTTCAGATACCCTTCAGTCATA
GATTCCGTCTCTTTACATAATCCCATATTTCTTGGAGGTTTTATTCATTCCTTATCATTCTTTTTCTCTATTCTTATCTGCCCGTATTATTTCATAA
AGACAGTCTTCAAGCTCTGACATTCTTTCAGAGGAAGATCTTTCAGAGTTCTTGCAGTCTTGGCCTATTCTTCTATGGTTTACTTCTGATTG
CATTGTGTGTTCTTGTAGTCTGTTTTTCAGCTCGATCAGGTCAGTTATGTTCTTCTTATACTTATCTTATTTTTGGCTGTCAGCTCCTGCATTGTTT
ATCATGATTTTTAGCTTCCTTGCATTGGGTTACTCTTGCTTCTTTAGCTCAGCGACGTTTTTTTTTAATTCCACATTCTGTGTCTACTTCTTTCATT
GCAGTGATCTTAGCCTTACCCCAGTTCCAAACCCTTGCTGGAGAGATGCTGCAGTCATTTGGAAGGAAGAAGGCACTTTGGCTTTTTGAGTTTTTAG
TGTTTTTATGCTGATTCTTTCTCATCTTTGTGGCTTATCTACCTTTGATTTTTGAGGTGCTGACCTTTGGATTGGTTTTTTTTGTGTTTCTTTTGTT
TTTCTTTTAACAATCTGGCCACTTTTCCATAAAGCTGCTGTGGCTTGCTGAGGGTCCCCTCCAGACTCTAGTCACTTTGGATTTTCCAGTACCTGGA
AACACACCTGTAGGAGATGGCCGGAGACCCCATTTGGGAGGTCTCACCCAGTCAGGAGGAACAGGATTGGGGAGCCACTGAAAGAAGCAATCTAACC
ACACTTCCGAAGAGAAACTATGCTATGCTGGGCACCACTTGCATCCCCAGTTGGCTTGGGATCTCCAAAGCCTGGAGACTGGTGTGGCTAAGTCGCC
AAAACAGCAAAGATGGCAGCCTGCCCCTCCCCCGAGGAGCTCTGTCCCAGGAAGTCTTCAAATCTCTGTCAGCCAGAGAACATCGACAGGAGTGGCT
GGAGGCCACAGTTGGGAGGTCCTGCCCAGTGAGAAGGAATGGATTAGGGACCCTCTTAAAGAAGCAGTCTGGCCATATTTTGGTAAAGCAGCTGTGC
CACGTTGGGAGATCCTTTCAGCCCCCAGTTGGTTTGAGCTCCCCAAAGCTGAAAGGCTGGAATGGCTGGGATGCCCAAACAGCAAAGGTGGCAGCCC
TCTCCACCATCCAGGCACCCTGTCCCAGGGAGAGATCAAAATTTTATGGGCTGTAGAAAATGGGCAGAGGTGGCTGAAGGCCTTGTTTGGGAAGACC
CACCCAGTGAGGAGGAATGGATAGGAGTCCTGCTTAAAAAAACAGTCTGGCCACGTTTTGGCAGAGCAGCTGTGCTGTGCTGGGGGATCCTTTCCAC
ACCTGGTGGGTTTGGACTCTCCAAAGTCTACAGGCTGGAACAGCTGAGTCGTCCAAACAGCAAAGGTGGTGCCCCTCACCCCCACCCCAGCCACCCC
CAGAACTCCACCTTGTCTCAGGTAGGTGCTACACTGTTGCCCGTGGCTGGCTGGAATTCCAAGCCAGTTGGTCTTTATCTGGTGAGGGCTGTGGAAG
TGAGGCCCACAGACCAATGCTGCTCAGCTCCCTGGATTCAGCCTCCTTCCTAGGGGTATGTACAGACCTCCTGCCTTGCCTGAATGGCGGTCACCTT
TGCTGGGGTTCCGGAGCCTGAGTATGTAAAGCTCCTGGGTGTCTGTGCATGCCTGAGCAGCTGCTCTGCCAATAATCCAATGCAGCTCTGTGTGTAA
GACCCAAAGGCCTGGTGGAGTGGGTTCACAGGGGATCTCCCGATCCAAAGGTTGCAAAGATCGGTGGGAGAAGCATCATTCCCCAGGTTTGCACATT
CACTCACTTCCCTGGTGGGGGGAGGGGAGGGGGTTTCCTTTGGGTCTGTGTTGCTCCTATGTAGGCCATGCCCTGCCCTGCTTTCCTCTCTCTATCC
TCTGTGGGTCGAGCTGATTCCTTAATCAGTCCCAATGCAAGTATCTGGATGTTTCAGATAAAGGTTTTGAAGTACCCACCCCTTTGTTCCTCTCTGA
GAGAGCCATGCCCCAThCCTGCTTCTAGTTGGCCATCTTTGAAAATAAAATTAAGAAAACAATTTCATTTACATAGCACATCAAAAAGAGTAAAATAC
TTAAGAAAAAATTTAACAAAGAAGTGAAAGACTTGCAGCCTAAAAAQTATAAAGAAATTAAGGAAGACCTAAATAAATGAAAAAAATCCCATGTTCA
TGGACCAAAAGACTTTGTTAGAATGGCAATACTCCCTAAATTGATTTACAGATTCAATGAAATTCCTATTAGAATCCCAGCTGGTTTCATTGCAGAA
ATTGATAACCTAAAATTCATATGGAAAAGAGAGAGATGCAGATTAGTCAAACAATGTTTAAAAAGACAGATAAAGTTGGAGCAGTCACAACTCCCAA
TTTCAAAATTTGCTATAAAGCTACAGTAATCGAGACAGTGTGGTACTGTCATAGGACAGACACAGAGATTGATAAAATAGAATTGATAGTCTAGAAA
TAAACCATThCATTTATGATGAATTGATTTCCAACAGAGGTGCTAAGACAGTTCAATGGTGGAAAAAATAGTCTTTTCAACAAATGATGCTGGGAAA
ACTAGATATCCATATAAAAAATGAAGCTGGATCCCTACCTCACACCATATGCAACAATTAGCTCAAAATGGATCAGATCTAAATGTTGCAGCTAAAA
CTATAAAAGTCTTAAAAGAACATATACGAATAAATCTTTGTATACTTGAATTATGCAATGGTTTCTTAGATATGGCACCAAAGACACAATTGACAAA
AGAAAAATAAATCAATTGAACCTCCACATTTAAAACTTTGCGCTTCTTTTGCCAGAGATTCAGGTTTTAAAAAACAAAAAGCCTTTGTGCTTCAAAG
GACGCTATCAAGAAAGTGAAAAGATGATCCATAGTAAGGGAGAAAATATTTACAGATATGTATCAGATAAGGGATTGTAACCAGAATATATAAAGAA
GTCTTACAACTCAATAATAAAAAATAAATAACCCAATTTTTTAAATGGGCAAAGGATTTGAATAGAAATTTCTCAAAAAATAATACAAATGGTCAAT
AAGAGCATCAAAAGATGCTCAGCATAGGTAGTCATTAGGAAAACGCATATCAAAACTACAAAGAGGCCGGGCATGGTGGCTCATGCCTGCAATCCCA
GCACTTTGGGAGGCCGAGGCTGGCAGATCACCTTAGGTTAGGAGTTTGAGACCAGCTTGGCCAACATGGTGAAACCCCGTTTCTACTAAAAATACAA
AAATTAGCCAGGCATGGTGGCACGTGCCTGTAGTCCCAGCTACTTGGGAGGCTGAGGCAGGAGAATCACTTGAACCCAGGAGGTGGAGACTGCAGTA
AGCCAAGATCGTGCCACTGCACTCCAGCCTGTGCAACAGAGTAAGACTCCATCTCCAAAAAAAAAAAGAAAAAAAAAGAAAAAACACAATGAGATAC
CACTTTACAGCCACTAGGATGGCTAGAATCAAAAGAATGGACAATAGTAAGTTTTGTTGACAATGTGGAACCCTCACGCATTGTTGATGGGAACAAA
ATGGTGTAGTCACTTTGGAAAACAGTTTGATAGTTCGTCAAAATGTTAAATATATAATTACCATATGACTCAACAATTTCACTTCTAGGTATATACC
CAAGAGAAATAAAGCATATGTCCACACAAACACTTGCACATAAATATTTATAGCAGCATCATTCATAATAGCTAAAAGGGAAAACAGCCCAAACTTC
CAACTGATGACAGACAAACAAAATGTGGCATATCCATACAATGGAATGTTATTCAGCCAThAAAAGGAATGGGGAATGGATCCATACCACCACATAG
GTAACCTTGAAAACACTAACTGAAAGAAGCCAGATGTGAAGGTCACATATTGATACTTCTATTTATGTGAAATGTTCAGAATAGGAAAATCTATAGA
GCCAGAAGTTAGA1TTGTGGTTGCCAGGGGCTTTCGATGACAGGAGGGGAGGGGAGCAGTCATTGCTAGTGATThCAGGGTTCTTGAGAGGGAGTGA
GAAAAAATGTTCTAAAATGTATTGTGGTGATGGTTGCATAACTCTGTGAACATACAAAAACCATTGAATTCTATACTTTAAAAAGGTAAATTTTATG
GGATGTGGATTATAGCTCAAThAAGCTGTCATTAAGAAAGGAAAAGGAAGGGAGGGGATAAGAGGGAAAGGGGAGGGAGGGGAGGGGAGGGGAAGGG
GGGAGAAAAAGATACATCGTGGCATATCTTCTTCCCTTTTACCTGTCATTATGTGTCAGTGCCTCCCATTGGCCTAAACTACCCAGAAGCCAGAGGG
AAAGCCATTTGGGACTATATTCCACTGTGACACTGAGCAGAGCAAGGGAAGGATGGGGACACTTCTCTCACACATCTCTGACTCCCCAACATCTAAC
AATGTAAATGACCTAATGATTCAGGATCCTATGACCAGCGACTGTCACCCTCTGTCACCTTTCTTCTGCCCCTTTATCTCTGAAGGCATTGAAGGGG
CCCTGCATGAGTCAGGGAGGGTTGGGGGGAGGAGCAAGGGTGGGAGGGGCAGGGAAGAGGGTGGCTTCTCCGACAACTTAAAAGGGGCTTGAACCAC
TTCCTCCCCAGAAGGGGTAGTGCGAGGCCGGGCACAGCCTTCCTGTGTGGTTTTACCGCCCAGAGAGCGTCATGGACCTGGGTGAGTGAGCCTTCTT
CATGTGAGAAGGAACAGTACCAGGGTCTTGGACACCCAGACTGACCCTGTGGAATGGGGGTGGAGGATGCGGGTGGAGCGCATAGGGGTGCTTCCTG
GAGAAATCAGTTCTGATTTGGTGGGCATTGGAAGCTACTTCCAGAGAATGCCCCCTGCTGTCCCAGGAGGCGTGCACCTGCCCTTTCAGACCTGGAG
AAGGGGTCAGCCTTTGGTGCCAGTGGGGACAAGGGAGGGGCCGAGATCATGTCTTTCTAAAACAGCAGGGACATAAGAGAGTGGCGGGGCTGGGCTT
GGTGGTCTGGCAGAACCTGTTCTCATCATGAGGGTGATAGGTTAGGCCCTCTGGGACCTCTGGGGACAGGTTTCAACAGAGAAATTCTCCCTAGAGA
GCTTAGGACCTTTTACCCACTCTCTGATTCCCACCTTTCTCCCCTGCCAAATATTCATATACCTGTCCACTTGGGGTCATGAGGAGATGCAAATGAA
ACGTGACTGGGTTGTGAGAATGTGCGTGCATTGCAATGCAAGCTCTGGTTGCTTTCTCTCTGCTGCACATCCCACCTGCTTTGATCTTTTGGCTTTG
CTGTTGCCTCTTGCAAGTTAAGGAACGGGCAGGCAACATGGAATGGGATGGCAAGGGGCACTCAGCTTGGAGTAATCATAGCCAGGCCTTCAGCACA
GCCCCTGCCCCTTAGCAAACCTCACGCACTCTGCCCCGGCAGATAACTCTACTCTTTAGAAAGCCCTTTCTCAGGTCTAACCTAGAAGGGGTCAAGG
GGAAGCACTGAGGGTGTTCTGAGTGGTTCATCCAGCTCCAGACGCCCCCTTTATAAATCTCAGCTGTGGAGTCAGACCAGGCTTGAGGCTAAGTGAC
CAGGTTCCTAGATGGCTTGTCTCACCAGCAAACAATGCACCACGGCGGCCTCAAAGCCGCAAAGGGTTGGCTTTAAAAAGTGAAGGTTCCCTGGTGC
TGTCCTCACACCCCTCCTCGCCACACCACAGCCTCCTTGATTTCATCTCTTACTTTCCCGCCTGCCCTGAACGCTGGATCTGGACCCAGCAAACAGC
TGTGAAAGGGTTAAAGTGGTCACCCCCACCACTGACTGTGAACTCCTGAAAGGCAGGTTCAGGTGCTTTGAGGATGTGAGGAGAAAATGTCTGCGGA
ATGAATGAATACACCGTATCAAGAACTCCGTAGAGTTTAGTTTTTGAAAATCTCTTCCAAATGCCTCATTTCCCAGCCAATCTACCCTTAAATCCCA
CTCCCCTGCACCCCCTCCACCACCCCCCATCACAATCCCACAGTACAGACGTCACCCACAGAAGACCCTGCTCCAGCCTTTCATACTGGCCTCCAGG
AAGGAAACCAAGCAGTGCCCAGTGGTGTGCTACTGGGCATTTGAAAAGCAGCTCCCCGGGGTGGAGCACTGGTAGCTGACTCCAGGGTGGAGGGAAA
GTCCTGAATTTGTAGTGCTTGCCAATTTCCATGGTGTAAATTCTCCCACCCTGGCTGATTGATTTCAAGCCATCTACATGACTGAACAGGGGGTTGG
GAAGATGTGCAGAGTGTCTTACCCCAGACTAGGTTTAGGGGTGCTAGACTCCTAGGCTTTGATGCAGGGGGAGAGGGTAATCTCTGGTCCAGGAAGA
AGGGATATTCTTGCCACTTCACTCAACTTGAGAGAGCCCCAACGGTCAAACCCGAAATACTGGTAGGAGGGGAAGCCGCTGACAAGCTTTACTCAAA
ACGGGCTCTCTGGGCCGTTGATGGGGTGGGTGGGCCTTGGAGGATCTGTTGTTCTGCTCTTCATCTGCCCAGCAAGGAGCAAGAGGTGGGTGAGGAG
GGAGGGTTTCTGTTACACAAAATGAAACTCCCAAGCAGGGCTGAGCTGGCCTTTTTCACGGTTTGTTGAGCAGCTTGAGGTCACCCTCCCCAACCTT
TCGAGCAATGAGACCGAAGATCTTTCATAATTCTCAAAAGCAAAGCAAGGAAAAATGAACCCATGTAGAGATGTGTGCCTCACAGCTCGGACATAAA
TGGTGGCAAAGGGGGATGCTGAAGCTAATTCAGGGGCCAGTTTCTTGCCTGCCAGAAACTTGGCTGGGGAGGAGAGGAACGCTGCAGCCTGGGTCGC
TGTTTGGAAAAATCTCTCCTCTTTGCTTCGGACATTCAGGCAACTCTCATTGTTGAGGGATTCAGGGGGTGACCAGTCTGGTGGGGAGAGTGACACA
TATGTCACAAAGCCACTGCAACAGAATAAGATACAGCATTGACCAGGGCAAGGGCAAGACAAGAAGGGAATAGTGAACCTGGGTCAGAGAGGTATTT
AAGGTGGGTTTTCAAGGGTGTGTAGGAGTTGTCAAGCCCAACAAGGAGCTGCAATGGGCCCAGGGATGTGGCAGGAATGGCAACTCCATTGCCATCA
GAAGGCCGACAAGGTAATAGAGGGAATGGGCATAAGGTGCAGGAAGTGGAGGGGAGTAGAGTGACCCAGAGGTGCATGCCCCACCTAAAGGCATTCA
AATCTATTATTTCAAAAACTCCATGCTGACAAGCAGAGTGTCCTGGGGACTAAGGCTGCAGCCGTGTAGATGGTCAGCTGAGACAACAGTAACAGAG
ACAAGAAAGAAGCAAGCGGAGGGGAGGGTGGAGTAGCTGGCCATGCCAAGGAGGAAGATGAGGAGGAAGGACGCAGGCACAGGAGACCCAATCCTTA
AAACGTGGTACGGCATCCCTAAGGAATTTGAACTTTATCCTGAAAACAATGGGGACCCATCAAAGATTTTTATTTTTCAAGTAGGTGAATTAAAGGA
TGAGATTTGTGTTTTGGAAGGAGTTCTGTGGCAGCAAAGAGGCAGCAAAGCCCGTGTCAGAACAGAGGTGGGGCCGGCTCTTTTGGGGGCCGGGATG
TTGGGGGATGGAAGCAGGAAGCACTGGTGGCCAGTGGGGCATGGGGAGCAAAGATTCCCCTCATGCTCCGTGGAGCGTCCTAGATGCAGCAGGCTCA
TCAGAAACACAGCCTGATGCAGTAGCCAGAGCCCAAACCTTGCTATCCTGTTCAAATCTCAGGTCCCTCTCTTAGCAGCTGTGTGGCCCCAGGCATT
TGGTTAATCTCTCTGGGCCTCTATTTTCTGGAATCTAAGATGAGGATCACCAGGGTAGCTCATGAGATGTTATGAGGGAGAGACACATACAGACCCC
AGTGGCCGCACAGAAGGATGGTTAGGAGTGTGGTTGCTGGAGTTGGAGGGTTTCAAAGCCTGCTCCCCTGCTGGCCATTGTGTGACTCTGAGAAAGT
TACTTGCTGTGTTTCAGTTTCTCATTGGTAAATGGGGATAATAGTAGCGCCTACCTCATGGGGCTTTTGAGAGGATGAAATGAGACCATACATGCAG
ACAGTGTGGCACGCAGTGAGAGCTCAGTGAATATGAATTATCTCATTGGATACAGCAGTTGATAACATAGAAAATGCAAGGTCTACCAGGCTCTCAC
CCATCCCTGCCTACTGTCTGAAAGTGGATTCCCCAGATCTCTAGACCTCTCACCACCTCCCTGCTTCTCCTCTGCCTCCCTTATCCAAAAAAAAAGA
GAGAGAGAGAAAAGTTGTGTTGGCAGGTGAGGAAGTTCAAAGAGGAAAAAGCTTGGGGTCGCTCTTGGGGCGAQCTGATTGGGCAACTGTGATTTCC
TGGGCAGTCTGGAGGACCTGGAAAAATGTGGGGATGGAAGTTCATGGTTTGGAGATGGGGGCAGCCAGCCAATAATTTTCTTTCCTGGATGGGCAGT
CTCTCATGTTGGGGGCTCCGGTTGGAGTTCTAGGGAATTTTCATGATGATCATTACTGAAAACTCAAGAACTCAGAAAACACCCAACTTCTAGGTCA
ACCACTCCTTAGCCCCAGAGATTCCCAACTTCATTGGTCTCACTGAAGCTGCTGCCCAAGACCAGTTTCTTTATGACTITGTAACAGACAGGGATGA
TCTGACTCAAGGCCACTGACCAGGCTCCCTTTTGCCACATACAAGAGTTCCCCTAAGTTTCCCTGTGATGTTTCTTGAAAGCCACCTGTTTTGGACC
TACAAGACCCTTGCTCATCCACCTCCTCCAAAACACCTGGAATGAGAAATCACAGGCACACACGCCAGCACTCCCCCAACTCCCCTCCCCTGACGGG
AACAGCAGGAGTGGGACAGCGTCCGGGAGAAGGGGTTATCAACAGAGTTCAACAGAAACACAGTTCTTGCTCCTCAGATCTCACCCGTTGTGACTTA
TCAAAACTTTGAGGTTTCTCATTTCTCCCTGACAGTCAGAGCGGGCTTTCTCTAGAGTAACCGAAAGACACTTACGTGTTTTAAAGAAACCTCGAGA
CTCGACCTCTAGATGAGTCAGTGGAGGGCGGGTGGAGCGTTGAACCGTGAAGAGTGTGGTTGGGCGTAAACGTGGACTTAAACTCAGGACCTAAGGG
GTAATTCAGTGAAAAAGGGGAATGAGCGGTGGGGAGCTCTGTTGCAACAGGGTCCAATCGCAGCAGGACTACAAATGCCCGAGCGCAGGCTGGGAAC
GAGGGGACAGCGGCTGCCTGTCCCCAGAATAGAAAATGCAGCTAGGAAGCCCTCTTTGAGTGGACAGCGGAGGACTGGACTGCCAGGCCAAGCATCA
GGGGCTTCATCCTCAGGGCCGGTTAGAGCCCCTGAGGATTTAGGAGGAAGGTGAGGTGGGGACTGCCATCAGAGCTAAGCTTTAGGCAGATGTGTCT
AGCAGTAGTGTGCAGGACTATTTGCTCAAGGCGGGAGAAATACCTATTTATTTATGTAGCCCGATGCAGTCTCTCCTTCCCGGAGGAACTCCCAAGC
CCAGAATCCCTGGCTTTACACAATTTCCGGGGAACACATTAACCAACTGCAACCTGGGTGCCATGCAGGGAGGCTTCCAGCAGGTGGTGCTGTGGCC
AAGGACACGGAAGCGCTGGGCTCTCACTGCTTCCCCAGCCTCCAGGAGAAGGTGCCTTAAACAGGTTCCCACGCATTTCCTGGCGCTATTGAGCTTG
GAGCTGCCAAGGGCCTGCCTTCACTTGTGGCATCGCAGTTACTGACTCTCCAGTGGGCCAGGCCCTACCTAGCTGGGACCTGAGGGTCAGGATACGG
GAAGAGGGCTACTGCCGCCCTGACTTGTAGGTAAGCTAAAACATCTAGGCAAATTACTCTGTCAACTGTCCTCCCTGCTCAGCCTCAGGAAGAAATA
AGTTCCTGCTGGGCCCCAGGCTCAGCCACAAAACCAGACTGAGCACATCTGGGCCTTGTGCTACACCGGGGTCCCCATCAGCTGCTCAGACCCCAGG
AAGCCTCCTGACTTCCAGAACACGTCGCTTCCCAAAGCTGACCCTGTTCCCCCTTTCACCTGCCCCCCAAGCCTAAGTTAGAAAACAAGTTAAAGGA
GAGAAAGTGTGTGCAGGCACTTCAGTTAAGCAAAAAAATAAAGAAGTCGTGATATCAGGAAAGAGCGTCCTTCCCCCACACTGCAACAGGACAGCAT
CCCTTCTGTGTCCTCTCGGGCCATCTTGATGCCCTCTCCCTTGGAAAAAGGACAGGCATTCGTTTACTTGTCATTCATGTATTTATTCATCATTCAT
GTATTTATCATTCATATATTCATCAGCACACAATTGTGGGCCACCTCCTCAATTCTGGGCCTGCACAAGATAAAAGGGAAGGGATCTCTGTCCCAAA
GGCAGGCCAAGGGTGTGCGCCCAGTGGACCAACAGTCAGTGCTGAATGAATGTCATTTCAGTGAGGGGGTGTTGTGGAGCTGGTGGAGCCCTGAGGC
AGTCTCCATCGCTGTGGACCAGTAAGGAACCTCATCCCTCTACCCAGCCTGTGAGGTCATCCTCCTTCCTCAGCCCACCTCCCTCAGGAGACACTCT
GGCTTGGCTTCATGCACGTGTGGCTGCGCACTCACCTGGATGCCAGGAAAATGCTCCTGGGCCAGTGGCTACCAGCACCAAAGCAGGGCTGGAAACG
TGTGCACCTACCCCACGACCTCATAGCTTTGCCGCCTCATCCCACATCAGGTGGAGTGGGAGGGGGCACTCAGACCCACCAACCAAGCTCAGTCTTG
AGGGGTGGTCTGGGCTAGAGAAGACTTCCCAGCTCCAAGAAGCAGCCCAGATGGGCCAAGACAGGGGGTGGGAGGGGCTGCCCTGCATTCTGATGCT
GCTTCTTTCCAGGACCTGGGAGTGGAGGAGGACAAGAAGGAGGTGAGGACAGTGATGCCGGGCAGCAGGCCAGGTGTTCAAAGGCACAATCTGGTTC
TGATGTTCTCTTTCAGCAAACACAGTGCCTGGAGCTTGGGAGGAAAGTTCCCAACAGCGTCTCCCCCTCCACTGCTTTCTTTAATAACAAAGACTTG
TCCCTGCCAAGCAATAACTTTCTCGCCTTGTCTCCTACAGGGAAACCAATGAAAAGCGTGCTGGTGGTGGCTCTCCTTGTCATTTTCCAGGTGAGGT
TCTCTGCCAAGGAAAGCTCTGTTCCCTTCTCCACACGGCCTCCCAAGTCAGGGGTTTAGTGGAGGAGCTGGGCATTTGCTAGAAGATACAAGACCCA
GACATAAGCCCGCCTGCCACTCATTAAGCTAATGGCTGCCCCCTCACCATCTGCTGAGTGACCCTGGAGAGCCACAGATGTTCTCTGGACTTCCTTT
ACTCCATTTAGGTTAAAACATGGGGGAAAATAACTTACCACCTTCCCTATCCCACCTCCAATCTCCCTGCCCAGGAGACAGAAATATGGACTCTGAG
TTCTTAGAGAGAGAAGAGCTGCAAAGCA1TATGTTACTGGGTTTTGTGCACGTGTGCTGAGAAAATCACCCTGGATCTGGCGATCTTGTTACT
ATCCTATCGCCCCAAAGTTGTCTCTTTCCAACCTTCACCCAAAGGCTTCCCTCGCAGCCCCCGTTTTCTTTGGTGAAATAGAGACCCATCCGGCCTC
CCTGGCAGGGGAGGGAAGAGAGGATGGGGCCCTAGGGATGGGGAGAGGGGTGGATGAGCACACACTTTATAGCAGGCCTGAACTCGGCAGGAACAAA
GACCCTCACTTTACAAAGTTCTGTTTCTCCATCCAGAAACAGACAGAAAGTGTGCCTCACCAAGTGCCAAAGTTAGAAGAAACTGTAACTCGCAGAG
TCAACTTTCTCCCATTTGTTAGAGGTGGGAGCTCAGAGTAGGGGTAGGGGAGTCCAACTGAGGCCAGAGCTAGAGAGGCAGTGCTTCAGAATTCGGC
TCATCCCCAGGTCTGGAGGCCAGAGGGGCAGCTTTCCAGAAGCTCAGGCCAGAGGCAGGCCCCAAGCTGGGCACTGTTCCCAAAACCCCATCCCAGA
GGCCCCCCGGAGCCTGGGCTGCAGCCCATAGCCTGGCCCCCTTCCCCATCACCACTCCCTCCTCTCTCTCCCCTGTCTCCAGGACTCTGGGGGGCTG
TATTGCTGGCAGTGGAGGCTGGAAGGGTGGGAAGGGGAGGAGGACAGCTGAGGCTCATTCTTCCCTTTCACTTGGGGAGGGGGTGCTCAGATAGGGC
AGGATGTGGTGGGGCTGTGGGCTGCAGAGGCAGTGAGGCTAGGGGAGGGAGAATTATAAAACAGGAACAAGGTTCAAGGGGAGAAGGGAAGGCAGGA
CCGGATGATGCAGAGTGCACTTCATAGAGGCCGGGAGTACTGCCCTCCAGGAAGGCTGAGACCCCCTCATGTCCAGGGACCCCGGGTCTAAGGCCTT
GGGCCGTAACCATGGCTGGGCCAAAGGCAGCAGCCTCCTTCAGGGCTCCCAGGCTGCAGCCATGCCCAGGAAAGGGAACAGGGGTTGCTCTGACTT
TTCGTCTCAGGCTAGCAATGGCCAAAGAAAAGGCTCCTGGACATCATTTTGTCCTCACCATGTTCCCAAGCAGGACAGTGATGAATCCAGAACTTGG
GTGTGAAAAAGAGATAGAGACAAGATGCGGTGAATCCCTCCAGCATAAATGGATAGATCCCACCGTCCCTGAGCTGTGAATCTGGTTCCTGGTGA
TAGAACCATGAGCCTCGTGAGGCAGGTCCTCCAGCCCAGCTCCCTGCCCTGCACAAACCCGTCCATCGCTGTGCGCAGAGGCAGGGGCAGGCATGGA
CCCCCTCCTTCATTGATAAATCACAGCAGGCTGCTGAACCTTCACCCGACCCTGCAGACGGCTAAAAAAAAGCTCCAGCAGCTGAGTCCTGCTGATT
GGGATCTGCCGGAAGAGATGCGAGGAAGAGAGAGGCAGAGAGGTGGGGGGAGAGGGCGGGAGCACAGAGGGAGAGTGGGAGATGAGAGCTGCGCTTC
CTTCCAGGGACACAGCTGCTGAGGAGTCCTCGCCCCCACCCCCATGGCCCATCATCCATTCACGGAGTTTATGGCACTTGGGCTCACGTTGCCTGAG
CTCCTGCTGGGTGCTAAGAGGGGACCCGGATCTAGAGTCTCTCCAGGTCACCCACCACCCATGATCACAGCAGTTTGCCCCAAAACCCAAGGCTTCA
TGGCCTCAAAATGGGAGCATTTGCCTTCCTCTCTTACTCCAGGCCCTCCTCAACCTTGACCCCCAGGTGGATTTTTTCCCCCCTAAAGCTGAGGTCT
TTTGTAAGCCCCCTGGAAAACTCCACCAAGTTCTAAAGGAGGGTCAAGAAGAAAGACACATGTTCCCTCTCTCACGGAGCTTCCAGGGGGCAAAATT
AAAGCTCCATGGAGAGGAAATGGTGCTGGAGCCTGGGCTAGGTACCTCACAGCAACCATGGGAAGCAAATAGGATTCATCTCCATTTCACAGAAGAG
AAAACTGAGGCTCTGAGAGAGAGAGAGAGAGACAGAAAGAGTTTAACCAGCTCAAGGCTCATCTGGCCCCAGAGCCCAGCTGATTTCCAGTCCCCTG
TCAGCACATGTAGAGTCTCTCAGACTTGGCAAAATCAGCCACTTGCTACTTACTAGCAGAACTGTAGAATCTCAGGCCCCACCTTAGCCTACTGATT
CAGGATAGACATGTTCACAGGATTAATCAGGTGATGCGTAQTTGGAGTTAAAATTTGAGAAGCACTGATTTAAAACCAAGAATCACCTGGAGACTTT
AGGAAAACACAGATTCCTAGACCCATCACTTGAGATTCTGGTTCAGAATTCTCAGGTGGGGCCTGAGAATTTGCATTCCTAACAAGTTCCCAGGTGA
TGCTATGATACTGATGATGCGGACCTCACGATGAAACCACTATTCCTCCCGCCTGGGCAACATGGCAGAATCCCATCTCTACTAAAAATACAAAAAT
TCGCTGGGTGTGGTGGTGGCATAAGGCTGTGGTCCCAGCTACTCAGGAGGCTTGTGGTGGATCACCTGAGCCTGGAGAGGCCGAGGCTGCAGGGAGC
CATGATTGCACCACTGCACTCCAGCCTGGGCAACAGAGTGAGACCATGTCTCAAGAAAAAAAAAAAAGAAAGAAACCACTGCTCTAGGCTAAATCCC
AGCCAGAGTTGGAGCCACCCAGCTAAACTGGCCTGTTTTCCCTCATTTCCTTCCCCGAAGGTATGCCTGTGTCAAGATGAGGTCACGGACGATTACA
TCGGAGACAACACCACAGTGGACTACACTTTGTTCGAGTCTTTGTGCTCCAAGAAGGACGTGCGGAACTTTAAAGCCTGGTTCCTCCCTATCATGTA
CTCCATCATTTGTTTCGTGGGCCTACTGGGCAATGGGCTGGTCGTGTTGACCTATATCTATTTCAAGAGGCTCAAGACCATGACCGATACCTACCTG
CTCAACCTGGCGGTGGCAGACATCCTCTTCCTCCTGACCCTTCCCTTCTGGGCCTACAGCGCGGCCAAGTCCTGGGTCTTCGGTGTCCACTTTTGCA
AGCTCATCTTTGCCATCTACAAGATGAGCTTCTTCAGTGGCATGCTCCTACTTCTTTGCATCAGCATTGACCGCTACGTGGCCATCGTCCAGGCTGT
CTCAGCTCACCGCCACCGTGCCCGCGTCCTTCTCATCAGCAAGCTGTCCTGTGTGGGCATCTGGATACTAGCCACAGTGCTCTCCATCCCAGAGCTC
CTGTACAGTGACCTCCAGAGGAGCAGCAGTGAGCAAGCGATGCGATGCTCTCTCATCACAGAGCATGTGGAGGCCTTTATCACCATCCAGGTGGCCC
AGATGGTGATCGGCTTTCTGGTCCCCCTGCTGGCCATGAGCTTCTGTTACCTTGTCATCATCCGCACCCTGCTCCAGGCACGCAACTTTGAGCGCAA
CAAGGCCATCAAGGTGATCATCGCTGTGGTCGTGGTCTTCATAGTCTTCCAGCTGCCCTACAATGGGGTGGTCCTGGCCCAGACGGTGGCCAACTTC
AACATCACCAGTAGCACCTGTGAGCTCAGTAAGCAACTCAACATCGCCTACGACGTCACCTACAGCCTGGCCTGCGTCCGCTGCTGCGTCAACCCTT
TCTTGTACGCCTTCATCGGCGTCAGTTCCGCAACGATCTCTTTCAAGCTCTTCAAGGACCTGGGCTGCCTCAGCCAGGAGCAGCTCCGGCAGTGGTC
TTCCTGTCGGCACATCCGGCGCTCCTCCATGAGTGTGGAGGCCGAGACCACCACCACCTTCTCCCCATAGGCGACTCTTCTGCCTGGACTAGAGGGA
CCTCTCCCAGGGTCCCTGGGGTGGGGATAGGGAGCAGATGCAATGACTCAGGACATCCCCCCGCCAAAAGCTGCTCAGGGAAAAGCAGCTCTCCCCT
CAGAGTGCAAGCCCCTGCTCCAGAAGATAGCTTCACCCCAATCCCAGCTACCTCAACCAATGCCAAAAAAAGACAGGGCTGATAAGCTAACACCAGA
CAGACAACACTGGGAAACAGAGGCTATTGTCCCCTAAACCAAAAACTGAAAGTGAAAGTCCAGAAACTGTTCCCACCTGCTGGAGTGAAGGGGCCAA
GGAGGGTGAGTGCAAGGGGCGTGGGAGTGGCCTGAAGAGTCCTCTGAATGAACCTTCTGGCCTCCCACAGACTCAAATGCTCAGACCAGCTCTTCCG
AAAACCAGGCCTTATCTCCAAGACCAGAGATAGTGGGGAGACTTCTTGGCTTGGTGAGGAAAAGCGGACATCAGCTGGTCAAACAAACTCTCTGAAC
CCCTCCCTCCATCGTTTTCTTCACTGTCCTCCAAGCCAGCGGGAATGGCAGCTGCCACGCCGCCCTAAAAGCACACTCATCCCCTCACTTGCCGCGT
CGCCCTCCCAGGCTCTCAACAGGGGAGAGTGTGGTGTTTCCTGCAGGCCAGGCCAGCTGCCTCCGCGTGATCAAAGCCACACTCTGGGCTCCAGAGT
GGGGATGACATGCACTCAGCTCTTGGCTCCACTGGGATGGGAGGAGAGGACAAGGGAAATGTCAGGGGCGGGGAGGGTGACAGTGGCCGCCCAAGGC
CCACGAGCTTGTTCTTTGTTCTTTGTCACAGGGACTGAAAACCTCTCCTCATGTTCTGCTTTCGATTCGTTAAGAGAGCAACATTTTACCCACACAC
AGATAAAGTTTTCCCTTGAGGAAACAACAGCTTTAAAAGAAAAAGAAAAAAAAAGTCTTTGGTAAATGGCAAATGAGCGTGTCTTTGTTTTGCAATC
CCCCACACCCQACCTGACCGTCCCCTCTCCCCTCCTCCCTGCCCTCTCCCAGGCCTCGTTTCTTAGAGACGCTCAGAACCACAAAGTTGCTCAGGAA
TGCTCTGAGCCCGTCTGGTGACACAGGACCAAGAGGCTGAAAGGTAGAAGGAAGATGATGACAGGAGGTCCATCTTGCAAATACCCTTCCAGAGTTC
AAGGGATAGGCCCCAAGCCTGCACAGTCAGCACTGAACCGGACATGCTGATCCTACTGCAGCTTTGGGAAGGCCAACTTTTCCGGGTAACCTCGATA
CCTGGGGACTCTGCATTTAGCAGCTCTTGCACAGTAGCCTGCTCACCTGTCCCAGCTCAGACCAGAACTAGAGCCCTTAACCTTTTGTCAGCCCCTT
AGACTCTGAAAGCTGGGGTTTGGAGGGAGGGCCTTTCTGTGGGCTTGGAGACCACAGCATACTGGACTAGGGGTCAGGGATGAGACCCTTTGCTGGC
ATGAGAGCTGGAACCACCCATGATCCTCTCTGCTAGCCACTGTAGCAGCAGTGGCCTCCGTGGTGTATGTCATAGTCCCTTCCTGCTGGTACAACAA
AATGCCACCGATGTAGTAATTTACAAACCATAGAAATGTATTTCTCACAGTTTTAGAGGATTGGAAGTCCAAGATCAAGACGCCAACAGATTCAGTG
TCTGGTGTGGACTCTCTCTGCTTCCGAGATGGGGCCTTGCCATGCCCTCTGGAGGGGACAAACACTGTGTCCTCATGTGGTAGAAGAGTGGAAGATC
ACAATGAGCCTAACTAGTTCCCTCCTGCTCTTTTATTGCTATAAGGGCATTAATCCCGTTCACAAGGGTGGATCCTTCATGACCTAATCAACTCCCA
AAGGCCCCCCCCAACTTCTCATACCATTGCCTTGGGGTTTATGTTCCAACCATATGAATGTCGGAAAGATACATACATTCAAACCATAGTGTGGATA
CTGACTGTGGAGGAGCAGGGAGAGAGGGACAGACAAAGGCCCCCACACAGGCCAGGTGTCCGTGTCATTTCGTGCCTTGGTGCCACCCAGAGCCGTG
ATTCTGCAAGCCAGAGAGCAGGTCAGTCCAACCTACAATTGCGTCAAGAGGGGTCTGAAGGACCCAGAGCTGCAATAGCTCTGGGCCGTGGGGAGCT
AGAGGGAGGGCTGAGGCACTCACGCAATAGGTGATTCCATCCATTTCTCCAAGTGGAAAGAGCCTCGTTGAAAATGGAGCTGCCTGGTAATGACACC
CATTCGCTCATCTCGCAAGCACGTGACATTACCCTCCTCCGAGAGATGCACTGGGCTGGAAGGTTTTTAACAAGATTTGTCAGCTGGTCTCCAAGCC
GGGAATTGGCGCCCTGAGAGGAATGAGCCTGGCACGGATGCAGTCAGCGTGGAGCGGCGTTTGCAGCAATGGCATTATTATTAAATTGGCACAGCTG
CGACTGTGTGCGTGAGCGTGTGGCCAGGGGGCAATGCCAGTGTGCGTGTGTGTCCCCGAGGGGAACCTGCCGGAGCGCATGTGCGTGTGGAGCCCGC
TGGTGTGGACATCCTAATGAGGTTACAGATGGGAAAACAACCCTCGGTTCCTGAAGGGGAAGAATGTAACAGAGCAGTCATGCAAAAGGCAGCCAGG
CCAGCCGCACAAATGCGCCCCCTTCAGCTGCAGATACATTTTTCTAGAATAACTGGACATCTCCAAATTTTACAGAGAAAACAAAACCCCCAGCAAT
TTGAGCTTGGGGTGACCCCTGGGAAGAAGGAAAAGGCCCCCTGGGAGGCCCACCCAGGGCTTCTCTCTTCTTTTCTTACCTGCTTCATACATCTGAA
CCCTCTTCCCAGGTTCACAGCCAATAAGCCTTCTCCAGTCACCTGAAATTTCCTTTTCATTTCATCCCCAGGCCCTCATCTCCCTCATCCTTGCTGG
CTGCAACCCCAAGCCAAGCCCAAGGTATTCCCAGGCCACAGGTCTTCCCTTTCAGCTAGAAGCACCACACCTTTCTCATCACAQACTCACAGAGCTC
CAAGGGACCTCAAAGATCAGCTGGGCCAATCCCCCAGTCATTTTCTAGATGGTGAAACTGAGGCCCAGCGGGGAGAAGCTGACTTGCCTGAAGAGGC
GTGGCAATGCAGGGATATTGGTTAAACTTGCTTTGGAAGCCTGAGCGTCTGGCAGGGTTGCAGGAGGGTGTGGGGGAGAGCAGAGACTCTGGAGCTC
TGCCGACTTTAAATCTCATCTCTACTGCTTCCCAGCTATGTGACCTTCAGCTGTTGCCCAACTGGTCTGAGTCTTGGGTTTCTCAACTGCAAAATGG
AGAGGATTTGTGTAAACAGTTAGCACAGTGCCTGGAATGTTGAGTGTGGTGGTGAGGAAGAGGGACTTTGCAGCCAGACTGCCTGGTTTGAACCCCG
GCTCCACTGCCTGGGCTGGTTACTTAGCCTCCCTGTACCTCAGTTTCTTAATCTGTATAATGGGGATACTAATGGTACCTATGTCCTAGGATTGTTG
TGGAAGGCACGAATCCATATATGTAAATGCTTAGAAAAACTTACATATGTGTTGTTGGTAATCTTTAGGAGATGTTTAAGAAACGGTAACTCTTAAA
AAAAAAAGTTGCTAACCACAAGGAAACCAAATTCAATTCCTTAGCCCATTTCCATCCCCACAGAATTTATAATTCAAGCTGGAAGGCAATTTAGAGA
TGAGTGAATCTGGCCTTTCCATCCATTGCATGGTGAGTCAACTCGTGCTCTGAGAGGGAAGCAACTAGCCTGAGGTCTCTTGGGAGGTCTTTTGCTT
GCTGGAACTTTAACCTAGTTCCCTGGGCTCCTAACCAGAGCTCTTCCCACCAGACCCTGCTGGGCTCACGGGAACATCCCTGCACATCCACCGTGGC
ACCCGCCCTCATCCCACGCTGTCTCGTTCTCTCCGGCAGGCACCGTTCATCTTGTCCTTTGATTAGGTCCTTAAGGAGAACCTCCCTTCTCCCCTTT
AAATAGTTCCTGTCCTTATCACTGAGTCTGGAGAATGAATGAAATATAAAAGAAATATGGCAGCGGTCCCAAAGGAAATCAGGCAACTGATCTTTTT
AAGAAAGGAAGATGGGCCGTCTTCTCTGGGAATGGTTCTCAGTTACATGTCTTTGTTGAGGGTGAACAGTTGGAGGAAAAGAAACAGACCTAAAGGG
ATTGTGGGGGAATTAAGGCAAAGGAGATGGGGAAAGGCAGGGGGAAGATGGGGCAGGAATGGGTTTAGGGAATATACACTGGGGGGGGCGGGACTTG
AAGCAAATCTTCAGTGGGTAGTTAGATGTCCCATCCTGCATGCACACACATGCATGTGCACACACACACACACACACATCACATCACACACACACAC
ACATAGAGCCTCCCCAGGTCTCCCAATCTCAGGTGAGGAGAAGGGGGCTCTATTAGTCTTAGAGACAGGTGGGCCCATGGCCTTCTTTGCAGAGAAG
ATGGTTAGAATCGGTCATGTGTCTCTGGTCTGAGACCCTAGAGAAACTCCAAGGAGAATAAAAAAGGTTGGGGGGGGGCCTCTCCCTCAACTCAAAA
GAGCAACCCTATCAGGCTTCAGAAAGCAAGCCCTCCTAGCACTCCAACCCACGAAGGAGCAGCTGAGAAAGTCCCCCAACTCCTCCAGCAGCCACCC
CCTCTGTTCCTTCACTTCTCTTGGGCCCAGGCAGAGACACAGACAGGAGTCTGTGTGCAGAAAAAAGCTCCCCCAGGCCCTTTTCTGGGAGACTGCT
CCAGACTAAGCGAATCTCAGCTGATCTCGTCGAAATCAGAAACGCCAGGATGTGTATTCCCCACGGGCTCCTCTCT5TTGTGTCTGTGCATGACCCA
ATTCAGACTCCTCTGCCGCTGTCAGATTCTCTCCTGCCCATATTCACTTTGAGGATGGATATAACCTTATTTTTGTGTGTTGTTCACTAACCCCAGG
GACTTCGCTAGCTGGGCTGTCCTACGTGGTACAGTAACCACCACATAGTGTGGCCTCCAGGTTCTCATCTCTGGGTAAATAAACATCTCCGCACAGG
CTTGGCCAGCTCTGGACTGGCCTCAGTTCCTGCATGATGCAATAGGTCTCCAGGTCACATATCCGTGCCCACTAGCAAGAGGAAGGTAAGGAGATGG
TTCTTGCCCTGAAGTCCTCTTGTTCCTGTGTCAGAGTTTATTTAGGACAGAATAGTTTATATTTGCTCCAAGTAAATAATATCTGACATTGGGAAGC
ACAGCAGATGCAGCAGATGAGATGCCCCCAAACCATTTCCCAATGTTTAAAAGAGGCCTGGAAAGGACAAGCTCATTGATCTCATTTGGTTTTATGA
AACTGCACTCAAGTGACTGAAGCAGAAACCTGACCACGTGTAAAGACTGAATGGCTCAATCTGCTTCCCAGGCCCAGAGGGTGTTTTCTGTGGCCTG
GCCCAAGGCCTATGGTGAAGCCGGTGCTAGGGGTCACCTATGTGGGAGCCAGCTCTGCCTCCTGCCCCGAAGCTCCAGAGACTTCTCCTACAACTGA
CTCTCTGGACACGCCCTCTTGCTTGCACTAGTATCTCTTACCTGCCACCTGCCCTCCTCCACACACCCCGATTTCAGGGAAGCTCAGCTGCTGAGAG
TGCTATTGACAACCTCTGGACTTGGAAATCAGGAGGAAGGGAATGGGAAGAATGGTGGCAGCTTTGTGCCTGGGCTGTTCAGGAAGACATCCCTCTG
CCTTTGTTCTCCAGGGCAAGACACAGGGCTAGACACGCCGGAATCCTTAAATCCTTTGTCAAAAGCAGAACTGGGATTTTCGTCCAAGGACTACACC
TGTCCTGTCCTCAGCCTCCTTCTCAGTTGCCCCCCCTCCCACCCGGCTCATCAGAGCCGAACCCTGAGGAAACCATGGACCGAAAGACCTTGTGAGC
GGAAAGAGACGTTGAAGAGGTGGAGGGTGTAATGAACCTAGGCGGAGACAGGCTAACATTTACTAAGTGCCCGTGATGCACCAAGGACTAGGCTAGC
TATACAACCTTTCATTGAATTCTTATGACAACAGAGAAGGAAACAGGTTTAGAGAGCTCATGTAACTTACCTGTGGTGACACAGCTAGCAACTTTAT
TAAGATTTTGTTACGTACTATACACTGTATTAAGCATCAGTATCTTATCTCATTTAATCCCCACAACAACTCTACTAGAGTCTCATTTAATAAATGA
GGAAACTTAGCCTCAGAGAGGTTAAGTAACTTTATCAATGTCACACAGCTACTAAGTGATTTCAACCAGTTCTAGCTGCCTCCAAATTTCTTGCTTT
AGTCACCQTACTTCACCGTTCAACTAAACAGCTGTTTAATACCAAATATTTCTGGAGCCCCTACTGAGTGCCAGGCACTGTGCTGTGTCCAGCCAAC
ATCTTTCAAACCCTGCCAGACACAGAGCTCTTGACCTGGGAGAAGGGAGAATCAAATAACAAAGACACAGCCCCCACCTGCCAGGGGCTGTGACCCC
ACAGCTGGGCAATAACCAGGTCTTTGCTGCTCCACAGTACCTTGTCCCTGCTTCTCCCCTCTAGGCCCTGCTTCTTACCTAGGCTTCCTCCATGGGC
TAGCCCTGCAGATGACTGAGAGGGCTGAGATGGCTTTGCCACAGACTAGTCACTGGGCAATATGGGCAGGTCACCTCCCTTTTCTGGGCCTCCAGCT
GGCCAGTGCTCAGCGGGGTAGGGCTCTGGGCATGTTGATGCTTGCACGAGGTGCAGGGCAAGTGTGTGAGGGGCTGAGGAAGCTGCTCCACCCCCAG
CTGTGGTTTCTGGTGGGGCCACCATGGCAGCAGGTGGACGGAAGTGTTTGTCACCCTTCCAACAAAGGCAGTCAAACAAAAGCCTCCCAGGGCACCA
TGGGAATAGGCGGCAAGGGCGCCAAAAATAAAACACACTTCTCCTTTGATAGGCATCGGTGGCAGGCATAGGCAGCTGCTAAGGGAGTCTGTCCTTG
GGGTGGTGAGGAAAGGGTTCAGATTTCGTGCCCAAGAACACTCACACTCACAAGAGCTGTCCCACCTCACAAACAGCCCACACATACAGATGCTCAA
TATTCACACAAGAAACACCACCGCACACATCACACACACACACACCAACACAGCAGTCACACAATCAGCATACACATATACACATACAAATAGAAAA
CTTGCCCCCATACAAGACCAGCTTCATGGGCCGCGACCTGCACAGTCACTCTGGGCCCTGCACTAAGGCCCTGCACTAAGGCCCATGCTGGGTATAA
TGCTTTGCTGCCTGCACTTTGAAATTCTTAATTATTTTTAAACAAGGGGCGCAGCATTTACATTTTGCAATGGGCCCTACAGGTTCTGCAGTTGCTC
CTGTGGGTGTGCACTCATGAGAGCGTTCACTACCTGCTTAAGCATACCTTTTAACACTTTTACACTGTTGATGGGAGTGCAAATTAGTTCAACCATT
GTGGAAGACAGTGTGGTGATTCCTCAAGGATCTAGAACCAGAAATACCATTTAACCCAGCAATCTCATTACTGGGTATATACCCAAAGGATTACAAG
TCATTCTACTATAAAGACATATCCACACGTATATTTATTGCAGCACTATTTAQAATAGTAAAGACTTGGAACGAACCCAAATGCCCATCAATGATAG
ACTGGATAAAGAAAATGTGGCACATATACACCATGGAATACTATGCAGCCACAAAAAGGAATGAGATCGTGTCCTTTACAAGGACATGGATGAAGCT
GGAAACCATCATTCTCAGCAAAATAACACAGGAACAGAAAACCAAACACCACATGTTCTCACTCATAAGTGGGAGTTGAACAATGAGAACACATGGA
CACAGGGAGGGGAACATCACACACTGGGGCCTGTCAGCGGGTAGTAGGGGGCAAGCGAAGGGAGAGCATTAGGACAAATACCTAATGCACGAGGGAC
TTAAAACTTAGATGATAGGTTGATAGGThCAGCAAACCACCATGACACATGTATACCTATGTTACAAACCTGCACATTCCGCACATGTATCCTGGAC
CTTAAAGTAAATTAAAAAAAAAAAAAATCCTACCTATATAGACAAGAGTGCATCCGCTACACACATACACACAGATGCACAAACACATGCATGCCAC
AAATGCAACACCCACACAAACAGCAATAGCCCCCCATGTTCACGCGCCAAAGCACACACATTGATACCCAGAAGGGGTCCAGACGCTCACAAGCAAC
GCTACTACATGCAGCCTATGCCCTGCAGCCTGCTGACACAATTCAATTGACTTAACATTGGGGGCAGCAGAACAATGTGAAGGGAAGGGGCTTGGAG
AGAAAACAGGTTCATGTCCTCCACCTGCAAACATCTCCCACCTCTCTCTTCGGCCCTGGGCTCACCCACCCCACTCAGCGGCTGGGACCAGAAGAAA
GGGAGAGCCAGGGAGGTGGGCGCATGGACGGAAAGGGGACAGGGATGGAGGTGGAGAGGGAGAGAGAGAGGGCAAGAAAGAACGGGTGGGGAGTGGG
GGCAGGAACTGAGtCCCCAGGTGAGCTGGCCCCAGCAGTGGTCTCATAAGAGCTCAGAAGGTCTTGGACCCAAAGCAGTAAGAACAATGCCCCTGTG
CTCCCCGCAAGCACAGGGCGGTCCCGCCCTCAGAGTCAGTCAGAGGATGCCCACCTCCCTTCTTACTAGCTACACAGCCTTGGGCACGCCGTGTAAC
TCCGGGCCTCAGCTTCCTCCTTTGTGGAATGGGATAGCCAATTCTACATTGCAGGGTGGCAAGGCACACACAGACACAACACCTCCTGGATTCCTGC
AGCAATCAACAGCTCGTCTCGTGATCAGGCCTGCAATCTGATTCTTGGCACAGGAACTCCAGACCCCCTCATTACAGCAGACATTTGGAACGCGTTT
ATTTCCCATCTTCTCCTGGTCTCACTTCACACCTCACAGCCAGGTACAAGCACAGAGGTGGATGTAATTCATGGCCTCATGGCCACCATCACTTCAG
GGACCTTCAGGATCCTGCAGCCCCATCCTGCCCCACCCCCTACCCAAACCAAGCAGGACTCCAAGGCCTGGAAATTTTTCTTCCTCCTCTATTCTTT
TTTTTTTTTTTTTTTTTTTTTTTGAGACGGAGTTTCACTCTTGTTGCCCAGGCTGGGCAATGGCGCGATCTCGGCTCACCACAACCTCCGCCTCCAG
GGTTCATGCCATTAGCCTGGCTCAGCCTCCTGAGTAGCTGGGACTTCAGGCACCCACCACCATGCCCGGCTAATTTTTTGTATTTTAAGTAGAGACA
GGGTTTCACCGCGTTAGCCAGGATGGTCTCGATCTCCTGACCTCATGATCTGCCCGCCTCGGCCTCCCAAAGTGCTGGGATTACAGGTGTGAGCCAC
CGCGCCTGGCCTCCTCCTCTATTCTTTACCCCCAACCTGTATTCTCCACCCCCAACCCTAACCTTCCTGATTTATTTATTTATTTATTTATTTATTT
ATTTATTTATTTAGAGACAGAGTCTTGCTCTGTCGCTAAGGCTGGAGTGCACTGGCTCACTGCAACCTCTGCCTCCCAGGTTCAAGTGACTCTCCTG
CCTCAGCCTCCCGAGTAGCTGGGATTACAGGCATGCGCCATCACGCCCAGCTAATCTTTGCTTTTTTTTTTTCTTTTTTTTTGACAGAGTCTCGCTC
AGTCGCCCAGGCTGGAGTGCAGTGGCACGATCTCGGCTCACTGCAAGCTCCGCCACCCGGGTTCACGCCATTCTCCTGCCTCAGGCGCCCGAGTAGC
TGGGACTACAGGCGCCCGCCAATATGCCCAGCTAATTTTTTGTATTTTTAGTAGAGACGGGGTTTCACTGTGtTAGCCAGGCTTGTCTCGAACTCCT
GACCTCTAGTGATCCGCTCACCTCGGCCTCCCAAAGTGCTGGGATTACAGGCATCCCTATTTAAAATTGTCCCAGCCTCTGTCCCTTCCCTGAAAAC
CCCATCATTCTGTTAGGTGATACCAGATTCTTAAAGAGTGATTCGGGAAAAACGAATGGGCCCTAAGGGGCATGCCAGTGTGCCCCATTCCAATTAC
AGTCACCCACAGGAGAGAGGAAAGGTCGTCCAAGTGGCCACCAACAAGAAACTATTTCATCAGATTTCTTTTTTGTTTGTCTGTTTGAGACGGAGTC
TCACCCCGTCACCCAGGCTGGAGTGCAGTGGCGCGATCTCGGCTCACTGCAACCTCTGCCTCCCGGATTCAAGTGATTCTCTTGCCTCAGCCTCCCA
AGTAGCTGGGACTACAGGCACGCAQCACCACATCCAGCTAATTTTTGTATTTTTAGTAGATACAGGGTTTCACCATGTTGCCCAGGCTGCTCTCAAA
CTCCTGACCTCATGATCCGCCCACCTAGCCTCCCAAGGTGCTGAGATThCAGGCGTTGAGCCACTGCGCCCGGCCTATTTCTTCAGATTTCTTATAG
GACATAAGTCACTCAGATCCCCTCACAGGCTTTCTGTCAACTTACTGAGAGGGAAACTGAGTCAAGGAAGAGTGAAGTGACTTGGGTGAGCAGCAGA
GTTGAGGGTTGTGGTCTTTCTGATGTGCAGTCTCCATGAGACCAGATC
HUMAN SEQUENCE—mRNA
GTGAGACAGGGGTAGTGCGAGGCCGGGCACAGCCTTCCTGTGTGGTTTTACCGCCCAGAGAGCGTCATGGACCTGGGGGTCCAATGAAAAGCGTGC
TGGTGGTGCTCTCCTGTCATTTTCCAGGTATGCCTGTGTCAAGATGAGGTCACGGACGATTACATCGGAGACAACACCACACTGGACTTACACTTT
GTTCGAGTCTTTGTGCTCCAGAAGGACGTGCGGAACTTTAAAGCCTGGTTCCTCCCTATCATGTACTCCATCATTTGTTTCGTGGGCCTACTGGGC
AATGGGCTGGTCGTGTTGACCTATATCTATTTCAAGAGGCTCAAGACCATGACCGATACCTGCTCAACCTGGCGGTGGCAGGACATCCTCTCTTCC
TCCTGACCCTTCCCTTCTGGGCCTACAGCGCGGCCAAGTCCTGGGTCTTCGGTGTCCACTTTGCAAGCTCATCTTTGCCATCTACAAGATGAGCTT
CTTCAGTGGCATGCTCCTACTTCTTTGCATCAGCATTGACCGCTACGTGGCCATCGTCCATCTGTTCAGCTCTCACCGCTCCGTGCCCGCGTCGTT
CTCATCAGCAAGCTGTCCTGTGTGGGCATCTGGATACTAGCCACAGTGCTCTCCATCCCAGAGCTCCTGTACAGTGACCTCAGAGGAGCAGCAGTG
AGCAAGCGATGCGATGCTCTCTCATCACAGACATGTGGAGGCCTTTATCACCATCCCAGGTGGCCCAGATGGTGATCGGCTTCTGGTCCCCCTGCT
CAACGATCTCTTCAAGCTCTTCAAGGACCTGGGCTGCCTCAGCCAGGAGCAGTCCGGCAGTGGTCTTTCCTGTCGCACATCCGGCGCTCCTCCATG
AGCAACTCACATCGCCTACGACGTCACCTACAGCCTGGCCTGCGTCCGCTGCTGCGTCTCCCTTTTCTTGTACTTCTTCATCTCCCCTCTGTTCCG
CAACGATCTCTTCAGCTCTTCAGGACCTGGGGGCTGCCTCAGCCAGGGAGCAGCTCCTCAGTGGTCTTCCTGTCGGCATTCCTGCGCCCCTCCATG
AGTGTGGAGGCCGAGACCACCACCACCTTCTCCCCATAGGCGACTCTTCTGCCTGGACTAGAGGGACCTCTCCCGGGTCCCTGGGGTGGGGATAGG
GAGCAGATGCAATGACTCAGGACATCCCCCCGCCAAAGCTGCTCAGGGAAAAGCAGCTCTCCCCTCAGGAGTGCAAGCCTGCTCCAGAAGTTAGCT
TCACCCCAATCCCAGCTACCTCAACCAATGCCGAAAAAGACAGGGCTGATAAGCTAACACCAGACAGACAACACTGGGAAACAGAGGCTATTGTCCC
CTAAACCAAAAACTGAAAGTGAAAGTCCAGAAACTGTTCCCACCTGCTGGAGTGAAGGGGCCAAGGAGGGTGAGTGCAAGGGGCGTGGGAGTGGCCT
GAAGAGTCCTCTGAATGAACCTTCTGGCCTCCCACAGACTCAAATGCTCAGACCAGCTCTTCCGAAAACCAGGCCTTATCTCCAAGACCAGAGATAG
TGGGGAGACTTCTTGGCTTGGTGAGGAAAAGCGGACATCAGCTGGTCAAACAAACTCTCTGAACCCCTCCCTCCATCGTTTTCTTCACTGTCCTCCA
AGCCAGCGGGAATGGCAGCTGCCACGCCGCCCTAAAAGCACACTCATCCCCTCACTTGCCGCGTCGCCCTCCCAGGCTCTCAACAGGGGAGAGTGTG
GTGTTTCCTGCAGGCCAGGCCAGCTGCCTCCGCGTGATCAAAGCCACACTCTGGGCTCCAGAGTGGGGATGACATGCACTCAGCTCTTGGCTCCACT
GGGATGGGAGGAGAGGACAAGGGAAATGTCAGGGGCGGGGAGGGTGACAGTGGCCGCCCAAGGCCACGAGCTTGTTCITTGTTCTTTGTCACAGGGA
CTGAAAACCTCTCCTCATGTTCTGCTTTCGATTCGTTAAGAGAGCAACATTTTACCCACACACAGATAAAGTTTTCCCTTGAGGAAACAACAGCTTT
AAAAG
HUMAN SEQUENCE—CODING
ATGGACCTGGGGAAACCAATGAAAAGCGTGCTGGTGGTGGCTCTCCTTGTCATTTTCCAGGTATGCCTGTGTCAAGATGAGGTCACGGACGATTACA
TCGGAGACAACACCACAGTGGACTACACTTTGTTCGAGTCTTTGTGCTCCAAGAAGGACGTGCGGAACTTTAAAGCCTGGTTCCTCCCTATCATGTA
CTCCATCATTTGTTTCGTGGGCCTACTGGGCAATGGGCTGGTCGTGTTGACCTATTTCTATTTCAAGAGGCTCTAGACCATGACCGATACCTACCTG
CTCAACCTGGCGGTGGCAGACATCCTCTTCCTCCTGACCCTTCCCTTCTGGGCCTACAGCGCGGCCAAGTCCTGGGTCTTCGGTGTCCACTTTTGCA
AGCTCATCTTTGCCATCTACAAGATGAGCTTCTTCAGTGGCATGCTCCTACTTCTTTGCATCAGCATTGACCGCTACGTGGCCATCGTCCAGGCTGT
CTCAGCGCACCGCCACCGTGCCCGCGTCCTTCTCATCAGCAAGCTGTCCTGTGTGGGCATCTGGATACTAGCCACAGTGCTCTCCATCCCAGAGCTC
CTGTACAGTGACCTCCTGAGGAGCAGCAGTGAGCAAGCGATGCGATGCTCTCTCATCACAGAGCATGTGGAGGCCTTTATCACCATCCAGGTGGCCC
AGATGGTGATCGGCTTTCTGGTCCCCCTGCTGGCCATGAGCTTCTGTTACCTTGTCATCATCCGCACCCTGCTCCAGGCACGCAACTTTGAGCGCAA
CAAGGCCATCAAGGTGATCATCGCTGTGGTCGTGGTCTTCATAGTCTTCCAGCTGCCCTACAATGGGGTGGTCCTGGCCCAGACGGTGGCCAACTTC
AACATCACCAGTAGCACCTGTGAGCTCAGTAAGCAACTCAACATCGCCTACGACGTCACCTACAGCCTGGCCTCCGTCCGCTGCTGCGTCAACCCTT
TCTTGTACGCCTTCAICGGCGTCAAGTTCCGCAACGATCTCTTCAAGCTCTTCAAGGACCTGGGCTGCCTCAGCCAGGAGCAGCTCCGGCAGTGGTC
TTCCTGTCGGCACATCCGGCGCTCCTCCATGAGTGTGGAGGCCGAGACCACCACCACCTTCTCCCCATAG

TABLE V

MOUSE NOMENCLATURE
ICSGGG CCnd1
CeleTA TnCG212S1
HUMAN NOMENCLATURE
HGNC CCND1
Celera hCG279S5
MOUSE SEQUENCE—GENOMIC
CACGGGATCCAAAACCCTCTCCTGACTTCTGCAAGCGCTGCACACACATGGTACATAGACATGCACACGCACGTGCGTAAACATGCTGGCTTTCTGA
TCCTACGGCTTCTAATTTAGCACTAACACTTAGACTTTTTATTCCCTGTTGCTGCCATGACTGACCCCAGACCCTGCCTTTCAGGCTTCTCCTTGCT
TTTCAGACATGGGACAGTTCTGGGGTCTCTTTTGCAAATTGAGACCTGTGCTCACCTAGCACACATGTGGCCTTGGGTTCCATCCCCAGAACTGAGT
GAATGAGGCATGGTGGCTCACCCTGTGATTCCAATATTCAGAATGTGGAGACAGTAGAATGAAGAGTTCCAGTTTCAGGTACACCAAACCTTGTACC
AGAAAGAAGAGGGGGAAGAGAAGGAAGAGGAGGGACAAATCTAGCAAAAGATGGAGGAAGCATACAGTGACTTTGAAGATGTGTTAACTTTCCCTTG
CTGTAAGGAAATACCTGCGGTAATTAACTTATAAAGAACAGAGGTTATGTGAACTCACAGTTTGGGGATTTTAGTCCAGAACAGGACTCTATTCTAC
TTCGAGTCTCTAATGCAGATGATGAGAGCGAAATGCTGAGCAAACAGAACTGAACTGCCCTGTACTACATGGCAAATCAGAGGAAGAGTAGTCCCAT
CGTCCTCTCTGAGGCCACCTTCAGTAACCTAAGGACTGCCAGGCTGTGCTGTTTGTTTGTTCTCTGTCTCTAGGGTGTTTCTTTTCGGGTTTGTTTT
GGTTTTGTTGGTGGTAGGTTTTGTTCATTTGCTTGTTTGTTTTTGATATAGAATCATGCTATGCAGCCCAGGCTGTTCTCAAACTTGTAATCCTCCT
GTCTTCGCCCCCTGACTACTAGGATCACAGTATGCGCCACCACACCTGGCTCTAGAATCCATCTCTTGGAAGTTCTACCACCTCCCAATAAAGCCAC
CCTGAGAATGAAGCCTTGGACAACCAGAGAATATTGCTGAGCCAAACTACAGCAAGAAGGCTGACCAGAAGGCTCTGCCAAATTCACAAGCCACTGG
CCCATCTGCAATAGATGCCATCTGTAAAGGAGTCACTTCTCCCCAGGGTGACAGATTCAATGCAACCTCAATCAAAACCCTAGCTGTGGTGGGTTTG
TGTGTGTGTGTGTGTCTGTGTGTGTTTATGTGTGTGTCTCTCTATGCATATCTGTGTGTGTGTTGGGGGTGCTAACCTGTGTCTGTGCATGCATGCA
CAGTGTCTGAGAAAGAGGGTCCAAGATGCCGGCCACTGTAGGCCATGTTACTGTTCGCATGGGACAGGCCTTAAAGAAGAGCTGCAAAAGAGCCTTG
TACCCACCTCCTCCAATGAGACCCACCCCAGTCTGCTTTCTCAGTTAATCTCTTCCTCTCTCAACTTGAAGCCTGTCTGGGAATTTGCTGTTGAGTA
GATTATAGGCTTGGCCTCAGCTGGTCCACTAAAAGCAGGAGGTGAGGGCCCAGGCCTGAGGAAGCATTGGGGAGGCAGATCCCAGGCCTAAGTTTGT
TGGAGACACATTCTCCAGGACCACAAATAGCTGACATGCTGTTACCAGATTGTCATCCCAGTACCTGCAAAGTGGGAGAACCAGAATCAGGTGTTCA
AGGCCAGCCTCTCAAAAAACAAAACTGAAGTAAGTACATCAACTCTACTCTGCCTTTTAAGAGTTAAAAAGGGGCCTGGAGAGATGACTCAGCAGTT
AAGAGCACTGGCTGTTCATCCAGAAAGACCTAGGCTCAATTCCCAGCACCCATATGGCAGCTCACAACTGTCCATGACTTCAGTTCCATAATTCCAG
GGGATTTGTCATGTTCACACAAACATACATGCAGGCAAAACAGCAATGCACATAAAAAAAAATAAATCATAAAACAAACAACAAAGAGTTAAAAGGA
CAGGAGAAGAGGAACAAGAGAAGGTTCATGTGGCCAGGTTTTCTTTTCCCCAATTTAAAAGTTTCTTCTGAGGTTTACATCTCACACCCATAAAGAG
CTCAGAAGTCAACAAAGCGAGGAGGCTTGATTCATCATGAAGTCATTTCAAAATTTCTCTTTCTGAATATAAACCAAAATATTTAATTAATTCCACA
TACCACAGGAGTACATTACACCCTTCAAATTCACTCTGCTTAACAGTAAACTCGTGAACGAATGCACAGAACAGACCCCCAGTCACCCACCAGCACT
ATGCTAAATGTCCTATGCACATTACCTGAATTACTTCTGGCCATGGCCTGGCAGAGGAAATCATCCCACTGCCCAGCAGAGATGAGGGCATCTTTGC
CCACAGCCTCTCCAATGCCAGAGAGCACAGCTCTGTACGTTGCCTCCTGTAGTTACAGAAATGTTTTCCAACTTCCTCGCATCCTGAAAAGAAGTGG
CTTGCCTTAAAGTGACTTTTCTTTTTCCTTTTCACAAACAGTGCAGGATGGCCTCCTGTGCCAATGACAGGTATGTGCTACCTCATCTAGTTCTGGT
AGTACGTGGATTTTCTCTCTCTCTCTCTCTCTCTCTCTCTCTCTCTCTCTGTGTGTGTGTGTGTGAGAGAGAGAGAGAGAGAGAGAGAGAGAGAAAG
AGAGAGAGTGAGAGAGAGAGTGTTTGTGTACCTGTTCTTTCTTGTGTATCTGTGTACCTGCTCTTTCTTGTGTGCACATATGTGCACCTGTTCTTTC
CTCTGTGTGTATGTATCAGCTCTTTCTTGGGGGGGGTGTATGTCCCCCTGCTTCTGCCTTACAAGGGCTAGTATCTCAAGCAATGAGTTTGGTGGAG
TTACAGATGCCTGTGTCCAGCTCTCTATGGATTGTTAAGCTGCTAACCTCAGCACACTGGTTTTCTGCCCTGTCAAATGCCTGCGATGACAGCATCC
ACCTGGTAAGCTGGAAGGTGGGGTCTGCTCCTCCATCCCACCTGAGAGGGAATGATTTCCTTAGATAGGCTGCGGCTGGAACTCACCTGATCTAGAA
GTACACTGGGGGCACACAGGAGCCATGGAGCCCCTAAGCCCCACCAGCAGCTTCTGTCCTATCCCTTCTGGCAAAGGGGCCATCTGTCAAAGGAGGG
GTTGAAAGATGGCCCAGAAGAGGTCTCCTCAAATCTCTGACCCTTACAGGACTCTGATGATAAATGATACAGTCTCAGTAGGCCTATCCTGGCTTCA
TTTCTTCTGCTGTGATAAAATGTCATTGGGGGAAAAAAAAAAAAGCAGCTTAGGGAGAAAGGGCTTATTCTGGCTCGGAACTCCAGGTTTCCAGTCC
ATTACTACATGAAAGGCAGTGTGGCAGGAACTTGAAGCACATGGTCACATCATATCCTCCCAGAGCAGTGAGATATGAATCCAGGGGTGGGGACTGG
AATGATGGCTCAGGGGTTAAGAGCATCGGCTACTCTTCCAGAGGATGCCGATTAAAACTGTTTGTAACTCCAGTTCCAGGGGATCTGACACCCTCAC
GCAGACATACTTCAAATAGACATACATGTGGGCAAACACCAATGTACTTAAAATAAAAATAAATAAATTATAATAAATAAATAGGTGCTTAGTTACT
TTTCCCACCTTTAATCAGTCCAAGACCCAAGCCTAGGGAATGTGTCACCCACTCCCACACTGGGTCCTCTCACATCAGCTAAGATAGCCAAGACACT
ACCCACAGCCGTACCCATAGGGCTTCTTGAGACTCTCTAGCAACGTGAATCGAGGTTGTGTCAAGTTGACAATTGAAACTATTATGGTGCTTAACTG
CTCCCTGATGAACAGGATGTGGCAGAGACAGGCATCCCATTGGTTAATGTCAGAAGTGTCATCCCTCCGTGGACTGAATTTTTGCCTTTAACTTATA
TTTATTTGCTTGTTTGTTTATTAGTGTTTGTATCTATGTGCATGTGTGAGTGCGGCTTCCCTCAGAGTCCAGAAGAATGTGTAGAATCCCTCTAGAG
CTGGAGTAGGGGGCTGCCGGGTATGGGTGCTGGAAATCAAACCCGGTCCTCTGGAAAAATCAGCAAGCACTCTGACCTGTTAAATCATCTACCTAAC
CTCATCCTTGCTCATGTTTTAAAAATAGTTATAGTGGCCAGGTGGTGGTGGCGCTCGCCTTTAATCCCAGCACTTGGGAGGCATTGGCAGGAGGATT
TCTGAGTTCGAGGTCAGCCAAAGTGAGTTCCAAGACAGCCAGCAGGGCTATACAGAGAAACCCTGTCTCAAAGAACCAAAAATAAATGAAATAATTA
TAGTGGGTTTTTGGACATTCTCTGTATGCTAAGCACCTTCCAGATGGGCTTCCTGACAAGAGATCCGTGGCCTGACCCACACTGATTTGAAATAATG
ATGAAAATTCAACTTTCAGAAGCAAAGGCTGTAGTTCCTCTCTGGCTTCTACCACAGTCAGTAACCAGTCCAGTTCCTTAGAGAGTCAGTCACCTTA
GTACACATTCCAGCGGAGCCAAGACAGTATAGTGCTCCTTATAAACATGGAAGGGCAGTTCCTACCCAGCGAGTCACTGCCGGCTTGGTGCCTCTAC
TGAAGCGCAGCCTCAACCACCCTTCACCGCGGGGAGCGTCCTCAGGCTCTCGCGAGCCACTGCTGCGCGTCGCGTCCAAGGTTTACGGTAAGCGCGG
CGCTCAGGACGACCACGTGGCGGCGAAACACCGGCCGGCGGGAATGCGCGGAATGCGCGGTGCGGCCTCGCGCGCTCCCCAAGCCGTGACCCCGGAG
TTCCCGGCGCGCGTTTGTGGGACCGCGGAGGCTAGTGCCATGGGTCCGCCCGCGCGTCCAAGCCACCAAGCACCACAGGGAGCAGGGGCCCCATGGA
GGGCTCAGCACGTGAGGCTAGACAACCTTTTCCAGGACCTGGGGCTAGGTTTGCCCTCCCTGTTTGGGCTCCATCACAGATCTGGAACCCGCTTAGT
CCCCATTCTAAAGCCCCCACTGATCTGGATTAAGAATGGAGATGCCTGGTTTTGCAGGGAAGCAGGTCCCTGGGCTCTGCGCCCTGACCGCCCTTCG
GAGTGCTGAGGGACCAGTGCCAGGGGCTGAAAGACCCCAGTTCCAGCAGCTGCGCCCTGGGATCGCATGAGGGTTCGCTCACCACGATTGAGCCACG
TGGTCCTGCCTGGCCACTGGTGGCTCACCTTGGCCCGAGTGCCCGCTCCCCACCATTTAGTCTGGGAAGGGCCGAGGGCCAAAGCCCATAAGGACCC
TAGGGTTCTTCAGAGCACCTGTGATTCGTTCAGAGAGCTGGAGACCGAGCAGACATCCCTCCTTCAAATTGCATCTCTACCCTTGGGGGTCCCTGGA
GCCAGGCTTGCCGCTCCAGAGCTCATTGCACGGCGCGCGTCTATTCGCTCTGGGTGGTGGGATTTAGGGACTGAAACTTATTTGGTAAGGCACGCTT
GGGAAGATAGGATGCGACGGTCCCAGGTGTGCACAGGGGATGAGCTGAGCATACACACCTTAGCCGAAGGGTGCCTGAAATCCGCTCAGGGTAACCT
AGGCGGAGCAGCCGTGTAGCACGTGGGCTGCCACGCGCGCCCCAAAACGCCTTCTGGGTGAGGAGAGGGGAAGCCGTTTGCCTCCGGAACCTTGGGG
TCCATATTCGGAAGTGTTTTCTCTGGGCGACTTGAAGGCTAAATTAACAATGGCTAGCTGGAGCAGAAACAGCCAAGTCCTTTTCAAGTTGCGCCAG
GTATGCGGCTGCAGGTGACCCCACCTAGGTGCGTCCGCTCTTCTCCAGGAGGGGCTACAGCCAGAGGCTCAGTATGCCGCCCAGCCCCGCACCCCCT
AACCCGAGCCCCGCGCTTAGTCGCGAGGGTTCCTCAATGAACGCGCTCCCTCCCACTTCTCAATGAAGTTCCCACAGCCAGGGATAGTGGCAAACGC
AAGACTAAATCTCCGCTCTCTTTGGAACCTTGGCTCCAGTCAGGTGGGGGTAGGGGTAGGGTTCGAGGAGGCAGGAAATCCGAAAGGGGATTAAAGT
TACTTTGAGATTTTCTTTCCAAATAACGTGGTTCCCTCTCGGCTGGGCAAGTGGCCTTTTGTCTCCAAAAGGTCACTGACAAATAAGCGCAGAACAC
TTTAAGCCCAGAGCGGACAGTTCCTGCCACGAGATTTTTTTTAAAGCCAATGTAAAGTTAAAATTGCAAAAGAAAGTGGGTGTTTGTGTTTAAGTTA
CTATTTCGCTGGAAAATAAAAATCCAAGTTCTTTCTCTGAGATTCCTAAAGCAGTTGTCCCGGAAAGAACTCTGAATTGCAGCCCACCTCCTGGGCT
CGGGATTCGGACAAGCAGGGTTTCCAGGATGGAGGATGGGCGCTCCCCGCCGGGTCCCAAATTCCTGCGCCTCCCCTACGGGTTCCTGGACGGCTCT
ACACGCCCGCCAACCGGACTTGCACTTTGCTCCTCTCCCTGAGCGCAGAGCTCAACGAAGTTCCTCGTGGGAGATCTGCCCGGTCCGCCTAGTAACA
GCACTGAGTCCGGATTGGCTCATGCAAATTTCAGTTTCCTGGTCTTTCCCGCGGTGGCGGTGGTGGGGGGTGGCTGCGGGTCGCTAGCTCGTGCTGC
AGGTGGGCGAGCCCCTTTATGCCCCGCTGCCCTTGGTGGCGCACCCTGGCTAGGCTAGCCTGCTCAGTTCGCAGGGCACTCAGCCGCGGGTGATAGC
GCGCAGCCCTATAAATCATGCTTACTGCCGCCCGGTAGGGATTTTATGAATGAAAAGGCAGCCTGGCCGCCCTCGTGCTTAGGCTAAGGCTCCCAGG
CTTGGCGGGGCCTGTGGCCATAGCAGCGTGGGCACTCTGCCCGGGCCACCCCACCACCTTCGGAGCTACAGTGGAATCACTGTCCCGAAGGGTCCGG
ACTTTAGGGACCCCAATGTCGGGGGAGGGGGAGCGGAAGGCATGAGCTCGGAAGQTGAGGGGTTTGCGTCTTGTGTGCCGCCTGGACGCCGTGCGCT
CCTTTACCAGTTTCTTCTGGGACACGAAAGTATTCCGTGGGAAATGTGTGTGAATAGTTCGCCTAGCTTGAAGTCGGGTATTGTGAAAACGTTTCCA
AAACTAGGGAACTAAATAATGGACGAAGGTGGTGGAACCGCTTTATTCTAAAAATATTTTATTTGAATCTATAAATTATAAACTTAGGAGTCCATTC
AGTTAACCACGACCATCTATAGATTCTCTTTAAATATCACCTTATCGGCTCACAAGTTTATCTTGATTCTCACCAGCCGCCCCCCCCCCCCAACTCA
AGACTGCAATTCTAAAGGTGGAGAAACACCACCACCCTCAACGAAGCCAATCAAGAAGCTTCCGGTTGTCTGGTTCCTGGAAGGGCGACTAATAACT
TGCAGCGATTTACTTTTCTTAATTAAAAAATAAATTAGGAAGGAGCCTATCGTGTCTCAACCTTTCTTTCAACGGTTTATTTTTCTTGGGCAAACCG
CCTGCAGTGGAGGAGGGGGATACTGGGGGACCCTGGCCAGGATAAACCGGTCACTGTATGAAGACAAATCTCAGATCCCACCCCACCCCCCAGCGAG
GAGGAATAGATGAAATAATGGCCACCATCTTGAGCTGTTGCTGGAATTTTCGGGGTTTTATTTTATTTTTGAGCGAGCGCATGCTAGGCTGGGGATC
CTTTAAGTTCAGATACCCCTCTGGCCCTTTGCAACCACCCCAGTGCGCCAGGATGGAGCCTGCACGAGAGTTTAGGGCTCGTCTGGCATCTTTCGGG
TGTTACACAGTTCCTGAATTTTACACGTGTTGATGAAATTGAAAGAAGACAGGGACGCTGGGATTTCTAAGCAATGGGTCCGCCTGTGGGTGCCTCG
TGGCGTCCTCGGAAACGCACCCATTCTCCCGGTTTAAGAACAGGGTGTCCTTGCACCCCCAGGCTCCCCTTCCATACATTCTTCCTTGGCTTGCGTT
TGGCCTGGCCTCCCTCCTGCTGTCCTCCTGTCCAGAGCCGCCACTACCCCACCTCCACAGGGTCTCGGAGGACCCTCTTAGGGAAAGAAGCCCCCCT
CCCCCTTGCCCCGCTCTTTCCCAGTTTGGAGAGAAGCAGTCCGAGCGATTTGCATATCTACGAAGGCTGAGGGGGAAGGGNNNNNNNNNNNNNNNNT
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNCTTTATTGCAAAGCAAAAAGTGTTTATTAATAATTGGGGCAGGGCAGGGAGCGGCAG
TAGGGGCGCTGGGACAGGCACGAGGGGCCTCGTGGCGGCCGGGAATCACTAAATAAGACAGTGTGAGGAATATTTGGGGTGGGGGATTCTTTGTTCA
AACAGCGGACCTGGGTACCCCGCACACGCTGCCTGGCCCAGAGAAAACGCCAGGCTGCAAGGTCGCGTGGCCCCCTAGTCAATGAGACTGACTGTTG
TGCGTGCGGTTCTGGATTTGGAGGAACCTGTCTAGGTCAAACTGGGGGCGGAGGGGGACAAAAGCACATTTTCAGATGTTCAGCAGGAGACTCAGGG
GTCCCAAAATATTTTAAAATAATTTTTAAAGTGAGGCATAATGCCCTTGTAGAGGCAAACAGCGCCCGCACCCTGCAAAAGGGGGGCCCCCCGGAGT
TTGAGTTGCTGGAGCTCACCCCCCAATGTCCACTGAGCTCCTGACCCTCGGCTTGGATCAAGGGTCGTCCGATGGTTATTTTCAGGTCGTTTTTAGG
CACCTAGTTATTTTTTAAATATTTTTAAATATTTTTTGAAAAGATGACGTCTGGGAAATGCAGCGCGGCGGCCTGGGACGCCACCATTGTGTCTCGC
GTGCGCTCGAGCGGCGCCCAGCCCCCGGCGCCCCGCCAGCCCCGCGGTGGGGTTCTCTGGCTTGTGGCTTCCTAATTTCAACAGGGGACCGGGGTCT
CCGTGGCCACCATTGCTTCTACCGGCCCCGCCAGCTGCCCAAAGACTTTCCCTTTCAGTTTCAGGGTGGGIGGGCACTGGGAACCCTCGGAGCAGGA
GCGCATCCCAGGGAGTTGGCCACCGGAATCCCTCTCTGTGCAATCTAGGAAAAGGGAATGGGGTCGTATCGAGCGACGCCCCCCTCCCACGTTGACG
GTCAGGGTCCGGATCCCCGCAGGTCTGTGAGGAGCAGAAGTGCGAAGAGGAGGTCTTCCCGCTGGCCATGAACTACCTGGACCGCTTCCTGTCCCTG
GAGCCCCTGAGAGAGCCGCCTTGCAGCTGCTGGGGGCCACC1TGCATGTTCGTGGCCTCTAAGATGAAGGAGACCATTCCCTTGACTGCCGAGAAGT
TGTGCATCTACACTGACAACTCTATCCGGCCCGAGGAGCTGCTGGTAACCACGTAACCCACCACCGACAATCCCTCTTTATGCTGGGTGGTGGGAGC
TGCAAATGGCAGGAGATCCAGCCATCCATGGACAGATCTCCTGCCCCACGAAAGGCACCCGCACCGCACCGCACGACACCGAAATGGCAGTGAGCAG
CCCCCTAGCGACGTGCAGGCCACGGCGCTGGCGGTGGGAGATTCTTTTTGTCCGCAGTCTGTCCCCTTAGTATTTCTACAGGCACATTTTGTTGTTT
ACCTGTATGGGGTCCCGGGTTGACCCAACTCTTTAGACCTTCTCGAATCAGCCTGCCTTCCTTTTGACCGTGTTCCTCATTCTCTGCAGCAAATGGA
ACTGCTTCTGGTGAACAAGCTCAAGTGGAACCTGGCCGCCATGACTCCCCACGATTTCATCGAACACTTCCTCTCCAAAATGCCAGAGGCGGATGAG
AACAAGCAGACCATCCGCAAGCATGCACAGACCTTTGTGGCCCTCTGTGCCACAGGTAAGGCTCCACCCCCATTCTTCCCAGGAAGAGCGGGCTCCT
TGCCACTCCCCCTGGGGTTAAGGCCTCTTTTCCTTGCTATCTATTCTGAGCCCAGGGTGCAGATAGTTTGTTGGAGAGATATATGATACTGGGTGGT
CATTCTCTTGGCTAGTATCTTGGAGACCTAACAGAATGACACAGCTTCCCTGTGCCAACCTTTCCACTGCCAGGAAGTGGTACGGCCACAGCATGCT
TTCTGAAGAGGTTTTTGCCTCTTTTCCTAGGCTTCCTGCCTTGCCTCTGTTCTTAAGGAGCTCGAGCTATAGAGCACACACACACACACCAGCTTCT
CCCAGCTCTGAGCCACCTGCTAGGGGTTCCTACATGGATGGGACAAATGGTCCAGCCTCAGCCCACGAGCTACATACAGTACAGCCCCAGAGCAGGA
ACTTTAGAGGCCCCACCAGGGGTACTGCAGGCCAGTACTTTTTGGCTGAGTCCTGCCTGGGAGGGTTGGCTGGAAACAGAGAAACTAGAACCTCCCT
ATGACTCCTCCAAGGGACCCTCTCTGGCCCCAGACCCCAGGTAGATTAGGCTGACATAAGTCATGGTAGACAGCCATGCAGGAACCCACACACATAG
ACAACCCTGCTACTTCTGAGTGGTTTCAGTACTACTTCTGACAGCTGCTCCTCACCTCTAACTGAGAACCCTACCCAGGAGGCTTAGCAATATTGAG
GGGGTGTGGTGTTCTCCCACCTGTTGAGGACCCCCTTCCTCTGTGGCCCTGATCAGACCTCAGTTGCTATAGCTGTTTGGCCTGTGATGTCAGAAGG
GCCGGTTGAGTATCTCCCCCCACCCCCAACCTCCACCTCACCCCCGCTGTCTCAGTGACATCTTCTTATGTCTGACAGCCCCATAGCCAGTGTTTCT
AAGCTGCTGGTGTGAGAATTTGGAAATTCTAGCTTGCCAACTACCTACTGAGAGTGCTGAGCCCGCCCCAAGGGTACCAAACAAGGGGGTGGCAGAT
GGGAGTAGCAAGGCCATCGGTAGGCTGGCAGGATCTAAGGGGTGGGGGTGGCCCTGCTGTCAGTCAGGGAGGCAGCTGGCTGGGGTTCCTCTGAGGA
GAGCGGAGCCTTTGGAAGCTCCAGGCAGCCTTTTATGGAGCTGAAAGTGCCTTCAGAGAGGCAGCAGAGTTTCCCAGAGATAACTTGGGGATGGAGA
GCTGGACTCAGCTCTTCCAGCTCTCCTTACTGCCTTTGTGCCCCCTGGAAATACAGGTGGGGGGTAAAAGGACAGCTTGGTGACCTGCGGTAGGCCA
GTGTTCTGTGGTCTGGAGCATTTGTTTCTCTGTAGGAGCAACCAGCTCCTTTGTCACACGGGAACCACTTTTGGGAAGAGGCCAGTGTGACATCACG
TGAATGGAAAGATGCTAACATCAAGGCCAGGAGATCTGTCGTCTGTGTGACAGGGGTTCCCCCTATAGGAGATGGGAAGGCATAACTTCCCTTCTGC
AAACCGCCCCCCCCCCAACCTGGATTCCTGACTGCAGTTGAACAATGTGCTAGCTAGGGTTGGGGTTGTTAACACAGGTGGCCAGAGCGCTGTGTGG
ATGGATACAGACAGTGGGAAACTGAACTTAATGAAGTATGTAGGTGTCAGACATGCCACTAAGCCAGGGTACACCTATGTCATCAGCAACCAGTGTT
AGTAGCTAGGGATCCTTATAGGACATAACAGATCTTGCTACTGCCCGTGGGAGGGAGAAAAACAGGCTCTGGAAGGGGCTGGGGACTCCTGGTCCTG
CTGGCTGGAGTCTCACTCCCTGTAAAAGGGCTGTTTGTGATTTGAACTTGACTCCATAGCCAGGTTTTGGTTCACTGGACTCACTAGCATGAACTGT
CCTTGGTGACACCTGGAGGTATTGCTTGTCTGCTGGATCCCTTTAAACAGCCTGTATGTTCTCTCAGGGGTGGTCCCCAGAGGGGGGCCACCTCGCT
ACAGCAACTCTCTGCCTTCCTGATCCCATTCCTTCCAGCCTCCTTGATCCCAGGCTCTGGGTGACACACAAGATGATTGATGGGAGCTGACATCTGT
TCATTCTTGTGTCCTCCTCTGGCCTGGAAGCCCTGATTCGCAGGCCAAGTGACCAGCTGTCCCTGTCACTATTTCTTTGTTGGACGGGACAGGGAAG
CAGTTTTCAGACTCATCTTTGGTGGGTTTATGCGGAGAGGCATTCCATCGGTGTGACATACAACTGCAGGATGCTGAGTCCAGTGGACCTGAGAGGT
GTGGAGTTGGTGACCCCAGATGAAGTCCTAAAATGACACCCCACTACTTCCCAGTTGGGACATGTACATATGAGAGTCCCTAAGACTCCAGGGCCCC
TCATGAGGGACCTTGGCCTGGAGATCTGAGGTGGCTATTCCCAGCTCCCTCTGAGTCTATTCCTACTGGGTTTTCCTGGACTGGGGTCACATGTCTT
CCCCAACCTGGGTCAGTAAGCCGGGAAGGCTAACTCCCTTCTGTGGAGTCTAGCCTGGCCTCTGAGGAACCTGGGCCTGTCATCTCTACAGCTTTCT
CCTGTGTCTGAGATGTGTCTCTAGTCCTTGGGGTGCTGCCAGGCTGACTCTGGAAGAGACAGAGATCCCTGCCTCCCACCTCAGCCGCGCAAATACT
GGGATCCAGATGTGAAAGCACCACACCTTCATAGATTCCATCCTCTTAGGGCGACATAGCCAACCCCTCCTTGATGTTTGCTGGAAGTGCTGGGGAT
TTGAACCCGGTGTGTATGCCAGGCAGCATTTAAAGTTTGAGCCAAGGCCTTATTGACAGGTAGCATCAAGGCTCTGGCCTGGACACCGTAATCCTCC
TATGTGCTGGGGTCTAGAGATGCAGCACTGTGTGCACCTAGTGCCACTTAGGTGTCTCCAAGGGAGCTAGAGTTGATGTGGGCATCCATTGGAAGGG
CTTGAAGACCAGCCCCGAGGCTGTGGGTGGGAGCTGAATCATAGGCATGTCTCTGTCCCTCTCGCTGTAGATGTGAAGTTCATTTCCAACCCACCCT
CCATGGTAGCTGCTGGGAGCGTGGTGGCTGCGATGCAAGGCCTGAACCTGGGCAGCCCCAACAACTTCCTCTCCTGCThCCGCACAACGCACTTTCT
TTCCAGAGTCATCAAGTGTGACCCGGTGAGTAAGTCTCATCAAAGGAGGCTACAGCTGGCAAGGGCCTGGGAACCTTGGTGGGCAGTGCCAAGGCAC
TGTGTGACTGGGCTGGAGCTGAGAGGTGCCAAATCTGGGTCCCCACGAGCCTCTTCCCAGAGTCATGGGTCCCAAAGGCATAGAGGGCTGTGGTGTG
GTGACAGTGCCCCTGCAATGTGTATGGCTAGTGGCATGTGAAGCCCGGTCTGGCCCAGCCAGCATAGGACCATATCTCCCTCAGTGCAGACGTTGGG
GATTGAATCCATAAAGGACTCTGGTATAGACCTACACATTCTAGAACTTTCTTTGTGTGAGAGAGGGGTTTGCATCAGGCAGACTGTGCTACGTCTA
ACTGTACCTTTCTGCCAGGAGTACAGCTGATCATCCTTGGGGCTCTGGCCGTGAATGTTTTTCTCAAGGGCATCACAAGTTGGCATAGACAGGCAAA
AGTCCAGTTCTGGCTTGAACTTCAGTTGGGGGTTCAATGGAGGTCAGAGCCACTGGCATGTGGAAGGCTTCCAGGTGTGACAGCTGTTCTGCCCTGC
CTGGCATCAGCCACAGGGACACCCCGTGCCCCATTCCTACATATCTCAATTGTAGAACATGCAGCCCCCTTCCCCTAAGTGTCTGGGGCTCCTGAGA
ACTGAGAATAGGGTATGTGGGCAGGTTGGAGCCCACAGGCTTCAGAGAGGGAAGGGTGTCTGTGGGTCCATACTAGGCTACTTGGGCCTCACTTTCT
CCATCTGTGAATTGGGGCCATTGCCCTTCCATCCTCTAGCCATGGTGTTCATCAAACAAAGGGGGAACAATACACAAGTTTTGAATGTGGGGGCTTC
CCAGGTGTTCCCCAGGAATCCTCCTGGCTTCAGGAATATAGACAGTGCCCTAGCTTCAGCCTGCCTAGGCAGGGCTTCAGTTCATGAGTCTTATTCC
TGGGCAAATGCTTTCCAGACCCTACCTGTGCTGGAGCTGTCCTCTCTCAGAGGCCACCCAAGGCAAAAAGTTATGAGAGATAGACGCTCCTTGTGGG
GAAGTTCAGGGTACAGCCTGGGAGGAGGAACCTAGAGAGTAGCAGTACCTTTCTAGTCTCTGGCAGCTCTCACCGGATTTAGGGGTGGCTTGGGGAG
TTTCCTCAAGCCTTGCTCTTAGCAAAGCCTGCCTCGCAGGTCAAGGCCCTGGCCACCGGCCTCTGGCTAAACAAGGACCCCCTCCATCTCCGCCCGC
CTCTCCAGGACTGCCTCCGTGCCTGCCAGGAACAGATTGAAGCCCTTCTGGAGTQAAGCCTGCGCCAGGCCCAGCAGAACGTCGACCCCAAGGCCAC
TGAGGAGGAGGGGGAAGTGGAGGAAGAGGCTGGTCTGGCCTGCACGCCCACCGACGTGCGAGATGTGGACATCTGAAGGCCACCGGGCAGGCGGGAG
CCACCAAGTAGTGGCACCCGCAAAGAGGAAGGAGCCAGCCCGGGTGCTCCTGACGACGTCCCCCTTGGGGACATGTTGTTACCAGAAGAGGAAGTTT
TGTTCTCTTTGTTGGTTGTTTTTCCTTAATCTTTCTCCTTTCTATCTGATTTAAGCAAAAGATAAAAAAATATCTGAAAGCTGTCTTAAAGAGAGAG
AGAGAGAGAGATAGAATCTGCATCACCCTGAGAGTAGGGAGCCAGGGGGTGCTACAAAAATAGAATTCTGTACCCCAGTAATCAACTAGTTTTCTAT
TAATGTGCTTGTCTGTTCTAAGAGTAGGATTAACACAGGGGAAGTCTTGAGAAGGAGTTTTGATTTCTTTTATATGTTTTAAAAAAAAAGCTTAAGA
AACATTGCTTTAAAAAGGAAGGAAAAAAAATACAGCAAACCATTGTTAAAGTAGAAGAGTTTTTAGGTTGAGAAATGTACTCTGCTTTGCTGAAAAG
CCACAGCTTAGGCCCTCAGCCTCACTCCCTGGCTTGCTCAGTGCCTACAGCCCTGTTACCTGATACCTGTGCTTTATCCCAGGGGTGGGCAGACCTC
TTAACCTTATAGATGGTCAGTGCGACCTCTAGTGGTCTCATGGCGTGTGGCACAACCCCCCTCCCCAGGGCTCAGCTTAATGTGCCCTCTCCCCCCA
ACAACCTGCAGGTTCACAGCGCCAGCCACACAGCGGTAGGGAGGAAATAGTGACATAATATATTCTATTTTTGTAACCTTCCTATTTTGTAGCTCTG
TWTAGAGAGATGCTGGTTTTTGCCTGAAGGCCCTGCAGCCTGCCCACATCAGGTTAAACCCACAGCTTTTGTGTGTGGTTTGTTTTGTTGTGTTTTC
GAATGGATGGGGGGATGTCCACACACGCATTCAGATGGCTGTACACAGGTTGTAGGGCTGGTAGTATGAGGTGCTTGTAAGTTTTGTTGGGTCAAGA
AGAGAGAACTCTGTTCTCGCACCACCGGGATCTGTCCTGCAAAGTTGAAGGGATCCTTTGGTGCCAGCTGGTGTTTGGAAGTAGGAACCATGATGGC
ATTACCTGGACAAGGAGATTGGGGACAACTCTTAAGTCTCACACAGGAGGCTTTTAAACACTAAAATGTCTAATTTATACTTAAGGCTACAGAAGAG
TATTTATGGGAAGGCTGCCCATGACCAGTGTGACTCAAAGCAATTGTGATCTCCCTTGATTCAAACGCACACCTCTGCCCTGCTGGAGAAGGTTTAG
GGCCATGTCTGAGAGATTGGTCTTTTATTGGGCAACGGGGGGGGGGGGGGGGTCCTTTTTAATTTAAACCACAAAGACAGAGATTTGGTCTGCTTGA
CTTTTCCCAACCCAATTGGCCCCATTGGAGAGCCATCCAAACTGAGGAAATTAGGGGACTCCAAAAGAGTTTGATTCTGGCACATTCTTGCCGCTGC
CCCCAAGTTAACAACAGTAGGTAATTTGCACACCTCTGGCTCTGTGCCTTTCTATTAGGACTTTTTGGCAAAAGGTGGAGAGCGGGAGGCTTAAGAG
GGGATGTGAGGGAAGAGGTGAAGGTGGGACCACATGGGACAGGCCACGGCTCCTCTCATGGCGCTGCTACCGATGACTCCCAGGATCCCAGGCGTTC
AGAACCAGATTCTCATTCTTTGTTATCTTTCACGTTGTTTTCGCTGCTATTGGAGGGTCAGTTTTGTTTTGTTTTGTTTTGCAATGTCAGACTGCCA
TGTTCAAGTTTTAATTTCCTCATAGAGTGTATTTACAGATGCCCTTTTTTGTACTTTTTTTTTTTAATTGTGATCTATTTTGGCTTAATGTGATTAC
CGCTGTATTCCAAAAAAAAAAAAAAAAAAAAAAAAACAGGTTCCTGTTCACAATACCTCATGTATCATCTAGCCATGCACGAGCCTGGCAGGCAGGT
GGGCGGTTGCCTCCAGGGATCTGGGACCCTGATGGCGATCGTCCTGTCATGCTGGGCCCTTCATTTTTGATCTGGGACATAGCATTCACAGCAGTTG
GGCACCTGGATTGTTCTGTTATCGATATTGTACTTGTAGCGGCCTGTGTGCATGCCACCATGCTGCTCTGGCCCGGAGGGATTTGCTCTGAGTCTCC
GGTGCATCATTTAATCTGTTAGGTTCTAGTGTTCCGTCTTGTTTTGTGTTAATTACAGCATTGTGCTAATGTAAAGACTCTGCCTTTGCGAAGCCAG
CTGCAGTGCTGTAGGCCCCCAAGTTCCCTAGCAAGCTGCCAAACCAAAACGGGCACCACCAGCTCAGCTGAGGCATCCCAGCCAGGCAGGACCCTTG
AGGGCCGCTGTGTCCATGGTGATGGGGTGAGGTTTTGGCCAAAAGGCCAAAGACTGGTGGTGGGTCCACGGAATCTGCCCTGTGACATGAAAGGCTT
TGAGGGACTCTGGCTGGTGGCCAGGTTGGCTTTTTGTATTTCTGGTTGACACACCATGGCGCTTCCCAGCACAGACATGTGACCAGCATGGTCCAGG
AAAAAAAAAAAAGACAAAAATCTAGAAATAAATTGGTAAAATCTCAGCTCACTGTTGTCTGTGTTTTCTGGGAACAGGAGTGGGGGTGATGCCACAG
CCATGTGGGGTGGCATCTCTGGCCCTGGCACCAACCTGGGATTCCCAAGGGGAAGGGTGTATGCAAGTGACAGCCAAGAGCAAAGTGGGAGCCTCTG
GCGTCAGGGAGGCCAGCGACAGCCACTGATCAGAAGCGGCTCTGGGTCCATCACTGCCAGCTACACCCTCCTGCTTATTTGCAGATGGAGATCAACA
GAACCCGCGTTCACTGCCCTTGTCTGGCGCAAATGGGTGTTTGCTCTGGGTGAATCACCGTTTTCTTAGGTGCTGCCAAGCGCCCAGAGAGCCATGC
TTAGTGTTAAGGGCCAAGACCCAAGGTGGCCTTTCACATAGTTCTGCAGACCTGAGTGGCCTGGCAAGACTCCTGTGATGTCTTCGGGTGGCAGTTA
CTCATGACTAGTATTGTTTCCAAACCCTGTGGGGACAGGTGGCTTTTAGACATCATCTTCTACAAGGTAGCTTTTGGGTCTCAGCTTGGCTGTTTTG
GTGCCTGGTGGGCCCCTGCCCTTCTGGAAAGTGGAGGGTGTAGAGGACACCTGGTCTGAGTGGTGCCACACCAATGATGAAGCAGAAAGAGATGGAC
GGAGGGAAGCCCACAGTGACCTTGCCAGGCGACCTACTCAATGAGAGTATATGGGAACACCAGAGCCAGAGGTGGCTTATGAAGACACAGTACTCAT
CTTGTACCAAATTGTCTGATGGCAGGAATCCAGAAGGTATTTTGTCAGCCAGCTCCATATAACATCCGAGCCCGGGTCCACAGTCACATGGCATCCA
GGAGAATGGGCATGGGGCCGTCAGCATTCCAGGAAATGGCCAAAGGCATTGAGGTGGAAGAGTGCTTTCCCCCTTGGTTCTTAGAAGCACTGGGGGA
GCGAGCTTATCGTGTCCAGGTAGGCTGACATGAGGAAATCCGCTGGGGTGCAGCAGCCAGGCAGCCAGTGCAGCAGAGTGGTACTAGGAATGCAGAC
ACAAATAGCAAGGGGCTAAGCATCCACTCAAAGGCTGCTGCTGCTGCTGCAGCTGCTGCTTTCCACCACTGCCTATCAAAGGTGCTCATGGAAGGGC
GCCCAACTGGAATGGGCAAGCATCACCATCAACTGCACAGGGCTCACACATGGTGCCGTCTCTTAACGGTGCAATTGGCTTGTCTCGTCATCCAGTG
GTCTGTGATTCCCAGCTGTGGCTGATAGCCAAGCCTCTTTTCTTGGTTTTAGCAGTGAAACCCCAGATACCACTGGCCACTCCAGGCTCCAACCCTC
AAGTTTAACTGGTAACACTGGAGGCTAGATCCTCTGCTGGGTGTAGAGATACTCCTGTTCTAAGGGTGATGGTAAAATAGCTTTTGAGGGCCCCTCA
GAGATAAGGACTCAGGGAGCCAGCACACCCAACTGTCTCCTCTCAGAAGCTCAATGGCTGGTTCTCTCTCTGCCTCTACCAGCTCCATGGTCCCCTG
CCCTGTGTCCTGGCCAGGCTCTCCTTTTGCAGGCGGCCTCCATCCTGCTGAGCCATCCTACCTCTCTCAGCCAAGCCAAGCTCTTGAACCTGCCTGG
CCTCTCTGAGCCTCAGCTCCCTGAACTGGAAAGATTGCCCCTGGCTCCCCATTCTGTGTTGCCCCACTGCCTGGACCTGCTCCAGCCTTCCTGTTTG
TCCTACAGGACTCACAGTGAATGTGGCCCCTGTGGAGCATGTGGAGCATGACATTGCACAGGTGACTCTGTGTAGCCTCTCCCCTCCTGACTCCACA
TCCTAGAACCAAGTGTGAGGAAGTTAGCCTGTTGCCCATGGGTCAGCTCAACTGCTGCACTTTGTCCCAGAAAGCAGGCCAACTTCACCAACCCTAC
AGAGGATCTGGGCTGCTGGGGCCCACAAAATGCCAGCAGTGGGAGCTGAGGACTCCAAGGGGTGGGACACAGGCAGAAGAGTAGGACTGTGTGTTCT
CCCTGCAGTGCCCCAAAGCCCTGGCCACAACATCCCAGGAGACAGTCAATGCGGGTTGGGCTGTGTGTTTGGTGGCAAGGCTATGCAGGCCACATAA
GGCCAGTGCTGGGAGCTGGCACTGCCAAGTGTGCTTGCCAAGCAAACCTGTACTTGAGCTGATAAAGCAGTCTGTTGAGTGTGTGGGGTGGGGTGTA
TAGGTAACATGAGGCGGGCATGTTGTATACAGGGCCCCACGTGGGCACAGCACAGTGCTGGTGGGATGCAGTACCCCTAGTTCTGAGCTGATCTGGC
CTCTCTTACTCAGGGATGTCATCCCAGGAATGTATTTGATCAAACAGTTGTGCAAGAGGACAAAGCAAGCACAGGANNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNUNNNNNNNNNNNNNNNNNNNNNNNNNNNNNNNNNNNNNNNNNNNNNNNNNNNNNNACTAAGTAAC
TGCTAGATCCTTGGACTTCCATTCACAGCTGCTACTGAACCATTGTTGGGAATTGGACTGTAGACTGTAAGTCATCAATAAATTCCTTTACTATATA
GAGCCTACCCATAAGTTCTGTGACTCTAGAGAACCCTGACTAATACAACATGCATACACATAAAATTAAAATGTGCCAGGCAGTGGTAGCACATGCC
TTTAATCCCAACACTTGGGAGGAAGAGGCAGGTGGATTTCTGAGTTTGAGGCCAGCCTGGTCTACAGAGTGAGTTCCAGGACAGCCAGGGCTACACA
GAGAAACCCTGTCTCGAAAAACCAAAAAAATAAAAATTAAAAAAATAAGTAGGTAAATTAAATTAAATTAAAATGTTTAAAAAGAAGAAATCTTGGC
TGAATATTCTACAGTTTTTTAAGAGTTTGTCAAACATTTTGATGTTACAATGGCCAATGATGACAGTAGCCCCTCACATAAATACATAGTACACAGG
GCAAAAGTGTGCACAGGGCCAGTTCAGAAATTGCTGGAAAATTCTGTCCTGACCCTAAGACAAGTTTCACTGAACAATTTTTTTATGAAAATGGTCG
GGCTGGAGAGATGGTTAAGAGCACTGACTACTCTTCCGAAGGTCCTGAGTTCAGGTCCCAGCAACCACATGGTGGCTCACAACTATCCATATTGAGA
CCTGACGCCCCCTCCTGGTGTGTCTGAAGATAGCTACGGTGTACTTACATATAATAAATAAATAAATCATCAAAAAAAAAGAAAATGGTCAATTTTT
AAAATCAGACATTCTAATATAATACAAACCATAGCTATACTAATTTGACACCTGTTGAGGCCTGAGAGCACAACACATTAAAGACTTGGCTTGCCAT
AAGGAACCCGTCATGCCTCTGTGAGAGCAGAGAGCTGTGTACAAGATGGATGGACGTTGTACTTCTTACAATGCTGGTCTGGGTTGAGGGGGTTTCA
TTCGGGTGCTGTGTGTTGGTGCAGATGGAATGACCTCGTGCAGTACAGAGCACAGAACCGAGACTGCATTGAAGTCTTGGGATGCAATATAGGTGAG
CGTCTAGAAGCACCCTTAGATCTGATTATGCATTTGATGTACTTCATTTTTAGACAGGACCTCACTATGTAGCTTTGGGTGGCCTGGCTGAGTAGAC
CAAGCTGGCCTCAAATTCCAGAGGATGCATCTGCCTCTGCCTTCTGGGTGCTTGGAATCAAGGTATGCGTGCCACCAGGCCCAGTAAATTTAATTTT
TTCAAATTAATTCTTTTCTCATGTTTAGAAATCTTTTCCTGGTTTTTGTTGTTGTTTGTTTGTTTGTTTGTGACTCCTACATTGGGTTTTTGTGACT
CCCATACAGAATTGGGATCCACAGTTTTTAAGAAACTGGGCACTAAATTACCCCTGCCTTATGCAAAGCCCACAAGGACAATCTGGAGAGACCGCTT
ATGTGAACAGCAGGAGCTGACTCTGTTCAGGGAGCTAAAGGGTAAAACTGTGTATGCTGGACCACTCCATTTCTGGGGGTTCCTGGCAAGCCTTAAT
GTTACTCATCTGGACCTCCCTCTATTTTCAGGCTGTCTTGGGTCTTTACGTTGTAGCTTCTCATGGCCTCCTCCTCAGGGTCCACTACCACCCACCT
TAAACAATGCCAGTTCCAGCCTTTCAAGCTGAAATCAAGTGTAAAAGGCCCAGCCATGCTTGAGCAGGCCTTCCCTGCACAAGTTCAACCACAAGCA
AACTTCTGAGTGAACTGGGTGGCTAGGACCCTTCCAGAGCAGACTTGGCAGCAGGCCTCTGAGCTTGAGAGCAGGTTCCAAAACTCTTGGTTTCAAG
GGTCCGCACCCACTGGCAACAGTAAGGAACCTTCTGGGAGAATGGGCAGCACTTTCAAGTCTTCAATCCTGCAGACACTTGGTCTTTAGGCACTGAT
GCAACCTCTCCAATAGAGGACTAAAGGACCCACCATCCATGAAGCATGGGGACAGGCACTCCAGCCCTGCAACCCAGGAATGGCCAGGGTCTTATGG
AAAGTGCCTCTGGGAAGCTGACCACAGAAAGGCAAGGAGTGACTTCCCTTCTTAGAATATTAACCTGAGGTCACCCTAACTTCTAGGAAATGTGAGC
AAAGCCCTATTCACCCCAGATGGGGCTCCAGTGACAGACCAAAGTATACTTCTGCCACAGCCCAATTTGGTGAACCAATGAGTTTCTAAGTGGTTAG
TTGGTTATAGGAGTGTGGGAGACACCCCAGCTACAGATAATGATCTTGTGGGAGTTAAAAAAAAAGGGTCCCCACTTCTAGTATACTCCTGAATCCC
CTAAGAGCTCTGCTGCTGGGGACAACCAGGTGAAGTAGTGGCTGGAATCCCAGGGTCCTCCCTGCCTTCTCCCTGTCTGTCACCCCCTTACCATGTG
GTTATCTCTGTAACTAGTTGCCACAGCAACCTACCCTACATCATCATTAAGATGGTGGTAGAGTGAACTGAACTACAACACAGAGCCACAGGCTGCT
GACCTTTGAGACAGGTTCCTGGTTGTCAGGGCTCTGGGGTCCCATGCCTCATTTTCTCTGTCAGCCCTGTGACTTCAGTTTCCTGAGATACACAGCA
ATTTCTAGCTCTGCCTCATAAAGCTGGGGAGCCACTAAGGTCAGTGGGGCAATCTTTCTAGTCAGCACACTTTGGTCATCTCCAGGTGGAACCTCGG
TGAACATGGGAGTCTCCCTCCCAGGAAGTCTCCCTGCCACCTCCTCAGGTAACTGAGTGCTCCCCTACCTCTGGGCTCCCAGAAGGTGGCAGCCATT
ATCAGAGAGCCCACACGGGGTCTGAAGCATCTGACAGCCCTTCAACACTGCTCCTTCTCTATAAATCCATGCTTTTCCTAGACCTGCTAGATTCTGA
CATGCCATAGGCTCAAATGAGCAAGAAACTAGGCTGGAGCAGCAGGCACAACTCTCACCCCACTATGCTTTGTCCCGCCTGGAATTGGCCCTTTACT
GTGACCTCCTGCAGTCTCTCCACCAGCCCACGTTGCTGACTACAGTCTGGTACATGTAGACTTAGTGGTATGGGGGAAGTCAGGGCTTTAGCGCAGA
ACTGTGGCCCCTTCTTCCAGTTTCCTCATCTCACTACCAGGCAGAGGAAGAGGTTTGGGGGTAAATAGGGAAAATTGGGAGGGAGTGAGCCAGAGTT
TCTGGGTGTTTGGTCTCAACACAACTATCCTACAAACATCACCATCAGCAGGGCTGGGGGATGGAGAGGTGGTTAAGGGTGCTTGATGATGCTCTTG
AAGAGGACCCTGCTTCAGTCCCTGACCACCATATGACAGCTCACAAGCACATGGAACTCCAGTTNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNAGGCAGGCGAATTTCTGAGTTCAAGGCCAGCCTGGTCTACAGATTGAGTTCCAGGACAGCTGGGCTAC
ACAGAGAAACCCTGTCTCGAAAAACCAAAAACAAAACAAAAACAAAACAAAACTCCTCACTAACACTAGCTTTATACAGAACTTCAATCTGTATTTA
GCTCCAGGCTACAATTGGTCCTGCAGTCACAGTTAGGGATTACATAGTGTCTCTGCAGGCCCAAAGGCCCTTCTGGATCACAGTTTAGACAGACTGC
AGGACCATTCTGCCAGGCCCTCTCTCTGTTTAGTTACTTTTCTGTGTTGACAAAAAAAAAAAAAAAAAAAAAAAAAAAAAGGCAATTTGCGGGATGA
AGGAGTTTATTCTGTCCAGTTTATAGGTGGCATTCATTATGGTGGGAGGTTGACAGTGGAGCCTGAGGCAGATGCTCATATCGCCTTTCCACGGCCG
AGTCACAGGAAGCCAGAACAGGGTGGGATGAGTGATAGAAAACAGACCGGTAGTGGGGCACATAGCTCACAGGACATGGTCTGGAGAAGGCAGGGGA
TTGACCAGCAGCTCAGTGATTATCAAGGACACAGGTTCCACTGGTACCTGACTCTGCCACCTTCAGCGACAGCTTGGCCACAGCTCCCACAGTTGTC
TATGGCAGTCCAAGTAACACCTTCATGGTACAGCATCCAAAAGCAGAGCATTCTTCTGACGACCTATGCCCTCCCCTTTCACAGCAGGTGTGACTCA
GTGGTGGGAGGAGGCGGCATGCCCACGTGGCAGAATGGTCCTGTAGTCTGTCTAAACTGTGATCCAGAAGGCCTTTTGGGGTAACCAAAGCATTCTG
GGGACTTGATGGGGACTTTGAGAAAGGGTCAAGTGGGCAGAGGTCAAGGCTTGTACAGGAACATGGAAAGTTATCAGGCAAGCGGCTGTGGTGATGC
TGATGGGTGGGAGATCTGACACCTGATGTACACAAGGTGAGGGGATAGGAGGTAGAGATGAAGGGAGAGAGAGAGGGAAGAGACAAGAGAGGAACTA
GCAACTGATTGATACCTGTAGTGATGTGACAGGCACAGTAGGCCCATATCTGTGTCCTACATCTTGATTGCACCATCTTGGCACTGAATGAGGACTG
CTGCAGCTGTCAGTAGAAATGATCCCAGACAGACTACTGCTTTAAAATACACTATTTATTCTTTTTTCTGAATATAATAGGAAAGTAAAATATGGTG
ATAACAGCAGCTCAGTTCTCACACCGAAGAGTCCCTGATCCTGGGCACCTGAGAGAGTGGTGCTGAATACCTGAGAGGTAGCCGCCAGGCCCAGGAG
GCCAGGTGCATGGTGCCCCAAGCCTCAGACTCAAAACATATTTACAAATCCTTCGGACATACAGCTAGGCTCTATTCTGACTAGATTATAACTGAAG
ATAACCCATTTATTTTAACCTGCATTCTGCCAGCTGGCTGGTTACCTGTGCTCAGGTACCATGTGTQCATCTCCTCACATCCTCCCTGGTAGATCTC
CCACCTTGCTCTATCCCAGAATGCTTTCTCTCTCCCAGATGTTCCGCCTATTTCCTGCCTAAGCCATAGGCCATAGGCTTTTTGCCACAGGTGAGGC
ATCCATCAACACAAAGATATTCTTCTCTACGAAATGCCTGAGAGCACTGTGCCACTCCTGCCAGCTCGTCTTATATCTGCCTGTAAATACCATGCCA
GACACACAAGCTGTTGCTAAAGGAAGTRCAGTGTCACCAGGCAAGCCCTACACATTCACAGTGGCGGCCACCAATTAGTCCTGCTTTGGTTTCCAAT
AGTTCCCCTAATGCCACTTTTTGTTTGTTTGGTTTTGAATTCACTTTGTAGACTAGGCTGACCTCAAACTGAGAGATCTGCCTACTATCTCTTGAGC
CTGCTTCAGCTTCCTCCCTTGTCTCGTCACCCTTGTCTTCTCAGCTGGGCATCCAGAGGCAAACCAAGTCCAGTGGAGCCTGAGTCCTGTGCAGATA
GCAGGAATCAGGTCCTCCAGTGGTGTTGGCAATGACAGCGCATGTTGGAGGCAGAGGCACCATGTGGTACTTCAGGGCATGGACCTGGGGTGGGGGC
CAGCTCTGCCACCAGGCCCTTAGACTAGATGGGTCTATTTTATCACTTCTAAGCCATAAGGTATCCTCCACCTCTGGCGCTGCCAGAGGGATGGAGT
GAACAAGGAGGCCCAAGCATGGCCAGCAGAGGCTCAGCAGGCAGCAAAGATCCTGTCACGGCTTTCCCATGACCCATTTCACATCGGCCTGCCTCTG
TGAGTCCCACCCTCAGGGTAACCCTACCCAGGGCCACTCACTCTGGCCAGTGACAAGCGGTCTTTTGAGTGTGTTGCCCCTGTGCAGAAGGCACGGT
GGTCAGGTAAGCCACCTAGGCAGAGACTCAGGGAAGAGCCCAAGCTGGTCCTCCCACAGAACAATGGATTCCTCTCAGGCTTGCCTTTAATCACTGT
GACACTTAATGTGCAGACATCTGCTCTTCTGTGGGTCCTCAAAACCCAAGTCCAGAGCCTCCTGGAGTCACCTTGAAGTCTGGCTGCACGTTGAGCA
GTGAGCACAGCTGAAAGTACCCAAGAGCCCATGCCTTACACCTCCTGCACTTCTGCTCAGCTTTCCCTATCCCAGCACAAGCCCAAAGGCTATGTCG
CCTTAACGTCCCCAACCCACTTTCAGTCATTCTACAAGGGCCAAGGAAACAAGTTCCTCTGGGTCTATATCTGGGCACACATTGCCCAACACAACCA
TCACATGAACCCTAGGGCCCACGCCAACCTGGCCTTCAAAGCCCCCTCAGGCACACCCCATAGAC
MOUSE SEQUENCE—mRNA
GAGGGCTGTCGGCGCAGTAGCAGAGAGCTACAGACTCCGCGCGCTCCGGAGACCGGCAGTACAGCGCGAGGCAGCGCGCGTCAGCAGCCGCCACCGG
AGCCCAACCGAGACCACAGCCCTCCCCAGACGGCCGCGCCATGGAACACCAGCTCCTGTGCTGCGAAGTGGAGACCATCCGCCGCGCGTACCCTGAC
ACCAATCTCCTCAACGACCGGGTGCTGCGAGCCATGCTCAAGACGGAGGAGACCTGTGCGCCCTCCGTATCTTACTTCAAGTGCGTGCAGAAGGAGA
TTGTGCCATCCATGCGGAAAATCGTGGCCACCTGGATGCTGGAGGTCTGTGAGGAGCAGAAGTGCGAAGAGGAGGTCTTCCCGCTGGCCATGAACTA
CCTGGACCGCTTCCTGTCCCTGGAGCCCCTGAAGAAGAGCCGCCTGCAGCTGCTGGGGGCCACCTGCATGTTCGTGGCCTCTAAGATGAAGGAGACC
ATTCCCTTGACTGCCGAGAAGTTGTGCATCTACACTGACAACTCTATCCGGCCCGAGGAGCTGCTGCAAATGGAACTGCTTCTGGTGAACAAGCTCA
AGTGGAACCTGGCCGCCATGACTCCCCACGATTTCATCGAACACTTCCTCTCCAAAATGCCAGAGGCGGATGAGAACAAGCAGACCATCCGCAAGCA
TGCACAGACCTTTGTGGCCCTCTGTGCCACAGATGTGAAGTTCATTTCCAACCCACCCTCCATGGTAGCTGCTGGGAGCGTGGTGGCTGCGATGCAA
GGCCTGAACCTGGGCAGCCCCAACAACTTCCTCTCCTGCTACCGCACAACGCACTTTCTTTCCAGAGTCATCAAGTGTGACCCGGACTGCCTCCGTG
CCTGCCAGGAACAGATTGAAGCCCTTCTGGAGTCAAGCCTGCGCCAGGCCCAGCAGAACGTCGACCCCAAGGCCACTGAGGAGGAGGGGGAAGTGGA
GGAAGAGGCTGGTCTGGCCTGCACGCCCACCGACGTGCCAGATGTGGACATCTGAGGGCCACCGGGCAGGCGGGAGCCACCAAGTAGTGGCACCCGC
AAAGAGGAAGTACCCAGCCCGGGTGCTCCTGACGACGTCCCCCTTGGGGACATGTTGTTACCAGAAGAGGAAGTTTTGTTCTCTTTGTTGGTTGTTT
TTCCTTAATCTTTCTCCTTTCTATCTGATTTAAGCAAAAGAGAAAAAAATATCTGAAAGCTGTCTTAAAGAGAGAGAGAGAGAGAGATAGAATCTGC
ATCACCCTGAGAGTAGGGAGCCAGGCGGTGCTACAAAAATAGAATTCTGTTCCCCAGTAATCAACTAGTTTTCTATTAATGTGCTTGTCTGTTCTAA
GAGTAGGATTAACACAGGGGAAGTCTTGAGAAGGAGTTTTGATTCTTTTATATGTTTTAAAAAAAAAAGCTTAAGAAACATTGCTTTAAAAAGGAAG
GAAAAAAATACAGCAAACCATTGTTAAAGTAGAAGAGTTTTTAGGTIGAGAAATGTACTCTGCTTTGCTGAAAAGCCACAGCTTTAGGCCCTCAGCC
TCACTCCCTGGCTTGCTCAGTGCCTACAGCCCTGTTACCTGATACCTGTGCTTTATCCCAGGGGTGGGCAGACCTCTTAACCTTATAGATGGTCAGT
GCGACCTCTAGTGGTCTCATGGCGTGTGGCACAACCCCCCTCCCCAGGGCTCAGCTTAATGTGCCCTCTCCCCCCAACAACCTGCAGGTTCACAGCG
CCAGCCACACAGCGGTAGCGATGAAATAGTGACATAATATATTCTATTTTTGTAACCTTCCTATTTTGTAGCTCTGTTTAGAGAGATGCTGGTTTTT
GCCTGAAGGCCCTGCAGCCTGCCCACATCAGGTTAAACCCACAGCTTTTGTGTGTGGTTTGTTTGTTGTGTTTTCTTTCTCTATGTTCCAAAACCAT
TCCATTTCAAAGCACTTTTGGTCAGCTAGCTGGAGGCAGTGTTGCTGGTGTGTGTTGGGGGGAGGGGTTCTAATGGAATGGATGGGGTTGTCCACAC
ACGCATTCATGGCTGTACAACAGGTTGTAGGGCTGGTAGTATGAGGTGCTTGGGAAGTTTTGTTGGGTCAAGAAGAGAGAAAACTCTGTTCTCGCAC
CACCGGGATCTGTCCTGCAAAGTTGAAGGGATCCTTTGGTGCCAGCTGGTGTTTGGAAGTAGGAACCATGATGGCATTACCTGGACAAGGAGATTGG
GGACAACTCTTTAAGTCTCACACAGGAGGCTTTTAAACACTAAAATGTCTAATTTATACTTAAGGCTACAGAAGAGTATTTATGGGAAGGCTGCCCA
TGACCAGTGTGACTCAAAGCAATGTGATCTCCCTTGATTCAAACGCACACCTCTGCCCTGCTGGAGAAGGTTTAGGGCCATGTCTGAGAGATTGGTC
TTTTATTGGGCAACGGGGGGGGGGGGGGGGGGGTCCTTAAAAAAAAAAACCACAAAGACAGAGATTTGGTCTGCTTGACTTTTCCCAACCCAACCCA
ATTGGCCCCATTGGAGAGCCATCCAAACTGAGGAAAATTAGGGGACTCCAAAAGAGTTTGATTCTGGCACATTCTTGCCGCTGCCCCCAACTTAACA
ACAGTAGGTAATTTGCACACCTCTGCCTCTGTGCCTTTCTATTAGGACTTTTTGGCAAAAGGTGGAGAGCGGGAGGCTTAAGAGGGGATGTGAGGGA
AGAGGTGAAGGTGGGACCACATGGGACAGGCCACGGCTCCTCTCATGGCGCTGCTACCGATGACTCCCAGGATCCCAGGCGTTCAGAACCAGATTCT
CATTGCTTTGTATCTTTCACGTTGTTTTCGCTGCTTTGGAGGGTCAGTTTTGTTTTGTTTTGTTTTACAATGTCAGACTGCCATGTTCAAGTTTTTA
ATTTCCTCATAGAGTGTATTTACAGATGCCCTTTTTTGTACTTTTTTTTTTTAATTGTGATCTATTTTGGCTTAATGTGATTACCGCTGTATTCCAA
AAAAAAAAAAAAAAAAAAAAAGAGGTTCCCCTGTTCACAATACCTCATGTATCATCTAGCCATGCAGAGCCTGGCAGGCAGGTGGGCGGTCTGCCTC
CAGGGATCCTGGGACCCTGATGGCGATCGTCCTGTCATGCTGGGCCCTTCATTTGATCTGGGACATAGCATCACAGCGGTTCAGTCACCTGGATTGT
TCTGTTATCGATATTGTTACTTGTAGCGGCCTGTTGTGCATGCCACCATGCTGCTGCCCGGAGGGATTTGCTCTGAGTCTCCGTTGCATCATTTTTT
TCTGTTAGGTTCTAGTGTTCCGTCTGTTTTTGTGTTAATTACAGCATTGTGCTAATGTAAAGACTCTGCCTTTGCGTTCGCAGCTGCAGTGCTGTAG
GCCCCCAAGTTCCCTAGCAAGCTGCCAAACCAAAACGGGCACCACCAGCTCAGCTGAGGCATCCCAGCCAGGCACCCACCCTTGAGTCCGCTGTGTC
CATGGTGATGGGGTGAGGTTTTGGCCAAAAGGCCATAGACTGGTGGTGGGTCCACGGAATCTGCCCTGTGACATGAAAGGCTTTGAGGACTCTGGCT
GGTGGCCAGGTTGGCTTTTTGTATTTCTGGTTGACACACCATGGCGCTTCCCAGCACAGACATGTGACCAGCATGGTCCAGGAAAAAAAAAAGACAA
AAAATCTAGAAAATAAAATTGGTAAAATCTCAAAAAAAAAAAAAAAAAAAA
MOUSE SEQUENCE—CODING
ATGGAACACCAGCTCCTGTGCTGCGAAGTGGAGACCATCCGCCGCGCGTACCCTGACACCAATCTCCTCAACGACCGGGTGCTGCGAGCCATGCTCA
AGACGGAGGAGACCTGTGCGCCCTCCGTATCTTACTTCAAGTGCGTGCAGAAGGAGATTGTGCCATCCATGCGGAAAATCGTGGCCACCTGGATGCT
GGAGGTCTGTGAGGAGCAGAAGTGCGAAGAGGAGGTCTTCCCGCTGGCCATGAACTACCTGGACCGCTTCCTGTCCCTGGAGCCCCTGAAGAAGAGC
CGCCTGCAGCTGCTGGGGGCCACCTGCATGTTCGTGGCCTCTTGATGTGGAGACCATTCCCTTGACTGCCGAGTGTTGTGCATCTACTACACTGACA
ACTCTATCCGGCCCGAGGAGCTGCTGCAAATGGAACTGCTTCTGGTGAACAAGCTCAAGTGGAACCTGGCCGCCATGACTCCCCACGATTTCATCGA
ACACTTCCTCTCCAAAATGCCAGAGGCGGATGAGAACAAGCAGACCATCCGCAAGCATGCACAGACCTTTGTGGCCCTCTGTGCCACAGATGTGAAG
TTCATTTCCAACCCACCCTCCATGGTAGCTGCTGGGAGCGTGGTGGCTGCGATGCAAGGCCTGAACCTGGGCAGCCCCAACAACTTCCTCTCCTGCT
ACCGCACAACGCACTTTCTTTCCAGAGTCATCAAGTGTGACCCGGACTGCCTCCGTGCCTGCCAGGACAGATTGAAGCCCCTTCTGGAGTCAAGCCT
GCGCCAGGCCCAGCAGAACGTCGACCCCAAGGCCACTGAGGAGGAGGGGGGGTGTGGATTGACCCTGGTCTGTGCCTGCACGCCCACCGACGTGCGA
GATGTGGACATCTGA
HUMAN SEQUENCE—GENOMIC
CATGCCTGTAATCCCAGCACTTTGGGAGGCCAAGGCGGGCAGATCACCTGAGGTCAGGAGTTCGAGACCAGCCTGGCCAACATGGTGAAACCCGTCT
CTACTAAAAATATTTTTTGAAAAAAAAAAAAAAAGAAAGAAAAGAAAAATAGCTGGGCATGGTGGCGCATGCCTCTAATCCCAACTACTTGGGAGGC
TGAGGCAGGAGAATCGTTTGAACCCAGGAGGTTGAGTTTGCAGTGAGCTGAGACCGCGCCACTGCACTCCAGCCTGGGAGACAGAGCAAGCCTCTGT
CTCAACAAAAAAAAAAAAAAAAAAAAAAAAAAAAGGGGAAAAATCACTCCAATCATCCCAAGTCTCAGCTCCTACTCTTTTGCTATATGTCCTTCAA
ACCAGCTATGTATTTATTTTGCTGAATTTATGCTGGTTTTATCTTTTGTATCTTGCTTTTTTTCATTTAATGTTTTTCCATCCAGTGAACACACCCT
AATTTGCCATTCCCCTCTTCTTTGAACATGAGATTGTCTCTAGGGTACTCTTGCAAATAGGATAATTGTCAATGATGTAACGGGTCTCTATGCAACT
TGCAAAAGTGATCAGTCTTGGTGTCCTCCTCATCCTAGTGTTTGGTTCCAAGCAACAGAAACCAATGCTGGCAGATTTCAGCAGAAAAGGAAAATCA
GATGCAGAAGATAGGCAGGAACAAGGGAGGCAAGACAGCACGAATCACAATGGAAGCATGCTGCAGATCTTCTCTGATGAGAAGCCAGGGCTACCCC
CTGCTCATAAACTTGAATGCCTCACCCAGTTCTACACTAGAAAACATTTCCTACCTTTCCTGATTCTTCCCACCACCAGCTCCCAATTCAAAATCTT
GATCCAAAAAGATGAAATGTATAAATAATACTATTTATAATGACATCAAAATATCAAGTACTTAAGAAGAAATCTAATAAAAGATGCACAACACCCA
ACACACACACACACACTCACACACACACACCTGCAATTAAATTAAGGACAATCTAAATAAACAGAGGTCTATATCATGCCCATGAATTGAATGACTC
TGTTAGTAAGTGTCAATTCCCTCAAAATTGGATCTATAGATCCAATACAATCCCAATCAAAATTCCAGCATTTGTGTGTGTGCACATGGGTAAGTTG
ATTCTGAAATGTTTATGGAGCCCCAAGGGCCAGGAGTAGCCAGGGCAACCATAAAAGAGAAGTGGGTTGGAGGATTTAGCCCGCCAGATATCAAGAA
TTCGTAGCGTATAGCTTCCATGATGCTTCTGGGGTAAGGACTGAAAAAGAGACCAGTGGAACGGGATAGAAAGTCCAGAAACAAGAGATTTGTGTTA
TATATGTATCTTATAACATCATGCTGTCAAGTTTATTTCAAAAATAAXTTTTACATAAAATCTTAGCAGTGTTATTCATAATAGACAAAAAGTGGGA
AAATTCTATTTTTTTAAAGTACAGAAACTTTATTCCTCAAAGGGGAAATAGGTTAATAATAATAAGAAGAAGAATTAAGGCCAGGTGTGGTGGCTCA
TGCCTATAATCCCAGCACTTTGGGAGGCCAAGGCCAGAGGATTACTTGAGCCCAGGAGTTTGAGACCAGCCTGGGCAACATAGTGAGACCCCATGTT
TACAAAAATTAAAAAATTAGCTGGGTGACGTGACAAGTGCCTATAGTCCCAGCTACTTGGGAGGCGAAGGCAGGAGGATTGCTTGAGACCTGGAGGT
CAAGGCTACAGTAAGGTATGATCGTGCCACTGCACTCCAGCATGGGCAACAGAGCAAGACCCTGTATCTAGGGGGAAAAAATGATAAGCCTTTGTGA
AAGAGAAATGAAAACAGCATCCACACATAAACTTGTACCTCTGTGTTCATCGGCAGCATTGTCCACAGTAACCAAAGGTAGAAACCATCCTAAGGCC
CATTGACTGATGAATGGATAAGCAAAATGTGGTCTATCAATACGTTTGATAGTATTCAACCATAAAGAGGAATGAACCATGGACACATGCCACATGT
GGGCCTTGAAAACGTGATGCTAAGTGGAAAAAGCCAGATGCAAGAGGCTGCATAGCATATGATTCCATCTGTTGGAAATGTCCAGAAAAGGCAGATC
CACAGAGAAAGATTAGTGGTTGCCAGGGGCTGGGGAAAAGGGGGAAGAAAAGGGTGAATAACTACAAATGGGTACAGGGTTTCTTTGGCGGATGATG
AAAATGTTCTCAGATGGTGATGGTGGCTGTCCAACATAACAAAATCCATCGAACTATTTACTCTAAACGAGTGAACTTTACGGTATGTAAGTTATTT
TTCAATAAAGTGGTTTCAAAAAAATAAAAAGGTGAACCCTACCATTTCTAGGTGCTTTGGAGTTTACCGTTTTTCTCCTTGCATTCTGGAGTCAGAG
TGAGAAGGCATCTCAGCTCTACCACAGCTGAATGATGTGTTGGGCTTCGGTTTCCTCATCTGTAAAATGGGGATAAAGGGCCTCTCCTTGGAGCTGC
CATGAAGATTGAATTAGATGCCCTCTGTGAAGATGCAGGGCAGGTGGAGAGCACAGTAAGCGTCTCGGGGGGCCACGCCATCACCGCCAGCAGGGCT
CACCAGTATCTTTGCCCAGAGAATAGCCGCGGTCTAGGGAGCAGCCAGGGTCTAGGGAGAAGGGCTGCTTCCCTGTGGAGGCTCCTAACTTTCCCAT
CTCCTCCCTGTTTCTCCCCTGCCCACCCCTTTACTCCTGGAAGTGTGACTCGCTGCCTCCTCCCGTGACTCCCTACATTGCATCTCCCCAGTTACTG
TCGTTATCTCTCATCTCTTCTTTCCAAACAGCCTCTGTCTAGCACCTTGGAGCTCGCATGGGAATTCATGGGTGGGTGGATTCCAAGCTTGGCTTCA
TCAGATGACAACTAAAAGGCAGAGGCCTGGGGGGGCAAGGGAGCTCCAGGATCTGTCTCCCTTTGGAGAAGCCCAGGTTCACCTTCCCAGCAAGCTG
CTCCGGCCCCACGCCCCTGGCCCTCAATAACCTTCAGTGGCTCCCTTCTTGCAGCTATAATTCCCCTCTTGGGATGCTACAAAAGGGATTCCAGAGA
TTTTTTTTTCATACTCATAAGGGATGGAGTAAACGATATATGTGCCCAGGACAAGGCAGCTAATGGGAGAGGCCTCAGGCTTCCTTTGCCTTTGCCT
GGAAGTATACCAACCCGCAGCCCGGAGGGCCAGCAGAGAACCAATGCTCAGAGCCTGGGGGCCCAAGGAATGCTTTCCAGGCTGGCGGTCCCAGGAG
CCCCCTCTCCCTTCCCTATAGCAAGATACTAAGAGCAGAGGCTGGCCTGGCAGGCCGTGTCGAGGCCCTGAGCTCACATGGCCTTGGCAGTTTCTTG
ACAGTCGTGTCTTTTTGTTTCCAGCAGTGACACTCACTGACCCACACCTTTGGCCAGCGTCCCTTGTGACTGCCCCCTCCCGCTTGAGCAGAATGGC
TCCTATTACACAATGGGTGCCAGGAACCTGGCCAGTCCATCCAGAGACAGGGCAGAGCCCTGGGCTACCGACCCTCTGGGGCCAGCAGAAACCCTCC
CTATGGCTCTGCCTCTCATCCTGGCATTTGACACTTGTGCCCTTAATTAAAGTGCTTCTTTTTAAATCAACATTTCTACTCCTTTTGCAGAGTAAGA
GAAAGAAGAGTTAGGGGAGAAGAAAGAAGGAAGGATTCATGGGGTTATTTTTTTTTTTTCATGTAAAAGTTTGTTCCGTGATTTAAATCTCATGTCC
ACAAACAGCTCAGGAGAAGTCAGCATAGAGAAGCAAGTGACAGATTATAAAGTCATTTGTAAAAATCCTCTTTCTGCATATAAACTGAAAATGTGAA
TTAATTCCACATACTGCGTGTGGCACATGGCCTTCATCCTGCATAAGAACAAATACATGAATGAATAAATCGAGCAGGCCTTGACACTCACCAGGCC
CTGTGCTAAATGCTTTCTACATATTACCTTAATTACTCCTCACCGTGGCCTTGCGAAGATAACGATCACAGCCCGTTTTACAGATGGTGTCAGAGAT
TTGTCCAAGGCCACACAACTAGTGGCAGATGTAGGTCTGTATGTGACATTCTTGTTCAGTTTCATAAATGTGGCTGCCACCTGCCTGAAATTTTGAG
GGAAAAAAAAGTGGTTTGGCTTTGCATGAATTCTTGAAAAAGTAGATTTTTATCTTTACCAGCTTCTAAGCAAGCTGGATTTATTCTGACAGTGTCC
ATGAAATGGCTACAGACCATCACCGTTGCCATGTTGTGAACCTGAACAAGGCACCTGGTTTCTCTGTGCCTCGGTTTCCTTACCATTTTTTCGCATT
TCCCTGAGATTAAGTGTGTGAAACTATAAGTTGGCTGGAGGAAACACCTCATAGAAGTGAGAAGATGAGGATGGGTGGTGAGTGAAGGCTTTTTCTC
CAACCCTACAGGAGATGGACCTCAAAGCAAATGGCCATGCTTCCCTTAGACTGGGTGAACTGGTGCTGCTCAGGGCTAGAAAGTGTGTCTAGAAGCA
TGGGGACCAATGCAGCCCCCTCCACCCTAGGGCACATAACCTGTCTCTCTCCCATAGGATAGCAGAGACAGCTCTCTAAGTCAGGATTCAGCGAGAT
GGTCCAGGAGGGGCCTAAGGATTCCTCTGACTTATCCAAGCCTGGGGAGGCAACACAACCTAGCCTGTGCTCAGGGGGGCCCCAATCATGCCCTGAA
CCTCATGAGCCCAGGCAGGCATCCTGTTGGTTAAGGTCGGCCTCAGGGTCCAGGGATGCACTAGATTCTTTATCTTAAAGTGACCCTCAAGAAAAAA
AAATAAATGTAGCTATAGGCAGTTTCTGGACGTTTTCCTATTTGTTAAGCACCTTCTTGCTCCGAGCTTTGTTCCAGCCACAGTGCACTGGACGCTT
CGGAAGGCTGCAGCTGCTATTTGGTGCTTTACCACCGCTGAGAAGGTGGAATGGTCACGGCCCGGTTCTGTCACTTTCTTCAGCCGGACAGTCGCCT
TATTACGGATTCCAGTAGGGCCGAGCACACTCTTCCTGCCCGTCTTTACAGATGAACATGCTATCGCTCCAGCAGTACAACCCCGCCTTATTACGTA
AAAAGCAGCCCCTATCTACCCGCAGGGAAGGAGTATGTTCGGCACCACAGCTGGACTGGTGCTCGAGTTTTTGCGTCCTGGGAGGTCCGCATGCGCT
CCGGAACCGTAATGCGCGCTTTTTTCTAAGCCTTACGGTAAACGCGGACGCAGGGCAACCACGTGGCGGTGGAACCGAGGCCGGCGGGAATGCGCGG
AATGCGCGGCGCGGCCTCGCGCGGTTCCCGAGCCACGGCCCAGGGTCCGGCGGCGCGCGCTCTCGCCTCCTCCCCCTCACCTCTCCCAGCCGCACCC
GGCCCTGGCCTGCCACCCAGAACTCGCTGGGCAAGTCGTGCCCCGCGTGAACACACAGAAGGGGCTTGGGGACCGAGCGCGGCCCATCAGTCCCCTC
AGACCCTGAGGACCCAGAATTCCCTAAGGGGGTCCGAATCCGAGTCCTGCCCCCAGCCCTTAAGGCACGGGCTCTCCAGGGACCCAGGAAGGGCGCG
GGGCATTAGGTACGCAACCCGTTTCCCCGCACCTGGAAAAAACTCCCTTTCCCTCCCCTCCCCCTGCTTGTTGAGTGTGCGGATAACCAGAACTCTA
AGGCGCCCCGTAATAACGACCCCGCTGTCCCTCCACCCACCCCCAAGTGCCAAAGCGAGGGATGGAAGCGCTTTCAAGCGTTCCAAGGGCATTGAGG
AGCGAGCTGGAGAGGCGCGGGGATGCGGGGTCCTCCCCGCAGTCTTCCGGAAAGGGCGGGGGGAGGGGCGCGGCAAGTCCGGAGTGGGCATGCCGTG
GGGCATTAGGTACGCAACCCGTTTCCCCGCACCTGGAAAAAACTCCCTTTCCCTCCCTCCCTGCTTGTTGAGTGTGCGGATAAACCAGAACTCTCTA
AGGCGCCCCGTAATAACGACCCCGCTGTCCCTCCACCCACCCCAAGTGCCAAAAGCGAGGGATGGAAGCGCTTTCAAGCGTTCCAAGGGCATTGAGG
AGCGAGCTGGAGAGGCGCGGGGATGCGGGGTCCTCCCCCAGTCTTCCGAAAGGGCGGGGGAGGGGCGCGGCAAGTTCCGGAGTGGGGGCATGCCGTC
GGAGCCCACGAGGGCCTCAGCGCGGATCCTCCGCCGGAAAACCGGCTCCCGCGAGCCGCCGCCGCAGGTTTCCTAGGCCCGCGAGTCCCCGCAGCGA
AGCCCTGCGTCTCCGTCCGACGCGGGGGTCTGCTCAGCCTCGGGTGGGGCCGCGGCCAGGCCTGACTGCGGGGAGAGGGCCGAACGTGACCTCCGAG
GTCACCCCCAGCCAGCTTTTCTCTCCTGTGGTCGGAAGTGGTTTTCTTCTCGATCGGGGCGCCTACTCCCCACCCACTTGGTCGAGAGGGCTGGGGC
CGGAAGGCCAGGGAATCTCTGGTGGATTTGGGGGTTCATATTGCTCAGGGTACCAGCCGATGCGTTTGAGGGGGGGCGGGAGTCGAGGTTTAGTTCG
CCTTTAACCCTCAAGAGTTGCGCCTTCAGCCTCGGGATCCCAGATGCGTCGTTGGAGCCAGGGCCGCCCCCCTACCTGTTGGGTTTGCGTTTTAACT
CCAGCGCACACCTTGCCGGCAGCCCTCGGAGCTAGGGGAGGGGTCTCGTTTCCCCGCAGCCCGCCGGACAGACGACTGGGGCACGGGAGGGGCGGTG
GCAGGGTGGTCTGTGTGTGGCTGAAACTAATTGATCTGGAGCGGAAACGCACGTCTGCGGTTGGGGCGATGGGGGGGGCGGTGCGGCTGTCCATGTG
CCGAGCGTGTGGCTGTCTCGGGTGGGCACTGGGGCCGGATTTCGCCCCGGCCCACCTCGCAGTTTTGGGGCGCCTGGGATCGGCGCTACGTAAGCGA
ACCAGAGCTGCCATAGCACGTGGGCCGCCACGCGCACCCCAAAAGCAAGCAGTGTGGGGGGAAGGGGAGCTCGAGCGCCTTCGGAGCCCAGGGGCCG
GCTTTCGGAAGCGTTTTCCCGGGCGACTTAAGGGCTTAACAATGGAAAACTCGCGGAGCCTGAGCCAAGTCCTTTCAAGTCGCCGCCAGGTATGCGG
CTGCAGGTGACCCCACCTGGGTGCGCCCGCCCGCCAGCCGCCCTGGTGGAAAAGCGGGTGCGGGAGGTCGCTGGCGAAAGGTCGGGACTGGTCCCTG
CACCACCCGCCCCCAACCCAAGCCCCGAGCCCCGCGGCGCGCAGCCGCGCTGAGTCCCGGGGTCTGCGTCGCGGCGCGCCGGTTCCTGAATGAACGC
GCTCCCTTCCCCCGCCTGAATGAAGGTTCCCACAGCCAGGGACGGTGGCGAACACGCGCCTGCAGCGGAATTCGCTTTCTCCTGACCGACCATCCGC
CCAGGCCGCGGTCACCGGGGCGGGGGCCTGGGGGCGAGGAAAGCGTGAAGGTGATTTCAGTTAATTTTGGATTTTCTTTCAAACAACGTGGTTACCC
TCCCGACTGGGCCACTTGCGCTTTGTCTCCAAATGGTCACCAAGAAATAAGAACAGAGCACTTTAAATGAGCCCAGAATCCGCAGTTCCTGCTTCGT
GGTGGGTTTTAAGAAGACAGTGTAAAGTAAAACTGCAACCGAAAAGTTTTTTAAAGTTGCTTTTCTCTTTGGAAAAAATAAAATCAAAATGCTTTCT
CTGCGCTTCTTGAAGCAATGACCCTCAAAAGCCCAGAGGTATTGGCCCCCTCGGGGGACCCGGGGGCCGCCAAGCAGGGTTCCCCCAGGTGGGGGCT
GGGCAGCTGGCGCTCCCCGCCGGGCCCCAAATTCCAGCGCCGGGCCCGCAAATTCCAGCGCCTCCCCCGCGGGTTCCTGGACGGCTCTTTACGCTCG
CTAACCGGGCTTGCAATTTTGCGCTCGTCCCTGAGCCGGGAAATCAACGAAGTTCCTAGTCGAGATCTGCCCGGTCCGCCTAGTAACAGCGCCGCGC
CCCCATTGGCTCATGCTAATTCCAGTTTCCTCTGTCTTTGCGCCCGGGATGGGGGGTGAAGCTCCCTCCTGGACCCAGAGCCGGTTGTGCCGGAGTG
GGCGAGCCTCTTTATGCCCTGCTGCCCCTAGCCGACTTCGGCCCGCTTCGCGCCTCGGGCTGGGCCAGGGCGCACGCGGGGCTCGGGGCCCCTCGCC
CCACGGGATGGGAGAGGCCGGGTGATAGCTCCGGGCCCCATAAATCATCCAGGCGGCCGCCGGGTCGGGATTTTATGAATGAAAAAGCAGCTGTGCC
GCCCTTGTGCGCGGGCTGATGCTCTGAGGCTTGGCTATGCGGGGGCCAACGCGATTGTGGGTGCTCGGGGAGTGGGGGGGGGCACGACCGTAGGTGC
TCCCTGCTGGGGCAACCCATCGCTCCCCATGCGGAATCCGGGGGTAATTACCCCCCCAGGACCCGGAATATTACTAATCCTAATTCCCGGCGGGGGA
GGGCGCGCGGGAGGAATTCACCCTGAAAGGTGGGGGTGGGGGGGGTCGCATCTTGCTGTGAGCACCCTGGCGAAGGGGAGAGGGCTTTTTCTATCAG
TTTTCTTTGAGCTTTTACTGTTAAGAGGGTACGGTGGTTTGATGACACTGAACTATATTCAAAAGGAAGTAAATGAACAGTTTTCTTAATTTGGGGC
AGGTACTGTAAAAATAAAAACAAAAGTTAAGACAGTAAAATGTCCTTTTATTTTTTAATGCACCAAAGAGACAGAACCTGTAATTTTAAAAACTGTG
GATTTTAATTTACATCTGCTTAAGTTTGCGATAATATTGGGGACCCTCTCATGTAACCACGAACACCTATCGATTTTGCTAAAAATCAGATCAGTAC
ACTCGTTTTGTTTAATTGATAATTGTTCTGAATTATGCCGGCTCCTGCCAGCCCCCTCACGCTCACGATTCAGTCCCAGGGCAAATTCTAAAGGTGA
AGGGACGTCTACACCCCCAACAAAACCAATTAGGAACCTTCGGTGGTCTTGTCCCAGGCAGAGGGGACTAATATTTCCAGCAATTTTAATTTCTTTT
TAATTAAAAAATTTGAGTCAGAATGGAGATCACTGTTTCTCAGCTTTCCATTCAGAGGTGTGTTTCTCCCGGTTAAATTGCCGGCACGGGAAGGGAG
GGGGTGCAGTTGGGGACCCCCGCAAGGACCGACTGGTCAAGGTAGGAAGGCAGCCCGAAGAGTCTCCAGGCTAGAAGGACAAGATGAAGGAAATGCT
GGCCACCATCTTGGGCTGCTGCTGGAATTTTCGGGCATTTATTTTATTTTATTTTTTGAGCGAGCGCATGCTAAGCTGAAATCCCTTTTACTTTTAG
GGTTACCCCCTTGGGCATTTGCACGACGCCCCTGTGCGCCGGAATGAAACTTGCACAGGGGTTGTGTGCCCGGTCCTCCCCGTTCCITGCATGCTAA
ATTAGTTCTTGCAATTTACACGTGTTAATGAAAATGAAAGAAGATGCAGTCGCTGAGATTCTTTGGCCGTCTGTCCGCCCGTGGGTGCCCTCGTGGC
GTTCTTGGAAATGCGCCCATTCTGCCGGCTTGGATATGGGGTGTCGCCCCGCCCCAGTCACCCCTTCTCGTGGTCTCCCCAGGCTGCGTGTGGCCTG
CCGGCCTTCCTAGTTGTCCCCTACTGCAGAGCCACCTCCACCTCACCCCCTAAATCCCGGGGGACCCACTCGAGGCGGACGGGGCCCCCTGCACCCC
TCTTCCCTGGCGGGGAGAAAGGCTGCAGCGGGGCGATTTGCATTTCTATGAAAACCGGACTACAGGGGCAACTCCGCCGCAGGGCAGGCGCGGCGCC
TCAGGGATGGCTTTTGGGCTCTGCCCCTCGCTGCTCCCGGCGTTTGGCGCCCGCGCCCCCTCCCCCTGCGCCCGCCCCCGCCCCCCTCCCGCTCCCA
TTCTCTGCCGGGCTTTGATCTTTGCTTAACAACAGTAACGTCACACGGACTACAGGGGAGTTTTGTTGAAGTTGCAAAGTCCTGGAGCCTCCAGAGG
GCTGTCGGCGCACTAGCAGCGAGCAGCAGAGTCCGCACGCTCCGGCGAGGGGCAGAAGATCGCGAGGGAGCGCGGGGCAGCAGAAGCGAGAGCCGAG
CGCGGACCCAGCCAGGACCCACAGCCCTCCCCAGCTGCCCAGGAAGAGCCCCAGCCATGGAACACCAGCTCCTGTGCTGCGAAGGGGAAACCATCCG
CCGCGCGTACCCCGATGCCAACCTCCTCAACGACCGGGTGCTGCGGGCCATTGCTGAAGGGGAGGAGACCTGCGCGCCCTCGGTGTCCTACTTCAAA
CAACTTGTTGCCCAGACCCACGTTTCTTTGCTACTCACCCCCCTCCCTTCTCTCCCGCTAGAACTTTGAAGTTTGCCGGGGTGTTTCTAGGGATCCG
TATTTTCAAAATAAAAAATTGCGGGTATTTTCTGAAGGAGGAAGGGTGGGGGTGGGGGTGCTAGAAGTAGCGTTTCGTGGGAGGGGAGAAGGGGGTC
CGGGAGGGGGTGCCTTCGGGGAGAAGCCAGTGCCAGGGGCACCCCAATGGGCCGAGGGTGCGGGCTGGCAGGCTGGGTGCGCTTTGTGTCCCCGCCT
GCGCCCCAGCCCCGCTGCGCCGCAGCGGCCGGGAGCCGCCAACTCCGGGGGGAGGGGGGGCATAGATTTGATTTTTTATTTTTATCCATGGATCGTA
TGCAAGGGGCCGCTCGTGCCAGTATTATGCGCCATCTTTGCTCTTTTTATTGCAAAAGTGTTTATTAATAATTGGGGGCAGGGGTGGCCGGGCGGGG
AGCGGCCGCCGGGCGCTGGGGCCGCAGCTTGGCCGCGCGGCTGCCGGGAGCCCGCGGGAGGGGCGCAGGACGCGGCATGGGTAGTTTTTGTGGGGGG
ACCCCGCTAGGGAGGGGGGGCCTTTGTTCTAGCAGCGAGTCCCGGGCGCCCCGAACGGGCAGCCTGGGCCGGACAGCACGGCGAGCTGCAGGTGGTC
GCGTGGCCCCCAAGACGCCAGGGCTTGATCCCCGTCTGCAGGGATATCGGCTTGGAGACCTTCTCCGAGCGAGCCGTGGCCTGGGAGCATTTTTTTT
CAGACCTTCGGTGGGCGCCTGAGGGGCCCGCAAGTATTTTAAAATAATTTTTGAAAGTGCGGCGTGGTGCCCTTGCGAGAGTTTTTTCGCCGCCTCG
GAGGGTCATTTTCAGGGTTTTTTTATGCACTTAGTTATTTTTTTAATATTTTTATATTTTTTTTGAAGATGACGTCTGGGGGTTTATGCGGCGCGTC
GGCCTGGGCGCCACCTTTGTGTCTCGCAGGCGCGGCGCCCAACCCCGCGGCCCGTTCCGCGGCCCCCGCACCCCAGTTGGTGTCGACCCCCAGTCAG
AGGAACCACGGAGCTCCAGGGCGGGCCAGGGTCCCGGGGGCCGGCAGCCCGCGCCGCCGCGCACCGCCGCCCAGCTGTGCCCGCTCCTCCCCCACCG
TGCCAGCCTCGCGGGGACTTTCCCTTTCAGTTTCGGGGAGGGTGGGTACTGGGGACGCGCGGGGGAGGGGGCGCATCACGGGAAGCTCCTGCCCCCC
CCAGCCCCGACCCCCTCGGCGCCCTCCAGACCTGGCGGCCCTGCCAAGCGCGATGGGGGTGCGGGGGCGTGCGGGGGGCGGGCGCGACCTGGCGGCG
GCGGTCACGGGCCCCGTGCCCCGTAGGTCTGCGAGGAACAGAAGTGCGAGGAGGAGGTCTTCCCGCTGGCCATGAACTACCTGGGACCGCTTCCTGT
CGCTGGAGCCCGTGAAAAGAGCCGCCTGCAGCTGCTGGGGCCACTTGCATGTTCGTGGCCTCTAAGATGAAGGGAGACCATCCCCCCTGACGGCCGA
GAAGCTGTGCATCTACACCGACAACTCCATCCGGCCCGAGGAGCTGCTGGTAACCACTGGACCCCGCCGCCCCCGCCCCCCCGCGAGCCGCACGCAC
GACCACGGGGCCGGGGAAGTGCAGGCGGTGGCGGCCGGCCCGCCTCTAACATATCTGCTCCTCCGAGGGATGCGGCCCTCCGCCCGTCGGGGTCCCT
GTCCGGGGAGCGGGCGGGATCCTAGCCGCCCTCGTCCCGCCGCCCTGTGTGCGCTTGCCTGCGACTCCCACCGCGTTCGCGCCCCGCGGTGTGGCCG
AAAAGTGGGCGGCGCGCGCCCTCCAGCGGCTGCACGAGGAGCGCCGCGCTCGGCGTTGCCTCCAGTTCCAGGTGTGGGAGGTTTTTCTTTTGTTTCC
ACTTGCAGAGTCTTTTCACGCGGCGGGCGCCTTTTCTGTTTTGATCTGGATTGCGTGTTGCCCCAGCTCCCCTTGAGTCCCCAGCATTCGCCAGCCC
TCCCCCTCCAACATCCAGGACCGCACGAGACGCAGGGGCCAGTGCTCTGAGCCGGAGGTGCGGCGTGGCCCGGCCCCGTGCTGCCGGCTTCCCCGCG
CCCCCGGGCTGGCCCGCACCTCCCCTGATGGCCGCTCACCCTGTGTTCGCAGCAAATGGAGCTGCTCCTGGTGAACAAGCTCAAGTGGAACCTGGCC
GCAATGACCCCGCACGATTTCATTGAACACTTCCTCTCCAAATGCCAGAGGCGGAGGAGAACAAACAGATCATCCGCAAACACGCGCAGACCCTTCG
TTGCCCTCTGTGCCAAGGTAGGGCAGGCCCGGCAGCCCCCGGCCTCCCCCCCTTGATGCCGGCTCCTTAGGTGACCCTTCCGGCTTCTTGCTCTCCA
CCTGGGTGCTGTCTGGGAAGATGTCCCCAGACCCCTCCTGCGCTGGAGAGCGCTCTTCCAGCTCTGGTGAGCAGAGGCCCTGGATTGTTTTGTCGCG
CTGGATGGAGGGAGATTTGCTCCCTCACGGCCACCATGCAGTACCTTGGGCATTGGTGTGGACGGCTCAGCCTGCCTGTGTCCCGTTACTCTGGCCT
CGTCCTTCAGGCCAGGCAGCCTGTGGCCACTCCATGCTGAAAGGGGTTTACCTTGGCCACAGGGCCGCCCTCCTTTCTCCACCACCTCCAGCCCTTC
TTGTTCCTTAAGGAGCCTGAGCTGCAGAGGCCCCCCTCCTGGCCTCTCCCAGGCTGGGCCACCTGCCAGAGGCGCCTCCAGGGGCGGGGAGAGCTGT
CGGCCTGCCTGCACCACGTGCTCTGGGCAGCGAGTGCAGGGGGTGTCCAGCAGAGGAGCTCGGCTGCCTGAGGCCCTGCCAGGGGGTGCCGGAGCCA
GCCGGGCTCAGCTGAGCCCTGAGGGGGCGCTTCAGAGCACTCTCAGCTTGGGCCGCCACCGTGGGCAGCAGAAGCACCCAGTCCTCACTTCCCCTGG
CATGGCCCCAGAGGCCCCTCCCTGACATGGCCTTGGCCCCAGAACCCAGTGGGGACAGACGCGCACATACACAGGGTGCCGCCTCCTGCTGTCCCCA
GCCCTGCCTCTGACCCCCCTGTGACCGCCTCCTTCCCTGGCCCAGGAGGCCTGGTTACCTTCATGGGGGAGCATGGCCCCATCCCACCCAGCTCTGC
TGTGGCCCACCTTTGGTCAAGCCTCAGTTGTCACATCTGTTTGGGGGCTCACTCTGGGTGACCTAGGCCACAAGGCCCACGGGGCATCAAAGAGGCA
GTAGCATCTTCTCCCCTCCCCAGAGGGCAGAGCCCCCCAAGCCTACTTCAGAGCTCCCTTCTGACACCGGTAGCCCGCAGCCGGTATTCCAGAATGG
GTTCTGGTTTAGGCGTGAGGCCTCCCCCACCTCCTCCACCTGCTTGGGGCATGAACCCCTCCCCCACGTTTCCAAGCGAGTCCCCAAGGTGGGCAGA
TGAAGATGCCAAGGATGTCGACCAGTCTGGATGGGTCTGGGGTGGGGGGGCATGCGGCAGACAGGGAGGCATTCTCTGGCTGGTGCTCCTCAGAGGA
GAGAGCCTCCGGAGACTCCAGACAGCCTTTTATGGGCTGAAAGTGGCTTCAGAGAAATGCAAAGTTTCCTGGAGAGAACGTGGGGGCGTGGTTTCTT
TCACAGCCTCCCTACAGGGTGGCTCCAGCAGTGGAGCTCCCCTCCCAGGACCCCTGGGTGCTAGTGGGAGGCTGTGGGCAGGTGCAGATTCTCGTCC
TTCCCACTACTGCACACCCTTTGTCTGCGAAGGCGCCCCCAGCGGTGGGTGAAGGAGGAGGGACACTTGGGGACCCAGCTGTGCACGTGCTCTCAGT
GACTGTGGAGTCCACTCCAGGGTGGGTCCCGAGGGAGGGGCAGGAGACCAGGGGACCCACCCCTGCAAAGTGCTCCGGGTCCTGACCCGTGGCCACC
CCATGGAACGTAACTGAGCAGCCAGTGCCTTGTTCCTGCTGGACATCTGTGGAGACAAGAGTGACTTACGGCGGCTTAAAGTCAGAAACAGGTTGAA
GGAGGTGGAGGCGTGGGAAGAGTCTAGGAAGGTGTTTTTGCCCTCCACGTGGCAAAGGTTACATTTAAAGGTGATGCTGGGTGTTCTTCCCTGCACT
AGGCATTCCTGGCCCCAGGTCCCCAGCAGGTGTGCACATGCTGCATACACTCACGCATGGGGGTTTCAGGGCAGGTGCGCCCTTGGCTCCGTGGGAG
GCCAGGTGAGGAACGTCCAGTGCCAAGGAGCTTCCGGGACAGCTGTCACTTCCCTTTACAACCAGGCACCGGATAGGGTCAAATCCTGGAGCTTTGG
TTGTCTAATTCTGGGTGGCTCCTAATCTAAGCACAGACAGCACCACACACTGGGTGGGGGCACGAGCTTCTGAAACAACGTGGCCCCAGTGACTCCA
CGCTGTGTGTGCCCCTGGAGACGGGGGGTGCACAAGGTGCGGAGCCAGCTAGAACCTGTCGCTCCCTGCAGAAGCGGTTTTCTGTGTGCGGTTCTGA
TTTTGCCTCAATGAGAAGGTTTTCATTCATGGCTCCCGGCTCTCAGACTGGGTGGAACTGCTCCCATTTAAAGGGGAAAGAGGTGGCTCGGCTCGTT
AAGGATTTTCTTTTTCTAAGTTGTTTACGGCGCCCAGCAGCCGGCTTTGTCTCCCCTTCAGGGTGGCTGCCTTTCTTCCCGCCCTCGCCGGCGGCCC
TCTCTTTAACAAGGCCGAAGTTGTTTATTCTCTCGGGATGAAGTCTCGGATGGGCCGCCACACCCCTGGCGGCCCGTGGGGGCCCCTCTCCCTTTGT
GCCTGGGTCGGCTCCCATTCAGCTCCCCCGACCCCCCTTGTTCCCGGGCGCTCAGTGGCGCGAGATGAGGCGATGGGGCCGACAAAGATGCCACACT
CATCCCTGCCGACCTCCGGCTCCCAGCCCAGGGCCCCTGGTTCCTGTGCAGAATTCCTCGTGGGTGTGACAAAAGGCTGCCCCCAGGCTCCGCTGGG
GTGGGGGCCAGGCCAAGAGGCACATCCCACACTGGCCCACCTGTCCACGGTAGGCGCATGACTGCCCTGAGGAGGGGAGGCCGGCATTCCCCGCCAC
AAACCAGGACGTAATTGGTGGCAGGGCTCTCTGTGGAAAGAGCCAGTCTGCTGTTTGTCTAGGAGGTCAGTCACAGAGGCCCCGAGACGCCCACTAC
TGCAGCCTGGCAGGCGGATGAGCCCAGTATCTGGCAGTGACCAGAGGGAGTTTTGTGCAGACCACAAAGGCTGATGGGCTGCCCTAGATTGGTGTCC
CTCTTGGAAGTGGGCCCAGATGTGCGGGACAGTCCCCAGGAAGCCCCAGGTGAGGGCACTGGTGCCCTCTTGGGAAAGCTGCTCCCTCCTGGGGCCC
GGCTCCCGGCCCAGTCCTCCAGGGGTGTCCCATGGTGACTGGTGCTAGGAACCCCACACCTCTTCCCTTACTTGGGAAGTCACTGGAATTGTTGGGC
TACATCAGACGGCCCAGAAAAGTGTTTTTGTCATCGGCCAGAAATAGGAGAGTTGTGAGTAGAGGGCCCGGGTGGAGTTGGGGTGTACTTGGTCTGT
GCTCTGAAGGTCACTGTGACAGTCATGGTCCCATGGTAAGGGGCATGGGTTGCTGGAAGACCTCTTCCTTCCCGAGTGAGCCAAGCCGGGCTCTCCT
GGCGCCAGGGCCTGAGCCGCAGCCACACCACAGCCGCCCTGAAGGCTGCCGGCCAGGGCTTACCCCTCAAGGGACACGGAATGGCTTCATCAGTACC
CTGCAGCCCCGTGGCCTGGCCCGGGTGGAGGCCTAGGCTTCAGCCATGCGATGTCCCTTCAGAATATGACTTGTCTGCAATCCCTGCTGCTGGGGGG
TTGGCAGGTACTTGGGGTGAGGGTTAGGGTCATAGAAGCGACATCTCTACGTCCTCATATTTGCGTCATCTAATTTGTTTTTGTGAATACGTGATAA
CATTCACAAGGCTCAAGATGCTAAAAGGATGAGAAGGCAGTGATGTCCCCATCACCTGTCCTGTGTCTTCCCGTGGCTTTCTCTTTCCTTGGTTATG
TTTGAGTCAACAGTGCGGCTGACGTTCCAGGAGGGTCCGTGGGCCAGGCTCTTGCTCTCCGAGTGCCCAGGGATGGCTGGAGGCTGAGGAGGGCCTG
GATGTGGAGCCTCAGATACCAAGTGCTTCCCTTCAGGCCGGGCCGCTTGCTCAGAGCCAGCACACAGGGATGCCCGGATCACGGGGGCCCTGAGAGG
GTCCCCTGCTCACAGCCTCCTTCCCTCTCTCCTTCTGCCTCAGATGTGAAGTTCATTTCCAATCCGCCCTCCATGGTGGCAGCGGGGAGCGTGGTGG
CCGCAGTGCAAGGCCTGAACCTGAGGAGCCCCAACAACTTCCTGTCCTACTACCGCCTCACACGCTTCCTCTCCAGAGTGATCAAGTGTGACCCAGT
TAGTGAGGGTGATGTCCCAGGCAGCCTTGCCGGGGCTTACAGGGGGAGACACCTAGTGCCACGGAAATGCCGAGGCTGGTGCCAACGCCCCCAAGGG
TGACAAGGTTGGGGCTGGGGCTGGGCCCCTCGGACCCCAGGCCACAGACTGACAGGGCACCGGCTTCTTCCACTGCTCCTAGAACTTACTGACTGGC
TGGGAGGTCCTCACAGCCTTCTCACGTCCCCTGGGGCTTCCAGGAGCCGTAGAGTTTCTGGGCGAAGCGTCCCGGACGGAGGCCCCAGGCGGCCCCA
GCCAATGGTCTGTGTGGTGATGGTGTGTGGGGTTAGGCCCAGGCGAGCTTTGTTTGGGCCACAATGTGCGTGGCCAATAAATAGATGCTTGAAAAGG
GCTCCTGTGAGGTCCGAGACACCGGACAACGGGCGGATAGAGACAGCCTTGTTGTTTACGGCCTCTTTGAGAGGCTGCTGCTGTTAAACCCTGGGAT
GACTGTGTCTTTCTTCTTAAAAATGCCATTGTTTTATTCCCGAGTCTTTTCTTAAAGAAAGAATTAAAATGACAATCAAAAGGGTTTGTGGCATTTA
CCAAATTAGACCAGAGAGGTGGCCGGGTCAGCCGCCGGCCCCGCGGTGTGTGAGGGAGTGACCGCCTGACCCCAGCTTGGGGCTGGGTGGGCCTGCA
AGACCCGTTTTGGCTCTGGCCTGGGCCGCCTCTTGGTGGTCTGCCCTCGAGCCTCCCGGGGACTCCGCACGGGTCTCAGCAGATGCTATCTAGGGTC
CACCTGCCTGTCCCCTGCCTAGTGGTGCCTCTGTCCCGGGGACACTGGGAGTAGCGGCTGCCCAGCCCATGTGTGTCTCGGAAGAGGAAGAAGCTTT
TTTGCCGTGGGACACCGAAGTTGGCAGGGGCCTCCCTTCTGTGTTCTCGGCCATGGCCTCCCTTGCACCCTGCCCCGTGTTATCCTTTGGGGGTGGT
GAGGTGTCCTCACCCGCTGTAGGGTGGAGGCCAGCAGCCCGCAGCTCTCTCAGGAAAATGGCTCAGAAACACCATCGAGGCCTCCAGAAGCCCAGCA
AAGAGAAAGCCCCTCCATCAAAATGAAACTCGCGTCTGCACTTTTCATTTCGAACTCCACGCCCTGAGTGAAAACCGCTTCCCCGCCAGGGGTGACT
GCCCTGGGATGTTGCTGTCTTCGGGCAGTTGTGGGAAGTTGGGCGCTGGCCCTTATTTGAGTAGAGACCATCTTAACTAGATTGGAGGCACACGTCT
CACAGCTGACAGACACACGGGGTGAAGTTACCCGAGGCGGAGTCCACTCTGCCTGATCAGCTAGTGACCAACGTAGCTGAGCCCAGACTCAGAAAAA
CCGTCCACAGCAGAGGCCCCTGCATTTTCTAGGGCGTGTTCTAGAATTTTCTTTGGTGGGTGGAATGTCCATCTGTGCAAATCGGGTGCGCAGTGCC
TGTGGGGCCAGAGGAATGAAGGAAGGTCCTGGAGTCTAGCTGCATGTGTGACCCTGGAGTGGGTCATGGGCGAGGGACGGGCCGCAGGTGAAGAATC
CCTGGATGGAGCTGCCAGGCCCCTGGGGCTGAGAATTGAAGCTGGCTGGTGTTTTAGGTTGAACGTCAGGAGTCTTGTATCTCACCCCAGGCCTCTG
GCCTAGTTTCCCCATCTGTACAGTGGGACTGTTTGTGCAGCCAGCCCGGCCAGCTTCATTTGCCATGATGAGAATTTATCTGAGGGGGCGGGAGAGG
AAAGCCCTCCCTATAAAGGTACAGGCGCTAAAATGTCATGACCTCAGTGGTCCACCTTTGTCGTTCTGTCCTGGGTTATCGCCATGTCGTGCATATG
CCTTTGTCCAGCCCCTTCTGGTTGGGAGTTAAGTGGCACCTGTGCGGCACGTGGTGGGGCTGTGGCCCAGCCCTGCTCCTTGTGGAAGGTCTGTTTC
CTGGGCTGCCTAGAGACTTGGCTTGAAGCCCTAGCGTGGCTTCCTGGCAGTTGGGACACACACAGCCCCAACACATGGAGCCGGTTCTCCATCCAGA
AGCCCCCGGGCAGTAAGCAGCCACTTTCAGGCTGCGTGGGACTTGCCCGTGGTGGAGCCTAGGAGAGGCCCTGGCTGGGCGTGGCGTTCCAGATTTC
ACGGCTGCTCTTTCCCACTGACAGTGTGGTGTGGACGCTGCCAAGGGAGTCTGGAGCCCCAGAGGGTGAGGTGCAGGACTTTCCAGGAGCGTCCGTC
GCACTCCACCCGAGGGCGAGCACCTCAGTGGCCGCAGTGGGTGGATGCATGCTGTGCCAGGCTGATGGCTGGCCCCCGGGGCACAGGCCTGAGCGGA
GAGGATGGAGGGGAGGGATCAATGGTCCAGGTCCCCCCTGGCCACCCCAGCATTCATCCTCAGTCATGCACGGCCCTTCTTCGACAGCCATTGATCA
TGGAAGGCCAGGTTCACCTCAAGGGCTGCCACATGGAGAGGTTAAGTCTGAAAAGGCTGAAAAGGCAGGGTTAAAGGGGCCTCCTGTCCAGATCAGA
TGGCAGCATCTGGTGTCTTGTGGCTCCAGAGACCAGGTGGGAGGTGGAGGCGTTTTGGGTCCGAGTGTGTCCTACAGGTGATGGTGCTCCCACATCT
GGGCAAGCATCTGGTGTCTTGTGGCTCCAGAGACCAGGTGGGAGGTGGAGGCGTTTGGTCCTGAGTGTCCTGACAGGTGATGGCAGCTCCCACATCT
CCTCGCTGCAGGCCCCTTCTAAGGACCCCCTCTTCCCACCTCTCCCCACCCTCTCTCTCTCAGGACTGCCCTCCGGGCCTGCCAGGAGCAGACCGTG
CCCTGCTGGAGTCAAGCCTGCGCCAGGCCCAGCAGATCATGGACCCCGGCCGCCGAGGAGGAGGTCAGGAGGAGGGAAGGAGGAGGTGTCCTTCTTG
CACACCCACCGACGTGCGGGACGTGGACATCTGAGGGCGCCAGGAGGCGGGCGCCACCGCCACCCGCAGCGAGGGCGGAGCCGGCCCCAGGTGCTTG
CACTGACAGTCCCTCCTCTCCGGAGCATTTTGATACCAGATGGGAATGCTTCATTCTCCTTGTTGTTGGTTGTTTTTTTCCTTTGCTTTTCCCCCTT
CCATCTCTGACTTAAGCAAAAGAAAAAGATTACCCAAAACTGTCTTTAAAAGAGAGAGAGAGAAAAAAAAAAATAGTATTTGCATAACCCTGAGCGG
TGTGGGAGGAGGGTTGTGCTACAGATGATAGAGGATTTTATACCCCAATAATCAACTCGTTTTTATATTAATGTACTTGTTTCTCTGTTGTAAGAAT
AGGCATTAACACAAAGGAGGCGTCTCGGGAGAGGATTAGGTTCCATCCTTTACGTGTTTAAAAAAAAGCATAAAAACATTTTAAAAACATAGAAAAA
TTCAGCAAACCATTTTTAAAGTAGAAGAGGGTTTTAGGTAGAAAAACATATTCTTGTGCTTTTCCTGATAAAGCACAGCTGTAGTGGGGTTCTAGGC
ATCTCTGTACTTTGCTTGCTCATATGCATGTAGTCACTTTATAAGTCATTGTATGTTATTATATTCCGTAGGTAGATGTGTAACCTCTTCACCTTAT
TCATGGCTGAAGTCACCTCTTGGTTACAGTAGCGTAGCGTGGCCGTGTGCATGTCCTTTGCGCCTGTGACCACCACCCCAACAAACCATCCAGTGAC
AAACCATCCAGTGGAGGTTTGTCGGGCACCAGCCAGCGTAGCAGGGTCGGGAAAGGCCACCTGTCCCACTCCTACGATACGCTACTATAAAGAGAAG
ACGAAATAGTGACATAATATTTTCTATTTTTATACTCTTCCTATTTTTGTAGTGACCTGTTTATGAGATGCTGGTTTTCTACCCAACGGCCCTGCAG
CCAGCTCACGTCCAGGTTCAACCCACAGCTACTTGGTTTGTGTTCTTCTTCATATTCTAAAACCATTCCATTTCCAAGCACTTTCAGTCCAATAGGT
GTAGGAAATAGCGCTGTTTTTGTTGTGTGTGCAGGGAGGGCAGTTTTCTAATGGAATGGTTTGGGAATATCCATGTACTTGTTTGCAAGCAGGACTT
TGAGGCAAGTGTGGGCCACTGTGGTGGCAGTGGAGGTGGGGTGTTTGGGAGGCTGCGTGCCAGTCAAGAAGAAAAAGGTTTGCATTCTCACATTGCC
AGGATGATAAGTTCCTTTCCTTTTCTTTAAAGAAGTTGAAGTTTAGGAATCCTTTGGTGCCAACTGGTGTTTGAAAGTAGGGACCTCAGAGGTTTAC
CTAGAGAACAGGTGGTTTTTAAGGGTTATCTTAGATGTTTCACACCGGAAGGTTTTTAAACACTAAAATATATAATTTATAGTTAAGGCTAAAAAGT
ATATTTATTGCAGAGGATGTTCATAAGGCCAGTATGATTTATAAATGCAATCTCCCCTTGATTTAAACACACAGATACACACACACACACACACACA
CACACAAACCTTCTGCCTTTGATGTTACAGATTTAATACAGTTTATTTTTAAAGATAGATCCTTTTATAGGTGAGAAAAAAACAATCTGGAAGAAAA
AAACCACACAAAGACATTGATTCAGCCTGTTTGGCGTTTCCCAGAGTCATCTGATTGGACAGGCATGGGTGCAAGGAAAATTAGGGTACTCAACCTA
AGTTCGGTTCCGATGAATTCTTATCCCCTGCCCCTTCCTTGAAAAAACTTAGTGACAAAATAGACAATTTGCACATCTTGGCTATGTAATTCTTGTA
ATTTTTATTTAGGAAGTGTTGAAGGGAGGTGGCAAGAGTGTGGAGGCTGACGTGTGAGGGAGGACAGGCGGGAGGAGGTGTGAGGAGGAGGCTCCCC
AGGGGAAGGGGCGGTGCCCACACCGGGGACAGGCCGCAGCTCCATTTTCTTATTGCGCTGCTACCGTTGACTTCCAGGCACGGTTTGGAAATATTCA
CATCGCTTCTGTGTATCTCTTTCACATTGTTTGCTGCTATTGGAGGATCAGTTTTTTGTTTTACAATGTCATATACTGCCATGTACTTGTTTTAGTT
TTCTCTTAGAACATTGTATTACAGATGCCTTTTTTTGTAGTTTTTTTTTTTTTATGTGATCAATTTTGACTTAATGTGATTACTGCTCTATTCCAAA
AAGGTTGCTGTTTCACAATACCTCATGCTTTCACTTAGCCATGGTGGACCAGCGGGCAGGTTCTGCCTGCTTTGGCGGGCAGACACGCGGGCGCGAT
CCCACACAGGCTGGCGGGGGCCGGCCCCGAGGCCGCGTGCGTGAGAACCGCGCCGGTGTCCCCAGAGACCAGGCTGTGTCCCTCTTCTCTTCCCTGC
GCCTGTGATGCTGGGCACTTCATCTGATCGGGGGCGTAGCATCATAGTAGTTTTTACAGCTGTGTTATTCTTTGCGTGTAGCTATGGAAGTTGCATA
ATTATTATTATTATTATTATAACAAGTGTGTCTTACGTGCCACCACGGCGTTGTACCTGTAGGACTCTCATTCGGGATGATTGGAATAGCTTCTGGA
ATTTGTTCAAGTTTTGGGTATGTTTAATCTGTTATGTACTAGTGTTCTGTTTGTTATTGTTTTGTTAATTACACCATTATGCTAATTTAAAGAGACT
CCAAATCTCAATGAAGCCAGCTCACAGTGCTGTGTGCCCCGGTCACCTAGCAAGCTGCCGAACCAAAAGAATTTGCACCCCGCTGCGGGCCCACGTG
GTTGGGGCCCTGCCCTGGCAGGGTCATCCTGTGCTCGGAGGCCATCTCGGGCACAGGCCCACCCCGCCCCACCCCTCCAGAACACGGCTCACGCTTA
GCTCAACCATCCTGGCTGCGTCGTCTGTCTGAACCACGCGGGGGCCTTGAGGGACGCTTTGTCTGTCGTGATGGGGCAAGGGCACAAGTCCTGGATG
TTGTGTGTATCGAGAGGCCAAGGCTGGTGGCAAGTTGCACGGGGCACAGCGGAGTCTGTCCTGTGACGCGCAAGTCTGAGGGTCTGGGCGGCGGGCG
GCTGGGTCTGTGCATTTCTGGTTGCACCGCGGCGCTTCCCAGCACCAACATGTAACCGGCATGTTTCCAGCAGAAGACAAAAAGACAAACATGAAAG
TCTAGAAATAAAACTGGTAAAACCCCAGCGTGGTGCCTGCCTCTTTGCTTCCTGGGCTGGCCGTGAGCCAGGGACGCGTGTCCTGGTGCCCTAGAAC
CAGGGCAGGGTGGCAGGCTTGGCGGATGTGGGAGGCCGCAGCCTGTCCTGTGCGCTGTGGGAAGTTCAGCAGCATCCTGACCTCCATCCCCGGGATG
ACAGTCACGCCACCCGCCGTGACAACCAAGAATGTCTCCTGACACTGCCACATCCCCGGGGGTGGGGACAGAATCCAGCCAGGAGCAGGCACACCCC
TCCCAACTGGGAGGAAGCCCTCAGCACAGGTGTGTGAGGTGGGAGGCGGTGTCCTGTCCCCGGGAGGCTCCAGAGAATTTTTTGCAGGCTGCCTGGC
TGGGTGAGCCCACCTCCAACCACGCGAGACAACAGCTCCGGCCTGGGTGACGTGAGCGGTGCCCATTGATGGGGAACATCTTCCCCCTCTTCCTTGC
CCCACCAGTTTGTCTTCCCGGGTTATTTGCAGATAGGAAAATAAATAAAGCCGGCATTCGTTAACCCTCTTCTGGCGCAAACTGCTGTTTGCTCTGG
ATGAATCATGGTCCTTTGGCGACGCCAGGCTCCGGGAGAGCAAAGCACCGTGTCAGGGCCATGATCCGGGGTGGCCTTTCACTGGGATCGTGGGGAC
CTGGAGGCCGCCTTATAGGACACCCATGACGCCCACCTCTGGATTTCAGGTGCACGTGACTGGACTTAACTTCAAACCCCAGGGTGGAGGCAGGTAG
TGGGAGTGCCCTGGGAAGGTGTCCTCGGACCTTGGTCACTGCTCCTGAACCCATCTGTGAGGCTGGTTTGTCCTCATCCCAAGCTAAGTGGAAGCTC
AGGTCCCAAGCCACCGATGGGTGCTACTTGTCAGCTGCAGGTTGAATCTCCGTGGCCTTTATGAAGCACCTGCTGTCTACCCTTCCTGCCTTGTAGA
GCACTCCTCCCAGGGCTCAACAGTGGGGCCGGGGTGGTCGGTGGGTTGGCTCCACAGGCGCCTGCCCTGGGAGGAAGGTGGGGTGTGGAGGGAAACC
CTGGCCCCTGTAGGTCTCCACCAGCCTCTCCCCTGAGGGTGGGGGGCTCCGGGAGCCTTCCTCGAGGGAGTCCTATATTGAGTGGGTGGGGGAGCCT
GCAAGGTGCCCCTGACAGGTCACATCAGAAAGAGCTCAAGGGACAGTCGGAGCCAGAGGTGACACTGGTGGCCACTCGGGTGGCTCACAAGGCCCAG
CTCCTCCTTGCTCCTGGCAATTACTCTGAAGGCAGGGACCAGGTCTGCACCATTGCGGCTCTCCAGTTCCAGGGGCAATGGCCAGGTCCTGTGTCAG
GGCTGGGGTCCTAGGGAAGCCATGTCCCCACCCCCGGCCTGCAGCTGGGTTTACATTCATCCCCCGAGAGCACATGGGTGTAGCAGGAGGCCTGTGC
AGAGAGCTCCGACCATCGCACAGGGCACCTTTGGTTGTTTCACGGAGCAGGCAAGGGAGCCATCGGATCCTGTTAGGTTTGAGCAAGGATGTGGGGA
AGAAGCTGGAGAGCCACTTTGCCATGCAGGGAGAGGAGCACATGGGTCTAGGGATCTACTTTAGTGTTTGGAAGGTTTTTTAAGATGAAAGAGGGAT
GTGTAGGCTGATAGGTCTGGCAGAGCCAAAAGGCAGCGACATGTCTACTGGGAGAGATGGAGCTGAGCGCGGGGCTCAGGCAGGGTGGCAGGGCAGG
GCCGGGGCCCTGGGTGGGTCAGGTGGGTTCACAGCCAAGTGTGTAGAGAGGGCTTGGGCCCAGAGTGAAGCAGTTGCAAGCTCTCCCACAACCCATT
CTCTCTGTCTCGGCATCTGTGGCATCCCGTGATGGGTGGGTCTGTACACACCCCACCCCTGGCTGTGCCACAATGGGGGTGTCTGTACACCCCTCAC
CCCTGCTGTGCCACGATGGGGGGTCTGTACACCCCCCACATCCCTGGCTGTGCCACGATGGGGGGTTCTGTACATCCCCCCAGTGATGGGTGTTCTA
TACATCGTGGTTATGCCACGAGCTCCAAGGCTGTATCAGTCCGTTTTCACACTGCTAACAAAGACATACCCGAGACTGGTAATTTATAAAGAAAAAA
GAGGTTTATAGACTCACAGTTCTACGTAGCTGGGAGGCCTCACCATCACGGCGGAAGGCGGGGGCCCGTCTTCTTCATGGCAGCAGGCAAGAGAGAA
TTCAATCACCTCCCACCGGGTCCCTCCCACAACACGTGGGAATTATGGGAGCTACAATTCAAGATGAGATTTGGGTGGGGACACAGCCAAACCATAT
CAAAGGCATCGTCTGGCCACTCTAGGCCCTTGTTCCCAACTCTCTCTCCACTGACATCACCTCTGTGCCCTGCTCCCCCTCCTGGGGGATTCAGTTA
GACCTCGTCCCTTGTTGGAGTATCTGCGCTACCAGAGGGTGATGGTAAAACTGACCTTTGAGGAACTTCCCAGAGACAAAGCCTTCCGCAGTCAGCT
CTCCCTGCAGCCCCCTTCGCAGAAGCCTGTCCTCACACGCCCGCCACCCTACCTGCCCTGCCTCCTGGCCCATCTCCTGCAGCCAGCAGAGTCCTCA
TCCATTCTTCATCCTCTGCCCCCAACCCCCAGCCCTTCCTATCTCCCTTCAGCTCCGTCCAAGGTCCCTGGGACTCAGCCCCATCTCTCCCCAGCTT
GACCCCAATAAGTCGATGTCTTTGCCCAGGAGCTCCTGATCACCAGACTGCATCCAGCTTCCTGTTCTGCGCTGGTGGCCTCCCGCCTCGGTCCACA
GGAACCTTCATCTCTGCTCTGACTGTAAGGATGGGCTCTCTGAGGGCTTCCCCACCCCCACAGCAGGCAGGTGGTTCTGGAGTCTGGGATTGTCATG
CCTGGGCACCTTCCTGCTCTGGCTGAGCCCCTCCAGGACGGTTCTCCCAGATTTTTTCTCTGCCTCCGTCTCCCCACCCACCTGGCATATGTGGTCC
TAGGAAGGGGGGCCGAATGGAGGGAGGTCGTGTCGGCTCCTACAGGGCAGCCCCAAGCTGCTGGGCTGAGGTGCAGAGCCATGGGTCCAGCCCTGAG
CGCATGCCGGGAGCTGATTCCATGGCTGGAAGGGTCGGGGGTTCCCCAGGAGCCCAGACAGCACCTAGAGTTGTTCTTTTGGAGTTGGGTCCCTGAG
GGGACAGAGGAAGGAGGCTGGGCAGAGTGGGCACGTGGACTTCGGACGCGCTTGTGGGATAACAGTGGCTAGGGCTGTGCAGAGGGGTGGTGCACTG
CCCATGGGGAGTCCAGAGAAGCCAGGTCCAGTGGCAGTGGCATGCATCCGGCAATCCTCCGGCTACCCTGCATGCAGGCGTGACCTCCTTGAAGCAC
CCAGGAAGCAGTCACAGCCTCGCTCTGCCCAGCATTGGGGCCTGGCCTCTGAATCTGGTGGGGAAGGCAATGTGTGCCTGCTGGCACCCCGCTCTAT
CACCAGTCCCACCATAAGGCTAGTCCCCAAAGTCAGCACTGCAGCCAGGGCCAGCGAGGGCCAGCAAGGGCAGGCCTGTGCAGGGGCTGCAGCGTGG
GGTGTGCGTCCCCGCACTCTGGGCCAGCCTCGCAGCTCACAGGGCTCTGTCATCCCAGGAATGTCTTTGTCAAGAGTTTATGCAAGACGAGGAAAGC
AAGCCCCAGGACCTCTAATCCGTTCCCACAGTCCCCTGCCCGCCAGGGCCCCACAAGCCCAGCCCACACCGCCAGGCCTGAGCAGACAATAGCAGGC
GGACGGGTGGGCGGGCAGCTCTGGGGCCGCTGGCCTTGGGGCCCATCCGGGAGCATTGTCACCGCCACTCACGAGGGCACACACAAGGAGGGCTGGG
GAAGAGAGAATGCAGAGCCCTCTGCTGAAGGGGCTTCGGTGGGCCTTGAGCCTTTCAAAGACGGCGGTGTCCATTAATCCTTTTAATTTGCACCTAG
CGGCAGCTGAGACAAGCCTCTCAACCGGCAGCCACTTTAAGAGCTGAGTGGTCTAACCCGAGGATCAGCTCCTATTCCAGACCCTGGGCCCTCTGAG
CCTGTCGAGGCAGTCCTCCTTGCCTTTGTTTAATCACGGGAGGAATCGCAGCTGGAGGTTACTGAGCACGCACTGTGTGCCTTGTGCTCAACACCGA
ACACAGTTTCTCTCTGAGTTTTGCAACGACCCCATGGGTTGGTGACAGGGAGGGGACACTCTGGTGTCAGCCACAGACACAGCCCTGCCCTCCCAGA
GCCTACACTCTCCCGCACACAATCATCCCATCTGCTAGGTCTGGGATTGGCAGGTTTTCTGGAAAGGGCCACAGAGAGAGGCTTCAGGCTTTGTGGG
CCACAGGTTCTCTTGCCCTGTGCAAAAGCAGCTGCAGCCGCTCTGTGAATGAGTGAGCATGGCTGTGTGCCAATAAAGCTTTATTTACAAAAATAGG
CAGCAGACTGGATTCAGGCTGAGGGTGGTAGTTTGCCGACCCCTGTTGTAGATGAGTAAACCGAGGCCCAGAGAGGTAAAGTAACTGAGCCAGGACT
GCACAGCTAGTGTGTGACCAAGCCAGCCTGGTGGGTCAGCCTCATGGAGAAGAAAAGGCTCAAGCTTCAAAAAGGGAAATGCACGGAGGCTTCATTG
GGCCATTTGCAGCCAGGCCCCACGCACACAGCTCAGGGCCCTGCTTCGCCAGCTCCACCTGTTTCGAGGCTCCCAGTAGGTTGCCCCTGCGGGCGGC
ACTGTGTCTTGGCTGCTGGCCTGGAGAAGGGCAGCCAAACAGGCCGGCAGTGGACATCTGAGGGTGACCAGCTGTTTAAAAAGAAAACTCACCGGCA
TGTATAGGCACACATTAAGCACACACGTTTTGGAGTAAAAGGCCCTGGGGTTGGATCTCGGATCTGCAGCTTTCCAGCTGTGCGGCCTCATTTCTCT
GGGCCTCAATGTTCACACCTATAACATGGGGAAATCCTGCAAGAACACAGCCCATGATGGGGAGTCGTCTCCATCTCCTACTCACGACAACGACCAG
CACACAACGGGGGCTCCATAAATCTCTGTCAATAACTGAATTCTTTCTGGGGTGCTGAGGATTCTATGGCTCAATGTGTGTGAAGCACTGCTGAGCA
CCGAGCTTGGGGTGCAGTGATCACTCAGGGAACGCCATCATCCCCTCGGCGCATCCGCATAATCACCATCAGCATTCCATGCACTGCCCAGGTCTGG
AAGCACAGGCCTCCATGCCACGGCGGCCGCCTGGGTGTTGGCACCAAAGCCGATCTTACTTCCCTCCTTTGGCACATTAAGTAGACAGTTCTGGGTC
TTCTTGACACCACGGATGCCCCCTCCTGGTGTTGACAGCACCCAGACTTTGCTCCAGGGAACCACCATTCCCAGATCTGTGGTTTGAGTGGGACCGA
ACCCATGCCCAGCTCCAGGCCAGGTCTTCATAGGCTGAAACCCAACTGCACATCCACACCCACCAGAAGAGCAGCAAGTTAATGGATGGGTGAGAAA
TCAAATAGAGCCATTGAGTCTTGAGGAGACATTTTCTGGAGCTTCTGAAAAAGATAATGCTTCCCCCTTCCTTAGGATTCCCAGCAGGGAAACACAA
GCCCAGGGGTGGCACTGCCCCCCTGCAGCCCTAAGGGAAGACTGAGCTGCTCCCAGGACCTCACAGCGGAGGCTGAGCGTAAGGCCAGCCCTGAGGG
AAAGCAGGGAATGGGTCTCCAGACAAACTGCTGGATCAAGCCTCTCCTGAAGTCATCCCCCTTTTGTTCCAGAAGCCAATCATTGTCCTTCATTAAG
TCAGTTTGATATGGGCACTGTGTCATTTGGTTTTCAAAGACACTTAACTATTACAGCTTGTGTGTGTTTTCTTTTTTTTTTTTGAGACGGAGTCTCA
CTCTCGCCCAGGCTGGAGTGCAGTGGCCTGCTCTCGGCTCACTGCAAGCTCTGCCTCCCGGATTCACGCCATTCTCCTGCCTCAGCCTCTCGAGTAG
CTGGGACTACAGGCGCCCGCTACCACGCCCAGCTAATTATTTGTATTTTAGTAGAGACCGGGTTTCACCGTGTTAGCCAGGATGGTCTCGATCTCCT
GACCTCGTGATCCACCCGCCTCGGCGTCCCAAAGTGCTAGGATTACAGGCGTGAGCCACCGCACCCGGCCGCTTGTGTGTGTTTTCTACCTCTTCTA
GATCTAACATGTTATGATTGATATAAACAACAGGAGCGACTGACAAAGTTACCATGTGATCTTGGAATTTCAACATGCCTGTCTTGCTTAGCTGGGA
GTTTCCACAGGGTCAGGGAATGACTCCTCTTGGGCCACCAGTGCCCAGAAGTATGCCTAGAACTGCTCTGTCTGAAGGGACAGGTCCCTCTTCCATG
GCACACACTCTGTGTGTAACAGAGCACACGTGTGACCAAACTGACCTGGAGAAAGGGCCTGACCACATCCATATCCCAAGTTCTGAGGCCGGATAGT
GCCTGCAGGGGCTGAGACCAGGGGAGAGGCTCAAGCCAGCCTTCTCTGGTGGACCCTGACTCAGTCCTCTGTGCAGGGCAGCTGCGCCTCCCAGAAG
GCAGAGGCCTCTTGGAGAGTGAGCCTTGCGGTCACAGCGCCTTTCGGGGGCTCCCGCCACTTGCCCTCAGTGGCGGCTGAGACCTGGGGGAGTCCAG
GCAAATCCCTGAGGAGGCCCCACATGCAAGGCCTGAAGTGTCTGGATGGTGTGGCCGGCACCCTTACCCTCCAGACCAGCAAACGAAGCCCAGGCAG
GGAGAGGGGCCTTGTAGTTTTCTGTGGCTGCTGTAAGGAATTTTCACACACTCAGCAGCTTAAAACAACCAACATTTCTCTCATAGTTCCAGAGGCC
AGCAGTGTCTGCAGTGCCCGCTCTCTCCCAGCTTCTGGTGGTTTCGGGCGACCTTCGGCTCTTCTCAGCTGGTCCATGTGCA
HUMAN SEQUENCE—mRNA
GCAGTTGCAGCGAGCAGCAGAGTCCGCACGCTCCGGCGAGGGGCAGAAGAGCGCGAGGGAGCGCGGGGCAGCAGAAGCGAGAGCCGAGCGCGGACCC
AGCCAGGACCCACAGCCCTCCCCAGCTGCCCAGGAAGAGCCCCAGCCATGGAACACCAGCTCCTGTGCTGCGAAGTGGAAACCATCCGCCGCGCGTA
CCCCGATGCCAACCTCCTCAACGACCGGGTGCTGCGGGCCATGCTGAAGGCGGAGGAGACCTGCGCGCCCTCGGTGTCCTACTTCAAATGTGTGCAG
AAGGAGGTCCTGCCGTCCATGCGGAAGATCGTCGCCACCTGGATGCTGGAGGTCTGCGAGGAACAGAAGTGCGAGGAGGAGGTCTTCCCGCTGGCCA
TGAACTACCTGGACCGCTTCCTGTCGCTGGAGCCCGTGAAAAAGAGCCGCCTGCAGCTGCTGGGGGCCACTTGCATGTTCGTGGCCTCTAAGATGAA
GGAGACCATCCCCCTGACGGCCGAGAAGCTGTGCATCTACACCGACGGCTCCATCCGGCCCGAGGAGCTGCTGCAAATGGAGCTGCTCCTGGTGAAC
AAGCTCAAGTGGAACCTTGGCCGCAATGACCCCGCACGATTTCATTGACACTTCCTCTCCAAAATGCCAGAGGCCGAGGAGAACAAACAGATCATCC
GCAAACACGCGCAGACCTTCGTTGCCTCTTGTGCCACAGATGTGAAGTTCATTTCCAATCCGCCCTCCATGGTGGCAGCGGGGAGCGTGGTGGCCGC
AGTGCAAGGCCTGAACCTGAGGAGCCCCAACAACTTCCTGTCCTACTACCGCCTCACACGCTTCCTCTCCAGAGTGATCAAGTGTGACCCAGACTGC
CTCCGGGCCTGCCAGGAGCAGATCGAAGCCCTGCTGGAGTCAAGCCTGCGCCAGGCCCAGCAGAACATGGACCCCTGGGCCGCCGAGGAGGAGGAAG
AGGAGGAGGAGGAGGTGGACCTGGCTTGCACACCCACCGACGTGCGGGACGTGGACATCTGAGGGGCCCAGGCAGGCGGGCGCCACCGCCACCCGCA
GCGAGGGCGGAGCCGGCCCCAGGTGCTCCACATGACAGTCCCTCCTCTCCGGAGCATTTTGATACCAGAAGGGAAAGCTTCATTCTCCTTGTTGTTG
GTTGTTTTTTTCCTTTGCTCTTTCCCCCTTCCATCTCTGACTTAAGCAAAAGAAAAAGATTACCCAAAAACTGTCTTTAAAAGAGAGAGAGAGAAAA
AAAAAAAAAAAAAAAAAAAAAAAAAAAAAAAAAAAAAAAAAAAAAAAAAAAAAAAAAAAAAAAAA
HUMAN SEQUENCE—CODING
ATGGAACACCAGCTCCTGTGCTGCGAAGTGGAAACCATCCGCCGCGCGTACCCCGATGCCAACCTCCTCAACGACCGGGTGCTGCGGGCCATGCTGA
AGGCGGAGGAGACCTGCGCGCCCTCGGTGTCCTACTTCAAATGTGTGCAGAAGGAGGTCCTGCCGTCCATGCGGAAGATCCTCGCCACCTGGATGCT
GGAGGTCTGCGAGGAACAGAAGTGCGAGGAGGAGGTCTTCCCGCTGGCCATGAACTACCTGGACCGCTTCCTGTCGCTGGAGCCCGTGAAAAAGAGC
CGCCTGCAGCTGCTGGGGCCACTTGCATGTTCGTGGCCTCTAAGATGAAGGAGACCATCCCCCCTGACGGCCGAGAAGCTGTGCATCTACACCGACG
GCTCCATCCGGCCCGAGGAGCTGCTGCAAATGGAGCTGCTCCTGGTGAACAAGCTCAAGTGGAACCTGGCCGCAATGACCCCGCACGATTTCATTGA
ACACTTCCTCTCCAAAATGCCAGAGGCGGAGGAGAACAAACAGATCATCCGCAAACACGCGCAGACCTTGAGGAGCCCAACAACTTCCTGTCCTACT
TTCATTTCCAATCCGCCCTCCATGGTGGCAGCGGGGAGCGTGGTGGCCGCAGTGCATGGCCCTGTCCTGAGGGAGCCCTTCTTCCTGTCCTCCTACT
ACCGCCTCACACGCTTCCTCTCCAGAGTGATCAAGTGTGACCCAGACTGCCTCCGGGCCTGCCAGGAGCAGATCGAAGCCCTGCTGGAGTCAAGCCT
GCGCCAGGCCCAGCAGAACATGGACCCCAAGGCCGCCGAGGAGGAGGAAGAGGAGGAGGAGGAGGTGGACCTGGCTTGCACACCCACCGACGTGCGG
GACGTGGACATCTGA

TABLE VI

MOUSE NOMENCLATURE
ICSGNM CCnd3
Celera mCG15576
HUMAN NOMENCLATURE
HGNC CCNDG
Celera TCG1668G
MOUSE SEQUENCE—GENOMIC
NNNNNNNNNNNNNNNNNNNNNNNNNNNNNNNNNNNNNNNNNNNNNNNNNNNNNNNNNNNNNNNNNNNNNNNTGCTTCTGCCTCCCGAGGGCTGGGAT
AAAGGCGTATACTACCACATACATCCCAAAATACATATAAAAATTAATAGAGCTAGTTGAGAAGTCTCTCCTCTCGTGATAGTCTCTGATTTGGGGA
TACACCTTTCTTCATATTTGTATCAACCTATTTGCTGCAGTGTAGGTGAGGCTAGCCCAGTGTGTAGATGAAGTCTAGCCCAGTGTGTAAAGTAAGA
AGGCTAGCCTGGTGTGTAGGTGAAGGCTAGCCCTGTGTGTAGGTGATAGAATTGAACTTTTACTTGAAGAGTGAAAGTGATTTAAACAATGAGGAAG
CGGTAATGCTTTACTCCTCACAGAAAGTCCAGAGGTAGCCTCAGGACTGGTTCAGTAGCTTAGCAGTGTCACTCAGGCGAGGGATTCATTCAGATAA
TCCACTAGGCCCCTTTCTTCATTGCAGAGATGGCAGAGGTGGCCCCAGCATCATAGTAGAGTGGTTACGGGCATAGCTTTGTGGTAGCATTTTTCTG
TGATCCCTTTGGTTGGGAAGCTGAGGCAAGAACATTGCCAGCCTAGGCTACATGGTGAGAATCTGGTCAGGCAGGGCACACAGCAGAGTCCTGTCTG
AGAAGGGTCAAAACAAAACAAAAGCGCAGGGCATATAAAACAGACCTTGTTTGCTAGAGCGTTTGCCTAGTTGCCCACCCTGTAGGAAGCCCTGGGT
TCCCTCCCTAGCAGCTCACGAGCTGAGCATAGTGCTACACACTATAGTCTTCCACCATTCCTGCACTGGGCAGGTAGAGGCAAGAAGATCAGGAGTT
CCAGGCCCAATCAAGTTTAAGTCTAGCCTGAGGTCCATGATTCCCTGCCTCTAAGAGAGATTTGAGGTTATCCTCAACTGTACATCACATGAGAGGC
TAGCTTGGGCTACGTGAGCCACTATCTGAAGAAAAAAAGAGCTAGGAAGATAGCTCAGTGAGATGGTTCAGCAAGTAAAAATGCATGGTGGACAAGC
TTAAAAACCTCTGCTCCGGGGCTGGAGAGATGGCTCAGTGAGTAAGAGCACTGACTGUTCTTCCGAAGGTCCTGAGTTCAAATCCCAGCAACCACAT
GGTGGCTCACAACCACCCGTAATGAGATCTGACGCCCTCTTCCACACAATAAATAAATCTTTACAAATAAACAAACAAACAAAAACCCCTCTGCTTG
GTCCTGGATCCTACTGTGGAAGATGAGGGACACACCAGGAGGTTGTCCACAGGTGCAAACACACACATATGCACACCCAATGTAATTGGAACATTTC
AAATATATCTGAGCATGGTGGTAAAGTGCCTTTAATCCCAGCACTCAGGAGGCAAAGGCGGGGCGATCTGTGTGAGTTTGAGGCTAGCCTGGTCTAC
ATAGTGAGTTCCAGGACAGCCAGGGCTACACAGCAGTGAGGCCCTGTCTACAAATAAATAGACAGACAGATAAATAAATAAGCGATAAAGTATAACA
GATCAGTTACACAGTAAGGCGTATAACAGATCCAGTAGTGTTACACAGTAAGGCCTGACTGATAGATAGACCGGCATACAGAATGATAAAATATAAT
GGGGCTTGTAAGATGACACAGCAGGTAGAGGTGCTTGCTGCCAAGATCTTCAGAACCTATGTAATGAAAAAACAGAACTAACTCTCTCAAGTTGTCT
GCTGACTTCTACGTACATATGCATGGCATACACACGCATGCGGAAACACATACACATTTAAAATGAAATAAACCAGTTATAATGGGGAGCTAGCTAT
AGCTCAGTAGGTAGAGTGCCTGTGTAACCTGTAGGATACCCTGGGTTCAGTCCACAGCATTGTATAAACTGGTCAAAAGATGATCATGGTTGTAATC
CTCGAATTCAGGAGATGGAGTTAAAGAGAAGTTGGATGCCGTCTTCATCTACAAAGTGAGTAAGAGGCTAGCCTGGGCTGCATGAGACCTTATCTGA
AATATAGTAATGGATATTGCTATCTCAGTTTTGCAGTAGGCACAAGGTTGTATATTCUTGCCGTCTCAGAGCCTCAGAGACAGAAACAGGATGGACA
TAAGTTTAGACCAGACGTATTCACTGTGAATTCTAGGCCAGGTAGACCTAGCAAGAGTTGCCTTAGAAACATCAACAAAACAGCAGCAGCAGCTTTT
CATGAAACAGATTCCAGTCTGATGTACACACACATATCAAACCGAGCGGCTATTTGTTCAATTCTTTTTTGTTGTTGTTGTTGTTGTTGTTTGTTTT
TTGTTTTTTTGAGACAGGGTTTCTCTGTGTAGCCCTGGCTGACCTGGAACACACTTTGTAGACCAGGTAGGCCTCGAACTCAGAAATCCACCTGCCT
CTGCCTCCCAAGTGCTGGGATTAAAGGCGTGCACCACCACGCCCGGCTCTATTTGTTCAATTCTTGAGCATGAGGCTAGCTCTATCAGGAGAGTTCT
GACCTAGAGGGGCATGGGACTTGTCATTTCAGAGTGTGGCAGCCTCCTTCTGAGTACCAGGTACCTGGATTGCTTAGTGGCTTGTTCTATCGGAAGC
TATTGTACCTGCCCACAGCAAGGTGGAAGTGTCTAAGAACTGAACCGATCCTGAGTCTGCCTACAGAAGCCTTCAACCAATGGCAGCTGAGAATGTG
GAGACAAACATCTTGCCTCATTGTGCCTCAGCTACAATCATGATACAGACAGTTGTAAGCTGCCATGTGGTTGTGGGAATCGAACCTGGGTCCTCTG
GAAGAGTAGTCAGTGCTCTTAACTGCTGAGCCATCTCTCGAGCCCTTGCCCAGCCTTTTTTCTTAAAATTGTATTTTTAAAATTTATTTTCTGTACA
CAGGTGTGTGAGTGTGAACATGTGGATCCTAGGGGTTGAAATCAATTTGCCGGGGTTTGTATGGTGCCTTCTCACCAGCTTTAGAGTTTCTTGATTA
CTAGCGAAACCTTCCTGGAAGCAGGATGCTACTCAGTATTTTGATCACTCATAAGACATTTCATTGTGAAGTGGAAAGAAAACCTTCGTATGTTGTT
TTGTCTTGTTTGTTTAATATTATATTCTACAGAACATTATATACAGATGTCATAAGGACAATAAGGACATAGTTTATTATTTATTTACTTATTTTTG
AGAAAGGGTCTCACTAAGTAGCCCTGCCTGGCCTGGAACTCACTATGTAGTCTAGGTTGGACTTGAACTCACAGAGAGCCACCCACCTCTGCTTCTG
GAATGCAGGGACTAAAGCCATGAGCTACCATGCCTAGCCAGATTGTAGTTTTGTTTGTTTTTGTTTTGTTTTTCAAGACAGGGTTTCTCTGGGTAGC
TTTGGCTACCCTAGAACTCACTCTGTAGCCCAGGCTAGCCCCAAACTCACAGAGGTCCATCCATCTCTGCCTCCTGAGTGCTGGGATTAAAGGCATG
CATCATCACACCAGCTGAGTCTTTTTTTGTTAGGAAAATCTCCAAGTTGTCTATCCTTGAGCTTTGTGGTATTCTTTTCCTGAGACTGGGTCTCATG
TAGCCCAGGCTGGCTTCAAACTCAGCGGATAGCTGAAGATGCTCTTGAGCTTAAAACTCAGCTTCTTTATTTTCTTTATTTACTTACTTTTATTTTG
AGGCAGAGTTTCTCTGGTGTAGCCCTGGCTGACCTAGCACTCTATTTGTAGCCCAGGCTGGCCTTGATCTCACAGAGATCTGCCTGCCTCTGTCTCC
TGAGTGCTGGGCTGAAAGGTGTGTACTGTTGCACCAGGCTAGCGGCTTTTTTTTTTAAAAAAAAGATTTATTTTATTTATGTATATGAGCACATTGT
TGCTCACACACCAGAAGAGGGCATCAGATCCAGTTATAGTCTGCTGTGAGCCACCATGTGGTTGCTGGGAATTGAACTCATGACCTCTAGAAGAGCA
GACAGTGCTCTTAACCATTGAGCCATCTCTCTTCAGCATGGCCTTTTTTTCTTTTCCTCTTTCTTTTTCTTTTCTTTTCTTTTCTTTTCTTTTTCTT
TTTCTTTTTCTTTTCTTTTTAAAGGCTTAATAACTATTCTTTTTTTTCTTTTCTTTTGGAGACAAGGTCTCATTTTTAGCTTAAGTTGGCCAGGAAT
TCACTCCATAGCCTAGGCTGACCTTGAACTTGGGGAGATTCTCAAATTGGGATTCCAGGTCTGTGCTACCAGGCCCAGCTTATACAATTGTGTTTAT
TTCTCAATTTAAAAAAAATCCATTTTTATTTTATGCGCTGGGGTATTTTGCCTGCATGTATTTATGTGTACACGCATGCCTGGTGCCTGCGGAGGCC
AGAATGAGCCTCAGAGCCCCTAGAAATAAAGTTACAAATAGCTGCACAGGTGCTGGGAAGTGAACCTGGGGCCTCTGCAGATAGCAGCAACTGCTTT
TAATCGCAGAACACTCTTTCTAGCCTTTAATTTCTCAATTGAGGTAAATGTTATTAAAAGTTATAAAAATGGGCCATAAAGGGTATGTGTTCTTGGC
ATACAAGAAGCCCAGGGTTCAGCCTCAGCCACTATTATTATGTTGATAGTTGTAAATACCCATACACGAGGACAAATTTGGCTTTTACAAAGCCAAG
CATAATCAGTTGGACAGTGGTGGCGCATGCCTTTAATCCCAGCACTTGGGAGGCAGAGGCAGGCAGATTTTTGAGTTCGAGGCCAGCCTTGGTCTAC
AGAGTGAGTTCCAGGAAAGCCACGGACTACACAGAGAAACCCTGTCTCAAGAACAAACAAACAAACATCCATAATCACAACTCAACTACCTGGCACC
CACCCCCCTGCATCCCTGCCTCCCCTGCCCCTCCACACACACACTGTGGCTCTGGGGATTGAACCTAGGGACTAAATGAATTCCGAATAAGTGCTTT
CCCAGTGAGCTTACCTCCAGCTCAGAGAAGAGTGTGTGAGAGAGTGGAGAGGGTAATTGTTAGTATATAAGTAACCTTTTTGCTTTTGCTTTTTTTT
TTTTTTTTTTTGAGGCCGAATCTCACTATTCAGTAGTCCAGGCTGTCCTTGAACCTGCAGTTCCTCCTGCCTCAGGAGGATGAATGAATTACGATTA
CAGGTGTGGGCTACCACAATTGGTTTAGAAACTACCACATTTGCATGTTCCCTTTGCCCCGGATACCGTGCAAGATACTTTAGGCCCTTTGGTCATC
AAAACATCCCGGTAGGGTACGGACTTTGTTATTACCCATCTGGATGATGATAAAACAGACACGAGAGAACCAGTAGGTTCTCAAATCACTCCAGGAG
TGGCCTCATTCCGGTGTTGTGTCCACCTTCCTATTTAGGGGTGGTATCTCCTCATCTTTTCCCCTACCTGGTTTCTTCTCTCTCTCTCTCTCTCTCT
CTCTCTCTCTCTCTCTCTCTCTCTCTCTCACACCTCACACACACACACACACACACACACACACACACACACACACACACAGTGGGTCTGAGAGTTA
GGGTGAGGTGAAGGCCCTCACCTGTCACTCTGTTCCCCTTCTGGGTCTCAGTTGGAGAGATTCCTTGATATAAGAGGTGAGGGAGTGGTAGGAGGAA
GGACCGCTCAGAGTCCTTCATCCTCTGGCCTTTCCTTAGGGCTTGTGACAATTACATTCCAGCTGTCCTCTAATTTACCATTTCATTGAGTAACTTG
TAATTGGATGCACCTGGCTTGAAGCACTTTGATTTATTCCCCCTGGTTATGGGGCTAGTGTGAGCTGTCTGGCTCTTCTTTCCCTGTCTCTTTGTCT
CTCTCCTCCTCCACTCCCCTCTCCTCCTCCTCCUCCTCCTCCTCCTCCTTCTTCTTCTTTCTTATTCTCTCTTCTCTCTCTCTCTCTCTCTCTCTCT
CCCACCGCTCTTGCTTGTTTGTTTGTTTTTTGGTAGTTGTTTGTTTTGTTGTTGTTGTTTTGTTTTTGAGGCAGTCTCACCTAGCCCAGGCTGGCCC
TGAATGGCTGCCCACCTTGCTTCCACCTTCTAAGTGTTGAGATTCCCAGTGTGTGTGTGACTCCTCCAAGTTTTGCCCAGTTTTTTATGCAGTGCTG
GGATTGAACTTAGAGCTTCTTGCATCTTAGCCTAACACTACCAACTGAGCCTCATTCTCAGCCCCTTTTTTTGTTGCTTTGTTCTGTTCTTCGAAGG
GCAGTGATGGTGAAGCCGGATAAGCAGACCTCTCTGGAGTTCTCCCGGGGAGGCCTCCTGCTCCCCCAGCAACCACCCTTCATGTAGCTGGCCAGCT
CCTTGGCCCTGAAGGCCCAGGACAGTGGGGCTGTTGACCAGTTATCCAAGAAACCTTCTAGGACTCAACTGTCTCTCTCTCTGGGTGCCCTTTCTCA
GTAGGTCAATGAGGAATAGATCATATACATCCTTGGGACAGCACAAGAGGCAATGGAATGAAGTGGTCAAGAGTTGGTAGTTGACTGCTGCTTTCAG
ATCTCTGTGGCTCAGCACCGCCCCTGCGCCTTTGAGTTCTAACTCAGTGTGCTTAATTTTTTGTTTCCTTCATTCTGTCTTCCAAATTCTTGAGCAT
GTTTGCCCCGGCACTCTGGGATTTCACCCTGTTTGTTATTCTGAAGACACTCTCATCCTTTTCTATTCCCATCGGTGCATTTTTCCATTTATCGGTG
TTAGATTGTGAATGTCAGCTCAGAAGNNNNNNNNNNNNNNNNNNNNNNNNNNNNCCTTTCTTTATATAAGGATGATGACGTAATCACAGGTAAAGAT
AGGAGCTGAACCAGTGGATTCTGGGAATATGGTGGCTGTTGTCTTGGCAACAGGTGTTTGGGGTCCTGCCTATGTGATGTCACGGTTTTTTGGAGGT
CCTGATACCAACAGTCTCTCTCTCTCTCTCTCTCTCTCTGTCTCTCTCCCCCCGCCTCTTTCCGCCACCACCTTGCCACTCTCTCTCCTAATAAACC
TCAGGTGGAACTGTTTGGCCTGGTGCAGTCCTTCTGAAGCTGACATTCACCAAACAGTTGGCAACAGGAGGTCCTGATACTAACACAAAAGGCAAGG
CTTTCTCTCCTCCTCAGTTCATCTCTCTTCCTGGAAGCTTTCTTCCCTGTTTGTTTGTTTGTTTCCCAAACTCTAATAAAATGGTCTTCAAGCTTAT
TAAAACAAAAAGAAGCCATGGGGGTTAGAAAGGTAGCTCAATCGTAGAGCGTACCTACCTAATATGACCAGCATCGATTTCTCCTGATGTCCACCCA
TTGCAAGGGCATCAACTCTGGAGCTAAACCAGACTGCAGCCTGCTTTTACCAACTCCGAGCTATGTGACCTCTCCGTGGTCACTTGCCATCCATGGT
GAGTATTAGCACCCAGCCCCTATTACACAGGGCAAAAATAATATACTTTCTGTAAGGCTGTTGTGAAGATAAAATGATTTATTACCTCTCAGATATG
TTCAGTTCTTGGACATGGTATGTGTACATGAAGACCTGTTACTGTTGTTGGTACTCACAGCCAGTTGTTGTGTCCAGAGGATACACAGCTACGCGGG
ACTTTATCTCTGCCCTTCAGGACAGGAAGTTTGGTGGTGCATTCCTGTAATCTCAATATTTGAGAGGAAGAGGCAGTAGGATTGTTGCAAGCTGGGG
GCTATTCTGGTCTACCTAGAGTCAGAGCTACATAGAGTCAGGTCTATAAATGTCAGAGCTACATAGTGAGAACCCGTGTCATAAAACTAACAGAATG
TTAGTGTGGGTAAGGCATGCAATTGGGAGGTAAAGCAGGAAGATCAGATCCAGCCTCAGTTATATACTAAATTTCAAAGCTAGCCTGGGTGCCGTAA
GACCCTTGTCTCAAAAAGTGTCCCCTCCCTCATTCGTTAAGTGTAAAGCTTGATGTATAAACATGGTGGTACGAATTTGTAATCCCAGCACTTGGGA
AACTGAGGCACGAAGCTGGAGAATTCGAGGTCAGCTATAAAATAAGACAAACTGTTTCAACGAGGAGAGAAACCACTGGGGCAGTGACCTTCTGTGA
CTGAAGCAATCCCATCATGCCTCCTTCCTGTATGAAAAGCCATCCCCATATGTGGATTCTGACCCCTTGCTCAGCGCAAGACTAGTGTGTAGACCAG
TGTGTAGACTAGTGTGTAGACCAGTGCATAGTCCAGTGTGTAGACTAGTGTGTAGATGGATCTCACTGCTTATATATTGTCCTGTGCTTTCCTGGGG
CTCACTTGGGCTGAGCAGAGAGAGCAGTGTCTTAGTGGTGGGCTTTCTTCTTTCTTTCCCTCCTTTCCTCTTTCCTGCCTTCCTTATTACATCCTTT
TTTCCCTTGATTTTTGTTTTTGAGACATAGTCTCATGATATTAGCTCCTCCTGACCTAGAACTCTTATTTAGACTAGGCTGGCTTTGAACTCAAAGA
AATCTGCTTGCCTCTGTCTCAAAGTGCTGGGATTAAAGGGGTGCTCTGTCATGCCTGTCTTCCTTCCTTCCTTCCTTCCTTTCCTTCCTTCCTTCCT
CTTATATAGCCCAGGCTGCTCCTGAACTCATTAGGAAGCAGACAGTGACCTTAATCTTCTGATCGTTTCTGGGTTCTGCAGTGCTGGGGATTGAGCC
CTGGGCTCTGAGCATGCTAAGCAAGAGCTCTTCCACCTGAGATATTTCTGCAGCCCACCCCCTGCCACCGTGTGTGTGTGTGTGTGTGTGTGTGTGT
GTGTGTGTGTGTGTGTGTGTGTGGAGAGAGAGAGAGAGAGAGAGAGAGAGACTCTAGGCAGGCCTTGAATTGTCTGTATAGTGGATAGTGGATCCTG
GTGTTAAGCCAACACTTCCCTGGGTCTGAGATTACTGGCTTGCTCCACCACAGCCAGCACCAAATGGCTTTTTATATGCTGATTTTCACCAACACTG
CTCACACCTCCGTTGGTGCAACGCAGTGTCCCATTCAGCAGACCTTGAGAAACTGCATTTCTTCTTTTGCTTTTATTTTCGTTGTGTATGTGCATGA
AGGTGTGTGCGCACATGCTACATACAGCCTTGGTGTAGAAGTCAGAGGACAGCTTATGGGAGTTGCTCTCTTCTCTTCCATCATGTGGGTCCCAGGG
ATCACACTCAGGTTGCCAGACTTGATGCCAGGATAGTTTACCCTCTGAGCCATTGAATCGGACAGAAACTACATGCTCCCACATGCTGCTGCAGATC
TGCGCAATTCGTTAAAGTAGACGAGATTCTAAGTCAAGTTTCCCGACGGACCAACAATGAGATCTTTGCAGCTACTATTGTGCCTCAGTATCATGGT
CTGCAAAATAAAGCCCAACTCGAGGCCATTCCTAGAAGCCACTTTAGGGTAGGAGTGACCCAACTATGTACAAAGTTATGACCACATTCCCTAGAGC
AATGAAATATGACGTATACACGAAGGAATAACTTTTGGCGCCTAGTTCTTTCCTGGGTGGCTAGAATCGCGACGCGGCCCAGTGATGGGGAAGAGGG
CACCCTTCCAGAAAGTTCTCTCTACCCTCTTGGTCAGAAGGGACCCGAGGGCCCGGAAGCGTGCGGGCCGGAGCCCAGCGGACCAATGGGGCTGCGG
GGAGGGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNCCATCGCAGGATGGAGCTGCTGTGTTGCCAGGGCACCCGGCACGCGCCCCGGGCCGGGCCCGACCGCGGCTGCTTGGGGACCAGCGT
GTCCTGCAGAGTTTACTCCGCCTGGAGGAGCGCTACGTGCCGCGAGCCTCCTACTTCCAGTGCGTGCAAAAGGAGATCAAGCCGCACATGCGGAAGA
TGCTGGCATACTGGATGCTGGAGGTAAGGCCCGAACGGGTCCCCTCTCCTCACCCCACCCCCACCCCCGGCTCTGGGACAAATTGAACTTTCCCTGT
CGGATCTTTGCGATCCCGACCACAATCTGCAAGATGCTTTGGCGGCCCAGACTACTGGGGCCTGGCCTTTTCACACAGGCGCCCTAAGCAGCTGCCT
GCGTTCTGTCCCTTTCCTTTCCCCAATCCTGATCGGCTTCTTCTGTGATTTCCAGTCACTTGGCACTGTAACTAAACTTCACTTTGGGTCGGGAAAG
CGGGCTAAGGGCCACTCTTTCCCCAGTGGCTTTCCTCGGTGTCTCCTGCTTCCTTCTTCACCCGGTGCTCTCTGGAGGGCGCGTTTGGAAAACCCTG
GCCTCGGCCGCCTTAGCCCCAGAACTGGTTCCTGTGTCTATCCCGGGGTTCTGGCGGGGGAAACTGGTGGGTGGGGGTGGCGCAGGCTCGGTTCCGG
TCCACAGGGAATGAAAAGTGCGGGAGACCGCGGCAGGAGTTCCCTCCCTTCCCCCTTGGGAGTGTGTGTGCAGTGGGTGGCGGTAATGAGGGTAAGG
TCCCAATTCTCTCTAGGTGACCCTTCCGTCCCCCTTCCGCTCGCCGTGGGGGAGGGGAGTCTGGAATCCTTCGTGAGCATGGGCTTCTCTTTTACAC
CCCCTACTCCGGGGTGGGGAAGAGACCATGCAGAGGAAGTGGGGGGGTCACCTTGGGGAGGGCACATCCTCCTCGGTTATTGATACTTGTCGCTCTA
AGTTTATCCCTTCTCGGTTTATTTTTTGCTTCGCATTCCACTCTCCCTCCACCCCCCACCCCCACTTCTCCACGCTGCCGTGTTTCACTTGGTCCTT
CCCTAGCAATGACTCGTGCGCCCTCCCTCAGGTGTGTGAGGAGCAGCGCTGCGAGGAGGATGTCTTCCCTCTGGCTATGAACTACCTGGATCGCTAC
CTGTCCTGCGTCCCCACCCGAAAGGCGCAATTGCAGCTTCTAGGTACCGTCTGCCTGTTGCTGGCCTCCAAGCTCCGCGAAACCACGCCCCTGACTA
TTGAGAAGCTTTGCATCTATACGGACCAGGCTGTGGCTCCATGGCAGTTCCGGGTGCGTGCGGAAATCCTCCCCCCCCCCCGCCCCTCCACCAAACA
TACGATCGCCCTATGTACTAGTAAAAAGAAAAGCCAAGCTCACTCCCTGACCCACTCCCTCCCCTTCGCCCCCCTGGCTGGTATTTTGGGACTGGCC
TTGTAGTGTGGTCGTCGTGCTTCGGTGCAAGCTTAAAACTAAATGAATCCCTTTCCTCTGGCCGCAGTGGGGTTCCACTCCTAACTTCGCCCCCCCC
CCCCAATAGCAACAAGTCCAATTGCGGGTCTATACACCAGTACTTTGCCTCTGGGTTTACACAGCCACCGTGAGTTATTTATTGGCAGCAGCAGGGC
ATGTCTGCACACCCCCACCGGAGTGGAATGTGCTTCCGTGGGGGGAGGGGGCTGCCTTGCATGAGCCTTTATCCCCGTGTGGACTTTGTGATGGAAA
AGCCTCCCGGAGCTGATTAAAGCCAGGCCCTGCTCGCTGGAAGGAAGCGCTGAGGTGTTTTCATCCCTTTCCCTCCTATCCCCGAAGACACCGCTCT
TTTCTAAACCCTCCCTTTCTTGTCTTCCTTCTGAAAAACTCGAGCAGCTGCTCTGGGCCCGTGGAAGAGTTTCTCCCTTTTGCTGCTCAGCTAAGGC
CAGAGCCGAGACAGACAGGGAGCTGAGCTGCAGCTGCTTCCGATGTGATAATGATGCCGGGGTCCCCTGGCCCCCTCCTCTCAGACTGGGCTTCATA
ACCCTGTGGGGGCTGCTGTCACTCCTGTGGTTTGGGAAGTGGCTCCTTGGGGCCTCTTGCTGGGAACTTGGGCTGGGCTCCTCCAGGCGGGGCAAAA
AAAAAAAAAAAAAAAAAAAAAAAAAATCGGTTGGGTTCTGCCCTGCCCCGGAGGAGTCTAGGGAGGAGGTGGATCTGCTTGGATCTGGAAGGGAGCT
TGAACGTCATCTCCCACAGGGTCTGGGGGAGAGGGTGGGTGAGCTGGCACCTCCCTCCACCTTGTTCTGTGGCTGGCCAAGCCTTCTCTGACCCTGA
GAGGAGTCTCTGGGCCCCATCTCCTAGGGAGGAGGGCAGCAGGCAGACTCCTGTGATTGGCTGGCTGGGAAGCTAGGGTACTGGGGCCCCTGACTGC
TGTGAGTCGGGGGAACTGGATTCCTCTTCTGTCAAGCAGCTCTCACGGAGGATGGGACCTGAGTTGGAATCCGGGAGGCCCCACAGCTCTTTGTGAT
GAATCTAGTTTCCAGAGCCAGGGAGCACGATGTTTTTTGAGGGATTGAGGCTGGGAGGTAGGAGACTTACTCTTTGTGAAGAGTTAAGGGTGGTCTA
CATGGCTGTGTGTGAGGGGAGTTTTTTCAGAATAGGCAAAGCCCTCCTTCTCCTGCTCGAGGCCCTGAGCTGAGAGTTCCTGCGGTCAGAGTTGTGA
GGGACTCCAAGAACTCCAGCAGTCTAAATCCACACAGCAGAGCAGAGGCCCCAGAAAGGAGGGGAAAATCCGACCAGAGCGCCTTGAAGGGCAGGAT
GGGTTGCTGCTGCCAGCTCCTCTGATCTTGGGGTAGGGCTGGGGTTTTTCATGTCATCTGGATACAGGGGAGCGTGCCCACCTAGTGAGGACTCCCA
CACTCCTGGTTGCCATTGGGGTGCCCCTTTAGAGGCTGGATGCTATTTGGTTATTAGGATAGAGCCCAGGGTGCAAGCTTCAAGCTTCCTTGCCTTA
CTTCTGGCTCTAGTCCAGGCTGTTTCTGTGGGGTTCTGTAAGAAGCCTGGGGCTGACACCTAGGTCATGGGACAAAGAAGAGGGACAGCTGTCACTG
GGGGGGTCCTGGAAGATACTGGGTGGGGCTGACATTTCTACCTCAGTGAGGAGTCAAACCCATGAGTTCCTTCCTGAGATAAAACAATGAAGAAATC
CTCCTGTGTGCAACTCGGAAGTGCAGCTCGGGCCAGGGCTAGGAACTTCATATGAGTAGACTGAGGTTGGGGCTCCTAGCCTCCTCCTAAGACCTCT
GAATGGTTATGTAGGCTTGGACAGGTTTCTCAGGGGTCATCTGGATTCATCCTGTTTGGCTTGGATGCCCTTGTCTGGAGACAGGACCCACCCGTGA
TGTCCCACAAAGGGGGGTGTGGGTTGGGTTGGGCAACCTCCTCTTTGAGGAACCTTAGGAGTGGCTTCATGCTCATTTTGTTTAAGAGACCTGGATG
CGATGTTGGGGAAGAAGAGATTTCCGAAGCCTGTGGAATTTAGAGTTGGTCTGTTTCAACTCACGCCCGCCTCAGACTCATGGGAGGATTAAGAGGG
ATTAATTCAGGTCGTTCTAGCACCTAGTAACCATCAATAGATGCTTAAGTGTACGGCCCTAGTTCTTCCCTGCCCCAAACCCGTCATCCCGCGGCAG
CAGCCCCTGAAGCCAGAGGCAGAACTGTGACCCTTCTGTCAAATTCTCGGCCCCCTCGGCCGACTGCTGACTCCATGTTCCTTCCCTAACCCCCAGG
AGTGGGAGGTGCGGTCCTGGGAGCTCAAGTGGGACCTGGCTGCCGTGATTGCGCACGACTTCCTGGCCTTGATTCTGCACCGCCTTTGTCCTCTGCC
CAGTGACCGGCAGGCTTTGGTCAAAAAGCATGCCCAGACCTTTTTGGCCCTCTGTGCTACAGGTAAGAGCCAACCTGGGGGAAATTGTGGGGTTTTT
TGGGAAGGATACGTAGTAACAGCCAGGAGATGGTTTCAGTACTCAGACCTGTGTACAGTGATCTGGGAATACTGAATAAAAGTTACTTGCCTAAGGT
CTTCATAGCAGGGTTTTGGATATTTTGTTGTTCCTGGTGCCAGGTACACCTTGGGTAGATGGAAAGTTTGTTGTTTGCTCATATCTCAGAGGAGGTC
GGGCTGTGGTCTGGTGGGTCAGTGATAGGGTGCATTCCTCAGTCAGATTGCCAACACTGTGAGCACACACCAACCAAACCCCCTCCTCCCTAGGATC
TACCTTGTCCCCTTGGCTCAGTGTCACCCCATTCACCACTGGGGGTTCCCAGGATGCAGCCTGGTGTGTTGTAAGATGGCTCCCTTTCTTTTCCCAG
ATTACACCTTTGCGATGTATCCTCCATCCATGATCGCCACAGGCAGCATTGGGGCAGCCGTGCTAGGCCTAGGCGCCTGCTCTATGTCTGCGGATGA
GCTCACAGAGTTGCTGGCCGGGATCACAGGCACTGAAGTGGTAAGTCTCAGCCCCTCGCCCCCCTTCACAAAATTAAAAAAAAAAAAAAAAAAGCTG
TTCCTCTGAATCTCCAGCAGAGGGCACCATCCACCCGGGCCCCCAACTATGATTGTACGCCCTTGCCTGCTTCTCTGGCCTTTTTTCTCTGGCTCCA
GTCTGGAGAGGAGTGGGTGGGGTGAAGGACACTTGGCATGTAGATACCCTTCTCTTGACATCTCTTGACAAGTGCTGTGTCTCGGACAGGGGTGGGG
GACTTGGTGTCAGATGGAGGGTTGCTCTCACACCCTTGCCACATCCTCTTGCCAGTTTCTGAGAACTAAAGACCTGATTCCTGGGCTGGGCTGTGGC
CTAACCTCCCCAGACTTCCCAGTGCACAGGGGGTGGCCTTTCCACCTGCTTGCTGTCAGTGCTGTGAGGGAGAGAGAGAGGCCCAAGGATGAATGAC
CTTGTTTTTTTTTTTTTTTCTAGGACTGCCTGCGAGCCTGCCAGGAACAGATCGAAGCTGCCCTCAGGGAGAGCCTCAGGGAAGCTGCTCAGACAGC
CCCCAGCCCAGTGCCCAAAGCCCCCCGGGGCTCTAGCAGCCAGGGGCCCAGTCAGACCAGCACTCCCACAGATGTCACAGCCATTCACCTGTAGCTT
GAGACAGGCCCTCTCAGGCCACCAAGCAGAGGAGGGGCCCCTGCCACCCCCTCCCTGCCTCTAGAACAATCCATGCTATATCTGATGCCCGAGGGGG
GCTCTCCTTCCCCCTCACAAAGCCCAAGGGGCCAGGTCCTGCCTATCCCCACAGTGTGCACTAAGGGGTTGCATGGTCATGAGGGGCGTCTTCATGG
CCAGTCAGCTCCTCTTCCTTCCCACTCAACCAGCTTGGCTGTCCTGGGCCATGATGGTCAGAGAAATACAAACAGGTAAAATCCACAeACCAGCATT
TCTTTTGAGTCCCTCTTCTGTCCGGGGCTCCAACCTTCTCAGTTGCCAAAACGCCCCAGTACCTTCCAAAGGTGTTGGCCCTTGCAGGCTCCTTCGG
GCATCCCGATAGAAGCTTATGAGGAGCTGCCCTAGATGGCCTCTGTGTAATCCGTACTCCAGCTGCTCTTAGAGGGAACAGCCTAGGCTTTGGCCAG
AGCAAGAACCCATACACTGTTGCTTTGCTTGCTGCTTAGCTTCTGTGATTGTGGGGTCTTAAGGGTGCCGTGGTCATTTTAATTTATTGCTTTGAAT
ACAACTGTAAGAGGGTACAGTGAGGCCTGTACCCCACAAGTGGTGGTAACCCTGGCGGTTGCTCTTTCCCTCCCTTCTTCCCCTCTGCTACTGCTTT
GTGGCCCAGGAGCTGCTACAGCCTGGGATGGGGGTCACGCCTTCCTCTCCTCACCCCTCCACCTCATCTTCATCAGAGCAGGGTTAGGTTGGGATGG
ATCGATGCCGTGGAGGTGACAGAGCTATCTGGAGAGAGGGCAAGCCCTAGGGTCACAGGTCTTTCCTCGGGCCACAAGGTTTGGGCTGGTGGCCCAT
TTCTATCATGCTGCCTTAATAAAGATTTCGGAATAAAGGTGGCTCTTGTGATCCCTGGGGTTGTGGGAGCTGGGGGGCACCAGGCAGCTGTCTTAGG
CAGAGCACTTTTCCTGGGAAGGGTTAGAAAGGTGCCGCCTAGCCAGCATGGCCTGCTTTCCTTGTGGACGAATTACAGCGCTAAGGAGCAGCCAGCA
TTACCCAGAGGGCTCATCGGGAGACAGGGCTGGACCCTTACACAGAACCCTAAGATTCATACGACATCAGGCATGGTGCTTCCTTAGCACACTCAGG
GCAGACAGAGGAAGAGGGCTAGCCTGTGCTACATAGTGCAGAACTTTGGCTCAGCGACAGCACTGGCTACTCCTGAGAGCCTGGACTCAATTCCCAG
CAGTTAACCATTTTAAACTCCAGTCTCAGGGATCTGATGCTGAGGCACAAAGCTCAGAATGTATTAAAAAAACAAAACCGGATGAATGGGGTTAGGG
TGTGGCTGGAAGCACTTCCCCTAGCAGGGAGGCAGGCCCTTCCTCAGTGGTGCTGCAAAGGAGCAGCCCTGCATGGGAGAGGAGAGGAGAGGAAGAG
CGAGGGAGGAAGGGAGGGAGAGCTTGAGTGAGCTCAGGGAGTAATTTGGTTGGGTTCGGCTAGGTTTGTTCTCTTTGCTTTGCCCTAATGTTTACAA
GATGATCTACTGTGTAAGCAAATATGAACTCAACAGACCAAACCTTCTCAGATATACAAACTGGATTGTTATAGACACACGTTTTCAAAAGTGAACG
TGATACAGCGAGGTGACTTCAGTCAGTCAGCTGTGATGCATGGTTCTAAGGGTTTTTGTTGTTGTTTTTIGGTGAGGTAGTGTTTCACAGCAGTGGC
TCAAAAAGTTGAACCATCAGGCAGGTGGGTGGCCAAGTTCAAGGTCAGCCTGGTCTACAGAGTGAGTTCCAGGACAGGCAGGGATACACAGGGAAAC
CCTGTCTTAAAAAACCAAAACCAAAACCAAAACCAAACCAGACAGTATTCTTTGCTCATTAGTTATGCAGACACAGGAACCTGACCTGGCGTCTCAG
CCAGCCTAAAGCTCTGGTTGCTTGATAGCTAGGAAGAAATCCCATTTTTCCTGATTCTGTTTGTGTCACTGTCCCCTAGCAAGGGCCTGCAATATAG
TCTTCCCTGACAGTGAACCACTGCCACTTTTCCAACAGACACCTTTCCAACATGGCAGCAGAATCTGGCATCCCTAGAGTCGGCCCTGACTGTACGT
CATGGTAGGAGTCAGGGGCGCGTGTGCATACATTTCCTGTGAGAACAGGAAGCAAGAGTTCTAGAGACACAACTTTGAAGAATAAGGTGGGGGGTGA
GGTTTCTGTGGGAAGTTAGAACCCGCTCTTCTGAGCTGGGGACAGGGAGGAGCCAGCAGTGACTCCCACCTCTCCCGTGGGTGACAGGGGAGAGAGG
CAGGGACCTCCAGGGACCAAAACACAAGAGCTGCGATAAATAAATGCCTGTTTTCTCCGCACCCCAGCGAGCTTTCACAGTATCTGGAGAGAGCAGA
CCTTATACCAACTGCCAGGTGGGTTGTGGGGTCGCTGAACCGAACTGAGGTAACTCCGGTTAGCTTCCAGAAGTGAGCAGGGACCAAAGCCTAAAAT
GGCAGAGAAAGCCAACCAGATGCAGTCCTCCCTGCCCAGGATTAGGCCACGGCAGCCATAGTAGCCCTTCAGTGTCTTGACCTAGGTCTTTAACTAT
ACCAGCTGGGGTCCTGGGGTGGTCCTGCGCCCCCTGGGCCAACACAGGTGACTGTTCTCATTCCATGGTGACACCACCATCCTCCACGTCTCTGGGG
ACAGCGCTCTGAAGCTCACGCCTGATCTCAGGGGACAGCTGCGGGTGGGCTGCAGCCGAAAGCAGCTCCAGGAGGGCCTCCTTCTGCTCTGTGGCCA
TGTCAGCCTTATAGCGCTGCGCTAGTGTCAGCAGGCACTGGTGCCAGAGGACAGGCAGCTGGCGCTTTTCCGTCCGGAAGGCCAGAAAGTTGAAGAC
CAGAGCGTCGAGCACGCGGTAGGGCAATGCGTACTTCTTATCCAGCAGCAGGCGCAGGAAGATGCTGTTGGCACCGCTGTATTCCATTTCTGCGATC
TTCAGCATGGCCGCACTGGAAGGGAAGGTGGAGTCACAGGAATTAGGCCACACCTATGGGAGTCCTCTTCTTACACTGAGCCTTACTTCAGTGTTCC
CAAGGGGGCCACCACAGGCTGCTCAGCTGTTGATTACTATGCTCCTGAGAGTTTCTACCATCTCCATCCATCTGCCCCACCCACACCTCTGCCTCTG
GCTCGGAGAGCAAGCAATCCCTGTGTTTAACTACAGATCATGTCCAAATGTCTCACGATCCGCACATTTTAGCACACTTTCTTCCTAGATGCCCATT
TACACACGCAGTCATTTCAATTATACTAGCTACCAGGACCCTGCCCCTGCTCTTGAAGACCTACTATTCTGTTTTGAGTTTCATACACCCACTGACA
TAAACGGCTAGACTCGAGGTAATCATAGCTCATTCTCCATTCGCACCCTCACGACCAAGTTCCGGCCTCTCTATCACACGGCTCCTCTCTGACCATC
CTCCTCCACCTCCCCACCACTACCTGGAGTGCAGCACGGGATGGAGCACTTGGTGATGATGCTGCCCACGATGGATGGCTTCACGGAGGGTACAGGT
GCCGGACTCACACAGTGGGATCAGGATTCCTAGGCAGAGTCAGATGCAGTGGCTTTTAGACCGGGGTGAGGGTTGAGGCGCACCCCCCCCAGCAGCC
CCAGGCATCAAGGACCCCCAATGATCTGCCAAGTAAGAGCCCCACCCCCTCCTTTCTCCCCTGCTTCTGCTCTACCTTTGAACCAGGCTCCAGGCTT
GAACAGGGCCTTCTTGAGCGCCATGTAGAGGTGAAAGTTGAGTCGTTTGTATTCAGCAATGTCATCTCGCACTCGGGGAAGCAGGACAAGGTTGTAG
AAGCGCTGAGCCATGCGCTCCTTTCAAGTTAGAGGCAAAATCCTAGCAGGGTAGGAGTTTCCAAGTTACCAGAGAGCCTGACCCTGGCCCTTTAAGA
ATCATCGCGGATGGTGCATTCGAGAGGTTTTATAGTCAGACACGGGACAGCGTGTTTTTTATAGTCAGAGCTGTAGCATGGAGTGAAAGCTCTTCCC
ACTGAAGAGTACCACAGGACTTGGTCATGAAACCTGGTTGGCTGGTGAGCTCAGTGGGTAGGAGCACCCGACTGCTTCTGAAGGTCAGGAGTTCAAA
TCCCAGCAACCACATGTGGCTCACAACCATCTGTAACTGATCTGACGCCCTCTTCTGGAGTGTCTGTGACAGCTACAGTGTACTTACATATTAAAAA
AAATAATAAATCTTTAAAAAAAAAAAAAGAAAAACGAAAGAGCAGGGGGCTGGAGAGATGGCTCAGCGGTTAAGAGCACGGCCTGCTGTTCCAGAGG
CCTTGAGTTCAATCCCCAGCAACCAGACAGTGGCTCATAGACAATCTATAATGAGATCTGATGTCCTCTTCTTCCCGTAGGCATATATGTAGACAGA
ATACTGTATAGATAATAAATAAATCCTAAAATAATAATAATAATAATAATAAAATAAAAACCAATAGACCTATAGTTTCCCCTCTAAAAACTAAAAG
CCAAAACCATAAGACTGAAAGTGACTGAGTTCAAGGCCAGCCTGGTCTACAGAGTGAGTTCCAGGACAGCCAGGGCTACACAGAGAAAACCCCTGTC
TCAAAAAAAAACAACAATAACAACAACAACAAAAAAAAGACTGAAAGTGAGAATATGGAAAAATGACATACAAGAAAATGGAATCTAAAAGTCAAAC
TTGAATATGCAGTCACACCTGAAAATAGGTTTAAAATCCCCAGAAAGAACAAAGTTAAGGGAGCAGCAGGAGCGCAGGAGCAAGGAGGCCATCGGGA
ACTGAACAGCTCTCTGGCAGGCAAAGCCCCGCACCCCCCACAGCCCACAACCTGGCCCATCAATGATACTGGTGGCTGATACATGGCGCTGCAGTCC
AGGCCTCAGGCTCAGTGACATAGAGGATCTGCTCCCAGTTGTACAGTGCAGGGGGATGACCTTAATGCCTTGTCAGTTTCCCACTGCGGTACTTACA
TAACACCTGGGGTGAGAGAAGCAAATCGCCAACTGTGCTGGGGTCCTGGCCCCTGTTGCTCACTCATCTCCAGAACCATCTAGAAAGCCTCGTTCTC
CGGGTGACCGGAGTTTAGTAGACTTCCAGGGTCTAAGAGACAGTGCAAGAACCTGAGCCTATCAGCTGTCCCCCCTTCCCCTGTTCTGATCGCCATG
GCTTATTTGGTGGGGGGCCATGGATGAGAGCATAGGGAAGCCCGATACCTCTGACATGACAGTCTCCACCTCTGTCTGCTTCTCCGTAACTCTCCAT
CTTCCAGGACAACCGGGATCCAGCTGAGGCATAGGGAGCCCGATACCTCTGATTGACAGTCTCTCCTCTGTCTGCTTCTCCGTCAAAACTTTCTCTT
GATGATATCAGCCAGGGTGCGCCTATGGAGACACGGGAAAAGAGAGGTGGAAAGAATTGGGGTCACTTTCCATGAAACTGTAACGTCCCCCTCAACC
TGTTTTTTATCTTTTTGTTGTTGTTATTTGAACAGGGGTTTTTATGTCTAGCTTTTGCCTGTCTTATCTGGCTCTGTAGAAACTTCTGACCTCAGAT
CAGAGATCCACTTGTTTCTGCCTCCTTTGTGCTGGGATTAATGGTATGCCTGGCTCTGTTTATTAAAAAAGAAAGGCAGTAGTGGTGGTGTAAGCCT
TTAATCCCAACACTTGGGAGGCAGGCAGATCTTTGTGAGTTGAAAGGTCAGCATGGTCTTCATAGGGAGATCTAGGCCAGACAGCAAGCCCCGTCTC
AGAGGGGAAAAAAAAAGAAAAGTCACAAAACATCCCCCAAACAAACAGTTGCTTCACAAACTTGCATGTCCTCCTTGTTTTGCCACGTCCCTGTCAC
TGTTCTAATTTTAGTGTACGGAAGCTGGAAGCGAGCAATGTATAATTCCCCTGGACGCTGACAAATCTCGATCCCCGCTGGGATGCACAGCCTCAGT
TCCCCTCTATGTCCATTTAGCCGCCTCAAAGTGCTGCAGAGCACGGGCCTGGATGTCTCTGCATCTGACGCAGAGGCCAGAGTGGTGGGGACGTTAG
GCCCTTAGGCCTGAAGTCAGCTCCGGCTCTTCTGGTCCTTGTATGATCCTGGGCCCGTCACTTGACTCCTCTAAGCCTCAGTTTTCCTCATCTGTAA
ACTGGGCAAGTAAAAAGGACCCCCCCTGGACAGTCAGCTGAAGGGTAAAATTAAGACTGCAGCATCCCGACTCTCAGCCAGAGCCAGCAGGTCCTGA
GGGCTGTTTACTTCAAGCTCACCTCCCACTGCTCACACAACGGGGAGCAACTTCTGAGTGCAGAGTGGAGCTCTGTGCCAGGCAGCGCTGGTGCACA
CCTTTCATCCCAGCGCTCAGAAGGCAGCGGCAGGCAGAGTTTTGTGAGTTCAAGGCCAGCCTATCCTACTCACCCAGCACTTGCCTAGCAAGGATGA
GACCTGGGTTCTGGCTTCCAGCACAAGGGGAAAAGTTTCTCTGCAGTCTTTTTTTCTGTGCCGAGCAGCACTGTTTCTGACACTTTTCTGACTTGTT
CCTGTGGTCTGCATGACACTGGTGGCAGGTGCTTTTTATTCCCCCCGCACTGCCCAGGCCGGGAATCGAGACTCAGGGGCCAACCAACATCTCCTAA
GCCCTAAGACTTTCTCAGGGAAAGGGTAAATTTTTTAGTCTTTCGACTTCTAGTTTTTGCTTCGCAGCGTCTTCTTGTGTCCCTTTGTGACAAGGCC
TGATTGCTGTGTTCCCAAGACCCCTTAGCCTGGTTTCTGAAGATGCCAGTGCCACTGCTTTGAGTGCCCCACGGGTCCCCCCTTTCCACTTGTTGTC
ACTTCCGTATAGCAACTGCAGAAACATCTTCTGCTAAGTTTGTCAGATCAGAGCCACTGTTATGAGGTGCTGCCCGAGAGCTTTTTGGGGGTCACAT
CACACCCCATGACCATTCTCAGAGCTGTCAGACACCTTCAGGGGGACAGGTTCTCCTGTGTTCTCTGAACCCAAGCTGCTCATCACCGTACTATTAA
TGATAGTGGTGTACTACACCCCACTCTGAAGAATAAACTGGTTGAGCCCCCTTTGCCCTAAGCCCAGTCCTCTCAAGAGGCCAGATCCATCTGAATG
GAACCAAGAATCTGACCACAGGCAGCCTAAATAACTGCATGTTTAGAAGTAGCATTTATAACACATGGGGGTACGTTTTGGTTCCTACCCAGAGGTA
GTTTTTAACCCCTCTGGGAACATTTTTGTCAGAGATAGCTGCTATTGGCCCCCACACTGTAACTGAGATGGTTCCCTGAGTTTAGACTAAAGCTATG
GGCTGGCGAGATGGCTCAGCAGGTAAGAGCACTGACTGCTCTTCCGAAGATGCTGGGTTCAAATCCCAGCAGCCACATGGTGGCTCACACCCACCCG
TAATGAGATTTGGTGCCCTCTTCTGGTGCGTCTGAAGCCAGCTACAGTGTACTTATGTATAGTTTTTCTAAATAATGGGCCAGAGCGAGTGGTTTGG
CCAGAATGAGTAGAAGTCCTAAAATTCAATTCCCAACAACCATATGATGGCTCATAACCATTTGTACAGCTACAGTGTACTCACATACATAAATAAA
TTAATCTTAAAAAAAAATAAGACTAAGCTATGAGAGTAACGTACCAAGAATAAACACTACTATCGAAGATCCCCAGCTATCACTGGTCCGGTTAATC
CAGAGGTTCCAGATCAGATGGGCAAAGGGGAATCCGGGTCACTCCCGTTCAGGGGCCACTCTTGGCAATGGTCAGACCAACAGCTGTGGTCTGGATG
AACTTTCTACCAGGACCCACTGGTCCAACCTTTAAGCCTTACCTCACGGGAGGATTCTTGTTCATGAACATTTCAATGGCACGCTCATCTTCAGGGT
CCACGATCACCTCTGCCTGGTGGTCCACCCCGGCCATTTTTGGCTGCCTTCTCCAGGGTAGGCCACTCCTCATCCTCCTCATCTGATCCATCTGTGG
CAATCCCGGCCCTACAAGATAATCCCAGCCGCAGGGGCTTGTGAGTCAGTTCCCCATTCCACGTACAGCACACCCAGGGAGCAGGAGGGACACCTGC
AGAGGGACTATCCTTTTTGTCCCCAGAGACTTTGGAATCTTGATAGAAAATACAGCACACGAGCGAGAGATGATTAGGCCAGTTTGACTTATGGACA
TGTCCCCTCAGTGAATGGATGACATGTCCCCCCTCAATGACCAAGAAGGATCCTTCTATACTACACACATGTTCCTGGTGCTGTCTGATGACCTTTC
AAGGAATAGGTACTGTTGCTATACCAGTTCAAACCGGGGGCAGAAGAGTTGATGTGCCCAGAGTAGGTAGGCACTGTGGGTCCTGACTTCCCACTAT
GTTGCTTGCACAGTGGAACTGCATCATGTGCTGAAAAACTAAAAACCATGGTGGAGGCAGTGTGCACCTTGGCACTTGGATGAGTACAGATCTGATA
GGCTCTGGATGGGCAGTCTGGGAGCAAGAGCATGCTCCACGTCCTGGAAAGTAATGCTGTTAGAGTGTTGGGACTAAGGAGCCAGAAAAGGAAACGG
AAAGCCAACCTGGAGGCTCTAGGCTGGGCAGTGGCATTTCGGACTCTGATCAACCAACACATTTTGTGTACTGGCTTAGGTAGTCTCGATATGACTT
GGGAGTTCCTTGTTCTCGCGCCTCCACTTTCCAAACACGCACCACAGTGCTGAAGGCTGAGCTTCTGATGTTGGGTTAATCCCGTGTGTTCCATGTA
AAGGCTGCCTGTGTACTATTCACATGTTAATCTTTGTTCCAGCAGAGAGCTGAGTACAGTCGGCATTGGTTTTTTTTTTYYYTTTTGCATCACCTGT
CTCAGTGTTGTGGAAACTTCTTCTCCATCACCTTCTGATTGGTTTAATCAAAAAGCTGAGTGGCCCATAGCAGGGCTGGAAAGGATGGCGGGACTTC
TGGGCAGAGATAGGACTCTGGGAAAAAGAAAATCAGATGTGAGGGGGAACTCAACCAGCCAGACACAGAGGTGTCACGTGCTGTTACTTGAGCAGTA
ACGAGCCCCGTGGTTGAATGTAGACTAGTATAAAGCAATTATGTTGTATCAGCGAGTTCGGAACAAGGCAGCTAAGGCCAAGCTTTCATAACTAATA
AGAGGTCTCTGTGTCCTCACTTGGGTGATAGGTGCGTTGAGCCATTCCTGCACATCCTGAGGTCCTGACCATGGAGTCTTCTGTGGCTGTTACTCTG
AGTGCTGGGATTAAGTCATGTGCCAATACACCGGCTGACTTTTTGAATACCGCACATAGCTGGCAAGGCAGTTGGGAAAAGAGTCTAATAAAACCAA
AGTAGTTGAATTATTACCATAGCAACAGATCAGTTGCTATGGAAGTGGACTAAGGGTATATTATATAGAGCTCCAGAGAGTACACAGAGATTACCCA
GTGAGACCAAGGTTGTTTGTTTTAGGTTTTATTTTACTTTTAATGTTTGAGTGCTGACTGCACGTATGTCTGTGCACAAGTGTGCCTGGTGTCTAAT
GACATCAGAAGATGGCATTTTGATCCCTTGCAAATGGAGTTACAGACAGCTATAGCCTGCCATATGAATGCCAGAACCAAACTTGGGTTTTCTGCAA
GAACAAGTGTTCTGAAGAGCTGAGGACCAACTCTTGAGAGTTTTCCTCTGTTCTCTACACACATCATGCCACGTCATGTCACACAAAATACATGTTA
ACAAAGGAAGACAAGAGCTATGAAATACGACGTAGGGAAGAGAGAAAAATCAGGAGACAGTCACTGACATTTGTGTGGTGTTTTTGTTTGTTGTGAT
CATTTGCTTTTTGTATATGAGTGAACCCTGCATCATGGAAGTGACTCTTGGGAGCTGATGACCTTGTTTTTGAAACAGGATTTGACTGGGACCTGGG
GATAGCTGATTAGGCTAGACGTTGCCCAGTAGGCCCAAGAGATCTGTCTTCTACACTGTAGCTCAGAAACTACAAGCTGCTGTGAGTGGTGAAGTCA
GGGCTCATGCTCTCTCAAAGCATGGACCTCACCAGCTGTGAGCATTATCTTATCCTGTCCCTAGCTTGTGGTCAGACCTGAAACAGAGACAGACCAG
ACAGGACGCCATCAATCTCTAGCTGCTGAAATGGACCACAGAGGTCCAGAATTCAAGCACGATGTTTCCACAGTGATCCAGGGGGAGAGACCCTGGT
AGCTTAATCAGGGAGTGTCTCAGGTGAATGGAAGTGGTCTGGCTCTGAAATACTCTAAAGACAACATCACCAGGATTTACTTTGTGTGAGATGAAGA
GGAAGCCTGGACTCTGACTGGAGCAGTGGAGCTAATGTGCTAACAACCTCCAAGTAGCAAACAGAGAGCAAGTTTGGGAAGGAAGTCACAACTTCTG
TTTTAGATGCGATCTTCTTCTTTTATTTTTTTTTTTCCTTCTCCGAGACAGGGCTTCTCTAGGTAGCCTTGGCTGTCCTGGAACTTACTTTGTAGAC
CAGGCTGTCCTTTGAACTCAGATTCCACCTGCCTCTGCCTTGTAGTGCTGGGATTAAAGGCGTGCGCCACCACTGCCCCTGCTTGGACGCAATCTTA
CTAGAGTTAGATACTAGTATTCAACTTGGAGATGTCAGGTAGACCACTGGATATAAAGATGTGAGCTTTCTTGGACGAACTCTGAGGGAGAAAAAAC
TGGAAGTAGCTGTCAAACAAATGGCCTCAAAGTGAAATTCCCTGAC
MOUSE SEQUENCE—mRNA
AGCGCCAGCGCCAGACCCGCCCGCACCCCGCGCTCTCCCTCACGCGCGTCGCTTCTCCTAGGACTCGCTAACCCGCACTCTTGCTCTCACCCGTGAG
CCATCGCAGGATGGAGCTGCTGTGTTGCGAGGGCACCCGGCACGCGCCCCGGGCCGGGCCCGACCCGCGGCTGCTTGGGGACCAGCGTGTCCTGCAG
AGTTTACTCCGCCTGGAGGAGCGCTACGTGCCGCGAGCCTCCTACTTCCAGTGCGTGCAAAAGGAGATCAAGCCGCACATGCGGAAGATGCTGGCAT
ACTGGATGCTGGAGGTGTGTGAGGAGCAGCGCTGCGAGGAGGATGTCTTCCCTCTGGCTATGAACTACCTGGATCGCTACCTGTCCTGCGTCCCCAC
CCGAAAGGCGCAATTGCAGCTTCTAGGTACCGTCTGCCTGTTGCTGGCCTCCAGCTGCGCGAAACCACGCCCCTGACTATTGAGAAGCTTTGCATC
TATACGGACCAGGCTGTGGCTCCATGGCAGTTGCGGGAGTGGGAGGTGCTGGTCCTGGGAAGCTCAAGTGGGACCTGGCTGCCGTGATTGCGCACG
ACTTCCTGGCCTTGATTCTGCACCGCCTGTCTCTGCCCAGTGACCGGCATCTTTTTTCAGCATGCCCAGACCCTTTTTTGGCCCCCTCTGTGCTAC
AGATTACACCTTTTGCGATGTATCCTCCATGATCGCCACAGGCAGCATTGGGGGCAGCCGTGCTAGGCCCTAGGCGCCTGCTCTATGTCTGCGGAT
GAGCTCACAGAGTTGCTGGCCCGGGATCACAGGCACTGAAGTGGACTGCCTGCGAGCCTGCCAGGAACAGATCGAAGCTGCCTCAGGAGAGCCTCA
GGGAAGCTGCTCAGACAGCCCCCAGCCCAGTGCCAAAGCCCCCGGGGCTCTAGCAGCCAGGGGCCCAGTCAGACCAGCACTCCCCACAGATGTCAC
AGCCATTCACCTGTAGCTTGAGACAGGCCCCTCTCAGGCCACCAAGCAGAGGAGGGCCCCTGCCACCCCTCCCTGCCTCTAGAACAATCCATGCTA
TATCTGAAGCCCCGAGGGGGCTCTCCTTCCCCCTCACAAAGCCCAAGGGGCCAGGTCCTGCCATCCCCACAGTGTGCACTAAGGGGTTGCATGGTC
ATGAGGGGCGTCTTCATGGCCAGTCAGTCCTCTTCCTTCCCACTCAACCAGCTTGGCTGTCCTGGGCCATGATGGTCAGAGAAATACAAACAGGTA
AAATCCACACACCAGCATTTCTTTTGAGTCCCTCTTCTGTCCGGGGGCTCCAACCTTCTCAGTTGCAAACGCCCCAGTACCTTCCAAAGGTGTTGG
CCCTTGCAGGCTCCTTCGGGCATCCCGATAGAAGCTTATAGGAGCTGCCCTAGATGGCCTCTGTGTTTCTCCGTACTCCAGCTGCTCTTAGAGTAA
CAGCCTAGGCTTTGGCCAGAGCAGAAACCCATACACTGTTGCTTTGCTTGCTGCTTAGCTTCTGTGATTGTTGGTCTTTTGGGTGCCGTGGTCATT
TTATTTATTGCTTTGAATACAACTGTAAAGAGGGTACAGTGAGGCCTGTACCCCATGTTTTTTTCCCCTGGCGGTTTGCTCTTTCCCTCCCTTTCT
TCCCCTCTGCTACTGCTTTGTGGCCCAGGAGCTGCTACAGCCTGGGATGGGGGTCACGCCTTCCTCTCCTCACCCTCCACCTCATCTTCATCAGAG
CAGGGTTAGGTTGGGATGGATCGATGCCGTGGAGGTGACAGAGCTATCTGGAGAGAGGGCAAGCCCTAGGTCACAGGTCTTTCCTCGGGCCACAAG
GTTTGGGCTGGTGGCCCATTTCTATCATGCTGCCTTTTTGATTTCGGAATAAA
MOUSE SEQUENCE—CODING
ATGGAGCTGCTGTGTTGCGAGGGCACCCGGCACGCGCCCCCGGGCCGGGCCCGACCGCTCTGCTTTGGACTGCGTGTGTCCTGCATGTTTACTCC
GCCTGGAGGAGCGCTACGTGCCGCGAGCCTCCTACTTCAGTGCGTGCAAAAGGAGATCAAGCGCACATGCGGAAGATGCTGGCATACTGGATGCT
GGAGGTGTGTGAGGAGCAGCGCTGCGAGGAGGATGTCTTCCCTCTGGCTATGTCTACCTTATCGCTACCTGTCCTGCGTCCCCACCCGAAAGGCG
CAATTGCAGCTTCTAGGTACCGTCTGCCTGTTGCTGGCCTCCAAGCTGCGCGTCCACGCCCCTGACTATTGAGAAGCTTTGCATCTATACGGACC
AGGCTGTGGCTCCATGGCAGTTGCGGGAGTGGGAGGTGCTGGTCCTGGGGAAGCTCAAGTGGGACCTGGCTGCCGTGATTGCGCACGACTTCCTGGC
CTTGATTCTGCACCGCCTGTCTCTGCCCAGTGACCTCAGGCTTTGGTCTNGCATGCCCAGACCTTTTTTGGCCCCTCTGTGCTACAGATTATACACC
TTTGCGATGTATCCTCCATCCATGATCGCCACAGGCAGCATTGGGGCAGCCGTGCTAGGCCTAGGCGCCTGCTCTATGTCTGCGGATGAGCTCACAG
AGTTGCTGGCCGGGATCACAGGCACTGAAGTGGACTGCCTGCGAGCCTGCCAGGAACAGATCGAAGCTGCCCTCAGGGAGAGCCTCAGGGAAGCTGC
TCAGACAGCCCCCAGCCCAGTGCCCAAGCCCCCCCCGGGGGGCTCTAGCAGCCAGGTCCCAGTTGACCAGCACTCCCACAGATGTTCAGCCATTCAC
CTGTAG
HUMAN SEQUENCE—GENOMIC
CACACCACTGCACTCCAGCCTGGGCGACAGAGCGAGACTCTGTCTCAGAAAATAAAAAAAATAAAAAATAAGTGGGATCTATTATTATTACTACCTA
ATAGTATTATTACTATTATTGCTCTGGTGTGCTATCAGGGGTGTTGTGCCCACACTGTGACATACTACTCCTGACCCCCCTCCTTTCTTTTCTTTGG
ATGCCCTCTTCTTCCTGCTTCTCTCTTCTTACTCCCACTTTTCTTTTTTTTTTTTTTTTTTTTGTGTGTGTGTGTGTTGTGTTGTGTCGCTCAGGCT
GGAGTGCAGTGACATGATCTCAGCTCACTGCAAGCTCTGCCTCCCGGGTTCACGCCATTCTCCTGCCTCAGCCTCCCGAGTAGCTGGGACTACAGGC
GCCTGCTACCATGCCCGGCTAATTTTTTGTATATTTAGTACACACGGGGTTTCACCATATTTCTGGTTGGTCTCGATCTCTTGACTTCGGATCT
CCCACCTTGGCCTCCCAAAGTGCTGGGATTACAGGCGTGAGCCACCGCGCCTGGCCTACTCCCACTTAATCTCTAAACTCCTGCAATCCCATTTCTT
TCACCATGGCTCTTTCATGGAATTCAGGTGATCAGAACCACCAATCTCCACCAGTCCCAGGACAGCCCACCCTTCCTGTCACAAACCACCCTGCC
TACTTCTGATGGGTGTTGCAAATGCCAGGTAAAGTCTTCTGTCTTCATGACATCTTAGGATTCCGCATCATGGAGGATCATCATATCATGCACT
TTAGACTGGAAGCTTAAAAGGGTGGCTTCTCGGCCCAGCGCAGTGGCTCACGCCTGTTTCCCAGCACTTTTGAGGCCGAGGTGGGCTATCACGA
GGTCAGGAGATCAAGACCATCCTGGCTAACACGGTGAAACCCCAACTCTACTAAAAATACAAAATTAGCTGGGCGTGGTGGGGGCGCCTGTAGT
CCCAGCTACTCGGGAGGCTGTGGCAGGAGAATGGCGTGAACCTGGGAGGCGGAGCTTGCCGTGAGCTGAGATCGCGCCATGCACTCCAGCCTGGGT
GACAGAGAGAGACTCCATCTCAAAAAAAAAAAAAGAAAAAAAAAAGGGAGTCTTCTCATCTCTGACCCATGTCCAGCATGTCCTGTTTCCGTG
GCCTGCCTGATGTATGGAAGGCACATGATAAGGCACCTGATTGTTAATGACCACAGCTATCTTTATTGACCACCTACTATGAGCCTGGGACAGCTAA
AGTACTTTTCCCTTATTAATCCCCTCATTCACCATATGAAATAGGAGTTGTTACCCATCCTCCTTTTGCAATGAGAAGGCTGGGACGGAAAAGTTAA
GTAAACTGCCCAGGATCACACAATCACACAGCTGGCAAATAGCAGAGCTGGGCATCAACCTAAATCCAGAGCTCACTTACTTAATGACTAAGCCAAA
CTAATGCCAGTGCTATCTCCTGCCTAGATGAGGATGCTTGCCAGCCAAGAACCGTGATGCTCTCTTTTTTGTGCAGGGTATATTGCTGTAGCAGGTT
ACCCTTCCACACCTGAGGCCCTGTGTGCACCTCAGTCTTACCTGTGGAACAGGAATCTCAAGCAGAATGACAGAACAGGAAAGCCTCATTTATTCAG
AACCCAGACAGCCAGAAAAAGGAAGAAAAAGCAAATGCTTAAATATATAGGTTGTATTCTTTTTTTTTTTTTTTTTTTTCTTGCTCTGTTGTCCATC
TGGAGTGCAGTGGCGTGATCTTGGCTCACTGCAAGCTTCCCGCGGGTTCACGCTTTCTCCTACCTCACCTCCCGAGTAGCTGGGAACTATGGCACCC
GCCACCACACCTGGCTATTTTTTTTTTTTTTTGATATTTTTAGTAAAGACAGGGTTTCATCGTGTTAGCCAGGATGGTCTCAACCTCCTGACCTTGT
GATCCGCCCCGCCCTCAGCCTCCCAAAGTGCTGGGATTACAGGCTTGAGCCACCACACCCGGCCCTGTAGGTGTATTCTTATTTTTATTTAGAGACT
TGACGTTTCAGGCTAGTACCTGAGGTTTCTTTTTCCCAAGCAGCTTATAAGCTAGAAATTGCAAACTGCTTTTTGTCCCTCAGACCTGGTATATAGG
CATATTATGTGTGGCTTGCACGGTTTTGTTACTGTTTTTCAACTCAAGTAATATAGATATTTTTAGGAAATTTCTTTTTTTTGTTTTTGAGACGGAG
TCTCGCTCTGTCGCCGCCTCCCAAAGTGCTGGGATTACAGGTGTGAGCCACTGCGCCTGGCGGAAATTTCTTTTCTTTTGAGACACAGTTTCATTCT
GTCCCCCGGGCTGGAGTGCAGTGGCACGATCTCGGCTCACTGCAACCTCTGCCTCGTGGGTTAAAGCGATTTTTCTACCTCTGCCTCTTGAGTAGCT
GGGATTACAGGCGTGCGTCACCATGCCTGGCTAATTTTTTTGTATTTTCAGTAGAGATGGGGTTCTCTATGTTTCTGGCTGGTCTTTAAACGCCTGG
CCTCGCTGGTCTTGAACGACTGGCCTCAAGTGATCCACTCACCTCAGCCTCTTGTGCTGCGATTATGGCATGAGCCACCACACCACCCCAGCCTTTT
TCTTTTCTTTTTGTAGTATGGGGTCTTGCTGTGCTGCCTAGGCTGGTCTTGAACCCCTGGCCTTGTGATCTCCCATCTTAGCCTCTTCAAATGTGCA
GGGATTGCAGGCGTGAGCCATCATGCCCGATCTAAGAATATTATCTTGGGGAAACAGGGAATCTATGGCCACACTCATTACTATGGTGTAGCAAAAA
GTCTGAAGCTAAGCAGTACTTTTTTGAGCTTTGGATGGGACATGTGCTATCTACTGTGTCTGGCCCCAACAGCTCCCTTAACCCATGTGAGTTGCCA
TTTATCATGTGGTGTATGCTGGTGTTTTTCTTTTTTCTTTTTTTCTTTTTTTTTTTTTTGAGATGGAGTCTTTTCTGTAGTTTCCATCTGGAGTGCA
GTGGCACGATCTCAGCTCACTGCAACCTCCGCTTCCCGGTCCCGGTTCAAGCAATTCTCTTGCCTTAGCCTACTGAGTAGCTTGGGATTACAGGCAC
GCGCCACCATGCCCAGCTTTTTTTTTTTGTATTTTTTTACTAGAGACGGGGGTTTACCATGTTGGCCATCTGGTCTTGATCTCCTACCTAGTGATCT
GCCTGCCTCGGCCTCCCAAAGTGCTGGGATTACAGGCGTGAGCCACCGCGCCCAGCCTGGTGTTTTCTTATATCCAGTTTGTTTTTATTCATTAACT
CAAAAAAAGATTTATTGAGCCAGACGTGGTAGTGCACGCCTATAGTCCCCAGCTACTTGGGAAGCTGAGGTGGGAAGACCACTTGAGCCTGGGATAG
AGGCTGCAGTGAGTCGTTATCATGCCACTGCTTCCAGCCTGGGTGACAGAGTGAGACCCTGTCTCAAAAAAAAAACAAAAAAAACCAAAAAAACTTA
CTGACATCTACTATGAACTGGATACCATCCTGTTTGGTGGTGTGGTGTGGGAGTAGACAAGCTGTAAACAGAATATGAACTGCAAAGCTATATGCTA
TACTAGTGATCAGTGGTAAGCAGAAAAATAAAGCAGGGAAAGAGCATAAGAAGTGTCAGGGCTGGGGAGCTGGCATTAATTTATTAGGTAGGATGGC
CAGGAAAGGCTGTAAGAGGAATGCAACTTTTGGACTTCCTGAAGGAGTGAGGGAGTGGGCCATGTGGATATCCGGAGGCAGTCATTCCACCAGGAGA
TGGAACAGCACGTGCAAAGAGAACATGTCTGCCCGGTGTGTTCCATGAACCGTGTGGCCAGAGCAGAGTGAGGGAGAGTAGTGGGAAATGGGGTGGG
AGTATAGATGCTGCAGGGTCTTTGGCAGTCACTCCAAATGAAATGGGGAGCCTCTGAGAGTGATGGGTTGAGACTAGACTGGAGGGCCAAGGGTGGG
TCAGGGAGACCCCGGCAGGAGGCTACCACTGTAATCCAGGTAAGAGATGATGCAACTGGGACCGGGGGGTAGAAGTGGAATCGAGAAAAAGAAGGGA
TTGGAGATGTATTTTTGACGTAGAATCAACCAGAAGTGGGTCGCAGGTTGGATGTGGGGACCAGTAAGGTCAAGGAAGAACTGAGTTTTGGACAGAG
GAAGTAACAGAGGAGGAAAAAAAGAGTAGATTTTGGAAATATTTTGAAATTATTTTAGCTCCTCTGTGATCTGCTGATCAGAGTGTTAGTAATTGTG
TGGGATTTGAGAAAGAGGAGTCAGGAATGACACCAAGTGTTTGGCCTGAGCCATTGGAAGGATGGAGTTGCCATTTTCTGAGATAAGCCTGTAGGAA
GAGCGGGGTTTTTTGGGGGAAAGATTAGGGATATCAGTTTTGGAGAAGTTGAGTTTAACTGGCCTATCGGACATCCTTGTGTTGCCAAGAAGGCAGC
ATTTTTAGTAGAGATGGGGTTTCACCATGTTGGTTAGGCTGGTCTCGATCTCCTGACCTCGTGATCTGCCCGCCTCGGCCTCCTGTGCTGGCCCAAT
TACAGGTGTGAGCCACTGCACCCAGCCAATTTTTGTATTTTTAGTAAGATGGGGTTTGCCATGTTGGCCAGGCTGGTCTTGTCTCCTGACAACTTCA
GGTGATCCGCCCCACCTCGGCCTCCCAAAGTGCTGGGATTACAGGTGTGAGCCACCGCCCCAGCCGAAGAGTGCTCAATTTTTACTGATAGGAATGG
ACAAGCAAAGAGTTTTAAAGATTAATGTGAAATATTGTACAAATATTACAGATGCTCATAATAGTGACCTGGATACTGTAAAGCACCAGGATAGCTC
CTTGATTCTCAAAGAAAAAAGCTAAATTATATGTAGTTGGGTTTACTGCTTTTTTTTTTTTTTTTTTTTTTTTTTTTTTGAGACAGGTCTCTGTTGC
CCAGGTGGAGTGCAGTAGTGCGATCTGGGCTCACTGCAATCTCCATCTCCCAGGCTCATTTCTCCTGCCTTGCCTCCTGAGTAGCTTGGGAC
TACAGGCGTGCGCCACTGCGTTCGGCTAATTTTTTTATATTTTTTGGTAGAGATGGGGGGTTTTTACTGTGTTGGCCAGGCGGTCAAACTCCTGGGC
CTCAAGTGATCTGCCTGCCCCCTGCCTCCAACGTGCTAGGATTACAGGTGTGAGCCACCGTGCCCCCGGCCAACTTTGTCTTTTAAGTCCCCAATCA
CAACTAACTTTTTTAAAAAAGGGTTCACCACGTTGCCAGCCACTCTTCTATGTATTTAAACAAATCAACCATTTCCTCCTCACAACCACTCTGTGGG
GGGGTACTACTGTTATCACCATTTTAGAAAAAGGAAACTGAGGCACAGAGAGAGGTGGTTTTAACTTAGCCAATTTCACAGTGAGGAGTGGAGTGGC
AGAGGGGTGTGGCTCCAGGCCCTGGAGGTCCTCTCCATACGCTAGACCATGTGTTTACCGTTAGTGCCATTCTTCATATTTGCTAGAGATCA
ATTCTAGGAGTAGGGCTCTTGCTGCTCAAACTTTTTGCTTTTCTTAGAGGGGTTATGGATGCATGTTCATAACAGAAAGGGTACTCCTTGAGAAGGG
ATTATAGGGCTATTATATTATCCCCACTGGATAAAAGAGGAAGCAGAGAGGTTCCAGTAATTCTCTCAAAGTCATTGCTGTGGAGCTTTTTTTTTGT
CTGAAAGCAGTCTCTGACTCCAGAACCCTGTTTAGCACCCCTGTTCTCAGCCCCATGGCTCGCTTGGCTCTCCCCAGCATCTCAATGGGTCTGAAGG
TAACTAAGCCATGGCATGTGGACTGTGGGGGTGATGGGCCTCACCCATCACCTTGTTCAGCCTCTCACTTCTCAGATGCGGACTTTTTTTTTGGAGA
TTACTAAGCCATGGCATGTGGACTGTGGGGTGATGGGCCTCACCCATCACCTTGTTCAGCCTCTCACTTCTCAGATGCGGACTTTTTTTTTTTCAGA
CAGAGTTTCGCTCTTGTTGCCCTGGCTAGAGTGCAATGGCACGATCTCGGCTCACTGCAGCCTCTGGCTCCAGGGTTTGTGATTCTCCTGCTTTCAG
CCTCCGGAGTAGCTGGGATTACAGGCATCCACCACCACACCTGGCTAATTTTTTGTATTTTTAGTAGAGACTGGTTTTTACCATGTTGGCCAGGCTG
GTCTCGAACTCCTGACTTCAGGTGATCCACCCGTCTGGCATCCCAAGTGCTGGATTACAGGCGTGAGCCACCGTGCCTGGCCTGATGTGGGGGGGTA
TTTTGATGATGAAGAGGAAGAGAAGAAAATGTATCTTTCCTCCTTGGGGTTTCTTATTTGGCCTGTGACATTCCATTCCTCCCCTCCTCTATTTTTT
GCCATTTTATTGACTTTTTCTAACTGGATGTACCTGGGTAGAAACTCCTCATACTATTGGCCCTGGTCATTGCTTTGTCTTTAGTTTTCCTTTTAGA
AAAAGTCAAAAACAAAACAAGAAAACAAGAGGAGCCTCTATTTCTTTGAGGTGGAGGTAACTGGTGTACTTGTGCTGGTCCTGCATTGAGCTCACCT
GCCTCCCCAGTGGCCAGTCCTTGGCCCCTGAAGTCAGGGATGATCTAGCTCTAGGCATGGCTATTCCATCCAGGGGGGTGAGTAGTGTATCTCTCCT
AAAGTCCTGCTCACCAGGAGCTCCCGTCCCCCATACTACAGGTTCACATCCAGCTTTCAGGACTAGTCAGTCTATGTGGCCCTCCTCAATTAATAAA
TCAGCACTATTTGCCAGGTGCGGTGTTTGTGCCTGTAATCCCAGCACTTTAGGAAGCTGAGGCAGGCAGATCACTGAGGTCAGGAGTTAGTTCGAGA
CCAGCCTGGCCAACATGGTGAAATCCCCGTATCTACTGAAAATACAAAAATTAGCCGGGCATGGTGGTATGCACCCCGTAACCAGCTACTCAGGAAG
CTGAGGCAGGAGAATCACTTGAAACCAGGAGGCAGAGGTTGCAGTAAGCTGCACTCCAGCCTGGTGACAAGAGCAAAAACTTTGTGTCAAAAAACAA
AGAAAACCAAAAAACAAAGAAAAACACAAAAAACCCTTCTATTTGTTAAAAAAAAAAAAATCCACCGTGAACCAAAAATTAGTAAAAACAATGAACT
AAAATTTTGTTTTTGCAAAATGTATGATAACAAATGTTAAGGAAGGTCATGTGCCGTTATGGTTCACTGCAGCCTTGTCTCCTGGGCTCAAAAGCGA
TCCTCCTGCTTCGGTCTCCCTAGTAGCTGGGACTACAGGCTTGTGCCACCGCACCCAGCTTATTTTTTTTTTTTATTTTTTGTAGAGATAGGAGTCT
TGCTTTGTTGTCCAGGCTGGTCTTCAACTCCTAGCTTCCAGTGATCCTCCTGCCTCAGCCTCCCATGTGCTGGGCTTTGGGACATTTTTTTATACAT
AGTGCCATGTTACTATAAATGAGAAGTTTTAAAATACTGATTTTAAAAATTAATTTATGTCAAGAATTTTTTATAACCAAAGTTAAAAACCAAAACA
AAAAATATGAAAGGGTTAATATCTTTGAGAGGTGATGAGAACTTATAAGTCAATAAGAGAAAACAAACATCCCTATAAAATGAATAAGCTAAGGACA
TGAATGGGTAATGTACATAAGAAATGTAAATGTCTAGTAATATGCCAAAATAGATTTATTATTACTAATAAGCCACTTTCACTCTCTAGTTTGGAGA
GTTGTTTTGAAAAATAGATATGTAATGATGGTGGAAAGATTGGTTTAACTATTCAGCAGGAAAATTTGGCAATTAGAAGTGTATCAAAAAGCCTTAG
ATTGTTTCATAACCTTAGATTGGGAAATTCCACTTCTAGAAATTAATTCACTTCTAGAAATAATCATGAGTGTGCACAAAGATATTACCACAATTTT
ATTTTACAGTATTATGTCTAATAGAGAAGAACTAGAAATAATTTAAATTTCCACCAATACAGGTTTGCCAATTACATTTTGTACATTTCCAAATTTG
GTATATTATGTCCCTATTACAAATTATGTCCTAGAATATTTAATAGCATGGAAAAGTGTTAACAGTATTTTTTTAATGAAAAAAGCTTACAAAACAG
TTTGTGATGATTCCATTTAAAATGTGTGTTTATTCATAGAACAAAGATTAGAAAAATAAACATTGATATATTAAAGGGTTATTTCATGGCAAATTGC
AAATGATTATTTCCTTTTTTTGTGGCTTATTTGTATTTTTGAAGTTTTCTACAATGTAAAAGAATATTTTATGATATGAAAACTACAATACAATTTA
TAATATAGAAAGAATAATTCGGCCGGGAACGGTGGCTCACGCCTGTAATCCCAGCACTTTTGGAGGCCGAGACCGGCGGATCACGAGGTCAGGGGGT
TCAAGACTAGCCTGGCCAACATAGTGAAACCCCATCTCTACGAAAAATACAAAAATTAGTCAGGCATGGTGGTGCGTGCCTGTAGTCCCAGCTACTC
GGGAATTGCTTGAACCCGGGAGGTGGAGGTTGCAGTGAGCCCAGATCGCACCACTGCACTCCAGCTTGAGCAACAGAGTGAGACTTCGTCTCTACTC
ATAAAAAAAAAAAAAAGAATAATTAACAGAAAATGGTTAGACACTTCCTTAGTGTCTCCTAAGTCAGGAGGACCCCAGTTGGGCAGGGATCCTCAAA
TGGCCTCCTCCCATTTGGAGCATTATTGGAGGTCTTTTTCGGCCTCTTCGTCAAGTGGAATCTAGCTTCCGGTAAAACTACAAGTAACTATAAAGTT
TGGGAGGTGGAAGAAATGCAACCGGTAGATCTCACAGAGTCTGTGCAAGAAACTGATTCAATGAGAATCTAGTTTCTCCGTCCACAGTTTCTCCAAA
CAGAAACTAAGGCCGACTTTAGGGGCTTGTCCAAACCTAGGCAAGCAACTTAACAAGGTGAGGCCATGACTCCATGGCCTTTCCGTTCTGTTATATG
CTGACTTAGACTAAAGCTCTCATACTTTAAAGTGCACAGAAATCTAGTTAAAATGCAGATTCTGATTCAGGTTAGGGGTGGGCCTGAGAGTCTGCAT
TTCTAACCAGCTCCCAGGCGATGACCACGCACGGGACAGGTCTGGGATCACAGTTTAACTAGCAATGGTGTAGAACACAGAATCTGCAGCAAGAAGG
CCAGCTTCCCATTCCTAGCTCTGCCACGGACCAACTGAATGACAGTTGCCTCGGTTTCCGAGTTTTCGTGAAGATGTAGTGAGTCATTACATCGTGA
GGCTTTCGAGCAGCGTTCACTAAGAACTAGCTCTGACATTATTTATCGCATTCCTTAGAGCAAGCAGCCGGTGAAGTAGGGTTTGACGAATGAATAA
GTGAATGAATGACCTTTGGAGAAAAATTGTTTCCTGGGTGACTAGAGTCCGAGAAGCAAAATGGGAGGGCCCGTGGGTGGGTAGGAGGCCCACCTCC
TAGAAAGTTCTCTGCACCCGGTGGTCCAGAGGGCCTGGAGTGCCCGGAAGCCGGCCGGCTTGCGCTCAGCGGCCCAATGGGGCCGCGGGAGGGAGGG
GAGAGCGCTCAGCCAACCCTTTCCGTCCGGGGCGCCGCAGCCCCGCCCCTCGGAGCGTTGCGACGTCCGAGCATTCCACGGTTGCTACATCGTCGCG
AGGGGCGGGGCGCCTGTCAGGGAAGCGGCGCGCGCGCGCGGGCGGCGGGCGGGCTGGGGATCCGCCGCGCAGTGCCAGCGCCAGCGCCAGACCCGCG
CCCCGCGCTCTCCGGCCCGTCGCCTGCCTTGGGACTCGCGAGCCCGCACTCCCGCCCTGCCTGTTCGCTGCCCGAGTATGGAGCTGCTGTGTTGCGA
AGGCACCCGGCACGCGCCCCGGGCCGGGCCGGACCCGCGGCTGCTGGGGGACCAGCGTGTCCTGCAGAGCCTGCTCCGCCTGGAGGAGCGCTACGTA
CCCCGCGCCTCCTACTTCCAGTGCGTGCAGCGGGAGATCAAGCCGCACATGCGGAAGATGCTGGCTTACTGGATGCTGGAGGTACCGCCCGGACGCG
TCCCCCTCCCCCACCGCTCCCGGGTCTGGACACACCGGGGCCGCCCTGTCGGGACATCCCGGTCCCCACAGCAATCTGCAGGATGCTCCGAGGCTAA
GACTCCCGGGGCCTGGCCTTTCACTAGGGCACCCACGCCAGCTGCCTCCCTTGCTTTCCCGAGCCCTAATCGGCTCCTCCCGACAGTGACTGCCAGT
CACTTGGCACCCTAACTGTGCCTCTCTTCGGGCCGAGCAAACGGGACCAAGGACACTCTCTCCCCTTCGGCTTCTCACAGCGTCTCCCGCTTCCCTC
CGCCTCCCGGCCTTCTCCGGAGAGCGCGCCTGGAAAACCCTGGGCTCGGCCGCCCCCGCCCGGGAGCCGGTTCCTGTGTCTTTCGCGGGGTTCTGGC
GGGGAAGGCGATGGGGGTGGCGCGCGCCCTGTTCCGGCCGACAGGGAAATGAAAAGTGCAGGAGGCAGCGGCAGGAGCCTCCTCCCTTCCCCTCGGG
TGTGTGCAGTGGTGGCGGTGATGGGGGTGAGGTCCCAGCTCTCTGGCTACCATGGGTGACCCTTGCGTCCCACTTCCCCTCGCTGTGGGGGAGGGGA
GTCTCCGTTTGGAATCCTCCGTGAGCAAGGGCTCCTTGGCTTTACCACTCCCCCGTCAAGGGGAGAGGGGAGGAGGCCGCGCAGAGGAAGTGGGGGG
CGTCACCTTGGGGAGGGCGCATCCTCCTCCTTTATTGGTCCTTGTCTCTCTAAGTAGCTTACCCTCTCTGCCGTTTTTGCTCCCTATCCCACCTCAC
TTCAGCGCTCCCACCCCCCCGCTGCTGTGTTCCCCACGCTCCAGACCCAGCAGTGACTGTGCGCCCTCCCCCAGGTATGTGAGGAGCAGCGCTGTGA
GGAGGAAGTCTTCCCCCTGGCCATGAACTACCTGGATCGCTACCTGTCTTGCGTCCCCACCCGAAAGGCGCAGTTGCAGCTCCTGGGTGCGGTCTGC
ATGCTGCTGGCCTCCAAGCTGCGCGAGACCACGCCCCTGACCATCGAAAAACTGTGCATCTACACCGACCACGCTGTCTCTCCCCGCCAGTTGCGGG
TGCGTGTGTAACTCCCCCCACCCCCGACACGTATTGTCTCCCCACTTTCCAGGAAAAGGAAAGCTTGAGATATCAGAGGCTTTCTGCCCCCTGGAGA
GGAAGCAAGGGGATCAGGACCTTTGGGAAAGGAATTGGGGTGGAACAAACGATGGGGCATTGTGGTGTGATGGTTAGTATCACTCCCGCCCAGGCTT
GAGCTGCAGGCAGGCATTAAACCCCCTCCTCCTGACCTCCAGGGGATGCCACTCCTTCCTGGTCTTTCCCCCAGATAGCAACAAGTCAAATTGCATG
TCTACACTCACTAGTACTTTGCCTCTGGGTTTACGCAGCCACCATGAGTTATTTATTGGCAGCAGAAGGGCATGCCTGCACACCCCCACTGGAGTAG
GGGAGTGGAATGTGCTTCATGTGGGGGAGGGGGTTTTGCCTTGGTTGAGTCTTTATCCTCATGTGGACTTTGTGATAGAAAAGCCTCCCAGAGCCAA
TTAAAGCCAGGCCCTGATTGCTTTAAGGAAGGGCTGATGTATTTCATCCCACCTTCTTTGCTTCCTCTTATATCCCGGAAGAAACAGCTTCTTCCTA
ATCCTTCCTCGTCCTCATTCTGAGGAACTGGAGCAGCTGCTTTGGGCCTGGTGGAAACAGCTCCCTCTTCCCCCCTTAGCCCAAGGCCCAAGCTGAG
GGACAGACAGGGAGGTGAGCTGCATCTGCTTCCGATGTGATAATGGTGCCCCCTCCCCTGGCCCGCCCTCTCTGACCAAGGTCTTTCATAACCCTGT
GGGGAGCTGCTGTCACTCCTGTGGTTTGGGAAGTGGCTCCCTGGGCCTCTTGCTGGAAACTTGGGCTGGGCTCAGGCCGGGCAGGAAAGTCACCGGT
TCTGTCCCGCCTCAGAAGGAGCCTGGGGAGGAGGCGGGGGAGCTGCTGGATCTGGAAGAGGTTATTTAGGACATGCAGATTGTCTCCTAGGGAGGCT
GGGGGAGAGGGTGGGTGAGCTGGCACCTTCCTCCACCTTGCTCTGTGGCGGGCCAAGTCTTCTGCTGACCCTGAGAGGAGTCTCTGGGCCCCATCTC
CCAGGGAGGAGGGCAGCAAGCAGGCTGCTGTGATTGGCTGGCAGGGAAGCCAGGGTACTGGGGCCCCCGACTGCTGTGAGTCGAGGAACAGGATTCC
TCTTCCTGCCAAGTAGCTCAGAGATGGGAGTAGGAGGCCAGAGTTGGGAATCCAGGAGGCGCCACAGCTCTTTGTGATGAATCTGGTAGTTTTTGGC
AAGTCTGAAGGTGGAGCTAGTTTCCAGGGCCAGAGAGCAAGATGTTTTTGGAGGGGTTGGGGCTGGAAGGTAGAAGGGGAACTTCCTTTTTGTGAAA
AGATGGAGGTGGTTTGTGTGCCCACGTATGAGGGTTTTTCAGTACAGGCAAAAAGTACTCTTCCCTCTGCCTGAGCTGGGGGTGGAGTGCCCAAAAG
ACTTCCTAGAAGGTCAAGGTTGGAAGGGACCTCTGAGAACTCCAGTTGGTCCAAATCCGTGTGCAGCAGAGGAGGCTGAGGCCCAAGGAAGGAGGGC
AAGGTCCTGCCTCAGCATCGCACTGAGCAGATGAGTGTAGAACCTGGGTCTTCTAGCTAGACCAGGTTGCCTTAAAAGTCAGGAATGTGATGGCCGC
TGCCACCTACTGAAGCTAAGCACTCCCAGGGGTGTGTTCTCCACCAGTTAACACTGGGATTCTCTGGTTGCCTGGATATGGGGAAAGGTGGCCACTT
TCCCTCAAACACAACCCCTTGCCTCCCCATAGGGAGAGTCCCACTCCCAGTTGCCTTCCAGATGCTTCCTTAGAAGCTGAGTCCCTTCTGTTTACTG
CTGAGTTAGGAGTTGGCTCCTGGGACATGCACTAGGGGTAGCGTTGTAATTTTTCTGTCTCACTTCTGGCTCCAGGCCAGGCTATTTTCTCTGGGGC
TCTGTGAGGGTAGGGGTTGATTTGAGAGGCTGACACCTAAGCTGGGGCCAAGAGAGAGACAGCTGTGGCTGGGGCCTGGAAGAGGTTGGGTGGGGTT
GCCATTTCTGCCTCAATAAGGAGTCAAACCCACAAGTTCCTTCCTGAGATAAAACAATGAAGAAATCCTCATGTGTGCAACTCAGAAATGGGGCCAG
GTCTGGGAACCCATGACGAGTGGACTGGGGTGGAGCTCCCAGCCTTCTCCCGAGACCTAGTACTGGCAGGTAGGCTTGGGTAGGTGAGGATAGGGCT
TGATCCCTCTGGAATCATTTGGAGCCATCCTGTCTGGCTTGGCCTCTACCTATCCTCCTGAAAGTCAAGTGGCATGGGGCACTACGGGTTCCAGCTA
AAGGGGGTGGGGTGGGGTGCTATACCTCAGGACCCATCCCTGATATCCTACCAAGGGGATGTGGCTTGGGTTGGGCAATCTCCCCTTTGAGGAACCT
GAGAAAAGGCTCTGTGGCTGTCATTGGAGGAGTCATACTCCCTTCTGTTGATTCAAGAAGTGTGGACTTGGCTGGGATGCAGAGTTGCAGGGGAAGC
TAGTGGTGATCTCCCCTTTCACAAATCCTTGGGGTTGAGAGAGTCTTTCTGCTTCATCCCAGAAGAGAGCCCAGTTCATGGTGATGCTGTGGTATCT
GCTGCTTCCGTCTTTCAGCCCTGGCTGGAGAAATCCTCAGAGCCCTGAATGAAGTCAGGAGAAGGCCTGGATTATTGGCCCCTTAAACATAACCAGA
CAGGATTAAAAGGGATAATCCATGTAAAGCATGTTGTCCTGGCACCTAGTAAATGATCAGTAAGTGCTCAAAGCACATGCCTCCTCCTCTTCCCTGC
CTTGAACTCCATGGTGCCCGGCCTTAAAATCAGAAACAGAACTGGGGTCTTGGGCTTTAAAAGTTTGATGGGCTTCCCTGCCTGAGACTTTGAGACT
TCTGCTTTGCTCCCTCCTCTTCTCTTCTCCAGCCACTGACCCCACCCTTTCCCCGCTTTTTCAGGACTGGGAGGTGCTGGTCCTAGGGAAGCTCAAG
TGGGACCTGGCTGCTGTGATTGCACATGATTTCCTGGCCTTCATTCTGCACCGGCTCTCTCTGCCCCGTGACCGACAGGCCTTGGTCAAAAAGCATG
CCCAGACCTTTTTGGCCCTCTGTGCTACAGGTAAGAGCCCTATGCACATTVTTGCCAAATTGGAAAAATCAGAAGACTTATAAGGGTGAGAGGTAAC
TGAAGATTCTGGTTTATACTGTCAAAATTCCTTTGTGAAAATTATACCCCCTCTGAGCCCCCAGTCTCAATGCAGAAAAGCAGGTTATAATCAGCTA
TATGGGATCTCAGACCCCTCCAGTTTGTAGTGGGGACACTGAGGGTATAGAGAGAAGTGACTTGCCCAAGGTCTCACAGTGAACAGGTTGGGAACAG
ACCCCAGTCTCTTTGGCCCTAGTTGTGTTCCCATACTAGGGAGTGTCTTGTCTATTCCCGGGACCTATGGAAACTGGCAGTTGGTTCTGATCTCTAG
GAAAGATACTCTTTCCTGTCTTCCTCCCTTGCTGATATCTTGCCCCACCCACTGCTGGAGTTTTCCAGCATGTCCTCTGCTCTACTCATGCTGCCTG
GCACTTTCCAAGGAGTCGGAAGTCCATGCTGGCCGTTATCCTTCAGGTTTCCTTCTCTCATGGTTCCCTTTCCTCCTCTCCAAGATTATACCTTTGC
CATGTACCCGCCATCCATGATCGCCACGGGCAGCATTGGGGCTGCAGTGCAAGGCCTGGGTGCCTGCTCCATGTCCGGGGATGAGCTCACAGAGCTG
CTGGCAGGGATCACTGGCACTGAAGTGGTGAGTGCTGGGTAGCTGGGCAGCAGCCTCTCCATTCATAAATTAGTGCTTTGCCCTGAGCCTCCAGTAG
AGGGAAACCTCCAACCCCAACCTGGTCCCAAGTCCTGGTTTCACAACCTTGGGGCTTTGTGGCTTTTTATCTCCTGCCTTGGTCTGGGGAAGAGCAA
GTATCTTGGGTGGGGTGAAGCGCACTTGCCCTAGTGGTTCTTTTCTTGCCAAGTGGTGTGCTGGAGTGGGGGTGGGGGGTTTGGTGTCAGATGCAGG
GCTGCTCTCACACTCCCTTGCCACATCCTCTTGCTAGTTTCTGAGAACTAAAGAGCGATTCCTGGGGCCGGGCTGTGGCCTAACCTCCCCAGACTTC
CCCATGTGTTGGGAGCTGTCCTTCCCCTGCACCTGCTGCCAGATGCTATGGGGGGAGTGTTTGCTCCTCACTCTTCTCCCATGTTCCCAGGACTGCC
TGCGGGCCTGTCAGGAGCAGATCGAAGCTGCACTCAGGGAGAGCCTCAGGGAAGCCGCTCAGACCAGCTCCAGCCCAGCGCCCAAAGCCCCCCGGGG
CTCCAGCAGCCAAGGGCCCAGCCAGACCAGCACTCCTACAGATGTCACAGCCATACACCTGTAGCCCTGGAGAGGCCCTCTGGAGTGGCCACTAAGC
AGAGGAGGGGCCGCTGCCACCCACCTCCCTGCCTCCAGGAACCACACCACATCTAAGCCTGAAGGGGCGTCTGTTCCCCCTTCACAAAGCCCAAGGG
ATCTGGTCCTACCCATCCCCGCAGTGTGCACTAAGGGGCCCGGCCAGCCATGTCTGCATTTCGGTGGCTAGTCAAGCTCCTCCTCCCTGCATCTGAC
CAGCAGCGCCTTTCCCAACTCTAGCTGGGGGTGGGCCAGGCTGATGGGACAGAATTGGATACATACACCAGCATTCCTTTTGAACGCCCCCCCCCCA
CCCCTGGGGGCTCTCATGTTTTCAACTGCCAAAATGCTCTAGTGCCTTCTAAAGGTGTTGTCCCTTCTAGGGTTATTGCATTTGGATTGGGGTCCCT
CTAAAATTTAATGCATGATAGACACATATGAGGGGGAATAGTCTAGATGGCTCCTCTCAGTACTTTGGAGGCCCCTATGTAGTCCGTGCTGACAGCT
GCTCCTAGAGGGAGGGGCCTAGGCCTCAGCCAGAGAAGCTATAAATTCCTCTTTGCTTTGCTTTCTGCTCAGCTTCTCCTGTGTGATTGACAGCTTT
GCTGCTGAAGGCTCATTTTAATTTATTAATTGCTTTGAGCACAACTTTAAGAGGACGTAATGGGGTCCTGGCCATCCCACAAGTGGTGGTAACCCTG
GTGGTTGCTGTTTTCCTCCCTTCTGCTACTGGCAAAAGGATCTTTGTGGCCAAGGAGCTGCTATAGCCTGGGGTGGGGTCATGCCCTCCTCTCCCAT
TGTCCCTCTGCCCCATCCTCCAGCAGGGAAAATGCAGCAGGGATGCCCTGGAGGTGGCTGAGCCCCTGTCTAGAGAGGGAGGCAAGCCCTGTTGACA
CAGGTCTFTCCTAAGGCTGCAAGGTTTAGGCTGGTGGCCCAGGACCATCATCCTACTGTAATAAAGATGATTGTGAAATAAAACTGGCTTTGGCTTC
TTGGATTGGTGTGTGGGTAAGTCTATTTCTGCACTGTATGAGGTAAGGAGGTGGTGTCAATGGTCTTGCAGGGATCATTTGGAGCCTTTTCTTTGTT
AGGGCTGGATATTTAGGCTTTGTGGGGTACATGGTCTCTGACCCAATATAGTCAGTTCTGCTTTATAATGTGACCTATGCGTTCCTTAAAATTCACT
ATGCCATGCAAAAATTGCACAAATAAAGACCACAGGTCTCATGGGGAAATGGGGCTTGAAGCACAACACTCAAATGAGTGACACATTAAAAAAAAAG
GATAGCTTGGTTTTTACATATGTCAATGGTTGAGATATTATATCACAATAATATGGCCTTTTTATCTTTGACATGCACTTTTGCTTTTTTGGAAGTG
GACGTCGGAAGGGTTGCTCACAGGTGGGGGAGATGAGTGGAGGTAGCTGGTACATGTTTAGGTGTGAGTTTTTGTGTACTACTTAGGCTTGGTTCAG
CTAGATACAGTTTCCCATATTCACCTACTATTTCTCGAGGATGAAATCACACACAAGCAAACATGAAGTCTGCGTTATGATCAAACTGTTCCCAAAT
ATATTAAATGGGTTGGAACAAATGCGCAATTTCGAAAGCAAATAACAAGGGTTATAGCAGGATTATACTTAAACTCTGCTGTTGTAGCACAAAACCA
GCCACAGAAAACATGTAAATGGGGTGAGCATGGCTGTTCCAACAAAACTATTTATGGATGCTGAAACTGGAATTTCATGTAATTCCCTACCTCACAA
AATACTGTTTTTAATTTTTTCCCCATTTAAAAAATGGAAAATCATTCTTAGCTCATGGGCCACACAAAATGGGCACTTGGGCTGGGTGCAGTGGCTC
ATGCCTGTAATCCCAGCACTTTGGGAGGCCGAGGCGGGTGGATCACCTGAGGTCAGGAGTTTGAGACCAGCCTGGCCTCCTTTGGAAAACCCATA
CCACCCCGTCTCTACTAAAAATACAAAAATTAGCCAGGCGTGGTGGCACGCACCTGTAGTCCCAGCTACTCAGGAGGCTGAGGTGGAGAATCTAT
GAACCATGGAGGCGGAGGTTGCAGCGAGCCGAGATCATGCCACTGTACTCCAGCCTGTCGACAGAGCGAGACTCCGTCTCTTCAAAAAATCAACA
AAAAAAGGGGCACCTGGCCCTAGTTTACTAGTCACTAGTCTAAAGCTTTCATTGGCTGAAAAGCCAGAAAGGGAGCCTGTTGATCTAGCTCTGAT
CTAACCATTCTTGGGCCCAGCCTTATTCTCTGGCCTTTTTTTTTTTTTTTGAGACAGTCTGCTCCGTTCCCAGGCTGGAGTGTAGTGCCCCCCAA
TCCTGGATCACCGCAACCTCCACCTCCCAGGTTGAAGCGATTCTCCTGCCTCAGCCTCCCAAGTAGCTGGATTACAAGCACACGCCACCACACCA
GCTAATTTTGTATTTTAGTAGAGAAGGGGTTTCACCATGTTGGCCAGGCTGGTCTCGAACTCCTGAACTCAGGTAATCCACCTACCTCAGCCTCC
CAAAGTGCTGGGATTACAGGCGTGAGCCACCAAGCCCAGCCTCTCTTCCTTTCTTGACTCTTATCTCTGCTTTCCCTTAGTTTTCTCTTTTGTAG
TCCTTTCATCCACTGCTGAGACACGACCATCTTTGGACAACCTGCTCCTGGCTGCTGTAGCCTGTGATGGGGGAAGGCAGAGGATGTGTCTTGGG
GGCTAACAAGGTGAGTGCAGCCTGGAGAAACAAGCTTGGAGATCAAGGGAGGTGGGTCCTATATAAACAGGACATGTATGTCCTCCGTCCTCCAT
ACTAACAGGAGTAATGCCTACCTCCATTGACTACACCAAACACAGAGGTTCAATAAATATCTGTCTTTGCCTTTATCTCAGTGACAGACACAATA
GCCATAGAAAAAGTACTCACACCAAGCCCCAGCTGGGGACTGGGAATGCTAGCATATGGTATCTCTGTTGGCTACAGTCTTTCATGGGGATAAGG
GCCTAGATGGAAGAGGGAACCAGCCAGACCCTCAGTCCTTCCTGTCCTGGAAGGGAGCTCAGATGTCCAGTGATCTGTCACTGCCCCCTGATCTG
GGTCTTCAGCCATTAAAGCTCAGTGTCATCTTCAGTCACCAACGGGGGTCTTGGTGTTCCTTCCWCCCCTTGGCCAGGACAGCTGACTGTTTTCC
TCACTCCACGGTGATGGGACATCTTCCACATCGCGGGGGACTGCACTCTGAAGCTCACGCCTGATTTCTGCGATAGCTGTGGATTTCTGTTTTCC
CGGAGCAGTTCTAAGAGGGCCTCTTTCTGGTCTGTGGCCAAGTCGGCCTTGTAGCGCTGGACCNGTCAGGAGGCACTGGTGCCATGGGCATGGCA
GTTCACGCTTCTCTGTCCGGACCCCAGGAAGTGGAAGACTAGGGCATCCAGCACCCGGTTTTGTGCATACTTCTTATCCAGCAGCAGTCGCAGGA
GAAGATGCTGTTGGCACCGCTGTATTCCATCTCAGCAATTTTCAGCATGGCCGCACTAGTGGGAAGGGAGGTGGGGTAGAGTAAGCAGGGAAACA
TTGGAAGAAGGATCCTACTTCTCCCTCTATGCTTTCCCCTTTTTTTCTTCCCCTCTGCTGGCCATACATATGGTATTCCCCAGGGACCTTTCTTC
CTACACATCATATTATACTCCCTTGACCTCAGTCACCATCTCTACAGGGATCCACCCTTCTCTATCTCTAGCATAGACTGCTTGTTTTCAGTAAC
CAACTAGAGTATGGTCCAACATCTCAACGTCTCCACCCTAACTACTTCTTCCTGTGTTTCCTACTCATTTGTTTATCTATTTACTGAGAACTTAC
TTTATGCCAGGCACTCGGGCCCTGTTGTTGCACAACTTACATTGAGGGGATAATGATACACCTACTCATTGGCTTAAACAAGCACAACCCAAGGC
TTTCCTTAGCTCCTTCTCCACTCTGCCCATTCCCAAGTCCTGGCAATTCCACTATCTGGATATCCACTGACCAGCCCCCTTCCACCCCCTGTTAC
TACCTGGAGTGCACACAGGGATGGAGCACTTGGTGATGATGCTACCCACAATGATGGCTTCCCGGAGGGTACTGTGCCAGACTCGCACAGTGGAA
TCAGGATCCCTAGGGAGAGTCAAAGGGCAGCCACTGTGAGCCCAGGATGAGAAAAGGAGTTACAGCAGGAAGCATTCCACCCAGCCCAGGGACAG
CTAGCATTTCCCACACCAGGCCTGCTGTGTTCCCCATAGAGACCAGCCCTTCCCTTCTTCCGTGCCTCTGGGATCCTCCTTTGAACCACCAGGCT
CCAGGTTTGAAAAGGGCCTTCTTGAGAGCCATGTAGAGATGGAAGTTGAGTCGTTTGTATTCAGCTCGTCATCTCGTACTCGAGGGAGCATACT
GGTTGTAGAAGCGCTGGGCCATGTGCGTTCCTTCAGGTTAGAGGCAAATCCTAGTGGGAGAGTTAAGCGGGAAGTTCCAACTCACAGACAGTCTG
GACCCGGCCCATTAAATATCACCATGGATAGTGCATACAAGAGGTTTAAAGTAAAATATTGACCTTTCTTCAGTGTGGGATGTGTGGGAGGCTGT
GTGCATGCAACACCCATCAGCTTAGAGAGAGAATAGGAGGGCAATGAGATATACCCAAATGAGGAGCACCGTTTGTCTGTTGAGAGCCAGAGCTC
GTGCAAACTGATGGCTACTCAGCATCAGTTCGATTCTGCTAGCCCTGAGCATGCCTGGGCAGTACAGACTTCTGAGGTCCTGCTACAGGGTTGTA
TCCCAGTGGCTATAAAAGCCTCTCCCCTAGAAATTCATCACAGAGGGTTGTATCCTGTAGCTATTTAAGCTCTCTCCCCTAGTTTCATCACGGAG
GGTTCCTAACTGGCTAACTGGGTCTTGGCCCCAAAAATGACACAGGAAAAGCTAGAAAAATGATGCTATAAAGTAGCACCAGAGGGCCCTGGGGC
AAAGCTCAATGCCCTGGGGCCAGAGGCAAGGAAAGCCTGTCCGTCCCAAACTCAGCCTGCACCTATTCCCCTGGCCCCCCGTCCCCCCAGCTACT
CTACCTGGTGGCCTGGTACATGGCAGCTGCAGTCCAGGCCTCCGGCTCTGTGACGTAGAGGATTTGCTCCCAGTTGGAGAGTGTGGATGATCTTA
AATGCCTTGGGCAGTTTTCCACTGCGGTACTTAGATAATACCTAGGGATGAGGGAGGTAAAACAATCTCGTCATCCACATATGGCAGCTTGCTGT
GGGCTAGGAACTTGTCTGGTGCTTTTAATATATTAACTCATTGCATCCCCACAATAACCTATTGGAGGTGCCATTATTATGCTCATTTTACAGAT
GACCAGTGAGAGTTCCAGTAACCGACCCAAGGATGCCAAGTCATTTACAGGCAGAGCAGGAATCTGTCCTCACACTCTGCCTTTAGGTCTGTTTT
TCTACTCCCATGACTTCTTTAGTATGCGATGCAGGCAAAAGGCAAAAAGCCTGTGGCAGGAGCCCACAGCCCCCAAAGGGGTCTTCCACTTGGCT
CCCCTCTCAGCTCTTACCTCCCGGACCCCCCTGTACACTTCTAGGACCCGGGGGTCCAGCTGGGGCATATGAAGCCCGATCCTCTGACATGACTG
TCTCAACCTCTGTCTGCTTCTCAGTCAGCTTCTCCATGATGATGTCAGCCAGGGTGCGCCTAAGAGGGGAAGTGCCAAGAGGAGGGAGAGAGGGG
CAAGGAGCACTGTGGTCACTGTCCAGGAAGCTCCAGACTCCCCACAGTCCCCTGTGAGCACCGCCATACTGCGATCCTGCTGGGATGCAGCTTAG
AGTTCTCTCTCTGTGCCCATTCATCCACCACAGACAGAATCACTGCAGGGTGCAAAGGTCTGTGTCATTCCATTTAGGACTGCTAGCAGTTGTTG
TACGGAGGTTATTATGAACCTCACTCTGAGTCAGTCTGCCCAGGTTCATCCTGGCTCACCCACTCCCTGGTATTTCTTTGGGGCATTGTTACTTT
ACCTCTCTATGCCTCTGTTTCCTCATTTGTAAAATAGGATAATAACGGTACCCACCTTAAAAGATTAATGGAAGGTTAAATAAGTTAATGTCTGT
TCATTACTTGGAATGGTCAGTACACAGTAAGCGTTATCTATGGAAATATTTCTAAGACTGATTATATTCTTTTTGCCTCCTTTTCAGTATTGTTG
GGTGTATCAACATAATCTTATCTTAATGATTCAGAATTACCATCGCCTTGAGCTGTCATATAATACTAGCATCTTCAGATCTGCTGTGAAGTATT
TATTTATTTTTTTTGAGAGAGAGTCTACCTCTGTTGCCCAGGTTGGCATGCAGTGGCACCATCATGGCTTCTGTGCCTCTATCTCTCTGGGGCTT
GCGATCTTCCCGCCTCAGTCTCTCGAGTAGCTGGGACCACAGGAACATAGTACAAGGCCCAGCTAGTTTTTATTCTTCTGTAGAGATGTGTCTCC
CCATGTTGCCCAGGCTGGTCTCACACTCCTGGGCTCCTGCCTTGGCGTCCCAAAGTGCTAAGATTACAGGTCTGGGCCACTTGCCTGCTGAAAAT
GTATTTAAGCTGCTTCTCAGTTGGGCGCAGTGGCTCACGCTTGTAATCCCAGTACTTTGGGAGGCTGATGAGGTGGATTCGTGAGGTCCATACTC
TTGAGACCAGCCTGGCCAACATGGTGAAACCCTGTCTCTACTAATAAAACAAAATTAGCCAGGCGTGGTGGTATTGCCTGTTTCCTGCATACCTC
GAGAGGCTGAGGTTGCAGTGAGCTGAGATCATGCCACTGCCCTCCAGCGTGGGTGACAGAGTGAGATCCTGTCACAATCCAAAAAAAAACAAACT
GCTCCTCTTTTCTGAGGTCCTACTCAATGCCAGGCACTGAGTTTGGGACTTTTCAGTCCTAGAGTCAGCGCAACACTTATAGGACACGGNCTTAT
GCAGACATGGGAACTGGGCCTCAGGTTAATAATCACCCAAGGCCTCACAGCTGTCAGGTGGGGGTAAGAGTCAAACTCATTTTTTTTGGTGTCTG
AGTTAAGGTGCTTTATAGTACCTTATAATGTGCCCCCAAACTATTGCCTCTGATTGTTGAATTCATGTGTTCTATCTTGCTTTTTTCTGTGATGC
CAGCTATCATTTCTTACGACTGCCAGGAGTTCTGATTACTTCAAGGAGCTTGTCTACTCTATTTACTACAAGCTGGAAAGCATTCTGCTTTTTTG
TCAGCATTATCAGTGAAGTAGGAGTTATGAGGTGCTGAGTGAGAACAGAGAGGCTCTCTGGGGGGACAAGCCCTGTACATACTTTATGATTTATA
TTTCACTGTTAGGCATCAATCTGGGAGAGGGGCTCATTTTCATTTCTTCATTCTGCATTTTCAAATTGCTGCCACAGAATTTATTTGCCTCCCTG
TCCTTTTATAGTATGATTAGCAATGAGATGCATTAGCCCAAAACAGCCTAAAGTATCAGGGACACCATTATGTTAATATCACAGGTCAGAATGTC
TGGTTGAGGACCCTTATCCTAGACTCCACTCCCAAGGGCCAAAATTTATGGGCTCAGGAGTTTGACCACAGGCAGTCTAATGCCCATCATTTTGG
AGCTAGCATTTATTATAGATGGGAATAAATTAGTGGTCCTGACTAGGGCTTTCTTGCCTCCCAGAGGACATTTGCAATGTCTATGTCATTTTTTG
GTTTTCACAACTGGGGGAAGGTTGCTACTGGAGTCTAGAAGTAGAGATCAGAGATATCTACTAAACCTCCTACACTGCACAGGACAGCCTCCACA
CACACAAAATTACCCAGCCCAAAATGTCAATAATGCTGAGGTTGAAAAACCTTGGCTTAAATGAAGCTTAGGATATGAAGACTTGTCAAGAAGTT
GAGCTTACCGCTAAAAAAACTAAGAATACCAAAACCAAATGCAATAGGATGGCTCTCCCCAGAGGTGTAAAAGGGCCTGGAACTGTTCCCTGGCA
ACTGGGCAGTTGTACAACAAATGGACATAAGGACACAATGTTTTTGAGGCTGGGTCATTCCTACTCACAGTGGTCCTTTGAGGATGGCCAGTTTT
GTCAAGGTGGTCCCCTACAGCTAGCAACTCCAGGGCCCTTTCCGGGGTAGGGAATCATAAGTCTGCCAGGGAACTTGTTTTCCTGTGGGTCTTTC
TACAAGGACTCATTATCCCAAATCCCCAGGCCTACCTGGCAGGAGGGTTATGTTCATGAACATCTCTATGGCACGCTCATCCTCAGGGTCCACTC
CACCTCTGCATGATGGCCCGCTGCTGTCATTGTGGCAGCCTTCTCCAGGGTGGGCCACTCCTCGTCCTCGTCATGATCCATCCTGAGGCATTCTT
GGACCTAGAAGAGGGGAGACTAAATGGATGAGCACAGCAAAAGGTAGCTGAGGATTTCGTCAGTTTAACTTTAGCCATTATGTAGTACAGGCTCA
CATGTGGTTGTGATAGACGCAGGTATGTGCAGTGAAACAAATGCATTAGCTCCCTGTCCCCAGGGTTGCAATCATGCTTGAGAAACAAAATGTAT
ATACAGGAAACAATAAGAAAAGCAATGGCCATAAATAATAGTTAACATACCATACTCAGTTATGTAGAAGGAACTTTTTTTTTTTTTTTGAATGG
AGTCTTGCCTGTCACCCACACTGGAGTGCAGTGGAGGATCTCAGCTCACTGCTGCTCCGCCTCCCGTTTCATGCCATTCTCCTGCCTCTCAGCCT
CCCAAGCAGCTGGGACTACAGGCATAAGCCACCAGCCCGGCTAATTTTTTGTATTTTTAGTAGAGTTGGTTTTCACTTGTTGACTGAAATGGTCT
CGATCTCTTGACCTCGTGATCCGCCCCGCCTTGGCCTCCCAAAATGCTGGGATTACACGTGAGCCACTGTTCCCGGCCTGTAGAAGGAACTTCTA
AACACTATACCCATGTTAACTGTTTAATCCTTATAAACCCAAGAAGTGGCGGGCANNNNNNNNNNNNNNNNNNACGGTGGCTCACGCATGTAATC
CCAGCACTTTGGGAGGCCGAGGCGGGCAGATCACGAGGTCAAGAGATCGAAACCACCCTGGTCAACATGGTGNCCCCATCTAACTTAAAATTACA
AAAATTAGCTGGGCGTGGTGGTGGGCGCCTGTAGTCCCAGCTATTTGGAAGGCTGAGGCAGGAGAATTCTTTCCCAGGCAGGTGGAGGTTGCAGT
GAGCCAAGGTCACGCCACTGCACTCCAGCCTGGTGACAAAGCGAGACTCCGTCTCAAAAATATATATAAATTAAAAAATAAAAAATAAACCTTCT
TATGGACIATGGCAAATAATAAATAAATAAGAATAAACCCAAGAAGTGGATACTATTACTATACCCAGTTTCNTGAGGACAATTTGAAGCACTGA
GAAGTTGGGTAATTTGCCCAAGATGGTAGTCTGGGTCTAGACTGCATTCTTTCGCTATTCTACACATATTCTGTTTGATTATATGCCACAAATTG
AGTAGTAGAGACAACATATGCACTTAAAGGATGAGTGGTGAGGACAGTTAGGAGCTGGATGGGCAGTTTGGGAGCAGGGAGTGTGTTCCAGACGC
AGAGAAAATAAGGCTTGGATATAAGAGTCTCCTAGGCAATAGCAAGAAATAATGCTAGGTACGTGGGNGGTGTGCCAGACTGGAGAGGCTTTTAA
ATTCAGGCAGTAGAGTTCAGATTTCATCCACAGTCCATTTAAATACATGAAGTTGGCTTGCGTTTTTCTCACGGGCCTGTAATCCCACCATTTTG
GGAGACCAAGGCGGGTGGATCATGAGATTGGGAGTTCGAGACCAGCCTGACTAACAGGGAGAAGCCCCCGTCTCTACTAAATACAAATTAGCCGG
GCTTGGTGGCGCATGCCTATTTCCCAGCTACTTGGGAAGGCTGAGGCATGAGTTCGCTTTCCCTGAGGTGGAGGTTGAGGTGGTTAGGTGAGCCG
AGATCGCGCCACTGCACTCCAGCCTGGGCAACAACAGCAAAACTCTGTCTCAAAAATAAATAAATAAAATAAATAAAATAAATAGGCCGGGCGCG
GTGGCTCACGCCTGTAATCCCAGCACTTAGAGAGGCCAAGGCGGGCGGATCACGAGGTCAGGAGACTGAGACCATCCTTCTTATCTTTGAAACCC
CGTCTCTACTAAAAATACAAAAAATTAGCCGGGTGTGGTGGCGGGCGCCTGTAGTCCCAGCTACTCGGGAGGCTGAGGCATAGTTGGTGTGAACC
CGGGAGGCGGAGCTTGCAGTGAGCCAAGATCGTGCCACTGCACTCCAGCCTGGGCGACAGAGCGAGACTCCGTCTTAAAAAAAAAAAAAAATGAA
GTTAGTCCATTTAAATAACTTCTGTTTTGGAAGGCTACATGTCTGGGCATCTGGAAGGGGTCTAATAATCCAAGTGGGGAGCTATAATGCTACGT
ATCAATAGGTCATTAACACAAAGGCAGTGTGTGATAACACTAAATAAGGGGGAACAGACTTTTTGTCTCTCGTTCCTCGGTCCCCCCCAAATTTG
TACTTTGTAGGTGGTTCTCCTTAGTGCCAGACCCTAACTCCAGGTGTCTCCTGTAAGCTGCTCTTGGATGACTCAGGCACCTCAAATCTCACCCG
TTATCACTAAACTAATTTTCTTTCCCTCCTAGGTCTACACTGCTAACCATCCACAGCTCAGTTCGGCACCATCGTCCACCAATTGCTAAGCCCTG
GGAGG
HUMAN SEQUENCE—mRNA
GCGCCTGTCAGGGAAGCGGCGCGCGCGCGCGGGCGGCGGCGGGCTGGGATCCGCCGCGCAGTGCCAGCGCCAGCGCCCAGACCGCGCCCCGCGCT
CTCCGGCCCGTCGCCTGCCTTGGGACTCGCGAGCCCGCACTCCCGCCCTGCCTGTTCGCTGCCCGAGTATGGAGCTGCTGTGTTGCGTTTCCCCG
GCACGCGCCCCGGGCCGGGCCGGACCCGCGGCTGCTGGGGGACCAGCGTGTCCTGCAGAGCCTGCTCCGCCTGGAGGAGGAGCGCTACGTACCCG
TCCTACTTCCAGTGCGTGGGGCGGGAGATCAAGCCGCACATGCGGAAGATGCTGGCTTACTGGATGAGGATTATGTGATAGCAGCAGCTGTGAGG
AGGAAGTCTTCCCCCTGGCCATGAACTACCTGGATCGCTACCTGTCTTGCGTCCCCACCCGAAAGGCGCAGTTGTGCTCCTGTTGCGGTCTGCAT
GCTGCTGGCCTCCAAGCTGCGCGAGACCACGCCCCTGACCATCGAAAAACTGTGCATCTACACCGACCACGCTGTCTCTCCCCGCCAGTTGGGAC
TGGGAGGTGCTGGTCCTAGGGTGCTCTGTGGGACCTGGCTGCTGTGATTGCACATGATTTCCTTCTTCATTCTGCACCGGCTCTCTCTGCCCCCC
GTGACCGACAGGCCTTGGTCAAAAAGCATGCCCAGACCTTTTTGGCCCTCTGTGCTACAGATTATACCTTTGCCATGTACCCGCTCCATGATCAA
CACGGGCAGCATTGGGGCTGCAGTGCAAGGCCTGGGTGCCTGCTCCATGTCCGGGGATGAGCTCACAGAGCTGCTGGTGTATCACTTCACTGAAT
GTGGACTGCCTGCGGGCCTGTCAGGAGCAGATCGAAGCTGCACTCAGGGAGAGCCTCAGGGAAGCCTCTCAGACCAGCTCCAGCCCAGCGCTTTG
CCCCCCGGGGCTCCAGCAGCCAGGCCCAGCCAGACCAGCACTCCTACAGATGTCACAGCCATACACCTGTAGCCCTGGAGATCCCTCTTAGGGTG
GCCACTAAGCAGAGGAGGGGCCGCTGCCACCCACCTCCCTGCCTCCAGGAACCACACCACATCTAAGCCTGAGGGGCGTCTGTTCCCCCTTTTCT
AGCCCAAGGGATCTGGTCCTACCCATCCCCGCAGTGTGCACTAAGGGGCCCGGCCAGCCATGTCTGCATTTCGGTGGCTAGTCTGCTCCTCTCCC
TGCATCTGACCAGCAGCGCCTTTCCCAACTCTAGCTGGGGGTGGGCCAGGCTGATGGGACAGAATTGGATACATACACCAGCATTCCTTTGTCCC
CCCCCCCCCACCCCTGGGGGCTCTCATGTTTTCAACTGCCAAAATGCTCTAGTGCCTTCTAAAGGTGTTGTCCCTTCTAGGGGTTATTGTTGGAT
TGGGGTCCCTCTAAAATTTAATGCATGATAGACACATATGAGGGGGAATAGTCTAGATGGCTCCTCTCAGTACTTTGGAGGCCCTATGTAGTTGT
GCTGACAGCTGCTCCTAGAGGGAGGGGCCTAGGCCTCAGCCAGAGAAGCTATAAATTCCTCTTTGCTTTGCTTTCTGCTCAGCTCTCCTGTGTTT
TGACAGCTTTTGCTGCTGAAGGCTCATTTTATTTATTAATTGCTTTGAGCACAACTTAAGAGGACATAATGGGGCCTGGCCATCCACAAGTGGTG
GTAACCCTGGTGGTTGCTGTTTTCCTCCCTTCTGCTACTGGCAAAGGATCTTTGTGGCCAAGGAGCTGCTATAGCCTGGTTTGGGTCATGCCCTC
CTCTCCCATTGTCCCTCTGCCCCATCCTCCAGCAGGGAAAATGCAGCAGGGATGCCCTGGAGGTGGCTGAGCCCCTGTCTAGAGAGGAGGCTGCC
CTGTTGACACAGGTCTTTCCTAAGGCTGCAAGGTTTAGGCTGGTGGCCCAGGACCATCATCCTACTGTAATAAAGATGATTGTGTAAATTTCTTC
TTTGG
HUMAN SEQUENCE—CODING
ATGGAGCTGCTGTGTTGCGAAGGCACCCGGCACGCGCCCCGGCCGGGCCGGACCCGCGGCTGCTGGGGACCAGCGTGTCCTGCAGAGCCTGCTCC
GCCTGGAGGAGCGCTACGTACCCCGCGCCTCCTACTTCCAGTGCGTGCAGCGGGAGATCAAGCCGCACATGCGGAATGCTGGCTTACTGGATGCT
GGAGGTATGTGAGGAGCAGCGCTGTGAGGAGGAAGTCTTCCCCCTGGCCATGAACTACCTGGATCGCTACCTGTCTTGCGTCCCCACCAAAGGCG
CAGTTGCAGCTCCTGGGTGCGGTCTGCATGCTGCTGGCCTCCAAGCTGCGCGAGACCACGCCCCTGACCATCGAAACTGTGCATCTACACCGACC
ACGCTGTCTCTCCCGCCAGTTGCGGACTGGGAGGTGCTGGTCCTAGGGAAGCTCAAGTGGGACCTGGCTGCTGTGATTGCACATGATTTCCTGGC
CTTCATTCTGCACCGGCTETCTCTGCCCCGTGACCGACAGGCCTTGGTCAAAAAGCATGCCCAGACETTTTGCCCTCTGTGCTACAGATTATACC
TTTGCCATGTACCCGCCATCCATGATCGCCACGGGCAGCATTGGGGCTGCAGTGCAAGGCCGGTGCCTGCTCCATGTCCGGGGATGAGCTCACAG
AGCTGCTGGCAGGGATCACTGGCACTGAAGTGGACTGCCTGCGGGCCTGTCAGGAGCAGATCGAAGCTGCACTCAGGGAGAGCCTCAGGAGCCTC
TCAGACCAGCTCCAGCCCAGCGCCCAAAGCCCCCCGGGGCTCCAGCAGCCAAGGGCCCAGCCAGACCAGCACTCCTACAGATGTCAGCCATACAC
CTGTAG

TABLE VII
MOUSE NOMENCLATURE
ICSGNM Wnt3
Celera mCG19162
HUMAN NOMENCLATURE
HGNC WNT3
Calera hCG27972
MOUSE SEQUENCE—GENOMIC
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNMNNNNNNNNNNNNNNNNNNNNNNNNMNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NMNNNNNNNNNNNNNNNNNNNNNNNNNNNNNNNNMNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNGGCTGCCAGCAACCAGGCCCTTCT
GGGGCCAGTTCCCTCCGGGCAGATAGATCAGCTGGCCCCAGGGGTGCAGGATCCGAGGGGTCAAGCCCCCAGAGTGGCAGCTTTGTGTGATGGAATG
AGGCTTAAACTCCAGTTGGCAACCTTCAGTACATTCATGGGGGCAGCCACCACTGTGTGCACACAAACAAGCACCACTGTGCGCACACAAGCAAGCA
CCTCTGTGTGCACACGAGCAAGCACCACTGTGTGCACACAAGCCAGCACCACTGTGTGCACACAAGCAAGCACCACTGTATGCACACTAGCATCACT
GTGTGCACACAAGCCAGCACCACTGTATGCACACAAGCCAGCACCACTGTGTCACACGAGCACCACTGTATGCACACAAGCCAGCACCACCTGTGTG
CAACCAAGCAAGCACCACTGTGTACAAACAAGCACCACTGTGTGCACCCAAGCAAGCACCACTGTGTGCACACAAGCAAGCACCACTGTGTGCACAC
AAGCATCACTGTGTGCACACAAGCAAOCACCACTGTGTACAAACAAGCACCACTGTGTGCACACAAGCATCACTGTGTGCACACAAGCAAGCACCAC
TGTGTACAAACAAGCACCGCTCTGTGCACACAAGCAAGCACCACTGTGTACAAACAAGCACCGOTCTGTGCACACAAGCAAGCACCACTGTGTGCAC
ACAAGCAAGCACCACTGTGTGCACACAGGCACATGTGCATGCTGCACCCCAGCTTTCTCTTACCCACCTCCTTCTGGGACCCTGAAATCTCTGTGAC
TGCTTGGAACATGCTAAGAGGTCCATGGACACCACCCAGTCCTGTGAGTAGTAAGTACCTCCTGGGCTAGATATGACCCTCACCTATAGCAAGTGTC
CGCAGAAOGCTGAGGTAGCCACTTCTAGGGACCCACAGCTGAGTTCCTGTTCAGTGCCCTGGTCTGTCCTGCAAGGTAAGGACATCAAGGATAATGA
CCATTAGTAATGGTGGAGAAACGAAGCATCTAGTCTAGTTAGCTGTCACOGCCAAAAGGACTCTTAATTAGGCCACGTTAATCTATAGGAAGGGTCA
TAGTCAAAGAG0GTAAGTGAGCATGCAGCCCAACGACAGAAAATCGGCCAGGGACCCCTTGGCTCCCCACCCACATCCTCCCAGCTCTCACATTCTG
ATGGAGGACAACAGGTAGAGACTAACAGCTTTTCTGGGCCCTGGACTGTCTACCTTGTCTGAAGTCTACAGTGTCTGAAOTAGGTATCTGAACTTGA
CCCAGCCCTCTTCCTCGCCTTCCTCCTCCCACTCCTGAAGAAAGCTTCTCACAGACCAAAGAAGTGATCACTGGCATCGCCAGGCTCCTCTGGCAGC
TGAGAGGCCGTCACGCTTCCTCCTTACTGGGAATATCTGGGTCGCAGAGGCCTCCGTATGACCCATACTTTCACCCCACTTACTCGTAAGGCTGGAT
TGGTGTCCCCAAATATGTCCAATACTTTGAGACGGGGTGGGGGTGGGGGGTCATGGCCATGGAGATTCCAGATGCAGAGGAGGGAACTCAGTGCCAC
CTTTGGCTGTAAGGAACAGCCTCAATTGGGATTAGGAAAATACACCATCTTGAATCATAAACAAACAAACAAACAAACAAATAAATAAATAATCTTG
AATCACAAACAAACAAACATTCCAAGACGGGATTCTCAGGGCAGTTAATGTCTCAGCAGTGGCTCAAGAGACTCAGTTTCTCCCACCCTGGTCTTAA
GTCTCTACTTTGGTCTCAGTCAGTTGTTCTCACAGCATCCAACCAAGATATAACAATACCCCAAAGCGGGGAAACTGGACAGCAATTTAGCGGACTG
CTCACTCCCCTAAATACTTTCTAGAAATCACAGGTCCGTCAGCTGACTCCCCTTCGTGTCTCCTTTGAGTCGGTGTAGCCCAAGCCGTTGCAAATTG
ACTCAGACTAGTCAGCATTTATCTCTGAGCCAGGTGGGGGAGAACAAACTTGGTTCCTCATGTGCCTAAATAAAGGGTCTGCCTTTACATATACTTT
TATATACATATATGTGTATATATATCATCCTAGTCAGGATTTCTACTGCTGCACAAAACATCATGACCAAGAAACAAATGGGGGAGGAAAGGGTTTA
TTTATCTTATACTTCCACATTGCTGTTCATCCCCAAAGGAAGTCAGGACTGGAACTCACACAGGGGAGGAAGCAGGAGGCAGGAGCTGATGCAGAGG
CCATGGAGGGTGCTGCTTACTGGATTGCATCTCCTAGCTTGCTCAGCTTGCTTTCTTATAGAACCTGGGACTGCCAGCCCAGGGATGGCACCCCCCA
CAGTGGGCCCTCCCAGCCTTGATCACTAATTAAGAAAATGCCTTATAGCTGGATCTGATGGAGGCATTTCCTCAAGGGAGGCTCCTTTCTCTGTGAG
AACTCCAGCTTGTGTCAGGTGGACACAAAACCAGCCAGTACAGTTACTTGCAACCATAAGTAACTCCAGTTCCAGGGATCCTGACCTCTGCAGGCAT
CGAGCAGGTGGATGGCATACATGCATACAGGCAAAACACTCGTACGTGTAAAAGAAGATAAATCCALAATAGTAATAATAATAAAAAGTAACTCAAA
AGAACTTTCCTCCTCACTCAGCTCAGCTACTGCCAGGAACCATTTCAGAGCCAGCAGGCATCACCACTGCAGGCGAGGGAATAGGCTTAGAGAGCAT
AGGAAACATGAAACATGCCCAGGGTTGTACAGTACGAGGCAGAGCTGGGGTCTAAGGCAACCACTCAGACAGTGTCTCCCTGGACAAACAACCGAAA
TCCCATAGGACAGATCCTGAGAGGCCAGCAATGCTAAGCAGGGTTTGCCCAAGGACCGCCCCCCACCACCTGTGACAGCTATAAACACCTGTTCCAG
GATCTTTGCTGGTGAAGTAGCGAGGGCCCCAGCGACTGTCCACTGAACCATCTACTTGTGTTCAGACTCTTAGGGGGTAGAGCTCATTTCTGGTCCA
ACCTCAGTTTCCCCACGGTGGCTGCTGTTGGTCATACTTGTCTGTGAAGGGATGGTTGGGGGCAGAGATCTGGAGGGTGCAAACCAAGCATGATGGG
CAATTGTCTGCCCTATATCCATGGATCAGACCAGGCTACTCGATCCCCCCCCCCTTTTTTTTAAATAGTGTCCCCACURGACTCTTCTCCCAAGAGT
CTGGCTGGCTCTTAATTTTGCTGTTTAGAAACATTTGATTTTCTGAGGTATGCCTCTTCCAGGAGAAGGTAAGCCTCATAGACTCAGAACCCTGGGG
CTCAGGCATGCTTACCTGGGGGTTACCTGCATATAGTCCTTCTTAGTGAACAACTAACTACACAGGACACCTATGCAAAGAACACCTGGGAGCTCTG
TAATGGCATGCACATGTGTGCACACCACACACACACACACACACACACACACACAGAGAGAGAGAGAGAGAGAGAGAGAGAGAGAGAGAGAGAGAGA
GAGCGATCATTGTGGCTGGAGGGGCTGTCAAGTCTCAGATGCCTCCAGGTGTCAGTGACTTCTGGCCAGGTTGCTATGTTCTCATCTCAAGCCATCA
GGAAAGGTGGGTGTTACCTTGAGGAAAACTGAACCCAACACTGGGGTGGACAGGTCTCCAGGAAGCATTAAAGTGCCTTAAAATACCACACGGAACA
TAAACCCAGGGCCCTCTCATCTGGGATCTCAACACCCTCTCTAAGTCTGTCAGCCTACTCTGTTCTTGGTGCCAGAGTTCTCCTGAGTTCAGTTGGA
TAAAGGGCAGCACACAGCACACAGGCTTAGGTGTGCCATGTGTTAACTCCATGGCAGTGTTAGGGGCTTCTGGCCAGAGCCTAGGGTAGGGGTTGGC
CACTGATGGAGGTGGAGAGACTCTCCAAAGAGATGCTGATGTGGGAGTCCTACAAGGGGCGGGGGCGGGGTGGGTGGGTTGGCCTCTGATACTAGGA
CATGTTAGGGGGAGAGGAGTGCCCTTGTCACAGAAAAAGAAGAAAGCCAGTGTTTTTTTGTTTGTTTGTTTGTTTGTTTTTTAACCATCCCAAGTCA
GCTTTGTGTAGTATTAATGTCTAATGATAAGGACTCCCTGTGAGATAGCTAAGTCCCATTCTCACACCACAATTGAGGGTACATTCAGGGAGCAGCT
ACCTCCACTGGCCTTCATTTCTTCTTCCAAAGGACCCCTTCCAAATTACCCTGGGGACCTGCCGCTGCTGTTGGAGGATCACTGGAAAAAGGGGACA
CCTGACATCTTCAGGTCTGGCAGACCTAGACCTAGAAAGGATACCTGTTCTGGGTGGAAGGCCTTGGGGTATCACAGGTCCACTCCCAGAGATGCCA
ATCTGTATCCCATAAGCCAGTCTGCTGACTGTCTCTAGGGCCTGCAAGTCCTCCCAGGTGACTCAGGGGTGCCTTGAGCCTCTAAGTGGAGCACACT
TGATCCCCAGGTAAGCTCCGAAATTGCACAGTAGATTAAGGGAATTCACTCACAAGGATGCCTCCTTAGCTGAGATCCCCAAAGATCCAGGCTGCAT
AGGACCTGAAGGCTCACTCACTTCTGCTTTCCAGTCAGCAGCAATGTAGCAATGTAGGAGCCAGGCAGCACAGGGAAGGCTCTGAGCTCTGGGACAG
ACACCTGCATCTCTTTGGGTGCTTGGATCCCCCTTCTCTTTGGCCTTTCTGCAGGCCACAGGCTTCGACTTCTGAGTCTTCACTTAGGATGATGAGG
CCTACCTGCATGAATAGCGTCGTGTCTAATTTTTGTCTCCGCCAACCCCTCCATGATCCTCCTACTTCCAAGGCCACCTGCTGTCTGGTCTCCTAGC
TGTCTCCAGGAAACTCACCCGAAATCGTTCGAGAGGCTCCTAGAGTTGTTAAGGGAGACCCTTAAAAGCTCCCTTCTCCTGGTCAGAGACCTTGGAG
TCTCCCGTTCCTTCTGGTGTTCTTCTGAGAATCTCTGCTTCAGGTCCCTTGACCTTCGCACCTGATGCCTCCCTGCTTTGCCCACAGTGGTTTTTCT
CCCTCCATTCCTACATTAGGCTAGGTTGGTTCTGAGCCCCCACCCCCATCCCGCAGTTCCCAGAGGGAACAGTCCCAGCAAAAATACATGGAACTCA
GTGCCTGAGTGGGAGATTTGCCCCTCCCTCCTCACGACAGACTGGCAGCCGAGTGAGGGCGGGTGGGGGTCTAGCGGCCAAATTCTTTGTCTTCGAG
GTGCCTCAGCAGGGGTCCGGTGGAGCAGCGCAGAATACACACAGCCGCTGACCGATAACTCCTGGAGAGAGCGCACTTCAAAAGGAACCTCCCGGCA
GCTACTGAGGTGGGGGTGGGGGTCCCCATCTCGGGTCTGAAGTCCCTGTCAGTCACACACAGCACGCAGGCTTCCACGTGCCACATCTTTTGCTGCA
GGTCCTGGGTCCCAGATGCCTTCCCCTCCCTCAACCAGGAGACCACCCTTCCTGTCCAGGGCGAGCCTCATATCCACCCTCATTAGCTGGTTTCAGA
GTAAAAATAATCCTTTCCCGCCATGTGCAGCCTACCCTGATGTTGGGTGCCTATCAAGAGAGAAGATGTGTCTAGGAGATTCTCGTGGCAAAGGGGC
TCCCTCACTGCCTGGGCAGGGTCCATTCCTGCGTTGCCACCCGCAGGCAGGAGGAAGAAAATTAAGCCTGTGTTGAAGGAAGGACACCTGGCAAGAG
AGACGAAAGGACTCCGGAATCCCCTCCTCTGGGGAACTTTCAAGTGGGGGGGAAATCCCCACGCAGGCTTCTACTCTGCGGCAGCAACATGCGCGGC
CACTCCCCGCTTCTGGTCCCCTCCTCCTTTCCTTCTTGCACCTTTGCCTGTTTGTTTCCTACCCTCAGAACAGCACCTGGGGCTGCCTCGGGCTCTG
GTTTGCCTGTAAGAGGCACCTGCATACACACAGCCCTTCTAGGGACGACCAGTTTTAGCCACACCAGGACAGCTTCCCACGTATCTCGCAGGCAGCC
AACATCTCACCCAAAGCCACCAAGAGCCTTTGCCCCCGCTCTCTTGGGCTGGCACCTGTGGAATCTTAAGGATCTAAATGACCCCAAGAAGCCCTCC
AGATTAGCTCTTGCCTCCACACCATAGCGGTGTGTTGGAGGCCAGAGAAAGCAACGTCCCTGTTTTCAGAAACACTAATTTTGGACTGCCCCCCCCC
CCGCCCCACGCTCTGATGCAGGACTAGAGATTTAATCCGCATAGATGTTCAGCGGCCTATGGGGAGCCCCCATGGGATGAGCAGCAATCAAGATCTC
AGCTAAAGGCTACCTAGTAACCCAAAAGCCTGTGAAGCAAGCTCCTTGACAGAAGCCTCAGCCCCGGGGCTTTGCATTCCCCGATAGTGGCCAACCC
CCTCCTACTGCCCTCCCAGGTGAATCAGAAACGGGGCCTCAGAATCAGCCGTGCCCCTTGGGGAGGAATCTTCTGGGAATGCCCGGCCCCCGGGGGC
ATTTCTAGGGGTGCTTATCTCAGTACCATCTACCAGCTCCACTAGTCTTCAGCAGGTGGGGGATTGCCTGCTGTTCCTTAGCCCTCCCCACTTCTCC
CCTTCCCCTTTCAACCCCCACCTCTTCCTCTGTCTGGGCGTTCACCCCACCCCCGCCCCTGTTTTCCGTCTCAGTGTCTCTATCCACTTGTCTCTCT
CTATCTCCCCTTCCTCCCCTCTTTCATCTCTCCCTCTACCCCCCTCCAGCCGAGGATCGGGCTAGGAACTCGCAGAAGCTGTGACAAGCCCAGGCGG
CTCTCTCGACCCTTGGTGCCCCCAGGGGCCAGTTCCTTGCTCTCCTTACACCAGCAGAAGCCATGTGCCGAGAACAGACCAGCAGGGGCAGTGGATC
CGAGCTGTGGGCACCAGCCTGCCGGATCAAAGGCTAGGAATGTCTGCTCCCCGTAGGACTTCCCTCAGTCCCATCCACCCCTAACAGCAAGAAAGCC
CGCCATTCCTGGGTAGGCAGGTTTGGGTGGGCTCCCTCTCTTCACCGTCTACACAGTTAAGTCCCACCATGGCTTCTCCTTTGGCTTGCTTTCTAGG
TCCCAAGCCAGGAGCATTTCAAGGGCTGAATGTCTTCTGTGTCCGGGTAGAGAGGCTGAGGTACCTAGGTAAGGGGACTGAATCCTAACAATCTAGG
TATGATTGGGCTCAGACTTAACTCCCAAACTGTGGAGAACTGGTAAGAAGAGTGCCCCTTGGAGGCCTGAACAGTTCCCCTTGTATAAGAGCTTCAG
TAGGTCGTAGGGTGAATCTGGTGTCTGCACGGGACAACGCTTAAACACCAATCGCCAGGTTTTAGTTTCTCATCCGTAGAAGGGTCGGGAATATAGG
TCAGAGACCTGGACATAGGGGAGGTAGATGTTCCTGAAGAGCCCTCGCCCCCAACCACTAATAGACAGAGCTATACCGTGTTTCACTAGTCCCAAAT
GCTTCCATCCTAAGATCCAAGAAACTGCAGGTACTGCTGAGGTCCGGCCAGCACGCCTGCAGGAGGAGAAAGTGACCGAATCCTCCCGCAGGAAATG
CAAACGGCGGCTGGTGTCAGTTGCAGGAAATGCAAAGGCTGCAGGAGGTCGCGATACCAATCTGACTCTTGACAAAAGACTCAAATGACACTAGCTC
TGTCGCCCGGCGCCGCCGGCTGGCAGACCTTCAGAGCCTCAGCGGACTCTCCCGCGCGCCCCTCGGACACCCTGGAAGCGACCTGCGGCCGGGTGGC
AGGGGCCGCCAGCTCGCGAGCCCGAAAAGGCTGGAGACTGGGAGGGGTTGCATTTTCCTAGCCCCTGATCCCAGTCGCGCGATCTGGAAACAACCCC
TTTGCTTTCTAGTCTCGATTCCTTACGGGCCCAAGTGCCAAGGGATCACAGAAAAGGGAGCCACCCATCAATGCCCAAGCCGACCCCTTCACCACGT
GAACGCCGGGTCAAAGCTCCAGCTCCCTAAACCTGGCTCGCGTGGACTGGGAAGGGGGTGTCCCAGGCCAGCAGAGAATCCAGCGTGCCGGGTCCTC
CCGAGGCCTCCGTGCGCCCGGGGCGCAGGCTTTGCCCAAGTTTGCCGGGAGATGCCCTTCCCAGAGCCGGAGTTGTGGGCCGGGCGGGAGGGGCCCG
TGATTGACAGGCTGAACTACAGACTCATCTCTTACCTTAGGCCGCGGGCGCTGATTGGCTGCTCGCTGACATCCTCAAACCCGGCTGCTCGGCGCTG
GGCTCCGGAGGGGGGCGGCTGCGGGTGGAGGTGCGCCTCTGACAAGCCCGAAAGTCATTTCCAACCTCAAGTGGACTTTGTTCCAACTATTGGGGGC
GTCGCTCCCCCTCTTCATGATCGCCGGCAAACTTCCTCCTCGGCGCTGCTTCTAATGGAGCCCCACCTGCTCGGGCTGCTACTCGGCCTCCTGCTCA
GTGGCACCAGGGTCCTCGCTGGCTACCCAATTTGGTGGTAAGACTTGCCTCTTGTCTGCCTGCGGCCCCGATCTCTCGGCCGCACCCGGGGACGGTG
GGAGGCGCTGGCCGGGTCTGGGCTGCAGCAGGCCTGGGGCTCTTTTCTCCTTGCTTCCCGCTAGCCCCCCAGCCGTCCACCCCTCTTGGATCTTCTG
CTGGTGTCGCCTTCCGAATCCAACTCCTGGCTTCTCAGATAATCACCATTTTCCCTGCTACCCGCCGCAGAGGCGGGAGGGCTCTCCCTACCCCTGA
AGGTCTCTCGTTCTTAGTCTTCACCCTCTCCCACCCCAATTTGGGGGTCCCGAAGAACCTTCGTTTGCTCACACTTGCATTTCTGTGCTGTCCCCGC
GGCCCGGCCACTCCTCTTCTCCATTTTAGGGTCTCCGTGGGTAAAATTATCCTGTCCTTTTTCGCCACCGCACCTCGTCGGTGTCTCGGTCAAGAGA
TTTCCCCCACAATACTCGCGTTTGCCTTCATATTTGAGACCTTAAGGGCTTTGGGAATCCCAAGCAGGGGACTTAGGGGGTATTTCAGTAGATTCTC
CCCACTCCGTTCTAGATTAATTTTGAGCCTCTGAAGTGACTATAGAATTTGCTGATCCTAGATGCCATTGCTGATCCGGGTGTATTCGTTCCTTGCT
TGTCCATCCCGGAGAAAAGCGAGGTGGGGGCTCTTTGAAAATGTACTTGTTTGGGAGGGGTAGGCTTGCTGGGTTTTTTTGAGCGAAAACCTAGTTC
TCCATGTCAAATTCGTTGGTTTTGGAAGACGGGGAAGCTAGGAGAAAGGGCGAGGGAAGGGGGAATGCTGTCCCTGAGATATACGGATACAAAAGGC
ATTTTAGTAAATAAGGCTGGGCGAGCAGGCGGAGTACTTGGAGCTGATGGGTGTGTGAAGGGATTCACTGGAAGATAAACCGCCTTTTGGAAGCCCC
CCAGCCCTCTCAGTCTCCAGCCCCCTGCATTCCGGAAAACTTTTATAAAAGAACCCTCCCCTCCTGGGCAGTGGCTAGAGCCCTGGGGGGGGTGGGA
GGTGAGGAGGAGGAGGAATTTGGGACCTCTGAGTAAGAAGGAGACTGGCCCCTGGGGTGAAAGTTAGAAGTGCTACCAGGGTAAAGTGCTGCCTACA
TGGCCCCCTCCCTGCCTGCTACCTGCAAGCCTGCAGTATGAGGCTCAGAGCTTATAAAATACTGGGCCACACCCCAAGCCTTTTTCCAGATGCTTTG
GGGTGTCTGAAGGCCAGGGGAGGGGGCAGTCATGAACCCAGGCACCCAAGCCATGGCCTGCCCAAGGTTCTGTTTAGAGGGTCCAGGAGTTGCTCCT
TAGGTTGATAAATACGCCAAGGACAGACCATCTTTCTTTCCTGTGGACTTGGGCTGGTCTGGGGCTCCTGTGTGGAGTCTATAGAGCCTGTGCATGC
TGGGGACAGCCAGGGATTCCACAGTGAGGTTCTGCCAATTGGTGAAAGGTTGCCTCTGCCTCCGGACTTTCCTTCCCCCAGCCAAGGCCAAACTCTG
CCTTCTCAGAGACCCTGCGGTGGCGGGTGCATAGGAAGAGAGGGTGCCTGGAGGCCTAGGGATGGCATAGAGGAAAAAGGAGAGAGTGGGCTTGCTA
GTTTTGTGTGAAACCTTTTGGTGTGTGCCTAGGGGATCTGCGGCCCACAGCAGGGTATGCTGTATCTGCATACAAGTATCAGCAAGTGGCTGGTTCT
AGGTAAGGTCCCCATCCGACAAGAAATATTAGGAAAACCTGGAGGGCAGAGACAGGTCCTAACATAACATACATGGCCTGTTTGGGGGTTAAGGGGG
GACCAGATGGCTGCTTTTGTCTGAGCCATAGAGAGTATGTATGGGTATTTTTGAGATGGGTGGTTTGTCAGTGCCTGTGGCTACGACCTCTGTGTGT
GTAATACAGTGTGTGCTCTAGGTCAGTGACCCTAAGTGTCTGTGTACCTGTATGTCTCTGTGGGTAGAAGCTGTTAGTGCCTGAGACCCAGGCAAGT
GTTTTGTGTGAAACCTTTTGGTGTGTGCCTAGGGGATCTGCGGCCCACAGCAGGGTATGCTGTATCTGCATACAAGTATCAGCAAGTGGCTGGTTCT
GTGATGTGACACAGAAGTCGAGAAGTATCCGTTGCTTGTGTAAAGGGGGACCTCCTGGCTTTTGCCTGTGATACTGGACATGGCAGTATTTGTGTTT
GGACTTGTGTGCTGTCTGTGCATGTGTCTGTGTGTGTCCCTTCACCCGCGCATGCCTTCCTGACAGCGTAAGGGGGAGGCCCAACAAATGTCTTTGT
GCGTGACATCGGTGTTCGTGGCATGACCAGCCATGGTTGGAAGTATGAGAGTGAGCCTGGGAGGCCTGGCTGAGGGCTGTGTCCGGGCCCAGCTTCT
GATCGTGACCCCAAATGCCAGTGACATAGTTTTGTGCCAGTGGTTATGCGTCTGTGGTCATGTTTGCTGACACCTGACTTGTGGGTGTGAAGAATAC
CTGAAAGGGGTGCTTGGTGGCCATCAGTGGCCCTGAGGGGAAAGACAGGCAGGAAGTGGAGGTCTCAGAAGGTGGCTGCAGACACAATCGGTCTGGG
ATCCCAGCTGGGGTGGGGGCAGGGCCTTCCTTGACACCATCAAGCCTTGCTTGCTTGAGGACTGTGGCTACGAGCCTTCCCTCCTCTAAGGTCATTT
CCCTTTCGTGTTGGAGCTGGGGCATGAATCACCTTCATGGGGCTGGAAGTAGAGAGTGGGTAGGCCTCAAATTCTTGATTCCCCTCCTCTGCCCCAA
GGCCGGCAGATTCCAGGCGTTTGAGTCCGTGACTGCCTGCCACCTCTTTGGCCCACCCGGCTCTCACGTTCACCCAGAGGATTTACAGGGGAAATGG
CTTAGCTGGGATTTCTGTCCACATCACCTTCAGAAAGGACCCCTACTCCCAGTAGCCGATCCCTGCTTTTCCTTGCAAGGGCCCCAACTCTGAGACA
AGGTGATGAGGAAGGGGCTCTGAGGGGCCTCTTACTCTAAAAGGAAGCCCTGTGCTCCAGAGCCTCCCAGCCTGGCAGTGGCTCCATTGGAGCAGGA
CCAGTGACAACCAGCCTGCTTTCTAGAACTCTCCTTCATAGTACTAGTTAGCTCCTAGTAAAGAAATCTGTCTTTCTGTTTCTTGAGGAATAAGATC
GTTCTACGGCCTTGCCCTGTGCCATCTGTGCTCCATTGGAAGTGCCCTGAAAAAGGAGGCCTGGTTGGCTGGGTGGTGTTTGGCAGCTCACTTCACA
CTGTGTGGCTGAGGACTGGTGATTCCTCCCCACCCCCCCTTTTGGGGGTCACTTTTCTCACTTTTTACCCAACCTTGCAGTTTAGAGGGGAAATGTT
TTGCTGAGGGCTCCAAGCCTGTTAGGGCGCCTGGGTTCTCATTGCACGACTCCAGAGCCCATCCTCCACCCAAGCACCTATCTTAAGCCCTGCCTCA
GTTTCTCTATGTGTCACATCCTGCTGCCTCCCCTGCTCAACGCCCACCCAACAGCCTTCTCTCCGAGGCTTGGGAACTAAGGAACGTGTTCAGTCAA
ACCCCCGTGGGGGTCAGTGGGCTGGGCCCAGGGAGGCACCTGGAGACTGTGGGGGAGGAAGGCCCCAGGCCAGGTGTTAACACTTGTAACAGGCAGG
AATTAGAGCTGGCTGGGGCTAGGGCTGGTGAGCCGAGCTGGCTGGAGGCTGTTGATCCCACAGACAGTCAGTCAGTCAGACAGACAGACAGACAGAC
AGACAGACAGATGGGAGATACCAGATCGGATGGCCAAGCTTTAGCAGGGTGGTCGCTCTTCCAGAGAAGATCTTGCCGTGGGGGAATTCGTCTGGGG
GGAAAAAAGAATGATGGGCCCCTTGAAAATGAGTTCACAGCCCACCCCTATTGTGGGCGGGGCCTTGTGCTACAAAGGGATCGCAATTTGGTGGTTG
GTGGTAGAAGGTTGGTATCCTTCTAATGGCATCACACATTGCCACACAAACAACAAACAAGACAACTGGTGAAACACCAGCTAGTATCAGGAACTGT
GTGTGTCCTTTAGAAAAGCGGACTGGACTTTATAGGGAAGAAGGCACGTGGTTGGGGTTGGGATAGTGGTTGAGGGGGGACACAGCTGTCAGAGGAA
AGTCCTCCCCTGCCCCCTCCCCCCTTACAATCTCCAAGTGTCTTATTAACAGCTTTGGGACCAAGAGGTAAAAGCTGGGTGTGGCCCACTTCTTAAC
ATAGATTCTGAGGTCTTGGAGTTTTGGATGTCTAAACTGCATTGAATCCTGGTGTCAGAAGAGGTGACCTGGGAGCCTAGGCCTTCACAAAGCTGGT
ACCTGCTGCTGCTACCCCACTGTAAACTCAGCTAGAGCTCTCTTGGGCCCACGCTCACTCACTTCTGGGCCTCTGCTCCAACCTCCATTAAAGAGTC
AAAACGAGGGCCCCAGGTAAGAGTCCCCAAGGGACAGACTGGTCCAAGGAAGATTTCCCCACCCTCTCAGGGCAGAACTCACTGCTCACAGGAGGAC
ACAGTTGCCTGGAAGGTTAGAGCAGTATTCATGGGCCTCCTACCAAAACCGCGAAGAGAAGGCCATACCCTACATTTACCCAGCATCCCCCTGTTTT
GTTTTGTTTTGTTTTGTTTTGPTTTGTTTTGTTTTGTTTTGTTGAGATAGGATTTCTTCATGTAGCCTTGGCTGTCCTAGAACTTATTCTGTAGACC
TGACTGGCCTTGAACTCAGAGATCCTTCTTCCTGCTTCTGCCTCCTGGGTGCCGGGATCAAAGGCACCTGGCCAGCACCCTTCTTGAAGGCTCGGCC
ACCACATGTTCTGTTTCTATATATTCCCTCTGGGGAGAAATGTTTCAGCCTGGAAGTGGAGAGCCACGTCTGAGTTCTGAGCTTACCATCCATTGGC
TGTGTGACCTAGGGTGACTCTGTCCTTCTTCAGCTCCCTGCCTCTCTCTTGACTATACTGAGAGGTGTCCATTCATCTTCTTAGAGCCTCGGGGATA
TGATGGGGACTCCACAGCAGATACTTGGGACCTGCCTGAATCAGCACACCCCTTGATTTCTGTGGCTTGGTGTACCTGCCCTAAGCCCCACTTTCCA
CCCAACACTGCCATGCCTTCTTAATTCACTTCCAAACCTCACGTAAGGGCTCTCTATTCCTCTGTCTCTGGGCTGCTGGCAAAGTGACCTCTCCCCG
TCTCAGTTTCCCCATAGGAAAAATAAAGTTATGGCCGCTACCCCATGAACATCAGTGATCAAATTCAGGCCCCAGAATGCTGCCGGAATGCAGGAAG
TACAGTTCAATGTTTGAACCTCGGTCTGGCCCTGAATCCAGACTTGTCTCTTCAAATAAACCCTCCGAGATTGGCCCCCATGGCTCTCATTTCTTCT
TGTTCCATGTGCTTGGCACTGGTGCCCACAGGCTTGTAACTTCCTTCAAAATCAGAAGTTCAGCTTTTTAAAATATTTTTTATTATATTCATGTTAG
TGGTTTCCTACATGTATGCCTATGCAAGAGAGTCGGATCCCCTGGAATTAAAATTATAGACAGTTGTGAGCTGCCATGTGGGTGCTGGGAATTGAAC
CGTGAACCTCTGAAAGGACCAGTGCTCTTCACCACTGAGCCATCTCTCCAGCCCATTGAAGTTCAGCTTTTTTTTTTTTTTTTTTTTTTTTTTGCTG
CCTAGGGGAGGTAGGGTAAGGTGGACAGAGGTGGTCTGGGGTCTCCCCTAATCCTCCTGATACCCCACAGTCTTCTGAGCTCGGAAACCTCTGTCCT
CTCCAGCTGGAACACAGAGCTCCCGGGCCTGGTGGGGAGGGGTAGGCTTGGGGCCCTGGCTGCTGCAGCAGCCCCAGCACGAGGTGAAGTTGCTTCT
GGAGCTGGGGGATGACTCAGAGGGTGATTTATAGACGGAAAGCAGAAAGCTTGGCTCCTGGCAGAAGCCTGCTGCTTAATTACATTCTTTGGGGGGT
GGAGGTATGTGGGGCGACAGCAAGGGAGAGACTTCTACCTCGTCCCCTCCGGGCCTGGTACCTGTGATGTCCTAGCACCATTTAGGGTTCTTCTTAG
AGAGTGCGTCCCCTGTACTGAATGCACGGTTAGCCACTGAGAAGAGGAAGAAGTGGGACAGAATCATCGGAAGGACAGAGTCCCGTGGGGACCACCC
CAGGGACAGGCTGATGGGGCACACAGGAAGAGTGGGATGCTGAGAAGGATCGCTGAACCTCCACCCTGCTTCTGCCGAAGAATCCCTAAGCCACCTG
GGCCTCTGGGTAGCCACTCTCCACTTCCCCTTCGCGTGGTTGCCTGTGAAGACCTTCAAGTTGGCTTCTTGTCCTGTGGCTTGGGTCCTGGTATCCC
ATGCGTGCAGGAGACCAGGCCTGGAACTCAGAGACTAAACTCTGCGTCTTTGGGTTAGATGACCTGTTGCGTGGAAGTGTGATGACTGCTGCAAAAC
TGCAGCGGTTCTGGTAGCATCAGCCTCGTGCTGGAGGGAAGGAACCCCACTAACCCTGCCTTTAAACTAACTACAGCTAGCCTGTCTGCCCATGCTG
ACCAGTGTAGTGACTCCAACACAGCCCCAGATACTGAATTTCTGGGTCAGGGGTCCCAAAAGGTATAGGACAGGCAGGGGTAAGGAGGCCAAGGTCT
GCAGCACCTGGCTGCCTGTTTGAGCTTGGTTAGTACAGCTGGCAGGACTTGGCAGGGGTCAGGATGGGGACAGCAGACAGCTCTCCAGTTCCCCGTG
AGGCAGGCCTTCTCAAAATGGCCTCATTAATGCATGTGGAGTGAATTCCAGAAATGTCAGGTATACTCACACATCTAAAGAAGGATGGGGACCCAGA
AATGTTCTGTGCTTGGAGACAAGAGGAGTCAGAGTGAGGGATGGTTTCCAGGTCCGGGATCTGAACTGTTGGACATGGTCTTCCTATTGCAAAGTGA
TGCTCTGTGCTCAGGACAGTGGATGTTTGAGTTTTCTGTGCCTAGTGGGAGACTGGAACTGATTCCGGGACAGAGTAGAGACCATAAACTTTATATG
TGAGCAGCTTTGAGGTGCAAGCTGAATTGCCAACCTTCCCAGACCTCAGTTTTCTCATCCACCAAATGGCCAATAACATGCCTTCCCTGCCCATGGT
ACTAATGAGAGCTCAGGATGTCTGTCTTACCAGAGTCAGCTCAGCAACTACCATTGTTAGAAAGAAGACCAAGATCCCCACCCCCTACCCAGAGGAG
GTGCAAGAGTATCTGTAGTGGATCCCCAATCTCTGAGTAGAATCTCTGGGAGCAGAGCCGTTGCTTGGGTTTAAGACCCAGTGATGGCAGGTGTGCT
CCAGTTTCAGCTCTGGAGGTCCACGAAGAGCACATAGGCCTGATCCCCACTGAGACCCGGTTAGGAGGTCTGGGTGGAGGTGAGGGCAAGGTAGGCA
GCTGGAAGCACTCTCTCAAAGCCCGAGGCCATCAGGAAATCCAAACACCCCCAGGGAATGCCAAGGAAACCCACAGCGGCATAGCCCTGTTCCCTAA
CTGAGCTAGAGAGACTTGAAGAGAATGTCCTGTGTCCCTGTGTTGGCAAGGGGGATGGGAGCAACAGGACCCTGTGACCAGGTTTGTTGGGAGGGCC
TTTCTGCCAAACCTGCCCCTGGGCGCAGAGTCCCACTTATCTATCGGAGCCAGCCGGGATCAGCAAGGAAAACCAATCTTCCAGAGCCCGCTATGCA
TGCAAATGGGGGCTTGTGACCATGTGAACCTGCACACCGCCTCTCTCTAGTTCCTCCACACTTCATAACGTGGCTTTCCGAGGGTGTCATTCTGTCC
CTCTGAAACCTCTCATGGTTCTGATCTAGACCCCCTTTGTTTTATTTTGGCCTCTACCTGCCTATACAACATGCCTTTCAGAACCCCTCTCCCACTC
CTTCCTCAAACCGAACACAAAATAGTACATGAACCTCTGCATGCCTCCTTCCTCTGACCTGGGGAAGGCAGTAGGACTTGACCCCTAGGTTGACCAG
GGCTTATTTCTGCTCTGCCAAGCACAGGACCTGGCTGGCTTGTGGCATGGAGCTGACAACCCTAAACTGTGAAGGGCCTTTGGTGTCTCTAAGGCAA
TGGGACCCTCCTGAGCAGACTGCCGCAGCCGTGCTGGATACTGGGATGTCTCCACCATGTCCATTCCCTTGGGCTTTGTTGCTCCTCCTCAGCCCAG
ATTGAAGAATTCTATGTATGTGTTAGAGCTTCTTCTATGCTCACCCAGCTCTTGCTATGCATATAAAAGTGTCAGGACCTAGGGACAGGTGCCTTTG
CATGCTGTGTGGCTGTCTTTAGAGACCTAGAAGATATTCTTCCAAGACTAACGGTGAGCAGCTGCTTTGCACAGACCACACGTGGTGGGAGAAGGGT
TGGAGCCCACATTCTGCCTCCACTCGGGGTCAGGAGAAGACTTCCTAGTTAGAAGGAGGAGAAGCTGCTTGTCTNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNTGTGTGTGTGTGTGTGTGTTGGGGAGAAGCTATGTATGCATTTGTGGGAAGGGGGTAGCTTATTCATGTGTGCATTTGAGCATG
GAGCCAGAATACAACCTATGGTTTCATATCTTGGGCACTCTGTTTAGCTTTGGAAACATAGTCTCTCACTGAGACATTAGGTTCCCTGATTCGTCTA
GGTTGGTTGGCTAGTGAGCCTCAAGGATTTCCCTGCTTCTGCCTCCCTAGCTTTGGGATTACAGGCACACCACCACACCCAACCTTTTCCCATGGGT
TCTGAGGAATGAGCTCAGCAATTAGATAAGCCATCTTCCCTCCCTAGCCTCAGAATTCATGCCCATAGCCTAGCCCTGAGTTGGTGTTTCTGGGCTG
TTGAGTCTTTCCTTGACTTTCACAGCTAAGACATTCAGCACTATTTCCTGCTGAGAAGCCAGAATGCTCTCTCCTTTCTTATAAAGCATATTCCTTT
ATGCATCAGCCATTTAGGAACCCCAGAGCTAGACTTGATACTCGATGTTTGCACCTGTGCTGACCTTGGGTGACACATTCGTCTTCTCCTTTGCCTG
ATCCAGTGTTTTCATCTCCATTCTGCCATTTATCCAGAAACATTTTGTATTCTAAACTCCTTGCGGGCAGACCCCATGCCTGGAGCATCTAGAGTTC
ACCACGCTTGGTCTCTGTGTAGCGGCTACTGATGCTCCATAAATATGTTTTGGCCGTCAGACAGGACGTTGGCTGCCAGCTGTGCTGAGTTCTTTCT
GAAAAGAGGCAAGGCAGAGGAAAATGAGAACGAAAAATTCATTTTGTCAGCATGGCAGCAGTATGCCTGTGATCCCTGCACTCCAGAGACTGAGGCA
GGAGGATTGCAAGTTTGAAACCAGCTTGGGCTCTACAATGAGAGTCTGTCGACACAAAAGAAAGAAAGAGGGAAAGGGGTGGGTAGGGGAGAAAAAA
ATGGCTGTTTCTGAGAAGCGGCTCTGAGCTGACAGTTTTGAGTTGGGGGAAGCGCATCCTGTATCTTCCCCAGAAACCTTATCTCTAGGGATGCTGT
TTGCAGGACTGACGGTGCTTTAGTCTAAGCTTGAAGGTGTCATCCCACCTCTTGGGAATCTCAAACATTGTAGCCAAGGATGACCTTGACCTTTTCC
TCCTCTTTCACTGCCCCAGTGCTGGGCTTAGCCCGCACCACTGTGCCTGGGTTTATTTGGTGTTAGGGATTGAACCCAGGGCTTCATGCATGCTAGG
CAAGCACTCTACCAACGGAGCTATGCCTCAGGTTGGGGACTTTGTGTCTCCCATGGCTGTGCCTTCTCTTTGTTTGCTCCCCCCACCCCATGCCTGT
CTGTGGTGTTCTCCTTGGGAGATGACTAATTGATGCTCTTGATACCACTTCCTAGGTGAGACACAAGTACCCAAGTGCCCCCTTTCCATCTCCCCAG
CAGCTATTGCAGAACTTCCACCCTGTAAAAATTCAGGGCGGCATGCGGATGAGAGAGAGAGAACACAGGAGACCACACTCAGGACCACATAGGGTGG
CTGGCCTCTCTCAGGCCAGACGTTCCCATTGGCAAAGCTGTGGGGAAGACCCCAAGTAGACCTGGACTAAGCCACATCACTGCTGTGGCAGGAACCT
CACAAAAAGGAGAGGAGAACACTCAATCGTGeATTGTGGTGCCTTTGACCCCAACACTTAGGAGGCCAAGACAGGTAGATTTCTATACCTTCAAGAT
CAGCCTGTCCTATGCGGGAAGTTTCGGGACAGTCAGGGTGATGAGAGACCCTGTCTCAATAAACAAAAATAGAAGTAGAGAGGAGCTTCAGGGTAGG
GGAGCACAGCCCGCCAACCACCTGGCAAGGCTTCAGTTCACCTCTATGCTCCTGCCCTTAGTTGGCCCTAGACCCATCCCAGTCCCTTGACTTCAGC
CAAAAGAGCATCCAAATCTACTGTAGGTGGACCTCCATAAGGTCCAGAACTTTCCAAGGATGACCATGACTCTGGCATGCCTTTGAAGCTAAAGCTG
CACCCTGTCTCCAGAAAGTGGGCAGGCAGGGAGCAGATTTTAATCATCAGCGAGGAATCCATCTGTCCTTTTTGAGGTTCCTGCCTCAGCCTGGTGT
GCACAGAGGGAACACATCACCTCTGTCAGCCTTAGAAGCATGCTCTTGTTCTCCCCATTCTACAGGTGACAAACTGAGCCCTTGAGAGAACCACTTC
AGGGCCTAAGGCTCCTCAGGCTGTGGACTGAAACTCCAATTAGATGCTGACTGTAGGAAGCCAGGCCCTCCAGGGTTCACAGCCCCTCACTGATAGC
CAGGAAGCTCCCTCAAGGTCTATCTAGACTTAGTCCAAGCCCAGACAATTTCATTTGGTCTCTGGGATGTGAACATCACTGAAGGGTCTCAATCTGC
CCTTGTGATTGGATATGTTGTTCCTGGAGTCCAGGGCAGTCCTTTGGAGGTAACCTGAAAGTGCCATTAGCCCAAGTTATGAGGGGGGTCCTGCCCA
GGTCTTTGGGGCAGAGGGCTCTGCACCACTGTCTGACAATTGTGCCCCTTTCTCTGGGGACAGGAATCTGAAATCCCAGGGCCAACACCCCTAGGGC
CCACACCCACCCTGGTGATGCTCCAGGCATCCCAATATCTGGGGAATCCTTCCCAGATGACTGTAGGCCTCCTGAGCTCTCTGGGTTTCTACCTAAA
GTCTGTTCTATTAGAGAAGATGACAGTACTGATGACCCAAAAAGAGGTCAGGGCAGGGTATTTGCAGGGGCTGTGGGTACCTGGAGGGTTAGCTAGG
TGTCCAGCATGGTGAGCCCTGTAGAGGCTGAGGGCTATTTCTGCCCATGTCACCATTTTCTAGAATGAACTCCTTGGAAAGACTGATTTTGCTTACT
ATTTAAATTTTGTATTAGTAACACACACACATATACACACTTTCTGGAAGCACATGGCAAGACTAAATCAAGAAAGCACATGTGTCCTGTGGAAGTC
CAGGACAAGGATCTCAGTTTGAATGAAGAGTATTGACATTTGTTAACCTGAGGTGAGGCAGTTACGTCTCTGGTACCTGACAAACACTCAGCCTTCA
TTTAATAATCTCGTCCCACACACTCTAGCCAGGATGAAGGAGTTGGATGGGGAAATGGGTCTGTCTAGGTTCTCCCAGCACAGAAATCTCGGAGAAG
GCTTGACCATCTTGGTGTCCCCCGCCCCCAGCCCCCTCCAGCCTTGCAACATGGGCTCACCCTAGCAGGAGAGGAGGCCCTTGTTTAGCAGCAGTAC
CAGGGACTGTCCCAGCTGTGTTCCTCTCACCTTCAGGGCCCTCTTTGCTTCAGGGCCTCATCTTGCCACTCTCGGTCTCAGAGGACCTTCCCTGTGC
TTGTGGCTTTAGATGATGTGACCAGAAGGGCAGCGACTGCATCAAGGAACACACCCACCTGACTGTAGTGTTCTTTAACCCCAAGGCCGACCTTGAC
CCTGCACCACCCTGCACCTTCCAGCCCCAGCCTGGCTCTCCTCCTGGCTACTCTCATCTCTAGGATGACAGTCCCGGGCTTCCATCTATGTAGCTGG
GTGACCTTACTTTCTCTGAGCCTCAACTTTTCCCTCTGTAAAATGGGGATGGCAAGGATTAAAGGATGTAACATTATATGAGACAGACAAGGTCTGG
CGTCTACACAGTGAGTAATGCTCCCATTTGCCATCATTAATCTCGCTGTCTATTTTGTTGTTTTCGCCATGCATCACCGCTCTGCGCTTTTGTGCTC
GCCCCTGCCTTCTTCTGGGGAGGTTCCGAGGACTCCTGTCCGCTCTGTGAAGTGAGGTTGGTACCATTGTCTCACCAGCACCTGCTCCACCTGAGCT
CCTCCCGGGGTCTCAGTGGGACTCCTTCAACTCTACCCACCTTGCCAGGCAGCACGGGACACCACGCAGCATCTGGGGACAGGTCTTAAGCTGAGGG
GCACTGCTGTGTGTGCCCTGCTTGGCAGAGGCCCAGTCTTCCTGCTCCAGGTGTCTTCCTCCTCCCAATGTCCTCCTCCTCCTCCTCCTCAGGAAGA
CTGACTGACACTTGTCATCTTTCTTGACTTCTCAGTCACATCTCATTGGTATCTATCTTATCCTTCCAAAGAGGGGACCAGCTCTTGAGGACATGAA
TTCTGCTCTGTGTCTTGGTGACCCAACTGAGCTTCATATAGCAACTCTGGGGTGCAGAGCACAGCTTGGGGAGCACTGTCGTTCTAGAGATTGTTGA
GCCGGTGACTCAGGACCCCTCCTCAGAAGCTCCGGTGCGGCTTTCGCTGCTTCCCGTGAACTGGATAGCAGCTCCCCCTCACCCATGATCTCAGCAG
GGCCCATCCTTGAGCTTCTAGCCTTTCGTGGACACATAGCTCTCTCGCTGCATTTGAAGAAGAGCTTTCTGTGGAAATTGGCACGTCTCAGACTTGT
TGGCTGGTGGCTCTCAGACTTTCAGAGCCTCCATTTCTTCTATGTAAAACGGCGGCGGGGGTGCCGTGTTGTCAGGAAAAGTTGTGAGGTCCTGCTA
CATAGGGGCTGGGCTGACAGGGACTGATCATGGGTGGTATGCAGGTCATTCCAGTCCAGCGAGGGTTGTCCAGGGACTCAGTACCGGGAAAGAATGC
AGGTCATCCTGCCCTAGAGAAAGGGCCACGCTTGGGGAAAGCTATACGCCTTGTGATGGTGTGCCCCAATTATCACCTTATTTGAAAACCAAAATGG
GAGTGGCTCAGAGCAGGATGCAAGTTATTCAGTGAGGACCCTGGTCTGACCCTGTGTCCTGGTGTCCTGCTCAGCTCCTGGGAAGAGGTAGGCAAGT
CAGATGTGGTGGCTTCAGGGAGTTTTACTGGGGCTCCTGGGCTGGCCTGTGCAGGTTTAATATGGCTGAGGCATCGGTCATTTCATCCCTGAGAAGG
GTGGTGAGTAAGCCTTGCCTGGGAGGCTGGGGCCGCTGTACACATCTGTCTTTAAGGGAAGAGGGCCCTGCTGTGCTGCTTAAAGTGTCACAGACAA
GAATGGGTGGCCTGTGTGTGACTGATATGCATCCTACACAGACACCAGCTGTTTCTGCTGAGAATTGGAGAATGAATATGCTTTTATTATATAGTAC
TTTATGCACACAAAGCAAACACATCATCTATGGGATGTCATAACAAGATGAAACCTTGTGCTTCCCCACACCCGGAAGAACCAGAACATCAGCATCT
GTCCTTAGGCTTCTCCCCTCCCCACCCCATCACCATCTCCTTCCACATGTGACCACATCCTGATTTTCGCCATTTTATAAAGAGAGATGGTGGAGGG
GTGGGGGGAATGTCTCTCACCCAGTCTGGAACTCACTAATTAGGCTAGACTATCCAGAAAGTCCCTAAAGATTCTCCTGTTGACCCTTCCCCAGTGC
TGGAGTGTTACCACCACACTTGTTTGCATGGCTGCTGGAAATGGAACTCAGGTCTTAGCTTACAAAGCTTACTGATGGAGCCACCTCCTCAGGCCCT
GCACAGGGATTCCATGACTGAGATAAAAAGCCGGCTGGATGTATGGGCCCACACCTTTAATCCCAGCACTGCAGAGGCAGAGGCAGGTGGACCTCTG
AGTTGGAGTTCCAGGACAGCCAGGGTTACACAGAGAAACACTGTCTCAAACAAACAAACAAACAAACAAACAAACAATAACAAAAGATAAAATGTAA
CCTGAGATAAGAATGCCCACAATACATTTTTCACTTTGCTTGTTAAAAAAAAAAAAGAGAGAGAGATAGTTTGTGTGTGGTCTTCTGTGGTTTCCTC
TTCTTTGTCTTATTTTCGAGATGGGGGGGGGGGGTCTTATGATATAGACTTAGCTGGTCTGGAATTTTAGTATGTAGACCAGGCTGGCCTCGAGCTC
ACAGAGGTCTCTGCCTGCCAAGAGCTGGGATTAAAGGTGTGTGCCACCACATCTGTCTTTGTTCTTTCTATTTTTTGTTTCTAAGTTCCTCATTGTT
CCTGAGTCTTTCTATATTTCCTTCATTTTCCCCACTGTAGAGTAGTCACTGTGTCTGCTTCCTTCTTTTTGTGTACACAGGAGTCTCTCTGTGCCCA
TGGAGGAAAGCTCTCCCTCCATACACTTGCCTTGGAGTGGAGCAGTAGGTCCTGGCTGTTGAATGTTCTTAGAGTGATGCCTAGCCATTCCTAAACA
TGCTCCTATCTCCTTCCTCTCTTTCCTCTCTTTCCTCTCTTCCCTCTCTTTGCCCATGATCTTCCAAAGCTTTTTCAAACTCAGAGCCCCCGTTGTC
CCAGGTCTCTTACCAGCTTGGCTTCTGCCTCGCCCCCACACTGCACATCCTCCAGCTCTTCGGAGCATCATTTGGATGGAGCACTAGGTCTCAAAGT
GTGTCTCATCACACTGGTCTTATGTGATCCTGCTGGGAAAGGCTTCTAGGGACAAGTAAATTTAGAAGTTAGCCTTGCTTGTTGGAATCTCAATGCC
ACCAGGTACACCACAGAGGTGACCCCTTGCTTTCATTTGTCTAGTCTTTCCCCAAAATAGCTAGCCCAGGAAATCCTCCTGCCCCTTCAAAATTCAG
TTTGGTTTTTATGGTCTTTATTTTCCCAACTGGGAGGCTGCGATTTTTTGCAATGTATATTTTTTTTAACATTCTGCATGAATTGAGGAAGTTTAGT
CTAGTATCTCCATCGCTTGGGGGTCTCGAGATGATGAATGAAAAAGCATAAGTGGTGAATGAAGGCAGCAGTTAGAGAAGCAGTTGGCTCGTTGCTC
TGTGGTCCAGCTGGGTCTCTTGTCCTGAGGAGTCAGAAGCTATGGACAACTTGAGGTTGTTTGGCCCGTGTCCAACCCTGCCTACAGTGTTTCTACC
CAGCCTTCCTCCAACACCCACGTGGAATGAGTCACTCCCATTAAGCCTCCTGTCACTGGTTTGGGGGCATCTGTTTCATAGGGAGGCCTGAAACCCT
TGCAAGGGCAGAGGCTCTGTTCCATTTGCCCCCTAGGTTCTCAAGAGTGGCTGCTTACCCTCTGGCTGGTGGATGAGTGCAGTCGGAGGAGGTCTCC
TGTGTATCCTTGCCAGGGACTAGAGGGTACTATGACAGTCTGCAGGCTCTGGAGTCAGACCAGATGAGCCCTCATAGACCTGCCAGTCTCCACGCCT
CAGCTTGGGAACTGGGCATCTTCTCTTCTTTCCACCCACAATTACTCTACTGACTCTGGGAACAGGGTCTCACTTTGTAGCTCTGGCTGGCCTGAAA
CTCATTATGTAGACCAGGCTAGCCCCAACAATTATGACAACCTCCCGCCTCTGCCTCCCAAGTGTAGTCATGTTCCACTGCCCTGGCTCTTTGTTTG
GTGGAGTTCTGTTTGTTTAAATTGAGACAAGGTCTCATGGAGCGTGGGCTAGCCTGGGACTCACTATACCGTGGAGGTAAACTCTGATCTTTCCTAC
CTCTCTCTACAAGTTCTGGGATTAAGGGAGCACACTTAGTTAATGTGATGGTAGAGACAGAACTCAGGGTTTTGTGTGCATGAGGCATGAATTGAGC
TACAGCCCCAGACTGTTTGTCTGTTTTGAGACAGGGTCTCATATGTAGCTCTGGTTGCTTGCCAAGTAAATAAGACTTGCCTTGAACTTGTGGTGAC
TCCCCTGCTGCAGCATCCCAGATACTGGGATGTGCAGGTGTGCACCACCATGTGTGACATTGCTGGGTTTTATTTTGATCCTTTGTTCATCCCTACA
AACTAGAACCCATAACAGTGCTTGGTTGTTTTGGGGTTGGGAATGGAGGGAGCGAACCTACAGGTAGGAACTTGGGAAGCCCAAGGGTAGCCAGGAC
TTTCATGGCTGACCCAGGAAGCTCAGTATCCTGAGACCAAGATCTCTTTGACACAGCGTGGCTAGAGCGTCCTGTGCTGTAGCAGTGTCCTAGAATG
GAGGGCTCACAGGGCGTGGCTTACTGCCGTGGTCCTCAGCCTCCTGCACAGGATGGTGTGCGCGTGGGATGCTTGCACAGGAACAATTTGAAATCAC
TTTTCCCTGGGCTTAGAGCGCCAGGAGGTCAGATAGCAGGGCCTGAGGATGCTAGAGGCCCACTTACCATTCTCAGGCATACACCACTTGGCCTTGA
AGTCGGGGGAAGCTCCTCAGACCTCATTAACCTCCTTTGTAAGGAGCCTCACCAGCTCCAGAGCACCTGCTCTCTTCTCTTCGCACTGGTCAGCCTT
AGTTCTAGATCTGTCTTCCCTGGCCTCCCAACAGAAAGAGATAGATCGTGGGGCACTGCAGACAGGCTGAGGTGAACGTCTGATGTGACACAGGATG
CCTGGGGACGTTCACTGGGGAGCAGAGAAACCTGGGCATTTATTTCATCCCAGGTAACGAGCTGTAGTCCCCTCCACAAGGGAGAACAGAGCAGGGG
TGCTTCAGAGGGGTGGAGGCACGTAGGTGAGATTGCCAATTGGGTGTCCAAGAGGCAAAGATGTCATTGAAATGACAGAGCCAGAGAATCTAGTAGA
GGGAGACTGAGATCTTTGCGGCAGAGGAAGGAAGACATTCAAAGCATTCACAGACCACTGGTCTTCTGAGAGCTCGGGTAGTGTGAGAGGGGGAAGG
GGAGATGGAGGTGGTGGTAGGCTTTATCCTTTCTGCAGCGGTACAAGCTAGAACGCGTGGTAACAGGGTGGTATGAATTTCCCCAGAGGTGTGGAAG
GCTCCTGTTGGTCTGATTCCTCAGCCTCCTCGTGAGACACTCCATCAGGTTGGACCTGAAAGTACTCAGATCCCTGCCAACCGCCATCTTTTCAGAC
TCACAGACAGTCTCCACATTGCTGCCTGAGGGATCTGAACTCAGTAGCTGACTGTCACCCCTGTCTCTGTCCACCTTGACCCTGTGTGTTGGTGTCC
TACTTACATCCCTATGTCCAAGGCTCCTTCCTTTTGAGAGCAGAGAAATGTACACAGAGCATCCCCTTTTACCTCCTTCCTTCTTGGTCCAGTCCCC
ACCACTGGGAATACCCTGGATATACCTCTTTTGCCCCAGTGTGTGTAATACATCAAGGGTTACACTCGGTTTGGAAGGAAAAAATGATTTTTAAAGC
AATAACAGCAACAACAATGCCAAGTGTGGTGGTATCTACCTGTCATCCCAGCATCTCGGAAACTAAGGCAGGGTAATTTCCATAAATTAGCATAGCC
TGGTCTGCATAGTGAGTTCCAGGCCAGCCAGAGCTACACAGTGAGAACCTATCTTAAGGTAATATAATTTAATATAATATACCACTCCCCTATGTGT
TTTGCAACCCATCCTGCCATGTGGCCCCTTTCTGTGGGTTTTCTCAGCTCTGCTTGGAAGTTGCCTGTGTCCTTGCTATTGTCCTTTAGTCCCTGTG
TGCATAGCCCTGTTATTAGATTATACAGTCCTCCGGGTGAGGGGCCATGACAGCGTTGAGTTAGTGTGGGACACATTTTGGGTGCAGAATAAGTGAA
TTCTGCCAGAATGAGTCATTAGCGGAGAGTGGATGGAGGCCGTTAGACACAATGGCAGCAGCATAGGAGGCGAGATGAAGCCATTCAGACCCCTGTA
CCTTCTGAGTCTGCTTCTCGTTGGTGCAGTGCATTAGCCTGAACCCCTTGGAGCTTTTGAAAGAATAAATCTCAATGAGCAGGTTCTGGAAAAACAG
CTTTGCAGTCTCAAAGGTGGCCAGCTCCCCTGAGAAGCCCACATCCACACTGGTTCCGAGAACCCTCACCTTCTGGGCTTGCTATCTTGGAAGAGGC
CCAGTCACCTAGAATTCTCTGTGGTTATTTGAACCCATGTGTCAACCCTTCTGATTCTGGGACAGAGCTCTTTTGTCCTTGAAGGATGCCTGGGACA
GAGTCCAGCACTCAGCCTGAACAACAAGACAGGCTGGGTGTCTCAGCTCTCCACTGACACACTGTAGAGTCTCCCACCCCAAGAAGTCCCTTCTTGC
TTTCTTAGCAGCCTCAGTATCTCCATCACCAGCTGTATGATCCCAAACATATTACCCGGCCTCTAGAACCTGTGTCATCACCTGTCAAGTGGAGATA
ATAATGTCGTTGGGTTCCTTGGCAAATTAAAGGATGTATTTCATGTAACGTGCTTTATATGATACCGGGCACAGAGTCAGGGCTCAATAAATGCTGG
CCTTCACAAATAATAACCATTGTGAGTGCCCTGGAGATGGTTTCACCCCCTCCACTCCCTTACACCAGAGAACAGGAAATCATTTCCTCACGGCCTT
CTTAACAACTGTCTCCCCAAATAAGTGCACCCTATATTATCTTATTCACCCTGAGTGTGCACCTGTAAAGCGGCCCCTTTATTCCATTTATAGCTGT
TTCCACAAAGAGGCCCCCATTACCTCATCTCCCTCTGTTTTGTGGGCTCCTCCTGTCTGTGGCCACTCAGGGCCAGTGTGGGTTCTGAGGGAGCCTT
GGCCACAGGTCTGTGTTGGCATCTGCCCTCACGCGCACACACACACCTTTGTAGAAAAGTCTATAATTTCTTCAAGCTTTGTGGCACATGCCTGTAT
ATCTGTCAATTCAAAAAGACTGAGACAAAAGGATAGAAAGTTTAAATGAACTATATGTCTTTAATCCCATCACTGAAGAGGCAGAGGCAGGTGGATT
TCTGTGAATTCAAGGCCAGCCTGGTCTATATTGTAAGTTTTAGATCAGCCCCGACTACATACATTCTCACCAAAGGGGGTAAGAAAATGAAAGCTCA
GAGTGGGTCGGAGAGCTTAGTAGGACTATCTATCTCTATCTCTATATCCATATCTATATAATTGGAAAAGGGCTGAGGAGGAATAGCCTTGCGTTCA
GTACTTGCCTAGCAGTGTGAAGTCTTGGGTTTAATCTCCAGTACTGAAAGAAAAAAAAAATCTATTGGAATTTCTTTATTTAAATGTGTGTGTGTGT
GTGTGTGTGTGTGTGTGTGTGTGTGTGTGTGTATGACAGAGAGAGAGAGAGAGAGAGAGAGAGAGAGAGAGAGAGAGAGAGAGAGACTTTTGTATAC
AAAGCTTGTTCTTCTCCAGAAGAAATACAACAAGACATCAGACTATAGAAGCAGAAATGCCTGAGGTATCAGCTGGGCCCTGCAGCCTCCTGGGGTG
ACAAGATTGGGCACAGTGAGAGGACATGGGTGAACAGGGAGGGCTCAGCCTGCAGAATGTATCCTTTCCCCGCTGGGTTATGCACAGGAACTCAGAA
AGATGTGGGTCCCTGTAAGAGGCACATCTGGAGACCTTGAACCCAAAACTTCTGTGGACAGCCAGCATGTCTGGAGCTCCTGATACTCACCAACATG
TAAACCCCACAGCAAGACTCAGCAATCTGGGTGTAGAAATTAGGAGAAGGGAAAACTCTCGGAGGAAGAGAGATGCAAGGAGGAATGTGGGCAAGGG
GGTGGGGTCCCCCAACAGTGCCAAAGACAAGGTTCTTTTGAAAGTTTTTTCATGGTGACCTTCCTTTTCGAATGGATGTTGAGTTTAGCAGCGGTTA
GCAGTGAGGCTCTTAAACTCCAAGCTTGGAGCCGCCTGTGATTTATCCTGGCCTATGTAGATGAGGGACATGCCCAAACACTTTTTCGCTCATACTG
CTGATCAAAAACAGTTTGGCTGCTGCGGCTGTCAAGATGGAGGCTTGAGAAACCTGGAGAGCTAGAATGAGGAAAACACCAGGCTTGGGAAGAAAGG
AGGCTGATTCTGGGTACCAGGATGTACGGCGTAGGGCGGAGCAGATAGGGCCCAGCGCTGGAGCCCCATGAGCGCGTCCTAGTCTCCTTTGCTCTTG
GCTTGCTCCTTGAAATGAAGCGAAGACCCACTGAGGCCCAGACTTCTCCATCCTATGGTGCAGACATCAGACTGTGAAATTCTGTGTGTAGCCAGCA
TTGAGGCTGAGCTGCCCTTCTCTGAGTGCTAGAATACCTAGCGGGAGTCCCGCTAGCAGCTAGGAGTGTGGAGTGGGCACTCACAAACCCAGAGAAG
AAAGGCTAGGCTTGGTGGAGAAACTGGAAGCAGGAGCCCAAGACTGGTGGATGTGACCGTGTGGGTGCGGGGGCAGGGGGTGCGAGGAAGGTGTTGA
GGCTCGCTCCCGGGAATGGGCCCGGGAGGGAGGAGCAGGCAGCGCTTTGAACTTGCGCTCAGCCTCCAGAAGAGAGGGCGGGGAAGTCCAGGTGCCG
GCCGGGCTGCTTTCACCTGCGGATCCTTGGCGCTTTGGGGGACGAGGCGCAAGGAAGACCTCCTGGGAATGCATGAAAATACCCCACCTTGAAAGGC
AAGGAGCGGAGAACATAGCCCTGAGCGCCGGGTGTGGCGCTGCTCCCGCCCCCATCCCTACCCGCCTCACGCTGCAGCGCGCACTGCTGCTGGTGCT
GGTACTGGTGCCAGTGCCCATTCCAGGGCCTCCCCGACCTGTTGGTGCTCCCAGCATCTGAGCCATCAAGGTTCCTGCCCACAGGTTTCATCTAGAA
GTCCCCACAAATGAGTTGGGAGTTTTCTATTGTTCTTCAAACTTTTATTATGGCAATCATTAATGAAGGCTGTGGATCTTGCTTGGAGATTTAGGGC
TCAGTTGGTGGAGTGCTTACCTCAGCATACCTGAGGCCATGGTTTTGATAAACCCCATCCTACTTGGATGGGGAGTCAGAAAGGTTATAGCTAGTCC
TGGGTCAGTCAGCTTATTCTACATAGCAAGTTCCAGGCCAGCCAGAGCTACATAACGAAAAGTAGAGGAAATAATATAAGGAGCAAAGAGTTTACAC
ACCTTATAGTGCCACCATTTAAAAATGTTTTAAACGTGCACGTTTGTGTCAAACAATTACAAAGAAAAATTCTATTCCGCATGAGCGCTGAGATTCG
ATATGTAAGGCTGCCCCCACCCCATCAAACCGCCATGCTGGTTAACAAGACGTCTAAGAATTCCCGATGATTCAGGTTGCCTAGGTGTCCTCCTCTC
TGCCTTTACCCAAAGGGTTTTCAACGCAGGCTCCACTCATTTCTTTTCCCTTGTCTTCATTTTAATCTCAAAGTAATTCATTCTCCTTATGCCCAAT
ACACTGGCTCTTCAGAGAGCTATGGTTTGCAGTCTTTGATCATGTGACCTGTTCTCAGATCATCTGCCTGCTTCTTTAGAGTCATATGACTTGTGCT
TCTGTCTCTCGTCTTCCTGACAACTGAGGGTTACTTAGGCGAACCGTCTTTGGCTGGTGAGCTTTATGGAGGAGGTTGGGTACCCCACATTGCGCCA
TCTCCAGCCATCCAGCTCTCCTGCTTCTTTGGAGGTGTTTAATAGGGTTTCTGAGTCTCTTTCCCAGATGGGCTGGTCCAGCGGGTTAGGACTGGAG
TCCATGTGTCTGGGTTTTGCAAATCTGCCCCTTTGCTGGCTGTTTAAGTGTCTCACCTGCCTTCCTGGCCTCAGCAGTCAGCTGGGCAGTGCTCATC
CTGGGCCCTCCAAAGGGCATGGGGAGGGAGGTGTGGGAAAAGGTGAGTTCTCATGGCTCTGGCTCAGCAGATCTGAATGTCTGGATCTGGGAGTTGG
TAGTTCTTATAGATTATAACCACGCCTATAAACATCTGTATGCTCTCAACATCTAGCACACAGCTGGCCCAGTGTAATTGTCCCTGAAATGAAGATC
TCCACCAGGCTGGTGGCCCTCAATGAGCCCCTAGAAAGATGGTTTCTGGTGTGAGCTTCCAGCCAATAAATAGCTCAGCTTTCTGCATTTGAAGACT
CAGAGAAAGACTTCCCCAGGGTGTGCAGCAGTTGTGAGAAAAGTCCAGAGAGCCAGCAGGGTGTCTTCTGCTCTCAGCTTGTCATGGCAGTAGCCTG
AGTGGGGAGGAAAGACTGGGGAGGTCAGTGGGAGCCTCTGGGTCCAGTCCAAGGGGGTCCTGGGCCATTAGAAAAAGCAGACTCTCCACTTAAGTAG
TCACCGGTGAGGGAGGGAAGTTGTCCTCTGAAAACCTCAACTTTTAGTGGAGAGAACGGCTCTGTAGACAGCCTCAGTAGAGAATGTTCAAGATCTA
TCTTCTTCTGACAAGCTGAAGAGATGACCAGGAGGAACAGTTTCAATCCAGAGGGGATTTTTCTAACCTCAGATCTTGAGGGGTGACTAGGAGCTCA
CCGTGAGATGAATGGCCAGGGAAGCTGTTTTGTCTTTCACCAGAACTTGGCTTGAAGGGAAAGCTCTATGAGAACTGGGAATGGTTCTGACCTAGGA
ACCAGCTGTCTGCTTTCCCAGAACCATTAGCCAGGCCCTGCCAAGCCCTCACTTCACTGCACGTGGGAGACTGCCCTGCCGGAGAGGGTCAGCAGGG
AGGAAGTGGTACGCCGCTCAAAGGAACCAATGAAGCACACTGGGCTTAAGGAACGTTTATAGTTCTGATTGGCAGGTCAGTAGGCAAAGAAATAAAT
GGCAGAGCCCGGAGAGAAAAGGATGTGACCTAATCAAGCTGAGCTTGAATTTCAAGCCGAAAGCACAGGCCAGCCAGCTCTCAGCCTCTCTCCCCTA
GGCACTCCTGGCTTTTAGAATTCCTACCCCAAACGGGACTCCTCACACTTGGTCAACTACAGATTGCCTCTTCTGCGTAGAATGGGAGGCCCCATCC
AATGTATACAGAGCAGGAGCTGTGCTGGTAACAGAAGGCGTTCCAGTGTGCTGACGGAGGCTCGGTGAGGCACAGAGGAGTTCTGTGAGGTACGAGG
AGATGGGGCTCTGGCTCGAGGTTCAAAGGCCTGGACCATCCTCCTTTCACCTTGAACCAGTGAAGGACATTGGATGAGTTACATCACTTAAGGGGTG
TGTGTGTGTGTGTGTGTGTGTGTGTTGAGAAAAAGAAGTTAATTGTCCCACCAACCTTTGGCTTTGACATTTTATGACTTGGATCTTTCATGAGTAG
CCTCTCAGGCAGCTGGCTATGGGACCATTTACAGGAGGCTTCGAGTGGCTTTCCAGGGCCTCTTACATTCTGTCTCTCCTCCAGCAAGACATAGGCC
AGCCAGGGACATTTTCAGGCAGAAACTTCTGCTCAGCAAGCCTTTATTATTGATAACCTTCACGCAGAAACCCCCGTTCTAACTAAACCCATCGCTT
CAGGGAGGGTGGACATGCCCCCACTAGAAAAGACTAAATGCAGTCTAGCACAGTCATTTGCAACTGCAAGCAACCCAAGCAGGCCTTTGATCCTTGA
TCATCTTCCCTGTGAAACAGAGCCTGCCTAACTGTGGCCTCTGGGGCCACAAAGGCAAGGATCAGACCTGCTCAGTGGGGGTGGGGAAGGGAAGAAC
ATGGGGACTCACTTGGGAACTGCTTGGTTAGAGGAAATGACTCAGTCCTTGCCCTAACCTCCTGGAAGCCCTGTCTCTGAGACAGCTGGCACTTCTC
AGAGAGACCTTCTTGGGGTCTGAGGTGAGAGGCCCCTGACCTGGACCTGCTATCTGGATGTTCTGGGCTGAGTTTGCTTCTGGTAAGTCTTTCTAGA
GAGGTAGACTAGGCCAGGCTGCAGAAGCCCCTGTCTCCTTCTTTCTTGCCTTCCTCTCTTTCCCCTTTCCAATTCTCTGGATCTCTCCCTCTCTTCG
TACAGTTAACTAATTCCCTTACATCCTCTTTAGGACCCATCAGCCTGCTTCATGCTCCAACCCTAATTTTAGCCAGGAGGGACAGTCCTTCCTCCGT
TCTCCACTGTGATCAACTCCCCCTTCCTATGAAATGGGGTGGGGGTGGTGGCTCATGCTCAGGACACCTCAAAAAACAGGTTTCAACATTTCACAGG
TTTCAACATTTTTCAGGCGGACTTGTCACACATCTTATCAGATCAGAGCCTTGCAAGAAGCCCTCTTCGGCACAGAGTAGATGCCGGTACATTTCCA
GTCACGCTGGGCTCCCTTTGGTGGAAAGGGTTCACCCAGCTTCTTAGATTATCAGGATTGATACCACTTCCCCTCTGATAGGGGTCACCCCCTTGAG
TGACCCCCGACTGCATGTTTCTTTCCATATCATCTTCTTCGTCCCATAGATGCCCAGTTCCCGTCTCTTTGGTTCCCCAGCAAGGTGCACTGGCTTC
CTATAAAGGTTCCACTGGGCCCTGAAAGGAGCTTCTAGAAGTATCGGGTGAAGATGAGTCTCTGGGGTTACTTCTGAGTGTAGACAGTCTTTTTGGC
CAGCAGAGCCAGTAAAGTTCTGTTGTTTGAGCTCCACCGGGACCTCAGGAAGAGAGGAAGAAGGTCACCCTGCCCTGCTCAATGGCTGCTTCCTTCC
AGAAGTAATGGCTTTGACATTGGATTTCCCTCTTCACGCCTGAGAGTTTTGCCCCATGATGTGGGTTGCCAGCCTGGTATCACCCTTCCTTCATGTT
CTCCAAAAGACTCTGCTTCCCTGCTCCTGCCATGATAATGATAGCCTTGGCGACGTGGCGAAGACAGCCAAAGCCCAGAGGCTCAGAGTTCAGTCTG
AGAAGCACACCCGGAGGCTGGTCCCCAGGCCACAGAGAGCGGCTGTAGCCACTCTACTGGGTGGGAGGCCACCTGGGGGGGCAAACTCCACTCTGTT
TGTCAGAAAGGAGTCTCCACGGGAGGACCTGGCTGTGATGGCAAGAGCTCTCACCTAGGGAGGAAACAGACAAGGAACAGAGCAGAGGGAGAGACAT
TGTGATGGCTGACCGCACGTGCAGGCTGTTTGCCTCTGTAAGTGCTGATGAAACGGGAGCCAGGAACACAGGCTTCAGCCCCTAGCCGTGTGAGGTT
GGGCACACCATGCTGTTCCGGCGTCACTCCTCCCTGCCTGGTATATGATATCAACAGTCCCAACCTGAGATTTCCAGCTAGCTCAGCTTTTCCCTGG
ACCTTTTGGAATCTTCAGGTCTAGAGTGCCTTTCCACTGTACCTCCTGAGCAGCTATCCTCAAGAGGAAATGCCTGCCTTCTGGGTTCCTCTTTGGG
CTCTGCACTTGGCCATGGTCTTCAGCTGTCACTGTCCCCAAGGGTTCCTTTTCCCATTGTTTCTAGCCTCTTACAGATGCCATTCTCTTGGTCACCC
TCGTTCAGACCTTCTACATCCCTTCTCTTTGTCCTTATCCACAGCCTTCTCTCTGTTCCCTGAAGCCTTCATCTTGCAATGGCCAGTGGGCCCATCC
ACCGCCATAGCCTGTTAGCCTCTCTGGCTCACCTGAGGCCAGGCGACACCAAGGCCTCTGGTGTATCGATGTGTTGCCAGGTCCTCGAATCTCCTTA
GTGCCAACTGTCACACCAGCCTTCCATCTGTTTCCTAGCTTTTGATCTCTAGTGCTGCCCTTGTCCCATTGTAGGTGGGAGATAGATCAACAAACCA
AAGTCCAGCTCTGGATGCTTCGAGCTCTGCCAGCTTGCCCAGAGTGGTATAAAACCTGGCAGGTGGGGCCCCTGCCAGCCCCCCCTCCTCCTGAATA
GTCCAAGACGTCAGGTGAGCGATAAATGGTAACATGGGTACACCAGCTGTTGTCCCCAGCCTCCTAGTTCGAGGCTGCTCTCTGTTCCAGGTGCTTA
AGTGAGCCCAGCTTTAATCACCAGGTGTGTAGCTGCTCCTGATCACAGCAGTGGGGACTTACTGGCCCCACCCTGGGAGACTCAGTGTTCTTCCCTA
TGCTATGGTAACGATGACTGAGGACATAAGTGACAAAGTAGCCAAAGATCATCTCCTGCTCACGTGTGGCATCCACAGTACCCTGATCTGTAGGTGC
TTGAAGAGGGCTCGTTGGCCTGCCGTTGACAGATGTCTCCTGGCCTGGGTGCTCCTCCGTGGGGCTTCATTCATCTCTGTGTTCCTAGCACCTGGAG
CTCTGCCAGGTTTGGAGTCATCTCTCAGTAAGCGATGTTGAGTGAGCAGTCGCTGAGTGACTGTGTTTACAAGGAAGGACTTGAGCCCCAGCTGCCG
AGACCAAACCCTCCAGTTGCTTATCCTCGCAGGCCCAGACTCAGGCAAACTGGAAACTCAGCCCTGCCCTGGGCTCCACCCTTTTCCCTTCAGCCCC
TGTAATGTGAGCTAAAGTTATCTCTGTTCCCGATGGATCCAGAAAGGCAGATAGATATAAGGGATGGAGAGAGGTCCGAGGACTTCCCATGACTGTG
AAGGGCAGCATCTAGGTTCCCTTTGTGAACTGACAGCCATGGTGTCACTCGGAAACAGCCCCTTCTTGGTCATACGGTGCTGGGAAAGGTGTAGTTA
TGTGCAGAGGGCCCTGTGTGTTCCAGATCAGCATGGATTCTTAGGAAAGGTGGCGGCAGATGCATCATGGGATTTCTCCCTCCTTGCTCTTTATTGA
AGCTTCCTGCCATGGTGCCAGGGCTCACACAGGGCCCGTAGAGACTCTAGAAGCTGAGAGAACCCTGGCCCACCAGACTCAGCTCCTTCCCAGTCCT
GAGCTTTGTCCTCTCCCTTGTAATAAATCAGCAGCTCTAATTTCCCTAAGTGGAACGGCGATACTTGGGTTCTTTTTGTCCATTTCTTTGATTTTCT
TCTGGCTTCTAGAAGTTTCCATATTGCCCAGGAGGTAGATTCTACTGGACCAAAGTGGGTTGGGGCTTCTGTCTAGCATTGCCTAGAAAGTCAGCAG
ACAGAATACCTGGTTCTGTGCCCTTATGGGGTTTTGGGAAACCCTCTACCTTCGTCTCCCTCCCAATGCCTGGGCATTTTATTTCATGAGAGCCTAG
GCTTTTGGATCAGAATCTTGTGGTTATCTCTGAGATCAGGTGGGTGGCTACTTATCACTGTAAGAAAGAGAAGGGCAAAGAATCCGGTGCTCCCAAG
AAGAGTAAGACTCATCCAAACTCATCCCAAGACTAATAGATGAGCCAGAGACCTTCTCCCTGTGCTGTGGCCTGGAAATACACTCTGTCCACTCTGC
CAAGTCCCAGAGAAGCTTCTGAAAATGTGTCTTTATCACACCCTGGGATTCACAGGAAGGGACAGAGAAAGAGCTTGTTGGCATCGAGGCAGGGGTC
GTGGCCATGGCTGCTGACCTTTCCAGGCAACTGAGCTTCCCCGTGACCCTAGCTCAGACCZGTGCCTTTCTGAAAGTTCTCTGAAATTAAGGGATTC
AGAGTTGAAGAGATGGCTCAGCGGTTGGGGACTGACTGCTTTTAAAGAGGACCCAGGTTCAAATCCCAGTACCCACATGGCTCACAGCTGTTTATAA
CTCCAAGACCTGACACTCTCACGTAGACATAGATGCAGGCAAAACACCAATGCACATAAAATAAAGGTAAATAAATAAAACTTTTATAGAAAAAAAA
ATTGAGGGATTCAGGTAGACTCGGTCCTGGTCATTTCAGCTATTACTAATTGTATAAATCAGTGCATTAGGTAAGTTCCAATGTCAGGCAACTACCA
CTATTATTTCTAGAACATTCTCTTCATCCTAAACAGTCATTACTGTGGAGTATGAGCCTACGCCGAGACTGCTGGATCCTGTGGTATATAACAAATT
CTCTTTTTAAATGTTGTATATAGAGGAAAAGGAGGGAGTTTGTGCTTGCATCCGTTTCCAAACAAAAATAGTTCACCATTAAGGCAGCCTGAACTTG
GTGACACCCCCACCAGCATTCACCATCTCCCTAAGCTCTAAGAGCCTGTTGGGAAGCTCAAAACTTAAAAGCAAACAAAACAAGCTAGGCTGGGATA
CAGAGAAGGCTCCCTGGTAGGATGAGCCTGGTGTAAGCAGACAGAGCCAGCCTGATCTCAGCAGGGACTCAGTCATGCAGAGGGATGGCAGAGTCTG
TGGGTCTCTAAGGCTCCAGGACACCTCTTTGTAAGTCCCTGCAGCAGCTAAGCTGCCCTGAGTTCTCTGACTAGAGATGCTGGGGCTAGAGATGCTG
GTAGACTGCCTTCTCAGCAGGCACAAAGGCCTGAGTTTGTTCCCCAGTACTGATTTAAATACCATAAAATCGTGAGAAATGGACAAAAAGAACCCAA
GTATTGCCTTTCCACTTAGGGAAGTTGGTACTAAACTGGATGTGGTGGCCCGTGCCAACAATCCTGGCACTCAGGTAGAGCCAGGAGGCTCAGAAAT
TCAAGATCATCCTTGGCTACGTAGGGAGTTCAAAGCCAGCCTGGGATACATGAAATCCTGTTTCTAAAACAAAAACAAAACAAAAAAAGTCTGTATT
TCATGAAAAGTCTTATATATGTATATATACTTCTACATTTGAATGAAGTACTAACAAGCCACGTATGCATTCTTTCGGAACTGCTGCAGATCTTTTC
TCCATTTTCCTTCTAATTAATTACATCTGTAAGTAAAAAAATATGAATCAACCCATTGGAATGGTTATGTCGAGGTTGGATGAGCACATCTGCAGTC
CTGGTACTCAGGTGTCTGAGACAGGAGGATTGCAAAGCCAAGGCCCGCATGAACCACAGACTATGGCCATGTCTCAGAGCAACAATATCTTGGGCTG
TAGTAGCTCAGCAGTAGAGCCCTTGCCTTAGCATGGGTAAGGTCCCAGGCTCCCTCTGCAGCCAGGAAAACTGTCTGATCTTGTCACCATGCACACC
CACCCACAGCCCTTTGCAGAACAGGTGACAAAGAACTGGGTGTCAGTGGGAAGCTGACAGGGCCATGGGGATGTTTACAGTCTGGCCTAATTGGACA
GTGGCATTGGTCGCTATCATCAGCCTAAGCCTTCCTGACATCTATGGCTCATCCACCTGGCTGCTTGCGTCTGCCTGGGCCTGGATGGTAGGTGGTT
GGGATGAAGACGAGCTGTCCCTGGGGCTGAAGACAACAGAACCTTGGGTAGAGCAGGGCAAGTCGGGGACAGAAGCTGGGTATAAATAGTCCCTGCC
CAGCTGGGCAGGCCTGGCAAGCCCTCCGTCCTCCCCCTCACCAGCTGCCAGTGCCAGCCATGCCCCGACCTCCTTTGGGAGCCTGGTCACCATCTCA
GATCCTCACTGGAGGAAGTCCCTCAGCGGAACTGAGGAGTCTTGTCCTGTGTGTGGGCGAGACCTCCGGCTGCCCGAGTGGGGGCATCAGGCAGGCT
CTTCGGGGTGTCCTCTATTTTCTTAACTGGAAGGCCAAGTGTGCTGGTGTATGTGTGTGAAAAATCAATAGCTATTCCTCTTTGTGTGTGTGTGTGT
CATGCTGCAAACAAATGAGCCAGCCTTCTGTGGTGAGAAGGGGAGAAGAGGGTACGGGGACACTAGCAAAGGTTACTTCTAGCTTGGGAGCTCACAG
AAGTCTTTAGAAGTGATTCAGGGAAAGTTTGACTTGGGTATGGGCTGCAGAGAGGCACTCAAGGAGAGGGCTCAGCATGCGTGAAGGGTGTGAAGCT
GTTTCAGCTGGCCTCAAGGCAAATCTAATCAAAGCCTCTCACAGTGGGGAGATGGAGAAGATCAGGAGCCTCTCCTGTTGACTCTCCCAGGGGCACA
AGCTCTTCTGTAACGGGGACAAGAAGTGGGTTACACTGATATGTCTGCAGCAATGGAATGTCAGCCATGGTGTGTGGGCACGGCTCAGGGCCGGCAG
GGCATGTCAGGGGGGACAGCTGTCCTGCAGAAGTCAATCCCAAGGCCATAGCCTCAAGGCTGTCCATAGATCTGAGCCCTGTCCCCAGAGGCCCTAG
CTAATTACCACAGGATATCCAGATAGAGCTTGTTGAGACCTCTGACCGGAGCATCCTGGAAACCCTCCCATGCCCCTTGGTCAGTTTGAACATGCAT
CCAAGTCACACATGGCTTTGCTACGCTCAGCCTGTCAGTCACAGAGGGATTTGCTTGGCCAGCCCTGTCAGGACTGCTGGTGAAGAGACTCTGCCGT
GTCTTGGAGCCTTCCTCCTCGTGCTTGATGGGAAGTGGCTTTAGAAATTCTGATGTGCTGTATCAAGTCAGGGAACTGAGGCTCCGGGAAATGAGGT
TGGCGACCCGGCAGGGACCAAGTCCTGAGAGCATGGAACCTGAGGTGGCTGCCATTGCTTGAGGGATCCTGATGTGCATGGCACTGAGCTGTACATG
TTTACTCTTCCCGCAAGCTCTGTCTCACAGTGGAAGAAAGTGTGGTGTGGAGAAGTTAAGGAAATCGCTTGGATTGGCAGCTTGAGGCAGGCAGAGC
CCGATGTGAGCTGGGGTGAAGAGACAGGCTTGGAAGGAGGAGGGAACCTAACTCTGGGTGACAAGTGTCTACACTGAAGCCCTTGCTGGGGTTTCCT
TTCTAGTGTCAGTAGGGCTTAGGGATGAGTGGCAGGCCAGGGCTAAGTGCTGGCTTAGTGTGTGAGAATGAGCTTGATCAACAACACTGCAGAGAAA
GGGCCTGGTTGGGGGCAGGGGTCTGTAGGGAAGGAAGAGAAAAGACAAAGACATGCCAAGAGGGAGGCTCAACTCTGGCACTTTTACGACCCACAGG
TCCAGGCAGCTTACTTAGCTCCCTTGCCTCTGTTTCCCCGTCTGTGAAATGGGATGATATTGATGATGACACTCTCTTTACCTCATAGACTGGTTGC
GAGGATTAAATCAGTTAAAGACGTAAACATTTCAAACTGTGCCCAGCTCAGGTCAGATATACATCCATGTTAGGTGTTTTTGCGGGGCTCCCCTATG
CCTTTATGTGCTGTGTGGCTTCAGGCGCTAGGCTCACCATTGTTTCTCACTCAAACGTCAGCCCACCAGCGGGTAGGCAGGCCAGGCCTGAAGCCAC
CCAGCTCCCTAGGGACCCAGACAAGCATTGCCCACGAAGCAGAGGGAAGGCTTGCCTGGGAGCCAGGTAGTCTAGTTTCTAACAGCAGATAGAAACT
CTATAATCTGGGCAGCTAGTAAAGCCCCCACTGTGTGCTGGGTTGTTATCAAGAACTCAAGGAAAGTTAGTGAAGAGTTGGTAGTGAGCCGTGATGG
TGTGACACTAATGCTTCAGTGGTCACCTCATTCTTTTTCTCTCACTCTTGGCCAGTCCCAACAGGAGACAGTCTCTTGTAGCCCAGGCTAGCTTGCA
CCTCCTGACCCTCTTGCTTCTGGCTCCCAAGTGTTCAGGGATTACTACCCACCTTCCTAGCTTTCCCTTGTCAAAAGGGCCCAGGGCTCTGGTTGGG
GAGAGCTCAGTGGGAGATCAGTTGTCTGGCAAGCTTGAGGCTTTGAGAGGTGGGGTTGGGGACTCTAAGCTCAGGACAAGGTTCTTCAAGGCCACCA
ATTCAAACCCCCCAGTTCTGCCTACTGCACTCTCTCTTCCCAGTGGGGAAACCTGGACTTCATCCCCTGACAGCTGAGGCCAGAGGCTGCTGGAAGT
GGTGCTGCTGTGTGCCATGTTTTGCTGGAGCCTCTGAGCAGAGCAACCGAGGCTCTGGGTGTTGAGTGTGGGGGAAGGGTGTTGGGAGGCAGCTCAC
AGCATACTCTGATTAGACTCTGATGAGCCATATACCTGTTCCCAAGAAGCCCTAGGGCAAGCTAGTTGTTCTGGAAACCCAGATTCCTCATCAGGGG
ACAGAGATGATCAAAAGCTGATCAAGTCTGTTACATCCAAAGTTCTAAGCAATACAGAGTCCCTCAATGGCCGCGTTCGGTTCACCCCATGCCAACG
AAGCTTTTGTGTCTCTTCTGACCTACCCCAGAGTTTGCCCCTGGCCCCTCTTCCAAGGCTCCTGCATCCAGGACTTGACCTTAGCTAGTGCCTAGGT
CTCCTTTCTTCCAGGACACTCTGCCCTTTATTATGAGCCTGGAAGAGGGAAGGAAAAGGTAGAGTTCTGGTAGTGCCTTGCTTTTTCTACAGTAGTT
AACCCTTTCCCCCCCGAGTGTACTTACCGGCGCTGTAAAATCCCAAGGGTTGAAATGTAAGAGGTACAATAATGGGGTGGCTTGACTGAAGTGAGAA
GGTTGGGGGGATGCCTGCTGAGAGTTTCCTTTTGCTCTTGAGTTAAACTAGTCATCAAACGGAGCCTCTGTCGCTAGTGAGAGGGGAGGTGGCACTC
TAGAAACTTCTAGGGAACTATAAAGATGGTGGCGGAGGCCCTTGTAGACAGAGCACTTGATGCCATCAGAGACAAACAGGCTCTTGGTGTCCTCTGT
ACTCTTGGGGAAGAGGCTAAGAGGGCTTTGTGGGGAGAGAGAGAGAGAGGGGGGAGAGAGAGAGAGAGAGAGAGAGAGAGAGAGAGAGAGAGAGAGA
ACACTGTGCTAAGACTTATGCTCCCCCCCCCCCAAGATGGAGCTGTGATCACAATGGCCTCTGCTTCCTTTGTCCTTTATATTTGGATTCCTTCCCA
TGAATGGCTGTTAGTCTCCACATGACCCACGAATCCTGGAGTCCCTGGGAGATTGCTCTGCAGGAGCCCTGCCATCTCTACCAGGGCACCGGGGCCC
AGGCACTGGGCAGAACTTGATTCCTGCTCCAATTCTTCATTTCCAACCCAGGGTTATTTTTGTGGCACAGTACAGACCTACACAGTGCCCCCTCCCC
CAGAGGCTCTGCTGCTCCACATGCCCATGTCAGAGGGACACTGCTTGAACCGGTGCCGTGCCCTTTGTGGGACACCGTGTCCCCAAGGAAAGTCAGA
AAGCAGCACACAAGGTGCCAGGCTTCTCAGAGCCACGGATCATCCCTCCCCCACCCCAGCCCATCCTCTGGGAAGAAGTGACATCCATACTAGGCCA
CGTGTTCTGTGTCTACAGTGCCATCTCCATTGTCTGCCTGCAGGTCCCTGGCCCTGGGCCAGCAGTACACATCTCTGGCCTCCCAGCCTCTGCTCTG
CGGCTCCATCCCAGGCCTGGTCCCCAAGCAACTGCGCTTTCTGCCGCAATTACATCGAGATCATGCCCAGCGTAGCAGAAGGTGTGTGCTGGGCATC
CAGGAGTGCCAGCATCAGTTCCGGGGCCGCCGGTGGAACTGTACCACCATAGATGACAGCCTGGCCATCTTTGGGCCTGTCTTGGACAAAGGTACTT
GCCTGAGACTCGGTGGGAGGTGGCTGGGAGACTGGGGGTGGTGGGAAGGGTTCTGGGGACCCTGTCCCCTTCCTCCCACTGTGGGAAACCCTGAGTC
CAAGTTCTCCCTAGGAGGGTAATAAAGCTGGTGGAAAGTGGGTGGGGTCCATCTCTGGACAGGACAGGCTGTGTCCACATCGGCTACATCTTTTCTA
GTGCTACAGGGGGGTGGGTAGCCCTGAGGTGCCCCAGTGTCCTGAGATTGGAGGGCAGGCACCAAGGAGCCCGGAGCCTTCCTTCCCGGATGACACA
CCAGGCCTAGGTGTTGTGCCATGGAAAAAGTCTCTATGCTCTGTCAAAGCTGTCTGGTGCTGGTAGCACTACATGACCCCCGAGGGTCCCCTGAGAG
TTCCTGTTGGTTCCCATAGCCTGAAAGCAAGACGCGTCCATTAACCCTGATGTCCTGTGGATCACGCTCGGTGATCTGGAAGTCTGACCAGCTGTGA
CCCTAGTGGATGAAAGCCGGCTTTTGACTTTGCACATGGGGCATAGGAGAGGGTTGAATGGGGTAACTGCTCCTAAGAAGGGGACTTTGGCTCTAGA
ACCTCTCCTGTCCAGGAATTTAACGGCCTTAGCTGGGCTGGCCTCGACTGGAGTTTTGCCAGCCGATGGGGGCGAGCCTGCTCTGAGAAGGTTCGTA
AAAGCTGCATCAGAGCAAAAGGGAGGAGTCCGAGAGCTAGCGGGAGGGTGGGCGCAGAGTTTGGGAGAGGGGAGCTATCCCCCCCCTCCTTTGGAGA
AAGCCCTCTCCCCCTTTACCTGTGCTTCAAGTGCAAAGTGTCTGTACAACCTACAGGCAGAAAACGCAGGCTATTGTCGTGGGACAGGAGGGCAATT
AGCAAATCAATGGATGATGATTCTGTTTACGCTTCTAATACCAAAGCTAATTATTCCTTGTTTACATAAAAGGTTTGGCCTCTAATTGGGGCCAAGT
GGCGGCGGCGGCTGGCTTTTCAAAACTGATCTCGCCCGCCTGTTGGTCTCACCGCCTTCCGGCGCCCCACCTCAGTCCAGGTAGCGTTTAATCAAAC
TGCCAGGCGTCCCGACGCCTCCCCCAAAGCAACACTGGCCTTCTGATCACTCCTCCCACCCAGGATAGTGTCCATCGAGACCCTTTCAGTTGATACC
CGCTCGCAGTGGGTCTGATGTTTAGATTCGCAGCAGTGGGTTGTGCCACATGCCTCCGTCCCACAGCAAGAGGGCAGTTTGTGTCCCAGTGTGCAAG
GAAATATGGGGCTGAGACAGTGGGATTTGATGAAGGCAGTGGGGTGAGGTTCCAGGAAGATGTACCCGCGTCCCGGTACCCTGTGGGCAGGCGGTGG
GTCCTGGCCACTTTTGCGCACGTCCTAGCAGGTGCGGGGTGGGCGGCCGGAAGGCGCAAGGCCGCGGTCCTCCGCGGCTCCGGAGGGTTCGAGCGCC
GCCCCCCGCCGCGCGCCCGCTGCGGTCGCCGCGCAATTCACACGCCCTCTGGCCGCCGCCTCCCGGGGATCGCGCTGCCCGGCGCCGGGGCTGGGGG
CGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNCCCAGGCTAATCCCCGCCCCGCCCCCGCCACCCCACTGCCCCGGCGTGGGAACTGAGCG
TGGCGGGGCCGGGTCGCCGGAAGCCAGGGGCGCCCTCCCCTGGGGCGAGAGGGCCGGGAGGCCTGGATCACCGCCGTGCGCAGTCCCCCCCGGCTTG
GCTGGACTGCTGAGCCAGCTCTCGCTCCGCGCTCCCCGCGGGCTGCGGGGAAAAGTGGGACGCGCCCCTGCTGTGCAGGTGGCGCACGAGGGCTTCG
TGTCTGCAAAGGCTGCTGTGGCTACTGAGTATCTGGTTCCCTGCATATTCAGCACGGGCCACGACCTAGGAAAGGAGATCCGGCTCTGACCACCTGT
GCAGTATAGCACGACCCGCTCTGGGAACCTATAAAAAGAGATGGCCCGGGCCGTTGGCCTCGCCCGGCCCTGTCTGTAAAATAGGGGTTTAGTCATT
GTAGGGAATAAAGCACAGGAAAGACAGATCTGCGCGTATGCAGTACTCAGGGAGTGGCTCTCATTGTAACAAGGCTGGTGATGCCCGCTGAGACTCT
CATCTCTGAACCCTGGGCCTGAGGCCGAGAAACTGCCTTGTCTCGACCCTCGTGGCTTCGTAGGAGGAGGGCAAGAAATGGGCAGCCATGGCTGAAG
ATGCTGCACCTACTTTACAGAGTGTACTGTGAGGCACAGATGAGAAATCTGCCTACGGGGAACTGAGGTTCGAGTGGAACGAGTGGATTAGTAGGTT
TTAGGCTGCAGGTTCCAGTCTCCAGAATGGAGGACTCAGAACTGTTAGGAGGAGTGTTTCTGAATGTGCAAGGTTGGAGGCCAAGAGCCCAAGGACA
GGGCGTGGCCAACCTAACTCTTCAGAGAGCATTCCGGTCCCACCATAGACCCCTCCATCTTAGTAGCCCTGCAGCTCTGAACTCAGCTTGCCCATCC
ACAAAGAGAATACTAACTAGGGTCTGAACTCTTTAGAGCAACAGGAGGGCACCTGGCTCTCTTCCCCCAATGACTGGTGAGCCTTTGCGCTCTTCCC
CAGCCCTCAGGGCAAGGCTGTGTATGTACCCTAAGGCTCGAGGCTGAACATCTTCTCACACCXTCCCTCTGCAGCCACCCGTGAATCGGCCTTCGTG
CATGCCATCGCCTCGGCTGGTGTCGCCTTCGCAGTCACACGCTCCTGCGCTGAGGGAACCTCCACCATCTGCGGCTGTGACTCACATCATAAGGGGC
CACCTGGAGAAGGCTGGAAGTGGGGCGGCTGCAGCGAGGACGCCGACTTCGGGGTGCTGGTGTCCCGGGAATTTGCGGATGCGCGGGAGAACAGGCC
AGATGCCCGCTCAGCTATGAACAAGCACAACAATGAAGCAGGCCGAACGGTGAGACAGCGACCCACAACTCGGGGGCGGGAGGCACTCCCTGGACCC
CTGCCTTCACGGCCAGGTTCCCATCCACACCCCATAGTAACCCCCTGACCACCCTCCTTCCTCTCTTCCAGCTGTCCTTTGAATAGGTTTTTTAAAG
TATATTTACTCATTCTCAGTGTGTGTATTCCATGTTGTGTATGTGGAAGTCAGAAGGCAACCTGTAGTGGTCAGTTCTTCCCACCATGTAGGTTTCC
AAACTTGAATCATGCTTGATGCTGTTCCTGGATCTTTTGAGAAATCTGCCAAGGAGTGCAGTATGGCTGCCCCATAGGAACAACACCCATAGGCTAC
ACTGGTTGGCAGGCTGTGCCTGTGTACGTCTGTGATGTGGTTGTGGGTTGGGGACTGACTCTGAATCCAAAGTAGGGTTTCCATAGCATAGCCCTTC
CTATGCTCGTGGAACACTTAGCTCTCGGCCTCAGTTTTTCCATCTGTTCAATGGGAATAACAGTCCCTAGTTTGCACCACTTCTATGGATGAGGTGA
ATGTGAAGCCTGCTGGGGCCGGGGGTGTGACTAGGGACACTGGTTTCCACCCATTCTTCCTGGCACCAAGTAAGAGTAGAGGAGGGTGTGGTACAGA
GGGGAAGCTTTCTCCCGGTGTGTCAGTCTGAACTAGCTGCTGCCCCTCTTTCCCTAGACCATCCTGGACCACATGCACCTAAAGTGTAAATGCCACG
GGTTGTCCGGCAGCTGCGAGGTGAAGACCTGCTGGTGGGCCCAGCCCGACTTCCGTGCCATTGGCGACTTCCTCAAGGACAAGTACGACAGTGCCTC
CGAGATGGTGGTGGAGAAACACCGTGAGTCCCGAGGCTGGGTGGAGACCCTGCGGGCTAAGTACGCGCTCTTCAAGCCACCCACCGAGAGGGACCTG
GTCTACTACGAGAACTCCCCCAACTTTTGTGAGCCCAACCCAGAGACGGGCTCCTTTGGTACCAGGGACCGGACTTGCAATGTCACCTCCCACGGCA
TCGATGGCTGCGATCTGCTGTGCTGTGGCCGGGGCCACAACACGAGGACGGAGAAACGGAAGGAGAAATGCCATTGCGTCTTCCACTGGTGCTGCTA
TGTCAGCTGCCAAGAGTGTATTCGCATCTACGATGTGCACACCTGCAAGTAGTGAGCCAGGTAGGGCGCGGGGCGGGACGTGGGACTGCCCTCTCCT
CCCATCCGCTAGGCCTGCTCTTCTGATTCTCGGCTTCCCAAGACGAACAAGTTCCCAAAGCTGAAGTCAGAAACTAGGCTTCCCACGCTTCCACGGG
AATGGTCTTGGGGAGCGACTTGACGTATTTACACGAGGCTCAGATAATGACTGAGGGTGTGCACGTCAGCCACAAGCAATTGATTAAAATTCTTACA
CAAATTAGCAGTGATGACCCAGACCAGATTTGTTCATGGCTGCTGGCTGTCATTAGTGAGCATCTATTCGGCCAGAAGAGAGGTTGAGGTCTGGTCC
CAGCCATGCCCCTAAAAGTGTCATATCTCAGGGCCTCAGTTTCCCTACTGTCTAAAGGAGAGGTTCAAATGTCAGAAACAGGGGCAGGGTATGGTGG
GACTGGCCTTTAATCCCAGCACTTAGGAGGCTGAGGCATCTGGATCTGAGTGTGAGGTCTGGCTGGTCTCCATAGAGAGTTCTAGGCCAGGACTACA
CAGTGAGACCCTGAGACAGTCACCTAGTAAGTCTCTCCTAACCCTGGGGCAGAGTCATTTCACATCACCCATGAACTGAGAGGGCAAGCTTTGACCT
AACACAAATCCTCCAGAGCTTGGGAAGGTGGGTGGGGGAAGCCAGATCTGAAGCTTGGGTGGAGCGGGAGCACTCTCACCAAGTTTATGGAGGGAGA
CAGGAAAGAGGAGAGCTCCCAGTTCAGGCGGCTGGGGTGAGTGACCGTGGGATAAAAGCCTGGTGGATTATGCTTACCAAGCTTCCCAGAGGTGGCC
TGCATGCGTGGGAGGACCCCAAGAAAGGTCAGCCTGGAAGCAGAGTGATCCTAGAGGCTCTGGCATGGGCCAGTGTGACCCTTGTGTCTGTCCCCTC
CCCTGAGAGGGCTCTGCTAGTGAGTCTGACTCTCAACTGGCTTTTCTTATTTGTCAACCAACAACTCTGGCTTCAGCATCTGTTACCTTCCCCGAGC
AGCTGTGAGGAACGTGGGAAACCACATTGGTTCCTGGAAAAGCAGCTTTCTGTCCATCTATGGATGGCCAGCGCAGGTGTGTTCTCCCAGGGACCTT
GGAGCTGGTGACACACCAGAGCTGGTGGCTCATGTCTGCGCTCTGTAAGGGTGGGGGGGTACCTGACATCACAAGCCCATCTAAGAAGTTCCTTGTG
GGGCTTAGGTTCCATTCCCAGGGTCCCTCTAGAACCATACAGGAGGCGTGGGTTTTCTTTATCAAGGTCAAGGCTGGAAGGAAGACAGCGGCGAGCT
TTAGCGCTTCACGCGCCCACTGCTGAACCTCGCTCCTCATCTGCGCAGTGATGGCAGTATGGGGATCTTGATGGATTTTTATAAAGCTCAAGTTAGT
GGGTGTGAATGCTGCTCTAAGTGTCAGGGTTGCCATCAGCGTTAGCACGGGGTTGCTGCATGGTGATCCCATTGCGTTCTTCTTTCTGCTTTTCAAA
GGGATGGGAGAAAAGTCAGCAAGTAGGAAGGTTGAGGTGGAAGGAAGCAGACCATGACGGCCGCCTTCTCTGGTATTCACTCTGACAAGAGTTAGCC
CTGCACCCCCCAATATCAGATAGTACATACATAGCTCTGGCCGTTTTGAGGAGCTCTTGGTGTGGCTTCTTATCACTCAGCTAGAAGGGCCATTTGT
CCTGACCGACCCCATGTCACTGAAGGTTAGACTTGAGTTAATGGCCTAGATAGACCCAGTGTCTGCTTCCAGTGCTTCGTATGTAGGCTTGCATAGG
CCTCAGCATTGCCAACCCAAGCTTGATACATGGGGCAGTGTGGGTCCAAGATACAGAGCCCATGACTCCACTCCGTCCTTGCCCATGTGGGCAGCTG
AGATCTTCCAGGCAGGGAAGGAGGTGCAGGGTGAGCCTTTGTTGTCCAGCAAATCGCTCCACTCCAATCCTTGCCTAAAAATGATCTCCTAAGAAGA
ATTGGGTGTAGCTGGGTAGTAGAACACTTACACCCCTGAATCAATCTCCAGTACTGGGGAGAGGGGAAAGCAGCCCCCTTTCCTCACATTCCTAAGG
TAGACACTTGCTGTTTTTCTAGTATCATGCAGACATGTTTTTAAGAAGCTATGCCCACAGAGTTCACAGCCTTAATCCCTTCCCATGGGGTGGGGGG
TGGAGGGGGGAGGAGGACTGGACTCTCAACCTCACACTTCCTGATAAGAAAGATCCCTCCCACTGGGATGTCCTTTGCCTCTCGGAGCCCAGGTTTC
CTCCTGTGTGAAGTGATAAGGTCTGTGCCCTGCAGGGCTGCTCAGGAGCCAGACACAGCCATAAAGAGCCTGCTCTGGGCTCCTGGTTTGGAGCCAT
AGGCCAGGAGATGGTTGAGACCTGGTTCCTTCCTGTCCTTTCACCCCCGAAGGCGCTGCTACTGATGGAAGCCAAGGGACAAATGGGCAGGGAGACA
GCAGGCATAGAAGGCTCTGGCTTTTTTCTTGCACAAAGGGGCCACAGAGCAGAGCCCAGCAGGCCACATGCCCTTCTCTGAGAAGAGTTCAGCACAG
TGGTAGCATCCAGTCCTCCCTAAGTGGGGAAGACACCTTCTCCATTGCTTGAGGTTTGTCCACTGTCTCCACTTAGCCACTAGCTTGGTATCCGAGT
GCTGGTAGTTTACAAATATCTGATTTATTCTTTTTATCCCTATGTCGAAAGGCTATGAATCTAGTCACCCCCTCTAACTGGCCCTGTGTCCTGGGGT
CTGTTACTAGTCTTTGTGGGACCTTAGGACATATCCAGGTCGTCTTGCTATGCCTGGTTCAGCTCTGCCAGCAGCACCAGAGATGAGGGTTGGCTGG
GGGTTGGGGGGTGCCTGGGGCGTGGCTCAGTACCTCAGATCCCACGGGATAGGGCTGTACAGAGTGATCATGCTCAGGAAAGCACCCTGGGAGGGAG
TTCACCATAACTAGGTAGAGTCGTTTCTTTTGGACAATTCCACGGCAGTTGTGCGTCTGATGGCAGGCACTAGCCCTGCTGTGACTGGTGGCTCAGA
CTGGCAGGCTTTTCCGTCTCCACGTAGGAAGGGCAATGCTCCCTTGGTTACCTACAGTGGGCTTCCTCAGCTAGGAGGAGGTTTGAGTTTTGTGGAT
GCAGGCTCTGTTCTTCCTGAGGATGGCATGTCCTCCCTCAACAGCAGGCTCTGGGGCGAGGAGGGAGGCAGGGCTGGCAGCGTAGGCAGCCTGTGAT
CTTTGCATCCCAGGGGGTGATGTTGACAGCAGTTATGAGGTTTCTGATTCTTCCTGTTTATTTCCCAAAGGGCACTGGGAAGGGGTAGATTGTGCGG
CTGGATCCATTCATCGAAGTCCCATGAGAAGCAGGATCTAGATCCAGGCCAGCCTTCGGCACTGGCCAGCAAGGAGCATGGACTGTTGCCAGCTGCA
TGTGATAAACGACCTGGACCCAGCCGGCCTCGGACGGACGGGCGGCTTCTTTCTCAACTAACGTCTCTCCCCCTGCTCTGGATGGTGTACGGCTTTA
CAGAGGGGCTTTCTTTATGGTTTTACCAGGGTCTGCTGGGGACAGACTCGAGGCTTACCTTTGCACATGTTAAAGAAAATAAAAATGAAAAAAAAAA
ATCTACCGCAACAGAACAGGCTGGGCTAGTGTGAGCTCTTGGCCTGGTGGGAAGGACAAGACCATGGCGAGATTCTGTGTCCAAGCTGCCCTCTATC
GTGACATTCGAAGATGCCTCTGAGGTGGGAACTGTGAAGTAGGACAGAGCCCCGCAGTCCCCTCTTGTCCGTCGACTCCCATTTAAATTGGACATAC
CTTGTCGTTCTGAGAAAAGCCATAGATAGGTGTAGCTGGGATGTAGTGATGGGGAGGCCCCTGGCCAACAGTGGGAGCAAGATCTTGAGTTTTGAAG
ACCTCAGAGTTCTGGGCGGCCTGGGAAGCCATCTGCAGAACAGAGTTCCTTGTGGGCTCAGTTTTCGCTAGCCCCTGTTCTGCCCTGGAGCGACAGT
CAGATCTCCACGCCCGTTTCTGTTGTTCTACAGTGTCCACCTTTAGTACGCGTTTTTTTTTTTTTTTTCATGATGACCTTGTAAATAGGTCAGATGT
GGAGGCAGGTCTCTTCTGGCTCCATCCACCACACCCAGAAAGAATGGGCTGCTCTGCCCTTCTCAGCCTTGCTAACCAGCAGACACCGAGGAGAGCA
GCGGGGCACGTTAGAGAGCAATCTAACATGGTTGGCAGGTGGGGAGGGTAAAGAGTCCCACTTCCTTTGTGTGTTAGAAGGTGACTACCCTGCGTCC
TTTTCTCCCATTGGCTGAAGTAACCAGAAAGACAAGAGATCCTTAACAAGCCCGTTCTTCCCACTTGTAAAAGGATAGCCTATCTCAGTTCCCAAGG
ATCTGGATTAGATAGATATTCAAAAGAGGCAAGCAGCGAATGGAGGCAGCTCCCAGCTCTGTTCCCGACGCATGATGGTACTGGCTGGGTTTAGTAA
GGTGGGTGGGGCTGCACGGATCAATCCATCAACTCCGTCTTAAGGAGAATCAGAAAGAGGAGATAAAATGGGGGAATGGGGCAGAACAAAGAATTTG
TCCTTTCCCGCTTCTGTCTAGGGTCTGCTAATGCTGGCTTGACGAGGGGTCAGCCACTTCTTTCCTGTTGTGCAGTTGGCTTGCCAAGCAGGCTCCA
GTAGGCCCTTGCCTGCACTCTCTACCATGTGACCATGAGCACTGCTCTAGGGACACCTCCCATCCCTTCCTAGCACCCCAAATGCCCCTTCCCATCT
CTCCTTCCAGAAGTTGGAAATCAAGTCAACTGGATAACGCTTGTGTGAGGACACTTGAGCAGAACGGATACAACAATTTACAAGTCTCTTCATATCT
ATGTATTCTATATTAAAAGTGATAAAGTCATGTTTCCGGGGCGTATTCAAGTAGCTGACAAGTAATTATTTAATAATAGTACATGAGCGCATTGTAA
TTATCCTCGCCATAGTCAGGTAATAGCATCCAATGGGAGGTCCCTACCAACCTGCTGTATCCAAAGTTTTGTAAAAAGTTGTAGAAGTTGTTGATCT
TTTTGATTTTATATTCAAAAAGTCTCTTTTTATAAATATTATTTATTATACAATGTATATACCTTTGAGTTAACTAAGATTATATATTATATAAATA
TATATATATTTGGAGAAAATCTATTTCATCATGCAGTTTTTTTCTGTTAAATCATTAAAGAGAAGGTAAACTTCAAATGGACACATTGTACGATGTG
TGGAGTCTCCAGATTCGTCCTTAGGTCAAGGGTATCTGAATGTTGTGCTAATTAAATGGCCAAATTTACGGTCATTTCCTCTGCTCTCTAAGCAAGA
TGAGGTATGATGGACAGCAGATGTGACATGAGCCTTAAAGCATTACTTGATATTAAGTCAGTATCAAACAAGGAATTGCCAACTTGGGGAAGAACAA
ACCTGGTCAGCAATTCATTCATAGACTTGAAAAATTTCAACAACGCATGTTGATGTCTCAGATCTTTCTACAAGGTTCAGCCCAGGTCAGTGGGTCA
CAGGGCCTGCCATAGTCTTACTCCCTTTCTCACTACATACCCAACTCCAGTTGTTGGAGAAATAGGAATTGCTCAACTGTAAACAAAGCTGGACCAG
AAACAGATAACAGCTGAACTGTGGAAGAGGTTGGTAGGTAGGTTGACCAGATGGTGTTCTGCCTGTGCTCTGGGCCAAGTGACAATGGAATTGTAGG
TTGGTTTAGATAGGTTGCTGGGCAGAGCTCTGGGAAGGGCACTCTCTCCTTGGGCTGGAGTTTCAGTGACTCACCCGAAGCCTTTGCCTGTCATCTT
GATGCTGTGCAGTTTTAGGATGATGGGTGGTCCTTCACCTCTCAGACCTCCCTCTAAAATATCCCCTTCAGTGTTCCTTTCTTCTGGTCTACCACTA
TTAGAGATAACGTATTCACACTGGCCATAGGAACATGAAGCAGTCTGCTTCTGACGGTGGGGACTGCCAACACCTTCTGGCCCTCGGTTGACCATGA
CCGCCTGAGCTTCCATTTGGTGCATCTTTTGCCCCTAGAAATCTTTTTCCACCTATGTGAGGCCCCTTGAGGGTAAAAGCAGAGCTCCTGGCTTCAG
TGCTGGCCATCTTTGACTGCATTTCCCTTGAGCTGAATACTCTTTTTTTTTTCTCGCAGCACTCTGGGTTTTATTTTATTTTATTTATTTTTTTGAC
TGTATAAGGTTGCCAAGTATTTAGATGGTCCCTATCATACTTACCAAGGCCATTTGTCTCCCATGATCATTGTATTCCATTAAAGAGAGTACAATTT
ATAGCTATGCTAGAACACTAGCTGGAAACACTGGAAAGAACAAGTTTCAAGTTGTGCCTAAGTCGTGTGTGCACCTTTTAATTTCAGCATCTGGGAG
GCAGAGACAGGTAGATCCCTCTGTTCTTGGCAGCCAGTGCTACACACCAGACTTAGTGGGTCTTAAAAAAAAAGTTTTGAGAATAATTTGAGAGAAG
ATAAAGGGATATGGTGAGGTTTTTTTTTTTTTATTGAAAGCAACACTATTCTGAAGGCAGGCTGTGGGTAATGGATGGGCACAGAGATAGGAAAAGG
AATCGAGGAGAAAGACAGCAAGGGAAACGAGGCGAGGCTGTTTTGGGTGGAGTGCTGGTCGACCTGTGTAGTGCTGGGCATAACAGCACGTAACATG
GGTGGTGTGGGTCTACATTAGGAAGTGAGTTCAGCAAAACGTATGGTGTTAAGCCATGACTGGCTTAACAGAACTTGGGAGGCAAACAGCTATCTCA
TCACTAATCAATCACCAAATGTTCATTATGGAGACTGTGCTGGAGAATCATAAATACATGTCATTACAATTTCTTGTTTGGACATAGTCTCACTTTG
TAGTGCTGGCTATCCTGGAACCATGCAGAATAGGCTGGCCTCAATCTCGTGCTGATCCCCCTTCCTCGCTACCTCTGGGATTAGAGGTGTGCACCCT
TCCCTCCCGATCTTGGGATTCCTGCGATCACAGGTGTTATCCGTGCTCTGGCATCACGACACACTCACACCCCCTGGGCTGTCATGGGGGCTCAGCC
TCAGCAGTTGTCAGCAAACGAGTGGCTTTCATCTTGCAATAACTTCTAAAATTTAAATGCTGCTTTTGAACTCTTAGCAAACCTTGCCTGGCTTCAC
TTATTTGAAGGCGGCAGGCGCCCAGCTGCCAGGCAAGCAAACTGAAGTCCTGGCCTTGCCTTCTGTTTGGCCAGGTGGATATTTATAGCTGACCCCT
GCTTCAGCAGGCCTGTTGACCGAGGCTTGATTATTAAATGATAACTGAATCTCCTCCATGATTCTGTAACATAAGGTGGGAAATGTAAAGGCTGCTC
ACTGTAATTTTTAGGCAATAAAAAGACAGCCTTAAAAACACATCTGAGGCTCGATAGATGCCCCAGTGTTGGGGAATCGAGGGCGGGGAGGTGGGAG
TGGGTGGGTGGTGGAGGAACACCCTCATAGAAGCAGGGAGAGGGAGGATGGGATAGGGGGTTTCTGGGAGGGGGAGGAAAGTGGAAAAGGGCTAACA
TTTGAAATGTAAATAAAGAAAATATCCAAGAAAAAAGAAAAAAGTATTCAAAATATATATAAAAAAAAAAAAAACCACATCTTAGATTCTAGCTACA
GTTATGCATGTTTTTGGATAGTACATGAAAAAAAAATCCTCAAAAGAAGACAAAAATGTAGTAAGGAAAAAATGAGCTCTTCTTTGGAAGATTTAGT
ATTTCCTGACAGCCGGTGTGTGTGTGTGTGTGTGTGTGTGGGGGGGGGTCATCAGAGCCCTCCACACTGTTCACAGGGCTATGCTGTGGGCTGGAGG
TGGAAGGCCTTTGGGGATCCTCCCAGAACTGGAGGTGGGGGGCAACTGCTGTCCCCCAAATTGCTGGACATGCCTCTTCTCTACACAGCATCCATTT
CTGGGCTTCCTGGTGCAAGGACTGGGGCTGTGGCATCTAGTATCTGAGAGATCCAGAAGGTATGAAGAACAGCAGGGTGAGCTCGGGGGTCTCAGGT
TTGCCTGCTTCAGAGAAGTCATTGTACCGCAAAGCAGCTTCCACCCAGCTCCTGAACAACCGAGCTCATGGCTGCAGTTCAGACTTTTTAAGGTAAA
ATAGGTATCAAACGTAAATATGAAGATTCTCGTACATTAGCATATACTAATGAACTTTTGAGGGGTTTGTTTTGCTTTTGTTTTTGGAGACAGGGTT
TCTGTGCAGCTCTGGTTGCCTGGAATTCACCCTGTAGACCAGGTTGGTCTTGAACTCACAGAGACTCACTTGCCTCTACCTCTAGAGTGCCTGGATT
AAAGGCATGCGCCACCACTGCCCACTGAACTTTTTGTTGTTGTTGTTGTTGTTTGAGAAGAGTCTCTCTGAATGAATGGTTCCCAACCTTGCTAACG
CTGTTTGCCTTTAATGCCGCTCCTATCATGATGACCCTCAACCACAACCTTGCTTTTGTTGGTATTTCATAACTGTAATGTTGCTCCTGTTGTGAAT
TGTAAGGTAAATACCTGTGCTTTCTGATGGTCTTAGGTATAACCCGTAAGTTGAGAACTACTGTCCTACAGGGTCTTGGGACTCACTATGTAGAGTA
GGCTGGCTTCCAACTCAGAGATCTGTCTCCCAAGTGCTGGGATTAAAGGCATGTGCCGTCAGCTTGCATGCGTATCTGTGTGCCATGTGAGTTACAG
TTGTACACTGCCATGTGAATTCTGGGAATTGAACTGAGTCCTCTGGTGAGCGGTAAGAGCTCTTACCCACTGAGCCATCTCTCCAGCCTCCTTTAAT
GTTTTTCTAAAGAGATTTAAAAACAAAACAAAAAAAAAATCTCCTATGCTGTAACAAAGCAAGGAGGGTTTCTCTTCTGTGGCCACTTCGTCATGCA
GCCCCATTAGTGATTGATCCTGTTCCATATAAAATACTGGTCTATACAATATTTCAAGTATATTTACTCTTTATTGCATTCCTTCAATTTGCATTAC
ACAATATAATATCTTTTTCAATATAATTTTGGACAAAAACACAAGACATTAACTTCGTTGAACAAGAAACATAAATTAATTCATGTACGACTTTGTT
GGGAGGAGAAAGGCAGAACCGCAGACAACAGGGACCACGGCGGTGGACAGGGAACCACGGTACTAGCCACGGGAATCACAGTTTCACAACACAATTC
ACTCTTTCAAAGCCACAAAATAAGCCAGGGATGAAAACCCCAAACAAGGTATACAGACGGCAAGACTAGGAGATGATTTGCCTTCTACTGTGGGGAC
AGTGTCAGAGTATCACCCAGGTGCCCCTGCGGCAGCAAGCCTCCCGGGCGGCTTCTGATGGCTGTCTTTGTGTTTGAAGCAGCAGCGGGGAGAGTGG
CATGCGCCCTAAGACTCATCTCCACGAATGACGTGACGACCTAATTGCATTCCTTCTAACCCACTGATTAGGCAAACCACCCTCCAAAGGGCTCGCT
GAGTTCCTCTTCGGGAAGAGGTGTGTTGAGTACGCTGGAATGGATATTCGAGGGCTGAGGGGAGAGGACTAGGGGTGGCTTGTTCTGCTGGAGAGTA
GTGTTTTATTTATGTGCTGCAGACACTACAGATCACTTGAGATACAAGGAGGATGAATTGTAAGTCATCAGGCTGAGGAAAAGGAAGACGATGGCAC
TAATAGGCCTCTAACGTCATCCTAAGGAACTGACCCACTTAGAGCCGGACGCTAATTAGAATTGAGAGGCTTGTAAATTATTTTAATTTTTTTTCTG
TTTAGGGATGTACAGATTTCCATATGTATCTTTCATAAGGTTTTAACGGCATCTACCAAGTGGCTATTATTAGCATATCTCTTACCACAGAGCAAAT
TAATTACACTCAGAGCATATCACAGCCACCCCAGGAATGATTTGACTTCCGATTAGAAAAGCTNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNAAAAACCAAACCCTATTC
ACTAAGAAACACTCGAGGCTTCCTGTTCTCTCTACATTTTCCACCATTTGTTTGCTATTTGATGGGCACTTTCTAAAAGAAAATGGTCACATTCCGG
GCACTTAAAAGCAGGTTCCAGTGAATCATGGACCATCAAGTTTTCTCTGCTCTGTGACAAGGAAGTCTTGTGAAGCAAGCCAAGGAGAGCAGACCAA
CACGACAGCCTGCTTCCTTGCTTGGTCCCTCAGACCCACAGCACAGCGAGGGGCTAGTGCGTGAAAACAAGGCTATACAGGAAGGAAGGAAACCATC
AGCGGGCTCATATGCGCTGGGGAAGACTTGGGACCTCGCAGATGGGTGAGCACTGCGTGTACTTACGCGCCTTCTTCTCTCATGAGGGCCAAGAATT
TCCTCACACGGTACTCAGGATCCATCTAAAAAGCATCGGAAGAAAACAATCAGACGCGCGAAGCTGCCTTTTCTCCACAATGTGCTTCCTCCGCACC
AGGGAGGCGCGTGGTCATGTGCTTCCTGGGTCGTTCAGAACCATGTCACCATTCCCTAGTTGTTGTTTCGAAGCATAATAAAGTGGGTTATGTTATT
TAGAAGCCAGTGGAACTTCCCAACACGGAGAGGCTACTGGTCACTCACAGGCACCTCAGCCAAGCACCCCCCTGCACCCAGTGTTTGGTGTGAGGAG
GAGAAGCACACCAGCCAGGTGTCACCACAACATAGATCGGTGGTCACTCACGGAGGGGTCCAGACATGCCTGGGTGCATTCATGGTGCTATAAATCA
TGAGAGTTTAAATGCTAAAATTCATATATCTTATTGAAGGAGGAGGATAGAACCTGAGAAACCAAACTGAGGTTGATTAAGTGACCAAAATACAGTT
AGAACTTTTTGTTTTGTTTTTCGAGACAGGTTTCTCTGTATAGCCCTGGCTGTCCTGGAACTCATTCTGTAGACCAGGCTGGCCCTCGAACTCAGAA
ATCTACCTGCCTCTGCTTCCTGAGTGCTGGGATTAGAGGCATGCGCCACCACTGCCTGACTAGTTATGACTTTCACATACTCACTAACTGGGCATTC
TTAGCTTTAGTGCCCTTCTGGTGACGCTGCCACACCTAAGAGAGCAGAGAGTGCTGTTGGCCTCCCCTCTCCGTACACCTGGCCTCTGGTAGCCCGA
GAGTCACAGACTCAAGACAAGGTGGTTTGATCCGGGTGATGGTTCAAGTCACCATTACTAATTATCTCTCCTCGCTGGTCATTCTGGATCCTGTTAC
TTCACTTCAAAGTGTATCATAGTTTATTGGCCTAGACTAGGGTGTGTGGGGATCTGGAAAAGAATAATCACAAACAAACATCTCTAATTACTCACTA
TGAATTTGTACTGTAAGAAACACCTCACTTTTAAAGCATGTTGCAGGGCAGAAGTCTGCTGGCAAGAGGGAACTCCCTGCCCTCCAGGAGTGGCTGG
GCTCGCAAGGGCTCAGCTCGCGCAGCCGTGGGCAAGAGTGCACTCAGACTCCCCTCCAAGCAGGCTGGAmGCGACAGTTCTGTCCCAGCAAAAAACA
TGGAACGTAAGTTTCAAATGTATCAAAGTTAAGGGGTCAGACTACAAAGAACTAAGAAAAAGTGTGAACAGCTAACATGAGTCATTTTTTTTTTCCC
CCTCTATGGGGACTAGAATTACACATGAATCAATTGTCAAAGCTTTTTTCTAAAATGGAACTGGCTGGAGCTCGCTGGAGTCAGTACATGCAGGTCT
TCGCCCATTAACTCTATTTAATATGTGTGAGGAGCGACTTACTGACTTGCCTGTAGCTATGGAATTAAGAGTCAAGAGAAAGGGGAGGAAAAAAGCA
ACCTAGATAAATGACTTTATTCCCTCAGGTGACATCCAGTCAGAGCACATCCTTAACACCGAGCAAGTTTTAACAAAATCGAGTCCGGCGAGCAGAG
CGAGTGAGCATGGCTTCCATTCTGCTGAACTGCATGCCTCTCCACTCGGGCTCTGCCCCTGCTCCTGAGGCAGATCTAAAGAGAAGTCACTCAGCTG
CTCGGAATCCATTTGACGTGAAGGGGTTCAAACTCCAGGCCTATGAAGTGTCAGTCGCTCAAGATTAGGCCGAGCGCCCCACTCTCACTTTCTACCT
CACACACATAAGTATTAACTTGATCACTGCGCCGCAGGCATCAAAGGGAGAGGAGGCAGGGGTGGGGAGGAAAACTCGCATAGTTTCTCCTATGCAA
TTATTTTAAAAGATTGAACTGAAACTGGAAAAAGAAGGGCAGAGCTGACTTCTGACAGATTTATCTTTTTTCGGGGACAAAGCCAATACACCTGTAT
GTATGCCTTTACCAGGAATAAGCCTGAGCAGGGTGCTTAGTCTATGACAATAATTTACTACTCAAATGGAAAACTATATAAATTGACTTAAAAACAG
TTGCTGGAAGCTTGGGAAACTTTTGGTTTCTGAAAAACCACAAAAATCTAATTTTAAATTGAGTTTATCAGAGAGAGAGTTAAGATAATTCTCTCCA
TATTCTTATTTATTGGCCTAAGAGCCTCCTTCAGATGTGAAGCTGGGGAACCCCAATGTGAGCAGTGGGCGCGCCAGGGCTCCACAGTGACACACCT
CCTCAGGTCTGGGGATGGCTCACTCCTCATTCATTCACTTCTGCTCCTTTCATTGTATCTAGTGAAATTTATTGAGCTCTCTAATACGTGCTGAGCT
GGTCCAGGGGCCAGCGACAGTGTGTGGCAGCCCAGGGGTCTGAACACACGGAACTCAGTCTACACAAGTGTATTATGTACATGTGTGTGTACACGTT
CGGGCACACAGAGGCAAGGCATTCCAGAAGGAGAGAGGGAGGTGCAGTCCACACAAAGAAAGTTCCAAACCAAATCAGTCTCATCTGCCTGTGCCTA
GCAGCAGGCTGGGAGTCAGTTAGTTTGAGATTTCTAGGGCACTGGGCATGGCGGTATGAGCCTTTAATCCTAGCACTTGGGAAGGCAGAGGCAGGGA
TGGTCTCTGAGTTCGAGGCCAGCCTGGTCTGTATAAAGAGCTCCAGGACAGCCAGGGCTACACAGAGACACCCGTCTGGAAAAACTAAAATAAACAT
TTTTTTAGGAGTTCAGTGAGGGGAAACGCACAGTCACAGCCCGATCTGCACAAGTCGGCCGCATCCACACTTCCACATCTCCCAGAGCTCTTAGCCA
TCAGTGCTGTCACATGCTCACCCTGAAGGACGGAGCCTCAGTAGTGAGAGAGATCGGGGCAGACTCACTGACAGACTCTTCTCCCATCTGCATGACA
CTGGGGACACCTTGCTTCCCTCCTTCCCTTCCAGCCCCCTCTTCATCTTCATTGTCATCCTACAACATCCAGTCTCCCCTCACTGAAGGTAAAGTTT
AAATTATCACCAGGATTTTGCACATCTCCTTTTATTGCTAAGTGCTGTAGATGTAAGCCCTATCCTAAGCTGTTCAGGGTCTCACTCCTTTTCTCTG
ATTGTTAATAAGGACTATGAAATTGACTGTACCCAGGTCACCTCTCTGCAGCCTCCAAGATGTTGAGGAGCCTAGGGCAGGGGTGGGAGTGTCTTTT
CCTCCTTGTTGAATGTCTTCCATGACGGGGCAGTTGTAGTTTTAAATTCGCTTTGAAGCCCTTTCTGATTTTGAGACCTAATGACTCTCTACATTAA
GATTAGAAGTCACGCTCTGCTCTGCTCATTTATTTCTGAAGAGAAAGAGGAGAACATGGGTACTCCTGAGGCCTGGGAGAGGAAAGCAGGCCCTCCT
GGTGTGCTACAGCCATTCCTGTGACTCACGCAGAGACGGGATCGCCTTACCTGCAGGGACATCTCAATCAACATTAGTAACTTCTTGATTCCTATCC
AGACTTTCTTCCCCTTGACTTGCTGAGCAATTGTGGTGCGTTCCTTATCTTTGAAGTTGCCCAAAAGCTATAAAAGCAATAAACAACAATTAGACAC
TTTTATGGTGTGCACACCAACATTCACCTTTAAGTGAACAGTGGGAGGAGCTACCTGCCTGGGATGAATTGACCCACCTAGTCCTGTATGACCATAA
AAGACAAGGTCTTATTCTGTAGCCCAGACTAGCCTCAAACTCATGGTAATCCTCCTGCCTCAGCCTCCGCGGTGCTGAGACTACCTCATCTAGAAGA
GATAAATCTTATGCTGTTTTTCTAAGGGACCATCACTAAACTGAGAGACGCGTTTCCTAGGGAAAACAGCTCCAATCTATGATAAATACAAGTTAAA
CGTGTTAGCATTCCTTCCTGGGCGCAGTTCGTATGTGTCAAAGTACGCTGGTCTTTTCTCTTTCCCAACTACAACAGGGTGTTCAGACAGGTGACCC
AGGCACACTTCTCATGCTGTTGGCGTCTGCTCTGTTGGGAGCCCACGGCAACTCTGAAGTTGTGGAGCCTTTAGGAGCCTCGCCCGACTCGACCACA
CCGAATAACTTACATCACCTTTTCCATCAGATCCCATCTAGTCATCTGTGCACATTAATATTAATCAACACTCACTCGAAGACTCACTTTTTTTAAT
TAAAGGCTTTATCATCTTCAAAAAAAAAAAAAAAAAAAAAGGAAACATTTCCACATTATAAAGTTGTGAGGTTTGGGTAGCGTTCTTTTCTTTTTTC
TTTTTGGTAGTTCCAAGG
MOUSE SEQUENCE—mRNA
CCTCTTCATGATCGCCGGCAAACTTCCTCCTCGGCGCTGCTTCTAATGGAGCCCCACCTGCTCGGGCGTGCTACTCGGCCTCCTGCTCAGTGGACCA
GGGTCCTCGCTGGCTACCCAATTTGGTGGTCCCTGGCCCTGGGCCAGCAGTACACATCTCTGGCCTCCCAGCCTCTGCTCTGCGGCTCCATCCCAGG
CCTGGTCCCCAAGCAACTGCGTTCTGCCGCAATTACATCGAGATCATGCCCAGCGTAGCAGAAGGTGTGAAGCTGGGCATCCAGGGAGTGCCAGCAT
CAGTTCCGGGGCCGCCGGTGGAACTGTACCACCATAGATGACAGCCTGGCCATCTTTGGGCCTGTCTGGACAAAGCCACCCGTGAATCGGCCCTTCG
TGCATGCCATCGCCTCGGCTGGTGTCGCCTTCGAGTCACACGCTCCTGCGCTGAGGGAACCTCCACCATCTGCGGCTGTGACTCACATCAGTAAGGG
GCCACCTGGAGAAGGCTGGAAGTGGGGCGGCTGCAGCGAGGACGCCGACTTCGGGGTGCTGGTGTCCCGGGAATTTGCGGATGCGCGGGAGAACAGG
CCAGATGCCCGCTCAGCTATGAACAAGCACAACAATGAAGCAGGCCGAACGACCATCCTGGACCACATGCACCTAAAGTGTAAATGCCACGGGTTGT
CCGGCAGCTGCGAGGTGAAGACCTGCTGGTGGGCCCAGCCCACTTCCGTGCCATTGGCGACTTCCTCAAGGACAAGTACGACAGTGCCTCGCGAGAT
GGTGGTGGAGAACACCGTGAGTCCCGAGGCTGGGTGGAGACCCTGCGGGCTAAGTACGCGCTCTTCAAGCCACCCACCGAGAGGGGACCTGGTCTAC
TACGAGAACTCCCCCAACTTTTGTGAGCCCAACCCAGAGACGGGCTCCTTTGGTACCAGGGACCGGACTTGCAATGTCACCTCCCACGGCATCGATG
GCTGCGATCTGCTGTGCTGTGGCCGGGGCCACAACACGAGGACGGAGAAACGGAAGGAGAAATGCCATTGCGTCTTCCACTGGTGCTGCTATGTCAG
CTGCCAAGAGTGTATTCGCATCTACGATGTGCACACCTGCAAGTAGTGAGCCAGGGCACTGGGAAGGGGTAGATTGTGCGGCTGGATCCATTCATCG
AAGTCCCATGAGAAGCAGGATCTAGATCCAGGCCAGCCTTCGGCACTGGCCAGCAAGGAGCATGGACTGTTGCCAGCTGCATGTGGTAAACGACCTG
GACCCAGCCGGCCTCGGACGGACGGGCGGCTTCTTTCTCAACTAACGTCTCTCCCCCTGCTCTGGATGGTGTACGGCTTTACAGAGGGGCTTTCTTT
ATGGTTTTACCAGGGTCTGCTGGGGACAGACTCGAGGCTTACCTTTGCACATGTTAAAGAAAATAAAAATGAAAAAAAAAAAATCTACGCAACAGAA
CAGGCTGGGCTAGTGTGAGCTCTTGGCCTGGTGGGAAGGACAAGACCATGGCGAGATTCTGTGTCCAAGCTGCCTCTACTCGTGACATTCCAAGATG
CCTCTGAGGTGGGAACTGTGAAGTAGGACAGAGCCCCGCAGTCCCCTCTTGTCCGTCGACTCCCATTTAAATTGGACATACCTTGTCGTTCTGAGAA
AAGCCATAGATAGGTGTAGCTGGGATGTAGTGATGGGGAGGCCCCTGGCCAACAGTGGGAGCAAGATCTTGAGTTTTGAAGACCTCAGAGTTCTGGG
CGGCCTGGGAAGCCATCTGCAGAACAGAGTTCGTTGTGGGCTCCTGTTTTCGCTAGCCCTGTTCTGCCCTGGAGCGACAGTCAGATCTCCACGCCCC
TTTCTGTTGTTCTACAGTGTCCACCTTTACTACGCGTTTTTTTTTTTTTTTTCATGATGACCTTGTAAATAGGTCAGATGTGGAGGCAGGTCTCTTC
TGGCTCCATCCACCACACCCAGAAAGAATGGGCTGCTCTGCCCTTCTCAGCCTTGCTAACCAGCAGACACCGAGGAGAGCAGCGGGGCACCTTAGAG
AGCAATCTAAACATGGTTGGCAGGTGGGGAGGGTAAAGAGTCCCACTTCCTTTGTGTTAGAAGGCAGACTACCCTGCGTCCTTTTCTCCCATTGGCT
GAAGTAACCAGAAAGACAAGAGATCCTTAACAAGCCCTTCTTCCCACTTGTAAAAGGGATAGCCTATCTCAGTTCCCAAGGATCTGGATTAGATAGA
TATTCAAAAGAGGCAAGCAGCGAATGGAGGCAGCTCCCAGCTCTGTTCCCGACGCATGATGGTACTGGCTGGGTTTAGTAAGGTGGTGGGGGCTGCA
CGGATCAATCCATCAACTCCGTCTTAAGGAGAATCAGAAAGAGGAGATAAAATGGGGGAATGGGGCAGAACAAAGAATTTGTCCTTTCCCGCTTCTG
TCTAGGGTCTGCTAATGCTGGCTTGACGAGGGGTCAGCCACTTTTTCCTGTTGTGCAGTTGGCTTGCCAAGCAGGCTCCAGTAGGCGCATGGCCTGC
ACTCTCTACCATGTGACCATGAGCACTGCTCTAGGGACACCTCCCATCCCTTCCTAGACCCCAAATGCCCCTTCCCATCTCTCCTTCCAGGAAGTTG
GAAATCAAGTCAACTGGATAACGCTTGTGTGAGACACTTGAGCAGAACGGATACAACAATTTACAAGTCTCTTCATATCTATGTATTCTATATTAAA
AGTGATAAAGTCATGTTTCCGGGGCGTATTCAAGTAGCTGACAAGTAATTATTTAATAATAGTACATGAGCGCATTGTAATTATCCTCGCCATAGTC
AGGTAATAGCATCCAATGGGAGGTCCCTACCAACCTGCTGTATCCAAAGTTTTGTAAAAAGTTGTAGAAGTTGTTGATCTTTTTGATTTTATATTCA
AAAAGTCTCTTTTTATAAATATTATTTATTATACAATGTATATACCTTTGAGTTAACTAAGATTATATATTATATAAATATATATATATT
MOUSE SEQUENCE—CODING
ATGGAGCCCCACCTGCTCGGGCTGCTACTCGGCCTCCTGCTCAGTGGCACCAGGGTCCTCCTGGCTACCCAATTTGGTGGTCCTGGGCCCTGGGGCC
AGCAGTACACATCTCTGGCCTCCCAGCCTCTGCTCTGCGGCTCCATCCCAGGCCTGGTCCCCAAGCAACTGCGCTTCTGCCGCAATTACATCGAGAT
CATGCCCAGCGTAGCAGAAGGTGTGAAGCTGGGCATCCAGGAGTGCCAGCATCAGTTCCGGGGCCGCCGGTGGAACTGTACCACCATAGATGACAGC
CTGGCCATCTTTGGGCCTGTCTTGGACAAAGCCACCCGTGAATCGGCCTTCGTGCATGCCATCGCCTCGGCTGGTGTCGCCTTCGCAGTCACACGCT
CCTGCGCTGAGGGGAACCTCCACCATCTGCGGCTGTGACTCACATCATAAGGGGCCACCTGGAGAAGGCTGGAAGTGGGGCGGTGCAGCGAGGACGC
CGACTTCGGGGTGCTGGTGTCCCGGGAATTTGCGGATGCGCGGGAGAACAGGCCAGATGCCCGCTCAGCTATGAACAAGCACAACAATGAAGCAGGC
CGAACGACCATCCTGGACCACATGCACCTAAAGTGTAAATGCCACGGGTTGTCCGGCAGCTGCGAGGTGAAGACCTGCTGGTGGGCCCAGCCCGACT
TCCGTGCCATTGGCGACTTCCTCAAGGACAAGTACGACAGTGCGTCCGAGATGGTGGTGGAGAAACACCGTGAGTCCCGAGGCTGGGTGGAGACCCT
GCGGGCTAAGTACGCGCTCTTCAAGCCACCCACCGAGAGGGACCTGGTCTACTACGAGAAGTCCCCCAACTTTTGTGAGCCCAACCCAGAGACGGGC
TCCTTTGGTACCAGGGACCGGACTTGCAATGTCACCTCCCACGGCATCGATGGCTGCGATCTGCTGTGCTGTGGCCGGGGCCACAACACGAGGACGG
AGAAACGGAAGGAGAAATGCCATTGCGTCTTCCACTGGTGCTGCTATGTCAGCTGCCAAGAGTGTATTCGCATCTACGATGTGCACACCTGCAAGTA
G
HUMAN SEQUENCE—GENOMIC
TGCCTGCCTTGGCCTCCCAAAGTGCTGGAAATACAGGCATGAGCCACCGTGCCCGACCTGGGCAGAACTACATTCTGGATGCATCTTAAACCTCACC
TCAACCCTACACCCTCCCTTTTGTTGTTTCCTTTCTCCCCACAAGTCATCTCAGAATGAGGCTCCCAGCTCCAGGCTCCAGTTGCAACCTCACCCCC
ACCTAGACAAACCCTCAAATCAAGAAAAAGAAGCCCTGGCAGGGCGTGGTGACTCATGCCTGTAATCCCAGCACTGTGGGAGGCTGAGGTGGATGGA
TCGCTTGAGCTCAGGAGTTCAAGACCACCCTGGCTGGGCAACATGATGGAACCCCATCTCTACCAAAAATTTTAAAAAATTAGCTTGTTGTGGTGGT
GTTTGCCTGTAGTCCCAGCTCCTTGGGAAGCTGAGGTGAGAGGATCATTAGATCCTGGGAGATGGAGGTCGCACTGAGCCAAAATTGTGCTGCTGCA
CTCCAGTCTGAGTGACAGAGTGAGACGCTGTCTCAAAAAAAGAGAAAAGAAAAAGAAATCCAAAGGAAAAAAAGAGAGAGACAGAGAGAAAGGAAAA
GAGAAAAGAAAAGAAATTTCTGTTGGCCAGGCCCGGTGGCTCATACCTGTAATTCCAGCACTTTGGGATAGGCCGAGGTGGGCGGATCATGAGGTCG
GCGGATCATGAGGTCAGCGGATCGAGACCATCCTGGCTAACATGGTGAAACCCCGTCTCTACTAAAAATAAAAAAAAATCAGCTGGGCGTGGTGGTG
GGTGCCTGTAGTCCCAGCTACTTGGGAGGCTGAGGCAGGGGAATGGTGTGAACCTGGGGCGCGGAGCTTGCAGTGAGCCGAGATTGCACCACTGCAC
TCCAGCCTGGGAGACAGAGCAAGACTCTGCCTCAAAAAAAAAAAAAAGAAAGAAATTTCTGTCTCAAAAAAAGAGAAAAGAAAAGAAAAAGAAATCC
TAACACTCATCCATCCTTAAGTAAAAGCAACTCACCTTGTAAGTCAGTCTCCACATTTAATCCAACTACCCTACACTAGACATGATGGGTGAGGAGG
ATACAGATAGTTAGGAGGCCTTTGGCTACAAGTAACAGCCTCAAGTGGGCTAACGAACAAGGAAAATGTATTATTTCATATCACAAGAAGTTCCAAA
GCAGGACTCCTCTGAAGGCAGTTAGTGTCTCAGCAATGGTGTCAGAGATGAAGTTTGTTTCTCTCTCTTGGCTCTGTCTTCCTCTGCCGGTCAGCTT
CATCTTCAGGCAGCTTTCCTCATAGTCACAGAATGGCTGCTGCAGCTCCAGGTATCCCAACCCAAAACAACAATGTCCAAAAGCAGGGAAAAGATAT
TTCTTACTGTGTGTCATTTTAAGAGCCCCAAAAACTCTTCCCAGAAACCTTCTGCACCCTTCAGCTGACCTCCCTCAGGCACATGTCAGCCCAGTTT
AGGCCAATCCCTGGCAAGCAGAATGGGTCCCCGTGATTGACTTAGATCAGTCAGGATTTACCTCTGAGCAAGGTAGGGGAGGAGTAAAGACCAGAAC
AAACTGGGGGCCTCTGCCATCCTGGAGGAAGGGACCACCAGCAGTGTGAGCTACACTAAATCTAAACAGAAGCCTGTCTTCCAGAAGTTCAAAAACA
GCAGCTGAGGCTGGGCATGGTGGCTCATGCCTGTAATCCCAACACTCTGGGAAGCTGAGGTGGGTGGATCATTTGAGGTCAGGAGTTCAAGACCAGC
CTGGCCAACATGGTGAAATTCTATCTCTATTAAAACTACAAAAATTATCCAGGCGTGGTAGCTTGCATCTGTAGTCCCAGCTAGGAGGACTGAGGCA
GCAGAATCATTTGAACCCAGGAGGCAGAGGTTGCAGGTTGCAGTGAGACAAGATCACTCCAGCCTGGGTGATAGAGCGAGACTTCATCTCAAAAAAA
AAAAAAAAAAAAGAGCAGCTGATTGCCACAGAGGGAGGGAGTGGATGCTCACCGATCATTTTACATGTCCCAGGTGCCATTTCAAACACAGCAGCTA
TCACTCTGCTGAATAGGCAATTGTCTTTTGAGACAAGTGTTATTATACCCATTTTGCAGTTGATTGAAGCAGGTACAGAGAGGTTAGATAATATGCT
CAGTGTCACATAGTAAGAGGCAGAGCTGGGATTCACTGGGGTTGTGGTAGACCTGGGCTCTAACGCAACCACTCAGAGAGGATTTCCCAGTCAGACA
TGTGAATCCCCATTCAGGCCTTATCTAGAAGCCAAGACAAAGGTGCATAAGAGGTGGGTCCTGCTGGGATGTGGGAAATGCCAGGCAGATGTCCCTG
CTTAGCCCAAGGGCCCTCCCACGTCTGTGCCAGCTGTGCCTAGGCCCTTGCCAGTGAGGCAGCTGTGACCCCATTCCCAGCCCACTGGGCCTCCATC
CATGTGATTAGGCTCCCAGGACTCAGGGCCCATAGCTGGCCACCCTGAGGTCCCAGCAATGACAGCTACTGGCCATACTGGTCTGTGGAGAAGGTGG
TTAGTTGGCAGAGTTCTGGAAGGTGCAAACCAGGACTTCACTAGAAATTTCTTCACCATCTCCAAGGATATTATCAGGCTTGTTCTATTCTATCTCC
CTAATGTGCCCCCACAAAAGTCTTCCCCGCAAGACCCCTGAGATGGAGAGAACTTCCACACCTCTGTTTTGTTTCGTTTTGTTTTGTTTTCTTGAGA
CAGTTTAGCTCTTGTTGCCGAGGCTGGAGTGCAGTGGCACGGTCTTGGCTCACCGCAACCTCCGCCTCCTGGATTCAAGTGATTCTCCTGCCTCAGC
CTCCCAAGTAGCTGGGATTACAGGCATGCGCCACCATGCCCAGCTAATTTTGTAPTTTTAGTAGAGACAGGGTTTTGCCACATTGGCCAGGCTGGTT
TCAACCCCTGACCTCJGGTGATCCGCCCACCTCGGCCTCCCAAAGTGCTGGGATTATAGGCATGAGCCACCATGCCTGGCCTGGTTTTGTTTTTTTA
AGGAAACATTTACGTATTTGGTCCTCTGATGAATGACTGTCCCCAGCAGATGGCAAGTTCCTTGCAGGCCAAAACCTGGGCTGCTTTTGCTCATCAG
TGCTCAGCTGCCAAGGGGCAGCTGCACACAGAGTTGCTCCATGAAGATGCGTTGAAGAAATGGACCAGGTGAAATTCACAGAGCACCTACTGTGTGC
ATCAACCGGAGCTGGGTTATAAGGAAGAAGGCAGACTCCTTGAGGGTTGTCCCATTCCTCGTGGAGCCTTGTCCACAGCAGGCATGCAGTCAATGTG
TGCGATGAGAAATACAGACACTTTCCGCCCTCAGAGCCCAGGCGGAAGGTGGGGGCCCATCGTGACTAAGGTTGGAGACAGAAGAGACCAAAGATGG
CTCCAAGCCCCTCCTGGGTTGCAACCAGGGCTCCCTCCCCTTCCCTGCCTGCTTATGTCCCATACACAGGGGAGTGGAAGGAAAACTCAGGAGCTCT
AGTTCCAGGTGACTGAGAGAAACCGTGGCAGCCACACACAGAGGTCTGGGTCTAACTCCTCAGGTTGGATCACAGCCATGCTAGGAGCTGGCCTTGG
GTTAGAGCCCCAAAAGCCTTGGGCAGGCCAGCCATGTCCAGGGATGGGGCTCAGGGTTATGAGGCAATGGCAGAATAAAGGGGCACAGAGGGAGGCT
GAAGAGAAAGAAAGGCAAGGAGAAAGAGGTGTGGCACATGGCTGGAGAGGCAGCACGAGGGGCCACCAAGCCCCAGATGCCCCCATGTGGCAGTGAC
TTCCCAACAGGCTGCCCACCCTTAATCTCATCCCACTGGAGGCAGCACAGGGTCCTATGAAAGGGACACAGGAACCAAGCGACAGAGCAGGGTAGCT
GGGGTGGGTTGGAGTTGTAGGGGCCAGGGCCACTCAAGATAGGGGCACTGAGGACCCTGAGGGGCCACAGGGAGCCCAGACCTGAGTCAGTGGCCCT
TCTATCTGAGACCTCGGCCCTGCCTGCTCCCTCTCCTCTGGGCCCATCATCAACAGTGTTTTCGAGGGGGTGCTAAGTGCTGGATCCCTCACCACTG
GGGGTTCAGCAGTCTTGTGGAGGGAGTTTCTTTGTGGAGATTTCACAGTTGGAGACGGACAACATATGCAAGAAACTGAGCAGATATGTCTGCTATG
TAGTATGTATAATCACCCCGGCATGGGGGCTTACGCCTGTAATCCCAGCACTTTGGGAGGCCAAGGCGGGCGTACCACCTGAGGTCAGGAGTTCAAG
ACCAGCCTGGCCAACATGGTGAAACACTGTCTCTACCAAAAATACAAAAATTAGCCAGGCATGGTGGCGAGCGCCTGTAATGTCAGCTACTTGGGAG
GCTGAGACAAGAGAATCACTTGAACCCAGGAGGTGGATGCTGCAGTGAGCCAAGATCACACCACTGCATGCCAGCCTGAGCGACAGAGCAAGACACC
GGGCTAGGGAGTGGCAAGGGGTCTGGAGGGGCCTCTCTGAAGTGGATATTTTGAAGTGGGTCTTGGAAGGGGACATCAGACAACATTATCCCGGATA
AATCCTTCTGGAGAAGGCTCCTGGGCGCCTGAAGGGGGACTGCTTAGCAGACACTGACTGGGGCAGGAAGAGAGACCTGAGAGAGAGGCTTACTCCT
TGCCTCAAGAGAAGAAGGAACCCAATATTTTGTATTCACCATCCGCAGTCTGGCATTCCGTCAGCACCATTCTCATTTAACGCTGACACGCTCCCTA
CAAGGTGGAAAGTGTTATCTCCACTCCACAACCGAAGCCAGGGACTCAGAGATGACAGGCAACCGGCCAGTGCCCCCACCCCACCCCATTCCCTCCC
TTCTAAAGGACTTTCACCGCAAATCGCTTTCCAAAATGCTCTGGGGAGGCCAGGCATGGTGGCTTGCACCTGTAATCCCAGCACTTTGGGAGACCAA
GATGGGTGGGTCATTTGAGGTCAGGAGTTCAAGACCAGCTTGGCTAACATGGCGAAACCCTGTCTCTACCAAAAAAACACAAATTAGCTGGGTATGG
TGGTGCATGCTTGTAATCCCAGCTACTTGAGCGGCGGAGGCAGGAGAACCACTTGAATATGGGGAGGTGGAGGTTGCAGTGAGCCGAGACTGCACCA
CTGTACTCCAGCCTGGGCGACACAGTGAGACTCTGTCTTAAAAATATAGGTCAGGAGTTCGAGACCAGCCTGGCCAACACGGTGAAACCCTGTCTCT
ACTAAAATACAAAAAATTAGTCAGGCGTGGTGGCGGGCACATGTAATCCCAGCTGCTTGGGAGGCTGAGGCAGGAGAATCGCTTGAACCTGGGGAGG
CAGAGGTTGCAGTGAGTCGAGACCGCGCCATTGCACTACAGCCTGGGCAACAAGAATGAAATTCCACCTTTGTATATATATAATATATAATATATTA
TATATAAATATATATAATATATTATATATAAATATATAATATATAATATATAAATATATATAATATAATATATATAATATATAATATATAATATATG
TGTTTATTATATAATACATATTTATTATAAATACATATTTATTATAAATACATATTTATTATAAATACATATTTATTATAAATACATATATATATAT
ATACATAAACTGAATGCCCTGGGGAGCTAGAGCTATGACAGGAGGAACTACTGCCACAGGGAGTJTCCTCTTGGTGCCCACAGCGGCCCCCTGTGAT
CTCGGGATCCAGGAAGAACTGGTCACATCCGCCTGCCGCTGGCAGCCTCCCATATGTCAGCGGAAGAGGGAGGCATCCGCAGGGTGGATCTGGCGGT
GCCCCAGTGCGCCCTGGACCGGCATCACCTGGGCCCGCTCGCCCTCCGCCTGTCTGGAGAGGAGAGGGGAGCCTGAAGCCATGCACCCCTTATCCCC
AGCTAAGCTCCGGGATTGCGAGGTAGATTAAGGGCATTCACTCAGGAGGGACCTGCTTAACCAAGATCCTCCGAAATCCGGGCTGCTCACCAGCAAC
CAATTGAGGATGCCGGGCCCGCAGGGAGCGGAGTGGGACCAGGCGCGACCCCTTCCTCGCCTGCTGACCTCCCGCCTTGCTATCAGCACTGCCGCCC
GGGGGCGCGCCCGCGCCGCTGGGCTCTGAGCCGACCTCCGCGTGCACCTGCGCTCCTTTGTGTGTTAGATTAACATCTGGTGCTTACCTACTATGCG
CTTACTCGGTGCAGGTCAGGGGCTTCCTGCATTTCCAGGATGCAGCCTCTATGCCAGCAAACTAGGCTGGGCAGTGGAAAGAAATCCGATTTACTCC
TGCGCCCACCACCCACGCTAGGGCCTTCCTGCTTCCAGGCTCTGCTGTCCAGCCTTCCAGTGGCCTCTAGGGAGCTCGCCTAAAACCGCTCTGCGGT
TCCTACAGCTCCTAGGAGAAAGTTAAACGCCCTCCTCTCCAGGTCAGAGGCCTGGAGCCCCCTATGTGCTCCCTTTGCACTGTTCCTCCATAAGCCC
CTGTGGTGTTAGGCCTTCGGCCCTTCGCACTTCCCCACCTGCTTCCCCGCTGCAACGGCTTCGCCTCTCCCTTTACCCCCCCAGGTTGGTCCGGCTC
CCGACTCCCGCCTGGCAGCCCCAAGACCTGTTTCAAGCAAAAATGAATGGCACAGTGCCTGGATCGGAGATTTCCTCCTCCTTCCCCGCGACAGACG
GGTAGCCCCATGAGGGCGGGCGGGGGCCTGGCCACCGGATTCTTTGTCTACACGGTGCCTCCGCAGGGGGTCCAGTGGAGCCTCACTGAATACACAG
CGGCAGACTTTGAGCCCCGGGGGAGTGCACATCAAAAGGAACCTCCGAGCCAGCTAGAGAGAGGGGTTGGGTTGGGACTGGGGTCCCCTTCCCAGGT
CTAAGGTCCCACACATTCACACACACAACAAAAGTGCTTCCCACGTGCCACCCCATGTCCCCAGGACCCTGGCCCTTGCTGCCTCTCTGCCCTCTAA
TCCAAGAGACCACCCTTCCTTTCAGGGCGATGGCCTCCTGGTCCACCGGCTGATGCCTCCACCCCGCCCTCCCTTCCTGGTCTTGGAGTGAATTTAA
CCCTTTCTCCACCTAGTGAACCACCCTGGGGCTTGGGGTACCTGTTAGAGAGAGGGTGTTTCTATCTGTGATGTGTGGTCTTGGGGCGCCCGCCCTC
CCCTGAGAGAAACCATTCCTGCGTTTCCACCCGCGGCAGCGCAAGGGGTGCAGGAGGAAGGAAATTAAACCTGTGTTGAAGGAAGGACACCTGGGGG
AAAGGAAGGGACTCAGGCTCCCCTTGTCTGAGGAGCTTTCAAGTGGCGGAAATCCCCACACACACCTCCATTCACTAGGGGCCTGGCAGGGCCGCTG
GTAGGTTCCAGTCCTCTCCTCTGTCACTTCCCAGGCGCACATGGCCAGCTCCTAGATTCCACCCTTCAACCCACTTGTTTCCTGCCTGATGCACATG
ACCTGCGTCTGAGTTCTCCAGGCTGCCTGGAGGAGGCATTCAGGTACACATGACCCCTCAGGGAATGATGTTCTTGCCCACAGCACCCAGGATGCTG
GCTGGGCATATCTCAGCCAACACACACTCCCACCGACAGCCTCGGATCTTTTGCTCCCACCTCACAGGGTTGGCACCTGTGGAGTCTTGAGGCCCAG
TAATGACCCCAAGAAGCCATCAAGTCCAGGCCCTGTCGCCCCACCATACTGCATCCACAGTGGTGTCTGAGTGGCCAGGGAAGGTAGCCCTCCCCAG
CTCTCAGCACCACTGTCATGGGGCTCTGGAACTCCCGAGCCCTTGGGCAGGACTCCAGCAGGCAGCTCTGATCAGAAAGCCACCTGGGAGCCCTGTG
GGTGGGACAGGACTCCAGGTAGGACCTAGCAGTCCTCCATCCAAGAGGGAGGTATGTGGAAAGCTCTCCACTGCCAGAGCCTTGGTCCTGAGGCTCT
GCATTCATGGCAATGGTAACCCTCTTTCCAGGTGAATCAGAAACAGGGCACCCCAGAATCAGCCCAGCCATGAGGAGCATCTCTAGGGATCCCCATC
TCTGCAGCCCCCACCAGGTTCACCACTCCTGGTCAGGTTCACGATTTTTCTTCGCCTTGCAGCTCCTTGGTGCTCTCCGCCTCCCCCCTGCCCCTTT
CTTCCTCACACCGGTGGCTCAGCCTCCCCCCGACAGTTTTCTTGGTTTCTGTCTCAGCGTCTCTCTCATCTCTGAGCCCCTCTCTCTCTCTCCTCCT
CTCGGATTCTCCTTTTCCTCCCCTTCCTTCCTCTCTTTCAACCTCCCCTCCCACCCCCTCCAGTCCAGCAATGGGCTGGGAACGCAGCAGGAGCCAT
GACAAGCCCAGGCGGCTCTCCCGACCGTTGGTGCCCCCGAGGCGATTTCCCCGCGCTCCCTGTGCCGGCAGCAGCCGCGTGCGGAGAGGGCTCGCCG
ACCAGAAGGGGCAGCAGGTTGGAGCTGTGGGTACGGGGGGCTCAGATCAAAGGCCAGGAATGTCTGGCCCCCTGGCGTAAGACTTCCTTCAGCCATG
GCCCTCACCTCGTGGAACGCCCGCCGTTCCAGGCCTCTGTGGGACTCGGGTCGGCTCATTCTCTGCCCTTTCTGCCACAGTTCCCTACACAGCGGTC
CCTGCTCCCCACCGGCAGTGCTTCCTTCACCCCAGACCGGGGCTGCGCAGAGTCCTGGTGCCTCAGGTGGCCTGGGAGGGGGCCTAGGGAAAGGCAC
CCAGGTGACCGGGCGTGAGTCCCCAAAACCTGACCATGATGTCAATACCACCCAGGGCTGAGACCTAATTCCTCGCTCTGGAGAGGGAGAGGAGAGG
AAGCAAGCCCGAAGGGGGCCTGGAAGGACTCCTTTCCTGTGGGGAGGGGTGGGTAACTGGAGGCATTTGGTGTCCGCACAGGGCAAGGGCCTTTATC
GCCAAACCCCAGGCTGGAGCTCCCTATCAGTAGAACCGGGGCCTCAGGCGGGACGGTCGGGCAGTGAGGGGCGCTCCGGAGGGCAGCAAGGTCTTCC
AAGGACCTGGGCCGCCCTCGGCCAGAGCTGCGCGTTTCACCGGTCCCTGATGGTCCAACCGAGAATCCCACTGAGGCTGTGGGTCACGCCTCCGACC
CGGGCCAGAGGTCGGGCCTGAACCCCTCAAGGAGGAGAAAGTGACCCGACTCCGAGAGCAGGAAATGCGACTGGCCGCTGGTGTCAGTTGCAGGAAA
TGCAAAGGCAGCAGGAGGTCCCGATACCGATCTGATCCCTGACAAAAGACTCAAATGACACTCGGCTCCGTCCCCCGGCGCCGCCCGCAGCCAGACC
TTCGAGCCTCCGCGGACTTTTCCGCACGTTCCTCGGACACCCGGGAAGTGACCTCCCGCGGCCAGGACGAGAGGCCAGAGAGCGGGCGGACTCGGGA
GGCTGGAAGCTGGGAGGGGTGCGTTCCCCCACCCTCTGATTCCTGACCTGCCCGAGCGGGGCTTCTGGAAACATCCCTGTGCTTTCTACCCTCGATT
CCTCGCGGGCCCACGTCCCAACGGGTCACGGTCCCCGCCAGCAAAGGAGCCACCCGTTCATGCCCACGCCAAGCCCTCGCCATGTGAACGCCCGGGT
CAAGCCCCCGCGCCCTACCCTGGCCAGCGGGGGACCGCGGCGGTAGCAGAGGGTCCGGCCCACCGCGTCCCCCCGAAGCCCCGCGGCGTCCGGGGGG
CGCGGGCTTTGCCCAAGTTTGCTGGGCGATGCCCTTCCCGGAGCCCGAGTTGTGGGCCGGGCGGGAGGGGCGCGTGATTGACAGGCTGAACTACAGA
CTCATCTCTTACCTTAGGCCGCGGGCGCTGATTGGCTGCTCGCTGACATCCTCAAACCCGGCTGCTCCGCGCTGGGCTCGGGAGGGGGGCGGCTCCG
GGTGGAGGTGCGCTTCTGACAAGCCCGAAAGTCATTTCCAATCTCAAGTGGACTTTGTTCCAACTATTGGGGGCGTCGCTCCCCCTCTTCATGGTCG
CGGGCAAACTTCCTCCTGGGCTCCTCTTCTAATGGAGCCCCACCTGCTCGGGCTGCTCCTCGGCCTCCTGCTCGGTGGCACCAGGGTCCTCGCTGGC
TACCCAATTTGGTGGTAAGACTCGCCTCTTGTCTGCCCGCGGCCCGGTTTCTCCGCCCCGTCCGGGGAAGGTGGGGGCGCCGGCTGGGGGTGGGCCG
CTGCAGGGCTGGGGCTCCTCTCCTCGCCTCCCTCCAGCCCCCCACCCGCCCACCCCTCCTGGATTTTCTGCTGGTGTCGCCTTCAGAATCCAACTCC
TGGCTCCTCTGATAATCACCATTTTCCCTGCTCCCCGCTGCAGGGGCTGGAGGACTTTTCCCATCTCAGAAGGTTTCTTATTCTTAGATCCTAACCC
TCTGCCCGTCCCAATCTAGAGGCCCCCGAAGACCCCCCATTTGCTTGCAGTTGGGTTTCTGGAAATCCAGCACTGTCCCTGGGGCCCAGCCACTCCT
CTTCTCCATTTTGGGGTCTTCATGGGCAAAACCATCCTGTTCCTTCACTGCCACCCCACCTCTGCCGGCGCCTCCATCAAGAGATTCCCCATAATAA
CCCCCCTTTGCCTTCCTATTTCAGACCCCAAAGGCTTCAGGGCTCCTCCCGGGGCAGGGGAGTGGGTGGGGGGATTTCATCAGATTCTCCCCACCGC
TCTAGATTCATCTTGAGCCTCTGAAGTGAATATGGTATTTGCTGATCCGGGATGCCATTGAATTCGGATTTTATTCATTTCTTGCTTGTCCACCCTG
GCAAAAGGGGGGTTCCTTGAAAATGTGTTGTCTGGGAGGGGTAGGCTTGTTGTTCTTTGACCAAAAATCCAATTCTCCATGTCACATTTCTTCCTTT
TGGAAAACCGAGGACCGAGGAGAAAGGGAGAGGGAAGGGAGAATTCCGTCCCTGAGAAATGTGGATACAAAAGACATTTTAGTAAATAAGACTGGGA
AGCCGGGAGCAGTGCTTGGGGGGGGCTGTGCGTGTCTGGGGGGCCTGGGTGAGGAATAAACATCGCTTTGGCAGCCCCAGCTCCAGGAGTCCCCTTG
TTTTCCGAGCGGCCCCATCGCCGCTCTCCCACCCTCCGCAGGTCCCAGGGGCCAGAGGGGAAGACACTGAAACCAGTTCTCTGGTTTACACTCCGCC
TCTCCCCAGTCTCCAGCCCCCTACATTCCGGAAAACTTTTAATTAAAAGAATCTTCCCCTCCTGGACATAGGAGAGCCTTGGCCTGGGCGGGGAGGT
GAGGAGGAAGAATTTGGGATCTGTGAGTGAGAAGTGGGCTGTGGCCCCCGGGGTGAAAGTCAGAAAGAAGTGCCACCACGGTGCGGTACCGCCTGCA
GGTCCCCAGACTCCCTGCCTTTCGCCAACCCAGAGTCCAGGGTAGGAGGAGCAGAGACAGCGAGACTTGGGGGCCACACCCCGAGTCTTCCAGTTGT
TTGGGGATGGCAGCATTTGTGTCTGGGTGTTTGGGGCTCCTGGGAGAGAGGGTGGGGACATGGCCAAGGGTCTCTGTTTAGAGGGTGCTAGGAGTTG
CTCCCAGGACAGCCAGCCAAGGGGAAGTCTTTCTTCTGCCCAACCCACACTTTTCCGGGGGGCTGGGCTAACCTGAGGCTTCCAGCACATAAAGAAG
TCCATGGGGTGGCCAGGGCAGCCAGGGACTTCAGGAAGGAGGTTCGGCCAGTCTGGGGAAAGGCTGCTGCCAACACCAGTCCAGCCTCACCCGGCCA
TTCCTGACCCTCTTCCCATTCCCGGGCCAAGGCCATGCCGTTTTTCGCCTTGCTTTCGCAGAGACTCTGGTGGGTTGGGGGAGGGTAGGAAGGAAAG
GTTTGGCGGCCCATAGGTGGCAGGGACTGAGGAAGAGAGACCAGTCTCTCCGGCCCTATGAACTAGAAGAGGGCACTCTTCCAGTTGGTGTAGATTC
TGGTGTGAAATGGTATCTTGGGGACCATCTAGATAGGGATTGCCCAATAGGAAGAAACGCTGGGGAAACCTGGAAGGTCAAGACATTGGCCCTAACA
TAATGCTAGACACATGGCTTGTTTGGGAATTTGGTGGGGGACAGGAATGCTGCTTTGTCTAAGCCAAGGAGAGAGGTGTGTGTGTGTGTGTGTGTGT
GTGTGTGTGTGTGTGTGTGTGTGCGTGTGTTCTGGGTCTATGACCCTAAGAGGTGTTTTCTATACACCAGTGTGTCTCTGTGTGCAAAAGTGTTTGT
GCCTGCAACTGGGCTACTATGGATGTGAAATCGTGTGGGCGTGTTGGGGGATCCATGCCCACGACAGGTTGTGCTGTGTGTGTCTGCACACGAGTGT
GAGCAAGTGGCTGGTGCTGTGATGTGTGTATGTACATGTGTAAGAGACAGAGAGAAGTCTAGAAGTATCTTTGCCCATGTAAAGGTCAGAGCCTGTG
TCTGCCTGTGACGCTGGGCTTGACTGTGTTCATGACTGGGCATGTGAGTGTGTGTGTGCGTGCGTGCGTGTGTGTGAGTGTGTGTGTGCGTGCGTGT
GTGTGTGTGTGTGTGTACGTGCGCGCGCATGCCTTCATGACAGTGTGGGGAAAGCCCCACAGATGTCTTTGTGCATGAATATCAGTGCTCATGGCCA
TGACCTGATTGTGCTTGTATGGAGGAGAGTGAGCCTCTGTCCATAGCTGTGTGAGGCTGTGTCTAGTCCGCCTGTTTTTCCTGTCCCCCAAGGTGCT
GGGCCCAGGGCTGTGTGGCTGTGAATATCTTTGCAGACATCTACCTGTGGGTATGAATGACACCTGAGCTTGGGTGTTTGGTGGGCAGCCATGGGCC
TGGAGGGAAGGTCCAACAGGAAGCAAAAGTCTCACAAGGTGGCTGCAGGGGGCACAGCTGGCCAGGGATGGCAAGCTAGAGAGGCAGGGGCACAGGC
TTCCTTGACACCAGCTTGTCCTGTCTGTCCAGGCACTAGAGTCACTGGCCCTTCTCTCTGAGGTTGTTTTTCTTTGGTCTTGGAGCTGGGGTGGAGG
GTGAGTCACCTTCTCAGGGTTAGGGCTGGGGCTGTAGGGTAGATGGGCCTCTGATTCGTGATCCCATTTCTGCCCCCACTCCCCATACCCAGCCCCC
GCCTGGCAGTCAGATTCCAGTACTTTGAGCATCTAAGCCCATCTTGGCCTGCCCTCTTTGGCCCACCCAGCTCATGCATTCCCCAGGAAGGATTTAC
AGGGGAAGTCATGTAGCTGGGATTTCTGTCCACGCGGACCCTCAAGCATGACACCCTGAGTCCACACTGCTCAGACCCCAGTCCTGACAGAAGGTTG
ACCAGAAGGGACAGCTCTAAGGGGCCTCAGACTGACACCCCTGGAAGGAAGAGATGTTCTCCAGGTAGTGACAGAGCCGGGACAGGAAACAACAGCA
CTGATTTCTCGAATTCCCCTTGTCAGTCCCAGTTAGCTCCTGGTGGAGAAACTTGTCTTTCTTCTTCTTCTTGGGGGCGGTGGGGTGGGGGGATGAA
CCTATGTTATTAGGGCCTAGCCCAGCACCTGCTACATTCCAGCGATGGACGTGCAGATGGAGGGGGCCTGCTGGAGATCAGGAGCTCACCCTGCCGC
TGTGTGACTGAGGACCAGTGGCCACCCATCTGGGTCTGCTTCCTTACTCTTCACCCAGGAGTCCCCAGCCTGCCCTGCCTCACAGCTTGGGTGAAAA
TGTTTTGCAGAGGACAAGGGCAGTGAGCTGGGAAATAGCACACCTGAGTTCTCGCCATCCCAGGACCCATCCTCCCACTCGAGCACCTGCCTAAGAC
CTACCTCAGTTTCCCCATCTGTCAGATGTCCTGCTGCCTCTCCCACCCACTCCCACCTCCACCCACCTTTCAGAGGCTTGTGAACATGTTCAGTCAA
ACCCCGTGGGGGTCAGTGGGCTGGGCCCAGGGAGGCACCTGGAGACTGCGGGGGAGGAAGGCCCCAGGCCAGGTGTTAACACTTGTAACAGGCAGGA
ATTACAGCTCGCTGGGCTGGGCTGGCTGGGGCTGGTGAGGAGCTGAGCTAGCTGGAGT3CTGTTGATCCCACAGACAGACAGACAGACAGACAGATG
GGCAGATACCAGATCGGATGGCCAGGCTTTAACCGGGTGGTCTCCCTTCCAGGGCAGACCTTTCCCTGGGGGGGATATGGCGGGGAAATTAGTCCAG
GTCTCTTGGAAGTTAACAGCCCTCCCGCTCACCCCGCCCCCATTATGGGTGGGGCTTCGGAGTCCAGAGGGAGCAAAATTGGTGGCCGATCTGTTGG
TATTCTTCCGATAGGATCACATTTTGCTTAAAAAGAAGAAAACAGGTCAAACGTTATAATAATACCAAGAGCCTGAGCTGAGTTATGTCCTTAGAAG
AACCTGCTGTAATATGTATGTGGATGGCATGGGGGAAGGGGACGCTCTCAGGGAAAGTTCTAGACTCTCAGAGCATTTTGTCAACAGCTCCGGGGCC
AAGAGGTGAGAGATGGGTGGAGTCTGAGTATCACCCCCACCTCCACCCGAGGGATCCTGAGGGCTTGGGTCTGTGATGTCTGCCCTGCAATTAATCC
TGGCCTGGTGGGAAGACCTTTGGCGATGAGGTCAAAAGAGGCAGGAGCTGGACTTTGGTGCCAAGTCCTTTAGGAGGTGCTTGCTGCAGCTTCACCA
GTGGCAAACCCAGCCAGGCCCTACCAGGGCCCACACTCTGGCATCTGGATACATGCTCAGGTCACTGACAAAGGTGGAGAAACTGAGGCAGACGCGA
GGGCTCTGGGTGGAAATCTCCAAAGAATGCACTCAGCCTAGCAAGCATCTCTCATCCTCCTCACTACAGAACTTAATGCTAATAAACATTAATGGTA
CTTGGGCAGATGTAGAATTGGAAGAAGTCTCTCCTTGGTCTGGGCAGGAGACAAGGAGCCAGGGGATAAGGAGCCAGGGGATGGCACAGGAAAGGTG
ACTTATCACAGAGGAGGATGCAGCTAACAAACCCTTCCAGGGAAAAGGAAGTGGGAAAAAGGAAGATTTTTCTGGTCTCAGAATGTGATGAGCCAGG
AGTTGAGCAGAACCTTTTCCACCTACCCTGATGTGAGCCACAGAAACCTTCTCCAGCTGCCAAGGAGGGCAAGACATCCTCCACCCCTCATTCCTAA
CTCAGGGAGGAGGTATGCCTAGCCCCACCCCCACCCCCCGCCAAAGGTTAGAGCCATCCTGATATTACATCTGTACATCCATGTTATCTTGTATGCT
GGAAAATGGTGAGAGTGGGGAGATGGAGAGACCTATGTCTGAATTCCAAGTTTAATACCAATGGACCCTGTGACCTAGGATGAGTCCCTTCCCTTCT
CTGGGCTTCATTCAAGTCCCTGCCTCCTTCTGGGCTTCATTTTCCTTTACCACATGGAGGGGTCCCTTTTGCCTCTCAGAGTCCCAGGGCTATGACA
GGTTCTGGGCTCGTTGCTGTGAAGCATGACACTCTACTGCACACATTCCGGGCCTGTATTAGACAGTCCACCCGTTTCTTTCTGTGGCTCCATCTGA
CCTAAATCGCAAAGAGCTGGCTGCCCCACTCCTGCCAAGCCAACCTGCTTGCACTTCCAAACCTCGCCCCAAACACTCCCACTCTTCTGGAGCCAGG
CCATCTGCATTCACATCTGGCTTTTTCATGTACTACCTGGGTAACTAGGGCAAGTTAACCTCTGCAAGCCTCAGTTTCCTCATCTGTTAAATGAGTG
TAATGATTGCCTCTATTTCATAGGGTTTTGAAAATGATTGAGTGAATACTTCTTAGAATGATACCTTGCATACAGGAAGCATATCGATGTTAGCTAT
TTTCACCTCCTATCTGCAAGCTATTGGCTCACTGAAGCCCTGGAGCCAAACTTGCGTCTTCCAAGAAGCCCTCCCAGATTTGCCCCACCTGGCTCTG
TTTTCCCCTTTTCTTCTTTTTGCCTGTTTGCCCCACAGTGCTTTAGCTTTATTCTTAAGTTGAGGGTGCTGGGCCAGGCTAATCTCACTTCAGCTCT
TTACCTGCTCAGGAAGGGGAGGGCAGGGTAGAGAAGGTTGCTGGAGTGTCCCCTAATCATTCCCCGACCCCCACAGTCTGCCAGACTCTTGCTGGGA
CCTAGAGCCAGGGGGCTGGGCACAGAAGGGGTGGGGGAGGCAGGCTTCCCAGGGGGAGTGGCACCAGGGCCGCCCAGAGCAGCCTGAGCAGAAGTTG
CTTCTCTAGGAGCTGAGAGATGATTCAGAAGGTGATTTATAGTCGGAAGGCAGAAAGCTTGGCTCCTGGCAGAAGCTGCTGCTTAATTACATTCTTG
GGGCGGGGGTAGGGGGGTGGAGGTATGTGTGGGGAGGCAGCAGGGGCCAGACTTCTGCCTCCTCCCCATCCAGGCCCTCCCTGTCCCTTTGGCTGTC
CCGGCTCTAAGCAGGGTCCTTCTTTATCCCGCTGCCAGAGAGGGAATTCCCTGCTCTCAGTGAGAGGCTAGGCCAGGATTGCAGGAATAGCTTCTGG
GAGTGGAGAAGGTGGGAAAAGAGGAAGGAAGCTGGATCCCATCTCCCGGGACTTCTCCAAGGGAGACTGGATGGCGACGGGCTGGAAAACGTGCCTT
CTGGGAAGGAGCACTGGGTCTCCTCCCTGCTTCTGGGCAGCGGAATAGCTAAGGCACCTGGGCAGAGTTGAGTTTCTTTCTTTCTTTCTTTTTTTCT
TGAACAGAGCCTTGCTCTGTCGCCCAGGCTGGAGTGCAGTGGCACAATCTTGGCTCACTGCAACTTCTGCCTCCCGAGTTCAAGCAAATTCTCCTGC
CTCAGCCTCCCGAGTAGCTGGGATTACAGGTGCGCACCAGGATGCCTGGCTAATATTTGAATTTTTTAGTAGAGATGGGGGTTTCACCATGTTGGCC
AGGCTGGTCTCGAACTCCGACCTCAGGTAATCCACCCGCCTCGGCCTCCCAAAGTGCTGGGATTATAGGCGTGAGCCACCGCACCCTAGCCAGAGTT
TCTAGAATCTTCTGAGAGGCAACTCCACGCTCTCCTTCGGGCACCCTGTCCTGCTGCTGCCTGGGAGGGCCTGCCTGCAGGTTTGCTCCTTTTCTGT
TGCTCAGGTCTCAGTGGGCCATGGGGGCTGGAGAGAAGGTCCTGCGCTAGAGGCTGAAACCCGGTTTCTTCAGCGTTAGGCAGCCTGTCCCTCGTGA
GCACAGTGGCTACCCCAGGTGCACAACGGTTCTGCCAGCAGGCACGGCCCCATACCCTCATGTCAAACTAATATCTCAGAGAGTCACCCCAATGCCG
CTGGGTGCAGCTACAATCACTTCCGCGCCATTGACAGGAGCCCCCATTCTGTTTCCATACCTGCTGAGTTGACATCAGCATTTCCCATATTAACACT
GACCCATGCAACAACCCGCCCGATGACACGTAAGGCCGCTTAGTTCCCTGCATCGCAGCCGTGAATCCCAAATCCCTCACTGCTAGTTCTTAAGGCT
GGAAAGGTGTAGGGCGGGCAGGGGTAGAGTTGCTGAAGCCAGTAGTGTCGATTTATCTGTTTGCAGCCTGGCCTGTACAGCCGGGAGGAGTTGGCAG
GGGTCAGGATGGGGACACTAGACAGCTCTCTGGTTCCCCCTGAGGCGGGCCTTCTCAAAATGGCCTCATTATGTGTGTGGAGTGAATTCCAGAAATG
TCAGGCACACCCATACATCTAGGGAATGAAGGAGATGCCAGAGCGGGCTCTGGGGCGAGAGGGGCCAGTGTGAGGAGGGTGGGCGGCTTCCGTCAGG
GAGTTAGGGCACCTGAGACCTGGCTCTGCCACCATCTTGATATTAGGCAACTTCCTGAACTGTTCTGAGTCTCAGTAACTAGGAGAGTTGGATAAGC
TGCTGGCCAAAGACTTCCAGCTCCGAGATTGCAGTCATGGAGGCTGTATTCCGCGTTCAGGTCACCCAGCTATTTGGACCATCTGTGCCTGGTGGAG
GGGGTGGTCACTGAAAGCTGGTCTCAGGGAAGGGTGGGGGGTGCAGAGCTCTAAACTCTACACACAAAGAGCTGTGAGACAGTGGATGAGTCTCCTC
CCTCCCTCAGTTTTCTCATCTGCAAATTGGATGATGATAATAACACGACCACCTGCCCTGCCTGCTGCAGGGCCGTGAAGAGCTTCAGGGTGGAATA
ATCTTTGTGGGCATGCCCCGTGACCAGTTGGCCTCAGACCTGAGGCCTCTGTGCCCTCTGCCAGGGGCTCTCCATTGAGCATCAGGACCCCTACAGG
AAGGAGCTGAGGACTCCTTCCTGAGGACACTTGGGAAGTGTCAGGTTCTTTCCAGAGAAGTTAACTATCACTGTGAGTCCTAGAAAAGAGGAATAAG
AAGGCTGGGCATGGGGCTTACGCCTGTAATCCCAGCACTTTGGGAGGCCGAGGCGGGTGGATCACCTGAGGTCAGGAGTTTGAGGCCTGCTTGGCCA
ACATGGTGAAACCCTGTCTCTACTAAAAATACAAAAAATTAGCTGGGCGTGGTGGTGGAGCCTGTAATCCCAGCTACTCGGAGAGGCTGAGGCAGGA
GAATCGCTTGAACCTGGGAGGCGGAGGTTGCAGTGAGCCGAGATTGCACCACTGCCTTCCAGCCTGGGCAACAAGACCAACACTCTGTCTCAAAAAA
AAAAAAAAAAAAAAGAAAGAAAGAAAAGAGGAGAGGAATAAGAGCTAGTCCCTGCTCAGACAGTGCTAAGAATGGACCTAAGAGCCCCCCTGAGCCT
CTGAGCTTGAGGAGTTGATATTTCCAGCAGAGGAAGAAGTGGGGCTGGACAGGGGAGTCCTCAGCTCCTTCCTGTAGGGGTCCTGATGCTCACTGGA
GAGCCCCTGGGAGAGGGCACAGAGGCCTCAGGTCTGAAGCCAACTGGTCACGGGGCATACCCAGGTTCACAGCTCAGGGCATGGGGCTTGGTTATCC
TCCTGGCCTAATGCAAAAACCACTGGCCAGACCCAGAGTGAGATTTCCAACCAGGGACACATGGGAAGATGTAGGTGGAGGTGAGGGGGGCGAGGGC
AAGGGCTGCCAGCTGGAAGGGGCTCTGCTGAGGCCTGTAGTCATCAGGGAATCCTAATGGCCTGGGTTAGGCCTAGCAAACTAGGAGACCCATCACA
AGCCATTCCCTCCAGCTCGGCTGGAAAAACTGCGAGGACTGAGTGTGTGTACGTGTGTGTTGGGGAGGGGGATGGGAGCAACAGGACCAAGCACCCA
GGTCTGTCTGGAAGACCTTTCTGGGAAACCTGCCCCTGGGCACAGAGTCCCACTTATCTGCCAGAGCCAGCTGGGCCGGCAAGGAAAACCAATCTCG
CAGGCCCATTAGGAATGCAAATAGGGCTTGTGCTGTGAGTGAGCAGGATCCCGCCTCCTCTCCAGCTCCCACCAATCATCTTCACAAGGTGGGATTG
CTGAGGGTGCCACTCCCCTGCTCTGACACCTCCCATGGCTCCCCTCTTCCTATCTGGTACTAGGCTCCCTATGATTTCACTTCTGTCCACCTGCGCC
AGTTGTAACTTTCACAAACCCCCTCGATCCCCCAATAAACCCACTTCAACTGGAATACTCACCTCTTTGCTTGTTCATTGAAATACTTCCACCTCCT
CTCTCTGACTTTAAAAGGCAGCCCAACCTGGGGCCTGTTGACCTGGGGTCAGTCTGCTCTGCGCTGCTCAGCTCGGACCCCACTTGGCTCAGGTCAT
GTAGCTAACAGCCCTGAACTATGGAGGGTCCTTAGAGGGTCTCTCAAGGTGGATGGGCCCCTCGACCTGAGCAGGCTGCTGTCCCAGGAGTGGAGAT
GAGCCTTTTCTCCACAGTGTCAATTCCCCTGGGTTTCTTTTTCTTTTTTTTTAATCCACGTTCCACTTGTAAACGTTTCCTATTTGTGTACTATTTC
CCCCTCCATGTTCACCAGTTCACACGATGCTTAAGTGTCTAAGTCATTAATTTTAAGATTGCAATTGGCAGAAGTTGTTTCTCTATCTAGGAACAGG
TTAATACGTTTGTTGACCATGTGCTATGTGCTGATCCCCATGCAGACATCATGATTTATCTGAATCTAATTGTTGCCAGTTTATAGATAGGGAGTCC
CACAGTGCAGCCTGGGCATGGCATGGGAAGCCCTGAGCCCACACCCTGTTGGACCTAAAGCCTTTTTCCTTCTACCACCATTCAGGCTCAGTTGTGG
ACTTGTTATTGGGGTAAAAATTTGCTTGTTCATTTTCTACTTTGTTCCCCAAAGGATGGTTTTGAGACAAGTTTACACTTGACCTTCCACTCAAAGG
CTGATGCTTTGGGCTTATAGATACTATTTGTGAACGTCTGGGAAGTCTCTTATTTGGGGATGTGTCCTCTTCACTGGCCACCTTTCCACAAGCCATC
AGGCTGTCCTCAAGCTGGGATTGCACAGTTGCTGACAGCAGTGGCCATGTGAGGATTTTGTTGCCCCTGGGATGGGTAAGGGGCGACCTTGATCAAA
TCACTGCCTCCCGGGCCTCAGTTTCCTCATCTGTGAAACAGAAAGGGTAGCGTAAATGACCTCTCAGATTCCTTCCAGCACTAAGCTCTTTCCTTCT
ACTTAGTCCTTTTTGCTGCTTTGCATCTGGTCTCCATCATATACGTGCTAGAAAGCAGCTGGGCAGAATCTTCTTTCTTGCCCTAGTCCAATTCTTG
TGTTGGTATCTCTGAAACAGCCAATTGTTCATTTCACTTTTACAATTCGACCTTCAGCTTTATTTCTCCCCGAGGAGCCAGCATGCTTTCTCCTTTC
GCTCTTCCGTTTTTATTTTGTTATTGAATCTGATGTGTTTTCCGTCATCAGCCTCTTGAGGGCAGACATGTTGGAGTCTTTGTACCTGGCCTGGGCA
TAATGGATCCTCAATAAAGACTTGTCTTTGGGATACACATTTGCTGTCAACCTCTTCAAATCCTTTCTGGAAAGAAGCAAGGCACATGCGAATTAAA
AAGCAAAATAAACATTGCTCTGGGAGGGAGTTGGTCCTGACTGGATAGTTTTGAGTTATGAGAATTCCAGTCTGCTTCTGTTTCTTTTTTTTCTGTT
TATTTATTTACTTAGAGATGGAGTTTTGCTCTTGTTGCCCAGGCTGGAGTGCAGTAGCATGATCTCGGCTCACTGCAACCTCCGCCTCCCGGGTTCA
AGCAATTCTCCTGCCTCAGCCTCCCAAGTACCTGGGATTATAGGCATGCGCCACCACACCCAGCTAATTTTGTATTTTCAGTAGAGATAGGATTTCA
CCATGTTGGTCAGGCTGGTATCAAACTCCTGACATCAAGTGATCCACCTGCCTCGGCCTCCCAAAGTGCTGAGATGACAGGCGTAAGCCACCAGGCC
CGGCCTTCTTTCTGTTTCTTTACTGTCAGCTTTTCCCCAAGTCTACAAAGGTCTTGTCTTCAGAGAAGTCGCCTCTTTTTTTTCCTCCATGATCTTC
TCCACTCTAAAGCTGAGGTTGGTTCCTAATCTTCCTCTAGGGACTGTCTCTCGTTCCTGGCCAATGACTATGTCCACCCCTACCCCTGGCTCCCTTC
ACAACTGGTTCCTCCCACCCTCCCTTGCCTTGGAAGCCGACGGTGCTATTGCCACCATTCCACAGGTGACGCAGTGCCCAGGTAGCACCCACCCTCT
CTCCAACAGAGCTGCTCCCGTCCCCAGCAGCCATCGTGAGGGCCAAGCCCCACTCTGCAGATGGAGCCCCAAGGAGGAGCAGAGGGGAGCGGCGGCC
AGGGATCTCCCCATTCACCCAGCAAGGTGTCCTCACAACTCTAACCGGCTCTCCCCTCCCCTCCGCTTCTGCCCTTGGGCAACACATGTGCAGGTCA
CCCCACCCAGTCCCTGACTTCGGAGGAGAGAATGTCTAGCCTCAGGCTGGGTGGTTCTCCATCTTAACTGAAGCTTCCATAGGGGATTCTGGAGCCT
TTCTCTGCTACCAGTAAGGATGGAGTCTGTGACCTCTGCACTGTCCTGGAAGCTAAAGCAGCCTCGAGAAAGCAGCCTCTTCATCCCCATGTTACCT
GTGAGAAAACTGAGGCTCAGCTGGGCGCGGTGGCTCACGCCTGTAACCCCGGCACTTTGGGGGACAAAGGCAGGCAGATCACCTGAGGTTGGGAGTT
TGAGGCCAGCCTGACCAACATGGATAAACCCCAACTCTACTAAAAATACAAAATTAGCCGGGCGTGGTGGCACGTGCCTGTAATCCCAGCTACTGGG
GAGGCTGAGAAAGGAGAATCGCTTCAACCGAGGGCTGAAGTTGCGGTGAGCTGAGACCTGGCCATTGCACTCCAGCCTGGGCAATAAAAGCGAAACT
CTGTCTCAAAAAAAAAAAAAAAAAAAAGGAAAAGAAAAAGAAAAAAGAAAAAAAGAAAACCGAGGATCACAGGACCCAACCAGGAGCTGTAAGGAGC
CGGAAGCAAGTGGGCGGCTCCAGTGGCAGCTAGTAATTCCTCGCTTATGATGTCCCfTGGTATCACATACATTGAGGAAACAAAGCACACACATTAG
CTCATCCTCCCGACAACCCCAGGAGGCAGAGACTCTTGTTATCCCCATTCTACAGATGAGAAACTGAGGCCCTGAGGCACCATTCCATTGCCGAAGG
CTCCATGGTTGGTAAACCCCAGAGCTCTAAACAAGAGACAAACTGCCTGAAGCCAGGCCCTTCAGTGACACCACACCACACCACATCCAGGAAGTTC
CTCCTTAGATCTGACTTCAGTCTTTCCTTAATCCAGGCCCAGGTTTTCTTCCTGGGGATGTGAATATCACTGTCAAGTTTCAGTTTGGTTTGGTTTG
AGACAGGATCTTGCTCTGTTGCCCAGGCTGGAGTGCAGTGGTACAATCACAGCCCACTGTAGCCTCAATCTCCTGGGGTGAAGCTATCCTCCTGCCT
TAGATTGCCAAATCACTAAGACTACAGGCAAGCACCATATGTGGCTAATTTTTTTAATTTTTATTTTTATTTATTTTTGAGACAGAGTTTTCACTCT
TGTCACCCAGGCTGCAGTGCAATTGCAAGATCTTGGCTCACTGCAACCTCCACCTCCCCGGTTCAAGCGATTCTCCTGCGTCAGCCTCTCACGTAGC
TGGGATTATAGGCGCCCGCCACCACACCTGGCTAATTTTTGTATTTTTAGTAGAGATGGGATTTCACCATGTTGGCCAGGCTGGTCTCAAACTCCTG
ACCTCAGGTGACCCGCCCACCTCAACCTCCCAAAATGCTGGGATTACAGGCGTGAGCCACTGTGCCCAGCCTTTTTTGTTTTTTTTTGTAGAGATAG
CATCTCCCTACATTACTCTTGCTTGAACTGGACTCAAACAATCCTCCTGCCTCAGCCTCTCAAAGTGCTGGGATTCCAGGGGTAGGGTTCCAGTTTG
CACTTGAGGACCAGGGTTCGTCTCATCCCTGAATCTTCCTGTGTCTGGAGTTGGTTCCTTCCAGTGGGTTCGTGGTCTCACTGACTTCAAGAATGAA
GCCACGTACCTTTGCGGTGAGTGTTACAGGTCTTAAGGACCCCAAGAGTGAGCAACAGCAAGAGTTATCCTGAAGAGCAAAAGAACAAAGTTTCCAC
AGCATGGAAGGCGACCCAAGGGGGTTGGTGCTGCTGGCTTGGGGGTGGGGGGGTGGGCGGGGAGGCAGCTTTTATTCCCTCATTTGTCCCAGCCCAT
GTCCTGCTGATTGGTCCATTTTACAGAGTGCTGATTGGTCCATTTTACAAACCTCTAGCTAGCTACAGAGCACTGATTGGTGCATTCTTACAGAGCA
CTGATTGGTGCATTTTACAAACCTCTTGTAAGACAGAAAAGTTCTCCAAGTCCCCAGTTGACCCAGGAAGTCCGGCTGGCCTCATCTCTCATTCCTT
CTCTAGAACAATTCCTCCCAGGTGACCCATCCTCTACTCACCTTTAACTACTTGTTCTTCCTCTTCTGATCTCTTACCAGCTTTTCTACAACTTCCT
GCAAGTCCTATAGCCACAGAATTGCCTTTAAATCAGGGAAAGAGGGGGCCTGCCATCAGCTCTCTGATTTAGAGGCTTCCGTCTGGTGTCTTACAGC
ACTTCCCTTCCCCATAGGGCAGGGAGTCTGGAATCCAGCATGCCTGCCCTCAAGAGGCTTATGACGGAAACACATCAGATTAAATAACAAAATAAAA
AACAGGCTAGTTGTGGTGGCTTGCGCCTGTAGTCCCAGCACTTTGGGAGGCCGAGGCAGGCAGATCACTTGAGGTCAGGAGTTCGAACCAGCCTGGC
CAACATGCTGAAACCCCGTCTCTACTAAAAATACAAAAATTAGCCGGGTGTGGTGGCACGCACCTGTAGTCCCAGCTGCTGGGGAGGCTGAGGCAGG
AGAATCGCTTGAACCCAGGAGTGGTGGTTGCAGTGAGCCAAGATCACACCACTGCACTCCAGCCTGGGCAACAGAGTGAGACTCTGTCTCAAAAAAA
TAAAAATAAATAACAAAAACAGAAGAGCAGGATCAAGGTCAAGGAGAAGACAAATATGCAAGTCAAGGTCAATGCAGGTGCAAGGATGGAGCTTGCC
CTGAGTTTCCTCACAGCCAGGACATAAGGGAAAACACGCTTTACCTGAAAGGAGAAGGCATGCTGGGTCCTCAGGGAGAAATGCCCGCCTACAGCCT
GTGCCCACCCTCTTCAGCTGCGCCATGAGAATTTAGGCTCCTGGAGTATCTGGCCCAGACAGCCGTGAGCCTGCTTCCGGGCTCTGCTGGCCTCTGC
CTGGGGCCCATTCTGCCAGGGCAGGCCACACTGCCTGTGGCCAAGGGCTGGTCAAGGGAAATTCATTTGCAAGGGCTGTGGCTGGAGCTTGAGGGTG
TGGCTGGGGCCTCCAGCATGGGATGCAGAAGGGATGGGCAGGCTGCAGGCCTGGGGCTAGATCTCCCATGTCACCATTGTCTAGCGTGAACTTTATC
AGGTAACCTAGCCCTCTAGGTGGCAAGTAAGGTTTTGCCAAGATAGGAGGATAGAATGATTGTTTAACTTTTTTTTTTTCTTTCTTGAGACATAGTC
TTGCACTGTTGCCCAGGTTGGATTGCAGTGACACGATCTCACTATAACCTCTGCCTCCCAGGTTCAAGCGATTCTCCTCCCTGAGCCTTGCGAGTAG
CTGGGATCATAGGTGTGCACCGCCACACCTGGCTAAGTTTTGTATTTTTAGTAGAGACAAGGTTTTGCCATGTTGGCAAGGCTGGTCTCAAACTCCT
GAGCTCAGGTGATCTGCCTGTCTGGGCCTCCGAAAGTGCTGGGATTACAGGTGTGAGCCGCTACGCCTGGCCAAGTGATTAACTTTTTAATGAAAGT
ACAGGAAAAGACGTAAACTGTCTTTCCTCTGCTTTCACCCTGCAATAATTGGTACAGAAGACTTCTGTGACCAAATGCGTGCATACACCAAGCAAGC
TGTCAATTCTGCGGCAGACATCAGCCAGCGTCCTCTAATCCAATTCAATTCTGATGATCTCCACCTGGACATAGCATCTGATCCCATAGGTTGAGGT
CTTAGTTCCCAGGACAGCCTCCCACTTCCAGTAGCAATCCCAAGCCCCAGGCTGCTTTACATGTGCTCCTGACTGACCTGCTATAAATCAGGGTTCC
TGTGACTTCCTCCTTGGGTTCCATTAATTTGCTAGAGTGGCTCGCAGAACACAGGAACACACGTTTACTGGTTTATTATAAAGGATATTACAAGGAT
ACAGATGAAGAGGTGCATAGGGCGAGGTATGGGAGAAGCTGCACTGAGCTTCCACGCCTTCCCAGGCACACCACCTTCCAGGAACCTCCACGTGGTT
CCGCTATCCGGAAGCACTTGAACCCTGTCCTTTTGGGCTTGTATGGGGGCTTCATTCCATAGGTATCATTGATTAAACCATTGGCCACTAGTGATCA
ACTTAATCTTCAGCCCCCTCTCCCCTTCCCAAAGGATGGGGTTGGGGTTAGGGTTGGGAGTCCAACCCTCTAATCTTGGCCGGGCACGGTGGCTCAC
ACCTGTAATCCCAGCACTTTGGGAGGCCGAGGCAGGTGGATCATCTGAGGTCAGGAGTTCGAGACCAGCCTGACCAACATGGTGAAACCCCATCTCT
ACTAAAAATACAAAAAAATTAGCCGAGCATGGTGGCAGGTGCCTGTAATCCCAGTTACTGGGGAGGCTGAGGCAGGAGAATCGCTTGAACCCAGGAG
GCGGAGGTTGCAGTGAGCTGAGATCACAACACTGCACTCCAGCCAGGGAGACAGAGTCAAACTCCGTCAAATAAATAAATAAAAGAAAAGAAAAGTC
CAACCCTTTAATCCTGCTGGGTCTTTTGGGGTAACCAACCTCAATCTTGAAGCTGCCTAGGGGCTGCCAGCTCTTCCGTCACGCTCATTAGCATACA
AAAAGACATCACTTTGGAGTTTCTAAGGATTTTAGGAGTTGTACGCTGGGAAACTAGGTCGAAGACCAAATATATATTTTATAATATCACAAACATG
CGTACAGGCGAGCACACAACTTGTCAGTGTACAGCTTAATGAGCTGGATAGAATATCTTATGTTTAACAGTTTGTGAACCTGGCATGAAACTTTTCA
ATACTGGGCACATGGTAGATGAGGCTTCGTTGAGTGGTCTCAATGCTACAGCCAGGCCTTAGGCTTAGGCGGGGAAACAGTTCCACTAGGGTCGAAC
TGGCACAGCATCGGGAAGAAGGCTTGCCTCCTCTAATGTGGCTGCATCATCTCCATCCCTCCCTTGGCAGACCCACAGTGGCACTGCCCTCCCTCAC
AGCTGTGGTCACTTACCTCCAAGTCCCTTTTTGTTAAACTTGGACCATGTTTTAGCCTTTCTCAGCCTCAACGGATCTTCTTTTCTGATCAGTTTAG
TCAGTCTTATTTTTATTTAATTTAATTTAATTCAATTCAATTCAATTTGGTTTTCAAGATAGAGTCTCGCTCTGTTGCCCAGAGTGGAGTGTAATGG
CGCAATCACAGCTCACTGCAGCCTCCGCCTCCCAGGTTCAAGCTATTCTCCTGCCCCAGCCTCCCAAGTTGCTGGGATTACAGGCGCCTGCCACCAT
GCTCGGCTAATTTTTCTATTTTTAGTAGAGATGGGGTTTCACCATGTTGGCCAGGCTGGTCTCCAGCTCCTGACCTCAGGTGATGTGCCCACCTCAG
CCTCCCAAAGTGCTGGGATTACAGGCGTGAGCCACCGTGCCCGGCCATTTGGTCATTTTTTAAACGGTCTAATTAGGAGTGCAGCAGCAGCAGATAT
GACTGTGTTGTTGTATACTCCTCAGCCTGGCCCCTGCCGCCCTGCACCCGCTCCTCTCCCAACCCCAACATGGGTAAGCACATGGGCCTGGGTACAG
CAGTCCTGGACTCTAGATGGTTCTGCCTTTTTATCTGGGTGGACTTCACCTCTCCAAGCCTTGGTTTTCTCTCTGCAAAATGGGGATAACGAGGGCT
ATTGTGAGGATGAAAGGATATAACGCATGTGAGACTAGCACAGGGTCAGGTGTGTACTCAGTGAGTTAGACACACATTTTGCTGACATTAATTTCAT
GTTACAGTCTGCGTTGTTCTCGTGCACACCTCACTTCTCCGTGCCTCTGTGCTACCCCCTGCCTTCTCTTGGGATGAGTACAAACTCCTGTTCGGTC
TTGAAATCATAGTTCAGCCATCACTTCCTCTGCGCAGCCTTCCCTCAGCACCGCGGCCCCAGCAAAGGCCCTTCTGAGCACATCTATTTCATGGACT
AGACTGTAAGTCACCTGTGGGCAGGACCAGTTCTCCATCATCTTTCAACCCATACCACCAAACATAGCACAGGGCACACAGTGGATCCTCAAAGACA
GGTTGTGGGATGAAAGGACACTGTTGAGAGTCACCTGCTGGTCCCAGACTCTTGTCTCCCTGCTCCAGCTGGATTCTCATTGCCTCTGTGATGCTCT
GCCAGGAAGACCAGCTGGTACTTGTTGTGTTTTTTCCTGTATTCTCAGATACCTCTTCATGTGCATCTGTCTTGTCTTCCCAGTGTAAGAGTCAGCC
CCTGAGGGCAGGGATTCTGCTCTGTAGGCCTCTGCATCACTAGCGAGCCTCACACATCATCCTGAGTGTTGCAGGAGCACAGTTAGGAATTCTGTTA
TCAGGCCAGGCATGGTGGCTCACATCTGTAATCCCAGCACTTTGGGAGGCCGAGGTGGGCGGATCACCTGAGGTCAGGAGTTCTAGACCAGCCTGAC
CAACACGGTGAAACCCCGTCTCTACTAAAAATACAAAAATTAGCTGGGTGTGGTGGTGGGCAGCTGTAATGCCAGCTACTCGGGAGGCTAAGGCAGG
AGAATCACTTGAACCTGGGAGGTGGAGGCTGCAGTGAGCCGAGATCGCGCCACTGCACTCCAGCCTGGGTGACACAGTGAGACTCCATCTCCAATAA
ATAAATAAATAAATAGGAATTCTGTTAGCACTTGGCCCAGGAATCAGACTCCTTCCCAAGCAGGCTCCAGTTCAGGCTCAGCTGGTCCCCACTCTCT
GGGACAGCACTCCCCTCACTCTGTGATCTCCCAGGAAGTCCGGCTCTGGGCATCTTACCTTGGTGTGGGGAGGCAACATAGCACTCCAGAAAGAGGG
TAGGCTCTGTGACAGAACCACCTGTGGTGCAAATCCCAGATGTCCACTTCATAGCTTCTAGAACTTTGCTTTGGGTGGTTGCTTAAAAATCTGAGTC
TCAGTCTCTTCCTCTGTATAATGGGAATAAAAAATATGATCTCAAGGTAGTTGTGAGGTTGAAATGAAATAACAGACAGAAACTGGCTAGGCGCAGT
GGCTCATGCCTGTAATCCCTACATTTTGGGAGGCCAAGGCGGACAGATCATTTGAGGTCAGGAGTTCGAGACCAGCCTGGCCAACATGGTGAAACTC
TGTCTCTACTAAAAATACAAAAAGTAGCCAGGCATGGTGGCGCACACCTGTAATCCCAGCTACTCAGGAGGCTGAGATGGGAGAATGGCTTGAACCT
GGGAGGCGGAGGTTGCAGTTAGCTGAGATGACACCACTGCACTCCAGCCTGGGCAACAGACTCCATCTCGAAAAAAAAAAAAAAAAAAAAAAAGTAG
AAATAATAGAAACTGAGTTGAAGGTGAATCATTAAAGATGTTCTACAGGTCATCTAATGTAGCAGGGTTATAAAAATATTCTCTCCCAGGCCAGGCT
TCTTTAGAGCAGAAAGATCTGAATTCCTTCCAAAACAGAAAAGGCTGGCTTCAGGGAATCCCACACACTTTATGTTGGTTTGTCCCAACCAATAAGC
CTATCTGAAGAAAGAAGCGGGTGGGGAGGCATGTGGCAGAAGTCTGTGACTTGGTGGACAAAAGGCAATGCAGACTCTGGCATGACCTTGCGTTCTG
GTATCCTGTTCAGCCAGAGTACTCACAAAGGCCTCCAGTTCTCAGGAAGGGAAGGGTGAATTAAATGTGGTTAGCTATCAGGGATTTTTTTTTTTTT
TTTTTTTTGAGACGGAGTCTTGCTCTGTCACCAGGCTGGAGTACAGTGGTGTGATCTTGGCTCACTGTAACCTCCGCCTCACAGGCTCAAGTGATTC
TCCTGCCTCAGCCTCTTAAGTAGCTGGGAGTACAGGCACGCACCACCATGCCTGGCCAATTTTTGTATTTTTAGTAGAGATGGGGTTTCCCCATGTT
AGCCAGGATGGTCTCGATCTCCTGACCTCGTGATCCACCCGCCTTGGCCTCCCAAAGTGCTGGGATTACAGGTGTGAGCCACTGCGCCCAGCCTATC
AGGGATTTTTATGGGGGTTCCAAGGAGTGGTCTGTGTAGATTTTATATCACCCTAGTTAATGGCTGAGGCACTGGCTGTGTAAGCCCTTGCTAAGCA
CAGTGAGTGAGTGTGGCTGGAAGTGCAGGAGGCCTGGGTGGGATGCAGGCTGATCCAGGGCTGGCAGTCACTGTGTGCATCCAACCTTTAGGCGAGA
GAGGGCTGCTAAACTCTGAGAAGGAATTCAGGGTGAAGAAGAGCTGATGTGCCAATGGTATGGCTGGCACAAGCCTAAGCAGATACCAGCTATTCTC
CTGCAAGCTGATCAAAAATATTTTTXTTACATAATACTTGACATATACAAGAGAACAATCATATATATTAGACATGATGACAGAATGAAAACCTGAG
CCTCACTATGTCCCTGAAGGACCAGACCGTGACTAGGCCGTCGTGTCCATCTGTGGGTCCTCCTCAGCCCATTCCCCATCTCCCTCTAGATGTGACC
ACATCCTGAATTTTACCTATTTATTCCATTACTTAAAAATACTGGTTTTTGGCTGGGCACAGTGGCTCATGCCTGTGATCCCAGCACTTCGGGAGGC
CGAGGCGGGAGGATCACCTGAGGTCAGGGGTTCGAGACCAGCCTGGCCAACATGGTGAAGCCCCATCTCTACTAAAAATACAAAAATTAGCTGGGTG
TGTTAGCACACGCCTGTGGTCCTAGCTACTCGGGAGGCTGAGGCAGGAGAATCGCTTGAATCCAGGAGGCAGACGTTGCAGAGAGCCGGGATCACGC
CACTGCACTCCAGCCTGGAGACCCTGTCTCAAAAAAACAAAAAACCAACAAACAAAAAACTGGTTTTCCCAGGTAATAATGCCTAAATGAAATGTTT
TATTTTGCCTATTTTTAGCATTATAAAAATGGTGTTATACTGCATGTGGCCCTCTATGTTGAGCTATTCTCTCTCTGCATTGTGTTTCTTAGTTTCA
AACTGTTCCTGGATCTTGCTGTAGTTCTCACATGCTCACAGTTGCATTATAGTCCATCTTTAGAATATACAGTTTATCTATTTTGTCTCCTGGCCAT
GGATGTGCATCTCGCTGTGCACAGCAGGGAAAGGTGTTCATGGAGGTACCTGCCTAGGCGTGGAACTTTGGGTTGCAGGATGTGTGAATGTTGAGCT
TTACAAGATTATGCCAAACTGTTTTCCAAAGAGGTTGTGCCCATCTGTGCTGTCATCTTTGAGATTGTTTTTCCCATGGCCCAAGGCCCTTCTTCCC
GCCCGAGGCAAGCCCACTCCTGAGGACTCCACTGCCTTGGGCCTTCACCGTCTTTCTCTCAGGATCCCATTCCCTACTGGAAAACTCTTTCCTTTCC
CTCCTCCACTTGGCCCTGAACTTCCCAGGATTTTTCAAAACCCAGCTTGGAGCTGCCGTCTTCCTGGGAGTCCTCTCAGCCTCACTTCCACTCTCTC
CCAGGTTTTTGGAACAGCTTTTCCAGACAGCTCTTGGGCCAGAGTAGGACATCGCATAGTGTGTTTCTTATTACATCAGCCCCACATAATCCTGTTG
AGAAAGGCTTCTGGAGTCAAATAAACTTAAGAATCGCCACAGGCAACTTGGAGCCTCTTGGTGTTTATTGTTCTCATTTGAATATTAGCAGCTCATT
AGCATAGTTGCAGCACTGAGAGGTCCTGCCAGTGTGACCCGTTTTTATTTGTTTAACTTGTTTTCCAAAAATATTTAACCTTGGAGCCCTTTTCCCA
TTTTCCCCCATTTGGAAAATGGGATCGAGAAGATTCTCCTGCCTATTTCAGTCAGTTTGGTTTTGTGGTCTTATTTTCCTCGGTAACCAAGCTGAGA
GTCCTCTGACTTCTGTTGTAATCATCCCGTGTAAACTAGTTCAGTCCAGACCACCCTTTCTGGAGGGCTTGAGTGGAAGGAATTAATGAATGCACTC
AGGAATTAATGAATGAAGCAGTCAGCCAATCAGTCAGCCATCCAGCTGAGCCCCCTCTCCTATGCAGCAGCCAGGAACCAGGGACTGATCAGGCACC
GCTCATCTTGCACCAGGGGACACCCGGGCTGTCTGGCTGGTGAATCATTGTCCCGTCTTAGGTCCCCTGTAGTTGCATTTTTAGGATCTGTCACCCA
ATGAGGCTGGGAGCCCCTGCGGGGCAGGAACCAAGTTGCGCTTGCCTCTCTGTGTGCCCCGAGTGGGACATACAGATGGGATCTCTCATCCAACTCC
TTCTGAGAGGGGCAGGAGAACATTGTGGTTAAGTCTTCAGCCTCTGGAGCCAGACCAGATGTGTCCCTAATTACCTGGGTAATGTGGGCCAATCTCT
GCCTCAGTTTCCCCACTGAGATAGGGGTGATGATAACATACCTTCCTTACAGAATTATTATGGGGTTAATATGCGGCAAGCACACAAACAGTGCCTG
ATCTACATCATTTCAATTCTTCCTCCCCGCAGCCTCTTTGAAGTGGGGGATGTTTTTCTCATTAGGAAGTTGAGTCCTCCCAAGGAGACGTGGTAAT
GGGTGGTAGCAATAAGATCAGGATTGGGGTCAGCCAGATCACAAAGCCCTGGCCTGCAGGCGTGGAGGGTGCCACCTACCACCTGCGGATGGGACAG
CTCCTCTGTTTCTTTTTACTGATAATTACCCTGCCGACAACCTGATCAGTTCTTTTTTTTTTAATCCTCTATTCATCTCAACAAAATGGAAATCCAT
GGTACTGCAAGGGTTAGAATTTAACATGTGTGCACACGTGTGTGTGCGTGCATGCATGTGTGTGTGTGTGAGAGAGAGAGGGAGAGACAGAGACAGA
GGTACTGCAAGGGTTAGAATTTAACATGTGTGCACACGTGTGTGTGCGTGCATGCATGTGTGTGTGTGTGAGAGAGAGAGGGAGAGACAGAGACAGA
GAGAGACAGAGAGCAAGAGTGCACAAGTGAAGGAGAAGGAGTTAATCTGAGAAGCTGGGGGTTACTAGCACCTTCATGACAGACCAAGGAAGACAGA
GATCCCCGGGTGGGGCTGGCTTTGAGACTGCACACGGCAGAATCGCTTGTGAGTCAGCAGTGCCGCAGAACAGCAGGGTGCTCCCGGAACTGGGCTT
GGTGTCGTCACCCACTGCCCCTTGCACAGGGCCCTGTGTGCCGGGAAATGTTTGCAGGCTGGACACCTGTGGTTGCTTTTAACCAGGCAAGTGAGCT
GGGTTAGAGGTAGCCACATGTTCCTGAGGGCTCGCAGCTTTGGATCTGAGAGGGTTAGACAGGGCGTCAGGGGCTGAGGGTCACTGGAGGAGAATTT
GCTGTCGTCCCTTTGCCCACGGGCCGGCTGGTCTTCTCAGACCCCCAGAGGCCAGCTCCTCTGTTCTTCCCACCTCGGAGGGGAGCCCCAGAAGTCA
CAGGGTCAGCTGTCTGCTTTCCACACTGCCTAGGGCTTTATTCTTGCACCATCCCCAGTTCTGGCCACACCAGGGGAGTGAGACATGGGGCGCCGGT
AGAGGGGCTGAATGCGCTGGTGTGGGGTAGGAGGTTTCGGATGCACATTCCAGAGGGTGTTTATGGGGGAGCAGACAAGCCTGGGGATTCATTCCTG
GTAGGGAACAATTCCCTCAGCACCCGAGGGTGGGAAATGAGGGCCAAAATGGTGGGCTCAGAGGGACACAGGTGAGGTAAGCAGGTAAGAATTCCAC
CAGGTGTCTCAGAAATGTCCTGGGAAGGGCCTGGCATGGTGGCTCACACCTGTAATCCCAGTATTTTGGGAGGCCGAGGCGGGCAGATCACCTGTGG
TCAGGAGTTCAAGACCAGCCTGGCCAACATGGTGAAACCCCGTCTCTTCTAAAAATACAAAAATTAGCCAAGCATGGTGGTGGGTGCCTTTAATTCC
AGCTACTCGGAAGGCTGAGGCACGAGAATCACTTGAACCTGGGAGGTGCAGGTTGCAGTGAGCTGGAATCGTACCTCTGCACTCCAGCCTGAGTGAC
AGAGCAAGAATCTGTTAAAAAAAAAAAAAAAAAGAAAAAGAAAGAAAGAAAGAAAAGAAAGTCCTGGGAAGCTGGTGGAAGGAGGTCGAGATCTTAC
CTGACCTGGGAAAGGAGGGAATTCCCAGGCATCTATAGACCAATAGGACTGTCGAAAGTTTGACTGGTGTGAGAATGGGTTAAGGTGCTGAATTTTA
GATTTTTATCCTCTCCACGGTGGTACAGTGTGTTCTGCAAACAAAGGAGAGCCAGCCTTGAAGAGCCCTTCTCAGAGCTGACTATGTTCTTTGGCTC
CTCAGCCCAGTGAGATGGCCTTAGCTCTGAGCCCTGTGCAATATGCAAACTGGACCAGATGACCCCCAAGGGTCCTTCTAGCTCTGACAGGTCCTGT
CGGGGGATCCAGCCTGCATATAGGGCCTGGGCTTCTTTCAGCCCCATAGATGGTGACCCCTCTTCACCGCTAGTAAGATTGAAGCTCATCAGCAGGA
CCTCAGGCCCTGGCCTCCTTTCTTCTTGGTCCCTTGGGTTTTGGAGTTTTATGTGACATCAATGTCCCAGAAATGCCCATCCTCTTCCTTTTCTCTC
AGGGCACAGTCCCCTGAGGTCTTGTGGTTTATCCACCCCCACCCCTGTTCTATGTCCTCCTTCACTTGCTCTCCCCCACGCCCCTCCCACCAGAGAG
TTTATAAGCTGGCTGTCCCCTTGCTAACCTCCTGCTGTTCCCTATGAACATGCCTGTTAGATTTCACCGTGTGACACTGTCTTGGTTATGTCCCCGT
CGTGCCTAGTTCAGTGAAGAACACATAGCAGGTGCCGAATAAATGCACTCTGCTGGGATAAACTGGTTATCTCCAGAGGGTGGGGTCTGGACAGAGT
GGCAAAATAGCTATAGCATGGGATTTGGGGTCCAACTCCACCTCCACTTCCTGGCCGATGGCAATCTGTCATTGGTAAACTGGAGCCGGCATCTCTA
CTCTCAGGGCTGTTGAGAGGATTCATGGAGAAGCTGTGTGAGGAAATGCTTTGCAGGTTGGAAGGTGCTGGGAACGTGTGTCATCTACCCTGGCCTC
TCTCTCAGGAACTCTTGCCCACATTTATCAGCATCTGCTGGGTGTGTTGTCTTGGGAGAGGTCTTGTTAACCCTCTGCCACCTTGAATTCTCTGGTC
CCCTGAAATTCTTTGTCCTGTCTCACACACAAATGCCTCCAGCCCTCAGGTCGGGGGTCTCCTCTCCTCCTGCAATGCCTTGGGCGTGGGTCCAGCC
TTGTCTGAGACACAGAGAAATGGACCTCCAGTGCGGAAGATCTGCACCAACATTCGAAAGAAAAAAGACTGATAGGCGGGAGCCCAGCATTTGAATT
TTCAGGGATGTTAACCCCAACAGCGGCCGGAGCAGCAGAGCAGGACCTGAGCACTTCCTTCCCTTTCTCCTTAGCCTTCCATGAACATGCTCTCTAT
AAACCCTGTCCTGGTAAATGTCTCCCCTCATTTTTTTTTTTTTTTTTTTTTTTTTTTTAGACAGGGTCTCACTCTGTTGCCCAGGCTAGAGTAAAAT
GGCTCGATCTTGGCTCACTGCAGCCTTGACCTCCTGGGCTCAAGCAATCCTCCCAACTCAGCCTCTCAAGTAGCTGGGACTACAGGTGGTCACCACC
ATGCCCAGCTAATTTTTTTTTTTTTTTAATTTTAGTAGAAACAAGGTCTTGCTTTCTCGGCTAGGCTAGTCTTGAACACCTGAGCTCAAGTGCTCCT
CCCGCCTCGGCCTTTCAAAGTGCCGGGTTTACAGGTGTGAGCCACCTCCCTCAGCCCTCACTGCTTTTTTTGCCATCTCAGTGCCACCATTTCCTAG
CTATGTGACTTTAGACGAGTCACTTGGCCTCTCTGAGCCCATGTCATCATCCGTTAAATTGGGTTCTTTGGCAAATTAAAGGATGTATTCTATGTAA
AGTGCTTTACATGGCAACTGGCACATAGTCAGGCCTCAATAAATGTTAACTATTATTATTAATAATGGCCACTGGCCAGGCGTGATGGCTCACACCT
GTAGTCCCAGCGCTTTGGGAGGCAGAGGTGGGCAGATCACGTGGTCAGTAGTTCGAGGCTAGCCTGGTCAACATAGTGAAACTCTGTCTCTACTAAA
AATACAAAAATTAGCCAGGTGTGGTGGCATGCACCTATAGTCCCAGCTACTCGGGAGGCTGAGGCAGGAGAATCGCTTGAACCTGGGAGGCAGAGGT
TGCAGTAAACCAACACGGCACCATTGCACTCCAGCCTGGGTGACAGAGCAAGACTCTGTCTCAAAAAAATAATAAAATGGCCACTCTGAGTGCCTCA
GTTAATAGGCTCCTTACTACCTTACTTAGAGCAGAGTTGAGGAAATTGGTGAAGTTCTTCACTACCTTATTAGCCACTCTCTACCCAAATAAATGTA
CCCCACCTTATCTTATTCATTATGGGTAATCACCTGTAAAAGCGTTCTTTATTTGATTCACAGCCTGAGTGTCCCCAAAAAGGTGCTCCATGACGTC
ATCCCCCCTCTGGAGGACACCTCCTCCCACCCATGGTGATTTGAGCCTTCTCTTAGGCCAGGGTGATTCCTGGGGGAACCTTAACTGGGGCTAATGT
GCACTGACATTTTGCCTTTTGTGTCGAACACACATCCCATTTGCAGAGATTTAGAATTTTTTGACTTTAAATGTTCGCTATTTCTATGTATGTGTAA
GAGAGAGTTGTTTTCTTTTGTTTTGTTAAGAGACAGGATCTCACTCTGTTGCCCAGGCTGGAGTGCAGTGGTGCAATCACAGCTTACTACAGCCTCA
AACTCCTGGGCTCAAGCGATCCTCCTGCCTCAGTCTCCCCAGTAGCTGGGACTACAAGTGTGCACCACCACACCCAGCTAATTTTTCTGTGTTTTGT
ACTAGACAGCGTCTCGGTGTGTTGCCCAGGCTGATCTCAAATTCCTGGCCTCAGGTGATCCCCCTGCCTCGAAATTCTGAAGGCTGTGATTATAGGC
ATGCGCCACCATGCCAGAGAGAGAGATTTTATCTTTTCTACACAACGTGTGTTTCCCCCAGAAGAAATGCCTTCCTACTTCTTTGATGAATGTAAGT
AGAATGCAGACAGAAATTACAGAAACTCCCTGTGCCCCAGCACCCTTACCAGGGCATGCCTGGCCTGGTGGAGTGACAGGGTGTTGTACACAGTGAG
GGGAAGGAAGGGAGTGGGAGAGGAGGTGAGGCTCTGCAGAGCAGAGTGGACCATTTCTGGTCAGAAAACAGTGAGGAGGCCGGGCGTGGTGGCTCAC
GTCTGTAATCCCAGCACTTTGGGAGGCTGAGGCGGGCGGATCACTTGAGGTCAGGAGTTTGAGACCAGCCTGGCCAACATGTTGAAACCCCGTCTCT
ACTAAAAATACAAAAAAATTTAGCCAGGTATGGTGGTCCTCGTCTGTAATCCCAGTTACTCAGGAGGAGAATCGCTTGAACCTGGAAGACGGAGGTT
GCAGTGAGCTGAGATCACGCCACTGCACTACAGCCTGGGTGACAGGGCGAGACTCTGTCTCAGAAAAAAAAAAAAGAGTAAGGAAAGCGAAGTCCTG
GGTTGGGGGGAAATGTAGCTGCAGGCAAGAACCAAGGAAGGTGTGGAGCCCCCAACCCAGTAGGGAAAAGGCTTGCTGCCTTGAACCCAAAGAGTGT
GAGCATCCTGTACCTTTGCAGACAGAACTTCCACCTACTCACAAACAGATACACCCCACTGCACAGCTAAGAAAGCTAAGGGGAAACCAGGAAGAAG
GGAAAAGAAAAGAGGAAAACCCCAGAGGAAACGAGGAGTCAGGCCTGGGCAGGGGGTTCCAGATGACGCATGAGTCTGCAGGAGGAAAAGGTTTCTT
TGCCAAGGTGGACGGAAACGGAGGGGCGTGCCCAAATGCTTCTTCTTCATGGCTGCTGGATCAGGGAAGTTTTGCTTCTGTGGCCGTGAACATGGAG
ATTTGCTGAGAGGCCAAGGAGAGAGATGATGGGGGAGGGCTACTTCGGGAGAGGAAGTGGACAAGACCAGAGGTTGGAAAACAGGGGACGGGTTGAT
CCTAACAACTTGGAGGGACTGGTACTGGGAATAGGAGGTGGGGCCCAGGGGGAGGGCAGAGGACCCAGGAGCCCCAGCTGTGGACCCAGGGTGGGGC
TGTGGGCAGGTCCCCAGCCCTGTCTGTGACCTAGGGCAGTGCTTCTCAACCTTGAGGGAGGGAGGGAGGATACCACAGAGGGGAGGGAGCACCTAGA
CCACTTAAATCCCTTTCTCTGCAGGGGGCCCAGGCAGCCTTGTTTCTAAGCTCAGCAGGGCCACTTACTATGCAGCCAGGGATGAGAACCAATGGCC
CACCCTGCTGAGTGCTGGAATGTGTAATGAAGTCCTGCGTGGGTCTGTAATGAACCCAGATTCGGCTCCTAACAGGTGCTGAGTGCATGTTTGCAAA
CCCAGGCGCCTGTTGCAAGGAGAGGACAGAGCTAAAGCAAGGAGCCTGCGGAGGAATGGGTGGCAGGGACTGATGAAGTTGGGGGAAGGAGGTGCAG
AGGAAAATGTTGAGGCTCGCTCTGGACTGCCTCGCCTGGGGATGGAGAATGGGGCTGCAAGGGAGGGGCAGCCAGCGCTTTGAACTTGGGTGGGGCC
TCCCTAAATAGAGTGGGGGAAGTCCAGGTGCTGGGCAGGGCTGTCTTCACCTGCCCATCCTCAGCGCCGTGGGGGGATGAGGCGCAAAGAAGCCCTC
CTGGGAATGTGGTGGAAATACCCACCTTGAAAGGTAGAGAGTGGAATCGTTGCCCTGAGCACCTGGGTGTGGTCGCAGCATCCCCCGTGAGGCCTCC
CAACCTGCAGCTGCCTCCTGCGTGCGCATCTGCCACTGCGGCTTTAGGGCCAGGGCCAGGCCCAGCTGGAGGTCCAAGCTGGAGCCCTGGGGCAGTG
TCCAGTGCACTGTGGCCTCAAAGTGCCCAGCCATGGTTTAGTACTCCAGCTGGCAAGGCCCTGGGTTCAAACCCTGCTCTGAATTTGTCCAGTTGCT
TCCCCATGGTGTCATTTGGTTTGTTCCTCTGTCCCTTGTGTTCCCTGATAACAAGAGGTTACCTCTCAAAGCTTGGTTAGATCAGATTAAACGTCTT
TGGCCAAAGAACATCATAGGGAAGGTTGGGGCCTTCCTGCTGCATCATCTCATACATTCTACTGTCACACTTCTGGTGGAGGCTTTTTAATGATCCA
GTTTCCTGGACTCCTCTCCAGAGGCTCTGATCCTGCCACGCTGGTGTTGGAGCCTGGGTGTCTGTATTTTTCAAAACCACCCCAAGTGATCTGCCGT
AGTGTATAGCCTGCATCCCTGACCCCAGTAGCCAGCTGGTCAGGGACTGTCCTTGGTTTTCTGAAGAGCATAGAATAAGAGAGTGGATGGGACAGTC
CAGGTAGGTGCCAGCCCTGGTGGCCAGTGCTATCAGGTGTGAGGCAGGGAGGGTGGCCTCTGGGCCCCCATAAAGAGGAGAATGGGCTTCCTCAGGG
CAGCTCCAGGTTCAGGTAAGAGTCCTTGCAAGACCAGGAGTGCCCCACGCACTGTGCTCCCGGCGTCCAGCATAAGGCCTGGCCCAGCGTGAATGTT
TATTGAATAAAGACACACACCAGGCTGGTGGCTCTCTGAGGAGCCCTGGGGAGCTGCTCTAAGAGTGAGCTCCCAGCCAAGCAGCACAGCTTTGTGT
GTTCCCCACTGGGCTGCTTCTAAGATTCAAACAAAGGACTCCCAAACTGTGGAGCTAGCAGCTGTGTTAAAAGGCCCCAGAGAGCCAGCAGGCCATG
GCCATCACCTGCCCTCAGCTCGCCGTGGAGACAGCCTGTGGGACCTGGGAGAGACGAAGGAAAGGACTGGGCACTCAGTGGGGCTGCTGAGGGCAGT
GAAAGGAGGCAGGGAGGTCCTCGCAGGGAGGTCCTCGCAGGGCCACTGCACCTGCTGAAGCAGGTGCTAGATTCTACATGGAAACGAGTGAAAGAAT
CCCTCCTCTCAAAAAACTCACCATCAATGGAGTCGGTGGACAATTCACTTGAGTGCATTGAAGGTTACCTGTGACGGATGTATAAGAGCTGGGGAAA
TGGCCAGAGGGAACAATTTGAATTGGAGACATTAAAAGGGGATTTAAACATGGGACCTTGAGGGATGAATAGAAGTCTGGAGTCAGAAGCGAACAAG
AGGGCTGGGCAGGCTATGTCTTGTCTTTTACCAGAACTGGCTTGAAGGGTATAGCAATGTGGGGACCCAGCAAAATCCTGGTGTGGGAACCAGCAGA
CTTGGCTGCTCAGAACGGACAGCCTAACTCTGTAAGGTTCTCATTTCCCAATCTGTAAAATGGGAGACTGACTTAGGGTTGGAGGGGTCAGGAAGGG
GAGAAATGTTACAAGGTTAAAATGAATCTAAAGATGCAAACATGGCTTAGGAAAAATTTGAATTAGAATTCCACTTAGTGGAGTGGGAGCAAAGAAA
CAGATGGCCAAGCTCCAGGAGGAAAAGCTGGCCAAGCTGAGCTCAACTTCAAGGCCCAGCTCGAATGTCACCTCTTTGAAGGGGACTTCTTTGCCTT
CTCCCACTTCCTTCCCCTCAGCAGACCTATTCCTTGAAGTCCCAAGTGGGACTTCTCACACTCTGCAACTGACTGGTCACCTGCACTGAGGTGGGGT
CACTGAGGAGGGTGGAGGCTACATCTTTTTTTTTTTTTTTTTTCTGAGGCAGAGTCTCACTCTGTTGCCCAGGCTGGAGTGCAGTGGCACAATCTCG
GCTCACTGAAACCTCTGCCTCCCAGGTTCAAGCGATTCTCCTGTCTCAGCCTCCAGGGTAGCTGTGATTACAGGTGCCCACCACCACGCCTGGTTAA
TTTTTTCTGTTTTTAGTAGAGACAGGCGTTTCGCCATGTTGGCCAGGCTGGTCTCGAACTCCTGACTTCAGTTGATCTGCCTGCCTTGGCCTCCCAA
AGTGCCGGGAATACAGGCGTGAGCCACCGCGCCCGGCCGAGGCTACATCTCATACATCTTGGGCACATAGTATGCAGGTACTGAATGAGGCCCAGCC
TTCAGATGATCAGGTCTGGGGCACTTCAAGAACGATCTGCGTGCTATGAGAGAGACAAGCTCTGGCTGGGAGTCAGCAGGCCTGGGCTGTGCTCTCT
GCACTGCCTCCAACCAGTGGAGGAACAGCAGGGAAGGCACATCGCTTCCCTGGGCCTTAATTTCCCCAAGGAGCTGAACTAGGTGATGTCAAAGGTC
CCGCTTAGCTTTGACATTTTATGACCTAACACTGTAATAGAATCACTAACATGCGTGGCCATTTACAGAATGCTCCTAAAGCCCTTCCAGGACACTC
CTATTGTTCCCCCGCCCCCGCCCTACTCCAATAGCAAGAAAACTTTCACAGGTGGAGACTTTTCTTCAACAGGTCTTTATTGGCTGGGCACGCTGGC
TCACACCTGTAATCCCAGCACTTGGGGAGGCTGAGGTGGGAGGATTGCTTGGGCCCAGGAGTTCGAAACCCAGCCTAGGCAATATAGCAGGACTTCA
TCTCTACAAAAAATTTTAAAAACTTAGCAGAGCATAGTGGCATGTGCCTGTAGTCTCAGCTACTCAGGAGGCTGAGGTGGAAGGACTGGTTGAGCCA
AGTAGTTCAAGGTCTGCAGTAAGCTGTGATTGTGCCACTGCACTGCAGCCTGTGTGACAGAGTGAGACCCTGTCTCAAAAAAAAAAAAAAAAAAAAG
AAAAGAAAAATGGCCTTTACTGAACATCTATCTTTGTACTGATGAGCACCCCCAGAGACACCTGGGCCCACCAGGGTTTCTAGAATGTTCTAGAATG
TTCAGCATCCCCTGAGTTGCCAAGACTAGACATTTAGGGGGAGCTGTATGACAATCACCCAAAACTGATGGGCAGAAATGCTGTGACCCCAAGTAGG
CCTCTGGGCCTTGATGACATCCTCAGTAACAGAGAGCACGGCACCCCTTTGTGTGTCTGAAGGCAGAGGGCTGGGACCCTACCTGAGCACGCACAGG
GCATCTGGGCTTTACCTGGGAGTCCTGAACTGCGCAGCAAAGTGGAGAAACGGACCCAGCTCTCAGCATGTGTCCTTGGGAGCTGCTCCCCTCTGTG
GTCACCAGGCAGCACTTGCCCACCTGTGGGCTTGGTGCCTCTCAGGGAGACGCTCTGGGGTCTGAAGAGTGAGGTCCCTGCCTTTTACCCGTCTCTG
GACACCCCTCTGGGCCTAGGCCCTTCCCTGTGAATCCCCTGCCTGAAGGAAGAGACCCACAGCCCTGGGGTTCCTCTGGGGTGCCAAGTGGGGGACT
GTGTTTGGTCCTGGGGAGGATGGGCTGGGGCTGCTGAGCAGGCCAGGGGTGCAGGACCCCTCCATCCCCCGCTTCTCCTGTCCTCCTCCTTCTTCCA
CCCTTGACTCCTCTCACCTCTTGTCCCCTCTTCCCTCCTGTTTCCACTCTCTTTCCTCCTCCCCAGGTCCCACCAGACTGCTCCATGCCCCGGACCA
CAATTTGGCTGAAGGCTACAATCCCTCTCTCGCACCCTCCTCCCTTTTTCCAGGCTTATCAACTTCACTTTCCTGTGAAATGAAGGGGTGGGGGGGT
GGGTAAGAAAGAAGGAAAAAGCACTTCATGCTCACAACACAACAAAAAACAGGTTTAAACATTTTACAGGTTTCAACATTTTTCAGGCGGTCTTGTC
ACATCTTATCAGATCAGCCAGAGCCGCAGGCAGGCGCTCCAGGAGCTGCTTTGGAGAACAGAAGCTGCAACATTCCAGCCCAGGCACTGCCCCGCCC
TCCACACCCCTTCCCTCCCTTCCCTTTTCTCCCCAAAGAAACATCCCACAGGGCTCCCTGGGATGGAGCACAGGGCCCGCAGCCTCTTCCCTCTGAC
TGAGGTATAGTCTGTGGCCAAACCCTTGGCTTTGAGCTTCTGCCCGCACCCTTTCCTTCTCCCCACGGCTGCCCAGAACCATGGTCCTCCCGGGCTC
TCAGGTGGTCCCTGGCAGCTGAGCTGTGCCACCTCCTCGGAAGGCCCCTCGTGGCCCTGAAAGGGAACCTTCAGAAGCTGCGGGCAGAAGGGAGTTG
CTAAGTCGTCCACAGCATGTAGAAGTGGTTCATTTGGCCAGCAGGGCTAGTGAGTTTCCGGGGCTCCTGGGGCCTGTCCTCGGTGGCTCCTGTCGGA
GGGGAAGGAATAGCTTTTTCCTTGGTTATCTTTTCTCCGTGTGCATGGGTTTTTCTCCTATGGCGTGGATCACTGCCTGGGATATCCCCTTCCACTC
AGTCACCCCCAGACCTCACCTTTCTGTTCCTGCTGCACTCACGGTGACATTAGTGACACAATGCAGGGTGACAGGTCCAGGAAGGGCTGGCCAAAGC
CCAAAGGCTCAGAGTTAAGCCGAGGCAACGTGAGGGACAGACCCAGAACCTGGATCCCAGGACAACAGGGAAGAGCTGGCTTTGTAGCTTGTAACAG
GAGCATGGTTAGGGCTGGGAGGTCCCACTGTGTGGTCAGAGAGGGGTCCCCAAGGGAAGGCAGCTGTGCACTGAAGGCTTAGGATGCATCATAAGAA
AAGCAAAAAGGGGAGGCCGGGCACGGTGGCTCACGCCTGTAATCCCAGCACTTTGGGAGGCCAAGGGGGGCAGATCACGAGGTCAGGAGATCGAGAC
CATCCTGGCTAACACAGTGAAACCCCGTCTCTACTAAAAAATACAAAAAAATTAGCCGGGCATGGTGGCGGGTGCCTGTAGTCCAGGCTACTCGGGA
GGCTGAGGCAGGAGAATGGCGTGAACCCGGGAGGTGGAGCTTGCAGTGACCCGAGATCGCGCCACTGCACTCCAGCCTGGGCGACAGAGCGAGACTC
CATCTCAAAAAAAAAAAAAAGAAAAGCAAAAAGGGGAAAGCGCAGAGGAGGTAGGGGCACAGTGGCTCCCTCTGTGTAGGTATGTTGGCTGTGCATC
TCGTGGGGACCTCGTCCGGAATATGGAAAACACAAGCCAGACCTGGGCGCCTGCTGCCAGTGTTGGCTGGGCAGGGGCTGAGGTTTCGGGAGACCCC
ACCTTTCCAGCCTCCTAGTGTGAGGTTCAGGCCCTGAAGCTGGGCTGGCTGCCCCTTGGCCCCCAGCCCCCTCCCTGGTGCCTCCACCCCTTGGCAT
ATGGCATCAATGCTCCCACCGTGACGCTCAGTGTCCTCCCACCGTGTGCCCACAGCCAAGATGGGGCCTAAAGTATAATTCAACTTTTAGCTCCTTA
CAAGCATCCCCAGATGGATTTCTAGCTTCCGTCCCTCTCTTGGGCCCAGCTCTGTCTCTACGTGGTGCCTGGAAGTCCCTGTGGCCCCACGCACTTC
CTCGTCTTGTTCCCAAAGCTGCTTTGCTTCTTACCTTCCCATCTTGGTGGACACCATCTCCATCTTCCTGGTCATCCTGGCTCAAAACTTTGGCCAT
ACTTTCCTCCTTCCTCCTGTTTCTCATGGGACCAAACGGCTACCAGTCCTTATTCACAGCTTCCTGTCTGTTCCTGAAGCCACTGTCCTGAGATGAC
CAGCGGGCCCATCCCCTGCCACAGACCCCGGTGACCCAGACACATGGGTCTTGGCCCAGCTGCACAATGAGAGCTGAGCCCTGGGAGGTTGCAATGG
ATCCCCAGGCCCCAGAGTCTCCTTAGCATCCACGTCAACCCCGCCTCCCAGCCGCTTTCCCAGGACCCACCTCTTGTCTCATTTTGCACATAACTAA
CAGGATGATATTCCAAGCCCAGCTAAAGGAAGGTGCTAGTTTGTCCGAATAATGTCTGGCACTCAGGGCCCCTTTCTAGCTGCTTCCCCTCCTCTGC
TTCTCTGTGTGGCTCCTAGACCCTGGCTTGGGGAGGCATGCCACCTTGCCTGGGGCTGTCCCCCTGCCACCTGGCTCCACCTGAGGGCCCCTAGCCA
TCCTTCTCCAGCTCGGTAGCTGCTCCACATGGAGCCCTCCAGAGGCAGTGAGGACACACTTGCCCCCGTTCCTAGAGATCGCTCGCCCTGACCATGC
ACCCTCTGGCCCAGAGGTTTACTGGGGCTGAAGTTACAAGTGCCTTGGGACCATGTCTCACTCATTTAAGGCACCCACAGTGCCCAGACCTAGTAGG
TGCTCAAGAAATGCTTGTTGGTCTGTTACTGACAGCAGCCTCCTGGGCTGGGTGCTCCTCAGGTGTGGCTTCATTAATCTCCACGTTCCCAGAACCT
GGAACCCTGCCAGGCTGAGAGTCATCACTCAATTCGTGCGTGTTGAGTGAATAGGCAGTGAATGACGTTGTTTACCAAGGCCAGACTTTAACCTGCT
CTCTGAAGGACAGGCACTTCCACTGACTCCACAAATCACCAGGCCCCAGTCTTGACAGCTGGGGCTAAATCAAGACAGGAGCCCACTGGGCTTAGAG
CCCGGGCAGCTGCTCCTTCATGGTTTTATCTCAAGACCTTGGGTCCCAGCTCAGGGCCTGCACCCACTCTACCCTCACCCTTCCCTTCCTGTCCTAG
ACATATGCAAGGGTGCTCCCACCCCTGCTGGAGATGCAGGCGAGCCAGAGGGCCCCGTTGAGAGAATGAACTAGCCCCGTTGAGAGAATGAACTAGC
CCCTAATGGCAAGGCCCAGAGACATGTCCCACTTCTTTGATGAAAGGCACCCCCTGAGGCACCTTGGCGCTCTCCCACAGCCACAGGACACAGCCCC
ACCTGAGTCCCTTTGGGCTCAGAAGAAACCCCCTACCCTTGGCCCTGCACTGAAATGTCAAGAAGGTCCAGACCTGTGCTCAGTACCCGCAGGGTGC
ACATGTGTTCCAGGAGGGCGAGGAACAGTGGCAGCTGGAATCCTGAGCTAGGTTTCTCTGGTTTTGTCACAAGCTCCCTGCCAAGCAGCCAGGTGAG
CGGCTCCAGTCCAGGCCTGTGGCCTCCCTCATCCACAGCAGTGATTGCCTCGTCACTATGGGTGTAGTTGGGTGCATCAGAGCCCACCAGGCCCAGC
CCCACCGGTGTGCACACATGCCCCTGAACTGGCTAGGCTTCCTGTCCTGACATCTGCCAATCCTGAACGGTTCTTGACCTAAGTGGAAAGGTAGAGG
CTGGGCCCAGGAACAAGTTGGGCCTCTGTCCAGTACTGCCCAGGAGCCAGCAGGTGGAGCACCTGGCCCTGGCCTGTTGTGGGGTTGACAGGAGCAC
CGTGGCCCCCACCACAGGGAGAGGAAAAATGACCACAGGAGAGGTGATTTCTGACTCCTTCTCCTCACCTTCCCTCCTCCTCTCCTCTCAGCAGCCA
TGTCAGTCATTTTAAGAGACACTAGATCTTCAGATCAGAGTCTTAAGATGCCTTCCCTTTGAGGCTGGCACAGATGGTTAGATTTCCGCATGGAGAA
AGGTAAGGGCAAAGGGCCTATTTCTGCAAAGAAGGCATGACTTGGGGAGACTTGGCCCTGGAGAGAGCACCCTGAGACCGGAGGGAGGAGGGAGCTT
TCCCTGGGCCGCAGCCCCTGGGTCCACCTTCTGCAGCCCCACCCATGTGAAGCCAGGTGGGTCCTTTTTGTGCCCAGCCCCAGAGAAGCTTCTGAGA
ATGTGTGCATTCATCACCCCCAGGTTAAAGCCTGGTGGGTAAAAGAACTGTGAGCCCTGGTTCTCCTCCCACCTCCTGGGCAGCCAGCTGTGTGACC
CCGGAAAATGACAAACCTCTCTGGGCCTCAATCCTTCCCGTATAGAGGAAGAGGGAATTAGACTAGACCCGGGCTCTGGAATTTCGTCCTGCACAGG
GGCTCTAGGTGCAAAGTTGAAGGCTGGGGATGCAAGCACCATCCCTCCTTTGATTTTTTAATTTTGCATATTTAACCATTTTAAAGTGTACTATTCA
GTGGCATTAAGTACATTCCAATGCTGTGCGACCATCACCACCGTCTGTTTTCATCATCCCAAACTCTGTACTTATTAAGCAGTAACTTCCCACTCCC
ATGTGTCTGGCTGCTGTCGCTTAGCTTCTTTTCAAGTTTCTTCTATGTTGTAGCTTGCATCAGAATTTCATTCCTTTTTTTTTTTTTGAGACAGAGT
CTTACTCTGTTGCCTAAGCTGGCGTGTCTTTACCTAGAACTTCATGTAAGTGAAATCATACAGTATTTGTTCTTCCGTGTCTGGCTTCTGTCGCTTA
GCTTGTTTTCAAGTTTCCTCTACGTTGTAGCTTGCATCAGAAGCATCAGAATTTCATTCCTTCCTTTTTTTTGGTTGGGGGGACAGAGTCTTGCTCT
GTCGCCCAAGCTGGAGTGCAGTGGCGCAACTGGCTCACTGTAACCTCTGCCTCTCAGGTTCAAGCAATACTCCTGTCTCAGCCTCCTGAGTAGCTGG
GACTACAGGCACGCACCACCAGGCCCGGCTAATTTTTGTATTTTTGTAGAGATGGGGTTTCACCATGTTGGCCAGGCTGGTCTCGAACTCCTGACCT
GAGGTGATCCACCCGCCTCAGCCTCCCAAAATGCTGGCATTACCGCCACCACACCCGCTTCCTTCCTTTTTCTGGCTGAATAGGGTCCCACTGTATG
TATGGGCCACCTGTTGTTTATCCATTCATCTGCTGAGGACTCTTGGGTTGTCTCTACCTTTTGGCTGCTGTGAGTAGCACTGCCGTGAAGACTGGTG
TACAGGTGTCTGTTGGAGTCCCCATTTTCTTTCCTTTTTTTTTGAAACAGAGTCTTGCTCTGTCACCCAGGCTGGAGCGCAGT3GCGCCATCTCAGC
TCACTGCAAGCTCTGCCTCCCAGGTTCATGCCATTCTCCTGCCTCAGCCTCTCTGAGTAGCTGGGGCTACAGGCGCCTGCCACCACGCCCGGCTAAT
TTTTTGTATTTTTAGTAGAGACGGGGTTTCACCGTGGTCTCGATCTCCTGACCTCGTGATCCGCCCGCCTCGGCCTCCCAAAGTGCTGGGATTACAA
GCGTGAGCCACCGCACCTGGGCGGAGTCCCCATTTTCAATTCTTTTGGCGTATACCAGGTGTGGCATTGCTGGATTATAGGGGTGATTCTATGTTTA
ACTTTTTGAGGGACAGGGCCCTCTTTTCATATGTTCTGTATGTAGGGGAGAGGAGGGAGATTGTGTGTGATTCCATTTGGGAGGCCACCTGGTTGGG
TGCAGGACTGCCTACCCTAATGGATCACTCAGGGGAGTGGGTGCTGGCAGCACAGTGGGTGTCCCTGGCAGCACAGTCAGACCCCCATCAGCAGGGG
ATTGGCCGTCACTGGGTGGACGGCAGCATCTGAGGCCAACAACCCCAGTGTCAGATCAGGCAGGACGCACTGTACAGGGTGCCTTTAGGTGTCTCAC
CATATGTTAGGTGCGTAAGTCAATGCAGCATCCAGGCTGACCTGAATTCACTGATTAAAAATGTTCTCTTAAAATGTTCTCTATGGGCAGGCATGGT
GGCTTACGCCTGTTATCCCAGCACTTTGGGAGGCAGAGGTAGGTGGATCACTTGGTCGGGAGTTCCAGACCAGCCTGGCCAATATTGCGAAACCCTG
TCTCTACTGAAAATACAAAAATTAGCTGGGCATGGTGACGCATGCCTCTTATCCCAGCTACTTGAGAGGCCGAGGCAGGAGAATCACTTGAACTCGG
GAGGCAGAGGTTGCAGTGAGCCAAGATCACGCTTTGTACTCCAGCCCGGGCAACAGAGCAAGATTCTGTCTCAAAAAAAGAAAAAAAAGGTCCTCTC
TGCCTTGTGCTCACCATGCAGGCCTCCTGCAGCCCTCTGGCCACAGCAGGCGTTATCAGGGTAGAGGCACAGAACTGGGGTGGGGATGGGAGTTAGA
TAAGGCCATGCACAAGGTTTAGGTCTTGCCTATTTGTGGAGGGATATTTCACTCCAAGGGGTCAACACCAAAGTTTCCACAAGCAGCCCGTTGTGCA
GGGCACCCGCAGCCCATCCGCCTGGCTGCCCGTGTGTGTCCTTGGCCCAGCTGGGCAGTGGGTGGTCGGAGTGGAGGATGAGCCGGCTTCAGGAGGA
GGGTGGCAGGGTCCTAGGGAAGGGAAGGGCCGGGCCAGGAGCTGGGTATAAATAGCCCCTGGCCCAGCTGCGAAGGCCTGGCAGGGTCCTCCGCCCT
CAAGGAGTCTTGTCCTGTGTGTAGGCGGAAGCTCCGGATGCCGGGAGGTCTGGCGAGTGAGGGGCATCAGGCTCGTCCTTTGGAAGAGTTCTTCATT
CCTTTCCTCCAGAATCATGAATACCTAGCATGTGCCCGGCCCTTTGGAAGACCGAGTGGGCTGGGGTGTGTGTGTTGTGTGTGTGTGTGTGTGTGTG
TGTGTGTGTGTGTGTGTGATGGGGTAGGGACCGGGGAATAACTGGAGAAAACACTACCACTACCTCTCTTCTTGGAGACTGAGACTGGCATAGCCAG
ATGGAATTGGAAACAAATCAGTTCAGCACTTTGGGATAAGAAGGTTTGAATTAGGTACAGGAAAGATTCAGGCGGAGGAGAGGTGACTTCTGTCTGG
AGCTCACAGAAGGCTTTAGAATGGTGGTGACCTAGGAATCAGGTTTCGAAGGATGGTGTAGAATGGTTGCCGGGGGCCGGGCAGTGGAGAGGTGCTT
CGAGCCGAGGGCTCAGCATGTGCAAATGTGCAGAAGTGTGAAACAGCCCCGCTGGAGTCATTCAGCTGGCTAGGCAAATCCGTCAAAGCCACGCACA
CTTTGGCACAAAGAATCGAGGAGCATGAGAGTCACTGTGCCGCGTCCCGAAGTGGAACGTGTACTTCAATAATGGGGAATAGGAGTGGCGAGTTAAA
GTAATGTGTCTGCGATGGTGGAATTTCAGCAGTGTGCGGGCAGGAGGCTCAGGGTGGGGGTGGCCGGGCATGTTTGGGGAAGGGCAGCCATCCTGCA
GAAATCAATCCCATGGCCACAGCCTCTTTAGTCTCCGACACTGGCCCAGGAGCTAAGCCCCATCGCCAGAGGCCTTGCTAATTACTCGCAGGATGAC
CCCATCTGAACTGAAGCCTTTGAATGGAGCATCCTGAAAATGTCCTTTTGCCTGCACTCTTTGAGTGACCTTGAACCCTCATCCAGATCACAACTGT
CTTTGTTCTGCCACCCCTGTGAGGACTGAGGTCTWTTCCAGGCATCCTTTCCAGTGTTGGATAGGAAGGGGCTGAGATTTCTGATGTGTTCTACCAA
GTCAGGGATGCAAGGATGGGAGAAATGAGGTTGGTGACAAGGCAGGGAACAAAGTGCTGGGGACAGCTCCACACACACCGTAACCATAGCAACCACC
ATCTAGCAAGGGCTCCCCACGTGCCATGCACTGGATTGTGTCACTTATTCCTCCTGTCGTGTCTATTTTACCGATMTAGAAACTGAGGTATAGAGGT
TAAGAAAATTGCTGAGATCAGCAGCTAGGAAGTGGTAGAGCTGGATGGGACCCTGGCACCATGGCTCCCAGGTCCACAGTTGGTGGGACCTGGGCAA
CAGGAAGGAGACAGGGTTTGGTGGAAAGAGGGAAAACAGCTCATCATGACTGAGGCTCAGGAATTGTTCTCAGAGGACATCACACTGGGATTCAGAC
AAACTCAGGGCTTGAATTCTGGCTCAGCCACTTCCTTGCATAAGTCGCCCAACTACTCCATGCCTCAGTTTCCTCATCTGCAAAATGGGGATAACAA
TGCCCACCTCCCAGAGTTGTTGTGAGGATTCAGTGAGATTATGTATTTGAAGTGACTGGCACAGAGCAAGTGCTACATAAATAGTTGTCATTAGTAT
TGCCAGTATCCGGGTACTGTGGGGACAGAAGGAAATCCCTGTGGGGCAGAGGAGAGACTGCGTCTGCACAGCAGGAAGGGGCTCAGTGGCACCTTCT
TCCTGGAGATGTCTGGGAAGCACTCCGACCTTTGAGGCCAATCAAGCCATGCAGGCAGTGTGCCAGAGCTCAGGTGCTCTGCTGGGCTGCGAAGACC
CACCTGGGACCCTGAGGGAGGTAACTGGTGAAACCAGGAAGCAGGGAGCCCCTTGGAGAGCTGGGTGAGGAGCCGCCTTGCCAGCATGGGCCCCCCG
ACTCCTCAGTCCCCCAAGCCAGCCGGCCTGGCTTTCTCTGTGGATATGGCTGTGTGACCTCGCACAAGTTGGTCCTCTTTCACGGCCTCTGCTTTTT
CTTTTTCCTTTTTTGTTTTGAGACGAAGTCTCGCTCTGTCGCCCAAGCTGGAGTGCAGCGGCATGATCTTGGCTCACAACAACCTCCACCTCCCAGG
TTCAAGCGATTCTCCTGCCTCAGCCTTCTGGGTAGCTCGGATTACAGGCGTGAGCCACCTGGCATGGTCGGCCTCTGTTTTTCATCTGTATAATGGG
CTTTGTGCTCCCTTAGGGCTCCTCCTGTGGGTTCTGCTGCTGGACCAGGGCTCAGCCTCCAGTTTAGCTTGAGGGTGCCCAGGCCTGCAGAAGGTGT
CAAGCAAGGGCTGGGAGAGGGGGAGGAAGGAGGAGGCAGGAAGGCAACGGGAGGGGAAGGAGGAGGGCAGAGGAAGGGTCTCCGAGTGCCTCTCACT
CCCGCCTCAGCTCTCCGCTCCAGTGGTCCCATATAGGGCTCTGGGTCCCAAACACCAGAGGAAGGAGCGGGGACAGCCCAGGCTTCTTCCCTGGGTC
TTGTTCCTGAGCTGCCCATCTAGGATGGTGCAAGGCCGAGTTGTCTGCACTGAACTCTCCCTCAGTTTCCCCTCCAGTGATTGCAGGGCTGTGGGGG
CAGCTGGCACAGCAGGGCTGGGCTGGAGATGCTGCTTAAGAGCACGGGAGGGGCAGCCTGAGCCCAGAGGGAGGCTCAACGGGGACCTTTGCAGCCT
GGCAGACAGGGCAGGGTACTTAGCTCCCTGGCCTCAGTTTCCTTATCTTGGAAATGGGACAATGGCGATGATAATGAAGTTTTTATTACAGACCTGC
CGTGAGGATTACATAAGGTAACAGACACAAGCACTCAGAACTGTGTCCAGCACATGATAGGCCCATAATAAACCACAGGGATTATATGATCACTCTA
ACCTGCTTCTAACAGGGCAGGGGCAGTGCTGTGTGCGCCGTGTGCCCATCAGGGCTCTGGGCTCACTGACTCTACCCACTTGGCATGTGGCTCAGTA
TGGAGGCCCAGGGTGCAGATGGGCCAGGCCTGAAGCCACCTTCACCCCATGAAGACCCAGGCAGGCACTGCCCAGCAGGCAGGGTCACATAGTGATG
ACCCCCATGGGCTCTGGAGTCTGACACCCTGGGTTCCAACCAGCAATTCCATAGGAGCTCTGTGGTCTGGGGTGCTTCATCAACCTGCAAGGCTAAG
CCATCTGTAAAATGGGGCTAATAATGACCCTACTTTGTGCTGGGGCTATTTTGAGGATTAAATGAGGCAAACCCAAGGAAAAGCCTCTGCTCAGGGC
CAGGCACCGGGGAAATCTAGTAATTAGCCATTACCACCGATAAATCAGAACTTCTGGGCCGGGCGTGGTGGCTCGCGCCTGTAATCCCAGCACTTGG
GAGGCCGAGGTGTGTGGATCACGAGGTCAGGAGATCGAGACCATCCTGGCTAACATGGTGAAACCCCGTCTCTACTAAAAATACAAAAAATTAGCCG
GGCGTGGTGGTGGGCACCTGTAGTCCCAGCTACTCGGGAGGCTGAGGCAGGAGAATGGTGTGAACCCAGGAGGCGGAGCTTGCAGTGAGCCGAGATC
ACGTCACTGCATTCCAGCCTGGGTGACACAGCGAGACTCTGTCTAAAAAAAAAAAAAAAAAAAAAAAGAACTTCTGGCTGCTGCTTCTTTCTCTTTA
GAAAAAGCTCAGTGCTTAAGACCTGGAATGCCTCACAGATGTTACCATCCAAACTCCCAGATCTGGCTCCTCAGTTCCTCCACCAAAATAAAACAAA
TGGAAAAAGGAGAACCTGCCTCTCCCCCTGACAACTGAGCCTGGAGACAACCAAGGTGGATGGGTAGCATGCCCTGCACGGTGCCCAGGGGAGCTCT
GTGCCTTGGAGTCTCCACGAATTGGCCAAGGCCAACATCAAGCTCAAGTATGGAGGAGGGACCCGGGGGAAGGCACATGGCACTCTCTGACCCACCA
CAGACCAAAAAAGCCGTAGGCAAGCTAATTCACTCTCTAAACCTCAGTTTCCTAGTCCAGGAGATAGTGATGTTCTTCATGTTTGGAATTGATCAGA
AATGTTGTATATAAAGTGCCTGGCAAGCAGTAACAGTTGCCAAATGGCAGATGTTTGGCTCGGCCCTGTGTCCAGCCCAGAAGCTTTTGTGTACTAA
AAAGCACACAAAAAACCTCACCCTGCCCCATGGGGTCTTCTCCCGGCCCCTCTTTCCGAGGCTTTTGCCATCACTTTTTTGTGGGAAACTTGACTTA
GGCCAATGCTGGACCAAAGCCTTGGTCAGTGCTGGACACAGAGGGAGAGGAGATGGGCCCCTTCTTCCAGCTCACACTGCCCCTGGTCATAGAGCAG
ATTGCAATTGTCCTTGAAGGCCACTGACACTCACGGATCATTTTCTCAGGTTCTCCCAGAAAATGTCATTTCTGCTGTTCCTGTGCTGAGGGCAGGG
AACGGAAAAGTCGTAATTCCTACCTTGGTTTTCTAGTATTTTAAACTCTTCTGCCCCACCCCCATAAGGTCATTCTTTCTGTTGTTGACTATAAAAC
CTGAGAGAGTTGATGGGATATTGATTACAAAGCGAGGGGCAGAGGAATGGAGTGAATTGACCAGTCACCTGGTGTTGGGTGTGGGCAGTGAGATCTG
AGATCTCCCTTCTGACTCTGTGCTAAACTTGTTTTGACACAAAGCCAAGCTCCAGTCTCTCATGAGAAAAGAGGTGGAGCTCTAGAAACTTCTAGGA
AACTGGAAAAGATGGTCATGGAGGCCCTGGTAGACAAAGGACTTGGTGCAAGCCAGCAATTCCTTGGGGAGATACTGGGAGGCTCTGGGCTGGCTGG
CACCATCGCAGGGAAGAGGCTGAGGGCATTGCCAAGGGTAGTGTGTGTGTGTGCGCGCGCGCGCGTGTCCCATGAGTCACTGGAAGTGTCCATGTGG
CCCCAGTATCCTGGACTGCCTTGGAAATTGCCCAGCCTGATCTCTTCAATGCCCATCAGGGAAGCTGGACCCAGAACCTGGAAAGGACTGGATCTGG
GCCCCAAATACCATCTTTCTAGTCCAAACCTCTTTTCATGGCTTTGCATTGCCCCCAAGGGCCCCCACAAGCGGCTTCTGCGGTCCCATCCCCCTGC
AAGCTGGATGCAGCCAGAGGGCTGACTTGCGTGGGTCAGAGCTCTGCGCTTTGAGGGGCGGCATGCTCCCGAGGTCAAAGGTAGCACATTCAGGTGC
CAGGGCACAGGCCCTCTGCCTCTCCACCTACCCCGGAGGGTTCACAAAGCGGCCATGGTGCCCAGGATGGGCTCTGTGCTTTGTGCCCACAGTGTTA
CTACCCTCTGTCTGCCTGCAGGTCCCTGGCCCTGGGCCAGCAGTACACATCTCTGGGCTCACAGCCCCTGCTCTGCGGCTCCATCCCAGGCCTGGTC
CCCAAGCAACTGCGCTTCTGCCGCAATTACATCGAGATCATGCCCAGCGTGGCCGAGGGCGTGAAGCTGGGCATCCAGGAGTGCCAGCACCAGTTCC
GGGGCCGCCGCTGGAACTGCACCACCATAGATGACAGCCTGGCCATCTTTGGGCCCGTCCTCGACAAAGGTACTGCCTTGGGGCTGAGGGGGGGGGA
GGGGCTGGGTGGGGGGAGGGATCAGGACTGTATGCCCTTCCACCCCAGTCAGGGTGCCTCTTCTCTGAAGCCAGGGTCCCTCTGGGAGGGTGATAAA
TTTGGTTGCATGACTGCCACACCTTCCCAGGCGCTGCTGGGGGCTGGGCCGCCTTGAGATGCTGCAGGATCCTGAGGTTTGAGGGCCGACACCAAGA
AGGCCACAGACTTCCTTCCTGGCTTTTGCACAGTGGGCGCCTCAGGCTCCTGCTGTGGACATTTGTGACATTTCCCCAATGCTGTGTTAAGTGCTAT
GCAAATACCGCTTTGGGGTGGGGGTGAGAGCATTGCCACCCCTAGGTCCCTCCCATCTAGAAAGTCCTCAGTAGGTGAGGAAGCTGCCTGGTGCTGT
GGTAGCATTGCGTTGCCCCCAAGAGCCCCCTCTGGGTTCTGTTCATCCTCATGCCCTGTCAGCTGGATATGCCCAGGAATCATGGTGGGGCTAGAGG
GTGCACTGGCCTCTGGGTAACTTTCCGGCCGTCTTGCAGCCTGGGAGTCTAACTGGCTGTGCCTCAAGTGGACAGGGAACCACATGATTTGACTTTG
AACCTGCAGGCCCTGGGAGGGAGGCTAAGTGGGACAACTCCTCCCAAGAAGGGGACTTGGGGGGCTCATGGATGGGTGGAAGATGGGCAGCTCTTTG
TGGAACGGCAGTGTCCAGGAATTTAACGATCCTGGCCGGGGTGGCCACAGCCAGAGTTGCTCTGCCACCCAAACCGCTGAGCCTGGCCCTCCCTCCT
ATTAGAAGCTAGGGAATGAATGAGAGCTGCCTATACAGCCAGAAAGAAAAGGGATCTGACGTTCAGGTGAAGGGGTGGGGTGCTGGGTGGGGTTGGG
GGGGAAGCTGACAACCCCCATTTTAGAGTGCAGTTCCCCTTTAGCGGTGCCTCAGGTGCTCAGGGTCTGTCCCACCTACAGGCAAAGTGCTGGCTAT
TGTCATGGGACAGGAGGGTAATTAGCAAATCAATGGATGATGATTCTGTTTACGCTTCTATTGCCAAAACTAATTATTCCTTGTTGACATAAAAGAT
TTGGCCTCTTAATTGGGGCCAAGTGGCGGCGGTTTGCATTTCAAACTCGTTCTTGTCGGTGTGTTGGTCTCAGCGCTTTCCGGTGCCCCTCCTCGGG
CCAGGTAGCGCTGAATCAAACAACCCTGCGTCTAGACGCGCTCCCCAAAAGCGAGTCACCTCTGATCGCTCTTCCCGTCTCCGGCCAAGAGCCAGGG
GAAAGCGGCTCTGGGAGCGCTACCCATTCACTCCGCGTTGAACGTTTTAAGTCACCACCGTAGGTTCTGGCACACGCCCCGGTCCCTAGATGGCCCG
AGGACAGGTTTGGGTTGCGGGGGGAGGGGCGATGTGTGCCCGAGAGTTTGGGAATTTGCTGGCGGCGGCGAGGAGAGCATCCCGGAAGACGCCGCGA
GCGCATACGCGCCCCGGTCGGTGCCCAGAGCCCGGACCTTGAACCCAGGTTCGCGCCCAGGTCCGGCCCCACGTTTGCACCCCGCCCCCGGCGAGTC
CCCACACCACCGCCGCGTGGGCGGCCAAGGGTTAATCTCTGCCCCGCGCTTTGAGACCCGGGGCGCAGCGGGTCGGGCAGCCCGGGGCGCGCGTCTT
CCGCGAGTCTGGGGGCGGCGGGGTCGGCGGCCGGAAGGGGCGAGGCGCGGCGGCGGCGCGGCGGGCCCGAGGCGGTTCCCCAGGCGGGGGCGGGGGC
AGGGGCGGGGGCGGGCGCCGGAGGGGCCTGGGCGGCCTGAAAAGCGCCCGAGCACCGCCCCCCCACCGCGCGGCCGCCGCGCCATTCACACGCCCTC
TGGCCATTCGGCGCGGCGCGGCGCGGCGCGGGGGCGGGGGGCGCCGCTTCAATGGGGATTTCGCGGCCCGGCGCCGGGGCCGGGGGCGCGGCCCGGC
CGCGGGAGCCGCCCGTTGCTACGCGGTGGCGGCCGGCCCGGCGGCCCGGGCCCGGCGGCCGCATTATGCGGGTAATGCGGTGTGACACCGCGCGAAC
AAAGTCGGATTGACCGGCCCAGCGGGCCCCGCCGGACGGGGAAGGCACAGCGGGGCTGTCAGCGCCGCTCCCAGGCTAATCCCCGCCCCGCCCCCGC
CGCCCCCGCGTTCCCGGGGCTGGGAACCCAGCGCGGCCCGGGGCCCGGGCGCCGCTGCCGCCGCGAGAGGCCGCAGGCCGGCCGGAAACGAGGGGCG
CCCGCCCTGTGAACCCGCCCTCGGCCCCTCTTCCCCGAGGCGCCCCCAGGGCTGGGGCGTCTAGGATTGGGGCGAGTGAGCGGCGCGGCTGGGACCC
CGAGGCCGGGAGCACAACTGGACGCGGGACGTCGGGCCCCCTTGCCGCCGTCCTCGGTCGGCCCTGGCAGCGCAGGCGCGGGGCGCGGGCGGGACCA
GCTCTGCACTCTTCGCCCTCCCGTCGTTGGAAGTGGGACGGGCCCCTCTGTAGGTGGCTCAGGAGGGCGTGGGGGCCGCGGAGAGCGGCCGCGGGAG
CCAAGAGGGGTCCCGGGCTTCCTTCTTGCAGCACTCGGCCAACAGCCCAGGAGGGGAGGCGGGCGCCTGGCTTCTAAATAAATTTGGTCACATTTAT
GACCAATTTTGCCGACTAGGCCGGCCCGGCGTTTCACAGCCTGTGTCAGGTGGCGAGTCACCTCCGGCTCCCGGGTCCTGCCATGAGGAGAGAGATG
GTTGGGCTGCAGGTCTTGCAGGCCTTGGCCTCCCTAATCGTCAATGTCCACATCTATAAAATGAGTAAAACTTTCCATGTGAATGAAGCACATGCAG
AGACGATGACCGTTGTGAGGAGGCTGGAGCACACCCTTGAGAGTCTCTCCTTTCCCTGCGCTGGACCCTGGGACAGAACCAGGGGAACTGCCCGGTG
TCCCTACCCTTCTGGGAGGGAGTTGGCCGTGCCTGGGCGTGAGGGCAGGAGAATGGGGATGGGGAGACTGAGGCACGGGCCACTGCCCTCCTGGTGA
GGGGCTGTAGGAGCGGAGGGAAGCCGTGTCCATTTTACAAAAAGGGCGACTGAGACCGAGAAAAGGAGAGGGGCCGTCCCTATGCATCCACGTGCGG
AAGGGGTGGGCCTGGGCAGGTTTAGGCTGTAGATTCCCAGGCCTCCCTCCTTACAGACCTGGATCTCACTCAGGCGTGGGAGGGCTCAGGCCTGTGC
AGGGTGGTCTCTGGACTTTGGGAAGGTGGAGGGCAGGGGGTGTGGGACCAGGACAGAAGTGGCTGCCTTATTTCTGCAGCAACCTGCCAGAGCTTTG
GTGTCGGCGCCTGTTTCTAACAGGCACTGCTTTGCCCTGTGGCATTTGAGGTCCTGAGCTAATCAGCAGCCCAGGGCCTCAGTTTGCTCATCGGTGA
AGTGGGAATACCCAACTGGGTCTAAACCAGCCAAATAGGGTGGGGAAGCTGCCTCCCTCTTGCCTCAGGGCTGGTGAGCTCTTCAACTCTTCCTGGC
CTCGTTCCCCTCACGTCCTGGCCTGGGAGGTGGAGGCAGGGCAGGCGCTCCCAGGCCGGGTCCTGAGTGCTGCCTCCCCACGACCTCCCCGCAGCCA
CCCGCGAGTCGGCCTTCGTTCACGCCATCGCCTCGGCCGGCGTGGCCTTCGCCGTCACCCGCTCCTGCGCCGAGGGCACCTCCACCATTTGCGGCTG
TGACTCGCATCATAAGGGGCCGCCTGGCGAAGGCTGGAAGTGGGGCGGCTGCAGCGAGGACGCTGACTTCGGCGTGTTAGTGTCCAGGGAGTTCGCG
GATGCGCGCGAGAACAGGCCGGACGCGCGCTCGGCCATGAACAAGCACAACAACGAGGCGGGCCGCACGGTGAGCCCGGCTACCCTCCCCAAACCCT
TCAGGGAGCAGCCCCCCGCTTCCCCTCCGGAGCTGCCCTGGCCCCCACCTCACCCCTTCCGGCAGCCCTGTGGGTCGCGGGCTCCTTTTCCCCTTGG
CCAGGCTTGGCCTCCTCCCCACCCCACAGCCACTTCGTCTGTCCACCCTCCTTCCTCCTTGGGCAGTTCCTGGACTCCACCCCACCCGGGATGAGTC
GGCCTGGGAGCATGGCCACCCCGTGGGATCAGGTGCCACCCACCACCCATCCTGTGCACCTTGGCACGCGGTGTAGGGCTGTCAAGTCCTGGGAACC
TGGCTTTGAATTCAGCCTCCACTCCCTTACTATGCTTGTATGATCTAGGGTGAGTCACGTTAAGTTCCCAGCCTCAGTTTCCCCATGTGTTAAATGG
AGATAACTCCTATTTCTTTTTTCTTTTTCTTTTCTTTTTTTTTTTTTTTTTTGAGACGGAGTCTCGCTCAGTCGCCCAGGCTGGAGTGCAGTTCTGG
ATCTTGGGTCCCTGCAACGTCCGCCTCCCGAGTTCAAGCAATTCTCCGGCCTCAACCTCCCAAGTAGCTGGGATTACAGGCGTCCGCCACCACACCA
GCTAATTTTTGTATTTTTAGTAGAGACGGGGTTTCGCCATGTTGGCCAGGCTGGTCTCAAACTCCTGACTTCAGGTGATCCTCCCGCCTCATCGTCC
CAAGGTGCTGGGATTACCACCGCGCCTGGGCAACAACTCGTATTTCATGCGCTGTGAGAAGTGGACGTGCTGTCTACCCATGCTTGGGGTGTGAAGG
GGGAGGGAAACATGGTCACTATTCTTTAGGTCTTGCCTCAGTCCCCATCAGTTCTCTTCCTGTCACAGAGGAGTAGAGAAGGCTGGAGGGAAGGGGG
TGGGCAGTGGCAGAGAGGGAAGCCTGGATGTGCCCCCTCGTGGGGGGTCGGTCTGTTGGCCAGCTGCTCTTCTCTCCCCCAGACTATCCTGGACCAC
ATGCACCTCAAATGCAAGTGCCACGGGCTGTCGGGCAGCTGTGAGGTGAAGACCTGCTGGTGGGCGCAGCCTGACTTCCGTGCCATCGGTGACTTCC
TCAAGGACAAGTATGACAGCGCCTCGGAGATGGTAGTAGAGAAGCACCGTGAGTCCCGAGGCTGGGGTGGAGACCCTCCGGGCAAGTACTCGCTCTT
CAAGCCACCCACGGAGAGGGACCTGGTCTACTACGAGAACTCCCCCAACTTTTGTGAGCCCAACCAGAGACGGGTTCCCTTTGGCACAAGGGACCGG
ACTTGCAATGTCACCTCCCACGGCATCGATGGCTGCGATCTGCTCTGCTGTGGCCGGGGCCACAACACGAGGACGGAGAAGCGGAAGGAAAAATGCC
ACTGCATCTTCCACTGGTGCTGCTACGTCAGGTGCCAGGAGTGTATTCGCATCTACGACGTGCACACCTGCAAGTAGGGCACCAGGTAGGGCTCCGG
GAAGCAGGGGGGAGGCTGGGAGCCTGGGCGCAGGGAATGGGGTTGTTTGCCCATCTCGTCTTCTTGACGACCCCCTTCTGTTTCTAAGCTATCCAAG
ACACACAAGTTCCCACAGCCAAAATAGGAAGCTAGGATTTTTCAAGCATCCGTGGGAATGGTCCTTGGGCAGAGACTTGGTGGATTGCACAAAGCAC
AGATAAAAACCCAGTGTTGGCCGGGCGCGGTGGCTCACGCCTGTAATCCCAGCACTTTGGGAGGCCGAGGTGGACGGATCACCTGAGGTCAGGAGTT
CGAGACCGGCCTGGCCAACATGGTGAAACCCGTCTCTACTAAAACTACAAAAAATTAGCCGGGCGTTGTGGCAGGTGCCTGTAATCCAGCTGCTTGG
GAGGCTGAGGCAGGAGGATTGCTTGAACCTGGGCGACAGAGGTTGCAATGAACTGAGATTGCACCACTGGACTCCATCCAGCCTGGGCGACAGAGTG
AGACTCCATCTCAAACAAACAAACAAACAACAAAAAAAAAACAAAATTCAGTGTGTGCACATTAGGTGCCAGCAATTGATTAACATTTTTACACAAG
TTAGTTATGAGCCAGATCAATTTTTTGCATAGCTGCTAGCTGTCATTCTTGAGCATTATTTGGAGATGGGGGGCTGGAAGCTGGGAGACCTGACGTC
TGGTCCTAACTTTGTCAGTAACTTCCTGTTCTTTCACTTTTCTGGGCCTCAGCTTTCTTACTATAAAGTGGTGATGGTGGTGAAGGGCAGGGGGCAG
TTTAAATACCTCAAGCAGCGAGCCAAAATGTCTCCTCTACCCCTGAGGCAGAGTCAGTGTGCATCCACCTTGAACTTAGAGGGCAAGTTCCTGCACA
AGCATAAATCGTACCAACCTCAGGGAGGTGGGTGGGGGGAAGCGGCATATAAGCCCATATCCTGACAGATCTGAAGCTTGGGTGGAGTGGGGAGCAC
TCTCACCGAGTTTATGGATGATGACGGGGAGCAGGAGGGATTCAAGTTCAGAGTGGCTGGGATGAGTAATCGAGGGATCAAAGGCGGGTGGATTTTG
TTCCCAAGCTTCCCAGAGGTGGCCTGCATGGGAGGCGCTATGCACTGGCCCTGGGTTCCACAGCACCCCGGGATAGGTCAGCATGGATGAGGAGTGG
TCCTGGACCCCCGGGGTGGAAGGTTTTAAGGCTGGGGTGCCCTTTCTTCCTTTTCTGTCCCCTGAGAAGGAGCCTAGTGTGTCGAGGCATGAGCATG
GGTGTTGGAATTAGGCCCTGCCCTGCTCCAGCTCCTACCACTTGCCAACCGTGTGACCCAAGCAAGAAATTCTTCATAGACCTGGTTCCTCATCTGT
CATCCTCCCCAGAGATGCTGTGAGGGACAAATGAAAGGACACCGAGGACAGGGCTTGGTTAATTGGGAAGCACTTCCCTGGTGGGTCATTGTTGTCT
GGGGTGCTGCTGGGCGAGAGCAGCCTGTGAAAGGGTAGAGTGCCCCCTCTTCCTGCAGTTCCACAGGGCACCTCTGAGCTACTGGCACATGTCTGTG
CCATAGGGAGTAGGGGAGGGAAGTGGACGGGCCTCTCTGAGATATGGGAGGTACTTGACGTCATGGAGAAGAGCTCTGGCTGAGGGGCGACTCACTC
AGCCCATCTAGGAAGCCCCTCGGTGTAGCTCAAGTTCTGTCCTGGTGGGGACAGTCCAGAAAGGCAGCAGGTGGCACTACCTTTGTCGGTATCTGAA
CAAAAGCAGTGTGGGGAGTGGGAGAGGACGAAGGTGAATCCAGCGCCTCCACTCGTCCACTCAGCTGTCTGGCCTGGGAGCGCCAGTTTCCTCCTCT
GTTCAGTGGTGGTGACAATGGGAGCCCAGCAGAACTTTTTTTTTTTTTTTGAGACTGAGTCTCTCTTTGTCGCCCAGGCTGGAGTGAAGTGCGCAAT
CTCGGCTCACTGTAACCTCCGCCACCTGGGTTCAAGCGATTCTCCTGCCTCAGCCTCCTGAGTAGCTGGGATTATAGGTGCCCGCCACCACGATCGG
CTAATTTTTTTATTTTTAGTAGAGACGGGTTTCGCCATGCTGGCCAGGCTGGTCTTGAACTCCTGACCTCAAGTGATCCACCCGTCTTGGCCCCTCA
AAGTGCTGGGATTACAGGCGTGAGCCACCGCGCCCGGCCAGAATCAGTACTCTTCATCAGCCCTCTTTCTCCCCATTTCTGAAATGGACAATGAGTG
TTTTTTTTCCTCAATCTTATGTAAAGTGCTACATAGATATGGTTTCAAGATGGTGTGTCCAAGTGGTTCCTACCCGGAACCCCTTCTCTGTGGAAGC
TCTGGAGGCCTCACTCTGTGACCAGTGGAGGACACTCTCCCAACATGGTGCCCTTCTGATAAACACTCCCAGGCCAGCTCTTCTGCCTCGCTGAGCC
CTGGCTTCTTCATTTGTGAACTGGGGATGAGAACTGTGTTCTGTGTAGGGTTGTCCTGTGGACTGGAGACCCTGCACATGAAGGGCCTGGCCCAGGA
CCCAGCTGCTCACAGGGCAGCCACAGAGGCTCCTGGGCCTAGCAGAGCTGACCTCCCAGAGCCACCTCCCCAGGCGAGAGCCGCCTGCTCTGGAGCA
ATGGGCCCAGGCTCGGCCGAGACTCCGCTAGGCAAGACGGGAGCCTGCGGGATGCACTGGCAGGGATGGCAGGGGACAGAGTCGTTAGCTGTTCTCT
TGTCTTCCCTTACAGTCTTGGCAGTGCAGGCAGGAGAGAGAAGGGAGTCGCAGAGCAGGCCCCGGGCCCTCCACACGGTGAGGAGAGAGCGCCTGTC
AAATGGCTTGTGTCCTTCCTTTGTGGGGAAAACACCTTTTCTGTAGCTTTTTGTAGCTTTTACCCAGTGTCTCTGGCTAACCATTGATTTGGTGCCT
TACAAGTGCCAGATTTATTCCTCGTCACTCTGCTGTTGTGAACATGGTGAGCTGGAGCTGGAAAGGTTCTGGCAAGAGTCGAGTGGCCTGGCTGCGC
CCAAGTCCTGGATCTGTTACTGCCTTGCTGAGAGACCTTGGATGTGTCCCTTCAATCTGGCTGAGCCTGTTTCTGCCTCTGTAAAGTAGTCTTATCT
TGGCCTTGTCAGCCCCCTAGAAGACCTGGGGGTTGACTGGGAATGCCTCAAACCCTTTGTGAGATATGACTATGCAAATGAGTAAAATGAACACACT
GCTGCTCCAATGGGAAGGAATTGTTCGTTATTAAGTAGGCTCTCTCCCATTTGGACAGCTCATTATCAAGCTATTTGGTGCGGTACAAAAACACAGT
GCATGCCTGGCGTGAGCCCAGCACAGCAGTTGACTTGGAGTTGGGGACTGGGCCCTCGGCCAGGCACCGTTTCTCCCATCCTGAGTGACCTGCTCCT
GGGCTTCCTGTTGCCCAAGGCCCTCAGCTAGAGGTGGGGGAGGTAGAATTTCGATCCCATTCTGTGGACTCGAAAGCCTTACTGCTCCTGTGACTCA
TACGACCTCCCTCAACAGCAGGCCCAAAGCACAGAGGAGAGCGTGGAGAAACCACCCAAGTCCTCATCACCTCTGAGCACCAGGGACACGTTCAGAT
CCATCAGCAGCCTCTGATCTTTGAACCCCAATGGGTGGTCTCTGGGAATGGAATTATGTTTCTAATTCTTCCTCTTGATTTTCCCCAAGGGCGCTGG
GAAGGGGTGAAGTGTGTGGCTGGGCGGATTCAGCGAAGTCTCATGGGAAGCAGGACCTAGAGCCGGGCACAGCCCTCAGCGTCAGACAGCAAGGAAC
TGTCACCAGCCGCACGCGTGGTAAATGACCCAGACCCAACTCGCCTGTGGACGGGGAGGCTCTCCCTCTCTCTCATCTTACATTTCTCACCCTACTC
TGGATGGTGTGTGGTTTTTAAAGAAGGGGGCTTTCTTTTTAGTTCTCTAGGGTCTGATAGGAACAGACCTGAGGCTTATCTTTGCACATGTTAAAGA
AAATAAAAATGAAAAAAAATTTGACTCCAACAGAACAGGCTGGGCTAATGTGAGCTCTCAGCCTGGCAGTCAAGACATCAGCATGGGCAAGGTTCTG
TTTCCAAACTGCTGCTTCTGGTGACATTCCAAGACGCCTGGAGGGTGGGAGTCAGGAAGTAGGACACACCCCTGCAGTCTCCTTTTCTTGGTCCACT
CCCATTCAAATTTGAGCTAATTTCTCATTCTGATAAAAGCCATAGGTTTAGCTAGGATGAAGTGGTAGGAAGGTCCGTGGCAGTTGTTAGAGTAGGA
TTTGGAGTTTGGAAGAACTGGCAGCTCAGGGTGGCCTGGTCAGCCGTTTGAAGAGCAGCCATGTGTTCTTCTCAGTCTCATTTTCTCTATAACCCTG
TTCTGCACGAGGGGCAGTCAGATCTCAAAATCTTTTTCTACCATTCTGCAGTTTCCACCGTCAATGCAGTTTTTTTTTTGTTTTTTTGTTTTTTTTT
TTTTTTTGGTGGTAGTGGACCTTGTAAATAGGCTATGTAAGGGGGCAAGTCTTCTCTAGCTCAAATGGCTTCCTAAATAAATAAGCGGTATCTTCAG
AAGGGGCCATTCAGTCCTTCCCAGCCCTGCTCACCTGCAGATTCTCTGTACAAATAACTCCAGGTAGAGCAGTTGGACTCCAGGTCACCTTAGTATA
AGTTAGACAAAGGGTCCGTGAGGGAGTAGCCATCAATTCCTGAAATTCCAACTTTGTGACTAGCAGATGGGGAGGATGAAAACCATCCCTTTGCTTC
CTCTCCAATACGGACCCATCTTACTGTGTCCTTTCCTCTCTGGGGCCAATGTGAGTAAACACAGACACAGAGTTCTTTCCCCCAGCTCTTCCTCCCT
CACCTGCATGCTGAGATAGCTTCCATCCATGCAGTTCCCAAGGATCTGGATTAGAAGTTCAAAGGGGAACCAGCAGTCACCTACTCCCTTAGGTGAA
GCATCVCACGGCTGAGTTCTCCCTGAGGCATACTGGTCCAGCTGAGCGTCCTAGAGAAAGCTAGCAATAGGGAGGCACATGGATTTCACAGTATGAA
TTGGTTCAACAACTGTCTTAGGGAGAATCAGAAAGAAGAGATGCAGCAGGGGAATGAGCAGAACAAAGATTTTTCTTTCTCCCCCTTCTCTCTGGGG
TCTACCTAACCCTGACCTAAAATACCAGGGCAGCGATCTCCCAGCTGGTGCAGGTGGGCTTGCCAAGATGGTCGTCCAGGAGCCCGCCTTCACTTCT
AAATCTGCTGGCCACAAGCCCTGCTAAAGATACACATCTCACCCCCTCCGCCAAGTCTGAAATGCCCCTCCCCATCTCACCTTAGACTGAAAAGTTT
TAAATCATGTCAACTGGATAATACTTGCTTTATGTGAGAATACTTCAGCAGAATGGATACGAATTTTCAAAACAATCTTTTCATATCTATGTATTCT
ATATTAAAAAGTGATAAAGTCATGTTTCTGGGGCGTATTCAAGTAGCTGACAAGTAATTATTAATAATAGTACATGAGTGCATTGTAATGATTCTCG
CCGTAGTCAGGTAATAGTATCCAAGCGAAATTTCCTACCAACCTGCTGTATCCAAAGTTTTGTAAAAAGTTGTAGAAGTTGTTGATCTTTTTGATTT
TATATTCAAAAAGTCTCTTTTTATAAATATTATTTATTATACAATGTATATACCTTTGAGTTAACTAAGATTATATATTATATAAATATATATATAT
TTGGAGAAAAATATATTTCATCATGCAGTTTTTTTCTGTTAAGTCATTAAAGAGAAGGTAAACAACCTAAAATGGATACATTGTACACTGTGTGGAG
TTTCCAGATTAATGCTCAGGAGGTCAAGGATACGAGATTCTGAACATGGTGGCCTCAGCTAATTAAATGACCAAGTGAAGGTTATTTCCTCTGTTTT
CTAAGCAAGATCATCAGAGATGTCACGGACAGTGGGTCTGATACACGACAGTACATTTTTAAAGGCTTCTTTTGTTCTAAGTTAACTTCTAACAATG
AATTTCCAGTTTTAGAAAACAACAACTTGTGTACTAATTCATTTCACAGACTCAAGTATTTTCAATGTATAAATTAATGTCACAGGCACTCCTAAAA
GATGTGGCCCAAGGCCAGCAGACCGCAGGGGTGGCCACTGCCTTAATCCCCTCCTCACCACGTTTTCAACTCCATTTAATGGGAAAATAGGATTTTC
TCTATTGAAAATGAAACTTGACTGAAAATTGTCTGTCCCCTGCTAGCTCCACTGTGAGAGCTAAGCCGTAGAGGGAGGTAGGCTGGTCTACAAGAAG
AAAGCGTGTGCCCTGGGCTGTGGGCTGTCTGGAACAATTGTCATTTGAAGTTGGGTTTGGGTTTGGGTAGAAAGGGCCTGCCAGTGGGCATGCTCTG
GGGATGGGCACCCTCTCAGATCGCCACTTCACAGGACCCACTGGAAGCTCACAGGTTATGCCTTCTCAACAATGAGATGCCACAAGTTGGTTTTATG
ACAACAGACAATCCTTTAGTTTCAGACCTACCCATAAAATGTCACCTTTGGTTTATTCAGCACATTATTTCAGAGAATAACAAAAATGTTTTTTTCT
TTTTATTTCTCCTGACCTACCTCATTTAGGAACAAATCTTACCTCACTACGAAGAGAAATAATGAGAACAGGCTGGGTGGGGTGGCTCAGACCTGTA
ATCCCAGCACTTTGGGAGGCCGAGGTGGGTGGATCACTTGAGGTCAGGAGTTCGAGACCAGCTTAGGCCAACATGGTGAAAGCCCGTTTTACTAAAA
ATACAAAAATTAGCAGCTGGGCATGGTGGTGCATGCCTGTAGTCCCAGCTACTTGAGAGGCTGAGGCAGGAGAATCACTTGAACCTGGGAGGCGGAG
GTGCAATGAGCTGAGATCGCACCACTGTACTCCAGCCTGGGTGACAAGGCGAGACTCCATCTCAAAAAAAAAAAAAAAACAAACAAAAAAAAACCAG
GAAAATAAAGCCATGAGCCGCCTATGGTGGAGGCGGGCTGCTCTGCGCTGTATTCAGACCTGTGACCTGAGGTTAGGAATAGCCACCAGAAGTTGAT
GCACTCTGTTTTGTCCATGATGTGTCTTTAGCCCCCTGAAAAGCTGAAACCACAAAAACTGTTACACTTCAGCCTACAACGAATCGGACACGTACAG
GATACAGATCTCCTCTACATCTTACAGTCTGGTCATTTTTATTTCATTTCGACTTTTTATTTTTCTTTTTGCATAAAGCTAATGCAGGTGACTACAC
CTTTTTCTGAAATGCTTGGGACCAGAAATATTTCAGACTTGGAAGATTTGCATATATACAATGAGGTATCTTGGGGATGGGACCCAAGTCTAAACAC
GAAATTCATTGATTTCATATGCACCTTACACACATAGCCTGAAGGTAATTTTATGAATTTTTAAATAATTTTTGTGCATGAAACAGTTTAGACTGCA
TTTTGAATTTTTGATTGCCACCTGTCACACAAGGTCAGGTGTGGAATTTCCACTTGTGGCATCCTGTCGGTGCTGAAAAGTTTTAGATTTTAGAGCA
TTTCAGATTTTGGATTTTTAGATTAGGATGCTCGACTGAACTTATCTATCCCAGAATTCTAGGTCTTTTTTTTTTTTTTTTTTTTTTTTTTTTTTTT
TTTTGAGATAGGGTCTTCCTCTGTTGCCCAGGCTGGAGTACAGTGGTGTGATCATGGCTCACTGCAGCCTTGACCTCCTGGGCTCAAGTGACCTCCT
ACCTCAGGCTCCCGTGTAGCTGGGACCATAGGCATGTGTCACCATGCCCAGCTAATTTTTTAATTTTTTTTTTTTAGAGATGAGGTCTTGCTATGTT
GCTCAGGCTGGTCTTGAACTCCTAGCCTCAAATGATCTCCCACCTCAGCCTCTCAGCATTGGGACTACAGGCATGAGCCACCCTGCCTGGCCATTAC
TTTTTCAATCAATAAGATTGCCAAATATTTAAATGGTCCCTATTCATACTAATGAAGACCATTTGTAATCGTATGATTAGTTTTTAATTTTTAAGAG
ACAAGTGTCTATGTCACTCAGGAGTACAGTGGCATGATCATGGCTCAACACAGCCTTGACCCCCTGGGCTCAAATGATCCTCCTCAGCCTCCCAAGT
AACTAGGACTCAGGCGCACTCTACTATGGTAATTAAAAATAAAATCTTTTGTAGAGATGGGGTCTTGCTGTGTTGCCCACTGGTCTCAAACTCCTGG
GTTCAGGGGGTCCTCCCACTTCAGCCTCCCAAAGTGTTGGGATTACTGGGTGAGCCATCACACCCTGCCCAGTCTTMTTTGATGGTTAAGGAGAATA
CAGTTGATAGCTGTGCTTGAACATGTAGTTTCTCCTTATGTATAAGTACTTACAAACGTCTAAACACAAGAGGACGTTGTTTAAAAATCTGAAAGAG
ACGACATAATTAGAAGATATCTTTCAAAGCAATATTATTTTATGAAGAAAGAAGGGAAGGAAAGCATGTGAGACAGTTCAAGGATGAGGGGATGAGG
TACAAAGAAAGGTTGGAAGGAAAGAAGGTAGGGAAAAAGAAAGCAAGGAAACATGATGGCGACATGATTCTTGGTGTTACCTAATGCCGTCTCCCCT
GGTGGCCCTGGGCATAGGGACATGTTCTTGTGTGTGTGGCTGGTCTGCACACTCGAGTGAAGTCAGTGCAGCTACGCATTGCTGCTACACTGCCACT
GTTTCTCCTAGTTTAATAGAGAACCTTCTAGGAGGCAAAAATTTCTTACCATTAATCTTATACTCCCAAAATAGTGAGACAGAAGTTGCATGGCGTC
TACGCTGGGAAAATTTCAAACATGATCCCTGCTTGGATTTTTTGTCTTCACCCACTTGGGATGCTTGCCAATCACAGGCGCTATCTCCCGCATCTTT
ATGCCATGGTGCCCGTCCAGCACACACCCTTGTGGGCTCGTCTCCAGGACCCAGTCTCAGTACGGCTTGCCAGCTGATGAGAAACTCTACATCATTT
TGCAATAAATTCTGCAACTTACCTGCTTTCTTGAACTCTCATACACACCCTTCCCCAGTCACTTTTTTTATTTTTCGAGACAAGAGTTTTGCTCGTT
GCCCAGGCTGGAGTGCAGTGGTGTGATCTTGGCTCACTGCAACCTCTGCCTCCCAGGTTCAAGCAATTCTCCTGCCTCATCCTCCCAAGTAGCTGAG
AGGTGATTGCCCGCCTCAGCCTCCCAAAGCTCTGGGATTACAAGTGTGAGCTACTGCGCCCGGCTGCCCCCAGTCACTTCTTTAAAGAGGCTATGGA
CCAAGTTGTCAGGCAAGGAAAATGAAATCCTGCTTTCACCTTCTGTGTTTGGGCTGGGGTGTTTGTTTATAGCTGACTCACTGTTTCAACAAGTCTG
TTGATCAGGATTTGATTATTGTACACACTTCAATCTACTCCATGATTTTGTGACTTGTTGCTGAAACTTTTAAAGGGGAGGCTGCAATTTTTAGGAT
ATGAAAAGACAGCCTTAAAGTAAAAACATATTTCTGACTTTAACCACAAATATGCAATTTTTCTGTGTGACATTGAAAAAAAGTGTCCCAGTGAAGA
TGTACAAATTTAGAAAGGATAAAATAACCTCCTCTTTTGAGGTTTTGATCTCAAAAACATCTTATGACAAAAAAATAGCCGGTTAACAATTCGCCTA
TAGATGAAGAAACCTAAGGCAGCAGCTAGCTGGAGCCTCCTTTGCTTCTTCACAGGCCGTTCTGTAATCTCATACGCTCTGCTGTACAGCACTGGCT
TTGAAGAATATTTGGGCAGCTTCAAGAACCCACCTTCTCAAGGCTATGCAGGGACCAGGTCGGCCTCAGGGTCCTCCTCCCAGGAACTCCCGTT
Human Sequence—mRNA
GCGCTTCTGACAAGCCCGAAAGTCATTTCCAATCTCAAGTGGACTTTGTTCCAACTATTGGGGGCGTCGCTCCCCCTCYTCATGGTCGCGGGCAAAC
TTCCTCCTCGGCGCCTCTTCTAATGGAGCCCCACCTGCTCGGGCTGCTCCTCGGCCTCCTGCTCGGTGGCACCAGGGTCCTCGCTGGCTACCCAATT
TGGTGGTCCCTGGCCCTGGGCCAGCAGTACACATCTCTGGGCTCACAGCCCCTGCTCTGCGGCTCCATCCCAGGCCTGGTCCCCAAGCAACTGCGCT
TCTGCCGCAATTACATCGAGATCATGCCCAGCGTGGCCGAGGGCGTGAAGCTGGGCATCCAGGAGTGCCAGGACCAGTTCCGGGGCCGCCGCTGGAA
CTGCACCACCATAGATGACAGCCTGGCCATCTTTGGGCCCGTCCTCGACAAAGCCACCCGCGAGTCGGCCTTCGTTCACGCCATCGCCTCGGCCGGC
GTGGCCTTCGCCGTCACCCGCTCCTGCGCCGAGGGCACCTCCACCATTTGCGGCTGTGACTCGCATCATAAGGGGCCGCCTGGCGAAGGCTGGAAGT
GGGGCGGCTGCAGCGAGGACGCTGACTTCGGCGTGTTAGTGTCCAGGGAGTTCGCGGATGCGCGCGAGAACAGGCCGGACGCGCGCTCGGCCATGAA
CAAGCACAACAACGAGGCGGGCCGCACGACTATCCTGGACCACATGCACCTCAAATGCAAGTGCCACGGGCTGTCGGGCAGCTGTGAGGTGAAGACC
TGCTGGTGGGCGCAGCCTGACTTCCGTGCCATCGGTGACTTCCTCAAGGACAAGTATGACAGCGCCTCGGAGATGGTAGTAGAGAAGCACCGTGAGT
CCCGAGGCTGGGTGGAGACCCTCCGGGCCAAGTACTCGCTCTTCAAGCCACCCACGGAGAGGGACCTGGTCTACTACGAGAACTCCCCCAACTTTTG
TGAGCCCAACCCAGAGACGGGTTCCTTTGGCACAAGGGACCGGACTTGCAATGTCACCTCCCACGGCATCGATGGCTGCGATCTGCTCTGCTGTGGC
CGGGGCCACAACACGAGGACGGAGAAGCGGAAGGAAAAATGCCACTGCATCTTCCACTGGTGCTGCTACGTCAGCTGCCAGGAGTGTATTCCCATCT
ACGACGTGCACACCTGCAAGTAGGGCACCAG
Human Sequence—Coding
ATGGAGCCCCACCTGCTCGGGCTGCTCCTCGGCCTCCTGCTCGGTGGCACCAGGGTCCTCGCTGGCTACCCAATTTGGTGGTCCCTGGCCCTGGGCC
AGCAGTACACATCTCTGGGCTCACAGCCCCTGCTCTGCGGCTCCATCCCAGGCCTGGTCCCCAAGCAACTGCGCTTCTGCCGCAATTACATCGAGAT
CATGCCCAGCGTGGCCGAGGGCGTGAAGCTGGGCATCCAGGAGTGCCAGCACCAGTTCCGGGGCCGCCGCTGGAACTGCACCACCATAGATGACAGC
CTGGCCATCTTTGGGCCCGTCCTCGACAAAGCCACCCGCGAGTCGGCCTTCGTTCACGCCATCGCCTCGGCCGGCGTGGCCTTCGCCGTCACCCGCT
TGACTTCGGCGTGTTAGTGTCCAGGGAGTTCGCGGATGCGCGCGAGAACAGGCCGGACGCGCGCTCGGCCATGAACAAGCACAACAACGAGGCGGGC
CGCACGACTATCCTGGACCACATGCACCTCAAATGCAAGTGCCACGGGCTGTCGGGCAGCTGTGAGGTGAAGACCTGCTGGTGGGCGCAGCCTGACT
TCCGTGCCATCGGTGACTTCCTCAAGGACAAGTATGACAGCGCCTCGGAGATGGTAGTAGAGAAGCACCGTGAGTCCCGAGGCTGGGTGGAGACCCT
CCGGGCCAAGTACTCGCTCTTCAAGCCACCCACGGAGAGGGACCTGGTCTACTACGAGAACTCCCCCAACTTTTGTGAGCCCAACCCAGAGACGGGT
TCCTTTGGCACAAGGGACCGGACTTGCAATGTCACCTCCCACGGCATCGATGGCTGCGATCTGCTCTGCTGTGGCCGGGGCCACAACACGAGGACGG
AGAAGCGGAAGGAAAAATGCCACTGCATCTTCCACTGGTGCTGCTACGTCAGCTGCCAGGAGTGTATTCGCATCTACGACGTGCACACCTGCAAGTA
G

TABLE VIII
MOUSE NOMENCLATURE
ICSGNM Batf
Celera mCG5472
HUMAN NOMENCLATURE
HGNC BATF
Celera hCG22346
MOUSE SEQUENCE—GENOMIC
CCCAGGCATGGTTTTAAGCGATTGGCTGGCATCACATGGTTACAGAGTGGCAAAGCCAGACACGAGCTTGCAAGGTCAAACTCACAGCTAAAAGACC
ATTTGCCTGTTCTCTTGCTTCCTAGGCCTTTTCTCCCCACCCCAACCCCCATCCCTATCATCATCTTAGGATCCTGTGAAGGCCAGAGGCTGCAAAC
CAATTTGCTTTACATTACACTCCCAAAGCACCTCACTCCTGGGAGGTCAGACACCTTTTGCAAGGCCCGGTGTAGACGGGATTCACCCTCCCTGCCA
AGAATAAAGGGCTGGGTGTGTCCCATGATTGCCTGGTTGTAGCACAAACATAGCTTTAGACATGCTGCTTCACTGTTAATCCATTTTCTTTTTATTA
TTGTAGCCTCTTTCACTTTTCTCTGTGGTCTGCACATGGATGTGAGCTCACTCACGTAGGCATGGGGGCCAGAAGTCAACAGAGCTGTCTTCTTTTA
TTCCTCTCAACGTTATTTTTAAAATATATAATAGTTGTTCTTTGAGAATTTCATGCATACATACGATGAGTTTTGGTCATATTGGCTCCTCAGTCTT
CCTCGCAATTCCTTCTAGATCCACCCCTCACCTTCCTACACCCTTACACCTTGGTGCCTCCTCCTTCTTTTAATAACTCATAAAGTTCAATTTGTGC
TGCCCAAATACACACAATCACTAGAACAAGGTCAACCTACCAGATGCTCCAAGAAAGCTGAGTTTCCTTTTCCTCTGACACTTTTTGTCCCCTTTCC
TGCAACAGTCCTGAGTCTTGGAGGGTGTATGTGATGTAGGTGCCATTTGTACTGAACACTCCACAGCACTTGTTCTCTGTACACTGACCAGTTCACA
AGTGTTTGCATTAACCACCATCCGCTGTACAAAGAAACATCTCTGATGAGGGCCAAGAGCTTCATTAATCTATGGATACAAGGATGCAAACTGAAAG
AACTGTCCATTTATCAAATAATAGTAGTAGGCTCACTTCTGGAGCCTGTGAGTGCCTCCACCATGGGCTTTGCCCAGACTTACAGTACCAGGCAAGA
GTTTTATTCTTTGGAGGAAGTCTTAAATCTATTCTGAAAGTAGTTGGGTTAACTCCATAATGGTTGTGCCACTAATACTTAGGTATATTGTGCCATG
TTGGGCATTACTGTGGCTACCAGAATACACAGCTGGGTAANNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNTGAGTGCTCTTAACTGCTGAGCCATCTCTGTGGGCCCACTCCCACGTTT
TGGTTTTGGTTTTTTGAGACAGGTATCTCTTTGTGTTGTGCTGGTTGTCTTGGAACTCTATAGATCAGGCTGGCCTCGAACTCACAGCGATCCACCT
TCCTCTGCATCCTGAGTGCTGGCATTAAAGGCATTTGCCACCACTGCCTGCAAGTCCTTCACTTTTTTTTTTATCTGATGGAAGACTAAAATTTAGT
ACCTCTCTCTTGCCTTTTGAGGGGCCTGGGTCCATGCCTTCTGGTCTGAAAGGTAAAAGCAGATTGGAAGCTTCCAGAAGCAACTGAGTGCCTCAGG
AGCTCTGCTGTCAGGTTGAAGACTCTAAAGCGGCTCGGAGCAAGGACGTCATTGGCCTGGCTTTATCTCAAGGAGGAGGAGACAGAGGAGGCCACAG
GCTGAGACTGACGGATGGAGCAGAAGTTTAGGATCTAGGAGCTTCCTCCCTCAGAGCATCTGGTGATGACCAGAGATGTGTCAGATCTTCTCACCTG
GGGTTCTACTGAAGTCTGGTGAGTGGAGGACCAGGGCCTGGCCTGCTAGGCCAGAGACAGCCCCCACAACAAAGACTCCTGTGGCCAGAAGATCACA
AAAACTGCCCTTTTCCTTTTCTGCTAGCCTCACGAAGAGCCAGCTAATGATGGTTTCCTGCAGTCTTCATCTAGGCTGAAAAGGCAGGGGACCAGGC
TACCGGCCACTGCAAGGGACTTTCCTCACCATCACCTGGCCATCCCCTCTGGCCCCTTCCGTGGAGAGCTCTGCCCTCAGGTAAATGTTTTCTCAAC
TTGTTTGGGGAACTTCGTAAAAACTCCCCGCTAGCCATCTCATAAGCTGTGAAGGTGTGCCTTAATGTGCTTTTTTCCACTAAAAAGGTTTGTTTAA
AAAAAAAAAAACTAGAGCAGCCAGGGGATTTTTATTATTTTTTTTGTTCAAATAACTAAATAGCAGCCCATATGAAGACTTCAAAGTTAACACATCT
CAAGAGTATCCCTCAAAAGGGAGCTCAGAAAAAAAAAGCGGCAGAGTGGGGGGTTTAAATGTCGTGAATCAACTCATGGCTTAGACAGGAAAAGTTG
GTAACCTGGGCGGTCTGGTCTGCCTCAGGAAACAGCCAGACAAGGTTGGGCATCAGTGCATGGGAGCATGGAAGGAGCCTTAAACTTCTCCCGTAGC
CGGGAACCAGCAGCCTAGCCTCTGGGGAGCCGGGGTCATTTGCATGCGCTCTTAAGAGACTCTTGCTTATAATTCCCTAGTGTCATCTTTAGTAACT
AAAGGGGTTTCTAGGGAATGGGAGGCCAGTGCCCCGGGTATATGAACTAGCCTAGCCACCTGGTTCAGAAGCACCAGTGAGAGCCAAGTGGGAGTGT
AGGAGGCCTTGGTGCCTGGAAGTTGCTAGATAACTTGAGGCAGATGGTGAGAATGGATCACTGTCTACTTGAGACCTTTTAGGCAGAAGAGTTTGAG
GCTGTGGGTGCTGATGGGACCCAGTGAGTCAACTCTTGCATATCCCCAGAGAGTCAAAAGATAACAGAGGCCAGTTGGATCTCCTCCTCTTGCTGAG
ACACTAAGCTGGCCCCTGAGGTCACACACTGTCCCCTACTGGACAATGTGTCCAGAACTTCGCATCCAACAAAGGGCCTGATGAGTGACCAGCATTA
GGGGTCAAGCCAGCATTTCCTTCCTTCTCCCTGGCTTTGGGAAATGTCCCACCGCCTTNNNNNNNNNNNNNNNNNNNNNNNNNNNNNNNNNNNNNNN
NNNNNNNNNNNNNNNNGCACCTTCTCCACAGTCAGCCCAGGCCCACACCCACACTATGTTGTCCTGGGTTTCTTATGTGACTGGAGCCTCCCTGACC
TTGTCAATCACCAGGTAGAGGGAGCCCAAAGCCTGTGAACTGAGTTGATAGTTGGATGTTGCAGTGGAGCCTGTCTTTATGGAAACAGGGAAGAGCA
AAGATAGGGCACCTGGGCTGACATGAATATACACTGTTACACTTAAAAAGCAAAACCAAAGAGACCCTGTTTCGGATGACCTTAAGAAACCCCTGTG
CAGGGCTTGGAAAGAGGGCTCAGCAGTAAAGAGCACTCCTTGTTCCTGGTGAGCTCCCGGCATCCATATGATAGCTTACAACTGTCTGTAAATCCAG
TTTCGGGGGATCCAATGCCCTTTTGTGACTTCTGCAGGCATCAGGCATGCGTGTAGTGCACGTACACACGTGTAGGCAAAATAGCCGTACACATGAA
AAAATAAATCTAAAAATATTTTTAAAAGACAGAAACCATTTGCGTATGGTATATTCTGCTGATCTGAAACAAGGTTGATGACTAGGCATTCCAATGC
CTGCTATGGAAAGACTGAAATACGGGTTGTCAGTTTGACCTTAGCCCTTAGAGTTTCTTCCATTTTGTTCCTACCTTATCCATTCTCCTGAAAATCA
GCAACCAAATCACTACTGCCATGGCTCAGAGGAGCTGGAGTCTGCTCCGACCACTGAGGGCTCACCATGGGACAAATGGACAGACAAGTAGTGGTCC
CTGATTGTACATCATCTCTGTTGCTCATGACAACAGCTGCGTGGGGCTCTCTGCTTATCATATTCAGCTGGATCCCTGCACATCGGCTTTTAATTTC
ACAAGTCTTCATCATGTCCTCCACGTAGCAGAAAATGCTCTAGAGGATTGTACACTATAACTCGTTTGAGTCTTGGAATAGCCTAGAGGAATTTGGG
ACTTTCCCTTTTGATAATTAGCCAGATGTGGTGGAGAGGTGGGCCTCCAGTGTGGGCTAGCTCCATCATAGAAAGTATCACCAGGAGGGTCTGGCGT
TATGGCTCAGCTGGAATAAAGGTGCTTGCCACAAAGGCTGAGGACTTCAGGTGATCCACGTGGTAGAACGAGAGAATTAACTCCTCCCAAGCTGTCT
CCTGTGACCTCCACACCCACAGGGCACACATCACCTACACATACACACACTCATATATACATTAAGACACCATGACCAAATGTAACTCTGGGGGGAA
AGGGTTTGTTTGTTTCCTTTTACAGGTTGTAGTTCTTTAACCAGGGAGGTCAGGGCAGGAACTTAAGACAGGAGCCTGGAGGCAGGAGCTGAAGGAG
ATGCCATGGAGGGGGTGTTGCTTACTGGCTTGCTCTCCATGGCTTACTCAGCTTGCTTTCTTATAGAACCCCAGGGTGGGACCACCGACAGTAATCT
GGACCCTCCCACGTCAATTATCAGAGGAGAAGATGTACCCCAGGCTCGCCCACAGGCTAATCTGGTGGGAACTTTTTTTTTTCAGTTGAGGTTCCCT
CTTCCCAAATTACTCTATCTGATGTCAAGCTGACATAAAAGCAGCCAGCACAGTCAGGGTTATAAAGTCAGCAGAGGGGACAGCCAGGAGAAAACAC
GCTACTGCCTTTGACTCCCCCTTTAGCTGCATCTGATGGAATTCCCCCAGACATTAATTTGGGGAGTATGGTGGGCTTAAGAAATGTCCTCAAGTAT
TGTGTGTGTGTGGGGGGGGGGGGAGGTGTTTCTCACTATGTAGTCTTGACTGGCCTGAAGCTAGCTATGTAGACCAGTCTGGCCTTGAACTCATGAA
GATCTGGCTGCTTCTCTACCCCCACCTCCACCCCCAAGTACTGAGATGACAAGCACGTGCCACCACAACCAGCCAGTCTTCAAATTCTTTAATATTC
CTACCTTAAATAGGTGGGGGTCTTAGCCTGTGGTGGTATACCCCTTTAAGATCAGCACTTGCAGCTGGGCGGTGGTGGCGCACGCCTTTAATCCUTG
CACTTGGCAGAGGCAGGCGGATTTCTAAGTTCAAGGCCAGCCTGGACTACAAAGTGAGTTCCAGGAAAGCCAGGGCTGTACAGAGAAACCCTGTCTT
GGGAAAAAAAAAAAAAAAAGACCAGCACTTGGGAGGCAGAGGCAGGTAAATCTCTGTGGGTTCAAGGCCAAACTGGTCTTCATAGTGAGTCTGTCTC
AAAACAATAACAGAGTTTGGAGAGATGGCTCAGAAGTTTGGTTCCCAACATTGATGTGGTGGCTCACAGTCATCTGTAACGCCAGTCCCAGAGAATC
TGGCGCCCCTCTTCTGGCCTTGTGGGTATTGCACACACATGGAACACAGACACACATGCAAACAGAAACATCCTACACATGAAAATTAATTAATTAA
TTAATTAATTAATTTTTAAAAAGATTGAAAGCAATAACAAAAGAGGTGAAGTCTCTTTAGTCTCCCCTTAAGAGTGAGGTGTTAGTGCTTTGCCTGA
GATTAGCAAAGCAGAAGAGAAAGGGGACTGAGACATAAATGGCTCCCTCTATTAACAGCCATGTGGGGACTCCACTTAGAAGTGAAATCTCCTAGTT
CATCAGGACTTCAGATGACAANNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNCCCTGATATTTATTATTTTTAAACATAATTATTTTAAAGTAATTTGTTAATATTCC
ATTGAATGAGACCCCTCCTTCCATACAGAAGACATCATTTGTATTTCTCTTCTCACTGGTTCCTAGTACTTAAACACTGTACTGACTCGACATCATT
GAGTCAAAGCCACCACTGACTGCTGAGTGATAGACTTTACAGAGCTAAGAACTTTGTGCACATTATAACTCATAACTACTTTGGTTGTTGGACTATG
CTTTTCTTTCTATGAAAGCAGTGTCTCTGTCTTGCTTATACTTATGATTCTTGATTCTTGTTACAACACATAATAGGTGGGAAATCTGTCGGGTGAA
ACTAAATGCAATATAAATAAGATCATTATAATCCTTAAGGGGTTTTTAATATACTTAGGTTTAGAGAAGATGCAAGTCGTAACTTCTAGTTGTATGG
GAGCTTAGTCGGACCTTATTCAAACAGCCCTTTATTGGGTGCCTACTAGGTACCGGACATGGAACTGTAAACATGAGACAGTCTTCAGCTTGAACAG
GGGCCACATCTCACCACCTTCATCGCCACCTCTGTGATTCATGCTACCAGTATCTTTTCTTTTGTTTAGGACAATGAGATTACAATGTTAAACCCAA
CATTGTAATCCTAACTGGTCTTCCCTCATTCACTTTTCTCCCCACAGGAAAATCTCAACTGTGTCTAAAGGGATCCCAGGATACTCCAGTCAGAGCA
TGTAGTTCCCCTGCTCTCACACTACCAACGTCTGCCCACCTGGGGGAGGCAGCAGGCTTGACCTTACAAGTCTTCAACTGGGTCTGAGTCAAGAGGT
GGACTGTATCTTCAGCCCACTGAACATTGAGCGACTTTGTGACTTTCTTTGGTCAATAGGATGTGGTAGATGGGACTCTGAGCTAGACCTGATCATG
GATGTTGGCAGGTTCTGAGTTCTGCCTTTAGAATCCGGCACCTGCCAAGGGAACAACAGGCAGGATGGCTGGGAGGGTGGCGTGGGGTGGGCCAGGG
AGAACAGACCAGTCAGTTACCAACCAACTCAGCAGCTGAGAGGTCTGGATGAGATCAGAGCTACTGTGGCCTTCTGGAATCTTCTGAATGACCTACA
AACTCAGCCAAAAGCATGCCTGTTTAAAGCTCTCGTGAAGTCCTTGGTATGTTACACAGTGGAAACTTACTGATAAACTGTCTCGTTCAGGGGAAAA
AGTCAAGTGTTTTGCCTGGCACATGGTGGGTGCTCAGGGGACGCTTGATTAAATAAGAAATTCCCAAATGCAGTTTGCCTTCCAGTGAAGAAAATAA
GTTCTAAAACAATCAGCGGCTACGATAAGTGCTCTGAAACAAACAGGCAGTCGCAGTGGCTAGATCTGAGGATCTAGGGAACGGGGCACGGGCCTGT
GGTTGGTTCTCTGGTTACTTAGAATGAGGCAAAACTCCTCCCAATTGACTCCAAGAGCCTTGAGAGACGTCCCGGCTGATCCACGTACCAGAACTTA
TCCCCTACCAGTCCCCTGCCACTCTCAGGACCTCTGATCCGGAGACACTGGGCTCTCTGGTGTTCCACCTCAGGTGTGTGCCTGGACCTTTCTTCAC
TTAGCCTCTCTGCTCCCCCTCAGGTTCCTGCTCACGTGTGACCCCTTCACCAAGCCCTTCCTGGCCAGCTTACAAAAGTGGCCACTCTTCGAATTCT
CCTACCCTGCTCTATTTTTATCTGTGACACTTGACACTCCCTGATGTATTATGTCAGCTCAGTTGTTTTACCTGTCTGCTCTCACCACTAAATACAA
ATTCTACAAGGATTGACTTTTATTATTATTATTATTATTATTGTTATTGTTATTATTGTTATTATTATTATTTTGATAGGTTTGTCCTTAGAACAAT
ATTGTCGTCAGGCAGAATTGTAATCAGTGGATACAAAAATGCTTTAGGAGCCATAACATATTGTCAGCCCTCAGATTAATCGAGGTGAGCATTTGAG
CCACACTGGGAATAGAGTAAGAGCTTTTATTTTAATATGTGTGCGTGTCTTGCCTGCACATATGTATGTGCCCCGTGTCCATTTCCTGGTACCCGAG
GAGTCAGGAAGAGGACCCCTGGAACTGGAGTAACAGATGATTGGGCCACTGTCTGGATGTTAGGACTAGATCCCAAGTCCTCTGGACGAGCAGCCAG
TGTTCCTAACTGCTGAGCATCTTTCCAGTCCCCAAACTTTAAAAAAAAATAGCCAAAACACTGGGATGAGAATGGGACTGTTCAGTAAAGCGACAAG
CGTGTTCGTGTCATGGGACACATGCCAAACACCGCCACTCCACAGCATGCTTTACAGAGTGGGGGACATTCCTATTCAAATTGTCATCCCTCATTGC
AGTAGCCCTGAAAGGTAGTAGGTGGGGCAGGTTTTAGGTGTGTGTGTGTGGTGGGAGGCTGGGGGGGGGGGGAGTAAGGGAAGGGAAGTCACTGCAT
TCTCATCATATCGATCTGACTCCAAGTCCAGAGCCTCTGTCCCTCATTTTGACGTTCTCTCCTTGTTGGGTTATGACGTCACTGACTTCTGAAGACT
CAAGGTCAACCTTGAACTCCTGAAGGAGCCCCTCCACAGAAGGTAGGTATCCTTTCTCTAGGGAGAATGAAAGTGTCTTGCTAAGAGGACAGGGGAT
AGATAATAAACTTCTTTAGCATCGATTCCAGAGACTCCCCGAGACTCTCAGCTAGCCCCAACGTCTTGCTTGGTCATCTGTAAAAGGCAGTGCCCTC
CTTTCTTTTTGTCCCCAGCCCACCGAACTGTCCTATAGGTGAAGGAGGAAGGCCGGTCATAAAACAGGATGGGCAGTTGCTTTCGGGTGAAACCCAG
AAAGGGTCCTCAGGCTTGTCGCTGACTGTATTTAGATTTAAAAATCAAGGCCTGTGACCTGGAGGCTCTTTCCTGCTGCTCTCTGCCATGTTGACAG
CAGAGAACTTGCTAATTCTGGCCGGGGATTTTCCCTGTGTGGCTGTTTTTCTTCTTTATCTTCATCAATTGAAGAGCGCTCGCTCTGCCTTTCAAAC
TGCCTGCCGGAAGATACTGGGGGTTTGTGTATTTTTTTTTCTTTCACCTAATTCTGTGGTCTTTTCCCAGATGTGTATTTTCTCTCTACCTTCTTTC
TAACTCCAGCAGTCCGTGGGAAGGGAGGCTAAAGCTACAACCTGTATCTTTCCCCCCCCACCCCCCATGTGGCTTCCAGATAGAAGAAAGAAGGCAT
CTGTGGGGAGCAGTCCCTCTGCACCCGAGAGAGAGGAGGACGCAGGGGTCTGTCAGAGGTTGCTGTTGGGCAAGCAGGGGAGGTACCTGTGGAAGGT
GGTGTGCTGGTGGCCCCCTAGCAGTCAAGAAGGGGAGCCAGCTAGTGAGAAGATCGCCCAGAGGCATCTGGGACGGTGTGGGAGAGCCCGGAAGATT
AGAACCATGCCTCACAGCTCCGACAGCAGTGACTCCAGCTTCAGCCGCTCTCCTCCCCCTGGCAAACAGGTACCCCCTTCGTCCCCCTCCCCCACGC
CCCGCATGCCTTGCCTTCTGATTTTCCTGGGGTGGTAGAAGAGGGGTAGGCTCCCTTGTCCTGCCCAGGGAGCTCTGTCTGGGGAAGGAAGTAGCTC
GTGGCAGGAACTCTCTAGCAGACTCTGGACCGTGTGGTGTTTGCTATACAGAGGTCCAAGGGCTGAAGCTTCGCATCCCGCCACAAAAGCCTCTCTT
TCCAAGAGGTACCCCTCGTCTCTGCCACTTAGGTTCCCACCGTGAAACTTTCTAGGAAGCTGGGGGCAGGGTGAATATCACAGAGCTTAGAAGTATG
GTCAACCATAAGCAGAGGAATGTTCCAGTAGATCGCTGCCTCGGGCTCCACCCAAATCCCTAGAGTTGTAGCTTTCCAGGAAAAACCAAGCAAGTGG
CAAGACAGAACCGAGGTCAGGGAAGAGAAATATAGCAAATCAAAGCAAGGTAGACAGGAGGAGAAGAGTGGCAAGATCTGCCTGCGGATTGCCTCGG
TAACTCCAGCAGTCCGTGGGAAGGGAGCTAAAGCTACAACCTGTATCTTTCCCCCCCCCACCCCCCTGTGGCTTCCAGATAGAAGAAAGAAGGCCAT
GGTCCAGGAGGCGTGTCAGAGACTTTCACTAGTAGCTAATATCATCCCTGAAAAACCAGGGATGGAGGGAAAGATTCTGAGACTCTTAGACTCTTGA
CTGATACCAAGCCGAAATGTCTCAGACTGAAAGATGCCTCCGATGGGATGCCTTTCTCACCTTGTGGTTTCTGGGTCTCTCTACCACGTTCCTGATG
CCCCTTGTTCTAGACATTTCTCCTCCTCATCCAACATAGACCCACGGATTAAACTTTTTTACATCTCATCTTAAAAACTATATTCTTTCCTTAGCAC
ACATATTGGGAGGGGCTCAGAGGTAGAGTGTTTGGCTCACACACATCCAGGGGGCTGCAAAACGCTACAAACAAAATAAACGTAAACTACCTGACCA
CCGGCTCCCACATCTGTCACAAGCCCTCACCAAAAGCCATACTGGTGAAGACCTCAAGAGGGAGATAGTCCCTCGCTCACGATGGTATCAGAGCAAC
TCAGACTGCCTGGAAGATGGTACCCCAGTCTCTTCTTAAGAAGGAAATTACTCACAGCTTCCATGGCAACCTAGTCCCTTAGCACATTAAAGGAAGA
ATATATCCTTGCCCTCTCTAACCCAATGTCCGTTTTGCTAGAATGACCTGGAAAGTTTGAGTAAAGAGGGAAGGGAATCAGAACCTGACCCAAGGTC
TTTGCCCCTCAGGCCAGTGGAGGTAGGTGACAGGGTGGGGGTTGCAGAAGGGTTCTCTTCCCTGATCTTCTATAGGAGATACTTAGTTTTGGTCTCA
AAGAGCACTGTGTCCTCCAAAGCACCCGTCAGTGGCTCACTAAAGCAGGCGACCTGCACTTCATAAAATGATTTGGTGGTGGTGGTGGTGGGGTTAC
CTCATCTGTAGCAGGCATCATGTTCAGGACTAGGAAGGTCTCAAGATGTCGCACAATGATGGACCGAATAAAACAGATCTGGGCGGCGCCCCTCTGC
CTAGGGGTGCAGCACAATGAAATTAATAGTCAAGGGGGAATTCTCAGAAAGGATTGAGCATCAGTGCCAGAAAGATGTCATACCCCTGGATGTCCAT
GAGCTCAGGGATCAGGGTGGAGAGCCTCAGCTTCGATACTGTGAAAATAGAAAAGACGGTGCCTGCTCAGAAAGCTGGGGAGTTGAAGCATAGCGTC
TCCGAACGCGCTTGTACAGAAGTTGGCACATGGTAAATGCTCAGCGGAGTTTTCAAAGACAACAGCAAAAGGAGCATCAAGAAGGTTTAGCATGTGC
GCACACATGCACATATATACATGCACACACACTCACACGCAGCATACCTCACATGCATACATATGTATACATCATGTATCACATCACACACACACAC
ACAGAGTGAAGCTAGAACAGCTCTGGAGAGAGCAGAAGTGATGGCATCTTAACTGGAATTTCCCAAAGTAGCCCTCCTGAGGCTTCAATTTCTCTTC
ACTTTTGAGTAGCTTTTGATCATAAGAAGACCATAGGAGACCCAAGGGTGGGTAGCAGGGAATGCTGAAGAAAATGTCTTTGGGTACAGGCCTTTTG
TAAAATAAGGGGAATGGACAATACAGAAAGAACTGAGGACCGGGGAAGGAAGGAAGGAAGGAGGGAGGAAACCAATGTCCTATTTGACTAAGAGGAA
CGATCAGACCTACCGTTGATATCTCTGCTTCCTAGGACTCATCTGATGATGTGAGGAAAGTTCAGAGGAGAGAGAAGAATCGCATCGCTGCCCAGAA
GAGCCCGACAGAGACAGACACAGAAAGCCGACACCCTTCACCTGGTGAGTACCCACACTTCCTACCATGCTCCTGCCCTTAGGCTCTGCGCCCACAC
TGGAAAGGGCTTCGCTCTCCCATGCATGTATGAAAACATTTAGTAGCAGAGAACAGCACTGGACACTGAACCTTTCTGGCCCAGGAGACTGCAAGGG
TGGAAAGGGCTTCGCTCTCCCATGCATGTATGAAAACATTTAGTAGCAGAGAACAGCACTGGACACTGAACCTTTCTGGCCCAGGAGAGTGCAAGGG
TAGATGTGTGGGATGTTGACAGCCTCAAGGCTTCATGGGAGGCAGAGCCACCTGTGTGGAAAGGGACAGCAGAGAGGGTGTCGTCCTTCCTGAGCCA
CTGTGAGCAGAACACTGATGCTGAGAGGAAGTTAGGTGTTAAGTGCTTCAGGGAAGCCATTCTTTCAGAGCTGCACATCTCATCCCTCTGGAAGGCA
ATGGTCTGCCACCCAAGCACACTTGCCTTTGGGAGAAGGAGGTCGATGATGCTGCCCTTATTTTTTTTCCCTGAGCATGGAGAAAGTTCTGTGTTGA
GATTTACATTGTTAATGGTCTCTCTAGGGTCTGCTAAAGACAAAATTTTAAAGCACTTACTTGGGACAATCACCATCCTGGTCTAGCAGGGAGGGGA
TGAACTGCAGCCTCACTTGGGAGACCCAGGGCGTGGACCAGCGACCCCGAGTCCTGACTGGGCGCAGTGTTCCCACCCTTCATACCTTTCCTCTTAA
CAGTCTGCTCCTTTCACTGTGTTCTTGCTCAACTGGTCCATCTTCATCGCATCCTTCTTCCTGCTCTTCCTCCTTCCTCCTGAAACATTTCCAAACT
GCTAATATTCTAGAAATTTCCTAAAAGTGCAAGAGATGCCAAACCCAGATAAGCTGATAATGATCTGATGTTTAGCATGGCTAAGGGCTGCTTTAAT
TTTATCAGGGATAAATTATCGGGGGCTGGGGAAATGGCTCAGGGGGTAAAATGCTTGCCATTCTAGCATCAGGGTCAGAATCCCCAGAACCCGCATG
ACAGTCAAATGGGCAAGAGGCAGAAGCAGCAGGTGGACATCTATGAAATTGAAGCCAGCATAGTCTACACAGTGAGCTCCAGGATAGCCAGGTCTTC
ATAGAGAGCCCCTGTGTCAAAAGAGAAAAAAGAAAAAAAAGAAAGAAAAAAAAGCTGAAAAGCTTAATGGATGTGGCAGCCTGACAGTGTAATCCCA
GCTCATGGGAGGTGGAGATGGGGAATCTTAGAGTACGTTGGCTAGCCAGACTAGTCAAAACAGTAAGCTTTAGGTTCAGCAAAAGACTCTGCCTCGA
TTCTCAATACGGAAGTTGGAAAGTGATGGGAGAAGATACTGACCTCAACCCTGGGCCTCACACACGCATGTGCATCTATGTACACGCGTGCCCACAT
ACAAGAAAAATACATGGAAACACCACACGCACATACACATGCAACAAAAGAATAAGTAACATACAGTTTTAGAAGACAAAGGGGAGGATATCGCCAC
TTCTTTCAGCCTAAGGACAGAGAACGGTCTGCCCTAGTCGCTCTGCCTGTGCTTCTGCTCTGTTTGATAGGTTCTTAATATAGTCTCCAGTACGTGC
TAACATCATTTTCAATTGGCAAATATGTGTCAGACTAACAAGAGAAAGAATAAACAGCACTCAGCACAAACAGACCAACAGTCTGAGTGTTCAGTGT
CTGTCCCGGTACTCAAACCCTTCAAGATTTCCTGGGGGCTTAAAAATCTCACACTATCTGAAACTGACTTTTTCTATCTGAGTCTGGTTCTTAGTTA
ATATTCCATAGAGCAAGAAAGCCACACTTACGATAAACTCCGTTTTGTTTGTGCAAGTAGACAATGTCCTTGATGGGCAAACTCCTGTAAGCTTTGT
AAGTTTGTAATATTAAAAGAAGGAAGCTGAGTTCAGCCTCTCTCTGGCAGGGAGGCCAGACTTGAACCACCGCTCTGCCGACTGCTGTCTGTGTGTC
TGGACCCACAAGCATTCTTCATGGTCCTTAGCCTTGGGGCCCTTATCTATAGAATGGGAAATGACAGGAACTGCTTATAGAGACAGTGCAGGAGAAA
AGCTCAGTATGGTGCCTGGCATACAGTAAATACTCAGAAGATTCTGTGCAATTAAAGTTATAGTAATAGTGTAAGAGGCGTGAAAACTGAGACTGAG
CAGGCATACATATGTACATTGCATAATTTTATATAAAAATGTACGTGTATTTCCCAGTATGTATATATGCACATATTACATGCATGTATTGTGTGCC
GTGTGTATTGTGTGTGTATCTACCATGTGTGTGTGTGTGTGTGTGTATCGTATGTATGTGAGTATGGGTGTGAGTGTGTGTGAGAGATGCATGCATA
TGTGTGTGTGTGTATACTTTTTCTCCACATCATCTCAGTGGTGAGAGACTTCTTAACAGCTGTAAAGCTCATGAGACTCCCTTGCCACACTCACTCC
CGGCAGAAGGTCCCCAGGGCACGCCCAGTCACCTCTAATAGCAGAGTGCTGCCTCCCTCTTGACACAGGAGAAGGGCCTCCTGTCTCTAGCTGCAGG
CCACCTTCCTATAAACTCAGCCCCCTGGTCTCTGTTGCCTAACTTCTTCTGTGAGAGCATGAGAAACTTACAATGAAGCATTTTGAATACCTGTCTT
GTATTCATGCATGGCGCGCTGATTGTTTTTTAAGTGACAGAGGTGGGGAGGAGCCTCCTGCGACAACTGTGTTACAATGTCCCCAGCATTGATTAAC
TGCACATAATTCATTTGTGATTCTGTTCCGATTATGAGATATACGTGATGGTTTCCCAGACACAGATGTAAACATAAGATAATTAGGATGGCATATC
AACCTAGTCAGAGAGGTCGAATTATCTAATAAATAGAGTTGGGACACCTGGCAAGTCACTTGAAGAGACAAAAGGCCAGGTCTCTCATTCCTTATGC
AATGCACAGACAGTTTAAATATTTTTAAAGGAAACTAAAGAAGTCTTGGGGGAAAATATGTAATTTGGAAATGTACCTTGGCATGAAAATTTTTCAA
ATAAGATAGATAGAAAATAATAAAAGAAAATTGATATACAAATGTGATTTATACATGCATCAAAAAATAAAAAAATTTGGCTGGGCATCCTAATTGG
GCTAGGCCTTCAATCCTAGCACTTGGGAGACAGAGGCAGGTGGATCTCTGAGCTCAAGACCAGCTTCATCTACATAGTGAGACGCCCATCTTGAAAA
AAGGATGAAGTTTACCCCCCCTCTTTCTTCCTTTTCTCTCTCTCTCTCTCTCTCTCTGTCTCTCTCTCTCTCTCTCTCTCTCTCTCTCCTTCCTTCC
TTCCTTCCTTCCTTCCTTCCTTCCTTTCTTTCTTTCTTTCTTTCTTTCTTTTTTTTCCTTCTTTCTTTCTTTTTTCCAAGACAGGGTTTCTCTGTGT
AGCCCTGCCTGTCCTGGAACTCACTCTGTAGACCAGGCTGGCCTCAAACTCAGAAATCTGCCTGCCTCTGCCTCCCAAGTGCTGAGATCAAAGGCAT
ATGCCACCACTGCCCGGCTGAAGTTTTTCTTTCATTCTTCAAAATATTATATACAAAATCAAAGGCAAATGGAAAGCCAGGAGGGAATCATCTAGAA
AATTCATGAGCCAGAAAGGCAGGTTTTACTACATATGTGGGAACTCTATGGCGAGGGAAATGATTGATCTTTATTAAAAAAAAAAAAAAAAAAAAAA
AGACTATAAAGTGAGGGAAAGGGAGAAACAGAATGTACCTACACATGTAAGGATGCGCCAGGCCTCCTTGGGAAAATGCCAATGAGGGTGAATGTAA
GACTTCCTTGCCAGGCTGACAGAGGTGAAAGATGGAGCTGACCCAGGAAATGGCCAAGGGAGACGGAGAGTGAGCATCGCATTCCCTTAGCAGGGCA
AGTTGCTATAACCCCCTAAGACGGCAGCTTGAGCAGGCTCACTCAGGTGGCAGGGGCACCTCCCCTGTCTCCCAGCAGGGTGCTGTCCAGAATCCGT
CCACACCACAAGCACAGCACAGAGGTTGTAGCTCTCTGAGCTGAGTGTGAGTCATTGTTGCGATGTGCTGCTCTTGGTGTCTCTTGGTTTTTGTTTA
GTGGCACTGGGGATTGAACCCAGGGACTTGTACACCACTGAATGACTGGAAACTAGTTACATATTCATTAGGAGAATGGGGGTTGAATGAACACTAT
TGGTCCATTCTGAGTGTGCTGTTATGGGACTGTTAAGTGAGTTGCTTCTGAGTGTGGTAAAACTATCGTTTTCTTTTCTAAAACAGTTTTTCTAAGT
TATGGAAGGTGGGGTATGTGTACATGAGTGCAGGTGCCCATAAAAGACAGAAGGGGGTATTGGTCCCATGGCCACAGGTACTAGAAACTGAACTCGG
GTCCTCTGCAAGAGCAGTAGATGCCCTTAATCACTGAGCCATCTGTCCAATTCCTCTTTAAAGCTTTCTATGTTGCAGTTATTTATTGTTTGTTTGT
TTATTGTGGGGGAGTGCATGCATGTGTGGATGAGTCGGTTCTCTCCCTCCGCTGTGTTGGGTCCGGGCATCGAACTGAAGTCATAAGACTTGGCAGC
AAGCAGCCGGGCGTGGTGGAGCACGCCTTTAATCCGAGCACTCAGGAGGCAGAGGCAGGCAGATTTCTGAGTTCAAGGCCAGCCTGGTCTACAGAGT
TCCAGGACAGCCAGGGCTATACAGAGAAACCCTGTCTTGAAAAACCAAAAAAAAAAAAAAAAAAAAAAAAAAAAAAAGACTTGGCAGCAAGCACTCT
CACCCCCTGAGCCCTCTTACCGGTCCCTGGTTCCCAGACTCTTAAGGAAGAAAAGTGCAAATGTAGCATCTAGCTTGCGCCAAGCCACACTTTTTAA
TCTGTCTGAGCATAAGTATACAGGCAAGTTTCAAATGGATCACCCGAACTTATTAATATCATTTTCAAAAGGTCACACTGAAGTTAAAAGACAGGAA
AAGTGTCCTTTGCATTTTCAATCCTTTTTTATGTCAGTTAGGAGTAGCTAAGATTTGAGTGACTCTAAGGTCCGTGGTACTAGATGGAACACTTTAA
AGAATGGCAAGTCGTTTTCCTGTGGCCAGCCAGCCAGTTGAGAGTGGGAGCCTTCTGGAATCTTCTATCATAGGGGGATTTTGAGTCAATGTCCTGG
GTCAGGGTTGGGGTGGGGGTGGGGGTGGGGGAGAACTGAGATCAGTTTAAAAGCATTGCTCTGGAGAGGGGCTCCAAGCAGAGGAGGTGGGGGTTGG
ACAGCAGAGTCTCCCAGAGTGTGCTGTTGGCAAGTGGCGCCTGGTCTCCAGGTTGCTTGGCCATCCGTGCTGGATCTGGAAACTGGAGTACGAACAG
GGTGGCTCCTTTCTGTGAAGATGAGGCAGGATATGACTGCTGTGGGCTCCTGATTGTAATCTCACCACAGTTCAGGACAGGTTGTCTGTGATTTTGT
AAAATCACATAAAACAGAAAAATCATGTAGACTGAGCTGATGCTGTGAAAAATGAGGACCATTGGAGGAAATTAAATAATTGAGAAATTCCCAAAGA
TGTTAGCTCCCAGGGACACTGGGAGCAAGTTTTTCAGCAAGGCCATTGCTCTTGGCTTGCTGAGGTGGCGGGAAACGAGGCTGTGAGGTTCCTCCAA
CATTTCTCATTCTCTGTCTCTAGGAGACCACATCCTGAGGTTTATTAACATAACAAATTCATAGATCTGTAAAACAATAAAACATGAAGGGACATTC
TGGCTAGTTTCACCTTTTAAATGCTTAATCTAGAGCCAGTGAATCAGCCGCCTCCCTGGCATCTCTGCCTCTCTGAATTTCTATTTAAAATATGGTC
ATTAGCTGGGCATGGTGGCGCATGCCTTTAATTCCAGCACTCGGGAGGCAGAGGCAGGCGGATTTCTGAGTTCGAGGCCAGCCTGGTCTACAAAGTG
AGTTCCAGGACAGCCAGGGCTATACAGAGAAACCCTGTCTCCAAACACACACACACACACACACACACACACACACACACACACACAAAGGTCATTG
AACACCTGGCCATGGTCACGCTAAGTGCGTTAAGTGTCCTACCAGTGGGGTTGCACTTGATCTGAAAAAACAAATTCTTTGCATTAATCAATCTGCT
GAAGGTTGGACAGCTTGAACTTCAGAGCCCTCCTTTGTAAACATAAACATCTATCTGTATCCTTGCATCTCCTGCTAAGGTCTTGTTTGTCTTGTTT
CTATTTAAACAATACACTCTGAAATTCTTATTTTTCCCTGATCATCTTAGGCTTGGTCAAGAAAGTAAAGAAAGGTCTATCTGTTGCTGTCTGTCTA
TCTGTCTGTCTACCCTCCCCCCCTTTCTCCCCTCTCTCTCTATCTCTCTATCTGGCTGTATGTGTGTGGGTATATATAGATCTTTATATAAATATGT
GATACATATGTATGTATAATATTTATACACTAGTTTTTAGAAGATTGTTCCATTTTAACAGTGTACACAAGAATAACCTTTGGACACAGTCCTTCTC
ACTATAACATTGTAAGGCAAAGTCCATAGATGAGGATGCTGGAAACATGGATATCTGAACAGCAAACACTTTATGGGTGTCATAAAATTAGGCAGAT
GGTTAACAGGATATTTAACAGACTGTGATCCACTTACGCAATAGAAATTGAGTCTGACACTAGAAGTGGCCATGGAGATCTTTGTGTATAGACTTAG
AAAGATGTCTGCCAGGCTGAATTGAGCTAATCAAGCCAGGGGAGTGTGCAGAGCACCATCTGCTAATGTGAAGCTTGCAAGGTTTGGGGATGGAAAC
ATAGTCTCCAAATTACCAATGGTGGTTACTTCTGAGTGCCTGGGTTGGGGTAGCTTGCCTTCCTCCTGGTGCTCTTTGCAGTGTTATTGTGACTTAA
ATGTGCTGTTTAAACAATCAGAAATAGTCCCAGAGTCTTTACAAATCAGCTATTTTAGTTGGGGAAGAACACACCAAGGCAATTTACCACCTCAACT
GCTTATCCAAGGACGATGGGCTGCTCCGTTAGGGTGGGAGGGCCCCAGACTTGAGGAATGTGTGGCTGAACTGGATTGTTAGTACCAGCTCACAACC

TABLE IX

MOUSE NOMENCLATURE
ICSGMM Irf4
Celera mCG4922
HUMAN NOMENCLATURE
HGNC IRF4
Celera hCG20902
MOUSE SEQUENCE - GENOMIC
ATGCTCATCATCCTTGAGGGCACACCTCCCTTGCTTTTGCCCTCTGAATCACAGCCTTGTCCTCCTTCAGCATGTCCTATCTTTCCATATTTTACAC
CTCTATCCAGGCTGGTTTTTGCAAATTTGCCTGCCTAAAATTCCAGTCGAGCTGTATCACTTGTAAGGATAAACTACTAATATATATGCCTTGCCTG
TTCTCTTTCACTCATGAATGAACAGTCCTCACACTCCTATCCCCCTAATATCTAACAGAGATGCAGCTCCCAAACTCTACAGCCTGAATCCCAGAGC
CTGAGCTAGCCCACGAATGCATCCAATCCCTGTGGTGCAGAAGAACCCATGCTCTGTCCAAATCAATCACCTAAACGGTTTAGAATTTCTCTGTGGT
CTTCTCTTTCCAGTGCCTAGGTCTCCACCCCTACCTCTGACTGTTTTTTCTTCTACTAGCATCCCCTGTCATACTCTTAAGATTTCTCAACCATTCT
AGTCGTAACCTAGCCTGTTCTTTTCCTTCATGTCTTATATTGAAATTCTGTGACTCTGAGGTTCAGAACAGAAGTCTTCTTGCTGGGGTTGCCTATG
GTTCTCTCAATCATTTCCACTCCTTCCCTACTTGGTACTTTCCCAGAGAATGATACCTTTTGGGCAGTGAGTGATATATACCCAGTACTAAGCTGAG
TCACTGTCAGCATACTCTTTTAGGCAACCUTGGGTCTTGGCCATGCCTCATTGCCTTTTCCTGTGCCTCACAGGAGATAGAATAAATCACAGGACTT
TGGAGAACGTCACAGTGGTCCATGCAGTTGACCCTTGTAGCTTGAGCTTTGGGTCTCAGAGTGCCAGAGCAAGGGAAGATAGAGCACAAAGATGTGG
AGATGTGGAGTCACAGCAACAGTGTCACCAATTCTAGAACTATAGTTGCAAGCACTACCTCTGCTGAAGGCAGTTGAATACGAGACAACATATCATT
GGCCTTTGGAGCAATGCTCAACACCACCCACGGCAGGACACTTTGCAGAGCAGGGACATGGAGAAGCAGAGAGGTGTTCGCTGGACTTGGGAAGGAT
GCCTCGTAGCACAAAAGAGAAAGGAAGAGTGTATGAAGGGCAGGCTGGATCTTCCAACACAAGAGTAGGGGCACCAGCTTACTTGTCTTTTAAAGGC
CTGCCAGCACAGTGAGTCCACATGCCCTGGAAAGTCATGTTTCTTCTTAGGTTATATTGTGGCATGCTGGGATGCTTTCTCCTGCTGGGCAGATGGA
AAAGACAGTAGATGGTTCTCCTTGTAATTTTTAAGTTTCATCTGATGTTGAATTACCCCCCACCCCCCAGGATGCCTGAGTTCTACTCAATAAAGGA
GGGGCTAATTTAAAACATATTAAAAGTCAACACATTAAGTTTCCAACGTTCTGTGTAGCTCATTCCCTCTAAGCATGCCTATCTGCAACCCAGAAGA
ATCCATTGTTTTGCATCTGAACACACTATCAGGGACTACAGTGCACCATGACTGTTCTAGGTCTAGCAGAGTGTATCTGGTGAAGAAGTCTGCTAAG
AATACTCGCTGCTGGCCCTCAGGGGCTTTTCTTCTGTGTGGACTGGTTTCTTTTATGCTCAGTGGATTATATCAAATAAGAACAATATATTCTGTAA
TCACAAGTAGTCAAGCCTGTGTACTTATGCAGAGGAAACTGCCTTTGGATCATGAGTTTGTAAAGAGTTGAGGGAGAGGCACAGTGTTTAGATCAGA
AGGTGATCTCCAGATGACGTTTTACATAGCCCTGGTTAGTCTGAGCTGAGGAGGGGCCAGGTTTCCTTCAGTCGTGCTGAAGACTTCAAAGGCTGGG
CAAAGAGACCTTTATAAGAAGATGCTTCTCAAGGCACATGAGAGAAATAGTACAAGGAGACCCGGAAGGTAGAGAAAGAGGAAAGGAGAACAGACTG
GCTCACAPGCTCAGAAGTGAGGACGACTCCATCTCACAGGGGAGTGCAGGGCATTTCTGAACAAGTTTGTGCAATTCATACTCCTTGTGGTGGCTAC
CTGATATGAAACAACAACACATTTTATATAAAATAATCTAAATCACATGTTCCTTTAGAGATTGCCAAAATGTTTTGTATACTGTCGAAAACTAAAA
ATTGAATGTCCACAACATGACCTATGTTCATGCCTACAAUTCCATTAGTACTTTCACTCAGTCTTTAACGTTTTGTATACTGTAAAAACTGTTCCTA
AAATTTGAATGCCCCCAACATGAGCTCTGCCCGTGCCCATAGCACCATTAACACTGCCACTCTGTCTTTCCAGAATTCCAAAGCTTGAGACAGAATG
CCTGAGGAACATCAAAATGCAAAGCTTGATGAGTACCTGGCCTTCCTCTGTAGTAGCTTTAAGGGAGACAAAACAAGCACCCTTTGATGGACTCCAT
AATTGTTTTACTTACTCTTGGACCATGGAAACAAACTACAGATGGGGCCATGGGAACAAACCACAGATGGGGCCTGAAACCTCCTCTGAGAAGGGAC
AAGTGGAAAAGGAGACACCAGATGCTCGAATATCACATTAGTTTCTCTGTGAAAGAAGGTGGTCAATGTGGGTTGCAGGTAGGGCAAGGGCATTGGT
AATTGTGAACCATGAGGTATAAAACCAAGGGGACATAATACTTCTAAGCCTGACTTCGTCTACTTGGCTGGGCTTCTCAAGGACCAAGATGAGTCTG
AGGAGAGGAACAAATGGAGGTAGACAAGGGGATCCCATCCTGGGGGGTCAACCAAGGGAGGTTACATCAGCAGGCCAAAGCCTAGAAACACAGGACA
GTCTCTAAGTGTGGGGTCAGGCTGTCCTCAATCCCAACTGGTTTCATTGCCATCTGGGCAAGTCCAGGTGACCTCCCTGATGCCACCATATCCTTTT
CTGAAATAAGACCATCGTCCTGCCTCACTTCCCTACCTGTTCATAATAAGAAGTGATTGAGAAAACTGTAGTCTGGTACAGTACAACTTCTCTTTGG
AGTAGCAGACATGAAGCCCATTGTCTGCAGGCTGCCACTGAGCTGAGGGCTGGGCTAACCTGGGATTTTCCTCTACCGTGCCCAGCCTGGAGGAGTT
CAGTACTTCTAACAGAGTTCTACTCATAGGTGGTTTCTCATATGGTAGTTCCTTTGCCCTCTCTGACTAGCCTCATGCCTGTGTCTACTGTGGAGAG
TATCTGCCAAAATCTCAGGGAGGTGCAATGATCTCACCCAAGTCATATTTCATTTGCTGACACTCACTGGCTTATTGCATTATGGATAAAAGCACAC
GAGACACGCATGCATCAAACATTGTACAGCTCCCCAGTAGTACACACAATTGTTATGTGTGGGGCTTTTTTAATCACCTAAAAGATAAGTTTAAAA
TGAAAAACGTTGATCCCCTAATGTGGGAAAGAGCTTGTGTCTATTTGAATGACATGAGAGCTAAGACTGCTTTGGATCAGAAACCACATCCTTTGCC
TCTCTCTCTGCCTCAGCAGAACCAAAGAAGCTCAAGAGACCCGTTTCTGTGTGCTCACGGTCATCAGATACAAGCAACGAAAGTTCTAATGCAACTG
TGAAAGTTTCTCTTTTTGTCCCTTGTTGTAGGATAATCTGCATTAACAAACTGTCAAGATGTTGAAAAGGAGATGTGGGGGAGAAGGGGAGTGAGAC
AGGACAGGGAGGGGAAGCAAGCAGGTGAACTCTACACAGAAAATAAGAAAGGTAGAAATTCCCTGCACTGCACTGCCCTGTGTGGATGCACTGTCCT
GCATGGATGCAGGAGAAACAGCCCAGGTGGCCCTGGGGATGAAGGGCTCTTCCTTTGCCTGCAGAGGGAACTGCCATTCCCAGGGGCCCTCAGCTCT
TTCTATGATGAAAAGGATAGTAAGGCTTTCCTTCTGCAAGTCACCAGTGCTCAGATTATCTGCACCTCTCCCATCCCATCCTCTGGCTGTCCAGAGA
GCAACAGGCAGGGTCAAGGGACTAGATGGCCTGGCTTAGGCTGTGCTGTGCATAGTAGAGGCCTCTACAAAAAGGACTAACAGTACAGATGTCTCCA
AATTACCTTAGTTAGACTTTATATCTTTAAAAAAAACTATAATGGCATGAAAATGATACACATCCATTAAAATTATCCTTCAAATTCTGAAGACAGG
CAGTTATTGTTCATACAATGCAGAGGGCCATGGOGACCCACTTGGAGACCCTTAGTTCTTGTTTGGCAGTGTTGATGGGCAACTCTAAAGGTAAGTA
TGGAATCTTTTGAACAAGGGCTTGATGGACAGCAATTCAGGTTCATCATGGCAAAAAGCTTGTTTTGTGATGTCCTTGGTTGAGCAAAGGGAGAGGC
AATCTACTAGCCCACTTTTCATGGTCTCCTTAAATGCCTGAAACAAGAGAGAGAGAGAGAGAOAGAGAGAGAGAGAGAGAGAGAGAGAGAGAGAGAG
AGGGAGAGAGGGAGAGAAAGAGGGAGGAGAGAGGGGGGAGAGAGAGAGAAAACACAAATATTTATGTTCTATGAAGCTACTTTGTCTAAGATCTATA
TGTATATCTGGGTGCTGTGATTTGAATATCATATAAATGTATATATGATATTCAAATCATATATACATATATATTTATATATGTATATACGTATATG
CATGTATGCATATATGTGTGTATATATGTGTATATGTATGTATGTGTATATGTGTACATGTGTACAGTGTGCATGTGTGCATGTGTGCATGTGTGCA
TGTGTGCATGTGTACATGTGTATATATGTATATATGTATATGGGCTGGGAGCATGATGCTTACAACTACTGAGAGGTTGTTTTGCTGAATCATAGC
ATGACACAGGACACTGTGTGGTGATACAAAACAAGCAGCTAGCTAAAGCCTTTCTTTTTGATAATGCCTCTTCTACTAAGAAGCTTGCTTAGGACTC
CAGCCAATTCTAAAGACCCCACCCCTACCAAATGCATTTTCACTGAGGAATTAAATTGCTAACCCACAACTCTGAGGGAGGAAACATTCAAACTACA
GTATCTGATAAACATTTGATCCTCAGACAAAAGAGTTTCACATATTACTTGGAGCATACTTAAACTGTACAATTATACCCATTTTATCAGAAAAAAA
AATCCAAATATAATTCTGAATTACACGCTTTTATGTACCACAACTGGCAAACCTGCTCCAGGCATAGAGGTAGGCAGGATGCCTAAGGGCGCGGCCC
CTGGAAGGTGTAACAGAAGTGGATCAAAGCAGAACAAGGCACAGACTTTGGAGGACACTATCTCATGATAGCCAGATTTGTTTCGAGAGAGTGATAG
GGTACCATTTCCTAATTTCTGCACCTGGATCTTCCCACACCCATCATTTGGCCTCAAGGCCTCTTCCTTACCGTTCTGTTTCCACCTGCCTGCTCTG
GGTACCATTTCCTAATTTCTGCACCTGGATCTTCCCACACCCATCATTTGGCCTCAAGGCCTCTTCCTTACCGTTCTGTTTCCACCTGCCTGCTCTG
TGTGAGAACGTGTCTCTCTCTGTGGACTCCTCCCAGGACTCCTGGTCATTCCTCTCTGTCTGACACGGCCAACCACAGCCAGATAATTCCTCTTTGG
CTGGGGCTTTCATCTCCTAACAGTGGATGTGGTGTCCTGGCGCTGTGAATAACTGATAATTGCCTGCACAATAAGCCAGTGTATGTTTCTTCCTGAAT
CCTTTCTCTTTGACTGTGATCAAGCATGTGCCAGACTAACCGCTCCTGAGTAGCCCATAGGAACAAGAAGGCACTCACACCCCGAGCCTCAGGTTGA
CCATAAGCAATGCAACTTTCCCTGAGACATAATCTCTACCGCCCCGTGTGCCCCAGCATCACTCCTGTGTTTTACAGTGTTCTGCTGAAAAGAAACA
TAGCAGATACAGTTTAGAATAATCCAGAGCCAAGCCACGTTGCCAATGGGTGAAATGAGACAGTGCCATTTGCCTTTCACCTCTTGCTTCAGTTTTC
GAAAAATTGTCCCCAGTGATTACAGAGTATTTGCATTTTTGTTGGCTTGGGAACGGGAATTGGCAGCATCAAGTGTTTCCTTGCTTAGAGCCACGTC
ACTATTTACGTAGCAAACATTACAAGTCAGAGAACGGAGCATTACAGGGAAACTTCTTAGAATGCTCACAATCAAAGGAGACAAAAAGGGAACAGTT
GGTTGTTTATAAACATGACGAAGGAAGAAAGGGACAATGTTGCTCTAGAGTCACAGACTTTGTGACTCCTTATTACATGATTGATGAAAATGACAAG
TCTCATCTTCTATAGGATTGAATGTGGTTTTTTTTTCTACCATATGCATTGACTCCTGGTATTAAATGTGTTCCCAACAACTTCrCCTCAATGGCTG
ATGATCTGGGGATGTTTGATACTGGAATTGTCTGAAGTCACAAAACACTTAGAGGATTTTTGCAATTTCAGTTAAGGTATATTCGGGACCTTATATA
AAATAAATAGTTGTATTGTTGCCCACAAATACATCACTTTACTAATAATGTTCAGTTTAGCTCTTGGGATCCATGCCCAGCTTTTTCACATGGATGC
TGAGAATTGAACTCAGGTCTTTATGCTTGCAGAGTAAGCACTCTTACCAACTAAGCCATCTCCCCATACCCCTGACTTTAACCCAAAGTGGAGCAA
ATAGATGCATCCATGGGTAAGACAGGTTCACAAAAGCTTACTTCTTGGGCAAGTCACAGTGATGGCTAGTGGCCTCTCTGGATCTCTCTTCTTATGT
GCACAATTGGACTAAAATGCGTTGCCCTGAACACCATACAGGTACATTTGAAGGGTGCATATCATTATGTCTGTGACAGTTCCCAAGGGCTAATGTA
GGATATACGTAATCATAGTCTAAGTGGGTGAGCACTTGCGATGCACATAATTTTGTTGTCAGAGCACTGCAGAAAGCAGGGTCACCGCCACTCTTGA
ACGTGGGAAAGTGAACTCACCACCCACGCCTTCACCCCTGTGAACTCTAGACACCTCCTCTCGTCCATGTATCATGAAGTGGTTGTCAGGGCTCACA
GTGGGCGTTAGTCCATGCACCTGGAATCAAAGCATCAGAGGTTTTGTCTTCGTTTTAAATCAGAGGCAATGCATTAAAACAACAAAGTTTTTTTCTT
TCCTGTTTTATTTTTTAATGTTTTATAAAATAATTTTTCCCAAAGATAGAATTTTACTTAAAAACAAAACAAAACAAAACAAAAACAAATGTAGTTC
TCATTTTAAAAGAGTGAGGTCATCAGAAAATACATTTATCCAAAACAAGGTCTCCGTCTCTTGGAAGAGAGAGTACTGGGAATTGAACCCAGTGCCT
TCCACACACAAGACAAGCATTCTACTGCCACGGTACACCCTAGGCTGAATAATGTCTTTCTGAGTATCTAACACTGACTGGCTCTATAAACCACTTG
TTCTTACTTGTGAATGTATAATTTTAAAAGGTCAATTCTCAAAATACTGAGCTGCAGTCTAGCTTTTCTTACCGTCTTTTTATGGGAAGTATGAATG
AAAAGTTTGCCCCATTCAGACCTCGATCACTGCTCAGTGGCTCCTTAGCCCAATAACATTAGTAATCTCCTCGGGGAGTATGGAGCTCTGCCTTTGG
AAGAGGATGGGAAATTGCCTACTGGCAAAATTGTCCCCATTCTTTCAAGTCTGACTGTGATTTAAGAAGTTCTTAGTGGTCAGCTAGAAGGGCCCAC
GACGGATGCTCAATTCACTAATTTCCTGTGACATTTTTCAAACAACAGTCCGCCATCTCACTGTGACATGTATCGATTTTCTTAAGTGTCAGTGCGA
AAGAGGATGGGAAATTGCCTACTGGCAAAATTGTCCCCATTCTTTCAAGTCTGACTGTGATTTAAGAAGTTCTTAGTGGTCAGCTAGAAGGGCCCAC
CTGGCCATTCCTTCCTCTCCACCAGCAACAATGGGAGCATGTGATTCACAAGGGAATCACATTCAACTAAAAAGAGAAACCGGGGTATGCTGTTTGC
AAGGAACGGTTGAAAOTGGAACTCAATATGTCGTGTGGTGTGAAATAAATGTGTGTCTCACATGTTTTCCCATGCTGGGGGCAGGGGTAAGAAAGTA
AAAGGCAGACTGGTTAAAGACATGGGGTGGGGAGGGCTGGAGGGACGAGTGGTAAGAAATGGCGACAGAGGAGATGAAGGTAATGTCATAATGAAAC
CCATCACTGCTGTGTGCAACTAATAGATGCTAATAAAATAGGAAGTTTTAATGATTTAGGTAGCTTATTGCTTGCATTCACCTCACTGTTAAACTAT
CACTTCTGGGGGATCCACACAACGAGCGAGCGAGTAAACCAGAAGATGGCGTTGAAGATTAGTAATCATATCTTTTAAACAAGATAACCATGTGAAG
TCTCAAAAGGTTTCTTGTAATGACTCTTGTTTAAACTTCTGAACAGAGGATGTAGATTGGCTGAGGAAAATGTTGAAACCGCCTAAGTCAAGGTA
GAAGACACGTGTGTCTAAGTGAAAAAAAGAAAAAAGAAAAAAGAAAAAAAAAAAAAACCAAAAACCTCGGGTTGGCTGCTTCTGTCCTTAGTCTGTGCACGCTT
TGAAGAAATGTAATTCCTCAGCAGCAAGGCTGTGCTATCTGAAGCTACAATCTCTGCTTTGCTCCGAGGTGTGTCTCTGGTGACCGGGATAGTTCCC
GACAGACAGAAGGTGTTCAAAGAATATTTTTGAATGAATGAAACCCCAAAGGAAGAAGAGGSGAAAATGGGTGTGACCAAAATTTTCTTTGAACGAA
ACTCTGTTGTTTACTACCAGGGCTCTGACAATGGAAAACTAATTGGGGTGAAAGAACGACATGGCATCCTGTTAATTTCTGAGAAAGCCTGTTGATG
TTAGGAAAAAAAAACATGCCGGTGGGCATCTCTGCACCAGTTTTCCTGTGGCCAAAATCAGATGTTTCTCCTAAAGTCCAGAACCCAGGATGGAAGA
TTAAAAGAAAAACTGAGAAACATGTGAAATGAAAAAGTTGTCAAATCTTTACAAACGCTCCAAGTTGACCTGTGGTGGTGGTAATCTAAAATGATAC
AGAAACTGGTAGTCTGCTTGCTTACCTGAAAACACCAAGATAACATATAAGCTCCAGGCATCCAAGCTGAGCTGGAGAAAGTCAGCGGCAAAAGCTC
ATGGAGTTTACATATGAAGGTCAAAGAAAACACGAAAATAAAGTAAAACCTTCAGTCAGCCTAGCTGTTCTATTTGGGGCATTGGTACCTCACCGGC
AACTGCCTCCCACGAGGCTGAGGTTAAAATTATCATTTTAAGGTGAATTGACATCCGGAAGCGCGCTAACTACCTGAGTACTCAGGGATCCCCCATC
TCTTTTATGTTGCCATGATTGAAACTTTGGGGACTGTGCTTGTCTGAGTCATCTCAATTCGTCGGTTTCATTCACCCAACATGTATAAGCGTTTCAA
ACACAGTATTTGGGCCACGGCTTATAAACTTGCCTTTCTATTTTTCTTTTTAGTCAGCGTGATATTCTCTAAACGCTCAGAGAGACAAGACTCCGCT
TTGCCCAAGGATGCTCCCGACCTCTCTCAGTCTATCTCTTCTGTCACATGCTGTGAGAACCAAGTTCCCTGTGCTCCAGACTCTCCATCACTTCCCA
ACCTGGCCATGAGCAGTTAGTAGTTATCAGCTATGCTCAGTGCAGATTCCAGTATCCCCTTTGCATGCCTCCACCTTCACAGGAACGGGCCATACCG
ACTTGTCCCATCCGGTTGAGGATTTCTGAGTACATCAGAGTCCCCAGCCCCCTCCACAGGAGGAGCTGAAGAAAGCCAGGGTTTGTCTGAAGTGGGA
CAGCCCTTGACCOGTGGGCTCTAGTCCGAAGCTCCTGTTCCTGCGGGACACCCAGGCACAAGGCAGAGGTGGGGGGCGGTCCTGGGTATGGGCAAG
CCACGCCCTCTCAAGGCGGGGCCGAAGCGCCCGCCCTGCACTCCGCCTCCGGCTCTATAAAGTTCCTCTTTCTCACCTCACTTTCCTAGTTTCACCA
CTTGAACTTGGGACCCTTTGCTGCCCTCAGCTAAGAGTGCGGGTGAGGTAAGGCCTGTAGTCGGGCAGAAGGAGGAGTGTGAGGCTGGTGGCAGAGG
AAGCCTGGCTTCCATCTCTGAGCCTGAGGGAGAATGCTGAGATAGCGGACCCAGGCTCCGCTCATCTACGCTGCCCTAGGACCTGTGCACTTCGGGT
TTTGTATGAAGCTGTTTGGGTGGGAGTTCCAGAACATCCCCCACGGGCTGGGCGGGACGAGCTAATGGGACTGTGGTGTCATCAAAGGATCGCACTG
GCCACAGCTTGTCCTCAGAGGGACAGCCTCTGACTCTCTCTGCTCCAGTGGAAAGCTCCTTTCCAGCCCTGGTTCCTAAAGGACCCAAACTCATCTA
GGGCTCCAGAGCGTGATTCCTAGGCCGGGCAGCCAAGAAGAGCTGAGAGCTCCAAACTTAGGGTGCTCAGAGCCCCTTTCCCCGCATGCCCCTTCTT
CACTTCTCTGGCAAGAGTGCTAGTGTTGCTGTCCGCAGCACCCCTTATTCCCAGCCTCGGCTTCATTCCTGCCAGGGTTCACGCTGACATTCTGCAG
GTTGGAATCTCCTGTTTCTTGGCTGCGCTGCTTGCCCCATAACCAGACTTCCCTTGTTGCTTCCAGGACCCACGTGATGGTCTCTGGTTGGGTAGG
CCTGGGGTTATTCCGAGGACAAAGTAAGGGTGTCATAGAAGAAAGTCAAGAGAGTAAGCTAGGTACCGCAAACCTGCATGGCAGGGACACAGGACCT
GGACAAGGGCTAGTCCATGTGCCAGGTCCTTTTCGCCTGGGGCAGCCAGGGCAACCTAAACCCAGGAAGGGGCAAGTGTAGAAACAGTGAGGGAAAA
GTGGGATGAAAGCTACTTGGATCCAGCACAGAGGGACGAGTGAGCAAAGTGAGCGCCCCAGCGTGGCGCAAGACTTGGGATCTGCAGAGAAGCTGTG
TAGCTAGGAGCTTTCAACGGAGCGTGTTAATGTAAATGTAAATGAAGAAATTACCTAATTTTTTTAATAATAAAAGAAAGAACAGACAGGCAAAAAA
AGGAGGAGGAGGAGGAGGAGGAGGAGGATGGTGCGCGCCAAGGGATGCTCTCTATACCTTCGTCAAAGTACCTTCTCTTGGGGGACTTCGGAAGACT
CTGGCACTGCACCCGAGCACCTTGGCAGCCTCAGAGACTCGGGGCCTCGTGGGCACTGCAAGAGTTTGGGACGGGGCTTCCTCCCGCCTCCAAAGTG
ATGCGAAGGTAGTTGCAGGGAATGTGTGTCTCTCCTCAGCGCACAGCCCGAGGAGGTCCGCACGCGTCATGAACTTGGAGACGGGCAGCCGGGG
CTCAGAGTTCGGCATGAGCGCAGTGAGCTGCGGCAATGGGAAACTCCGACACTGGTTGATCGACCAGATCGACAGCGGCAAGTACCCCGGGCTGGTG
TGGGAGAACGAGGAGAAGAGCGTCTTCCGCATCCCGTGGAAACACGCGGGCAAGCAGGACTACAATCGTGAGGAGGACGCTGCCCTCTTCAAGGTTA
GCAGCATTCAGGGATCCCTGGGCAGGGGTGGGGGTGGGGATGGGGAATCTGAAAGCTCTGAATGTCTGTGGCTCCCGGGCAAGGGACTAAGAGGTGG
GCTCCTGCAAGGAGGAGGCCAGAGCATCAAGCATTGGACCCTGCTTAGGCAAAGTCCCCAGGAGAAGGGAAAGAGGTTGCAAACTCTCCGGGGATTG
CATCACAAGAAACCAGGTCCCAATACTGTTTGTGTGGAGGAAAGAACTTCCAGCTTCAGGGGCATCTCTGGGGGACCGAGGTTCCGTTTGCATAGC
CCATTCGCTGTTTCCTGCCACCACCACCGACTGCTAGGGCCACTCTCTGCTTCCCTGTCTCTCTGTGTTTTGTTATTTTTCTGAGTTTCTCTCTCTG
GGTTTTGTTTCTTTGATTGGGCACCTCTACTGTCTGGTTCTAGTTCTAGAAGCTGCGATCTCTGATTTTCTTTCTTTGAGTAGCTTTGACTATTCCG
AGTCTTTCTCTGGTATCCCCCTCCGACCCCGTGTGAGTCCCTTAGGACTGATGTCCCCAGAGAACTGGCTCACTGAACTGTGAAGCCCCCAGCCTCC
ACCTGCCAGCAGGCCGAGGAAGGGGACTTCCTGCGGGAATTTGTTCAAAGTACCTCTGTGATTTTGTAGATGTCCTCTCTGGGGCCTGCCCCCTCCA
CAGCTCTGTCCCCAGTCTTGCCCACATTGATTCAGGCGCTGGGCGTGTACAGCCCATACTAGGGGTCTCAGGACCCCACTAACATCATGTTCCACA
TTTCAGGCAACAGCAAATTTGAAACAGTAACCTTCCTTGCTGAAATGCAATCCATAGAATTCTTTTGACGCTGGGCTTGACTTTTCTTCTCATCG
TTCTTAGGCTTGGGCATTGTTTAAAGGCAAGTTCCGAGAAGGGATCGACAAGCCAGATCCTCCTACTTGGAAGACAAGATTACGATGTGCTCTGAAC
AAGAGCAATGACTTTGAGGAATTGGTCGAGAGGAGCCAGCTGGATATCTCTGACCCATACAAGGTGTACAGGATTGTTCCAGAGGGAGCCAAAAAAG
GTAAGGGGTTTTCCCAGCCCAGGTGGCAGGATAAAGGCATTATGGCACTCAGAGCCCTTCTTCCTAGAGACAGTCACGTCCTACCTCTGCTGTAG
GTTAAGCCCAGATGTCCTTTTGCCCATGTCCTCTCTGTTATAAGTGACAACCCTGTGGTGTTAGTATAGGATGACCTGGCAGACTTTAAGCCCCATG
GGTGTGTGGGTTATGCACTTGAAGGCATTATTTTCAGTTACTCCATTCAATTAGGATCTGGATCAAATTTCCAAACAAAATCTGGAAAATCCATTAA
ATGTTTACTTACCTAATATCCTCTAGTAAGCATTTTCAAGAGGAGAAAGCACATCCCACACCCCATACATATTCACACTTCTTGTAATAAAACTGCT
AGAGTTTCTGGTTTAACATGGCCTGCTAGGGTGGTTATGAATATTCAGATCTTGAGTTCCCTCTCTTCCAACTAGTCTACCTCAAGCAGTGCTCAGG
AATCTGCATTTGGTTCCAACCATACAGGATGCCTTAACTAGGTACCATCTCACAACCAGAAACCACTTGGTGGATCACAGGGATCCTGGGTGGTGTT
TCCTTCCCTGGCTGTCACTCACAAGTCAGCAAATGTTTAATCAGTTTAATGGCAAAGACAAATATCTCTCTAAGAAATTGCTTGAAAAACAAACAAA
CAAACAAAACAAAACAAACCTAAAATACCCGATTGGTTAATAGGGCTATGCATTCTAAGAATTAAGTGCATAGGTACTTTTATAAGATTTAAGTCAG
TTCCTTGTCTTACTCTGTGTTCTCTCTTCCTTTTCCCCAAACACACAGGAGCAAAGCAGCTCACTTTGGATGACACACAGATGGCCATGGGCCACCC
CTACCCCATGACAGCACCTTATGGCTCTCTOCCAGCCCAGGTATGTGGTAGACTCTTGGTCTTGTGGAAGGCTGGCCCATGCCCTTTTGACTGGCTC
CACACAGAGAGGCAAACACAAATGAAAAGTGTAGGGCTGACTTCTTATTTGCTATGGCTAGTACACACGCTGAACAAAAACTTGGTCAGAGAAGGAT
GTTTCAGTTCCAGTGTGGTGTCACTGTCCCTGACGCCACAGTTTTGTTGGGGAGTTTGATGTGTCCCACCTGTGGAGAGAGGCTTCCACTGATGGTC
AGATCTTCTGGGAATCAGACCTTTTGTGGAAGTCAAAGGTTTTGGAAGTAGTACTTTATCATGTGAAACCGCAGAGCAGCTGACTTCTCTAGGCGTC
CCTGATGTGAATTACAGTACTGTTTTATTCACTTTGGTGGCTTAAAAAGGGCAGATTTCACTGCGGTATTCTTGGTGCCGTGTTCAGCCATATGATG
AAGCCTTACAAAAATCACAGCTTTATACAATGTCCTCATTGTGCTTTCAGACCCTCTATGGCTGTTTTTTACCTAGTGTGATAGACAGTCCATGTCA
CTTTTTGGGCAAAATGACTTGGCTGCTGGACAAAAAAAGGGGTTCCCTGAGGAGTTTGGGTGATATGAAATGACTCCGACACCCACTGATGTCTTCC
TCTTAGCAATCCCTGTGCTCTGTCAGCAGGTTCATAACTACATGATGCCACCCCATGACAGGAGCTGGAGGGATTATGCCCCTGACCAGTCACACCC
AGAAATCCCATATCAATGTCCTGTGACGTTTGGCCCACGAGGCCACCACTGGCAAGGCCCATCTTGTGAAAATGGTAAGGATTGTGCCAGGGCAGCA
GACAGAAGAACAACCTGAGCTCGGGGTGTGGACAGCACCACAGGGCTTTTCCCTACCATTGAGATACCAGAGACACATCATATGAAGCTGCTACTGT
TGTTGTTGTTGTTGTTGCTGCTGCTGCTGCTGGGGTGGTGGGGTGGTGGGGTGGTGGGGTGQTGGAGTGGTGGTGGTGGTGGTGGTTGTGGGGTGTT
GGGGTATGTTGCCTTGTCCTGTGAAATGTTGAAGTCCTTAGATCCATGATAGGCCTCAGTCTGTGTGGGGACTTAACTAGAAGACCCCAGAGATCAT
TCCAAGTAGCTGAAAAGTGCCCCATTTTTAATACATAGAGAAAAACATGGATGACAACAAATTCTCAATGACAGTAATGTCAATTATAAAACTCGTC
TATATTTTGTTTTAACTGAGTTATCCCTTATTTCCGATGGTGATTAAGTTGGGGGGTTTGTTGTATCCCACCTATCTCCCTAGTCTGTATCTTTCTA
CTCTCCTGTAAAGTAGAGAGTTGTACCCAGTCCACCTCAGCAGGAAATCATTGCTAGTTCATGTCTCTTGAATAATAATGAGTCATCTATAGCTGTT
CTTGGTACTAAGGAAGGAAGGATCAGAGCGAAAGTAATCCACAAAGTGTCTCTACAAATGAGTGCCCTGCCCGAAAGACCCACAGGGGTCCCCCCAT
GCTAGCTGGGCTCTCACAGAAGAAACGCCCACTAACCAGACACAAAAAAATTTCACAAACTATGTTCAGTGAGACTTGGGTCCTTTAGTGTTTATTT
AGGTGAGTGCACCAAGCTCCACCTCGGGTCCTTTTTTGGCTGTGTATTTTAAGGTAGAGTCTTGCTAAATTACCAAGGCTAGGATCTTCCTGCCTTC
AACTCTTGAGTAGCTGGGACTACAATCTTGTTCTAACGGGCTGAACATAAAACAAGTTTTTAGGACTTACAAGTTCACTGTTTAAATATAAGTCTTG
ACATGGGTCGCCGTGCGAGTAGTTCTTTTATATTGTTCTGGCAATACTTTCCTTGTGACAATTTCATCAACACCCTCACTCAGTCTGTGCATGCTTA
CACTAATCTTGCTTTAGTGTGACATAACTTCTCTGCTGCCAGAGAACACGGTTCAGCCCCTCCCCCTAGCTAACAAACAGTGAGCAGAATAAATGAG
GGTTGAATAATTAATTCATCTTTGAACTAGTCTTATAGAAGTTTGAACTCTGACCCTGCTGGTAACTTGCTATGTGGGCTGGTGCAAGTCCCTCTCC
TTCTGGGCCTCAGTTTCCCTATAGATTTGGAGTGAGCCCCAGGTTTCCATCCAGAGCTGTACTGTGGCTCCTTCCTTCATCACCCTAATTTTTATCA
CTGGATGTGGACTTTGGACTTTGTCCCATAATCACACGTTATGCTGCTAGCAGGTGCTTAGAGGCTGTCAGGCTTGGGTTGGAGGCCATGGCCTCTC
CCAACTCAAGAGCCTCCCCGCACTCAGACTCGATACTTAGACATCATCTGATTTTTATTTGCAAATGCAGGTTGCCAGGTGACAGGAACCTTTTATG
CTTGTGCCCCACCTGAGTCCCAGGCTCCTGGAATCCCCATTGAGCCAAGCATAAGGTCTGCTGAAGCCTTGGCGCTCTCAGGTGAGTGTGGCGCTTC
CTGTAAAGCTCCGAGGGAGGGGGCATCTCTCCTCTACTGAGGTTGGGTGAGGATTTAGACTCTCGCCTTGCAGGCCCCGGGGTCTGGAGTAGGCATG
GTCCAGGCTATGTGGACATCACGCTGAGTCAAATACACTATTAGAAATCTCCACAGCAGTACCAGCTAGCCAAATACTATTTGGACGATGTCTTTAA
CCTTCTACATCATTACCTGCCCAGTTTTCCAGGAATGTGTAACCAGGCTCCTCCTCCAGCCGACATTCTCCATTCTCGCAGTGTGGAAAGGCTTTAT
AGGCACAAAAGAATGCTGTTTGTCCTTTTAGGGTGTAGGGTTGGCCACAAACAGGTGGTCTGAGTTGCTTCCAAGGAACACTGGTTCTGAACCCTGG
TCTCTGAGAAGTTCTTATGCCCCCTAAAGGATCATATAGGTCTGACTCCCTCACAACTTTGACAGAATTGCTGAGCATGTGTGGATGTGATCTGATT
TTAAAGTTCTGTTACTAAGGAAGCCTGCACTTGGAGATACTGACCAGCATTTTAAAAGCCCACACTCCGTGGAAGCAGACATCTTATGTCCATTTAG
TCTTTAGATGATTTTTTTGGATGTTTTCAAATGGAATTATTAGAATTCTCATCATGCCCTCGGCTACCTTAAAAGCCTCTGACTGAAAACATCAACT
GCATTTTGACAATTTTAGACACTTCCCTTGTTCTCGAGGGAGGAAGAAGTTTTAAAATCTAGTTCCTTCCAGCTCTGATGCTCAGGGAGACTTTGTG
AGCCACTCAAGAACAGCCGAGGAGCACATCTGGGCATCAGGGTTTGTCACAGACACTAGAATGCTCTAGATCCTCTTCTGGAGCGCCAAAGACTTGT
GTGGGTGCCCCAAGAGTAGGAAATAAACAGCTATTTATATCTCTGCAATCTTGTGATTTTGGTGACATTAAATGAAATGAAACCTGCCCTACCACTC
ACCTCAGATGGCCAACGCCCCCTCTCTTTGGGTGCACCACTTGTGCTGTTCATAGCTGCAGCTATCGAAGAcAcCATGATGTGGGcTGTCAGAACTT
GCCATTGAAGAATACGAGGCTTTTGTGGGTTTCTTCTTCTAGTTTGCATAATTAATTATCAACCCTGAGTGCACTTTTCAGAAAGCTATTCTTTCCA
GGCATTGTTGGGGCTCCAACCACCAGCACGGGTATCTATCTCTGCCTGGGGAGCCCTTTGCACACCCAGCTTGCCCTTTCGGCCCGTGGGTGGTATT
TTAAAGTGGCTTCTGAAATCAACAAAATCATGTGTCAATAAATTCCTGTCTTAAAGCTGTAGAAAACCTAGTTGTTGGGTTCTTTTCAGAGTTGAAC
ACGAAGCTTAGAGGGATTTCAGGGGGTTTTACATTAACCACTGGCTTTTAGAGCAGTTCTCATCAATTTCTTCCCCTACTCCAAGAGAGCTGACTTA
AAAATAAGAAAATAAAGGTATCATTTTCCAGAGCCCAGAAATTGTTATTTTAGTGCCTGTCTCTAACATATCTATGTGGGTTTTGTTGTTGTTTGGT
TTTACTTAATGACATCATGGTAACACCTTAGGGAAGTTCCAGAGCTGAGGACACTATTTGCTTTTCTTCTAAGATGTTTCTGTATTTCTTTTACTAA
TAGAAATCTGTCCCAGAGGTCAACTCCAAAATCAAAATTGAGTTGCTGGAAAACGAATTCCAATTCGGTAGTATTATTTCATATTGTAGACAAAATG
CCACCACTGTTAACACCATCATCCGAAAAGCCCTCATAACAGGGGTGTGCTTTCTAATAAAATTTGGCTGAAAATTCAAGAAATATATACCTCTCCC
CAAGAGAAGTAAATGGCCACAACAACATTTGAAAATGATCGTGTTAGAGAGATCAGTTTCTTTCCACAAGCTTCTCTTAGTATTCTGTGCTTGAGGT
CTAAGAATCTACAGGGAATAAGAGCAGCTAACATCTCCAAGACTTCCTTGGTCCTAGGATCTTTCACTTGTTCGTGGAGCATCTTGACACTCAAGTG
TTCCACCTGCTGTCCTTCGTATCAGTCTAGTCACCGAGTTTTTGGGGCTCTGAGCAAGGTGGCACCTTTTTCAAATCCATCAGCACTGACTCCAGAG
TTTTGTTCACAGACTGCCGGCTGCATATCTGCCTGTATTACCGGGACATCCTCGTGAAAGAGCTGACCACGACGAGCCCTGAAGGCTGCCGGATCTC
CCACGGACACACCTATGATGTTAGCAACCTGGACCAGGTCCTGTTTCCCTACCCGGACGACAATGGACAGAGGAAGAACATTGAGAAGTTGCTGAGC
CACCTGGAGAGGGGACTGGTCCTCTGGATGGCTCCAGATGGGCTTTATGCCAAAAGACTCTGCCAGAGTAGGATCTACTGGGATGGGCCCCTGGCAC
TGTGCAGCGATCGGCCCAACAAGCTAGAAAGAGACCAGACTTGCAAGCTCTTTGACACACAGCAGTTTCTATCAGGTAACACACCTCACAGTCTGTT
AGAATGGAGGTGGTGGTGGGTGCTGGCTATAAAGGTCTCAAATGGCAGTGTCTGCCTACCCCAGACAGAGGTCTTCCTCCTGAGATCTGTGAGCTCA
TGCAGAAATAGAATTCCTGCCTGATTCATGCCTAGCCTTTGTCTGTTGTGTACTCCCCTGATTAGCAGAGGGCCAGAAAGAGGATCCATATTTGCTG
GCCAGGATAGACACTGGTGTGGGTTGATCTCTTAATTTATCATCATTCTTTTCACTCTAGGCTTTTGTTTTGTTTGTTTTGTCAGAATATATGTAGC
TCAGGCTGGCCTAGAACTCCTGCCTCGGGATTTTATCTGTACACCAGCACATCTGGCCAATGAATTAAAATGTGGGCTTTCAGCGGCATGTGCCCCA
CCCCCAGAGAGGTTTCACTGTGTTGGCTCTCTGCTCTCAGCAAGTTTATCTGCTGACACCTCAGCTCTTTAGGGGTTTCTAGAAAAATCAAGAAGGT
TAGAAACACCATCTTAACATTTTTGGTTATGAGATGTGTCACCCTTATAAAATTGTCACACTTGCGAGGCAGGACCAAAGCTGAGAATGTAAGGAGC
AGATTCAGCGAGGGGGAAATTCGGAGAAGAATTTGGTCTTATCTCTGGCATACGAAGGCTTCTAATCATATTTTCCAGAACCTGTACTTCTTATGGT
ATGATTTTTGACTTTGGGTAGTTACGTTAGACCAGGGCTGGCTTATATGAAGTCATTAGTCTTGCCACTGGTGGGTACCACCCAGAGGCCTCCCCTA
GAAACGTAGCCGAGGATGCCTTTAAGGAAGCAGGCTGCTCTGCTGACTGCTAATTGTCATTCCCTCACCCCATCATATCCTAAGCTTGCTTTCTTCC
CAATGCCCTTCCCAAGCAGCTCAGCTCTGGCTTTCCTGTGCTTTCCTTTTCTTGAACCGGGACTTGCTCCTGCTCCATCTCATCCATGCACACTGT
GATGCTTTAAAATGCGTGCACACTAGGATCTGGCTAAATCCACTAATTAGCATGCATATCACCTTGCCTGTCTATCTTTTTTTAATTTGGTAAGAAC
ATTACTTGAAATCTACTCTCGATATTTTTTTAAGCACATGGTCCATTCATTATTAGTCATGAGGATTGGTCCTTGAGCTTGTGTTCAGCAAACTCCT
CATGTCTTAGTGACAGTTTATATTCTTTGACCCACAGCTTCCTAGCCACTTCCCACGTGTGGTGACCGCCATTTGGCGCTACTTTTATGAGCTCAAC
TATTTTGGAGTTCGCAATGCAATGGAGCGTCACGGTCTTTGTCTTTGTGTGCATGGGTAATTTCACTTGGCATGATGTTCTCCAGGCTCATTTGTGT
CATTACTAATGACAGGCCTTCCCTCTTTATAAAGGCTAGGGATATTCCACTGGGGTATCTGTATCGTGTCACTTTCTCTTTATCTGTGTATCACTGT
GGGTACTTGGGATGATGCCTTGTCTAAGCAGGTCTGACGGGTAACGCTGAGTGGGGGAGTATAGCTGTCTCTCCAAAACCAATTTGCTCTGCCTTGG
TTACGTTAGACCAGGGCTGGCTTCTATGAAGTCATTAGCCTTGGGTCTGCACACAGAAGGGGACTGCTGAGTCCTCAATGTGCTGCTTCCTTCTGTT
CATTTACAAAGAAGACGACAAGCCCTAGTGTGCTTGGTACTAGTGGTGTTTCCTCAGCCTTTTGAGCTCCAGTGGCCAATATTTGTTTAGGCTCATC
CAAATCCTCTTTCGTTTCCAAAAACAAACATGCTCCTTGCTAAATATCCTCCOCTGAGAGGTGGGACACAGAAAAGAGAATCATCTCAGGCTCCAAG
GTGTCTCAGGCTCCAAGGCGTCTCTGGCTCCAAGGACTGATGAAGATCCTTGGAGCTGGGTGTGATCTAAGCCTTGGTCAGACAGCCTTTGCCTCTC
TTAGAGTGAGTTCTAGTGTTTGTGGTCCAAGGCAAAGAAAAATAACTTCTGGAGATGGATAGGTACACACATCCTATATCTGCCCTGGGGTCTGGAA
GTCCTATGTGCCCTCCAGAAGGCTACCACTCACTCCCTGCTTGATTAAGGTAGCTCAGAATAGTGACAATTGCCAGCGACAGGGCTTTGAGGTACAC
TAAGTCTTGGTGTGCCAAAGAGGATCCAATTTGGCAGGAAACAAATGCCAAAACAGAGCAGACACCTTGGACTAGAAATTTGCTTCCTAGGACTTAT
TAGTTATTAATGAGTAAGGGCTATTTCCCTTTGTTCCCTTCTTCTCTAGTGGGGCCTGTTGTTTTTGCTGTCCACAGTCCCCAAGCCCCACAGAGGC
TCAGGGAGATGACATTGCTGCAGAQAGGCCCAACAGCCTCAGAACCTAAGGATGCCCACCCAATGGTCCGGGAAGAGCAGGCTGAGGTGGGCTTTC
TCAGAGAGTCGCATCCCTTGCTATTCTGACCTAGCCCCTCGTAGGTGTCTTCCCGGCCACAGTGCTATGCACTTTATACAGTGTGCCTAAACCCCTG
GGTTTGATTTAAATTCTGGAAGAAGAAAGAGAAAGAAATGCAGGAAGTCAGTTCTGTGAGCTTCTAGAAGCAGTTCGGTTGCAGAGAGCAGTGGAAA
TCTTTGATGTCTACCCATTCTGGATTTGCACCCCACTAGGGACAGTCCCCATAGGCACAGTTGAGAATTCATATCTGATCAGGGCAGAGTCTTCATG
CCTGCTCTGTGGAGGCAGCTTTTTAATGTCAGTTCTTTGATGCAGACAAGACCTGGGAACCTAGCTCTGGGAGGAGGAATAAAGGTTAATGCCAGTG
AGTGGATGTGGCTTTCTGCTTGTGCTGGGGGAGGAAGCCAAGGCCTTGCACATACAAGGCAAGTGCTCTGCTCCAAGTGGCGATGCCCCCAGCCATG
GGCAGGTTTCTTTTCAGCAATCTTGTCTGTTTCATGTCTCTCAGGcAGGACTAGCCTCAGCATGACATCCTTGTCAGAGGGGCTTCATTGGTCCCCT
TCTCCCTGTATCATCCTGTCCCCAAAGTGAGATTGAAGCCTACTCTGGTTCTCCAGTTTTGGAGTTTTAGACCTAGTGCCAAGTAGGACACAGCTGC
CAACAGCTGGTGAGAGAAACAGATGCTCTTGGTGCCCAGACACCACGTGGCCTCCATGGTTAGCTAGTGAGGTTAAAAAAATAACCCTGGGCCATCA
GAACATTGTGACTCTTTACATTAAAATGTCTCCTTGGCCTGTGCTGATTGCTTGACTCAGCATGGCTACTTTTCTTTTTCTTCTTTGTCTTCTTCTC
TTTGACCTTGTGCATTTCTGTGAGTGTAGTGCTGCAGACCCAAGTTCTTAAGGTTGGGTCATGTTCCTTAAGAGTAATGAAGTAAAACCAGTGCCAA
GTCAGGAGATCATATGTGAACTTGACCATGTGATTTTGTGTCTAGGGTCTGCTCTAAGGGCTGGACTTAGGGGAACAGAGCCCGGGCTCTCCCAAAG
CAGACTTCCACGTGACTCTGGCTTTCCGTTCACCCGCTTTACCAGGTGTCTGAACAGTTTGGTTTTTTTTTTTCTTTCTTTCTTGTGGGTTTTCAGA
GCTGCAAGTGTTTGCTCACCATGGCCGGCCAGCACCGAGATTCCAGGTGACTCTGTGCTTTGGTGAGGAGTTTCCAGACCCTCAGAGACAGAGGAAG
CTCATCACAGCTCATGTGAGTACCTGGTTACATCACCCGTAAATCACACACTGTGGAGCTGTCCCTTTTAGAGAAGTGGCAAGTGACGAGTAAATGT
CAGCTCACCTGGGAAAATAGATGTAGACCTTAAAATAGTGCAGGAGGAAGCAGGCTCCAGTGAACACCACAGCTCAGGGAGGCACCCGCAACCTACT
TCCAGACAAATTCTGTCACCACCGAATCAGCAGGGCAGATGACTTGGACCCAAGGCTCTGTTTGTTCTGTATTCTTTATTGTTTCATACAGACAGTT
ACCTGCCCTTTTATAGGAATTTTCAATAGTTGGGACCAAGTACTGCCCTTCGACATCTCTGTTTCTTGTGTGGTTTTAAAGATGCTGTCCTTTCGAG
TAGAGTAGCACTTTCTCCCTGGGAGGCTGCCTGTTATGTATTATGCTTCATCGTGCCTCCTAACTTCAGATAGTTCCCAGACCCTCGTTTTGTTGCT
GGACTTTAGGGAGTTATTTAACAGTTGGACAAGGGAGGTGGAGGAGGCTGAGTCTTCCCAGGAATCAGGTAGGTCGGTCTATCCTCACAGCTAGGGT
TTATTCGGATAATGTTCATCACTCACTTAATAATTAAAAGGTAATTCTGAATACATGATGTTTTTTAATTAGAAAATTTTACTTAATTACATATCTT
GAAAAGTATGCAGTGTGGAGTAAAGGTTGTGTCCCAGATAGCCACAATATCTCAGTGCAAATGGGATATTAGCTCTGATGATATCTCTTAGTGGAGA
CTGAAGACTAGGCATACAGCGCAATGGAAGGCATTTGCTAGGCAGTGGTAAAGCCCTGGGTTCTAAACCCCGCCTAGGATGGGGGTTGGGCACTGAT
GTTGAACATCCAGCCTCCCTTCTCGGTTGCAAAAAGTAAAATCTAAGAAGCAACAAACGGGCTGGAGAGATGGCTCAGTTGTTAAGAGCACAGGCTG
TTCTTCCAGAGGTCCTGAGTTTAATTCCTAGAAACCACATGTGCCTTACAACCATCTGCAGTGAGCTCTAATGCCATCTTCTGGTGTGTTTGAAGAC
TGCTACAGTGAACTCACATACATATAAATCTTAAAAAAATAAAAGGCAATGAAACTATGATCCTGGCCTTGAGCCTTTTCTCAGTTCTAACTGGTGG
TTGATATCAAATGAGACTGCAGATGTGTGGATGAATCTAGCATAGATAAGCAGTATTTTTTTTTTTAAGGThGTGAGTAAATTCTAGCATAGATCTC
ATTTTAAGGACTTTGGGTGCAGTGGGGCTCCGCAAAAAGGGAGCAACAATAGTCATATAGGCAAAGGGCCTCAAAATGCTGCCCCGTGGTCCACAGA
TGGAAAACATACATGGTCACCCATGAACTCTGCTGGTCTCCTTATTACAGACTTAATTCATATGGGTGCTTACAGAGGAATCCTACCAGACATCACA
TATCAAATAACAAAGAGGCTTGATTTATTGATGATTGGTTGTTACAGAGCACACAGCCTGACTTGGTGAGGCTGGCTTTGACTGGGGATGCAATCGA
TGCTTATAAACAAACTAGGTCCATCAGAGCCAGCGAGCTGCTGTCTTGTGGCTGGCCAGCTCTGTCTTCTACTTGTGGTTCAGAGTTCTGTCTATTT
CACAGTCATCTGGTTCTTCAGGATGAGCCCTTCTGTCAGACTCATGAGCCTCACTTACOCAGCATGTTACTTAGCCTTTTAATTTGGTCATCTCATT
CAATAATGTCCAGTTAACTCATTCGCTAAATATCAAATCCAAGAGGTGATTGGTTTCAAAATGCCATATTTATCTTCTATTATAGAATCAAGAGTTC
TTTTTCCAGGGTTTTTAATTCCAGGTATTATAAGAGCAAATGAAACTGGTTTTTCAAATGGCTCTGAATGTGAACTGCTTCACTGTGTTATGTTATC
CTGTGCAGCTTGTAGGTTTTTACTTAGAGTCCTAGGGTCATTTCATGATGTCCCAATTGTATGGTGTTGAGAAGAATATTCTAGTGATGTCTTTTTT
TCTTAAATGTCTTATTAAAGGTGGAACCTCTGCTAGCCAGACAACTGTATTACTTTGCTCAACAAAACACTGGACATTTCCTGAGGGGCTACGAGTT
ACCTGAACACGTTACcACTCCAGATTACCACCGCTCCCTCCGTCATTCTTCCATCCAAGAGTGAGAAGAAATACTCTGACAGGGCAGCCGGCTGCTG
CCCTTTCTCTTTGGAAGAGCTAAGAAGTGAGTGGGTTTCCACTTGAAGACAACAACAGGGCTTTGTGAGGAAAAACAGCTGTATCTGCTCAACAGAG
GAGCTTCCCCCAGAAGAGTGCCTGTCATCCAGGTCTTGACAAGTGCCAGGACTTGGGTGACTGTGCCCTGGCTTATAACTGTGAAACTTGATCAGTG
TTGTGTTTACATGTACTTGAATGCTGGCTTTAGCCTGGTATAGATGGACTTTTGCTTGAAGACTGAAAACCTGTGCCAGCATGAATCCCTGACAAGA
GAAGACATACGTATTATTGGTCCATTTCTCAGGGAAGTAAAGTCTAGACTAGAGACTACGCTGTCCACTCACAGAGAAACGCATTCCTGGATAGAGG
AGCTGTAGGTTGCCCAGATCATGTCCACTGAGTGAAGGGACTTGGTTCTTAGGTCCAGTCTAGGCTATCATGTCCTGATCCTGCACTCTCTCTGAGT
AGACCCTACATCCTGTTTGCTCCCTCTTCTTGCCCTTCCCTAAAAGCCAGCTGGTGGTGGACTCTTGTTGCCACATACTTGAGCCAAAAACCATCAGGG
ACATATAAGATGAGACATGTTTGCATCTCCTCTGATCTAACAACGGCTACAGTTTTTTGTTGTTGTTGTCTCCCCTTTCTGTTTTGCCTCTCATT
CTTTAGAATTGTTGCTTTTTTGTAAAGAAAGGAAGAGAGAGAGACCGAATATACTAGAGATAGATTTTTAAAATTCTGTGCCATTTCCCCTTCTATT
TGTTTTGTTGGCCATCAGTGGTCCCCATGGCAACACTGGAAGGGCGGAGCCTTTGAGCAGGTTGGGAACTGTCCCGGACACCAGTGGTTCAGTGTAC
AGCTGTAGACTCAGTGAGCTGTTTCTGCTTATTTTGTATATTAGATGCTTCCTTGTGCCAATAATAGTTTGACAGAGTCTCAAAGCTACCGGGCTTC
TTTTCCAACAACACATTCTTTCACAGAATCCCAAAGAGTCTCACTTGCCTTCACAACCGTCTGATCCTTTATTGTTACTGTTTTGTTTAGGTGGCTT
TGCCATGCTCTGGGATTTTTCTCCTACCACAACATCATTCCTGACTCTTTCGGGCTCAGAAAGAGTTAAGCGCTCTTGCACACTTGGCACTTACTTC
TTTTTAATTTTTTTTTTAACATGCCACTCTTAATAATATTTCTTCTTAAGTCTTTCAGCAGCATACATTCAATAAGCAATGGACCATTGGGTGTCTCATT
TCATTGTTTTATTTTTAAAAAGGAACCTTGACAAATAATACACATTACAGAAAATGGAAGCATTTTTCATAGAGAAGGTAAGAACTGAAATGCATT
TTATGTTCGAATCCTCCTTGCTAGTATTAGCCCCAAGAGTTCAGTCTGTCCTTTAAGCATGTTTTAGTTTTCAATACATATATATGAAGTTGCCCTT
TCTTAGAGAGAGAGAGAGAGATATGTGATGACACTCTTATGTACCCAACTATACAGATTTACCTCATCTTTTGAGTGGCATGTGACTTCCACTGCT
ATGTGCCTAGGTGCAGTTTAGACATCTTCTTAGCTACCCATCCCAGCAGCCCACTGAGTCTCGTGAACTAAAGTCACTTATACATCTTATAGATGCA
AAACATTCTGGGGTATATTATCCCAAGGGAAGATGAAGGCAAAATCGGGAAGCACTCTTCCTGAGGCCCCAAGCCACAGCTGTCCTCCTGGAGTATC
TTAACCATATCTGGCACTTCACCAAGGTCCCTTGCCTGGTCCTGTATGACCATGACTCTTCAGTCCATAAGCAGATGCTTTGTCTCAATTGCCATGT
GAAGTCTTTTGGTTTTTGGTTTTTTTCCTTAATGTTAAGCTGTTTTAACAGTGAAGCTGAATTTTCTGGAAAATGCTGTATGGTTCGAGCCCCCAGT
TCATGTTTCACTTTACAGCCATGCATGCTGATGCCTTCCATCTTGACAAATGGAATGATGGGTAAACACTCCTGACACAAAGAGGAACACCTTCCTC
GTCCCTTGCTGAAACAAAGTGCAATGCACACAGTAGAATTTAGTTTGCTTGCACTGGAAGATGCCGTTGAAGAGGTAGGCTGAGGAACACTGTATCC
TAGGGAGAAAATATGCAAGTCTCACGGGTGCTTTCTGTTGGCTTGTTTCTGGTCCTTGAGCAGGCTTAAGTATCTAGTTTCAAACTAGAAGCCCCAA
AGCCCTCAGTCGTTGTCCTGGACAGAGTTGGTAGACCCCACCTGTAGAAATCCAGATGCACTCTGGGATTGGAGCACAGAGAGGCTTGCTCTATCGT
GAGAATGCCCAGCCTGCAGAGGCCCAGGTGCGGTGCCTGAGTGGCTGTATGCCAGACACAGGAACGCCAGCAGGCTGAGTGTGGTGACTGATATGTG
AGGTGCCCTTGCCCTGCAGACAGGCTGAATGTGAGCCCCAGCAGCAGAGGATTCTGAGAGTCAGGGTGGTGTCATQTCCTACACAGGAAGAGCCTTC
AATATTACTTTTTAAACGAACTTTCCCCATTGCTGATATTTTGATAAAAACCCACACAAGGTTTAATTACATGCCCCCTCATCCCTCTCCACACAGT
CTTGCCCATCCCACTGGTGAATTGTTGTTGTTTTTTGTTGTTGTTTTTTGTTATTGGTGAAAATTGTGAAATTGTTAAGGTCTCTGTTTTTGTTATTT
ATCTCCCTAAATCACATGGCTGCTGCATACATTGCAATGTTGCCCTACCACATGTATGTTTTTGCAAGTTGCTTCTTCACTCATTGAAGAAGCCTGC
ACCGTAAGTGAAACTCACTTAGTCCTTGCTAATTGCTGCTCACTGTCACGTGAACGCAGAGAAGGCTGATTTTCCTGGACCCATTTTCAGTGCTTTG
AGGAACATGAGTCCAGAGTATATTGAAGTAGAAGACAGTGTTTGGCAAGCCAGTTCTTATTAGACACTGCTACAGGCTTCACAATCTTCAAGGTGGA
CCCACCTCATGTGGAAGGGCATCATTGCCCCTCTGCTCCCATGCGCGCTGTGCACCCCCTTCACAGTGAAAGACCTGCTGGGGACCAGGACTCAGAGAT
CTCTGGCTTCGAGCTCATATCTTGGCTTCCTGTCATGTCTAAGCCCGACAGGAAAGTGTGGCACCATCCTGCCTGCGGCGTTGGATGGATAGGCCAT
GGATTTTGCTGTAAACTTGCTTCACAAGGAAAGCTGTGTGTTTTAAAATATTTGATAAATAAAGGATTTCTAGAAAGATTGACTGAGGAGCATATTT
AATACCATCCTCACAGCCCTTGAAACACTTCCTCTATCTGCTGGGTTCTTGGTGAGGCTGTCAGTGGCCTACTTCCTTCCTGGCTGCCTGTGAGTCC
CTGAAATCAGATCTCTAAAGTGTTCAGCAGAGAGAGAGAGAOAGAGAGAGAGAGAGAGAGAGAGAGAGAGAGAGAGGGAGGTTCTAACTCATGGCTC
TCACCATTCCTTCCAGCATACTTTTCTCAGATCACTATCCCCCACTGTTCTGGCTGTTTTGATCCTTAGTCCCAAAGGAGAGGAGGCAAAGCAAGAG
AAAGTATGAAATCGTTTCTCAGTGGCACAGAGATGCCACTGAAAGGTGAAGTGCTTAGAAAGAAAGGATGTTCTATCATCATGAGAGAGAGCTGGCC
AGGTGAGCAGGTGGGCAGAAGTCTTCCAGGGAAGAGCAGGGTACCTGTGTAGACATCNNNNNNNNNNNNNNNNNNNNCCTATGTTCTGAAAGGCCTG
GTAGACTTGGTGTTTTTTTTTTTTTCTGTTTGTTGTGTTTTTTTTTCATTTTTAGAAAAAAAATTTAATATCTAGTGTGTGAGTTTACCTACTTGTA
GGTACATGCACATGCAGTACTTGTAGGGCCAGGGAGGGGGGTCTGATCCCCTGTAACTGTAGGTACAGGTGGTTGTTAGCCACATACGCGAGAACCT
GGAACTCCAGTCCTCTGGAAGAGCAACAAGGACTATTATCCAAGTTGAGCCATCTCTCCAATTAGGGAAAATATTTTTTAACTTGTTTTAATGGTTT
GTTTTATACTTTGATTTCCTCTGAGTTTGTTGTCATGAGGGTAGCATATAGTGAGCCTGCACTCTGATAGGAGAGATTCCGGGGGAGAGGCACAGGG
TGTCGGTAGACTCTTGGGTATCCCTAGGAGAACCTTGGCTGAAATTCCTCTCTGAGAAGCCAAGTCAGAGAATGCCGTTCTCAGCTGTATCACACAC
ACTTCCTGACATGGCTGTGAACTTCCGTATCTCTTCCAGTGTCATAGGAAGACTGTCAGCCCAGGATAGGACTGTGGCCTGTGAAAAGCTCAGAGATTC
AGAGATCTGAGAAAAGCATTTAGCAAGGATCTAAACAACATATTGAATTTACTAAATAAAAACCAATTGCTCTACTATTCCTACTTTGGGAGCTCTG
TGTGTCCCATAAGTATACATGGGCTCAGGCCAGCATGCACAGCCAGGATTTCTCCAGAAAGTTCTACTACTTGAACCTAAGCTTGTTTGAAGAGATT
TTGCTGACTTTTGAGTTCTTACATGATATTCCTATTCCCACAGGGCTTCTGGTCTTAGACTTTTCTTTCCAGTTACAACTTTGGTTGTTGGATTTGG
TTTTCTATAGTTGGTAGTATTAAGACAAATACAGGGTTTTTCACATGGGCAACACTCATCCATCCCAGAAACAAACAAACAAACAAACAAACAAAAA
CATGGCGTCTCCTACATGCCTTATTTCTGGGAAGGGACTGCAAGGTTGGTTGTAATATCTCATGTTTTCTAGCAGAAATGTCTTTTAATAAATGTCT
AGTTAGGAATTATAGGGCACCCAAGGATGTTTCTTCTACAGCCTAAGCCAGGGGCCAGAGGATGTCTACACAGGTACCCTGCTCTTCCCTGGAAGAC
TTCTGCCCACCTGCTCACCTGGCCAGCTCTCTCTCATGATGATAGAACATCCTTTCTTTCTAAGCACTTCACCTTTCAGTGGCATCTCTGTGCCACT
GAGAAACGATTTCATACTTTCTCTTGCTTTGCCTCCTCTCCTTTGGGACTAAGGATCAAAACAGCCAGAACAGTGGGGGATAGTGATCTGAGAAAAG
TATGCTGGAAGGAATGGTGAGAGCCATGAGTTAGAAAGAACCATAGCTGCAAGATGAGAGAACAACTCCATGGTTAACGGTGTGGATCAACACCTGA
AAATGATGGAGATGCTGAGATGGGTCTGATCAGAGCTGACTGTGGGCCAGAACTTACACACACACACACACACACACACACACACACACACGTGCGA
GAACCCCTCACACACACAGGATGGTGGGGCAGAGTTTAGGTACTAGAGCTCCTTCAAGGGTGCATCTTCAThATAATCTTCAATTATCTAACTTT
TCTCACACTAGACCCCACCTATCAAAGGTTGTACTTAAGGTTGTATCACCTCCCAGTAGTGCTCATCAGCCACAAAAGCThTTAACATTGGTATTTA
CTGTTTGTGTCTATGGCATATCACTGGTCATTCCTTTTTTATTTTTTTAACTTTTTATTGATTGTTTATGAATTTCACATCATGTACCTCAGTCCCA
CTCATCTCCCATCTCTTCATATCCACCCTCCACCCTTGTCACCTACCCGCCTCCGAAAAAAAATTTTTGGAAGCTGCAGTGTGTCACAGTGTGTCCC
ACAGTATACCCTTTTGTCCACACTTCCTTATATGCAAATGTTCATTGCAATGAGTCATTGGTCTGGTTCAAGGCCTCTGGCTTCTGTTACACTGTCA
ATACTGGATCCTCACCAGGGCTCTTCTCATATAGAATCTCCTCCTGTTGTCCTATGTCATGGAGATCTTGAAGCTTTGGGCCTGTAGTACCCTCTCC
TTCACATATACCAGCAGTTCATAGATGGCATAGAGCTAACTGAAAACCCTGGATCTGGGCCTGGGTGGTAGCTAAGTTGGTCAGCATGCCAGCTCTC
CTGCACCCACACCACCAGGGCAAGCTCTCCAGGAGTACCCTGGCTAACTCACCCAATGCCACAGGCATCTAGAGCCAGAGCAAGCTTTCCAGCTCTG
CCGTTGAAGCTCACCCACACCAACACCAGCAGAGCCAACTCTATTCTGCTGCTCAGGCAAGGGACAGGACTCACTTTCTTTAGTGTTATAGCTAGTG
AGGAGCAGGGCCAGCTCTCCTGTTCTCATGGCCTGGTGGCCAGCTTCCCCTTCTACCTTAGGTGGAGAGGGGTAAGGTGGAAGAGGAGGGCATCTCC
TTCTTGCTCATGCCACCCCACAGTAGACAAGTTGTGGGGCCAGCTCTCCCATGCTCATACCCACAGGGCTGGCTCACTTGCACCTCTGCTGCCAGGG
TTGACTGTACCGTGCTGCCCTGGTGAGGTGCAGGGCCCACTTTTCCAAGTCCTACAGCCAGTAATTGTCAGGGAACAATGGCCATTCTAAGAAACAC
ACATCACTGGCAAAGTCAACCACTGTGACATCAACAGGAThCAACCTAGAGTCGTTAGATAGAGGACCTCAGTTAAGGAAACGCCTCCATGATAC
TGGCTTGAAAGCAAGTCTGTAGTGCATTTTCTTAATTAGTGATTGAAGTGGGGAGGGAGTTAAGGTCCTGGGGTCCTGGGTGCTATGGAAATCAGGC
TAATCAAGCCATGGAGAGAACAAGGCAGTAAGCAGCACTCCTCCACAGCCTCTGCTTCAGTTCOTGTCTCCAGGTTCCTGCCTTGAGTTCCTGCCCT
GACTTCCCTGGAAAATGAACTACAAGCTATAAGCTGATATAAAACCTGTCTTTCCCAAGTTGTTTTATCTGTCATGTTTTATCACAGCAATAGAAAC
CCTAACTAAGACTTCATAAACTAAGAGTCAAGGATAGAAATCAATCTACTAAGCAAATGGAAGCTAAAACAAAACTGTTAGAACCATCTTCATACC
TGATAAAGTTGAGTTGAAACCTTCAAACCAAAAATAGCCAGAAGATAAATCAGATCACTGTAACGTTACTGAAAGGACAATTCTTCAGGAGCAAA
TAATAGAGACATCTACCACAATACTCTTAGATCTGATAAACATTTTTAGCAAAATAACTGTATACAAAAGCAACATGCAAAAACCAAAAAACAAAAAC
GCCATAGCTTTTTAAACATATAAAAACAAGAGATGACCTAAGAAAGAAGAAAATAGGATAACTACACAAAAATACTAGGGCTCATGTTCATTAAGAA
ACCTTAAAATGCACAAAAAACCGATCCAGGTACAACAATAGTGGAAGATGTCAATAATTCCCACTCAAATCTTTTTTGTTTGGTTTGGTTTGGTTTG
GTTTTGCCAGATAGGGTTTCACCATGAAGTCTTGCCGGACTATAACTTGCTATTCGATCAGACTGGCCTCAAACTCACAAAGGTCCACAGGTTAGGG
TTAGGGTTAAGTTCTCCTGCCTCTGCCTCTGGATGCTGAGATTAAAGACTGTGACACCATGTCTAGTCTCATCCTTAGATAGGTCTCTGGGATGGTC
AGCTTTAATTGCCAACCTAATAAAGTCTAGGGTCAACATGACCTGAGAGGAGAGTCCAGATGAGTGACTGTCAACATGGAGTGTCAACAACTTTGGG
CAAGCCTGTAGAGAATATCTTATGTAAGTTAATTGATGTAGGAAGACCCAGCCCACTGTAAGTGGTACCATCCCCTTGGCAGGGTTCCTGATCTGTT
TAAGAGTGGGAAAATGAGCTGAGAACCAGCAGGTATGTGTGCATTCATTTTTCTCTCCCCTCAACTGTAGTTTTAATTTGACTTGCTGTTTGAGTTG
TTGTCTTGGCTCTCCCAAAATGATGGACTGCAACCTAGAATTTTAAGCTGAAATACAAACCCTTTTCTCTCTAAGTGGTTGTGTGTGAGAGAGGGAG
AGAGACAGAGACGGAGGCACACACACACTCAGAGAGGGGGGAGAGAGGGAGAGGGGGGGAGGGAGAGAGGGAGAAAGAGGGGTATTTTTTAAAATTA
CAGGAACAAAAATGAAACTAGGAGTTTCCAAACTAAAAGTAAGCAAAAAACTTCACAATTAAAACATGTCAGGTATCAACTGGACTTAAGAGGTAT
CAAACAGACTGGTCCATCTAAAAGATACAAAGTATACATTATTCTCCTTTAGCCATAGAATCTTTTTTAAAACTAACCTGAATCTGTTGCCTACCTG
TGGATCCTGTTCTCCTAACTGAGCTGCCTTGTCTTGCCTCAGTGGGAGAGGATGTGCAGTAACTTGATGTGCCAGGGTGGGGTGATATCCAGAGCGG
CTCTCCCTATTCAGAGGAGGAATAGGGAAGGGGCTATGTGAGGAGGGGCCTGGGAGGAAGGGAGGCTGTGGTCAGAATGTAAAGTGAATGAATAAAT
TAATTAATGAAAAATAAAACAAAACAAGACAACGCACATTATAAACCACAAAACAATCCTTATCCATCAAAGGAACATTTGAAAACGCTTGCACTGC
ATTATACCATAGTAGAATAAAACTAAAATTAAAAAGTAACAGCAACAAAACCAACACACATTCTAGGAAAGTGAGCAATGAGTCATTGAAGAAGCAA
GGGAAGAAAAATTTCAATCCCTAGAGTCAGATGAAAAGAACTGCTCAACTTACTAGAACTTCTGTGGCACATCTAAGGCATTCTGCAAGGAAGGTG
ATGGTCATATGTGTTTATATTAGCAAAAAGTATCAGGTAGACCTCAAATACATAATTAAATAATGGGGGGGGGGAGAGGGAGAGAGAGAGAGAGACC
AAATTTGTATGCAGAAGATTGCAAGAAAAATAAAGAGAAGGTCAGAGATCAATTAAATGGAGACTAAGAGAGCAATGCATACAGCCAGTCAGAGAGT
TGGTATTTGAAGAGATAAACAAGTTTGATAATCCTTGCAGAGCAAAAAGGAGAAAACTCTCAGATGAAGAAGTAAGAACAAAGCATGGGATCACTA
GGGAATACTTTGAAAATTAATTTCAAAACCTCAGAAGTCCAGAGGAAATGGATACATTTCTAAACACATATGGTCTGCTAGAGTTAAATCAGGAAAA
TACAAGCAGCATAACTGGACTCAAAATAACCAAAATAACAACCAGTTGTTGAAGCAGTAACTAAAAATCTCCACACAAAGAAAAGTCTCAAACTAGA
TAGATTCACTGCTTAATTCTACCAACCTCTTAATGAAGATTTAACAGAATACTCTTAAATAGATTCACCAAATAAAAAGAGAAGGAACAAGACCAAA
GTCATTACACAAAGCCAATAAAATCCTGATTCCAGAGCCAACGAAGGATACAACAACAAAAACTGAAAACTGTAGACCAACTTCCTTTAAGAATTCA
CAAGCTCTTTAAGCGCTCACCTTTCTTACTCTCTAGCTCTCCTTCTCTCTCTCCCTTCTCCCCCTCTCTCCACATGGCCTCTCTCTACCTTCTCTCC
TTCCCCCCGCCTTTCTACAATAAAGCTCTAAAACAAAACAAAAAAAAAGCATTCACAACAAAATACTTGCAAACCAAATTCAGAATCCAATAGACAA
TGCATCTTAATCGAGTTACTTTCATCTCAGGAAAGCATGATTAGTCTAACATATGCAAATCAATACATGTATAAGGACAGAAATCAGAAGATTATCT
CAAGAGATGCAGAAAAGGCATTTGAAAATTCTCAATATCACTAGACATCCCGAAGAAACTAGGAATAGAAGAAACATTATAAAGACCTTGTAAAAGC
TTACAGCTGACTAAATGGGGAAAATCCAAGAGGATTTCTTCTAAACTCTAGAACAAGACAAGGTCCTCACTGTGTCTATTATTATTCAATATACTGC
TTAAAGTCTTAGCTAGAGCGACAAAGGAGGTGGAAAAATAATGGGAATATAAATAAGGAAGAAGTCGAGGTATCCTTATTTGCATATAGAGCTATCC
CTATTTGGAGACAGCATGCATACATTTAAACTCTACTCAAGACTCCACCAAAAACCTCTTAGATATGATTAAATAGTTTCTGCAAAGTAACAGGATA
CAAAACCATACAAAATCAGTAGCTTTTCTATGTAGAAATATGAAACCACCTAAGAAAGATAAGTTAAAAAAAAAAACTATTCACAAGAGCTTCAAAA
AGAGTTAGATGTGAACCTAATAAAGAATATGGATGCCTCTAAAGTAAGGCCTTTAAGACACTGAAGAAAGAAATTAAAATTGATATTAGAAGATGCA
AATGTCTTCCATGCTTATGGAATGGCATAAGCTAATATTGCAAAACTAACTGGACCACTGAAAGGGAATATAGTTTAATTCAGTGTCCAACAAAAGT
CTAATGTTTTTGACAGAAATTCTAAAGTTCGTATTGAATCATGAAAGTCATTAAAAAGCTAAAGCAGTCCTGAGAAGAAAACAATGGTGAAGGGATC
GAACTATCTGACTTCAAATTGTACCACAGCACCACAGTGACAAAGACGTTGGTACTGAACAGATTAAACCAACAGACTGGATTAGAGTAACCAGAAA
TTAGAGCCCACACAACTGTAGCCAAATAGTTTTTGATAAAGGGGTCAAAAACATACATATATACGTTAGAAAAAAACATAGCTTCTTTAAATAATAC
TAGGAGACCCAGATACCCACATGTCAAAGAATGGAATCGGATTAGTGTGTATCGTACTGCACAAAAATAAATTTGTAATTGATCAAAATCCTTAATA
TAAGGTCTGAAACACTGAAACTTCTAGAGGTAATTCACTCAAGACAGAAGNNNNNNNNNNNNNNNNNNNNNNNNNNNNNNNNNNNNNNNNNNNNNNN
NNNWNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNWNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNTTAAAAC
TGTCCCCAGACTCAGAAAACAATCACCAACAAAGCCTGGCAAGGACAGGGGTGGAGCAGGGGCTGACTCCTTGTCAGTGTTAGGGCAAGTGTGAACC
CATATGGCCTCTATGGAGATCAGTATGGAGGTTTCTCAAAAACACTAAAAATAGAACTACCAATGACCCAGTCTTTTAAATCCTGGAATAGACCAAA
GGATCCTAAGTGCAATCAAGAGATGTTTGCCCATCCATGTTTATTGCCGACCTATTCACAAAGGGATACCCTGAGTGAGACAAAGAGGAGACTCAAA
GGAATAAAGAAGAGGTAACTACATGGTAGACATAGAAAAGATTTAATGGGTTGAACAAGTTATGAGTGGTCAGGGAATGGGCCAAAGTTCATGGACA
AGGCATTAATAATAAACAATTAGTCTCAGAGGTGTCTTTCTGAGAGCTGGGACTAGOAAGGAAAACCAGATTTTTTTTTTTTAACAAGGAGAATCTC
AGAGCTCCCTTCTCTACAGGTCTAATCTCAGAGATTTGTCCTAGGGATGGACCACAGCGGTGGCTGCCTACATCTGCCTAGGTCTCATTGGCCCTCC
ATCCTTATTCCCTGCGTTTTATGTAACACTGTGACCAAGGAGATGTCCACACACTCATTAAAGACTTCACATAGAAACAAGGATCAGAGTTGAGTGG
CCCAACTTTGOAGACCTGGGGAAGGTTTTAGAGCTCAAGTCTTTAGTCCAAGAGAATGGATGGATAGACGCATGCTGACTAAGGAGGAGATAGATGG
CTACCCTTTTGTTCTTACATTGTTCCTGAGATTCGCAGAATGGCATCTCACCCCAGAGGTAACTGAGTCCAACAGGCAGGGAGCCCCCATCATTTGA
TATTTTCCAGATTTGCTGTATGCCACCAAGAGAGCACACATAGTGAGAAAGGACCGTGTAGCCCAGGGCACCGGCTWTGGAAAGTGCGTGATGGGAA
TGGATOGAAAACATTGCTGTGGGTGAACTTTGTTGTTCCGGGTCTTTATTCTGGTGATGCTGTTGTCTTTGTTCAGACATCTTAAACTCAGTTGTTA
TGTCAGTATTAGTAATTCCAATTTCTTCTTTAATTTCCATGCAAGATAGGGCTTGCCTGTAAAGTGOACTGACAGAGGCAGCGTTCTATATTTAGTG
AGGCAGATGCTATCTGCAAATTACAGGGAAATGATTGCATCAAGGTCCCTGGTTGGTGAAATAAACOACAATAAAGAAATGAGGAGOCAGAGAGGGG
CAATGTTCTAGAAGTCACTAAGAACCAAAGTTGCTTGACTCTGACTGGTAATGATTTCTTGGTCCCCAAATGCCCTCCCATGCCCAAGCAGCATGGC
CTTCATGCCCTTGGGGTTAGAGGAAGAATAATCACATTGAACTAITTCCTCTACACCCTGAGAAAATGATTATCATCTTCACCGGACAGTTAAGTGC
CTAAACCTCAAACCACCAGGGCAACTGTGTGATTTTAGGAAGGACGAGAAACAGGGCATCTATTTTGTGACTTACCTAAACCCAGGAGCTATCAGAG
GGCTTCCTGACACTTTTAAAAGAAAAGAAAGCGTAACTACAAGGCAAGCATAGGGCCTTCCTGGGCCATGATGGAGCCGAGGGTTCAAGTTTGTAGT
GACCGTGCCATTTTCTTCCCATCCTGAATACAGAATTCACTCTGGGATTTTCATCATAGACAAACACAATTCAGGTCACCAGAGTAAACTGCTTCTG
TAATGGTAAATTTCCCAGCAGGTCTTAAAATAAATAGATAAATAAATAAATAAACAAACAAACPLAACAAATACACACATACATACATAACATACACA
CATACATACACATATATTTTATAATAGTCTGTGCAACCTAATGTTATTTTTCAGCTCACATTTATTTAATTATTAGGAATCCAGGTTACCTTAATTT
TTTTGTGCTTGCTACACACTTTCATGCATACCAGGT
MOUSE SEQUENCE-mRNA
GGGACCCTTTGCTGCCCTCAGCTAAGAGTGCGGGTGAGCGCACAAGCCCAGGAGGAGGTCCGCACGCGTCATGAACTTGGAGACGGGCAGCCGGGGC
TCAGAGTTCGGCATGAGCGCAGTGAGCTGCGGCAATGGGAAACTCCGACAGTGGTTGATCGACCAGATCGACAGCGGCAAGTACCCCGGGCTGGTGT
GGGAGAACGAGGAGAAGAGCGTCTTCCGCATCCCGTGGAAACACGCGGGCAAGCAGGACTACAATCGTGAGGAGGACGCTGCCCTCTTCAAGGCTTG
GGCATTGTTTAAAGGCAAGTTCCGAGAAGGGATCGACAAGCCAGATCCTCCTACTTGGAAGACAAGATTACGATGTGCTCTGAACAAGAGCAATGAC
TTTGAGGAATTGGTCGAGAGGAGCCAGCTGGATATCTCTGACCCATACAAGGTGTACAGGATTGTTCCAGAGGGAGCCAAAAAAGGAGCAAAGCAGC
TCACTTTGOATGACACACAGATGGCCATOGGCCACCCCTACCCCATGACAGCACCTTATGGCTCTCTGCCAQCCCAGCAGGTTCATAACTACATGAT
GCCACCCCATGACAGGAGOTGGAGGGATTATGCCCCTGACCAGTCACACCCAGAAATCCCATATCAATGTCCTGTGACGTTTGGCCCACGAGGCCAC
CACTGGCAAGGCCCATCTTGTGAAAATGGTTGCCAGGTGACAGGAACCTTTTATGCTTGTGCCCCACCTGAGTCCCAGGCTCCTGGAATCCCCATTG
AGCCAAGCATAAGGTCTGCTGAAGCCTTGGCGCTCTCAGACTGCCGGCTGCATATCTGCCTGTATTACCGGGACATCCTCGTGAAAGAGCTGACCAC
GACGAGCCCTGAAGGCTGCCGGATCTCCCACGGACACACCTATGATGTTAGCAACCTGGACCAGGTCCTGTTTCCCTACCCGGACGACAATGGACAG
AGGAAGAACATTGAGAAGTTGCTGAGCCACCTGGAGAGGGGACTGGTCCTCTGGATGGCTCCAGATGGGCTTTATGCCAAAAGACTCTGCCAGAGTA
GGATCTACTGGGATGGGCCCCTGGCACTGTGCAGCGATCGGCCCAACAAGCTAGAAAGAGACCAGACTTGCAAGCTCTTTGACACACAGCAGTTTCT
ATCAGAGCTGCAAGTGTTTGCTCACCATGGCCGOCCAGCACCGAGATTCCAGGTGACTCTGTGCTUEGGTGAGGAGTTTCCAGACCCTCAGAGACAG
AGGAAGCTCATCACAGCTCATGTGGAACCTCTGCTAGCCAGACAACTGTATTACTTTGCTCAACAAAACACTGGACATTTCCTGAGSGGCTACGAGT
TACCTGAACACGTTACCACTCCAGATTACCACCGCTCCCTCCGTCATTCTTCCATCCAAGAGTGAGAAGAAATACTCTGACAGGGCAGCCGGCTGCT
GCCCTTTCTCTTTGGAAGAGCTAAGAAGTGAGTGGGTTTCCACTTGAAGACAACAACAGGGCTTTGTGAGGAAAAACAGCTGTATCTGCTCAACAGA
GGAGCTTCCCCCAGAAGAGTGCCTGTCATCCAGGTCTTGACAAGTGCCAGGACTTGGGTGACTGTGCCCTGGCTTATAACTGTGAAACTTGATCAGT
GTTGTGTTTACATGTACTTGAATGCTGGCTTTAGCCTGGTATAGATGGACTTTTGCTTGAAGACTGAAAACCTGTGCCAGCATGAATCCCTGACAAG
AGAAGACATACGTATTATTGGTCCATTTCTCAGGGAAGTAAAGTCTAGACTAGAGACTACGCTGTCCACTCACAGAGAAACGCATTCCTGGATAGAG
GAGCTGTAGGTTGCCCAGATCATGTCCACTGAGTGAAGGGACTTGGTTCTTAGGTCCAGTCTAGGCTATCATGTCCTGATCCTGCACTCTCTGAG
TAGACCCTACATCCTGTTTGCTCCCTCTTCTTGCCCTTCCCTAAAAGCCAGCTGGTGGACTCTTGTTGCCACATACTTGAGCCAAAAAACCATCAGG
GACATATAAGATGAGACATGTTTGCATCTCCTCTGATCTAACAACGGCTACAAAGTTTTTTGTTGTTGTTGTCTCCCCTTTCTGTTTTGCCTCTCAT
TCTTTAGAATTGTTGCTTTTTTGTAAAGAAGGAAGAGAGAGAGACCGAATATACTAGAGATAGATTTTTAAAATTCTGTGCCATTTCCCCTTCTAT
TTGTTTTGTTGGCCATCAGTGGTCCCCATGGCAACACTGGAAGGGCGGAGCCTTGAGCAGGTTGGGAACTGTCCCGGACACCAGTGGTTCAGTGTA
CAGCTGTAGACTCAGTGAGCTGTTTCTGCTTATTTTGTATATTAGATGCTTCCTTGTGCCAATAATAGTTTGACAGAGTCTCAAAGCTACCGGGCTT
CTTTTCCAACAACACATTCTTTCACAGAATCCCAAAGAGTCTCACTTGCCTTCACAACCGTCTGATCCTTTATTGTTACTGTTTTGTTTAGGTGGCT
TTGCCATGCTCTGGGATTTTTCTCCTACCACAACATCATTCCTGACTCTTTCGGGCTCAGAAAGAGTTAAGCGCTCTTGCACACTTGGCACTTACTT
CTTTTTATTTTTTTTTTACATGCCACTCTTAATATTTCTTCTTAAGTCTTTCAGCAGCATACATTCAATAAGCAATGGACCATTGGGTGTCTCAT
TTCATTGTTTTATTTTTAAAAAGGAACCTTTGACAAATAATACACATTACAGAAAATGGAAGCATTTTTCATAGAGAAGGTAAGAACTGAAATGCAT
TTTATGTTCGAATCCTCCTTGCTAGTATTAGCCCCAAGAGTTCAGTCTGTCCTTTTAGCATGTTTTAGTTTTCAATACATATATATGAAGTTGCCCT
TTCTTAGAGAGAGAGAGAGAGATATGTGATGACACTCTTATGTACCCAACTATACAGATTTACCTCATCTTTTGAGTGGCATGTGGACTTCCACTGC
TATGTGCCTAGGTGCAGTTTAGACATCTTCTTAGCTACCCATCCCAGCAGCCCACTGAGTCTCGTGAACTAAAGTCACTTATACATCTTATAGATGC
AAAACATTCTGGGGTATATTATCCCAAGGGAAGATGAAGGCAAAATCGGGAAGCACTCTTCCTGAGGCCCCAAGCCACAGCTGTCCTCCTGGAGTAT
CTTAACCATATCTGGCACTTCACCAAGGTCCCTTGCCTGGTCCTGTATGACCATGACTCTTCAGTCCATAAGCAGATGCTTTGTCTCAATTGCCATG
TGAAGTCTTTTGGTTTTTGGTTTTTTTCCTTAATGTTAAGCTGTTTTAACAGTGAAGCTGAATTTTCTGGAAAATGCTGTATGGTTCGAGCCCCCAG
TTCATGTTTCACTTTACAGCCATGCATGCTGATGCCTTCCATCTTGACAAATGGAATGATGGGTAAACACTCCTGACACAAAGAGGAACACCTTCCT
CGTCCCTTGCTGAAACAAAGTGCAATGCACACAGTAGAATTTAGTTTGCTTGCACTGGAAGATGCCGTTGAAGAGGTAGGCTGAGGAACACTGTATC
CTAGGGAGAAAATATGCAAGTCTCACGGGTGCTTTCTGTTGGCTTGTTTCTGGTCCTTGAGCAGGCTTAAGTATCTAGTTTCAAACTAGAAGCCCCA
AAGCCCTCAGTCGTTGTCCTGGACAGAGTTGGTAGACCCCACCTGTAGAAATCCAGATGCACTCTGGGATTGGAGCACAGAGAGGCTTGCTCTATCG
TGAGAATGCCCAGCCTGCAGAGGCCCAGGTGCGGTGCCTGAGTGGCTGTATGCCAGACACAGGAACGCCAGCAGGCTGAGTGTGGTGACTGATATGT
GAGGTGCCCTTGCCCTGCAGACAGGCTGAATGTGAGCCCCAGCAGCAGAGGATTCTGAGAGTCAGGGTGGTGTCATTTCCTACACAGGAAGAGCCTT
CAATATTACTTTTTAAACGAACTTTCCCATTGCTGATATTTTGATAAAAACCCACACAAGGTTTAATTACATGCCCCCTCATCCCTCTCCACACAGT
CTTGCCCATCCCACTGGTGAATTGTTGTTGTTCTTGTTGTTGTTTTTTGTTATTGGTGAAAATTGTGAAATTGTTAAGGTCTCTGTTTTTGTTATTT
ATCTCCCTAAATCACATGGCTGCTGCATACATTGCAATGTTGCCCTACCACATGTATGTTTTTGCAAGTTGCTTCTTCACTCATTGAAGAAGCCTGC
ACCGTAAGTGAACTACTTAGTCCTTGCTAATTGCTGCTCACTGTCACGTGACGCAGAGAAGGCTGATTTTCCTGGACCCATTTTCAGTGCTTTG
AGGAACATGAGTCCAGAGTATATTGAAGTAGAAGACAGTGTTTGGCAAGCCAGTTCTTATTAGACACTGCTACAGGCTTCACAATCTTCAAGGTGGA
CCCACCTCATGTGGAAGGGCATCATTGCCCCTCTGCTCCCATGCGCTGTGCACCCCCTTCACAGTGAAAGACCTGCTGGGGACCAGGACTCAGAGAT
CTCTGGCTTCGAGCTCATATCTTGGCTTCCTGTCATGTCTAAGCCCGACAGGAAAGTGTGGCACCATCCTGCCTGCGGCGTTGGATGGATAGGCCAT
GGATTTTGCTGTAAACTTGCTTCACAA
MOUSE SEQUENCE-CODING
ATGAACTTGGAGACGGGCAGCCGGGGCTCAGAGTTCGGCATGAGCGCAGTGAGCTGCGGCAATGGGAAACTCCGACAGTGGTTGATCGACCAGATCG
ACAGCGGCAAGTACCCCGGGCTGGTGTGGGAGAACGAGGAGAAGAGCGTCTTCCGCATCCCGTGGAAACACOCGGOCAAGCAGGACTACAATCGTGA
GGAGGACGCTGCCCTCTCAAGGCTTGGGCATTGTTTAAAGGCAAGTTCCGAGAAGGGATCGACAAGCCAGATCCTCCTACTTGGAAGACAAGATTA
CGATGTGCTCTGAACAAGAGCAATGACTTTGAGGAATTGGTCGAGAGGAGCCAGCTGGATATCTCTGACCCATACAAGGTGTACAGGATTGTTCCAG
AGGGAGCCAAAAAAGGAGCAAAGCAGCTCACTTTGGATGACACACAGATGGCCATGGGCCACCCCTACCCCATGACAGCACCTTATGGCTCTCTGCC
AGCCCAGCAGGTTCATAACTACATGATGCCACCCCATGACAGGAGCTGGAGGGATTATGCCCCTGACCAGTCACACCCAGAAATCCCATATCAATGT
CCTGTGACGTTTGGCCCACGAGGCCACCACTGGCAAGGCCCATCTTGTGAAAATGGTTGCCAGGTGACAGGAACCTTTTATGCTTGTGCCCCACCTG
AGTCCCAGCTCCTGGAATCCCCATTGAGCCAAGCATAAGGTCTGCTGAAGCCTTGGCGCTCTCAGACTGCCGGCTGCATATCTGCCTGTATTACCG
GGACATCCTCGTGAAAGAGCTGACCACGACGAGCCCTGAAGGCTGCCGGATCTCCCACGGACACACCTATGATGTTAGCAACCTGGACCAGGTCCTG
TTTCCCTACCCGGACGACAATGGACAGAGGAAGAACATTGAGAAGTTGCTGAGCCACCTGGAGAGGGGACTGGTCCTCTGGATGGCTCCAGATGGGC
TTTATGCCAAAAGACTCTGCCAGAGTAGGATCTACTGGGATGGGCCCCTGGCACTGTGCAGCGATCGGCCCAACAAGCTAGAAAGAGACCAGACTTG
CAAGCTCTTTGACACACAGCAGTTTCTATCAGAGCTGCAAGTGTTTGCTCACCATGGCCGGCCAGCACCGAGATTCCAGGTGACTCTGTGCTTTGGT
GAGGAGTTTCCAGACCCTCAGAGACAGAGGAAGCTCATCACAGCTCATGTGGAACOTCTGCTAGCCAGACAACTGTATTACTTTGCTCAACAAAACA
CTGGACATTTCCTGAGGGGCTACGAGTTACCTGAACACGTTACCACTCCAGATTACCACCGCTCCCTCCGTCATTCTTCCATCCAAGAGTGA
HUMAN SEQUENCE-GENOMIC
TCCTGAGATCTTATCTTTGATATAAGTTAGCTCCATTTACACTTAGCAGATAATACCAAATTAAAACAGAGACTGAATTCAGTGAGCAGAGAAAACA
GCCTAGCCCATGGATTTGTTCTCAATGCACTGCCTCCTGGCTTTGGTGATTTTGCAAAGAACTGAAAGAAAAAGCACAACATTCAGAATAGAAAGGG
ATTTCTGAAGGGAGTTTATAACTGAAATTTCAGTTTGAACTGGATGAGGGACGCTGTTTAGTTCAGTGACCACTGAGGACCTCCGGCAGGCTTCG
GGGTGTACAAAGACAGCCAGGATTCAGCCCGCCTCAGTGGAGGGCCCCACAGCACAAAGGGACCAAGAAGGGATAGCAAGAGGACAGCCAGAGTGCC
CACAAGCTCGGCAGGAAGGAGGCTCACATCGCCGCGGAAGTCTCGTGATGGTTCTGGTAGAATCGTCCTCTTGAAACCTTCTGCTGCCATTATTGGG
TTCTGTCCAATATGCOTTACGCAAAACACTGACAATAGCATACACCTTTCCAGAATGCCGGCACGCGCTGTGCAATGCCCAAACTGTCCCTGTGAGT
TGAATGCCCCTGGCAGGATGCGTGTCCACTCCCGCAGGGCCGTGGGTGCCTCCCTTGTCTCACTCCTCTGCTTCAACCCTTCCCAGAACTCTGAAGG
TTTGAGATGAAATGACTGAGCCACACCTAGTGCAGGAATGAGCACCAGGGCCTCTCTGCAACGGCTGCTTAAGGGCCACAGGATAAGCATAGTGTGG
CACCCCCAGTTATTCCCTGTTTCGCTCATTGGACCACCCTGATAGGCTTCAGGGAGGCAGGAACCTCCTCTAAGGCAGCAGGAGAGGGGTGGATGGG
GCAGCCAACAGGTGCATCTCATGCCTCCGTGTTCTCCATAGGGAGGGAGGTGGGCTAAGAGGCGAGTGGGGCAGAGGCCTGGGCCAGCGTGAAGTGT
CAGGAGTAAGGGGGGAGAAGACTGGAGGGGGCAAATCCAGGAGGCAGCTGAGGCTGCACTGTGCAAATACAAGCCCGGTTACGTCTGTGTAGCTTAG
CTTCCCCAGCCCACCTGAGCCAGAGCTGGGTCTGCCCGTGCTCCCGCTTCTATTGACATCCAGCAACTCCAAATGACCTCTCCCATATCAATGGGCT
CATCTCCGAAGCCGAATCTTATAGCCTGCTTCTCCTCCCCACCAGGCTATAGTGGGAAATCATTGAGGAAACTATGGTTGAATGCATCGCACGGCCT
CTCTGGTGTCTGAGAGAAGCTTCATAGCCAGCAGGCTGCCCCTGGTGGGCAGGTGCCGCATGGAGGTACCACGCCTCCCGTGCAGCGAGTAAGAAGG
GCAAGGAGAGCTTGTCAGGGGAGTGGGCCAGGCTATCACATCAGATGGGCCAGGCCTCGCATCGCTCTCTGCATCCTAAGCAACGTCTGGACTTGAG
TGCGTGGGTTTGTGGTACTGCAATGCOCAGTACCTTGGGGAGCCCCCAGGCAAGCCAAGCCAAGTTCTGGGAGGCCACAGCCCTTCACTCAAGTTCA
AAGGTTCAAGCAGGGATTCTGTGTCACACTTGCGAAGGTGCCCACCCCAACAGAAGCTGTGGCTCTGCAGGACACGTGCGGGGTAGACCTCTAAGG
CCTTGTCCCAGTGGAAATGCCCTCCCACTTGGCCTGGGTAGCTGCTCACGCTTCTGAAACGGGAAAAATCAACTGCATTCCTGAATGGGAGCCTCAG
AGCATTAGAAAGATAATGTTCTGATTTTTTAAAGCTAAACGTATATGGAAGGAAGCTGAAAGAGGACTTTTGATGAGTCACCTAATGTTGATGTTG
AAATAACCTAATGACCCTCTGGCTAAAATCTGTTCTCATTTGGGAAGCACCTGAACCCTCATTCTCCCGCCACTGCTCTGACCTCTGGAAAGCAAG
GAGCAAAGGCACACTCAGCAGCTCCCGCTCCATGCTCTCCCGAGTCCTCCCAAGCAGCCAGAGGACGACTCCACACCCAGAACCCAGAGTGGCC
TGGGAACCAGGCCTCAGGTTCCTGGCCCATTTGTCATCGCCCAGGGTCCTCCTGCCAGGAATCCCCCCACATCCTGCCCTGCCTAGAGAAGCCCAGC
TCTTCCATCAAAGTTCTGCTCAAATGTCACTGCATGTTACGTCTTCCTCACACACTCCCACTAGCCTTACTGCATCATGCTTGGTTTGCTGAGCTAT
GTGTGGGGTCTTTCCTTGCCAACCCTTCTTTCCCTGAGAGGTGCAATGACCTCATATTGAGGCTGTCCACTCCCCCACTCCCGACCCCATCCACCAA
GCCTGGGAGGCACTGGCTTAATGCATCATGGTTAAAAGAAATGGATGGTTGAATTGCACAGAGAAGACACTGTGCCCCAGGAGATGTGCCTTGCACA
AGATCCCTGCAGCACATGCTGGGAGGCTCTGCCCGGGTAGCACTTAGGAAAGAAGGATGTATGCCCTGGGCTGCTGGGAGCACTGTGGGAACTCTGG
GATCCCCCTCCTCCTAAGGGCTGTGCTACCCTGATTTGGAAACAATACAGAGGCTTCTCTCCTGCAAGAAAACATGCCCCCTGCCCACCCAGGATCC
ACCTGGCTGGGGAATCTGCCTAGCTGGGGACCAGGTGGGCATGCTAAGGAGGAATGGGGCTGTGTCTGAAAGAAAGAGATGCCAGCAGGATCCAGGA
GAGCAGTCACGGTCCTGGGGTCTGAGAGTGCTGGGCCAAGGAGGTGTGGGGAGCATAGATTCATATAAGAGAGAGTGGTTCAGTCTGTGGCCATGCT
CCTGGGACACAGCACTGAGCACCCAGCAAGGCCCCAAGAGATGCTGAAACACACAGCTAGGATGGCTCCCAGGAACGTGAGACAGCATTAACCACAG
GAAGCCAGGCAGAAGTGCTGCAGCCAGGGCGTGCTGTGGGAGAAGGGCTGGGAGOATGCAGCTCGCTCGCACACTGCGTCAGAAACCTTCACATAGT
GCTTGTCCTCAGTACGAGCCAAGGTGGGGGTGAGGAACGGCCCACTTACCAAAACTCTCAGAGACCCCTTGGGGAATTTGTGCTTCTTGTTTCTGCA
AGTTTCCAGGAGACACAGAAAGGGTGCCACTAGACTCTAAGCTGTGGGAGGCCTCTGGGAGGCTCCTTGGGTGGAGCTTAGCAGCAAGAAGGCATGT
TGACCTTGGTCATCAGGAGGTGTGGAGCTGCTGCTATACAATGGGGCAGGACCCCGGCAACCCATCCGGGCACCTCTTGGCTCTCCCTGTGTGTATT
GATGGAAAATGGTATGTGGAGGTGCAGCACCCCTGGACCAGGAAGGGCACCGTGACCAGTTCAAAGCCCTCAAGGATGAAGGCTTGGGTCTGGCCAC
TGAGCTGTAGTTGAAAGGTGGGAGCATCTAGAAAGGACAGAGCAGGAGGCGCTCACGAGTCTTCACTGGCGTTCCAGCTGCAGGGGCTGAGGCTGGA
ATCTGTTCCACTAACTTTCCTCTTCTAAGTTTTCTCCAGGATAAAGTTGCTATTTGAATTTCAGATAAACAACAAGTAGTTTCTTTAGTGTAAGTAC
GTCCCAAATATTGCATGGAATACACCTATACTAACAAGTTATTCAATGTTTATCAGAAATGCCAATTTAACTGGGAGTTTTGCATTTTTATTTGC
TAAAACTGGCAACCCTCCTCCAGGGATGGAGGCCCACTGGAACCAGGAGGAGCTGCCCTCAGCAGCTATGAGAGGAAATGGATCACAGCAGAACGAG
GTGTAGACTTGGGTGGATGTTGCTGTGCACTGCTTGGATTCCCACTTGGGAGTGAGAGGTTCATTCCCTCGGGTACTGAGACTGCTCCTGGGAGGTG
GTTCTCAGCAGGCAGCCTTCTGTGGCGATGGCTCCAGCTGGAGAAAACGAACTTCCCAGGTCACGTCTCCTCTAGAGGCAGCTGGCACCCAATGCTA
GCTCAGTCAGGGCTGCGGGGGCCOTATCCCTTCACTCCACGCTGGAACAGGCTCGTCCTAGCTCCAGAGCTTTCTGCAGCATTGACTGGAGTCCATT
TGGCTTGGCGTTGCATCACGGGCACCTCTCGGCCCAGTTCTGCTCCCTTCCCCTTGACTCCAACCAAGCATGCTCCCTAATCATACATGCTGACCTC
CATCTCAGAGTCTGCCACCGTCCCTGTTAAAGGCTGAAGGAGGACTAGGATCACTGTCAACAAAAGCATGACCCAAGAGGCAAGACACAGTCATGTT
TATAGCATATTACTATGAGCCACTCACTACTTCTAGATGTCACGGTTCAAAGGTGATTGAAAGAAACTCTGTGTTTCCCAATGCCTGCTGGATAAAT
AACTCAGCATGGATGGTGAGACAGCATCTCTAACTTCGCCTGTCTCCCCGGTAATCGTGTTCCTCCTCCTCACTTCTTTATCAGGGTTCTTGGTAAT
AGTAGTCCCCCCTTATTCTCAGGGGATAGATATGTTCCAAAACCCCTAGCAGATGCCTGAAATAGAAGACAGTACTGAGCCCCTTATAGACTATGTT
TTTTTCTGTACGTACATAGCTAGGATAAAGTGTAACTTATAAATTAGGCACAGTGAGAGATTAACAACACTTATAATAACACCAAGCAATGATGACC
ATACGCTGTAATGAAAGCTGTGGATGTTGTTGCTCTCTCCAACATCTCACTGCATGGCACTTGCCC2CTAATGGCCTGATGGCTGCCACATGGCTGA
TGGGCCAGCAGCATATACAGTGGGGTCTGCTGGACAAAGCAGTGGTTCACATCCCAGAAGGGATGAAGCGGGACCCTGCGCGATGTCACCACCCTGC
AAAGAATGGCATGCAACTTCAAACACAAAGTGTTGATTTCTGGAATTTTCCATTTCTTATTTTCAGACTGAGGTTGCCCTCAGGTAACTGAAACTGT
GGAAAGGGAAACGGTGGATACAGGGGACTCCTGTGTCATTCCCCATCOTGGAAACCCTCCAGCAACCCCTGACTCCCCGACCGCCCCACCCCCTGCC
GAGCACGTCTACTCAGCCCCATAACTGCTTGTCCTTCCCTCCTCTGCCACCCATGCACCTGCCCGTCTGAGAGCTCTCTACCTCACCCCGAGGCCTC
CCCGGCCTCCTGGCCATTGTTCTCTCTCGGGCGGTACCCACACTATGGCCAGATAATTCCTCCTTTACTGTAGTTCTTACCTTATTACGGGGGAATA
TGAGCCAAAGCCATGTAACTCGCTGCCGGGCAGGAACTAACAGAGCCAGCACATGCTTCTCCCGTACCCTTCTCTCCATCAGGTGGCCAGGAATGTT
CCGGATCCCCGTGGCTTTGCCATGGATATCTCATTTGAACAAGAAGTAACCCCCCCCCCAGAATCTCAGGGGGTCTCGTTACCACAGCATAACCTAA
CCTTTCTAGAAAGATACACTCCTCTATCCAGCATGCCTAGAGTCTTATGTGTGACAAGTGACGGTATGCTGTTTCACAAAATTTCGCTGAAAGAGCC
GTTGATGAACTTTAGAATAACCCAACATTAGGCGCTGTTACAAATGGGTGGAATTAGACAAAGCCGTTTGCCTTTCACCTCTTGCTTCAGTTTTCAG
AAAATTGCCCAAAGGTATTGCAAACCATTTGCGTTTTCATTGGCTTGGTTAAGGAGTTGGCAGTGTCAACTGGCCCTGTGTAAAGCCACGTCACTGT
TTCTGTAGAGAGCATGACACGTCAGAGAGGTGGGAAGTGTTGCCAGGACTCTTCTTAGAATGCTGGCAAGAAGAGCAGGCGAAAAGGGAAGAGCTGT
TTATAAACAGGGAAAGGAGACATCACCTCGTATCCTTGGCTTCTCATTACGTCATCGATTACAATCACAAGTCTCATTTCCTACAAGCCTGAAAGCG
GGTTCTTCTCTGCATGTAGGAAAAAGCCCCGCTTACTAACTCTGCCCTCAAAGAGATCACAAGCTGGTCCTGAACGCCCTGTGCCCTGGGAATGCTT
ACTGTCGAAGCTCTGCAAATTTTCTCATTGAAGGCACTCAGGGCATTCTGCTAATTAAATTAGGAAAATGTGTACAGCTCTACTTGAAAAAATCCAG
GTTTATGCTCTGTATTCTATTACCAAAGAATTTATCACTTTTCAAATAACATTCAATTATTTTAAACTGAGTGTGCAGTGAGAGCATTTATAATATA
ATCTCATTTCCAGAGTGTTCCATCCCAACTCCTCTCTGGTATAAGTAGCTTAGAAGGTTAGATTTTTTTTTTCCAGAGGATTAAACATTCGAGAAGA
GTGGCTAAGAGGCAGCACCAGGTTTCTCTCATTTCCTCAAAGAGAATTTAGCCTTGTGAAGAAGAATAAAGTGAAGGTCAATGGTTAGATCCATGGC
TTAAAAATAAAATGCAGCTTTCCTTACTCTGAATTTACCATAACCAAAAAGGCCACGAGGCAGCCATTAATGATCCTAAGTTGCAACAGAAAGAAAA
AAACACCTACTTAGGGTTAAACGAGGATTCAGTGGTCACTTGCCGCCGGCCTCCACTTCACAGGGGCGGAGGATGAGGCCAGGGAAGACTTTCTTAC
CCACACTGACCTGACCAGAGATGAAACTCCCCCTCCTATGACAGCCTGCACTCAGAGGCTGATTCTGAAThGTCATGCCAAATGCAGCCAATCCCT
GTGTACAAGGAGAAAAAATAGTTCCTCAGAGCTGTGTCCTTGGGCCAGCCATCTGGACCTCTCACAATCTCTTTCGTTCTCCCTACACTTGGGCTGA
ATCCCGCCTWCTTGCCTGCCAGGTTTGGTTTGGGGGCCAATGAGTCAAAGTCCGTGAGATACCCCTAAGTATTGAAGTGCGTAGTAAATGATAAT
AGCCCTGATTTGTTGACATGATGATGTGCCAGGCATTGCGCAAGCATCATTTCATCATCACAACAAACACTGCTGGGAGGCAGGGACACCCCCAC
CCTCCTTGTACACCCAGAAGTAGGACTTTGGTTAAATGGCTCTCCCAAGGCCTCACAGCCTTAAAAACTCAAGCACTTATTTAATCCCCAATGATAC
TACCTTTTGGCCATGCAATGTGTCATGGCCAGTATTAGATTTGCAAACACATATAACCAAAGAGGATTAGAAGAGACATATATTCTATATAAATTA
CATGAAAATAGCACTTTCAGTATTTTCCTGATTTTTTCTGGTAATTAAAAATGATTGCCCTTTATATTTCTTTATGGATACAGTTGCATTGTTTTTA
GCCAATTGTAGCCCCCCCCTTTAAAGAGTGAATTAGTGGGAAAATAGTGTTATCCAAGATTTTAAGAATCTAGTGTGTGTGTTTGTGTCTGTACGAT
GGGTTTTTCTGAAATATTTTACTTGCATGTGAATGTATAATTTTAAAGATTGGCTTTTAAAATACTGAATTGAGGTAAATTCAGCTTTTCTTGCAGA
CTCTTTACGATAAATGTGAAATTGAAATGGAAATGCTCCATTCAGATGGTGTGCCGACTACAACCTCAACATACGGATAATTCACTAAATAAGTAA
CTTCCTCGTGGAATATCAcGGTCAGCCTTTGTTTTGGGTACACATTCAATGAACTGGTTTCCTATTAGTTTCTGCTTGTCAACAAGTTGTGTCATTT
ACTGATAAATATCCAGCCTTTTACTAAGGAATACCGATTTTCTCAAGTCTCAGTGTGTGGAGGGGGTGGGAGTTTGGTGGGGAAGCTTTTCAATTTG
AGAACCTTTGCCGTTTTTTGTTTCAATCGTGACTAAGATATAAAAGTCCTTGGTCACCACTGCCAGCTGCTACCTGTTCACTGCCTCCCTGACGGGT
AATGAGGCCATCCGGAGTTTTAGTGATTAAAATGTTTTATTACTTGTATTCACATCATCATTAAGAAAAThACTTCTAGTGGATCAATATGGCAAA
TGAGTAAACCAGAAGATGGCGTTGAAGATTAGCAATTACATTTAAAAACAAGGTAGTGGTGTGAAGGCTCAGAGTTTCACACAACGACTGTTGTTCT
GAACTAAAAGCAGAGGATGTGGATGAGTGAGGGGAGGGTATGTTGAAATGACTAAACCCGGTGCAGAGGACACGTATACGAAAGTGCAAAAAGCTC
TCATTTGCCATTTTTTGTCCTCAGCAAACTCCTCTCTGGTATGTTTGTGAACGTTTGAGGGAAAGGTAACACTGATTAAAATATAATGCAACAAGAA
GGCAACGATCTTTGCTTTGTTCACTGATGTGTTCCAAGTGCCTAGGACGGTTCCTGACAAGTAGCAGGTGCTCAATACATTTGTCAAGTGAATAA
ATCTGTGAAAGGAAAGGAAGAAAAACAGGTTGTGACCAGAACTTTTTGTGAACAAAACCCTGTTGTTTATTACATAGACTTTGGTAATGGAAAACTT
ACTTGGGTGAAATAAAAATGGCATCCTGTTAATTTCTGAGAACGTCTGTTGGTTTTAGGAAAAGCATACCTATCCATACCTCACTACCTGTTTTGC
TCTAGCCTAAACATAGATTCTTCTTTGGTTAGGAGACATTGACCCAATATATAAGATTAAAACAAAACTGAGAAATAAGTGAAATCAAAAGCTTTT
CAAAAGTTGCAGGTTGACCTACGGTGGTGGTGGTCTAAAATCATATAGAAACCAGAGATCTGATGGTTTACCTGAAAATGCGAAGTAAATGCACATA
CTCAGCATCTCAGACGATCGAAAGCTCAACGGGTGAAAGCTCAGGGGGTTAATTTTGCGATTAAGGACATCTTGGAAAAGTATGTAAAATCCCTGGT
CCACTTAAGTATTATTCCTATTTTGGGCTTTTATTTATTTATTTTGAGATGGAGTCTTGCTCTGTCGCCCATCTCGGCTCACTGCAACTTCAGCCTC
CCGGGTTCAAGCGATTCTCGTGGCTCGTCCTCTCAAGTAGCTGGGGCCACGCCCCGGCTAATTTTTGTATTTTTAGTAGAGATGGGGTTTCACCGTGT
TGGCCAGGCTGGTCTCGAACTCCTGACCTCAGGCGATCCACCGCCTCGGCCTCCCAAAGTGCTGGATTACAGGCTTGAGCCACAGCGCCTGGCCTA
TTTTGGGCTTTTTATACCCCACTGGTAAACTGCTTTCCTCCAGGTTGAGGTTAAAACGACATCATTTTAAGGTGAACTGAAGTCTGGAAGTGATTAAG
CACTTGGATCCTTAGGGAGCCTCTCCCCGCCCCCATCTCTTTCATGCTAAGATAATTAAAACTTCGGGGCCGGGGCATTGTCTGAGTCACTTCAATT
CACCAGCCTAATAGATGCAAAAGGATGTAAGCATGTCAGACACGCAGAGACAGTATTTGAATCAAGCTTAATAGCTCAAGGGAGCTGGGCCATTTCC
TATTTTCTTTTTAGTGAGTGCGATGTTCTCTAAACACCGCGGAGAGGCAGGGTTCCCGGTGATGGCCTTGCCGAGGGTGCTCCCGCAACCTCCACCT
CCAGTTCTCTTTGGACCATTCCTCCGTCTTCCGTTACACGCTCTGCAAAGCGAAGTCCCCTTCGCACCAGATTCCGCTACTACACGCCCCCCATTT
CCCGCCCTGGCCACATCGCTGCAGTTTAGTGATTGACTGGCCTCCTGAGGTCCTGGCGCAAAGGCGAGATTCGCATTTCGCACCTCGCCCTTCGCGG
GAAACGGCCCCAGTGACAGTCCCCGAAGCGGCGCGCGCCCGGCTGGAGGTGCGCTCTCCGGGCGCGGCGCGCGGAGGGTCGCCAAGGGCGCGGGAAC
CCCACCCCGGCCGCGGCAGCCCCCAGCCTTCACGCCGGCCCTGAGGCTCGCCCGCCCGGCCGGCCCCGGCTCTCGGCTTGCAAAGTCCCTCTCCCCA
GTCCAACCCCCGGCCCCCACAGGCCTCGGCGCCCCGCCCCGCCCCAGGCCCCGCCCCAGAGAGTTCTATAAAGTTCCTCTTTCCCACCTCGCACTCT
CAGTTTCACCGCTCGATCTTGGGACCCACCGCTGCCCTCAGCTCCGAGTCCAGGGCGAGGTAAGGGCTGGAGTCGGGCAGGAGGAGGGGTGTGAGGC
TGATACCAGAGAGGACCCGGAGCGCGAACCAGAGGTTCGACCTCCAGGGCAGCGCAGGGTACCCCGGCTTCGGAGCGGGAAGGGAGCGCGCCCCGTC
CTGGAGCTCCGACTCCCACCCCATCTGCGCTGAGCCGGAGGCGCGGGTTTGGGCTCCAAGGCCCGCCTCCTTGGCTCTGCCCGAGCCTCCCCGCCTG
CCCTCCGCGCTCCTGCGACGGGGTCGCCACAAGCTGGACGGGATGAGCTAACCGGACTGTCGGGGCCCCAGGAGTGGCTGAGGCGGGGCCGTCCAAG
GCACCCACACAAGACGGCACAACTGCCTGCGAGAAACAGGCCCGGCCCTGTGGACCCCAATCCTAGGCTCCTTCCCCTGCTCTTCGTTCCTAAGGGG
CCCAAGCTCACGGCGGCCTCCGGCGCGGTGCTCACCCGCTGGCGCAGGAGGAGGAGGAGCTCCACATTTGGGTCGCTCCGAGCCTTGCGTGCGGTGG
CNNNNNNNNNNNNNNNNNNNNCTCCGGGGTCCGGCGGACGCTCTGCGCGCGGAATCCCCCGTACTGGGGCTGCAGCCCCCGCGTCTGCGCCACTTGT
CGTTTGCAGAGCCCACTTAGTGCGCGCTAGCTGGGCAGGGATAGGGGTCCTATTCGGGGCGAAGGGTCTGGATGCGAGCAGAGAAAGCGGAGGGTGG
AGGAACCCGGGGCTGCGCCCCTGGAACGCCCGGCCGCAGGCGAGGTCCTCCGCGCGTGGAGGCCGCCAGGGGAGTGGAAACTGACAGAGTOGCCGGA
AGGGGCGAGAAGCGGGT1GGGAGTGAGCGAAGGCAAGCGAGAGCTGCGAGTGAGTGCGGAAGGAGGGCCAGGAGGGGTGGCGGCTGGGTGGGGAGAG
AGGGTGCAAGACGAGCGGCGCGTGTCGGGAGCCTTTGGGCTGCGGGTGCGTTACAGGAGAGCAGGCGGGTAGGAGCCTTCGCGGGGGCCGAGCTCGG
AAGGCGGACGGCTGTGCCCGCCCAGGGATGCGCCCGGGCCGCCGCGAAGGTGCCTTCTTCCGGGGGCCCGGACGACCCTGACACGGCACGCGCGC
GCTTCGCAGCCTCAAAGACTCCGGGGCCTCGTGGTCACTGGCGCAGGGGATCGGGGCGGGGTGCCCGGAGTGCGGTGCCTCGTGGCTGAAGGGCAGC
TCTTCTCCCCGCAGTGCAGAGCAGAGCGGGCGGAGGACCCCGGGCGCGGGCGCGGACGGCACGCGGGGCATGAACCTGGAGGGCGGCGGCCGAGGCG
GAGAGTTCGGCATGAGCGCGGTGAGCTGCGGCAACGGGAAGCTCCGCCAGTGGCTGATCGACCAGATCGACAGCGGCAAGTACCCCGGGCTGGTGTG
GGAGAACGAGGAGAAGAGCATCTTCCGCATCCCCTGGAAGCACGCGGGCAAGCAGGACTACAACCGCGAGGAGGACGCCGCGCTCTTCAAGGTCTCC
GGCCTCGGGAGCCGGCGGGGGCGCGCCGGGGAGGGCCCAGAGACAGAGCCCGGGGTCCCCGGCGCCGCCTCCGAGGCGAGCCCAGGGGACCGCGCGG
GGCGGACGGGCGGGCGGCGGAGGCATCAGGTGGCGTCGCCGGAGCCGCAGGAGGAGGAAAGGAGGCCTCGGCTCTCAGCGGGACCGCGGGGGCCGGG
AGCCGGGTCCTGGGCGCGTGGAGGCTGCAGGGAAACCGCTGAAGGCCCGGCCGGGCCCGGGGAAGGGCGGCCAAAGGCTTGAGGGGTTTTGCGCGTT
TCCGCGCCTGTGCCGGCGGCTGTTTTCGTCTCTCACCGCGTCTCTGTTTCTCTTTTCGCTGCTTTTCTCTCTGAGTCTCTCTCTCTCCATGTTTTTC
CTGAGGTCAGCCTCTCTTCTCGCTCCTGCTAGCTCTCTGCGGGTACTCCCACCTCTGTCTTTCTCTTTGTGTGTCTCTGTCTCTCTCTTTCCCCCAT
CGCAGTGGAACTCAGGGCCTCTGTCTAGAGCTGTCTCCCTTGCCCTTTGCGCGAGTGCACACACGTGTGTCGTTGTTACGATTGTTCTCCCCTAAGG
CAGTTTACCCAGAGAACTACGTGTCTGGGCCCAGCCCCCACCTGTGGGCAGAGCAGGGGAAGGGGACTTCCTCCGGGAATTTGGTCTCAATTTGCTC
TCAGAGTGCCTCAGCTGTGCTGCCATCCAGATGTCTCC2GTGGGTGACAGCTCACACCACAGCTGTCCTTAGTCCTAGGCAAGCTCACTCAGACACT
GGGTGGGTAGAGCCCCCTGAAGAACCCCGCATCTCACTCTACCGGTTATAAATACCCAGAAAATGTGTCTTCAACTTGGCAGTGATAGGGTCCAA
GATGGAAAATCATTTTCCTAGAGCATAGCTGGGGTCTTTCAGTTTACGTTCTGAGCAACGGTGTAAATCTGAAGGACCTATGCGCCATTCTTTCTTT
TAGAAATACAATTTCAAGATTCCTTATAAAATATCTGTATTTTGGGTTTAGCATGGTGGCTCACACCTGTAATCCCAGCACTTTGGGAAGATGAGGT
GGAAGGATGCCTATGCAACATAGTGAGACCCTGCCTCTGCAAAAAATAATAATTAAAAAAATTAGCCAGGCATGGTGGTGAACACCTGTAGGCCGAG
GGCCCAGCTACTCTAGAGGCTGAGATAGGAGGACCCTAGAACTCAGGAGTTCGATGCTGCGGTGAGCTTTCATCGTGCCACTGTACTCTAGCCTGGG
CAGCAGAGCAGGACTCTGATTCCAAAAAGAAAGCTAATAAACCAGACATGTATTTTGACTTTTCGTTCTCTTCATTCTTTCCCACCAGGCTTGGGCA
CTGTTTAAAGGAAAGTTCCGAGAAGGCATCGACAAGCCGGACCCTCCCACCTGGAAGACGCGCCTGCGGTGCGCTTTGAACAAGAGCAATCACTTTG
AGGAACTGGTTGAGCGGAGCCAGCTGGACATCTCAGACCCGTACAAAGTGTACAGGATTGTTCCTGAGGGAGCCAAAAAAGGTAGGGGCTCTCCTGA
ATTTGGGTCACCTAACAGAGGCAGCCAGATCCTTGAGGCACCTTAACTTCATTCTGAGCATCACTTTCTAGCTTTCCTTTTGTATTGCCTGCCTGCC
TGCCTTCTGCCTCACAGTGAAGCCCAGCCTCCTTGCCTGAGTTATGTGGGTCACAAGTTGAAATTCCTGTGGGGTTCAGCATAGGAGAATTAATCAC
ACTGTATGCCTCAATGTATATGGGGGGGGGTGCATTGAATATGTGTTTTCCAGTTAGTCTTTCAAAAAAAAAAAGAAAAAAGAAAACTGGATAACTT
GTTAAAATTTACTTATCTAATAGTCTTTAGTGAGTGACTATTTTGCAGAAAAGGAGTAATGAGAGGAATATCTCATCACTATGCATGGTAATAAAAC
TGTCAACTTTTAAAGTGTAACATGGTCAGCTGAGGTGTTTATTAAAAGTCAGATTCCTGGACCCCTCTTTTCAAGATTCTGACCAGTGTGTCTGGAA
GAGAGCTCAGGAATCTGCATCTGGAACAAATCCTTCAGGTGACCTCACTTGGGAAACATCTGGCTGGCGGGGTCCTTTATGTCACAAGGCACATTCG
CATGCCATCTGTCATGTATTTCTTTACCTGTCCTGTACAACTCAGACCTTGTAGAAAAAAATCAGTTTAATAGCATGAAAAAATAACTTCCCAAGGG
AGTTGCTGAAAAATACCTTATTTGTCAACACCGTGTTATGCATTCTAAGAATTAAATGCTCAGGTATTTTTACAAGATTTGACATTTAGTTAGGTCA
GTTCCTGTTTTTACGTTGTGCCATTTCCCTTTTCCCCAAACATGTAGGAGCCAAGCAGCTCACCCTGGAGGACCCGCAGATGTCCATGAGCCACCCC
TACACCATGACAACGCCTTACCCTTCGCTCCCAGCCCAGGTATGGTGGAGGGCACTGGGCTCCCTGAGGGCGAGGCTGTGTGGGCCAGCTGCCCACA
TGGCCAGAGAACCACAGCAGCCCAGACAGCAGAACTTGCCATTTGCTATGGCTGCTCCAACAGCCCAGAAAAACCCCAGGTCACTGAACGAATGTCT
CACTTTCCACACGGTGCTGCCATTGGTGTGGATTTTAAGTTGGGGAGGGTCGGGCGTGTCCGCCTGTTGGAATATGCTTCTCAGGTCTTCTGGGAAA
CAGATGTTTTGTGGAGTGGAAGATTTTGGAAGTAGTGCCTTATCATGTGAACCACAGGGCAGCTGATCTCTTCAGGCTTTCTTGATGTGAATGAC
AGCTTTGTTTCATCCACTTTGGTGGGTAAAAGAAGGCAAATTCCCCTGTGGTACTTTTGGTGCCAGGTTTAGCCATATGACGAAGCTTTACATAAAA
CAGTACAAGTATCTCCATTGTCCTTTATGATCCTCCATGAGTGTTTTCACTTAGTCTGATGAAGGGTTCACTCCAGTCTTTTCGGATGATAAAATGC
TTCGGCTGTCAGTCTATAGGGATTCCCTGAGGAGTTTGGAGGCTGTAAGAGCACCCCCCGTCTCAATGCCAGTGCTTCTTATCTCAGCCTCTCCT
GCACTCCTTTACCCCCGTCTCGATGCCAGTGCTTCTTATCTCAGCCTCTCCTGCACTCCTTTACCCCCGTCTCAATGCCAGTGCTTCCTATCTCAGC
CTCTCCTGCACTCCTTThCCCCCGTCTCGATGCCAGTGCTTCTTATCTCAGCCTCTCCTGCACTCCTTTACCCCCGTCTCAATGCCAGTGCTTCCTA
TCTCAGCCTCCTGCCTCCTTTACCCCCGTCTCATGCCAGTGCTTCCTATCTCAGCCTCTCCTGCACTCCTTTACCCCCGTCTCGATGCCAGTG
CTTCTTATCTCAGCCTCTCCTGCACTCCTTTACCCCCGTCTCGATGCCAGTGCTTCTTATCTCAGCCTCTCCTGCACTCCTTTACCCCCGTCTCAAT
GCCAGTGCTTCCTATCTCAGCCTCCCCGTCTCATGCCAGTGCTTCTTATCTCAGCCTCTCCTGCACTCCTTTAGCAGGTTCACAACTACATGATGC
CACCCCTCGACCGAAGCTGGAGGGACTACGTCCCGGATCAGCCACACCCGGAAATCCCGTACCAATGTCCCATGACGTTTGGACCCCGCGGCCACCA
CTGGCAAGGCCCAGCTTGTGAAAATGGTAAGGAGGATACCAGTGCAGGAAATAGAAGAGCTAACTGCTAATGTGGCCATGGGCCATGGCGAATCCTG
GTCTGTCCTGGGCAGCACCAAGCTCTTTCCCCTTCTTAGAGGCTGCGTGTGCAGCTCGGGGAGAGGGGGGTTTTCTCACTCCTGTGGGATGGTGGC
ATCCCACAGCCAAGTTTACTCTCAGGATCCATGCTAGGCACTGCCCTTCGTGGGATCTTATTTAAACACCACAGAGATCACTCCAGGCAGTGGAAAA
CAGCCCCCCACTTTTTTATATAGAGAGGAGATTGTGGAAGCTCATAAATAATAAGTAGGGATGCTGGGAGATTATTGAGCTGGTATATTTCTCTCGAA
TAGTGGTGTGTTCATTGTTGCTATTGTTGGAATTAGAGGTGATACTAAAAAGGAACGGAAAAATACCATGGCAAACACATAAAACGTCTCTACAGG
TGCCTACCTACCACTGGGTCCCAGAAAGACTTGCTGATTGTGGATTAGATGTGGTCCCCTGCCCTCGGGAGTTCCCCAGACATCAAAATGAGCTCCC
TCAGCAGAAGCTCTCATTAGCTGGGTGCAGGGAGAATCTAGCAATGCGGTAACTCAGGCCTTCAGGACTTTTGAGTTAGTTACTTAAATGGAAGTCC
TGAGACAGGTCAACACACTGAACCCTTTGGATGTTTTAGGAATATTTGTTCCTTTGCAATTTGGCCTTTTTTTCCCCAGTCATGTTCAGGTTATCCT
TGGCTGCAGGTAGTGAAGAGGTGTACCACTCTTTAATGTTTGTACTGAGTGGCATGTTCACAAAAAGCTCAGAATTCAGTTGCATTGGCTTTTCAA
GAGGGATTTGCAGCAAGGGCTGGTTTCCAATATGGTAGTGGAGGAATTGGCTAACATAAAACTAGCTCCTGAAATTCAGACTTGCGTTTACTGCTCT
GGCTCTATGGAATTAGTAACCATGGGAAAGAGGCAATGCTTAGAATTGTGTAAAGTGATGGAGTAGGTAGAGACCTGTCTTGAAGCATCGGTAACAG
TAATGAAGCAGCAGCAAGATCAGGTGTTCTGTGCCACATTCTTTTTCCATTCATGTTTCATACAACATTGTCTTCAGGAACATGGTTCAGTGTTCT
CCCAACCCACAAGAGACAAGCAAAATAAAAATGGACTAAATAATTTGCTAGTTTTAATTGGGCTCTTTGGCACAGACTCAAGTAGACCTGAACTCT
CAGCTCTGTTGTGAACCCGCTTTGTGAACAAGGACAAGCATGTCATCTGTCTCTGAGCCTCAGTCTCCCTGTTAGTCAGTTACAGAGAGCGCTC
CTGATCTCAGGAGACAGGCCAGCAGAGTGCTTCCCTGTGAGTCTGTGTTCCTAACTTAACAATATCTCAATATTTTGGGGAGAAATATTTTGGTT
TCCTTCAGTCCCCCCTTGAAGCTTTACTTTCAGAAAGTTTCACATGTTAAAAGCTAAGTTGGTAGTTTTGGAGAGATTTGGGTTAGAAGTTTTAAGA
TAAGAGATTTGGCAAGGAGGAGGAGAACATAGTTTGGAACAAGCAGAAAGGAACCAATTTGGGTACATTGTTCCATCTCTTATTGTATCAGTCTTT
CAGTCCTGAAAATAGGTCAGTTCCGTTAAATCATTGCATCCCATTTCAGAGGTGGTGTAAGTGGGCTTCCATAAGAGTTACGTCTTCCATATGGTGT
AGGCACACAGGTACTTTATCAGAGACATTTGTATGGAAATAAAAAGAAAACAAAGGTCTATGTTTCAAGCAGTCTATAAGCTAGTTTCTGGAGAGCA
ACCAGCAGATTTCTAGATTTGGGTTCAAATAATTTTAAGTTGGAGTGGACAGGCAGTGGCAATCTGACAGATTTTTCTGGATTATAGTTTGCATC
AGGCACGGTTTTAATTCCTAGTGATTTCAAGTCAGAAAGGTGGGAGAAAGTTGGGAATGTTAGTCTGAAGAGCCATAGCAGATATTAGAGGAAACTA
AAACTTCAGGATTCAGTCCAGATAATTTTTAAAAACTCTAAAACAATGGACAGGGCTAGAATCTAATAACAGCTATGCTATAGCTGTTCTGAAACAT
TTTTTCTCTCTACAGTCACCTCCCTGTTTACCAAAGATAATCACAATAAGTCCAGTTTACTThCAAAACAAGTTTAGTGTCATTATATTTAACCGGA
TCATTTACATAAGTACAGCAAGAACAGTGATTTAACATATAAGCTCTTTTTTAAGTCAGCGTTGCTGGAACTTTTCGTAAAGAATTTTATTAGATTAG
AACACTAAAACCCTCTTCAGACTAGQAAGCCGTGCCACAGACTTACCAACAGATTTTATCTGAGATACCTGGGTAAATTTTTTTCTTGAGGTCTCCA
AATGTTTTGAGGTTCCTGGGCTTGTCAGAAAATGACATTATTTACTTACTGCAAGGCTAGAAGTGCTGGAAGGGCACCGCATAGTTCAGGAACAAA
GTGCCAGGCCAGTTCTTTCAAGGGTTTTATTGGCTCCTTAAAGTTAACGTTCATTCCTTAAAGCAGTCTGGTCACATCTGAAAAAATGACGTCCCAG
TCATAGCCTTGATGATATAACCTGTGTTTCCAATTTTTCCAGTCCGAAGAGACCAGATTCCTAGTGAACTTATGCAAATAACTATCTTGCCATAAAA
ATAGAATTTCTCACATACAGTTTCTAAATCCTGGAGGGCTCAGGTAAAGAAAAAGATAAATGTTTCAATATTTGTGCACAAAAATATACTTTACAAA
ATTACTGTAAAGCTGTAGATTACTTAAGAGGGAAAAAAAGTTTTCTCAACTATGGAAAGAAAACATAAAAAGAATCAGCAATGCTTTAAACAAAAAA
TGTTGCAAAAAATCATTTTAGTCCTTCCTTGGTTTAGTTTCGTGTAATTCTTGTTTTGCTTGATTTGGGGTTACTAATTTTATGAGTCCAGTTTTTT
TTTCATTAGCATTCTGGGAATTTTTAATGACTCCAATAATATGGTCTGAAAGTTGTCAGGAACCTGTATTCAAGAGTACTTCTTTCAGTCCTTTCTA
TGAACCTTCTTGAAGACATAACACTTThGGATCTGCAAAGAGCTTTTAGAAAAAGTATCATAATAAAGCAATTAATAGCAGATAACAAGACTTAAAA
TGGACATAGTTAAAGACACAATTGACATGGAAATTTGGTTACTTTCTGTGGCTTACAACAATTTAACATAATACCCATAATTATAACTGACAACATT
ACCAAAACATATCAGATTTTTAGGAATCTCATACAATTTTAGAACACATATTAATAACATATCCAThTGATATAGTTTGGATCTGAGTCCCCACCCA
AATCTCATGTTGAAATGTAATCCCCAATGCCAGAGGAGAGGCCTGGTAGAAGGTACTGGATCATTGGGGTGGTCCCTCATGAGTGGTTTAGCACCA
TCCTCTCAGTACTGCTCTCATGATGGTGAGTGAGTGCTCTCAAGATCTGGTTATTTAAAAGTGTGTAGCACCTCCCCCACTTCCTCCTTCTGCTCCT
GCCATATAAATATGCTTGGTTCCCCTTCACCATCCACTATGATTGTATGTTTACTGAGGCCTTCCCAGAAGCCAAGCAGATGCTGCCATGCTTCCT
GTACAGCTGGCAGAACTGTGAGCTAATTAAACCTCTTTTCTTTATAAATTACCCAGTCTCAGGTATTTATTTATGAAGTGCAAGAATGGAGTAATAT
AGAAAATTGGTACCAGTAGTGGGATATTGTGATAAAGATACCTGAAAATGTGGAAGTGGGTTTGGAACTGGATAACAGACAGAGATTGGAAGAGTGT
GGAGGCTCAGAAGAAGACAGGAAGATAAGGGAAAATTTGGAACTCCCGAAGGACTTGCTGAATGATTGTGACCAAAATGCTGATAGTGATATAGACA
ATGAAGGCCAGGCTGAGGAGGTCTCAGATGGAAATGAGGAACTTATTGGGAACTGGAGTAATGGTCGCTTTCGTTACGCATTAGCAAAGAGCCTCTA
CTCTAAASATTTGTGGAACTTTGAACTTGGGAGCAATGATTTAGGGTATTTGGCAGAAGAAACTTCTAAGCACTGAAGTCTTTAAGATGTGACCTGG
CTGCTTCTAACAGCTTATGCTTATATGTGTGAACAAAGAAATAACCTGAAACTGCAACTTATATTTAAAATGGAAGCAGAGCATAAAAGTTTGAAAA
ATTTGCAGCCTGGTCATGTGAThAAGAAAGGAAAATTTCACTAGCACCTCTATCTAGGGTCTCAGGTTCTCATCTGACAAAGTACACAAAGGCCTTA
CAACCCCATGTGTCCATAGATAGAAGAATAAAAAAAGACAGTCTGTAAGAGAGGAAATCAAAAGCTGTTCATGGTAGTAGAAAGGATTACCAATGGG
TACCACAAAAAGTCAAGACTCATATAAATAATTAAAAACAAGGCATTTACACTAATTTGTATAAATGTTTCTCTCCCAAGCTAAGGTATTTACTAAG
GAAATCAATTTGGTGGTGGGTCTAAAGAACTTTATTTAACTCTGTGTCAAATCCAATCTCTGCTTGAATGCTGCTTAATTAATTCCCTGAATAACAT
TTCAAAAACATGGTAAGATTAACATCTTCCATGGGCTGAGAAAGGCTTAGAGAATTAGGCTGAAGAGTGTTTTGTCAATCTTGCAATGACTTACAGG
CATTTGAAAAGTGAACTTCTCTAAAAGAAATTTATTTTAAATATAGCCAGTCTAATTTATTTCAAAGTGATGTAAACCAATAAGCCAAAATAAAAAC
ACCAGAGTCACTATATCCAAGAGCCAATTTATACAAATAATTTTCTCCTATTTAAATTTGGAAATGAAGGAAAAAAGAAAAAACTCTTACCCTCCCC
TCTCAACTGAGCACTAAAAATAGAGATGTGAAGAGCTGACGTTGGTAAGAAACCTTACCCTTTTCTGCTGGTTTCTTTCAGATCTCTCAGAGCTCCA
TCCATTGGCCCTAGTGTGAGCAAAGTGTGCCTTTGTGGTTGTCAAATTGAAGGAAAGAAAATATGATTTATTTTCTCAATCCTCATATTTTCTTACC
TGAGACACTTTCCTGAAAAAAAAAAGATTAACAAGAGAAAAGCCAGAAGAAGTTTATTGATGAATATTGTACCCATCACCTAGAAGAAGGCCTCAGT
TCAAATGTATTTCTCTCCCAGGGCAGAGTAGATGAGGAATCTTTCTTAAGTAGTATTTTAACAAAAAACAATAAATCTTAGCATAGAGACAAAGCAA
AGGAGGGGGCAGTTCCAGTCTTTTAAAAACTGGGAAAATGTGGGAAGATAGTAAACCTTGTTCCCAGATTGTTCTGGTGCTTGCTGATGCCTTCTCT
GGGCCAATTTCTAGTAAGGAAGGATTTATGTCCTGACATCAGGCAAATAGAGGTGGGGCAGAGTGTTTCCTTGCATTTTCAGTGTGTTTAACTTAAT
AATCCTCAATATTTTTGGAAGAAGTTTTTTGGTTTCCTTCATAACGAAATAGAAATAATGGACAAATAAACAATCTTTCTTCTTCTTGGGGCTCCAT
TTGCTCAACAATACAGGTGGACTCAAGTCAGTATACATGCATAGGCAAGTTTTTCTTCAAATAATATATCAACTGGTTATATCTAAAAGGGTATTC
CAAAGGGTTTTGAGCCCTTGAGATTGCTCTGAAGTAATTTTATGTATCTCTAAGGTACCAGTGTTTATTCTCGTAAACTGTTCTGTAACTGCAGCAC
AAGTTTGGCTGTATTGGATTTGATTTCCTAAAAACAATGATCACCTGACAAACACGCTTGTTGCAGTGTGCCACCAGTACTCCCCTAGGCCAGAAAG
ACACGTTGGGTCCTGTCTCATCTCTCTGCAAAGGAGCTCTGGACAGGCTATACCTAAATTAACACAGGCAGACATATATTCTCTGTTTTAGTGTTGC
ATTATGAATTATATGGTGTTTGAAGGCAACCCGGCCTTTTCAGTGACCCCTCCTCCTGCCACACTGGTAGTGTGTTGGCAAAATGTCTGTCCTTAGG
GAGTCCTCAAAGACTCCTTCCTTCTCTTCCCTCTGCCCTCTGCCTCTGCTCCCCTGGCTAGTCTGCGCCCACAGGTCTTGGCTGTGGCACACTCCCC
TTCTGCAGGCATTGCTTCCCTTATTCCACCCTCTGTTTGGGTCTCCAAAGTGTGATATTTGTCTTGAGAAACACTTTGCCCCACTATGTCATTCCCC
TTTCCTTACTCTCCAAGCAGCTGGGTCACTGGTGCTCAGGACTCAAACTGGCTGGGAATGTGAGAAAAGCTCCTTTTCCCTCCTGGCCTCCTACTTG
AGACTAAAGCAAAATGCTGAGGGTTGAGGAAGGGAGAGCGAGTGCTCGCTGAGAAAGGAAGAAATGAGTGTTTAGACTTCAGCCACTCCCTGATAAA
GGGGAACGATCTTCTGCCTCCTGGGAAAAACTTTGATGAGACAGCAAACTTGAGAAAAGTTTGTGGGAAGGAGCAGTGAGAACCGCAGGTTACGAAG
GGGCCAGGTGGGCAGGACTGAAGGATAACTCCATGGTAACCGGCGTGGATCGATGACTATGGGGCCAAAGAGTGGGCATGCCTGGGTGTGTCTGAGC
AGAAACTGCATTGAGGACAAGATCCCCTGCCCACCAGCACAGGCTCTGGCTCTGTGGGAGCTATGCCAAACTTAGCCCCAGTTCAAGGGAAGGAGAG
GGAGGAGAGGGAGGAGAGGAGGAGAGGGATCCAAGTGAACTTCTTGAATTTGCATCACCCTAGTGGAATATGAGCTTAAAATTAACAATTATA
TTGTTAAAAGAAAATATGAACTACATGTCTTAAAAACTTTGGCTCTGGACTTAGATTTCTACGCACCATGTCCGCAGCATAACACTGCCCATTCTTTA
TTGTAATTGTCTGTTTATTAATTTGTCTTCTGTACTATAAATTCCTTGAGGAAGGGAACTGTGCTTTCCAGTAAACACCCAGTACTGGTGCAGCACC
TGGCTTATAGTCAGCACCATCGAATCCTCAGGAAATGACACAGTTGGCGAACCTAGATGTCTACCATGTCCAACAGTGTCCAATAGGATGCTGGCCA
AGTGATGAGCCACCTATGGCATGAAATGCTATAGAACCACTTACAAATTGTGCAGTATACAAGTATTTCATGGCATGGGGAAATGGTTGTTACTTTG
TAAAGTAAAAAGAAACAGGTTTCAAAACAATGTGATATCTTTTTAAAATTTCATGGAGCTCATACATAGAATTTTTAAAAAGAACATTCAACAGTG
TAAATTATACCCCTGAGATGTGGGGTCATGGAAATTTTTTATTTTCTTGTGTGCTTTTTTGTACTTTTCTCCYGTTTCTCCTGAGACATATTTTG
TTACTAGAAAGGGAGAAAAGCACAAATGTAATCTTTCTAAGTTGATACGTCCCTTTATATTTTTGGTGGGCTTCCAGTTTCTCAAGCTGCCTTGTG
GGGTCCCCAGAGCTCTGGGGGTGGGACCAGCTCTGAGGTGAGGGACCAGGGGCTCGGTCTCAGGGCCCATCTCCCAGTCCTGCCTCCATTAATT
TCTTTTCCTACCATTTCCCCTTGATATGCTTTCCCCCACCTCATCTTTCCCAAGATGCTTCTTTGTCCTCTGAGCTATGACACTGAGGATGAACGCT
CTCTCAGCTTTCTGGCTGCAGCTGCCTCCATGCAGGGACCCCCAGCCTGTGAACTTCCCTCTGGCACCCCCACTCCCCCATGCGGAAACTCCCTCGG
CCACCCGTTCTCATCCCCAACCCAAGCGGGACCTCCCTCCCCACCAGCACCCCCATGCGGGACCTCTCTCCCCCACCAGCACCCCCATGTGGNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNCCCCCTCCCACCCCCATGTGGGGATCTCCCTCCATCCCTAGCACCCCATTGTGGGATCTC
CTTTCCTCACCTCCAACCCCCATGTGAGAACCTCCCTCTCACTCTCACCCCCATTCTGGGACCCTCCTGAGTCTCTCCCTTCCTCAGTCATCACAG
TCTCTGTACAGCTAGGGACTGACTCTTTGCCTTTGGCCCTGCTGTTTCCCCTCACGCTAATGTCAGCATTTGGGGTTGCCTGGAAACAGGAAGGATA
AGAACATTGATTCTTCTTTTTTATTTGTGGAAAATATGCATAACATAAAATTTACCATCTTAACCATGTTTAGGTATACAGTTCAGTGGCATTAAGT
AATTCACATTGCTGTGCAGCCATATCCAGCATCTGTCTGCAGGACTTTCTCATCTTCCCAAATTGAAACCCTGTCTCCATCCCACACTATCTCCCCA
TTCCCTGGCCCCTTTGCCCCCAGCCCCCACCATTCTACTTTCTATCTCTATGAAACTGACTACTCTGGATACCTTGTATGCGTGGAATCCTGCAGTA
TTTGTCCTTCTGTGTCTGGCTTATTTCACTGAGCTTGATGTCCTCCAGGTTCGTTCATGTTGCAGCACGTGTCAGAATTTCCTTTCTTTCTAAGGCT
AAATCATATTCCATTGCATGGGTGGACCATATTTTGTTCATTTATCTGTCCTGAACACTTGAGTTGCTTCCGTGTTTTAGCTACTGTGAATAACGCT
GTTGTGGACATGGGTGGACAAATGGGTCTTCAAGACCCTGCTTTCAATTCTTTCATGTGTATACCCAGAAGTGGAACTGCTGGAGCGATGAAAATTC
TATAATTAATATTTTAAAGGTACCACTGTACTGTTTACCACAGTGGCTGCATCATTTTACATTCCCACCAGCAATGCACAAGGATTCCAGTTTCCCC
ACATCCTCACCAACACTTGTTATTTTCTGAAGAACATTGATTTTTTTAAAAAGGATTCATTCCACAAACATCTTCTGAAGACTCCCCATGTTCCTGA
CCCTCCCTGCTTTCCAGGGATAGCCAGCTGTAATCGCACAGCTTCTGTGTGGGTGCAGGAGGCCAGCAAGCACTGACACAGACGCTACGGGCAGGGC
ATTTCCGAGAAGGAAATATTTTCATTCTCCATTTTTAAGGTGAGAAACTGAGGCCCAGGAGTGTAAAGCCAGTACCCTAATATACAACTACTAAAAG
AGGAAGCCAGGATTCAAATCCAGGCCTTTGTGGTTTCATAGCCTTATCTCTTAATTCTGATGCCAGCCTGCCTTCCCTICCACCATTGACGTACCAA
GAGTCAGAGAGGTGGCACGGIYGTACCTGTATCCACACCACTGGTGAGCAGCCCTCCAGCATCACACCCAGACTTTCTAAGATGCTTGGTATCCCACA
GATGGGAGCCAATTTCTTTAGGTGGGGAAAGAGAATGGGAGCAGAGAGAACAGCCTGTTCCACAAAGAAAGGAGAAGAACCAAGAGTTTGGGATTAA
GCATAAAGGTCTAATTAACATTTATTAGGTGCTAAGTGCACTGCCATGTGTCAACTCCTGTTATCCTTGAAGTAATTTGGGAGACAGGCATTTCAT
TTCTCACCTTACAGATGGGGGCACAGAGCACAGGGAGGATCTTGAGAGGGTCCCATGGTCAGCAAGTGTTGGTGAAAGGATATCATCCTGGCTGGTC
CTACCCAGGTGCTCCATCTTCATCCTATAAAACTCTCTGTGCTTTAGTTTCTTCTATGAAACAGGACACCTATTTCATAGGGTAGTTGTCGGGATTA
GTGATAAAGTGCTAAGAGTAATAGCTGGGGCACAGTTAATCCTATGTAAATATTGGCGAATTGTCTTAGTCTGTTGCCTGTTGCTATAATGGAATAC
CATAGAGTGGGTAATTTACAAAGAAAAGTATGTATTTTTATGATTCTGGAGGCTGGGAAGTCCAAGAGCATGGCACTGGTACCTGGTAAGGACCTCC
TTGCTGC
HUMAN SEQUENCE-mRNA
CGCTCGATCTTGGGACCCACCGCTGCCCTCAGCTCCGAGTCCAGGGCGAGTGCAGAGCACAGCGGGCGGAGGACCCCGGGCGCGGGCGCGGACGGCA
CGCGGGGCATSAACCTGGAGGGCGGCGGCCGAGGCGGAGAGTTCGGCATGAGCGCGGTGAGCTGCGGCAACGGGAAGCTCCGCCAGTGGCTGATCGA
CCAGATCGACAGCGGCAAGTACCCCGGGCTGGTGTGGGAGAACGAGGAGAAGAGCATCTTCCGCATCCCCTGGAAGCACGCGGGCAAGCAGGACTAC
AACCGCGAGGAGGACGCCGCGCTCTTCAAGGCTTGGGCACTGTTTAAAGGAAAGTTCCGAGAAGGCATCGACAAGCCGGACCCTCCCACCTGGAAGA
CGCGCCTGCGGTGCGCTTTGAACAAGAGCAATGACTTTGAGGAACTGGTTGAGCGGAGCCAGCTGGACATCTCAGACCCGTACAAAGTGTACAGGAT
TGTTCCTGAGGGAGCCAAAAAAGGAGCCAAGCAGCTCACCCTGGAGGACCCGCAGATGTCCATGAGCCACCCCTACACCANGACAACGCCTTACCCT
TCGCTCCCAGCCCAGCAGGTTCACAACTACATGATGCCACCCCTCGACCGAAGCTGGAGGGACTACGTCCCGGATCAGCCACACCCGGAAATCCCGT
ACCAATGTCCCATGACGTTTGGACCCCGCGGCCACCACTGGCAAGGCCCAGCTTGTGAAAATGGTTGCCAGGTGACAGGAACCTTTTATGCTTGTGC
CCCACCTGAGTCCCAGGCTCCCGGAGTCCCCACAGAGCCAAGCATAAGGTCTGCCGAAGCCTTGGCGTTcTCAGACTGCCGGCTGCACATCTGCCTG
TACTACCGGGAAATCCTCGTGAAGGAGCTGACCACGTCCAGCCCCGAGGGCTGCCGGATCTCCCATGGACATACGTATGACGCCAGCAACCTGGACC
AGGTCCTGTTCCCCTACCCAGAGGACAATGGCCACAGGAAAAACATTGAGAACCTGCTGAGCCACCTGGAGAGGGGCGTGGTCCTCTGGATGGCCCC
CGACGGGCTCTATGCGAAAAGACTGTGCCAGAGCACGATCTACTGGGACGGGCCCCTGGCGCTGTGCAACGACCGGCCCAACAAACTGGAGAGAGAC
CAGACCTGCAAGCTCTTTGACACACAGCAGTTCTTGTCAGAGCTGCAAGCGTTTGCTCACCACGGCCGCTCCCTGCCAAGATTCCAGGTGACTCTAT
GCTTTGGAGAGGAGTTTCCAGACCCTCAGAGGCAAAGAAAGCTCATCACAGCTCACGTAGAACCTCTGCTAGCCAGACAACTATATTATTTTGCTCA
ACAAAACAGTGGACATTTCCTGAGGGGCTACGATTTACCAGAACACATCAGCAATCCAGAAGATTACCACAGATCTATCCGCCATTCCTCTATTCAA
GAATGAAAAATGTCAAGATGAGTGGTTTTCTTTTTCCTTTTTTTTTTTTTTTTTTTTGATACGGAGATACGGGGTCTTGCTCTGTCTCCCAGGCTGG
AGTGCAGTGACACAATCTCAGCTCACTGTGACCTCCGCCTCCTGGGTTCAAGAGACTCTCCTGCCTCAGCCTCCCTGGTAGCTGGGATTACAGGTGT
GAGCCACTGCACCCACCCAAGACAAGTGATTTTCATTGTAAATATTTGACTTTAGTGAAAGCGTCCAATTGACTGCCCTCTTACTGTTTTGAGGAAC
TCAGAAGTGGAGATTTCAGTTCAGCGGTTGAGGAGAATTGCGGCGAGACAAGCATGGAAAATCAGTGACATCTGATTGGCAGATGAGCTTATTTCAA
AAGGAAGGGTGGCTTTGCATTTTCTTGTGTTCTGTAGACTGCCATCATTGATGATCACTGTGAAAATTGACCAAGTGATGTGTTTACATTTACTGAA
ATGCGCTCTTTAATTTGTTGTAGATTAGGTCTTGCTGGAAGACAGAGAAAACTTGCCTTTCAGTATTGACACTGACTAGAGTGATGACTGCTTGTAG
GTATGTCTGTGCCATTTCTCAGGGAAGTAAGATGTAAATTGAAGAAGCCTCACACGTAAAAGAAATGTATTAATGTATGTAGGAGCTGCAGTTCTTG
TGGAAGACACTTGCTGAGTGAAGGAAATGAATCTTTGACTGAAGCCGTGCCTGTAGCCTTGGGGAGGCCCATCCCCCACCTGCCAGCGGTTTCCTGG
TGTGGGTCCCTCTGCCCCACCCTCCTTCCCATTGGCTTTCTCTCCTTGGCCTTTCCTGGAAGCCAGTTAGTAAACTTCCTATTTTCTTGAGTCAAAA
AACATGAQCGCTACTCTTGGATGGGACATTTTTGTCTGTCCTACAATCTAGTAATGTCTAAGTAATGGTTAAGTTTTCTTGTTTCTGCATCTTTTTG
ACCCTCATTCTTTAGAGATGCTAAAATTCTTCGCATAAAGAAGAAGAAATTAAGGAACATAAATCTTAATACTTGAACTGTTGCCCTTCTGTCCAAG
TACTTAACTATCTGTTCCCTTCCTCTGTGCCACGCTCCTCTGTTTGTTTGGCTGTCCAGCGATCAGCCATGGCGACACTAAGGAGGAGGAGCCGGG
GACTCCCAGGCTGGAGAGUCTGCCAGGACCCACCACTGGAAGCAGGATGGAGCTGACTACGGAACTGCACACTCAGTGGGCTGTTTCTCCTTATTT
CATCTGTTCTATGCTTCCTCGTGCCAATTATAGTTTGACAGGGCCTTATTACTTGGCTTTTTCCAAATGCTTCTATTTATAGAAATCCCAAAGA
CCTCCACTTGCTTAAGTATAOThTCACTTACATTTTTGTGGTTTTGAGAAAGTACAGCAGTAGACTGGQQCGTCACCTCCAGGCCGTTTCTCATAC
TACAGGATATTTACTATTACTCCCAGGATTCAGCAGAAGATTGCGTTAGCTCTCAAATGTGTGTTCCTGCTTTTCTAATGGATATTTTAAATTCATT
CAACAAGCACCTAGTAAGTGCCTGCTGTATCCCTACATTACACAGTTCAGCCTTTATCAAGCTTAGTGAGCAGTGAGCACTGAAACATTATTTTTTA
ATGTTTAAAAAGTTTCTAATATTAAAGTCAGAATATTAATACAATTAATATTAATATTAACTACAGAAAAGACAAACAGTAGAGAACAGCAAAAAAA
TAAAAAGGATCTCCTTTTTTCCCAGCCCAATTCTCCTCTCTAAAAGTGTCCACAAGAAGGGGTGTTTATTCTTCCAACACATTTCACTTTCTGTA
AATATACATAAACTTAAAAAGAAAACCTCATGGAGTCATCTTGCACACACTTTTCATGCAGTGCTCTTTGTAGCTAAACAGTGAAGATTTACCTCGT
TCTGCTCAGAGGCCTTGCTGTGGAGCTCCACTGCCATGTACCCAGTAGGGTTTGACATTTCATTAGCCATGCAACATGGATATGTATTGGGCAGCAG
ACTGTGTTTCGTGAACTGCAGTGATGTATACATCTTATAGATGCAAAGTATTTTGGGGTATATTATCCTAAGGGAAGATAAAGATGATATTAAGAAC
TGCTGTTTCACGGGCCCTTACCTGTGACCCTCTTTGCTGAAGAATATTTAACCCCACACAGCACTTCAAAGAAGCTGTCTTGGAAGTCTGTCTCAG
GAGCACCCTGTCTTCTTAATTCTCCAAGCGGATGCTCCATTTCAATTGCTTTGTGACTTCTTCTTCTTGTTTTTTAAATATTATGCTGCTTTAAC
AGTGGAGCTGAATTTTCTGGAAAATGCTTCTTGGCTGGGGCCACTACCTCCTTTCCTATTTACATCTATGTGTATGTTGACTTTTTAAAATTCTG
AGTGATCCAGGGTATGACCTAGGGAATGAACTAGCTATGGAAATAACTCAGGGTTAGGAATCCTAGCACTTGTCTCAGGACTCTGAAAAGGAACGGC
TTCCTCATTCCTTGTCTTGATAAAGTGGAATTGGCAAACTAGAATTTAGTTTGTACTCAGTGGACAGTGCTGTTGAAGATTTGAGGACTTGTTAAG
AGCACTGGGTCATATGGAAAAAATGTATGTGTCTCCCCAGGTGCATTTTCTTGGTTTATGTCTTGTTCTTGAGATTTTGTATATTTAGGAAAACCTC
AAGCAGTAATTAATATCTCCTGGAACACTATAGAGAACCAAGTGACCGACTCATTTACAACTGAAACCTAGGAAGCCCCTGAGTCCTGAGCGAAAAC
AGGAGAGTTAGTCGCCCTACAGAAAACCCAGCTAGACTATTGGGTATGAACTAAAAGAGACTGTGCCATGGTGAGAAAAATGTAAAATCCTACAGT
GGAATGAGCAGCCCTTACAGTGTTGTTACCACCAAGGGCAGGTAGGTATTAGTGTTTGAAAAAGCTGGTCTTTGAGCGAGGGCATAAATACAGCTAG
CCCCAGGGGTGGAACAACTGTGGGAGTCTTGGGTACTCGCACCTCTTGGCTTTGTTGATGCTCCGCCAGGAAGGCCACTTGTGTGTGTCGTGTCAGTT
ACTTTTTTAGTAACAATTCAGATCCAGTGTAAACTTCCGTTCATTGCTCTCCAGTCACATGCCCCCACTTCCCCACAGGTGAAGTTTTTCTGAAGT
GTTGGGATTGGTTAAGGTCTTTATTTGTATTACGTATCTCCCCAAGTCCTCTGTGGCCAGCTGCATCTGTCTGAATGGTGCGTGAAGGCTCTCAGAC
CTTACACACCATTTTGTAAGTTATGTTTTACATGCCCCGTTTTTGAGACTGATCTCGATGCAGGTGGATCTCCTIGAGATCCTGATAGCCTGTTTCA
GGAATGAAGTAAAGGTCAGTTTTTTTTGTATTGATTTTCACAGCTTTGAGGAACATOCATAAGAAATGTAGCTGAAGTAGAGGGGACGTGAGAGAAG
GGCCAGGCCGGCAGGCCAACCCTCCTCCAATGGAAATTCCCGTGTTGCTTCAAACTGAGACAGATGGGACTTAACAGGCAATGGGGTCCACTTCCCC
CTCTTCAGCATCCCCCGTACC
HUMAN SEQUENCE-CODING
ATGAACCTGGAGGGCGGCGGCCGAGGCGGAGAGTTCGGCATGAGCGCGGTGAGCTGCGGCAACGGGAAGCTCCGCCAGTGGCTGATCGACCAGATCG
ACAGCGGCAAGTACCCCGGGCTGGTGTGGGAGAACGAGGAGAAGAGCATCTTCCGCATCCCCTGGAAGCACGCGGGCAAGCAGGACTACAACCGCGA
GGAGGACGCCGCGCTCTTCAAGGCTTGGGCACTGTTTAAAGGAAAGTTCCGAGAAGGCATCGACAAGCCGGACCCTCCCACCTGGAAGACGCGCCTG
CGGTGCGCTTTGAACAAGAGCAATGACTTTGAGGACTGGTTGAGCGGAGCCAGCIGGACATCTCAGACCCGTACAAAGTGTACAGGATTGTICCTG
AGCCCAGCAGGTTCACAACTACATGATGCCACCCCTCGACCGAAGCTGGAGGGACTACGTCCCGGATCAGCCACACCCGGAAATCCCGTACCAATGT
CCCATGACGTTTGGACCCCGCGGCCACCACTGGCAAGGCCCAGCTTGTGAAAATGGTTGCCAGGTGACAGGAACCTTTTATGCTTGTGCCCCACCTG
AGTCCCAGCTCCCGGAGTCCCCACAGAGCCAAGCATAAGGTCTGCCGAAGCCTTGGCGTTCTCAGACTGCCGGCTGCACATCTGCCTGTACTACCG
GGAAATCCTCGTGAAGOAGCTGACCACGTCCAGCCCCGAGGGCTGCCGGATCTCCCATGGACATACGTATGACGCCAGCAACCTGGACCAGGTCCTG
TTCCCCTACCCAGAGGACAATGGCCACAGGAAAAACATTGAGAACCTGCTGAGCCACCTGGAGAGGGGCGTGGTCCTCTGGATGGCCCCCGACGGGC
TCTATGCGAAAAGACTGTGCCAGAGCACGATCTACTGGGACGGSCCCCTGGCGCTGTGCAACGACCGGCCCAACAAACTGGAGAGAGACCAGACCTG
CAAGCTCTTTGACACACAGCAGTTCTTGTCAGAGCTGCAAGCGTTTGCTCACCACGGCCGCTCCCTGCCAAGATTCCAGGTGACTCTATGCTTTGGA
GAGGAGTTTCCAGACCCTCAGAGGCAAAGAAAGCTCATCACAGCTCACGTAGAACCTCTGCTAGCCAGACAACTATATTATTTTGCTCAACAAAACA
GTGGACATTTCCTGAGGGGCTACGATTTACCAGAACACATCAGCAATCCAGAAGATTACCACAGATCTATCCGCCATTCCTCTATTCAAGAATGA

TABLE X

MOUSE NOMENCLATURE
ICSGNM Notch1
Celera mCGI8747
HUMAN NOMENCLATURE
HGNC NOTCH1
Celera hCG178O817
MOUSE SEQUENCE-GENOMIC
GTCTGGAGGTTTTATTTGTCCCTGAGGGGGTGCCATTTTGCTTTGGGGGTATGATATGAAGAACCTGTGTCTGAGAGCGGAGTTGTAGAGAGTGGGC
AGCAGCCAGCATCCTGGCATAGTCCCCTTGCTACAGGGCAGGAACTGAAGAGAGAGAAAGCACACACAGTCTCCAGTCTGCTCTGCTTCCCGCGTCC
ACTGTGCTATTGATGTTTGAAAGTGGAAGGAACCGTGAACTCTTTTATTACCCAGATGATGCATATTCATGACAGAAGCTGGGAGAGAAAAGCAGAC
CAGCCTCCTAAATGAGAGTTTCACAGAGTCTATGTCGACATCCCCCCACAGGGCTGGGGGGAGGGGGCTCTGCTGCGCCTGTAGGCTCACTTCGAGT
GTAGACCCAGATTTGAAATCTGCCTGGTTTGAAATTCAGGCTGGTCTTGTCTCTAGCCTGGCCCCAATTAACAGTCATTATTATTTCTTTTITATTG
AGGTGAOGTTCACTTAGTATGTGAAAGTAACCCCTTTAAAGTGAGCGCCTCCATGGCATGCTCCAGAGCTTACGTCATCCCAGAAACCTCTGCCCAG
CAAGCAGCCACTGCACCTCCATTCTAGAGCCTGATTCCAGTTGCACCCACTGCCCAAGGTCGGGTAGCCTGTGGCCATGAGAGATGAGGGCGGGGAG
CTGACTGCCCTACATGTCTGTCAACAGCTGCAGCGGAGCCTTTGTGGGCCAACGATGCCAGGACTCCAATCCTTGCCTCAGCACACCGTGTAAGAAT
GCTGGAACGTGCCACGTTGTGGACCATGGTOGCACTGTGGATTATGCCTGCAGCTGTCCCCTGGGTTTCTCTGGGCCCCTCTGCCTGACACCTCTGG
ACAACGCCTGCCTGGCCAACCCCTGCCGCAATGGGGGCACCTGTGACCTGCTCACTCTCACAGAGTACAAGTGCCGCTGCCCACCAGGGTGGTCAGG
TAAGGGCTATAOGAOGTTGTGAACTTCACGGTTGGGACCCTTAGGCCTGGGCCCAAGGACCCTATCTTTGTTGACTTGACATACTCTGGTACACTAA
ACTTTATCCTCTCCTGGATCAGAGTTAAGCTGGGATCTACTATCCAGAAAAATCTTGGGGATTCCTTGGGAACTCTGGTCCTTTTCAACCCCAAACT
ATCTGATTTTTGGCTGCCCTGTTCCAGGACCCCAACCTCTCACTGTTTCTGTGCGTAAAGAATGAGTCCTCATGTAGCCCGGGTCTTACCATCTCCT
TGTCAGGGTGCCTCAGGTTTCCATTGCTTCTCTATGTGGGGTCCAGGAGACAGCTAGGTACAAATTTTAGACTGCCATTCCTACTTTCTGTCCTTGA
GCAGTCACTTAGTCTCTTATGACTTACAGTCCTCTGTATGCAGAAAGATGGCCACATTCTGCAACAGATACTGAGCATCATGAAGATAACACTTTAG
ATCTATCCCTGGTTGGTACCTACATGATGTCCAAATCTGCCCTGGGTTCACAGGGAGGCCCTGGCAACAGCTGACCCTCTTCTCTGCCTCCCAGGAA
AATCATGTCAGCAOGCTGACCCCTGTGCCTCCAACCCCTGTGCCAATGGTGGCCAGTGCCTGCCCTTTGAGTCTTCATACATCTGTCGCTGCCCGCC
TGGCTTCCATGGCCCCACCTGCAGGCAAGATGTTAATGAGTGCAGCCAGAACCCTGGGCTGTGCCGCCATGGAGGCACCTGCCACAATGAGATCGGC
TCCTATCGCTGTGCCTGCCGTGCCACCCATACTGGTCCCCACTGTGAACTGCCCTATGTGCCCTGCAGCCCCTCACCCTGCCAGAATGGAGGCACCT
GCCGTCCTACAGGOGACACCACCCACGAGTGTGCCTGCTTGCCAGGTAGGTTCCATCTCAGTATGCTGGCATCTAGCCTGGTTGTCCATGTCCATGC
GTGTTCCACTGTCAAGCCCGAGGGATGCAGAAAGAGTTAATACCCTTTGAAGGGAGCCCAAAAAGAAOTTTAGAGAAGGGCAGCAGCCATGGAGCCT
TAGGTGTGGTATAAGAGCAGGTCAGGATAGGGATACCAGAAGTGAGGGGACACTCTCAGTGACACCACACACGTCTATTTCTGTCTGGGCCATCTAC
TGAGATCCAGTTAOTTTGGTGCATAACCTCCAAGCCTTCTTTCTGCTGATAAGAGTGCCTTCCCTTGGTATTTTCTTCATGCTGOGCCTCCCCTGAC
CCATAGCCAACTAATACCCACCATGTCAATACCCTGAGCCTCTTAGGGCAGGGAGCCAGCCTGCCTTTACAGATGCTCATGAACTGTTTGGAATCCA
CATGTGACTCTCTOTATGTTACGOGAGCACCCTGTCATGGGCAGTCCCTGGGGTTTCCCCTTTCTCTGGGGATGGTAATTGAATAACAGGGTCTTGA
GGGCCTTAGGGGCCTCACGTTTTTCATTACTTCCTCTGCAGTGTTCTTCCCACTGTGCTCAATGTCTCTGCAGGGGTOGGGCCTGCAGGCACAGCCC
TAGTCCTTTCTGTTGCTGCTCTAACGATGGTAACTCGGTGATCTAAGGCAGCTTGGAGCTGCCGCCAACCCACTTTTGGCAAGGGTGGGGTTGGTCA
ACATACCAGGCCGTTGAGAGGAAGGAOGAGTGGGCTCAGGAAATAGGAATAGATTTGGTTGCTGGGGCTGACCTCGGACCACTCCCCTTGCAACCCC
GGGGCCACCCTACACACTGAAAAAGGGAAGTTCCCTTCTGCCCCAAGCTTGCAGAGCCAGCCTCAAGACTGCGTOCAGACACTGGCTTATACATTTC
CTGAAAGTCATTGGGCAAGCTCGGAGGTAGCCGACAGCTGCCTACACGCTTCCCAGCCTCCCTGGGGGGGCTGGOACAGGATCCTCACGCTCAGGAG
GGCCCCAGAGCTGAGTTCAGCTCTGTCTTGGTGGCCACCTAAGACCTCAGCTGTTAGAACCTGTGTTGAGAGGCTAGACTGGGTCTGTCATGCAGAC
TGAGAGAAAGCCTTGTTCAAGGAGCTTGTGAATGGAGCTGCCACCAATCCACCTTAGGCAGGGTGGGGTGGGTCAACTCAGAGTGCTGGAAGATGGG
CAGGAGGGAAGCAAGGACTGGGTGGGGTCCCTTTACCTTAGCCGGTGGGTCATCCCTGAAGACGGAACACACACTACCCTGTGCTGTGTGGAGACAT
GCTGTTTCAGAGTAGGGAGAAGTGAAAATTOTCCACCCAAATTGCTGTTGAAGGGAGTCCGGTCTGGGCACAGTTATACCCACCATTTGGGCTCCCA
GGTAGCTATTTACCAGGTTTCTGAAGAATGTATGAACTAGGGTGGACATAAAGAACTAAGATGGOAACAGAACTTAAGCCTGAGAGCTGACTGGGAA
TTAAAGCGACTCTOGCTCCTGTATATCTATCCTCTCACTCTCTCACAAGCCCGGTTTCCCTGGAGGAGGCTGTATAGSCAGAGAAGGCTTTGGCCAG
GCTCCACATAGGACCTATGATCCCTTCTCCTCGCCCCCAGTGAAATCCTTCTACACCTGCTCAGGGCCCCTCCAGGCTCGCTTCTTCTTGGAAGCGT
GCCCTGTGCTAGAATAAATCTCTGCACAGGCAGGGAGACAAGCTATTTGGTCTTGCTGACCCCTGGCCGGGCACTAGGCTCCAAGTTGGTTCCTCCC
TCACCCAGATAAGCCTTTTCCAAGCCTCCAOCTTCTTGCTGTGGTGCAAGATGGTGGGGTGGAGGGGCATCTTTGAGGAGGAGGCAGACAGGTGGGT
TCTCCAAGGGCAGOAGGGGTTCAAAGGCATCCCCAGAGATGGTGTTAATTAGTAACCOGCTGCAAGTTTGGCTGTTGCCAGACTCGCCTTTCTGGGT
TGATCATTGACCCOGCAGGAGTTGAGGAGATTAATAGCTAACTAGCTGCTGAGCAGGCCCTCAGGAAGCCACACrGCAGGCCACACCTTGGGGCCCA
GCCCCAGGCTAGATGGGCTTGTAGAAGAGAGATGGTTGCCCTGGCTGTGCCCTGAGGAAATCACAGGTTCTGACTGAGCGGGCCTGCCTGGATCCCA
CCACTCTGGTTTCTAACTTAGTGCTCCCAAGCCCCACTTTTAGCTCCAGCAGTTTTAGGTATTCGTGGCTGCCCCACTGATTCAGTGTGGACAACCA
TAGAGTGCAATCCTCTGAAACTCTGTTAGTGGTTTTGTGGCAGAACCTCCAAGOACACTATGTTCCTAGATCCCTGTCTGTTGAGTCAGCTCTTTAC
ATAGCGACTTTCTAGAGAAGAGCTGTCACCTGTGGACACGGGAGTAGOGATGCCACGGAAGGACAGACAGATGCCTTGTCCAGAQCAGAACATTGCA
GGTCCACCCAGAATCTGAAGATACCGGAGCCCACCCGAGGCTCCTCAGCAGACTTCTGAGGTCAGGCTAACCTGCTGGCTGGCCCCTGTCCCACAGG
TTTTGCTGGACAGAACTGTGAAGAAAATGTGGATGACTGTCCAGGAAACAACTGCAAGAATGGGGGTGCCTGTGTGGACGGCGTGAATACCTACAAT
TGCCGCTGCCCACCGGAGTGGACGGGTATGTATATGGGACTTGTAGGCAGCAGCCTTCCACTAACAAAACTGCACACCGCGGAGTGGTAGGAAGGTC
TGAACTCTGTTCCACGCTATGAAAGCCCACACTGCAAGACCCAGGGTGGCTGGTAGCATGGGAGCAGCAGCTTCTGTOCTTCCAGTTGGAGTCAGCA
CAGCTCAGCATCAOATGGTTCTTGTGAGCTATTAGCAGCTAACATCCAGAGTCAGGCCCCCAGCACTGAGCCATACAGGAGCTAGCATTCTGGGCAA
GCTACCAGGGATGAATACAAGGTTGTCCTCAGCCCCACAGCCCAGAGCACGGAGCTCATCTTGTAGCTGAGGTTGATGTTCCTGTGACCCCTCATAG
GTCAGTACTGTACAGAGGATGTGGACGAATGTCAGCTCATGCCCAATGCCTGCCAGAATGGCGGAACCTGCCACAACACACACGGCGGCTACAACTG
TGTGTGTGTCAATGGGTGGACTGGCGAGGACTGCAGTGAGAACATTGATGACTGTGCCAGTGCCGCCTGTTTCCAGGGTGCCACTTGCCACGACCGT
GTGGCTTCCTTCTACTGTGAATGTCCGCATGGGCGCACAGGTAAGTCGGTGCAGGAATGTCCTGGGAGAAGGGAAACTTGGTTGGCTCACCACCCCT
GGTGGAAACACCTGGGGAGCCATGTATGAGATATATATACAGATATATGTGTGTATATACATATATATATATATATATATATATATATATATATATA
TATATCAGATATAGATACAGCTCAGATGTAGATACAGGGCTGGAAATGCAGCACAGTTGGTAGAGTGGCTGCCTAGCATGCACAGGATACTGGACTC
AATCCCTAGGACCACATAAACTGTGGGTAGTGGCACGTGCCTGTCAAGCCAACTCCCAGGGAAGAGTGAATCCAAAGGTTAAGGCCATCTTTAGCTA
TATAGCAAACTTGAGTTCAGCCTGGAATATGTGAGACCCTGTTTTAGGAAAGGGATGGGGAGAGAGAAAGCTCTGTCTCTTTGGCACCTCCAGCAAT
GACCCAGTAAGCCTTGCTGGACAGAGGCATCCTGGTGATCTGAACTTTGTGGGATGCTTGAGTGAGTCAGAGAGATTCATTCCAGGGTGTTGGGTGA
GGATGGCCCTGCTGCAACAACCTGTTAATCCCAGGATAGGGAAAAGCAGCACCCACTCACCGCAGCTGCCTTGTCTAGGTCTGCTGTGCCACCTCAA
CGATGCGTGCATCAGCAACCCCTGCAACGAGGGCTCCAACTGTGACACCAACCCTGTCAACGGCAAAGCCATCTGCACCTGCCCCTCGGGGTACACA
GGGCCAGCCTGCAGCCAGGACGTGGATGAGTGTGCTCTGGGTAGGTACAGATGCAGTGGCACAGGCAGCAGGAACGGGCTGGCTGTCACATATATGG
GATGTGATCTGTCCTGGAGAACCACCATTTTGATGGCCTGGGCTGGGGCATTGCGGGTGGTCQGGAGGCTGCAAGGCCAGATGAGCCCTCATCCGTG
CCTCTTTCACCAGGTGCCAACCCTTGTGAGCACGCAGGCAAATGCCTCAACACACTGGGTTCTTTTGAGTGCCAGTGTCTACAGGGCTACACGGGAC
CCCGCTGTGAGATTGATGTTAATGAGTGCATCTCCAACCCATGTCAGAATGATGCCACTTGCCTGGACCAGATTGGGGAGTTCCAATGCATATGTAT
GCCAGGTATGTCTTATGCCATTTCCCACCATGGGGGCTGCTAAGAGCAGGCAGCAGGGGAATGAGTCAAGGCTGCATAGCTTCAAGTAACATTTCTG
TCATGGTTGGTAGCATCTGGGAAGCCGAGGCCAGGATAACAGGACAAAGTGGAGAATGTACCCAGAGTTGGGTGCATAGACTCAGTAGATATAGACA
TGAACCACAGTGGGCACACTAGGCCCGTATGAGGGAGGGTTATTTCTCTCCCAAGTCTTCAGCTGGGGCTCTAGAAGGACCTACCGAAGCCGTCCTC
GGTCTCACCATCTCCCGTCCACAGGTTATGAAGGTGTATACTGTGAAATCAACACGGATGAGTGCGCCAGCAGCCCCTGTCTGCACAATGGCCACTG
CATGGACAAGATCAATGAGTTCCAATGTCAGTGCCCCAAAGGTGAGGCCACCTACTTACCACAGGACCCGTATCCTCATGATCTCTTCCCAGACTCC
TTTGAGGGAGGACTTGAGTTGACTAGGGCGTTTTCAGGCTTTCCTCAAAAGGTAAAGGCCCACGTGAGCAGTGGACGCTGTAGGGGACCTGGCACCG
GAAACCCTTTACTAAAGGAGGGAAGGATGGGCAGGAGCTGCAGCGCTCCTAAGATAGGAGAGTCCACCTGCTTGGCTCTGTCCTCTTCCTGATGGTC
ATGGGAGTAAGGACATAGTGTGGGGATCTGACCTCTATGCCTGGGAAGGACTTGGAGGAACTGAAGAGGGGACCAAAGTCCTGGGAATCTGTCTCTT
GCCTACTCCAGGGCTGGACCAGGCCTTGGGCCTTCTGAGATGTAAATAAGCCATCCTAGACTTGGTCCAGCTAGGGGAGTCCCATTCACAGCCTAGT
GCAGCTGTAACCATAGCAACCAGAGTCTATTTAGGAAAAAGGCCCCTCTGTTGGGCCAAGGAGGCAGCCACTGAGCAGTGCTAAACAAGGAGCTGAT
TAGAGGGGCAGGGTGCAAGGGGCAGGATATAAGGGCTGGTTGAAGCCTGGAGGTTAGTGGCCCCTGTTATCAAAGTACCCTCTAGGGAGCCCATGTG
AGCAGACATTCAACTCTAAGGTTTGTGGGTAGCAACCAAGGGGTTAGGATACTGTCAGATATAACCATCTCGTCTTCCCTGGCCACAGGCTTCAACG
GGCACCTGTGCCAGTATGATGTGGATGAGTGTGCCAGCACACCATGCAAGAACGGTGCCAAGTGCCTGGATGGGCCCAACACCTATACCTGCGTGTG
TACAGAAGGTACAAGAGGCAGTGCAAGATCTGGGGGAGGGAACTCTTGAGGCTAGATAGTGCTGAGAAGGGGACAGGAAACTGGGGAGGCCTTAGTT
TGGGGCCATATAATCTGAGAGTCAGAAACTTAACTCCGCTTGGACTCCAGGCAaGAGTGATGAGAACATCCCTGGGTATGTGATACCCCACATCCAT
CGATGACCAGGCAGTGATCCTGGGCCTGCTCGGTGGTTTGCCAAGGTGTGGCCAACTAAGTGTTGAGGGAGTAACTGGCCCTGAAGCTCACTAATGT
CCACCTTTCCTGCCATCTAGGTTACACAGGGACCCACTGCGAAGTGGACATTGACGAGTGTGACCCTGACCCCTGCCACTATGGTTCCTGTAAGGAT
GGTGTGGCCACCTTTACCTGCCTGTGCCAGCCAGGCTACACAGGCCATCACTGTGAGACCAACATCAATGAGTGCCACAGCCAACCGTGCCGCCATG
GGGGCACCTGCCAGGACCGTGACAACTCCTACCTCTGCCTATGCCTCAAGGGAACCACACGTGACTGGCCGGAGCTGGCAGTGGGTGGAAAAGGGTG
AGGCCAGGCTTGGAAGCTGACCACATGTCATGCTGCCCCTAGGGCCCAACTGTGAGATCAACCTGGATGACTGCGCCAGCAACCCCTGTGACTCTGG
CACCTGTCTGGACAAGATTGATGGCTACGAATGTGCCTGTGAACCAGGCTACACAGGTGAGTGGCTGAAGGCAGAGGTCAGGGTCATCCCTAGAGTC
TGGGAGGGACTGTGGGGCTAGCCAGGGTCCAAGGGCTGGATGTCACAAGCTGGGTGTCAACTACCCAGATCTTGATCTGCCAACCAGTAGAGGTATC
TATCATAAAGACTGGGTGGCCTCCATATGCCTCCTTTCCCCTGTCAGTGTCAGCCCTGGCCATGCCAGACTTAAAGCCCCAGTAGCCTGAAGTGGGG
TGGCCAGAGCTCCGAGGCCAGGCTTTTGGATATGTGTTTAGATCTGATAAAATCCTTAGTTTCTCTGAGCACCGAGGACATTGTCCACCAGAGGATA
GAAGAGGATGGGTGGTCTCAGGCTGAGGTAAAGCTGGGCTGCAGAGTGACTCAGTTGGTAAGGTGTTGACCTAGCATGTGTAAACCCTGGGTTCAGT
CCTCCTGCCATGTTAACCAGATGCAGTGGCATGCATACGTTGTAACCCTAGCACTCTGACATTGGAAGCGAGAGGATCAGAAGTTTAGGTCATTCTC
AGCTACATGTGAGTTTGTGGCCAGACTGGATCACATGAGGCTCAGAAGCTACTGGCACTACGGTCATGTAACTCATCGATACATAAGGCACTTTACT
ATCGCTCAAAGTTTCATAGATTAGCACAAGGGGAAACTTAGACCCAGCTAGGTCTAGCTCTAGGAGGGGTAGGCTCTGGGTGGCAGGCCTGGAATCT
GAGCCCCTTCCCACCCACAGGAAGCATGTGTAACGTCAACATTGACGAATGTGCGGGCAGCCCCTGCCACAACGGGGGCACTTGTGAGGATGGCATC
GCGGGCTTCACTTGCCGCTGCCCCGAGGGCTACCATGACCCCACGTGCCTGTCCGAGGTCAACGAGTGCAACAGTAACCCCTGCATCCACGGAGCTT
GCCGGGATGGCCTCAATGGGTATGGCAGACTGACCTGGGAGGGACTAGCTGTAGCCTTGGGCAGACACAGGCTGGGTGAATGGGTGCTCCTGTGAGA
CCAGTGATCTGAGGTCAGCCCCTAGTGTCTTCCCAGCATCCCTGGGCTGTATCCTCATTGGCTAGCATCAGCTCAGGATATAGACTCTAAGCACACT
GTGGCTGCTGAAACAGTTGTCTGATACTGTGGTGACAGCAGCCCTTTGTCAGTGTGTGGGGGACGAGGAGATTACTAAGGTCTCCGTTAAAACTATT
AAAGTTTTATACAGAGGGTCAGCATTGTCAAGGTGGCATTGTTCCAGTCTGGAACCCTTGCACGGTCTTGGGCCAGGTGGGTACCTCCAGCCCAGTG
GATGATGTGGCCTTGTCCTCAGACTGGAACTGTAGTTTAGCAGGAGAGCTCTGAGCCCAGGCTACAGCTCAGGCCTCTCCGTAGGTACAAGTGTGAC
TGTGCCCCTGGGTGGAGTGGAACAAACTGTGACATCAACAACAACGAGTGTGACTCCAACCCTTGTGTCAACGGTGGCACCTGCAAGGACATGACCA
GTGGTTACGTATGCACCTGCCGAGAAGGCTTCAGTGGTAAGTGTGGCGGGTGACACAGCAGAATGGGTCCTCCCTTTGGTCTCTTTCTCGGTCCACC
TGATGAGCTAGCATTTCTCAGGCAGAGATCCTAGTTCTCTTGAGGAAAGGCATAGGGCAGAGGOTCTGGCCCACCTCCTCTTGCTCAACTTCCTTAC
AGGCCCTAATTGCCAGACCAACATCAACGAATGTGCCTCCAACCCCTGCCTGAACCAGGGGACCTSCATTGATGATGTCGCTGGATACAAGTGCAAC
TGTCCTCTGCCATATACAGGTACGAACAGCCAGGGACTGGAATTGGCTCTGGTGGTAGCTAGTGGAGCTAAGAGTATATGGGCTTACCAGGCACTGA
AGAAGGTTCTGTTCCTCGCtGAGGGTCTTAGGTCCTCAGACTGGAACTGTAGTTAAGCAGGAAGGCTCTGAGcCCAGGCTACAGCCCAGGCCTCCTG
GGCCTGGGGATAGTCACCAGATGGGCAAACACAGAGGATGCCTAGGAGGAAGCTCCAGGCCGTGTGTCCGGGTGTTTATCTGGTATGTCTGGAGTGA
GGTGAGCACTGGACTCCTCCAGAGAAAGTCTGGGAGGCTGACTGGTAGGTACTGCATGGATCAGGGTCCACTTTGCCTGTGCCTCTGGGGGCCCTGG
CTGCCCTCCTCTGCTGGCTGGGCCTGGGATTCCTTTGATGGCAGCTTCTTTGTGTGAGGAGACAAGGAGCCCCCTGTTAGATGAACAGAGGCAGCTC
CTGCCATGGGCACTGCCTGAGGAATGGCGAGAAATAGACCCCCTGCAGGAGAGATGTGCATTGAACATGAAAGcCGTGACCACATGGCCCCCAACCC
TGCAGCTCCCAATACTOCCACAGAGTACTGGTGGCTTCATGCTGCTGCTGCTAGGTTCTGGACATAGCAAGTGGGTCTGTGAAGGTGGAGGCCCTTC
CAGTGTTCCTATGATGCCTGGCAGGATGCTGTTTTCTTTGACAAAGTTAAAGGGCCTGGATTTTGTCTCCTGTGTTGGTCAGTGAGGAAACTGTCAG
AATTGTCGGAAACTCTGTCAAAGTTCCTGTCTGCCCAGGTCACCGTGGAAACCGATAGAGAGAGAGGCCCTGGAGAGTCTCACCAGTGGGCTGGGGA
CATGGTGGGAGACAAGGAAGATAAGGTTTCCTTGCTTAGCTCCTGGAGCCAACCATGGCCATAAACAGGAAAGCTAACTGGGATTGCAAGGAGAGTC
ACCTGGGTTACTCGTGGCTCCCCCAAGCGGTTATCAGATTTCACTGCCCTAGTGTGAGGCTGCACCCCATGTTCCCCAAGTAGGATATGCTTGGGGT
AGCCACAGTAGCTCTGTCTCAGGAGTGAGCCTGCTGTGACCTAAAGCTTCAGAATCAGCTCAAGGATTTTCCTGGAATGGGGAGCAGGCCAGGTTCC
TCCAGGCTCAACCACCTGTTATAGTAGGGTCTTGTTTTGAGGTTTTCTGGTTGGGCAAGGCCCAGGCTTCATCGTGGAGCATTTGGGAGATGAGAGA
TGGCTTCCGGGACTACATAGATGGCTCATGGAGAAAGAGTGCTTGTGGAAACATGAAGCCTGTTTCATGAAAAGAGGGTACCTCTAAATGCCCATAA
CCCCAACATTTGGGAGCAGAGACATCTTGAGAGCTCTCTGGCTGGCCAGCCTAGCTAAGATGGCAAAGCATCTGATTCAGTGAGACTCTGTCTCAAA
GGAATAAGATGCGGGGTTGGTAGAGGACACCTGATATCCGTCTCTCCTTCTGCATACACTCTTACACACCTGCATGCAATATACTATGCTCGTTAAA
AAAAAAAAAAAGTGACTTAAGAGAGAAACTTGATTCTGCTTTATCTCTAACCTCCTGTCAGTAATCGTCATTCCTCCCGGGATCAGAGTCTGGCTGG
CCAATCATACAGCATCTCCTATACCTACCCCCCGCCCCCCTTTATGGAAGACCCTATGGGCCAGGCTCACTGGGTTCTATCTTCTACAAATCTTTCT
TTCTAGGAGCCACGTGTGAGGTGGTGTTGGCCCCATGTGCTACCAGCCCCCTGCAAAAACAGCGGGGTATGCAAGGAGTCTGAAGACTATGAGAGTT
TTCCTGTGTCTGTCCCACAGGCTGGCAAGGTGAGGCTTGCCTGGTCCCACCAGGCTGGGACAAGGATGGTGGATGGGAAGTCAGGGGCCCTGGCCAG
CCTGTGGGGCAGTCTCGAGAAAGGAGCCCATCAATGCTGAGCTACCTGCTCTGGGAAGCTCTGGGAAGCTCTGGGAAGCTCTGGGAGCAGGAGATGG
TCCTTCCCATTCCAACTCCATATCCTTCTGTTGCCCAGAGGTCAGGGGAGAAGCTCTGGAGATAAACAGACCAGCTAGGACAGAGGCTCAGAGGGTG
GGGGCAGGTAACCAAATGAGTTTTTTGTCTTCACAGGTCAAACCTGCGAGGTTGACATCAATGAGTGTGTGAAAAGCCCATGTCGCCATGGGCCTC
CTGCCAGAACACCAATGGCAGCTACCGCTGCCTCTGCCAGGCCGGCTATACAGGTCGCAACTGTGAGAGTGACARCGAATGACTGCCGCCCCAGTAA
GTGGTACCCAGCTCTGTCCATCCTATGTTTGGGTGCCTCTGTTCCTAAGCACACATGACAGGGAGCTGCTGAGAGTCTGAGGTGGAAAATAGCCCTG
GTGCCCGGCAGTCATGAAGOACTGTGGATAGCTGTGCCCTCAGCTTAGTCCACTTGCCTGTGCCTTAAAAAGAGACAGGCCAGAGGCTAGACAGGCC
AGGAGGAGGCTGTTGAGCATCAGTGTGTGCTTCTCACGGGCTTTGCTGCTAGATTTCCCAGCCCAGTTGAGTGATATGGAGCAGAGGCACCAGGCAC
TTCAAATGATGGCATGGGGCCTAGTTGTATTCAACCTTGACACTCGGCCCTTGTCAGGACCCATTGAGTTGTGTAGGGCTTTAGAGATGCCCTGCA
TCCTGAGTCAAAGCTAAAGGCAGAGAGAGATTGAGGGAGGTTCTGCCTGGCACCATCAGTGTGTGTGTAGGGGAATGGGCTTCCCTATGACTGCCTT
GGGCCTGAGGGAAAAAGTGTATGCGGAGCAGATTCCAGGCTCAGTGCTGGGATGGGCCCCTGTTGACTACTGTCATCAAGGCTTGCTGTGAGCACTT
GCCACCCCATACCACCTTCTCAGCATCAGGTCCCCGAAGCATGGCCAGTGGCCACCTAAGGCTCATCTTTCTGTCCCTCCAGACCCGTGTCACAATG
GGGGTTCCTGCACCGATGGCATCAACACAGCCTTCTGCGACTGCCTGCCCGGCTTCCAGGGTGCCTTCTGTGAGGAGGACATCAATGAATGTGCCAG
CAATCCCTGCCAAAATGGTGCCAATTGCACTGACTGTGTGGACAGCTACACATGTACCTGCCCCGTGGGCTTCAATGGCATCCACTGCGAGAACAAC
ACACCTGACTGTACTGAGAGGTGGGTCCCAGCCTCAGCTGAGACAGGGAAAGAAATGGACTGTGGAAGACACAAAGTCTTGACAGCATGGCATCTCA
AGCTGGTGTCTTGCCCAGACCCAGTGGGCATACAGCACTAATTGAACTGAATCCAGCGTTGTTCATTGTGTTGCAGTATCCCTCCTAAAGGGGCCTT
TGTGACCTCCAGTGACTTAGACAAGGAGTTTGTGCAQAGGAATTGGGGAACTAGCCTCCTATCAAACAAGAGCTTTGTAATCTGTCTTAGTTCCC
CTTTTGCCCTGGTTGCCAGGAAGCTTAGAGTTAAATCAATCTCAAGCCTGGITTGTGATGCGCTTCCTATAGTGTTCCTATTTTGCCCTTGATTTTG
TTTTTCTCAACCACCACGTGTCTATATGCTATAATCTCTGCTCGTGATTTGGGTATTGTGGCCAGGTAGGCATGGAGTTGTCTCTCCTGGCAAGG
ACCTTAGGGAACTCCTCACTCTTCCCTGCTTCTCATCTGCCTCTAGCTCCTGCTTCAATGGTGGTACCTGTGTGGATGGTATCAACTCCTTCACCTG
TCTGTGTCCACCTGGCTTCACGGGCAGCTACTGTCAGTATGATGTCAATGAGTGTGATTCACGGCCCTGTCTGCACGGTGGTACCTGCCAAGACAGC
TATGGTACTTATAAGTGTACCTGCCCACAGGGCTACACTGGTCTCAACTGCCAGGTGAGCTGAGGTTACAGGGTATGACCCAGAAGGCCTAGGGCTC
TCCAGTGCCCATTGCCCAGCCCTGGGCCACACTACCTTCAGAGGGCCAGGGACACAGAAGCAGAGAGGCTCCCTGGTGTCATCATTGGTTGATGAGC
CAGAATGAACCCACATCCTCTGGGAAAATGCCAAGTTGGGTGCTTCTGAGGGAGACAGGTGGGCATGGGTGCCAGCCAGGCTCTTCTGTGGAGTGCA
TTATGTGGCCATCTGTCTGAAGCAGCCTGGGGCAGTGCTCACTTCTGGAGGAGGATGTCCAAGGGGAGGTAGCCAGTGGGTTGTGGAGAGCCTTGAA
AGAGAGCAAGGAAGACGTGGAGCCCAGGCCTATGTTGTTCGGAGGGCGGGCAGTGTTTGTGAGTCCTGTGCCTGGGATTTCTTAGGTGATAITCTTT
CTGAGATGGTGGGGGTGGGGGTGGGGAAGTCAGAGCATAAAATGCCTTATTCCAGTGGCGCTCAGCCTGTGGGTCATGACCCCAATAGGGGTCATAT
ATCAGATATCCTGCATATTTTTGCTACATGACAGTAGCAAAATTACAGTTATGAAGAAGCAATGAAATAATTTTATGGTTGGAGGTCACCACAACAC
GAGGAACTATATTAAAAGACTGCAGCATTAGGAAGGCTGAGAACTGCTGTGCTTCTCAGAGCCAGCCAGGACCAGGGCAGGAGGGTGCCCTAGCAGT
CAGAGTTGGTCAGGGAGAAGCTAACCAGCGCCTGTCTCCTCAGAACCTTGTGCGCTGGTGCGACTCGGCTCCCTGCAAGAATGGTGGCAGGTGCTGG
CAGACCAACACGCAGTACCACTGTGAGTGCCGCAGCGGCTGGACTGGCGTCAACTGCGACGTGCTCAGTGTGTCCTGTGAGGTGGCTGCACAGAAGC
GAGGTAACCAACCGGCCGGGCCCACCTGTCCTTGCTCAGGCTCAGCTTAGGCCAGGAGTGCCCCGGTGCCTGCCAGGTCAGGTGACAATGGGCAGTA
GTCCCGAGAATCGGAGCCCAGGCCTCAGCTCCTTCCCGTCTTGTCCCCACAGGCATTGACGTCACTCTCCTGTGCCAGCATGGAGGGCTCTGTGTGG
ATGAGGGAGATAAACATTACTGCCACTGCCAGGCAGGCTACACGGACAGCTACTGTGAGGACGAGGTGGACGAGTGCTCACCTAACCCCTGCCAGAA
TGGAGCTACCTGCACTGACTATCTCGGCGGCTTTTCCTGCAAGGTGGGGCCGTCATGGGGTGGCGGGGAGGGTATCAGCGTGAGTGTTGCTGCAGG
GGAGGGCTTTTGACTGGGAGCTAGTGCATCATGGCAGACTGAGCACATACCTGTGCAGGATCCGGTCACATTACAGATGTGCTGAAGGAGGAGAACT
ATGCCAAGCAGTGGCTTAGGGCACCCCTTTCCTATATGGTTTCATGGTACCTAATACCTGCTCCAAGACCTCATCCCTGGGGGACCAGGTGTTGCCC
CACACACACACACCTTGGACACTTATTTTAACTTACTGGGGTCTACTGGGTGGAGCCCTCGCTGCAGGGTGGGACAGTGTAGTCTAACTGGGTATAT
GGCCTGCATGGCAAGTGGAGGACATGATACCACAACCCCAGCTAAGCTGTGACCTGTGCCATTTCTACAGTGTGTGGCTGGCTACCATGGGTCTAAC
TGCTCCGAGGAGATCAACGAGTGCCTGTCCCAGCCCTGCCAGAATGGGGGTACCTGCATTGATCTGACCAACTCCTACAAGTGTTCCTGCCCCCGGG
GGACACAGGGTAAGGGTAGGGACCTGGATGATGGGCAGGATCCAGCCAGAGATCACTCAGGTGATTGAGAGCTGACCACGGAATAGGAATCTCAGAA
GGGAGGTGGGCTCCAGTGGCACTGTGCTGCCTCCCCCGGGCCCCAGGTCCTAACTGATTTCTGGACTGGGCTGCACAGGACAGAGCACAGTCAGCAT
CATCCGACGGTGTCTCTGGTCACTCTGCAGGTGTACACTGTGAGATCAATGTTGATGACTGCCATCCCCCCCTTGACCCTGCCTCCCGAAGCCCCAA
GTGCTTCAACAATGGCACCTGTGTGGACCAGGTGGGTGGCTATACCTGCACCTGCCCACCAGGCTTCGTCGGGGAGCGGTGTGAGGGTGATGTCAAT
GAATGTCTCTCCAACCCCTGTGACCCACGTGGCACCCAGAACTGTGTGCAGCGTGTTAATGACTTCCACTGCGAGTGCCGGGCTGGCCACACTGGTG
CGTGCCTTGGGCAGGTGGGTCGAACGGGTGGGTGGGCAGGGTOACAGGGATGGCTCCTTACTGTCCCTTCTTCACACAGGACGCCGCTGTGAGTCA
GTCATCAATGGCTGCAGGGGCAAACCTTGCAAGAATGGGGGTGTCTGTGCCGTGGCCTCCAACACCGCCCGTGGATTCATCTGTAGGTGCCCTGCGG
TAGGTGTCCCTGGCCCTGTGCGGCTGCAAGGGTGCCAGGTCTGAGGGTGCCTGAACCCTGACCGACTCTTATGGTGGATCTGTCCTCCACCCACCCT
CACTGTGAATAGTCAGCCTGAAGACCTCTAGCAGAGGCTAGGGTCCAAGCCACATGCTTCCTCGTGTCCATTCCCCAGCTCGTTTTTTAAACCCGAG
CGGGCACGATCCATACATGCCTTGCTCCCAGTGCTCTGCAGCCTCAGGCTGAAAGATTGCCTTGTATTTGAGGTACCTTAGGCTTCACAACAAGACC
CTGTTTAAAAAGTTTATTTCCTGCTGTGCTATGTTGGGAACCTCACACATGCTGGGCAAGTGTTTTGCCACTGTGTCTTAGCCCAATTTTTTAAATA
GATGTTTATTTTAGAGCGGTTTTAGGTTTGCATGAGAACTTAGCAAGAGGGCCAGGTCCCCACCCTGCAGGCCGTTCCCTATGTTTGCTGCCTTGCG
TGGAAGGTGTTGAGCTGGCACTAATATGCAGTATTTGCTCTTGGGTGTTGACATCCAGGGATCTGTACACATACAATGTACCAGCATCAGCCTTGGG
TTTCATGGCTCTAAAAACTCCTAGGGTTCTCCCCATTCACCTATCCCTCCAGTTTGAAGCCCTTGGCAACCCTGACCATCCTATGGTGAGATGTTTC
AAGGGTCTTGTTATTGGGGCAAGGCCATAGACACCTTTCACAGGCCACAGACTGGGGCCAGGTCTTTGCTGGGGTGTGCATGGTGTCCCTGATTGAA
TGCACCATCCTCTCTCCACCACAGGGCTTCGAGGGTGCCACATGTGAGAATGATGCCCGCACTTGTGGCAGCTTACGCTGCCTCAACGGTGGTACAT
GCATCTCGGGCCCACGTAGTCCCACCTGCCTATGCCTGGGATCCTTCACCGGCCCTGAGTGCCAGTTCCCAGCCAGCAGCCCCTGTGTGGGTAGCAA
CCCCTGCTACAATCAGGGCACCTGTGAGCCCACATCCGAGAACCCTTTCTACCGCTGTCTATGCCCTGCCA.AATTCAACGGGCTACTGTGCCACATC
CTGGACTACAGCTTCACAGGTGGCGCTGGGCGCGACATTCCCCCACCGCAGATTGAGGAGGCCTGTGAGCTGCCTGAGTGCCAGGTGGATGCAGGCA
ATAAGGTCTGCAACCTGCAGTGTAATAATCACGCATGTGGCGGGATGGTGGCGACTGCTCCCTCAACTTCAATGACCCCTGGAAGAACTGCACGCA
GTCTCTACAGTGCTGGAAGTATTTTAGCGACGGCCACTGTGACAGCCAGTGCAACTCGGCCGGCTGCCTCTTTGATGGCTTCGACTGCCAGCTCACC
GAGGGACAGTGCAAGTAAGTAAGGCTGAGTTTCTTTAGAGTCCCAGGGCTCAGGATGCTACGGGAGGACCTAACCAAAcACCAGGCTCCTGAAGCCA
ATGTCTATCCCTGCCCATTGCGAGTCGCCAAGCACATTTCCCAGATCTGGCCTATTCAAGGTATGGGTTTGAGGTCCACAGGCTGGGATGGGACTGA
GTGCATCCTATACCTCCTCAGCCCCCTGTATGACCAGTACTGCAAGGACCACTTCAGTGATGGCCACTGCGACCAGGGCTGTAACAGTGCCGAATGT
GAGTGGGATGGCCTAGACTGTGCTGAGCATGTACCCGAGCGGCTGGCAGCCGGCACCCTGGTGCTGQTGGTGCTGCTTCCACCCGACCAGCTACGGA
ACAACTCCTTCCACTTTCTGCGGGAGCTCAGCCACGTGCTGCACACCAACGTGGTCTTCAAGCGTGATGCGCAAGGCCAGCAGATGATCTTCCCGTA
CTATGGCCACGAGGAAGAGCTGCGCAAGCACCCAATCAAGCGCTCTACAGTGGGTTGGGCCACCTCTTCACTGCTTCCTGGTACCAGTGGTGGGCGC
CAGCGCAGGGAGCTGGACCCCATGGACATCCGTGGGTGAGTGCTCCAGCTCCTGCTGTTGTGGGCTOTTTCCCAGTGTGTCCCTGGGTTCTCTAACC
AGCTTAACCCTGGAGGTGTAACCTCAGGGGAGGTAGTATTCACCTTATTTTATAGTCAGAAAGCACCTGGGATGAAGAGGGCATCCCTCTGATGAGG
ACTTGGCAGGCCTCAGGGTTTGCCCAGATGTGATTTTCAGGCCCCACTGTGThCCAGGTGTTGGAGACCAAGCTGATTAAAGCCTCCAGGGTGTCCC
CTGGGGCTAAGTCATCCTAGACCAATCCTGACCCTCAGTCACTACAGCCCTTATCCCTACTACAGAGGAGGCAGTGAGGGGTACAGAAGGGCACTGT
TGCTGAGCCTGGAAGGCTGAGGAGCCGAGGGAGGCACAAATAAGAGCATCTCCTGGATGCTGCACAGAGCCATCTGGGGATGGGCAGGGAAGCACCA
GCCAGGCATTTGGAGGTTCCAGGAGCCTTCGCTGTTGGGCATCTGCCTGGTGGACCTCAGTGTCTCCTGACCCCAGTGGTTCCTGGTCCTGGTTTTT
CTTGTCGTGGGACTGTGGTGAAGGTCAGAATTAATTGTTTCCTGGAGGCTGTTAGAATCCTGTTTCTGAGAGGCCAGTGACTGAGAGTTGGTGACT
TGGCTGGCATGAGAGAGCTGACTCTCTGGCATCTGAGCCTGCTGTCACCTCAAGCTACACCATAGGGAGTCACAACTGCATCCTTGCAGTTCTGTTGG
GGCCCAGGCACTGCTCTCAAGAGGGCATTGGATGCCTAAAGACTATTCTTTGAAGTAAGGAAAAAGGGGTGCTGTGCACCTAAGGGGCAAGGCTAGA
CAGTGGAATGACCCCCGCTGAGTGCTAAACACTGGGGCAGCAGGGAGAGTGTCCCATTGCGGGGCTGTCGTCTGATAAGCCCTGTGGGTCAGGGTGA
TGGGCTAGGGAGTCAGAGCTGGTGTGTAGGCAGTGTACTGGGGTGTATCTAATCTCACTGTGCCATGTGTGTTCTCAGCTCCATTGTCTACCTGGAG
ATCGACACCGGCAATGTGTGCAGTCATCCTCGCAGTGCTTCCAGAGTGCCACCGATGTGGCTGCCTTCCTAGGTGCTCTCTTGCGTCACTTGGCAGCC
TCAATATTCCTTACAAGATTGAGGCCGTGAAGAGTGAGTAATCAGGGGCTGGAGGGATGGCTCGGTGGTTAAGAGCACTGGCTGCTCTTGCAGAGGA
CAAGGGTTCAAATCCCAGCACCCACGCAGCATCTTATAACTATGTGTAACTCTAATTCCAGGGCATCCAACACTTTCTGACCTCTGCAGGCACCAGG
CATGCACAGGGTCCTGAGTACAACAGCAGGCAAAACACCCATACACATTACATATTTTTTAAATGAGTTCCCTACCCCGACCAGCCCTGCCTCTTCAG
GGATGGGTTCATTGGGTGGCGGGTCACAGGCAGGCGGTCTCTCACTCCTTGTGCCAGCTGCCATTCCCAGCTCAGGAAGTGCTTTTCTGGAATTTTT
TTCCAAGGCCTTCCCTCCCTGTGGCTAGTGGGAGCCGTAAGTAAAAGCTGTCCCCTAAGATGTATACATATACCAGAAAGTATGGAGGAGTCTTTCT
TCTCTCCTCATTCTTTACCCCTGATTTTTCTTTGTTGGATATTATTTCAAAATCATTACTAGAGTTTTTTTGTGTGTGTTATTTTTTTTAAGAGAGA
GAGAGAATTGATCGGTGTCATGTGAAGTGTTGAAGTTTGTATCTTGAAAATCCCCCTAAATCCTAACAGTCTTAACAGCTCAATGCGAGCGCAGTGATT
TGAAGTTCGCTAATCCTTCCTTCTCGAAAGGGGAGAAGTGAGCACTGTCTCCAGACAGATCAGCTGGTGCAGGAGAGAATTTAGCGATAGTTTGCAAT
TCTGATTAATCACGTAGAAAATGACCTTATTTTGGGGGGTGGGATGGAGGAGAGTGGGTGAGGAGGCACCGGCCGTGGAGCCAGTCCTCCGCCCCCC
GCCAGCCCACAGCATCACCACGCCTGACGAGGGGTGCTTGCCTGCCGCCCCTGCCCGCAGGTGAGCCGGTGGAGCCTCCGCTGCCCTCGCAGCTGCA
CCTCATGTACGTGGCAGCGGCCGCCTTCGTGCTCCTGTTCTTTGTGGGCTGTGGGGTGCTGCTGTCCCGCAAGCGCCGGCGGCAGCATGGCCAGCTC
TGGTTCCCTGAGGGTTTCAAAGTGTCAGAGGCCAGCAAGAAGAAGCGGAGAGAGCCCCTCGGCGAGGACTCAGTCGGCCTCAAGTGAGTGGACACTG
CTCCCACTGTGTGTGGGTGAGTGAGTGGCAGGGTTGGTGGGGGTGCTTAGCTCCAGAAGGCCCATGGGCCCATCCATCCCTGTCCGTTACCCTAAGC
CCTTCTGCATGTAGAGGATGCCCATATGAGCTGTGGCTTCAGGGCATCTCCTGGAGTACCTGCTCAGTCTCCCTACCCCCATACCCAGGCCCCTGAA
GAATGCCTCAGATGGTGCTCTGMGGACGACAATCAGAACGACTGGGGAQACGAAGACCTGGAGACCAAGAAGTTCCGGGTGAGTCCCGCAGGCTCC
CAAGCCCCCCCTGGGTGGCACCTCCTGCCTTGGCCCCAAGTGATGAGGCTCTGCTTACTCTGTCCACACTCAGTTTGAGGAGCCAGTAGTTCTCCCT
GACCTGAGTGATCAGACTGACCACAGGCAGTGGACCCAGCAGCACCTGGACGCTGCTGACCTGCGCATGTCTGCCATGGCCCCAACACCGCCTCAGG
GGGAGGTGGATGCTGACTGCATGGATGTCAATGTTCGAGGACCAGGTAAGGCCACTGGAGACACATGCACACATTCCCTAGCATAGACTTAAGAGGC
CAGTAGGACTTGAGGAGGGCTGGGTCACTGAAGGGCTCTGCCAGAGCAGCATCCTGCCCTGAGCTGATCGTCTGGCTCCTCCTGCCCACACAGTATA
CTTTTTCATGAAATGCTGCTTTGCAATAGTCAAAGTGTTAATTTTTTITTTTTTTTTTGGTCTCAGTTTTTAGTATGCTGGGATTATAGGTGTGTCC
CCATGCCCAGCTAAAGTGTTGACTTTTAAAATTATATGCATATAGGTGTATATAAAGATATCTTTTATTTTATGTGCATGGTTCTGCCTACATATAT
GTCTGTGTAGTGTTTAGATACCCAGTGCCTGTGGGGAGCCTGAAGAGGCCATTGGATCCCCTGAAGCTGCAGTTACAGATGGTAGTGAGCAGCTATA
TGGGTACTAGGGATTGAACTCAGGTCCTCTGGAAGAGAAGCCAGTGCTCTTAACCACTGAGCCATC1CTGCAGCCCTTATTTTATGTTTTAAAGCAT
TTCTTCATTTGAGTGTAAGGGGCACACATGTGCCATAGTACACACGTGGAGGTCAGAGGACTACTTGTATGAGTGTGGGTTCTCTCCTTCTACCATG
TTGGTCCCAGAGTGCCTTCCAGGGAGGCAGCACTGCATCCCTGGGGACTTCATCCGGGGTCCCTTAAACTGCAGGACTGCCTGCCAGGGCTATGGTT
GAATGTTGACCCTCCCTGAGGCTGACACCATCCTTCCCACACCTACCCTTGTTTCTGTCCTCTCTGCAGATGGCTTCACACCCCTCATGATTGCCTC
CTGCAGTGGAGGGGGCCTTGAGACAGGCAACAGTGAAGAAGAAGAAGATGCACCTGCTGTCATCTCTGACTTCATCTACCAGGGCGCCAGCTTGCAC
AACCAGACAGACCGCACCGGGGAGACCGCCTTGCACTTGGCTGCCCGATACTCTCGTTCAGATGCTGCAAAGCGCTTGCTGGAGGCCAGTGCAGATG
CCAACATCCAGGACAACATGGGCCGTACTCCGTTACATGCAGCAGTTTCTGCAGATGCTCAGGGTGTCTTCCAGGTACGCAGCACTGCATCCTTGGA
GCCAGTGACCTCATCAGGTCTGACTGGCATGGACCCTGCAATCTCTAATTCCAAGTGTGACTGGGCAGGGAAACTTGGCACCTCCACACTGCATTTT
GAAGGAAGTCCAGGTACAAAGGGCATCAAGTCCCACCTGTGTTCTCTGAGCAGGCTGGCTGAAGGTGGTCTTGCGGAACCTACAGGGTGAGGCAGGG
CCTCCTTACTCTCAGTAAAACCCTCCCAATCTCAAGGCTATGGCCTTGACTTGAGGTCTCCACCTCCGTGCTAAAACCTTTGCAGACTAACCCTACC
CCATTCAGCTGTGGCTGCAGGCATAGCCCATGGCGGCAGAAAGAGAGCTTTAGAGGCTGCTGGTCCTGGCCTGCAAAGCTATCTCCTGGCTTTGCCT
AGAAGCCTGGAGCCAGCCTGGGTGGATTTAAGGATTTAGCTGOCTGGGTCTGCAGGGACCAACCCTAGTAGTTGCTCCTGCATGTTGCCAGCCAGTG
ACCCCTGCTACCAGACCTGTCCTCTAGGCGGCCCTGGAGCAAACTCCAATGGGGAATAGACCAGGCATCAAAAAGCTTGTCATTTAATCATTTCTTA
ACTTGAACCACACTTTGTCGGTCTGTAGAAGAAATTAAACAAGACAGGGCAATGTACACCTTTTATCCCAGCACTCTAGAATCTGAGAGGATTGTGA
GCATGAGGCTAGCCTGGTCTTCATAGAAAGACCCTATTTCAAAATAGCAAAGAGAGGTTGAGACCAGCCACAGGTTATGTTGACTGCCTGGTGTACA
GCTCTCCACATGGCCTGCCTTGCTTGCATAGACAGATGTGTGTACGTTTTGTAGGAATCCAGGCAGTGCTGTGAATCCTTACCCAGGTCACCAACCC
ACTCCCTCCTCTCACCTTAACAGTTCCAAGTACGGCTATGGGCCATGAGCGGGATTCCTAGCCTGGTTACTCTCCCCTCTCTCCCCCATGCTATGCA
GTTTGATTTCCTCAACCTGGAGGTAGTTCTGGCAACCATAACCAAAAGATGAGGTCCCAGTAGATCCCCCACTGTCACTAGGGACTGGGTAAGAGCC
CCAGCTCTTGCACATGGAATGTAGCTCCTGCCTTCCTCAGGGGCTCAGCTGAGCCCAGTCCAGGCATTGGTTTCTGGAGATACAGGCTTGCATTTAG
ATCACCCTGCCTGAACCCATCCCTGCCTTCCAGATCCTGCTCCGGAACAGGGCCACAGATCTGGATGCCCGAATGCATGATGGCACAACTCCACTGA
TCCTGGCTGCGCGCCTGGCCGTGGAGGGCATGCTGGAGGACCTCATCAACTCACATGCTGACGTCAATGCCGTGGATGACCTAGGTAGGCCCGAGCT
ACAGCCCAGCTACCGAGGACATCAGCCAGTGCCTGGTCTGCTCAGCCCCAACCCACTCTCTCCTCTCTTTGCTTCCTTTCCAGGCAAGTCGGCTTTG
CATTGGGCGGCCGCGGTGAACAATGTGGATGCTGCTGTTGTGCTCCTGAAGAACGGAGCCAACAAGGACATGCAGAACAACAAGGTAAGCAGACGGG
CTGCTTAACCGCAGGCGCATGATGGGGACTCCGGGGCACCCAThCGTTGACCACTTAGAGGTTTAAGAGTGTCTGGGACAACAGCAGCCTAAAATAA
CCCGTGCTCTTGOTGGAGAGGAGCTGAGGCGTAAGCAGCTGTCCCATGTCACGGTTACTTGAGCCTTGCCTTCTGTCCCCTTCTGTCQCTGGTGGCT
GTGGTGACCCTAGGGGTTCAGGATTGGCCAAGGGGCAGGGATGGGGTGGCCCTGTCTCTTAAGGCCTGCAAGGCTTCTGGCTTGGTCCACCACTACC
ATGCTCCCTCCTTTGGGATAAGAAAACTGCTGAGCTGGGCTGCGTGTCTQCACCTCGCCTCCCAGTGTCTCTGTTTGTGCAGGGCTAGTTGCTTCAT
CAGGACTCTGCTTAGTTGCTCACAGGGCAGAGTTTGTGCCTCCCTTACCAGCTCCACTGCACGCTGGGAAATGGAGCAGGAGTCAGGTACCTGGAGT
TGCTGTTGGGCACAGGTTCTCCACAGTCCAGGGTGGGCCAGTGGTTATAGAGAATCCAAGCCTGATAGACATTCTGTTGTGGTGTATGATTATGTCT
GCTGGACAGAGCAGACCTGGAAGGAGCTGAGATGCCCTAGCCACTGGTGACCCTTTGGCATGGTAGTACCTTCCCCATGGCCATTCACTTGCCTTAA
AGGTCCCTGAGTCCCAGGATTGCAGTACTTGTGCCTGCATTTTGGGGAGGAAGCAAAGGGCACACAGTTACATTCTGTTTTCGGCASTCTGACTCC
ACACAGGGGAAGAAGCCCTATGTATTTATCCCACAGATCTCTGGGGTGTGAGGTGTTAAATCTAGGTTCTCTAAAAAAGAACTCGGGACCAGCCAT
GTGTGCGTGCTAGCACCGAGAGCTGAGGCAGGACGATACTGAGTTCCAAGTACGGCTGTGGGCCCTGAGTGAGATTCCTAGCAACATAGGAAGGCC
CCACTATAAATACTCTCTGAGCCTGGTGGTCTAGGATGCTTGGGTCTAGATAGATCTCTCCAGATAAGAGAAGAGAAAGAGGCAGCACCTGCTTCCC
TAGCCCCTCCCCCCAGGCCATCCATCATCAATGACTCAGCAAATTCTACACCTGCCCGCTCCCTCATGTACCTCCTGTCCGTGTCCTACAGGAGGAG
ACTCCCCTGTTCCTGGCCGCCCGTGAGGGCAGCTATGAGACTGCCAAGTGTTGCTGGACCACTTTGCCAACCGGGACATCACGGATCACATGGACC
GATTGCCGCGGOACATCGCACAGGAGCGTATGCACCACGATATCGTGCGGCTTTTGGATGAGTACAACCTGGTGCGCAGCCCACAGCTGCATGGCAC
TGCCCTGGGTGGCACACCCACTCTGTCTCCCACACTCTGCTCGCCCMTGGCTACCTGGGCAATCTCAAGTCTGCCACACAGGGCAAGAAGGCCCGC
AAGCCCCAGCACCAAAGGGCTGGCTTGTGGTAGCAAGGAAGCTAAGGACCTCAAGGCACGGAGGAAGAAGTCCCAGGATGGCAAGGGCTGCCTGTTGG
ACAGCTCGAGCATGCTGTCGCCTGTGGACTCCCTCGAGTCACCCCATGGCTACTTGTCAGATGTGGCCTCGCCACCCCTCCTCCCCTCCCCATTCCA
GCAGTCTCCATCCATGCCTCTCAGCCACCTGCCTGGTATGCCTGACACTCACCTGGGCATCAGCCACTTGAATGTGGCAGCCAAGCCTGAGATGGCA
GCACTGGCTGGAGGTAGCCGGTTGCCTTTGAGCCACCCCCGOACGCCTCTCCCACCTGCCTGTAGCCTCCAGTGCCAGTCCAGTGCTGAGTACCA
ATGGCACGGGGCTATGAATTTCACCGTGGGTGCACCGGCAAGCTTGAATGGCCAGTGTGAGTGGCTTCCCCGGCTCCAGAATGGCATGGTGCCCAG
CCAGTACAACCCACTACGGCCGGGTGTGACGCCGGGCACACACTGAGCACACAGCAGCTGGCCTCCAGCATAGCATGATGGGGCCACTACACAGCAGC
CTCTCCACCAATACCTTGTCCCCGATTATTTACCAGGGCCTGCCCAACACACGGCTGGCAACACAGCCTCACCTGGTGCAGACCCAGCAGGTGCAGC
CACAGAACTTACAGCTCCAGCCTCAGAACCTGCAGCCACCATCACAGCCACACCTCAGTGTGAGCTCGGCAGCCAATGGGCACCTGGGCCGGAGCTT
CTTGAGTGGGGAGCCCAGTCAGGCAGATGTACAACCGCTGGGCCCCAGCAGTCTGCCTGCACACCATTCTGCCCCAGGAAAGCCAGGCCCTGCCC
ACATCACTGCCATCCTCCATGGTCCCACCCATGACCACTACCCAGTTCCTGACCCCTCCCCTCCGCAGTTACTCCTCCTCCCCTGTGGACAACA
CCCCCAGCCACCAGCTGCAGGTGCCAGAGCACCCCTTCCTCACCCCATCCCCTGAGTCCCCTGACCAGTGGTCCAGCTCCTCCCCGCATTCCAACAT
CTCTGATTGGTCCGAGGGCATCTCCAGCCCGCCCACCACCATGCCGTCCCAGATCACCCACATTCCAGAGGCATTTAAATAAACAGAGATGTGGGAT
GCAGGACCCCAGOTTCCGTTCCCAAGCCCTGTTGGGAGTCCTTTCCAGTGCTTCAGGATGCTGGGGCGACCAAAGGAGCCTTTTAAAAAATGTTTTT
ATACAAAATAAGAGGACAAGAATTTCCAT1TTTTTTTTTTAGTATTTATTTATGTACTTTTATTTTCCACAGAAACACTGCCTTTTTATTTATATGT
ATTGTTTTCTATGGCACTAGGGAAAAACATATCTGTTCCAAGAAAATAAACTAGTTCTCAGAGCCTTGATTTTCCTGGTCAGGGTGAAGTTCCCTGT
GTGTCTGTAAAATATGAACAAGGATTCATGATTTGTAAATGCTGTTTATTTATTGATTGCTTCTTTCCAAAATCGAAAAGAAAGAAAAAAGAACGTG
ACAGGAGAGGGAAGCTGGAAACTGCCATGGCCAGAATTGCCCCTCCCCCACACTCACTGCCCCTCCCCCCAGCGTCACCTGGGATTTGCAGATGTG
TTTAGAAACACGCCCAGACCTTGAACCTTGGGTTCATGGATTAGTTTTGTATCTAAAACAGGAAACAAGTCAGATGATGTGGTTTGTACACTTTCTG
TAACCACCAGTGTGGACTTGAAGAAGTGTCCTCAGCATGTGCAGAGTCTACTACCCAGTACCAGTCGTGAGTCTGCAGGCTCCAGTGTTCTGTAGTA
TAGAGAAAACACTGCCTGGACTGTTCTGTGCATCCCTCCAAACAGCATCATCCAAATCCAACTGAGGACAGACGGACTGTCCCGGCCTGGGCCTGGG
CTCCTAACACCTGACTGCCAAAGGGCTCCAATGTGCATTGTGGACTCGCCAGAGTAGCCTGCATTGAGACTCCAAGAAAACAGAAGCTATGTGGCCT
CTGATCCCCAAACTGGCCTGGGTGGGGACATGCCTTGAGTGTGCTGGAATGTGGGTGGAGCCTGCTTCTGGGCCACCCCTCCTGGTTCAGGGCTGTG
CTCACAGCAGATTCTTGCAGTATCAAGTATACGCCTGTGGCAGAATAAGTATCTGTAAATACATGTTTAAAGATGGATTTTGTTTAAAAAATCTAAA
GGAACAAGTGTGTCGTGTGTCAAGCTGATGAGGACTGECAGACTGTGGCTTAGCTCAGTGTGACCCAGACCTTGTGACCTGTAGCTGCCGAACCAGT
AGCTCCTAAGAGCACAACCCAGGATGGCCCATCTGCTGCCCACCAAGTCCCTTTCCAGCCACTGTGTGCTGGGGGCTTTCGGGGGCAGTTGCCCACC
TCCTCAGGGAGCTCTTTCTGGCCTTTTGGGGGGCAGTGTCTGTGCCATGCCTAATAGATATGACCAGACGCATCCTAAGATGTTGATTCTTACTGT
GTTGTATAAAATAAAGTGTAGTTTACAAAAAAAAGAAACGTAAAAAAAAAAAACTACATGCAAAACTGTAGGTAATGAAAATGATGTATTTTTTTCA
TCTTTTTTTGTTAACTAATTTGCAATAAAAATGATACTGATGGTTGTCCACATTTTGCTAAACTTGGAGTGTGGTTTGGTTCACTTTTTGGGGAGTG
TGACAGAGAAAGGGCCACCCAGCACTGAAATAACCAAATCCAAACCCTCTCTTACACAGAAATACAGAGGTTGCCATAAGGACACACCATTTTCAGA
AGCTGCACAGCGCAGGGCATCAAGGTGGGAAGACAGTTTCCAAAAGAGTTGATGCAGTCCTCTCAGGGATGTGGATCTGATTGATGCCAGCTGTATT
CCTGTGAGCAGCAGAGGAGTGAAGGCACCTTATGTTGTCATAGATGACTATCCTCTGAGCCCAGTAACCCCCAACCTCTTCAAATTCAGCCATGCCT
TGCAAAAGGTCATCCCAGCTGTGCTTGTTGACTGCTTATAGACCCCCTCAAAGAGTTGGGAAGGTCATGGGACTCAAAGGACCCTTGCCTGATGATC
CAGTCACTTTGTTGTATGCAGTCCTGCCCTAATTGTGCATGACCTCAGGGAGGTACTAGAGTATATAGTCTGGGAAGACTAGTGGAGAGACTCCATC
TATGTGGCTGTGAGGAAAGCCTTATCCCTGCTGGGTAAATGTTGTATTAATATTCATTTAATAAAGGCTGGGCGTGGTGGCACACGCCTTTAATCCT
AGCACTCAGGAGGCAGAGGCAGGTGGATTTATGAGTTCGAGGCCAGCCTGGTCTACAGAGTGAGTTCCAGGACAGCCAGGGCTATACAGAGAAACCC
TGTCTCGAAAACCAAAAAAAAAAAAAAAAAAAAAAAAAAAAAAATTCATTTAATAAATCCTGGCAGCAGGTCACTATCCAACTCAATGGTTTATGAA
AGACACACAACCTTTCTTTATATTTTATTATGTCTTATTCAGCACAATAGCTGGGTAGCTGCCTAGCCTCCATATTGCTAAGACTCTGTCTCCCCAT
TCTGAATTCCTGCTTACTAATTCTATGTTCTATCGTGGCTGATCTAGACCAAGTTGGGCAGCCTTATAGGCCATGCTCTCCCAGCTCCTTCAGGTGG
TAGCCTTGCCTCTCTGTCCTGCACTCTTCCCAGGCATGGCATCTCTCCTCTCCCCAACTCTCCCTTCTCCCACTGCCTCAAGTCTCAAGCCCAGGAG
TCCTAAACGGTCCACCTTGTCTGTCCTCCCCAGCTACTGGCTATTGGTATCTTTATTTACCAATCAGAACCCACTGGGGACAGGTTCCCAAAAGCTA
TGTGTAGACCTTCCTGTGCAGTTCGGGGGAACATTAACATTTGTAATACAAACAGCTACAAGTAAAGAGTTCCAATGTGGACTCAGTAAGTAACCCT
TTACCTATGTTCTGCTCTTGTCAGCGCTGCAAATCCATTGGTGCCCTAGTCAGCTTTGGTGCCTAGGACCTTAGGAGGAGGGTTAAGTGTTGTCTCT
TCCTGGGAAGGAATTTTAGCAACACTCATCTAGTCCGAGAGCCCAGCAGGTACAAAGGCATCTTAGCCCTAAGAAGAAATCATAATAGCACAGGTGT
GGATCTCCCTTGGCCTGCTGTACTGGCTAGTTTTGTGTCAACTTGACACAGCTGGAGTTATCACAAAGAAAGGAGTTTCAGTTGGGGAAATGCCTCC
ACGAGATCCAGCTGTAAGGCATTTTCTCTTAGTGATCAAGGGGGGAAGGGCCCTTTGTGGGTGGTACCATCTCTGGGCTGGTAGTCTTGGGTTCTAT
AAGAGAGCAAGCTGAGCAAGCCAGGGGAACCAAGCCAGTAAAGAACATCCCTCCATQGCCTCTGCATCAGTTCCTGCTCCCTGACCTGCTTGAGTTC
CAGTCCTGACTTCCTTGGTGATGAACAGCAGTATGGAAGTGTAAGCCGAATAAACCCTTTCCTCCCCAACTTGTTTCTTGCTCATGATGTTTGTGCA
GGAATAGAAACCCTGACTAAGACACCTGCCTAGGCCTCTCCATTTGGCCCACACCTTGGAGCACAGCAGGAGTCTCTAACCAAAGTGACAATGGGGA
ATGTGATGCCCTCTCTGAGCCTGACATCTGAGGACAGTGTGAATCCATCAAGTTCAGATGTCTTGCCCCGGCTCAGTGTTGCACACTGTGAAAGCG
GAGGGGGCCCAGGGGCCCAGAAGGGGTGCTCACACACAGAATTGTGATAACTTGTCTCATTCTTGCCCTCAAGGGGAAGGTCGGAGCCTGAATTCTG
CCCGGAGAACTAGCATCTCCAAAATTCCATCCGGAATGGAAATGTCAGCTCGGAAAGGGCTTAGCAGGCTCATATGCCTGGGTGTTCTCTAGAGCAT
CATCCACAATGGCATGTGCAGCCCAGGGTTCACAACTGATGGCTGGACCACTGAGAACACATCTGGACTGGGTTCGGGGTGAAAAACATTGATGTTT
GCAGATCAAAGCAAGGGCCTACCCATGTGGGCAAATCTTCTCCAGTGAGAGACCAAACAACAAAAGGTCAGGGAAGAGGGGATGGATCCTGCCTACT
GCGGCAGAACATGAATCCTCTCCCCTCCAGGCTCCCACGGAAAGATTGCCTGTTCTTGGGTCTCCAGCTTGCCAGGTACAGCTCTGGGGACTTGCCA
ACCCCACAACCTCTCTTTGTATCTACCCTAGTAAACATTCAGTGGAGTATTTTTCAGCCATTAGAAGGAACAAAGCTCTGAGACCTACTAACATGTG
GGTGAGCTGCAGGAGGACACAGGGCTCGTGAAAGACATCTCAAAAATGACCACATGATTTCATCTTTATGAAATGTCCTCAGTAAGTAAAGCCACAA
AGACAGAGAACAGAGTGGCCATTCCTGGGTCCAGGGAAATGGGTATGACTGACAATGCCTATACATTTGACTTTGCATGGATAGTCCAGCACTGTGT
ACGTCTAAGGCAGAAGGCTGCATGGCACTGTGAAGGTCCCAGACCCGTCAATGCTACAATTCCACAATCTGTGAGTCTACTAAAACTAGCAAAAAAA
AAAAAAAAAAAAAAATCCAAGCAAAATGGGAACTTTCAGAAATTTGTCCCATGGACACAACAAGTTCTCTTTTATTTTGTGACAGGATCTTGCTGGG
TATCCCAGGTTGGCCTCAATCTCACCATCCTCCTGCCTCAATCTCCCAAGTGTTGGGATTCTAGGTGTGTACCACGCCACCCACTTAAACCTAGTAC
TCTTAAGGAGCATCTATCTGCCCCTAAAAGCCACTGGAAATAAATAAAACATGTAAAACAATATTTTTAAAGGGCTAAAGATGTAGCCTAGTAGTAG
AGGGTTTGTTTACTCTGTGCAAGGCTCTGGATTTCATCCCCAGGACCAATAAAGTAATCAATAAATTGGCTTGAGACAACCTTCAATAAATTCCTT
GTTCTATGAACCTGAGCTCCTCATTTTGAGTCATGTAAAAAAGTTGGGTATGATCATGTGOTGTAATCCCAGCATCTGTGAGATGGGGAGGTAGAGA
TCGGTAGAGTCCTACTTAGCTTTTGTCTAATCAGTGAGTCGCAGGTCCCAGTGAGAGACCTTGTCTCAAAAACTAAAAACAAAAAGGCCGAGGACTG
GGGAGATGGCTCAGATAGTAAAAATTCCTACTGCACTAGCACAAAGACCTGAGTTTGGAATCTCCAGCATCCACATGCTGTCCTGGAATGACCATAC
ATCTGTAATCCTGTGCTGGGGTGGGGAGGGAGACAAGATCCTGGAGCACCACTTGCCTAGCCAATGATCAATCAAGCACTAGGTCCAGTGAGAGAGA
CCCTGCCCCAAAAACCAGGATGGAGTCAGGCATTGTGGCACATGTTTAACCCCAACATTCAGGAGACAGAGGCCCAGGGCTAAAATAGTCTCTGTCT
CAAAAACAAACTAAAAAACCAAGCTGAGAATGAGAATGATACCTTCTGGCTTCCACATGCCATATATACATATACATGCTCACACGCACAGGCACAC
ACATATGCACAGTGTAAGGACTAAATTGGGCACCCAGCAGCTAGACAGCCCAGAGAGAATAGAGTCTTGCAGCCTCAGCCTTCCTTTGTTTAGGG
GGGATTACAGGACAGTGGGCCCTGCTGTCTCTCAACAGAAATACTCCAGGACCAAATCCCAAGCAGACAGAGGTATGGAGATGAGCTCTGTGTCATA
CACACACTGAAGATGGTCATATACAGACATGGGAGCAGAGGCAACTGAGTGGTCTTGGAGCAACTTGCCCTGTGGTACCCGGTCACAGACCTCCAGG
ACAAGGAAATTCCTGTGAAATCCAACTGGTGTGGGGCTGTTTACACAGCAACCTTGGATAGATTGATCCAGTCTCGGGCTGAGGTGATGTCTTGGATG
CCGAAATCTCTCAGGGACACAGGCCAGTTCAGTTCCCTAGGTACTGCCTTTCCCCCACAATGAAATGCCCTCTGTGCTGGCTAGCTGTAACACATCA
ACTTGACACACAAACTACAGAGTTATCTGAGAGGAAGAAACCATAATTGAGAAAATGCCACTGTAAGATCTGGCTGTAAGACATTTTCTTAATTAGT
GATTGATGGAGGAGGGCCAGCTTATGGTCCTGAGTTCTATAAGAAAGTAGGCTGAGCAAGCCATGGGAAACAAGCCAATAAGCAGCACCCCTCCATG
ACCTCTGTCTGTATCAGCTTCTGCCTCTAGGATCCTGCCCTGAGTTCCTGTCCTGACTTTCTTCAGTGATGAACAGTGCTGTCAAAGTGTAAGCCTA
AGAACTCCTTTCCCCTCCAACTTGTTTTTTGGTCGTTGTTTCATCACAGCAATAAAAACCTTAACTAAGACACCACCACTCCCCAGGGATCTGCCAT
CTTGTAATGTTTTTGTTTGTTTTCAATTAGAAGAATAGTTATTATGTATATACTACTAGAGGGCAGTGAGTTAGGCAGTAGTGAGTAAGCAGGGGC
ACACTACGTCCTCTGGCTGGCTGAACCTTGTGATCCTTCTGCCTCAGCCTCCTGAGTTCTAGGATTGCAGGTATGCCCCACCAGGCTTGACTTTAAG
TCCTACTTTTCAACATTCAAGTATGTAAGTAGTTCCAGACAAGTATGGTAAATCCAACACTCTAGAGGCTGAGGCAGGAGGACTGCCATAAGTTCAA
GGCCAGCCTGAGTTATATAGCTAACTTCTGTTTCAAAAATAACAAAAGCAGATATAAATAACCCGAGAACAGCCTGGTGGTGGTGTTGCACACCCTT
AATCCCAGCACTCAGGAGGCAGGCGGAGGTAAATTTCTTGAGTTCCAGGCCAGCCTGGTCTACAGAGTGAGTTTCAGGACAGCCAGGGCTACAAGGA
GAAACCCTGTCTCAAAAAACTGAAACCAAACCAAATAGCAACAAAAACAAACCCAAGAACTAGTTCTCTTCCTGTCCCAAGTGTCCTTCGCTCCACT
GTCTGTGTATATCTTCAGTATGAGAGAGAAGGGCTTCCTTAGCCTCCTGGGGTCATTCAGAGAAGCCCA2TCCAATAGAAATAAGACGGAAACCACA
TAGGTCATTCAAAGTACTTCATAACCCTTAAAAAGGTCAAAGGAGCAGGTGAATTTATTTCAATACTACATCTTGGTTCGTCCAATGAAACCACAGT
TACCACTTAACTAGCAGTCAACACAAGCGAAGTATGAAAAGGACAGAGTTGACATTCTTTGTATTATTATTTGTATTTATTTGCTTATGTATATGTG
TGCTAACACATGCCATACATGTAGAGGTCAGAGGACAAGTGATGAGAGCCAGTCTCTCCCTCTACCATGTGGGTCCTAGGGACTGAACCTGGGTTGT
CCTCCTTGCAGCAGGCACCCTTACCCACTGTGCCAGCTCACCTGCATTATCACCATCTGCACTGGCCATGCTGAAGGTGCTTAGAGGCCACACATG
GTCAAAATGTCATCCCAGACAGGCTAATCTCCACCAGCACAGATAAACAACCCATGGAGCCAGATGTAGTACTGGGAGCACTGGGGGGCGAAGGGGA
GGACGGTAAAGGCAGGAGGTTCGAGTTCTAAGCCAGATCCTGTCTATAAAGATGTAAGCAAAGGAATAAAGAAGGTGAGGGGAGAGACTGCTATG
CTGGTGTTGTTTCTTGTCAAAGGAGGCACCAAATGTGGCTCTGCATTGCTTTGTCCAGCACAAAGAGGGACCCAAGGGTATTAGTGCCATAAACCCA
GCCAAAGCAGCAGGGTTATGCCTACAATCAAAAGTGATGGAGTGCTGCTGTCCTTCTGAGACTCCGGGAACCAGCCAGCCATGAGTCTTGCAAAGTT
TGCTATCAGGGACACTCTATGGGGAGATTTTTACTATGTGACTCCAGACCCCACCCTGTGGAGCTGTGGAGACCCAGGCCAATCCTCAGCTGCATAC
GAGTTAGGAAACAGGGCTCTTTTACTACAGTATCCTCAAGCCCGACTGAGCTATGTGTACTGTGGATTTCGAAAGGTTTGTGTGGAGCTTCTCCAGA
GAATCCCTCTGTTTTGAAGATCAAGGACAGGGATGCTGCTCTCTCCACCTGCACTTTAAAGATGATGTGGTCCTGTCCTAATTCCTGACCACCCTCA
CGGGTGGTGTTCTCATGGACTACCTGGCAGCTGTGGGATAGAAGGTCCCTAGAGTGTCCTCCTGTGTGAGGGGTGACOTCCAAGGTCTATGTGTGGA
CAAGCCCAGCATACACAGTCACTGGTCACCACAAAOTCATTGAGAGATGGTGTCTTAGTTAGGGTTTTACTACTGTGAACAGATATTATGAGCAAGG
CAACTCTTTTTTTTTTAAAGATTTATTTATTATTATATGTAAATACACTGTAGCTGTCTTCAGACATTCCATAGTAGGGCATCAGATCTCATTGTGG
ATGGTTGTGACTACCATGTGGTTGCTGGGATTTGAACTCAGTACCTTTGGAAGAGCAGTCAGTGCTCTTACCCGCTGAGCCATCTCACTAGCCCTG
ATCAAGGCAACTCTTATAAAAACAACATTTAATTGGGGCTGGCTTACAGGTTCTGAGGTTCAGTCCATTATCATCAAGGCAGGAACATGGCAGCATT
CAGGCAGACATGGTACAGAAGGAGCTGAGAGTTCTACAACTTCATCTGAAGGCCACTAGGAGAAGGCCAOTGACTTCCAGGCAGCTAGGACAAGGGT
ATTAAGGCCCACAACCACAGTGACACACCCACTCCAAAAAGGTCACACCTCCTAATAATGCCACTCCCTGGGCTGAGCATATATAAACCATCACAGA
TGGCCAAGAATGGTTGCTTCAATGCTTTTATGTGCACATACCACCTAGAGTGTTCCACAAAGTTCAGGGTTCAGAGCTTTCACACAACTTTGACCTC
AAGGCTTATTTTTAGATTCTAGTGCTTGTGAGATCCATTCTCTACAACACCATGCTATTTCTATGACTATTTCAAATACAAGACTGTGTTATGCCGG
GTGTGGTGGTACATGCCTTTAATCCCAGCACTCAGGAGGCAGAGTCAGCCGGATTTCTGAGTTCGAGGTCAGCCTGGTCTACAAAGTGAGTTCCAGG
ACTTCCAGGGCTACACAGAGAAACCCTGTCTCGAACCCCGAACCCCCCCTCCGCCCCCCAAAAAAGACGACTGTGTTCTGTTTCTCATGAGATTCTT
CTGCATGGGGTGAGCATGCTTTCTACTGGAGCTGTGGACACTCTGGCACTCAGAAACAAGTCTGACCTAAGCTACTTATTTACTCCCCATCAATACA
ATCTTCTTAAGCTGGAAAAGGGAAGGGGAGAGAAAAGGTCTCTGACCTTAGCCTCAGCAGGCCAGGTGGAAGGGGTGACACAGAACAAGGCCCTTTC
CTCTACCTTGAAAGCAATGCCACAGGCTCCCACAAGGTTGAGGATTCTGTCTCTGACCTCAGAGCTTGGAAATCCATTGTCTTCTCTGACTCCAATG
TGCTCAACTCAGAGAGCATTTATGCAGCACCTGCTATCTTTAGTAAGCCCACCCCCTCCAGGGAGTCCAGAAGTAAAGACTTCCCTCTGCGAGGGCT
GCTCTGTGTGTGTGTGTGTGTGTGTGTGTGTGTGTGTGTGGTTAGGGGGGTCTGCAGGGTATGCAACACCTGCACCTAACTCAAGTGAGATAGGATC
TGGAAGGAGCCCTCAATGGATGGATAATTTGTAGGTAGATTTGTAGGTGGCTCTNNNNNNNNNNNNNNNNNNNNNNNNNNNNNNNNNNNNNNNNNNN
NNNNNNNNNNNNNNNNNNNNNNNNNNNNNNNNNWNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNMNNNNNNNNNNNNNNNNNNNNNNNNNNNNNNNNN
NNNNNNNNNNNNNNNNNNNNNNNNNNNNMNNNNNNNNNNNNNNNNMNNNNNNNNNNNNNNNNNNNNNNNMNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
MOUSE SEQUENCE-mRNA
GTGGTGTGCGTCAACGTCCGATCCCCGCCGGCCACCCCAAGAGGCCGCCGCCGGGCTGCGGGCAGCTGGCGAGCAGGCATGCCACOGCTCCTGACGC
CCCTTCTCTGCCTAACGCTGCTGCCCGCGCGCGCCGCAAGAGGCTTGAGATGCTCCCAGCCAAGTGGGACCTGCCTGAATGGAGGTAGGTGCGAAGT
GGCCAGCGGCACTGAAGCCTGTGTTGCCAGCGGCAGCTTTGTGGGCCAACGATGCCAGGACCCCAATCCTTGCCTTCAGCACACGGTGTAAGAATGCT
GGAACGTGCTATGTTGTGGACCATGGTGGCATCGTGGACTATGCCTGCAGCTGTCCCCTGGGTTTCTCTGGGCCCCTCTGCCTGACACCTCTGGACA
AGCCCTGCCTGGCCAACCCCTGCCGCAATGGGGOCACCTGTGACCTGCTCACTCTCACAGAGTACAAGTGCCGCTGCTCTCCAGGGTGGTCAGGAAA
ATCATGTCAGCAGGCTGACCCCTGTGCCTCCAACCCCTGTGCCAATGGTGGCCAGTGCCTGCCCTTTGAGTCTTCATACATCTGTCGCTGCCCGCCT
GGCTTCCATGGCCCCACCTGCAGGCAAGATGTTAATGAGTGCAGCCAGAACCOTGGGCTGTGCCGCCATGGAGGCCACTGCCACAATGAGATCGGCT
CCTATCGCTGTGCCTGCTGTGCCACCCATACTGGTCCCCACTGTGAACTGCCCTATGTGCCCTGCAGCCCCTCACCCTGCCAGAATGGAOCAACCTG
CCGTCCTACAGGGGACACCACCCACGAGTGTGCCTGCTTGCCAGGTTTTGCTGGACAGAACTGTGAAGAAAATGTGGATGACTGTCCAGGAAACAAC
TGCAAGAATGGGGGTGCCTGTGTGGACGGCGTGAATACCTACAATTGCCGCTGCCCACCGGAGGTGACGGGTCAGTACTGTACAGAGGATGTGGACG
AATGTCAGCTCATOCCAAATGCCTGCCAGAATGCGGGAACCTGCCACAACACACACGGCGGCTACAACTGTGTGTGTGTCAATGGGTGGACTGGCGA
GGACTGCAGTGAGAACATTGATGACTGTOCCAGTGCCGCCTGTTTCCAGGGTGCCACGTGCCACGACCGTGTGGCTTCCTTCTACTGCGAATGTCCG
CATGGGCGCACAGGTCTGCTGTGCCACCTCAAGCATGCGTGCATCAGCAACCCCTGCAACGAGGGCTCCAACTGTGACACCAACCCTGTCAACGGCA
AACGAATCTGCACCTGCCCCTCGGGGTACACAGGGCCAGCCTGCAGCCAGGACGTGGATGAGTGTGATCTGGGTGCCAACCGTTGTGAGCACGCAGG
CAAATGCCTCAACACACTGGGTTCTTTTGAGTGCCAGTGTCTACAGGGCTACACGGGACCCGGCTGTGAGATTGATGTTAATGAGTGCATCTCCAAC
CCATGTCAGAATGACGCCACTTGCCTGGACCAGATTGGGGAGTTCCAATGCATATGTATGCCAGGTTATGAAGGTGTATACTGTGAAATCAACACGG
ATGAGTGCGCCAGCAGCCCCTGTCTGCACAATGGCCACTGCATGGACAAGATCCATGAGTTCCAATGTCAGTGCCCCAAAGGCTTCAACGGGCACCT
GTGCCAGTATGATGTGGATGAGTGTGCCAGCACACCATGCAAGAACGGTGCCAAGTGCCTGGATGGGCCCAACACCTATACCTGCGTGTGTACAGAA
GGTTACACAGGGACCCACTGCGAAGTGGACATTGACGAGTGTGACCCTGACCCCTGCCACTATGGTTCCTGTAAGGATGGTGTGGCCACCTTTACCT
GCCTGTGCCAGCCAGGCTACACAGGCCATCACTGTGAGACCAACATCAATGAGTGCCACAGCCAACCGTGCCGCCATGGGGGCACCTGCCAGGACCG
TGACAACTCCTACCTCTGCTTATGCCTCAAGGGAACCAAGGGCCCAACTGTGAGATCAACCTGGATGACTGCGCCAGCAACCCCTGTGACTCTGGC
ACCTGTCTGGACAAGATTGATGGCTACGAATGTGCCTGTGAACCAGGCTACACAGGAAGCATGTGTAACGTCAACATTGACGAATGTGTCGGGCAGCC
CCTGCCACAACGGGGGCACTTGTGAGGATGGCATCGCGGGCTTCACTTGCCGCTGCCCCGAGGGCTACCATGACCCCACGTGCCTGTCCGAGGTCAA
CGAGTGCAACAGTAACCCCTGCATCCACGGAGCTTGCCGGGATGGCCTCAATGGGTACAAGTGTGACTGTGCCCCTGGGTGGAGTGGAACAAACTGT
GACATCAACAACAACGAGTGTGAGTCCAACCCTTGTGTCAACGGTGGCACCTGCAAGGACATGACCAGTGGCTACGTATGCACCTGCCGAGAAGGCT
TCAGTGGCCCTAATTGCCAGACCAACATCAACGAATGTGCCTCCAACCCCTGCCTGAACCAGGGACCTGCATTGATGATGTCGCTGGATACAAGTG
CAACTGTCCTCTGCCATATACAGGAGCCACGTGTGAGGTGGTGTTGGCCCCATGTGCTACCAGCCCCTGCAAAAACAGCGGCGTATGCAAGGAGTCT
GAAGACTATGAGAGTTTCTCCTGTGTCTGTCCCACAGGCTGGCAAGGTCAAACCTGCGAGGTTGACATCAATGAGTGTGTGAAAAGCCCATGTCGCC
ATGGGGCCTCCTGCCAGAACACCATGGCAGCTACCGCTGCCTCTGCCAGGCCGGCTATACAGGTCGCAACTGTGAGAGTGACATCGATGACTGCCG
CCCCAACCCGTGTCACAATGGGGGTTCCTGCACCGATGGCATCAACACAGCCTTCTGCGACTGCCTGCCCGGCTTCCAGGGTGCCTTCTGTGAGGAG
GACATCAATGAATGTGCCAGCAATCCCTGCCAAAATGGAGCCAATTGCACTGACTGTGTGGACAGCTACACATGTACCTGCCCCGTGGGCTTCAATG
GCATCCACTGCGAGAACAACACACCTGACTGTACTGAOAGOTCCTGCTTCAATGGTGGTACCTOTGTGGATGGTATCCTCCTTCACCTGTCTGTG
TCCACCTGGCTTCACGGGCAGCTACTGTCAGTATGATGTCAATGAGTGTGATTCACGGCCCTGTCTGCACGGTGGTACCTGCCAAGACAGCTATGGT
ACTTATAAGTGTACCTGCCCACAGGGCTACACTGGTCTCAACTGCCAGAACCTTGTGCGCTGGTGCGACTCGGCTCCCTGCAAGAATGGTGGCAGGT
GCTGGCAGACCAACACGCAGTACCACTGTGAGTGCCGCAGCGGCTGGACTGGCGTCAACTGCGACGTGCTCAGTGTGTCCTGTGAGGTGGCTGCACA
GAAGCGAGGCATTCACCTCACTCTCCTGTGCCAGCATGGAGGGCTCTGTGTGGATGAGGGAGATAAACATTACTGCCACTGCCAGGCAGGCTACACG
GGCAGCTACTGTGAGGACGAGGTGGACGAGTGCTCACCTAACCCCTGCCAGAATGGAGCTACCTGCACTGACTATCTCGGCGGCTTTTCCTGCAAGT
GTGTGGCTGGCTACCATGGGTCTAACTGCTCCGAGGAGATCCAACGAGTGCCTGTCCCAGCCCTGCCAGAATGGGGGTACCTGCATTGATCTGACCAA
CTCCTACAAGTGTTCCTGCCCCCGGGGGACACAGGGTGTACACTGTGAGATCAATGTTGATGACTGCCATCCCCCCCTTGACCCTGCCTCCCGAAGC
CCCAAGTGCTTCAACAATGGCACCTGTGTGGACCAGGTGGGTGGCTATACCTGCACCTGCCCACCAGGCTTCGTCGGGGAGCGGTGTGAGGGTGATG
TCAATGAATGTCTCTCCAACCCCTGTGACCCACGTGGCACCCAGAACTGTGTGCAGCGTGTTAATGACTTCCACTGCGAGTGCCGGGCTGGCCACAC
TGGACGCCGCTGTGAGTCAGTCATCAATGGCTGCAGGGGCAAACCTTGCAAGAATGGGGGTGTCTGTGCCGTGGCCTCCAACACCGCCCGTGGATTC
ATCTGTAGGTGCCCTGCGGGCTTCGAGGGTGCCACATGTGAGAATGATGCCCGCACTTGTGGCAGCTTACGCTGTCTCAACGGTGGTACATGCATCT
CGGGCCCACGTAGTCCCACCTGCCTATGCCTGGGATCCTTCACCGGCCCTGAGTGCCAGTTCCCAGCCAGCAGCCCCTGTGTGGGTAGCAACCCCTG
CTACAATCAGGGCACCTGTGAGCCCACATCCGAGAACCCTTTCTACCGCTGTCTATGCCCTGCCAAATTCAACGGGCTACTGTGCCACATCCTGGAC
TACAGCTTCACAGGTGGCGCTGGCCCGGACATTCCCCCACCGCAGATTGAGGAGGCCTGTGAGCTGCCTGAGTGCCAGGTGGATGCAGGCAATAAGG
TCTGCAACCTGCAGTGTAATAATCACGCATGTGGCTGGGATGGTGGCGACTGCTCCCTCAACTTCAATGACCCCTGGAAGAACTGCACGCAGTCTCT
ACAGTGCTGGAAGTATTTTAGCGACGGCCACTGTGACAGCCAGTGCAACTCGGCCGGCTGCCTCTTTGATGGCTTCGACTGCCAGCTCACCGAGGGA
CAGTGCAACCCCCTGTATGACCAGTACTGCAAGGACCACTTCAGTGATGGCCACTGCGACCAGGGCTGTAACAGTGCCGAATGTGAGTGGGATGGCC
CTTTCTGCGGGAGCTCAGCCACGTGCTGCACACCAACGTGGTCTTCAAGCGTGATGCGCAAGGCCAGCAGATGATCTTCCCGTACTATGGCCACGAG
GAAGAGCTGCGCAAGCACCCAATCAAGCGCTCTACAGTGGGTTGGGCCACCTCTTCACTGCTTCCTGGTACCAGTGGTGGGCGCCAGCGCAGGGAGC
TGGACCCCATGGACATCCGTGGCTCCATTGTCTACCTGGAGATCGACAACCGGCAATGTGTGCAGTCATCCTCGCAGTGCTTCCAGAGTGCCACCGA
TGTGGCTGCCTTCCTAGGTGCTCTTGCGTCACTTGGCAGCCTCAATATTCCTTACAAGATTGAGGCCGTGAAGAGTGAGCCGGTGGAGCCTCCGCTG
CCCTCGCAGCTGCACCTCATGTACGTGGCAGCGGCCGCCTTCGTGCTCCTGTTCTTTGTGGGCTGTGGGGTGCTGCTGTCCCGCAAGCGCCGGCGCC
AGCATGGCCAGCTCTGGTTCCCTGAGGGTTTCAAAGTGTCAGAGGCCAGCAAGAAGAAGCGGAGAGAGCCCCTCGGCGAGGACTCAGTCGGCCTCAA
GCCCCTGAAGAATGCCTCAGATGGTGCTCTGATGGACGACAATCAGAACGAGTGGGGAGACGAAGACCTGGAGACCAAGAAGTTCCGGTTTGAGGAG
CCAGTAGTTCTCCCTGACCTGAGTGATCAGACTGACCACAGGCAGTGGACCCAGCAGCACCTGGACGCTGCTGACCTGCGCATGTCTGCCATGGCCC
CAACACCGCCTCAGGGGGAGGTGGATGCTGACTGCATGGATGTCAATGTTCGAGGACCAGATGGCTTCACACCCCTCATGATTGCCTCCTGCAGTGG
AGGGGGCCTTGAGACAGGCAACAGTGAAGAAGAAGAAGATGCACCTGCTGTCATCTCTGACTTCATCTACCAGGGCGCCAGCTTGCACAACCAGACA
GACCGCACCGGGGAGACCGCCTTGCACTTGGCTGCCCGATACTCTCGTTCAGATCGTCGAAAGCGCCTTGAGGCCAGTGCAGATGCCAACATCCAGG
ACAACATGGGCCGTACTCCGTTACATGCAGCAGTTTCTGCAGMGCTCAGGGTGTCTTCCAGATCCTGCTCCGGAACAGGGCCACAGATCTGGATGC
CCGAATGCATGATGGCACAACTCCACTGATCCTGGCTGCGCGCCTGGCCGTGGAGGGCATGCTGGAGGACCTCATCAACTCACATGCTGACGTCAAT
GCCGTGGATGACCTAGOCAAGTCGGCTTTGCATTGGGCGGCCGCGGTGAACAATGTGGATGCTGCTGTTGTGCTCCTGAAGAACGGAGCCAACAAGG
ACATCGAGAACAACAAGGAGGAGACTTCCCTGTTCCTGTCGATCCGCCGTGAGAGCTATGAGACTGCCAAAGTGTTGCTGGACCACTTTGCCAACCG
GGACATCACGGATCACATGGACCGATTGCCGCGGGACATCGCACAGGAGCGTATGCACCACGATATCGTGCGGCTTTTGGATGAGTACAACCTGGTG
CGGTCCCCACAGCTGCATGGCACTGCCCTGGGTGGCACACCCACTCTGTCTCCCACACTCTGCTCGCCAAATGGCTACCCTGGCAATCTCAAGTCTG
CCACACAGGGCAAGAAGGCCCGCAAGCCAAGCACCAAAGGGCTGGCTGGTGGTAGCAAGGAAGCTAAGGACCTCAAGGCACGGAGGAAGAGTTCCCA
GGATGGCAAGOGCTGGCTGTTGGACAGCTCGTCGAGCATGCTGTCGCCTGTGGACTCCCTCGAGTCACCCCATGGCTACTTGTCAGATGTGGCCTCG
CACCCCCTCCTCCCCTCCCCATTCCAGCAGTCTCCATCCATGCCTCTCAGCCACCTGCCTGGTATGCCTGACACCCACCTGGGCATCAGCCACTTGA
ATGTGGCAGCCAAGCCTGAGATGGCAGCACTGGCTGGAGGTAGCCGGTTGGCCTTTGAGCACCCCCCGCCACGCCTCTCCCACCTGCCTGTAGCCTC
CAGTOCCTGCCAGTGCTGAGTACCAATGGCACCGGGGCTATGAATTTCACCGTGGGTGCACCGGCAAGCTTGAATGGCCAGTGTGAGTGGCTTCCC
CGGCTCCAGAATGGCATGGTGCCCAGCCAGTACAACCCACTACGGCCGGGTGTGACGCCGGGCACACTGAGCACACAGGCAGCTGGGCTCCAGCATA
GCATGATGGGGCCACTACACAGCAGCCTCTCCACCAATACCTTGTCCCCGATTATTTACCAGGGCCTGCCCAACACACGGCTGGCAACACAGCCTCA
CCTGGTGCAGACCCAGCAGGTGCAGCCACAGAACTTACCACTCCAGCCACAGAACTTACAGCCACCATCACAGCCACACCTCAGTGTGAGCTCGGCA
GCCAATGGGCACCTGGGGCGGAGCTTCTTGAGTGGGGAGCCCAGTCAGGCAGATGTACAACCGCTGGGCCCCAGCAGTCTGCCTGTGCACACCATTC
TGCCCCAOGAAAGCCAGGCCCTACCAACATCACTGCCATCCTCCATGGTCCCACCCATGACCACTACCCAGTTCCTGACCCCTCCATCACAGCACAG
TTACTCCTCCTCCCCTGTGGACAACACCCCCAGCCACCAGCTGCAGGTGCCAGAGCCCACTTTCCTCACCCCATCCCCTGAGTCCCCTGACCAGTGG
TCCAGCTCCTCCCCGCATTCCAACATCTCTGATTGGTCCGAGGGCATCTCCAGCCCGCCCACCACCATGCCGTCCCAGATCACCCACATTCCAGAGG
CATTTAATAACAGAGATGTGGGATGCAGGACCCCAGCTTCCGTTCCCAAGCCCTGTTGGAAGTCCTTTCCAGTGCTTCAGGATGCTGGGGCGACC
AAAGGAGCTTTTTAAAAAATGTTTTTATACAAAATAAGAGGACAAGAATTTCATTTTTTTTTTTAGTATTTATTTATGTACTTTTATTTTCCACAGA
AACACTGCCTTTTTATTTATATGTATTGTTTTCTATGGCACTAGGGGACATATCTGTTCCAAGAAAATAAACTAGTTCTCAGAGCCTTGATTT
TCCTGGTCAGGGTGAAGTTCCCTGTGTGTCTGTAAAATATGAACAAGGATTCATGATTTGTAAATGCTGTTTATTATTGATTGCTTCTTTCCAAAA
TCGAAAAAAAAAA
MOUSE SEQUENCE-CODING
ATGCCACcGCTCCTGACGCCCCTTCTCTGCCTAACGCTGCTGCCCGCGCGCGCCGCAAGAGGCTTGAGATGCTCCCAGCCAAGTGGGACCTGCCTGA
ATGGAGGTAGGTGCGAAGTGGCCAGCGCACTGAAGCCTGTGTTGCCAGCGGCAGCTTTGTGGGCCAACGATGCCAGGACCCCAATCCTTGCCTCAG
CACACGGTGTAAGAATGCTGGAACGTGCTATGTTGTGGACCATGGTGGCATCGTGGACTATGCCTGCAGCTGTCCCCTGGGTTTCTCTGGGCCCCTC
TGCCTGACACCTCTGGACA2GCCCTGCCTGGCCAACCCCTGCCGCAATGGGGGCACCTGTGACCTGCTCACTCTCACAGAGTACAAGTGCCGCTGCT
CTCCAGGGTGGTCAGGAAATCATGTCAGCAGGCTGACCCCGTGCCTCCAACCCCTGTGCCAATGGTGGCCAGTGCCEGCCCTTTGAGTCTTCATA
CATCTGTCGCTGCCCGCCTGGCTTCCATGGCCCCACCTGCAGGCAAGATGTTAATGAGTGCAGCCAGAACCCTGGGCTGTGCCGCCATGGAGGCCAC
TGCCACAATGAGATCGGCTCCTATCGCTGTGCCTGCTGTGCCACCCATACTGGTCCCCACTGTGAACTGCCCTATGTGCCCTGCAGCCCCCTCACCCT
GCCAGAATGGAGCAACCTGCCGTCCTACAGGGGACACCACCCACGAGTGTGCCTGCTTGCCAGGTTTTGCTGGACAGAACTGTGAAGAAAATGTGGA
TGACTGTCCAGGAAACAACTGCAAGAATGGGGGTGCCTGTGTGGACGGCGTGAATACCTACAATTGCCGCTGCCCACCGGAGGTGACGGGTCAGTAC
TGTACAGAGGATGTGGACGAATGTCAGCTCATGCCAAATGCCTGCCAGAATGCGGGAACCTGCCACAACACACACGGCGGCTACAACTGTGTGTGTG
TCAATGGGTGGACTGGCGAGGACTGCAGTGAGAACATTGATGACTGTGCCAGTGCCGCCTGTTTCCAGGGTGCCACTTGCCACGACCGTGTGGCTTC
CTTCTACTGCGAATGTCCGCATGGGCGCACAGGTCTGCTGTGCCACCTCAAGCATGCGTGCATCAGCAACCCCTGCAACGAGGGCTCCAACTGTGAC
ACCAACCCTGTCAACGGCAAACGAATCTGCACCTGCCCCTCGGGGTACACAGGGCCAGCCTGCAGCCAGGACGTGGATGAGTGTGATCTGGGTGCCA
ACCGTTGTGAGCACGCAGGCAAATGCCTCAACACACTGGGTTCTTTTGAGTGCCAGTGTCTACAGGGCTACACGGGACCCGGCTGTGAGATTGATGT
TAATGAGTGCATCTCCAACCCATGTCAGAATGACGCCACTTGCCTGGACCAGATTGGGGAGTTCCAATGCATATGTGTATGCCAGGTTATGAAGGTGTA
TACTGTGAAATCAACACGGATGAGTGCGCCAGCAGCCCCTGTCTGCACAATGGCCACTGCATGGACAAGATCCATGAGTTCCAATGTCAGTGCCCCA
AAGGCTTCAACGGGCACCTGTGCCAGTATGATGTGGATGAGTGTGCCAGCACACCATGCAAGAACGGTGCCAAGTGCCTGGATGGGCCCAACACCTA
TACCTGCGTGTGTACAGAGGTTACACAGGACCCACTGCGAAGTGGACATTGACGAGTGTGACCCTGACCCCTGCCACTATGGTTCCTGTAAGGAT
GGTGTGGCCACCTTTACCTGCCTGTGCCAGCCAGGCTACAGGCCATCACTGTGAGACCAACATCAATGAGTGCCACAGCCAACCGTGCCGCCATG
GGGGCACCTGCCAGGACCGTGACAACTCCTACCTCTGCTTATGCCTCAAGGGAACCACAGGGCCCAACTGTGAGATCAACCTGGATGACTGCGCCAG
CAACCCCTGTGACTCTCGCACCTGTCTGGACAAOATTGATGGCTACGAATGTGCCTGTGAACCAGGCTACACAGGAAGCATGTGTAACGTCAACATT
GACGAATGTGCGGGCAGCCCCTGCCACAACGGGGGCACTTGTGAGGATGGCATCGCGGGCTTCACTTGCCGCTGCCCCGAGGGCTACCATGACCCCA
CGTGCCTGTCCGAGGTCAACGAGTGCAACAGTAACCCCTGCATCCACGGAGCTTGCCGGGATGGCCTCAATGGGTACAAGTGTGACTGTGCCCCTGG
GTGGAGTGGAACAACTGTGACATCACAACAACGAGTGTGAGTCCAACCCTTGTGTCAACGGTGGCACCTGCAACGACATGACCAGTGGCTACGTA
TGCACCTGCCGAGAAGGCTTCAGTGGCCCTAATTGCCAGACCAACATCAACGAATGTGCCTCCAACCCCTGCCTGAACCAGGGGACCTGCATTGATG
ATGTCGCTGGATACAAGTGCAACTGTCCTCTGCCATATACAGGAGCCACGTGTGAGGTSGTGTTGGCCCCATGTGCTACCAGCCCCTGCAAAAACAG
CGGCGTATGCAAGGAGTCTGAAGACTATGAGAGTTTCTCCTGTGTCTGTCCCACAGGCTGGCAAGGTCAAACCTGCGAGGTTGACATCAATGAGTGT
GTGAAAAGCCCATGTCGCCATGGGGCCTCCTGCCAGAACACCAATGGCAGCTACCGCTGCCTCTGCCAGGCCGGCThTACAGGTCGCAACTGTGAGA
GTGACATCGATGACTGCCGCCCCAACCCGTGTCACAATGGGGGTTCCTGCACCGATGGCATCAACACAGCCTTCTGCGACTGCCTGCCCGGCTTCCA
GGGTGCCTTCTGTGAGGAGGACATCAATGAATGTGCCAGCAATCCCTGCCAAAATGGAGCCAATTGCACTGACTGTGTGGACAGCTACACATGTACC
TGCCCCGTGGGCTTCAATGGCATCCACTGCGAGAACAACACACCTGACTGTACTGAGAGCTCCTGCTTCAATGGTGGTACCTGTGTGGATGGTATCA
ACTCCTTCACCTGTCTGTGTCCACCTGGCTTCACGGGCAGCTACTGTCAGTATGATGTCAATGAGTGTGATTCACGGCCCTGTCTGCACGGTGGTAC
CTGCCAAGACAGCTATGGTACTTATAAGTGTACCTGCCCACAGGGCTACACTGGTCTCAACTGCCAGAACCTTGTGCGCTGGTGCGACTCGGCTCCC
TGCAAGAATGGTGGCAGGTGCTGGCAGACCAACACGCAGTACCACTGTGAGTGCCGCAGCGGCTGGACTGGCGTCAACTGCGACGTGCTCAGTGTGT
CCTGTGAGGTGGCTGCACAGAGCGAGGCATTGACGTCACTCTCCTGTGCCAGCATGGAGGGCTCTGTGTGGATGAGGGAGATAAACATTACTGCCA
CTGCCAGGCAGGCTACACGGGCAGCTACTGTGAGGACGAGGTGGACGAGTGCTCACCTAACCCCTGCCAGAATGGAGCTACCTGCACTGACTATCTC
GGCGGCTTTTCCTGCAAGTGTGTGGCTGGCTACCATGGGTCTAACTGCTCCGAGGAGATCAACGAGTGCCTGTCCCAGCCCTGCCAGAATGGGGGTA
CCTGCATTGATCTGACCAACTCCTACAAGTGTTCCTGCCCCCGGGGGACACAGGGTGTACACTGTGAGATCAATGTTGATGACTGCCATCCCCCCCT
TGACCCTGCCTCCCGAAGCCCCAAGTGCTTCAACAATGGCACCTGTGTGGACCAGGTGGGTGGCTATACCTGCACCTGCCCACCAGGCTTCGTCGGG
GAGCGGTGTGAGGGTGATGTCAATGAATGTCTCTCCAACCCCTGTGACCCACGTGGCACCCAGAACTGTGTGCAGCGTGTTAATGACTTCCACTGCG
AGTGCCGGGCTGGCCACACTGGACGCCGCTGTGAGTCAGTCATCAATGGCTGCAGGGGCAAACCTTGCAAGAATGGGGGTGTCTGTGCCGTGGCCTC
CAACACCGCCCGTGGATTCATCTGTAGTGCCCTGCGGGCTTCGAGGGTGCCACATGTGAGAATGATGCCCGCACTTGTGGCAGCTTACGCTGTCTC
AACGGTGGTACATGCATCTCGGGCCCACGTAGTCCCACCTGCCTATGCCTGGGATCCTTCAOCGGCCCTGAGTGCCAGTTCCCAGCCAGCAGCCCCT
GTGTGGGTAGCAACCCCTGCTACAATCAGGGCACCTGTGAGCCCACATCCGAGAACCCTTTCTACCGCTGTCTATGCCCTGCCAAATTCAACGGGCT
ACTGTGCCACATCCTGGACTACAGCTTCACAGGTGGCGCTGGCCCGGACATTCCCCCACCGCAGATTGAGGAGGCCTGTGAGCTGCCTGAGTGCCAG
GTGGATGCAGGCAATAAGGTCTGCAACCTGCAGTGTAATAATCACGCATGTGGCTGGGATGGTGGCGACTGCTCCCTCAACTTCAATGACCCCTGGA
AGAACTGCACGCAGTCTCTACAGTGCTGGAAGTATTTTAGCGACGGCCACTGTGACAGCCAGTGCAACTCGGCCGGCTGCCTCTTTGATGGCTTCGA
CTGCCAGCTCACCGAGGGACAGTGCAACCCCCTGTATGACCAGTACTGCAAGGACCACTTCAGTGATGGCCACTGCGACCAGGGCTGTAACAGTGCC
GAATGTGAGTGGGATGGCCTAGACTGTGCTGAGCATGTACCCGAGCGGCTGGCAGCGGGCACCCTGGTCCTGGTGGTGCTGCTTCCACCCGACCAGC
TACGGAACAACTCCTTCCACTTTCTGCGGGAGCTCAGCCACGTGCTGCACACCAACGTGGTCTTCAAGCGTGATGCGCAAGGCCAGCAGATGATCTT
CCCGTACTATGGCCACGAGGAAGAGCTGCGCAAGCACCCAATCAAGCGCTCTACAGTGGGTTGGGCCACCTCTTCACTGCTTCCTGGTACCAGTGGT
GGGCGCCAGCGCAGGGAGCTGGACCCCATGGACATCCGTGGCTCCATTGTCTACCTGGAGATCGACAACCGGCAATGTGTGCAGTCATCCTCGCAGT
GCTTCCAGAGTGCCACCGATGTGGCTGCCTTCCTAGGTGCTCTTGCGTCACTTGGCAGCCTCAATATTCCTTACAAGATTGAGGCCGTGAAGAGTGA
GCCGGTGGAGCCTCCGCTGCCCTCGCAGCTGCACCTCATGTACGTGGCAGCGGCCGCCTTCGTGCTCCTGTTCTTTGTGGGCTGTGGGGTGCTGCTG
TCCCGCAAGCGCCGGCGCCAGCATGGCCAGCTCTGGTTCCCTGAGGGTTTCAAAGTGTCAGAGGCCAGCAAGAAGAAGCGGAGAGAGCCCCTCGGCG
AGGACTCAGTCGGCCTCAAGCCCCTGAAGAATGCCTCAGATGGTGCTCTGATGGACGACAATCAGAACGAGTGGGGAGACGAAGACCTGGAGACCAA
GAAGTTCCGGTTTGAGGAGCCAGTAGTTCTCCCTGACCTGAGTGATCAGACTGACCACAGGCAGTGGACCCAGCAGCACCTGGACGCTGCTGACCTG
CGCATGTCTGCCATGGCCCCAACACCGCCTCAGGGGGAGGTGGATGCTGACTGCATGGATGTCAATGTTCGAGGACCAGATGGCTTCACACCCCTCA
TGATTGCCTCCTGCAGTGGAGGGGGCCTTGAGACAGGCAACAGTGAAGAAGAAGAAGATGCACCTGCTGTCATCTCTGACTTCATCTACCAGGGCGC
CAGCTTGCACAACCAGACAGACCGCACCGGGGAGACCGCCTTGCACTTGGCTGCCCGATGCTCTCGTTCAGATCGTCGAAAGCGCCTTGAGGCCAGT
GCAGATGCCAACATCCAGGACAACATGGGCCGTACTCCGTTACATGCAGCAGTTTCTGCAGATGCTCAGGGTGTCTTCCAGATCCTGCTCCGGAACA
GGGCCACAGATCTGGATGCCCGAATGCATGATGGCACAACTCCACTGATCCTGGCTGCGCGCCTGGCCGTGGAGGQCATGCTGGAGGACCTCATCAA
CTCACATGCTGACGTCAATGCCGTGGATGACCTAGGCAAGTCGGCTTTGCATTGGGCGGCCGCGGTGAACAATGTGGATGCTGCTGTTGTGCTCCTG
AAGAACGGAGCCAACAAGGACATCGAGAACAACAAGGAGGAGACTTCCCTGT12CCTGTCGATCCGCCGTGAGAGCTATGAGACTGCCAAGTGTTGC
TGGACCACTTTGCCAACCGGGACATCACGGATCACATGGACCGATTGCCGCGGGACATCGCACAGGAGCGTATGCACCACGATATCGTGCGGCTTTT
GGATGAGTACAACCTGGTGCGGTCCCCACAGCTGCATGGCACTGCCCTGGGTGGCACACCCACTCTGTCTCCCACACTCTGCTCGCCAAATGGCTAC
CCTGGCAATCTCAAGTCTGCCACACAGGGCAAGAGGCCCGCAAGCCAAGCACCAAAGGGCTGGCTTGTGGTAGCAAGGAAGCTAAGGACCTCAAGG
CACGGAGGAAGAGTTCCCAGGATGGCAAGGGCTGGCTGTTGGACAGCTCGTCGAGCATGCTGTCGCCTGTGGACTCCCTCGAGTCACCCCATGGCTA
CTTGTCAGATGTGGCCTCGCACCCCCTCCTCCCCTCCCCATTCCAGCAGTCTCCATCCATGCCTCTCAGCCACCTGCCTGGTATGCCTGACACCCAC
CTGGGCATCAGCCACTTGAATGTGGCAGCCAAGCCTGAGATGGCAGCACTGGCTGGAGGTAGCCGGTTGGCCTTTGAGCACCCCCCGCCACGCCTCT
CCCACCTGCCTGTAGCCTCCAGTGCCTGCACAGTGTGAGTACCAATGGCACCGGGGCTATGAATTTCACCGTGGQTGCACCGGCAAGCTTGAATGG
CCAGTGTGAGTGGCTTCCCCGGCTCCAGAATGGCATGGTGCCCAGCCAGTACAACCCACTACGGCCGGGTGTGACGCCGGGCACACTGAGCACACAG
GCAGCTGGGCTCCAGCATAGCATGATGGGGCCACTACACCAGCCTCTCCACCATACCTTGTCCCCGATTATTTACCAGGGCCTGCCCAACACAC
GGCTGGCAACACAGCCTCACCTGGTGCAGACCCAGCAGGTGCAGCCACAGAACTTACCACTCCAGCCACAGAACTTACAGCCACCATCACAGCCACA
CCTCAGTGTGAGCTCGGCAGCCAATGGGCACCTGGGGCGGAGCTTCTTGAGTGGGAGCCCAGTCAGGCAGATGTACAACCGCTGGGCCCCAGCAGT
CTGCCTGTGCACACCATTCTGCCCCAGGAAAGCCAGGCCCTACCCATCACTGCCATCCTCCATGGTCCCACCATGACCACTACCCAGTTCCTGA
CCCCTCCAACAGCACAGTTACTCCTCCTCCCCTGTGGACAAACACCCCCAGCCACCAGCTGCAGGTGCCAGAGAGCCCACTTTCCTCACCCATCCCC
TGAGTCCCCTGACCAGTGGTCCAGCTCCTCCCCGCATTCCAACATCTCTGATTGGTCCGAGGGCATCTCCAGCCCGCCCACCACCATGCCGTCCCAG
ATCACCCACATTCCAGAGGCATTTAAATAA
HUMAN SEQUENCE-GENOMIC
CAGGCCAGGGAGATAATGGGGGACAGGGAAGATAACGCAGTCCCCACGGCTATAAACAGGAAGCTCGGCCATGGGCTTGTGCAGAAAAAGGCCCTCG
GCTGTGCACGTGGTTCCTACATAGCCTTTGCCAGACACCCTGCGTGGCCCGGAGGTCTCCAGGCCGTGCCCCGTGCCCCGACACGCAGGATGTGCT
GGGGCCTGGGCCTGTCCCCTCCATCGCAGCCCTTGTTTGTTGGCCTCCCGTGATGGGCGTTTGCTGCCTGTGACACGGAGCCCCAGGATTAGTTTC
AGGCCTCGGTCTCAGGCCAGGGCCTTTCCTCGTCACCCAGAGATGAGGGGGCTTGCGTCCGGGGTGGGCCCAGGTCCCTGCAGTGTCGTGGAGCCCA
CCCGACTGAGGCCGCCTCTCCTGGTGCAGTCCTTGTTCCTGGGCACGCCTGGTGGGGTGAGGCCTAGGCTGTGCCCCGCTGCCAACCGGGAATGAGG
GGTGGCTTCTGAGCGTGACATTTGTGCAGCTTGTCTTCAGCGTCCCCCATCTGTGTCTCCACTCCTCCCTGGGTCAGGGTCCTGGCACAGCCGGGAG
CGCTCAGGGGTCTCGGTGCACATTTGCCTCCCGGCGGGACCAGCAGACGGCACTCTGATGGCGGAAAGACCAGCAGGCGGTGGCCGATTTGGGAGAT
CCCTCTGGGTGAGGCTCCCGGGGTCACGTGTGTCTCCTTCCCGCAGGTGCCACGTGTGAGGTGGTGCTGGCCCCGTGTGCCCCAGCCCCTGCAGAA
ACGGCGGGGAGTGCAGGCAATCCGAGGACTATGAGAGCTTCTCCTGTGTCTGCCCCACGGGCTGGCAAGGTGAGGCTGGCCAGGGCCCGGTGAGGGC
TGGGATGGGAGGTCAGGATGTCTGCGGGACACAGGCAGCTCCCAGGCAGGCTAGATGAGTCTTTGAAGAGGAGCCGGTGGGTGCTGAGGAGGCCCTG
GTCGGAGAAGTTCTGGAATCAGGAATTGACCTGGGAGCACCGTTCCCAACTCCAGTTCCTGTGACCTTCTTAGGCCAAAATTAGGGGAGAGGGGATG
GTCCTGGGGTCACGGAAGCCCACTCCTGGGTCGGGAGAGGCACTGTAGGTGGGTGGGCCAGCCTGGGAAGGGCCTGGAGGGCCAGGGGCCGCTGGTG
ACCAACCGGCCTCCTCCTGCCCCACAGGGCAGACCTGTGAGGTCGACATCAACGAGTGCGTTCTGAGCCCGTGCCGGCACGGCGCATCCTGCCAGAA
CACCCACGGCGGCTACCGCTGCCACTGCCAGGCCGGCTACAGTGGGCGCAACTGCGAGACCGACATCGACGACTGCCGGCCCAGTGAGTAGCCCCGC
GGCTCTGGCCTCCTCCAGGAAGCTCTCAGGCCTCAGTTCCCCCGGGCAGGGTTGGTGCGCATGGTGCTGGCCATAAGCACCCAGGGAGGCCGGAGTG
TGGCCGGGGAGGGTTGGGTCAGGTGTGGGGGATGTGCTGAGGGATGGCCAGGGTGGTTCAGGAGGGCCCCAGAGCCGGCTTGCTCCTCCCTGCACTT
CGCGGAAGAGTCACGAGAGCCCCTCCCACGGCTCTTCACTGAGCCGAGGATGGCTCGGGCCCGGCCTGCACTCCCGCCTGGCTCATCGGGCCCCGCT
GCAGGCCAGGGCTCTCAGGCCTCCCAGCCCTGCTTCACAGAGGTTGAGGTTGGCAGAAGCCGGGGGTCTTGCTGGCATCACCTGGCAGGCAGAGGCA
AGAACTGTCTCTCCTCCCCTGCTCGGTCTGTGAAGCCTGCAAAGCTGCCCCCTGCCCTGGAGCTTGAGGACAGGAGCAGGTGGGGAGAGAGACCCCA
AGCACAGGAGACGGGTGTGACGCAGCCTGTGGGTGCTGGGGCTCCCCACCAGACACCTTTGTCACAGGGCTGCTTGCCGCAGCCTCGGCAACGAAAG
GCTGGGCTGTCCCCAGCCATGCAGCCTTCCCGGCCCCCTCCOGCAGGTGTGGGTTTGTGCCTGCCCTCACACTCACCCTTCCGTCCTCTCCCAGAC
CCGTGTCACAACGGGGGCTCCTGCACAGACGGCATCAACACGGCCTTCTGCGACTGCCTGCCCGGCTTCCGGGGCACTTTCTGTGAGGAGGACATCA
ACGAGTGTGCCAGTGACCCCTGCCGCAACGGGGCCAACTGCACGGACTGCGTGGACAGCTACACGTGCACCTGCCCCGCAGGCTTCAGCGGGATCCA
CTGTGAGAACAACACGCCTGACTGCACAGAGAGGTGTGCGGGGCTCGAGTGAGGCCGTGGAAGGGAACGGGCGGTGCGGGCCACACGCAGTAGCTGG
CAGCCTGGCACACCATGCAGGCGTCCGCCTAGGCCGGCTGGGCACTTAGCGCTGGCTGCATTGAGTCCAGACATTGTTCATTGTACCGACCTGTCCC
TCCTGAGAGGGCCATTGTGACCTCCTCCTGTGTCCCGCACCAGGAGCGCCGGTAGTCTGTCCTGAAGAACTGGTGCGCTTCCTTCTATTAGACAACG
AGAACTCTGTCCGTTCTTGTTTCCTTTTTGCCGTGTTTGCCAGGAAGCTCATTTGGTAAACGTCAGTCTCGTCCCTGGTTTCTGACGTAATTACCTC
CCGTGTTACCAGGACGGTTTCTGGTTTCTCCCTCATTTACTTTAACCACCCTATGCCCACGCGTTTGGTAAAACCACTCTCGACCTGACTTATAAAG
AAAGCAGGGGAGGGAGGTGTGACGTGGTGTGAGAGCCGGGCCCCGGGTTCCCACTGGCCTCCCTGGGTCAGCCAGGCTGCCCTGGGCCTGTCCTGGC
CATCAGGCCCCTAGGGTTGAGCAGAAGGGGAGGTGCTGGCGAGGCAGAATCTGCTGGAGCGGGGACCCACCAATGCCCTCCGCTCAGCCCCCGCCTG
CCCACCCCCTTGCAGcTCCTGCTTCAACGGTGGCACCTGCGTGGACGGCATCAACTCGTTCACCTGCCTGTGTCCACCCGGCTTCACGGGCAGCTAC
TGCCAGCACGATGTCAATGAGTGCGACTCACAGCCCTGCCTGCATGGCGGCACCTGTCAGGACGGCTGCGGCTCCTACAGGTGCACCTGCCCCCAGG
GCTACACTGGCCCCAACTGCCAGGTGAGTGCGCCGGCCACAGAGGTGCCCGAAGGAGGGGCCCTGGGTGGGTGCCTGCCTGCGGGAGGTGGGAACGG
TCACCCCCAGGTCCCACTGTGTCGGCTCGGGGTCACCCCCACACCCCCGGGCAGAGGGTTTCTGGGGATCTGAGCAGCCGGTGAAGGAACCTGATGC
GGAAGCAGCAGGCACCTTCGTTTCAATCCCAGGTTTCTGGAGCCGGGGCGGGAGCTCAGGAATTGGGGAATTGAGGAAAAGTGTTCCTTCTAGCAAA
GGCCGAGGGTGGTCTGGACCTGCTGAGGGCCCTGAGGGAGACCCAGCCCAGGCTGTTCCTGGTGTGGGGGTGGTGGGGAGTCCAGGGGCGGCAGTCT
CCACTTCTGTAGAATGGGTTGCAGCCTGGGTTGGAGTAGGCCCCTTGGCGGATGTGCGTTCTGAGCTACCGGGGAAATGGCCGGGGCGCGGGCACCC
AGCTGACCCCAATCTGTCCCCAGAACCTTGTGCACTGGTGTGACTCCTCGCCCTGCAAGAACGGCGGCAAATGCTGGCAGACCCACACCCAGTACCG
CTGCGAGTGCCCCAGCGGCTGGACCGGCCTTTACTGCGACGTGCCCAGCGTGTCCTGTGAGGTGGCTGCGCAGCGACAAGGTAACCTGCTGTGCCCA
CCCGGCTCGGGTCCCAGCCCATCAAGGTCCTCTGTGGGCCTGGGCCTCACCTGTCTACCACCCCATCCCCCGCAGGTGTTGACGTTGCCCGCCTGTG
CCAGCATGGAGGGCTCTGTGTGGACGCGGGCAACACGCACCACTGCCGCTGCCAGGCGGGCTACACAGGCAGCTACTGTGAGGACCTGGTGGACGAG
TGCTCACCCAGCCCCTGCCAGAACGGGGCCACCTGCACGGACTACCTGGGCGGCTACTCCTGCAAGGTGGGGGTCCCTCCTAGGGTAAGGGTTGTGG
CCGGCACGAGTGTTGCCACACACCAGGCCCTGGCTGGGAGCTGGCCCAGTGGAGAAAACTGAACCTGATAGGCCCATGCACTGTTCAGTCTCATTAG
GGGAGGGCTGGGGTAATCAGGGTAGACTGCCTGGAAGAGGTGGCCTGTGGAAAGCCTGAAGGAGGGTACCTATACTGAGAAGTGGGATGGGGTTTTC
CCTTCCCCTAGATTGTGTCTGGGCTTGGCCAACACCTACCCTGAGGCCCTCACCTCTATCCTATGGGACGGGGTCCACCCACCCCCAACAGGCAGTG
ACTCCGGTCACCGAGGCCCCGCCAGGGTCTGTTGGGCTGGGTCTCTCTCCAGGTCTGACAGGAGCGAGGGGCCCGTGGCTTCGCTGGACCTGAGGGC
AGCTCATGTGGCCCTGTCAGCCCTCACAGTGGGGTGTGGGAGCACTGCATCCTGGCGCCGGCTGAGCCGAAGGGCCCCTCGTTCTGTCGCCTGCACA
GTGCGTGGCCGGCTACCACGGGGTGAACTGCTCTGAGGAGATCGACGAGTGCCTCTCCCACCCCTGCCAGAACGGGGGCACCTGCCTCGACCTCCCC
AACACCTACAAGTGCTCCTGCCCACGGGGCACTCAGGGTAAGGGCCGCTGCACGGAGGGCTGGTGTTGGCCATCCATGGCCAGGGCAGGGGCAGGGC
AGGCACCCCGGGACCGGCCAGAGGCATCCAGGAACCAGCCAGAAACTCCCGTTTTCCTGTGTGAGGCCAAGGCCGACTCACAGCTGCCCAGGGAAAG
GGCCCAGAGCGGGGGTCCCAGTGGGAAGGGCGTCTCCACGGCACCCTTGACACCTGCCTCTCCCGAGTGTCCGTGCAGCCCCAGACCTGAGCGCTTG
TCTTCCGGGACGGACACGCGGCACGGCAGGGCCGGGGTGTGGCGGGCTTGGGCCACTGACGAAACCTGGCCCCGCAGGTGNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNCCCGGGCCAGGGGTCTCTGGGGGCTTCCTAGGGAGCTCGCTCAGCCTCACTTCTCGAC
CCCTCACCCCCCAGGCCCCTGAAGAACGCTTCAGACGGTGCCCTCATGGACGACAACCAGAATGAGTGGGGGGACGAGGACCTGGAGACCAAGAAGT
TCCGGGTGAGTCGCGAGGCTCCCGGGCTCCTGGGCTCCCGGGCACCTGCTGCCGGGCTGCCCTGACAGGCTCTGCTCACTCCCTCTATGTAGTTCGA
GGAGCCCGTGGTTCTGCCTGACCTGGACGACCAGACAGACCACCGGCAGTGGACTCAGCAGCACCTGGATGCCGCTGACCTGCGCATGTCTGCCATG
GCCCCCACACCGCCCCAGGTGAGGTTGACGCCGACTGCATGGACGTCAATGTCCGCGGGCCTGGTAAGGGTGCCAGCAGCCAGGGCTTCCCTAGCC
CCGTGGCCCACCTGCCTCTCTCCCCTAAGCCCCGAGGCTGGGGTACAGTTGATTCTGGAAACTTAGAATTGGGGTGAGAGCTTTCATCCTTGGGG
GTGTTTCCCATTCAGAGTAGACGTGGGGGGGTCCTGGAGTCCTCCTGTTCTTCCACCCCCCTTCCTGGGGTGATACCGCAGGGCCACTGTGTCCC
TGTAAATTACTCTTTTCTGAAACCTTCTTGAGACATGGAAAGCGTTGATTTTCTTTTTCTTTTTTTTTTTTCTTTTTTTTTTGTTTTTGAGATGGAG
TCTCGCTCCGTCACCCAGGCTGAGTGCAGTGGCACGATCTCGGCTCCCTGCAACCTCCACCTCTCGGGTTCAAGCAATTCTCCTGCCTCAGCCTCC
CCAGTAGCTGGGACTACAGCGCCTGCCACCACACCTGGCTAATTTTTGTATTTTTAGCAGCGATGGGGTTTCACCATGTTGGCCAGGCTGGTCTCG
ACTCCTGACCTCAGGTGATCCGCCCACCTCGGCCTCCCCCAGTGCTAGGATGACAGCGTGAGCCTCCGCATCCTGCTGAAGCATTAATTTTCTAAC
TGCATGCTTCTGGGGTCCTCTTTTTCCTGGGTGGATTTTGGGTGCCAGGTCTTGGGCAGGTCAGGAAGCAGTTGCCCGCCAGGAGTTTAAGCTGGAT
TCGGCTCTGTCCACTGAGCAGCCCAGGCGGACTTCAGCTGCCCCTTGGTGGCTGTGTGCACGGGGCCACCAAGTGCTGGGTGCATGCATCCACACCGT
GGCCCCTTGAGCTTGGGGCTGCTGCCCCCCTCCCCCTGGGCTGCAGCTGGGGACCGGCCCTCCAGACTGAGCACCCGTCTCTGCCTCTGCAGATGGC
TTCACCCCGCTCATGATCGCCTCCTGCAGCGGGGGCGGCCTGGAGACGGGCAACAGCGAGGAAGAGGAGGACGCGCCGGCCGTCATCTCCGACTTCA
TCTACCAGGGCGCCAGCCTGCACAACCAGACAGACCGCACGGGCGAGACCGCCTTGCACCTGGCCGCCCGCTACTCACGCTCTGATGCCGCCAGCG
CCTGCTGGAGGCCAGCGCAGATGCCAACATCCAGGACAACATGGGCCGCACCCCGCTGCATGCGGCTGTGTCTGCCGACGCACAAGGTGTCTTCCAG
GTAGGCAGTGGCTGCCTGTGTGCCCACCTGCCCTCCTCAGGGCCGCCTGGTGGTCTGGGGCAGTGGCCAGGCTTACTGTGGCCCTGGGAGCCTGACCC
CGAGCACAGCTGAGTCCGGGACAACTGGTGCCTCCACCTGGGACCTTCGCAGTCAGCGAGGTGCGAGAGGGGCGTCGGGCCCATCTGTGTTCTC
CAGGGAAGTCAGGCAGAGGCGGGTCTGGCAGGAGGCCTGGGGGATCTGCTGAGTGAGGCAGCACCTCCCACCCCCAGCAAAACAGGCTCCATCAGG
GTTGTGGGCCTTGCTCAAGTCCAGGTTCCACTGCTGCAGCCCCGCAGCCCCGCCCCTCCCTCAACCTTGGCTGCCGGCGTAGCCTGTGGCAGTG
AGAAGCAGGGTTTAGAGGCTGCCGCTCGGTGCCTGCAGACCTAGGGCTCAGCTTGCCGGTGAGCTCGTGGCAAGAATGGATTTAGGGATTTGGATGC
CTGGGTCTCCAGGGAGTGTCCCTGGCAGGGGCTGCCTTTGCAGTCACCCCTGCTGCGAGTCCCCAGGCTCCAGGCGGCCCTGGAGCAAGCAGGTTCA
GATGGGCACAGCCCGGGGAGTTACCACAGAGCTTCTCATTCCTGATTTCTAGCTCAGGTGACACATTGTCATCTTGCAGAAATAAATGTAGGTGA
GCAGAAGAGCGTGGAAGGGAGCCCCGTGCGGGTTTGGTGTGTGCCTCTCTCAGCTGCCTTCTTTGCACAGATGTGGAAGTTCCCAGGTGCTTTGCA
GGAATCAGGCCAAGTTGCCTTTTGCACCCGCCAGTGAGCAGGGGCCATTCCTCCTGCCAGATGCCAAGACCCGGCTGCATTACAAGGGCTGCCCACC
CCCTCTCTGGGCAGAGCCGGACACTAGCTCGGCGGTTCTGAGACGGCTGTAGGGCCGTGAGCCCGGCTTCCTAGTGCCAGCTGTAACCGCCGTTGG
GGGCAGGGACTTGACCTCTCTGTTTTGTAGGTGGTCATGGCAGCTAGGACCACAGTGAGGATTAATGAGGCCACACGTGGTCACGATGGATCCCAC
TGTCACCAAGGGGCCTGTCTGTGGGCAGCACAGCCCCTGCCCCCATCCGGGCCCCTCCTCAGGGGCAGCCCCATGGCGTTTCGTCTTGCCCGGCCGT
GCCGATCTCAGGAGGGTCTCGTCTGTGTCCGGAAGACAGTGGGGCTTCCCGTCCAGGCGTTCGTTCTGGCAGGAGGCATCGGTGTACGTCTGCCCA
GCACCCGCCTGAGCCTCTCCCTGTTGCCCAGATCCTGATCCGGAACCGAGCCACAGACCTGGATGCCCGCATGCATGATGGCACGACGCCACTGATC
CTGGCTGCCCGCCTGGCCGTGGAGGGCATGCTGGAGGACCTCATCAACTCACACGCCGACGTCAACGCCGTAGATGACCTGGGTGAGCCCACGGGGG
CACGGCTGCTCTGTCGTGGGGCGGGACCGCCACAGGGACGGGTGGGACTGGGTTGCCTCCAGCTGGTCTCCAACTACCCCATCTGCTTCTTTCACG
CAGGCAAGTCCGCCCTGCACTGGGCCGCCGCCGTGAACAATGTGGATGCCGCAGTTGTGCTCCTGAAGAACGGGGCTAACAAAGATATGCAGAACAA
CAGGGTGAGCGCGAGGCTGGGATGCCAGGGGAGACGTGAGGGCTGAATCCACACAGAGAAGTGAGGGCCAAACCCACGGGGCGCAGGGACACAAGGGCC
TGACCCACAGGGTCTCGAGGGTCGCTGGGCCCGCATGTGGGCCCCACCCGCATGATCGGGCACCTCGTTAGTGCTCCTGCCCTCCTTCAGCCCCTG
TTTATGGAGCCCTTCCAAGTGCGTCCAGCTGTCAGGACACGGCGGCTCGCCCCTCAGACCCAGCCCTACCCTCCTGGGCGGCAGTCATGGCAGGG
CACACAGCCTGGCGTGGGGAACGCTGCTGCTGCCACTGCTGTGTCACCGTCACCTGGACCTCACCTTCTGTCCCCAGCTGTCACCAGGCGGGGGTGG
GGATGGGATGTGGTGACGCAGGTGGTGCAGATTGAGCCAGAACACGCGTGGCGGCAGCTCCCTGCGGGGCGGGGCCTCTTGGTGTGTTCACCAAGG
CCAGGACCTCAAGGCTCAGAGGAAGAAGTCTCAGGACTATATCCAAGGGGAGCCACCCCCAGCCCTCATCCTGGCCCTGCAGCTCCTGGCCCTGCA
GCTGCTGTTGGTTTCCCCTGGGTGCTCAGGGCACAGGTGCAGACACCCCCACCTCCCTGCCGCCAGAACCCCCACCCCCGCCCCCAACTCCTGCTGC
CCCCTTGGCATGTCAGGCTCAGGCGTCTCTCCCTCCTGGGTGAGGGCACACAGCGGGCCTGGGCACCGGGGCATGTTGGCCCAGGCGCTCCCCAGTC
CGTGGCAGCATGGCCCCACAGAGACTGGGCCCCAGAGGGCATCAAGGCCTGGGAAGCCCCTTCCCACTCCCACACAGCAGCCTCACCCAGACCTGTG
ACGTGTCCACCGCACAGAGGAGACCCCAGGAAGGAGCTTGGTGGCCACCAGGGTGGCTTGATGGCCGTCCGATACGAGCTCCCTCCCTGGTGCCC
TCGCCGGTGCCTCTGAACCGCTCCAGGAATTTCCTTTTGTGCCTTATTGGGGGCAGGGAAGAGGGCCTGCAGTTGGTTAGATTTTCAGTGGGGTCTG
TGACCCCCCCATAGAGGTGGAGCCCCGCTGATCTAGGGTAGAGGACTGCACAGATCCCCTCTCTGGGTGGGTTTCAGAAGATGTATCAAAGCCTTAA
CATTTAACAAGAGTCAGGCTAGGTGGTTGCAGGACGCTGGGGTGGGGTCCTGAGGAGCAGCCTGCCTGCCCCCACCCCGCGGAGGAGGTTGTACTGC
TGCTTCCTCTGGTGATGGAACCTTGGGGAGGGTCCCCACGCCTGGCCTGGCCCCCCTCACCGGCCCCCGCCCTCATCCCCCAGGAGGAGACACCNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNTGCCAACCGGGACATCACGGATCATATGGACCGCCTGCCGCGCGACATC
GCACAGGAGCGCATGCATCACGACATCGTGAGGCTGCTGGACGAGTACAACCTGGTGCGCAGCCCGCAGCTGCACGGAGCCCCGCTGGGGGGCACGC
CCACCCTGTCGCCCCCGCTCTGCTCGCCCAACGGCTACCTGGGCAGCCTCAAGCCCGGCGTGCAGGGCAAGAAGGTCCGCAAGCCCAGCAGCAAAGG
CCTGGCCTGTGGAAGCAAGGAGGCCAAGGACCTCAAGGCACGGAGGAAGAAGTCCCAGGATGGCAAGGGCTGCCTGCTGGACAGCTCCGGCATGCTC
TCGCCCGTGGACTCCCTGGAGTCACCCCATGGCTACCTGTCAGACGTGGCCTCGCCGCCACTGCTGCCCTCCCCGTTCCAGCAGTCTCCGTCCGTGC
CCCTCAACCACCTGCCTGGGATGCCCGACACCCACCTGGGCATCGGGCACCTGAACGTGGCGGCCAAGCCCGAGATGGCGGCGCTGGGTGGGGGCGG
CCGGCTGGCCTTTGAGACTGGCCCACCTCGTCTCTCCCACCTGCCTGTGGCCTCTGGCACCAGCACCGTCCTGGGCTCCAGCAGCGGAGGGGCCCTG
AATTTCACTGTGGGCGGGTCCACCAGTTTGAATGGTCAATGCGAGTGGCTGTCCCGGCTGCAGAGCGGCATGGTGCCGAACCAATACAACCCTCTGC
GGGGGAGTGTGGCACCAGGCCCCCTGAGCACACAGGCCCCCTCCCTGCAGCATGGCATGGTAGGCCCGCTGCACAGTAGCCTTGCTGCCAGCGCCCT
GTCCCAGATGATGAGCTACCAGGGCCTGCCCAGCACCCGGCTGGCCACCCAGCCTCACCTGGTGCAGACCCAGCAGGTGCAGCCACAAAACTTACAG
ATGCAGCAGCAGAACCTGCAGCCAGCAAACATCCAGCAGCAGCAAAGCCTGCAGCCGCCACCACCACCACCACAGCCQCACCTTGGCGTGAGCTCAG
CAGCCAGCGGCCACCTGGGCCGGAGCTTCCTGAGTGGAGAGCCGAGCCAGGCAGACGTGCAGCCACTGGGCCCCAGCAGCCTGGCGGTGCACACTAT
TCTGCCCCAGGAGAGCCCCGCCCTGCCCACGTCGCTGCCATCCTCGCTGGTCCCACCCGTGACCGCAGCCCAGTTCCTGACGCCCCCCTCGCAGCAC
AGCTACTCCTCGCCTGTGGACAACACCCCCAGCCACCAGCTACAGGTGCCTGAGCACCCCTTCCTCACCCCGTCCCCTGAGTCCCCTGACCAGTGGT
CCAGCTCGTCCCCGCATTCCAACGTCTCCGACTGGTCCGAGGGCGTCTCCAGCCCTCCCACCAGCATGCAGTCCCAGATCGCCCGCATTCCGGAGGC
CTTCAAGTAAACGGCGCGCCCCACGAGACCCCGGCTTCCTTTCCCAAGCCTTCGGGCGTCTGTGTGCGCTCTGTGGATGCCAGGGCCGACCAGAGGA
GCCTTTTTAAAACACATGTTTTTATACAAAATAAGAACAAGGATTTTAATTTTTTTTAGTATTTATTTATGTACTTTTATTTTACACAGAAACACTG
CCTTTTTATTTATATGTACTGTTTTATCTGGCCCCAGGTAGAAACTTTTATCTATTCTGAGAAAACAAGCAAGTTCTGAGAGCCAGGGTTTTCCTAC
GTAGGATGAAAAGATTCTTCTGTGTTTATAAAATATAAACAAAGATTCATGATTTATAAATGCCATTTATTTATTGATTCCTTTTTTCAAAATCCAA
AAAGAAATGATGTTGGAGAAGGGAAGTTGAACGAGCATAGTCCAAAAAGCTCCTGGGGCGTCCAGGCCGCGCCCTTTCCCCGACGCCCACCCAACCC
CAAGCCAGCCCGGCCGCTCCACCAGCATCACCTGCCTGTTAGGAGAAGCTGCATCCAGAGGCAAACGGAGGCAAAGCTGGCTCACCTTCCGCACGCG
GATTAATTTGCATCTGAAATAGGAAACAAGTGAAAGCATATGGGTTAGATGTTGCCATGTGTTTTAGATGGTTTCTTGCAAGCATGCTTGTGAAAAT
GTGTTCTCGGAGTGTGTATGCCAAGAGTGCACCCATGGTACCAATCATGAATCTTTGTTTCAGGTTCAGTATTATGTAGTTGTTCGTTGGTTATACA
AGTTCTTGGTCCCTCCAGAACCACCCCGGCCCCCTGCCCGTTCTTGAAATGTAGGCATCATGCATGTCAAACATGAGATGTGTGGACTGTGGCACTT
GCCTGGGTCACACACGGAGGCATCCTACCCTTTTCTGGGGAAAGACACTGCCTGGGCTGACCCCGGTGGCGGCCCCAGCACCTCAGCCTGCACAGTG
TCCCCCAGGTTCCGAAGAAGATGOTCCAGCAACACAGCCTGGGCCCCAGCTCGCGGGACCCGACCCCCCGTGGGCTCCCGTGTTTTCTAGGAGACTT
GCCAGAGCCGGGCACATTGAGCTGTGCAACGCCGTGGGCTGCGTCCTTTGGTCCTGTCCCCGCAGCCCTGGCAGGGGGCATGCGGTCGGGCAGGGGC
TGGAGGGAGGCGGGGGCTGCCCTTGGGCCACCCCTCCTAGTTTGGGAGGAGCAGATTTTTGCAATACCAAGTATAGCCTATGGCAGAAAAAATGTCT
GTAAATATGTTTTTAAAGGTGGATTTTGTTTAAAAAATCTTAATGAATGAGTCTGTTGTGTGTCATGCCAGTGAGGGACGTCAGACTCGGCTCAGCT
CGGGGAGCCTTAGCCGCCCATGCACTGGGGACGCTCCGCTGCCGTGCCGCCTGCACTCCTCAGGGCAGCCTCCCCCGGCTCTACGGGGGCCGCGTGG
TGCCATCCCCAGGGGGCATGACCAGATGCGTCCCAAGATGTTGATTTTTACTGTGTTTTATAAAATAGAGTGTAGTTTACAGAAAAAGACTTTAAAA
GTGATCTACATGAGGAACTGTAGATGATGTATTTTTTTCATCTTTTTTGTTAACTGATTTGCAATAAAAATGATACTGATGGTGATCTGGCTTCCAC
TCCCCTCTGCTCTGGCCTTTGGCTCCCTTTCTGGGAGGGAGGCAGGGCTGCTATGCTCTGAGGGAGCCAGGAGTCGAGGGCCCCTTCTGCTGGGAGA
GTGACGGTGAGGCTGCCTAGTCCTGGCCCACGGGGGTGTGGGGACCACGCTGCCTCCAGGGACTCCATGGTGTCTGCAGCCTGCCTGGTCCAGGCCC
TTTGTAGGGAGATGGACACACAGCAGCAAGGGGGTTGCAGCCCTATGGGAGGTGGGGCTCGTGCCTGGGGTGACACGGCTCCCAGGACACCATGCGT
GAGTCTGGCCCTCCTGGCAGCTCGGGGCTCTTCTCCTCTCAGCCTCGGAGGGATGAAGGTCCCACCCAGCCATCCTGGGGACAGCCCCTCAGGGAGC
TCTGCAGCAGGCAGGGGGCTGCATAGGGAGGGCCTTGAGGCAGTGGCATGACCCCTCTACGGGCTGGAGACCACAGGGAGGACTCTGGCCCCTATAG
GAGGGCAAAGGGAGCTGTCGAAGCCTATGAGCAGGGCAGCATGGGTCGTGGCAGAGTCTGGAAAGTTGTGTGATCCTAAGGAACTGGCTGAGCGAGG
CAGAGGCGGCCTGGTCCTGGGGCCCTGCCCCTGAATAGAGCTGCCAGCCTCACACAAGGGTGGGCCCCTTCTCTCCCCACTGCCTGGGCCTCTGCCC
AGCCCCAGACCTTCAGGGCAGGCCAGTGGCTTCAAACCAGAGCGGTGGGGAGTCTGAGATCCCTCTTTGGATTGCAAAGCACTGCCTGCCCTGGGCC
CAGTCTCTCCAAGGAGGGATGTGAGCCCGAGGCCTCTACTATGGCTGGGGGCTGCGNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNCCGGCATCACTCAGTACAGCCACAGACAGCCTGAGCGTCCACTGCCAACGACGGGTCAGCAAAAACCGTCTGCTGTGAC
GGTGAACCTTAGTGTCAGCTTGGCCGGGCCATTTCGCCAAACGGAGTGTGTCTCTGAGGTGTTCTGGATGAGGTTCGCATTTGCATCCACAGCCTGG
GTAAGGCAGTCCGCCCTCCCCAGTGTGGGTGGGCCCCGTGCGGTCCGCTGAGGCCCGGGGAGAACAATAGGCCGAGTGCCAGGGGTCCTCCCACCCA
ACCGCTTGCACTGGACATTGGTCTTTTCCTGCCTTCAGACTTGGACTGAAAACGTGGGCTCTTCCTGGGCCTGGAGCCCACCGGCCTTCGGACTCGC
AACGCCATCAGGCCTCCTGCCTGCACTGCAGACCCTGGGAACTGCAGGCCTCGAGGTCGTGTGGCCAATTCCCTGCAATAACCTCTCCAGGACTCAC
GTCTTATGGGCTCCGCTTGCCGGAAGAACCCTGACGAATGCGCACGAGGGAATATGATTCCACCGTAGAAAAGGAGGCGCTGACACCCACCATGCGG
ACAAGCCTTGGAAGCACGGTGCTCAGTGAAGGGTGCCGGCCGCAAGGGCCGCAGGTGCAGTGACTCCATTTCTATGGGGTGCCTGTGGTCACCGAA
CCCATGGAGACAGAAAGTAGATTGATGGTTCCCAGAGCCGGGGGGAGGAGGAAAGGGGAGTGGCTGCTAAACGGCCCGGGGTTTTTTTTTGTTTTGT
TTTGTTTTTATACTTTAAGTTTTAGTTTATTGTGGCACTGTTCACAATAGCAAAGACTTGGAACCAACCCAAATGTCCAACAATGATAGACTAGATT
AAGAAATGTGGCACATATATACCATGGAATACTATGCAGCCATAAAAAATGATGAGTTCATGTCCTTTGTAGGGACGTGGATGAAGACGGCCCGGG
GTTTTACTTTGGAGTGACGCAAATGTTCTGGGCCCAGGGAGAGGTGGTGCGTGCACAGCCCTTCGAGTGCACTAAATGCCAATAACGCGGACACTTT
TTTTTTTTTTTTTTTGAGACGGAGTCTCGCTCTGTCACCCAGGCTGGAATCTGGTGGCGTGATCTTGGCTCACTGCAACCTCCGCCTCTGTGTTCAA
GTGATTCTCCTGCCTCAGCCTCCAAAGTAACTGGGATTACAGGCCCCTACCACCACACCCGGCTGATTTTTTTGTATTTTTAGTAGAGATTGGGTTT
CACCATGTTGGCCAGGCTGGTTTCAAACTCCTGATCTCAAGTGATCCACCCGCCTCGGCCTCTTAAAGTGCTGAGGTTACAGGCGTGAGCCACTGCA
CCTGGCCTAACTCGTACACTTTAAAGTGGTTAATTTTAGGTTCTGTGAGTCTCACCTCAATATGTTATTTTTTTTTTTTAATTAAAAAAAGTTTTTG
AGACAGGGTCTTGCTCTGTCACCCAGGCTGGAGTGCAGTGGTGCCATGATAGCTTATTGCAGCCTCAACCTCCTGGGCCCAGGTGATCCTCCCACCT
TGGTCTCTCTAGCAGCTGGGATTACAGGTGCACACCCCTCACCCACCCAATTTTTTTGTTATTTTTTGCAGAGATGGGGTCTCACTTTGTTGCCCA
GGTTGGTCTCGAACTCCTGAGCTCATGCAGTCCTCTCCTCACCTTGGCCTCCCAGAGTGCTGGGATTACAGGTGTGGGCCATTGCGCCTGGCCACAT
ATGCAACTTAAATATTTCAGTAGCCACATTAAAAAAGGTAAAAAGAAACAGCAAGTGTATATTAATACTACAGCTTAGTTAATCCAATATATCAA
AAATATTATTTTAACACAACATAAAAGTGAATGAGTTTTTTTTTGTTGTTTTTTGTTTTTTGAGACAGAGTCTCCCTCTGTCACCAGGCTGGAGT
GCAGTGGTGCGATCTCAGCTCACTGCAACTTCCOCCTCCCGGGTTCAAGCGATTCTCCTGCCTCAGCCTCCCGAGTAGCTGGGATTACAGGCACCTG
CCACCACGCCCAGCTAATTTTTGTATTTTTAGTAGAGAGGGTTTCACCATGTTGGCCAGGCTGGTCTTGATCTCTTGACCTCGTGATCGGCCAC
CTCGGCCTCCCAAGTGCTGGGATTATAGGCGTAAGCCACCACGCCCGGCCGTGAATGAGATGTTTTATATTCTTTTTGTTTGTTTGTTTTTGGTAC
TGTGTCTTTGAAACCCAGGATGTACAGTACACTCACAGCACCCTAGATTGGGCCTGGCCACACTTTAAAAGGTGTTCAGTATCTCCAGGAGGCTCAC
GTCCAGATTCGGACTCTTCAGCAGGATGCCAAGGGGGCTCCAGACAGTTCCACCCAGCGCAGGAAAACACCAAGTGTATCTTCAGCCTGGTGACTCA
CTCCACGCCGCTGCCGGCGTGAACCCCGAGTGTGGCGCTGCGTCCCTCTGGATAGAGCAAAGGAGGGTCCCAAGAGCCCGGAGGCACCAACTCATCA
GGGCCCACAATCAGGCCCCCCGGCAGGCAGGGCTGCCTCCATCCAAGAGGGAAGCACGGTGCCGTAGCCCCCGGGCTGCTCCAGGGATCGACAGGCC
GCCAACGGTGGCCAGTGCCTGCCCTTCGAGGCCTCCTACATCTGCCACTGCCCACCCAGCTTCCATGGCCCCACCTGCCGGCAGGATGTCAACGAGT
GTGGCCAGAAGCCCGGGCTTTGCCGCCACGGAGGCACCTGCCACAACGAGGTCGGCTCCTACCGCTGCGTCTGCCGCGCCACCCGGCACTGGCCCCAA
CTGCGAGCGGCCCTACGTGCCCTGCAGCCCCTCGCCCTGCCAGAACGGGGGCACCTGCCGCCCCACGGGCGACGTCACCCACGAGTGTGCCTGCCTG
CCAGGCTTCACCGGCCAGAACTGTGAGGAAAATATCGACGATTGTCCAGGAAACAACTGCAAGAACGGGGGTGCCTGTGTGGACGGCGTGAACACCT
ACAACTGCCGCTGCCCGCCAGAGTGGACAGGTCAGTACTGTACCGAGGATGTGGACGAGTGCCAGCTGATGCCAAATGCCTGCCAGAACGGCGGGAC
CTGCCACAACACCCACGGTGGCTACAACTGCGTGTGTGTCAACGGCTGGACTGGTGAGGACTGCAGCGAGAACATTGATGACTGTGCCAGCGCCGCC
TGCTTCCACGGCGCCACCTGCCATGACCGTGTGGCCTCCTTCTACTGCGAGTGTCCCCATGGCCGCACAGGTCTGCTGTGCCACCTCAACGACGCAT
GCATCAGCAACCCCTGTAACGAGGGCTCCAACTGCGACACCAACCCTGTCAATGGCAAGGCCATCTGCACCTGCCCCTCGGGGTACACGGGCCCGGC
CTGCAGCCAGGACGTGGATGAGTGCTCGCTGGGTGCCAACCCCTGCGAGCATGCGGGCAAGTGCATCAACACGCTGGGCTCCTTCGAGTGCCAGTGT
CTGCAGGGCTACACGGGCCCCCGATGCGAGATCGACGTCAACGAGTGCGTCTCGAACCCGTGCCAGAACGAOGCCACCTGCCTGGACCAGATTGGGG
AGTTCCAGTGCATCTGCATGCCCGGCTACGAGGGTGTGCACTGCGAGGTCAACACAGACGAGTGTGCCAGCAGCCCCTGCCTGCACAATGGCCGCTG
CCTGGACAAGATCAATGAGTTCCAGTGCGAGTGCCCCACGGGCTTCACTGGGCATCTGTGCCAGTACGATGTGGACGAGTGTGCCAGCACCCCCTGC
AAGAATGGTGCCAAGTGCCTGGACGGACCCAACACTTACACCTGTGTGTGCACGGAAGGGTACACGGGGACGCACTGCGAGGTGGACATCGATGAGT
GCGACCCCGACCCCTGCCACTACGGCTCCTGCAAGGACGGCGTCGCCACCTTCACCTGCCTCTGCCGCCCAGGCTACACGGGCCACCACTGCGAGAC
CAACATCAACGAGTGCTCCAGCCAGCCCTGCCGCCACGGGGGCACCTGCCAGGACCGCGACAACGCCTACCTCTGCTTCTGCCTGAAGGGGACCACA
GGACCCAACTGCGAGATCAACCTGGATGACTGTGCCAGCAGCCCCTGCGACTCGGGCACCTGTCTGGACAAGATCGATGGCTACGAGTGTGCCTGTG
AGCCGGGCTACACAGGGGAGCATGTGTAACATCAACATCGATGAGTGTGCGGGCAACCCCTGCCACAACGGGGGCACCTGCGAGGACGGCATCAATG
GCTTCACCTGCCGCTGCCCCGAGGGCTACCACGACCCCACCTGCCTGTCTGAGGTCAATGAGTGCAACAGCAACCCCTGCGTCCACGGGGCCTGCCG
GGACAGCCTCAACGGGTACAAGTGCGACTGTGACCCTGGGTGGAGTGGGACCAACTGTGACATCAACAACAATGAGTGTGAATCCAACCCTTGTGTC
AACGGCGGCACCTGCAAAGACATGACCAGTGGCTACGTGTGCACCTGCCGGGAGGGCTTCAGCGGTCCCAACTGCCAGACCAACATCAACGAGTGTG
CGTCCAACCCATGTCTGAACCAGGGCACGTGTATTGACGACGTTGCCGGGTACAAGTGCAACTGCCTGCTGCCCTACACAGGTGCCACGTGTGAGGT
GGTGCTGGCCCCGTGTGCCCCCAGCCCCTGCAGAAACGGCGGGGAGTGCAGGCAATCCGAGGACTATGAGAGCTTCTCCTGTGTCTGCCCCACGGGC
TGNNNNNNNNNGGCAGACCTGTGAGGTCGACATCAACGAGTGCGTTCTGAGCCCGTGCCGGCACGGCGCATCCTGCCAGAACACCCACGGCGGCTAC
CGCTGCCACTGCCAGGCCGGCTACAGTGGGCGCAACTGCGAGACCGACATCGACGACTGCCGGCCCAGACCCGTGTCACAACGGGGGCTCCTGCACA
GACGGCATCAACACGGCCTTCTGCGACTGCCTGCCCGGCTTCCGGGGCACTTTCTGTGAGGAGGACATCAACGAGTGTGCCAGTGACCCCTGCCGCA
ACGGGGCCAACTGCACGGACTGCGTGGACAGCTACACGTGCACCTGCCCCGCAGGCTTCAGCGGGATCCACTGTGAGAACAACACGCCTGACTGCAC
AGAGAGCTCCTGCTTCAACGGTGGCACCTGCGTGGACGGCATCAACTCGTTCACCTGCCTGTGTCCACCCGGCTTCACGGGCAGCTACTGCCAGCAC
GATGTCAATGAGTGCGACTCACAGCCCTGCCTGCATGGCGGCACCTGTCAGGACGGCTGCGGCTCCTACAGGTGCACCTGCCCCCAGGGCTACACTG
GCCCCAACTGCCAGAACCTTGTGCACTGGTGTGACTCCTCGCCCTGCAAGAACGGCGGCAAATGCTGGCAGACCCACACCCAGTACCGCTGCCAGTG
CCCCAGCGGCTGGACCGGCCTTTACTGCGACGTGCCCAGCGTGTCCTGTGAGGTGGCTGCGCAGCGACAAGGTGTTGACGTTGCCCGCCTGTGCCAG
CATGGAGGGCTCTGTGTGGACGCGGGCAACACGCACCACTGCCGCTGCCAGGCGGGCTACACAGGCAGCTACTGTGAGGACCTGGTGGACGAGTGCT
CACCCAGCCCCTGCCAGAACGGGGCCACCTGCACGGACTACCTGGGCGGCTACTCCTGCAAGGTGCGTGGCCGGCTACCACGGGGTGAACTGCTCTG
AGGAGATCGACGAGTGCCTCTCCCACCCCTGCCAGAACGGGGGCACCTGCCTCGACCTCCCCAACACCTACAAGTGCTCCTGCCCACGGGGCACTCA
GGGTGTGCACTGTGAGATCAACGTGGACGACTGCAATCCCCCCGTTGACCCCGTGTCCCGGAGCCCCAAGTGCTTTAACAACGGCACCTGCGTGGAC
CAGGTGGGCGGCTACAGCTGCACCTGCCCGCCGGGCTTCGTGGGTGAGCGCTGTGAGGGGGATGTCAACGAGTGCCTGTCCAATCCCTGCGACGCCC
GTGGCACCCAGAACTGCGTGCAGCGCGTCAATGACTTCCACTGCGAGTGCCGTGCTGGTCACACCGGGCGCCGCTGCGAGTCCGTCATCAATGGCTG
CAAAGGCAAGCCCTGCAAGAATGGGGGCACCTGCGCCGTGGCCTCCAACACCGCCCGCGGGTTCATCTGCAAGTGCCCTGCGGGCTTCGAGGGCGCC
ACGTGTGAGAATGACGCTCGTACCTGCGGCAGCCTGCGCTGCCTCAACGGCGGCACATGCATCTCCGGCCCGCGCAGCCCCACCTGCCTGTGCCTGG
GCCCCTTCACGGGCCCCGAATGCCAGTTCCCGGCCAGCAGCCCCTGCCTGGGCGGCAACCCCTGCTACAACCAGGGGACCTGTGAGCCCACATCCGA
GAGCCCCTTCTACCGTTGCCTGTGCCCCGCCAAATTCAACGGGCTCTTGTGCCACATCCTGGACTACAGCTTCGGGGGTGGGGCCGGGCGCGACATC
CCCCCGCCGCTGATCGAGGAGGCGTGCGAGCTGCCCGAGTGCCAGGAGGACGCGGGCAACAAGGTCTGCAGCCTGCAGTGCAACAACCACGCGTGCG
GCTGGGACGGCGGTGACTGCTCCCTCAACTTCAATGACCCCTGGAAGAACTGCACGCAGTCTCTGCAGTGCTGGAAGTACTTCAGTGACGGCCACTG
TGACAGCCAGTGCAACTCAGCCGCCTGCCTCTTCGACGGCTTTGACTGCCAGCGTGCGGAAGGCCAGTGCAACCCCCTGTACGACCAGTACTGCAAG
GACCACTTCAGCGACGGGCACTGCGACCAGGGCTGCAACAGCGCGGAGTGCGAGTGGGACGGGCTGGACTGTGCGGAGCATGTACCCGAGAGGCTGG
CGGCCGGCACGCTGGTGGTGGTGGTGCTGATGCCGCCGGAGCAGCTGCGCAACAGCTCCTTCCACTTCCTGCGGGAGCTCAGCCGCGTGCTGCACAC
CAACGTGGTCTTCAAGCGTGACGCACACGGCCAGCAGATGATCTTCCCCTACTACGGCCGCGAGGAGGAGCTGCGCAAGCACCCCATCAAGCGTGCC
GCCGAGGGCTGGGCCGCACCTGACGCCCTGCTGGGCCAGGTGAAGGCCTCGCTGCTCCCTGGTGGCAGCGAGGGTGGGCGGCGGCGGAGGGAGCTGG
ACCCCATGGACGTCCGCGGCTCCATCGTCTACCTGGAGATTGACAACCGGCAGTGTGTGCAGGCCTCCTCGCAGTGCTTCCAGAGTGCCACCGACGT
GGCCGCATTCCTGGGAGCGCTCGCCTCGCTGGGCAGCCTCIACATCCCCTACAAGATCGAGGCCGTGCAGATGAGACCGTGGAGCCGCCCCCGCCGG
CGCAGCTGCACTTCATGTACGTGGCGGCGGCCGCCTTTGTGCTTCTGTTCTTCGTGGGCTGCGGGGTGCTGCTGTCCCGCAAGCGCCGGCGGCAGCA
TGGCCAGCTCTGGTTCCCTGAGGGCTTCAAAGTGTCTGAGGCCAGCAAGAAGAAGCGGCGGGAGCCCCTCGGCGAGGACTCCGTGGGCCTCAAGCCC
CTGAAGAACGCTTCAGACGGTGCCCTCATGGACAACCAGAATGAGTGGGGGGACGAGGACCTGGAGACCAAGAAGTTCCGGTTCGAGGAGCCCG
TGGTTCTGCCTGACCTGGACGACCAGACAGACCACCGGCAGTGGACTCAGCAGCACCTGGATGCCGCTGACCTGCGCATGTCTGCCATGGCCCCCAC
ACCGCCCCAGGGTGAGGTTGACGCCGACTGCATGGACGTCAATGTCCGCGGGCCTGATGGCTTCACCCCGCTCATGATCGCCTCCTGCAGCGGGGGC
GGCCTGGAGACGGGCAACAGCGAGGAAGAGGAGGACGCGCCGGCCGTCATCTCCGACTTCATCTACCAGGGCGCCAGCCTGCACAACCAGACAGACC
GCACGGGCGAGACCGCCTTGCACCTGGCCGCCCGCTACTCACGCTCTGATGCCGCCAAGCGCCTGCTGGAGGCOAGCGCAGATGCCAACATCCAGGA
CAACATGGGCCGCACCCCGCTGCATGCGGCTGTGTCTGCCGACGCACAAGGTGTCTTCCAGATCCTGATCCGGAACCGAGCCACAGACCTGGATGCC
CGCATGCATGATGGCAOGACGCCACTGATCCTGGCTGCCCGCCTGGCCGTGGAGGGCATGCTGGAGGACCTCATCAACTCACACGCCGACGTCAACG
CCGTAGATGACCTGGGCAAGTCCGCCCTGCACTGGGCCGCCGCCGTGAACAATGTGGATGCCGCAGTTGTGCTCCTGAAGAACGGGGCTAACAAAGA
TATGCAGAACAACAGGGAGGAGACACCCCTGTTTCTGGCCGCCCGGGAGGGCAGCTACGAGACCGCCAAGGTGCTGCTGGACCACTTTGCCAACCGG
GACATCACGGATCTATGGACCGCCTGCCGCGCGACATCGCACAGGAGCGCATGCATCACGACATCGTGAGGCTGCTGGACGAGTACAACCTGGTGC
GCAGCCCGCAGCTGCACGGAGCCCCGCTGGGGGGCACGCCCACCCTGTCGCCCCGCTCTGCTCGCCCAACGGCTACCTGGGCAGCCTCAAGCCCGG
CGTGCAGGGCAAGAAGGTCCGCAAGCCCAGCAGCAAGGCCTGGCCTGTGGAAGCAAGGAGGCCAAGGACCTCAAGGCACGGAGGAAGAAGTCCCAG
GACGGCAAGGGCTGCCTGCTGGACAGCTCCGGCATGCTCTCGCCCGTGGACTCCCTGGAGTCACCCCATGGCTACCTGTCAGACGTGGCCTCGCCGC
CACTGCTGCCCTCCCCGTTCCAGCAGTCTCCGTCCGTGCCCCTCAACCACCTGCCTGGGATGCCCGACACCCACCTGGGCATCGGGCACCTGAACGT
GGCGGCCAAGCCCGAGATGGCGGCGCTGGGTGGGGGCGGCCGGCTGGCCTTTGAGACTGGCCCACCTCGTCTCTCCCACCTCCTGTGGCCTCTGGC
ACCAGCACCGTCCTGGGCTCCAGCAGCGGAGGGGCCCTGAATTTCACTGTGGGCGGGTCCACCAGTTTGAATGGTCAATGCGAGTGGCTGTCCCGGC
TGCAGAGCGGCATGGTGCCGAACCAATACAACCCTCTGCGGGGGAGTGTGGCACCAGGCCCCCTGAGCACACAGGCCCCCCTCCCTGCAGCATGGCAT
GGTAGGCCCGCTGCACGTAGCCTTGCTGCCAGCGCCCTGTCCAGATGATGAGCTACCAGGGCCTGCCCAGCACCCGGCTGGCCACCCAGCCTCAC
CTGGTGCAGACCCAGCAGGTGCAGCCACAACTTACAGATGCAGCAGCAGAACCTGCAGCCAGCCAGCAAACATCCAGCAGCAGCAAAGCCTGCAGCCGC
CACCACCACCACCACAGCCGCACCTTGGCGTGAGCTCAGCAGCCAGCGGCCACCGGCCGGAGCTTCCTGAGTGAGAGCCGAGCCAGGCAGACGT
GCAGCCACTGGGCCCCAGCAGCCTGGCGGTGCACACTAUTCTGCCCCAGGAGAGCCCCGCCCTGCCCACGTCGCTGCCMCCTCGCTGGTCCCACCC
GTGACCGCAGCCCAGTTCCTGACGCCCCCCTCGCAGCACAGCTACTCCTCGCCTGTGGACAACACCCCCAGCCACCAGCTACAGGTGCCTGAGCACC
CCTTCCTCACCCCGTCCCCTGAGTCCCCTGACCAGTGGTCCAGCTCGTCCCCGCATTCCAACGTCTCCGACTGGTCCGAGGGCGTCTCCAGCCCTCC
CACCAGCATGCAGTCCCAGATCGCCCGCATTCCGGAGGCCTTCAAGTAA
HUMAN SEQUENCE-CODING
ATGTACGTGGCGGCGGCCGCCTTTGTGCTTCTGTTCTTCGTGGGCTGCGGGGTGCTGCTGTCCCGCAAGCGCCGGCGGCAGCATGGCCAGCTCTGGT
TCCCTGAGGCTTCAAAGTGTCTGAGGCCAGCAAGAAGAAGCGGCGGGAGCCCCTCGGCGAGGACTCCGTGGGCCTCAAGCCCCIGAAGAACGCTTC
AGACGGTGACGACCAGACAGACCACCGGCAGTGGACTCAGCAGCACCTGGATGCCGCTGACCTGCGCATGTCTGCCATGGCCCCCACACCGCCCCAG
AGGTTGACGCCGACTGCATGGACGTCAATGTCCGCGGGCCTGATGGCTTCACCCCGCTCATGATCGCCTCCTGCAGCGGGGGCGGCCTGGAGACGGG
CAACAGCGAGGAAGAGGAGGACGCGCCGGCCGTCATCTCCGACTTCATCTACCAGGGCGCCAGCCTGCACAACCAGACAGACCGCACGGGCGAGACC
GCCTTGCACCTGGCCGCCCGCTACTCACGCTCTGATGCCGCCAAGCGCCTGCTGGAGGCCAGCGCAGATGCCAACATCCAGGACAACATGGGCCGCA
CCCCGCTGCATGCGGCTGTGTCTGCCGACGCACAAGGTGTCTTCCAGATCCTGATCCGGAACCGAGCCACAGACCTGGATGCCCGCATGCATGATGG
CACGACGCCACTGATCCTGGCTGCCCGCCTGGCCGTGGAGGGCATGCTGGAGGACCTCATCAACTCACACGCCGACGTCAACGCCGTAGATGACCTG
GGCAAGTCCGCCCTGCACTGGGCCGCCGCCGTGAACAATGTGGATGCCGCAGTTGTGCTCCTGAAGAACGGGGCTAACAAAGATATGCAGAACAACA
GGGAGGAGACACCCCTGTTTCTGGCCGCCCGGGAGGGCAGCTACGAGACCGCCAAGGTGCTGCTGGACCACTTTGCCAACCGGGACATCACGGATCA
TATGGACCGCCTGCCGCGCGACATCGCACAGGAGCGCATGCATCACGACATCQTGAGGCTGCTGGACGAGTACAACCTGGTGCGCAGCCCGCAGCTG
CACGGAGCCCCGCTGGGGGGCACGCCCACCCTGTCGCCCCCGCTCTGCTCGCCCAACGGCTACCTGGGCAGCCTCAAGCCCGGCGTGCAGGGCAAGA
AGGTCCGCAAGCCCAGCAGCAAAGGCCTGGCCTGTGGAAGCAAGGAGOCCAAGGACCTCAAGGCACGGAGGAAGAAGTCCCAGGACGGCAAGGGCTG
CCTGCTGGACAGCTCCGGCATGCTCTCGCCCGTGGACTCCCTGGAGTCACCCCATGGOTACCTGTCAGACGTGGCCTCGCCGCCACTGCTGCCCTCC
CCGTTCCAGCAGTCTCCGTCCGTGCCCCTCAACCACCTGCCTGGGATGCCCGACACCCACCTGGGCATCGGGCACCTGAACGTGGCGGCCAAGCCCG
AGATGGCGGCGCTGGGTGGGGGCGGCCGGCTGGCCTTTGAGACTGGCCCACCTCGTCTCTCCCACCTGCCTGTGGCCTCTGGCACCAGCACCGTCCT
GGGCTCCAGCAGCGGAGGGGCCCTGAATTTCACTGTGGGCGGGTCCACCAGTTTGAATGGTCAATGCGAGTGGCTGTCCCGGCTGCAGAGCGGCATG
GTGCCGAACCAATACAACCCTCTGCGGGGGAGTGTGGCACCAGGCCCCCTGAGCACACAGGCCCCCTCCCTGCAGCATGGCATGGTAGGCCCGCTGC
ACAGTAGCCTTGCTGCCAGCGCCCTGTCCCAGATGATGAGCTACCAGGGCCTGCCCAGCACCCGGCTGOCCACCCAGCCTCACCTGGTGCAGACCCA
GCAGGTGCAGCCACAAAACTTACAGATGCAGCAGCAGAACCTGCAGCCAGCAAACATCCAGCAGCAGCAAAGCCTGCAGCCGCCACCACCACCACCA
CAGCCGCACCTTGGCGTGAGCTCAGCAGCCAGCGGCCACCTGGGCCGGAGCTTCCTGAGTGGAGAGCCGAGCCAGGCAGACGTGCAGCCACTGGGCC
CCAGCAGCCTGGCGGTGCACACTATTCTGCCCCAGGAGAGCCCCGCCCTGCCCACGTCGCTGCCATCCTCGCTGGTCCCACCCGTGACCGCAGCCCA
GTTCCTGACGOCCCCCTCGCAGCACAGCTACTCCTCGCCTGTGGACAACACCCCCAGCCACCAGCTACAGGTGCCTGAGCACCCCTTCCTCACCCCG
TCCCCTGAGTCCCCTGACCAGTGGTCCAGCTCGTCCCCGCATTCCAACGTCTCCGACTGGTCCGAGGGCGTCTCCAGCCCTCCCACCAGCATGCAGT
CCCAGATCGCCCGCATTCCGGAGGCCTTCAAGTAA

We claim:

1. A recombinant nucleic acid comprising a nucleotide sequence selected from the group consisting of the sequences outlined in Tables 1-10.

2. A host cell comprising the recombinant nucleic acid of claim 1.

3. An expression vector comprising the recombinant nucleic acid according to claim 2.

4. A host cell comprising the expression vector of claim 3.

5. A recombinant protein comprising an amino acid sequence encoded by a nucleic acid sequence comprising a sequence selected from the group consisting of the sequences outlined in Tables 1-10.

6. A method of screening drug candidates comprising:
a) providing a cell that expresses a carcinoma associated (CA) gene comprising a nucleic acid sequence selected from the group consisting of the sequences outlined in Tables 1-10 or fragment thereof;
b) adding a drug candidate to said cell; and
c) determining the effect of said drug candidate on the expression of said CA gene.

7. A method according to claim 6 wherein said determining comprises comparing the level of expression in the absence of said drug candidate to the level of expression in the presence of said drug candidate.

8. A method of screening for a bioactive agent capable of binding to an CA protein (CAP), wherein said CAP is encoded by a nucleic acid comprising a nucleic acid sequence selected from the group consisting of the sequences outlined in Tables 1-10, said method comprising:
a) combining said CAP and a candidate bioactive agent; and
b) determining the binding of said candidate agent to said CAP.

9. A method for screening for a bioactive agent capable of modulating the activity of an CA protein (CAP), wherein said CAP is encoded by a nucleic acid comprising a nucleic acid sequence selected from the group consisting of the sequences outlined in Tables 1-10, said method comprising:
a) combining said CAP and a candidate bioactive agent; and
b) determining the effect of said candidate agent on the bioactivity of said CAP.

10. A method of evaluating the effect of a candidate carcinoma drug comprising:
a) administering said drug to a patient;
b) removing a cell sample from said patient; and
c) determining alterations in the expression or activation of a gene comprising a nucleic acid sequence selected from the group consisting of the sequences outlined in Tables 1-10.

11. A method of diagnosing carcinoma comprising:
a) determining the expression of one or more genes comprising a nucleic acid sequence selected from the group consisting of the sequences outlined in Tables 1-10, in a first tissue type of a first individual; and
b) comparing said expression of said gene(s) from a second normal tissue type from said first individual or a second unaffected individual; wherein a difference in said expression indicates that the first individual has carcinoma.

12. A method for inhibiting the activity of a CA protein (CAP), wherein said CAP is encoded by a nucleic acid comprising a nucleic acid sequence selected from the group consisting of the sequences outlined in Tables 1-10, said method comprising binding an inhibitor to said CAP.

13. A method of treating carcinomas comprising administering to a patient an inhibitor of an CA protein (CAP), wherein said CAP is encoded by a nucleic acid comprising a nucleic acid sequence selected from the group consisting of the sequences outlined in Tables 1-10.

14. A method of neutralizing the effect of an CA protein (CAP), wherein said CAP is encoded by a nucleic acid comprising a nucleic acid sequence selected from the group consisting of the sequences outlined in Tables 1-10, comprising contacting an agent specific for said CAP protein with said CAP protein in an amount sufficient to effect neutralization.

15. A polypeptide which specifically binds to a protein encoded by a nucleic acid comprising a nucleic acid selected from the group consisting of the sequences outlined in Tables 1-10.

16. A polypeptide according to claim 15 comprising an antibody which specifically binds to a protein encoded by a nucleic acid comprising a nucleic acid sequence selected from the group consisting of the sequences outlined in Tables 1-10.

17. A biochip comprising one or more nucleic acid segments selected from the group consisting of a nucleic acid of the sequences outlined in Tables 1-10 or fragments thereof.

18. A method of diagnosing carcinoma or a propensity to carcinoma by sequencing at least one CA gene of an individual.

19. A method of determining CA gene copy number comprising adding an CA gene probe to a sample of genomic DNA from an individual under conditions suitable for hybridization.
